(19) United States
(12) Patent Application Publication
(10) Pub. No.: US 2026/0132132 A1
(43) Pub. Date: May 14, 2026

(54) 3A,4,5,6-TETRAHYDRO-1 H-PYRAZOLO[3,4-C]PYRIDIN-7(7AH)-ONE DERIVATIVES AS FACTOR XIIA INHIBITORS

(71) Applicant: KALVISTA PHARMACEUTICALS LIMITED, Wiltshire (GB)

(72) Inventors: Rebecca Louise DAVIE, Wiltshire (GB); David Michael EVANS, Wiltshire (GB); Carl Leslie NORTH, Wiltshire (GB); Craig Alex AVERY, Leicestershire (GB); Èric FERRER MALLOFRÉ, Leicestershire (GB); Sean Alan SMITH, Leicestershire (GB)

(21) Appl. No.: 19/121,504

(22) PCT Filed: Oct. 19, 2023
(86) PCT No.: PCT/GB2023/052715

§ 371 Date: Apr. 16, 2025

Related U.S. Application Data
(60) Provisional application No. 63/380,153, filed on Oct. 19, 2022.
(60) Provisional application No. 63/453,385, filed on Mar. 20, 2023.
(60) Provisional application No. 63/510,007, filed on Jun. 23, 2023.

(30) Foreign Application Priority Data
Oct. 19, 2022 (GB) 2215462.9
Mar. 21, 2023 (GB) 2304110.6
Jun. 23, 2023 (GB) 2309514.4

(51) Int. Cl. C07D 471/08; A61K 31/404; A61K 31/416; A61K 31/437; A61K 31/4375; A61K 31/439; A61K 31/4439; A61K 31/444; A61K 31/454; A61K 31/496; A61K 31/497; A61K 31/498; A61K 31/5377; A61K 31/551; C07D 401/12; C07D 401/14; C07D 403/06; C07D 403/12; C07D 405/14; C07D 413/14; C07D 471/04; C07D 519/00

(52) U.S. Cl. CPC C07D 471/08; A61K 31/404; A61K 31/416; A61K 31/437; A61K 31/4375; A61K 31/439; A61K 31/4439; A61K 31/444; A61K 31/454; A61K 31/496; A61K 31/497; A61K 31/498; A61K 31/5377; A61K 31/551; C07D 401/12; C07D 401/14; C07D 403/06; C07D 403/12; C07D 405/14; C07D 413/14; C07D 471/04; C07D 519/00

(57) ABSTRACT

The present invention relates to a pyrazol[3,4 c] pyridine derivative, preparation method and use in medicine thereof. Specifically, the present invention relates to a novel pyrazol[3,4 c] pyridine derivative, preparation method thereof and a pharmaceutical composition comprising the derivative and use thereof as a therapeutic agent, especially their use as Xa factor inhibitors and in the preparation of drugs for treating diseases like thromboembolism.

This invention relates to enzyme inhibitors that are inhibitors of Factor XIIa (FXIIa), and to pharmaceutical compositions comprising, and uses of, such inhibitors.

### BACKGROUND TO THE INVENTION

The compounds of the present invention are inhibitors of Factor XIIa (FXIIa) and thus have a number of possible therapeutic applications, particularly in the treatment of diseases or conditions in which Factor XIIa inhibition is implicated.

FXIIa is a serine protease (EC 3.4.21.38) derived from its zymogen precursor, Factor XII (FXII), which is expressed by the F12 gene. Single chain FXII has a low level of amidolytic activity that is increased upon interaction with negatively charged surfaces and has been implicated in its activation (see Invanov et al., Blood. 2017 Mar. 16;129 (11): 1527-1537. doi: 10.1182/blood-2016-10-744110). Proteolytic cleavage of FXII to heavy and light chains of FXIIa dramatically increases catalytic activity. FXIIa that retains its full heavy chain is αFXIIa. FXIIa that retains a small fragment of its heavy chain is βFXIIa. The separate catalytic activities of αFXIIa and βFXIIa contribute to the activation and biochemical functions of FXIIa. Mutations and polymorphisms in the F12 gene can alter the cleavage of FXII and FXIIa.

FXIIa has a unique and specific structure that is different from many other serine proteases. For instance, the Tyr99 in FXIIa points towards the active site, partially blocking the S2 pocket and giving it a closed characteristic. Other serine proteases containing a Tyr99 residue (e.g. FXa, tPA and FIXa) have a more open S2 pocket. Moreover, in several trypsin-like serine proteases the P4 pocket is lined by an “aromatic box” which is responsible for the P4-driven activity and selectivity of the corresponding inhibitors. However, FXIIa has an incomplete “aromatic box” resulting in more open P4 pocket. See e.g. “Crystal structures of the recombinant β-factor XIIa protease with bound Thr-Arg and Pro-Arg substrate mimetics” M. Pathak et al., Acta. Cryst.2019, D75, 1-14; “Structures of human plasma β-factor XIIa cocrystallized with potent inhibitors” A Dementiev et al., Blood Advances 2018, 2 (5), 549-558; “Design of Small-Molecule Active-Site Inhibitors of the S1A Family Proteases as Procoagulant and Anticoagulant Drugs” P. M. Fischer, J. Med. Chem., 2018, 61 (9), 3799-3822; “Assessment of the protein interaction between coagulation factor XII and corn trypsin inhibitor by molecular docking and biochemical validation” B. K. Hamad et al. Journal of Thrombosis and Haemostasis, 15:1818-1828.

FXIIa converts plasma prekallikrein (PK) to plasma kallikrein (PKa), which provides positive feedback activation of FXII to FXIIa. FXII, PK, and high molecular weight kininogen (HK) together represent the contact system. FXIIa mediated conversion of plasma prekallikrein to plasma kallikrein can cause subsequent cleavage of HK to generate bradykinin, a potent inflammatory hormone that can also increase vascular permeability, which has been implicated in disorders such as hereditary angioedema (HAE). The contact system is activated via a number of mechanisms, including interactions with negatively charged surfaces, negatively charged molecules, unfolded proteins, artificial surfaces, foreign tissue (e.g. biological transplants, that include bio-prosthetic heart valves, and organ/tissue transplants), bacteria, and biological surfaces (including endothelium and extracellular matrix) that mediate assembly of contact system components. In addition, the contact system is activated by plasmin, and cleavage of FXII by other enzymes can facilitate its activation.

Activation of the contact system leads to activation of the kallikrein kinin system (KKS), complement system, and intrinsic coagulation pathway (see https://www.genome.jp/kegg-bin/show_pathway?map04610). In addition, FXIIa has additional substrates both directly, and indirectly via PKa, including Proteinase-activated receptors (PARs), plasminogen, and neuropeptide Y (NPY) which can contribute to the biological activity of FXIIa. Inhibition of FXIIa could provide clinical benefits by treating diseases and conditions associated with these systems, pathways, receptors, and hormones.

PKa activation of PAR₂ mediates neuroinflammation and may contribute to neuroinflammatory disorders including multiple sclerosis (see Göbel et al., Proc Natl Acad Sci USA. 2019 Jan. 2;116 (1): 271-276. doi: 10.1073/pnas.1810020116). PKa activation of PAR₁ and PAR₂ on vascular smooth muscle cells has been implicated in vascular hypertrophy and atherosclerosis (see Abdallah et al., J Biol Chem. 2010 Nov. 5; 285 (45): 35206-15. doi: 10.1074/jbc.M110.171769). FXIIa activation of plasminogen to plasmin contributes to fibrinolysis (see Konings et al., Thromb Res. 2015 August; 136 (2): 474-80. doi: 10.1016/j.thromres.2015.06.028). PKa proteolytically cleaves NPY and thereby alters its binding to NPY receptors (Abid et al., J Biol Chem. 2009 Sep. 11;284 (37): 24715-24. doi: 10.1074/jbc.M109.035253). Inhibition of FXIIa could provide clinical benefits by treating diseases and conditions caused by PAR signaling, NPY metabolism, and plasminogen activation.

FXIIa-mediated activation of the KKS results in the production of bradykinin (BK), which can mediate, for example, angioedema, pain, inflammation, vascular hyperpermeability, and vasodilatation (see Kaplan et al., Adv Immunol. 2014; 121:41-89. doi: 10.1016/B₉₇₈-O-12-800100-4.00002-7; and Hopp et al., J Neuroinflammation. 2017 Feb. 20;14 (1): 39. doi: 10.1186/s12974-017-0815-8). Garadacimab (CSL-312), a monoclonal antibody inhibitory against FXIIa, recently completed a phase 3 study where monthly prophylactic subcutaneous treatment was reported to reduce attacks in patients with type I/II hereditary angioedema (HAE), which results in intermittent swelling of face, hands, throat, gastro-intestinal tract and genitals (see https://www.clinicaltrials.gov/ct2/show/NCT04656418 and Craig et al., The Lancet. 2023;401 (10382): 1079-1090. doi: 10.1016/S0140-6736 (23) 00350-1). Mutations in FXII that facilitate its activation to FXIIa have been identified as a cause of HAE (see Björkqvist et al., J Clin Invest. 2015 Aug. 3;125 (8): 3132-46. doi: 10.1172/JCI77139; and de Maat et al., J Allergy Clin Immunol. 2016 November; 138 (5): 1414-1423.e9. doi: 10.1016/j.jaci.2016.02.021). Since FXIIa mediates the generation of PK to PKa, inhibitors of FXIIa could provide protective effects of all form of BK-mediated angioedema, including HAE and non-hereditary bradykinin-mediated angioedema (BK-AEnH).

“Hereditary angioedema” can be defined as any disorder characterised by recurrent episodes of bradykinin-mediated angioedema (e.g. severe swelling) caused by an inherited genetic dysfunction/fault/mutation. There are currently three known categories of HAE: (i) HAE type 1, (ii) HAE type 2, and (iii) normal C₁ inhibitor HAE (normal C₁-Inh HAE). However, work on characterizing the etiologies of HAE is ongoing so it is expected that further types of HAE might be defined in the future.

Without wishing to be bound by theory, it is thought that HAE type 1 is caused by mutations in the SERPING1 gene that lead to reduced levels of C₁ inhibitor in the blood. Without wishing to be bound by theory, it is thought that HAE type 2 is caused by mutations in the SERPING1 gene that lead to dysfunction of the C₁ inhibitor in the blood. Without wishing to be bound by theory, the cause of normal C₁-Inh HAE is less well defined and the underlying genetic dysfunction/fault/mutation can sometimes remain unknown. What is known is that the cause of normal C₁-Inh HAE is not related to reduced levels or dysfunction of the C₁ inhibitor (in contrast to HAE types 1 and 2). Normal C₁-Inh HAE can be diagnosed by reviewing the family history and noting that angioedema has been inherited from a previous generation (and thus it is hereditary angioedema). Normal C₁-Inh HAE can also be diagnosed by determining that there is a dysfunction/fault/mutation in a gene other than those related to C₁ inhibitor. For example, it has been reported that dysfunction/fault/mutation with plasminogen can cause normal C₁-Inh HAE (see e.g. Veronez et al., Front Med (Lausanne). 2019 Feb. 21; 6:28. doi: 10.3389/fmed.2019.00028; or Recke et al., Clin Transl Allergy. 2019 Feb. 14; 9:9. doi: 10.1186/s13601-019-0247-x.). It has also been reported that dysfunction/fault/mutation with Factor XII can cause normal C₁-Inh HAE (see e.g. Mansi et al. 2014 The Association for the Publication of the Journal of Internal Medicine Journal of Internal Medicine, 2015, 277; 585-593; or Maat et al. J Thromb Haemost. 2019 January; 17 (1): 183-194. doi: 10.1111/jth.14325).

However, angioedemas are not necessarily inherited. Indeed, another class of angioedema is bradykinin mediated angioedema non-hereditary (BK-AEnH), which is not caused by an inherited genetic dysfunction/fault/mutation. Often the underlying cause of BK-AEnH is unknown and/or undefined. However, the signs and symptoms of BK-AEnH are similar to those of HAE, which, without being bound by theory, is thought to be on account of the shared bradykinin mediated pathway between HAE and BK-AEnH. Specifically, BK-AEnH is characterised by recurrent acute attacks where fluids accumulate outside of the blood vessels, blocking the normal flow of blood or lymphatic fluid and causing rapid swelling of tissues such as in the hands, feet, limbs, face, intestinal tract, airway or genitals.

Specific types of BK-AEnH include: non hereditary angioedema with normal C₁ Inhibitor (AE-nC₁ Inh), which can be environmental, hormonal, or drug induced; acquired angioedema; anaphylaxis associated angioedema; angiotensin converting enzyme (ACE) inhibitor induced angioedema; dipeptidyl peptidase 4 inhibitor induced angioedema; and tPA induced angioedema (tissue plasminogen activator induced angioedema). However, reasons why these factors and conditions cause angioedema in only a relatively small proportion of individuals are unknown.

Environmental factors that can induce AE-nC₁ Inh include air pollution (Kedarisetty et al, Otolaryngol Head Neck Surg. 2019 Apr. 30:194599819846446. doi: 10.1177/0194599819846446) and silver nanoparticles such as those used as antibacterial components in healthcare, biomedical and consumer products (Long et al., Nanotoxicology. 2016; 10 (4): 501-11. doi: 10.3109/17435390.2015.1088589).

Various publications suggest a link between the bradykinin and contact system pathways and BK-AEnHs, and also the potential efficacy of treatments, see e.g.: Bas et al. (N Engl J Med 2015; Leibfried and Kovary. J Pharm Pract 2017); van den Elzen et al. (Clinic Rev Allerg Immunol 2018); Han et al (JCI 2002).

For instance, BK-medicated AE can be caused by thrombolytic therapy. For example, tPA induced angioedema is discussed in various publications as being a potentially life threatening complication following thrombolytic therapy in acute stroke victims (see e.g. Simão et al., Blood. 2017 Apr. 20;129 (16): 2280-2290. doi: 10.1182/blood-2016-09-740670; Fröhlich et al., Stroke. 2019 Jun. 11: STROKEAHA₁₁₉₀₂₅₂₆₀. doi: 10.1161/STROKEAHA.119.025260; Rathbun, Oxf Med Case Reports. 2019 Jan. 24;2019 (1): omyl12. doi: 10.1093/omcr/omyl12; Lekoubou et al., Neurol Res. 2014 July; 36 (7): 687-94. doi: 10.1179/1743132813Y.0000000302; Hill et al., Neurology. 2003 May 13;60 (9): 1525-7).

Stone et al. (Immunol Allergy Clin North Am. 2017 August; 37 (3): 483-495.) reports that certain drugs can cause angioedema.

Scott et al. (Curr Diabetes Rev. 2018; 14 (4): 327-333. doi: 10.2174/1573399813666170214113856) reports cases of dipeptidyl Peptidase-4 Inhibitor induced angioedema.

Hermanrud et al., (BMJ Case Rep. 2017 Jan. 10;2017. pii: bcr2016217802) reports recurrent angioedema associated with pharmacological inhibition of dipeptidyl peptidase IV and also discusses acquired angioedema related to angiotensin-converting enzyme inhibitors (ACEI-AAE). Kim et al. (Basic Clin Pharmacol Toxicol. 2019 January; 124 (1): 115-122. doi: 10.1111/bcpt.13097) reports angiotensin II receptor blocker (ARB)-related angioedema. Reichman et al., (Pharmacoepidemiol Drug Saf. 2017 October; 26 (10): 1190-1196. doi: 10.1002/pds.4260) also reports angioedema risk for patients taking ACE inhibitors, ARB inhibitors and beta blockers. Diestro et al. (J Stroke Cerebrovasc Dis. 2019 May; 28 (5): e44-e45. doi: 10.1016/j.jstrokecerebrovasdis.2019.01.030) also reports a possible association between certain angioedemas and ARBs.

Giard et al. (Dermatology. 2012; 225 (1): 62-9. doi: 10.1159/000340029) reports that bradykinin mediated angioedema can be precipitated by estrogen contraception, so called “oestrogen associated angioedema”.

Contact system mediated activation of the KKS has also been implicated in retinal edema and diabetic retinopathy (see Liu et al., Biol Chem. 2013 March; 394 (3): 319-28. doi: 10.1515/hsz-2012-0316). FXIIa concentrations are increased in the vitreous fluid from patients with advance diabetic retinopathy and in Diabetic Macular Edema (DME) (see Gao et al., Nat Med. 2007 February; 13 (2): 181-8. Epub 2007 Jan. 28 and Gao et al., J Proteome Res. 2008 June; 7 (6): 2516-25. doi: 10.1021/pr800112g). FXIIa has been implicated in mediating both vascular endothelial growth factor (VEGF) independent DME (see Kita et al., Diabetes. 2015 October; 64 (10): 3588-99. doi: 10.2337/db15-0317) and VEGF mediated DME (see Clermont et al., Invest Ophthalmol Vis Sci. 2016 May 1;57 (6): 2390-9. doi: 10.1167/iovs.15-18272). FXII deficiency is protective against VEGF induced retinal edema in mice (Clermont et al., ARVO talk 2019). Therefore it has been proposed that FXIIa inhibition will provide therapeutic effects for diabetic retinopathy and retinal edema caused by retinal vascular hyperpermeability, including DME, retinal vein occlusion, age-related macular degeneration (AMD).

As noted above, the contact system can be activated by interaction with bacteria, and therefore FXIIa has been implicated in the treatment of sepsis and bacterial sepsis (see Morrison et al., J Exp Med. 1974 Sep. 1;140 (3): 797-811). Therefore, FXIIa inhibitors could provide therapeutic benefits in treating sepsis, bacterial sepsis and disseminated intravascular coagulation (DIC).

FXIIa mediated activation of the KKS and production of BK have been implicated in neurodegenerative diseases including Alzheimer's disease, multiple sclerosis, epilepsy and migraine (see Zamolodchikov et al., Proc Natl Acad Sci USA. 2015 Mar. 31;112 (13): 4068-73. doi: 10.1073/pnas.1423764112; Simões et al., J Neurochem. 2019 August; 150 (3): 296-311. doi: 10.1111/jnc.14793; Göbel et al., Nat Commun. 2016 May 18; 7:11626. doi: 10.1038/ncomms11626; and https://clinicaltrials.gov/ct2/show/NCT03108469). Therefore, FXIIa inhibitors could provide therapeutic benefits in reducing the progression and clinical symptoms of these neurodegenerative diseases.

FXIIa has also been implicated in anaphylaxis (see Bender et al., Front Immunol. 2017 Sep. 15; 8:1115. doi: 10.3389/fimmu.2017.01115; and Sala-Cunill et al., J Allergy Clin Immunol. 2015 April; 135 (4): 1031-43.e6. doi: 10.1016/j.jaci.2014.07.057). Therefore, FXIIa inhibitors could provide therapeutic benefits in reducing the clinical severity and incidence of anaphylactic reactions.

The role of FXIIa in coagulation was identified over 50 years ago, and has been extensively documented in publications using biochemical, pharmacological, genetic and molecular studies (see Davie et al., Science. 1964 Sep. 18;145 (3638): 1310-2). FXIIa mediated activation of factor XI (FXI) triggers the intrinsic coagulation pathway. In addition, FXIIa can increase coagulation in a FXI independent manner (see Radcliffe et al., Blood. 1977 October; 50 (4): 611-7; and Puy et al., J Thromb Haemost. 2013 July; 11 (7): 1341-52.

doi: 10.1111/jth.12295). Studies on both humans and experimental animal models have demonstrated that FXII deficiency prolongs activated partial prothrombin time (APTT) without adversely affecting hemostasis (see Renne et al., J Exp Med. 2005 Jul. 18;202 (2): 271-81; and Simão et al., Front Med (Lausanne). 2017 Jul. 31; 4:121. doi: 10.3389/fmed.2017.00121). Pharmacological inhibition of FXIIa also prolongs APTT without increasing bleeding (see Worm et al., Ann Transl Med. 2015 October; 3 (17): 247. doi:

10.3978/j.issn.2305-5839.2015.09.07). These data suggest that inhibition of FXIIa could provide therapeutic effects against thrombosis without inhibiting bleeding. Therefore, FXIIa inhibitors could be used to treat a spectrum of prothrombotic conditions including venous thromboembolism (VTE); cancer associated thrombosis; complications caused by mechanical and bioprosthetic heart valves, catheters, extracorporeal membrane oxygenation (ECMO), left ventricular assisted devices (LVAD), dialysis, cardiopulmonary bypass (CPB); sickle cell disease, joint arthroplasty, thrombosis induced by tPA, Paget-Schroetter syndrome and Budd-Chari syndrome. FXIIa inhibitor could be used for the treatment and/or prevention of thrombosis, edema, and inflammation associated with these conditions.

Surfaces of medical devices that come into contact with blood can cause thrombosis. FXIIa inhibitors may also be useful for treating or preventing thromboembolism by lowering the propensity of devices that come into contact with blood to clot blood. Examples of devices that come into contact with blood include vascular grafts, stents, in-dwelling catheters, external catheters, orthopedic prosthesis, cardiac prosthesis, and extracorporeal circulation systems.

Preclinical studies have shown that FXIIa has been shown to contribute to stroke and its complications following both ischemic stroke, and hemorrhagic accidents (see Barbieri et al., J Pharmacol Exp Ther. 2017 March; 360 (3): 466-475. doi: 10.1124/jpet.116.238493; Krupka et al., PLOS One. 2016 Jan. 27;11 (1): e0146783. doi: 10.1371/journal.pone.0146783; Leung et al., Transl Stroke Res. 2012 September; 3 (3): 381-9. doi: 10.1007/s12975-012-0186-5; Simão et al., Blood. 2017 Apr. 20;129 (16): 2280-2290. doi: 10.1182/blood-2016-09-740670; and Liu et al., Nat Med. 2011 February; 17 (2): 206-10. doi: 10.1038/nm.2295). Therefore, FXIIa inhibition may improve clinical neurological outcomes in the treatment of patients with stroke.

FXII deficiency has been shown to reduce the formation of atherosclerotic lesions in Apoe-/-mice (Didiasova et al., Cell Signal. 2018 November; 51:257-265. doi: 10.1016/j.cellsig.2018.08.006). Therefore, FXIIa inhibitors could be used in the treatment of atherosclerosis.

FXIIa, either directly, or indirectly via PKa, has been shown to activate the complement system (Ghebrehiwet et al., Immunol Rev. 2016 November; 274 (1): 281-289. doi: 10.1111/imr.12469). BK increases complement C₃ in the retina, and an in vitreous increase in complement C₃ is associated with DME (Murugesan et al., Exp Eye Res. 2019 Jul. 24; 186:107744. doi: 10.1016/j.exer.2019.107744). Both FXIIa and PKa activate the complement system (see Irmscher et al., J Innate Immun. 2018; 10 (2): 94-105. doi: 10.1159/000484257; and Ghebrehiwet et al., J Exp Med. 1981 Mar. 1;153 (3): 665-76).

A phase 2 study to assess the safety and efficacy of CSL₃₁₂ (garadacimab), a FXIIa inhibitor, in the treatment of COVID-19 has been assigned clinicaltrials.gov identifier NCT04409509. Shatzel et al. (Res Pract Thromb Haemost, 2020 May 15;4 (4): 500-505. doi: 10.1002/rth2.12349) also relates to investigating the contact system's role in COVID-19.

Wygrecka et al. (“Coagulation factor XII regulates inflammatory responses in human lungs”, European Respiratory Journal 2017 50: PA₃₃₉; DOI: 10.1183/1393003.congress-2017.PA₃₃₉) relates to the effect of an accumulation of FXII in acute respiratory distress syndrome (ARDS) lungs.

Wong et al. (“CSL₃₁₂, a Novel Anti-FXII Antibody, Blocks FXII-Induced IL-6 Production from Primary Non-Diseased and Idiopathic Pulmonary Fibrosis Fibroblasts”, American Journal of Respiratory and Critical Care Medicine 2020; 201: A₆₃₆₃) reports that activated FXII may contribute to lung fibrosis (e.g. idiopathic Pulmonary Fibrosis) through direct stimulation of fibroblasts to produce pro-fibrotic cytokine IL-6.

Mckenzie et al. (“A phase I, first-in-human, randomized dose-escalation study of anti-activated factor XII monoclonal antibody garadacimab”, Clin Transl Sci. 2022; 15:626-637. doi: 10.1111/cts.13180) reports on a phase 1 study of garadacimab (CSL₃₁₂), a FXIIa inhibitor, and indicates that garadacimab has the potential to block bradykinin production and thus prevent attacks of edema in HAE. In addition, the reported results are consistent with preclinical studies (Larsson M, Rayzman V, Nolte M W, et al. A factor XIIa inhibitory antibody provides thromboprotection in extracorporeal circulation without increasing bleeding risk. Sci Transl Med. 2014; 6 (222): 222ra17) in which an anti-FXIIa antibody effectively inhibited FXIIa-mediated coagulation and thrombus formation in mouse and rabbit thrombosis models without an increased risk of bleeding events.

Göbel et al. (The Coagulation Factors Fibrinogen, Thrombin, and Factor XII in Inflammatory Disorders—A Systematic Review, Front. Immunol., 26 Jul. 2018 | https://doi.org/10.3389/fimmu.2018.01731) relates to FXIl's role in the rheumatoid arthritis (RA).

Scheffel et al. (Cold-induced urticarial autoinflammatory syndrome related to factor XII activation, Nature Communications volume 11, Article number: 179 (2020)) reports that there is a link between contact system activation and cytokine-mediated inflammation, such as cold-induced urticarial autoinflammatory syndrome.

Peyrou et al. (The kallikrein-kinin pathway as a mechanism for auto-control of brown adipose tissue activity, NATURE COMMUNICATIONS, (2020) 11:2132, https://doi.org/10.1038/s41467-020-16009-x), reports a pathway for controlling brown adipose tissue (BAT) thermogenic activity mediated by the kallikrein-kinin system, which may contribute to expanding the range of potential pharmacological candidates in therapeutic strategies against obesity and associated diseases designed to improve energy expenditure and remove excess blood metabolites through activation of BAT. Impaired BAT activity is associated with obesity and insulin resistance.

Wygrecka et al. (“Use of a FXIIa-Inhibitor in the treatment of renal fibrosis and/or kidney disease” WO2019/113642) relates to an inhibitor of Factor XII for use in treating or preventing kidney disease, renal fibrosis, glomerulosclerosis, renal scarring, ischemia/reperfusion injury in native or transplant kidneys and/or acute kidney injury.

Nicoletti et al. (“Primary Role for Kinin B₁ and B₂ Receptors in Glioma Proliferation”, Mol Neurobiol (2017) 54:7869-7882; DOI 10.1007/s12035-016-0265-9) reports that brain glioblastoma growth and malignancy, as well as complications thereof including oedema and cognitive function, may be beneficially affected by modulation of B₁ and B₂ kinin receptors, part of the kallikrein-kinin system, which are activated by bradykinin.

Gamboa et al. (“Bradykinin B₂ receptor blockade and intradialytic hypotension”, BMC Nephrology (2023) 24:134, https://doi.org/10. 2:023:03192:4) reports that blocking the effect of bradykinin or inhibiting the activation of the kallikrein-kinin system has a therapeutic effect in treating and preventing the occurrence of intradialytic hypotension (IDH).

Compounds that are said to be FXIIa inhibitors have been described by Rao et al. (“Factor XIIa Inhibitors” WO2018/093695), Hicks et al. (“Factor XIIa Inhibitors” WO2018/093716), Breslow et al. (“Aminotriazole immunomodulators for treating autoimmune diseases” WO2017/123518) and Ponda et al. (“Aminacylindazole immunomodulators for treatment of autoimmune diseases” WO2017/205296 and “Pyranopyrazole and pyrazolopyridine immunomodulators for treatment of autoimmune diseases” WO2019/108565). FXII/FXIIa inhibitors are said to have been described by Nolte et al. (“Factor XII inhibitors for the administration with medical procedures comprising contact with artificial surfaces” WO2012/120128).

Compounds that are said to be modulators of FXIIa have been described by Philippou et al. (“Factor XIIa Inhibitors” WO 2019/211585 and WO 2019/186164). Macrocyclic peptides that are said to be inhibitors of FXIIa have been described by Wilbs et al. (Nat Commun 11, 3890 (2020). Doi: 10.1038/s41467-020-17648-w).

To date, no FXIIa inhibitors have been approved for medical use, and there are no small molecule FXIIa inhibitors in clinical development. Although certain known compounds are said to be modulators or inhibitors of FXIIa, these compounds can suffer from limitations such as being non-reversible or covalent binders, being poorly selective for FXIIa over other related enzymes, or not having demonstrated pharmacokinetic properties suitable for oral therapy. For example, compounds with acylating reactivity e.g. acylated aminotriazoles, are typically non-reversible covalent binders, and can sometimes also be unstable in water and/or blood plasma due to their inherent reactivity. Poor selectivity for FXIIa over other serine proteases (such as thrombin, FXa, FXIa, KLK1, plasmin, trypsin) increases the risk of off-target effects. An example of an undesirable off-target effect is bleeding. Off-target effects can be made even worse (i.e. there is typically a higher likelihood of poor selectivity and off-target effects) if the inhibitor is a covalent binder. Therefore, there remains a need to develop new FXIIa inhibitors that are highly selective for FXIIa in order to e.g. mitigate the risks of non-selectivity and cytotoxicity. There also remains a need to develop new FXIIa inhibitors that are not covalent inhibitors to mitigate the risks of non-selectivity and cytotoxicity. There is a particular need to develop small molecule FXIIa inhibitors, particularly as an oral therapy.

In view of the above, there also remains a need to develop new FXIIa inhibitors that will have utility to treat a wide range of disorders, in particular angioedema; HAE, including: (i) HAE type 1, (ii) HAE type 2, and (iii) normal C₁ inhibitor HAE (normal C₁-Inh HAE); BK-AEnH, including AE-nC₁ Inh, ACE and tPA induced angioedema; vascular hyperpermeability; stroke including ischemic stroke and haemorrhagic accidents; retinal edema; diabetic retinopathy; DME; retinal vein occlusion; AMD; neuroinflammation; neuroinflammatory/neurodegenerative disorders such as MS (multiple sclerosis); other neurodegenerative diseases such as Alzheimer's disease, epilepsy and migraine; sepsis; bacterial sepsis; inflammation; anaphylaxis; thrombosis; thromboembolism caused by increased propensity of medical devices that come into contact with blood to clot blood; prothrombotic conditions including disseminated intravascular coagulation (DIC), venous thromboembolism (VTE), cancer associated thrombosis, complications caused by mechanical and bioprosthetic heart valves, complications caused by catheters, complications caused by ECMO, complications caused by LVAD, complications caused by dialysis, complications caused by CPB, sickle cell disease, joint arthroplasty, thrombosis induced to tPA, Paget-Schroetter syndrome and Budd-Chari syndrome; atherosclerosis; COVID-19; COVID-19 associated with SARS-COV-2 infection; acute respiratory distress syndrome (ARDS); idiopathic pulmonary fibrosis (IPF); rheumatoid arthritis (RA); cold-induced urticarial autoinflammatory syndrome; obesity; diabetes; kidney disease; renal fibrosis; glomerulosclerosis; renal scarring; ischemia/reperfusion injury in native or transplant kidneys; acute kidney injury; brain glioblastoma growth and malignancy, and complications thereof including oedema and cognitive function; and intradialytic hypotension (IDH). In particular, there remains a need to develop new FXIIa inhibitors.

### DESCRIPTION OF THE INVENTION

The present invention relates to a series of inhibitors of Factor XIIa (FXIIa). The compounds of the invention are potentially useful in the treatment of diseases or conditions in which Factor XIIa inhibition is implicated. The invention further relates to pharmaceutical compositions of the inhibitors, to the use of the compositions as therapeutic agents, and to methods of treatment using these compositions.

Specifically, the invention provides compounds of Formula (I),

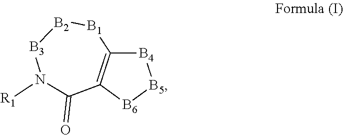

wherein:
B₁ is a carbon centre or nitrogen centre; and either:
(i) B₂ and B₃ together form a single bond between B₁ and the nitrogen atom to which R₁ is attached,
(ii) B₂ is a carbon centre or a nitrogen centre, and B₃ is a single bond between B₂ and the nitrogen atom to which R₁ is attached,
(iii) B₂ is a carbon centre and B₃ is a nitrogen centre, or
(iv) B₂ and B₃ are both carbon centres,
wherein, when B₂ is a carbon centre or nitrogen centre, B₁ is connected to B₂ via either a single or double bond;
wherein, when both B₂ and B₃ are present, B₂ and B₃ are connected via either a single or double bond;
wherein B₁ may, where possible, be optionally substituted with 1 or 2 substituents selected from alkyl, cycloalkyl, alkoxy, OH, CF₃, CN, and halo, or 1 oxo substituent, or 2 substituents which, together with B₁ to which they are both attached, form a 3-, 4-, or 5-membered non-aromatic monocyclic carbon-containing ring, optionally wherein one or two ring members are selected from N, S, and O;
wherein, when B₂ is a carbon centre, B₂ may be optionally substituted with 1 or 2 substituents selected from alkyl, cycloalkyl, alkoxy, OH, CF₃, CN, and halo, or 1 oxo substituent; and
wherein, when B₃ is a carbon centre, B₃ may be optionally substituted with 1 or 2 substituents selected from alkyl, cycloalkyl, alkoxy, OH, CF₃, CN, and halo, or 1 oxo substituent;
and either:
(i) B₄ is N(R₂), B₅ is N, and B₆ is C(R₃), and B₄ is connected to B₅ via a single bond and B₅ is connected to B₆ via a double bond;
(ii) B₄ is C(R₂), B₅ is N, and B₆ is N(R₃), and B₄ is connected to B₅ via a double bond and B₅ is connected to B₆ via a single bond;
(iii) B₄ is C(R₂), B₅ is C (H), and B₆ is N(R₃), and B₄ is connected to B₅ via a double bond and B₅ is connected to B₆ via a single bond; or
—(CH₂)₀₋₃—C(═O)—N(R₄)(R₅), —C(═O)—O—R₆, —(CH₂)₀₋₃—NR₁₄—C(═O) R₁₅, R₂ is selected from —(CH₂)₀₋₃—NR₃₄—S(═O)₂R₃₅, alkyl, cycloalkyl, heterocycloalkyl, alkoxy, —OH, —CF₃, halo, aryl, heteroaryl, —CH₂—O—R₁₆ and —CN;
wherein R₄ is H, alkyl, cycloalkyl, and heterocycloalkyl, and
R₅ is selected from alkyl, —OH, —S(═O)₂-(small alkyl), —(CH₂)₁₋₃—(C═O)—OR₃₃, —(CH₂)₀₋₃-cycloalkyl^{b}, —(CH₂)₀₋₃-heterocycloalkyl, —(CH₂)₁₋₃—(C═O)—N(R₃₆)(R₃₇), (CH(alkyl))-heterocycloalkyl, (CH₂)₀₋₃-polycycloalkyl, —(CH₂)₀₋₃-aryl, —(C(H)(phenyl))-alkyl, —(C(H)(heteroaryl))-alkyl, —(C(H)(alkyl))—(C═O)—OR₃₈, —(C(H)(heteroaryl))—(C═O)—OR₂₈, and —(CH₂)₀₋₃heteroaryl^{a}, —(C(H)(alkyl))—(C═O)—N(R₃₉)(R₄₀), —(C(H)(heteroaryl))—(C═O)—N(R₄₁)(R₄₂);
or wherein R₄ and R₅ together with the nitrogen atom to which they are attached form a carbon-containing 4-, 5-, 6-, or 7-membered heterocyclic ring which may be saturated or unsaturated with 1 or 2 double bonds, optionally containing one or two additional heteroatoms selected from N, O, and S,
wherein the ring may be optionally substituted with 1, 2, or 3 substituents selected from oxo, alkyl, alkoxy, OH, halo and CF_{3;}
R₂₈, R₃₃ R₃₆, R₃₇, R₃₈, R₃₉, R₄₀, R₄₁, and R₄₂ are each independently selected from H and small alkyl;
R₆ is selected from H, alkyl, —(CH₂)₀₋₃-cycloalkyl, —(CH₂)₀₋₃-heterocycloalkyl, —(CH(alkyl))-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, and —(C(H)(heteroaryl))-alkyl;
R₁₄ is selected from H, alkyl, cycloalkyl, and heterocycloalkyl;
R₁₅ is selected from H, alkyl, —(CH₂)₀₋₃-cycloalkyl, —(CH₂)₀₋₃-heterocycloalkyl, —(CH₂)₀₋₃-aryl, —(C(H)(phenyl))-alkyl, —(CH₂)₀₋₃heteroaryl^{a}, —(CH(alkyl))-heterocycloalkyl, and —(C(H)(heteroaryl))-alkyl;
R₁₆ is selected from H, alkyl, cycloalkyl, —(CH₂)₁₋₃—(C═O)—OH, heterocycloalkyl, and —(CH₂)₀₋₃heteroaryl;
R₃₄ is selected from H, alkyl, cycloalkyl, and heterocycloalkyl;
R₃₅ is selected from H, alkyl, —(CH₂)₀₋₃-cycloalkyl, —(CH₂)₀₋₃-heterocycloalkyl, —(CH(alkyl)-heterocycloalkyl, —(CH₂)₀₋₃-aryl, —(C(H)(phenyl))-alkyl, —(CH₂)₀₋₃heteroaryl^{a}, and —(C(H)(heteroaryl))-alkyl;
R₁ is:
(i) Formula (II)

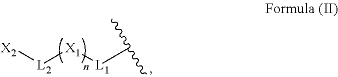

wherein:
n is 0 or 1;
L₁ is a linker selected from a single bond, —(CH₂)₁₋₃—, —(CH)(alkyl)-, and —C(alkyl)(alkyl)-;
X₁ is phenyl or a 5- or 6-membered carbon-containing aromatic ring containing one, two or three ring members that are selected from N, S, and O;
wherein X₁ may be optionally substituted with 1 or 2 substituents independently selected from alkyl, cycloalkyl, alkoxy, OH, O(cyclopropyl), CF₃, and halo;
L₂ is a linker selected from a single bond, —(CH₂)₁₋₃—, —(CH)(alkyl)-, —C(alkyl)(alkyl)-, —C(═O)—, and —SO₂—; and
X₂ is either:
(a) —NR₂OR₂₁,
wherein R₂₀ is selected from H, alkyl, and —(CH₂)₀₋₃-R_{22;}
wherein R₂₁ is selected from H, alkyl, and —(CH₂)₀₋₃-R₂₃;
wherein R₂₂ is selected from cycloalkyl, heterocycloalkyl, aryl, heteroaryl,

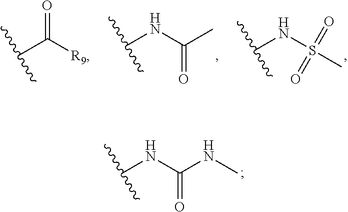

wherein R₂₃ is selected from cycloalkyl, heterocycloalkyl, aryl, heteroaryl,

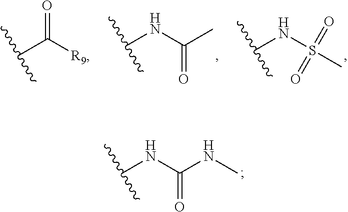

or
(b)₂-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O, and X₂ may be further optionally substituted with 1 or 2 substituents R₂₉ and R₃₀, wherein R₂₉ and R₃₀ are independently selected from alkyl, cycloalkyl, alkoxy, OH, oxo, CF₃, and halo; or,

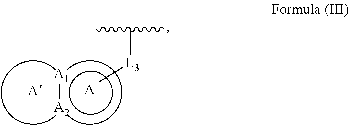

wherein:
L₃ is a linker selected from a single bond, —(CH₂)₁₋₃—, —(CH)(alkyl)-, and —C(alkyl)(alkyl)-;
A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, O(cyclopropyl), CF₃, and halo;
A′ is a non-aromatic carbon-containing monocyclic ring containing 5, 6, or 7, ring members, wherein at least one ring member is independently selected from N, S, and O;
wherein A′ may be optionally substituted with one R₇, wherein R₇ is selected from alkyl, alkoxy, and —(CH₂)₀₋₃-R₈,

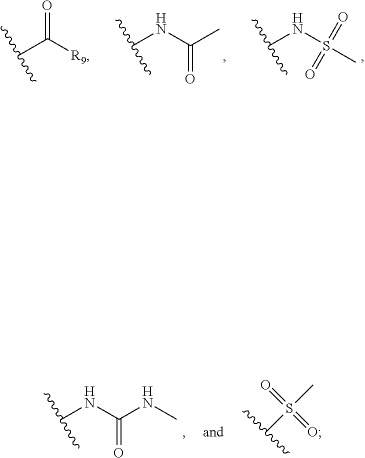

wherein R₈ is selected from cycloalkyl, heterocycloalkyl, aryl, heteroaryl, wherein R₉ is selected from H, alkyl, OH, N(R₃₁)(R₃₂), and alkoxy;
wherein R₃₁ is H or small alkyl;
wherein R₃₂ is selected from H, small alkyl, and —(CH₂)₀₋₃heteroaryl;
wherein A′ may be optionally substituted with 1, 2, or 3 further substituents R₁₀, R₁₁, and R₁₇, wherein R₁₀, R₁₁, and R₁₇, are independently selected from alkyl, cycloalkyl, alkoxy, OH, oxo, CF₃, and halo,
   or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O, or wherein A′ may be optionally substituted with 1 or 2 further substituents R₄₃ and R₄₄ independently selected from alkyl, cycloalkyl, alkoxy, OH, oxo, CF₃, and halo, and two different carbon ring members within A′ are connected by a bridging—CH₂-group; and
R₃ is phenyl, or a 5- or 6-membered carbon-containing aromatic ring containing one, two or three ring members that are selected from N, S, and O, wherein R₃ is substituted with one, two, or three substituents independently selected from small alkyl^{b},
small alkoxy, cyclopropyl, OH, NH₂, CF₃, halo, —C(═O)—NH₂, —C(═O)—N(H)(small alkyl), —C(═O)—N(small alkyl)(small alkyl), and CN; and alkyl is a linear saturated hydrocarbon having up to 10 carbon atoms (C₁-C₁₀) or a branched saturated hydrocarbon of between 3 and 10 carbon atoms (C₃-C₁₀); alkyl may optionally be substituted with 1, 2 or 3 substituents independently selected from cyclopropyl, (C₁-C₆)alkoxy, OH, —NR₁₂R₁₃, —C(═O)NR₁₂R₁₃, CN, CF₃, and halo;
small alkyl is a linear saturated hydrocarbon having up to 4 carbon atoms (C₁-C₄) or a branched saturated hydrocarbon of between 3 and 4 carbon atoms (C₃-C₄); small alkyl may optionally be substituted with 1 or 2 substituents independently selected from cyclopropyl, OH, CN, CF₃, NH₂, —C(═O)NH₂, and halo;
small alkyl^{b} is a linear saturated hydrocarbon having up to 4 carbon atoms (C₁-C₄) or a branched saturated hydrocarbon of between 3 and 4 carbon atoms (C₃-C₄); small alkyl may optionally be substituted with 1 or 2 substituents independently selected from cyclopropyl, OH, OMe, OEt, O'Pr, O″Pr, O(cyclopropyl), OCF₃, OCHF₂, CN, CF₃, NH₂, —C(═O) NH₂, and halo;
alkoxy is OCF₃, O(cyclopropyl), a linear O-linked hydrocarbon of between 1 and 6 carbon atoms (C₁-C₆), or a branched O-linked hydrocarbon of between 3 and 6 carbon atoms (C₃-C₆); alkoxy may optionally be substituted with 1 or 2 substituents independently selected from cyclopropyl, OH, CN, CF₃, and halo;
small alkoxy is OCF₃, O(cyclopropyl), a linear O-linked hydrocarbon of between 1 and 4 carbon atoms (C₁-C₄), or a branched O-linked hydrocarbon of between 3 and 4 carbon atoms (C₃-C₄); small alkoxy may optionally be substituted with 1 or 2 substituents independently selected from cyclopropyl, OH, CN, CF₃, and halo;
aryl is phenyl, biphenyl or naphthyl; aryl may be optionally substituted with 1, 2 or 3 substituents independently selected from alkyl, alkoxy, methylenedioxy, ethylenedioxy, OH, halo, CN, —(CH₂)₀₋₃—NR₁₂R₁₃, OCF₃ and CF_{3;}
polycycloalkyl is a bicyclic saturated hydrocarbon ring system of between 5 and 8 carbon atoms (C₅-C₈), wherein the bicyclic ring is bridged, or is adamantyl; polycycloalkyl may optionally be substituted with 1 or 2 substituents independently selected from alkyl, (C₁-C₆)alkoxy, OH, CN, CF₃, and halo;
cycloalkyl is a monocyclic saturated hydrocarbon ring of between 3 and 6 carbon atoms (C₃-C₆); cycloalkyl may optionally be substituted with 1 or 2 substituents independently selected from alkyl, (C₁-C₅)alkoxy, OH, CN, CF₃, and halo;
cycloalkyl^{a} is a monocyclic saturated hydrocarbon ring of between 3 and 6 carbon atoms (C₃-C₆); cycloalkyl may optionally be substituted with 1 or 2 substituents independently selected from alkyl, (C₁-C₅)alkoxy, OH, CN, CF₃, halo, and —(C═O)—OH;
cycloalkyl^{b} is a monocyclic saturated hydrocarbon ring of between 3 and 6 carbon atoms (C₃-C₆); cycloalkyl may optionally be substituted with 1 or 2 substituents independently selected from alkyl, (C₁-C₅)alkoxy, OH, NH₂, NH (small alkyl), CN, CF₃, halo, and —(C═O)—OH;
halo is F, Cl, Br, or I;
heteroaryl is a 5- or 6-membered carbon-containing aromatic ring containing one, two or three ring members that are selected from N, S, and O; wherein heteroaryl may be optionally substituted with 1, 2 or 3 substituents independently selected from alkyl, alkoxy, cyclopropyl, OH, halo, CN, and CF_{3;}
heteroaryl^{a} is a 5-, 6-, 9- or 10-membered mono- or bi-cyclic aromatic ring containing, where possible, 1, 2, 3 or 4 ring members independently selected from N, S, and O; wherein heteroaryl^{a} may be optionally substituted with 1, 2 or 3 substituents independently selected from alkyl, alkoxy, cyclopropyl, OH, halo, CN, and CF_{3;}
heterocycloalkyl is a non-aromatic carbon-containing mono- or bi-cyclic ring containing 3, 4, 5, 6, or 7 ring members, wherein at least one ring member is independently selected from N, S, and O; heterocycloalkyl may optionally be substituted with 1 or 2 substituents independently selected from alkyl, cyclopropyl, (C₁-C₆)alkoxy, OH, CN, CF₃, oxo, and halo; wherein when heterocycloalkyl is a bi-cyclic ring system, the bicyclic ring system is fused, bridged or spiro;
each R₁₂ and R₁₃ is independently selected from H, alkyl, cycloalkyl, and heterocycloalkyl;
and tautomers, isomers, stereoisomers (including enantiomers, diastereoisomers and racemic and scalemic mixtures thereof), deuterated analogues, and pharmaceutically acceptable salts and/or solvates thereof.

The invention further provides compounds of Formula (I),

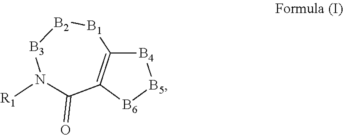

wherein:
B₁ is a carbon centre or nitrogen centre; and either:
(i) B₂ and B₃ together form a single bond between B₁ and the nitrogen atom to which R₁ is attached,
(ii) B₂ is a carbon centre or a nitrogen centre, and B₃ is a single bond between B₂ and the nitrogen atom to which R₁ is attached,
(iii) B₂ is a carbon centre and B₃ is a nitrogen centre, or
(iv) B₂ and B₃ are both carbon centres,
wherein, when B₂ is a carbon centre or nitrogen centre, B₁ is connected to B₂ via either a single or double bond;
wherein, when both B₂ and B₃ are present, B₂ and B₃ are connected via either a single or double bond;
wherein B₁ may, where possible, be optionally substituted with 1 or 2 substituents selected from alkyl, cycloalkyl, alkoxy, OH, CF₃, CN, and halo, or 1 oxo substituent, or 2 substituents which, together with B₁ to which they are both attached, form a 3-, 4-, or 5-membered non-aromatic monocyclic carbon-containing ring, optionally wherein one or two ring members are selected from N, S, and O;
wherein, when B₂ is a carbon centre, B₂ may be optionally substituted with 1 or 2 substituents selected from alkyl, cycloalkyl, alkoxy, OH, CF₃, CN, and halo, or 1 oxo substituent; and
wherein, when B₃ is a carbon centre, B₃ may be optionally substituted with 1 or 2 substituents selected from alkyl, cycloalkyl, alkoxy, OH, CF₃, CN, and halo, or 1 oxo substituent;
and either:
(i) B₄ is N(R₂), B₅ is N, and B₆ is C(R₃), and B₄ is connected to B₅ via a single bond and B₅ is connected to B₆ via a double bond;
(ii) B₄ is C(R₂), B₅ is N, and B₆ is N(R₃), and B₄ is connected to Bs via a double bond and B₅ is connected to B₆ via a single bond;
(iii) B₄ is C(R₂), B₅ is C (H), and B₆ is N(R₃), and B₄ is connected to B₅ via a double bond and B₅ is connected to B₆ via a single bond; or
R₂ is selected from H, —(CH₂)₀₋₃—C(═O)—N(R₄)(R₅), —C(═O)—O—R₆, —(CH₂)₀₋₃—NR₁₄—C(═O) R₁₅, —(CH₂)₀₋₃—NR₃₄—S(═O)₂R₃₅, alkyl, cycloalkyl, heterocycloalkyl, alkoxy, —OH, —CF₃, halo, aryl, heteroaryl, —CH₂—O—R₁₆ and —CN;
wherein R₄ is H, alkyl, cycloalkyl, and heterocycloalkyl, and
R₅ is selected from alkyl, —OH, —S(═O)₂-(small alkyl), —(CH₂)₁₋₃—(C═O)—OR₃₃, —(CH₂)₁₋₃—(C═O)—N(R₃₆)(R₃₇), —(CH₂)₀₋₃-cycloalkyl^{a}, —(CH₂)₀₋₃-heterocycloalkyl, —(CH₂)₀₋₃-polycycloalkyl, —(CH₂)₀₋₃-aryl, —(C(H)(phenyl))-alkyl, —(CH(alkyl))-heterocycloalkyl, —(CH₂)₀₋₃heteroaryl^{a}, —(C(H)(heteroaryl))-alkyl, —(C(H)(alkyl))—(C═O)—OR₃₈, —(C(H)(alkyl))—(C═O)—N(R₃₉)(R₄₀), —(C(H)(heteroaryl))—(C═O)—OR₂₈, and —(C(H)(heteroaryl))—(C═O)—N(R₄₁)(R₄₂);
or wherein R₄ and R₅ together with the nitrogen atom to which they are attached form a carbon-containing 4-, 5-, 6-, or 7-membered heterocyclic ring which may be saturated or unsaturated with 1 or 2 double bonds, optionally containing one or two additional heteroatoms selected from N, O, and S, wherein the ring may be optionally substituted with 1, 2, or 3 substituents selected from oxo, alkyl, alkoxy, OH, halo and CF₃;
R₂₈, R₃₃ R₃₆, R₃₇, R₃₈, R₃₉, R₄₀, R₄₁, and R₄₂ are each independently selected from H and small alkyl; R₆ is selected from H, alkyl, —(CH₂)₀₋₃-cycloalkyl, —(CH₂)₀₋₃-heterocycloalkyl, —(CH(alkyl))-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, and —(C(H)(heteroaryl))-alkyl;
R₁₄ is selected from H, alkyl, cycloalkyl, and heterocycloalkyl;
R₁₅ is selected from H, alkyl, —(CH₂)₀₋₃-cycloalkyl, —(CH₂)₀₋₃-heterocycloalkyl, —(CH(alkyl))-heterocycloalkyl, —(CH₂)₀₋₃-aryl, —(C(H)(phenyl))-alkyl, —(CH₂)₀₋₃heteroaryl^{a}, and —(C(H)(heteroaryl))-alkyl;
R₁₆ is selected from H, alkyl, cycloalkyl, —(CH₂)₁₋₃—(C═O)—OH, heterocycloalkyl, and —(CH₂)₀₋₃heteroaryl; R₃₄ is selected from H, alkyl, cycloalkyl, and heterocycloalkyl;
R₃₅ is selected from H, alkyl, —(CH₂)₀₋₃-cycloalkyl, —(CH₂)₀₋₃-heterocycloalkyl, —(CH(alkyl))-heterocycloalkyl, —(CH₂)₀₋₃-aryl, —(C(H)(phenyl))-alkyl, —(CH₂)₀₋₃heteroaryl^{a}, and —(C(H)(heteroaryl))-alkyl;
R₁ is:
(i) Formula (II), <CWU-Call number

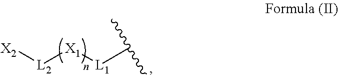

wherein:
n is 0 or 1;
L₁ is a linker selected from a single bond, —(CH₂)₁₋₃—, —(CH)(alkyl)-, and —C(alkyl)(alkyl)-;
X₁ is phenyl or a 5- or 6-membered carbon-containing aromatic ring containing one, two or three ring members that are selected from N, S, and O;
wherein X₁ may be optionally substituted with 1 or 2 substituents independently selected from alkyl, cycloalkyl, alkoxy, OH, CF₃, and halo;
L₂ is a linker selected from a single bond, —(CH₂)₁₋₃—, —(CH)(alkyl)-, —C(alkyl)(alkyl)-, —C(═O)—, and —SO₂—; and

X₂ is either:
wherein R₂₀ is selected from H, alkyl, and —(CH₂)₀₋₃-R_{22;}
wherein R₂₁ is selected from H, alkyl, and —(CH₂)₀₋₃-R_{23;}
(a) —NR₂OR₂₁,

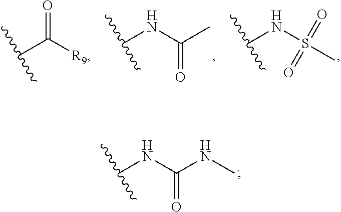

wherein R₂₂ is selected from cycloalkyl, heterocycloalkyl, aryl, heteroaryl,

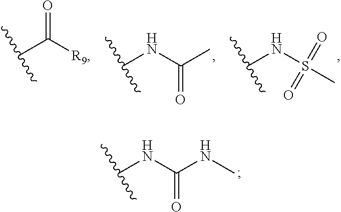

wherein R₂₃ is selected from cycloalkyl, heterocycloalkyl, aryl, heteroaryl, or
(b)₂-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O, and X₂ may be further optionally substituted with 1 or 2 substituents R₂₉ and R₃₀, wherein R₂₉ and R₃₀ are independently selected from alkyl, cycloalkyl, alkoxy, OH, oxo, CF₃, and halo; or,
(ii) Formula (III) comprising an aromatic ring A fused to a non-aromatic ring A′,

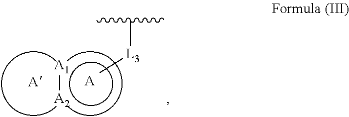

wherein:
L₃ is a linker selected from a single bond, —(CH₂)₁₋₃—, —(CH)(alkyl)-, and —C(alkyl)(alkyl)-; A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R₇ is selected from alkyl, alkoxy, and —(CH₂)₀₋₃—R₈,

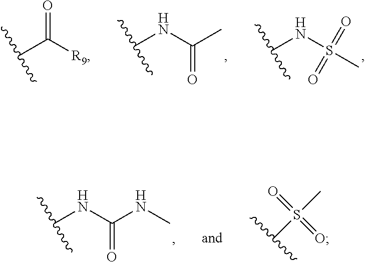

wherein R₈ is selected from cycloalkyl, heterocycloalkyl, aryl, heteroaryl,
wherein R₉ is selected from H, alkyl, OH, N(R₃₁)(R₃₂), and alkoxy;
wherein R₃₁ is H or small alkyl;
wherein R₃₂ is selected from H, small alkyl, and —(CH₂)₀₋₃heteroaryl;
wherein A′ may be optionally substituted with 1, 2, or 3 further substituents R₁₀, R₁₁, and R₁₇, wherein R₁₀, R₁₁, and R₁₇ are independently selected from alkyl, cycloalkyl, alkoxy, OH, oxo, CF₃, and halo, or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O; and

R₃ is phenyl, or a 5- or 6-membered carbon-containing aromatic ring containing one, two or three ring members that are selected from N, S, and O, wherein R₃ is substituted with one or two substituents independently selected from small alkyl, small alkoxy, cyclopropyl, OH, CF₃, halo, —C(═O)—NH₂, —C(═O)—N(H)(small alkyl), —C(═O)—N(small alkyl)(small alkyl), and CN; and alkyl is a linear saturated hydrocarbon having up to 10 carbon atoms (C₁-C₁₀) or a branched saturated hydrocarbon of between 3 and 10 carbon atoms (C₃-C₁₀); alkyl may optionally be substituted with 1, 2 or 3 substituents independently selected from cyclopropyl, (C₁-C₆)alkoxy, OH, —NR₁₂R₁₃, —C(═O) NR₁₂R₁₃, CN,

CF₃, and halo;

small alkyl is a linear saturated hydrocarbon having up to 4 carbon atoms (C₁-C₄) or a branched saturated hydrocarbon of between 3 and 4 carbon atoms (C₃-C₄); small alkyl may optionally be substituted with 1 or 2 substituents independently selected from cyclopropyl, OH, CN, CF₃, NH₂, —C(═O) NH₂, and halo;

alkoxy is OCF₃, a linear O-linked hydrocarbon of between 1 and 6 carbon atoms (C₁-C₆), or a branched O-linked hydrocarbon of between 3 and 6 carbon atoms (C₃-C₆); alkoxy may optionally be substituted with 1 or 2 substituents independently selected from cyclopropyl, OH, CN, CF₃, and halo;

small alkoxy is OCF₃, a linear O-linked hydrocarbon of between 1 and 4 carbon atoms (C₁-C₄), or a branched O-linked hydrocarbon of between 3 and 4 carbon atoms (C₃-C₄); small alkoxy may optionally be substituted with 1 or 2 substituents independently selected from cyclopropyl, OH, CN, CF₃, and halo;

aryl is phenyl, biphenyl or naphthyl; aryl may be optionally substituted with 1, 2 or 3 substituents independently selected from alkyl, alkoxy, methylenedioxy, ethylenedioxy, OH, halo, CN, —(CH₂)₀₋₃—NR₁₂R₁₃, OCF₃ and CF_{3;}

polycycloalkyl is a bicyclic saturated hydrocarbon ring system of between 5 and 8 carbon atoms (C₅-C₈), wherein the bicyclic ring is bridged, or is adamantyl; polycycloalkyl may optionally be substituted with 1 or 2 substituents independently selected from alkyl, (C₁-C₆)alkoxy, OH, CN, CF₃, and halo;

cycloalkyl is a monocyclic saturated hydrocarbon ring of between 3 and 6 carbon atoms (C₃-C₆); cycloalkyl may optionally be substituted with 1 or 2 substituents independently selected from alkyl, (C₁-C₅)alkoxy, OH, CN, CF₃, and halo;

cycloalkyl^{a} is a monocyclic saturated hydrocarbon ring of between 3 and 6 carbon atoms (C₃-C₆); cycloalkyl may optionally be substituted with 1 or 2 substituents independently selected from alkyl, (C₁-C₅)alkoxy,

OH, CN, CF₃, halo, and —(C═O)—OH;

halo is F, Cl, Br, or I;

heteroaryl is a 5- or 6-membered carbon-containing aromatic ring containing one, two or three ring members that are selected from N, S, and O; wherein heteroaryl may be optionally substituted with 1, 2 or 3 substituents independently selected from alkyl, alkoxy, cyclopropyl, OH, halo, CN, and CF_{3;}

heteroaryl^{a} is a 5-, 6-, 9- or 10-membered mono- or bi-cyclic aromatic ring containing, where possible, 1, 2, 3 or 4 ring members independently selected from N, S, and O; wherein heteroaryl^{a} may be optionally substituted with 1, 2 or 3 substituents independently selected from alkyl, alkoxy, cyclopropyl, OH, halo, CN, and CF_{3;}

heterocycloalkyl is a non-aromatic carbon-containing mono- or bi-cyclic ring containing 3, 4, 5, 6, or 7 ring members, wherein at least one ring member is independently selected from N, S, and O; heterocycloalkyl may optionally be substituted with 1 or 2 substituents independently selected from alkyl, cyclopropyl, (C₁-C₆)alkoxy, OH, CN, CF₃, oxo, and halo; wherein when heterocycloalkyl is a bi-cyclic ring system, the bicyclic ring system is fused, bridged or spiro;

each R₁₂ and R₁₃ is independently selected from H, alkyl, cycloalkyl, and heterocycloalkyl;

and tautomers, isomers, stereoisomers (including enantiomers, diastereoisomers and racemic and scalemic mixtures thereof), deuterated analogues, and pharmaceutically acceptable salts and/or solvates thereof.

The invention further provides compounds of Formula (I),

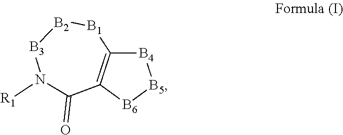

wherein:
B₁ is a carbon centre or nitrogen centre; and either:
(i) B₂ and B₃ together form a single bond between B₁ and the nitrogen atom to which R₁ is attached,
(ii) B₂ is a carbon centre or a nitrogen centre, and B₃ is a single bond between B₂ and the nitrogen atom to which R₁ is attached,
(iii) B₂ is a carbon centre and B₃ is a nitrogen centre, or
(iv) B₂ and B₃ are both carbon centres, wherein, when B₂ is a carbon centre or nitrogen centre, B₁ is connected to B₂ via either a single or double bond;

wherein, when both B₂ and B₃ are present, B₂ and B₃ are connected via either a single or double bond;
wherein B₁ may, where possible, be optionally substituted with 1 or 2 substituents selected from alkyl, cycloalkyl, alkoxy, OH, CF₃, CN, and halo, or 1 oxo substituent, or 2 substituents which, together with B₁ to which they are both attached, form a 3-, 4-, or 5-membered non-aromatic monocyclic carbon-containing ring, optionally wherein one or two ring members are selected from N, S, and O;

wherein, when B₂ is a carbon centre, B₂ may be optionally substituted with 1 or 2 substituents selected from alkyl, cycloalkyl, alkoxy, OH, CF₃, CN, and halo, or 1 oxo substituent; and

wherein, when B₃ is a carbon centre, B₃ may be optionally substituted with 1 or 2 substituents selected from alkyl, cycloalkyl, alkoxy, OH, CF₃, CN, and halo, or 1 oxo substituent;

and either:
(i) B₄ is N(R₂), B₅ is N, and B₆ is C(R₃), and B₄ is connected to B₅ via a single bond and B₅ is connected to B₆ via a double bond;
(ii) B₄ is C(R₂), B₅ is N, and B₆ is N(R₃), and B₄ is connected to B₅ via a double bond and B₅ is connected to B₆ via a single bond;
(iii) B₄ is C(R₂), B₅ is C (H), and B₆ is N(R₃), and B₄ is connected to B₅ via a double bond and B₅ is connected to B₆ via a single bond; or
R₂ is selected from H, —(CH₂)₀₋₃—C(═O)—N(R₄)(R₅), —C(═O)—O—R₆, —(CH₂)₀₋₃—NR₁₄—C(═O) R₁₅, —(CH₂)₀₋₃—NR₃₄—S(═O)₂R₃₅, alkyl, cycloalkyl, heterocycloalkyl, alkoxy, —OH, —CF₃, halo, aryl, heteroaryl, —CH₂-O—R₁₆ and —CN;
wherein R₄ is H, alkyl, cycloalkyl, and heterocycloalkyl, and
R₅ is selected from alkyl, —(CH₂)₀₋₃-cycloalkyl, —(CH₂)₀₋₃-heterocycloalkyl, —(CH(alkyl))-heterocycloalkyl, —(CH₂)₀₋₃-polycycloalkyl, —(CH₂)₀₋₃-aryl, —(C(H)(phenyl))-alkyl, —(CH₂)₀₋₃heteroaryl^{a}, and —(C(H)(heteroaryl))-alkyl;
or wherein R₄ and R₅ together with the nitrogen atom to which they are attached form a carbon-containing 4-, 5-, 6-, or 7-membered heterocyclic ring which may be saturated or unsaturated with 1 or 2 double bonds, optionally containing one or two additional heteroatoms selected from N, O, and S, wherein the ring may be optionally substituted with 1, 2, or 3 substituents selected from oxo, alkyl, alkoxy, OH, halo and CF_{3;}
R₆ is selected from H, alkyl, —(CH₂)₀₋₃-cycloalkyl, —(CH₂)₀₋₃-heterocycloalkyl, —(CH(alkyl))-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, and —(C(H)(heteroaryl))-alkyl; R₁₄ is selected from H, alkyl, cycloalkyl, and heterocycloalkyl;
R₁₅ is selected from H, alkyl, —(CH₂)₀₋₃-cycloalkyl, —(CH₂)₀₋₃-heterocycloalkyl, —(CH(alkyl)-heterocycloalkyl, —(CH₂)₀₋₃-aryl, —(C(H)(phenyl))-alkyl, —(CH₂)₀₋₃heteroaryl^{a}, and —(C(H)(heteroaryl))-alkyl; R₁₆ is selected from H, alkyl, cycloalkyl, and heterocycloalkyl; R₃₄ is selected from H, alkyl, cycloalkyl, and heterocycloalkyl; R₃₅ is selected from H, alkyl, —(CH₂)₀₋₃-cycloalkyl, —(CH₂)₀₋₃-heterocycloalkyl, —(CH(alkyl))-heterocycloalkyl, —(CH₂)₀₋₃-aryl, —(C(H)(phenyl))-alkyl, —(CH₂)₀₋₃heteroaryl^{a}, and —(C(H)(heteroaryl))-alkyl;
R₁ is:

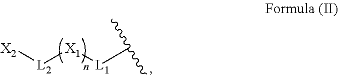

wherein:
n is 0 or 1;
L₁ is a linker selected from a single bond, —(CH₂)₁₋₃—, —(CH)(alkyl)-, and —C(alkyl)(alkyl)-; X₁ is phenyl or a 5- or 6-membered carbon-containing aromatic ring containing one, two or three ring members that are selected from N, S, and O;
wherein X₁ may be optionally substituted with 1 or 2 substituents independently selected from alkyl, cycloalkyl, alkoxy, OH, CF₃, and halo;
L₂ is a linker selected from a single bond, —(CH₂)₁₋₃—, —(CH)(alkyl)-, —C(alkyl)(alkyl)-, —C(═O)—, and —SO₂—; and

X₂ is either:
(a) —NR₂OR₂₁ wherein R₂₀ is selected from H, alkyl, and —(CH₂)₀₋₃-R_{22;}
wherein R₂₁ is selected from H, alkyl, and —(CH₂)₀₋₃-R₂₃;

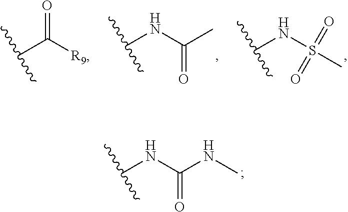

wherein R₂₂ is selected from cycloalkyl, heterocycloalkyl, aryl, heteroaryl,

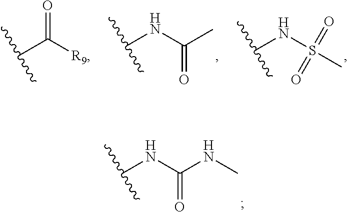

wherein R₂₃ is selected from cycloalkyl, heterocycloalkyl, aryl, heteroaryl, or
(b)₂-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected alkyl, cycloalkyl, alkoxy, OH, oxo, CF₃, and from halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O; or,

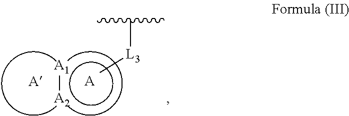

wherein:
L₃ is a linker selected from a single bond, —(CH₂)₁₋₃—, —(CH)(alkyl)-, and —C(alkyl)(alkyl)-; A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R₇ is selected from alkyl, alkoxy, and —(CH₂)₀₋₃-R₈,

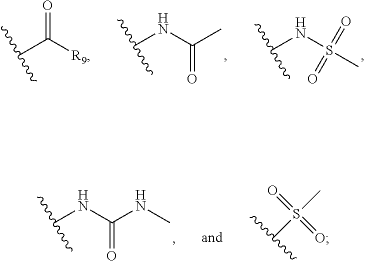

wherein R₈ is selected from cycloalkyl, heterocycloalkyl, aryl, heteroaryl, wherein R₉ is selected from H, alkyl, OH, NH₂, and alkoxy;
wherein A′ may be optionally substituted with 1, 2, or 3 further substituents R₁₀, R₁₁, and R₁₇, wherein R₁₀, R₁₁, and R₁₇ are independently selected from alkyl, cycloalkyl, alkoxy, OH, oxo, CF₃, and halo, or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O; and

R₃ is phenyl, or a 5- or 6-membered carbon-containing aromatic ring containing one, two or three ring members that are selected from N, S, and O, wherein R₃ is substituted with one or two substituents independently selected from small alkyl, small alkoxy, cyclopropyl, OH, CF₃, halo, —C(═O)—NH₂, —C(═O)—N(H)(small alkyl), —C(═O)—N(small alkyl)(small alkyl), and CN; and alkyl is a linear saturated hydrocarbon having up to 10 carbon atoms (C₁-C₁₀) or a branched saturated hydrocarbon of between 3 and 10 carbon atoms (C₃-C₁₀); alkyl may optionally be substituted with 1, 2 or 3 substituents independently selected from cyclopropyl, (C₁-C₅)alkoxy, OH, —NR₁₂R₁₃, —C(═O) NR₁₂R₁₃, CN, CF₃, and halo;

small alkyl is a linear saturated hydrocarbon having up to 4 carbon atoms (C₁-C₄) or a branched saturated hydrocarbon of between 3 and 4 carbon atoms (C₃-C₄); small alkyl may optionally be substituted with 1 or 2 substituents independently selected from cyclopropyl, OH, CN, CF₃, NH₂, —C(═O) NH₂, and halo;

alkoxy is OCF₃, a linear O-linked hydrocarbon of between 1 and 6 carbon atoms (C₁-C₅), or a branched

O-linked hydrocarbon of between 3 and 6 carbon atoms (C₃-C₆); alkoxy may optionally be substituted with 1 or 2 substituents independently selected from cyclopropyl, OH, CN, CF₃, and halo;

small alkoxy is OCF₃, a linear O-linked hydrocarbon of between 1 and 4 carbon atoms (C₁-C₄), or a branched O-linked hydrocarbon of between 3 and 4 carbon atoms (C₃-C₄); small alkoxy may optionally be substituted with 1 or 2 substituents independently selected from cyclopropyl, OH, CN, CF₃, and halo; aryl is phenyl, biphenyl or naphthyl; aryl may be optionally substituted with 1, 2 or 3 substituents independently selected from alkyl, alkoxy, methylenedioxy, ethylenedioxy, OH, halo, CN, —(CH₂)₀₋₃—NR₁₂R₁₃, OCF₃ and CF_{3;}
polycycloalkyl is a bicyclic saturated hydrocarbon ring system of between 5 and 8 carbon atoms (C₅-C₈), wherein the bicyclic ring is bridged, or is adamantyl; polycycloalkyl may optionally be substituted with 1 or 2 substituents independently selected from alkyl, (C₁-C₆)alkoxy, OH, CN, CF₃, and halo;

cycloalkyl is a monocyclic saturated hydrocarbon ring of between 3 and 6 carbon atoms (C₃-C₆); cycloalkyl may optionally be substituted with 1 or 2 substituents independently selected from alkyl, (C₁-C₆)alkoxy, OH, CN, CF₃, and halo;

halo is F, Cl, Br, or I;

heteroaryl is a 5- or 6-membered carbon-containing aromatic ring containing one, two or three ring members that are selected from N, S, and O; wherein heteroaryl may be optionally substituted with 1, 2 or 3 substituents independently selected from alkyl, alkoxy, cyclopropyl, OH, halo, CN, and CF_{3;}

heteroaryl^{a} is a 5-, 6-, 9- or 10-membered mono- or bi-cyclic aromatic ring containing, where possible, 1, 2, 3 or 4 ring members independently selected from N, S, and O; wherein heteroaryl^{a} may be optionally substituted with 1, 2 or 3 substituents independently selected from alkyl, alkoxy, cyclopropyl, OH, halo, CN, and CF_{3;}

heterocycloalkyl is a non-aromatic carbon-containing mono- or bi-cyclic ring containing 3, 4, 5, 6, or 7 ring members, wherein at least one ring member is independently selected from N, S, and O; heterocycloalkyl may optionally be substituted with 1 or 2 substituents independently selected from alkyl, cyclopropyl, (C₁-C₆)alkoxy, OH, CN, CF₃, oxo, and halo; wherein when heterocycloalkyl is a bi-cyclic ring system, the bicyclic ring system is fused, bridged or spiro;

each R₁₂ and R₁₃ is independently selected from H, alkyl, cycloalkyl, and heterocycloalkyl;

and tautomers, isomers, stereoisomers (including enantiomers, diastereoisomers and racemic and scalemic mixtures thereof), deuterated analogues, and pharmaceutically acceptable salts and/or solvates thereof.

The invention also provides compounds of Formula (I),

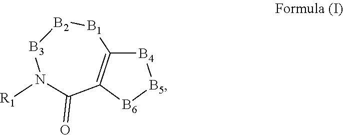

wherein:
B₁ is a carbon centre or nitrogen centre; and either:
(i) B₂ and B₃ together form a single bond between B₁ and the nitrogen atom to which R₁ is attached,
(ii) B₂ is a carbon centre or a nitrogen centre, and B₃ is a single bond between B₂ and the nitrogen atom to which R₁ is attached,
(iii) B₂ is a carbon centre and B₃ is a nitrogen centre, or
(iv) B₂ and B₃ are both carbon centres, wherein, when B₂ is a carbon centre or nitrogen centre, B₁ is connected to B₂ via either a single or double bond;

wherein, when both B₂ and B₃ are present, B₂ and B₃ are connected via either a single or double bond;
wherein B₁ may, where possible, be optionally substituted with 1 or 2 substituents selected from alkyl, cycloalkyl, alkoxy, OH, CF₃, CN, and halo, or 1 oxo substituent, or 2 substituents which, together with B₁ to which they are both attached, form a 3-, 4-, or 5-membered non-aromatic monocyclic carbon-containing ring, optionally wherein one or two ring members are selected from N, S, and O; wherein, when B₂ is a carbon centre, B₂ may be optionally substituted with 1 or 2 substituents selected from alkyl, cycloalkyl, alkoxy, OH, CF₃, CN, and halo, o 1 oxo substituent; and wherein, when B₃ is a carbon centre, B₃ may be optionally substituted with 1 or 2 substituents selected from alkyl, cycloalkyl, alkoxy, OH, CF₃, CN, and halo, or 1 oxo substituent;

and either:
(i) B₄ is N(R₂), B₅ is N, and B₆ is C(R₃), and B₄ is connected to Bs via a single bond and B₅ is connected to B₆ via a double bond;
(ii) B₄ is C(R₂), B₅ is N, and B₆ is N(R₃), and B₄ is connected to B₅ via a double bond and B₅ is connected to B₆ via a single bond;
(iii) B₄ is C(R₂), B₅ is C (H), and B₆ is N(R₃), and B₄ is connected to B₅ via a double bond and B₅ is connected to B₆ via a single bond; or
R₂ is selected from H, —(CH₂)₀₋₃—C(═O)—N(R₄)(R₅), —C(═O)—O—R₆, —(CH₂)₀₋₃—NR₁₄—C(═O) R₁₅, alkyl, cycloalkyl, heterocycloalkyl, alkoxy, —OH, —CF₃, halo, aryl, heteroaryl, —CH₂—O—R₁₆ and —CN;
wherein R₄ is H, alkyl, cycloalkyl, and heterocycloalkyl, and
R₅ is selected from alkyl, —(CH₂)₀₋₃-cycloalkyl, —(CH₂)₀₋₃-heterocycloalkyl, —(CH₂)₀₋₃-polycycloalkyl, —(CH₂)₀₋₃-aryl, —(C(H)(phenyl))-alkyl, —(CH₂)₀₋₃heteroaryl^{a}, and —(C(H)(heteroaryl))-alkyl;
or wherein R₄ and R₅ together with the nitrogen atom to which they are attached form a carbon-containing 4-, 5-, 6-, or 7-membered heterocyclic ring which may be saturated or unsaturated with 1 or 2 double bonds, optionally containing one or two additional heteroatoms selected from N, O, and S, wherein the ring may be optionally substituted with 1, 2, or 3 substituents selected from oxo, alkyl, alkoxy,

OH, halo and CF_{3;}
R₆ —(CH₂)₀₋₃-cycloalkyl, H, alkyl, —(C(H)(phenyl)-alkyl, —(CH₂)₀₋₃heteroaryl, and —(C(H)(heteroaryl)-alkyl;

is selected from —(CH₂)₀₋₃-heterocycloalkyl, —(CH₂)₀₋₃-polycycloalkyl, —(CH₂)₀₋₃-aryl,
R₁₄ is selected from H, alkyl, cycloalkyl, and heterocycloalkyl;
R₁₅ is selected from H, alkyl, —(CH₂)₀₋₃-cycloalkyl, —(CH₂)₀₋₃-heterocycloalkyl, —(CH₂)₀₋₃-aryl, —(C(H)(phenyl))-alkyl, —(CH₂)₀₋₃heteroaryl^{a}, and —(C(H)(heteroaryl))-alkyl;
R₁₆ is selected from H, alkyl, cycloalkyl, and heterocycloalkyl;
R₁ is:
(i) Formula (II),

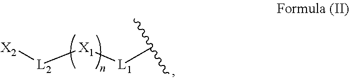

wherein:
n is 0 or 1;
L₁ is a linker selected from a single bond, —(CH₂)₁₋₃—, —(CH)(alkyl)-, and —C(alkyl)(alkyl);
X₁ is phenyl or a 5- or 6-membered carbon-containing aromatic ring containing one, two or three ring members that are selected from N, S, and O;
wherein X₁ may be optionally substituted with 1 or 2 substituents independently selected from alkyl, cycloalkyl, alkoxy, OH, CF₃, and halo;
L₂ is a linker selected from a single bond, —(CH₂)₁₋₃—, —(CH)(alkyl)-, —C(alkyl)(alkyl)-, —C(═O)—, and —SO₂—; and

X₂ is either:
(a) —NR₂OR₂₁, wherein R₂₀ is selected from H, alkyl, and —(CH₂)₀₋₃-R_{22;}
wherein R₂₁ is selected from H, alkyl, and —(CH₂)₀₋₃-R₂₃;

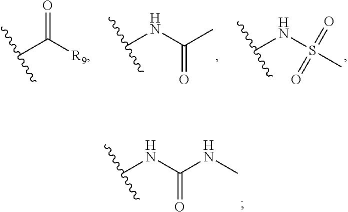

wherein R₂₂ is selected from cycloalkyl, heterocycloalkyl, aryl, heteroaryl,

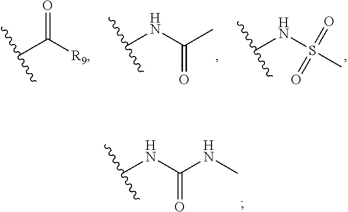

wherein R₂₃ is selected from cycloalkyl, heterocycloalkyl, aryl, heteroaryl, or
(b)₂-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected from alkyl, cycloalkyl, alkoxy, OH, Oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O; or,
(ii) Formula (III) comprising an aromatic ring A fused to a non-aromatic ring A′,

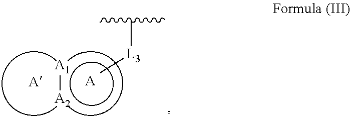

wherein:
L₃ is a linker selected from a single bond, —(CH₂)₁₋₃—, —(CH)(alkyl)-, and —C(alkyl)(alkyl)-; A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R₇ is selected from alkyl, alkoxy, and —(CH₂)₀₋₃-R₈,
wherein R₈ is selected from cycloalkyl, heterocycloalkyl, aryl, heteroaryl,

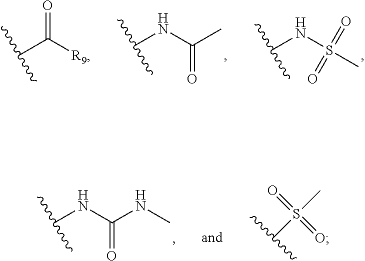

wherein R₉ is selected from H, alkyl, OH, NH₂, and alkoxy;
wherein A′ may be optionally substituted with 1 or 2 further substituents R₁₀ and R₁₁, wherein R₁₀ and R₁₁ are independently selected from alkyl, cycloalkyl, alkoxy, OH, oxo, CF₃, and halo, or wherein A′ may be optionally substituted with 1 further substituent R₂₄ selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O; and

R₃ is phenyl, or a 5- or 6-membered carbon-containing aromatic ring containing one, two or three ring members that and are selected from N, S, O, wherein R₃ is substituted with one or two substituents independently selected from small alkyl, small alkoxy, cyclopropyl, OH, CF₃, halo, —C(═O)—NH₂, —C(═O)—N(H)(small alkyl), —C(═O)—N(small alkyl)(small alkyl), and CN; and alkyl is a linear saturated hydrocarbon having up to 10 carbon atoms (C₁-C₁₀) or a branched saturated hydrocarbon of between 3 and 10 carbon atoms (C₃-C₁₀); alkyl may optionally be substituted with 1, 2 or 3 substituents independently selected from cyclopropyl, (C₁-C₅)alkoxy, OH, —NR₁₂R₁₃, —C(═O) NR₁₂R₁₃, CN, CF₃, and halo;

small alkyl is a linear saturated hydrocarbon having up to 4 carbon atoms (C₁-C₄) or a branched saturated hydrocarbon of between 3 and 4 carbon atoms (C₃-C₄); small alkyl may optionally be substituted with 1 or 2 substituents independently selected from cyclopropyl, OH, CN, CF₃, NH₂, —C(═O) NH₂, and halo;

alkoxy is OCF₃, a linear O-linked hydrocarbon of between 1 and 6 carbon atoms (C₁-C₆), or a branched O-linked hydrocarbon of between 3 and 6 carbon atoms (C₃-C₆); alkoxy may optionally be substituted with 1 or 2 substituents independently selected from cyclopropyl, OH, CN, CF₃, and halo;

small alkoxy is OCF₃, a linear O-linked hydrocarbon of between 1 and 4 carbon atoms (C₁-C₄), or a branched O-linked hydrocarbon of between 3 and 4 carbon atoms (C₃-C₄); small alkoxy may optionally be substituted with 1 or 2 substituents independently selected from cyclopropyl, OH, CN, CF₃, and halo;

aryl is phenyl, biphenyl or naphthyl; aryl may be optionally substituted with 1, 2 or 3 substituents independently selected from alkyl, alkoxy, methylenedioxy, ethylenedioxy, OH, halo, CN, —(CH₂)₀₋₃—NR₁₂R₁₃, OCF₃ and CF_{3;}

polycycloalkyl is a bicyclic saturated hydrocarbon ring system of between 5 and 8 carbon atoms (C₅-C₈), wherein the bicyclic ring is bridged, or is adamantyl; polycycloalkyl may optionally be substituted with 1 or 2 substituents independently selected from alkyl, (C₁-C₆)alkoxy, OH, CN, CF₃, and halo;

cycloalkyl is a monocyclic saturated hydrocarbon ring of between 3 and 6 carbon atoms (C₃-Cs); cycloalkyl may optionally be substituted with 1 or 2 substituents independently selected from alkyl, (C₁-C₅)alkoxy,

OH, CN, CF₃, and halo;

halo is F, Cl, Br, or I;

heteroaryl is a 5- or 6-membered carbon-containing aromatic ring containing one, two or three ring members that are selected from N, S, and O; wherein heteroaryl may be optionally substituted with 1, 2 or 3 substituents independently selected from alkyl, alkoxy, cyclopropyl, OH, halo, CN, and CF_{3;}

heteroaryl^{a} is a 5-, 6-, 9- or 10-membered mono- or bi-cyclic aromatic ring containing, where possible, 1, 2, 3 or 4 ring members independently selected from N, S, and O; wherein heteroaryl^{a} may be optionally substituted with 1, 2 or 3 substituents independently selected from alkyl, alkoxy, cyclopropyl, OH, halo, CN, and CF_{3;}

heterocycloalkyl is a non-aromatic carbon-containing mono- or bi-cyclic ring containing 3, 4, 5, 6, or 7 ring members, wherein at least one ring member is independently selected from N, S, and O; heterocycloalkyl may optionally be substituted with 1 or 2 substituents independently selected from alkyl, cyclopropyl, (C₁-C₆)alkoxy, OH, CN, CF₃, oxo, and halo; wherein when heterocycloalkyl is a bi-cyclic ring system, the bicyclic ring system is fused, bridged or spiro;

each R₁₂ and R₁₃ is independently selected from H, alkyl, cycloalkyl, and heterocycloalkyl;

and tautomers, isomers, stereoisomers (including enantiomers, diastereoisomers and racemic and scalemic mixtures thereof), deuterated analogues, and pharmaceutically acceptable salts and/or solvates thereof.

The invention also provides compounds of Formula (I),

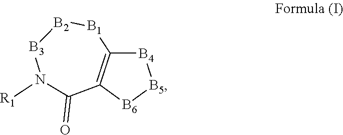

wherein:
B₁ is a carbon centre or nitrogen centre; and either:
(i) B₂ and B₃ together form a single bond between B₁ and the nitrogen atom to which R₁ is attached,
(ii) B₂ is a carbon centre or a nitrogen centre, and B₃ is a single bond between B₂ and the nitrogen atom to which R₁ is attached,
(iii) B₂ is a carbon centre and B₃ is a nitrogen centre, or
(iv) B₂ and B₃ are both carbon centres, wherein, when B₂ is a carbon centre or nitrogen centre, B₁ is connected to B₂ via either a single or double bond;

wherein, when both B₂ and B₃ are present, B₂ and B₃ are connected via either a single or double bond;
wherein B₁ may, where possible, be optionally substituted with 1 or 2 substituents selected from alkyl, cycloalkyl, alkoxy, OH, CF₃, CN, and halo, or 1 oxo substituent, or 2 substituents which, together with B₁ to which they are both attached, form a 3-, 4-, or 5-membered non-aromatic monocyclic carbon-containing ring, optionally wherein one or two ring members are selected from N, S, and O;

wherein, when B₂ is a carbon centre, B₂ may be optionally substituted with 1 or 2 substituents selected from alkyl, cycloalkyl, alkoxy, OH, CF₃, CN, and halo, or 1 oxo substituent; and

wherein, when B₃ is a carbon centre, B₃ may be optionally substituted with 1 or 2 substituents selected from alkyl, cycloalkyl, alkoxy, OH, CF₃, CN, and halo, or 1 oxo substituent;

and either:
(i) B₄ is N(R₂), B₅ is N, and B₆ is C(R₃), and B₄ is connected to B₅ via a single bond and B₅ is connected to B₆ via a double bond;
(ii) B₄ is C(R₂), B₅ is N, and B₆ is N(R₃), and B₄ is connected to B₅ via a double bond and B₅ is connected to B₆ via a single bond;
(iii) B₄ is C(R₂), B₅ is C (H), and B₆ is N(R₃), and B₄ is connected to B₅ via a double bond and B₅ is connected to B₆ via a single bond; or
R₂ is selected from H, —(CH₂)₀₋₃—C(═O)—N(R₄)(R₅), —C(═O)—O—R₆, —(CH₂)₀₋₃—NR₁₄—C(═O) R₁₅, alkyl, cycloalkyl, heterocycloalkyl, alkoxy, —OH, —CF₃, halo, aryl, heteroaryl, —CH₂—O—R₁₆ and —CN;
wherein R₄ is H, alkyl, cycloalkyl, and heterocycloalkyl, and
R₅ is selected from alkyl, —(CH₂)₀₋₃-cycloalkyl, —(CH₂)₀₋₃-heterocycloalkyl, —(CH₂)₀₋₃-polycycloalkyl, —(CH₂)₀₋₃-aryl, —(C(H)(phenyl))-alkyl, —(CH₂)₀₋₃heteroaryl^{a}, and —(C(H)(heteroaryl))-alkyl;
or wherein R₄ and R₅ together with the nitrogen atom to which they are attached form a carbon-containing 4-, 5-, 6-, or 7-membered heterocyclic ring which may be saturated or unsaturated with 1 or 2 double bonds, optionally containing one or two additional heteroatoms selected from N, O, and S, wherein the ring may be optionally substituted with 1, 2, or 3 substituents selected from oxo, alkyl, alkoxy, OH, halo and CF_{3;}
R₆ is selected from H, alkyl, —(CH₂)₀₋₃-cycloalkyl, —(CH₂)₀₋₃-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, and —(C(H)(heteroaryl))-alkyl; R₁₄ is selected from H, alkyl, cycloalkyl, and heterocycloalkyl; R₁₅ is selected from —(CH₂)₀₋₃-cycloalkyl, —(CH₂)₀₋₃-heterocycloalkyl, —(CH₂)₀₋₃-aryl, —(C(H)(phenyl))-alkyl, H, alkyl, —(CH₂)₀₋₃heteroaryl^{a}, and —(C(H)(heteroaryl))-alkyl;
R₁₆ is selected from H, alkyl, cycloalkyl, and heterocycloalkyl;
R₁ is:
(i) Formula (II),

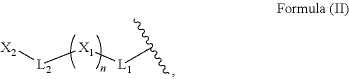

wherein:
n is 0 or 1;
L₁ is a linker selected from a single bond, —(CH₂)₁₋₃—, —(CH)(alkyl)-, and —C(alkyl)(alkyl)-; X₁ is phenyl or a 5- or 6-membered carbon-containing aromatic ring containing one, two or three ring members that are selected from N, S, and O;
wherein X₁ may be optionally substituted with 1 or 2 substituents independently selected from alkyl, cycloalkyl, alkoxy, OH, CF₃, and halo;
L₂ is a linker selected from a single bond, —(CH₂)₁₋₃—, —(CH)(alkyl)-, —C(alkyl)(alkyl)-, —C(═O)—, and —SO₂—; and

X₂ is either:
(a) —NR₂OR₂₁, wherein R₂₀ is selected from H, alkyl, and —(CH₂)₀₋₃-R_{22;}
wherein R₂₁ is selected from H, alkyl, and —(CH₂)₀₋₃-R₂₃;

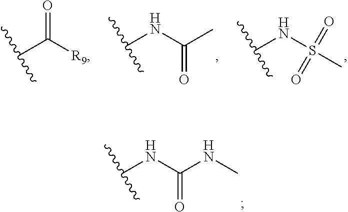

wherein R₂₂ is selected from cycloalkyl, heterocycloalkyl, aryl, heteroaryl,

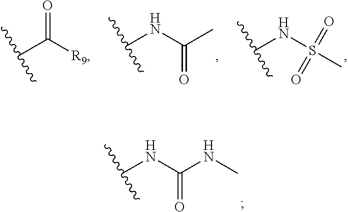

wherein R₂₃ is selected from cycloalkyl, heterocycloalkyl, aryl, heteroaryl, or
(b)₂-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected from alkyl, alkoxy, Oxo, cycloalkyl, OH, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O; or,

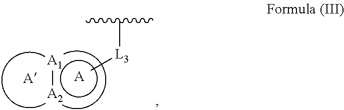

wherein:
L₃ is a linker selected from a single bond, —(CH₂)₁₋₃—, —(CH)(alkyl)-, and —C(alkyl)(alkyl)-;
A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R₇ is selected from alkyl, alkoxy, and —(CH₂)₀₋₃-R₈,

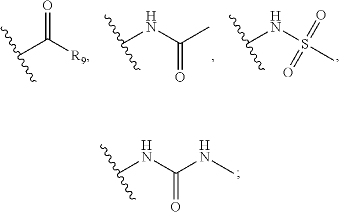

wherein R₈ is selected from cycloalkyl, heterocycloalkyl, aryl, heteroaryl, wherein R₉ is selected from H, alkyl, OH, NH₂, and alkoxy;
wherein A′ may be optionally substituted with 1 or 2 further substituents R₁₀ and R₁₁, wherein R₁₀ and R₁₁ are independently selected from alkyl, cycloalkyl, alkoxy, OH, oxo, CF₃, and halo, or wherein A′ may be optionally substituted with 1 further substituent R₂₄ selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O; and

R₃ is phenyl, or a 5- or 6-membered carbon-containing aromatic ring containing one, two or three ring members that are selected from and N, S, 0, wherein R₃ is substituted with one or two substituents independently selected from small alkyl, small alkoxy, cyclopropyl, OH, CF₃, halo, —C(═O)—NH₂, —C(═O)—N(H)(small alkyl), —C(═O)—N(small alkyl)(small alkyl), and CN; and alkyl is a linear saturated hydrocarbon having up to 10 carbon atoms (C₁-C₁₀) or a branched saturated hydrocarbon of between 3 and 10 carbon atoms (C₃-C₁₀); alkyl may optionally be substituted with 1, 2 or 3 substituents independently selected from cyclopropyl, (C₁-C₅)alkoxy, OH, —NR₁₂R₁₃, —C(═O) NR₁₂R₁₃, CN, CF₃, and halo;

small alkyl is a linear saturated hydrocarbon having up to 4 carbon atoms (C₁-C₄) or a branched saturated hydrocarbon of between 3 and 4 carbon atoms (C₃-C₄); small alkyl may optionally be substituted with 1 or 2 substituents independently selected from cyclopropyl, OH, CN, CF₃, NH₂, —C(═O) NH₂, and halo;

alkoxy is OCF₃, a linear O-linked hydrocarbon of between 1 and 6 carbon atoms (C₁-C₆), or a branched O-linked hydrocarbon of between 3 and 6 carbon atoms (C₃-C₆); alkoxy may optionally be substituted with 1 or 2 substituents independently selected from cyclopropyl, OH, CN, CF₃, and halo;

small alkoxy is OCF₃, a linear O-linked hydrocarbon of between 1 and 4 carbon atoms (C₁-C₄), or a branched O-linked hydrocarbon of between 3 and 4 carbon atoms (C₃-C₄); small alkoxy may optionally be substituted with 1 or 2 substituents independently selected from cyclopropyl, OH, CN, CF₃, and halo;

aryl is phenyl, biphenyl or naphthyl; aryl may be optionally substituted with 1, 2 or 3 substituents independently selected from alkyl, alkoxy, methylenedioxy, ethylenedioxy, OH, halo, CN, —(CH₂)₀₋₃—NR₁₂R₁₃, OCF₃ and CF_{3;}

polycycloalkyl is a bicyclic saturated hydrocarbon ring system of between 5 and 8 carbon atoms (C₅-Cs), wherein the bicyclic ring is bridged, or is adamantyl; polycycloalkyl may optionally be substituted with 1 or 2 substituents independently selected from alkyl, (C₁-C₆)alkoxy, OH, CN, CF₃, and halo;

cycloalkyl is a monocyclic saturated hydrocarbon ring of between 3 and 6 carbon atoms (C₃-C₆); cycloalkyl may optionally be substituted with 1 or 2 substituents independently selected from alkyl, (C₁-C₅)alkoxy, OH, CN, CF₃, and halo;

halo is F, Cl, Br, or I;

heteroaryl is a 5- or 6-membered carbon-containing aromatic ring containing one, two or three ring members that are selected from N, S, and O; wherein heteroaryl may be optionally substituted with 1, 2 or 3 substituents independently selected from alkyl, alkoxy, OH, halo, CN, and CF_{3;}

heteroaryl^{a} is a 5-, 6-, 9- or 10-membered mono- or bi-cyclic aromatic ring containing, where possible, 1, 2, 3 or 4 ring members independently selected from N, S, and O; wherein heteroaryl^{a} may be optionally substituted with 1, 2 or 3 substituents independently selected from alkyl, alkoxy, OH, halo, CN, and CF_{3;}

heterocycloalkyl is a non-aromatic carbon-containing mono- or bi-cyclic ring containing 3, 4, 5, 6, or 7 ring members, wherein at least one ring member is independently selected from N, S, and O; heterocycloalkyl may optionally be substituted with 1 or 2 substituents independently selected from alkyl, cyclopropyl, (C₁-C₆)alkoxy, OH, CN, CF₃, oxo, and halo; wherein when heterocycloalkyl is a bi-cyclic ring system, the bicyclic ring system is fused, bridged or spiro;

each R₁₂ and R₁₃ is independently selected from H, alkyl, cycloalkyl, and heterocycloalkyl;

and tautomers, isomers, stereoisomers (including enantiomers, diastereoisomers and racemic and scalemic mixtures thereof), deuterated analogues, and pharmaceutically acceptable salts and/or solvates thereof.

The invention in particular provides compounds of formula (I),

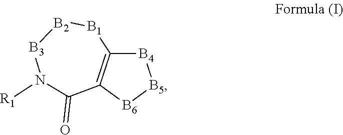

wherein:
B₁ is a carbon centre or nitrogen centre; and either:
(i) B₂ and B₃ together form a single bond between B₁ and the nitrogen atom to which R₁ is attached,
(ii) B₂ is a carbon centre or a nitrogen centre, and B₃ is a single bond between B₂ and the nitrogen atom to which R₁ is attached, wherein, when B₂ is a carbon centre or nitrogen centre, B₁ is connected to B₂ via either a single or double bond;
wherein B₁ may, where possible, be optionally substituted with 1 or 2 substituents selected from alkyl, cycloalkyl, alkoxy, OH, CF₃, CN, and halo, or 1 oxo substituent, or 2 substituents which, together with B₁ to which they are both attached, form a 3-, 4-, or 5-membered non-aromatic monocyclic carbon-containing ring, optionally wherein one or two ring members are selected from N, S, and O;

wherein, when B₂ is a carbon centre, B₂ may be optionally substituted with 1 or 2 substituents selected from alkyl, cycloalkyl, alkoxy, OH, CF₃, CN, and halo, or 1 oxo substituent;

and either:
(i) B₄ is N(R₂), B₅ is N, and B₆ is C(R₃), and B₄ is connected to B₅ via a single bond and B₅ is connected to B₆ via a double bond;
(ii) B₄ is C(R₂), B₅ is N, and B₆ is N(R₃), and B₄ is connected to B₅ via a double bond and B₅ is connected to B₆ via a single bond; or
(iii) B₄ is C(R₂), B₅ is C (H), and B₆ is N(R₃), and B₄ is connected to B₅ via a double bond and B₅ is connected to B₆ via a single bond;
R₂ is selected from H, —(CH₂)₀₋₃—C(═O)—N(R₄)(R₅), —C(═O)—O—R₆, —(CH₂)₀₋₃—NR₁₄—C(═O) R₁₅, —(CH₂)₀₋₃—NR₃₄—S(═O)₂R₃₅, alkyl, cycloalkyl, heterocycloalkyl, alkoxy, —OH, —CF₃, halo, aryl, heteroaryl, —CH₂—O—R₁₆ and —CN;
wherein R₄ is H, alkyl, cycloalkyl, and heterocycloalkyl, and
R₅ is selected from alkyl, —OH, —S(═O)₂-(small alkyl), —(CH₂)₁₋₃—(C═O)—OR₃₃, —(CH₂)₁₋₃—(C═O)—N(R₃₆)(R₃₇), —(CH₂)₀₋₃-cycloalkyl^{b}, —(CH₂)₀₋₃-heterocycloalkyl, —(CH(alkyl))-heterocycloalkyl, —(CH₂)₀₋₃-polycycloalkyl, —(CH₂)₀₋₃-aryl, —(C(H)(phenyl))-alkyl, —(CH₂)₀₋₃heteroaryl^{a}, —(C(H)(heteroaryl))-alkyl, —(C(H)(alkyl))—(C═O)—OR₃₈, —(C(H)(alkyl))—(C═O)—N(R₃₉)(R₄₀), —(C(H)(heteroaryl))—(C═O)—OR₂₈, and —(C(H)(heteroaryl))—(C═O)—N(R₄₁)(R₄₂);
or wherein R₄ and R₅ together with the nitrogen atom to which they are attached form a carbon-containing 4-, 5-, 6-, or 7-membered heterocyclic ring which may be saturated or unsaturated with 1 or 2 double bonds, optionally containing one or two additional heteroatoms selected from N, O, and S, wherein the ring may be optionally substituted with 1, 2, or 3 substituents selected from oxo, alkyl, alkoxy, OH, halo and CF₃; R₂₈, R₃₃ R₃₆, R₃₇, R₃₈, R₃₉, R₄₀, R₄₁, and R₄₂ are each independently selected from H and small alkyl; R₆ is selected from H, alkyl, —(CH₂)₀₋₃-cycloalkyl, —(CH₂)₀₋₃-heterocycloalkyl, —(CH(alkyl))-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, and —(C(H)(heteroaryl))-alkyl;
R₁₄ is selected from H, alkyl, cycloalkyl, and heterocycloalkyl;
R₁₅ is selected from H, alkyl, —(CH₂)₀₋₃-cycloalkyl, —(CH₂)₀₋₃-heterocycloalkyl, —(CH(alkyl))-heterocycloalkyl, —(CH₂)₀₋₃-aryl, —(C(H)(phenyl))-alkyl, —(CH₂)₀₋₃heteroaryl^{a}, and —(C(H)(heteroaryl))-alkyl;
R₁₆ is selected from H, alkyl, cycloalkyl, —(CH₂)₁₋₃—(C═O)—OH, heterocycloalkyl, and —(CH₂)₀₋₃heteroaryl;
R₃₄ is selected from H, alkyl, cycloalkyl, and heterocycloalkyl;
R₃₅ is selected from H, alkyl, —(CH₂)₀₋₃-cycloalkyl, —(CH₂)₀₋₃-heterocycloalkyl, —(CH(alkyl))-heterocycloalkyl, and —(C(H)(heteroaryl))-alkyl;

—(CH₂)₀₋₃-aryl, —(C(H)(phenyl))-alkyl, —(CH₂)₀₋₃heteroaryl^{a},
R₁ is:
(i) Formula (II), wherein Formula (II) is represented by Formula (IIb),

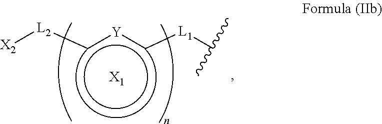

wherein:
n is 1;
L₁ is a linker selected from a single bond, —(CH₂)₁₋₃—, —(CH)(alkyl)-, and —C(alkyl)(alkyl); Y is selected from C, N, O and S;
X₁ is phenyl or a 5- or 6-membered carbon-containing aromatic ring containing one, two or three ring members that are selected from N, S, and O;
wherein X₁ may be optionally substituted with 1 or 2 substituents independently selected from alkyl, cycloalkyl, alkoxy, OH, O(cyclopropyl), CF₃, and halo;
L₂ is a linker selected from a single bond, —(CH₂)₁₋₃—, —(CH)(alkyl)-, —C(alkyl)(alkyl)-, —C(═O)—, and —SO₂—; and

X₂ is either:
(a) —NR₂OR₂₁, wherein R₂₀ is selected from H, alkyl, and —(CH₂)₀₋₃-R_{22;}
wherein R₂₁ is selected from H, alkyl, and —(CH₂)₀₋₃-R₂₃;

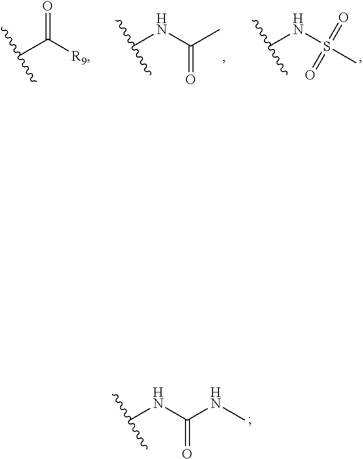

wherein R₂₂ is selected from cycloalkyl, heterocycloalkyl, aryl, heteroaryl,

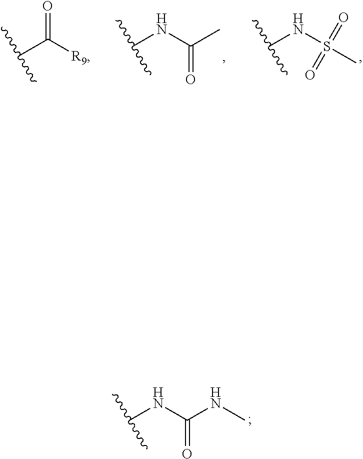

wherein R₂₃ is selected from cycloalkyl, heterocycloalkyl, aryl, heteroaryl, or
(b)₂-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O, and X₂ may be further optionally substituted with 1 or 2 substituents R₂₉ and R₃₀, wherein R₂₉ and R₃₀ are independently selected from alkyl, cycloalkyl, alkoxy, OH, oxo, CF₃, and halo; or,
(ii) Formula (III) comprising an aromatic ring A fused to a non-aromatic ring A′,

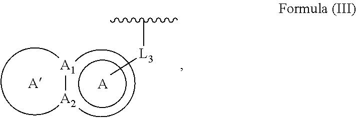

wherein:
L₃ is a linker selected from a single bond, —(CH₂)₁₋₃—, —(CH)(alkyl)-, and —C(alkyl)(alkyl)-;
A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, O(cyclopropyl), CF₃, and halo;

A′ is a non-aromatic carbon-containing monocyclic ring containing 5, 6, or 7, ring members, wherein at least one ring member is independently selected from N, S, and O; wherein A′ may be optionally substituted with one R₇, wherein R₇ is selected from alkyl, alkoxy, and —(CH₂)₀₋₃-R₈,

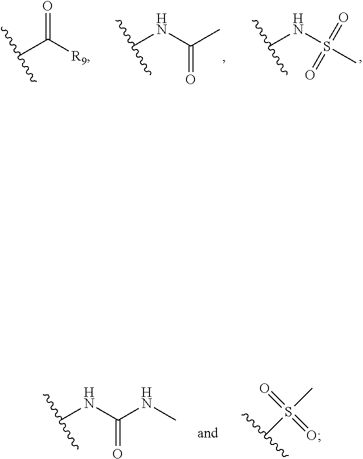

wherein R₈ is selected from cycloalkyl, heterocycloalkyl, aryl, heteroaryl, wherein R₉ is selected from H, alkyl, OH, N(R₃₁)(R₃₂), and alkoxy;
wherein R₃₁ is H or small alkyl;
wherein R₃₂ is selected from H, small alkyl, and —(CH₂)₀₋₃heteroaryl;
wherein A′ may be optionally substituted with 1, 2, or 3 further substituents R₁₀, R₁₁, and
R₁₇, wherein R₁₀, R₁₁, and R₁₇, are independently selected from alkyl, cycloalkyl, alkoxy, OH, oxo, CF₃, and halo, or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O, or wherein A′ may be optionally substituted with 1 or 2 further substituents R₄₃ and R₄₄ independently selected from alkyl, cycloalkyl, alkoxy, OH, oxo, CF₃, and halo, and two different carbon ring members within A′ are connected by a bridging —CH₂-group; and

R₃ is phenyl, or a 5- or 6-membered carbon-containing aromatic ring containing one, two or three ring members that are selected from N, S, and O, wherein R₃ is substituted with one, two, or three substituents independently selected from small alkyl^{b}, small alkoxy, cyclopropyl, OH, NH₂, CF₃, halo, —C(═O)—NH₂,

—C(═O)—N(H)(small alkyl), —C(═O)—N(small alkyl)(small alkyl), and CN; wherein R₃ is not

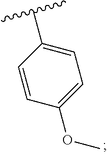 [SMILES: *c1ccc(OC)cc1]

and
alkyl is a linear saturated hydrocarbon having up to 10 carbon atoms (C₁-C₁₀) or a branched saturated hydrocarbon of between 3 and 10 carbon atoms (C₃-C₁₀); alkyl may optionally be substituted with 1, 2 or 3 substituents independently selected from cyclopropyl, (C₁-C₅)alkoxy, OH, —NR₁₂R₁₃, —C(═O) NR₁₂R₁₃, CN, CF₃, and halo;

small alkyl is a linear saturated hydrocarbon having up to 4 carbon atoms (C₁-C₄) or a branched saturated hydrocarbon of between 3 and 4 carbon atoms (C₃-C₄); small alkyl may optionally be substituted with 1 or 2 substituents independently selected from cyclopropyl, OH, CN, CF₃, NH₂, —C(═O) NH₂, and halo;
small alkyl^{b} is a linear saturated hydrocarbon having up to 4 carbon atoms (C₁-C₄) or a branched saturated hydrocarbon of between 3 and 4 carbon atoms (C₃-C₄); small alkyl may optionally be substituted with 1 or 2 substituents independently selected from cyclopropyl, OH, OMe, OEt, OiPr, O″Pr, O(cyclopropyl), OCF₃, OCHF₂, CN, CF₃, NH₂, —C(═O) NH₂, and halo;
alkoxy is OCF₃, O(cyclopropyl), a linear O-linked hydrocarbon of between 1 and 6 carbon atoms (C₁-C₆), or a branched O-linked hydrocarbon of between 3 and 6 carbon atoms (C₃-C₆); alkoxy may optionally be substituted with 1 or 2 substituents independently selected from cyclopropyl, OH, CN, CF₃, and halo;
small alkoxy is OCF₃, O(cyclopropyl), a linear O-linked hydrocarbon of between 1 and 4 carbon atoms (C₁-C₄), or a branched O-linked hydrocarbon of between 3 and 4 carbon atoms (C₃-C₄); small alkoxy may optionally be substituted with 1 or 2 substituents independently selected from cyclopropyl, OH, CN, CF₃, and halo;
aryl is phenyl, biphenyl or naphthyl; aryl may be optionally substituted with 1, 2 or 3 substituents independently selected from alkyl, alkoxy, methylenedioxy, ethylenedioxy, OH, halo, CN, —(CH₂)₀₋₃—NR₁₂R₁₃, OCF₃ and CF_{3;}
polycycloalkyl is a bicyclic saturated hydrocarbon ring system of between 5 and 8 carbon atoms (C₅-Cs), wherein the bicyclic ring is bridged, or is adamantyl; polycycloalkyl may optionally be substituted with 1 or 2 substituents independently selected from alkyl, (C₁-C₆)alkoxy, OH, CN, CF₃, and halo;
cycloalkyl is a monocyclic saturated hydrocarbon ring of between 3 and 6 carbon atoms (C₃-C₆); cycloalkyl may optionally be substituted with 1 or 2 substituents independently selected from alkyl, (C₁-C₆)alkoxy, OH, CN, CF₃, and halo cycloalkylª is a monocyclic saturated hydrocarbon ring of between 3 and 6 carbon atoms (C₃-C₆); cycloalkyl may optionally be substituted with 1 or 2 substituents independently selected from alkyl, (C₁-C₆)alkoxy, OH, CN, CF₃, halo, and —(C═O)—OH;
cycloalkyl^{b} is a monocyclic saturated hydrocarbon ring of between 3 and 6 carbon atoms (C₃-C₆);
cycloalkyl may optionally be substituted with 1 or 2 substituents independently selected from alkyl, (C₁-C₆)alkoxy, OH, NH₂, NH (small alkyl), CN, CF₃, halo, and —(C═O)—OH;
halo is F, Cl, Br, or I;
heteroaryl is a 5- or 6-membered carbon-containing aromatic ring containing one, two or three ring members that are selected from N, S, and O; wherein heteroaryl may be optionally substituted with 1, 2 or 3 substituents independently selected from alkyl, alkoxy, cyclopropyl, OH, halo, CN, and CF_{3;}
heteroaryl^{a} is a 5-, 6-, 9- or 10-membered mono- or bi-cyclic aromatic ring containing, where possible, 1, 2, 3 or 4 ring members independently selected from N, S, and O; wherein heteroaryl^{a} may be optionally substituted with 1, 2 or 3 substituents independently selected from alkyl, alkoxy, cyclopropyl, OH, halo, CN, and CF_{3;}
heterocycloalkyl is a non-aromatic carbon-containing mono- or bi-cyclic ring containing 3, 4, 5, 6, or 7 ring members, wherein at least one ring member is independently selected from N, S, and O; heterocycloalkyl may optionally be substituted with 1 or 2 substituents independently selected from alkyl, cyclopropyl, (C₁-C₅)alkoxy, OH, CN, CF₃, oxo, and halo; wherein when heterocycloalkyl is a bi-cyclic ring system, the bicyclic ring system is fused, bridged or spiro;
each R₁₂ and R₁₃ is independently selected from H, alkyl, cycloalkyl, and heterocycloalkyl;
and tautomers, isomers, stereoisomers (including enantiomers, diastereoisomers and racemic and scalemic mixtures thereof), deuterated analogues, and pharmaceutically acceptable salts and/or solvates thereof.

The invention in particular also provides compounds of formula (I),

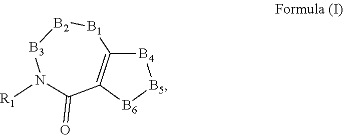

wherein:
B₁ is a carbon centre or nitrogen centre; and either:
(i) B₂ and B₃ together form a single bond between B₁ and the nitrogen atom to which R₁ is attached,
(ii) B₂ is a carbon centre or a nitrogen centre, and B₃ is a single bond between B₂ and the nitrogen atom to which R₁ is attached,
(iii) B₂ is a carbon centre and B₃ is a nitrogen centre, or
(iv) B₂ and B₃ are both carbon centres, wherein, when B₂ is a carbon centre or nitrogen centre, B₁ is connected to B₂ via either a single or double bond;

wherein, when both B₂ and B₃ are present, B₂ and B₃ are connected via either a single or double bond;
wherein B₁ may, where possible, be optionally substituted with 1 or 2 substituents selected from alkyl, cycloalkyl, alkoxy, OH, CF₃, CN, and halo, or 1 oxo substituent, or 2 substituents which, together with B₁ to which they are both attached, form a 3-, 4-, or 5-membered non-aromatic monocyclic carbon-containing ring, optionally wherein one or two ring members are selected from N, S, and O;

wherein, when B₂ is a carbon centre, B₂ may be optionally substituted with 1 or 2 substituents selected from alkyl, cycloalkyl, alkoxy, OH, CF₃, CN, and halo, or 1 oxo substituent; and

wherein, when B₃ is a carbon centre, B₃ may be optionally substituted with 1 or 2 substituents selected from alkyl, cycloalkyl, alkoxy, OH, CF₃, CN, and halo, or 1 oxo substituent;

and either:
(i) B₄ is N(R₂), B₅ is N, and B₆ is C(R₃), and B₄ is connected to B₅ via a single bond and B₅ is connected to B₆ via a double bond;
(ii) B₄ is C(R₂), B₅ is N, and B₆ is N(R₃), and B₄ is connected to B₅ via a double bond and B₅ is connected to B₆ via a single bond; or
(iii) B₄ is C(R₂), B₅ is C (H), and B₆ is N(R₃), and B₄ is connected to B₅ via a double bond and B₅ is connected to B₆ via a single bond;
R₂ is selected from H, —(CH₂)₀₋₃—C(═O)—N(R₄)(R₅), —C(═O)—O—R₆, —(CH₂)₀₋₃—NR₁₄—C(═O) R₁₅, —(CH₂)₀₋₃—NR₃₄—S(═O)₂R₃₅, alkyl, cycloalkyl, heterocycloalkyl, alkoxy, —OH, —CF₃, halo, aryl, heteroaryl, —CH₂—O—R₁₆ and —CN;
wherein R₄ is H, alkyl, cycloalkyl, and heterocycloalkyl, and

R₅ is selected from alkyl, —(CH₂)₀₋₃-cycloalkyl, —(CH₂)₀₋₃-heterocycloalkyl, —(CH(alkyl)-heterocycloalkyl, —(CH₂)₀₋₃-polycycloalkyl, —(CH₂)₀₋₃-aryl, —(C(H)(phenyl))-alkyl, —(CH₂)₀₋₃heteroaryl^{a}, and —(C(H)(heteroaryl))-alkyl;
or wherein R₄ and R₅ together with the nitrogen atom to which they are attached form a carbon-containing 4-, 5-, 6-, or 7-membered heterocyclic ring which may be saturated or unsaturated with 1 or 2 double bonds, optionally containing one or two additional heteroatoms selected from N, O, and S, wherein the ring may be optionally substituted with 1, 2, or 3 substituents selected from oxo, alkyl, alkoxy, OH, halo and CF_{3;}
R₆ is selected from H, alkyl, —(CH₂)₀₋₃-cycloalkyl, —(CH₂)₀₋₃-heterocycloalkyl, —(CH(alkyl)-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, and —(C(H)(heteroaryl))-alkyl;
R₁₄ is selected from H, alkyl, cycloalkyl, and heterocycloalkyl;
R₁₅ is selected from H, alkyl, —(CH₂)₀₋₃-cycloalkyl, —(CH₂)₀₋₃-heterocycloalkyl, —(CH(alkyl))-heterocycloalkyl, —(CH₂)₀₋₃-aryl, —(C(H)(phenyl))-alkyl, —(CH₂)₀₋₃heteroaryl^{a}, and —(C(H)(heteroaryl))-alkyl; R₁₆ is selected from H, alkyl, cycloalkyl, and heterocycloalkyl; R₃₄ is selected from H, alkyl, cycloalkyl, and heterocycloalkyl; R₃₅ is selected from H, alkyl, —(CH₂)₀₋₃-cycloalkyl, —(CH₂)₀₋₃-heterocycloalkyl, —(CH(alkyl)-heterocycloalkyl, —(CH₂)₀₋₃-aryl, —(C(H)(phenyl))-alkyl, —(CH₂)₀₋₃heteroaryl^{a}, and —(C(H)(heteroaryl))-alkyl;
R₁ is:

i) Formula (II), wherein Formula (II) is represented by Formula (IIb),

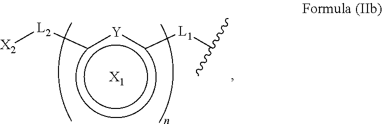

wherein:
n is 1;
L₁ is a linker selected from a single bond, —(CH₂)₁₋₃—, —(CH)(alkyl)-, and —C(alkyl)(alkyl)-; Y is selected from C, N, O and S;
X₁ is phenyl or a 5- or 6-membered carbon-containing aromatic ring containing one, two or three ring members that are selected from N, S, and O;
wherein X₁ may be optionally substituted with 1 or 2 substituents independently selected from alkyl, cycloalkyl, alkoxy, OH, CF₃, and halo;
L₂ is a linker selected from a single bond, —(CH₂)₁₋₃—, —(CH)(alkyl)-, —C(alkyl)(alkyl)-, —C(═O)—, and —SO₂—; and

X₂ is either:
(a) —NR₂OR₂₁, wherein R₂₀ is selected from H, alkyl, and —(CH₂)₀₋₃-R_{22;}
wherein R₂₁ is selected from H, alkyl, and —(CH₂)₀₋₃-R₂₃;

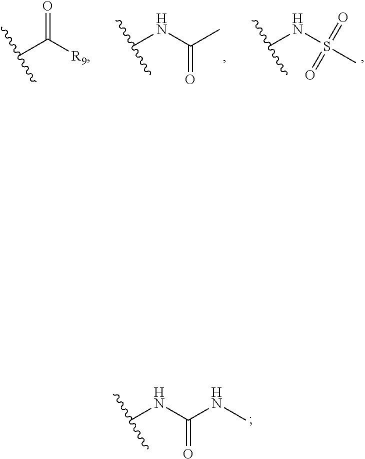

wherein R₂₂ is selected from cycloalkyl, heterocycloalkyl, aryl, heteroaryl,
wherein R₂₃ is selected from cycloalkyl, heterocycloalkyl, aryl, heteroaryl,

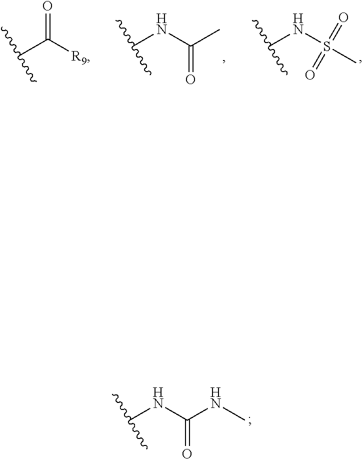

or
(b)₂-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O; or, (ii) Formula (III) comprising an aromatic ring A fused to a non-aromatic ring A′,

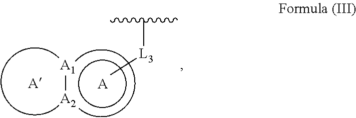

wherein:
L₃ is a linker selected from a single bond, —(CH₂)₁₋₃—, —(CH)(alkyl)-, and —C(alkyl)(alkyl)-;
A₁ and Az are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, CF₃, and halo;

A′ is a non-aromatic carbon-containing monocyclic ring containing 5, 6, or 7, ring members, wherein at least one ring member is independently selected from N, S, and O; wherein A′ may be optionally substituted with one R₇, wherein R₇ is selected from alkyl, alkoxy, and —(CH₂)₀₋₃-R₈,
wherein R₈ is selected from cycloalkyl, heterocycloalkyl, aryl, heteroaryl,

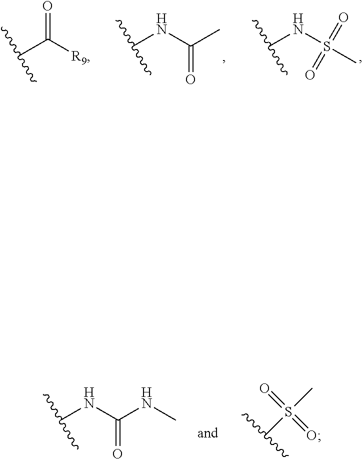

wherein R₉ is selected from H, alkyl, OH, NH₂, and alkoxy;
wherein A′ may be optionally substituted with 1, 2, or 3 further substituents R₁₀, R₁₁, and R₁₇, wherein R₁₀, R₁₁, and R₁₇, are independently selected from alkyl, cycloalkyl, alkoxy, OH, oxo, CF₃, and halo, or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O; and

R₃ is phenyl, or a 5- or 6-membered carbon-containing aromatic ring containing one, two or three ring members that are selected from N, S, and O, wherein R₃ is substituted with one or two substituents independently selected from small alkyl, small alkoxy, cyclopropyl, OH, CF₃, halo, —C(═O)—NH₂, —C(═O)—N(H)(small alkyl),

—C(═O)—N(small alkyl)(small alkyl), and CN; wherein R₃ is not

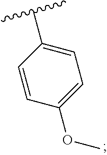 [SMILES: *c1ccc(OC)cc1]

and alkyl is a linear saturated hydrocarbon having up to 10 carbon atoms (C₁-C₁₀) or a branched saturated hydrocarbon of between 3 and 10 carbon atoms (C₃-C₁₀); alkyl may optionally be substituted with 1, 2 or 3 substituents independently selected from cyclopropyl, (C₁-C₆)alkoxy, OH, —NR₁₂R₁₃, —C(═O) NR₁₂R₁₃, CN, CF₃, and halo;

small alkyl is a linear saturated hydrocarbon having up to 4 carbon atoms (C₁-C₄) or a branched saturated hydrocarbon of between 3 and 4 carbon atoms (C₃-C₄); small alkyl may optionally be substituted with 1 or 2 substituents independently selected from cyclopropyl, OH, CN, CF₃, NH₂, —C(═O) NH₂, and halo;

alkoxy is OCF₃, a linear O-linked hydrocarbon of between 1 and 6 carbon atoms (C₁-C₆), or a branched O-linked hydrocarbon of between 3 and 6 carbon atoms (C₃-C₆); alkoxy may optionally be substituted with 1 or 2 substituents independently selected from cyclopropyl, OH, CN, CF₃, and halo;

small alkoxy is OCF₃, a linear O-linked hydrocarbon of between 1 and 4 carbon atoms (C₁-C₄), or a branched O-linked hydrocarbon of between 3 and 4 carbon atoms (C₃-C₄); small alkoxy may optionally be substituted with 1 or 2 substituents independently selected from cyclopropyl, OH, CN, CF₃, and halo;

aryl is phenyl, biphenyl or naphthyl; aryl may be optionally substituted with 1, 2 or 3 substituents independently selected from alkyl, alkoxy, methylenedioxy, ethylenedioxy, OH, halo, CN, —(CH₂)₀₋₃—NR₁₂R₁₃, OCF₃ and CF_{3;}

polycycloalkyl is a bicyclic saturated hydrocarbon ring system of between 5 and 8 carbon atoms (C₅-Cs), wherein the bicyclic ring is bridged, or is adamantyl; polycycloalkyl may optionally be substituted with 1 or 2 substituents independently selected from alkyl, (C₁-C₆)alkoxy, OH, CN, CF₃, and halo;

cycloalkyl is a monocyclic saturated hydrocarbon ring of between 3 and 6 carbon atoms (C₃-C₆);

cycloalkyl may optionally be substituted with 1 or 2 substituents independently selected from alkyl, (C₁-C₆)alkoxy, OH, CN, CF₃, and halo;

halo is F, Cl, Br, or I;

heteroaryl is a 5- or 6-membered carbon-containing aromatic ring containing one, two or three ring members that are selected from N, S, and O; wherein heteroaryl may be optionally substituted with 1, 2 or 3 substituents independently selected from alkyl, alkoxy, cyclopropyl, OH, halo, CN, and CF_{3;}

heteroaryl^{a} is a 5-, 6-, 9- or 10-membered mono- or bi-cyclic aromatic ring containing, where possible, 1, 2, 3 or 4 ring members independently selected from N, S, and O; wherein heteroaryl^{a} may be optionally substituted with 1, 2 or 3 substituents independently selected from alkyl, alkoxy, cyclopropyl, OH, halo, CN, and CF_{3;}

heterocycloalkyl is a non-aromatic carbon-containing mono- or bi-cyclic ring containing 3, 4, 5, 6, or 7 ring members, wherein at least one ring member is independently selected from N, S, and O;

heterocycloalkyl may optionally be substituted with 1 or 2 substituents independently selected from alkyl, cyclopropyl, (C₁-C₅)alkoxy, OH, CN, CF₃, oxo, and halo; wherein when heterocycloalkyl is a bi-cyclic ring system, the bicyclic ring system is fused, bridged or spiro;

each R₁₂ and R₁₃ is independently selected from H, alkyl, cycloalkyl, and heterocycloalkyl;

and tautomers, isomers, stereoisomers (including enantiomers, diastereoisomers and racemic and scalemic mixtures thereof), deuterated analogues, and pharmaceutically acceptable salts and/or solvates thereof.

The invention in particular also provides compounds of formula (I),

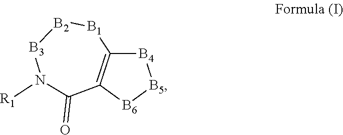

wherein:
B₁ is a carbon centre or nitrogen centre; and either:
(i) B₂ and B₃ together form a single bond between B₁ and the nitrogen atom to which R₁ is attached,
(ii) B₂ is a carbon centre or a nitrogen centre, and B₃ is a single bond between B₂ and the nitrogen atom to which R₁ is attached,
(iii) B₂ is a carbon centre and B₃ is a nitrogen centre, or
(iv) B₂ and B₃ are both carbon centres, wherein, when B₂ is a carbon centre or nitrogen centre, B₁ is connected to B₂ via either a single or double bond;

wherein, when both B₂ and B₃ are present, B₂ and B₃ are connected via either a single or double bond;
wherein B₁ may, where possible, be optionally substituted with 1 or 2 substituents selected from alkyl, cycloalkyl, alkoxy, OH, CF₃, CN, and halo, or 1 oxo substituent, or 2 substituents which, together with B₁ to which they are both attached, form a 3-, 4-, or 5-membered non-aromatic monocyclic carbon-containing ring, optionally wherein one or two ring members are selected from N, S, and O;

wherein, when B₂ is a carbon centre, B₂ may be optionally substituted with 1 or 2 substituents selected from alkyl, cycloalkyl, alkoxy, OH, CF₃, CN, and halo, or 1 oxo substituent; and

wherein, when B₃ is a carbon centre, B₃ may be optionally substituted with 1 or 2 substituents selected from alkyl, cycloalkyl, alkoxy, OH, CF₃, CN, and halo, or 1 oxo substituent;

and either:
(i) B₄ is N(R₂), B₅ is N, and B₆ is C(R₃), and B₄ is connected to B₅ via a single bond and B₅ is connected to B₆ via a double bond;
(ii) B₄ is C(R₂), B₅ is N, and B₆ is N(R₃), and B₄ is connected to B₅ via a double bond and B₅ is connected to B₆ via a single bond; or
(iii) B₄ is C(R₂), B₅ is C (H), and B₆ is N(R₃), and B₄ is connected to B₅ via a double bond and B₅ is connected to B₆ via a single bond;
R₂ is selected from H, —(CH₂)₀₋₃—C(═O)—N(R₄)(R₅), —C(═O)—O—R₆, —(CH₂)₀₋₃—NR₁₄—C(═O) R₁₅, —(CH₂)₀₋₃—NR₃₄—S(═O)₂R₃₅, alkyl, cycloalkyl, heterocycloalkyl, alkoxy, —OH, —CF₃, halo, aryl, heteroaryl, —CH₂—O—R₁₆ and —CN;
wherein R₄ is H, alkyl, cycloalkyl, and heterocycloalkyl, and R₅ is selected from alkyl, —OH, —S(═O)₂-(small alkyl), —(CH₂)₁₋₃—(C═O)—OR₃₃, —(CH₂)₁₋₃—(C═O)—N(R₃₆)(R₃₇), —(CH₂)₀₋₃-cycloalkyl^{a}, —(CH₂)₀₋₃-heterocycloalkyl, —(CH(alkyl))-heterocycloalkyl, —(CH₂)₀₋₃-polycycloalkyl, —(CH₂)₀₋₃-aryl, —(C(H)(phenyl))-alkyl, —(CH₂)₀₋₃heteroaryl^{a}, —(C(H)(heteroaryl))-alkyl, —(C(H)(alkyl))—(C═O)—OR₃₈, —(C(H)(alkyl))—(C═O)—N(R₃₉)(R₄₀), —(C(H)(heteroaryl))—(C═O)—OR₂₈, and —(C(H)(heteroaryl))—(C═O)—N(R₄₁)(R₄₂);
or wherein R₄ and R₅ together with the nitrogen atom to which they are attached form a carbon-containing 4-, 5-, 6-, or 7-membered heterocyclic ring which may be saturated or unsaturated with 1 or 2 double bonds, optionally containing one or two additional heteroatoms selected from N, O, and S, wherein the ring may be optionally substituted with 1, 2, or 3 substituents selected from oxo, alkyl, alkoxy, OH, halo and CF_{3;}
R₂₈, R₃₃ R₃₆, R₃₇, R₃₈, R₃₉, R₄₀, R₄₁, and R₄₂ are each independently selected from H and small alkyl; R₆ is selected from H, alkyl, —(CH₂)₀₋₃-cycloalkyl, —(CH₂)₀₋₃-heterocycloalkyl, —(CH(alkyl))-heterocycloalkyl, —(CH₂)₀₋₃-polycycloalkyl, —(CH₂)₀₋₃-aryl, I, —(C(H)(phenyl))-alkyl, —(CH₂)₀₋₃heteroaryl, and —(C(H)(heteroaryl))-alkyl;
R₁₄ is selected from H, alkyl, cycloalkyl, and heterocycloalkyl;
R₁₅ is selected from H, alkyl, —(CH₂)₀₋₃-cycloalkyl, —(CH₂)₀₋₃-heterocycloalkyl, —(CH(alkyl))-heterocycloalkyl, —(CH₂)₀₋₃-aryl, —(C(H)(phenyl))-alkyl, —(CH₂)₀₋₃heteroaryl^{a}, and —(C(H)(heteroaryl))-alkyl;
R 16 is selected from H, alkyl, cycloalkyl, —(CH₂)₁₋₃—(C═O)—OH, heterocycloalkyl and —(CH₂)₀₋₃heteroaryl; R₃₄ is selected from H, alkyl, cycloalkyl, and heterocycloalkyl;
R₃₅ is selected from H, alkyl, —(CH₂)₀₋₃-cycloalkyl, —(CH₂)₀₋₃-heterocycloalkyl, —(CH(alkyl))-heterocycloalkyl, —(CH₂)₀₋₃-aryl, —(C(H)(phenyl))-alkyl, —(CH₂)₀₋₃heteroaryl^{a}, and —(C(H)(heteroaryl))-alkyl;
R₁ is:
(i) Formula (II), wherein Formula (II) is represented by Formula (IIb),

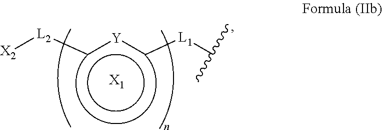

wherein:
n is 1;
L₁ is a linker selected from a single bond, —(CH₂)₁₋₃—, —(CH)(alkyl)-, and —C(alkyl)(alkyl);
Y is selected from C, N, O and S;
X₁ is phenyl or a 5- or 6-membered carbon-containing aromatic ring containing one, two or three ring members that are selected from N, S, and O;
wherein X₁ may be optionally substituted with 1 or 2 substituents independently selected from alkyl, cycloalkyl, alkoxy, OH, CF₃, and halo;
L₂ is a linker selected from a single bond, —(CH₂)₁₋₃—, —(CH)(alkyl)-, —C(alkyl)(alkyl)-, —C(═O)—, and —SO₂—; and

X₂ is either:
(a) —NR₂OR₂₁, wherein R₂₀ is selected from H, alkyl, and —(CH₂)₀₋₃-R_{22;}
wherein R₂₁ is selected from H, alkyl, and —(CH₂)₀₋₃-R₂₃;

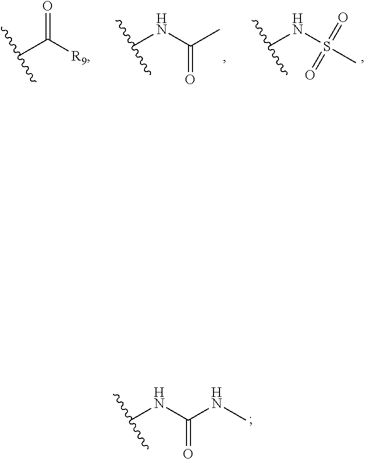

wherein R₂₂ is selected from cycloalkyl, heterocycloalkyl, aryl, heteroaryl,

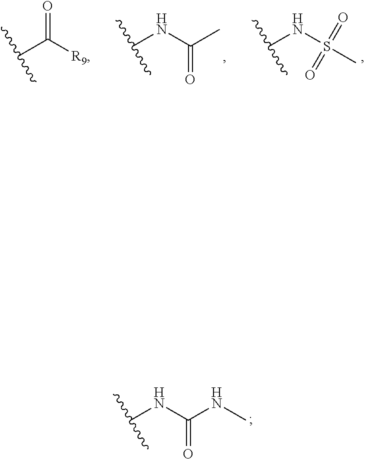

wherein R₂₃ is selected from cycloalkyl, heterocycloalkyl, aryl, heteroaryl, or
(b)₂-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O, and X₂ may be further optionally substituted with 1 or 2 substituents R₂₉ and R₃₀, wherein R₂₉ and R₃₀ are independently selected from alkyl, cycloalkyl, alkoxy, OH, oxo, CF₃, and halo; or,
(ii) Formula (III) comprising an aromatic ring A fused to a non-aromatic ring A′,

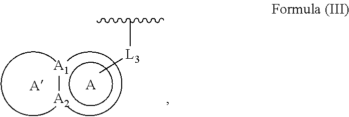

wherein:
L₃ is a linker selected from a single bond, —(CH₂)₁₋₃—, —(CH)(alkyl)-, and —C(alkyl)(alkyl);
A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, CF₃, and halo;

A′ is a non-aromatic carbon-containing monocyclic ring containing 5, 6, or 7, ring members, wherein at least one ring member is independently selected from N, S, and O; wherein A′ may be optionally substituted with one R₇, wherein R₇ is selected from alkyl, alkoxy, and —(CH₂)₀₋₃-R₈,

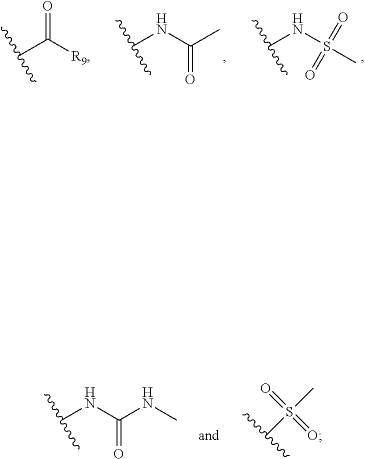

wherein R₈ is selected from cycloalkyl, heterocycloalkyl, aryl, heteroaryl, wherein R₉ is selected from H, alkyl, OH, N(R₃₁)(R₃₂), and alkoxy;
wherein R₃₁ is H or small alkyl;
wherein R₃₂ is selected from H, small alkyl, and —(CH₂)₀₋₃heteroaryl;
wherein A′ may be optionally substituted with 1, 2, or 3 further substituents R₁₀, R₁₁, and R₁₇, wherein R₁₀, R₁₁, and R₁₇, are independently selected from alkyl, cycloalkyl, alkoxy, OH, oxo, CF₃, and halo, or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O; and

R₃ is phenyl, or a 5- or 6-membered carbon-containing aromatic ring containing one, two or three ring members that are selected from N, S, and O, wherein R₃ is substituted with one or two substituents independently selected from small alkyl, small alkoxy, cyclopropyl, OH, CF₃, halo, —C(═O)—NH₂, —C(═O)—N(H)(small alkyl),

—C(═O)—N(small alkyl)(small alkyl), and CN; wherein R₃ is not

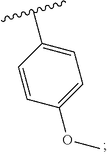 [SMILES: *c1ccc(OC)cc1]

and
alkyl is a linear saturated hydrocarbon having up to 10 carbon atoms (C₁-C₁₀) or a branched saturated hydrocarbon of between 3 and 10 carbon atoms (C₃-C₁₀); alkyl may optionally be substituted with 1, 2 or 3 substituents independently selected from cyclopropyl, (C₁-C₅)alkoxy, OH, —NR₁₂R₁₃, —C(═O) NR₁₂R₁₃, CN, CF₃, and halo;
small alkyl is a linear saturated hydrocarbon having up to 4 carbon atoms (C₁-C₄) or a branched saturated hydrocarbon of between 3 and 4 carbon atoms (C₃-C₄); small alkyl may optionally be substituted with 1 or 2 substituents independently selected from cyclopropyl, OH, CN, CF₃, NH₂, —C(═O) NH₂, and halo;
alkoxy is OCF₃, a linear O-linked hydrocarbon of between 1 and 6 carbon atoms (C₁-C₆), or a branched O-linked hydrocarbon of between 3 and 6 carbon atoms (C₃-C₆); alkoxy may optionally be substituted with 1 or 2 substituents independently selected from cyclopropyl, OH, CN, CF₃, and ha1lo;

small alkoxy is OCF₃, a linear O-linked hydrocarbon of between 1 and 4 carbon atoms (C₁-C₄), or a branched O-linked hydrocarbon of between 3 and 4 carbon atoms (C₃-C₄); small alkoxy may optionally be substituted with 1 or 2 substituents independently selected from cyclopropyl, OH, CN, CF₃, and halo;
aryl is phenyl, biphenyl or naphthyl; aryl may be optionally substituted with 1, 2 or 3 substituents independently selected from alkyl, alkoxy, methylenedioxy, ethylenedioxy, OH, halo, CN, —(CH₂)₀₋₃—NR₁₂R₁₃, OCF₃ and CF_{3;}
polycycloalkyl is a bicyclic saturated hydrocarbon ring system of between 5 and 8 carbon atoms (C₅-Cs), wherein the bicyclic ring is bridged, or is adamantyl; polycycloalkyl may optionally be substituted with 1 or 2 substituents independently selected from alkyl, (C₁-C₅)alkoxy, OH, CN, CF₃, and halo;
cycloalkyl is a monocyclic saturated hydrocarbon ring of between 3 and 6 carbon atoms (C₃-C₆); cycloalkyl may optionally be substituted with 1 or 2 substituents independently selected from alkyl, (C₁-C₆)alkoxy, OH, CN, CF₃, and halo
cycloalkyl^{a} is a monocyclic saturated hydrocarbon ring of between 3 and 6 carbon atoms (C₃-C₆); cycloalkyl may optionally be substituted with 1 or 2 substituents independently selected from alkyl, (C₁-C₆)alkoxy, OH, CN, CF₃, halo, and —(C═O)—OH;
halo is F, Cl, Br, or I;
heteroaryl is a 5- or 6-membered carbon-containing aromatic ring containing one, two or three ring members that are selected from N, S, and O; wherein heteroaryl may be optionally substituted with 1, 2 or 3 substituents independently selected from alkyl, alkoxy, cyclopropyl, OH, halo, CN, and CF_{3;}
heteroaryl^{a} is a 5-, 6-, 9- or 10-membered mono- or bi-cyclic aromatic ring containing, where possible, 1, 2, 3 or 4 ring members independently selected from N, S, and O; wherein heteroaryl^{a} may be optionally substituted with 1, 2 or 3 substituents independently selected from alkyl, alkoxy, cyclopropyl, OH, halo, CN, and CF_{3;}
heterocycloalkyl is a non-aromatic carbon-containing mono- or bi-cyclic ring containing 3, 4, 5, 6, or 7 ring members, wherein at least one ring member is independently selected from N, S, and O;

heterocycloalkyl may optionally be substituted with 1 or 2 substituents independently selected from alkyl, cyclopropyl, (C₁-C₆)alkoxy, OH, CN, CF₃, oxo, and halo; wherein when heterocycloalkyl is a bi-cyclic ring system, the bicyclic ring system is fused, bridged or spiro;
each R₁₂ and R₁₃ is independently selected from H, alkyl, cycloalkyl, and heterocycloalkyl;
and tautomers, isomers, stereoisomers (including enantiomers, diastereoisomers and racemic and scalemic mixtures thereof), deuterated analogues, and pharmaceutically acceptable salts and/or solvates thereof.

The compounds of formula (I) have been developed to be inhibitors of FXIIa, which as noted above, has a unique and specific binding site and there is a need for small molecule FXIIa inhibitors. Furthermore, compounds of the invention have been carefully developed to show selectivity for FXIIa inhibition over other inhibition of other serine proteases, thus reducing the risk of off-target effects and cytotoxicity. In particular, it was one aim of the present invention to provide compounds that show selectivity for FXIIa inhibition over FXa inhibition, such as in the selectivity shown in Table 8. It is also an aim of the present invention to provide compounds that possess characteristics that can be considered suitable for oral delivery e.g. a suitable oral pharmacokinetic profile such as the data shown in Table 10 below. The compounds of the invention can also avoid including groups associated with covalent binding properties e.g. groups with acylating reactivity such as acylated aminotriazoles, and thus can provide compounds that are reversible inhibitors, to further reduce the risk of off-target effects and cytotoxicity.

The present invention also provides a prodrug of a compound as herein defined, or a pharmaceutically acceptable salt and/or solvate thereof.

The present invention also provides an N-oxide of a compound as herein defined, or pharmaceutically acceptable salt and/or solvate thereof.

It will be understood that “methylenedioxy” refers to —O—CH₂—O-only, wherein “methylenedioxy” cannot be substituted.

It will be understood that “ethylenedioxy” refers to —O—CH₂—CH₂—O-only, wherein “ethylenedioxy” cannot be substituted.

It will be understood that “aryl substituted with methylenedioxy” refers to aryl as defined above, wherein

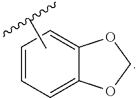

aryl is substituted with —O—CH₂—O—. For example,
It will be understood that “aryl substituted with ethylenedioxy” refers to aryl as defined above, wherein

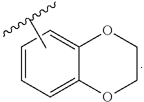

aryl is substituted with —O—CH₂—CH₂—O—. For example,
It will be understood that when B₂ and B₃ together form a single bond between B₁ and the nitrogen atom

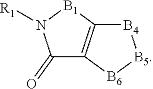

to which R₁ is attached, Formula (I) may be represented by.

It will be understood that B₂ is a carbon centre or a nitrogen centre, and B₃ is a single bond between B₂

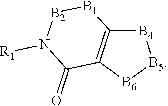

and the nitrogen atom to which R₁ is attached, Formula (I) may be represented by

It will be understood that “carbon centre” and “nitrogen centre” refer to respectively to carbon and nitrogen atom ring members, which may be substituted as described above. Where there are free valencies, it will be understood that these free valencies will be attach to a hydrogen to generate a stable compound.

It will be understood that Formula (IIb):

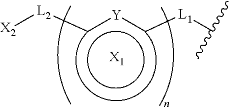

fixes a single ring atom Y on X₁ as being directly between the positions to which L₁ and L₂ attach. Alternatively, the L₁ and L₂ groups can be seen as being “1,3-substituted” on X₁, where the 2 position on the ring is where ring atom Y is found.

It will be understood that the connecting lines in formulae represent bonds. For example, the connecting line between B₃ and the nitrogen atom to which R₁ is attached means that B₃, when present, is bonded to this nitrogen.

It will be understood that when some ring members are selected from N, O, and S, the remaining ring members are carbon.

It will be understood that —(CH₂)₀-represents a single bond.

It will be understood that when n=0, L₁ is directly bonded to L_{2.}

It will be understood that when n=0 and Li is a single bond, L₂ is bonded directly to the remainder of the molecule.

It will be understood that when n=0 and L₂ is a single bond, Li is directly bonded to X_{2.}

It will be understood that when n=0 and Li and L₂ are both single bonds, X₂ is bonded directly to the remainder of the molecule.

It will be understood that L₃ may be bonded to any ring member of A, excluding A₁ and A₂. It will be understood that, when L₃ is a single bond, the remainder of the molecule is directly attached to any ring member of A, excluding A₁ and A_{2.}

It will be understood that, when R₃ is a substituted six-membered ring, the para-position to the attachment to B₆ means the ring member opposite the carbon attached to the rest of the molecule. This

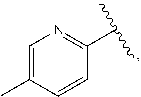 [SMILES: *c1ccc(C)cn1]

is the 4 position when R₃ is substituted phenyl. For example, when R₃ is it has a methyl group at the para-position to the attachment to B_{6.}

It will be understood that, when R₃ is a substituted six-membered ring, the meta-position to the attachment to B₆ means a ring member separated by one other ring member from the carbon attached to the rest of the molecule. This is the 3 position when R₃ is substituted phenyl. For example, when R₃ is

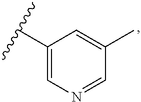 [SMILES: *c1cncc(C)c1]

it has a methyl group at the meta-position to the attachment to B_{6.}

The term “O-linked”, such as in “O-linked hydrocarbon”, means that the hydrocarbon is joined to the remainder of the molecule via an oxygen atom.

In groups such as —(CH₂)₀₋₆-A, “-” denotes the point of attachment of the substituent group to the remainder of the molecule.

It will be understood that “polycycloalkyl” can be adamantyl which is the following structure:

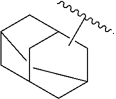

Adamantyl can be optionally substituted as defined for polycycloalkyl above.

It will be understood that, when A does not contain both N and S ring members, A can contain N ring members as long as there are no S ring members and A can contain S ring members as long as there are no N ring members.

It will be understood that R₃ always has at least one substituent as defined above, and may have two substituents as defined above, but may not be unsubstituted.

It will be understood that, when R₃ is not substituted with a 4-methoxy group, R₃ can be substituted otherwise as defined above, but there is no “O—CH₃” substituent at the 4-position, relative to the point of attachment of R₃ to the remainder of the molecule.

It will be understood that, when R₃ is not substituted with a 4-alkoxy group, R₃ can be substituted otherwise as defined above, but there is no alkoxy substituent at the 4-position, relative to the point of attachment of R₃ to the remainder of the molecule.

It will be understood that when any variable (e.g. alkyl) occurs more than once, its definition on each occurrence is independent of every other occurrence.

It will be understood that “pharmaceutically acceptable salts and/or solvates thereof” means “pharmaceutically acceptable salts thereof”, “pharmaceutically acceptable solvates thereof”, and “pharmaceutically acceptable solvates of salts thereof”.

The compounds of the present invention can be provided as mixtures of more than one stereoisomer. When provided as a mixture of stereoisomers, one stereoisomer can be present at a purity >90% relative to the remaining stereoisomers. More specifically, when provided as a mixture of stereoisomers, one stereoisomer can be present at a purity >95% relative to the remaining stereoisomers.

It will be understood that substituents may be named as its free unbonded structure (e.g. piperidine) or by its bonded structure (e.g. piperidinyl). No difference is intended.

It will be understood that the compounds of the invention comprise several substituents. When any of these substituents is defined more specifically herein, the substituents/optional substituents to these groups described above also apply, unless stated otherwise. For example, R₅ can be heteroaryl^{a}, which more specifically can be isoquinolinyl. In this case, isoquinolinyl can be optionally substituted in the same manner as “heteroaryla”.

It will be understood that the term “where possible” means that the group, atom, or substituent in question may be present if it is chemically possible to do so, e.g. does not exceed the valencies of chemically stable compounds. For example, B₁ can, where possible, be substituted with 2 substituents, but only in the instance where B₁ is a carbon centre and is not double-bonded to B₂. This is because, in the instance that B₁ is a nitrogen centre, it is already divalent and only has one spare valency to bond to a substituent or form a double bond with B₂. Similarly, in the instance that B₁ is a carbon centre and double-bonded to B₂, it is already trivalent and only has one spare valency to bond to a substituent.

It will be understood that, unless otherwise specified, a bi-cyclic (or bicyclic) ring system may be a fused, bridged or spiro ring system.

It will be understood that a fused ring system refers to a ring system where two rings in the ring system

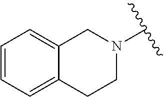 [SMILES: *N1CCc2ccccc2C1]

share two adjacent atoms (i.e., one common covalent bond). For example, is a fused ring system (specifically a fused bicyclic ring system) which can be considered as a benzene ring and a piperidine ring sharing a common bond.

It will be understood that a bridged ring system refers to a ring system having two rings sharing three or

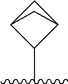 [SMILES: *C1C2CC1C2]

more atoms. For example, is a bridged ring system (specifically a bridged bicyclic ring system) which can be considered as a cyclobutane ring and a second cyclobutane ring joined at a bridge and sharing three common atoms.

It will be understood that, where two different carbon ring members are connected by a bridging —CH₂—

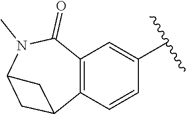 [SMILES: *c1ccc2c(c1)C(=O)N(C)C1CC2C1]

group, a bridged ring system is formed as part of the monocyclic ring A′. For example, contains a bridged ring system wherein two different carbon ring members are connected by a bridging-CH₂-group.

It will be understood that a spiro ring system refers to a ring system where two rings in the ring system

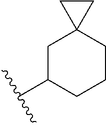 [SMILES: *C1CCCC2(CC2)C1]

share one common atom. For example, is a spiro ring system (specifically a spiro bicyclic ring system) which can be considered as a cyclopropane ring and a cyclohexane ring sharing a common carbon atom.

It will be understood that when any variable (e.g. alkyl) occurs more than once, its definition on each occurrence is independent of every other occurrence.

It will be understood that combinations of substituents and variables are permissible only if such combinations result in stable compounds.
As used herein the term “bradykinin-mediated angioedema” means hereditary angioedema, and any non-hereditary bradykinin-mediated angioedema. For example, “bradykinin-mediated angioedema” encompasses hereditary angioedema and acute bradykinin-mediated angioedema of unknown origin.
As used herein, the term “hereditary angioedema” means any bradykinin-mediated angioedema caused by an inherited genetic dysfunction, fault, or mutation. As a result, the term “HAE” includes at least HAE type 1, HAE type 2, and normal C₁ inhibitor HAE (normal C₁-Inh HAE).
As is clear from the definitions above, and for the avoidance of doubt, it will be understood that “Bx” (for example B₁) is defined above and does not encompass Boron.
As noted above, the invention provides compounds of formula (I),

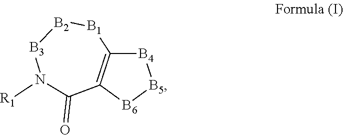

or a tautomer, isomer, stereoisomer (including an enantiomer, a diastereoisomer and a racemic and scalemic mixture thereof), a deuterated analogue, and a pharmaceutically acceptable salt and/or solvate thereof.

The compounds of formula (I) can be compounds of formula (Ia) or (le),

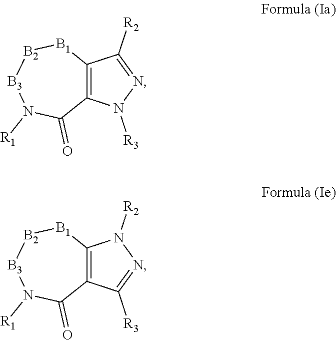

wherein:
B₁ is a carbon centre or nitrogen centre; and either:
(i) B₂ and B₃ together form a single bond between B₁ and the nitrogen atom to which R₁ is attached,
(ii) B₂ is a carbon centre and B₃ is a single bond between B₂ and the nitrogen atom to which R₁ is attached, or
(iii) B₂ and B₃ are both carbon centres wherein, when B₂ is a carbon centre, B₁ is connected to B₂ via either a single or double bond; and wherein, when both B₂ and B₃ are present, B₂ and B₃ are connected via either a single or double bond, or a tautomer, isomer, stereoisomer (including an enantiomer, a diastereoisomer and a racemic and scalemic mixture thereof), a deuterated analogue, and a pharmaceutically acceptable salt and/or solvate thereof.

The compounds of formula (I) can be compounds of formula (Ia),

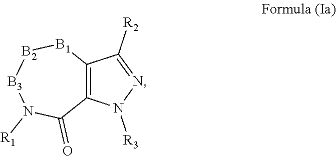

wherein:
B₁ is a carbon centre or nitrogen centre; and either:
(i) B₂ and B₃ together form a single bond between B₁ and the nitrogen atom to which R₁ is attached,
(ii) B₂ is a carbon centre and B₃ is a single bond between B₂ and the nitrogen atom to which R₁ is attached, or
(iii) B₂ and B₃ are both carbon centres wherein, when B₂ is a carbon centre, B₁ is connected to B₂ via either a single or double bond; and wherein, when both B₂ and B₃ are present, B₂ and B₃ are connected via either a single or double bond, or a tautomer, isomer, stereoisomer (including an enantiomer, a diastereoisomer and a racemic and scalemic mixture thereof), a deuterated analogue, and a pharmaceutically acceptable salt and/or solvate thereof.

The compounds of formula (I) can be compounds of formula (Ib), formula (Ic), or formula (Id),

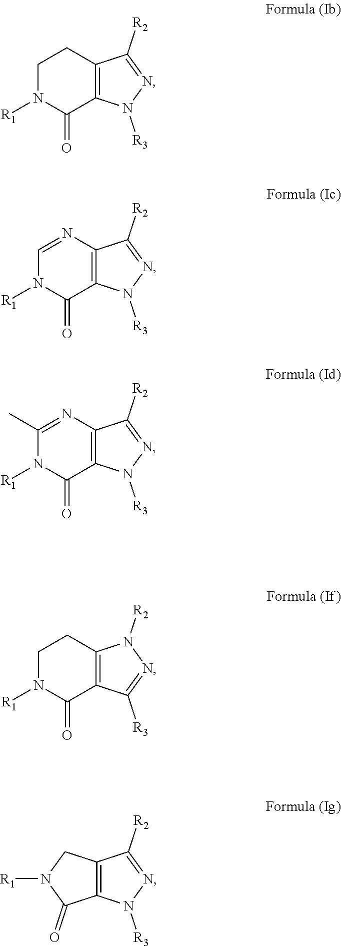

or a tautomer, isomer, stereoisomer (including an enantiomer, a diastereoisomer and a racemic and scalemic mixture thereof), a deuterated analogue, and a pharmaceutically acceptable salt and/or solvate thereof.

The compounds of formula (I) can be compounds of formula (Ib), formula (Ic), or formula (Id),

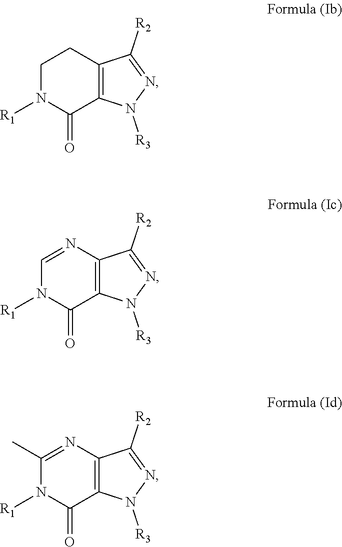

or a tautomer, isomer, stereoisomer (including an enantiomer, a diastereoisomer and a racemic and scalemic mixture thereof), a deuterated analogue, and a pharmaceutically acceptable salt and/or solvate thereof.

The compounds of formula (I) are preferably compounds of formula (Ib),

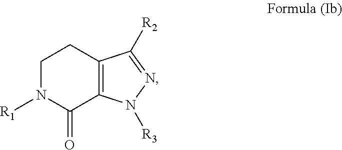

or a tautomer, isomer, stereoisomer (including an enantiomer, a diastereoisomer and a racemic and scalemic mixture thereof), a deuterated analogue, and a pharmaceutically acceptable salt and/or solvate thereof.

The compounds of formula (I) can be compounds of formula (Ic), or formula (Id),

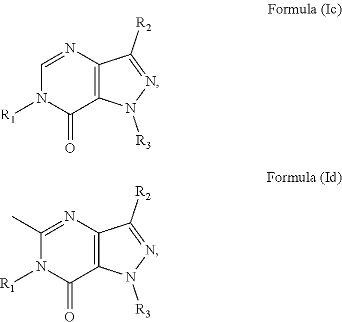

or a tautomer, isomer, stereoisomer (including an enantiomer, a diastereoisomer and a racemic and scalemic mixture thereof), a deuterated analogue, and a pharmaceutically acceptable salt and/or solvate thereof.

When the compound is a compound of formula (Ic) or formula (Id), R₂ may be —CN.

The compounds of formula (I) can be compounds of formula (Ic),

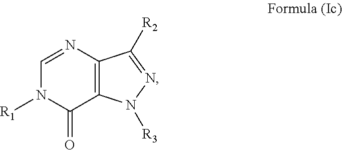

or a tautomer, isomer, stereoisomer (including an enantiomer, a diastereoisomer and a racemic and scalemic mixture thereof), a deuterated analogue, and a pharmaceutically acceptable salt and/or solvate thereof.

The compounds of formula (I) can be compounds of formula (Id),

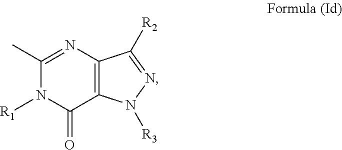

or a tautomer, isomer, stereoisomer (including an enantiomer, a diastereoisomer and a racemic and scalemic mixture thereof), a deuterated analogue, and a pharmaceutically acceptable salt and/or solvate thereof.

The compounds of formula (I) can be compounds of formula (If),

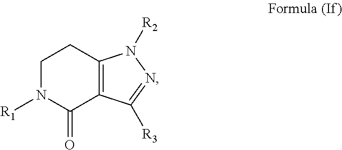

or a tautomer, isomer, stereoisomer (including an enantiomer, a diastereoisomer and a racemic and scalemic mixture thereof), a deuterated analogue, and a pharmaceutically acceptable salt and/or solvate thereof.

The compounds of formula (I) can be compounds of formula (Ig),

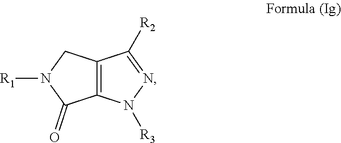

or a tautomer, isomer, stereoisomer (including an enantiomer, a diastereoisomer and a racemic and scalemic mixture thereof), a deuterated analogue, and a pharmaceutically acceptable salt and/or solvate thereof.

Preferably, the compounds of formula (I) are compounds of formula (Ib) or (Id).
As noted above, B₁ is a carbon centre or nitrogen centre; and either:
(i) B₂ and B₃ together form a single bond between B₁ and the nitrogen atom to which R₁ is attached, (ii) B₂ is a carbon centre or a nitrogen centre, and B₃ is a single bond between B₂ and the nitrogen atom to which R₁ is attached,
(iii) B₂ is a carbon centre and B₃ is a nitrogen centre, or
(iv) B₂ and B₃ are both carbon centres, wherein, when B₂ is a carbon centre or nitrogen centre, B₁ is connected to B₂ via either a single or double bond;
wherein, when both B₂ and B₃ are present, B₂ and B₃ are connected via either a single or double bond.
B₁ can be a nitrogen centre. Preferably, B₁ is a carbon centre.
B₁ can be a carbon centre or nitrogen centre and B₂ and B₃ can together form a single bond between B₁ and the nitrogen atom to which R₁ is attached.
B₁ can be a carbon centre and B₂ and B₃ can together form a single bond between B₁ and the nitrogen atom to which R₁ is attached.
B₁ can be a carbon centre or nitrogen centre and B₂ can be a carbon centre or nitrogen centre, wherein B₃ is a single bond between B₂ and the nitrogen atom to which R₁ is attached.
B₁ can be a carbon centre or nitrogen centre and B₂ can be a carbon centre, wherein B₃ is a single bond between B₂ and the nitrogen atom to which R₁ is attached.
B₁ can be a carbon centre and B₂ can be a carbon centre, wherein B₃ is a single bond between B₂ and the nitrogen atom to which R₁ is attached.
B₁ can be a nitrogen centre and B₂ can be a carbon centre, wherein B₃ is a single bond between B₂ and the nitrogen atom to which R₁ is attached.
B₁ can be a carbon centre or nitrogen centre and B₂ can be a carbon centre or nitrogen centre, wherein B₃ is a single bond between B₂ and the nitrogen atom to which R₁ is attached and B₁ is connected to B₂ via a single bond.
B₁ can be a carbon centre or nitrogen centre and B₂ can be a carbon centre or nitrogen centre, wherein
B₃ is a single bond between B₂ and the nitrogen atom to which R₁ is attached and B₁ is connected to B₂ via a double bond.
B₁ can be a carbon centre or nitrogen centre and B₂ can be a carbon centre, wherein B₃ is a single bond between B₂ and the nitrogen atom to which R₁ is attached and B₁ is connected to B₂ via a single bond.
B₁ can be a carbon centre or nitrogen centre and B₂ can be a carbon centre, wherein B₃ is a single bond between B₂ and the nitrogen atom to which R₁ is attached and B₁ is connected to B₂ via a double bond.
B₁ can be a nitrogen centre and B₂ can be a carbon centre, wherein B₃ is a single bond between B₂ and the nitrogen atom to which R₁ is attached and B₁ is connected to B₂ via a double bond.
B₂ and B₃ can together form a single bond between B₁ and the nitrogen atom to which R₁ is attached, or B₂ can be a carbon centre or a nitrogen centre, and B₃ can be a single bond between B₂ and the nitrogen atom to which R₁ is attached.

It can be that either:
(i) B₁ is a carbon centre, and B₂ and B₃ together form a single bond between B₁ and the nitrogen atom to which R₁ is attached,
(ii) B₁ is a carbon centre and B₂ is a carbon centre, and B₁ is connected to B₂ via a single bond, or (iii) B₁ is a nitrogen centre and B₂ is a carbon centre and B₁ is connected to B₂ via a double bond.

Preferably, B₁ is a carbon centre and B₂ is a carbon centre, wherein B₃ is a single bond between B₂ and the nitrogen atom to which R₁ is attached and B₁ is connected to B₂ via a single bond, or B₁ is a nitrogen centre and B₂ is a carbon centre and B₁ is connected to B₂ via a double bond.

Preferably, B₁ is a carbon centre and B₂ is a carbon centre, wherein B₃ is a single bond between B₂ and the nitrogen atom to which R₁ is attached and B₁ is connected to B₂ via a single bond.
B₁ is a carbon centre or nitrogen centre; and it can be that either:
(i) B₂ and B₃ together form a single bond between B₁ and the nitrogen atom to which R₁ is attached,
(ii) B₂ is a carbon centre and B₃ is a single bond between B₂ and the nitrogen atom to which R₁ is attached, or
(iii) B₂ and B₃ are both carbon centres wherein, when B₂ is a carbon centre, B₁ is connected to B₂ via either a single or double bond; and wherein, when both B₂ and B₃ are present, B₂ and B₃ are connected via either a single or double bond.
B₁ can be optionally substituted with 1 or 2 substituents selected from alkyl, cycloalkyl, alkoxy, OH, CF₃, CN, and halo, or 1 oxo substituent.

When B₂ is a carbon centre, B₂ can be optionally substituted with 1 substituent, wherein the substituent is small alkyl, optionally wherein the substituent is methyl.
B₁ can be a carbon centre and B₂ can be a carbon centre, wherein B₃ is a single bond between B₂ and the nitrogen atom to which R₁ is attached and B₁ is connected to B₂ via a single bond, or B₁ can be a nitrogen centre and B₂ can be a carbon centre wherein B₁ is connected to B₂ via a double bond; wherein B₁ is optionally substituted with 1 or 2 substituents selected from alkyl, cycloalkyl, alkoxy, OH, CF₃, CN, and halo, or 1 oxo substituent.
B₁ can be a carbon centre and B₂ can be a carbon centre, wherein B₃ is a single bond between B₂ and the nitrogen atom to which R₁ is attached and B₁ is connected to B₂ via a single bond, and wherein B₁ is optionally substituted with 1 or 2 substituents selected from alkyl, cycloalkyl, alkoxy, OH, CF₃, CN, and halo, or 1 oxo substituent.
B₁ can be a nitrogen centre and B₂ can be a carbon centre, wherein B₃ is a single bond between B₂ and the nitrogen atom to which R₁ is attached and B₁ is connected to B₂ via a double bond, and wherein B₂ is optionally substituted 1 substituent, wherein the substituent is small alkyl, optionally wherein the substituent is methyl.
As noted above, either:
(i) B₄ is N(R₂), B₅ is N, and B₆ is C(R₃), and B₄ is connected to B₅ via a single bond and B₅ is connected to B₆ via a double bond;
(ii) B₄ is C(R₂), B₅ is N, and B₆ is N(R₃), and B₄ is connected to B₅ via a double bond and B₅ is connected to B₆ via a single bond; or
(iii) B₄ is C(R₂), B₅ is C (H), and B₆ is N(R₃), and B₄ is connected to B₅ via a double bond and B₅ is connected to B₆ via a single bond.

Preferably, B₄ is N(R₂), B₅ is N, and B₆ is C(R₃), wherein B₄ is connected to Bs via a single bond and B₅ is connected to B₆ via a double bond, or B₄ is C(R₂), Bs is N, and B₆ is N(R₃), wherein B₄ is connected to B₅ via a double bond and B₅ is connected to B₆ via a single bond.

Preferably, B₄ is C(R₂), B₅ is N, and B₆ is N(R₃), wherein B₄ is connected to B₅ via a double bond and B₅ is connected to B₆ via a single bond.
B₁ can be a carbon centre and B₂ can be a carbon centre, wherein B₃ is a single bond between B₂ and the nitrogen atom to which R₁ is attached and B₁ is connected to B₂ via a single bond, or B₁ can be a nitrogen centre and B₂ can be a carbon centre wherein B₁ is connected to B₂ via a double bond; and B₄ can be N(R₂), B₅ can be N, and B₆ can be C(R₃), wherein B₄ is connected to B₅ via a single bond and B₅ is connected to B₆ via a double bond, or B₄ can be C(R₂), B₅ can be N, and B₆ can be N(R₃), wherein B₄ is connected to B₅ via a double bond and B₅ is connected to B₆ via a single bond.
B₁ can be a carbon centre and B₂ can be a carbon centre, wherein B₃ is a single bond between B₂ and the nitrogen atom to which R₁ is attached and B₁ is connected to B₂ via a single bond, and B₄ can be C(R₂), B₅ can be N, and B₆ can be N(R₃), wherein B₄ is connected to B₅ via a double bond and B₅ is connected to B₆ via a single bond.
As noted above, R₂ is selected from H, —(CH₂)₀₋₃—C(═O)—N(R₄)(R₅), —C(═O)—O—R₆, —(CH₂)₀₋₃—NR₁₄—C(═O) R₁₅, —(CH₂)₀₋₃—NR₃₄—S(═O)₂R₃₅, alkyl, cycloalkyl, heterocycloalkyl, alkoxy, —OH, —CF₃, halo, aryl, heteroaryl, —CH₂—O—R₁₆ and —CN;
wherein R₄ is H, alkyl, cycloalkyl, and heterocycloalkyl, and
R₅ is selected from alkyl, —OH, —S(═O)₂-(small alkyl), —(CH₂)₁₋₃—(C═O)—OR₃₃, —(CH₂)₁₋₃—(C═O)—N(R₃₆)(R₃₇), —(CH₂)₀₋₃-cycloalkyl^{b}, —(CH₂)₀₋₃-heterocycloalkyl, —(CH(alkyl))-heterocycloalkyl, —(CH₂)₀₋₃-polycycloalkyl, —(CH₂)₀₋₃-aryl, —(C(H)(phenyl))-alkyl, —(CH₂)₀₋₃heteroaryl^{a}, —(C(H)(heteroaryl))-alkyl, —(C(H)(alkyl))—(C═O)—OR₃₈, —(C(H)(alkyl))—(C═O)—N(R₃₉)(R₄₀), —(C(H)(heteroaryl))—(C═O)—OR₂₈, and —(C(H)(heteroaryl))—(C═O)—N(R₄₁)(R₄₂);
or wherein R₄ and R₅ together with the nitrogen atom to which they are attached form a carbon-containing 4-, 5-, 6-, or 7-membered heterocyclic ring which may be saturated or unsaturated with 1 or 2 double bonds, optionally containing one or two additional heteroatoms selected from N, O, and S, wherein the ring may be optionally substituted with 1, 2, or 3 substituents selected from oxo, alkyl, alkoxy, OH, halo and CF_{3;}
R₂₈, R₃₃ R₃₆, R₃₇, R₃₈, R₃₉, R₄₀, R₄₁, and R₄₂ are each independently selected from H and small alkyl; R₆ is selected from H, alkyl, —(CH₂)₀₋₃-cycloalkyl, —(CH₂)₀₋₃-heterocycloalkyl, —(CH(alkyl))-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, and —(C(H)(heteroaryl))-alkyl;
R₁₄ is selected from H, alkyl, cycloalkyl, and heterocycloalkyl;
R 15 is selected from H, alkyl, —(CH₂)₀₋₃-cycloalkyl, —(CH₂)₀₋₃-heterocycloalkyl, —(CH(alkyl))-heterocycloalkyl, —(C(H)(phenyl))-alkyl, —(CH₂)₀₋₃heteroaryl^{a}, and —(C(H)(heteroaryl))-alkyl;

—(CH₂)₀₋₃-aryl,
R₁₆ is selected from H, alkyl, cycloalkyl, —(CH₂)₁₋₃—(C═O)—OH, heterocycloalkyl, and —(CH₂)₀₋₃heteroaryl;
R₃₄ is selected from H, alkyl, cycloalkyl, and heterocycloalkyl;
R₃₅ is selected from H, alkyl, —(CH₂)₀₋₃-cycloalkyl, —(CH₂)₀₋₃-heterocycloalkyl, —(CH(alkyl))-heterocycloalkyl, —(CH₂)₀₋₃-aryl, —(C(H)(phenyl))-alkyl, —(CH₂)₀₋₃heteroaryl^{a}, and —(C(H)(heteroaryl))-alkyl.
R₂ can be selected from H, —(CH₂)₀₋₃—C(═O)—N(R₄)(R₅), —C(═O)—O—R₆, —(CH₂)₀₋₃—NR₁₄—C(═O) R₁₅, —(CH₂)₀₋₃—NR₃₄—S(═O)₂R₃₅, alkyl, cycloalkyl, heterocycloalkyl, alkoxy, —OH, —CF₃, halo, aryl, heteroaryl, —CH₂—O—R₁₆ and —CN;
wherein R₄ is H, alkyl, cycloalkyl, and heterocycloalkyl, and
R₅ is selected from alkyl, —OH, —S(═O)₂-(small alkyl), —(CH₂)₁₋₃—(C═O)—OR₃₃, —(CH₂)₁₋₃—(C═O)—N(R₃₆)(R₃₇), —(CH₂)₀₋₃-cycloalkyl^{a}, —(CH₂)₀₋₃-heterocycloalkyl, —(CH(alkyl))-heterocycloalkyl, —(CH₂)₀₋₃-polycycloalkyl, —(CH₂)₀₋₃-aryl, —(C(H)(phenyl))-alkyl, —(CH₂)₀₋₃heteroaryl^{a}, —(C(H)(heteroaryl)-alkyl, —(C(H)(alkyl)—(C═O)—OR₃₈, —(C(H)(alkyl)-(C═O)—N(R₃₉)(R₄₀) , —(C(H)(heteroaryl))—(C═O)—OR₂₈, and —(C(H)(heteroaryl))—(C═O)—N(R₄₁)(R₄₂);
or wherein R₄ and R₅ together with the nitrogen atom to which they are attached form a carbon-containing 4-, 5-, 6-, or 7-membered heterocyclic ring which may be saturated or unsaturated with 1 or 2 double bonds, optionally containing one or two additional heteroatoms selected from N, O, and S, wherein the ring may be optionally substituted with 1, 2, or 3 substituents selected from oxo, alkyl, alkoxy, OH, halo and CF_{3;}
R₂₈, R₃₃ R₃₆, R₃₇, R₃₈, R₃₉, R₄₀, R₄₁, and R₄₂ are each independently selected from H and small alkyl; R₆ is selected from H, alkyl, —(CH₂)₀₋₃-cycloalkyl, —(CH₂)₀₋₃-heterocycloalkyl, —(CH(alkyl))-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, and —(C(H)(heteroaryl))-alkyl; R₁₄ is selected from H, alkyl, cycloalkyl, and heterocycloalkyl;
R₁₅ is selected from H, alkyl, —(CH₂)₀₋₃-cycloalkyl, —(CH₂)₀₋₃-heterocycloalkyl, —(CH(alkyl))-heterocycloalkyl, —(CH₂)₀₋₃-aryl, —(C(H)(phenyl))-alkyl, —(CH₂)₀₋₃heteroaryl^{a}, and —(C(H)(heteroaryl))-alkyl; R₁₆ is selected from H, alkyl, cycloalkyl, —(CH₂)₁₋₃—(C═O)—OH, heterocycloalkyl, and —(CH₂)₀₋₃heteroaryl; R₃₄ is selected from H, alkyl, cycloalkyl, and heterocycloalkyl;
R₃₅ is selected from H, alkyl, —(CH₂)₀₋₃-cycloalkyl, —(CH₂)₀₋₃-heterocycloalkyl, —(CH(alkyl))-heterocycloalkyl, —(CH₂)₀₋₃-aryl, —(C(H)(phenyl))-alkyl, —(CH₂)₀₋₃heteroaryl^{a}, and —(C(H)(heteroaryl))-alkyl.
R₂ can be selected from H, —(CH₂)₀₋₃—C(═O)—N(R₄)(R₅), —C(═O)—O—R₆, —(CH₂)₀₋₃—NR₁₄—C(═O) R₁₅, —(CH₂)₀₋₃—NR₃₄—S(═O)₂R₃₅, alkyl, cycloalkyl, heterocycloalkyl, alkoxy, —OH, —CF₃, halo, aryl, heteroaryl, —CH₂—O—R₁₆ and —CN;
wherein R₄ is H, alkyl, cycloalkyl, and heterocycloalkyl, and

R₅ is selected from alkyl, —(CH₂)₀₋₃-cycloalkyl, —(CH₂)₀₋₃-heterocycloalkyl, —(CH(alkyl))-heterocycloalkyl, —(CH₂)₀₋₃-polycycloalkyl, —(CH₂)₀₋₃-aryl, —(C(H)(phenyl))-alkyl, —(CH₂)₀₋₃heteroaryl^{a}, and —(C(H)(heteroaryl))-alkyl;
or wherein R₄ and R₅ together with the nitrogen atom to which they are attached form a carbon-containing 4-, 5-, 6-, or 7-membered heterocyclic ring which may be saturated or unsaturated with 1 or 2 double bonds, optionally containing one or two additional heteroatoms selected from N, O, and S, wherein the ring may be optionally substituted with 1, 2, or 3 substituents selected from oxo, alkyl, alkoxy, OH, halo and CF_{3;}
R₆ is selected from H, alkyl, —(CH₂)₀₋₃-cycloalkyl, —(CH₂)₀₋₃-heterocycloalkyl, —(CH(alkyl))-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, and —(C(H)(heteroaryl))-alkyl;
R₁₄ is selected from H, alkyl, cycloalkyl, and heterocycloalkyl;
R₁₅ is selected from H, alkyl, —(CH₂)₀₋₃-cycloalkyl, —(CH₂)₀₋₃-heterocycloalkyl, —(CH(alkyl))-heterocycloalkyl, —(CH₂)₀₋₃-aryl, —(C(H)(phenyl))-alkyl, —(CH₂)₀₋₃heteroaryl^{a}, and —(C(H)(heteroaryl))-alkyl; and
R₁₆ is selected from H, alkyl, cycloalkyl, and heterocycloalkyl;
R₃₄ is selected from H, alkyl, cycloalkyl, and heterocycloalkyl;
R₃₅ is selected from H, alkyl, —(CH₂)₀₋₃-cycloalkyl, —(CH₂)₀₋₃-heterocycloalkyl, —(CH(alkyl))-heterocycloalkyl, —(CH₂)₀₋₃-aryl, —(C(H)(phenyl))-alkyl, —(CH₂)₀₋₃heteroaryl^{a}, and —(C(H)(heteroaryl))-alkyl.
R₂ can be selected from H, —(CH₂)₀₋₃—C(═O)—N(R₄)(R₅), —C(═O)—O—R₆, —(CH₂)₀₋₃—NR₁₄—C(═O) R₁₅, alkyl, cycloalkyl, heterocycloalkyl, alkoxy, —OH, —CF₃, halo, aryl, heteroaryl, —CH₂—O—R₁₆ and —CN;
wherein R₄ is H, alkyl, cycloalkyl, and heterocycloalkyl, and
R₅ is selected from alkyl, —(CH₂)₀₋₃-cycloalkyl, —(CH₂)₀₋₃-heterocycloalkyl, —(CH₂)₀₋₃-polycycloalkyl, —(CH₂)₀₋₃-aryl, —(C(H)(phenyl))-alkyl, —(CH₂)₀₋₃heteroaryl^{a}, and —(C(H)(heteroaryl))-alkyl;
or wherein R₄ and R₅ together with the nitrogen atom to which they are attached form a carbon-containing 4-, 5-, 6-, or 7-membered heterocyclic ring which may be saturated or unsaturated with 1 or 2 double bonds, optionally containing one or two additional heteroatoms selected from N, O, and S, wherein the ring may be optionally substituted with 1, 2, or 3 substituents selected from oxo, alkyl, alkoxy, OH, halo and CF_{3;}
R₆ is selected from H, alkyl, —(CH₂)₀₋₃-cycloalkyl, —(CH₂)₀₋₃-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, and —(C(H)(heteroaryl))-alkyl;
R₁₄ is selected from H, alkyl, cycloalkyl, and heterocycloalkyl;
R₁₅ is selected from H, alkyl, —(CH₂)₀₋₃-cycloalkyl, —(CH₂)₀₋₃-heterocycloalkyl, —(CH₂)₀₋₃-aryl, —(C(H)(phenyl))-alkyl, —(CH₂)₀₋₃heteroaryl^{a}, and —(C(H)(heteroaryl))-alkyl; and
R₁₆ is selected from H, alkyl, cycloalkyl, and heterocycloalkyl.
R₂ can be selected from —(CH₂)₀₋₃—C(═O)—N(R₄)(R₅), —C(═O)—O—R₆, —(CH₂)₀₋₃—NR₁₄—C(═O) R₁₅, —(CH₂)₀₋₃—NR₃₄—S(═O)₂R₃₅, alkyl, cycloalkyl, heterocycloalkyl, alkoxy, —OH, —CF₃, halo, aryl, heteroaryl, —CH₂—O—R₁₆ and —CN.
R₂ can be selected from H, —(CH₂)₀₋₃—C(═O)—N(R₄)(R₅), —C(═O)—O—R₆, —(CH₂)₀₋₃—NR₁₄—C(═O) R₁₅, —(CH₂)₀₋₃—NR₃₄—S(═O)₂R₃₅, alkyl, cycloalkyl, alkoxy, —OH, —CF₃, halo, aryl, heteroaryl, —CH₂—O—R₁₆ and —CN.
R₂ can be selected from H, —(CH₂)₀₋₃—C(═O)—N(R₄)(R₅), —C(═O)—O—R₆, —(CH₂)₀₋₃—NR₁₄—C(═O) R₁₅, —(CH₂)₀₋₃—NR₃₄—S(═O)₂R₃₅, alkyl, cycloalkyl, heterocycloalkyl, alkoxy, —OH, —CF₃, halo, aryl, —CH₂—O—R₁₆ and —CN.
R₂ can be selected from H, —(CH₂)₀₋₃—C(═O)—N(R₄)(R₅), —(CH₂)₀₋₃—NR₁₄—C(═O) R₁₅, —(CH₂)₀₋₃—NR₃₄—S(═O)₂R₃₅, alkyl, cycloalkyl, heterocycloalkyl, alkoxy, —OH, —CF₃, halo, aryl, heteroaryl, —CH₂—O—R₁₆ and —CN.
R₂ can be selected from H, —(CH₂)₀₋₃—C(═O)—N(R₄)(R₅), —C(═O)—O—R₆, —(CH₂)₀₋₃—NR₁₄—C(═O) R₁₅, —(CH₂)₀₋₃—NR₃₄—S(═O)₂R₃₅, alkyl, cycloalkyl, heterocycloalkyl, alkoxy, —OH, —CF₃, halo, aryl, heteroaryl, and —CH₂—O—R_{16.}
R₂ can be selected from H, —(CH₂)₀₋₃—C(═O)—N(R₄)(R₅), —C(═O)—O—R₆, —(CH₂)₀₋₃—NR₁₄—C(═O) R₁₅, —(CH₂)₀₋₃—NR₃₄—S(═O)₂R₃₅, alkyl, cycloalkyl, heterocycloalkyl, alkoxy, —OH, halo, aryl, heteroaryl, —CH₂—O—R₁₆, and —CN.
R₂ can be selected from —(CH₂)₀₋₃—C(═O)—N(R₄)(R₅), —C(═O)—O—R₆, —(CH₂)₀₋₃—NR₁₄—C(═O) R₁₅, alkyl, cycloalkyl, heterocycloalkyl, alkoxy, —OH, —CF₃, halo, aryl, heteroaryl, —CH₂—O—R₁₆ and —CN.
R₂ can be selected from H, —(CH₂)₀₋₃—C(═O)—N(R₄)(R₅), —(CH₂)₀₋₃—NR₁₄—C(═O) R₁₅, alkyl, cycloalkyl, heterocycloalkyl, alkoxy, —OH, —CF₃, halo, aryl, heteroaryl, —CH₂—O—R₁₆ and —CN.
R₂ can be selected from H, —(CH₂)₀₋₃—C(═O)—N(R₄)(R₅), —C(═O)—O—R₆, —(CH₂)₀₋₃—NR₁₄—C(═O) R₁₅, alkyl, cycloalkyl, heterocycloalkyl, alkoxy, —OH, —CF₃, halo, aryl, heteroaryl, and —CH₂—O—R_{16.}
R₂ can be selected from H, —(CH₂)₀₋₃—C(═O)—N(R₄)(R₅), —C(═O)—O—R₆, —(CH₂)₀₋₃—NR₁₄—C(═O) R₁₅, alkyl, cycloalkyl, heterocycloalkyl, alkoxy, —OH, halo, aryl, heteroaryl, —CH₂—O—R₁₆, and —CN.
R₂ can be selected from —(CH₂)₀₋₃—C(═O)—N(R₄)(R₅), —(CH₂)₀₋₃—NR₁₄—C(═O) R₁₅, —(CH₂)₀₋₃—NR₃₄—S(═O)₂R₃₅, alkyl, cycloalkyl, heterocycloalkyl, alkoxy, —OH, —CF₃, halo, aryl, heteroaryl, —CH₂—O—R₁₆ and —CN.
R₂ can be selected from —(CH₂)₀₋₃—C(═O)—N(R₄)(R₅), —C(═O)—O—R₆, —(CH₂)₀₋₃—NR₁₄—C(═O) R₁₅, —(CH₂)₀₋₃—NR₃₄—S(═O)₂R₃₅, alkyl, cycloalkyl, heterocycloalkyl, alkoxy, —OH, —CF₃, halo, aryl, heteroaryl, and —CH₂—O—R_{16.}
R₂ can be selected from —(CH₂)₀₋₃—C(═O)—N(RA) (R₅), —C(═O)—O—R₆, —(CH₂)₀₋₃—NR₁₄—C(═O) R₁₅, —(CH₂)₀₋₃—NR₃₄—S(═O)₂R₃₅, alkyl, cycloalkyl, heterocycloalkyl, alkoxy, —OH, halo, aryl, heteroaryl, —CH₂—O—R₁₆, and —CN.
R₂ can be selected from H, —(CH₂)₀₋₃—C(═O)—N(R₄)(R₅), —(CH₂)₀₋₃—NR₁₄—C(═O) R₁₅, —(CH₂)₀₋₃—NR₃₄—S(═O)₂R₃₅, alkyl, cycloalkyl, heterocycloalkyl, alkoxy, —OH, —CF₃, halo, aryl, heteroaryl, and —CH₂—O—R_{16.}
R₂ can be selected from H, —(CH₂)₀₋₃—C(═O)—N(R₄)(R₅), —(CH₂)₀₋₃—NR₁₄—C(═O) R₁₅, —(CH₂)₀₋₃—NR₃₄—S(═O)₂R₃₅, alkyl, cycloalkyl, heterocycloalkyl, alkoxy, —OH, halo, aryl, heteroaryl, —CH₂—O—R₁₆, and —CN.
R₂ can be selected from H, —(CH₂)₀₋₃—C(═O)—N(R₄)(R₅), —C(═O)—O—R₆, —(CH₂)₀₋₃—NR₁₄—C(═O) R₁₅, —(CH₂)₀₋₃—NR₃₄—S(═O)₂R₃₅, alkyl, cycloalkyl, heterocycloalkyl, alkoxy, —OH, halo, aryl, heteroaryl, and —CH₂—O—R_{16.}
R₂ can be selected from —(CH₂)₀₋₃—C(═O)—N(R₄)(R₅), —(CH₂)₀₋₃—NR₁₄—C(═O) R₁₅, alkyl, cycloalkyl, heterocycloalkyl, alkoxy, —OH, —CF₃, halo, aryl, heteroaryl, —CH₂—O—R₁₆ and —CN.
R₂ can be selected from —(CH₂)₀₋₃—C(═O)—N(R₄)(R₅), —C(═O)—O—R₆, —(CH₂)₀₋₃—NR₁₄—C(═O) R₁₅, alkyl, cycloalkyl, heterocycloalkyl, alkoxy, —OH, —CF₃, halo, aryl, heteroaryl, and —CH₂—O—R_{16.}
R₂ can be selected from H, —(CH₂)₀₋₃—C(═O)—N(R₄)(R₅), —(CH₂)₀₋₃—NR₁₄—C(═O) R₁₅, alkyl, cycloalkyl, heterocycloalkyl, alkoxy, —OH, —CF₃, halo, aryl, heteroaryl, and —CH₂—O—R_{16.}
R₂ can be selected from H, —(CH₂)₀₋₃—C(═O)—N(R₄)(R₅), —(CH₂)₀₋₃—NR₁₄—C(═O) R₁₅, alkyl, cycloalkyl, heterocycloalkyl, alkoxy, —OH, halo, aryl, heteroaryl, —CH₂—O—R₁₆, and —CN.
R₂ can be selected from H, —(CH₂)₀₋₃—C(═O)—N(R₄)(R₅), —C(═O)—O—R₆, —(CH₂)₀₋₃—NR₁₄—C(═O) R₁₅, alkyl, cycloalkyl, heterocycloalkyl, alkoxy, —OH, halo, aryl, heteroaryl, and —CH₂—O—R_{16.}
R₂ can be selected from —(CH₂)₀₋₃—C(═O)—N(R₄)(R₅), —(CH₂)₀₋₃—NR₁₄—C(═O) R₁₅, —(CH₂)₀₋₃—NR₃₄—S(═O)₂R₃₅, alkyl, cycloalkyl, heterocycloalkyl, alkoxy, —OH, —CF₃, halo, aryl, heteroaryl, and —CH₂—O—R_{16.}
R₂ can be selected from —(CH₂)₀₋₃—C(═O)—N(R₄)(R₅), —(CH₂)₀₋₃—NR₁₄—C(═O) R₁₅, —(CH₂)₀₋₃—NR₃₄—S(═O)₂R₃₅, alkyl, cycloalkyl, heterocycloalkyl, alkoxy, —OH, halo, aryl, heteroaryl, —CH₂—O—R₁₆, and —CN.
R₂ can be selected from —(CH₂)₀₋₃—C(═O)—N(R₄)(R₅), —C(═O)—O—R₆, —(CH₂)₀₋₃—NR₁₄—C(═O) R₁₅, —(CH₂)₀₋₃—NR₃₄—S(═O)₂R₃₅, alkyl, cycloalkyl, heterocycloalkyl, alkoxy, —OH, halo, aryl, heteroaryl, and —CH₂—O—R_{16.}
R₂ can be selected from —(CH₂)₀₋₃—C(═O)—N(R₄)(R₅), —(CH₂)₀₋₃—NR₁₄—C(═O) R₁₅, alkyl, cycloalkyl, heterocycloalkyl, alkoxy, —OH, —CF₃, halo, aryl, heteroaryl, and —CH₂—O—R_{16.}
R₂ can be selected from —(CH₂)₀₋₃—C(═O)—N(R₄)(R₅), —(CH₂)₀₋₃—NR₁₄—C(═O) R₁₅, alkyl, cycloalkyl, heterocycloalkyl, alkoxy, —OH, halo, aryl, heteroaryl, —CH₂—O—R₁₆, and —CN.
R₂ can be selected from —(CH₂)₀₋₃—C(═O)—N(R₄)(R₅), —C(═O)—O—R₆, —(CH₂)₀₋₃—NR₁₄—C(═O) R₁₅, alkyl, cycloalkyl, heterocycloalkyl, alkoxy, —OH, halo, aryl, heteroaryl, and —CH₂—O—R_{16.}
R₂ can be selected from H, —(CH₂)₀₋₃—C(═O)—N(R₄)(R₅), —(CH₂)₀₋₃—NR₁₄—C(═O) R₁₅, —(CH₂)₀₋₃—NR₃₄—S(═O)₂R₃₅, alkyl, cycloalkyl, heterocycloalkyl, alkoxy, —OH, halo, aryl, heteroaryl, and —CH₂—O—R_{16.}
R₂ can be selected from H, —(CH₂)₀₋₃—C(═O)—N(R₄)(R₅), —(CH₂)₀₋₃—NR₁₄—C(═O) R₁₅, alkyl, cycloalkyl, heterocycloalkyl, alkoxy, —OH, halo, aryl, heteroaryl, and —CH₂—O—R_{16.}
R₂ can be selected from —(CH₂)₀₋₃—C(═O)—N(R₄)(R₅), —(CH₂)₀₋₃—NR₁₄—C(═O) R₁₅, —(CH₂)₀₋₃—NR₃₄—S(═O)₂R₃₅, alkyl, cycloalkyl, heterocycloalkyl, alkoxy, —OH, halo, aryl, heteroaryl, and —CH₂—O—R_{16.}
R₂ can be selected from —(CH₂)₀₋₃—C(═O)—N(R₄)(R₅), —(CH₂)₀₋₃—NR₁₄—C(═O) R₁₅, alkyl, cycloalkyl, heterocycloalkyl, alkoxy, —OH, halo, aryl, heteroaryl, and —CH₂—O—R_{16.}
R₂ can be selected from H, —(CH₂)₀₋₃—C(═O)—N(R₄)(R₅), —C(═O)—O—R₆,—CH₂—NH—C(═O) CH₃,—CH₂—NH—S(═O)₂R₃₅, —CH₂—O—R₁₆,—CH₂—OH, —CN, and

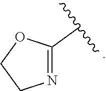 [SMILES: *C1=NCCO1]

R₂ can be selected from —(CH₂)₀₋₃—C(═O)—N(R₄)(R₅), —C(═O)—O—R₆,—CH₂—NH—C(═O) CH₃,—CH₂—NH—S(═O)₂R₃₅,

—CH₂—O—R₁₆,—CH₂—OH, —CN, and

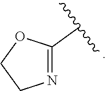 [SMILES: *C1=NCCO1]

R₂ can be selected from —(CH₂)₀₋₃—C(═O)—N(R₄)(R₅), —C(═O)—O—R₆,—CH₂—NH—C(═O) CH₃,
—CH₂—NH—S(═O)₂(cyclopropyl),—CH₂—O—R₁₆,—CH₂—OH, —CN, and

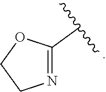 [SMILES: *C1=NCCO1]

R₂ can be selected from —(CH₂)₀₋₃—C(═O)—N(R₄)(R₅), —C(═O)—O—R₆,—CH₂—NH—S(═O)₂(cyclopropyl),—CH₂—O—CH₃, —CH₂—OH, and —CN.
R₂ can be selected from H, —(CH₂)₀₋₃—C(═O)—N(R₄)(R₅), —C(═O)—O—R₆,—CH₂—NH—C(═O) CH₃,—CH₂—NH—S(═O)₂R₃₅,

—CH₂—OH, —CH₂—O—R₁₆, and

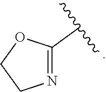 [SMILES: *C1=NCCO1]

R₂ can be selected from —(CH₂)₀₋₃—C(═O)—N(R₄)(R₅), —C(═O)—O—R₆,—CH₂—NH—C(═O) CH₃,—CH₂—NH—S(═O)₂R₃₅,

—CH₂—OH, —CH₂—O—R₁₆, and

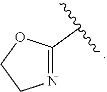 [SMILES: *C1=NCCO1]

R₂ can be selected from —(CH₂)₀₋₃—C(═O)—N(R₄)(R₅), —C(═O)—O—R₆,—CH₂—NH—C(═O) CH₃,

—CH₂—NH—S(═O)₂(cyclopropyl),—CH₂—OH, —CH₂—O—R₁₆, and

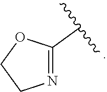 [SMILES: *C1=NCCO1]

R₂ can be selected from —(CH₂)₀₋₃—C(═O)—N(R₄)(R₅), —C(═O)—O—R₆,—CH₂—NH—S(═O)>(cyclopropyl),—CH₂—OH, and —CH₂—O—CH_{3.}
R₂ can be selected from H, —(CH₂)₀₋₃—C(═O)—N(R₄)(R₅),—CH₂—NH—C(═O) CH₃,—CH₂—NH—S(═O)₂R₃₅,—CH₂—O—R₁₆,

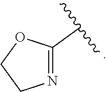 [SMILES: *C1=NCCO1]

—CH₂—OH, —CN, and
R₂ can be selected from —(CH₂)₀₋₃—C(═O)—N(R₄)(R₅),—CH₂—NH—C(═O) CH₃,—CH₂—NH—S(═O)₂(cyclopropyl),

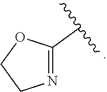 [SMILES: *C1=NCCO1]

—CH₂—O—R₁₆,—CH₂—OH, —CN, and
R₂ can be selected from —(CH₂)₀₋₃—C(═O)—N(R₄)(R₅),—CH₂—NH—C(═O) CH₃,—CH₂—NH—S(═O)₂(cyclopropyl),

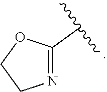 [SMILES: *C1=NCCO1]

—CH₂—O—R₁₆,—CH₂—OH, —CN, and
R₂ can be selected from —(CH₂)₀₋₃—C(═O)—N(R₄)(R₅),—CH₂—NH—S(═O)₂(cyclopropyl),—CH₂—O—CH₃,—CH₂—OH, and —CN.
Rz can be selected from H, —(CH₂)₀₋₃—C(═O)—N(RA) (R₅),—CH₂—NH—C(═O) CH₃,—CH₂—NH—S(═O)₂R₃₅,—CH₂—O—R₁₆,

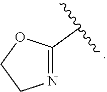 [SMILES: *C1=NCCO1]

—CH₂—OH, and
Rz can be selected from —(CH₂)₀₋₃—C(═O)—N(R₄)(R₅),—CH₂—NH—C(═O) CH₃,—CH₂—NH—S(═O)₂R₃₅,—CH₂—O—R₁₆,

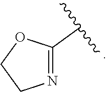 [SMILES: *C1=NCCO1]

—CH₂—OH, and
R₂ can be selected from —(CH₂)₀₋₃—C(═O)—N(Ra) (R₅),—CH₂—NH—C(═O) CH₃,—CH₂—NH—S(═O)> (cyclopropyl),

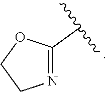 [SMILES: *C1=NCCO1]

—CH₂—O—R₁₆₃,—CH₂—OH, and
R₂ can be selected from —(CH₂)₀₋₃—C(═O)—N(R₄)(R₅),—CH₂—NH—S(═O)₂(cyclopropyl),—CH₂—O—CH₃, and —CH₂—OH.
R₂ can be selected from H, —C(═O)—N(Ra) (R₅), —C(═O)—O—R₆,—CH₂—NH—C(═O) CH₃,—CH₂—NH—S(═O)₂R₃₅,

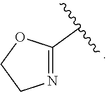 [SMILES: *C1=NCCO1]

—CH₂—O—R₁₆,—CH₂—OH, —CN, and

Preferably, R₂ can be selected from —C(═O)—N(Ra) (R₅), —C(═O)—O—R₆,—CH₂—NH—C(═O) CH₃,—CH₂—NH—S(═O)₂R₃₅,

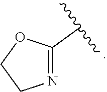 [SMILES: *C1=NCCO1]

—CH₂—O—R₁₆,—CH₂—OH, —CN, and
R₂ can be selected from —C(═O)—N(R₄)(R₅), —C(═O)—O—R₆,—CH₂—NH—C(═O) CH₃,

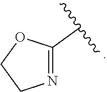 [SMILES: *C1=NCCO1]

—CH₂—NH—S(═O)₂(cyclopropyl),—CH₂—O—R₁₆,—CH₂—OH, —CN, and
R₂ can be selected from —C(═O)—N(R₄)(R₅), —C(═O)—O—R₆,—CH₂—NH—S(═O) z (cyclopropyl),—CH₂—O—CH₃,—CH₂—OH, and —CN.
R₂ can be selected from H, —C(═O)—N(Ra) (R₅), —C(═O)—O—R₆,—CH₂—NH—C(═O) CH₃,—CH₂—NH—S(═O)₂R₃₅,

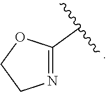 [SMILES: *C1=NCCO1]

—CH₂—O—R₁₆,—CH₂—OH, and
R₂ is selected from —C(═O)—N(RA) (R₅), —C(═O)—O—R₆,—CH₂—NH—C(═O) CH₃,—CH₂—NH—S(═O)₂R₃₅,

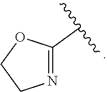 [SMILES: *C1=NCCO1]

—CH₂—O—R₁₆,—CH₂—OH, and
R₂ can be selected from —C(═O)—N(RA) (R₅), —C(═O)—O—R₆,—CH₂—NH—C(═O) CH₃,

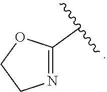 [SMILES: *C1=NCCO1]

—CH₂—NH—S(═O)₂(cyclopropyl),—CH₂—O—R₁₆,—CH₂—OH, and
R₂ can selected from —C(═O)—N(R₄)(R₅), —C(═O)—O—R₆,—CH₂—NH—S(═O)₂(cyclopropyl), —CH₂—O—CH₃, and —CH₂—OH.
R₂ can be selected from H, —C(═O)—N(R₄)(R₅),—CH₂—NH—C(═O) CH₃,—CH₂—NH—S(═O)₂R₃₅,—CH₂—O—R₁₆,—CH₂—OH,

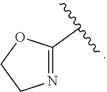 [SMILES: *C1=NCCO1]

—CN, and
R₂ can be selected from —C(═O)—N(R₄)(R₅),—CH₂—NH—C(═O) CH₃,—CH₂—NH—S(═O)₂R₃₅,—CH₂—O—R₁₆,—CH₂—OH, —CN,

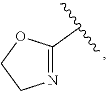 [SMILES: *C1=NCCO1]

and
R₂ can be selected from —C(═O)—N(R₄)(R₅),—CH₂—NH—S(═O)₂(cyclopropyl),—CH₂—NH—C(═O) CH₃,—CH₂—O—R₁₆,

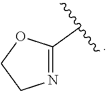 [SMILES: *C1=NCCO1]

—CH₂—OH, —CN, and
R₂ can be selected from —C(═O)—N(Ra) (R₅),—CH₂—NH—S(═O)> (cyclopropyl),—CH₂—O—CH₃,—CH₂—OH, and —CN.
R₂ can be selected from H, —C(═O)—N(R₄)(R₅),—CH₂—NH—C(═O) CH₃,—CH₂—NH—S(═O)₂R₃₅,—CH₂—OH, —CH₂—O—R₁₆,

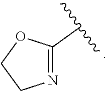 [SMILES: *C1=NCCO1]

and
R₂ can be selected from —C(═O)—N(R₄)(R₅),—CH₂—NH—C(═O) CH₃,—CH₂—NH—S(═O)₂R₃s,—CH₂—OH, —CH₂—O—R₁₆, and

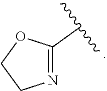 [SMILES: *C1=NCCO1]

R₂ can be selected from —C(═O)—N(R₄)(R₅),—CH₂—NH—C(═O) CH₃,—CH₂—NH—S(═O)₂(cyclopropyl),—CH₂—OH,

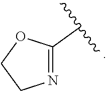 [SMILES: *C1=NCCO1]

—CH₂—O—CH₃, and
R₂ can be selected from —C(═O)—N(R₄)(R₅),—CH₂—NH—S(═O)₂(cyclopropyl),—CH₂—OH, and —CH₂—O—CH_{3.}
R₂ can be selected from H, —C(═O)—N(R₄)(R₅),—CH₂—NH—C(═O) CH₃,—CH₂—NH—S(═O)₂R₃₅,—CH₂—OH, and

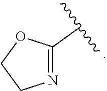 [SMILES: *C1=NCCO1]

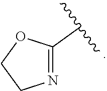 [SMILES: *C1=NCCO1]

R₂ can be selected from —C(═O)—N(R₄)(R₅),—CH₂—NH—C(═O) CH₃,—CH₂—NH—S(═O)₂R₃s,—CH₂—OH, and
R₂ can be selected from —C(═O)—N(R₄)(R₅),—CH₂—NH—C(═O) CH₃,—CH₂—NH—S(═O)₂(cyclopropyl),—CH₂—OH, and

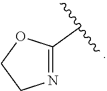 [SMILES: *C1=NCCO1]

R₂ can be selected from —C(═O)—N(R₄)(R₅),—CH₂—NH—S(═O)₂(cyclopropyl), and —CH₂—OH.
R₂ can be selected from —(CH₂)₀₋₃—C(═O)—N(R₄)(R₅),—CH₂—NH—S(═O)₂R₃₅, —C(═O)—O—R₆,—CH₂—O—CH₃, and —CN.
R₂ can be selected from —(CH₂)₀₋₃—C(═O)—N(R₄)(R₅), —C(═O)—O—R₆,—CH₂—O—CH₃, and —CN. R₂ can be selected from —(CH₂)₀₋₃—C(═O)—N(R₄)(R₅),—CH₂—NH—S(═O)₂R₃s, —C(═O)—O—R₆, and —CH₂—O—CH_{3.}
R₂ can be selected from —(CH₂)₀₋₃—C(═O)—N(R₄)(R₅), —C(═O)—O—R₆, and —CH₂—O—CH_{3.}
R₂ can be selected from —(CH₂) o-3—C(═O)—N(R₄)(R₅),—CH₂—O—CH₃, and —CN.
R₂ can be selected from —(CH₂)₀₋₃—C(═O)—N(R₄)(R₅), and —CH₂—O—CH_{3.}

Preferably, Rz is selected from —C(═O)—N(R₄)(R₅),—CH₂—NH—S(═O)₂R₃₅, —C(═O)—O—R₆,—CH₂—O—CH₃, and —CN.
R₂ can be selected from —C(═O)—N(R₄)(R₅), —C(═O)—O—R₆,—CH₂—O—CH₃, and —CN.
R₂ can be selected from —C(═O)—N(R₄)(R₅), —C(═O)—O—R₆, and —CH₂—O—CH_{3.}
R₂ can be selected from —C(═O)—N(R₄)(R₅),—CH₂—O—CH₃, and —CN.
R₂ can be selected from —C(═O)—N(R₄)(R₅), and —CH₂—O—CH_{3.}

More preferably, R₂ is-C(═O)—N(Ra) (R₅).

Preferably, R₄ is H or small alkyl.
R₄ can be H or CH_{3.}
R₄ can be H.
R₅ can be selected from alkyl, —OH, —S(═O)₂-(small alkyl), —(CH₂) 1-2—(C═O)—OH, —(CH₂)₀₋₂-cycloalkyl^{b}, —(CH₂)₀₋₂-heterocycloalkyl, —(CH(alkyl))-heterocycloalkyl, —(CH₂)₀₋₂-aryl, —(CH₂) o-2-polycycloalkyl, —(C(H)(phenyl))-alkyl, —(CH₂)₀₋₂heteroaryl^{a}, —(C(H)(heteroaryl))-alkyl, —(C(H)(alkyl))—(C═O)—OH, and —(C(H)(heteroaryl))—(C═O)—OR_{28.}
R₅ can be selected from alkyl, —OH, —S(═O)₂-(small alkyl), —(CH₂) 1-2—(C═O)—OH, —(CH₂)₀₋₂-cycloalkyl^{a}, —(CH₂)₀₋₂-heterocycloalkyl, —(CH(alkyl))-heterocycloalkyl, —(CH₂)₀₋₂-aryl, —(CH₂)₀₋₂-polycycloalkyl, —(C(H)(phenyl))-alkyl, —(CH₂)₀₋₂heteroaryl^{a}, —(C(H)(heteroaryl))-alkyl, —(C(H)(alkyl))—(C═O)—OH, and —(C(H)(heteroaryl))—(C═O)—OR_{28.}
R₅ can be selected from alkyl, —(CH₂)₀₋₂-cycloalkyl, —(CH₂)₀₋₂-heterocycloalkyl, —(CH(alkyl))-heterocycloalkyl, —(CH₂)₀₋₂-aryl, —(CH₂)₀₋₂-polycycloalkyl, —(C(H)(phenyl))-alkyl, —(CH₂) o-2heteroaryl^{a}, and —(C(H)(heteroaryl))-alkyl.

R₅ can be selected from alkyl, —(CH₂)₀₋₂-cycloalkyl, —(CH₂)₀₋₂-heterocycloalkyl, —(CH₂)₀₋₂-aryl, —(CH₂) o-2-polycycloalkyl, —(C(H)(phenyl))-alkyl, —(CH₂) o-2heteroaryl^{a}, and —(C(H)(heteroaryl))-alkyl.
R₅ can be selected from alkyl, —OH, —S(═O)₂-(small alkyl), —(CH₂) 1-2—(C═O)—OH, —(CH₂)₀₋₂-cycloalkyl^{b}, —(CH₂)₀₋₂-heterocycloalkyl, —(CH(alkyl))-heterocycloalkyl, —(CH₂)₀₋₂-aryl, —(CH₂)₀₋₂-polycycloalkyl, —(C(H)(phenyl))-alkyl, —(CH₂)₀₋₂heteroaryl, —(C(H)(heteroaryl))-alkyl, —(C(H)(alkyl))—(C═O)—OH, and —(C(H)(heteroaryl))—(C═O)—OR_{28.}
R₅ can be selected from alkyl, —OH, —S(═O)₂-(small alkyl), —(CH₂) 1-2—(C═O)—OH, —(CH₂)₀₋₂-cycloalkyl^{a}, —(CH₂)₀₋₂-heterocycloalkyl, —(CH(alkyl))-heterocycloalkyl, —(CH₂)₀₋₂-aryl, —(CH₂)₀₋₂-polycycloalkyl, —(C(H)(phenyl))-alkyl, —(CH₂)₀₋₂heteroaryl, —(C(H)(heteroaryl))-alkyl, —(C(H)(alkyl))—(C═O)—OH, and —(C(H)(heteroaryl))—(C═O)—OR_{28.}
R₅ can be selected from alkyl, —(CH₂)₀₋₂-cycloalkyl, —(CH₂)₀₋₂-heterocycloalkyl, —(CH(alkyl))-heterocycloalkyl, —(CH₂)₀₋₂-aryl, —(CH₂)₀₋₂-polycycloalkyl, —(C(H)(phenyl))-alkyl, —(CH₂)₀₋₂heteroaryl, and —(C(H)(heteroaryl))-alkyl.

R₅ can be selected from alkyl, —(CH₂)₀₋₂-cycloalkyl, —(CH₂)₀₋₂-heterocycloalkyl, —(CH₂)₀₋₂-aryl, —(CH₂)₀₋₂-polycycloalkyl, —(C(H)(phenyl))-alkyl, —(CH₂)₀₋₂heteroaryl, and —(C(H)(heteroaryl))-alkyl.

Preferably, R₅ is selected from alkyl, —OH, —S(═O)₂-(small alkyl), —(CH₂) 1-2—(C═O)—OH, —(CH₂)₀₋₂-cycloalkyl^{b}, —(CH₂)₀₋₂-heterocycloalkyl, —(CH(alkyl))-heterocycloalkyl, —(CH₂)₀₋₂-polycycloalkyl, —(CH₂)₀₋₂-phenyl, —(C(H)(phenyl))-alkyl, —(CH₂)₀₋₂heteroaryl, —(C(H)(heteroaryl))-alkyl, —(C(H)(alkyl))—(C═O)—OH, and —(C(H)(heteroaryl))—(C═O)—OR_{28.}
R₅ can be selected from alkyl, —OH, —S(═O)₂-(small alkyl), —(CH₂) 1-2—(C═O)—OH, —(CH₂)₀₋₂-cycloalkyl^{a}, —(CH₂)₀₋₂-heterocycloalkyl, —(CH(alkyl))-heterocycloalkyl, —(CH₂)₀₋₂-polycycloalkyl, —(CH₂)₀₋₂-phenyl, —(C(H)(phenyl))-alkyl, —(CH₂)₀₋₂heteroaryl, —(C(H)(heteroaryl))-alkyl, —(C(H)(alkyl))—(C═O)—OH, and —(C(H)(heteroaryl))—(C═O)—OR_{28.}
R₅ can from alkyl, —(CH₂)₀₋₂-cycloalkyl, —(CH₂)₀₋₂-heterocycloalkyl, be selected —(CH(alkyl))-heterocycloalkyl, —(CH₂)₀₋₂-polycycloalkyl, —(CH₂)₀₋₂-phenyl, —(C(H)(phenyl))-alkyl, —(CH₂) o-2heteroaryl, and —(C(H)(heteroaryl))-alkyl.
R₅ can be selected from alkyl, —(CH₂)₀₋₂-cycloalkyl, —(CH₂)₀₋₂-heterocycloalkyl, —(CH₂)₀₋₂-phenyl, —(CH₂)₀₋₂-polycycloalkyl, —(C(H)(phenyl))-alkyl, —(CH₂)₀₋₂heteroaryl, and —(C(H)(heteroaryl))-alkyl.

R₅ can be selected from-OH, —S(═O)₂-(small alkyl), —(CH₂) 1-2—(C═O)—OH, —(CH₂)₀₋₂-cycloalkyl^{a}, —(CH(alkyl))-heterocycloalkyl, —(C(H)(phenyl))-alkyl, —(CH₂) o-zheteroaryl, —(C(H)(alkyl))—(C═O)—OH, and —(C(H)(heteroaryl))—(C═O)—OR_{28.}
R₅ can be selected from —(CH₂)₀₋₂-cycloalkyl, —(CH(alkyl))-heterocycloalkyl, —(C(H)(phenyl))-alkyl, and —(CH₂) o-2heteroaryl.
R₅ can be selected from —(CH₂)₀₋₂-cycloalkyl^{b}, —(C(H)(phenyl))-alkyl, and —(CH₂)₀₋₂heteroaryl.
R₅ can be selected from —(CH₂)₀₋₂-cycloalkyl^{a}, —(C(H)(phenyl))-alkyl, and —(CH₂) o-zheteroaryl.

R₅ can be selected from —(CH₂)₀₋₂-cycloalkyl, —(C(H)(phenyl))-alkyl, and —(CH₂)₀₋₂heteroaryl.

R₅ can be —(CH₂) o-2heteroaryl.

R₅ can be selected from alkyl, —OH, —S(═O)₂-(small alkyl), —(CH₂)₁₋₃—(C═O)—OR₃₃, —(CH₂)₁₋₃—(C═O)—N(R₃₆)(R₃₇), —(CH₂)₀₋₃-cycloalkyl^{b}, —(CH₂)₀₋₃-heterocycloalkyl, —(C(H)(alkyl))—(C═O)—OR₃₈, —(CH(alkyl))-heterocycloalkyl, —(CH₂)₀₋₃-polycycloalkyl, and —(C(H)(alkyl))—(C═O)—N(R₃₉)(R₄₀).

R₅ can be selected from alkyl, —(CH₂)₀₋₃-cycloalkyl^{b}, —(CH₂)₀₋₃-heterocycloalkyl, —(CH(alkyl)-heterocycloalkyl, and —(CH₂)₀₋₃-polycycloalkyl.
R₅ can be selected from alkyl, —(CH₂)₀₋₃-cyclopropyl, —(CH₂)₀₋₃-heterocycloalkyl, —(CH(alkyl))-heterocycloalkyl, and —(CH₂)₀₋₃-polycycloalkyl.

R₅ can be selected from alkyl, —(CH₂)₀₋₃-cyclopropyl, and —(CH₂)₀₋₃-heterocycloalkyl.

R₅ can be selected from alkyl and —(CH₂)₀₋₃-heterocycloalkyl.
R₅ can be selected from alkyl and heterocycloalkyl.

Preferably, R₅ is selected from small alkyl, pyrrolidine, piperidine, and tetrahydrofuran, wherein the pyrrolidine, piperidine, and tetrahydrofuran may optionally be substituted with 1 or 2 substituents independently selected from small alkyl, cyclopropyl, small alkoxy, OH, CN, CF₃, and halo.
R₆ can be selected from H, small alkyl, and —(CH₂)-phenyl.
R₁₄ can be selected from H and small alkyl.
R₁₅ can be selected from H, alkyl, —(CH₂)₀₋₃-cycloalkyl, —(CH₂)₀₋₃-heterocycloalkyl, —(CH(alkyl))-heterocycloalkyl, —(CH₂)₀₋₃-aryl, —(C(H)(phenyl))-alkyl, —(CH₂)₀₋₃heteroaryl, and —(C(H)(heteroaryl))-alkyl.
R₁₅ can be selected from H, alkyl, —(CH₂)₀₋₃-cycloalkyl, —(CH₂)₀₋₃-heterocycloalkyl, —(CH₂)₀₋₃-aryl, —(C(H)(phenyl))-alkyl, —(CH₂)₀₋₃heteroaryl, and —(C(H)(heteroaryl))-alkyl.
R₁₅ can be selected from H and small alkyl.
R₁₅ can be methyl.
R₁₆ can be selected from H, small alkyl, cycloalkyl, —(CH₂)—(C═O)—OH, heterocycloalkyl, and —(CH₂)-heteroaryl.
R₁₆ can be selected from H, small alkyl, —(CH₂)—(C═O)—OH, heterocycloalkyl, and —(CH₂)-heteroaryl.
R₃₄ can be selected from H and small alkyl.
R₃₄ can be selected from H and CH_{3.}
R₃₅ can be selected from H, alkyl, —(CH₂)₀₋₂-cycloalkyl, —(CH₂)₀₋₂-heterocycloalkyl, —(CH(alkyl))-heterocycloalkyl, —(CH₂)₀₋₂-polycycloalkyl, —(CH₂)₀₋₂-phenyl, —(C(H)(phenyl))-alkyl, —(CH₂)₀₋₂heteroaryl, and —(C(H)(heteroaryl))-alkyl.
R₃₅ can be selected from H, alkyl, and cycloalkyl.
R₃₅ can be selected from H, small alkyl, and cyclopropyl.
R₄ can be H or small alkyl, and R₅ can be selected from alkyl, —OH, —S(═O)₂-(small alkyl), —(CH₂)₁₋₃—(C═O)—OH, —(CH₂)₀₋₃-cycloalkyl^{b}, —(CH₂)₀₋₃-heterocycloalkyl, —(CH(alkyl))-heterocycloalkyl, —(CH₂)₀₋₃-polycycloalkyl, —(CH₂)₀₋₃-aryl, —(C(H)(phenyl))-alkyl, —(CH₂)₀₋₃heteroaryl^{a}, —(C(H)(heteroaryl))-alkyl, —(C(H)(alkyl))—(C═O)—OH, and —(C(H)(heteroaryl))—(C═O)—OR_{28;}
or R₄ and R₅ together with the nitrogen atom to which they are attached can form a carbon-containing 4-, 5-, or 6-membered saturated heterocyclic ring, optionally containing an additional heteroatom selected from N, and O, which may be optionally mono- or di-substituted with substituents selected from oxo, alkyl, alkoxy, OH, halo and CF₃; and
R₆, where present, can be selected from H, alkyl, —(CH₂)₀₋₃-cycloalkyl, —(CH₂)₀₋₃-heterocycloalkyl, —(CH(alkyl))-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, and —(C(H)(heteroaryl))-alkyl.
R₄ can be H or small alkyl, and

R₅ can be selected from alkyl, —OH, —S(═O)₂-(small alkyl), —(CH₂)₁₋₃—(C═O)—OH, —(CH₂)₀₋₃-cycloalkyl^{a}, —(CH₂)₀₋₃-heterocycloalkyl, —(CH(alkyl))-heterocycloalkyl, —(CH₂)₀₋₃-polycycloalkyl, —(CH₂)₀₋₃-aryl, —(C(H)(phenyl))-alkyl, —(CH₂)₀₋₃heteroaryl^{a}, —(C(H)(heteroaryl))-alkyl, —(C(H)(alkyl))—(C═O)—OH, and —(C(H)(heteroaryl))—(C═O)—OR_{28;}
or R₄ and R₅ together with the nitrogen atom to which they are attached can form a carbon-containing 4-, 5-, or 6-membered saturated heterocyclic ring, optionally containing an additional heteroatom selected from N, and O, which may be optionally mono- or di-substituted with substituents selected from oxo, alkyl, alkoxy, OH, halo and CF₃; and
R₆, where present, can be selected from H, alkyl, —(CH₂)₀₋₃-cycloalkyl, —(CH₂)₀₋₃-heterocycloalkyl, —(CH(alkyl))-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, and —(C(H)(heteroaryl))-alkyl.
R₄ can be H or small alkyl, and
R₅ can be selected from alkyl, —(CH₂)₀₋₃-cycloalkyl, —(CH₂)₀₋₃-heterocycloalkyl, —(CH(alkyl))-heterocycloalkyl, —(CH₂)₀₋₃-polycycloalkyl, —(CH₂)₀₋₃-aryl, —(C(H)(phenyl))-alkyl, —(CH₂)₀₋₃heteroaryl^{a}, and —(C(H)(heteroaryl))-alkyl;
or R₄ and R₅ together with the nitrogen atom to which they are attached can form a carbon-containing 5-, or 6-membered saturated heterocyclic ring, optionally containing an additional heteroatom selected from N, and O, which may be optionally mono- or di-substituted with substituents selected from oxo, alkyl, alkoxy, OH, halo and CF₃; and
R₆, where present, can be selected from H, alkyl, —(CH₂)₀₋₃-cycloalkyl, —(CH₂)₀₋₃-heterocycloalkyl, —(CH(alkyl))-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, and —(C(H)(heteroaryl))-alkyl.
R₄ can be H or small alkyl, and
R₅ can be selected from alkyl, —(CH₂)₀₋₃-cycloalkyl, —(CH₂)₀₋₃-heterocycloalkyl, —(CH₂)₀₋₃-polycycloalkyl, —(CH₂)₀₋₃-aryl, —(C(H)(phenyl))-alkyl, —(CH₂)₀₋₃heteroaryl^{a}, and —(C(H)(heteroaryl))-alkyl;
or R₄ and R₅ together with the nitrogen atom to which they are attached can form a carbon-containing 5-, or 6-membered saturated heterocyclic ring, optionally containing an additional heteroatom selected from N, and O, which may be optionally mono- or di-substituted with substituents selected from oxo, alkyl, alkoxy, OH, halo and CF₃; and R₆, where present, can be selected from H, alkyl, —(CH₂)₀₋₃-cycloalkyl, —(CH₂)₀₋₃-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, and —(C(H)(heteroaryl))-alkyl.
R₄ can be H or small alkyl, and

R₅ can be selected from alkyl, —OH, —S(═O)₂-(small alkyl), —(CH₂)₁₋₃—(C═O)—OH, —(CH₂)₀₋₃-cycloalkyl^{b}, —(CH₂)₀₋₃-heterocycloalkyl, —(CH(alkyl))-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, —(C(H)(heteroaryl))-alkyl, —(C(H)(alkyl))—(C═O)—OH, and —(C(H)(heteroaryl))—(C═O)—OR_{28;}
or R₄ and R₅ together with the nitrogen atom to which they are attached can form a carbon-containing 4-, 5-, or 6-membered saturated heterocyclic ring, optionally containing an additional heteroatom selected from N, and O, which may be optionally mono- or di-substituted with substituents selected from oxo, alkyl, alkoxy, OH, halo and CF₃; and
R₆, where present, can be selected from H, alkyl, —(CH₂)₀₋₃-cycloalkyl, —(CH₂)₀₋₃-heterocycloalkyl, —(CH(alkyl))-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, and —(C(H)(heteroaryl))-alkyl.
R₄ can be H or small alkyl, and

R₅ can be selected from alkyl, —OH, —S(═O)₂-(small alkyl), —(CH₂)₁₋₃—(C═O)—OH, —(CH₂)₀₋₃-cycloalkyl^{a}, —(CH₂)₀₋₃-heterocycloalkyl, —(CH(alkyl))-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, —(C(H)(heteroaryl))-alkyl, —(C(H)(alkyl))—(C═O)—OH, and —(C(H)(heteroaryl))—(C═O)—OR_{28;}
or R₄ and R₅ together with the nitrogen atom to which they are attached can form a carbon-containing 4-, 5-, or 6-membered saturated heterocyclic ring, optionally containing an additional heteroatom selected from N, and O, which may be optionally mono- or di-substituted with substituents selected from oxo, alkyl, alkoxy, OH, halo and CF₃; and
R₆, where present, can be selected from H, alkyl, —(CH₂)₀₋₃-cycloalkyl, —(CH₂)₀₋₃-heterocycloalkyl, —(CH(alkyl))-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, and —(C(H)(heteroaryl))-alkyl.
R₄ can be H or small alkyl, and

R₅ can be selected from alkyl, —(CH₂)₀₋₃-cycloalkyl, —(CH₂)₀₋₃-heterocycloalkyl, —(CH(alkyl))-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, and —(C(H)(heteroaryl))-alkyl;
or R₄ and R₅ together with the nitrogen atom to which they are attached can form a carbon-containing 5-, or 6-membered saturated heterocyclic ring, optionally containing an additional heteroatom selected from N, and O, which may be optionally mono- or di-substituted with substituents selected from oxo, alkyl, alkoxy, OH, halo and CF₃; and
R₆, where present, can be selected from H, alkyl, —(CH₂)₀₋₃-cycloalkyl, —(CH₂)₀₋₃-heterocycloalkyl, —(CH(alkyl))-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, and —(C(H)(heteroaryl))-alkyl.
R₄ can be H or small alkyl, and
R₅ can be selected from alkyl, —(CH₂)₀₋₃-cycloalkyl, —(CH₂)₀₋₃-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, and —(C(H)(heteroaryl))-alkyl;
or R₄ and R₅ together with the nitrogen atom to which they are attached can form a carbon-containing 5-, or 6-membered saturated heterocyclic ring, optionally containing an additional heteroatom selected from N, and O, which may be optionally mono- or di-substituted with substituents selected from oxo, alkyl, alkoxy, OH, halo and CF₃; and
R₆, where present, can be selected from H, alkyl, —(CH₂)₀₋₃-cycloalkyl, —(CH₂)₀₋₃-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, and —(C(H)(heteroaryl))-alkyl.
R₄ can be H or small alkyl, and

R₅ can be selected from alkyl, —OH, —S(═O)₂-(small alkyl), —(CH₂)₁₋₃—(C═O)—OH, —(CH₂)₀₋₃-cycloalkyl^{b}, —(CH₂)₀₋₃-heterocycloalkyl, —(CH(alkyl))-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, —(C(H)(heteroaryl))-alkyl, —(C(H)(alkyl))—(C═O)—OH, and —(C(H)(heteroaryl))—(C═O)—OR_{28;}
or R₄ and R₅ together with the nitrogen atom to which they are attached can form a carbon-containing 4-, 5-, or 6-membered saturated heterocyclic ring, optionally containing an additional heteroatom selected from N, and O, which may be optionally mono- or di-substituted with substituents selected from oxo, alkyl, alkoxy, OH, halo and CF₃; and
R₆, where present, can be H, small alkyl, and —(CH₂)-phenyl.
R₄ can be H or small alkyl, and
R₅ can be selected from alkyl, —OH, —S(═O)₂-(small alkyl), —(CH₂)₁₋₃—(C═O)—OH, —(CH₂)₀₋₃-cycloalkyl^{a}, —(CH₂)₀₋₃-heterocycloalkyl, —(CH(alkyl))-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, —(C(H)(heteroaryl))-alkyl, —(C(H)(alkyl))—(C═O)—OH, and —(C(H)(heteroaryl))—(C═O)—OR_{28;}
or R₄ and R₅ together with the nitrogen atom to which they are attached can form a carbon-containing 4-, from N, and O, which may be optionally mono- or di-substituted with substituents selected from oxo, alkyl, alkoxy, OH, halo and CF₃; and R₆, where present, can be H, small alkyl, and —(CH₂)-phenyl.
R₄ can be H or small alkyl, and
R₅ can be selected from alkyl, —(CH₂)₀₋₃-cycloalkyl, —(CH₂)₀₋₃-heterocycloalkyl, —(CH(alkyl))-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, and —(C(H)(heteroaryl))-alkyl;
or R₄ and R₅ together with the nitrogen atom to which they are attached can form a carbon-containing 5-, or 6-membered saturated heterocyclic ring, optionally containing an additional heteroatom selected from N, and O, which may be optionally mono- or di-substituted with substituents selected from oxo, alkyl, alkoxy, OH, halo and CF₃; and R₆, where present, can be H, small alkyl, and —(CH₂)-phenyl.
R₄ can be H or small alkyl, and
R₅ can be selected from alkyl, —(CH₂)₀₋₃-cycloalkyl, —(CH₂)₀₋₃-heterocycloalkyl, —(CH₂)₀₋₃-polycycloalkyl, —(CH₂)₀₋₃-aryl, —(C(H)(phenyl)-alkyl, —(CH₂)₀₋₃heteroaryl, and —(C(H)(heteroaryl)-alkyl;
or R₄ and R₅ together with the nitrogen atom to which they are attached can form a carbon-containing 5-, or 6-membered saturated heterocyclic ring, optionally containing an additional heteroatom selected from N, and O, which may be optionally mono- or di-substituted with substituents selected from oxo, alkyl, alkoxy, OH, halo and CF₃; and R₆, where present, can be H, small alkyl, and —(CH₂)-phenyl.
R₄ can be H or CH₃, and

R₅ can be selected from alkyl, —OH, —S(═O)₂-(small alkyl), —(CH₂)₁₋₃—(C═O)—OH, —(CH₂)₀₋₃-cycloalkyl^{b}, —(CH₂)₀₋₃-heterocycloalkyl, —(CH(alkyl))-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, —(C(H)(heteroaryl))-alkyl, —(C(H)(alkyl))—(C═O)—OH, and —(C(H)(heteroaryl))—(C═O)—OR_{28;}
or R₄ and R₅ together with the nitrogen atom to which they are attached can form a carbon-containing 4-, from N, and O, which may be optionally mono- or di-substituted with substituents selected from oxo, alkyl, alkoxy, OH, halo and CF₃; and R₆, where present, can be H, small alkyl, and —(CH₂)-phenyl.
R₄ can be H or CH₃, and

R₅ can be selected from alkyl, —OH, —S(═O)₂-(small alkyl), —(CH₂)₁₋₃—(C═O)—OH, —(CH₂)₀₋₃-cycloalkyl^{a}, —(CH₂)₀₋₃-heterocycloalkyl, —(CH(alkyl))-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, —(C(H)(heteroaryl))-alkyl, —(C(H)(alkyl))—(C═O)—OH, and —(C(H)(heteroaryl))—(C═O)—OR_{28;}
or R₄ and R₅ together with the nitrogen atom to which they are attached can form a carbon-containing 4-, 5-, or 6-membered saturated heterocyclic ring, optionally containing an additional heteroatom selected from N, and O, which may be optionally mono- or di-substituted with substituents selected from oxo, alkyl, alkoxy, OH, halo and CF₃; and
R₆, where present, can be H, small alkyl, and —(CH₂)-phenyl.
R₄ can be H or CH₃, and

R₅ can be selected from alkyl, —(CH₂)₀₋₃-cycloalkyl, —(CH₂)₀₋₃-heterocycloalkyl, —(CH(alkyl))-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, and —(C(H)(heteroaryl))-alkyl;
or R₄ and R₅ together with the nitrogen atom to which they are attached can form a carbon-containing 5-, or 6-membered saturated heterocyclic ring, optionally containing an additional heteroatom selected from N, and O, which may be optionally mono- or di-substituted with substituents selected from oxo, alkyl, alkoxy, OH, halo and CF₃; and
R₆, where present, can be H, small alkyl, and —(CH₂)-phenyl.
R₄ can be H or CH₃, and

R₅ can be selected from alkyl, —(CH₂)₀₋₃-cycloalkyl, —(CH₂)₀₋₃-heterocycloalkyl, —(CH(alkyl))-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, and —(C(H)(heteroaryl))-alkyl;
or R₄ and R₅ together with the nitrogen atom to which they are attached can form a carbon-containing 5-, or 6-membered saturated heterocyclic ring, optionally containing an additional heteroatom selected from N, and O, which may be optionally mono- or di-substituted with substituents selected from oxo, alkyl, alkoxy, OH, halo and CF₃; and
R₆, where present, can be H, small alkyl, and —(CH₂)-phenyl.
R₄ can be H or CH₃, and R₅ can be selected from alkyl, —(CH₂)₀₋₃-cycloalkyl^{b}, —(CH₂)₀₋₃-heterocycloalkyl, —(CH(alkyl))-heterocycloalkyl, and —(CH₂)₀₋₃-polycycloalkyl; and
R₆, where present, can be H, small alkyl, and —(CH₂)-phenyl.
R₄ can be H or CH₃, and

R₅ is selected from small alkyl, pyrrolidine, piperidine, and tetrahydrofuran, wherein the pyrrolidine, piperidine, and tetrahydrofuran may optionally be substituted with 1 or 2 substituents independently selected from small alkyl, cyclopropyl, small alkoxy, OH, CN, CF₃, and halo; and R₆, where present, can be H, small alkyl, and —(CH₂)-phenyl.
R₂ can be selected from —(CH₂)₀₋₃—C(═O)—N(R₄)(R₅), —(CH₂)₀₋₃—NR₁₄—C(═O) R₁₅, —(CH₂)₀₋₃—NR₃₄—S(═O)₂R₃₅, alkyl, cycloalkyl, heterocycloalkyl, alkoxy, —OH, —CF₃, halo, aryl, heteroaryl, —CH₂—O—R₁₆ and —CN, wherein:
R₄ is H or small alkyl, and

R₅ is selected from alkyl, OH, —S(═O)₂-(small alkyl), —(CH₂)₁₋₃—(C═O)—OH, —(CH₂)₀₋₃-cycloalkyl^{b}, —(CH₂)₀₋₃-heterocycloalkyl, —(CH(alkyl))-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, —(C(H)(heteroaryl))-alkyl, —(C(H)(alkyl))—(C═O)—OH, and —(C(H)(heteroaryl))—(C═O)—OR_{28;}
or R₄ and R₅ together with the nitrogen atom to which they are attached can form a carbon-containing 4-, 5-, or 6-membered saturated heterocyclic ring, optionally containing an additional heteroatom selected from N, and O, which may be optionally mono- or di-substituted with substituents selected from oxo, alkyl, alkoxy, OH, halo and CF₃; R₆ is H, small alkyl, and —(CH₂)-phenyl;
R₁₄ is selected from H and small alkyl; R₁₅ is selected from H and small alkyl; R₁₆ is selected from H, small alkyl, —(CH₂)—(C═O)—OH, heterocycloalkyl, and —(CH₂)-heteroaryl; R₃₄ is selected from H and small alkyl; and
R₃₅ is selected from H, small alkyl, and cyclopropyl.
R₂ can be selected from H, —(CH₂)₀₋₃—C(═O)—N(R₄)(R₅), —(CH₂)₀₋₃—NR₁₄—C(═O) R₁₅, —(CH₂)₀₋₃—NR₃₄—S(═O)₂R₃₅, alkyl, cycloalkyl, heterocycloalkyl, alkoxy, —OH, —CF₃, halo, aryl, heteroaryl, —CH₂—O—R₁₆ and —CN, wherein: R₄ is H or small alkyl, and R₅ is selected from alkyl, OH, —S(═O)₂-(small alkyl), —(CH₂)₁₋₃—(C═O)—OH, —(CH₂)₀₋₃-cycloalkyl^{a}, —(CH₂)₀₋₃-heterocycloalkyl, —(CH(alkyl))-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, —(C(H)(heteroaryl))-alkyl, —(C(H)(alkyl))—(C═O)—OH, and —(C(H)(heteroaryl))—(C═O)—OR_{28;}
or R₄ and R₅ together with the nitrogen atom to which they are attached can form a carbon-containing 4-, from N, and O, which may be optionally mono- or di-substituted with substituents selected from oxo, alkyl, alkoxy, OH, halo and CF₃; and R₆ is H, small alkyl, and —(CH₂)-phenyl.
R₂ can be selected from H, —(CH₂)₀₋₃—C(═O)—N(R₄)(R₅), —(CH₂)₀₋₃—NR₁₄—C(═O) R₁₅, —(CH₂)₀₋₃—NR₃₄—S(═O)₂R₃₅, alkyl, cycloalkyl, heterocycloalkyl, alkoxy, —OH, —CF₃, halo, aryl, heteroaryl, —CH₂—O—R₁₆ and —CN, wherein:
R₄ is H or small alkyl, and
R₅ is selected from alkyl, —(CH₂)₀₋₃-cycloalkyl, —(CH₂)₀₋₃-heterocycloalkyl, —(CH(alkyl))-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, and —(C(H)(heteroaryl))-alkyl;
or R₄ and R₅ together with the nitrogen atom to which they are attached can form a carbon-containing 5-, or 6-membered saturated heterocyclic ring, optionally containing an additional heteroatom selected from N, and O, which may be optionally mono- or di-substituted with substituents selected from oxo, alkyl, alkoxy, OH, halo and CF₃; and
R₆ is H, small alkyl, and —(CH₂)-phenyl.
R₂ can be selected from H, —(CH₂)₀₋₃—C(═O)—N(R₄)(R₅), —(CH₂)₀₋₃—NR₁₄—C(═O) R₁₅, alkyl, cycloalkyl, heterocycloalkyl, alkoxy, —OH, —CF₃, halo, aryl, heteroaryl, —CH₂—O—R₁₆ and —CN, wherein:
R₄ is H or small alkyl, and
R₅ is selected from alkyl, —(CH₂)₀₋₃-cycloalkyl, —(CH₂)₀₋₃-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, and —(C(H)(heteroaryl))-alkyl;
or R₄ and R₅ together with the nitrogen atom to which they are attached can form a carbon-containing 5-, or 6-membered saturated heterocyclic ring, optionally containing an additional heteroatom selected from N, and O, which may be optionally mono- or di-substituted with substituents selected from oxo, alkyl, alkoxy, OH, halo and CF₃; and R₆ is H, small alkyl, and —(CH₂)-phenyl.
R₂ can be selected from —(CH₂)₀₋₃—C(═O)—N(R₄)(R₅), —C(═O)—O—R₆, —(CH₂)₀₋₃—NR₁₄—C(═O) R₁₅, —(CH₂)₀₋₃—NR₃₄—S(═O)₂R₃₅, alkyl, cycloalkyl, heterocycloalkyl, alkoxy, —OH, —CF₃, halo, aryl, heteroaryl, and —CH₂—O—R₁₆, wherein: R₄ is H or small alkyl, and
R₅ is selected from alkyl, OH, —S(═O)₂-(small alkyl), —(CH₂)₁₋₃—(C═O)—OH, —(CH₂)₀₋₃-cycloalkyl^{b}, —(CH₂)₀₋₃-heterocycloalkyl, —(CH(alkyl))-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, —(C(H)(heteroaryl))-alkyl, —(C(H)(alkyl))—(C═O)—OH, and —(C(H)(heteroaryl))—(C═O)—OR_{28;}
or R₄ and R₅ together with the nitrogen atom to which they are attached can form a carbon-containing 4-, 5-, or 6-membered saturated heterocyclic ring, optionally containing an additional heteroatom selected from N, and O, which may be optionally mono- or di-substituted with substituents selected from oxo, alkyl, alkoxy, OH, halo and CF₃; R₆ is H, small alkyl, and —(CH₂)-phenyl; R₁₄ is selected from H and small alkyl; R₁₅ is selected from H and small alkyl;
R₁₆ is selected from H, small alkyl, —(CH₂)—(C═O)—OH, heterocycloalkyl, and —(CH₂)-heteroaryl; R₃₄ is selected from H and small alkyl; and R₃₅ is selected from H, small alkyl, and cyclopropyl.
R₂ can be selected from H, —(CH₂)₀₋₃—C(═O)—N(R₄)(R₅), —C(═O)—O—R₆, —(CH₂)₀₋₃—NR₁₄—C(═O) R₁₅, —(CH₂)₀₋₃—NR₃₄—S(═O)₂R₃₅, alkyl, cycloalkyl, heterocycloalkyl, alkoxy, —OH, —CF₃, halo, aryl, heteroaryl, and —CH₂—O—R₁₆, wherein:
R₄ is H or small alkyl, and

R₅ is selected from alkyl, OH, —S(═O)₂-(small alkyl), —(CH₂)₁₋₃—(C═O)—OH, —(CH₂)₀₋₃-cycloalkyl^{a}, —(CH₂)₀₋₃-heterocycloalkyl, —(CH(alkyl))-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, —(C(H)(heteroaryl))-alkyl, —(C(H)(alkyl))—(C═O)—OH, and —(C(H)(heteroaryl))—(C═O)—OR_{28;}

or R₄ and R₅ together with the nitrogen atom to which they are attached can form a carbon-containing 4-, 5-, or 6-membered saturated heterocyclic ring, optionally containing an additional heteroatom selected from N, and O, which may be optionally mono- or di-substituted with substituents selected from oxo, alkyl, alkoxy, OH, halo and CF₃; and R₆ is H, small alkyl, and —(CH₂)-phenyl.
R₂ can be selected from H, —(CH₂)₀₋₃—C(═O)—N(R₄)(R₅), —C(═O)—O—R₆, —(CH₂)₀₋₃—NR₁₄—C(═O) R₁₅, —(CH₂)₀₋₃—NR₃₄—S(═O)₂R₃₅, alkyl, cycloalkyl, heterocycloalkyl, alkoxy, —OH, —CF₃, halo, aryl, heteroaryl, and —CH₂—O—R₁₆, wherein: R₄ is H or small alkyl, and R₅ is selected from alkyl, —(CH₂)₀₋₃-cycloalkyl, —(CH₂)₀₋₃-heterocycloalkyl, —(CH(alkyl)-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, and —(C(H)(heteroaryl))-alkyl;
or R₄ and R₅ together with the nitrogen atom to which they are attached can form a carbon-containing 5-, or 6-membered saturated heterocyclic ring, optionally containing an additional heteroatom selected from N, and O, which may be optionally mono- or di-substituted with substituents selected from oxo, alkyl, alkoxy, OH, halo and CF₃; and
R₂ is H, small alkyl, and —(CH₂)-phenyl.
R₂ can be selected from H, —(CH₂)₀₋₃—C(═O)—N(R₄)(R₅), —C(═O)—O—R₆, —(CH₂)₀₋₃—NR₁₄—C(═O) R₁₅, alkyl, cycloalkyl, heterocycloalkyl, alkoxy, —OH, —CF₃, halo, aryl, heteroaryl, and —CH₂—O—R₁₆, wherein:
R₄ is H or small alkyl, and
R₅ is selected from alkyl, —(CH₂)₀₋₃-cycloalkyl, —(CH₂)₀₋₃-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, and —(C(H)(heteroaryl))-alkyl;
or R₄ and R₅ together with the nitrogen atom to which they are attached can form a carbon-containing 5-, or 6-membered saturated heterocyclic ring, optionally containing an additional heteroatom selected from N, and O, which may be optionally mono- or di-substituted with substituents selected from oxo, alkyl, alkoxy, OH, halo and CF₃; and
R₆ is H, small alkyl, and —(CH₂)-phenyl.
R₂ can be selected from —(CH₂)₀₋₃—C(═O)—N(R₄)(R₅), —C(═O)—O—R₆, —(CH₂)₀₋₃—NR₁₄—C(═O) R₁₅, —(CH₂)₀₋₃—NR₃₄—S(═O)₂R₃₅, alkyl, cycloalkyl, heterocycloalkyl, alkoxy, —OH, halo, aryl, heteroaryl, —CH₂—O—R₁₆, and —CN, wherein: R₄ is H or small alkyl, and
R₅ is selected from alkyl, OH, —S(═O)₂-(small alkyl), —(CH₂)₁₋₃—(C═O)—OH, —(CH₂)₀₋₃-cycloalkyl^{b}, —(CH₂)₀₋₃-heterocycloalkyl, —(CH(alkyl))-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, —(C(H)(heteroaryl))-alkyl, —(C(H)(alkyl))—(C═O)—OH, and —(C(H)(heteroaryl))—(C═O)—OR_{28;}
or R₄ and R₅ together with the nitrogen atom to which they are attached can form a carbon-containing 4-, 5-, or 6-membered saturated heterocyclic ring, optionally containing an additional heteroatom selected from N, and O, which may be optionally mono- or di-substituted with substituents selected from oxo, alkyl, alkoxy, OH, halo and CF₃; R₆ is H, small alkyl, and —(CH₂)-phenyl; R₁₄ is selected from H and small alkyl; R₁₅ is selected from H and small alkyl;
R₁₆ is selected from H, small alkyl, —(CH₂)—(C═O)—OH, heterocycloalkyl, and —(CH₂)-heteroaryl;
R₃₄ is selected from H and small alkyl; and
R₃₅ is selected from H, small alkyl, and cyclopropyl.
R₂ can be selected from H, —(CH₂)₀₋₃—C(═O)—N(R₄)(R₅), —C(═O)—O—R₆, —(CH₂)₀₋₃—NR₁₄—C(═O) R₁₅, —(CH₂)₀₋₃—NR₃₄—S(═O)₂R₃₅, alkyl, cycloalkyl, heterocycloalkyl, alkoxy, —OH, halo, aryl, heteroaryl, —CH₂—O—R₁₆, and —CN, wherein:
R₄ is H or small alkyl, and

R₅ is selected from alkyl, OH, —S(═O)₂-(small alkyl), —(CH₂)₁₋₃—(C═O)—OH, —(CH₂)₀₋₃-cycloalkyl^{a}, —(CH₂)₀₋₃-heterocycloalkyl, —(CH(alkyl))-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, —(C(H)(heteroaryl))-alkyl, —(C(H)(alkyl))—(C═O)—OH, and —(C(H)(heteroaryl))—(C═O)—OR_{28;}
or R₄ and R₅ together with the nitrogen atom to which they are attached can form a carbon-containing 4-, 5-, or 6-membered saturated heterocyclic ring, optionally containing an additional heteroatom selected from N, and O, which may be optionally mono- or di-substituted with substituents selected from oxo, alkyl, alkoxy, OH, halo and CF₃; and
R₆ is H, small alkyl, and —(CH₂)-phenyl.
R₂ can be selected from H, —(CH₂)₀₋₃—C(═O)—N(R₄)(R₅), —C(═O)—O—R₆, —(CH₂)₀₋₃—NR₁₄—C(═O) R₁₅, —(CH₂)₀₋₃—NR₃₄—S(═O)₂R₃₅, alkyl, cycloalkyl, heterocycloalkyl, alkoxy, —OH, halo, aryl, heteroaryl, —CH₂—O—R₁₆, and —CN, wherein:
R₄ is H or small alkyl, and
R₅ is selected from alkyl, —(CH₂)₀₋₃-cycloalkyl, —(CH₂)₀₋₃-heterocycloalkyl, —(CH(alkyl))-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, and —(C(H)(heteroaryl))-alkyl;
or R₄ and R₅ together with the nitrogen atom to which they are attached can form a carbon-containing 5-, or 6-membered saturated heterocyclic ring, optionally containing an additional heteroatom selected from N, and O, which may be optionally mono- or di-substituted with substituents selected from oxo, alkyl, alkoxy, OH, halo and CF₃; and R₆ is H, small alkyl, and —(CH₂)-phenyl.
R₂ can be selected from H, —(CH₂)₀₋₃—C(═O)—N(R₄)(R₅), —C(═O)—O—R₆, —(CH₂)₀₋₃—NR₁₄—C(═O) R₁₅, alkyl, cycloalkyl, heterocycloalkyl, alkoxy, —OH, halo, aryl, heteroaryl, —CH₂—O—R₁₆, and —CN, wherein: R₄ is H or small alkyl, and is
R₅ alkyl, —(CH₂)₀₋₃-cycloalkyl, selected from —(CH₂)₀₋₃-heterocycloalkyl, —(CH₂)₀₋₃-polycycloalkyl, —(CH₂)₀₋₃-aryl, —(C(H)(phenyl)-alkyl, —(CH₂)₀₋₃heteroaryl, and —(C(H)(heteroaryl)-alkyl;
or R₄ and R₅ together with the nitrogen atom to which they are attached can form a carbon-containing 5-, or 6-membered saturated heterocyclic ring, optionally containing an additional heteroatom selected from N, and O, which may be optionally mono- or di-substituted with substituents selected from oxo, alkyl, alkoxy, OH, halo and CF₃; and
Ro is H, small alkyl, and —(CH₂)-phenyl.
R₂ can be selected from —(CH₂)₀₋₃—C(═O)—N(R₄)(R₅), —(CH₂)₀₋₃—NR₁₄—C(═O) R₁₅, —(CH₂)₀₋₃—NR₃₄—S(═O)₂R₃₅, alkyl, cycloalkyl, heterocycloalkyl, alkoxy, —OH, —CF₃, halo, aryl, heteroaryl, and —CH₂—O—R₁₆, wherein:
R₄ is H or small alkyl, and
R₅ is selected from alkyl, OH, —S(═O)₂-(small alkyl), —(CH₂)₁₋₃—(C═O)—OH, —(CH₂)₀₋₃-cycloalkyl^{b}, —(CH₂)₀₋₃-heterocycloalkyl, —(CH(alkyl))-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, —(C(H)(heteroaryl))-alkyl, —(C(H)(alkyl))—(C═O)—OH, and —(C(H)(heteroaryl))—(C═O)—OR_{28;}
or R₄ and R₅ together with the nitrogen atom to which they are attached can form a carbon-containing 4-, 5-, or 6-membered saturated heterocyclic ring, optionally containing an additional heteroatom selected from N, and O, which may be optionally mono- or di-substituted with substituents selected from oxo, alkyl, alkoxy, OH, halo and CF_{3;}
R₆ is H, small alkyl, and —(CH₂)-phenyl; R₁₄ is selected from H and small alkyl;
R₁₅ is selected from H and small alkyl; R₁₆ is selected from H, small alkyl, —(CH₂)—(C═O)—OH, heterocycloalkyl, and —(CH₂)-heteroaryl; R₃₄ is selected from H and small alkyl; and R₃₅ is selected from H, small alkyl, and cyclopropyl.
R₂ can be selected from H, —(CH₂)₀₋₃—C(═O)—N(R₄)(R₅), —(CH₂)₀₋₃—NR₁₄—C(═O) R₁₅, —(CH₂)₀₋₃—NR₃₄—S(═O)₂R₃₅, alkyl, cycloalkyl, heterocycloalkyl, alkoxy, —OH, —CF₃, halo, aryl, heteroaryl, and —CH₂—O—R₁₆, wherein: R₄ is H or small alkyl, and
R₅ is selected from alkyl, OH, —S(═O)₂-(small alkyl), —(CH₂)₁₋₃—(C═O)—OH, —(CH₂)₀₋₃-cycloalkyl^{a}, —(CH₂)₀₋₃-heterocycloalkyl, —(CH(alkyl))-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, —(C(H)(heteroaryl))-alkyl, —(C(H)(alkyl))—(C═O)—OH, and —(C(H)(heteroaryl))—(C═O)—OR_{28;}
or R₄ and R₅ together with the nitrogen atom to which they are attached can form a carbon-containing 4-, from N, and O, which may be optionally mono- or di-substituted with substituents selected from oxo, alkyl, alkoxy, OH, halo and CF_{3.}
R₂ can be selected from H, —(CH₂)₀₋₃—C(═O)—N(R₄)(R₅), —(CH₂)₀₋₃—NR₁₄—C(═O) R₁₅, —(CH₂)₀₋₃—NR₃₄—S(═O)₂R₃₅, alkyl, cycloalkyl, heterocycloalkyl, alkoxy, —OH, —CF₃, halo, aryl, heteroaryl, and —CH₂—O—R₁₆, wherein:
R₄ is H or small alkyl, and
R₅ is selected from alkyl, —(CH₂)₀₋₃-cycloalkyl, —(CH₂)₀₋₃-heterocycloalkyl, —(CH(alkyl))-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, and —(C(H)(heteroaryl))-alkyl;
or R₄ and R₅ together with the nitrogen atom to which they are attached can form a carbon-containing 5-, or 6-membered saturated heterocyclic ring, optionally containing an additional heteroatom selected from N, and O, which may be optionally mono- or di-substituted with substituents selected from oxo, alkyl, alkoxy, OH, halo and CF_{3.}
R₂ can be selected from H, —(CH₂)₀₋₃—C(═O)—N(R₄)(R₅), —(CH₂)₀₋₃—NR₁₄—C(═O) R₁₅, alkyl, cycloalkyl, heterocycloalkyl, alkoxy, —OH, —CF₃, halo, aryl, heteroaryl, and —CH₂—O—R₁₆, wherein:
R₄ is H or small alkyl, and
R₅ is selected from alkyl, —(CH₂)₀₋₃-cycloalkyl, —(CH₂)₀₋₃-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, and —(C(H)(heteroaryl))-alkyl;
or R₄ and R₅ together with the nitrogen atom to which they are attached can form a carbon-containing 5-, or 6-membered saturated heterocyclic ring, optionally containing an additional heteroatom selected from N, and O, which may be optionally mono- or di-substituted with substituents selected from oxo, alkyl, alkoxy, OH, halo and CF_{3.}
R₂ can be selected from —(CH₂)₀₋₃—C(═O)—N(R₄)(R₅), —(CH₂)₀₋₃—NR₁₄—C(═O) R₁₅, —(CH₂)₀₋₃—NR₃₄—S(═O)₂R₃₅, alkyl, cycloalkyl, heterocycloalkyl, alkoxy, —OH, halo, aryl, heteroaryl, —CH₂—O—R₁₆, and —CN, wherein: R₄ is H or small alkyl, and
R₅ is selected from alkyl, OH, —S(═O)₂-(small alkyl), —(CH₂)₁₋₃—(C═O)—OH, —(CH₂)₀₋₃-cycloalkyl^{b}, —(CH₂)₀₋₃-heterocycloalkyl, —(CH(alkyl))-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, —(C(H)(heteroaryl))-alkyl, —(C(H)(alkyl))—(C═O)—OH, and —(C(H)(heteroaryl))—(C═O)—OR_{28;}
or R₄ and R₅ together with the nitrogen atom to which they are attached can form a carbon-containing 4-, from N, and O, which may be optionally mono- or di-substituted with substituents selected from oxo, alkyl, alkoxy, OH, halo and CF_{3;}
R₆ is H, small alkyl, and —(CH₂)-phenyl;
R₁₄ is selected from H and small alkyl; R₁₅ is selected from H and small alkyl; R₁₆ is selected from H, small alkyl, —(CH₂)—(C═O)—OH, heterocycloalkyl, and —(CH₂)-heteroaryl; R₃₄ is selected from H and small alkyl; and
R₃₅ is selected from H, small alkyl, and cyclopropyl.
R₂ can be selected from H, —(CH₂)₀₋₃—C(═O)—N(R₄)(R₅), —(CH₂)₀₋₃—NR₁₄—C(═O) R₁₅, —(CH₂)₀₋₃—NR₃₄—S(═O)₂R₃₅, alkyl, cycloalkyl, heterocycloalkyl, alkoxy, —OH, halo, aryl, heteroaryl, —CH₂—O—R₁₆, and —CN, wherein:
R₄ is H or small alkyl, and
R₅ is selected from alkyl, OH, —S(═O)₂-(small alkyl), —(CH₂)₁₋₃—(C═O)—OH, —(CH₂)₀₋₃-cycloalkyl^{a}, —(CH₂)₀₋₃-heterocycloalkyl, —(CH(alkyl))-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, —(C(H)(heteroaryl))-alkyl, —(C(H)(alkyl))—(C═O)—OH, and —(C(H)(heteroaryl))—(C═O)—OR_{28;}
or R₄ and R₅ together with the nitrogen atom to which they are attached can form a carbon-containing 4-, 5-, or 6-membered saturated heterocyclic ring, optionally containing an additional heteroatom selected from N, and O, which may be optionally mono- or di-substituted with substituents selected from oxo, alkyl, alkoxy, OH, halo and CF_{3.}
R₂ can be selected from H, —(CH₂)₀₋₃—C(═O)—N(R₄)(R₅), —(CH₂)₀₋₃—NR₁₄—C(═O) R₁₅, —(CH₂)₀₋₃—NR₃₄—S(═O)₂R₃₅, alkyl, cycloalkyl, heterocycloalkyl, alkoxy, —OH, halo, aryl, heteroaryl, —CH₂—O—R₁₆, and —CN, wherein: R₄ is H or small alkyl, and

R₅ is selected from alkyl, —(CH₂)₀₋₃-cycloalkyl, —(CH₂)₀₋₃-heterocycloalkyl, —(CH(alkyl)-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, and —(C(H)(heteroaryl))-alkyl;
or R₄ and R₅ together with the nitrogen atom to which they are attached can form a carbon-containing 5-, or 6-membered saturated heterocyclic ring, optionally containing an additional heteroatom selected from N, and O, which may be optionally mono- or di-substituted with substituents selected from oxo, alkyl, alkoxy, OH, halo and CF_{3.}
R₂ can be selected from H, —(CH₂)₀₋₃—C(═O)—N(R₄)(R₅), —(CH₂)₀₋₃—NR₁₄—C(═O) R₁₅, alkyl, cycloalkyl, heterocycloalkyl, alkoxy, —OH, halo, aryl, heteroaryl, —CH₂—O—R₁₆, and —CN, wherein: R₄ is H or small alkyl, and is
R₅ alkyl, —(CH₂)₀₋₃-cycloalkyl, selected from —(CH₂)₀₋₃-heterocycloalkyl, —(CH₂)₀₋₃-polycycloalkyl, —(CH₂)₀₋₃-aryl, —(C(H)(phenyl)-alkyl, —(CH₂)₀₋₃heteroaryl, and —(C(H)(heteroaryl)-alkyl;
or R₄ and R₅ together with the nitrogen atom to which they are attached can form a carbon-containing 5-, or 6-membered saturated heterocyclic ring, optionally containing an additional heteroatom selected from N, and O, which may be optionally mono- or di-substituted with substituents selected from oxo, alkyl, alkoxy, OH, halo and CF_{3.}
R₂ can be selected from —(CH₂)₀₋₃—C(═O)—N(R₄)(R₅), —C(═O)—O—R₆, —(CH₂)₀₋₃—NR₁₄—C(═O) R₁₅, —(CH₂)₀₋₃—NR₃₄—S(═O)₂R₃₅, alkyl, cycloalkyl, heterocycloalkyl, alkoxy, —OH, halo, aryl, heteroaryl, and —CH₂—O-R₁₆, wherein:
R₄ is H or small alkyl, and
R₅ is selected from alkyl, OH, —S(═O)₂-(small alkyl), —(CH₂)₁₋₃—(C═O)—OH, —(CH₂)₀₋₃-cycloalkyl^{b}, —(CH₂)₀₋₃-heterocycloalkyl, —(CH(alkyl))-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, —(C(H)(heteroaryl))-alkyl, —(C(H)(alkyl))—(C═O)—OH, and —(C(H)(heteroaryl))—(C═O)—OR_{28;}
or R₄ and R₅ together with the nitrogen atom to which they are attached can form a carbon-containing 4-, 5-, or 6-membered saturated heterocyclic ring, optionally containing an additional heteroatom selected from N, and O, which may be optionally mono- or di-substituted with substituents selected from oxo, alkyl, alkoxy, OH, halo and CF_{3;}
R₆ is H, small alkyl, and —(CH₂)-phenyl; R₁₄ is selected from H and small alkyl;
R₁₅ is selected from H and small alkyl;
R₁₆ is selected from H, small alkyl, —(CH₂)—(C═O)—OH, heterocycloalkyl, and —(CH₂)-heteroaryl; R₃₄ is selected from H and small alkyl; and R₃₅ is selected from H, small alkyl, and cyclopropyl.
R₂ can be selected from H, —(CH₂)₀₋₃—C(═O)—N(R₄)(R₅), —C(═O)—O—R₆, —(CH₂)₀₋₃—NR₁₄—C(═O) R₁₅, —(CH₂)₀₋₃—NR₃₄—S(═O)₂R₃₅, alkyl, cycloalkyl, heterocycloalkyl, alkoxy, —OH, halo, aryl, heteroaryl, and —CH₂—O-R₁₆, wherein:
R₄ is H or small alkyl, and
R₅ is selected from alkyl, OH, —S(═O)₂-(small alkyl), —(CH₂)₁₋₃—(C═O)—OH, —(CH₂)₀₋₃-cycloalkyl^{a}, —(CH₂)₀₋₃-heterocycloalkyl, —(CH(alkyl))-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, —(C(H)(heteroaryl))-alkyl, —(C(H)(alkyl))—(C═O)—OH, and —(C(H)(heteroaryl))—(C═O)—OR_{28;}
or R₄ and R₅ together with the nitrogen atom to which they are attached can form a carbon-containing 4-, from N, and O, which may be optionally mono- or di-substituted with substituents selected from oxo, alkyl, alkoxy, OH, halo and CF₃; and R₆ is H, small alkyl, and —(CH₂)-phenyl.
R₂ can be selected from H, —(CH₂)₀₋₃—C(═O)—N(R₄)(R₅), —C(═O)—O—R₆, —(CH₂)₀₋₃—NR₁₄—C(═O) R₁₅, —(CH₂)₀₋₃—NR₃₄—S(═O)₂R₃₅, alkyl, cycloalkyl, heterocycloalkyl, alkoxy, —OH, halo, aryl, heteroaryl, and —CH₂—O-R₁₆, wherein:
R₄ is H or small alkyl, and

R₅ is selected from alkyl, —(CH₂)₀₋₃-cycloalkyl, —(CH₂)₀₋₃-heterocycloalkyl, —(CH(alkyl)-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, and —(C(H)(heteroaryl))-alkyl;
or R₄ and R₅ together with the nitrogen atom to which they are attached can form a carbon-containing 5-, or 6-membered saturated heterocyclic ring, optionally containing an additional heteroatom selected from N, and O, which may be optionally mono- or di-substituted with substituents selected from oxo, alkyl, alkoxy, OH, halo and CF₃; and R₆ is H, small alkyl, and —(CH₂)-phenyl.
R₂ can be selected from H, —(CH₂)₀₋₃—C(═O)—N(R₄)(R₅), —C(═O)—O—R₆, —(CH₂)₀₋₃—NR₁₄—C(═O) R₁₅, alkyl, cycloalkyl, heterocycloalkyl, alkoxy, —OH, halo, aryl, heteroaryl, and —CH₂—O—R₁₆, wherein:
R₄ is H or small alkyl, and
R₅ is selected from alkyl, —(CH₂)₀₋₃-cycloalkyl, —(CH₂)₀₋₃-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, and —(C(H)(heteroaryl))-alkyl;
or R₄ and R₅ together with the nitrogen atom to which they are attached can form a carbon-containing 5-, or 6-membered saturated heterocyclic ring, optionally containing an additional heteroatom selected from N, and O, which may be optionally mono- or di-substituted with substituents selected from oxo, alkyl, alkoxy, OH, halo and CF₃; and R₆ is H, small alkyl, and —(CH₂)-phenyl.
R₂ can be selected from —(CH₂)₀₋₃—C(═O)—N(R₄)(R₅), —(CH₂)₀₋₃—NR₁₄—C(═O) R₁₅, —(CH₂)₀₋₃—NR₃₄—S(═O)₂R₃₅, alkyl, cycloalkyl, heterocycloalkyl, alkoxy, —OH, halo, aryl, heteroaryl, and —CH₂—O—R₁₆, wherein:
R₄ is H or small alkyl, and
R₅ is selected from alkyl, OH, —S(═O)₂-(small alkyl), —(CH₂)₁₋₃—(C═O)—OH, —(CH₂)₀₋₃-cycloalkyl^{b}, —(CH₂)₀₋₃-heterocycloalkyl, —(CH(alkyl))-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, —(C(H)(heteroaryl))-alkyl, —(C(H)(alkyl))—(C═O)—OH, and —(C(H)(heteroaryl))—(C═O)—OR_{28;}
or R₄ and R₅ together with the nitrogen atom to which they are attached can form a carbon-containing 4-, 5-, or 6-membered saturated heterocyclic ring, optionally containing an additional heteroatom selected from N, and O, which may be optionally mono- or di-substituted with substituents selected from oxo, alkyl, alkoxy, OH, halo and CF₃; R₆ is H, small alkyl, and —(CH₂)-phenyl; R₁₄ is selected from H and small alkyl; R₁₅ is selected from H and small alkyl;
R₁₆ is selected from H, small alkyl, —(CH₂)—(C═O)—OH, heterocycloalkyl, and —(CH₂)-heteroaryl; R₃₄ is selected from H and small alkyl; and R₃₅ is selected from H, small alkyl, and cyclopropyl.
R₂ can be selected from H, —(CH₂)₀₋₃—C(═O)—N(R₄)(R₅), —(CH₂)₀₋₃—NR₁₄—C(═O) R₁₅, —(CH₂)₀₋₃—NR₃₄—S(═O)₂R₃₅, alkyl, cycloalkyl, heterocycloalkyl, alkoxy, —OH, halo, aryl, heteroaryl, and —CH₂—O—R₁₆, wherein: R₄ is H or small alkyl, and

R₅ is selected from alkyl, OH, —S(═O)₂-(small alkyl), —(CH₂)₁₋₃—(C═O)—OH, —(CH₂)₀₋₃-cycloalkyl^{a}, —(CH₂)₀₋₃-heterocycloalkyl, —(CH(alkyl))-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, —(C(H)(heteroaryl))-alkyl, —(C(H)(alkyl))—(C═O)—OH, and —(C(H)(heteroaryl))—(C═O)—OR_{28;}
or R₄ and R₅ together with the nitrogen atom to which they are attached can form a carbon-containing 4-, 5-, or 6-membered saturated heterocyclic ring, optionally containing an additional heteroatom selected from N, and O, which may be optionally mono- or di-substituted with substituents selected from oxo, alkyl, alkoxy, OH, halo and CF_{3.}
R₂ can be selected from H, —(CH₂)₀₋₃—C(═O)—N(R₄)(R₅), —(CH₂)₀₋₃—NR₁₄—C(═O) R₁₅, —(CH₂)₀₋₃—NR₃₄—S(═O)₂R₃₅, alkyl, cycloalkyl, heterocycloalkyl, alkoxy, —OH, halo, aryl, heteroaryl, and —CH₂—O—R₁₆, wherein:
R₄ is H or small alkyl, and
R₅ is selected from alkyl, —(CH₂)₀₋₃-cycloalkyl, —(CH₂)₀₋₃-heterocycloalkyl, —(CH(alkyl))-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, and —(C(H)(heteroaryl))-alkyl;
or R₄ and R₅ together with the nitrogen atom to which they are attached can form a carbon-containing 5-, or 6-membered saturated heterocyclic ring, optionally containing an additional heteroatom selected from N, and O, which may be optionally mono- or di-substituted with substituents selected from oxo, alkyl, alkoxy, OH, halo and CF_{3.}
R₂ can be selected from H, —(CH₂)₀₋₃—C(═O)—N(R₄)(R₅), —(CH₂)₀₋₃—NR₁₄—C(═O) R₁₅, alkyl, cycloalkyl, heterocycloalkyl, alkoxy, —OH, halo, aryl, heteroaryl, and —CH₂—O—R₁₆, wherein:
R₄ is H or small alkyl, and
R₅ is selected from alkyl, —(CH₂)₀₋₃-cycloalkyl, —(CH₂)₀₋₃-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, and —(C(H)(heteroaryl))-alkyl;
or R₄ and R₅ together with the nitrogen atom to which they are attached can form a carbon-containing 5-, or 6-membered saturated heterocyclic ring, optionally containing an additional heteroatom selected from N, and O, which may be optionally mono- or di-substituted with substituents selected from oxo, alkyl, alkoxy, OH, halo and CF_{3.}
R₂ can be selected from —(CH₂)₀₋₃—C(═O)—N(R₄)(R₅), —C(═O)—O—R₆,—CH₂—NH—C(═O) CH₃,

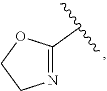 [SMILES: *C1=NCCO1]

—CH₂—NH—S(═O)₂R₃₅,—CH₂—O—R₁₆,—CH₂—OH, —CN, and wherein:
R₄ is H or small alkyl, and

R₅ is selected from alkyl, OH, —S(═O)₂-(small alkyl), —(CH₂)₁₋₃—(C═O)—OH, —(CH₂)₀₋₃-cycloalkyl^{b}, —(CH₂)₀₋₃-heterocycloalkyl, —(CH(alkyl))-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, —(C(H)(heteroaryl))-alkyl, —(C(H)(alkyl))—(C═O)—OH, and —(C(H)(heteroaryl))—(C═O)—OR_{28;}
or R₄ and R₅ together with the nitrogen atom to which they are attached can form a carbon-containing 4-, 5-, or 6-membered saturated heterocyclic ring, optionally containing an additional heteroatom selected from N, and O, which may be optionally mono- or di-substituted with substituents selected from oxo, alkyl, alkoxy, OH, halo and CF₃; R₆ is H, small alkyl, and —(CH₂)-phenyl;
R₁₆ is selected from H, small alkyl, —(CH₂)—(C═O)—OH, heterocycloalkyl, and —(CH₂)-heteroaryl; and R₃₅ is selected from H, small alkyl, and cyclopropyl.
R₂ can be selected from —(CH₂)₀₋₃—C(═O)—N(R₄)(R₅), —C(═O)—O—R₆,—CH₂—NH—C(═O) CH₃,

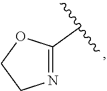 [SMILES: *C1=NCCO1]

—CH₂—NH—S(═O)₂(cyclopropyl),—CH₂—O—R₁₆,—CH₂—OH, —CN, and wherein:
R₄ is H or small alkyl, and

R₅ is selected from alkyl, OH, —S(═O)₂-(small alkyl), —(CH₂)₁₋₃—(C═O)—OH, —(CH₂)₀₋₃-cycloalkyl^{a}, —(CH₂)₀₋₃-heterocycloalkyl, —(CH(alkyl))-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, —(C(H)(heteroaryl))-alkyl, —(C(H)(alkyl))—(C═O)—OH, and —(C(H)(heteroaryl))—(C═O)—OR_{28;}
or R₄ and R₅ together with the nitrogen atom to which they are attached can form a carbon-containing 4-, 5-, or 6-membered saturated heterocyclic ring, optionally containing an additional heteroatom selected from N, and O, which may be optionally mono- or di-substituted with substituents selected from oxo, alkyl, alkoxy, OH, halo and CF₃; and
R₆ is H, small alkyl, and —(CH₂)-phenyl.
R₂ can be selected from —(CH₂)₀₋₃—C(═O)—N(R₄)(R₅), —C(═O)—O—R₆,—CH₂—NH—S(═O)₂(cyclopropyl),—CH₂—O—CH₃, —CH₂—OH, and —CN, wherein:
R₄ is H or small alkyl, and

R₅ is selected from alkyl, —(CH₂)₀₋₃-cycloalkyl, —(CH₂)₀₋₃-heterocycloalkyl, —(CH(alkyl)-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, and —(C(H)(heteroaryl))-alkyl;
or R₄ and R₅ together with the nitrogen atom to which they are attached can form a carbon-containing 5-, or 6-membered saturated heterocyclic ring, optionally containing an additional heteroatom selected from N, and O, which may be optionally mono- or di-substituted with substituents selected from oxo, alkyl, alkoxy, OH, halo and CF₃; and R₆ is H, small alkyl, and —(CH₂)-phenyl.
R₂ can be selected from —(CH₂)₀₋₃—C(═O)—N(R₄)(R₅), —C(═O)—O—R₆,—CH₂—O—CH₃, and —CN, wherein:
R₄ is H or small alkyl, and
R₅ is selected from alkyl, —(CH₂)₀₋₃-cycloalkyl, —(CH₂)₀₋₃-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, and —(C(H)(heteroaryl))-alkyl;
or R₄ and R₅ together with the nitrogen atom to which they are attached can form a carbon-containing 5-, or 6-membered saturated heterocyclic ring, optionally containing an additional heteroatom selected from N, and O, which may be optionally mono- or di-substituted with substituents selected from oxo, alkyl, alkoxy, OH, halo and CF₃; and
R₆ is H, small alkyl, and —(CH₂)-phenyl.
R₂ can be selected from —(CH₂)₀₋₃—C(═O)—N(R₄)(R₅), —C(═O)—O—R₆,—CH₂—NH—C(═O) CH₃,

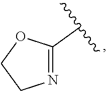 [SMILES: *C1=NCCO1]

—CH₂—NH—S(═O)₂R₃₅,—CH₂—O—R₁₆,—CH₂—OH, and wherein:
R₄ is H or small alkyl, and

R₅ is selected from alkyl, OH, —S(═O)₂-(small alkyl), —(CH₂)₁₋₃—(C═O)—OH, —(CH₂)₀₋₃-cycloalkyl^{b}, —(CH₂)₀₋₃-heterocycloalkyl, —(CH(alkyl))-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, —(C(H)(heteroaryl))-alkyl, —(C(H)(alkyl))—(C═O)—OH, and —(C(H)(heteroaryl))—(C═O)—OR_{28;}
or R₄ and R₅ together with the nitrogen atom to which they are attached can form a carbon-containing 4-, 5-, or 6-membered saturated heterocyclic ring, optionally containing an additional heteroatom selected from N, and O, which may be optionally mono- or di-substituted with substituents selected from oxo, alkyl, alkoxy, OH, halo and CF_{3;}
R₆ is H, small alkyl, and —(CH₂)-phenyl;
R₁₆ is selected from H, small alkyl, —(CH₂)—(C═O)—OH, heterocycloalkyl, and —(CH₂)-heteroaryl; and R₃₅ is selected from H, small alkyl, and cyclopropyl.
R₂ can be selected from —(CH₂)₀₋₃—C(═O)—N(R₄)(R₅), —C(═O)—O—R₆,—CH₂—NH—C(═O) CH₃,

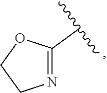 [SMILES: *C1=NCCO1]

—CH₂—NH—S(═O)₂(cyclopropyl),—CH₂—O—R₁₆,—CH₂—OH, and wherein:
R₄ is H or small alkyl, and

R₅ is selected from alkyl, OH, —S(═O)₂-(small alkyl), —(CH₂)₁₋₃—(C═O)—OH, —(CH₂)₀₋₃-cycloalkyl^{a}, —(CH₂)₀₋₃-heterocycloalkyl, —(CH(alkyl))-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, —(C(H)(heteroaryl))-alkyl, —(C(H)(alkyl))—(C═O)—OH, and —(C(H)(heteroaryl))—(C═O)—OR_{28;}
or R₄ and R₅ together with the nitrogen atom to which they are attached can form a carbon-containing 4-, 5-, or 6-membered saturated heterocyclic ring, optionally containing an additional heteroatom selected from N, and O, which may be optionally mono- or di-substituted with substituents selected from oxo, alkyl, alkoxy, OH, halo and CF₃; and
R₆ is H, small alkyl, and —(CH₂)-phenyl.
R₂ can be selected from —(CH₂)₀₋₃—C(═O)—N(R₄)(R₅), —C(═O)—O—R₆,—CH₂—NH—S(═O)₂(cyclopropyl),—CH₂—O—CH₃, and —CH₂—OH, wherein:
R₄ is H or small alkyl, and
R₅ is selected from alkyl, —(CH₂)₀₋₃-cycloalkyl, —(CH₂)₀₋₃-heterocycloalkyl, —(CH(alkyl))-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, and —(C(H)(heteroaryl))-alkyl;
or R₄ and R₅ together with the nitrogen atom to which they are attached can form a carbon-containing 5-, or 6-membered saturated heterocyclic ring, optionally containing an additional heteroatom selected from N, and O, which may be optionally mono- or di-substituted with substituents selected from oxo, alkyl, alkoxy, OH, halo and CF₃; and
Ro is H, small alkyl, and —(CH₂)-phenyl.
R₂ can be selected from —(CH₂)₀₋₃—C(═O)—N(R₄)(R₅), —C(═O)—O—R₆, and —CH₂—O—CH₃, wherein:
R₄ is H or small alkyl, and
R₅ is selected from alkyl, —(CH₂)₀₋₃-cycloalkyl, —(CH₂)₀₋₃-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, and —(C(H)(heteroaryl))-alkyl;
or R₄ and R₅ together with the nitrogen atom to which they are attached can form a carbon-containing 5-, or 6-membered saturated heterocyclic ring, optionally containing an additional heteroatom selected from N, and O, which may be optionally mono- or di-substituted with substituents selected from oxo, alkyl, alkoxy, OH, halo and CF₃; and R₆ is H, small alkyl, and —(CH₂)-phenyl.
R₂ can be selected from —(CH₂)₀₋₃—C(═O)—N(R₄)(R₅),—CH₂—NH—C(═O) CH₃,—CH₂—NH—S(═O)₂R₃₅,

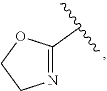 [SMILES: *C1=NCCO1]

—CH₂—O—R₁₆,—CH₂—OH, —CN, and wherein:
R₄ is H or small alkyl, and
R₅ is selected from alkyl, OH, —S(═O)₂-(small alkyl), —(CH₂)₁₋₃—(C═O)—OH, —(CH₂)₀₋₃-cycloalkyl^{b}, —(CH₂)₀₋₃-heterocycloalkyl, —(CH(alkyl))-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, —(C(H)(heteroaryl))-alkyl, —(C(H)(alkyl))—(C═O)—OH, and —(C(H)(heteroaryl))—(C═O)—OR_{28;}
or R₄ and R₅ together with the nitrogen atom to which they are attached can form a carbon-containing 4-, 5-, or 6-membered saturated heterocyclic ring, optionally containing an additional heteroatom selected from N, and O, which may be optionally mono- or di-substituted with substituents selected from oxo, alkyl, alkoxy, OH, halo and CF₃; R₆ is H, small alkyl, and —(CH₂)-phenyl;
R₁₆ is selected from H, small alkyl, —(CH₂)—(C═O)—OH, heterocycloalkyl, and —(CH₂)-heteroaryl; and R₃₅ is selected from H, small alkyl, and cyclopropyl.
R₂ can be selected from —(CH₂)₀₋₃—C(═O)—N(R₄)(R₅),—CH₂—NH—C(═O) CH₃,—CH₂—NH—S(═O)₂(cyclopropyl),

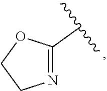 [SMILES: *C1=NCCO1]

—CH₂—O—R₁₆,—CH₂—OH, —CN, and wherein:
R₄ is H or small alkyl, and

R₅ is selected from alkyl, OH, —S(═O)₂-(small alkyl), —(CH₂)₁₋₃—(C═O)—OH, —(CH₂)₀₋₃-cycloalkyl^{a}, —(CH₂)₀₋₃-heterocycloalkyl, —(CH(alkyl))-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, —(C(H)(heteroaryl))-alkyl, —(C(H)(alkyl))—(C═O)—OH, and —(C(H)(heteroaryl))—(C═O)—OR_{28;}
or R₄ and R₅ together with the nitrogen atom to which they are attached can form a carbon-containing 4-, 5-, or 6-membered saturated heterocyclic ring, optionally containing an additional heteroatom selected from N, and O, which may be optionally mono- or di-substituted with substituents selected from oxo, alkyl, alkoxy, OH, halo and CF_{3.}
R₂ can be selected from —(CH₂)₀₋₃—C(═O)—N(R₄)(R₅),—CH₂—NH—S(═O)₂(cyclopropyl),—CH₂—O—CH₃,—CH₂—OH, and —CN, wherein:
R₄ is H or small alkyl, and

R₅ is selected from alkyl, —(CH₂)₀₋₃-cycloalkyl, —(CH₂)₀₋₃-heterocycloalkyl, —(CH(alkyl)-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, and —(C(H)(heteroaryl))-alkyl;
or R₄ and R₅ together with the nitrogen atom to which they are attached can form a carbon-containing 5-, or 6-membered saturated heterocyclic ring, optionally containing an additional heteroatom selected from N, and O, which may be optionally mono- or di-substituted with substituents selected from oxo, alkyl, alkoxy, OH, halo and CF_{3.}
R₂ can be selected from —(CH₂)₀₋₃—C(═O)—N(R₄)(R₅),—CH₂—O—CH₃,—CH₂—OH, and —CN, wherein:
R₄ is H or small alkyl, and
R₅ is selected from alkyl, —(CH₂)₀₋₃-cycloalkyl, —(CH₂)₀₋₃-heterocycloalkyl, —(CH₂)₀₋₃-polycycloalkyl, —(CH₂)₀₋₃-aryl, —(C(H)(phenyl)-alkyl, —(CH₂)₀₋₃heteroaryl, and —(C(H)(heteroaryl)-alkyl;
or R₄ and R₅ together with the nitrogen atom to which they are attached can form a carbon-containing 5-, or 6-membered saturated heterocyclic ring, optionally containing an additional heteroatom selected from N, and O, which may be optionally mono- or di-substituted with substituents selected from oxo, alkyl, alkoxy, OH, halo and CF_{3.}
R₂ can be selected from —(CH₂)₀₋₃—C(═O)—N(R₄)(R₅),—CH₂—NH—C(═O) CH₃,—CH₂—NH—S(═O)₂R₃₅,

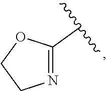 [SMILES: *C1=NCCO1]

—CH₂—O—R₁₆,—CH₂—OH, and wherein:
R₄ is H or small alkyl, and
R₅ is selected from alkyl, OH, —S(═O)₂-(small alkyl), —(CH₂)₁₋₃—(C═O)—OH, —(CH₂)₀₋₃-cycloalkyl^{b}, —(CH₂)₀₋₃-heterocycloalkyl, —(CH(alkyl))-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, —(C(H)(heteroaryl))-alkyl, —(C(H)(alkyl))—(C═O)—OH, and —(C(H)(heteroaryl))—(C═O)—OR_{28;}
or R₄ and R₅ together with the nitrogen atom to which they are attached can form a carbon-containing 4-, 5-, or 6-membered saturated heterocyclic ring, optionally containing an additional heteroatom selected from N, and O, which may be optionally mono- or di-substituted with substituents selected from oxo, alkyl, alkoxy, OH, halo and CF₃; R₆ is H, small alkyl, and —(CH₂)-phenyl; R₁₆ is selected from H, small alkyl, —(CH₂)—(C═O)—OH, heterocycloalkyl, and —(CH₂)-heteroaryl; and R₃₅ is selected from H, small alkyl, and cyclopropyl.
R₂ can be selected from —(CH₂)₀₋₃—C(═O)—N(R₄)(R₅),—CH₂—NH—C(═O) CH₃,—CH₂—NH—S(═O)₂(cyclopropyl),

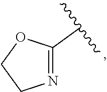 [SMILES: *C1=NCCO1]

—CH₂—O—R₁₆,—CH₂—OH, and wherein:
R₄ is H or small alkyl, and

R₅ is selected from alkyl, OH, —S(═O)₂-(small alkyl), —(CH₂)₁₋₃—(C═O)—OH, —(CH₂)₀₋₃-cycloalkyl^{a}, —(CH₂)₀₋₃-heterocycloalkyl, —(CH(alkyl))-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, —(C(H)(heteroaryl))-alkyl, —(C(H)(alkyl))—(C═O)—OH, and —(C(H)(heteroaryl))—(C═O)—OR_{28;}
or R₄ and R₅ together with the nitrogen atom to which they are attached can form a carbon-containing 4-, 5-, or 6-membered saturated heterocyclic ring, optionally containing an additional heteroatom selected from N, and O, which may be optionally mono- or di-substituted with substituents selected from oxo, alkyl, alkoxy, OH, halo and CF_{3.}
R₂ can be selected from —(CH₂)₀₋₃—C(═O)—N(R₄)(R₅),—CH₂—NH—S(═O)₂(cyclopropyl),—CH₂—O—CH₃, and —CH₂—OH, wherein:
R₄ is H or small alkyl, and

R₅ is selected from alkyl, —(CH₂)₀₋₃-cycloalkyl, —(CH₂)₀₋₃-heterocycloalkyl, —(CH(alkyl)-heterocycloalkyl, —(CH₂)₀₋₃-polycycloalkyl, —(CH₂)₀₋₃heteroaryl, —(CH₂)₀₋₃-aryl, —(C(H)(phenyl))-alkyl, and —(C(H)(heteroaryl))-alkyl;
or R₄ and R₅ together with the nitrogen atom to which they are attached can form a carbon-containing 5-, or 6-membered saturated heterocyclic ring, optionally containing an additional heteroatom selected from N, and O, which may be optionally mono- or di-substituted with substituents selected from oxo, alkyl, alkoxy, OH, halo and CF_{3.}
R₂ can be selected from —(CH₂)₀₋₃—C(═O)—N(R₄)(R₅), and —CH₂—O—CH₃, wherein:
R₄ is H or small alkyl, and
R₅ is selected from alkyl, —(CH₂)₀₋₃-cycloalkyl, —(CH₂)₀₋₃-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, and —(C(H)(heteroaryl))-alkyl;
or R₄ and R₅ together with the nitrogen atom to which they are attached can form a carbon-containing 5-, or 6-membered saturated heterocyclic ring, optionally containing an additional heteroatom selected from N, and O, which may be optionally mono- or di-substituted with substituents selected from oxo, alkyl, alkoxy, OH, halo and CF_{3.}

Preferably, R₂ is selected from —C(═O)—N(R₄)(R₅), —C(═O)—O—R₆,—CH₂—NH—C(═O) CH₃,

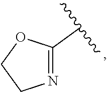 [SMILES: *C1=NCCO1]

—CH₂—NH—S(═O)₂R₃₅,—CH₂—O—R₁₆,—CH₂—OH, —CN, and wherein:
R₄ is H or small alkyl, and
R₅ is selected from alkyl, OH, —S(═O)₂-(small alkyl), —(CH₂)₁₋₃—(C═O)—OH, —(CH₂)₀₋₃-cycloalkyl^{b}, —(CH₂)₀₋₃-heterocycloalkyl, —(CH(alkyl)-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, —(C(H)(heteroaryl))-alkyl, —(C(H)(alkyl))—(C═O)—OH, and —(C(H)(heteroaryl))—(C═O)—OR_{28;}
or R₄ and R₅ together with the nitrogen atom to which they are attached can form a carbon-containing 4-, 5-, or 6-membered saturated heterocyclic ring, optionally containing an additional heteroatom selected from N, and O, which may be optionally mono- or di-substituted with substituents selected from oxo, alkyl, alkoxy, OH, halo and CF₃; and
R₆ is H, small alkyl, and —(CH₂)-phenyl;
R₁₆ is selected from H, small alkyl, —(CH₂)—(C═O)—OH, heterocycloalkyl, and —(CH₂)-heteroaryl; and R₃₅ is selected from H, small alkyl, and cyclopropyl.
R₂ can be selected from —C(═O)—N(R₄)(R₅), —C(═O)—O—R₆,—CH₂—NH—C(═O) CH₃,

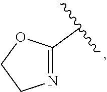 [SMILES: *C1=NCCO1]

—CH₂—NH—S(═O)₂(cyclopropyl),—CH₂—O—R₁₆,—CH₂—OH, —CN, and wherein:
R₄ is H or small alkyl, and
R₅ is selected from alkyl, OH, —S(═O)₂-(small alkyl), —(CH₂)₁₋₃—(C═O)—OH, —(CH₂)₀₋₃-cycloalkyl^{a}, —(CH₂)₀₋₃-heterocycloalkyl, —(CH(alkyl)-heterocycloalkyl, —(CH₂)₀₋₃-polycycloalkyl, —(CH₂)₀₋₃-aryl, —(C(H)(phenyl)-alkyl, —(CH₂)₀₋₃heteroaryl, —(C(H)(heteroaryl)-alkyl, —(C(H)(alkyl)—(C═O)—OH, and —(C(H)(heteroaryl))—(C═O)—OR_{28;}
or R₄ and R₅ together with the nitrogen atom to which they are attached can form a carbon-containing 4-, 5-, or 6-membered saturated heterocyclic ring, optionally containing an additional heteroatom selected from N, and O, which may be optionally mono- or di-substituted with substituents selected from oxo, alkyl, alkoxy, OH, halo and CF₃; and R₆ is H, small alkyl, and —(CH₂)-phenyl.
R₂ can be selected from —C(═O)—N(R₄)(R₅), —C(═O)—O—R₆,—CH₂—NH—S(═O)₂(cyclopropyl), —CH₂—O—CH₃,—CH₂—OH, and —CN, wherein:
R₄ is H or small alkyl, and
R₅ is selected from alkyl, —(CH₂)₀₋₃-cycloalkyl, —(CH₂)₀₋₃-heterocycloalkyl, —(CH(alkyl))-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, and —(C(H)(heteroaryl))-alkyl;
or R₄ and R₅ together with the nitrogen atom to which they are attached can form a carbon-containing 5-, or 6-membered saturated heterocyclic ring, optionally containing an additional heteroatom selected from N, and O, which may be optionally mono- or di-substituted with substituents selected from oxo, alkyl, alkoxy, OH, halo and CF₃; and Re is H, small alkyl, and —(CH₂)-phenyl.
R₂ can be selected from —C(═O)—N(R₄)(R₅), —C(═O)—O—R₆,—CH₂—O—CH₃, and —CN, wherein:
R₄ is H or small alkyl, and
R₅ is selected from alkyl, —(CH₂)₀₋₃-cycloalkyl, —(CH₂)₀₋₃-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, and —(C(H)(heteroaryl))-alkyl;
or R₄ and R₅ together with the nitrogen atom to which they are attached can form a carbon-containing 5-, or 6-membered saturated heterocyclic ring, optionally containing an additional heteroatom selected from N, and O, which may be optionally mono- or di-substituted with substituents selected from oxo, alkyl, alkoxy, OH, halo and CF₃; and R₆ is H, small alkyl, and —(CH₂)-phenyl.
R₂ can be selected from —C(═O)—N(R₄)(R₅), —C(═O)—O—R₆,—CH₂—NH—C(═O) CH₃,—CH₂—NH—S(═O)₂R₃₅,

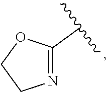 [SMILES: *C1=NCCO1]

—CH₂—O—R₁₆,—CH₂—OH, and wherein:
R₄ is H or small alkyl, and
R₅ is selected from alkyl, OH, —S(═O)₂-(small alkyl), —(CH₂)₁₋₃—(C═O)—OH, —(CH₂)₀₋₃-cycloalkyl^{b}, —(CH₂)₀₋₃-heterocycloalkyl, —(CH(alkyl))-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, —(C(H)(heteroaryl))-alkyl, —(C(H)(alkyl))—(C═O)—OH, and —(C(H)(heteroaryl))—(C═O)—OR_{28;}
or R₄ and R₅ together with the nitrogen atom to which they are attached can form a carbon-containing 4-, 5-, or 6-membered saturated heterocyclic ring, optionally containing an additional heteroatom selected from N, and O, which may be optionally mono- or di-substituted with substituents selected from oxo, alkyl, alkoxy, OH, halo and CF_{3;}
R₆ is H, small alkyl, and —(CH₂)-phenyl; R₁₆ is selected from H, small alkyl, —(CH₂)—(C═O)—OH, heterocycloalkyl, and —(CH₂)-heteroaryl; and R₃₅ is selected from H, small alkyl, and cyclopropyl.
R₂ can be selected from —C(═O)—N(R₄)(R₅), —C(═O)—O—R₆,—CH₂—NH—C(═O) CH₃,—CH₂—NH—S(═O)₂(cyclopropyl),

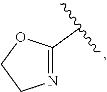 [SMILES: *C1=NCCO1]

—CH₂—O—R₁₆,—CH₂—OH, and wherein:
R₄ is H or small alkyl, and

R₅ is selected from alkyl, OH, —S(═O)₂-(small alkyl), —(CH₂)₁₋₃—(C═O)—OH, —(CH₂)₀₋₃-cycloalkyl^{a}, —(CH₂)₀₋₃-heterocycloalkyl, —(CH(alkyl))-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, —(C(H)(heteroaryl))-alkyl, —(C(H)(alkyl))—(C═O)—OH, and —(C(H)(heteroaryl))—(C═O)—OR_{28;}
or R₄ and R₅ together with the nitrogen atom to which they are attached can form a carbon-containing 4-, from N, and O, which may be optionally mono- or di-substituted with substituents selected from oxo, alkyl, alkoxy, OH, halo and CF₃; and
R₆ is H, small alkyl, and —(CH₂)-phenyl.
R₂ can be selected from —C(═O)—N(R₄)(R₅), —C(═O)—O—R₆,—CH₂—NH—S(═O)₂(cyclopropyl),—CH₂—O—CH₃, and —CH₂—OH, wherein:
R₄ is H or small alkyl, and

R₅ is selected from alkyl, —(CH₂)₀₋₃-cycloalkyl, —(CH₂)₀₋₃-heterocycloalkyl, —(CH(alkyl)-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, and —(C(H)(heteroaryl))-alkyl;
or R₄ and R₅ together with the nitrogen atom to which they are attached can form a carbon-containing 5-, or 6-membered saturated heterocyclic ring, optionally containing an additional heteroatom selected from N, and O, which may be optionally mono- or di-substituted with substituents selected from oxo, alkyl, alkoxy, OH, halo and CF₃; and
R₆ is H, small alkyl, and —(CH₂)-phenyl.
R₂ can be selected from —C(═O)—N(R₄)(R₅), —C(═O)—O—R₆, and —CH₂—O—CH₃, wherein: R₄ is H or small alkyl, and
R₅ is selected from —(CH₂)₀₋₃-heterocycloalkyl, —(CH₂)₀₋₃-polycycloalkyl, —(CH₂)₀₋₃-aryl, alkyl, —(CH₂)₀₋₃-cycloalkyl, —(C(H)(phenyl))-alkyl, —(CH₂)₀₋₃heteroaryl, and —(C(H)(heteroaryl))-alkyl;
or R₄ and R₅ together with the nitrogen atom to which they are attached can form a carbon-containing 5-, or 6-membered saturated heterocyclic ring, optionally containing an additional heteroatom selected from N, and O, which may be optionally mono- or di-substituted with substituents selected from oxo, alkyl, alkoxy, OH, halo and CF₃; and R₆ is H, small alkyl, and —(CH₂)-phenyl.
R₂ can be selected from —C(═O)—N(R₄)(R₅),—CH₂—NH—C(═O) CH₃,—CH₂—NH—S(═O)₂R₃₅,—CH₂—O—R₁₆,

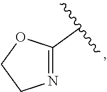 [SMILES: *C1=NCCO1]

—CH₂—OH, —CN, and wherein:
R₄ is H or small alkyl, and

R₅ is selected from alkyl, OH, —S(═O)₂-(small alkyl), —(CH₂)₁₋₃—(C═O)—OH, —(CH₂)₀₋₃-cycloalkyl^{b}, —(CH₂)₀₋₃-heterocycloalkyl, —(CH(alkyl))-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, —(C(H)(heteroaryl))-alkyl, —(C(H)(alkyl))—(C═O)—OH, and —(C(H)(heteroaryl))—(C═O)—OR_{28;}
or R₄ and R₅ together with the nitrogen atom to which they are attached can form a carbon-containing 4-, from N, and O, which may be optionally mono- or di-substituted with substituents selected from oxo, alkyl, alkoxy, OH, halo and CF_{3;}
R₆ is H, small alkyl, and —(CH₂)-phenyl;
R₁₆ is selected from H, small alkyl, —(CH₂)—(C═O)—OH, heterocycloalkyl, and —(CH₂)-heteroaryl; and
R₃₅ is selected from H, small alkyl, and cyclopropyl.
R₂ can be selected from —C(═O)—N(R₄)(R₅),—CH₂—NH—C(═O) CH₃,—CH₂—NH—S(═O)₂(cyclopropyl),—CH₂—O—R₁₆,

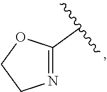 [SMILES: *C1=NCCO1]

—CH₂—OH, —CN, and wherein:
R₄ is H or small alkyl, and

R₅ is selected from alkyl, OH, —S(═O)₂-(small alkyl), —(CH₂)₁₋₃—(C═O)—OH, —(CH₂)₀₋₃-cycloalkyl^{a}, —(CH₂)₀₋₃-heterocycloalkyl, —(CH(alkyl))-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, —(C(H)(heteroaryl))-alkyl, —(C(H)(alkyl))—(C═O)—OH, and —(C(H)(heteroaryl))—(C═O)—OR_{28;}
or R₄ and R₅ together with the nitrogen atom to which they are attached can form a carbon-containing 4-, 5-, or 6-membered saturated heterocyclic ring, optionally containing an additional heteroatom selected from N, and O, which may be optionally mono- or di-substituted with substituents selected from oxo, alkyl, alkoxy, OH, halo and CF_{3.}
R₂ can be selected from —C(═O)—N(R₄)(R₅),—CH₂—NH—S(═O)₂(cyclopropyl),—CH₂—O—CH₃,—CH₂—OH, and —CN, wherein:
R₄ is H or small alkyl, and

R₅ is selected from alkyl, —(CH₂)₀₋₃-cycloalkyl, —(CH₂)₀₋₃-heterocycloalkyl, —(CH(alkyl)-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, and —(C(H)(heteroaryl))-alkyl;
or R₄ and R₅ together with the nitrogen atom to which they are attached can form a carbon-containing 5-, or 6-membered saturated heterocyclic ring, optionally containing an additional heteroatom selected from N, and O, which may be optionally mono- or di-substituted with substituents selected from oxo, alkyl, alkoxy, OH, halo and CF_{3.}
R₂ can be selected from —C(═O)—N(R₄)(R₅),—CH₂—O—CH₃, and —CN, wherein: R₄ is H or small alkyl, and
R₅ selected —(CH₂)₀₋₃-cycloalkyl, —(CH₂)₀₋₃-heterocycloalkyl, —(CH₂)₀₋₃-polycycloalkyl, alkyl, —(C(H)(phenyl))-alkyl, —(CH₂)₀₋₃heteroaryl, and —(C(H)(heteroaryl))-alkyl;
is from —(CH₂)₀₋₃-aryl, or R₄ and R₅ together with the nitrogen atom to which they are attached can form a carbon-containing 5-, or 6-membered saturated heterocyclic ring, optionally containing an additional heteroatom selected from N, and O, which may be optionally mono- or di-substituted with substituents selected from oxo, alkyl, alkoxy, OH, halo and CF_{3.}
R₂ can be selected from —C(═O)—N(R₄)(R₅),—CH₂—NH—C(═O) CH₃,—CH₂—NH—S(═O)₂R₃₅,—CH₂—O—R₁₆,

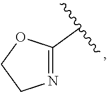 [SMILES: *C1=NCCO1]

—CH₂—OH, and wherein:
R₄ is H or small alkyl, and

R₅ is selected from alkyl, OH, —S(═O)₂-(small alkyl), —(CH₂)₁₋₃—(C═O)—OH, —(CH₂)₀₋₃-cycloalkyl^{b}, —(CH₂)₀₋₃-heterocycloalkyl, —(CH(alkyl))-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, —(C(H)(heteroaryl))-alkyl, —(C(H)(alkyl))—(C═O)—OH, and —(C(H)(heteroaryl))—(C═O)—OR_{28;}
or R₄ and R₅ together with the nitrogen atom to which they are attached can form a carbon-containing 4-, 5-, or 6-membered saturated heterocyclic ring, optionally containing an additional heteroatom selected from N, and O, which may be optionally mono- or di-substituted with substituents selected from oxo, alkyl, alkoxy, OH, halo and CF_{3;}
R₆ is H, small alkyl, and —(CH₂)-phenyl;
R₁₆ is selected from H, small alkyl, —(CH₂)—(C═O)—OH, heterocycloalkyl, and —(CH₂)-heteroaryl; and R₃₅ is selected from H, small alkyl, and cyclopropyl.
R₂ can be selected from —C(═O)—N(R₄)(R₅),—CH₂—NH—C(═O) CH₃,—CH₂—NH—S(═O)₂(cyclopropyl),—CH₂—O—R₁₆,

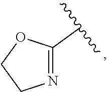 [SMILES: *C1=NCCO1]

—CH₂—OH, and wherein:
R₄ is H or small alkyl, and

R₅ is selected from alkyl, OH, —S(═O)₂-(small alkyl), —(CH₂)₁₋₃—(C═O)—OH, —(CH₂)₀₋₃-cycloalkyl^{a}, —(CH₂)₀₋₃-heterocycloalkyl, —(CH(alkyl))-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, —(C(H)(heteroaryl))-alkyl, —(C(H)(alkyl))—(C═O)—OH, and —(C(H)(heteroaryl))—(C═O)—OR_{28;}
or R₄ and R₅ together with the nitrogen atom to which they are attached can form a carbon-containing 4-, from N, and O, which may be optionally mono- or di-substituted with substituents selected from oxo, alkyl, alkoxy, OH, halo and CF_{3.}
R₂ can be selected from —C(═O)—N(R₄)(R₅),—CH₂—NH—S(═O)₂(cyclopropyl),—CH₂—O—CH₃, and —CH₂—OH wherein:
R₄ is H or small alkyl, and

R₅ is selected from alkyl, —(CH₂)₀₋₃-cycloalkyl, —(CH₂)₀₋₃-heterocycloalkyl, —(CH(alkyl)-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, and —(C(H)(heteroaryl))-alkyl;
or R₄ and R₅ together with the nitrogen atom to which they are attached can form a carbon-containing 5-, or 6-membered saturated heterocyclic ring, optionally containing an additional heteroatom selected from N, and O, which may be optionally mono- or di-substituted with substituents selected from oxo, alkyl, alkoxy, OH, halo and CF_{3.}
R₂ can be selected from —C(═O)—N(R₄)(R₅), and —CH₂—O—CH₃, wherein:
R₄ is H or small alkyl, and
R₅ is selected from alkyl, —(CH₂)₀₋₃-cycloalkyl, —(CH₂)₀₋₃-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, and —(C(H)(heteroaryl))-alkyl;
or R₄ and R₅ together with the nitrogen atom to which they are attached can form a carbon-containing 5-, or 6-membered saturated heterocyclic ring, optionally containing an additional heteroatom selected from N, and O, which may be optionally mono- or di-substituted with substituents selected from oxo, alkyl, alkoxy, OH, halo and CF₃.

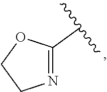 [SMILES: *C1=NCCO1]

R₂ can be selected from —C(═O)—N(R₄)(R₅),—CH₂—NH—C(═O) CH₃,—CH₂—NH—S(═O)₂R₃₅,—CH₂—OH, and wherein:
R₄ is H or small alkyl, and
R₅ is selected from alkyl, OH, —S(═O)₂-(small alkyl), —(CH₂)₁₋₃—(C═O)—OH, —(CH₂)₀₋₃-cycloalkyl^{b}, —(CH₂)₀₋₃-heterocycloalkyl, —(CH(alkyl)-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, —(C(H)(heteroaryl))-alkyl, —(C(H)(alkyl))—(C═O)—OH, and —(C(H)(heteroaryl))—(C═O)—OR_{28;}
or R₄ and R₅ together with the nitrogen atom to which they are attached can form a carbon-containing 4-, from N, and O, which may be optionally mono- or di-substituted with substituents selected from oxo, alkyl, alkoxy, OH, halo and CF_{3;}
R₆ is selected from H, small alkyl, and —(CH₂)-phenyl; and R₃₅ is selected from H, small alkyl, and cyclopropyl.
R₂ can be selected from —C(═O)—N(R₄)(R₅),—CH₂—NH—C(═O) CH₃,—CH₂—NH—S(═O)₂(cyclopropyl),—CH₂—OH, and

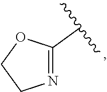

wherein:
R₄ is H or small alkyl, and
R₅ is selected from alkyl, OH, —S(═O)₂-(small alkyl), —(CH₂)₁₋₃—(C═O)—OH, —(CH₂)₀₋₃-cycloalkyl^{a}, —(CH₂)₀₋₃-heterocycloalkyl, —(CH(alkyl))-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, —(C(H)(heteroaryl))-alkyl, —(C(H)(alkyl))—(C═O)—OH, and —(C(H)(heteroaryl))—(C═O)—OR_{28;}
or R₄ and R₅ together with the nitrogen atom to which they are attached can form a carbon-containing 4-, 5-, or 6-membered saturated heterocyclic ring, optionally containing an additional heteroatom selected from N, and O, which may be optionally mono- or di-substituted with substituents selected from oxo, alkyl, alkoxy, OH, halo and CF_{3.}
R₂ can be selected from —C(═O)—N(R₄)(R₅),—CH₂—NH—S(═O)₂(cyclopropyl), and —CH₂—OH wherein: R₄ is H or small alkyl, and

R₅ is selected from alkyl, —(CH₂)₀₋₃-cycloalkyl, —(CH₂)₀₋₃-heterocycloalkyl, —(CH(alkyl)-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, and —(C(H)(heteroaryl))-alkyl;
or R₄ and R₅ together with the nitrogen atom to which they are attached can form a carbon-containing 5-, or 6-membered saturated heterocyclic ring, optionally containing an additional heteroatom selected from N, and O, which may be optionally mono- or di-substituted with substituents selected from oxo, alkyl, alkoxy, OH, halo and CF_{3.}
R₂ can be —C(═O)—N(R₄)(R₅), wherein: R₄ is H or small alkyl, and
R₅ is selected from alkyl, —(CH₂)₀₋₃-cycloalkyl, —(CH₂)₀₋₃-heterocycloalkyl, —(CH₂)₀₋₃-polycycloalkyl, —(CH₂)₀₋₃-aryl, —(C(H)(phenyl)-alkyl, —(CH₂)₀₋₃heteroaryl, and —(C(H)(heteroaryl)-alkyl;
or R₄ and R₅ together with the nitrogen atom to which they are attached can form a carbon-containing 5-, or 6-membered saturated heterocyclic ring, optionally containing an additional heteroatom selected from N, and O, which may be optionally mono- or di-substituted with substituents selected from oxo, alkyl, alkoxy, OH, halo and CF_{3.}
R₂ can be selected from —C(═O)—N(R₄)(R₅), —C(═O)—O—R₆,—CH₂—NH—C(═O) CH₃,—CH₂—NH—S(═O) z (cyclopropyl),

—CH₂—O—R₁₆,—CH₂—OH, —CN, and

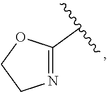

wherein:
R₄ is H or small alkyl, and
R₅ is selected from alkyl, —(CH₂)₀₋₃-cycloalkyl^{b}, —(CH₂)₀₋₃-heterocycloalkyl, —(CH(alkyl)-heterocycloalkyl, and —(CH₂)₀₋₃-polycycloalkyl; and
R₆ is selected from H and small alkyl; and R₁₆ is selected from H, small alkyl, and heterocycloalkyl.
R₂ can be —C(═O)—N(R₄)(R₅), wherein:
R₄ is H or small alkyl, and
R₅ is selected from small alkyl, pyrrolidine, piperidine, and tetrahydrofuran, wherein the pyrrolidine, piperidine, and tetrahydrofuran may optionally be substituted with 1 or 2 substituents independently selected from small alkyl, cyclopropyl, small alkoxy, OH, CN, CF₃, and halo.
R₂ can be selected from:

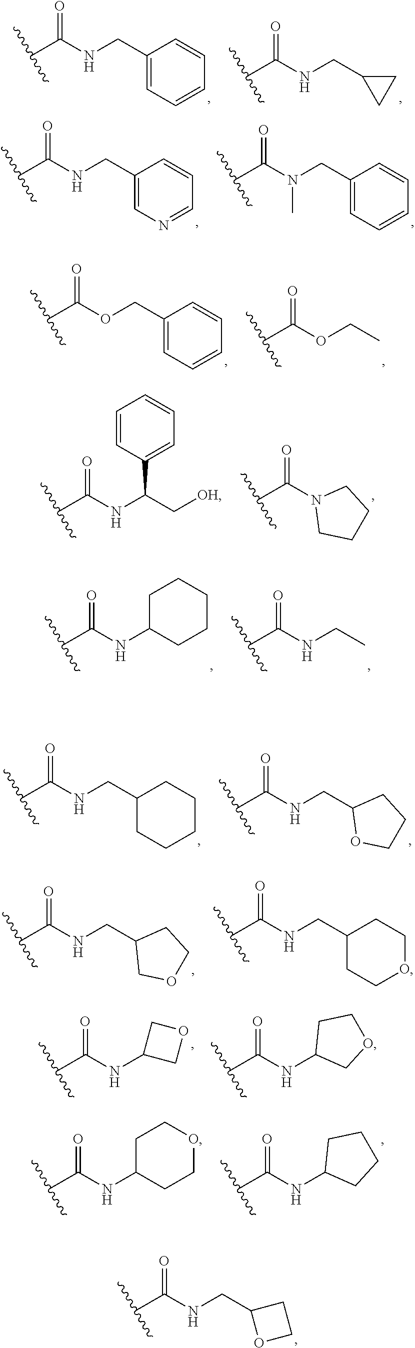

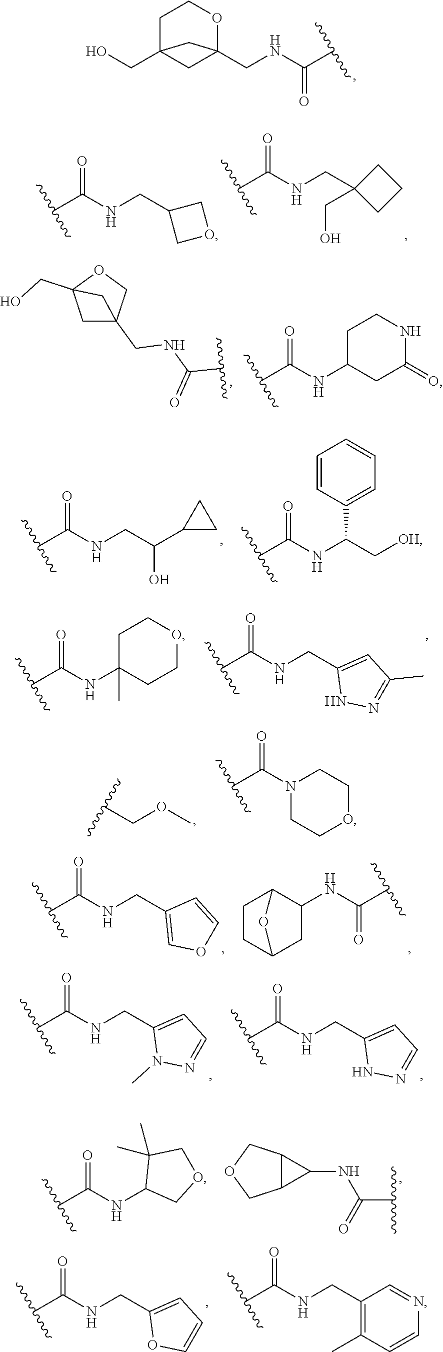

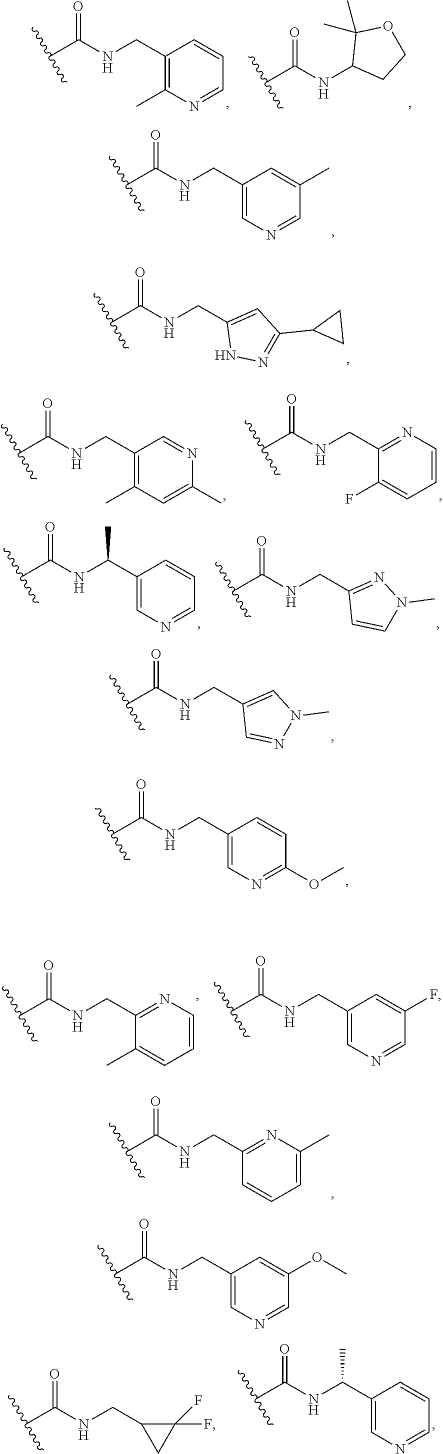

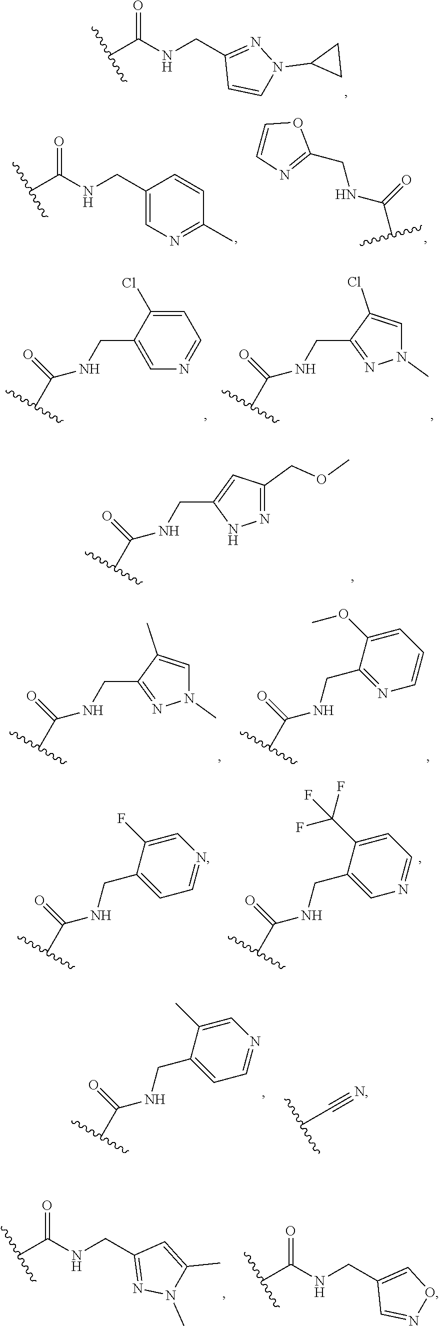

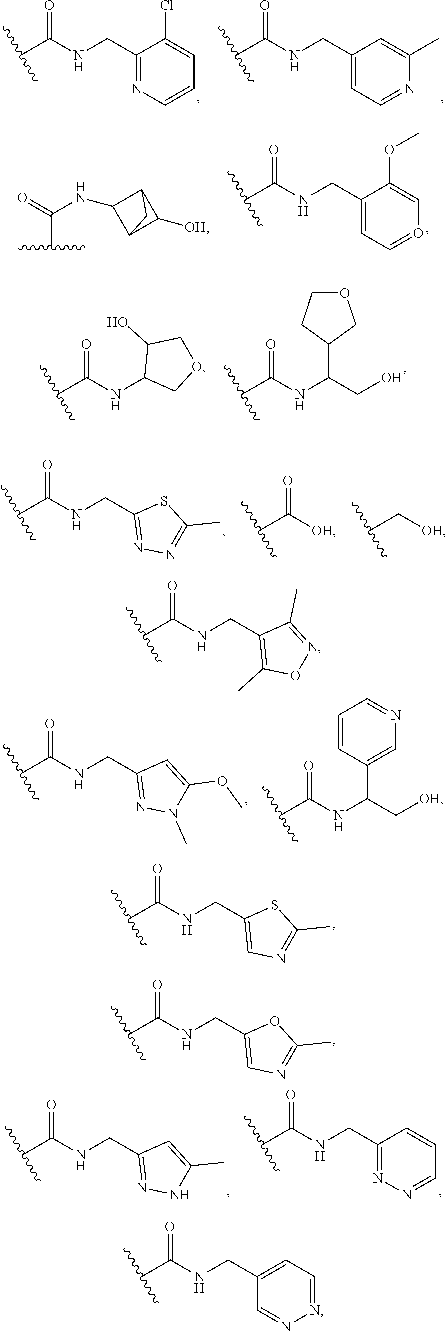

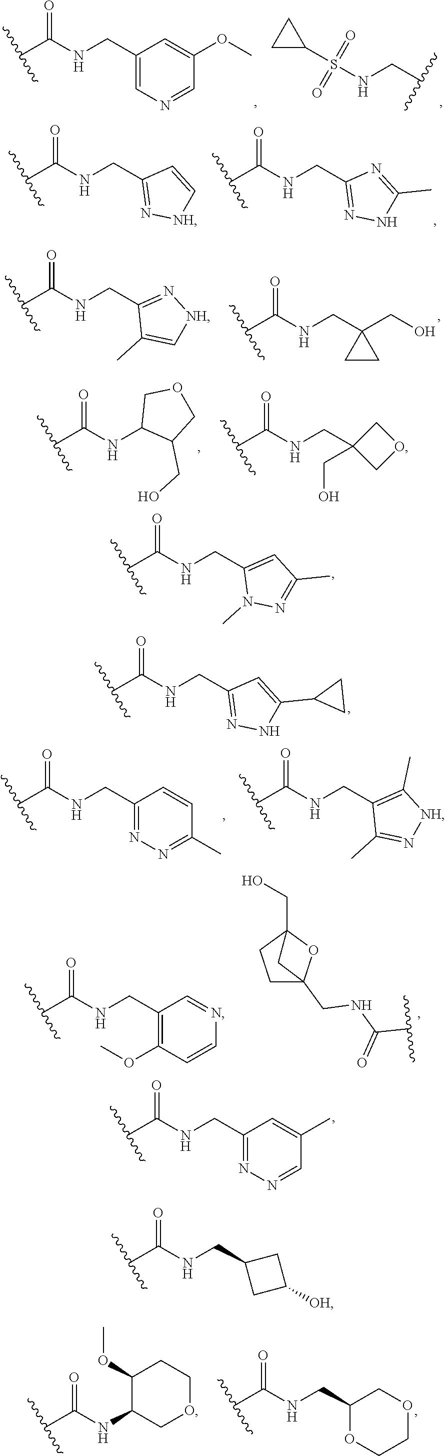

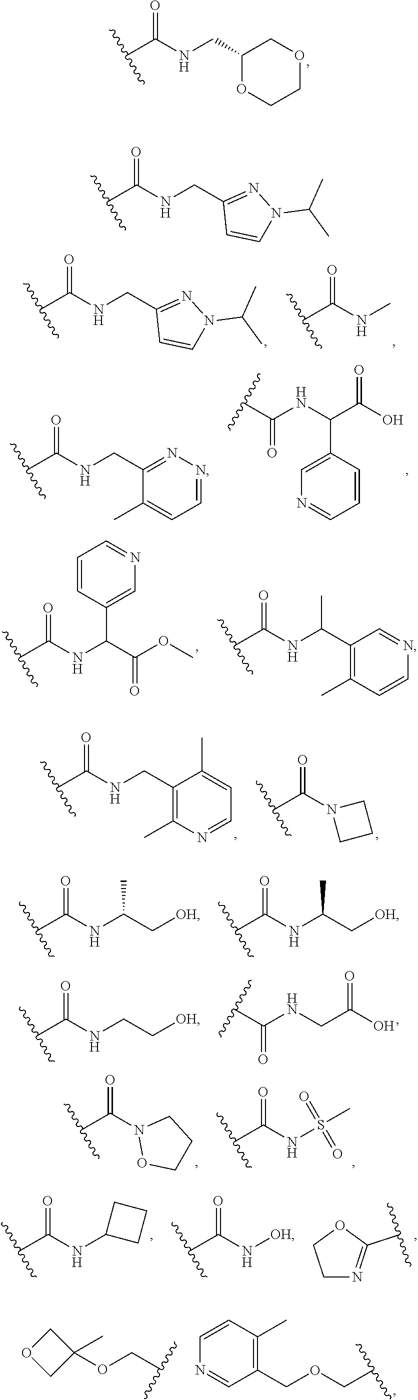

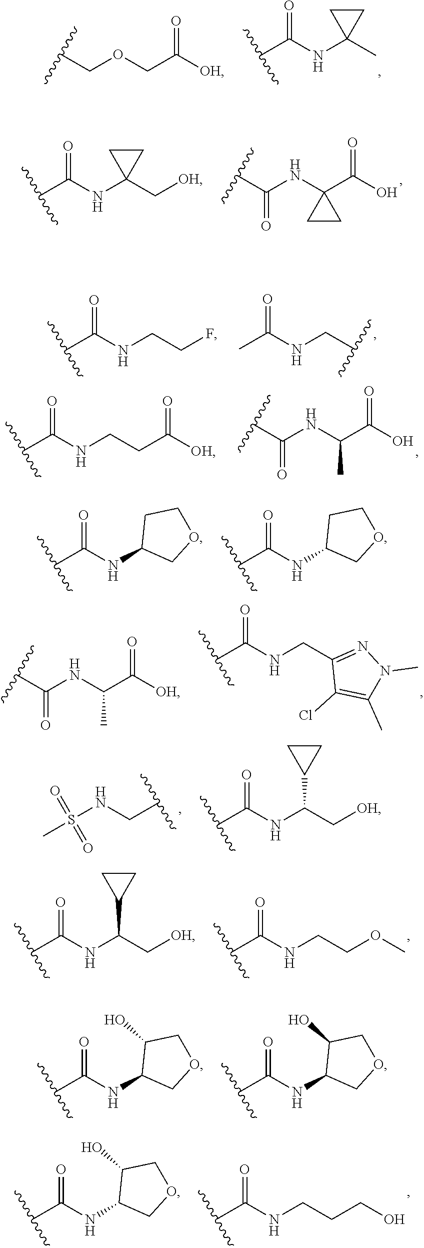

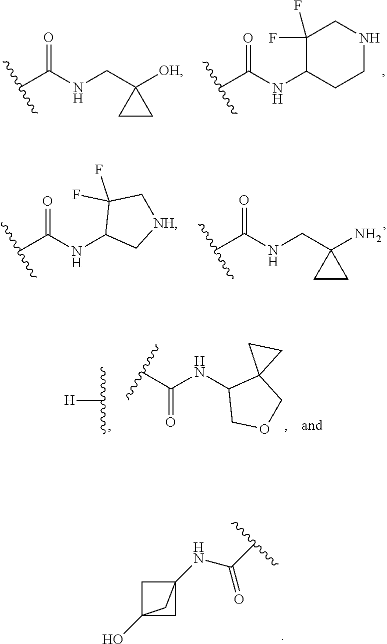

R₂ can be selected from:

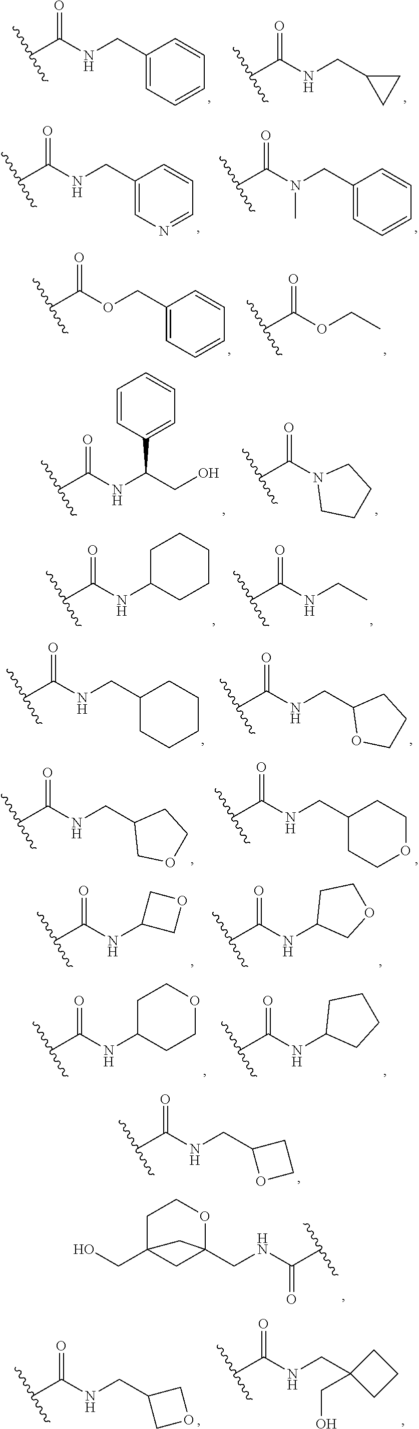

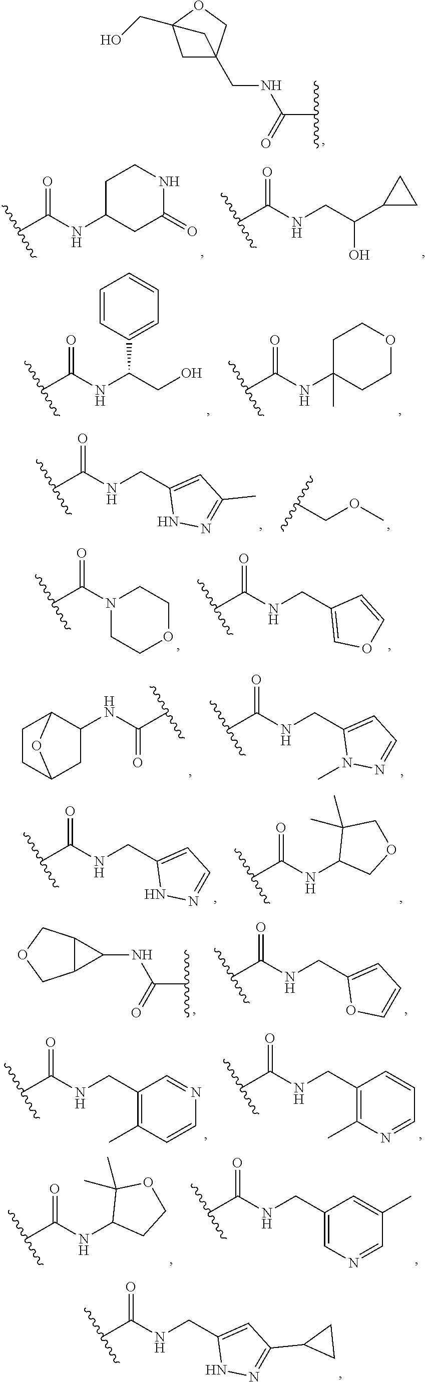

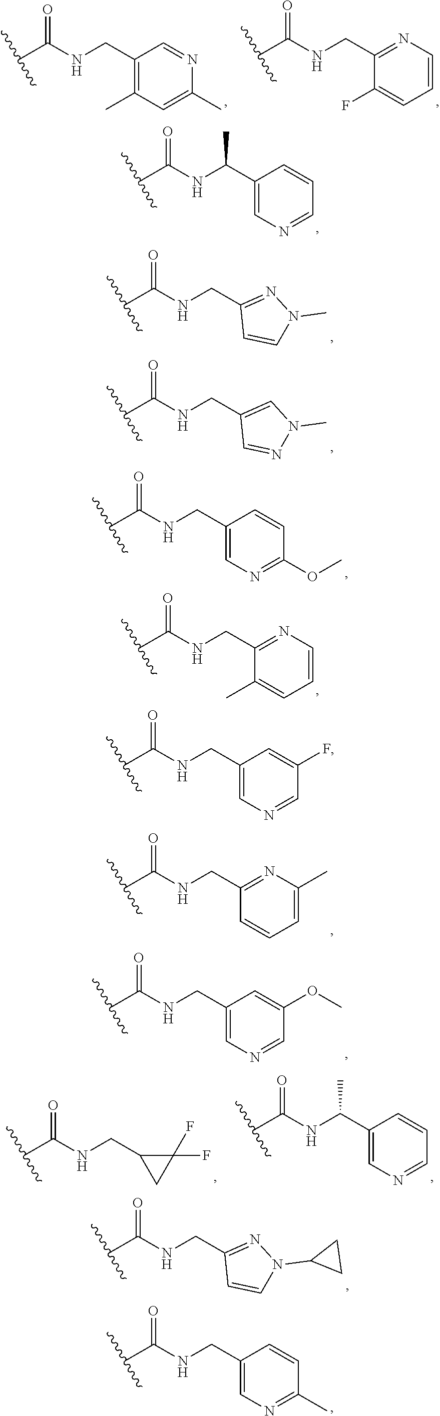

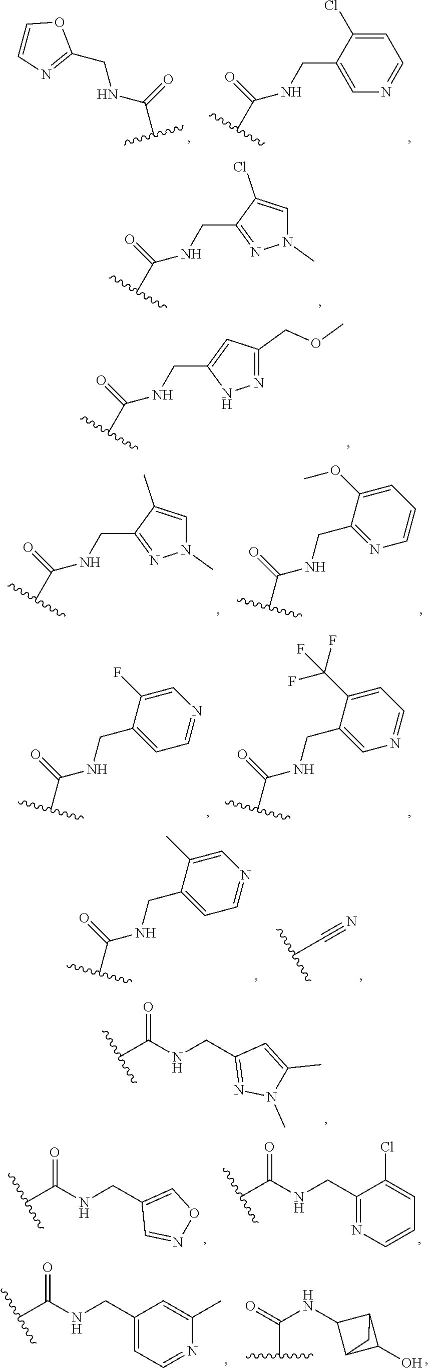

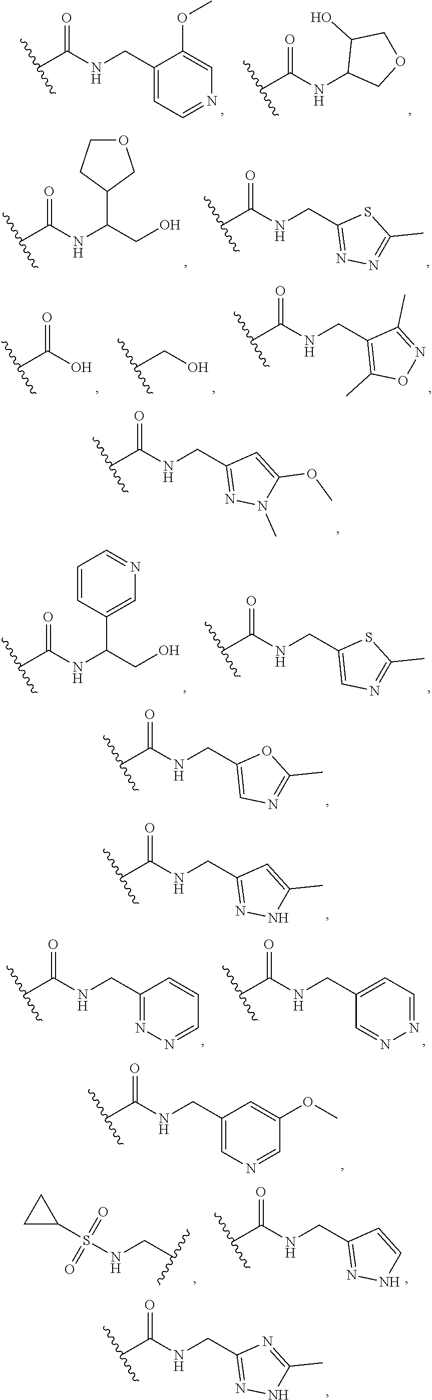

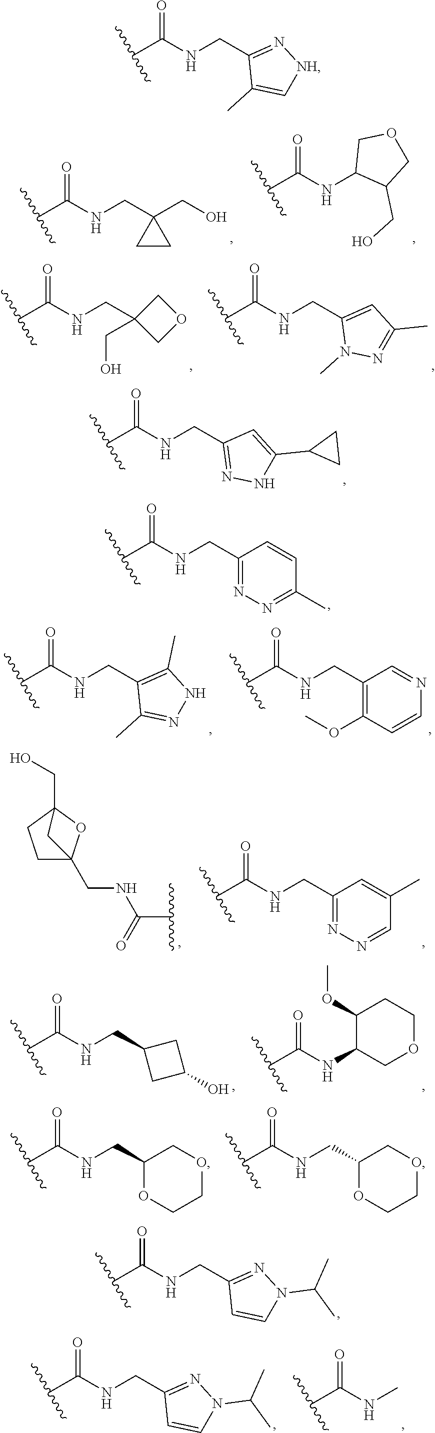

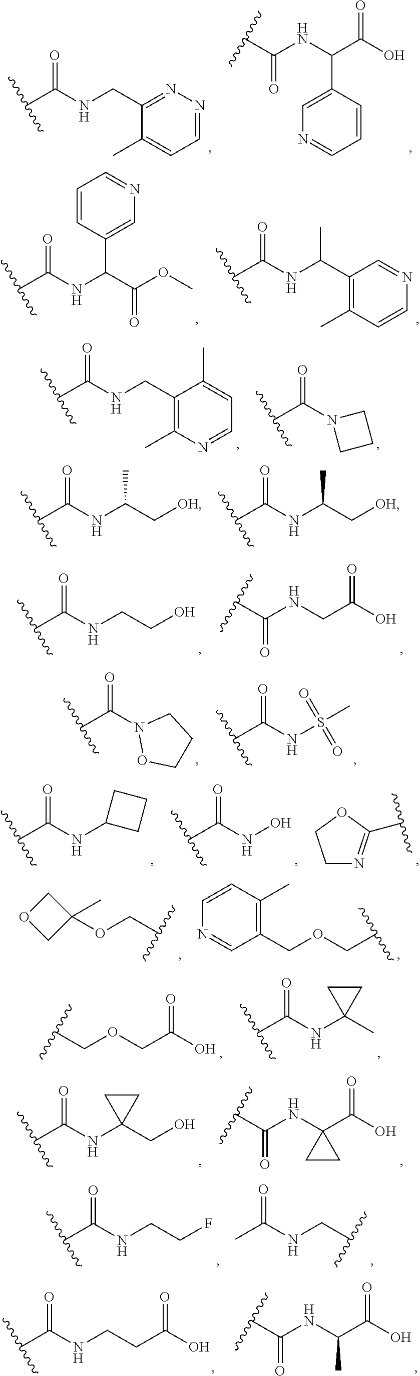

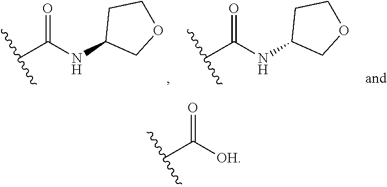

5 R₂ can be selected from:

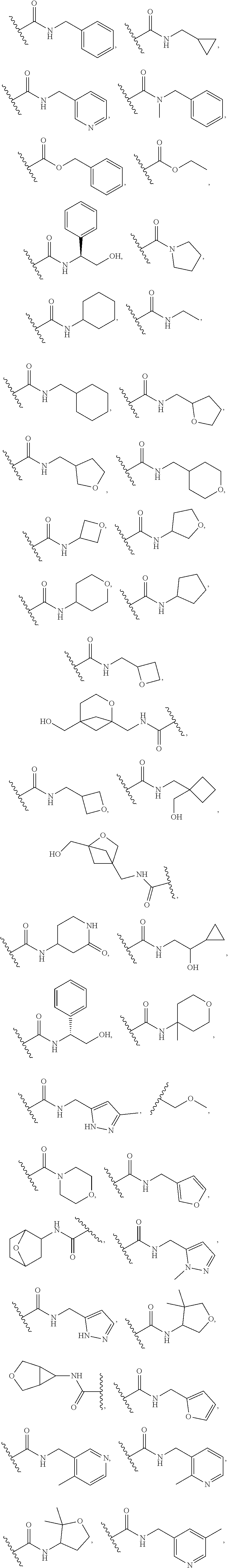

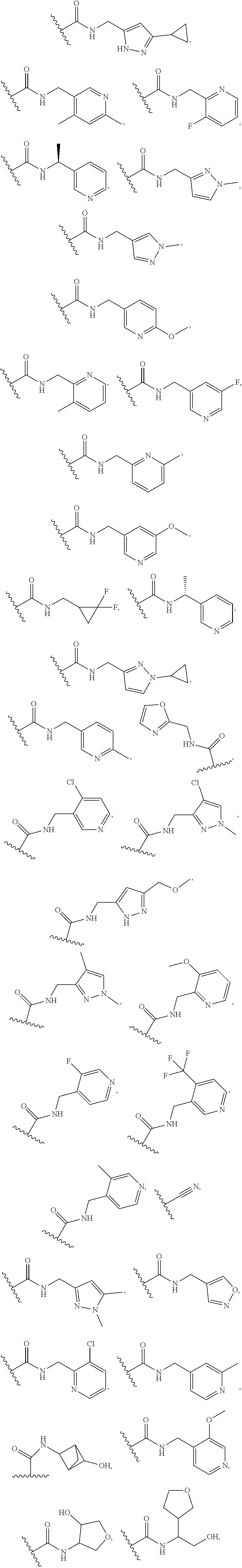

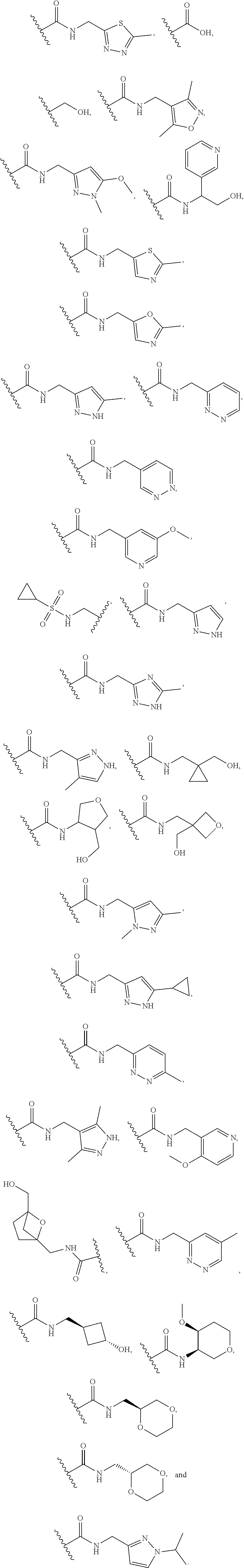

R₂ can be selected from:

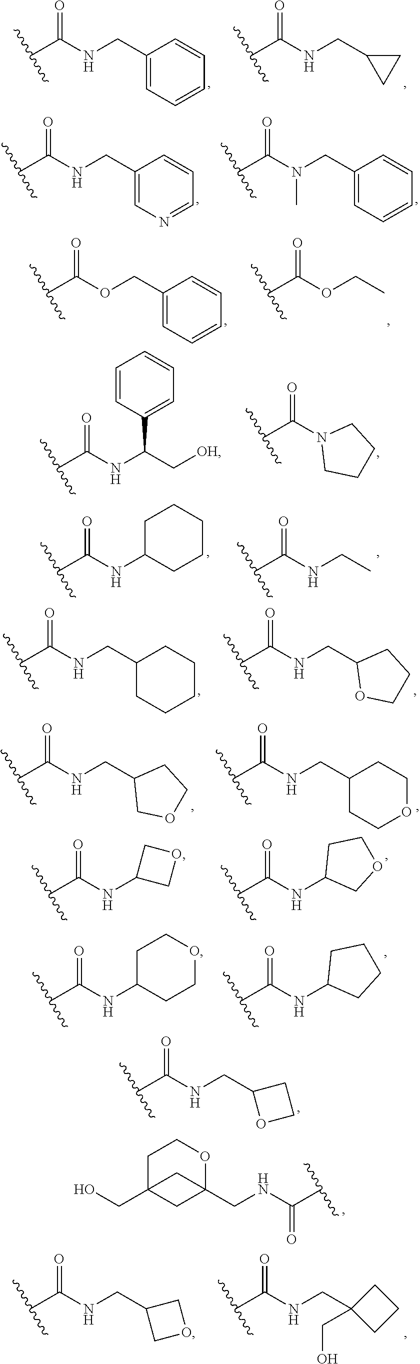

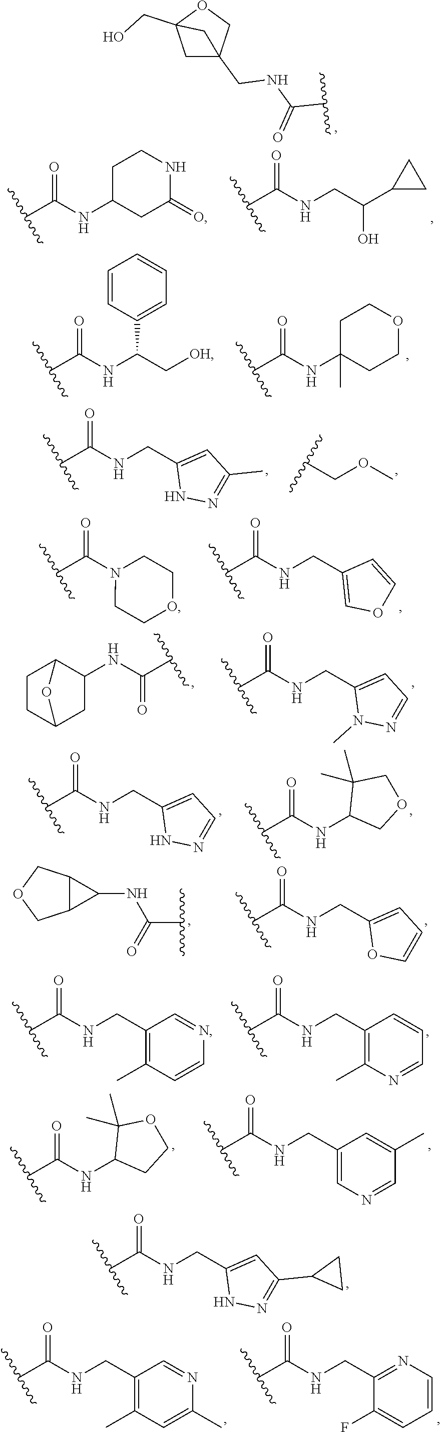

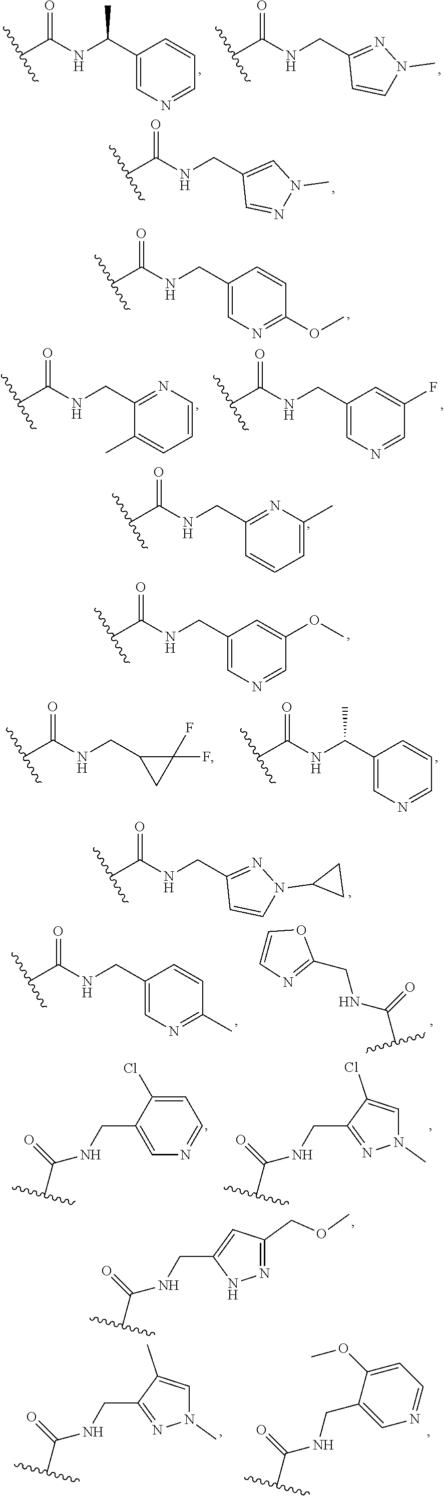

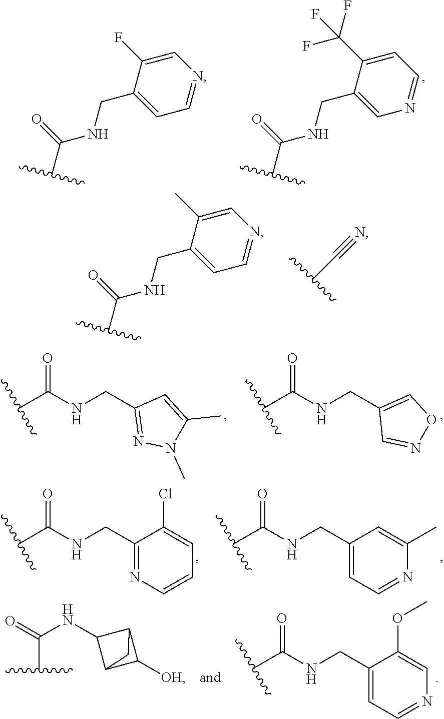

Preferably, R₂ can be selected from:

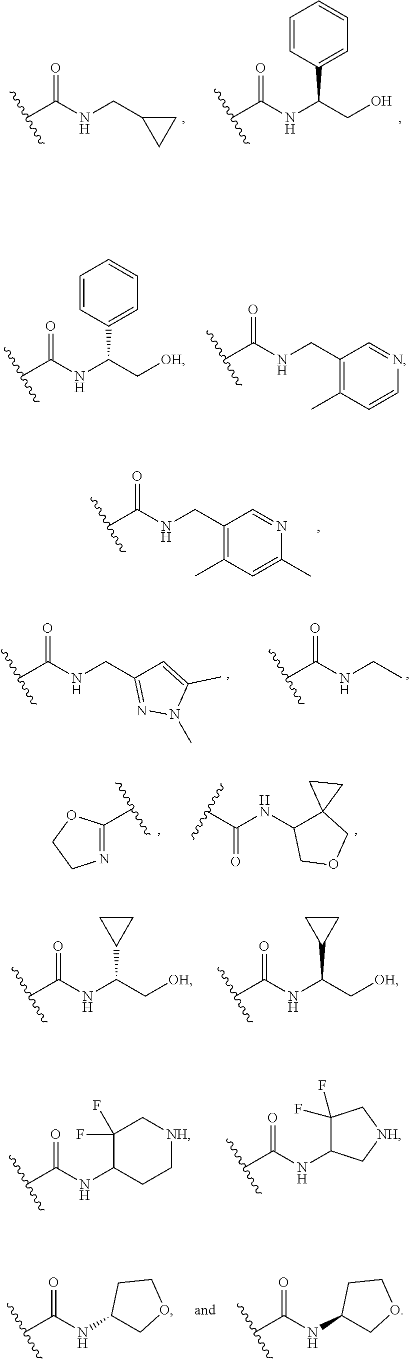

More preferably, R₂ is selected from:

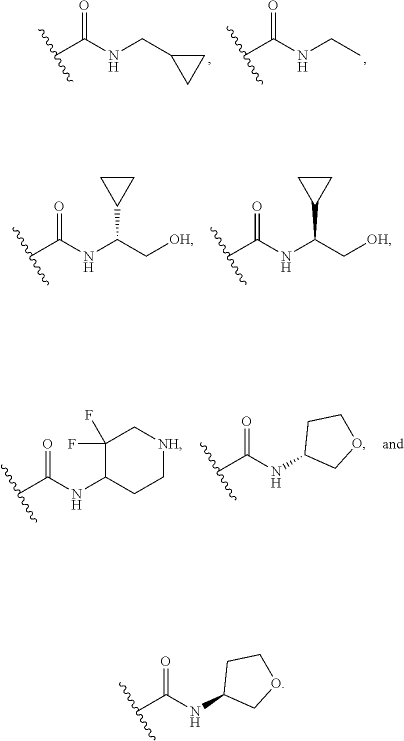

R₂ can be selected from:

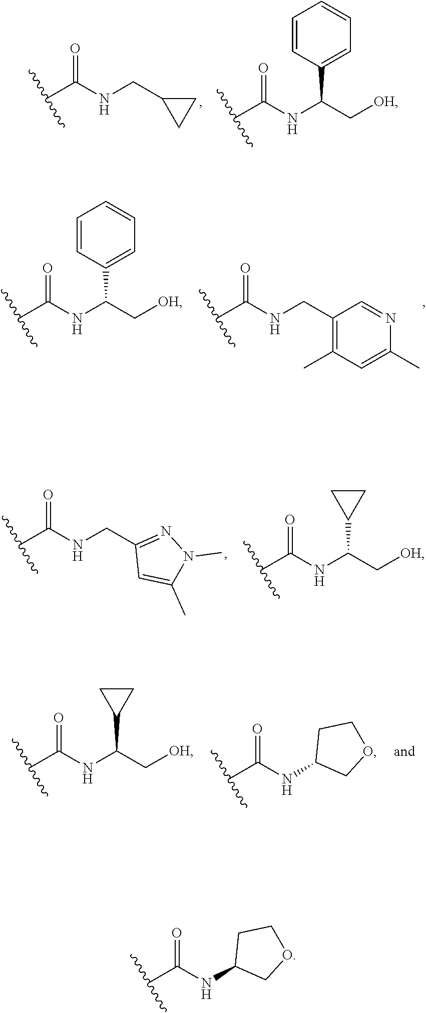

R₂ can be selected from:

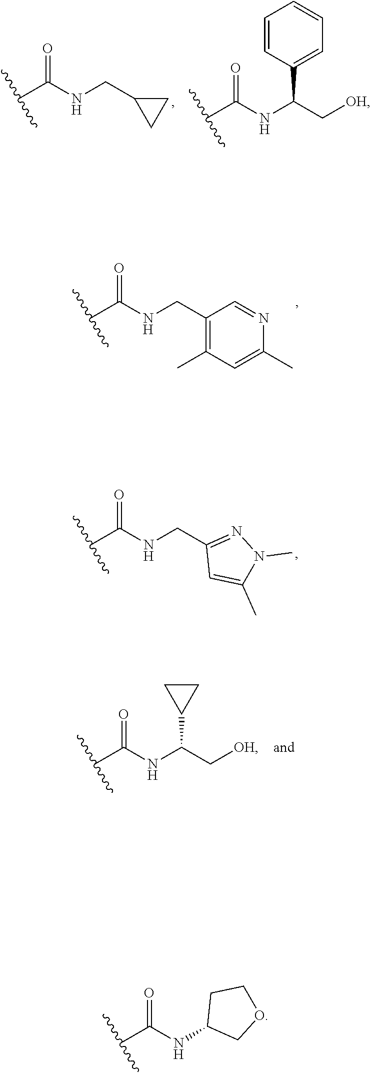

R₂ can be selected from:

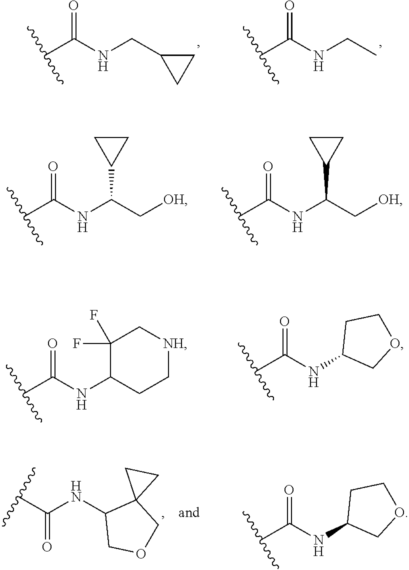

Even more preferably, R₂ is selected from:

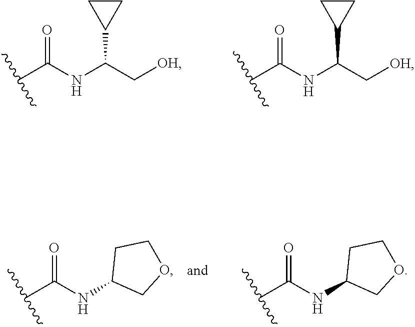 [SMILES: *C(=O)N[C@@H](CO)C1CC1 ; *C(=O)N[C@H](CO)C1CC1 ; *C(=O)N[C@@H]1CCOC1 ; *C(=O)N[C@H]1CCOC1]

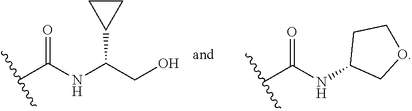 [SMILES: *C(=O)N[C@@H](CO)C1CC1 ; *C(=O)N[C@@H]1CCOC1]

Most preferably, R₂ is selected from:
R₂ can be selected from:

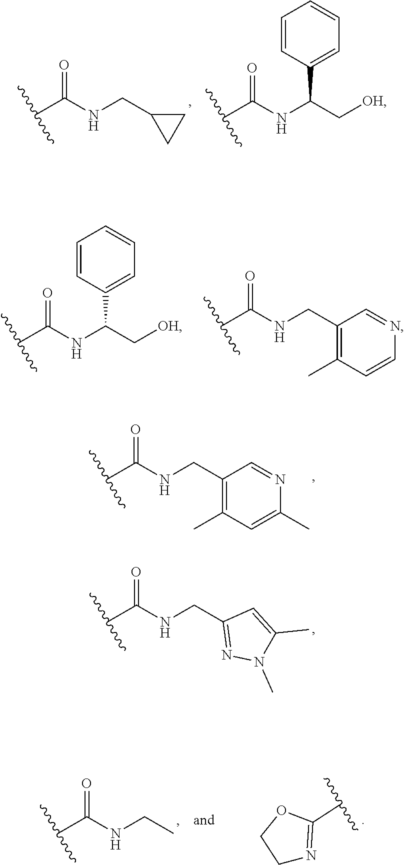

R₂ can be selected from:

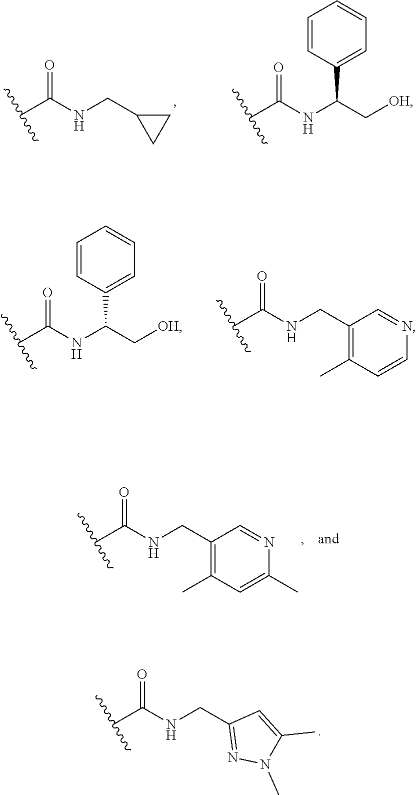 [SMILES: *C(=O)NCC1CC1 ; *C(=O)N[C@H](CO)c1ccccc1 ; *C(=O)N[C@@H](CO)c1ccccc1 ; *C(=O)NCc1cnccc1C ; *C(=O)NCc1cnc(C)cc1C ; *C(=O)NCc1cc(C)n(C)n1]

B₁ can be a carbon centre and B₂ can be a carbon centre, wherein B₃ is a single bond between B₂ and the nitrogen atom to which R₁ is attached and B₁ is connected to B₂ via a single bond, or B₁ can be a nitrogen centre and B₂ can be a carbon centre wherein B₁ is connected to B₂ via a double bond; and R₂ can be selected from —C(═O)—N(R₄)(R₅), —C(═O)—O—R₆,—CH₂—NH—C(═O) CH₃,—CH₂—NH—S(═O)₂R₃₅,

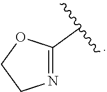 [SMILES: *C1=NCCO1]

—CH₂—O—R₁₆,—CH₂—OH, —CN, and
B₁ can be a carbon centre and B₂ can be a carbon centre, wherein B₃ is a single bond between B₂ and the nitrogen atom to which R₁ is attached and B₁ is connected to B₂ via a single bond, or B₁ can be a nitrogen centre and B₂ can be a carbon centre wherein B₁ is connected to B₂ via a double bond; and R₂ can be selected from —C(═O)—N(R₄)(R₅), —C(═O)—O—R₆,—CH₂—NH—C(═O) CH₃,—CH₂—NH—S(═O)₂(cyclopropyl),

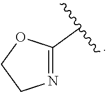 [SMILES: *C1=NCCO1]

—CH₂—O—R₁₆,—CH₂—OH, —CN, and
B₁ can be a carbon centre and B₂ can be a carbon centre, wherein B₃ is a single bond between B₂ and the nitrogen atom to which R₁ is attached and B₁ is connected to B₂ via a single bond, or B₁ can be a nitrogen centre and B₂ can be a carbon centre wherein B₁ is connected to B₂ via a double bond; and R₂ can be selected from —C(═O)—N(R₄)(R₅), —C(═O)—O—R₆,—CH₂—NH—S(═O)₂(cyclopropyl),—CH₂—O—CH₃,—CH₂—OH, and —CN.
B₁ can be a carbon centre and B₂ can be a carbon centre, wherein B₃ is a single bond between B₂ and the nitrogen atom to which R₁ is attached and B₁ is connected to B₂ via a single bond, and R₂ can be selected from —C(═O)—N(R₄)(R₅), —C(═O)—O—R₆,—CH₂—O—CH₃, and —CN.
B₄ can be N(R₂), B₅ can be N, and B₆ can be C(R₃), wherein B₄ is connected to B₅ via a single bond and B₅ is connected to B₆ via a double bond, or B₄ can be C(R₂), Bs can be N, and B₆ can be N(R₃), wherein B₄ is connected to B₅ via a double bond and B₅ is connected to B₆ via a single bond; and R₂ can be selected from —C(═O)—N(R₄)(R₅), —C(═O)—O—R₆,—CH₂—NH—C(═O) CH₃,—CH₂—NH—S(═O)₂R₃₅,—CH₂—O—R₁₆,—CH₂—OH, —CN, and

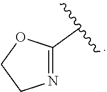 [SMILES: *C1=NCCO1]

B₄ can be N(R₂), Bs can be N, and B₆ can be C(R₃), wherein B₄ is connected to B₅ via a single bond and B₅ is connected to B₆ via a double bond, or B₄ can be C(R₂), Bs can be N, and B₆ can be N(R₃), wherein B₄ is connected to B₅ via a double bond and B₅ is connected to B₆ via a single bond; and R₂ can be selected from —C(═O)—N(R₄)(R₅), —C(═O)—O—R₆,—CH₂—NH—C(═O) CH₃,—CH₂—NH—S(═O)₂(cyclopropyl),—CH₂—O—R₁₆,—CH₂—OH, —CN,

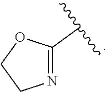 [SMILES: *C1=NCCO1]

and
B₄ can be N(R₂), B₅ can be N, and B₆ can be C(R₃), wherein B₄ is connected to B₅ via a single bond and B₅ is connected to B₆ via a double bond, or B₄ can be C(R₂), B₅ can be N, and B₆ can be N(R₃), wherein B₄ is connected to B₅ via a double bond and B₅ is connected to B₆ via a single bond; and R₂ can be selected from —C(═O)—N(R₄)(R₅), —C(═O)—O—R₆,—CH₂—NH—S(═O)₂(cyclopropyl),—CH₂—O—CH₃,—CH₂—OH, and —CN.
B₄ can be C(R₂), B₅ can be N, and B₆ can be N(R₃), wherein B₄ is connected to B₅ via a double bond and B₅ is connected to B₆ via a single bond, and R₂ can be selected from —C(═O)—N(R₄)(R₅), —C(═O)—O—R₆,—CH₂—O-CH₃, and —CN.
B₁ can be a carbon centre and B₂ can be a carbon centre, wherein B₃ is a single bond between B₂ and the nitrogen atom to which R₁ is attached and B₁ is connected to B₂ via a single bond, or B₁ can be a nitrogen centre and B₂ can be a carbon centre wherein B₁ is connected to B₂ via a double bond; B₄ can be N(R₂), B₅ can be N, and B₆ can be C(R₃), wherein B₄ is connected to B₅ via a single bond and B₅ is connected to B₆ via a double bond, or B₄ can be C(R₂), B₅ can be N, and B₆ can be N(R₃), wherein B₄ is connected to B₅ via a double bond and B₅ is connected to B₆ via a single bond; and R₂ can be selected from —C(═O)—N(R₄)(R₅),

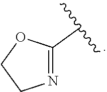 [SMILES: *C1=NCCO1]

—C(═O)—O—R₆,—CH₂—NH—S(═O)₂R₃₅,—CH₂—NH—C(═O) CH₃,—CH₂—O—R₁₆,—CH₂—OH, —CN, and
B₁ can be a carbon centre and B₂ can be a carbon centre, wherein B₃ is a single bond between B₂ and the nitrogen atom to which R₁ is attached and B₁ is connected to B₂ via a single bond, or B₁ can be a nitrogen centre and B₂ can be a carbon centre wherein B₁ is connected to B₂ via a double bond; B₄ can be N(R₂), B₅ can be N, and B₆ can be C(R₃), wherein B₄ is connected to B₅ via a single bond and B₅ is connected to B₆ via a double bond, or B₄ can be C(R₂), B₅ can be N, and B₆ can be N(R₃), wherein B₄ is connected to B₅ via a double bond and B₅ is connected to B₆ via a single bond; and R₂ can be selected from —C(═O)—N(R₄)(R₅),

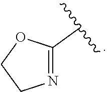 [SMILES: *C1=NCCO1]

—C(═O)—O—R₆,—CH₂—NH—S(═O)₂(cyclopropyl),—CH₂—NH—C(═O) CH₃,—CH₂—O—R₁₆,—CH₂—OH, —CN, and
B₁ can be a carbon centre and B₂ can be a carbon centre, wherein B₃ is a single bond between B₂ and the nitrogen atom to which R₁ is attached and B₁ is connected to B₂ via a single bond, or B₁ can be a nitrogen centre and B₂ can be a carbon centre wherein B₁ is connected to B₂ via a double bond; B₄ can be N(R₂), B₅ can be N, and B₆ can be C(R₃), wherein B₄ is connected to B₅ via a single bond and B₅ is connected to B₆ via a double bond, or B₄ can be C(R₂), B₅ can be N, and B₆ can be N(R₃), wherein B₄ is connected to B₅ via a double bond and B₅ is connected to B₆ via a single bond; and R₂ can be selected from —C(═O)—N(R₄)(R₅), —C(═O)—O—R₆,—CH₂—NH—S(═O)₂(cyclopropyl),—CH₂—O—CH₃,—CH₂—OH, and —CN.
B₁ can be a carbon centre and B₂ can be a carbon centre, wherein B₃ is a single bond between B₂ and the nitrogen atom to which R₁ is attached and B₁ is connected to B₂ via a single bond; B₄ can be C(R₂), B₅ can be N, and B₆ can be N(R₃), wherein B₄ is connected to B₅ via a double bond and B₅ is connected to B₆ via a single bond; and R₂ can be selected from —C(═O)—N(R₄)(R₅), —C(═O)—O—R₆,—CH₂—O—CH₃, and —CN.
B₁ can be a carbon centre and B₂ can be a carbon centre, wherein B₃ is a single bond between B₂ and the nitrogen atom to which R₁ is attached and B₁ is connected to B₂ via a single bond, or B₁ can be a nitrogen centre and B₂ can be a carbon centre wherein B₁ is connected to B₂ via a double bond; and R₂ can be selected from —C(═O)—N(R₄)(R₅), —C(═O)—O—R₆,—CH₂—NH—C(═O) CH₃,—CH₂—NH—S(═O)₂R₃₅,—CH₂—O—R₁₆,—CH₂—OH, —

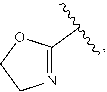 [SMILES: *C1=NCCO1]

CN, and wherein:
R₄ is H or small alkyl, and
R₅ is selected from alkyl, OH, —S(═O)₂-(small alkyl), —(CH₂)₁₋₃—(C═O)—OH, —(CH₂)₀₋₃-cycloalkyl^{b}, —(CH₂)₀₋₃-heterocycloalkyl, —(CH(alkyl))-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, —(C(H)(heteroaryl))-alkyl, —(C(H)(alkyl))—(C═O)—OH, and —(C(H)(heteroaryl))—(C═O)—OR_{28;}
or R₄ and R₅ together with the nitrogen atom to which they are attached can form a carbon-containing 4-, 5-, or 6-membered saturated heterocyclic ring, optionally containing an additional heteroatom selected from N, and O, which may be optionally mono- or di-substituted with substituents selected from oxo, alkyl, alkoxy, OH, halo and CF_{3;}
R₆ is H, small alkyl, and —(CH₂)-phenyl;
R₁₆ is selected from H, small alkyl, —(CH₂)—(C═O)—OH, heterocycloalkyl, and —(CH₂)-heteroaryl; and R₃₅ is selected from H, small alkyl, and cyclopropyl.
B₁ can be a carbon centre and B₂ can be a carbon centre, wherein B₃ is a single bond between B₂ and the nitrogen atom to which R₁ is attached and B₁ is connected to B₂ via a single bond, or B₁ can be a nitrogen centre and B₂ can be a carbon centre wherein B₁ is connected to B₂ via a double bond; and R₂ can be selected from —C(═O)—N(R₄)(R₅), —C(═O)—O—R₆,—CH₂—NH—C(═O) CH₃,—CH₂—NH—S(═O)₂(cyclopropyl),—CH₂—O—R₁₆,

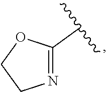 [SMILES: *C1=NCCO1]

—CH₂—OH, —CN, and wherein:
R₄ is H or small alkyl, and
R₅ is selected from alkyl, OH, —S(═O)₂-(small alkyl), —(CH₂)₁₋₃—(C═O)—OH, —(CH₂)₀₋₃-cycloalkyl^{a}, —(CH₂)₀₋₃-heterocycloalkyl, —(CH(alkyl))-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, —(C(H)(heteroaryl))-alkyl, —(C(H)(alkyl))—(C═O)—OH, and —(C(H)(heteroaryl))—(C═O)—OR_{28;}
or R₄ and R₅ together with the nitrogen atom to which they are attached can form a carbon-containing 4-, 5-, or 6-membered saturated heterocyclic ring, optionally containing an additional heteroatom selected from N, and O, which may be optionally mono- or di-substituted with substituents selected from oxo, alkyl, alkoxy, OH, halo and CF₃; and R₆ is H, small alkyl, and —(CH₂)-phenyl.
B₁ can be a carbon centre and B₂ can be a carbon centre, wherein B₃ is a single bond between B₂ and the nitrogen atom to which R₁ is attached and B₁ is connected to B₂ via a single bond, or B₁ can be a nitrogen centre and B₂ can be a carbon centre wherein B₁ is connected to B₂ via a double bond; and R₂ can be selected from —C(═O)—N(R₄)(R₅), —C(═O)—O—R₆,—CH₂—NH—S(═O)₂(cyclopropyl),—CH₂—O—CH₃,—CH₂—OH, and —CN, wherein:
R₄ is H or small alkyl, and
R₅ is selected from alkyl, —(CH₂)₀₋₃-cycloalkyl, —(CH₂)₀₋₃-heterocycloalkyl, —(CH(alkyl)-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, and —(C(H)(heteroaryl))-alkyl;
or R₄ and R₅ together with the nitrogen atom to which they are attached can form a carbon-containing 5-, or 6-membered saturated heterocyclic ring, optionally containing an additional heteroatom selected from N, and O, which may be optionally mono- or di-substituted with substituents selected from oxo, alkyl, alkoxy, OH, halo and CF₃; and R₆ is H, small alkyl, and —(CH₂)-phenyl.
B₁ can be a carbon centre and B₂ can be a carbon centre, wherein B₃ is a single bond between B₂ and the nitrogen atom to which R₁ is attached and B₁ is connected to B₂ via a single bond, and R₂ can be selected from —C(═O)—N(R₄)(R₅), —C(═O)—O—R₆,—CH₂—O—CH₃, and —CN, wherein: R₄ is H or small alkyl, and selected —(CH₂)₀₋₃-heterocycloalkyl, alkyl, —(CH₂)₀₋₃-cycloalkyl, —(CH₂)₀₋₃-polycycloalkyl, —(C(H)(phenyl))-alkyl, —(CH₂)₀₋₃heteroaryl, and —(C(H)(heteroaryl))-alkyl;
R₅ is from —(CH₂)₀₋₃-aryl,
or R₄ and R₅ together with the nitrogen atom to which they are attached can form a carbon-containing 5-, or 6-membered saturated heterocyclic ring, optionally containing an additional heteroatom selected from N, and O, which may be optionally mono- or di-substituted with substituents selected from oxo, alkyl, alkoxy, OH, halo and CF₃; and R₆ is H, small alkyl, and —(CH₂)-phenyl.
B₄ can be N(R₂), B₅ can be N, and B₆ can be C(R₃), wherein B₄ is connected to B₅ via a single bond and B₅ is connected to B₆ via a double bond, or B₄ can be C(R₂), B₅ can be N, and B₆ can be N(R₃), wherein B₄ is connected to B₅ via a double bond and B₅ is connected to B₆ via a single bond; and R₂ can be selected from —C(═O)—N(R₄)(R₅), —C(═O)—O—R₆,—CH₂—NH—C(═O) CH₃,—CH₂—NH—S(═O)₂R₃₅,—CH₂—O—R₁₆,—CH₂—OH, —CN, and

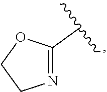

wherein:
R₄ is H or small alkyl, and
R₅ is selected from alkyl, OH, —S(═O)₂-(small alkyl), —(CH₂)₁₋₃—(C═O)—OH, —(CH₂)₀₋₃-cycloalkyl^{b}, —(CH₂)₀₋₃-heterocycloalkyl, —(CH(alkyl))-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, —(C(H)(heteroaryl))-alkyl, —(C(H)(alkyl))—(C═O)—OH, and —(C(H)(heteroaryl))—(C═O)—OR_{28;}
or R₄ and R₅ together with the nitrogen atom to which they are attached can form a carbon-containing 4-, 5-, or 6-membered saturated heterocyclic ring, optionally containing an additional heteroatom selected from N, and O, which may be optionally mono- or di-substituted with substituents selected from oxo, alkyl, alkoxy, OH, halo and CF₃;
R₆ is H, small alkyl, and —(CH₂)-phenyl;
R₁₆ is selected from H, small alkyl, —(CH₂)—(C═O)—OH, heterocycloalkyl, and —(CH₂)-heteroaryl; and R₃₅ is selected from H, small alkyl, and cyclopropyl.
B₄ can be N(R₂), B₅ can be N, and B₆ can be C(R₃), wherein B₄ is connected to B₅ via a single bond and B₅ is connected to B₆ via a double bond, or B₄ can be C(R₂), B₅ can be N, and B₆ can be N(R₃), wherein B₄ is connected to B₅ via a double bond and B₅ is connected to B₆ via a single bond; and R₂ can be selected from —C(═O)—N(R₄)(R₅), —C(═O)—O—R₆,—CH₂—NH—C(═O) CH₃,—CH₂—NH—S(═O)₂(cyclopropyl),—CH₂—O—R₁₆,—CH₂—OH, —CN,

and

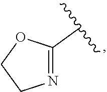

wherein:
R₄ is H or small alkyl, and
R₅ is selected from alkyl, OH, —S(═O)₂-(small alkyl), —(CH₂)₁₋₃—(C═O)—OH, —(CH₂)₀₋₃-cycloalkyl^{a}, —(CH₂)₀₋₃-heterocycloalkyl, —(CH(alkyl))-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, —(C(H)(heteroaryl))-alkyl, —(C(H)(alkyl))—(C═O)—OH, and —(C(H)(heteroaryl))—(C═O)—OR_{28;}
or R₄ and R₅ together with the nitrogen atom to which they are attached can form a carbon-containing 4-, 5-, or 6-membered saturated heterocyclic ring, optionally containing an additional heteroatom selected from N, and O, which may be optionally mono- or di-substituted with substituents selected from oxo, alkyl, alkoxy, OH, halo and CF₃; and
R₆ is H, small alkyl, and —(CH₂)-phenyl.
B₄ can be N(R₂), B₅ can be N, and B₆ can be C(R₃), wherein B₄ is connected to B₅ via a single bond and B₅ is connected to B₆ via a double bond, or B₄ can be C(R₂), B₅ can be N, and B₆ can be N(R₃), wherein B₄ is connected to B₅ via a double bond and B₅ is connected to B₆ via a single bond; and R₂ can be selected from —C(═O)—N(R₄)(R₅), —C(═O)—O—R₆,—CH₂—NH—S(═O)₂(cyclopropyl),—CH₂—O—CH₃,—CH₂—OH, and —CN, wherein:
R₄ is H or small alkyl, and
R₅ is selected from alkyl, —(CH₂)₀₋₃-cycloalkyl, —(CH₂)₀₋₃-heterocycloalkyl, —(CH(alkyl))-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, and —(C(H)(heteroaryl))-alkyl;
or R₄ and R₅ together with the nitrogen atom to which they are attached can form a carbon-containing 5-, or 6-membered saturated heterocyclic ring, optionally containing an additional heteroatom selected from N, and O, which may be optionally mono- or di-substituted with substituents selected from oxo, alkyl, alkoxy, OH, halo and CF₃; and
R₆ is H, small alkyl, and —(CH₂)-phenyl.
B₄ can be C(R₂), B₅ can be N, and B₆ can be N(R₃), wherein B₄ is connected to B₅ via a double bond and B₅ is connected to B₆ via a single bond, and R₂ can be selected from —C(═O)—N(R₄)(R₅), —C(═O)—O—R₆,—CH₂—O-CH₃, and —CN, wherein:
R₄ is H or small alkyl, and
R₅ is selected from alkyl, —(CH₂)₀₋₃-cycloalkyl, —(CH₂)₀₋₃-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, and —(C(H)(heteroaryl))-alkyl;
or R₄ and R₅ together with the nitrogen atom to which they are attached can form a carbon-containing 5-, or 6-membered saturated heterocyclic ring, optionally containing an additional heteroatom selected from N, and O, which may be optionally mono- or di-substituted with substituents selected from oxo, alkyl, alkoxy, OH, halo and CF₃; and R₆ is H, small alkyl, and —(CH₂)-phenyl.
B₁ can be a carbon centre and B₂ can be a carbon centre, wherein B₃ is a single bond between B₂ and the nitrogen atom to which R₁ is attached and B₁ is connected to B₂ via a single bond, or B₁ can be a nitrogen centre and B₂ can be a carbon centre wherein B₁ is connected to B₂ via a double bond; B₄ can be N(R₂), B₅ can be N, and B₆ can be C(R₃), wherein B₄ is connected to B₅ via a single bond and B₅ is connected to B₆ via a double bond, or B₄ can be C(R₂), B₅ can be N, and B₆ can be N(R₃), wherein B₄ is connected to B₅ via a double bond and B₅ is connected to B₆ via a single bond; and R₂ can be selected from —C(═O)—N(R₄)(R₅),

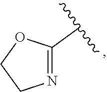 [SMILES: *C1=NCCO1]

—C(═O)—O—R₆,—CH₂—NH—C(═O) CH₃,—CH₂—NH—S(═O)₂R₃₅,—CH₂—O—R₁₆,—CH₂—OH, —CN, and R₄ is H or small alkyl, and wherein:
R₅ is selected from alkyl, OH, —S(═O)₂-(small alkyl), —(CH₂)₁₋₃—(C═O)—OH, —(CH₂)₀₋₃-cycloalkyl^{b}, —(CH₂)₀₋₃-heterocycloalkyl, —(CH(alkyl))-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, —(C(H)(heteroaryl))-alkyl, —(C(H)(alkyl))—(C═O)—OH, and —(C(H)(heteroaryl))—(C═O)—OR_{28;}
or R₄ and R₅ together with the nitrogen atom to which they are attached can form a carbon-containing 4-, 5-, or 6-membered saturated heterocyclic ring, optionally containing an additional heteroatom selected from N, and O, which may be optionally mono- or di-substituted with substituents selected from oxo, alkyl, alkoxy, OH, halo and CF₃; R₆ is H, small alkyl, and —(CH₂)-phenyl;
R₁₆ is selected from H, small alkyl, —(CH₂)—(C═O)—OH, heterocycloalkyl, and —(CH₂)-heteroaryl; and R₃₅ is selected from H, small alkyl, and cyclopropyl.
B₁ can be a carbon centre and B₂ can be a carbon centre, wherein B₃ is a single bond between B₂ and the nitrogen atom to which R₁ is attached and B₁ is connected to B₂ via a single bond, or B₁ can be a nitrogen centre and B₂ can be a carbon centre wherein B₁ is connected to B₂ via a double bond; B₄ can be N(R₂), B₅ can be N, and B₆ can be C(R₃), wherein B₄ is connected to B₅ via a single bond and B₅ is connected to B₆ via a double bond, or B₄ can be C(R₂), B₅ can be N, and B₆ can be N(R₃), wherein B₄ is connected to B₅ via a double bond and B₅ is connected to B₆ via a single bond; and R₂ can be selected from —C(═O)—N(R₄)(R₅),

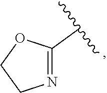 [SMILES: *C1=NCCO1]

—C(═O)—O—R₆,—CH₂—NH—C(═O) CH₃,—CH₂—NH—S(═O)₂(cyclopropyl),—CH₂—O—R₁₆,—CH₂—OH, —CN, and wherein:
R₄ is H or small alkyl, and
R₅ is selected from alkyl, OH, —S(═O)₂-(small alkyl), —(CH₂)₁₋₃—(C═O)—OH, —(CH₂)₀₋₃-cycloalkyl^{a}, —(CH₂)₀₋₃-heterocycloalkyl, —(CH(alkyl))-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, —(C(H)(heteroaryl))-alkyl, —(C(H)(alkyl))—(C═O)—OH, and —(C(H)(heteroaryl))—(C═O)—OR_{28;}
or R₄ and R₅ together with the nitrogen atom to which they are attached can form a carbon-containing 4-, 5-, or 6-membered saturated heterocyclic ring, optionally containing an additional heteroatom selected from N, and O, which may be optionally mono- or di-substituted with substituents selected from oxo, alkyl, alkoxy, OH, halo and CF₃; and
R₆ is H, small alkyl, and —(CH₂)-phenyl.
B₁ can be a carbon centre and B₂ can be a carbon centre, wherein B₃ is a single bond between B₂ and the nitrogen atom to which R₁ is attached and B₁ is connected to B₂ via a single bond, or B₁ can be a nitrogen centre and B₂ can be a carbon centre wherein B₁ is connected to B₂ via a double bond; B₄ can be N(R₂), B₅ can be N, and B₆ can be C(R₃), wherein B₄ is connected to B₅ via a single bond and B₅ is connected to B₆ via a double bond, or B₄ can be C(R₂), B₅ can be N, and B₆ can be N(R₃), wherein B₄ is connected to B₅ via a double bond and B₅ is connected to B₆ via a single bond; and R₂ can be selected from —C(═O)—N(R₄)(R₅), —C(═O)—O—R₆,—CH₂—NH—S(═O)₂(cyclopropyl),—CH₂—O—CH₃,—CH₂—OH, and —CN, wherein:
R₄ is H or small alkyl, and
R₅ is selected from alkyl, —(CH₂)₀₋₃-cycloalkyl, —(CH₂)₀₋₃-heterocycloalkyl, —(CH(alkyl))-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, and —(C(H)(heteroaryl))-alkyl;
or R₄ and R₅ together with the nitrogen atom to which they are attached can form a carbon-containing 5-, or 6-membered saturated heterocyclic ring, optionally containing an additional heteroatom selected from N, and O, which may be optionally mono- or di-substituted with substituents selected from oxo, alkyl, alkoxy, OH, halo and CF₃; and R₆ is H, small alkyl, and —(CH₂)-phenyl.
B₁ can be a carbon centre and B₂ can be a carbon centre, wherein B₃ is a single bond between B₂ and the nitrogen atom to which R₁ is attached and B₁ is connected to B₂ via a single bond; B₄ can be C(R₂), B₅ can be N, and B₆ can be N(R₃), wherein B₄ is connected to B₅ via a double bond and B₅ is connected to B₆ via a single bond; and R₂ can be selected from —C(═O)—N(R₄)(R₅), —C(═O)—O—R₆,—CH₂—O—CH₃, and —CN, wherein: R₄ is H or small alkyl, and
R₅ alkyl, —(CH₂)₀₋₃-cycloalkyl, —(CH₂)₀₋₃-heterocycloalkyl, —(CH₂)₀₋₃-polycycloalkyl, —(C(H)(phenyl))-alkyl, —(CH₂)₀₋₃heteroaryl, and —(C(H)(heteroaryl))-alkyl;
is selected from —(CH₂)₀₋₃-aryl, or R₄ and R₅ together with the nitrogen atom to which they are attached can form a carbon-containing 5-, or 6-membered saturated heterocyclic ring, optionally containing an additional heteroatom selected from N, and O, which may be optionally mono- or di-substituted with substituents selected from oxo, alkyl, alkoxy, OH, halo and CF₃; and R₆ is H, small alkyl, and —(CH₂)-phenyl.
As noted above, R₁ is:
(i) Formula (II),

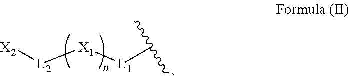

wherein:
n is 0 or 1;
L₁ is a linker selected from a single bond, —(CH₂)₁₋₃—, —(CH)(alkyl)-, and —C(alkyl)(alkyl)-;
X₁ is phenyl or a 5- or 6-membered carbon-containing aromatic ring containing one, two or three ring members that are selected from N, S, and O;
wherein X₁ may be optionally substituted with 1 or 2 substituents independently selected from alkyl, cycloalkyl, alkoxy, OH, O(cyclopropyl), CF₃, and halo;
L₂ is a linker selected from a single bond, —(CH₂)₁₋₃—, —(CH)(alkyl)-, —C(alkyl)(alkyl)-, —C(═O)—, and —SO₂—; and

X₂ is either:
(a) —NR₂OR₂₁, wherein R₂₀ is selected from H, alkyl, and —(CH₂)₀₋₃-R_{22;}
wherein R₂₁ is selected from H, alkyl, and —(CH₂)₀₋₃-R₂₃;

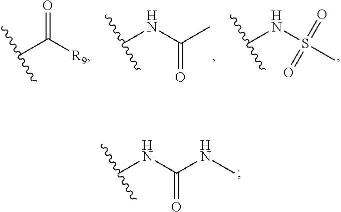

wherein R₂₂ is selected from cycloalkyl, heterocycloalkyl, aryl, heteroaryl,

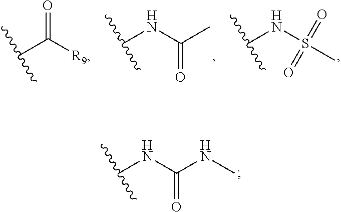

wherein R₂₃ is selected from cycloalkyl, heterocycloalkyl, aryl, heteroaryl, or
(b)₂-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O, and X₂ may be further optionally substituted with 1 or 2 substituents R₂₉ and R₃₀, wherein R₂₉ and R₃₀ are independently selected from alkyl, cycloalkyl, alkoxy, OH, oxo, CF₃, and halo; or,
(ii) Formula (III) comprising an aromatic ring A fused to a non-aromatic ring A′,

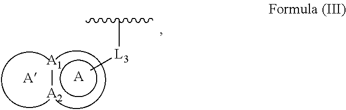

wherein:
L₃ is a linker selected from a single bond, —(CH₂)₁₋₃—, —(CH)(alkyl)-, and —C(alkyl)(alkyl)-; A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, O(cyclopropyl), CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R₇ is selected from alkyl, alkoxy, and —(CH₂)₀₋₃-R₈,

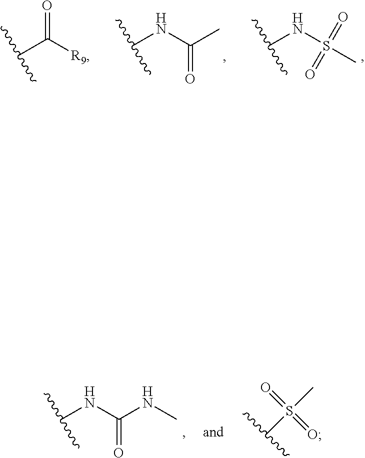

wherein R₈ is selected from cycloalkyl, heterocycloalkyl, aryl, heteroaryl, wherein R₉ is selected from H, alkyl, OH, N(R₃₁)(R₃₂), and alkoxy;
wherein R₃₁ is H or small alkyl;
wherein R₃₂ is selected from H, small alkyl, and —(CH₂)₀₋₃heteroaryl;
wherein A′ may be optionally substituted with 1, 2, or 3 further substituents R₁₀, R₁₁, and R₁₇, wherein R₁₀, R₁₁, and R₁₇ are independently selected from alkyl, cycloalkyl, alkoxy, OH, oxo, CF₃, and halo, or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O, or wherein A′ may be optionally substituted with 1 or 2 further substituents R₄₃ and R₄₄ independently selected from alkyl, cycloalkyl, alkoxy, OH, oxo, CF₃, and halo, and two different carbon ring members within A′ are connected by a bridging —CH₂-group.
R₁ can be:
(i) Formula (II),

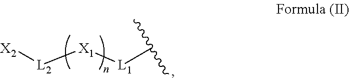

wherein:
n is 0 or 1;
L₁ is a linker selected from a single bond, —(CH₂)₁₋₃—, —(CH)(alkyl)-, and —C(alkyl)(alkyl)-;
X₁ is phenyl or a 5- or 6-membered carbon-containing aromatic ring containing one, two or three ring members that are selected from N, S, and O;
wherein X₁ may be optionally substituted with 1 or 2 substituents independently selected from alkyl, cycloalkyl, alkoxy, OH, CF₃, and halo; L₂ is a linker selected from a single bond, —(CH₂)₁₋₃—, —(CH)(alkyl)-, —C(alkyl)(alkyl)-, —C(═O)—, and —SO₂—; and

X₂ is either:
(a) —NR₂OR₂₁, wherein R₂₀ is selected from H, alkyl, and —(CH₂)₀₋₃-R_{22;}
wherein R₂₁ is selected from H, alkyl, and —(CH₂)₀₋₃-R₂₃;

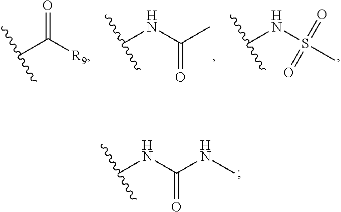

wherein R₂₂ is selected from cycloalkyl, heterocycloalkyl, aryl, heteroaryl,

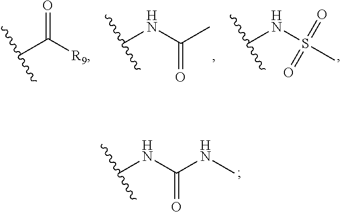

wherein R₂₃ is selected from cycloalkyl, heterocycloalkyl, aryl, heteroaryl, or
(b)₂-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O, and X₂ may be further optionally substituted with 1 or 2 substituents R₂₉ and R₃₀, wherein R₂₉ and R₃₀ are independently selected from alkyl, cycloalkyl, alkoxy, OH, oxo, CF₃, and halo; or,
(ii) Formula (III) comprising an aromatic ring A fused to a non-aromatic ring A′,

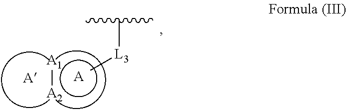

wherein:
L₃ is a linker selected from a single bond, —(CH₂)₁₋₃—, —(CH)(alkyl)-, and —C(alkyl)(alkyl)-; A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R₇ is selected from alkyl, alkoxy, and —(CH₂)₀₋₃-R₈,

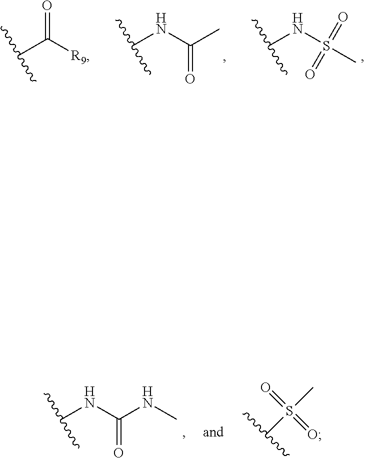

wherein R₈ is selected from cycloalkyl, heterocycloalkyl, aryl, heteroaryl, wherein R₉ is selected from H, alkyl, OH, N(R₃₁)(R₃₂), and alkoxy;
wherein R₃₁ is H or small alkyl;
wherein R₃₂ is selected from H, small alkyl, and —(CH₂)₀₋₃heteroaryl;
wherein A′ may be optionally substituted with 1, 2, or 3 further substituents R₁₀, R₁₁, and R₁₇, wherein R₁₀, R₁₁, and R₁₇ are independently selected from alkyl, cycloalkyl, alkoxy, OH, oxo, CF₃, and halo, or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O.
R₁ can be:
(i) Formula (II),

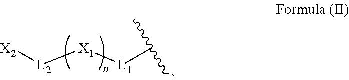

wherein:
n is 0 or 1;
L₁ is a linker selected from a single bond, —(CH₂)₁₋₃—, —(CH)(alkyl)-, and —C(alkyl)(alkyl)-;
X₁ is phenyl or a 5- or 6-membered carbon-containing aromatic ring containing one, two or three
wherein X₁ may be optionally substituted with 1 or 2 substituents independently selected from alkyl, cycloalkyl, alkoxy, OH, CF₃, and halo;
L₂ is a linker selected from a single bond, —(CH₂)₁₋₃—, —(CH)(alkyl)-, —C(alkyl)(alkyl)-, —C(═O)—, and —SO₂—; and

X₂ is either:
(a) —NR₂OR₂₁, wherein R₂₀ is selected from H, alkyl, and —(CH₂)₀₋₃-R_{22;}
wherein R₂₁ is selected from H, alkyl, and —(CH₂)₀₋₃-R₂₃;

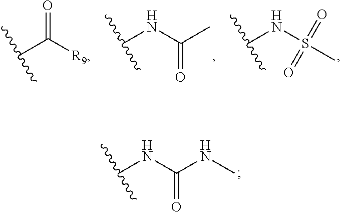

wherein R₂₂ is selected from cycloalkyl, heterocycloalkyl, aryl, heteroaryl,

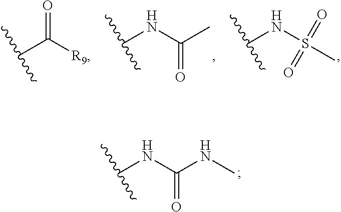

wherein R₂₃ is selected from cycloalkyl, heterocycloalkyl, aryl, heteroaryl, or
(b)₂-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected from oxo, alkyl, cycloalkyl, OH, alkoxy, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O; or,
(ii) Formula (III) comprising an aromatic ring A fused to a non-aromatic ring A′,

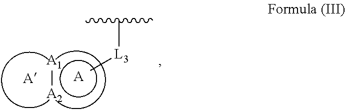

wherein:
L₃ is a linker selected from a single bond, —(CH₂)₁₋₃—, —(CH)(alkyl)-, and —C(alkyl)(alkyl)-; A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R₇ is selected from alkyl, alkoxy, and —(CH₂)₀₋₃-R₈,

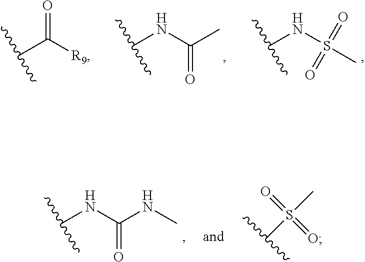

wherein R₈ is selected from cycloalkyl, heterocycloalkyl, aryl, heteroaryl, wherein R₈ is selected from H, alkyl, OH, NH₂, and alkoxy;
wherein A′ may be optionally substituted with 1, 2, or 3 further substituents R₁₀, R₁₁, and R₁₇, wherein R₁₀, R₁₁, and R₁₇ are independently selected from alkyl, cycloalkyl, alkoxy, OH, oxo, CF₃, and halo, or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O.
R₁ can be:
(i) Formula (II),

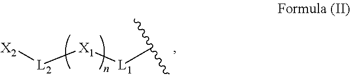

wherein:
n is 0 or 1;
L₁ is a linker selected from a single bond, —(CH₂)₁₋₃—, —(CH)(alkyl)-, and —C(alkyl)(alkyl)-; X₁ is phenyl or a 5- or 6-membered carbon-containing aromatic ring containing one, two or three
wherein X₁ may be optionally substituted with 1 or 2 substituents independently selected from alkyl, cycloalkyl, alkoxy, OH, CF₃, and halo;
L₂ is a linker selected from a single bond, —(CH₂)₁₋₃—, —(CH)(alkyl)-, —C(alkyl)(alkyl)-, —C(═O)—, and —SO₂—; and

X₂ is either:
(a) —NR₂OR₂₁, wherein R₂₀ is selected from H, alkyl, and —(CH₂)₀₋₃-R_{22;}
wherein R₂₁ is selected from H, alkyl, and —(CH₂)₀₋₃-R₂₃;

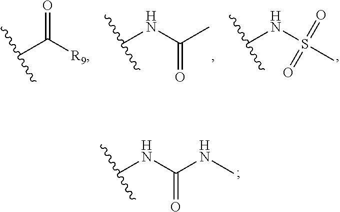

wherein R₂₂ is selected from cycloalkyl, heterocycloalkyl, aryl, heteroaryl,

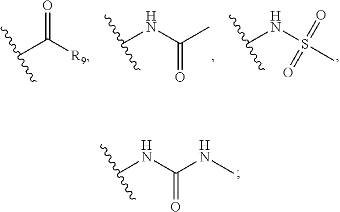

wherein R₂₃ is selected from cycloalkyl, heterocycloalkyl, aryl, heteroaryl, or
(b)₂-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected from oxo, alkyl, cycloalkyl, OH, alkoxy, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O; or,
(ii) Formula (III) comprising an aromatic ring A fused to a non-aromatic ring A′,

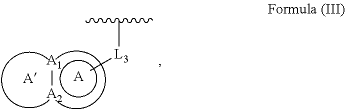

wherein:
L₃ is a linker selected from a single bond, —(CH₂)₁₋₃—, —(CH)(alkyl)-, and —C(alkyl)(alkyl)-; A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R₇ is selected from alkyl, alkoxy, and —(CH₂)₀₋₃-R₈, wherein R₈ is selected from cycloalkyl, heterocycloalkyl, aryl, heteroaryl,

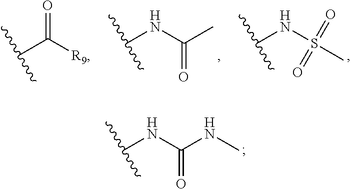

wherein R₉ is selected from H, alkyl, OH, NH₂, and alkoxy;
wherein A′ may be optionally substituted with 1 or 2 further substituents R₁₀ and R₁₁, wherein R₁₀ and Ru are independently selected from alkyl, cycloalkyl, alkoxy, OH, oxo, CF₃, and halo, or wherein A′ may be optionally substituted with 1 further substituent R₂₄ selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O.
R₁ can be Formula (II),

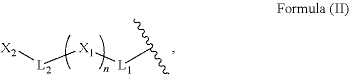

wherein:
n is 0 or 1;
L₁ is a linker selected from a single bond, —(CH₂)₁₋₃—, —(CH)(alkyl)-, and —C(alkyl)(alkyl)-; X₁ is phenyl or a 5- or 6-membered carbon-containing aromatic ring containing one, two or three ring members that are selected from N, S, and O;
wherein X₁ may be optionally substituted with 1 or 2 substituents independently selected from alkyl, cycloalkyl, alkoxy, OH, O(cyclopropyl), CF₃, and halo;
L₂ is a linker selected from a single bond, —(CH₂)₁₋₃—, —(CH)(alkyl)-, —C(alkyl)(alkyl)-, —C(═O)—, and —SO₂—; and
X₂ is either:
(a) —NR₂OR₂₁, wherein R₂₀ is selected from H, alkyl, and —(CH₂)₀₋₃-R_{22;}
wherein R₂₁ is selected from H, alkyl, and —(CH₂)₀₋₃-R₂₃;

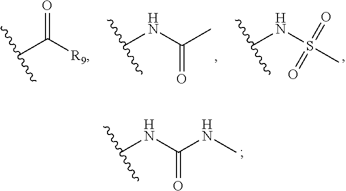

wherein R₂₂ is selected from cycloalkyl, heterocycloalkyl, aryl, heteroaryl,

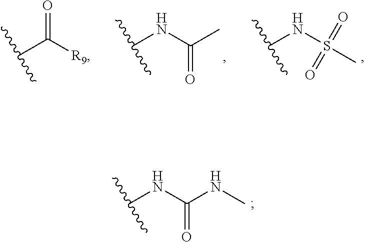

wherein R₂₃ is selected from cycloalkyl, heterocycloalkyl, aryl, heteroaryl, or
(b)₂-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O, and X₂ may be further optionally substituted with 1 or 2 substituents R₂₉ and R₃₀, wherein R₂₉ and R₃₀ are independently selected from alkyl, cycloalkyl, alkoxy, OH, oxo, CF₃, and halo.
R₁ can be Formula (II),

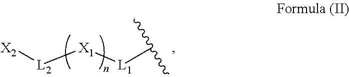

wherein:
n is 0 or 1;
L₁ is a linker selected from a single bond, —(CH₂)₁₋₃—, —(CH)(alkyl)-, and —C(alkyl)(alkyl)-; X₁ is phenyl or a 5- or 6-membered carbon-containing aromatic ring containing one, two or three ring members that are selected from N, S, and O;
wherein X₁ may be optionally substituted with 1 or 2 substituents independently selected from alkyl, cycloalkyl, alkoxy, OH, CF₃, and halo;
L₂ is a linker selected from a single bond, —(CH₂)₁₋₃—, —(CH)(alkyl)-, —C(alkyl)(alkyl)-, —C(═O)—, and —SO₂—; and

X₂ is either:
(a) —NR₂OR₂₁, wherein R₂₀ is selected from H, alkyl, and —(CH₂)₀₋₃-R_{22;}
wherein R₂₁ is selected from H, alkyl, and —(CH₂)₀₋₃-R₂₃;

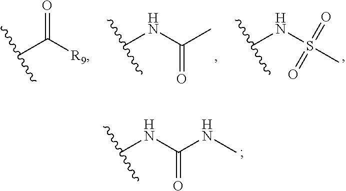

wherein R₂₂ is selected from cycloalkyl, heterocycloalkyl, aryl, heteroaryl,

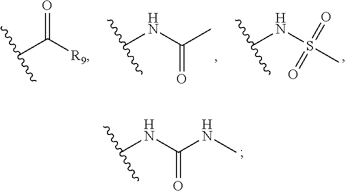

wherein R₂₃ is selected from cycloalkyl, heterocycloalkyl, aryl, heteroaryl, or
(b)₂-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected from alkyl, cycloalkyl, alkoxy, OH, Oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O, and X₂ may be further optionally substituted with 1 or 2 substituents R₂₉ and R₃₀, wherein R₂₉ and R₃₀ are independently selected from alkyl, cycloalkyl, alkoxy, OH, oxo, CF₃, and halo.
R₁ can be Formula (II),

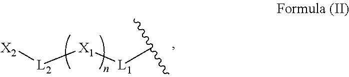

wherein:
n is 0 or 1;
L₁ is a linker selected from a single bond, —(CH₂)₁₋₃—, —(CH)(alkyl)-, and —C(alkyl)(alkyl)-;
X₁ is phenyl or a 5- or 6-membered carbon-containing aromatic ring containing one, two or three ring members that are selected from N, S, and O;
wherein X₁ may be optionally substituted with 1 or 2 substituents independently selected from alkyl, cycloalkyl, alkoxy, OH, CF₃, and halo;
L₂ is a linker selected from a single bond, —(CH₂)₁₋₃—, —(CH)(alkyl)-, —C(alkyl)(alkyl)-, —C(═O)—, and —SO₂—; and
X₂ is either:
(a) —NR₂OR₂₁, wherein R₂₀ is selected from H, alkyl, and —(CH₂)₀₋₃-R_{22;}
wherein R₂₁ is selected from H, alkyl, and —(CH₂)₀₋₃-R₂₃;

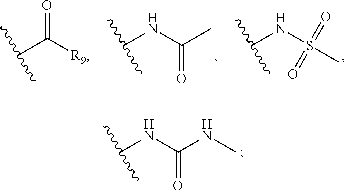

wherein R₂₂ is selected from cycloalkyl, heterocycloalkyl, aryl, heteroaryl,

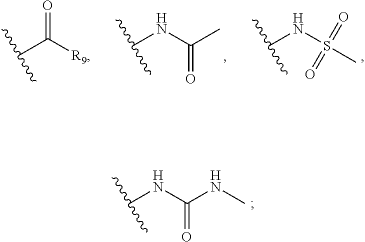

wherein R₂₃ is selected from cycloalkyl, heterocycloalkyl, aryl, heteroaryl, or
(b)₂-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O.
R₁ can be Formula (III) comprising an aromatic ring A fused to a non-aromatic ring A′,

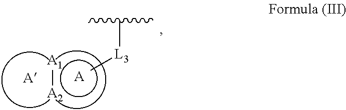

wherein:
L₃ is a linker selected from a single bond, —(CH₂)₁₋₃—, —(CH)(alkyl)-, and —C(alkyl)(alkyl)-; A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, O(cyclopropyl), CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R₇ is selected from alkyl, alkoxy, and —(CH₂) o 3-R₈,
wherein R₈ is selected from cycloalkyl, heterocycloalkyl, aryl, heteroaryl,

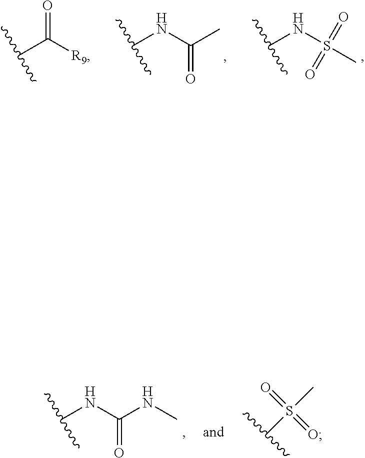

wherein R₉ is selected from H, alkyl, OH, N(R₃₁)(R₃₂), and alkoxy;
wherein R₃₁ is H or small alkyl;
wherein R₃₂ is selected from H, small alkyl, and —(CH₂)₀₋₃heteroaryl;
wherein A′ may be optionally substituted with 1, 2, or 3 further substituents R₁₀, R₁₁, and R₁₇, wherein R₁₀, R₁₁, and R₁₇ are independently selected from alkyl, cycloalkyl, alkoxy, OH, oxo, CF₃, and halo, or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O, or wherein A′ may be optionally substituted with 1 or 2 further substituents R₄₃ and R₄₄ independently selected from alkyl, cycloalkyl, alkoxy, OH, oxo, CF₃, and halo, and two different carbon ring members within A′ are connected by a bridging —CH₂-group.
R₁ can be Formula (III) comprising an aromatic ring A fused to a non-aromatic ring A′,

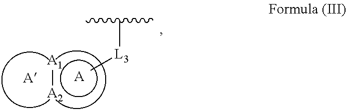

wherein:
L₃ is a linker selected from a single bond, —(CH₂)₁₋₃—, —(CH)(alkyl)-, and —C(alkyl)(alkyl)-; A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R₇ is selected from alkyl, alkoxy, and —(CH₂) o-3-R₈,

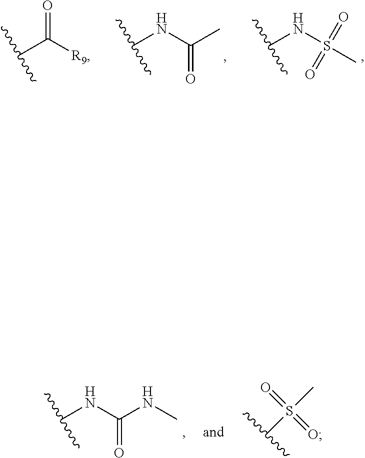

wherein R₈ is selected from cycloalkyl, heterocycloalkyl, aryl, heteroaryl, wherein R₉ is selected from H, alkyl, OH, N(R₃₁)(R₃₂), and alkoxy;
wherein R₃₁ is H or small alkyl;
wherein R₃₂ is selected from H, small alkyl, and —(CH₂)₀₋₃heteroaryl;
wherein A′ may be optionally substituted with 1, 2, or 3 further substituents R₁₀, R₁₁, and R₁₇, wherein R₁₀, R₁₁, and R₁₇ are independently selected from alkyl, cycloalkyl, alkoxy, OH, oxo, CF₃, and halo, or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O.
R₁ can be Formula (III) comprising an aromatic ring A fused to a non-aromatic ring A′,

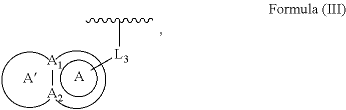

wherein:
L₃ is a linker selected from a single bond, —(CH₂)₁₋₃—, —(CH)(alkyl)-, and —C(alkyl)(alkyl)-; A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, CF₃, and halo; A′ is a non-aromatic carbon-containing monocyclic ring containing 5, 6, or 7, ring members, wherein at least one ring member is independently selected from N, S, and O;
wherein A′ may be optionally substituted with one R₇, wherein R₇ is selected from alkyl, alkoxy, and —(CH₂) o 3-R₈,

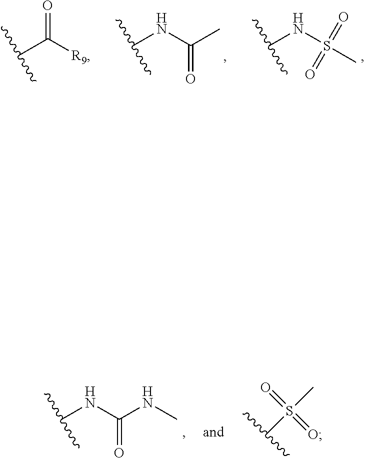

wherein R₈ is selected from cycloalkyl, heterocycloalkyl, aryl, heteroaryl, wherein R₉ is selected from H, alkyl, OH, NH₂, and alkoxy;
wherein A′ may be optionally substituted with 1, 2, or 3 further substituents R₁₀, R₁₁, and R₁₇, wherein R₁₀, R₁₁, and R₁₇ are independently selected from alkyl, cycloalkyl, alkoxy, OH, oxo, CF₃, and halo, or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O.
R₁ can be Formula (III) comprising an aromatic ring A fused to a non-aromatic ring A′,

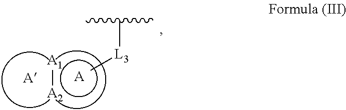

wherein:
L₃ is a linker selected from a single bond, —(CH₂)₁₋₃—, —(CH)(alkyl)-, and —C(alkyl)(alkyl)-; A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R₇ is selected from alkyl, alkoxy, and —(CH₂)₀₋₃-R₈,

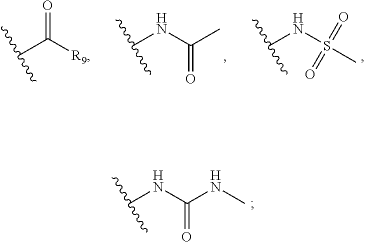

wherein R₈ is selected from cycloalkyl, heterocycloalkyl, aryl, heteroaryl, wherein R₉ is selected from H, alkyl, OH, NH₂, and alkoxy;
wherein A′ may be optionally substituted with 1 or 2 further substituents R₁₀ and R₁, wherein R₁₀ and R₁₁ are independently selected from alkyl, cycloalkyl, alkoxy, OH, oxo, CF₃, and halo, or wherein A′ may be optionally substituted with 1 further substituent R₂₄ selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O.

Preferably, n is 1.
L₁ can be a linker selected from a single bond and —(CH₂)₁₋₃—.
L₁ can be a single bond.
L₂ can be a linker selected from a single bond, —(CH₂)₁₋₃—, —(CH)(alkyl)-, —C(alkyl)(alkyl)-, and —SO₂—.
L₂ is a linker selected from a single bond, —(CH₂)₁₋₃—, and —C(═O)—.
L₂ can be a linker selected from a single bond and —(CH₂)₁₋₃—.
L₁ can be a single bond.
L₁ and L₂ can each independently be a linker selected from a single bond and —(CH₂)₁₋₃—. L₁ and L₂ can both be a single bond.
X₁ can be selected from thiophene, phenyl and pyridyl.
X₁ can be selected from phenyl and pyridyl.
X₁ can be phenyl.
X₁ can be optionally substituted with 1 or 2 substituents independently selected from small alkyl, cyclopropyl, small alkoxy, OH, CF₃, and halo.
X₁ can be selected from thiophene, phenyl and pyridyl, which may be optionally substituted with 1 or 2 substituents independently selected from small alkyl, cyclopropyl, small alkoxy, OH, CF₃, and halo.
X₁ can be selected from phenyl and pyridyl, which may be optionally substituted with 1 or 2 substituents independently selected from small alkyl, cyclopropyl, small alkoxy, OH, CF₃, and halo.

When X₂ is-NR₂OR₂₁, L₂ can be a linker selected from a single bond, —(CH₂)₁₋₃—, —(CH)(alkyl)-, —C(alkyl)(alkyl)-, and —SO₂—.

When X₂ is-NR₂OR₂₁, L₂ can be a linker selected from a single bond or—CH₂-.

When X₂ is-NR₂OR₂₁, X₁ can be phenyl or pyridyl.

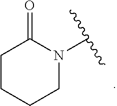 [SMILES: *N1CCCCC1=O]

In some embodiments, X₂ is not

In some embodiments, X₂ is not a cyclic amide.

X₂ can be 2-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O, and X₂ may be further optionally substituted with 1 or 2 substituents R₂₉ and R₃₀, wherein R₂₉ and R₃₀ are independently selected from alkyl, cycloalkyl, alkoxy,

OH, oxo, CF₃, and halo.

X₂ can be 2-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O.

X₂ can be a non-aromatic carbon-containing mono- or bi-cyclic ring containing 5, 6, or 7 ring members, wherein at least one ring member is N.

X₂ can be a non-aromatic carbon-containing mono- or bi-cyclic ring containing 5, 6, or 7 ring members, wherein at least one ring member is N, and wherein, when X₂ is a bi-cyclic ring system, the bicyclic ring system is fused.

X₂ can be a non-aromatic carbon-containing mono- or bi-cyclic ring containing 5 or 6 ring members, wherein at least one ring member is N.

X₂ can be a non-aromatic carbon-containing mono- or bi-cyclic ring containing 5 or 6 ring members, wherein at least one ring member is N, and wherein, when X₂ is a bi-cyclic ring system, the bicyclic ring system is fused.

X₂ can be a non-aromatic carbon-containing mono- or bi-cyclic ring containing 5, 6, or 7 ring members, wherein one ring member is N.

X₂ can be a non-aromatic carbon-containing mono- or bi-cyclic ring containing 5, 6, or 7 ring members, wherein one ring member is N, and wherein, when X₂ is a bi-cyclic ring system, the bicyclic ring system is fused.

X₂ can be a non-aromatic carbon-containing mono- or bi-cyclic ring containing 5 or 6 ring members, wherein one ring member is N.

X₂ can be a non-aromatic carbon-containing mono- or bi-cyclic ring containing 5 or 6 ring members, wherein one ring member is N, and wherein, when X₂ is a bi-cyclic ring system, the bicyclic ring system is fused.

X₂ may be attached to the remainder of the molecule via a N ring member of X_{2.}

X₂ can be a non-aromatic carbon-containing mono- or bi-cyclic ring containing 5 or 6 ring members, wherein at least one ring member is N, and wherein X₂ is attached to the remainder of the molecule via a N ring member of X₂.

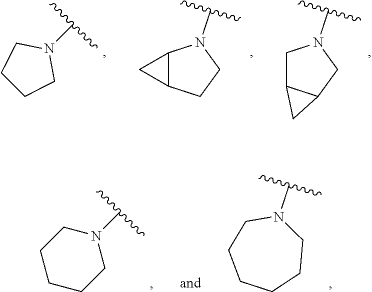 [SMILES: *N1CCCC1 ; *N1CCC2CC21 ; *N1CC2CC2C1 ; *N1CCCCC1 ; *N1CCCCCC1]

X₂ can be selected from the structures wherein X₂ may be optionally substituted with 1 or 2 substituents R₁₈ and R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O, and X₂ may be further optionally substituted with 1 or 2 substituents R₂₉ and R₃₀, wherein R₂₉ and R₃₀ are independently selected from alkyl, cycloalkyl, alkoxy, OH, oxo, CF₃, and halo.

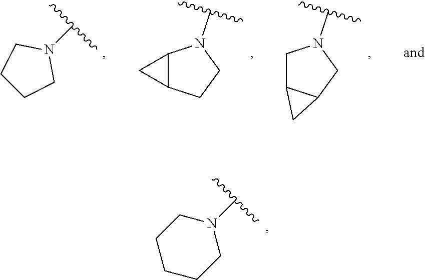 [SMILES: *N1CCCC1 ; *N1CCC2CC21 ; *N1CC2CC2C1 ; *N1CCCCC1]

X₂ can be selected from the structures wherein X₂ may be optionally substituted with 1 or 2 substituents R₁₈ and R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O, and X₂ may be further optionally substituted with 1 or 2 substituents R₂₉ and R₃₀, wherein R₂₉ and R₃₀ are independently selected from alkyl, cycloalkyl, alkoxy, OH, oxo, CF₃, and halo.

X₂ can be a non-aromatic carbon-containing mono- or bi-cyclic ring containing 5 or 6 ring members, wherein at least one ring member is N; when X₂ is a bi-cyclic ring system, the bicyclic ring system is fused; and X₂ is attached to the remainder of the molecule via a N ring member of X₂.

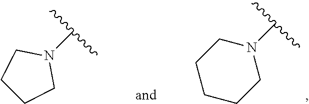 [SMILES: *N1CCCC1 ; *N1CCCCC1]

X₂ can be selected from the structures wherein X₂ may optionally be substituted with 1 or 2 substituents independently selected from alkyl, cyclopropyl, (C₁-C₆)alkoxy, OH, CN, CF₃, oxo, and halo.

X₂ can be a non-aromatic carbon-containing mono- or bi-cyclic ring containing 5 or 6 ring members, wherein at least one ring member is N; when X₂ is a bi-cyclic ring system, the bicyclic ring system is fused; and X₂ is attached to the remainder of the molecule via a N ring member of X₂.

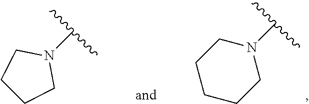 [SMILES: *N1CCCC1 ; *N1CCCCC1]

X₂ can be selected from the structures wherein X₂ may optionally be substituted with 1 or 2 substituents independently selected from alkyl, cyclopropyl, (C₁-C₆)alkoxy, OH, CN, CF₃, and halo.

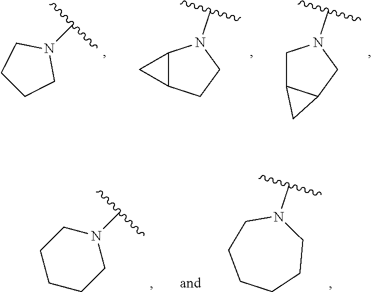 [SMILES: *N1CCCC1 ; *N1CCC2CC21 ; *N1CC2CC2C1 ; *N1CCCCC1 ; *N1CCCCCC1]

X₂ can be selected from the structures wherein X₂ is substituted with an oxo substituent, and X₂ may be optionally substituted with 1 or 2 substituents R₁₈ and R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O, and X₂ may be further optionally substituted with R₃₀, wherein R₃₀ is selected from alkyl, cycloalkyl, alkoxy, OH, oxo, CF₃, and halo.

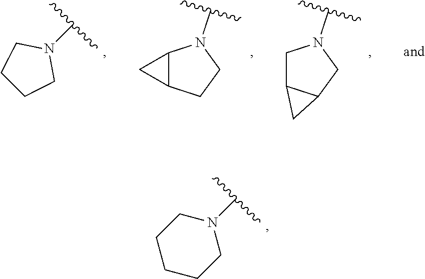 [SMILES: *N1CCCC1 ; *N1CCC2CC21 ; *N1CC2CC2C1 ; *N1CCCCC1]

X₂ can be selected from the structures wherein X₂ is substituted with an oxo substituent, and X₂ may be optionally substituted with 1 or 2 substituents R₁₈ and R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O, and X₂ may be further optionally substituted with R₃₀, wherein R₃₀ is selected from alkyl, cycloalkyl, alkoxy, OH, oxo, CF₃, and halo.

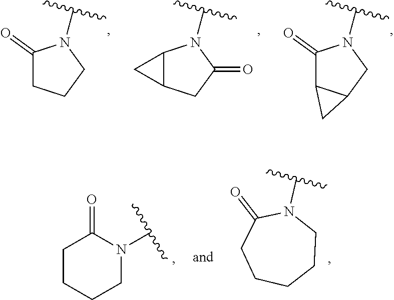 [SMILES: *N1CCCC1=O ; *N1C(=O)CC2CC21 ; *N1CC2CC2C1=O ; *N1CCCCC1=O ; *N1CCCCCC1=O]

X₂ can be selected from the structures wherein X₂ may be optionally substituted with 1 or 2 substituents R₁₈ and R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O, and X₂ may be further optionally substituted with R₃₀, wherein R₃₀ is selected from alkyl, cycloalkyl, alkoxy, OH, oxo, CF₃, and halo.

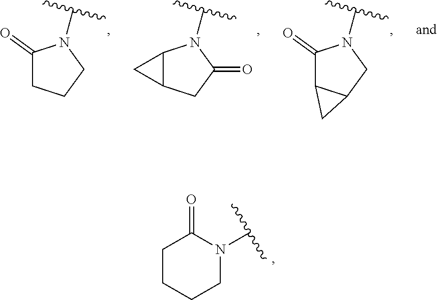 [SMILES: *N1CCCC1=O ; *N1C(=O)CC2CC21 ; *N1CC2CC2C1=O ; *N1CCCCC1=O]

X₂ can be selected from the structures wherein X₂ may be optionally substituted with 1 or 2 substituents R₁₈ and R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O, and X₂ may be further optionally substituted with 1 or 2 substituents R₂₉ and R₃₀, wherein R₂₉ and R₃₀ are independently selected from alkyl, cycloalkyl, alkoxy, OH, oxo, CF₃, and halo.

X₂ can be a cyclic amide.
R₁₈ and R₁₉ can be independently selected from alkyl, cycloalkyl, alkoxy, OH, CF₃, and halo.
R₂₀ can be selected from H and small alkyl.
R₂₁ can be selected from alkyl and —(CH₂)₀₋₃-R_{23.}

Preferably, Formula (II) is represented by Formula (IIb),

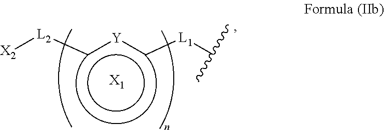

wherein:
n is 0 or 1;
L₁ is a linker selected from a single bond, —(CH₂)₁₋₃—, —(CH)(alkyl)-, and —C(alkyl)(alkyl)-;
Y is selected from C, N, O and S;
X₁ is phenyl or 5- or 6-membered aromatic ring containing one, two or three ring members that are selected from N, S, and O; wherein X₁ may be optionally substituted with 1 or 2 substituents independently selected from alkyl, cycloalkyl, alkoxy, OH, O(cyclopropyl), CF₃, and halo; L₂ is a linker selected from a single bond, —(CH₂)₁₋₃—, —(CH)(alkyl)-, —C(alkyl)(alkyl)-, —C(═O)—, and —SO₂—; and

X₂ is either:
(a) —NR₂OR₂₁, wherein R₂₀ is selected from H, alkyl, and —(CH₂)₀₋₃-R_{22;}
wherein R₂₁ is selected from H, alkyl, and —(CH₂)₀₋₃-R₂₃;

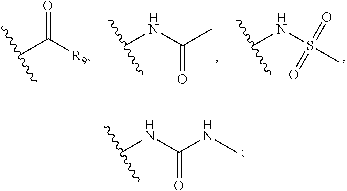

wherein R₂₂ is selected from cycloalkyl, heterocycloalkyl, aryl, heteroaryl,

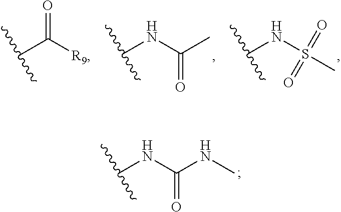

wherein R₂₃ is selected from cycloalkyl, heterocycloalkyl, aryl, heteroaryl, wherein R₉ is selected from H, alkyl, OH, NH₂, and alkoxy; or
(b)₂-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O, and X₂ may be further optionally substituted with 1 or 2 substituents R₂₉ and R₃₀, wherein R₂₉ and R₃₀ are independently selected from alkyl, cycloalkyl, alkoxy, OH, oxo, CF₃, and halo.

Formula (II) can be represented by Formula (IIb),

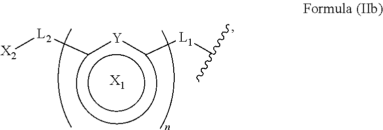

wherein:
n is 0 or 1;
L₁ is a linker selected from a single bond, —(CH₂)₁₋₃—, —(CH)(alkyl)-, and —C(alkyl)(alkyl)-; Y is selected from C, N, O and S; X₁ is phenyl or 5- or 6-membered aromatic ring containing one, two or three ring members that are selected from N, S, and O; wherein X₁ may be optionally substituted with 1 or 2 substituents independently selected from alkyl, cycloalkyl, alkoxy, OH, CF₃, and halo;
L₂ is a linker selected from a single bond, —(CH₂)₁₋₃—, —(CH)(alkyl)-, —C(alkyl)(alkyl)-, —C(═O)—, and —SO₂—; and

X₂ is either:
(a) —NR₂OR₂₁, wherein R₂₀ is selected from H, alkyl, and —(CH₂)₀₋₃-R_{22;}
wherein R₂₁ is selected from H, alkyl, and —(CH₂)₀₋₃-R₂₃;

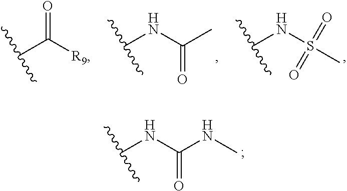

wherein R₂₂ is selected from cycloalkyl, heterocycloalkyl, aryl, heteroaryl,

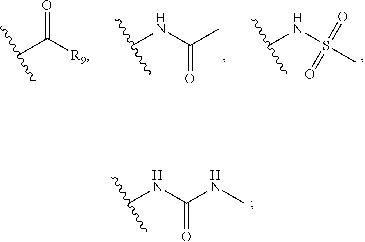

wherein R₂₃ is selected from cycloalkyl, heterocycloalkyl, aryl, heteroaryl, wherein R₉ is selected from H, alkyl, OH, NH₂, and alkoxy; or
(b)₂-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O, and X₂ may be further optionally substituted with 1 or 2 substituents R₂₉ and R₃₀, wherein R₂₉ and R₃₀ are independently selected from alkyl, cycloalkyl, alkoxy, OH, oxo, CF₃, and halo.

Formula (II) can be represented by Formula (IIb),

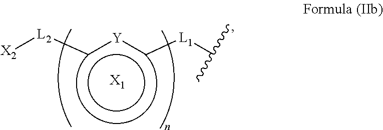

wherein:
n is 0 or 1;
L₁ is a linker selected from a single bond, —(CH₂)₁₋₃—, —(CH)(alkyl)-, and —C(alkyl)(alkyl)-; Y is selected from C, N, O and S; X₁ is phenyl or 5- or 6-membered aromatic ring containing one, two or three ring members that are selected from N, S, and O; wherein X₁ may be optionally substituted with 1 or 2 substituents independently selected from alkyl, cycloalkyl, alkoxy, OH, CF₃, and halo;
L₂ is a linker selected from a single bond, —(CH₂)₁₋₃—, —(CH)(alkyl)-, —C(alkyl)(alkyl)-, —C(═O)—, and —SO₂—; and

X₂ is either:
(a) —NR₂OR₂₁, wherein R₂₀ is selected from H, alkyl, and —(CH₂)₀₋₃-R_{22;}
wherein R₂₁ is selected from H, alkyl, and —(CH₂)₀₋₃-R₂₃;

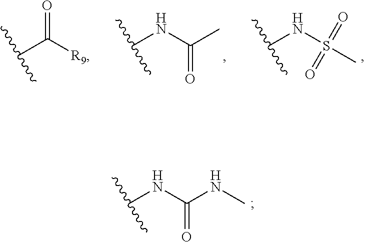

wherein R₂₂ is selected from cycloalkyl, heterocycloalkyl, aryl, heteroaryl,

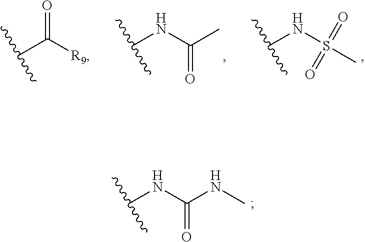

wherein R₂₃ is selected from cycloalkyl, heterocycloalkyl, aryl, heteroaryl, wherein R₉ is selected from H, alkyl, OH, NH₂, and alkoxy; or
(b)₂-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O.
L₃ can be a linker selected from a single bond and —(CH₂)₁₋₃—.
L₃ can be a single bond.

In some embodiments, A does not contain both N and S ring members.

A can be phenyl or a 5- or 6-membered carbon-containing aromatic ring containing one, two or three ring members that are selected from N and O.

A can be phenyl or a 6-membered carbon-containing aromatic ring containing one, two or three nitrogen ring members.

A can be phenyl, pyridyl, or pyrimidyl. A can be phenyl or pyridyl. A can be phenyl. A can be pyridyl.

A can be optionally substituted with a single substituent, wherein the substituent is a halo substituent.

A can be optionally substituted with a single substituent, wherein the substituent is F.

A can be substituted with a single substituent, wherein the substituent is a halo substituent.

A can be substituted with a single substituent, wherein the substituent is F.

In some embodiments, A is not substituted with any optional substituents.

A can be phenyl, pyridyl, or pyrimidyl, wherein A is not further substituted with any optional substituents. A can be phenyl or pyridyl, wherein A is not further substituted with any optional substituents.

A can be phenyl or pyridyl, wherein A is optionally substituted with a single substituent, wherein the substituent is halo.

A can be phenyl or pyridyl, wherein A is substituted with a single substituent, wherein the substituent is halo.

A can be phenyl or pyridyl, wherein A is optionally substituted with a single substituent, wherein the substituent is F.

A can be phenyl or pyridyl, wherein A is substituted with a single substituent, wherein the substituent is

F.

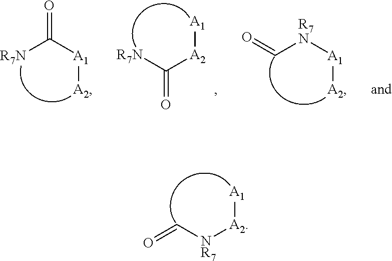

When A′ is a cyclic amide, A′ can be selected from

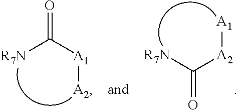

When A′ is a cyclic amide, A′ can be selected from

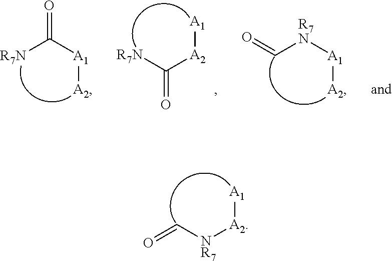

A′ can be a cyclic amide, and A′ can be selected from

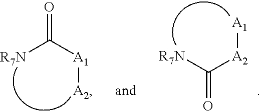

A′ is a cyclic amide, and A′ can be selected from A′ can be a non-aromatic carbon-containing monocyclic ring containing 5, 6, or 7 ring members, wherein at least one ring member is N.

A′ may be a non-aromatic carbon-containing monocyclic ring containing 6 or 7 ring members, wherein at least one ring member is N.

A′ can be a non-aromatic carbon-containing monocyclic ring containing 7 ring members, wherein at least one ring member is N.

A′ can be a non-aromatic carbon-containing monocyclic ring containing 6 ring members, wherein at least one ring member is N.

A′ can be a non-aromatic carbon-containing monocyclic ring containing 5, 6, or 7 ring members, wherein one ring member is N.

A′ can be a non-aromatic carbon-containing monocyclic ring containing 7 ring members, wherein one ring member is N or wherein one ring member is N and a second ring member is O.

A′ can be a non-aromatic carbon-containing monocyclic ring containing 7 ring members, wherein one ring member is N.

A′ can be a non-aromatic carbon-containing monocyclic ring containing 7 ring members, wherein one ring member is N and a second ring member is O.

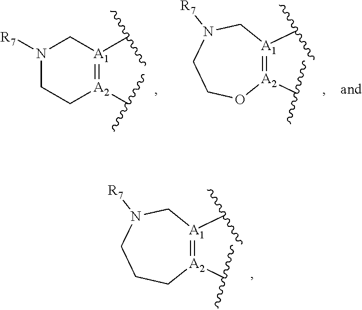

A′ can be selected from and wherein A′ may be optionally substituted with 1, 2, or 3 further substituents R₁₀, R₁₁, and R₁₇, wherein R₁₀, R₁₁, and R₁₇, are independently selected from alkyl, cycloalkyl, alkoxy, OH, oxo, CF₃, and halo, or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O, or wherein A′ may be optionally substituted with 1 or 2 further substituents R₄₃ and R₄₄ independently selected from alkyl, cycloalkyl, alkoxy, OH, oxo, CF₃, and halo, and two different carbon ring members within A′ may be connected by a bridging —CH₂-group.

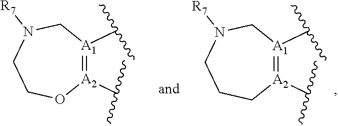

A′ can be selected from and wherein A′ may be optionally substituted with 1, 2, or 3 further substituents R₁₀, R₁₁, and R₁₇, wherein R₁₀, R₁₁, and R₁₇, are independently selected from alkyl, cycloalkyl, alkoxy, OH, oxo, CF₃, and halo, or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O, or wherein A′ may be optionally substituted with 1 or 2 further substituents R₄₃ and R₄₄ independently selected from alkyl, cycloalkyl, alkoxy, OH, oxo, CF₃, and halo, and two different carbon ring members within A′ may be connected by a bridging —CH₂-group.

A′ can be a non-aromatic carbon-containing monocyclic ring containing 6 ring members, wherein one ring member is N.

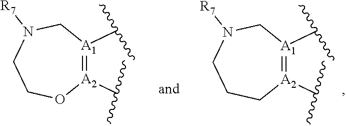

A′ can be selected from wherein A′ may be optionally substituted with 1, 2, or 3 further substituents R₁₀ R₁₁, and R₁₇, wherein R₁₀, R₁₁, and R₁₇, are independently selected from alkyl, cycloalkyl, alkoxy, OH, oxo, CF₃, and halo, or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O.

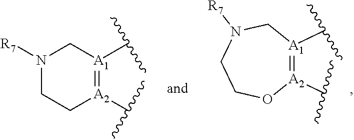

A′ can be selected from and wherein A′ may be optionally substituted with 1, 2, or 3 further substituents R₁₀, R₁₁, and R₁₇, wherein R₁₀, R₁₁, and R₁₇, are independently selected from alkyl, cycloalkyl, alkoxy, OH, oxo, CF₃, and halo, or wherein A′ may be optionally substituted with 1 or 2 further substituents R₂₄ and R₂₇ independently selected from alkyl, cycloalkyl, alkoxy, OH, oxo,

CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O, or wherein A′ may be optionally substituted with 1 or 2 further substituents R₄₃ and R₄₄ independently selected from alkyl, cycloalkyl, alkoxy, OH, oxo, CF₃, and halo, and two different carbon ring members within A′ may be connected by a bridging —CH₂-group.

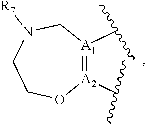

A′ can be and wherein A′ may be optionally substituted with 1, 2, or 3 further substituents R₁₀, R₁₁, and R₁₇, wherein R₁₀, R₁₁, and R₁₇, are independently selected from alkyl, cycloalkyl, alkoxy, OH, oxo, CF₃, and halo, or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O, or wherein A′ may be optionally substituted with 1 or 2 further substituents R₄₃ and R₄₄ independently selected from alkyl, cycloalkyl, alkoxy, OH, oxo, CF₃, and halo, and two different carbon ring members within A′ may be connected by a bridging —CH₂-group.

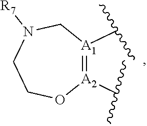

A′ can be wherein A′ may be optionally substituted with 1, 2, or 3 further substituents R₁₀, R₁₁, and R₁₇, wherein R₁₀, R₁₁, and R₁₇, are independently selected from alkyl, cycloalkyl, alkoxy, OH, oxo, CF₃, and halo, or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O.

A′ can be further substituted with an oxo substituent.

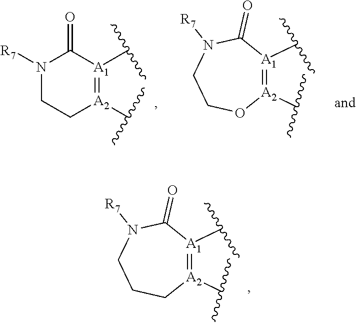

A′ can be selected from and wherein A′ may be optionally substituted with 1 or 2 further substituents R₁₀ and R₁₁, wherein R₁₀ and R₁₁ are independently selected from alkyl, cycloalkyl, alkoxy, OH, oxo, CF₃, and halo, or wherein A′ may be optionally substituted with 1 further substituent R₂₄ selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O, or wherein A′ may be optionally substituted with 1 further substituent R₄₃ selected from alkyl, cycloalkyl, alkoxy, OH, oxo, CF₃, and halo, and two different carbon ring members within A′ may be connected by a bridging —CH₂-group.

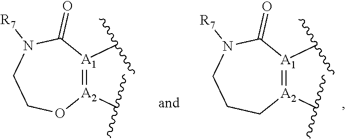

A′ can be selected from and wherein A′ may be optionally substituted with 1 or 2 further substituents R₁₀ and R₁₁, wherein R₁₀ and R₁₁ are independently selected from alkyl, cycloalkyl, alkoxy, OH, oxo, CF₃, and halo, or wherein A′ may be optionally substituted with 1 further substituent R₂₄ selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O, or wherein A′ may be optionally substituted with 1 further substituent R₄₃ selected from alkyl, cycloalkyl, alkoxy, OH, oxo, CF₃, and halo, and two different carbon ring members within A′ may be connected by a bridging —CH₂-group.

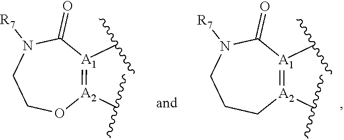

A′ can be selected from wherein A′ may be optionally substituted with 1 or 2 further substituents R₁₀ and R₁₁, wherein R₁₀ and R₁₁ are independently selected from alkyl, cycloalkyl, alkoxy, OH, oxo, CF₃, and halo, or wherein A′ may be optionally substituted with 1 further substituent R₂₄ selected from alkyl, cycloalkyl, alkoxy, OH, oxo, CF₃, and halo and 2 further substituents
R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O.

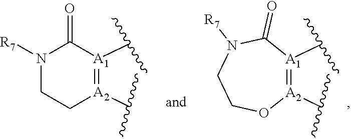

A′ can be selected from and wherein A′ may be optionally substituted with 1 or 2 further substituents R₁₀ and R₁₁, wherein R₁₀ and R₁₁ are independently selected from alkyl, cycloalkyl, alkoxy, OH, oxo, CF₃, and halo, or wherein A′ may be optionally substituted with 1 further substituent R₂₄ selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O, or wherein A′ may be optionally substituted with 1 further substituent R₄₃ selected from alkyl, cycloalkyl, alkoxy, OH, oxo, CF₃, and halo, and two different carbon ring members within A′ may be connected by a bridging —CH₂-group.

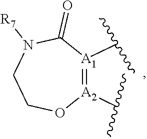

A′ can be and wherein A′ may be optionally substituted with 1 or 2 further substituents R₁₀ and R₁₁, wherein R₁₀ and R₁₁ are independently selected from alkyl, cycloalkyl, alkoxy, OH, oxo, CF₃, and halo, or wherein A′ may be optionally substituted with 1 further substituent R₂₄ selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O, or wherein A′ may be optionally substituted with 1 further substituent R₄₃ selected from alkyl, cycloalkyl, alkoxy, OH, oxo, CF₃, and halo, and two different carbon ring members within A′ may be connected by a bridging —CH₂-group.

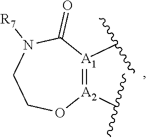

A′ can be wherein A′ may be optionally substituted with 1 or 2 further substituents R₁₀ and R₁₁, wherein R₁₀ and R₁₁ are independently selected from alkyl, cycloalkyl, alkoxy, OH, oxo, CF₃, and halo, or wherein A′ may be optionally substituted with 1 further substituent R₂₄ selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O.

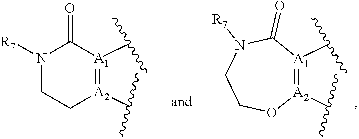

A′ can be selected from and wherein A′ has no further substituents.

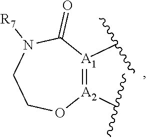

A′ can be and has no further substituents.

In some embodiments R₇ is not optional. R₇ may be present.

A′ can be a non-aromatic carbon-containing monocyclic ring containing 5, 6, or 7 ring members, wherein at least one ring member is N, wherein R₇, when present, is bonded to A′ via a N ring member.

A′ can be a non-aromatic carbon-containing monocyclic ring containing 6 or 7 ring members, wherein at least one ring member is N, wherein R₇, when present, is bonded to A′ via a N ring member.

A′ can be a non-aromatic carbon-containing monocyclic ring containing 6 ring members, wherein at least one ring member is N, wherein R₇, when present, is bonded to A′ via a N ring member.

A′ can be a non-aromatic carbon-containing monocyclic ring containing 5, 6, or 7 ring members, wherein one ring member is N, wherein R₇, when present, is bonded to A′ via a N ring member.

A′ can be a non-aromatic carbon-containing monocyclic ring containing 6 or 7 ring members, wherein one ring member is N, wherein R₇, when present, is bonded to A′ via a N ring member.

A′ can be a non-aromatic carbon-containing monocyclic ring containing 6 ring members, wherein one ring member is N, wherein R₇, when present, is bonded to A′ via a N ring member.

A′ can be a non-aromatic carbon-containing monocyclic ring containing 5, 6, or 7 ring members, wherein at least one ring member is N, wherein R₇ is present and bonded to A′ via a N ring member.

A′ can be a non-aromatic carbon-containing monocyclic ring containing 6 or 7 ring members, wherein at least one ring member is N, wherein R₇ is present and bonded to A′ via a N ring member.

A′ can be a non-aromatic carbon-containing monocyclic ring containing 6 ring members, wherein at least one ring member is N, wherein R₇ is present and bonded to A′ via a N ring member.

A′ can be a non-aromatic carbon-containing monocyclic ring containing 6 or 7 ring members, wherein one ring member is N, wherein R₇ is present and bonded to A′ via a N ring member.

A′ can be a non-aromatic carbon-containing monocyclic ring containing 6 ring members, wherein one ring member is N, wherein R₇ is present and bonded to A′ via a N ring member.

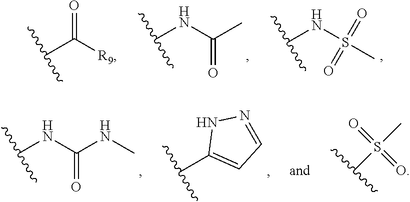

R₉ can be selected from cycloalkyl, heterocycloalkyl,
R₇ may be alkyl. R₇ may be (C₁-C₃) alkyl. R₇ may be (C₁-C₂) alkyl. R₇ may be (C₂) alkyl. R₇ may be (C₂) alkyl, wherein in the (C₂) alkyl is unsubstituted or is substituted with OH or halo substituents. R₇ may be selected from —CH₃,—CH₂—CH₃,—CH₂—CH₂—OH, CH₂—CHF₂, CH₂—CF₃, CH₂—CH₂—CH₂—OH, —CH (CH₃)₂, and —CH₂—(C═O)—NH₂. R₇ may be selected from —CH₃,—CH₂—CH₃, CH₂—CH₂—OH, CH₂—CHF₂, CH₂—CF₃, CH₂—CH₂—CH₂—OH, and —CH (CH₃)₂. R₇ may be selected from —CH₃,—CH₂—CH₃ and CH₂—CH₂—OH. R₇ may be selected from —CH₂—CH₃ and CH₂—CH₂—OH, CH₂—CHF₂, CH₂—CF₃, CH₂—CH₂—CH₂—OH, and —CH (CH₃)₂. R₇ may be selected from —CH₂—CH₃ and CH₂—CH₂—OH. R₇ can be selected from —CH₃ and CH₂—CH₂—OH. R₇ may be CH₂—CH₂—OH. R₇ can be —CH_{3.}

A′ can be a non-aromatic carbon-containing monocyclic ring containing 5, 6, or 7 ring members, wherein at least one ring member is N, wherein R₇ is (C₁-C₃) alkyl and bonded to A′ via a N ring member.

A′ can be a non-aromatic carbon-containing monocyclic ring containing 5, 6, or 7 ring members, wherein at least one ring member is N, wherein R₇ is (C₁-C₂) alkyl and bonded to A′ via a N ring member.

A′ can be a non-aromatic carbon-containing monocyclic ring containing 6 or 7 ring members, wherein at least one ring member is N, wherein R₇ is (C₁-C₃) alkyl and bonded to A′ via a N ring member.

A′ can be a non-aromatic carbon-containing monocyclic ring containing 6 or 7 ring members, wherein at least one ring member is N, wherein R₇ is (C₁-C₂) alkyl and bonded to A′ via a N ring member.

A′ can be a non-aromatic carbon-containing monocyclic ring containing 6 ring members, wherein at least one ring member is N, wherein R₇ is (C₁-C₂) alkyl and bonded to A′ via a N ring member.

A′ can be a non-aromatic carbon-containing monocyclic ring containing 5, 6, or 7 ring members, wherein one ring member is N, wherein R₇ is (C₁-C₃) alkyl and bonded to A′ via a N ring member.

A′ can be a non-aromatic carbon-containing monocyclic ring containing 5, 6, or 7 ring members, wherein one ring member is N, wherein R₇ is (C₁-C₂) alkyl and bonded to A′ via a N ring member.

A′ can be a non-aromatic carbon-containing monocyclic ring containing 6 or 7 ring members, wherein one ring member is N, wherein R₇ is (C₁-C₃) alkyl and bonded to A′ via a N ring member.

A′ can be a non-aromatic carbon-containing monocyclic ring containing 6 or 7 ring members, wherein one ring member is N, wherein R₇ is (C₁-C₂) alkyl and bonded to A′ via a N ring member.

A′ can be a non-aromatic carbon-containing monocyclic ring containing 6 ring members, wherein one ring member is N, wherein R₇ is (C₁-C₂) alkyl and bonded to A′ via a N ring member.

A′ can be a non-aromatic carbon-containing monocyclic ring containing 5, 6, or 7 ring members, wherein at least one ring member is N, wherein R₇ is selected from —CH₃,—CH₂—CH₃, CH₂—CH₂—OH, CH₂—CHF₂, CH₂—CF₃, CH₂—CH₂—CH₂—OH, —CH (CH₃)₂, and —CH₂—(C═O)-NH₂, and wherein R₇ is bonded to A′ via a N ring member.

A′ can be a non-aromatic carbon-containing monocyclic ring containing 5, 6, or 7 ring members, wherein at least one ring member is N, wherein R₇ is selected from —CH₃,—CH₂—CH₃, CH₂—CH₂—OH, CH₂—CHF₂, CH₂—CF₃, CH₂—CH₂—CH₂—OH, and —CH (CH₃)₂, and wherein R₇ is bonded to A′ via a N ring member.

A′ can be a non-aromatic carbon-containing monocyclic ring containing 5, 6, or 7 ring members, wherein at least one ring member is N, wherein R₇ is selected from —CH₃,—CH₂—CH₃ and CH₂—CH₂—OH, and wherein R₇ is bonded to A′ via a N ring member.

A′ can be a non-aromatic carbon-containing monocyclic ring containing 6 or 7 ring members, wherein at least one ring member is N, wherein R₇ is selected from —CH₃,—CH₂—CH₃, CH₂—CH₂—OH, CH₂—CHF₂, CH₂—CF₃, CH₂—CH₂—CH₂—OH, —CH (CH₃)₂, and —CH₂—(C═O)-NH₂, and wherein R₇ is bonded to A′ via a N ring member.

A′ can be a non-aromatic carbon-containing monocyclic ring containing 6 or 7 ring members, wherein at least one ring member is N, wherein R₇ is selected from —CH₃,—CH₂—CH₃, CH₂—CH₂—OH, CH₂—CHF₂, CH₂—CF₃, CH₂—CH₂—CH₂—OH, and —CH (CH₃)₂, and wherein R₇ is bonded to A′ via a N ring member.

A′ can be a non-aromatic carbon-containing monocyclic ring containing 6 or 7 ring members, wherein at least one ring member is N, wherein R₇ is selected from —CH₃,—CH₂—CH₃ and CH₂—CH₂—OH, and wherein R₇ is bonded to A′ via a N ring member.

A′ can be a non-aromatic carbon-containing monocyclic ring containing 6 ring members, wherein at least one ring member is N, wherein R₇ is selected from —CH₃,—CH₂—CH₃ and CH₂—CH₂—OH, and wherein R₇ is bonded to A′ via a N ring member.

A′ can be a non-aromatic carbon-containing monocyclic ring containing 5, 6, or 7 ring members, wherein one ring member is N, wherein R₇ is selected from —CH₃,—CH₂—CH₃, CH₂—CH₂—OH, CH₂—CHF₂, CH₂—CF₃, CH₂-CH₂—CH₂—OH, —CH (CH₃)₂, and —CH₂—(C═O)-NH₂, and wherein R₇ is bonded to A′ via a N ring member.

A′ can be a non-aromatic carbon-containing monocyclic ring containing 5, 6, or 7 ring members, wherein one ring member is N, wherein R₇ is selected from —CH₃,—CH₂—CH₃, CH₂—CH₂—OH, CH₂—CHF₂, CH₂—CF₃, CH₂-CH₂—CH₂—OH, and —CH (CH₃)₂, and wherein R₇ is bonded to A′ via a N ring member.

A′ can be a non-aromatic carbon-containing monocyclic ring containing 5, 6, or 7 ring members, wherein one ring member is N, wherein R₇ is selected from —CH₃,—CH₂—CH₃ and CH₂—CH₂—OH, and wherein R₇ is bonded to A′ via a N ring member.

A′ can be a non-aromatic carbon-containing monocyclic ring containing 6 or 7 ring members, wherein one ring member is N, wherein R₇ is selected from —CH₃,—CH₂—CH₃, CH₂—CH₂—OH, CH₂—CHF₂, CH₂—CF₃, CH₂—CH₂-CH₂—OH, —CH (CH₃)₂, and —CH₂—(C═O)-NH₂, and wherein R₇ is bonded to A′ via a N ring member.

A′ can be a non-aromatic carbon-containing monocyclic ring containing 6 or 7 ring members, wherein one ring member is N, wherein R₇ is selected from —CH₃,—CH₂—CH₃, CH₂—CH₂—OH, CH₂—CHF₂, CH₂—CF₃, CH₂—CH₂-CH₂—OH, and —CH (CH₃)₂, and wherein R₇ is bonded to A′ via a N ring member.

A′ can be a non-aromatic carbon-containing monocyclic ring containing 6 or 7 ring members, wherein one ring member is N, wherein R₇ is selected from —CH₃,—CH₂—CH₃ and CH₂—CH₂—OH, and wherein R₇ is bonded to A′ via a N ring member.

A′ can be a non-aromatic carbon-containing monocyclic ring containing 6 ring members, wherein one ring member is N, wherein R₇ is selected from —CH₃,—CH₂—CH₃ and CH₂—CH₂—OH, and wherein R₇ is bonded to A′ via a N ring member.
R₁₀, R₁₁, and R₁₇ may be absent.
R₁₀ and R₁₁ may be absent.
R₂₄, R₂₅, R₂₆, and R₂₇ may be absent.
R₂₄, R₂₅, and R₂₆ may be absent.
R₁₀, R₁₁, R₁₇, R₂₄, R₂₅ and R₂₆, and R₂₇ may be absent.
R₁₀, R₁₁, R₂₄, R₂₅ and R₂₆ may be absent.
R₄₃ and R₄₄ may be absent.

A′ can be further substituted with at least one oxo substituent.

A′ can be further substituted with one oxo substituent.

In some embodiments, A′ is not substituted with further substituents beyond R₇.

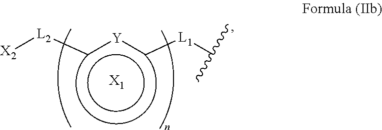

wherein:
n is 0 or 1;
L₁ is a linker selected from a single bond, —(CH₂)₁₋₃—, —(CH)(alkyl)-, and —C(alkyl)(alkyl)-; Y is selected from C, N, O and S;
X₁ is phenyl or a 5- or 6-membered carbon-containing aromatic ring containing one, two or three ring members that are selected from N, S, and O;
wherein X₁ may be optionally substituted with 1 or 2 substituents independently selected from alkyl, cycloalkyl, alkoxy, OH, O(cyclopropyl), CF₃, and halo;
L₂ is a linker selected from a single bond, —(CH₂)₁₋₃—, —(CH)(alkyl)-, —C(alkyl)(alkyl)-, —C(═O)—, and —SO₂—; and

X₂ is either:
(a) —NR₂OR₂₁, wherein R₂₀ is selected from H, alkyl, and —(CH₂)₀₋₃-R_{22;}
wherein R₂₁ is selected from H, alkyl, and —(CH₂)₀₋₃-R₂₃;

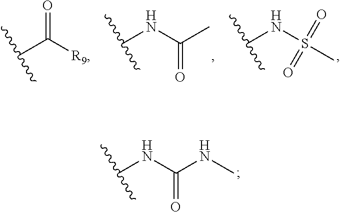

wherein R₂₂ is selected from cycloalkyl, heterocycloalkyl, aryl, heteroaryl,

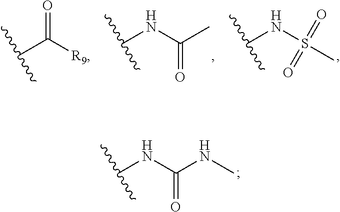

wherein R₂₃ is selected from cycloalkyl, heterocycloalkyl, aryl, heteroaryl, or
(b)₂-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected from alkyl, cycloalkyl, alkoxy, OH, Oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O, and X₂ may be further optionally substituted with 1 or 2 substituents R₂₉ and R₃₀, wherein R₂₉ and R₃₀ are independently selected from alkyl, cycloalkyl, alkoxy, OH, oxo, CF₃, and halo; or
(ii) Formula (III) comprising an aromatic ring A fused to a non-aromatic ring A′,

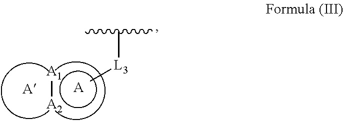

wherein:
L₃ is a single bond;
A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, O(cyclopropyl), CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R₇ is selected from alkyl, alkoxy, and —(CH₂)₀₋₃-R₈,

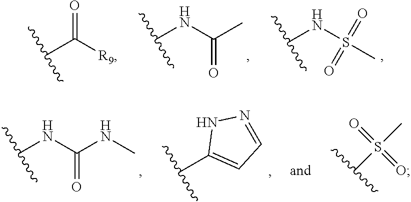

wherein R& is selected from cycloalkyl, heterocycloalkyl, wherein R₉ is selected from H, alkyl, OH, N(R₃₁)(R₃₂), and alkoxy;
wherein R₃₁ is H or small alkyl;
wherein R₃₂ is selected from H, small alkyl, and —(CH₂)₀₋₃heteroaryl;
wherein A′ may be optionally substituted with 1, 2, or 3 further substituents R₁₀, R₁₁, and R₁₇, wherein R₁₀, R₁₁, and R₁₇, are independently selected from alkyl, cycloalkyl, alkoxy, OH, oxo, CF₃, and halo, or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O, or wherein A′ may be optionally substituted with 1 or 2 further substituents R₄₃ and R₄₄ independently selected from alkyl, cycloalkyl, alkoxy, OH, oxo, CF₃, and halo, and two different carbon ring members within A′ are connected by a bridging —CH₂-group.
R₁ can be:
(i) Formula (IIb),

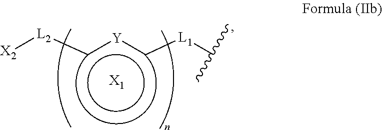

wherein:
n is 0 or 1;
L₁ is a linker selected from a single bond, —(CH₂)₁₋₃—, —(CH)(alkyl)-, and —C(alkyl)(alkyl)-; Y is selected from C, N, O and S;
X₁ is phenyl or a 5- or 6-membered carbon-containing aromatic ring containing one, two or three ring members that are selected from N, S, and O;
wherein X₁ may be optionally substituted with 1 or 2 substituents independently selected from alkyl, cycloalkyl, alkoxy, OH, CF₃, and halo;
L₂ is a linker selected from a single bond, —(CH₂)₁₋₃—, —(CH)(alkyl)-, —C(alkyl)(alkyl)-, —C(═O)—, and —SO₂—; and
X₂ is either:
(a) —NR₂OR₂₁, wherein R₂₀ is selected from H, alkyl, and —(CH₂)₀₋₃-R_{22;}
wherein R₂₁ is selected from H, alkyl, and —(CH₂)₀₋₃-R₂₃;

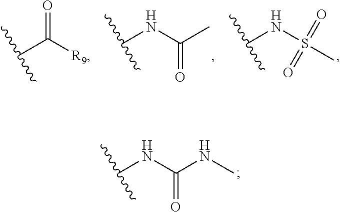

wherein R₂₂ is selected from cycloalkyl, heterocycloalkyl, aryl, heteroaryl,

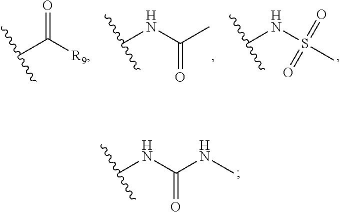

wherein R₂₃ is selected from cycloalkyl, heterocycloalkyl, aryl, heteroaryl, or
(b)₂-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O, and X₂ may be further optionally substituted with 1 or 2 substituents R₂₉ and R₃₀, wherein R₂₉ and R₃₀ are independently selected from alkyl, cycloalkyl, alkoxy, OH, oxo, CF₃, and halo; or
(ii) Formula (III) comprising an aromatic ring A fused to a non-aromatic ring A′,

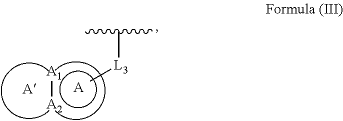

wherein:
L₃ is a single bond;
A₁ and A₂ are both carbon;
A is phenyl or a 5- or 6-membered carbon-containing aromatic ring containing one, two or three
wherein A may be optionally substituted with 1 or 2 substituents independently selected from alkyl, cycloalkyl, alkoxy, OH, CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R, is selected from alkyl, alkoxy, and —(CH₂)₀₋₃-R₈,

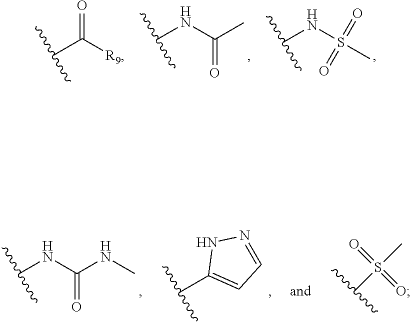

wherein R₈ is selected from cycloalkyl, heterocycloalkyl, wherein R₉ is selected from H, alkyl, OH, N(R₃₁)(R₃₂), and alkoxy;
wherein R₃₁ is H or small alkyl;
wherein R₃₂ is selected from H, small alkyl, and —(CH₂)₀₋₃heteroaryl;
wherein A′ may be optionally substituted with 1, 2, or 3 further substituents Rio, R₁₁, and R₁₇, wherein R₁₀, R₁₁, and R₁₇, are independently selected from alkyl, cycloalkyl, alkoxy, OH, oxo, CF₃, and halo, or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O.
R₁ can be:
(i) Formula (IIb),

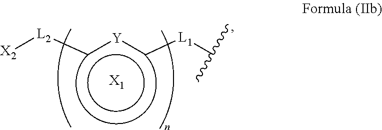

wherein:
n is 0 or 1;
L₁ is a linker selected from a single bond, —(CH₂)₁₋₃—, —(CH)(alkyl)-, and —C(alkyl)(alkyl)-; Y is selected from C, N, O and S;
X₁ is phenyl or a 5- or 6-membered carbon-containing aromatic ring containing one, two or three ring members that are selected from N, S, and O;
wherein X₁ may be optionally substituted with 1 or 2 substituents independently selected from alkyl, cycloalkyl, alkoxy, OH, CF₃, and halo;
L₂ is a linker selected from a single bond, —(CH₂)₁₋₃—, —(CH)(alkyl)-, —C(alkyl)(alkyl)-, —C(═O)—, and —SO₂—; and either:
wherein R₂₀ is selected from H, alkyl, and —(CH₂)₀₋₃-R_{22;}
wherein R₂₁ is selected from H, alkyl, and —(CH₂)₀₋₃-R_{23;}
X₂ is (a) —NR₂OR₂₁,

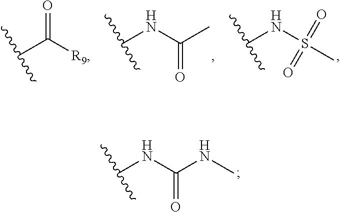

wherein R₂₂ is selected from cycloalkyl, heterocycloalkyl, aryl, heteroaryl,

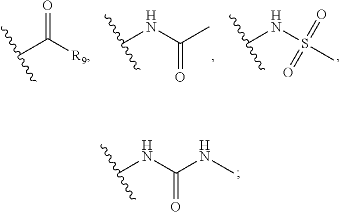

wherein R₂₃ is selected from cycloalkyl, heterocycloalkyl, aryl, heteroaryl, or
(b)₂-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected from alkoxy, oxo, alkyl, cycloalkyl, OH, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O; or

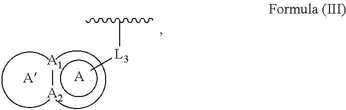

wherein:
L₃ is a single bond;
A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R₇ is selected from alkyl, alkoxy, and —(CH₂)₀₋₃-R₈,

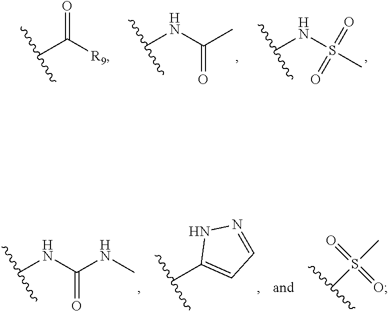

wherein R₈ is selected from cycloalkyl, heterocycloalkyl, wherein R₉ is selected from H, alkyl, OH, NH₂, and alkoxy;
wherein A′ may be optionally substituted with 1, 2, or 3 further substituents R₁₀, R₁₁, and R₁₇, wherein R₁₀, R₁₁, and R₁₇, are independently selected from alkyl, cycloalkyl, alkoxy, OH, oxo, CF₃, and halo, or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O.
R₁ can be:
(i) Formula (IIa),

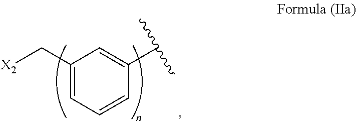

wherein:
n is 0 or 1;
X₂ is a non-aromatic, carbon-containing monocyclic ring containing 5, 6, or 7 ring members, wherein at least one ring member is independently selected from N, S, and O;
wherein X₂ may be optionally substituted with 1 or 2 substituents R₁₈ and R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O; or
(ii) Formula (III) comprising an aromatic ring A fused to a non-aromatic ring A′,

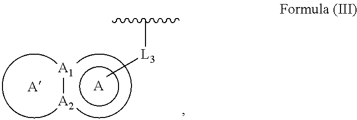

wherein:
L₃ is a single bond;
A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R₇ is selected from alkyl, alkoxy, and —(CH₂)₀₋₃-R₈,

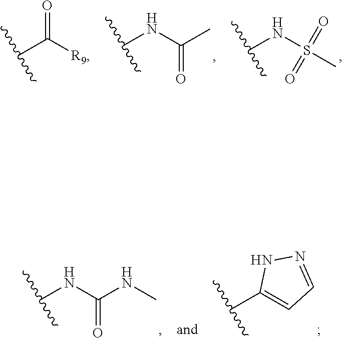

wherein R₈ is selected from cycloalkyl, heterocycloalkyl, wherein R₉ is selected from H, alkyl, OH, NH₂, and alkoxy;
wherein A′ may be optionally substituted with 1 or 2 further substituents R₁₀ and R₁₁, wherein R₁₀ and R₁₁ are independently selected from alkyl, cycloalkyl, alkoxy, OH, oxo, CF₃, and halo, or wherein A′ may be optionally substituted with 1 further substituent R₂₄ selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O.

Preferably, R₁ can be:
(i) Formula (IIb),

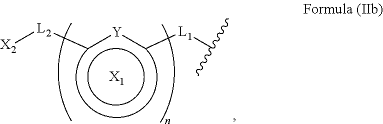

wherein:
n is 1;
L₁ is a linker selected from a single bond, —(CH₂)₁₋₃—, —(CH)(alkyl)-, and —C(alkyl)(alkyl)-; Y is selected from C, N, O and S;
X₁ is phenyl or a 5- or 6-membered carbon-containing aromatic ring containing one, two or three ring members that are selected from N, S, and O;
wherein X₁ may be optionally substituted with 1 or 2 substituents independently selected from alkyl, cycloalkyl, alkoxy, OH, O(cyclopropyl), CF₃, and halo;
L₂ is a linker selected from a single bond, —(CH₂)₁₋₃—, —(CH)(alkyl)-, —C(alkyl)(alkyl)-, —C(═O)—, and —SO₂—; and

X₂ is either:
(a) —NR₂OR₂₁, wherein R₂₀ is selected from H, alkyl, and —(CH₂)₀₋₃-R_{22;}
wherein R₂₁ is selected from H, alkyl, and —(CH₂)₀₋₃-R₂₃;

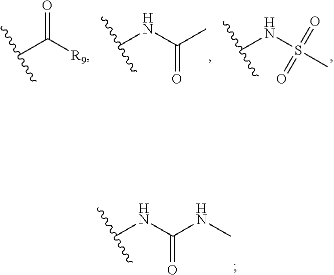

wherein R₂₂ is selected from cycloalkyl, heterocycloalkyl, aryl, heteroaryl,

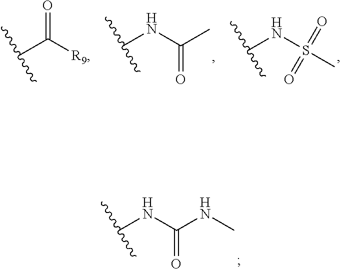

wherein R₂₃ is selected from cycloalkyl, heterocycloalkyl, aryl, heteroaryl, or
(b)₂-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected from alkoxy, OH, alkyl, cycloalkyl, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O, and X₂ may be further optionally substituted with 1 or 2 substituents R₂₉ and R₃₀, wherein R₂₉ and R₃₀ are independently selected from alkyl, cycloalkyl, alkoxy, OH, oxo, CF₃, and halo; or
(ii) Formula (III) comprising an aromatic ring A fused to a non-aromatic ring A′,

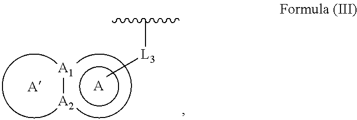

wherein:
L₃ is a single bond;
A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, O(cyclopropyl), CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R₇ is selected from alkyl, alkoxy, and —(CH₂)₀₋₃-R₈,

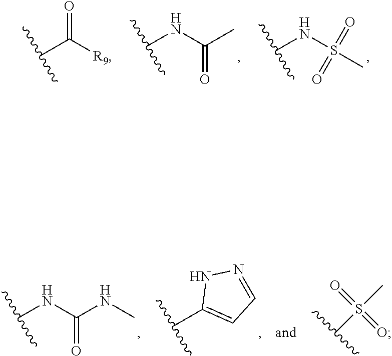

wherein R₈ is selected from cycloalkyl, heterocycloalkyl, wherein R₉ is selected from H, alkyl, OH, N(R₃₁)(R₃₂), and alkoxy;
wherein R₃₁ is H or small alkyl;
wherein R₃₂ is selected from H, small alkyl, and —(CH₂)₀₋₃heteroaryl;
wherein A′ may be optionally substituted with 1, 2, or 3 further substituents Rio, R₁₁, and R₁₇, wherein R₁₀, R₁₁, and R₁₇, are independently selected from alkyl, cycloalkyl, alkoxy, OH, oxo, CF₃, and halo, or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O, or wherein A′ may be optionally substituted with 1 or 2 further substituents R₄₃ and R₄₄ independently selected from alkyl, cycloalkyl, alkoxy, OH, oxo, CF₃, and halo, and two different carbon ring members within A′ are connected by a bridging —CH₂-group.
R₁ can be:
(i) Formula (IIb),

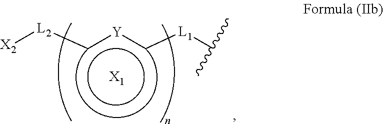

wherein:
n is 1;
L₁ is a linker selected from a single bond, —(CH₂)₁₋₃—, —(CH)(alkyl)-, and —C(alkyl)(alkyl); Y is selected from C, N, O and S;
X₁ is phenyl or a 5- or 6-membered carbon-containing aromatic ring containing one, two or three ring members that are selected from N, S, and O;
wherein X₁ may be optionally substituted with 1 or 2 substituents independently selected from alkyl, cycloalkyl, alkoxy, OH, CF₃, and halo;
L₂ is a linker selected from a single bond, —(CH₂)₁₋₃—, —(CH)(alkyl)-, —C(alkyl)(alkyl)-, —C(═O)—, and —SO₂—; and either:
wherein R₂₀ is selected from H, alkyl, and —(CH₂)₀₋₃-R_{22;}
wherein R₂₁ is selected from H, alkyl, and —(CH₂)₀₋₃-R_{23;}
X₂ is (a) —NR₂OR₂₁,

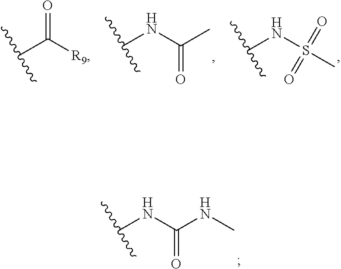

wherein R₂₂ is selected from cycloalkyl, heterocycloalkyl, aryl, heteroaryl,

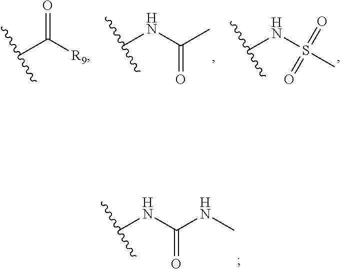

wherein R₂₃ is selected from cycloalkyl, heterocycloalkyl, aryl, heteroaryl, or
(b)₂-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O, and X₂ may be further optionally substituted with 1 or 2 substituents R₂₉ and R₃₀, wherein R₂₉ and R₃₀ are independently selected from alkyl, cycloalkyl, alkoxy, OH, oxo, CF₃, and halo; or

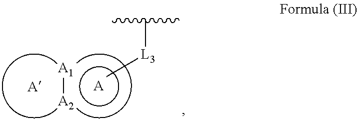

wherein:
L₃ is a single bond;
A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R₇ is selected from alkyl, alkoxy, and —(CH₂)₀₋₃-R₈,

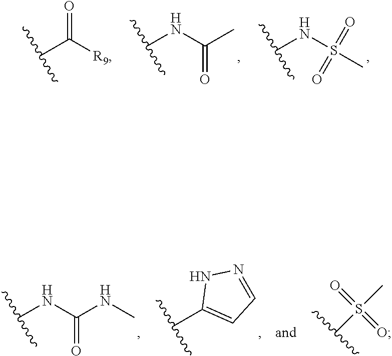

wherein R₈ is selected from cycloalkyl, heterocycloalkyl, wherein R₉ is selected from H, alkyl, OH, N(R₃₁)(R₃₂), and alkoxy;
wherein R₃₁ is H or small alkyl;
wherein R₃₂ is selected from H, small alkyl, and —(CH₂)₀₋₃heteroaryl;
wherein A′ may be optionally substituted with 1, 2, or 3 further substituents R₁₀, R₁₁, and R₁₇, wherein R₁₀, R₁₁, and R₁₇, are independently selected from alkyl, cycloalkyl, alkoxy, OH, oxo, CF₃, and halo, or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O.
R₁ can be:
(i) Formula (IIb),

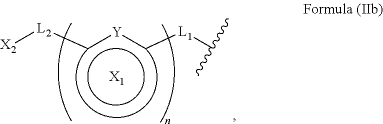

wherein:
n is 1;
L₁ is a linker selected from a single bond, —(CH₂)₁₋₃—, —(CH)(alkyl)-, and —C(alkyl)(alkyl)-; Y is selected from C, N, O and S;
X₁ is phenyl or a 5- or 6-membered carbon-containing aromatic ring containing one, two or three ring members that are selected from N, S, and O;
wherein X₁ may be optionally substituted with 1 or 2 substituents independently selected from alkyl, cycloalkyl, alkoxy, OH, CF₃, and halo;
L₂ is a linker selected from a single bond, —(CH₂)₁₋₃—, —(CH)(alkyl)-, —C(alkyl)(alkyl)-, —C(═O)—, and —SO₂—; and

X₂ is either:
(a) —NR₂OR₂₁, wherein R₂₀ is selected from H, alkyl, and —(CH₂)₀₋₃-R_{22;}
wherein R₂₁ is selected from H, alkyl, and —(CH₂)₀₋₃-R₂₃;

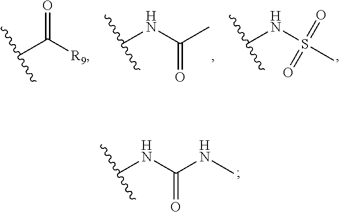

wherein R₂₂ is selected from cycloalkyl, heterocycloalkyl, aryl, heteroaryl,

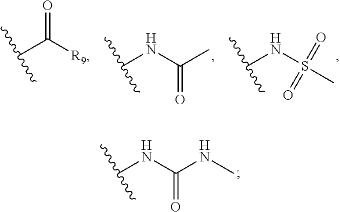

wherein R₂₃ is selected from cycloalkyl, heterocycloalkyl, aryl, heteroaryl, or
(b)₂-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O; or
(ii) Formula (III) comprising an aromatic ring A fused to a non-aromatic ring A′,

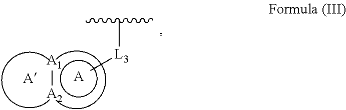

wherein:
L₃ is a single bond;
A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R₇ is selected from alkyl, alkoxy, and —(CH₂)₀₋₃-R₈,

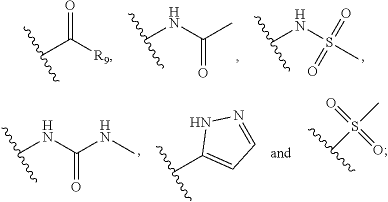

wherein R₈ is selected from cycloalkyl, heterocycloalkyl, wherein R₉ is selected from H, alkyl, OH, NH₂, and alkoxy;
wherein A′ may be optionally substituted with 1, 2, or 3 further substituents R₁₀, R₁₁, and R₁₇, wherein R₁₀, R₁₁, and R₁₇, are independently selected from alkyl, cycloalkyl, alkoxy, OH, oxo, CF₃, and halo, or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O.
R₁ can be:
(i) Formula (IIa),

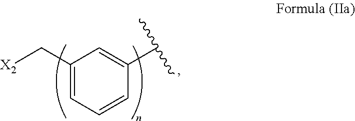

wherein:

nis 1;
X₂ is a non-aromatic, carbon-containing mono- or bi-cyclic ring containing 5, 6, or 7 ring members, wherein at least one ring member is independently selected from N, S, and O;
wherein X₂ may be optionally substituted with 1 or 2 substituents R₁₈ and R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O; or
(ii) Formula (III) comprising an aromatic ring A fused to a non-aromatic ring A′,

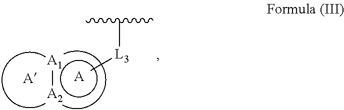

wherein:
L₃ is a single bond;
A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R₇ is selected from alkyl, alkoxy, and —(CH₂)₀₋₃-R₈,

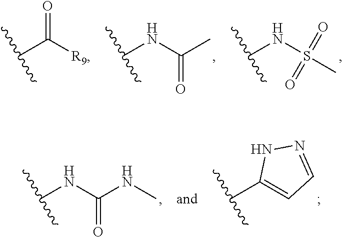

wherein R₈ is selected from cycloalkyl, heterocycloalkyl, wherein R₈ is selected from H, alkyl, OH, NH₂, and alkoxy;
wherein A′ may be optionally substituted with 1 or 2 further substituents R₁₀ and R₁₁, wherein R₁₀ and R₁₁ are independently selected from alkyl, cycloalkyl, alkoxy, OH, oxo, CF₃, and halo, or wherein A′ may be optionally substituted with 1 further substituent R₂₄ selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O.

Formula (II) can be represented by Formula (IIb),

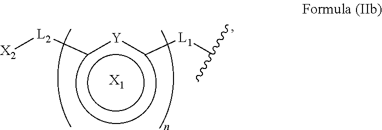

wherein:
n is 0 or 1;
L₁ is a linker selected from a single bond, —(CH₂)₁₋₃—, —(CH)(alkyl)-, and —C(alkyl)(alkyl)-;
Y is selected from C, N, O and S;
X₁ is phenyl or a 5- or 6-membered carbon-containing aromatic ring containing one, two or three ring members that are selected from N, S, and O;
wherein X₁ may be optionally substituted with 1 or 2 substituents independently selected from alkyl, cycloalkyl, alkoxy, OH, O(cyclopropyl), CF₃, and halo;
L₂ is a linker selected from a single bond, —(CH₂)₁₋₃—, —(CH)(alkyl)-, —C(alkyl)(alkyl)-, —C(═O)—, and —SO₂—; and X₂ is either:
(a) —NR₂OR₂₁, wherein R₂₀ is selected from H, alkyl, and —(CH₂)₀₋₃-R_{22;}
wherein R₂₁ is selected from H, alkyl, and —(CH₂)₀₋₃-R₂₃;

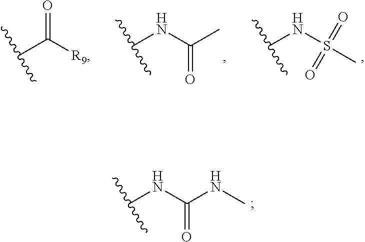

wherein R₂₂ is selected from cycloalkyl, heterocycloalkyl, aryl, heteroaryl,

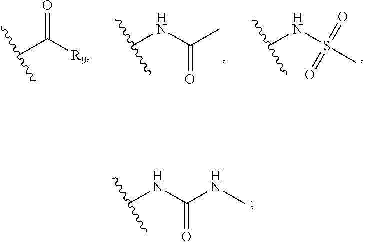

wherein R₂₃ is selected from cycloalkyl, heterocycloalkyl, aryl, heteroaryl, or
(b)₂-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected from alkyl, cycloalkyl, alkoxy, OH, Oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O, and X₂ may be further optionally substituted with 1 or 2 substituents R₂₉ and R₃₀, wherein R₂₉ and R₃₀ are independently selected from alkyl, cycloalkyl, alkoxy, OH, oxo, CF₃, and halo.

Formula (II) can be represented by Formula (IIb),

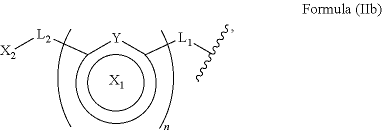

wherein:
n is 0 or 1;
L₁ is a linker selected from a single bond, —(CH₂)₁₋₃—, —(CH)(alkyl)-, and —C(alkyl)(alkyl)-; Y is selected from C, N, O and S;
X₁ is phenyl or a 5- or 6-membered carbon-containing aromatic ring containing one, two or three ring members that are selected from N, S, and O;
wherein X₁ may be optionally substituted with 1 or 2 substituents independently selected from alkyl, cycloalkyl, alkoxy, OH, CF₃, and halo; L₂ is a linker selected from a single bond, —(CH₂)₁₋₃—, —(CH)(alkyl)-, —C(alkyl)(alkyl)-, —C(═O)—, and —SO₂—; and X₂ is either:
(a) —NR₂OR₂₁, wherein R₂₀ is selected from H, alkyl, and —(CH₂)₀₋₃-R_{22;}
wherein R₂₁ is selected from H, alkyl, and —(CH₂)₀₋₃-R₂₃;

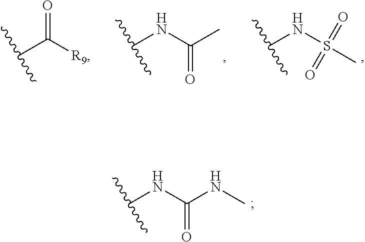

wherein R₂₂ is selected from cycloalkyl, heterocycloalkyl, aryl, heteroaryl,

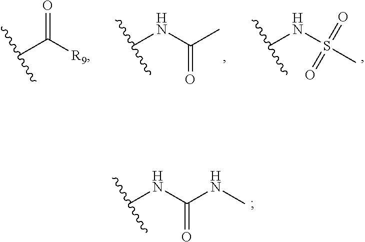

wherein R₂₃ is selected from cycloalkyl, heterocycloalkyl, aryl, heteroaryl, or
(b)₂-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O, and X₂ may be further optionally substituted with 1 or 2 substituents R₂₉ and R₃₀, wherein R₂₉ and R₃₀ are independently selected from alkyl, cycloalkyl, alkoxy, OH, oxo, CF₃, and halo.

Formula (II) can be represented by Formula (IIb),

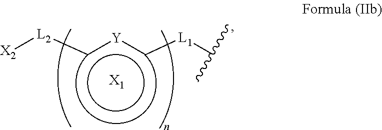

wherein:
n is 0 or 1;
L₁ is a linker selected from a single bond, —(CH₂)₁₋₃—, —(CH)(alkyl)-, and —C(alkyl)(alkyl); Y is selected from C, N, O and S;
X₁ is phenyl or a 5- or 6-membered carbon-containing aromatic ring containing one, two or three ring members that are selected from N, S, and O;
wherein X₁ may be optionally substituted with 1 or 2 substituents independently selected from alkyl, cycloalkyl, alkoxy, OH, CF₃, and halo; L₂ is a linker selected from a single bond, —(CH₂)₁₋₃—, —(CH)(alkyl)-, —C(alkyl)(alkyl)-, —C(═O)—, and —SO₂—; and

X₂ is either:
(a) —NR₂OR₂₁, wherein R₂₀ is selected from H, alkyl, and —(CH₂)₀₋₃-R_{22;}
wherein R₂₁ is selected from H, alkyl, and —(CH₂)₀₋₃-R₂₃;

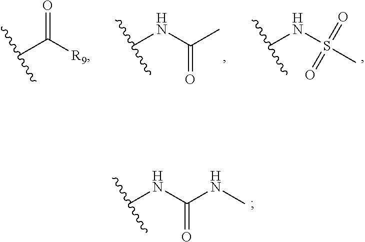

wherein R₂₂ is selected from cycloalkyl, heterocycloalkyl, aryl, heteroaryl,

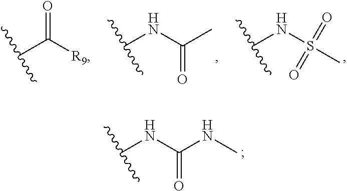

wherein R₂₃ is selected from cycloalkyl, heterocycloalkyl, aryl, heteroaryl, or
(b)₂-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O.

Formula (II) can be represented by Formula (IIa),

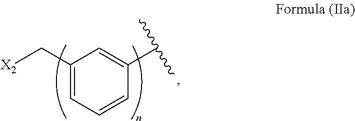

wherein:
n is 0 or 1; X₂ is a non-aromatic, carbon-containing mono- or bi-cyclic ring containing 5, 6, or 7 ring members, wherein at least one ring member is independently selected from N, S, and O;
wherein X₂ may be optionally substituted with 1 or 2 substituents R₁₈ and R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O.

Formula (II) can be represented by Formula (IIb),

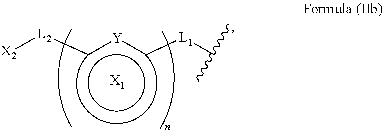

wherein:
n is 1; L₁ is a linker selected from a single bond, —(CH₂)₁₋₃—, —(CH)(alkyl)-, and —C(alkyl)(alkyl)-; Y is selected from C, N, O and S;
X₁ is phenyl or a 5- or 6-membered carbon-containing aromatic ring containing one, two or three ring members that are selected from N, S, and O;
wherein X₁ may be optionally substituted with 1 or 2 substituents independently selected from alkyl, cycloalkyl, alkoxy, OH, O(cyclopropyl), CF₃, and halo; L₂ is a linker selected from a single bond, —(CH₂)₁₋₃—, —(CH)(alkyl)-, —C(alkyl)(alkyl)-, —C(═O)—, and —SO₂—; and X₂ is either:
(a) —NR₂OR₂₁, wherein R₂₀ is selected from H, alkyl, and —(CH₂)₀₋₃-R_{22;}
wherein R₂₁ is selected from H, alkyl, and —(CH₂)₀₋₃-R₂₃;

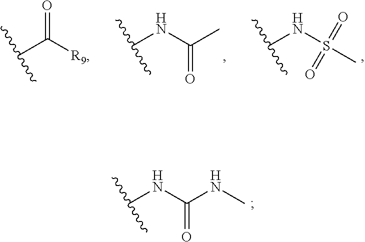

wherein R₂₂ is selected from cycloalkyl, heterocycloalkyl, aryl, heteroaryl,

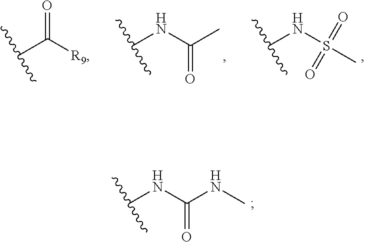

wherein R₂₃ is selected from cycloalkyl, heterocycloalkyl, aryl, heteroaryl, or
(b)₂-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O, and X₂ may be further optionally substituted with 1 or 2 substituents R₂₉ and R₃₀, wherein R₂₉ and R₃₀ are independently selected from alkyl, cycloalkyl, alkoxy, OH, oxo, CF₃, and halo.

Formula (II) can be represented by Formula (IIb),

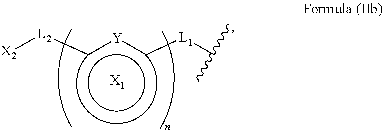

wherein:
n is 1;
L₁ is a linker selected from a single bond, —(CH₂)₁₋₃—, —(CH)(alkyl)-, and —C(alkyl)(alkyl); Y is selected from C, N, O and S;
X₁ is phenyl or a 5- or 6-membered carbon-containing aromatic ring containing one, two or three ring members that are selected from N, S, and O;
wherein X₁ may be optionally substituted with 1 or 2 substituents independently selected from alkyl, cycloalkyl, alkoxy, OH, CF₃, and halo; L₂ is a linker selected from a single bond, —(CH₂)₁₋₃—, —(CH)(alkyl)-, —C(alkyl)(alkyl)-, —C(═O)—, and —SO₂—; and

X₂ is either:
(a) —NR₂OR₂₁, wherein R₂₀ is selected from H, alkyl, and —(CH₂)₀₋₃-R_{22;}
wherein R₂₁ is selected from H, alkyl, and —(CH₂)₀₋₃-R₂₃;

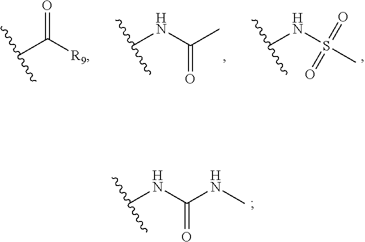

wherein R₂₂ is selected from cycloalkyl, heterocycloalkyl, aryl, heteroaryl,

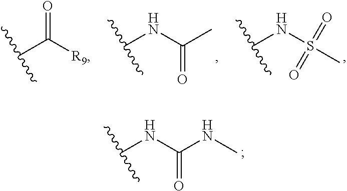

wherein R₂₃ is selected from cycloalkyl, heterocycloalkyl, aryl, heteroaryl, or
(b)₂-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O, and X₂ may be further optionally substituted with 1 or 2 substituents R₂₉ and R₃₀, wherein R₂₉ and R₃₀ are independently selected from alkyl, cycloalkyl, alkoxy, OH, oxo, CF₃, and halo.

Formula (II) can be represented by Formula (IIb),

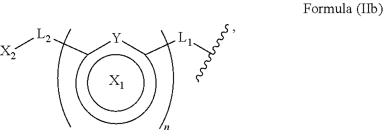

wherein:
n is 1;
L₁ is a linker selected from a single bond, —(CH₂)₁₋₃—, —(CH)(alkyl)-, and —C(alkyl)(alkyl)-; Y is selected from C, N, O and S; X₁ is phenyl or a 5- or 6-membered carbon-containing aromatic ring containing one, two or three ring members that are selected from N, S, and O;
wherein X₁ may be optionally substituted with 1 or 2 substituents independently selected from alkyl, cycloalkyl, alkoxy, OH, CF₃, and halo; L₂ is a linker selected from a single bond, —(CH₂)₁₋₃—, —(CH)(alkyl)-, —C(alkyl)(alkyl)-, —C(═O)—, and —SO₂—; and

X₂ is either:
(a) —NR₂OR₂₁, wherein R₂₀ is selected from H, alkyl, and —(CH₂)₀₋₃-R_{22;}
wherein R₂₁ is selected from H, alkyl, and —(CH₂)₀₋₃-R₂₃;

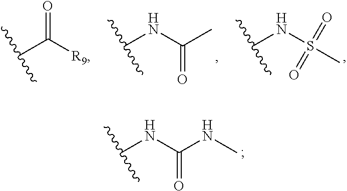

wherein R₂₂ is selected from cycloalkyl, heterocycloalkyl, aryl, heteroaryl,

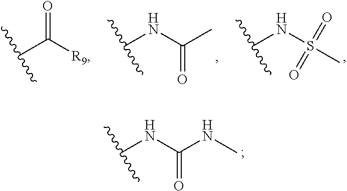

wherein R₂₃ is selected from cycloalkyl, heterocycloalkyl, aryl, heteroaryl, or
(b)₂-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected from alkyl, cycloalkyl, alkoxy, OH, Oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O.

Formula (II) can be represented by Formula (IIa),

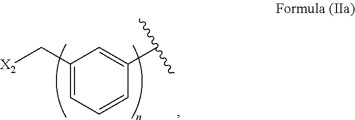

wherein:
n is 1;

X₂ is a non-aromatic, carbon-containing mono- or bi-cyclic ring containing 5, 6, or 7 ring members, wherein at least one ring member is independently selected from N, S, and O;
wherein X₂ may be optionally substituted with 1 or 2 substituents R₁₈ and R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O.
R₁ can be Formula (IIb),

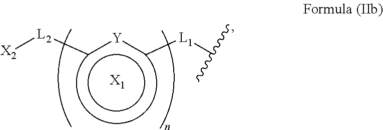

wherein:
n is 0 or 1;
L₁ is a linker selected from a single bond, —(CH₂)₁₋₃—, —(CH)(alkyl)-, and —C(alkyl)(alkyl)-; Y is selected from C, N, O and S; X₁ is phenyl or a 5- or 6-membered carbon-containing aromatic ring containing one, two or three ring members that are selected from N, S, and O;
wherein X₁ may be optionally substituted with 1 or 2 substituents independently selected from alkyl, cycloalkyl, alkoxy, OH, O(cyclopropyl), CF₃, and halo; L₂ is a linker selected from a single bond, —(CH₂)₁₋₃—, —(CH)(alkyl)-, —C(alkyl)(alkyl)-, —C(═O)—, and —SO₂—; and X₂ is either:
(a) —NR₂OR₂₁, wherein R₂₀ is selected from H, alkyl, and —(CH₂)₀₋₃-R_{22;}
wherein R₂₁ is selected from H, alkyl, and —(CH₂)₀₋₃-R₂₃;

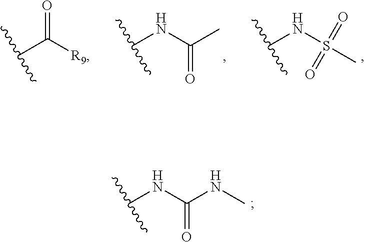

wherein R₂₂ is selected from cycloalkyl, heterocycloalkyl, aryl, heteroaryl,

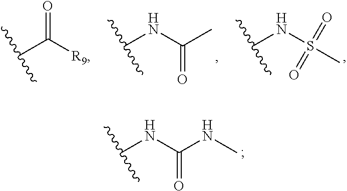

wherein R₂₃ is selected from cycloalkyl, heterocycloalkyl, aryl, heteroaryl, or
(b)₂-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O, and X₂ may be further optionally substituted with 1 or 2 substituents R₂₉ and R₃₀, wherein R₂₉ and R₃₀ are independently selected from alkyl, cycloalkyl, alkoxy,

OH, oxo, CF₃, and halo.
R₁ can be Formula (IIb),

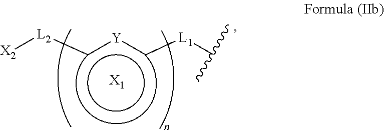

wherein:
n is 0 or 1;
L₁ is a linker selected from a single bond, —(CH₂)₁₋₃—, —(CH)(alkyl)-, and —C(alkyl)(alkyl)-;
Y is selected from C, N, O and S;
X₁ is phenyl or a 5- or 6-membered carbon-containing aromatic ring containing one, two or three ring members that are selected from N, S, and O;
wherein X₁ may be optionally substituted with 1 or 2 substituents independently selected from alkyl, cycloalkyl, alkoxy, OH, CF₃, and halo;
L₂ is a linker selected from a single bond, —(CH₂)₁₋₃—, —(CH)(alkyl)-, —C(alkyl)(alkyl)-, —C(═O)—, and —SO₂—; and

X₂ is either:
(a) —NR₂OR₂₁, wherein R₂₀ is selected from H, alkyl, and —(CH₂)₀₋₃-R_{22;}
wherein R₂₁ is selected from H, alkyl, and —(CH₂)₀₋₃-R₂₃;

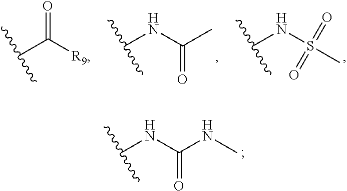

wherein R₂₂ is selected from cycloalkyl, heterocycloalkyl, aryl, heteroaryl,

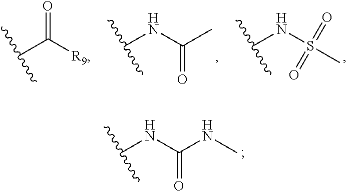

wherein R₂₃ is selected from cycloalkyl, heterocycloalkyl, aryl, heteroaryl, or
(b)₂-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected from alkyl, cycloalkyl, alkoxy, OH, OXO, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O, and X₂ may be further optionally substituted with 1 or 2 substituents R₂₉ and R₃₀, wherein R₂₉ and R₃₀ are independently selected from alkyl, cycloalkyl, alkoxy, OH, oxo, CF₃, and halo.
R₁ can be Formula (IIb),

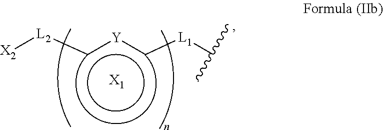

wherein:
n is 0 or 1;
L₁ is a linker selected from a single bond, —(CH₂)₁₋₃—, —(CH)(alkyl)-, and —C(alkyl)(alkyl)-;
Y is selected from C, N, O and S;
X₁ is phenyl or a 5- or 6-membered carbon-containing aromatic ring containing one, two or three ring members that are selected from N, S, and O;
wherein X₁ may be optionally substituted with 1 or 2 substituents independently selected from alkyl, cycloalkyl, alkoxy, OH, CF₃, and halo;
L₂ is a linker selected from a single bond, —(CH₂)₁₋₃—, —(CH)(alkyl)-, —C(alkyl)(alkyl)-, —C(═O)—, and —SO₂—; and

X₂ is either:
(a) —NR₂OR₂₁, wherein R₂₀ is selected from H, alkyl, and —(CH₂)₀₋₃-R_{22;}
wherein R₂₁ is selected from H, alkyl, and —(CH₂)₀₋₃-R₂₃;

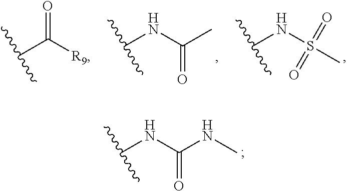

wherein R₂₂ is selected from cycloalkyl, heterocycloalkyl, aryl, heteroaryl,

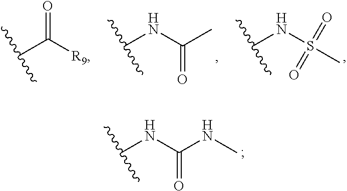

wherein R₂₃ is selected from cycloalkyl, heterocycloalkyl, aryl, heteroaryl, or
(b)₂-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O.
R₁ can be Formula (IIa),

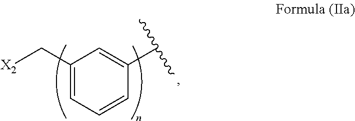

wherein:
n is 0 or 1;

X₂ is a non-aromatic, carbon-containing monocyclic ring containing 5, 6, or 7 ring members, wherein at least one ring member is independently selected from N, S, and O;
wherein X₂ may be optionally substituted with 1 or 2 substituents R₁₈ and R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O.
R₁ can be Formula (IIb),

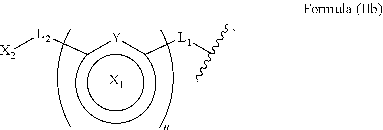

wherein:
n is 1;
L₁ is a linker selected from a single bond, —(CH₂)₁₋₃—, —(CH)(alkyl)-, and —C(alkyl)(alkyl)-; Y is selected from C, N, O and S; X₁ is phenyl or a 5- or 6-membered carbon-containing aromatic ring containing one, two or three ring members that are selected from N, S, and O;
wherein X₁ may be optionally substituted with 1 or 2 substituents independently selected from alkyl, cycloalkyl, alkoxy, OH, O(cyclopropyl), CF₃, and halo; L₂ is a linker selected from a single bond, —(CH₂)₁₋₃—, —(CH)(alkyl)-, —C(alkyl)(alkyl)-, —C(═O)—, and —SO₂—; and X₂ is either:
(a) —NR₂OR₂₁, wherein R₂₀ is selected from H, alkyl, and —(CH₂)₀₋₃-R_{22;}
wherein R₂₁ is selected from H, alkyl, and —(CH₂)₀₋₃-R₂₃;

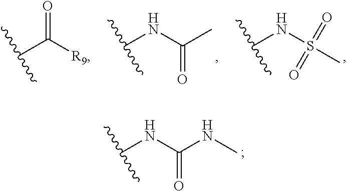

wherein R₂₂ is selected from cycloalkyl, heterocycloalkyl, aryl, heteroaryl,

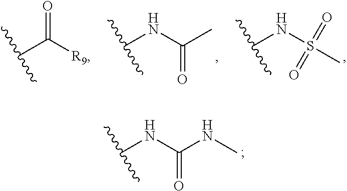

wherein R₂₃ is selected from cycloalkyl, heterocycloalkyl, aryl, heteroaryl, or
(b)₂-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O, and X₂ may be further optionally substituted with 1 or 2 substituents R₂₉ and R₃₀, wherein R₂₉ and R₃₀ are independently selected from alkyl, cycloalkyl, alkoxy, OH, oxo, CF₃, and halo.
R₁ can be Formula (IIb),

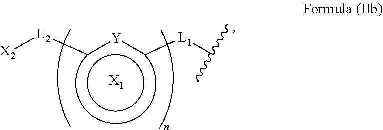

wherein:
n is 1;
L₁ is a linker selected from a single bond, —(CH₂)₁₋₃—, —(CH)(alkyl)-, and —C(alkyl)(alkyl)-; Y is selected from C, N, O and S; X₁ is phenyl or a 5- or 6-membered carbon-containing aromatic ring containing one, two or three ring members that are selected from N, S, and O;
wherein X₁ may be optionally substituted with 1 or 2 substituents independently selected from alkyl, cycloalkyl, alkoxy, OH, CF₃, and halo; L₂ is a linker selected from a single bond, —(CH₂)₁₋₃—, —(CH)(alkyl)-, —C(alkyl)(alkyl)-, —C(═O)—, and —SO₂—; and
X₂ is either:
(a) —NR₂OR₂₁,
wherein R₂₀ is selected from H, alkyl, and —(CH₂)₀₋₃-R_{22;}
wherein R₂₁ is selected from H, alkyl, and —(CH₂)₀₋₃-R₂₃;

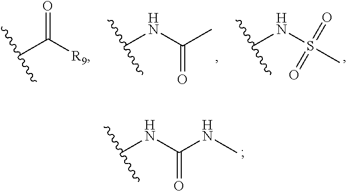

wherein R₂₂ is selected from cycloalkyl, heterocycloalkyl, aryl, heteroaryl,

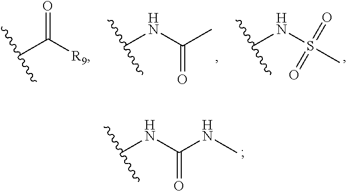

wherein R₂₃ is selected from cycloalkyl, heterocycloalkyl, aryl, heteroaryl, or
(b)₂-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected from alkyl, cycloalkyl, alkoxy, OH, Oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O, and X₂ may be further optionally substituted with 1 or 2 substituents R₂₉ and R₃₀, wherein R₂₉ and R₃₀ are independently selected from alkyl, cycloalkyl, alkoxy, OH, oxo, CF₃, and halo.
R₁ can be Formula (IIb),

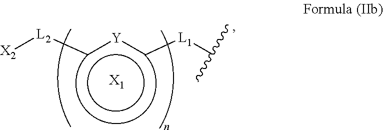

wherein:
n is 1;
L₁ is a linker selected from a single bond, —(CH₂)₁₋₃—, —(CH)(alkyl)-, and —C(alkyl)(alkyl)-; Y is selected from C, N, O and S;
X₁ is phenyl or a 5- or 6-membered carbon-containing aromatic ring containing one, two or three ring members that are selected from N, S, and O;
wherein X₁ may be optionally substituted with 1 or 2 substituents independently selected from alkyl, cycloalkyl, alkoxy, OH, CF₃, and halo;
L₂ is a linker selected from a single bond, —(CH₂)₁₋₃—, —(CH)(alkyl)-, —C(alkyl)(alkyl)-, —C(═O)—, and —SO₂—; and
X₂ is either:
(a) —NR₂OR₂₁, wherein R₂₀ is selected from H, alkyl, and —(CH₂)₀₋₃-R_{22;}
wherein R₂₁ is selected from H, alkyl, and —(CH₂)₀₋₃-R₂₃;

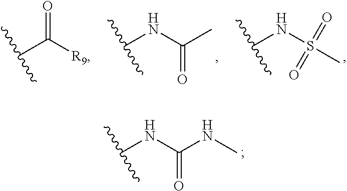

wherein R₂₂ is selected from cycloalkyl, heterocycloalkyl, aryl, heteroaryl,

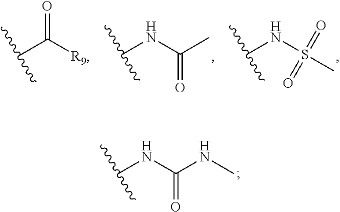

wherein R₂₃ is selected from cycloalkyl, heterocycloalkyl, aryl, heteroaryl, or
(b)₂-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected from alkyl, cycloalkyl, alkoxy, OH, OXO, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O.
R₁ can be Formula (IIa),

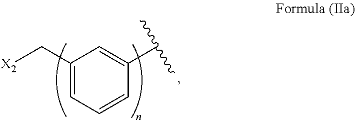

wherein:
n is 1;
X₂ is a non-aromatic, carbon-containing mono- or bi-cyclic ring containing 5, 6, or 7 ring members, wherein at least one ring member is independently selected from N, S, and O;
wherein X₂ may be optionally substituted with 1 or 2 substituents R₁₈ and R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O.

Formula (III) can comprise an aromatic ring A fused to a non-aromatic ring A′,

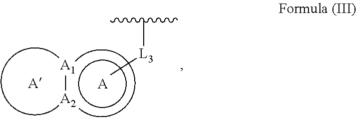

wherein:
L₃ is a single bond;
A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, O(cyclopropyl), CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R₇ is selected from alkyl, alkoxy, and —(CH₂)₀₋₃-R₈,

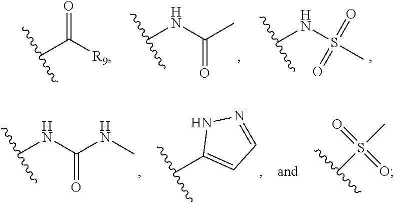

wherein R₈ is selected from cycloalkyl, heterocycloalkyl, wherein R₉ is selected from H, alkyl, OH, N(R₃₁)(R₃₂), and alkoxy;
wherein R₃₁ is H or small alkyl;
wherein R₃₂ is selected from H, small alkyl, and —(CH₂)₀₋₃heteroaryl;
wherein A′ may be optionally substituted with 1, 2, or 3 further substituents R₁₀, R₁₁, and R₁₇, wherein R₁₀, R₁₁, and R₁₇, are independently selected from alkyl, cycloalkyl, alkoxy, OH, oxo, CF₃, and halo, or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O, or wherein A′ may be optionally substituted with 1 or 2 further substituents R₄₃ and R₄₄ independently selected from alkyl, cycloalkyl, alkoxy, OH, oxo, CF₃, and halo, and two different carbon ring members within A′ are connected by a bridging —CH₂-group.

Formula (III) can comprise an aromatic ring A fused to a non-aromatic ring A′,

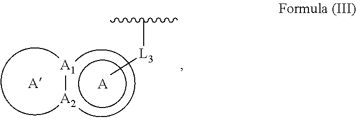

wherein:
L₃ is a single bond;
A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R₇ is selected from alkyl, alkoxy, and

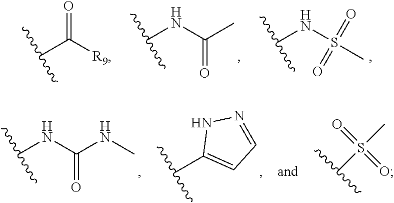

wherein R₈ is selected from cycloalkyl, heterocycloalkyl, wherein R₉ is selected from H, alkyl, OH, N(R₃₁)(R₃₂), and alkoxy;
wherein R₃₁ is H or small alkyl;
wherein R₃₂ is selected from H, small alkyl, and —(CH₂)₀₋₃heteroaryl;
wherein A′ may be optionally substituted with 1, 2, or 3 further substituents R₁₀, R₁₁, and R₁₇, wherein R₁₀, R₁₁, and R₁₇, are independently selected from alkyl, cycloalkyl, alkoxy, OH, oxo, CF₃, and halo, or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O.

Formula (III) can comprise an aromatic ring A fused to a non-aromatic ring A′,

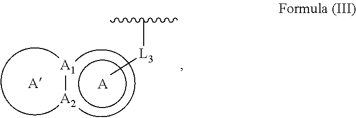

wherein:
L₃ is a single bond;
A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R₇ is selected from alkyl, alkoxy, and

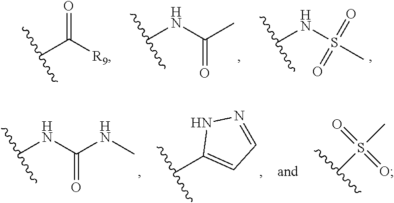

wherein R₈ is selected from cycloalkyl, heterocycloalkyl, wherein R₉ is selected from H, alkyl, OH, NH₂, and alkoxy;
wherein A′ may be optionally substituted with 1, 2, or 3 further substituents R₁₀, R₁₁, and R₁₇, wherein R₁₀, R₁₁, and R₁₇, are independently selected from alkyl, cycloalkyl, alkoxy, OH, oxo, CF₃, and halo, or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O.

Formula (III) can comprise an aromatic ring A fused to a non-aromatic ring A′,

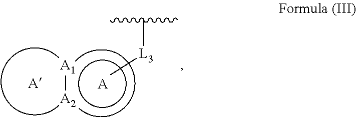

wherein:
L₃ is a single bond;
A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, CF₃, and halo; A′ is a non-aromatic carbon-containing monocyclic ring containing 5, 6, or 7, ring members, wherein at least one ring member is independently selected from N, S, and O;
wherein A′ may be optionally substituted with one R₇, wherein Ry is selected from alkyl, alkoxy, and —(CH₂) o 3-R₈,

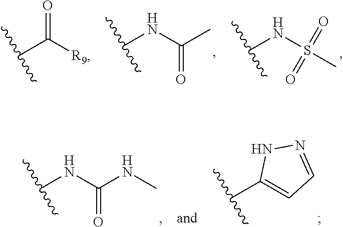

wherein R₈ is selected from cycloalkyl, heterocycloalkyl, wherein R₉ is selected from H, alkyl, OH, NH₂, and alkoxy;
wherein A′ may be optionally substituted with 1 or 2 further substituents R₁₀ and R₁₁, wherein R₁₀ and R₁₁ are independently selected from alkyl, cycloalkyl, alkoxy, OH, oxo, CF₃, and halo, or wherein A′ may be optionally substituted with 1 further substituent R₂₄ selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O.
R₁ can be Formula (III) comprising an aromatic ring A fused to a non-aromatic ring A′,

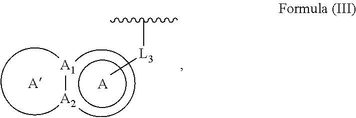

wherein:
L₃ is a single bond;
A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, O(cyclopropyl), CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R₇ is selected from alkyl, alkoxy, and

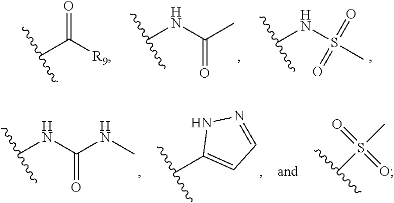

wherein R₈ is selected from cycloalkyl, heterocycloalkyl, wherein R₉ is selected from H, alkyl, OH, N(R₃₁)(R₃₂), and alkoxy;
wherein R₃₁ is H or small alkyl;
wherein R₃₂ is selected from H, small alkyl, and —(CH₂)₀₋₃heteroaryl;
wherein A′ may be optionally substituted with 1, 2, or 3 further substituents R₁₀, R₁₁, and R₁₇, wherein R₁₀, R₁₁, and R₁₇, are independently selected from alkyl, cycloalkyl, alkoxy, OH, oxo, CF₃, and halo, or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O, or wherein A′ may be optionally substituted with 1 or 2 further substituents R₄₃ and R₄₄ independently selected from alkyl, cycloalkyl, alkoxy, OH, oxo, CF₃, and halo, and two different carbon ring members within A′ are connected by a bridging —CH₂-group.
R₁ can be Formula (III) comprising an aromatic ring A fused to a non-aromatic ring A′,

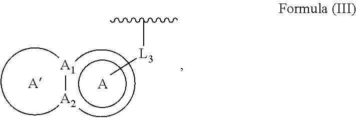

wherein:
L₃ is a single bond;
A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R₇ is selected from alkyl, alkoxy, and —(CH₂)₀₋₃-R₈, wherein R₈ is selected from cycloalkyl, heterocycloalkyl,

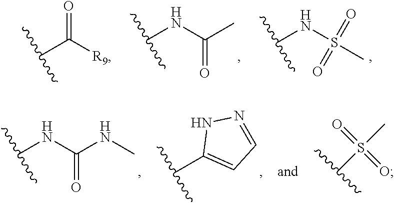

wherein R₉ is selected from H, alkyl, OH, N(R₃₁)(R₃₂), and alkoxy;
wherein R₃₁ is H or small alkyl;
wherein R₃₂ is selected from H, small alkyl, and —(CH₂)₀₋₃heteroaryl;
wherein A′ may be optionally substituted with 1, 2, or 3 further substituents R₁₀, R₁₁, and R₁₇, wherein R₁₀, R₁₁, and R₁₇, are independently selected from alkyl, cycloalkyl, alkoxy, OH, oxo, CF₃, and halo, or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O.
R₁ can be Formula (III) comprising an aromatic ring A fused to a non-aromatic ring A′,

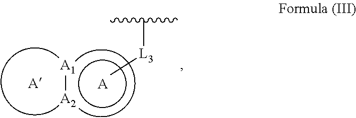

wherein:
L₃ is a single bond;
A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R₇ is selected from alkyl, alkoxy, and —(CH₂)₀₋₃-R₈, wherein R₈ is selected from cycloalkyl, heterocycloalkyl,

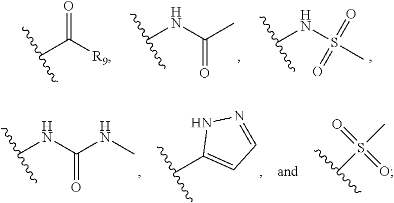

wherein R₉ is selected from H, alkyl, OH, NH₂, and alkoxy;
wherein A′ may be optionally substituted with 1, 2, or 3 further substituents R₁₀, R₁₁, and R₁₇, wherein R₁₀, R₁₁, and R₁₇, are independently selected from alkyl, cycloalkyl, alkoxy, OH, oxo, CF₃, and halo, or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O.
R₁ can be Formula (III) comprising an aromatic ring A fused to a non-aromatic ring A′,

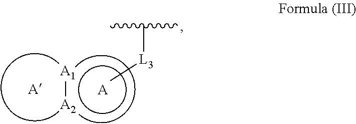

wherein:
L₃ is a single bond;
A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein Ry is selected from alkyl, alkoxy, and

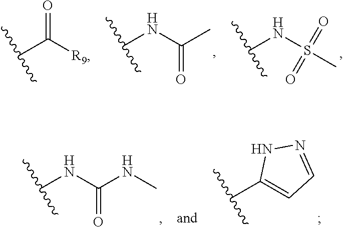

wherein R₈ is selected from cycloalkyl, heterocycloalkyl, wherein R₉ is selected from H, alkyl, OH, NH₂, and alkoxy;
wherein A′ may be optionally substituted with 1 or 2 further substituents R₁₀ and R₁, wherein R₁₀ and R₁₁ are independently selected from alkyl, cycloalkyl, alkoxy, OH, oxo, CF₃, and halo, or wherein A′ may be optionally substituted with 1 further substituent R₂₄ selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O.

Formula (III) can comprise an aromatic ring A fused to a non-aromatic ring A′,

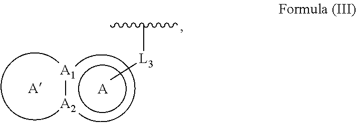

wherein:
L₃ is a single bond;
A₁ and A₂ are both carbon;
A is phenyl or a 6-membered carbon-containing aromatic ring containing one, two or three nitrogen ring members;
wherein A may be optionally substituted with 1 or 2 substituents independently selected from alkyl, cycloalkyl, alkoxy, OH, O(cyclopropyl), CF₃, and halo; A′ is a non-aromatic carbon-containing monocyclic ring containing 5, 6, or 7, ring members, wherein one ring member is N or wherein one ring member is N and a second ring member is O;
wherein A′ may be optionally substituted with one R₇, wherein R₇ is selected from alkyl, alkoxy, and

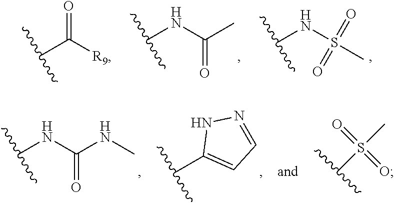

wherein R₈ is selected from cycloalkyl, heterocycloalkyl, wherein R₉ is selected from H, alkyl, OH, N(R₃₁)(R₃₂), and alkoxy;
wherein R₃₁ is H or small alkyl;
wherein R₃₂ is selected from H, small alkyl, and —(CH₂)₀₋₃heteroaryl;
wherein A′ may be optionally substituted with 1, 2, or 3 further substituents R₁₀, R₁₁, and R₁₇, wherein R₁₀, R₁₁, and R₁₇, are independently selected from alkyl, cycloalkyl, alkoxy, OH, oxo, CF₃, and halo, or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O, or wherein A′ may be optionally substituted with 1 or 2 further substituents R₄₃ and R₄₄ independently selected from alkyl, cycloalkyl, alkoxy, OH, oxo, CF₃, and halo, and two different carbon ring members within A′ are connected by a bridging —CH₂-group.

Formula (III) can comprise an aromatic ring A fused to a non-aromatic ring A′,

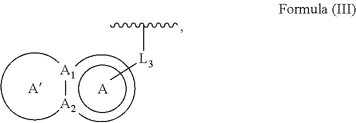

wherein:
L₃ is a single bond;
A₁ and A₂ are both carbon; A is phenyl or a 6-membered carbon-containing aromatic ring containing one, two or three nitrogen ring members;
wherein A may be optionally substituted with 1 or 2 substituents independently selected from alkyl, cycloalkyl, alkoxy, OH, CF₃, and halo;

A′ is a non-aromatic carbon-containing monocyclic ring containing 5, 6, or 7, ring members, wherein at least one ring member is N;
wherein A′ may be optionally substituted with one R₇, wherein R₇ is selected from alkyl, alkoxy, and —(CH₂)₀₋₃-R₈, wherein R₈ is selected from cycloalkyl, heterocycloalkyl,

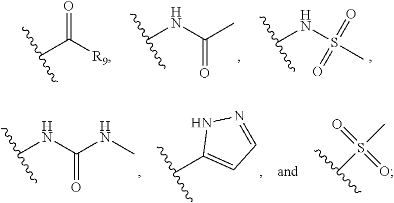

wherein R₉ is selected from H, alkyl, OH, N(R₃₁)(R₃₂), and alkoxy;
wherein R₃₁ is H or small alkyl;
wherein R₃₂ is selected from H, small alkyl, and —(CH₂)₀₋₃heteroaryl;
wherein A′ may be optionally substituted with 1, 2, or 3 further substituents R₁₀, R₁₁, and R₁₇, wherein R₁₀, R₁₁, and R₁₇, are independently selected from alkyl, cycloalkyl, alkoxy, OH, oxo, CF₃, and halo, or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O.

Formula (III) can comprise an aromatic ring A fused to a non-aromatic ring A′,

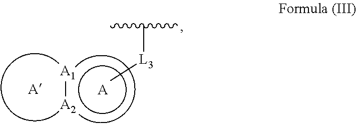

wherein:
L₃ is a single bond;
A₁ and A₂ are both carbon;
A is phenyl or a 6-membered carbon-containing aromatic ring containing one, two or three nitrogen ring members;
wherein A may be optionally substituted with 1 or 2 substituents independently selected from alkyl, cycloalkyl, alkoxy, OH, CF₃, and halo;

A′ is a non-aromatic carbon-containing monocyclic ring containing 5, 6, or 7, ring members, wherein at least one ring member is N;
wherein A′ may be optionally substituted with one R₇, wherein R₇ is selected from alkyl, alkoxy, and —(CH₂)₀₋₃-R₈, wherein R₈ is selected from cycloalkyl, heterocycloalkyl,

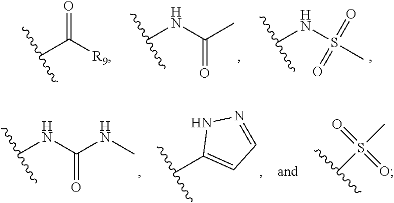

wherein R₉ is selected from H, alkyl, OH, NH₂, and alkoxy;
wherein A′ may be optionally substituted with 1, 2, or 3 further substituents R₁₀, R₁₁, and R₁₇, wherein R₁₀, R₁₁, and R₁₇, are independently selected from alkyl, cycloalkyl, alkoxy, OH, oxo, CF₃, and halo, or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O.

Formula (III) can comprise an aromatic ring A fused to a non-aromatic ring A′,

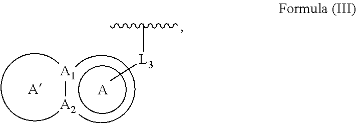

wherein:
L₃ is a single bond;
A₁ and A₂ are both carbon;
A is phenyl or a 6-membered carbon-containing aromatic ring containing one, two or three nitrogen ring members;
wherein A may be optionally substituted with 1 or 2 substituents independently selected from alkyl, cycloalkyl, alkoxy, OH, CF₃, and halo;

A′ is a non-aromatic carbon-containing monocyclic ring containing 5, 6, or 7, ring members, wherein at least one ring member is N;
wherein A′ may be optionally substituted with one R₇, wherein R₇ is selected from alkyl, alkoxy, and —(CH₂)₀₋₃-R₈, wherein R₈ is selected from cycloalkyl, heterocycloalkyl,

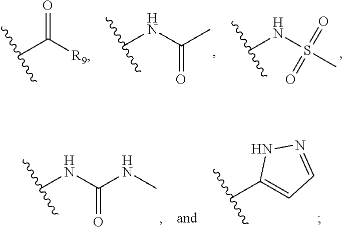

wherein R₉ is selected from H, alkyl, OH, NH₂, and alkoxy;
wherein A′ may be optionally substituted with 1 or 2 further substituents R₁₀ and R₁, wherein R₁₀ and R₁₁ are independently selected from alkyl, cycloalkyl, alkoxy, OH, oxo, CF₃, and halo, or wherein A′ may be optionally substituted with 1 further substituent R₂₄ selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O.
R₁ can be Formula (III) comprising an aromatic ring A fused to a non-aromatic ring A′,

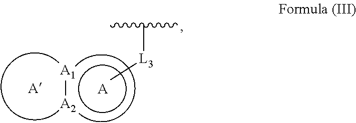

wherein:
L₃ is a single bond;
A₁ and A₂ are both carbon;
A is phenyl or a 6-membered carbon-containing aromatic ring containing one, two or three nitrogen ring members;
wherein A may be optionally substituted with 1 or 2 substituents independently selected from alkyl, cycloalkyl, alkoxy, OH, O(cyclopropyl), CF₃, and halo; A′ is a non-aromatic carbon-containing monocyclic ring containing 5, 6, or 7, ring members, wherein one ring member is N or wherein one ring member is N and a second ring member is O;
wherein A′ may be optionally substituted with one R₇, wherein R₇ is selected from alkyl, alkoxy, and

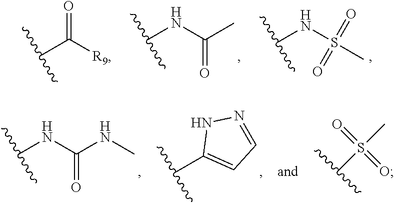

wherein R₈ is selected from cycloalkyl, heterocycloalkyl, wherein R₉ is selected from H, alkyl, OH, N(R₃₁)(R₃₂), and alkoxy;
wherein R₃₁ is H or small alkyl;
wherein R₃₂ is selected from H, small alkyl, and —(CH₂)₀₋₃heteroaryl;
wherein A′ may be optionally substituted with 1, 2, or 3 further substituents R₁₀, R₁₁, and R₁₇, wherein R₁₀, R₁₁, and R₁₇, are independently selected from alkyl, cycloalkyl, alkoxy, OH, oxo, CF₃, and halo, or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O, or wherein A′ may be optionally substituted with 1 or 2 further substituents R₄₃ and R₄₄ independently selected from alkyl, cycloalkyl, alkoxy, OH, oxo, CF₃, and halo, and two different carbon ring members within A′ are connected by a bridging —CH₂-group.
R₁ can be Formula (III) comprising an aromatic ring A fused to a non-aromatic ring A′,

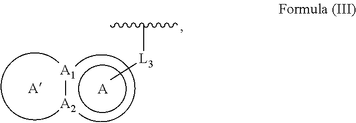

wherein:
L₃ is a single bond;
A₁ and A₂ are both carbon; A is phenyl or a 6-membered carbon-containing aromatic ring containing one, two or three nitrogen ring members;
wherein A may be optionally substituted with 1 or 2 substituents independently selected from alkyl, cycloalkyl, alkoxy, OH, CF₃, and halo;

A′ is a non-aromatic carbon-containing monocyclic ring containing 5, 6, or 7, ring members, wherein at least one ring member is N;
wherein A′ may be optionally substituted with one R₇, wherein R₇ is selected from alkyl, alkoxy, and —(CH₂)₀₋₃-R₈, wherein R₈ is selected from cycloalkyl, heterocycloalkyl,

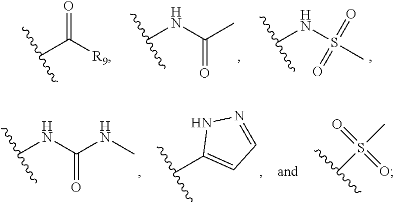

wherein R₉ is selected from H, alkyl, OH, N(R₃₁)(R₃₂), and alkoxy;
wherein R₃₁ is H or small alkyl;
wherein R₃₂ is selected from H, small alkyl, and —(CH₂)₀₋₃heteroaryl;
wherein A′ may be optionally substituted with 1, 2, or 3 further substituents R₁₀, R₁₁, and R₁₇, wherein R₁₀, R₁₁, and R₁₇, are independently selected from alkyl, cycloalkyl, alkoxy, OH, oxo, CF₃, and halo, or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O.
R₁ can be Formula (III) comprising an aromatic ring A fused to a non-aromatic ring A′,

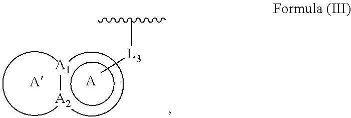

wherein:
L₃ is a single bond;
A₁ and A₂ are both carbon;
A is phenyl or a 6-membered carbon-containing aromatic ring containing one, two or three nitrogen ring members;
wherein A may be optionally substituted with 1 or 2 substituents independently selected from alkyl, cycloalkyl, alkoxy, OH, CF₃, and halo;

A′ is a non-aromatic carbon-containing monocyclic ring containing 5, 6, or 7, ring members, wherein at least one ring member is N;
wherein A′ may be optionally substituted with one R₇, wherein R₇ is selected from alkyl, alkoxy, and —(CH₂)₀₋₃—R₈, wherein R₈ is selected from cycloalkyl, heterocycloalkyl,

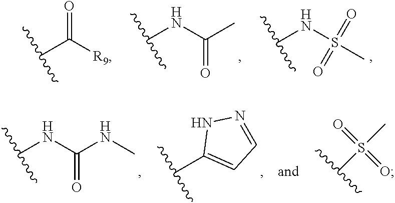

wherein R₉ is selected from H, alkyl, OH, NH₂, and alkoxy;
wherein A′ may be optionally substituted with 1, 2, or 3 further substituents R₁₀, R₁₁, and R₁₇, wherein R₁₀, R₁₁, and R₁₇, are independently selected from alkyl, cycloalkyl, alkoxy, OH, oxo, CF₃, and halo, or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O.
R₁ can be Formula (III) comprising an aromatic ring A fused to a non-aromatic ring A′,

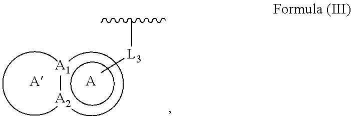

wherein:
L₃ is a single bond;
A₁ and A₂ are both carbon; A is phenyl or a 6-membered carbon-containing aromatic ring containing one, two or three nitrogen ring members;
wherein A may be optionally substituted with 1 or 2 substituents independently selected from alkyl, cycloalkyl, alkoxy, OH, CF₃, and halo;

A′ is a non-aromatic carbon-containing monocyclic ring containing 5, 6, or 7, ring members, wherein at least one ring member is N;
wherein A′ may be optionally substituted with one R₇, wherein R, is selected from alkyl, alkoxy, and —(CH₂) o 3-R₈, wherein R₈ is selected from cycloalkyl, heterocycloalkyl,

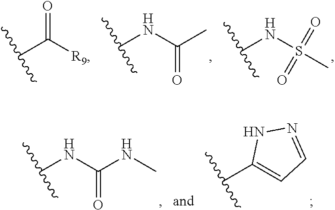

wherein R₈ is selected from H, alkyl, OH, NH₂, and alkoxy;
wherein A′ may be optionally substituted with 1 or 2 further substituents R₁₀ and R₁₁, wherein R₁₀ and R₁₁ are independently selected from alkyl, cycloalkyl, alkoxy, OH, oxo, CF₃, and halo, or wherein A′ may be optionally substituted with 1 further substituent R₂₄ selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O.

Formula (III) can comprise an aromatic ring A fused to a non-aromatic ring A′,

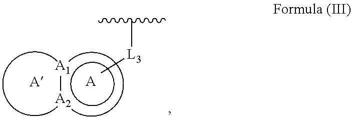

wherein:
L₃ is a single bond;
A₁ and A₂ are both carbon;
A is phenyl or pyridyl;

A′ can be a non-aromatic carbon-containing monocyclic ring containing 6 or 7 ring members, wherein one ring member is N or wherein one ring member is N and a second ring member is O; wherein A′ may be optionally substituted with one R₇, wherein R, is selected from alkyl, alkoxy, and

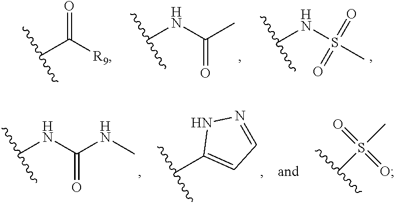

wherein R₈ is selected from cycloalkyl, heterocycloalkyl, wherein R₉ is selected from H, alkyl, OH, N(R₃₁)(R₃₂), and alkoxy;
wherein R₃₁ is H or small alkyl;
wherein R₃₂ is selected from H, small alkyl, and —(CH₂)₀₋₃heteroaryl;
wherein A′ may be optionally substituted with 1, 2, or 3 further substituents R₁₀, R₁₁, and R₁₇, wherein R₁₀, R₁₁, and R₁₇, are independently selected from alkyl, cycloalkyl, alkoxy, OH, oxo, CF₃, and halo, or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O, or wherein A′ may be optionally substituted with 1 or 2 further substituents R₄₃ and R₄₄ independently selected from alkyl, cycloalkyl, alkoxy, OH, oxo, CF₃, and halo, and two different carbon ring members within A′ are connected by a bridging —CH₂-group.

Formula (III) can comprise an aromatic ring A fused to a non-aromatic ring A′,

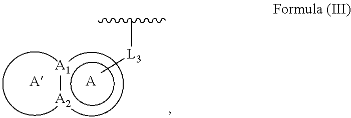

wherein:
L₃ is a single bond;
A₁ and Az are both carbon;
A is phenyl or pyridyl;

A′ can be a non-aromatic carbon-containing monocyclic ring containing 6 ring members, wherein at least one ring member is N;
wherein A′ may be optionally substituted with one R₇, wherein R₇ is selected from alkyl, alkoxy, and

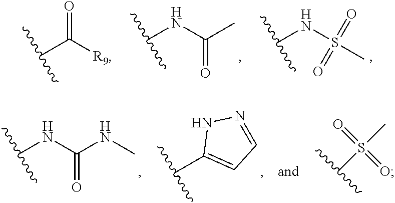

wherein R₈ is selected from cycloalkyl, heterocycloalkyl, wherein R₉ is selected from H, alkyl, OH, N(R₃₁)(R₃₂), and alkoxy;
wherein R₃₁ is H or small alkyl;
wherein R₃₂ is selected from H, small alkyl, and —(CH₂)₀₋₃heteroaryl;
wherein A′ may be optionally substituted with 1, 2, or 3 further substituents R₁₀, R₁₁, and R₁₇, wherein R₁₀, R₁₁, and R₁₇, are independently selected from alkyl, cycloalkyl, alkoxy, OH, oxo, CF₃, and halo, or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O.

Formula (III) can comprise an aromatic ring A fused to a non-aromatic ring A′,

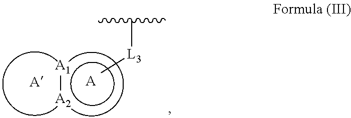

wherein:
L₃ is a single bond;
A₁ and A₂ are both carbon;
A is phenyl or pyridyl;

A′ can be a non-aromatic carbon-containing monocyclic ring containing 6 ring members, wherein at least one ring member is N;
wherein A′ may be optionally substituted with one R₇, wherein R₇ is selected from alkyl, alkoxy, and —(CH₂) o 3-R₈,

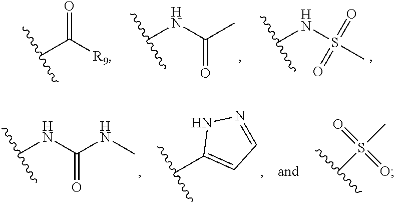

wherein R₈ is selected from cycloalkyl, heterocycloalkyl, wherein R₉ is selected from H, alkyl, OH, NH₂, and alkoxy;
wherein A′ may be optionally substituted with 1, 2, or 3 further substituents R₁₀, R₁₁, and R₁₇, wherein R₁₀, R₁₁, and R₁₇, are independently selected from alkyl, cycloalkyl, alkoxy, OH, oxo, CF₃, and halo, or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O.

Formula (III) can comprise an aromatic ring A fused to a non-aromatic ring A′,

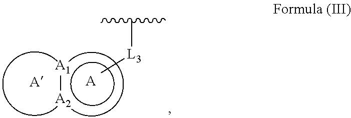

wherein:
L₃ is a single bond;
A₁ and A₂ are both carbon;
A is phenyl or pyridyl;

A′ can be a non-aromatic carbon-containing monocyclic ring containing 6 ring members, wherein at least one ring member is N;
wherein A′ may be optionally substituted with one R₇, wherein R₇ is selected from alkyl, alkoxy, and —(CH₂)₀₋₃—R₈,
wherein R₈ is selected from cycloalkyl, heterocycloalkyl,

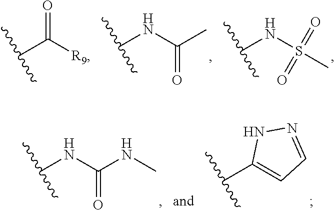

wherein R₉ is selected from H, alkyl, OH, NH₂, and alkoxy;
wherein A′ may be optionally substituted with 1 or 2 further substituents R₁₀ and R₁₁, wherein R₁₀ and R₁₁ are independently selected from alkyl, cycloalkyl, alkoxy, OH, oxo, CF₃, and halo, or wherein A′ may be optionally substituted with 1 further substituent R₂₄ selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O; and wherein R₇, when present, is bonded to A′ via a N ring member.
R₁ can be Formula (III) comprising an aromatic ring A fused to a non-aromatic ring A′,

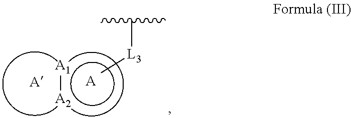

wherein:
L₃ is a single bond;
A₁ and A₂ are both carbon;
A is phenyl or pyridyl;

A′ can be a non-aromatic carbon-containing monocyclic ring containing 6 or 7 ring members, wherein one ring member is N or wherein one ring member is N and a second ring member is O; wherein A′ may be optionally substituted with one R₇, wherein R₇ is selected from alkyl, alkoxy, and —(CH₂)₀₋₃-R₈, wherein R₈ is selected from cycloalkyl, heterocycloalkyl,

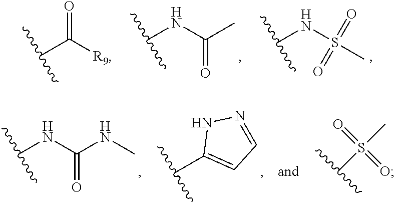

wherein R₉ is selected from H, alkyl, OH, N(R₃₁)(R₃₂), and alkoxy;
wherein R₃₁ is H or small alkyl;
wherein R₃₂ is selected from H, small alkyl, and —(CH₂)₀₋₃heteroaryl;
wherein A′ may be optionally substituted with 1, 2, or 3 further substituents R₁₀, R₁₁, and R₁₇, wherein R₁₀, R₁₁, and R₁₇, are independently selected from alkyl, cycloalkyl, alkoxy, OH, oxo, CF₃, and halo, or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O, or wherein A′ may be optionally substituted with 1 or 2 further substituents R₄₃ and R₄₄ independently selected from alkyl, cycloalkyl, alkoxy, OH, oxo, CF₃, and halo, and two different carbon ring members within A′ are connected by a bridging —CH₂-group.
R₁ can be Formula (III) comprising an aromatic ring A fused to a non-aromatic ring A′,

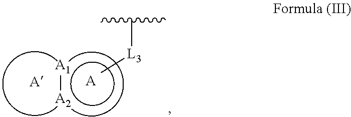

wherein:
L₃ is a single bond;
A₁ and A₂ are both carbon;
A is phenyl or pyridyl;

A′ can be a non-aromatic carbon-containing monocyclic ring containing 6 ring members, wherein at least one ring member is N;
wherein A′ may be optionally substituted with one R₇, wherein R₇ is selected from alkyl, alkoxy, and —(CH₂)₀₋₃-R₈,
wherein R₈ is selected from cycloalkyl, heterocycloalkyl,

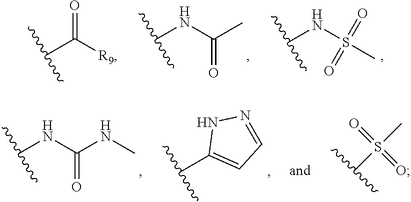

wherein R₉ is selected from H, alkyl, OH, N(R₃₁)(R₃₂), and alkoxy;
wherein R₃₁ is H or small alkyl;
wherein R₃₂ is selected from H, small alkyl, and —(CH₂)₀₋₃heteroaryl;
wherein A′ may be optionally substituted with 1, 2, or 3 further substituents R₁₀, R₁₁, and R₁₇, wherein R₁₀, R₁₁, and R₁₇, are independently selected from alkyl, cycloalkyl, alkoxy, OH, oxo, CF₃, and halo, or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O.
R₁ can be Formula (III) comprising an aromatic ring A fused to a non-aromatic ring A′,

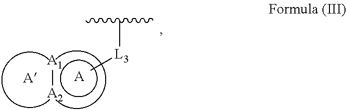

wherein:
L₃ is a single bond;
A₁ and A₂ are both carbon;
A is phenyl or pyridyl;

A′ can be a non-aromatic carbon-containing monocyclic ring containing 6 ring members, wherein at least one ring member is N;
wherein A′ may be optionally substituted with one R₇, wherein R, is selected from alkyl, alkoxy, and —(CH₂)₀₋₃—R₈,

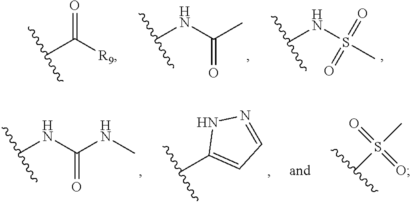

wherein R₈ is selected from cycloalkyl, heterocycloalkyl, wherein R₉ is selected from H, alkyl, OH, NH₂, and alkoxy;
wherein A′ may be optionally substituted with 1, 2, or 3 further substituents R₁₀, R₁₁, and R₁₇, wherein R₁₀, R₁₁, and R₁₇, are independently selected from alkyl, cycloalkyl, alkoxy, OH, oxo, CF₃, and halo, or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O.
R₁ can be Formula (III) comprising an aromatic ring A fused to a non-aromatic ring A′,

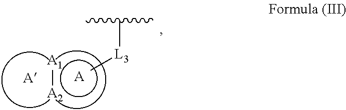

wherein:
L₃ is a single bond;
A₁ and A₂ are both carbon;
A is phenyl or pyridyl;

A′ can be a non-aromatic carbon-containing monocyclic ring containing 6 ring members, wherein at least one ring member is N;
wherein A′ may be optionally substituted with one Ry, wherein Ry is selected from alkyl, alkoxy, and —(CH₂)₀₋₃—R₈,
wherein R₈ is selected from cycloalkyl, heterocycloalkyl,

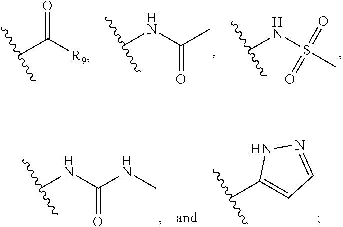

wherein R₈ is selected from H, alkyl, OH, NH₂, and alkoxy;
wherein A′ may be optionally substituted with 1 or 2 further substituents R₁₀ and R₁₁, wherein R₁₀ and R₁₁ are independently selected from alkyl, cycloalkyl, alkoxy, OH, oxo, CF₃, and halo, or wherein A′ may be optionally substituted with 1 further substituent R₂₄ selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O; and wherein R₇, when present, is bonded to A′ via a N ring member.

Formula (III) can be represented by Formula (IIIa), Formula (IIIb), Formula (IIIc), and Formula (IIId),

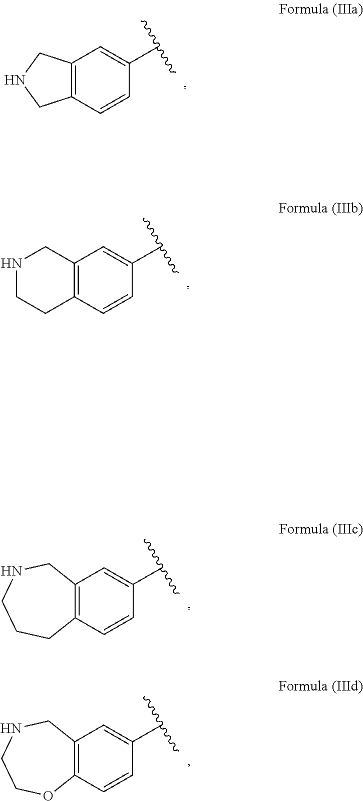

wherein:
A is a phenyl or pyridyl ring and A may be optionally substituted with 1 or 2 substituents independently selected from alkyl, cyclopropyl, alkoxy, OH, CF₃, and halo;

A′ is a fused pyrrolidine, piperidine, azepane, or oxazepane ring;
R₇, when present, is bonded to A′ via the N ring member of Formula (IIIa), Formula (IIIb), Formula (IIIc), or Formula (IIId), replacing H;

A′ may be optionally substituted with 1, 2, or 3 further substituents R₁₀, R₁₁, and R₁₇, wherein R₁₀, R₁₁, and
R₁₇, are independently selected from small alkyl, cyclopropyl, small alkoxy, OH, oxo, CF₃, and halo, or A′ may be optionally substituted with 1 or 2 further substituents R₂₄ and R₂₇ independently selected from small alkyl, cyclopropyl, small alkoxy, OH, oxo, CF₃, and halo, and 2 further substituents R₂₅ and R₂₆, wherein R₂₅ and R₂₆ together with a carbon atom to which they are both attached form a 3-, 4-, or 5-membered non-aromatic monocyclic carbon-containing ring optionally wherein one or two ring members are selected from N, S, and O, or wherein A′ may be optionally substituted with 1 or 2 further substituents R₄₃ and R₄₄ independently selected from alkyl, cycloalkyl, alkoxy, OH, oxo, CF₃, and halo, and two different carbon ring members within A′ are connected by a bridging —CH₂-group.

Formula (III) can be represented by Formula (IIIa), Formula (IIIb), and Formula (IIIc),

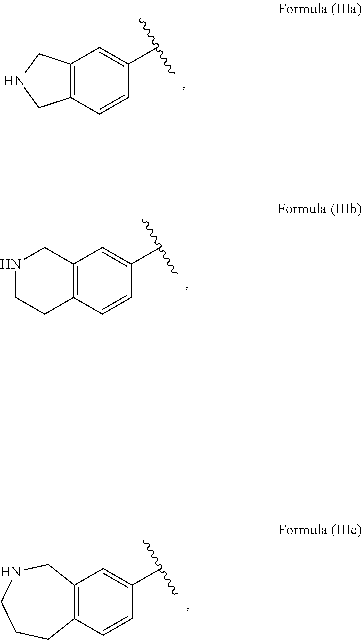

wherein:
A is a phenyl ring and A may be optionally substituted with 1 or 2 substituents independently selected from alkyl, cyclopropyl, alkoxy, OH, CF₃, and halo;

A′ is a fused pyrrolidine, piperidine or azepane ring; R₇, when present, is bonded to A′via the N ring member of Formula (IIIa), Formula (IIIb), or Formula (IIIc), replacing H;

A′ may be optionally substituted with 1, 2, or 3 further substituents R₁₀, R₁₁, and R₁₇, wherein R₁₀, R₁₁, and R₁₇, are independently selected from small alkyl, cyclopropyl, small alkoxy, OH, oxo, CF₃, and halo, or A′ may be optionally substituted with 1 or 2 further substituents R₂₄ and R₂₇ independently selected from small alkyl, cyclopropyl, small alkoxy, OH, oxo, CF₃, and halo, and 2 further substituents R₂₅ and R₂₆, wherein R₂₅ and R₂₆ together with a carbon atom to which they are both attached form a 3-, 4-, or 5-membered non-aromatic monocyclic carbon-containing ring optionally wherein one or two ring members are selected from N, S, and O.

Formula (III) can be represented by Formula (IIIa), Formula (IIIb), and Formula (IIIc),

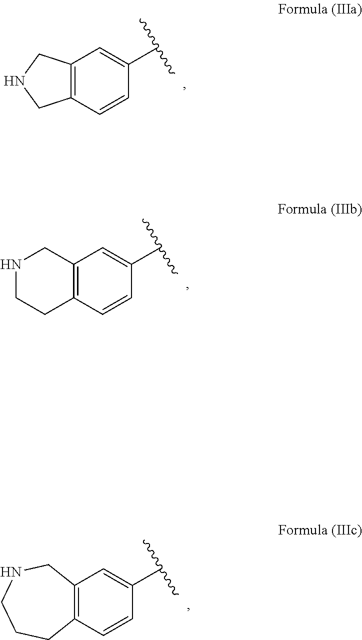

wherein:
A is a phenyl ring and A may be optionally substituted with 1 or 2 substituents independently selected from alkyl, cyclopropyl, alkoxy, OH, CF₃, and halo;

A′ is a fused pyrrolidine, piperidine or azepane ring; R₇, when present, is bonded to A′ via the N ring member of Formula (IIIa), Formula (IIIb), or Formula (IIIc), replacing H;

A′ may be optionally substituted with 1 or 2 further substituents R₁₀ and R₁₁, wherein R₁₀ and R₁₁ are independently selected from small alkyl, cyclopropyl, small alkoxy, OH, oxo, CF₃, and halo, or A′ may be optionally substituted with 1 further substituents R₂₄ selected from small alkyl, cyclopropyl, small alkoxy, OH, oxo, CF₃, and halo, and 2 further substituents R₂₅ and R₂₆, wherein R₂₅ and R₂₆ together with a carbon atom to which they are both attached form a 3-, 4-, or 5-membered non-aromatic monocyclic carbon-containing ring optionally wherein one or two ring members are selected from N, S, and O.

Formula (III) can be represented by Formula (IIIb).
R₁ can be Formula (IIIa), Formula (IIIb), and Formula (IIIc),

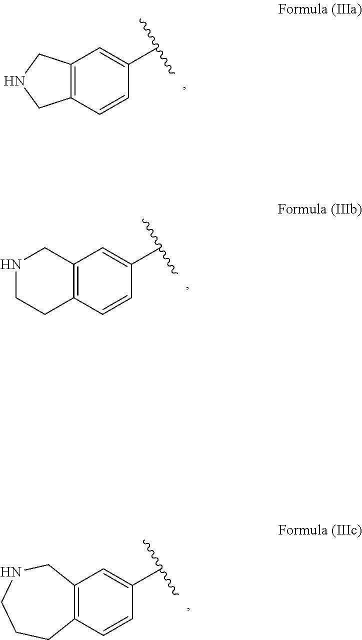

wherein:
A is a phenyl ring and A may be optionally substituted with 1 or 2 substituents independently selected from alkyl, cyclopropyl, alkoxy, OH, CF₃, and halo; A′ is a fused pyrrolidine, piperidine or azepane ring; R₇, when present, is bonded to A′ via the N ring member of Formula (IIIa), Formula (IIIb), or Formula (IIIc), replacing H;

A′ may be optionally substituted with 1, 2, or 3 further substituents R₁₀, R₁₁, and R₁₇, wherein R₁₀, R₁₁, and R₁₇, are independently selected from alkyl, cycloalkyl, alkoxy, OH, oxo, CF₃, and halo, or A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O.
R₁ can be Formula (IIIa), Formula (IIIb), Formula (IIIc), and Formula (IIId),

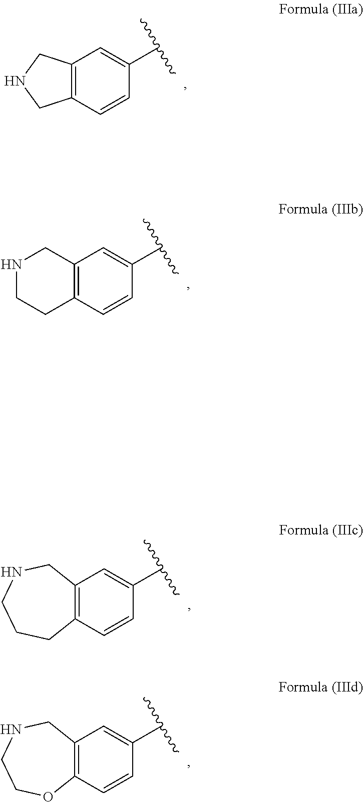

wherein:
A is a phenyl or pyridyl ring and A may be optionally substituted with 1 or 2 substituents independently selected from alkyl, cyclopropyl, alkoxy, OH, CF₃, and halo;

A′ is a fused pyrrolidine, piperidine, azepane, or oxazepane ring;
R₇, when present, is bonded to A′ via the N ring member of Formula (IIIa), Formula (IIIb), Formula (IIIc), or Formula (IIId), replacing H;

A′ may be optionally substituted with 1, 2, or 3 further substituents R₁₀, R₁₁, and R₁₇, wherein R₁₀, R₁₁, and R₁₇, are independently selected from small alkyl, cyclopropyl, small alkoxy, OH, oxo, CF₃, and halo, or A′ may be optionally substituted with 1 or 2 further substituents R₂₄ and R₂₇ independently selected from small alkyl, cyclopropyl, small alkoxy, OH, oxo, CF₃, and halo, and 2 further substituents R₂₅ and R₂₆, wherein R₂₅ and R₂₆ together with a carbon atom to which they are both attached form a 3-, 4-, or 5-membered non-aromatic monocyclic carbon-containing ring optionally wherein one or two ring members are selected from N, S, and O, or wherein A′ may be optionally substituted with 1 or 2 further substituents R₄₃ and R₄₄ independently selected from alkyl, cycloalkyl, alkoxy, OH, oxo, CF₃, and halo, and two different carbon ring members within A′ are connected by a bridging —CH₂-group.
R₁ can be Formula (IIIa), Formula (IIIb), and Formula (IIIc),

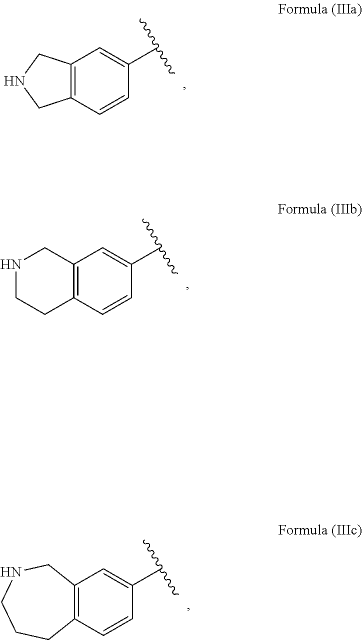

wherein:
A is a phenyl ring and A may be optionally substituted with 1 or 2 substituents independently selected from alkyl, cyclopropyl, alkoxy, OH, CF₃, and halo;

A′ is a fused pyrrolidine, piperidine or azepane ring;
R₇, when present, is bonded to A′ via the N ring member of Formula (IIIa), Formula (IIIb), or Formula (IIIc), replacing H;

A′ may be optionally substituted with 1 or 2 further substituents R₁₀ and R₁₁, wherein R₁₀ and R₁₁ are independently selected from small alkyl, cyclopropyl, small alkoxy, OH, oxo, CF₃, and halo, or A′ may be optionally substituted with 1 further substituents R₂₄ selected from small alkyl, cyclopropyl, small alkoxy, OH, oxo, CF₃, and halo, and 2 further substituents R₂₅ and R₂₆, wherein R₂₅ and R₂₆ together with a carbon atom to which they are both attached form a 3-, 4-, or 5-membered non-aromatic monocyclic carbon-containing ring optionally wherein one or two ring members are selected from N, S, and O.

When Formula (III) is Formula (IIIa), Formula (IIIb), Formula (IIIc), and Formula (IIId), and A′ is oxo

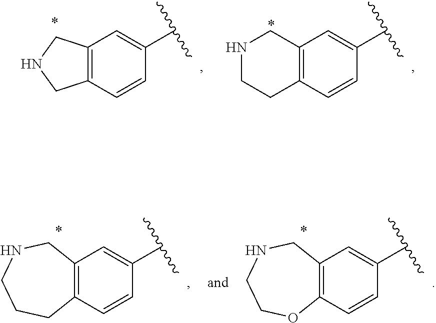 [SMILES: *c1ccc2c(c1)CNC2 ; *c1ccc2c(c1)CNCC2 ; *c1ccc2c(c1)CNCCC2 ; *c1ccc2c(c1)CNCCO2]

substituted, the oxo group may be at the position labelled with a (*):

When Formula (III) is Formula (IIIa), Formula (IIIb), and Formula (IIIc), and A′ is oxo-substituted, the oxo

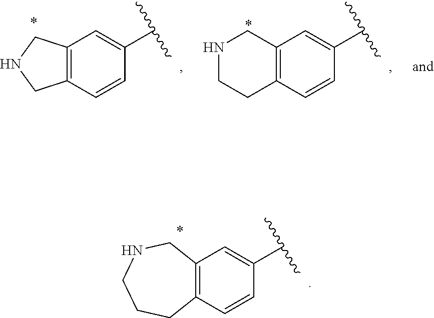 [SMILES: *c1ccc2c(c1)CNC2 ; *c1ccc2c(c1)CNCC2 ; *c1ccc2c(c1)CNCCC2]

group may be at the position labelled with a (*):
R₁ can be Formula (IIIb), Formula (IIIc), or Formula (IIId).
R₁ can be Formula (IIIb) or Formula (IIIc).
R₁ can be Formula (IIIb) or Formula (IIId).
R₁ can be Formula (IIIb).

Formula (III) can be represented by Formula (Ille),

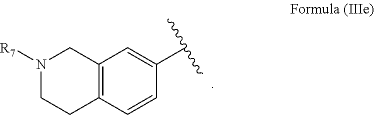

R₁ can be Formula (Ille).
R₁ can be selected from:

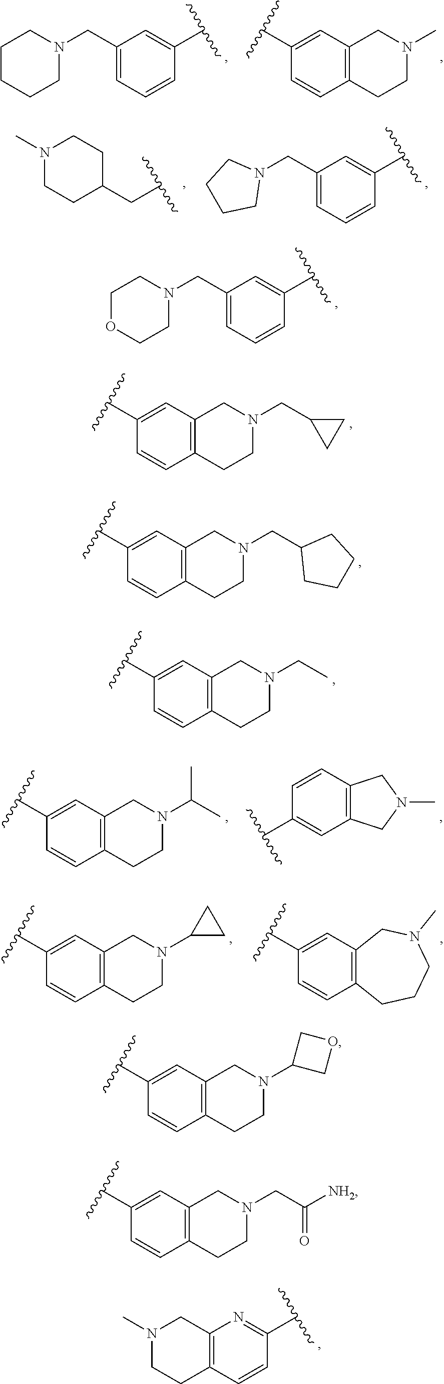

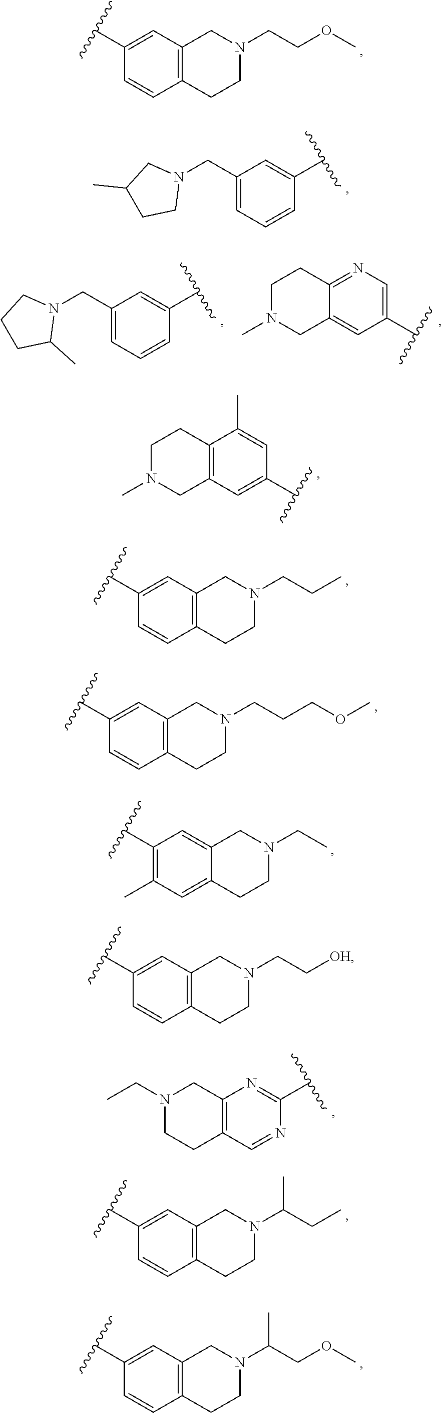

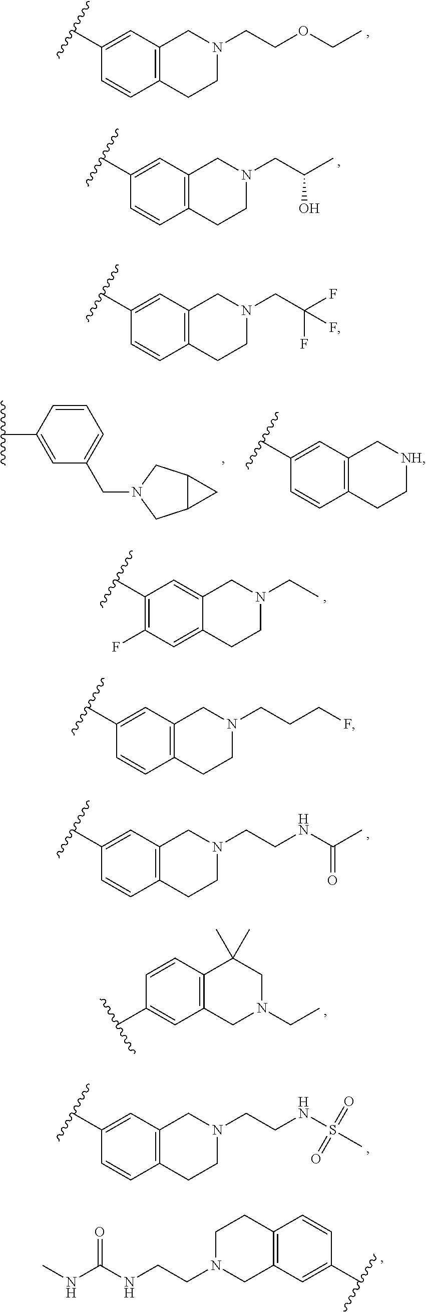

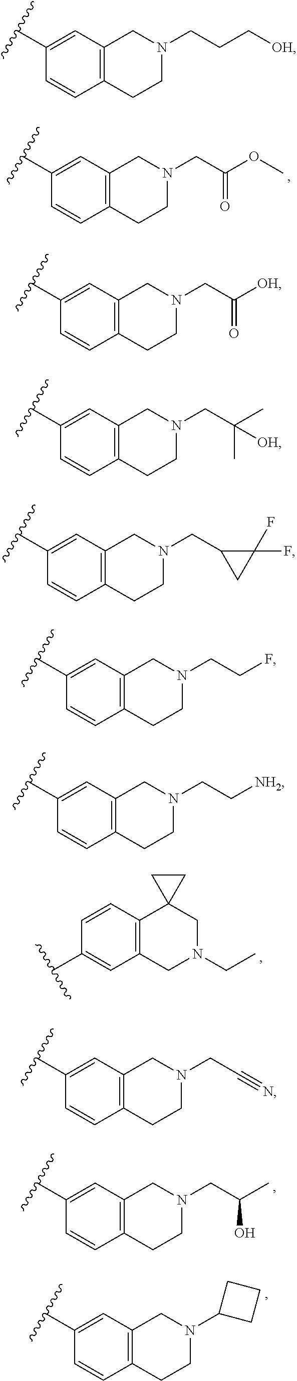

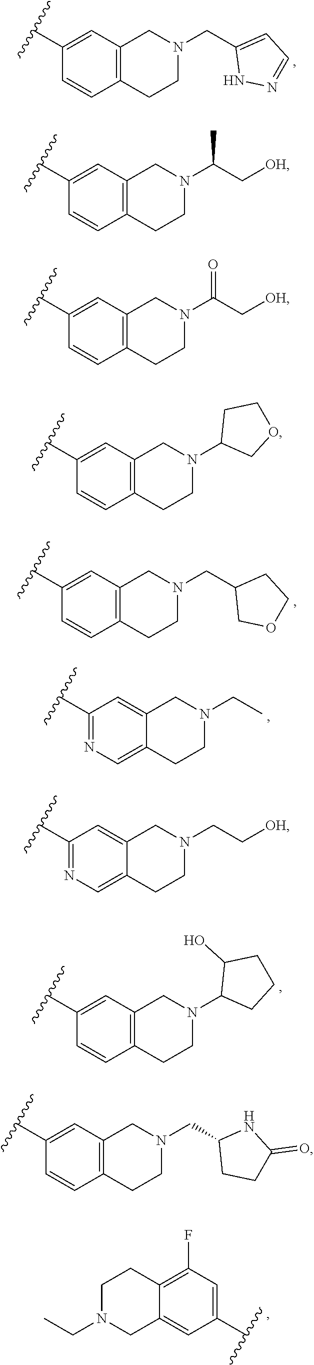

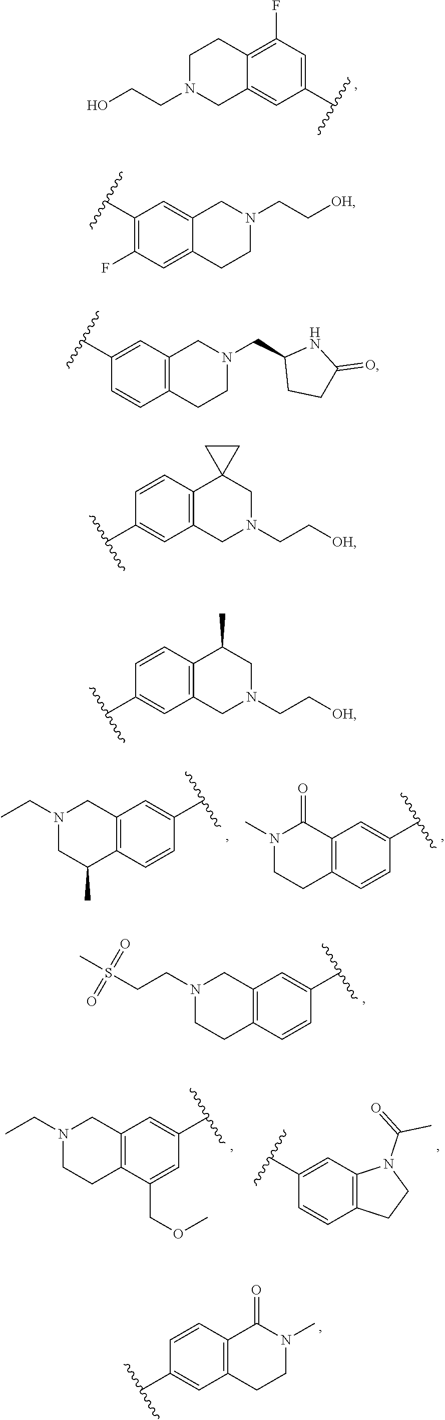

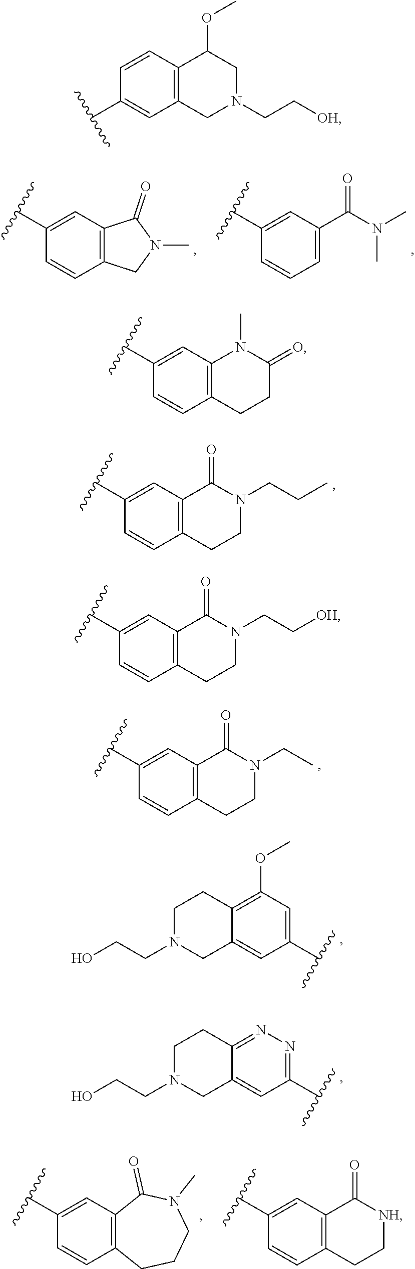

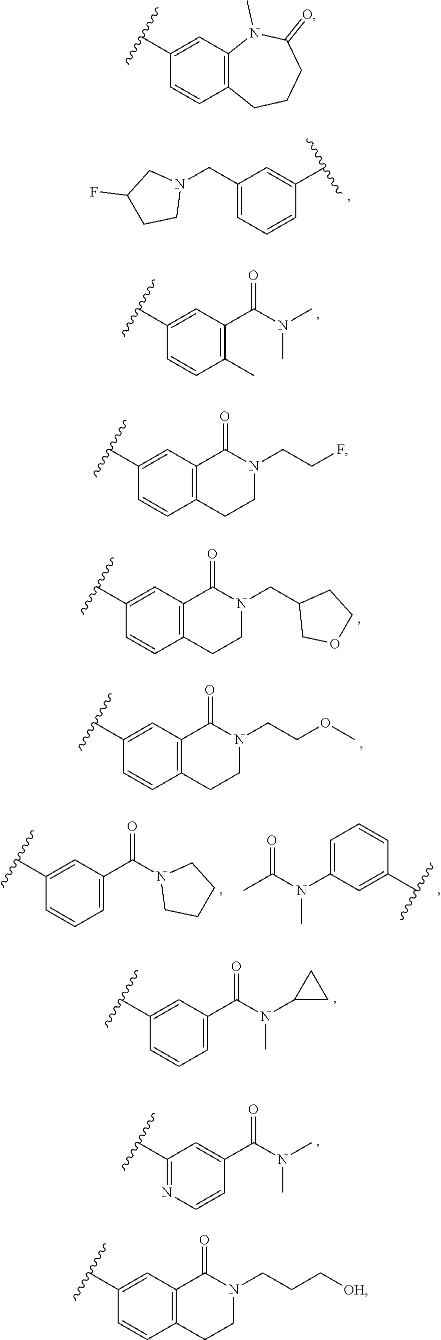

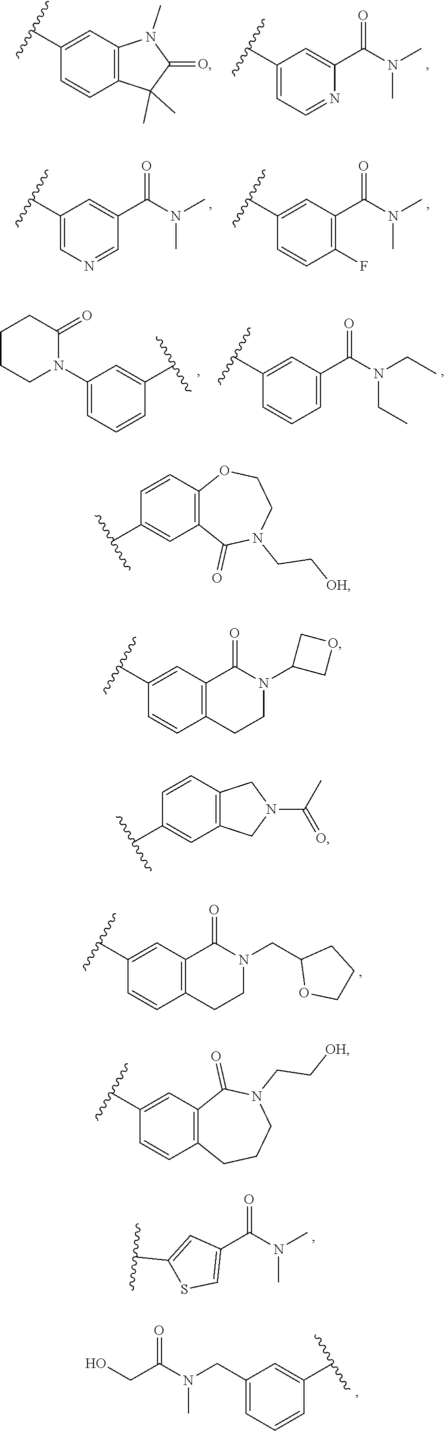

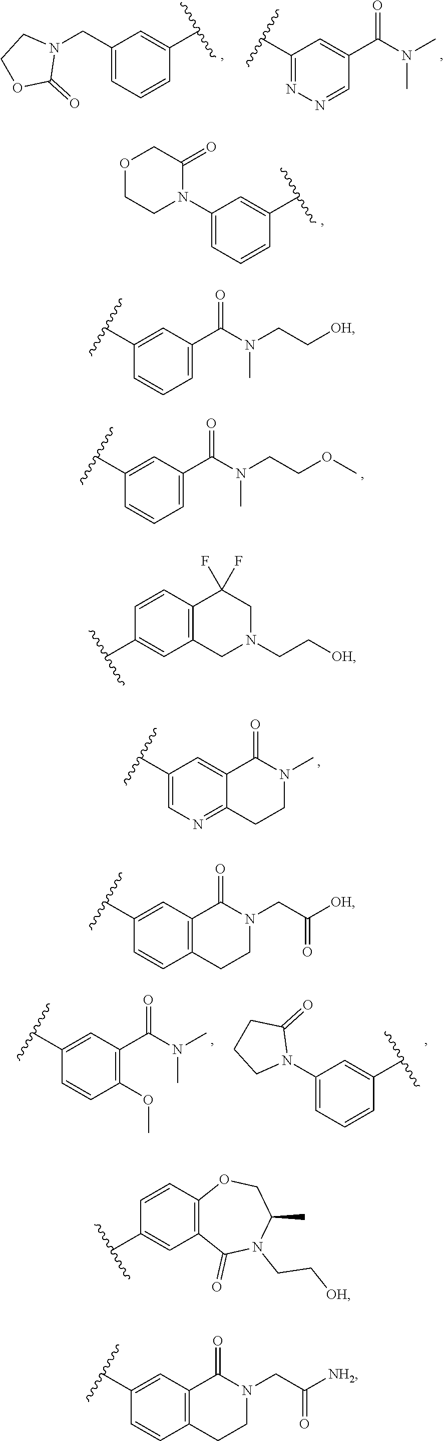

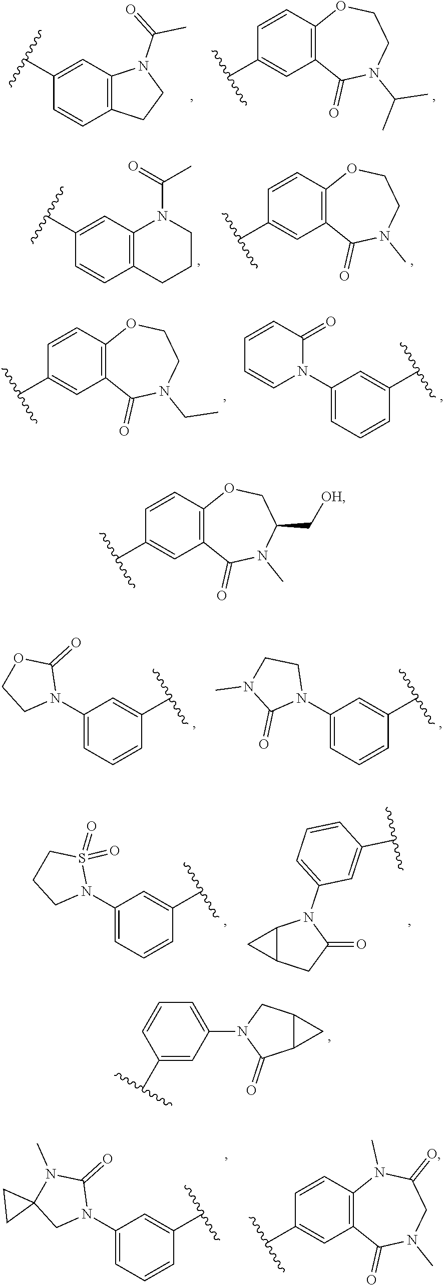

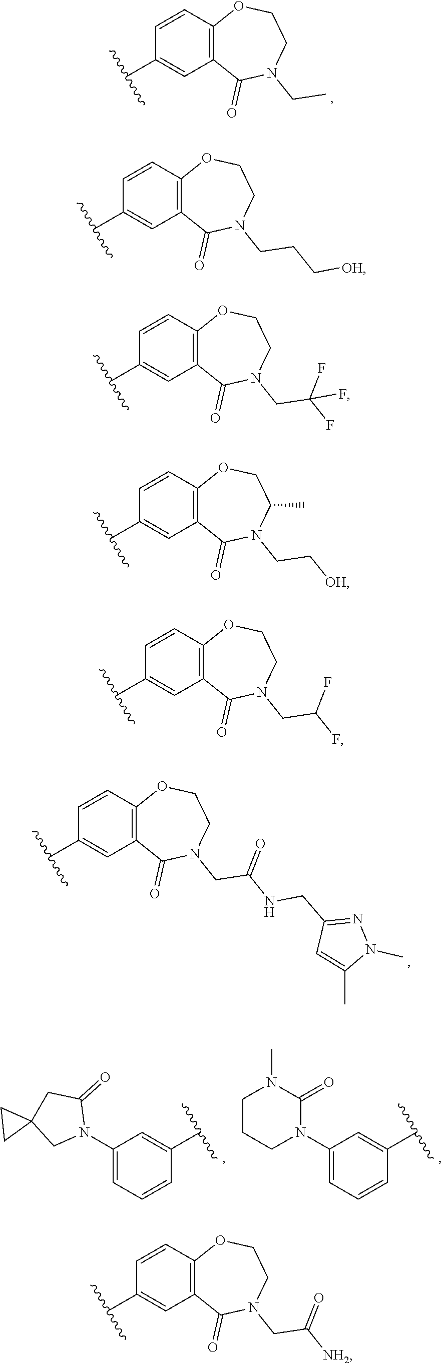

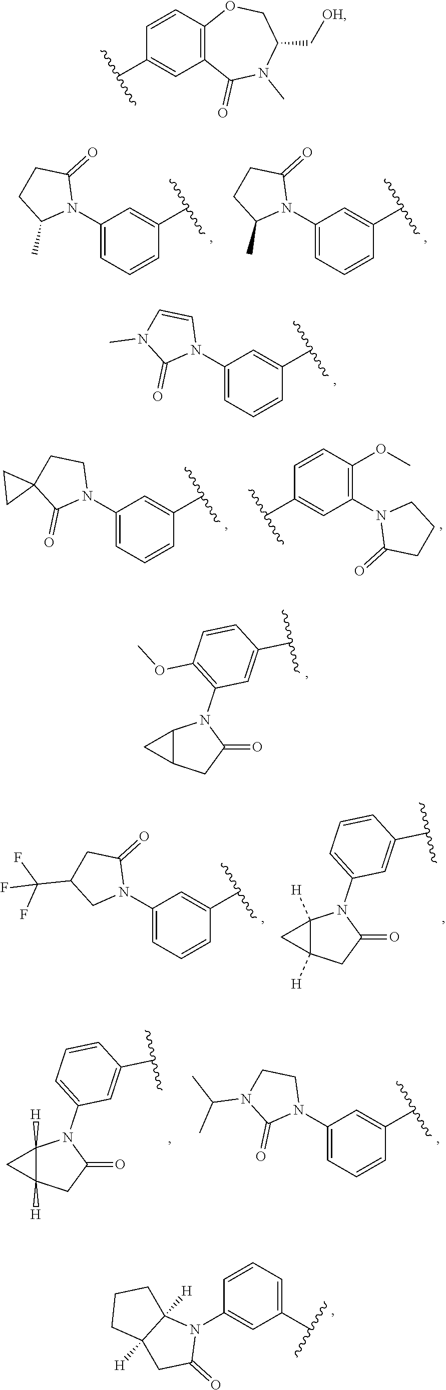

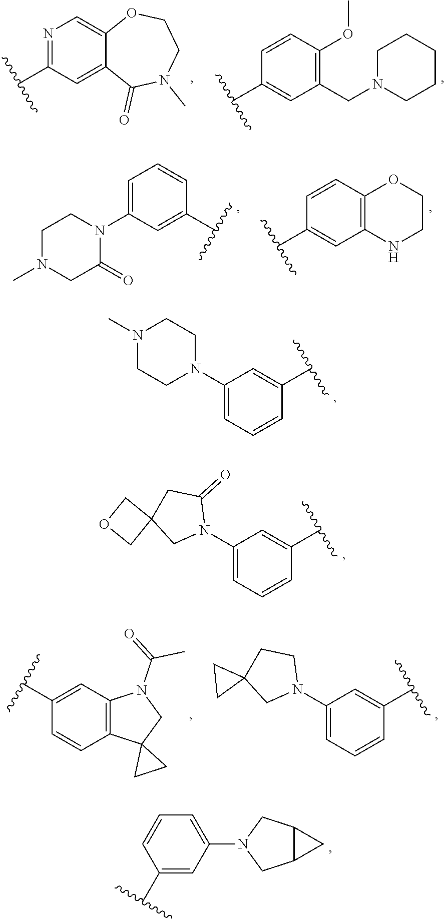

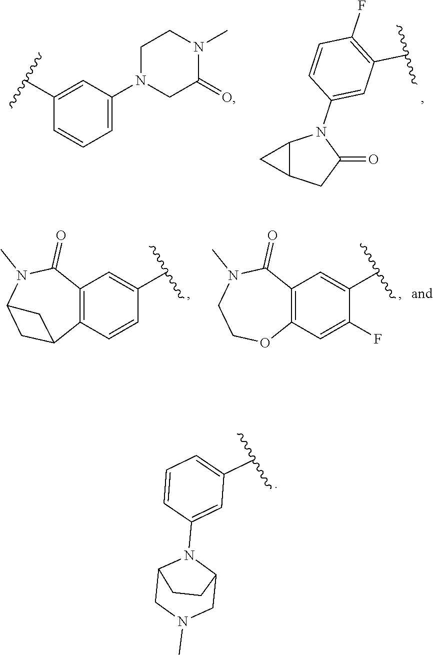 [SMILES: *c1cccc(N2CCN(C)C(=O)C2)c1 ; *c1cc(N2C(=O)CC3CC32)ccc1F ; *c1ccc2c(c1)C(=O)N(C)C1CC2C1 ; *c1cc2c(cc1F)OCCN(C)C2=O ; *c1cccc(N2C3CCC2CN(C)C3)c1]

R₁ can be selected from:

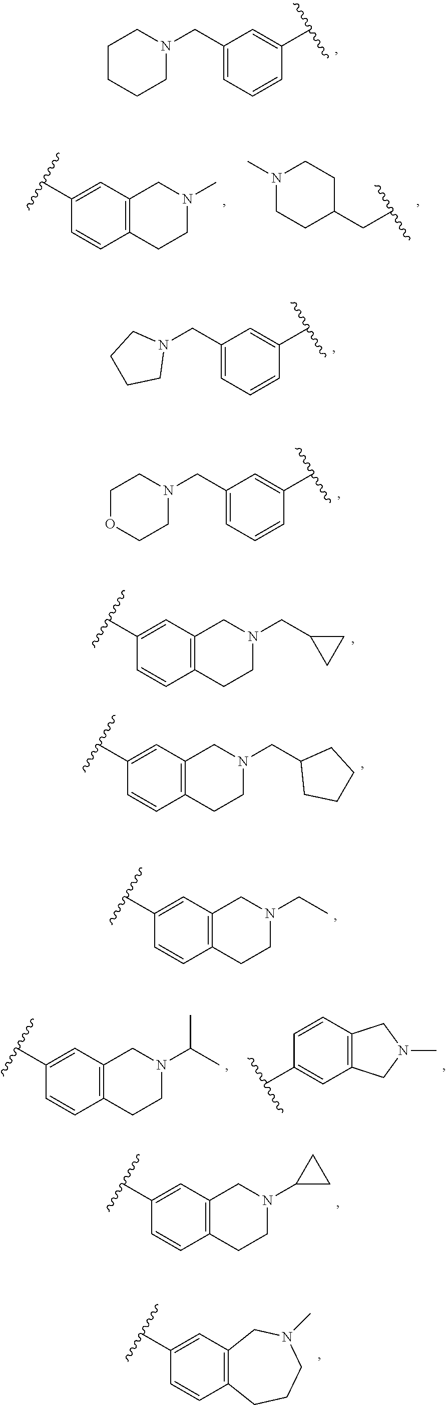

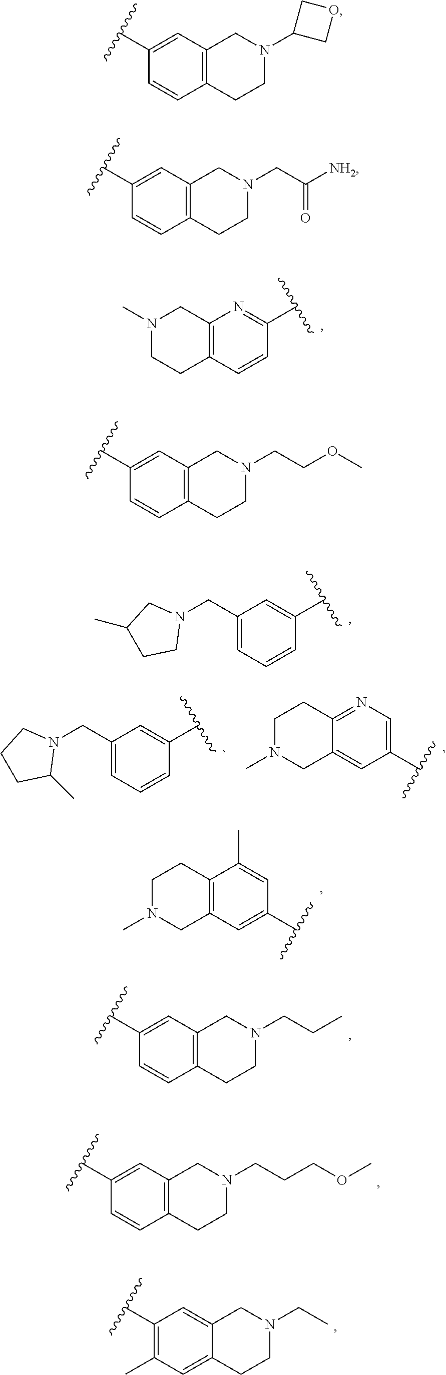

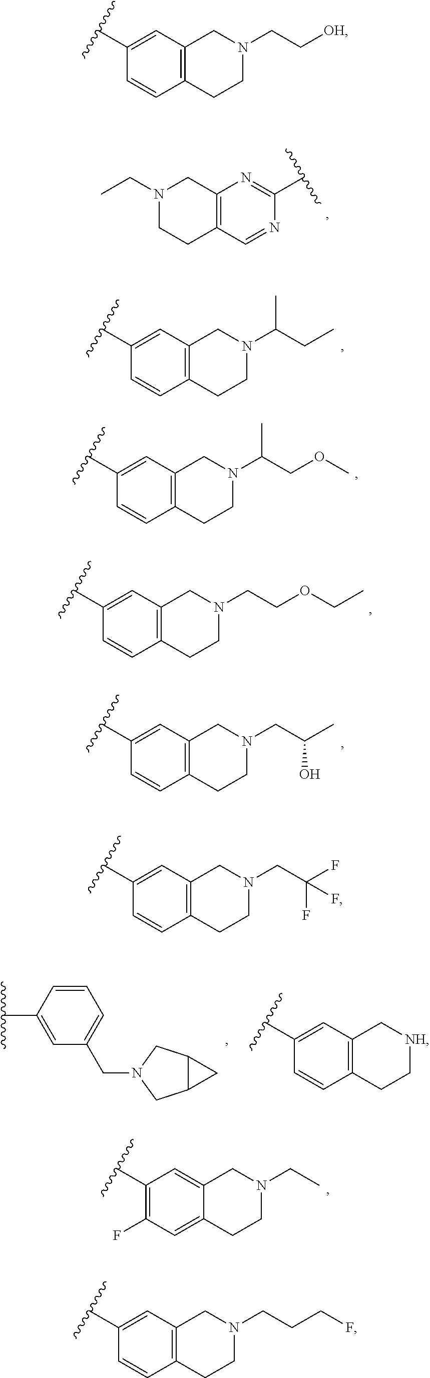

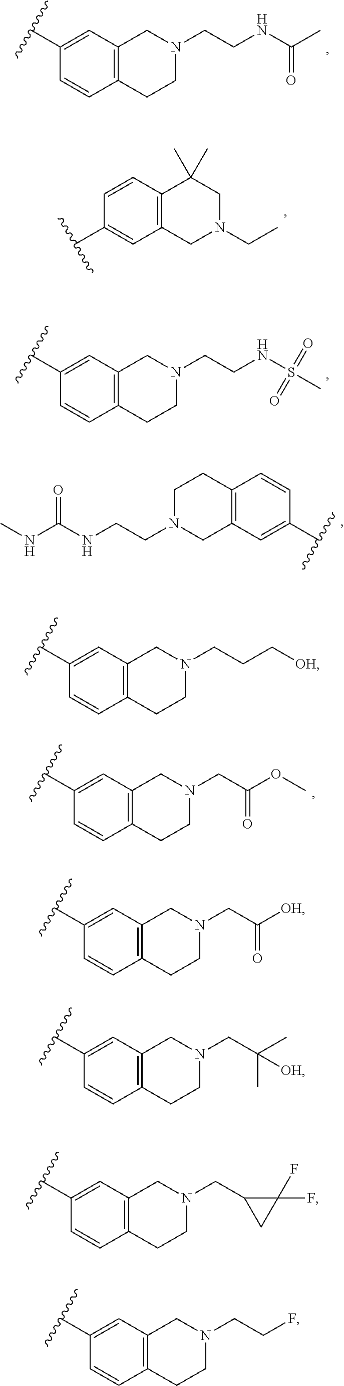

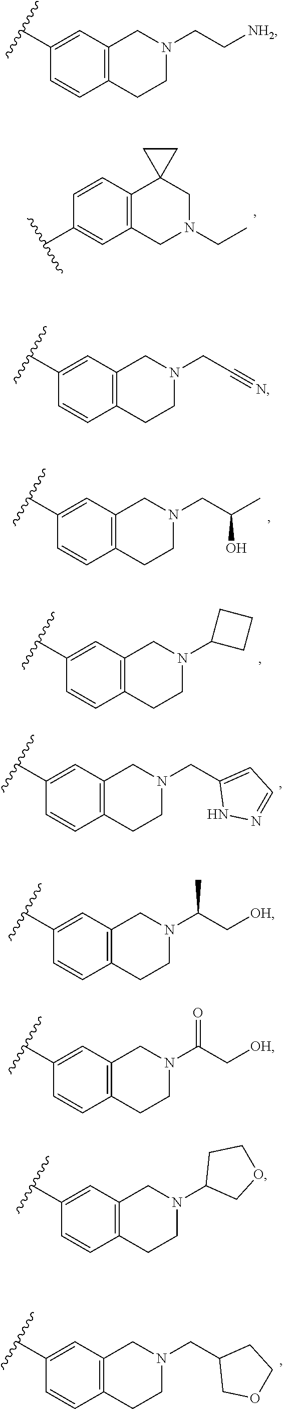

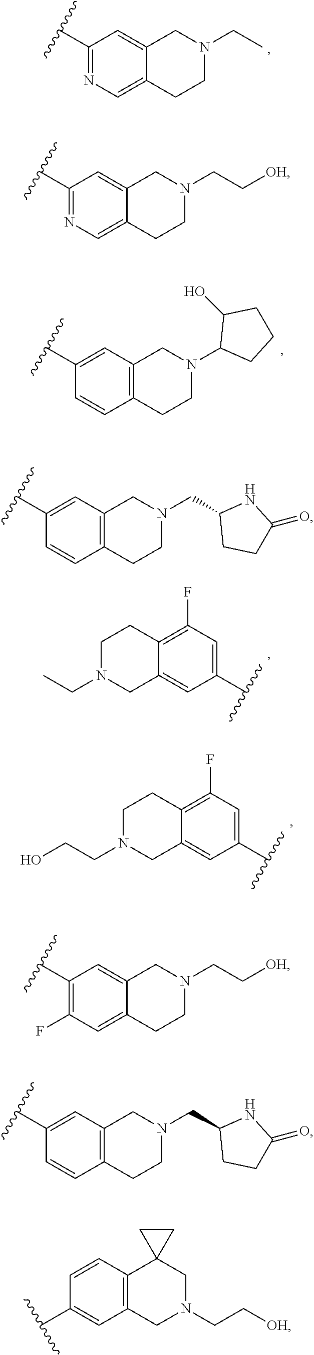

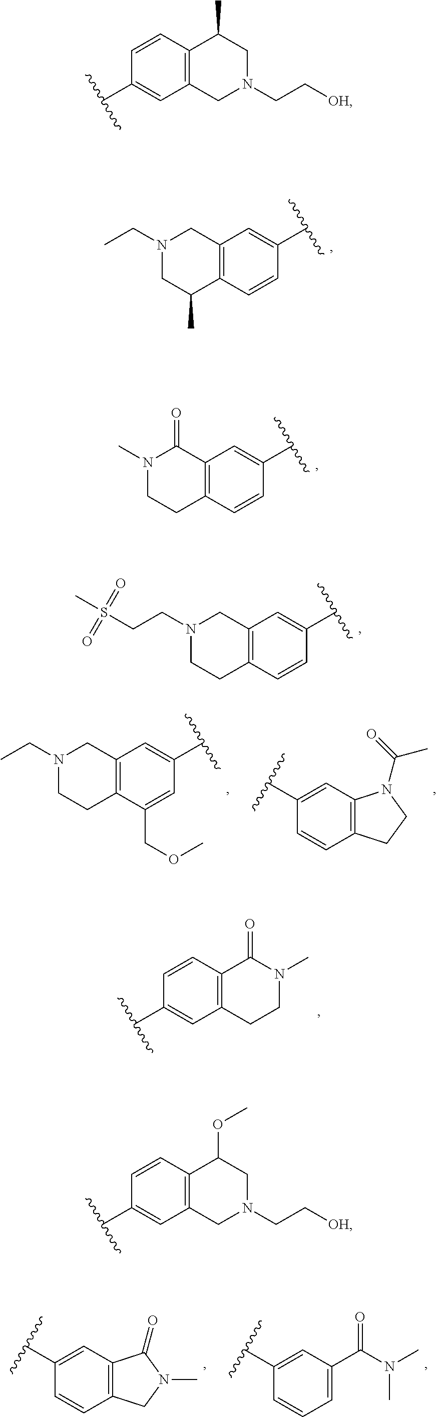

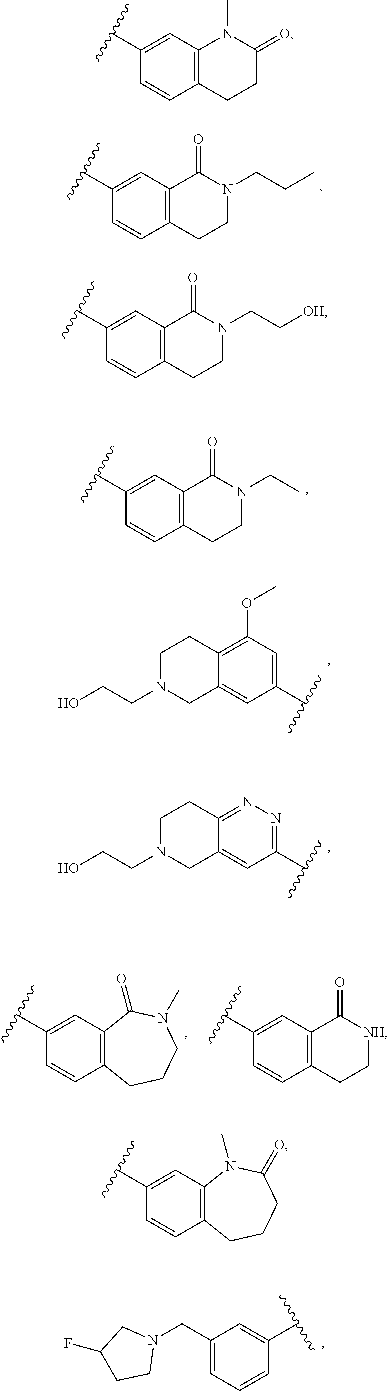

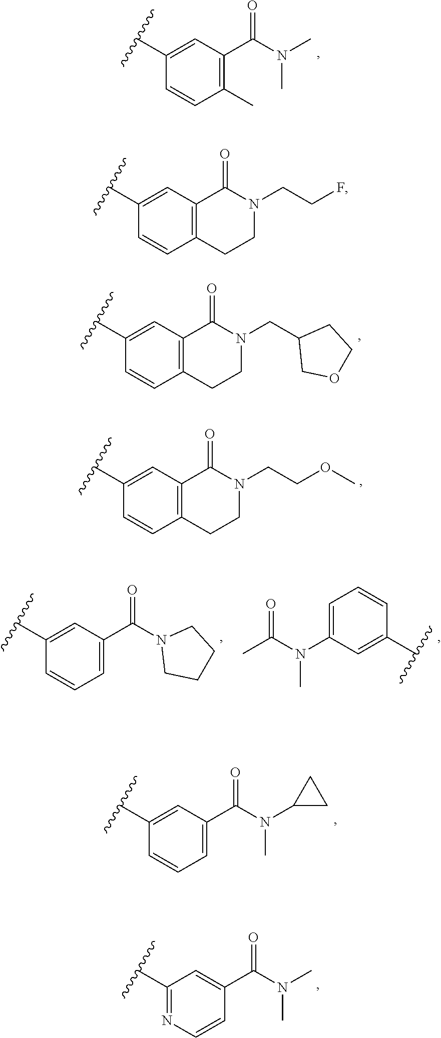

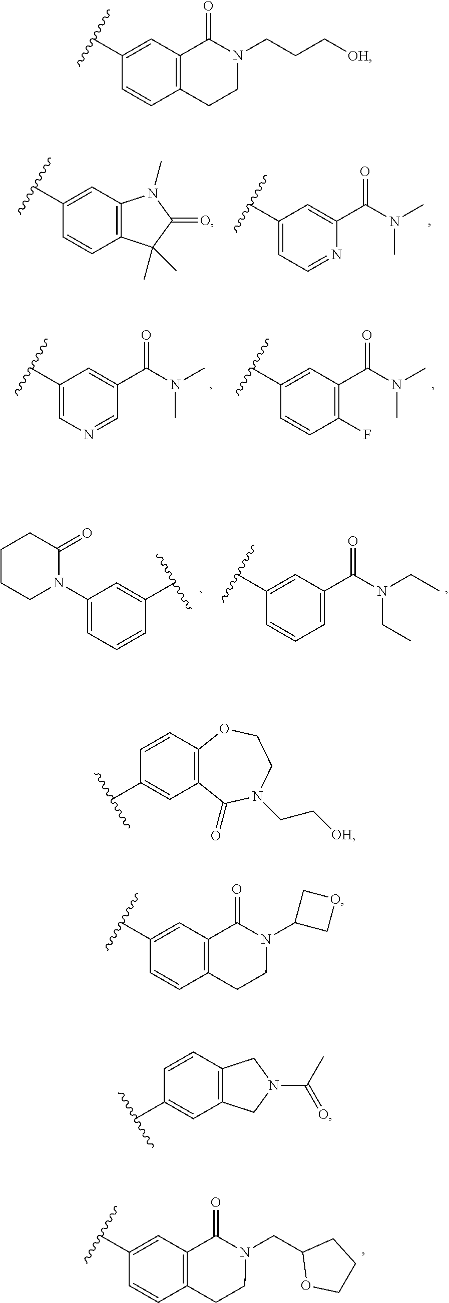

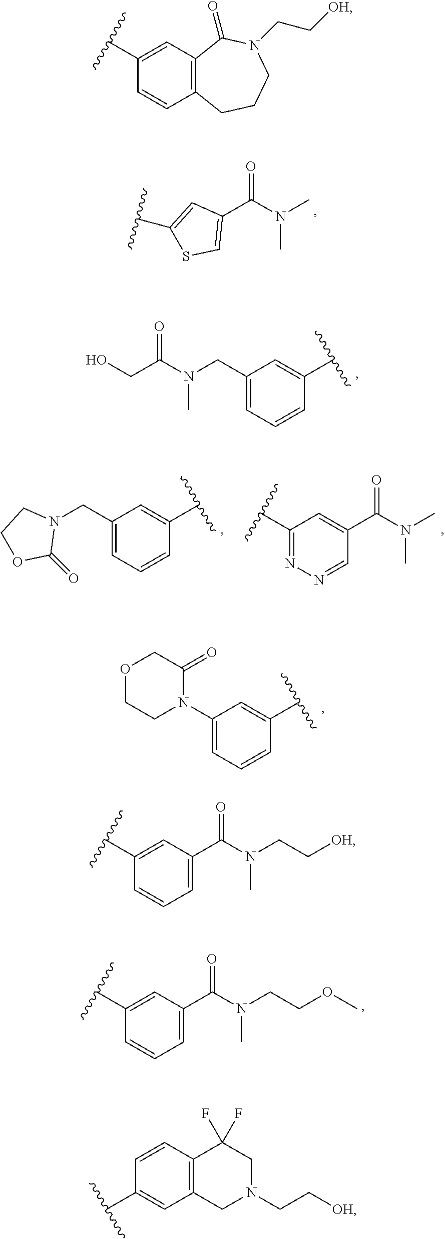

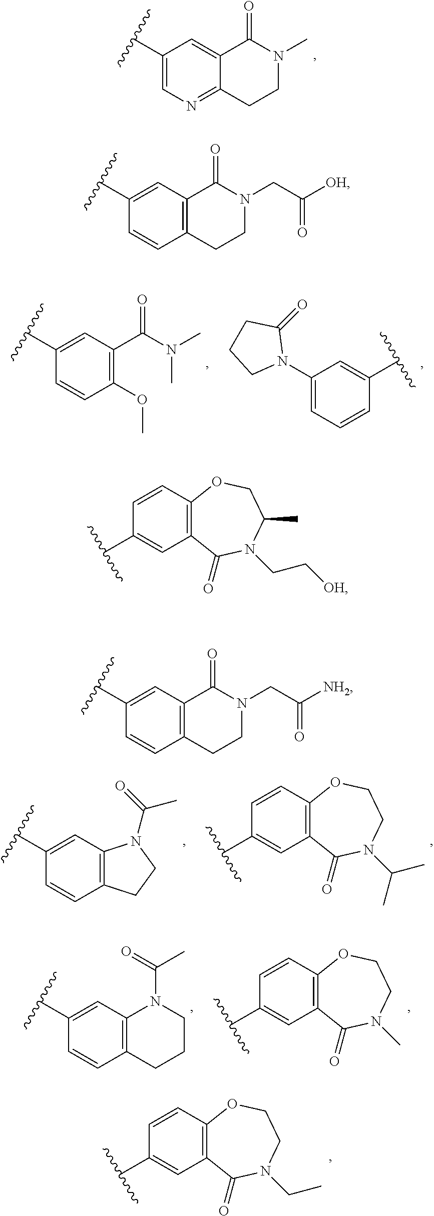

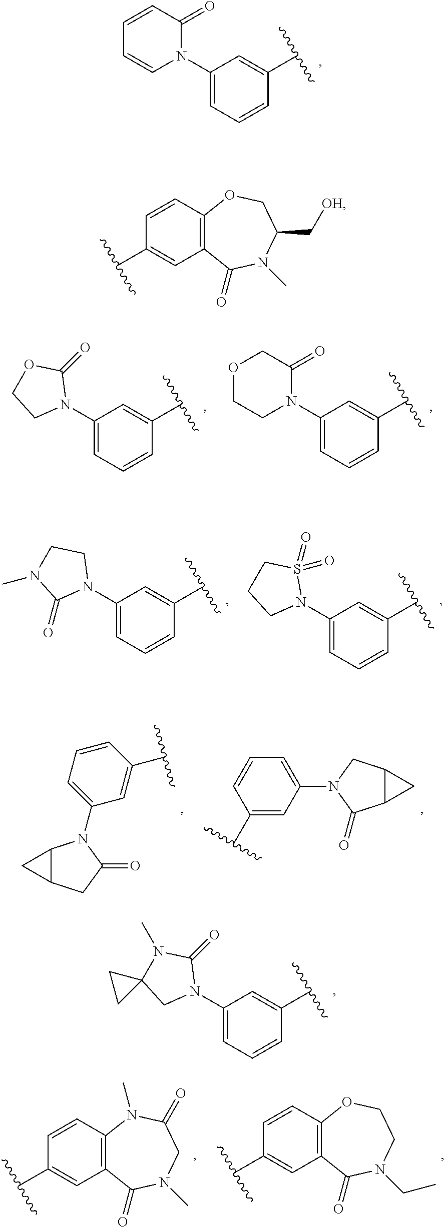

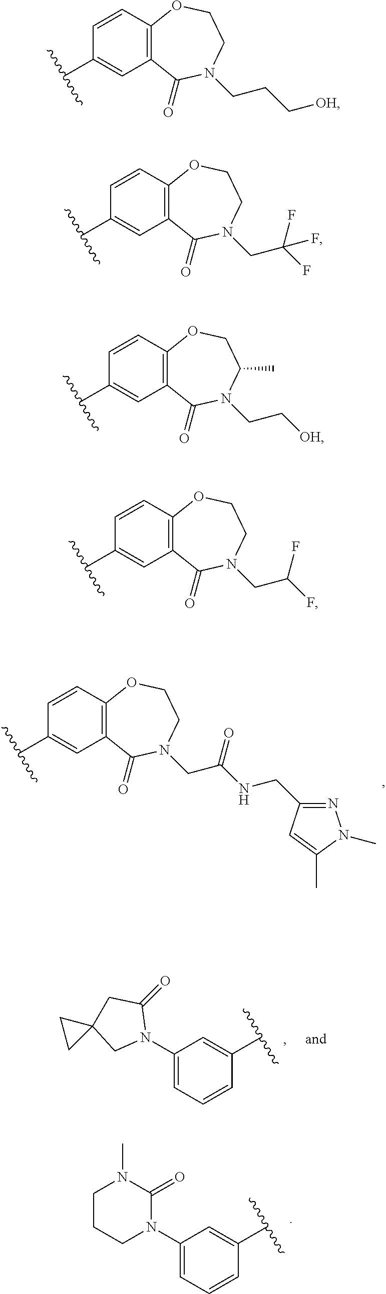

5 R₁ can be selected from:

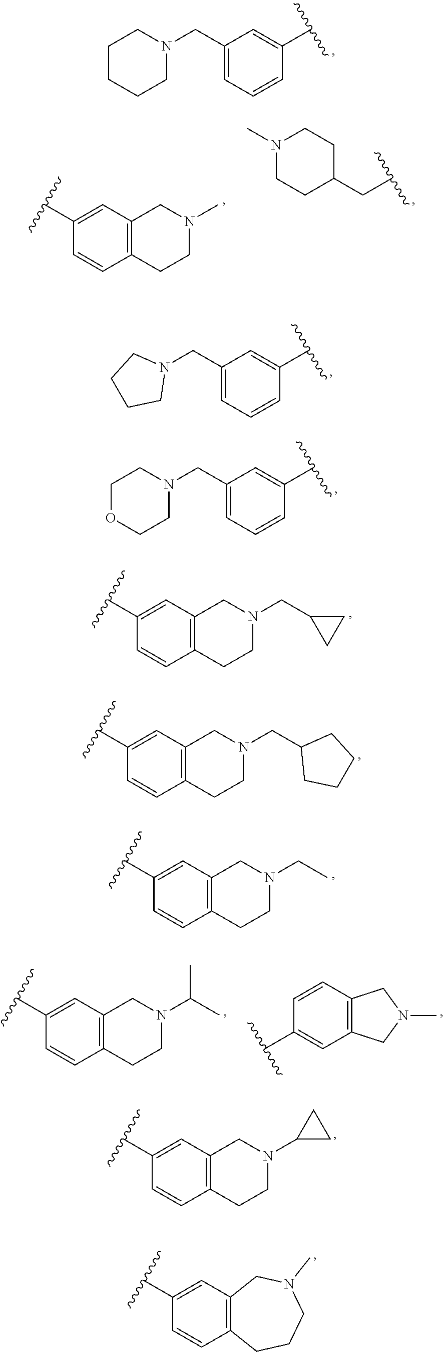

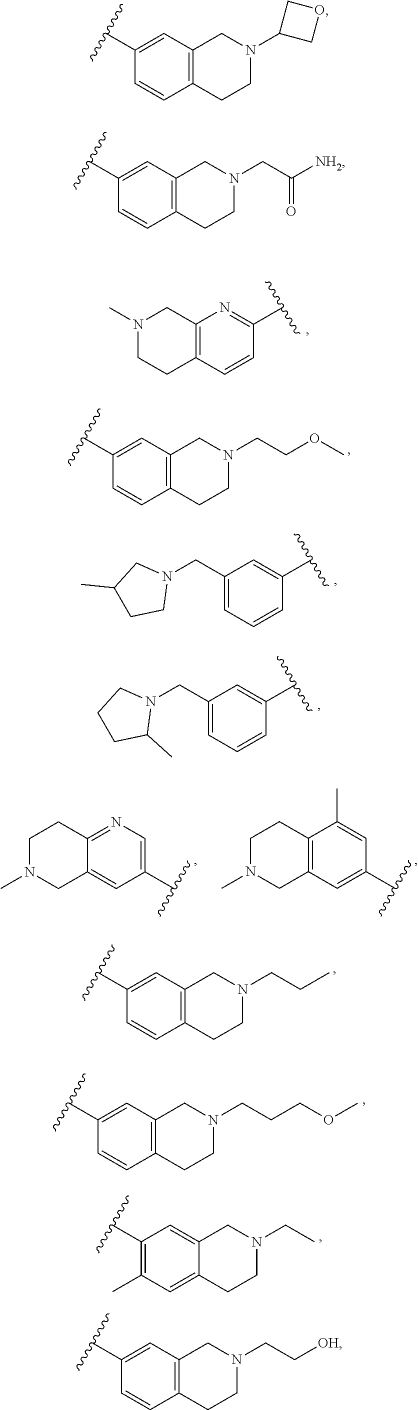

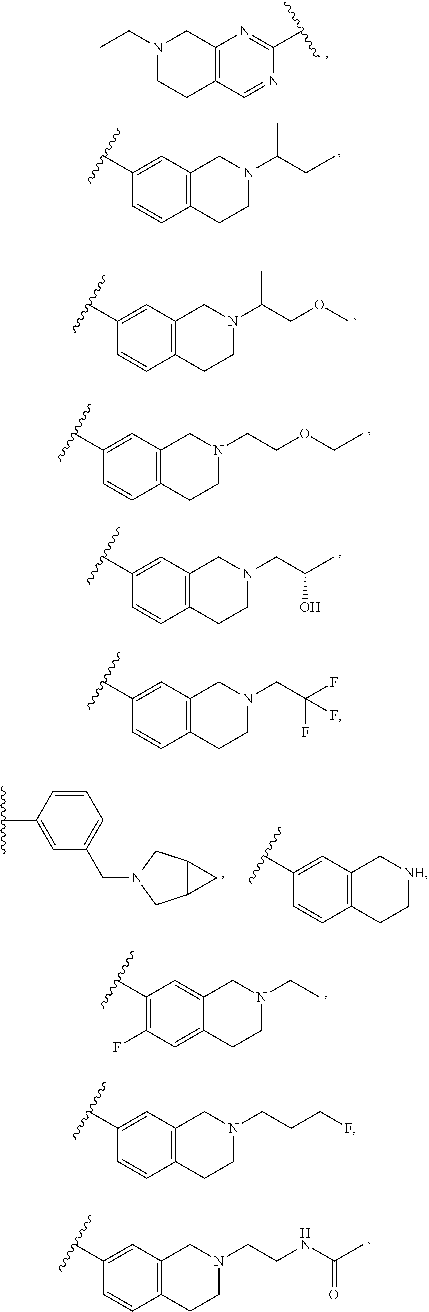

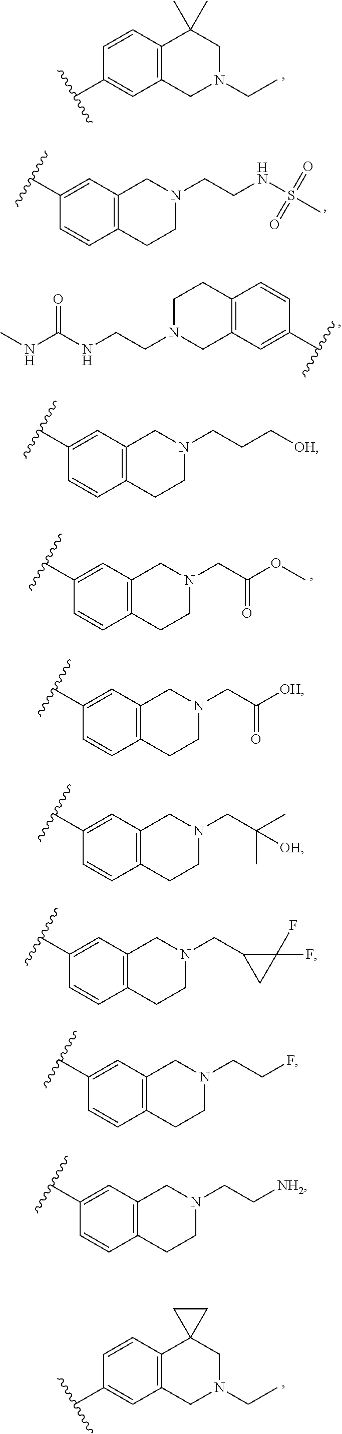

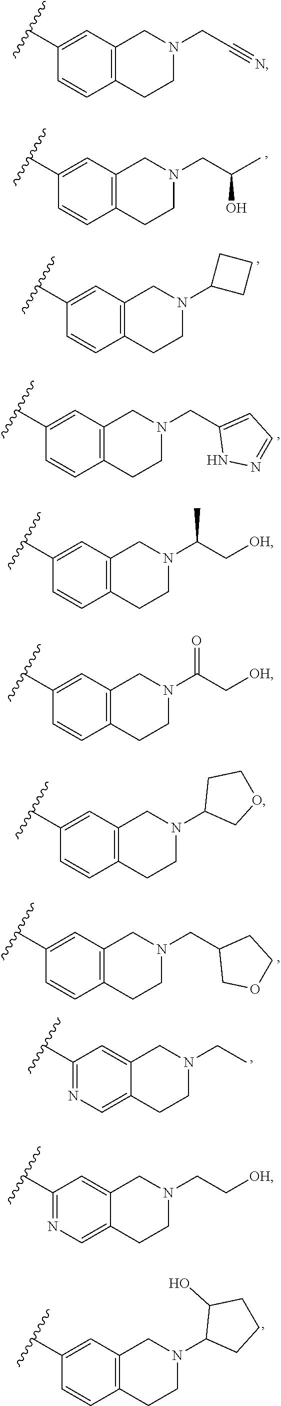

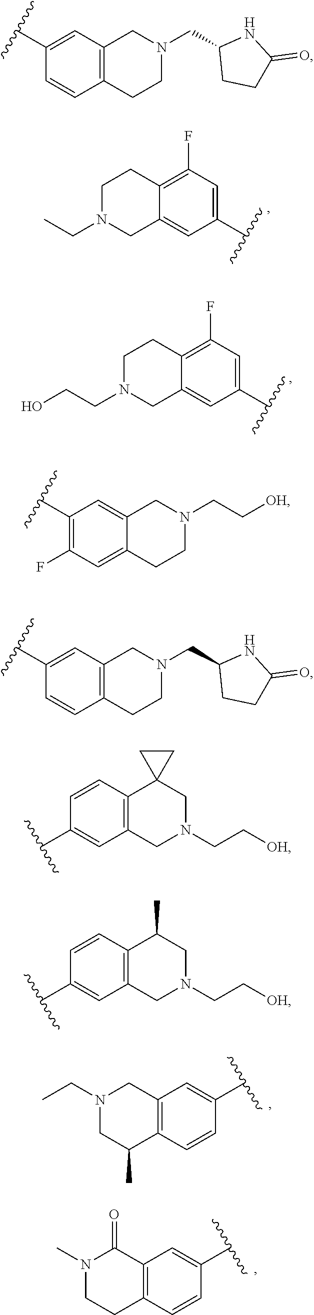

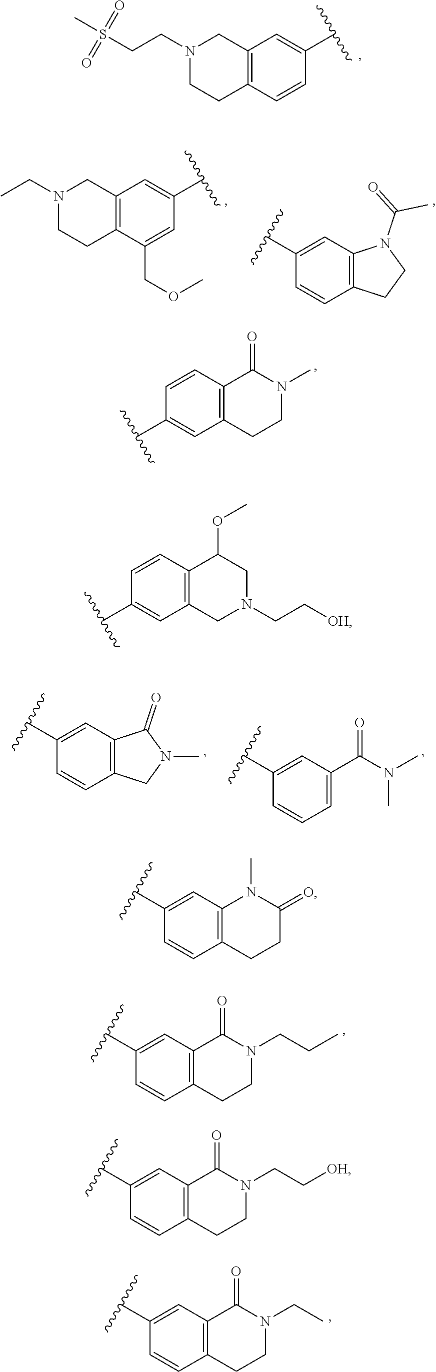

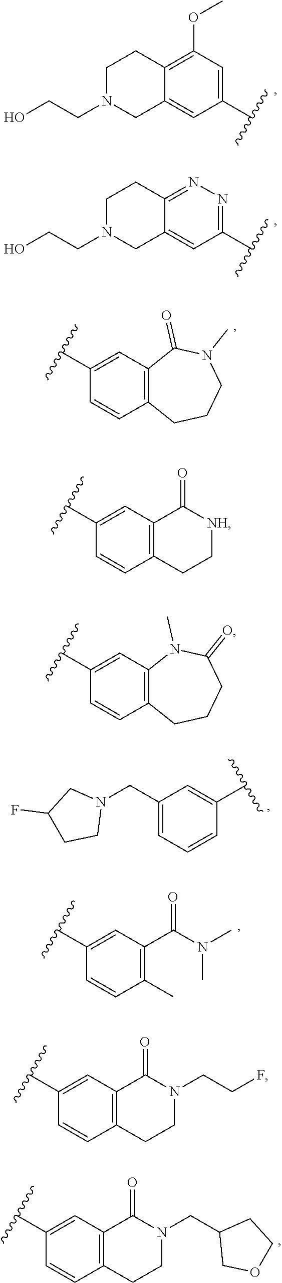

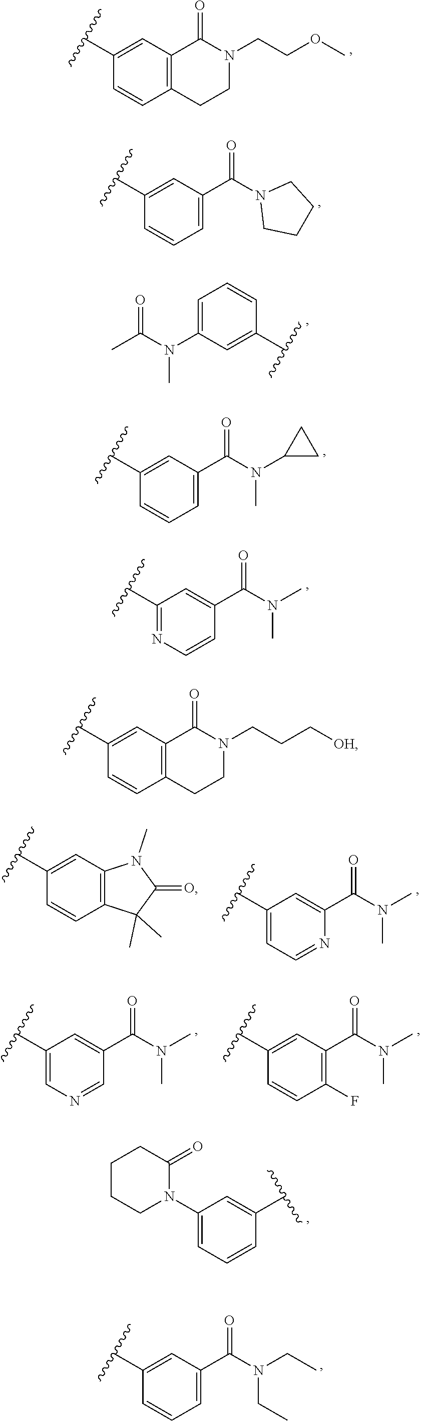

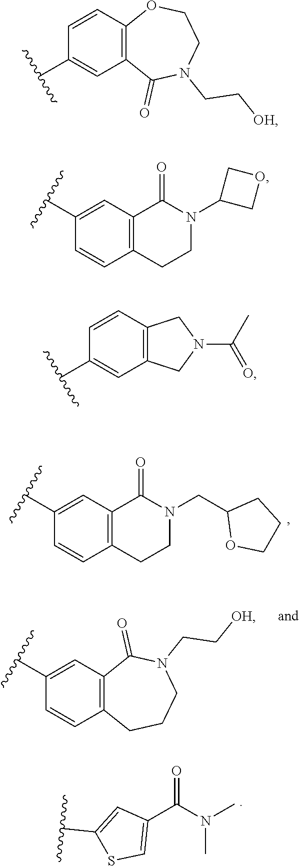

R₁ can be selected from:

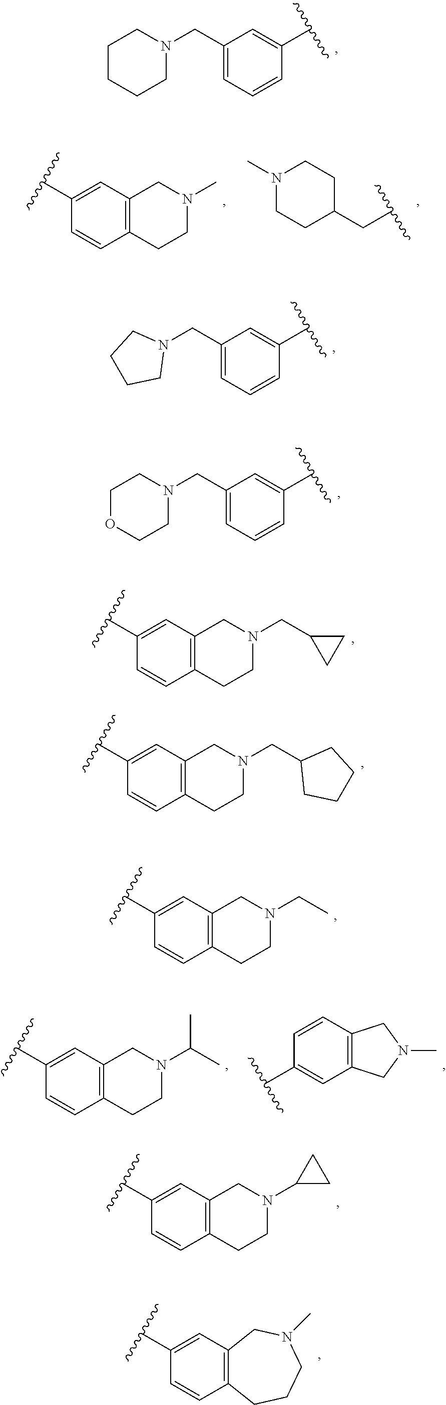

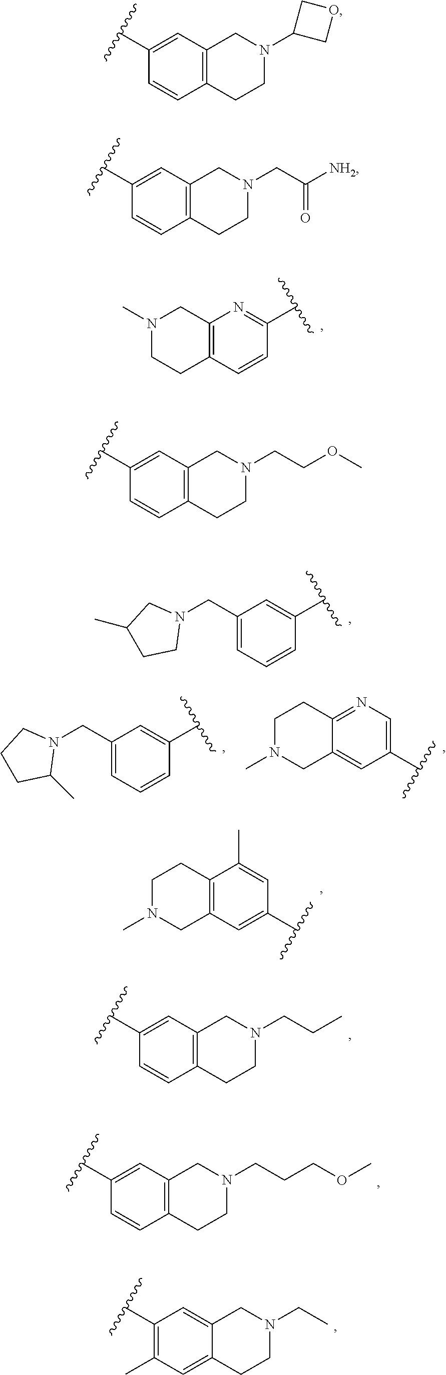

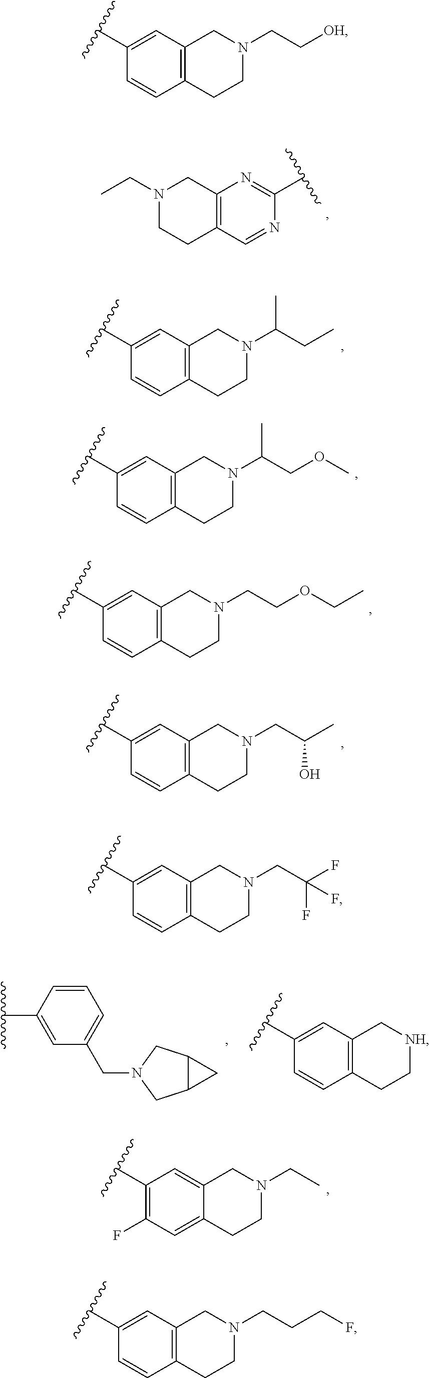

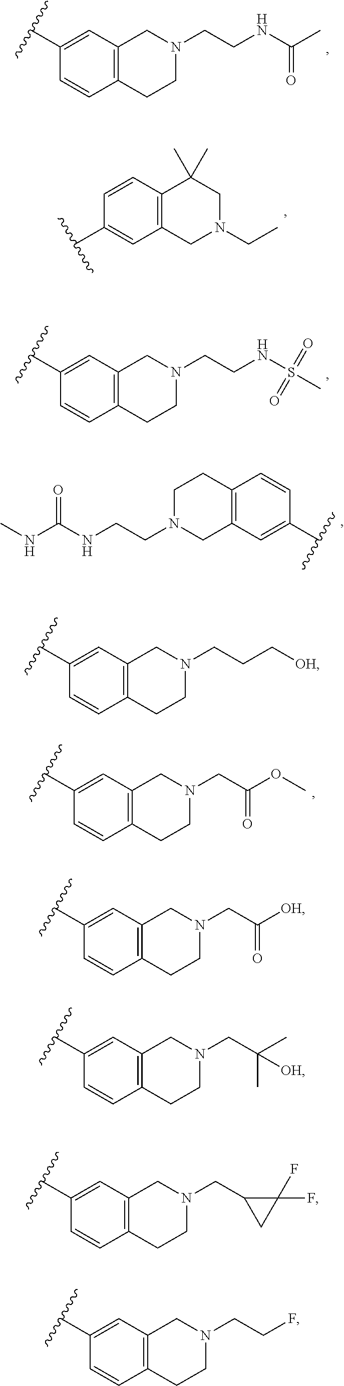

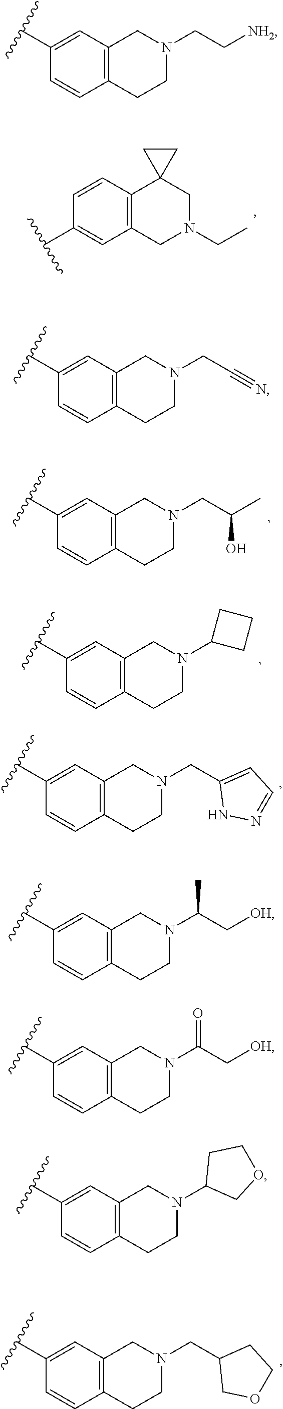

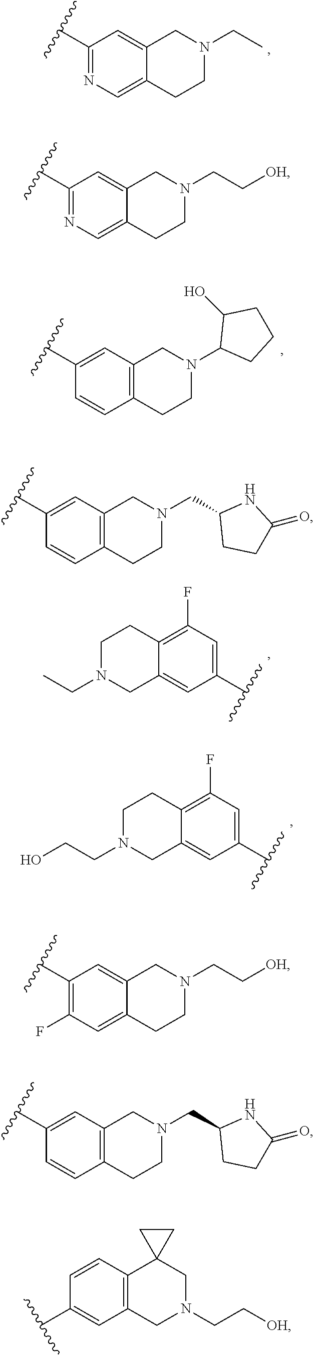

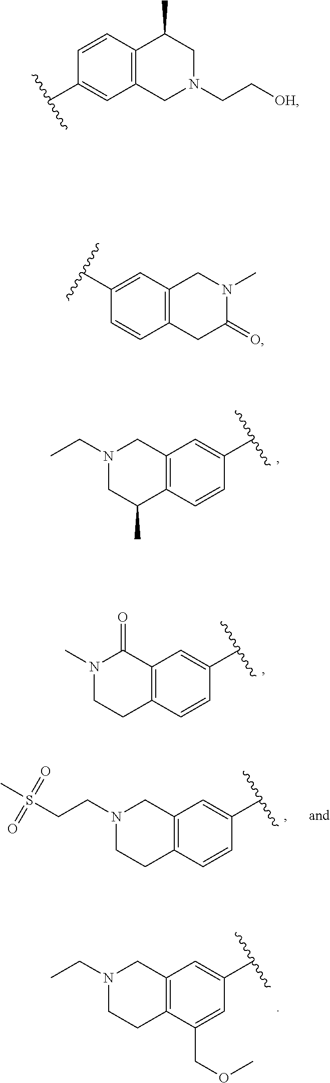 [SMILES: *c1ccc2c(c1)CN(CCO)C[C@@H]2C ; *c1ccc2c(c1)CN(C)C(=O)C2 ; *c1ccc2c(c1)CN(CC)C[C@@H]2C ; *c1ccc2c(c1)C(=O)N(C)CC2 ; *c1ccc2c(c1)CN(CCS(C)(=O)=O)CC2 ; *c1cc(COC)c2c(c1)CN(CC)CC2]

R₁ can be selected from:

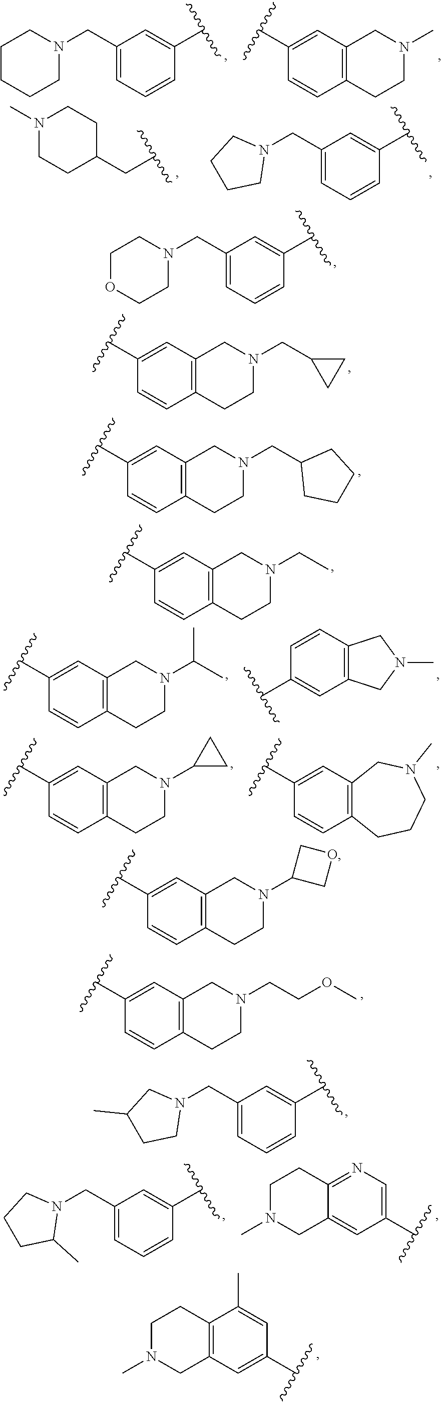

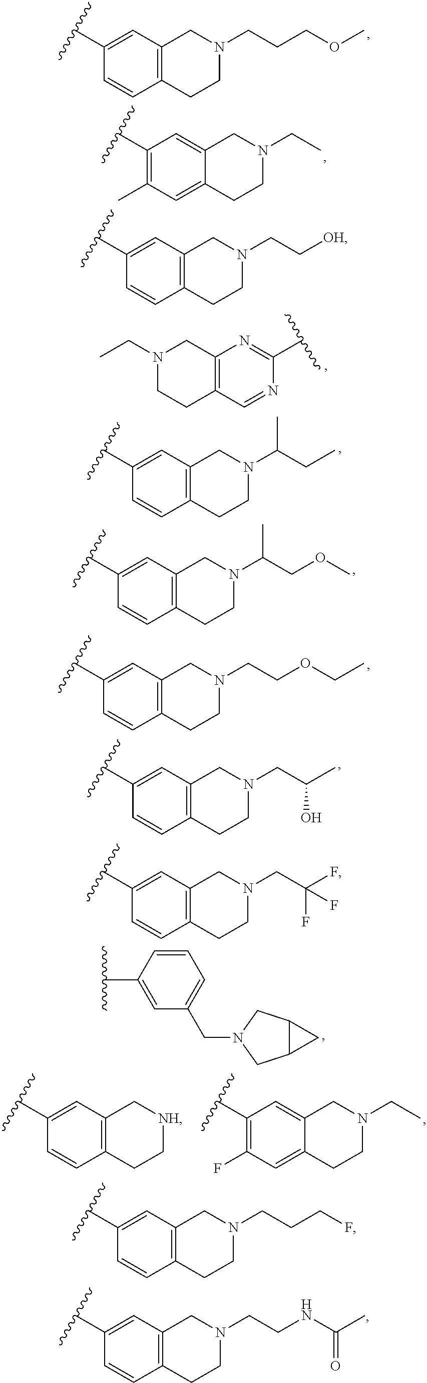

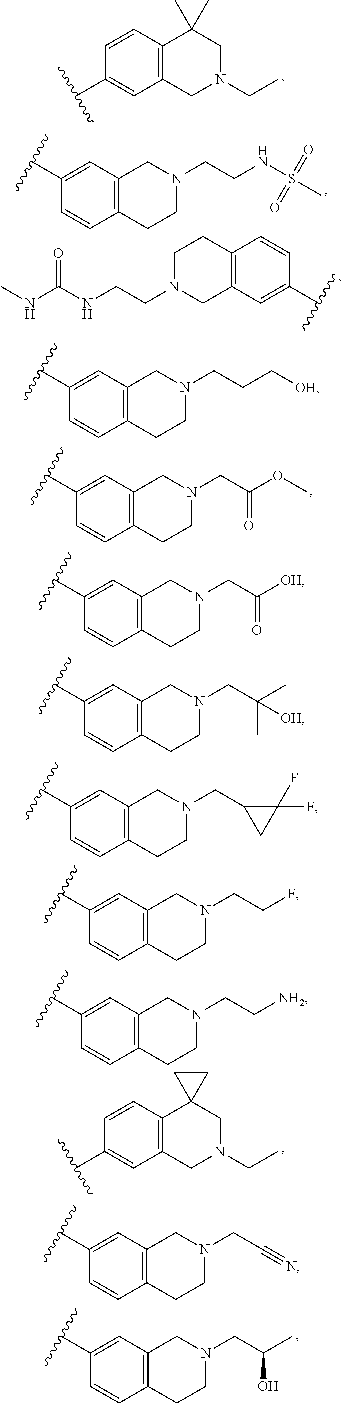

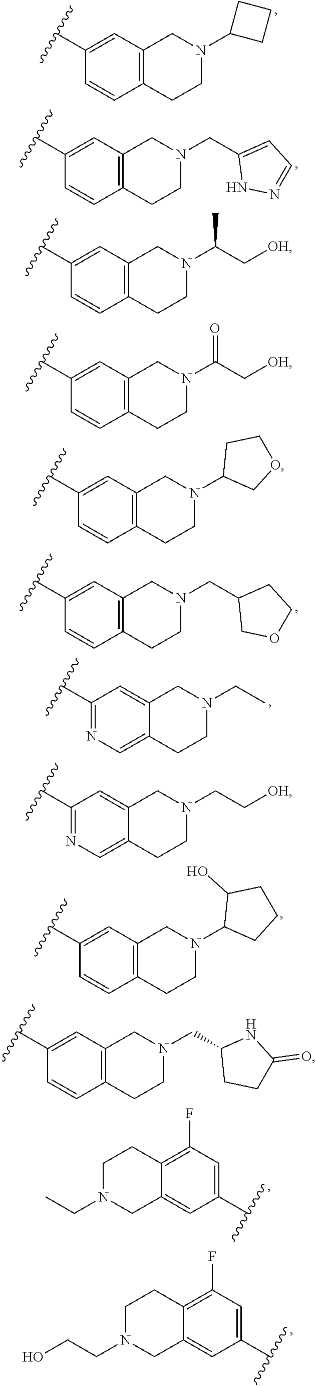

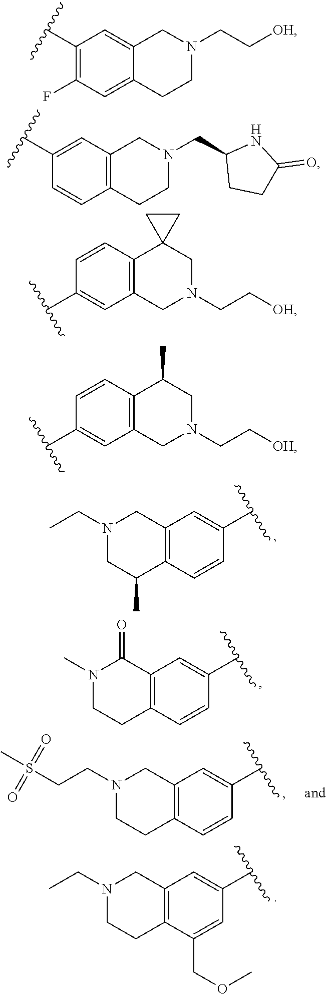

Preferably, R₁ can be selected from:

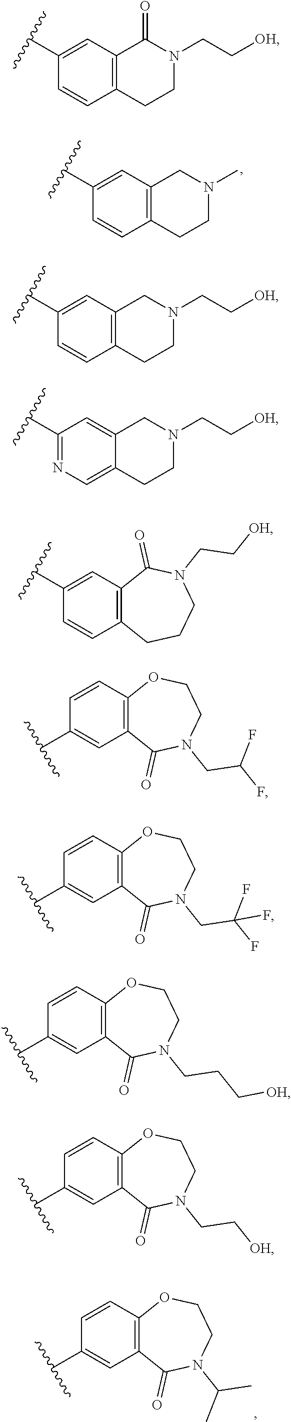

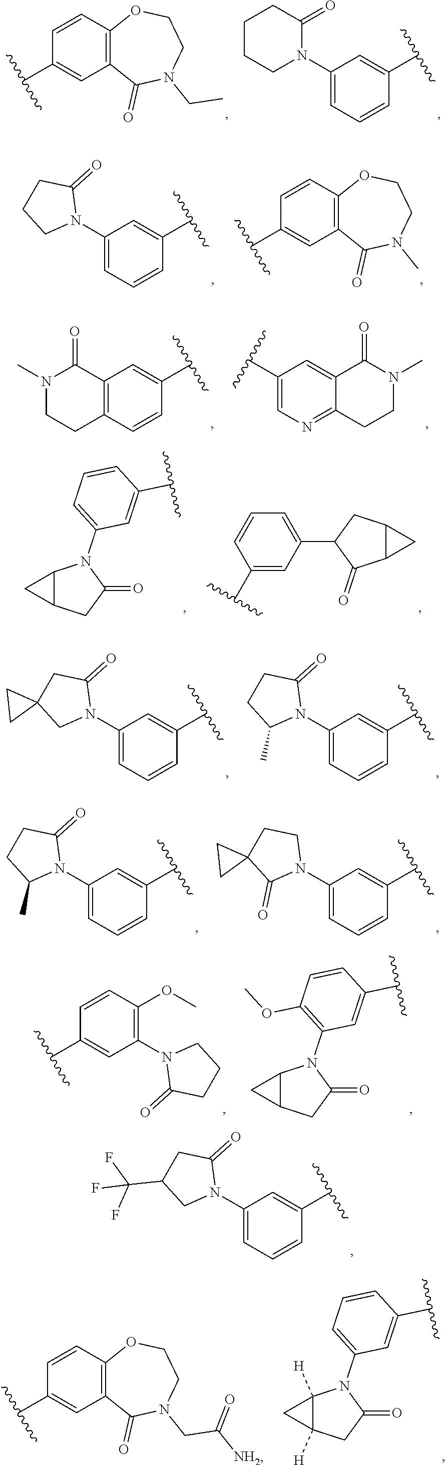

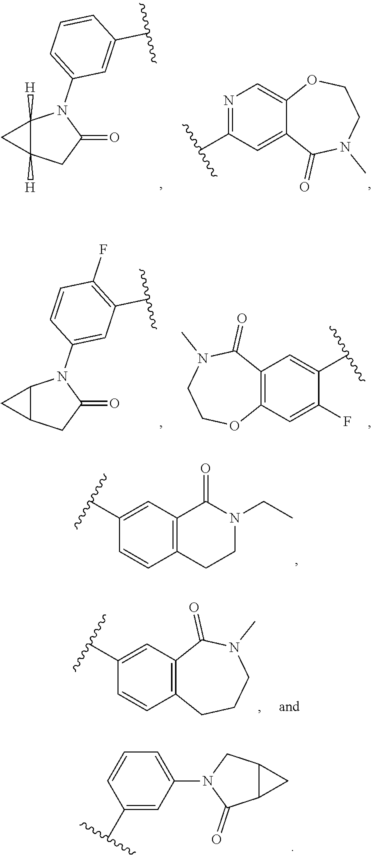

More preferably, R₁ can be selected from:

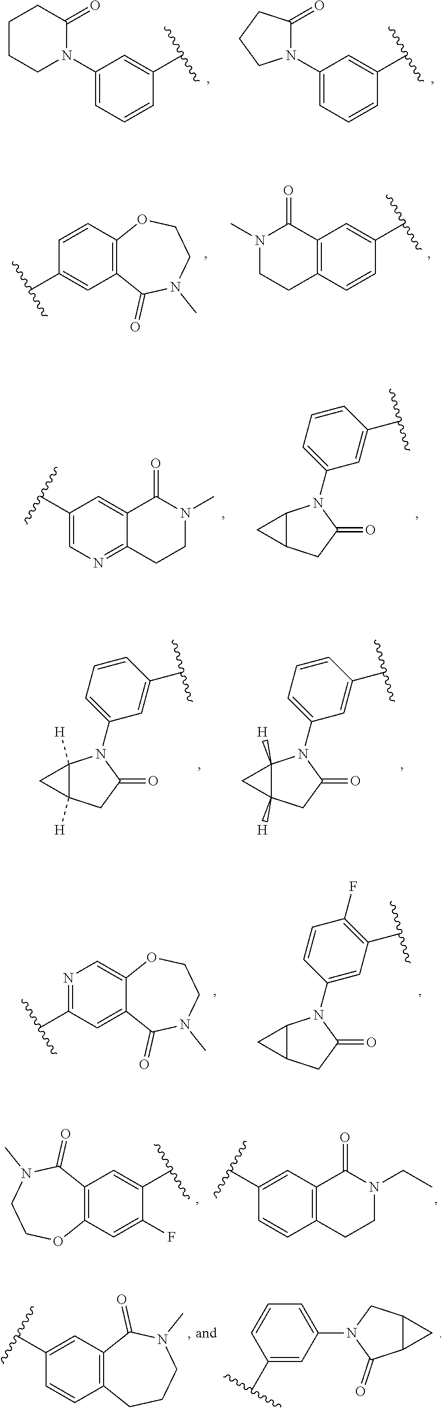

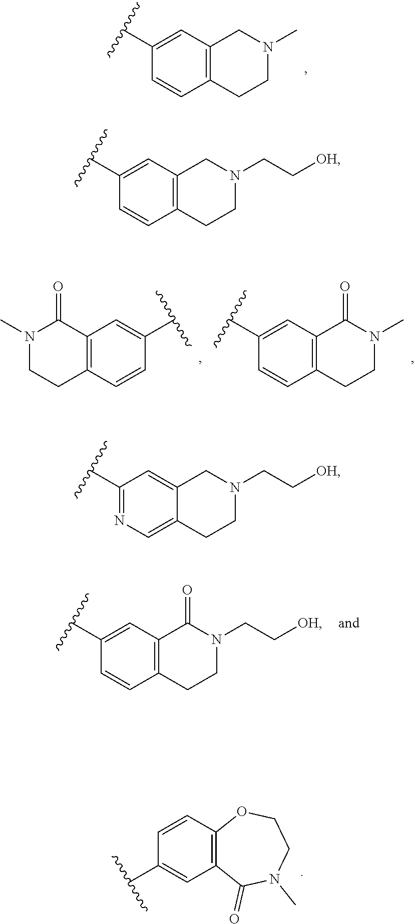

R₁ can be selected from

Most preferably, R₁ can be selected from

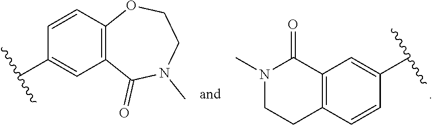 [SMILES: *c1ccc2c(c1)C(=O)N(C)CCO2 ; *c1ccc2c(c1)C(=O)N(C)CC2]

R₁ can be

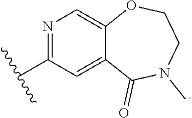 [SMILES: *c1cc2c(cn1)OCCN(C)C2=O]

R₁ can be

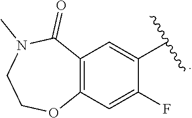 [SMILES: *c1cc2c(cc1F)OCCN(C)C2=O]

R₁ can be selected from:

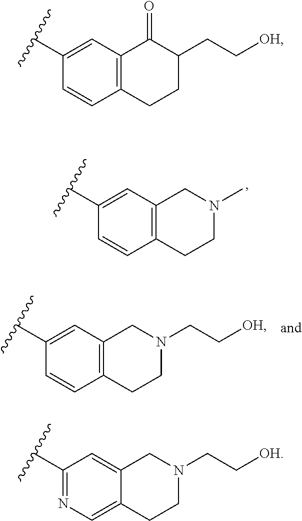 [SMILES: *c1ccc2c(c1)C(=O)C(CCO)CC2 ; *c1ccc2c(c1)CN(C)CC2 ; *c1ccc2c(c1)CN(CCO)CC2 ; *c1cc2c(cn1)CCN(CCO)C2]

R₁ can be selected from:

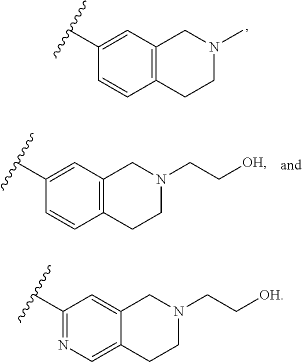 [SMILES: *c1ccc2c(c1)CN(C)CC2 ; *c1ccc2c(c1)CN(CCO)CC2 ; *c1cc2c(cn1)CCN(CCO)C2]

B₁ can be a carbon centre and B₂ can be a carbon centre, wherein B₃ is a single bond between B₂ and the nitrogen atom to which R₁ is attached and B₁ is connected to B₂ via a single bond, or B₁ can be a nitrogen centre and B₂ can be a carbon centre wherein B₁ is connected to B₂ via a double bond; and

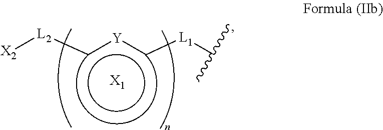

wherein:
n is 1;
L₁ is a linker selected from a single bond and —(CH₂)₁₋₃—;
Y is selected from C, N, O and S; X₁ is phenyl or a 5- or 6-membered carbon-containing aromatic ring containing one, two or three ring members that are selected from N, S, and O;
wherein X₁ may be optionally substituted with 1 or 2 substituents independently selected from alkyl, cycloalkyl, alkoxy, OH, O(cyclopropyl), CF₃, and halo; L₂ is a linker selected from a single bond, —(CH₂)₁₋₃—, and —C(═O)—; and
X₂ is either:
(a) —NR₂OR₂₁, wherein R₂₀ is selected from H, alkyl, and —(CH₂)₀₋₃-R_{22;}
wherein R₂₁ is selected from H, alkyl, and —(CH₂)₀₋₃-R₂₃;

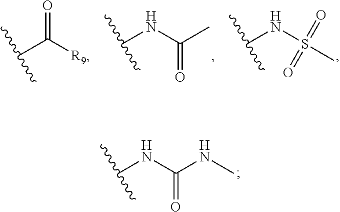

wherein R₂₂ is selected from cycloalkyl, heterocycloalkyl, aryl, heteroaryl,

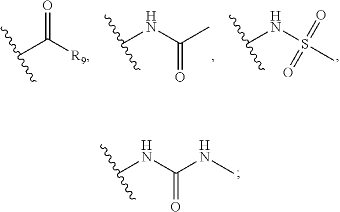

wherein R₂₃ is selected from cycloalkyl, heterocycloalkyl, aryl, heteroaryl, or
(b)₂-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected from alkyl, cycloalkyl, alkoxy, OH, Oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O, and X₂ may be further optionally substituted with 1 or 2 substituents R₂₉ and R₃₀, wherein R₂₉ and R₃₀ are independently selected from alkyl, cycloalkyl, alkoxy, OH, oxo, CF₃, and halo; or
(ii) Formula (III) comprising an aromatic ring A fused to a non-aromatic ring A′,

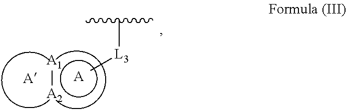

wherein:
L₃ is a single bond;
A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, O(cyclopropyl), CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R₇ is selected from alkyl, alkoxy, and —(CH₂)₀₋₃-R₈,

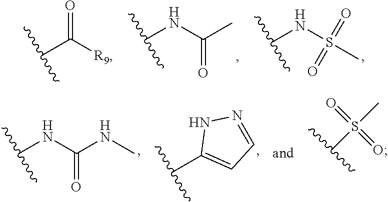

wherein R₈ is selected from cycloalkyl, heterocycloalkyl, wherein R₉ is selected from H, alkyl, OH, N(R₃₁)(R₃₂), and alkoxy;
wherein R₃₁ is H or small alkyl;
wherein R₃₂ is selected from H, small alkyl, and —(CH₂)₀₋₃heteroaryl;
wherein A′ may be optionally substituted with 1, 2, or 3 further substituents R₁₀, R₁₁, and R₁₇, wherein R₁₀, R₁₁, and R₁₇, are independently selected from alkyl, cycloalkyl, alkoxy, OH, oxo, CF₃, and halo, or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O, or wherein A′ may be optionally substituted with 1 or 2 further substituents R₄₃ and R₄₄ independently selected from alkyl, cycloalkyl, alkoxy, OH, oxo, CF₃, and halo, and two different carbon ring members within A′ are connected by a bridging —CH₂-group.
B₄ can be N(R₂), B₅ can be N, and B₆ can be C(R₃), wherein B₄ is connected to B₅ via a single bond and B₅ is connected to B₆ via a double bond, or B₄ can be C(R₂), Bs can be N, and B₆ can be N(R₃), wherein B₄ is connected to B₅ via a double bond and B₅ is connected to B₆ via a single bond; and
R₁ can be:

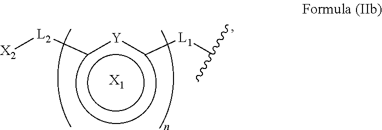

wherein:
n is 1;
L₁ is a linker selected from a single bond and —(CH₂)₁₋₃—;
Y is selected from C, N, O and S;
X₁ is phenyl or a 5- or 6-membered carbon-containing aromatic ring containing one, two or three ring members that are selected from N, S, and O;
wherein X₁ may be optionally substituted with 1 or 2 substituents independently selected from alkyl, cycloalkyl, alkoxy, OH, O(cyclopropyl), CF₃, and halo;
L₂ is a linker selected from a single bond, —(CH₂)₁₋₃—, and —C(═O)—; and
X₂ is either:
(a) —NR₂OR₂₁, wherein R₂₀ is selected from H, alkyl, and —(CH₂)₀₋₃-R_{22;}
wherein R₂₁ is selected from H, alkyl, and —(CH₂)₀₋₃-R₂₃;

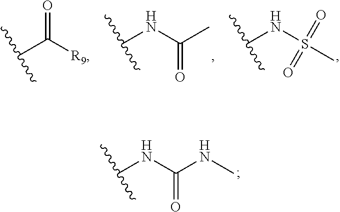

wherein R₂₂ is selected from cycloalkyl, heterocycloalkyl, aryl, heteroaryl,

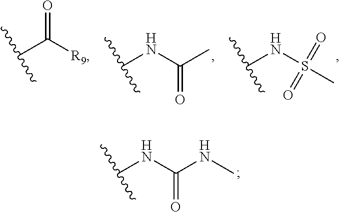

wherein R₂₃ is selected from cycloalkyl, heterocycloalkyl, aryl, heteroaryl, or
(b)₂-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected from alkyl, cycloalkyl, alkoxy, OH, Oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O, and X₂ may be further optionally substituted with 1 or 2 substituents R₂₉ and R₃₀, wherein R₂₉ and R₃₀ are independently selected from alkyl, cycloalkyl, alkoxy, OH, oxo, CF₃, and halo; or
(ii) Formula (III) comprising an aromatic ring A fused to a non-aromatic ring A′,

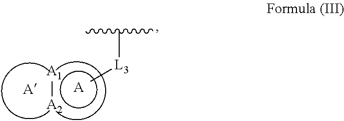

wherein:
L₃ is a single bond;
A₁ and A₂ are both carbon;
A is phenyl or a 5- or 6-membered carbon-containing aromatic ring containing one, two or three
wherein A may be optionally substituted with 1 or 2 substituents independently selected from alkyl, cycloalkyl, alkoxy, OH, O(cyclopropyl), CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R₇ is selected from alkyl, alkoxy, and —(CH₂)₀₋₃-R₈,

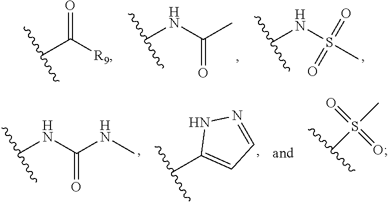

wherein R₈ is selected from cycloalkyl, heterocycloalkyl, wherein R₉ is selected from H, alkyl, OH, N(R₃₁)(R₃₂), and alkoxy;
wherein R₃₁ is H or small alkyl;
wherein R₃₂ is selected from H, small alkyl, and —(CH₂)₀₋₃heteroaryl;
wherein A′ may be optionally substituted with 1, 2, or 3 further substituents R₁₀, R₁₁, and R₁₇, wherein R₁₀, R₁₁, and R₁₇, are independently selected from alkyl, cycloalkyl, alkoxy, OH, oxo, CF₃, and halo, or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O, or wherein A′ may be optionally substituted with 1 or 2 further substituents R₄₃ and R₄₄ independently selected from alkyl, cycloalkyl, alkoxy, OH, oxo, CF₃, and halo, and two different carbon ring members within A′ are connected by a bridging —CH₂-group.
B₁ can be a carbon centre and B₂ can be a carbon centre, wherein B₃ is a single bond between B₂ and the nitrogen atom to which R₁ is attached and B₁ is connected to B₂ via a single bond, or B₁ can be a nitrogen centre and B₂ can be a carbon centre wherein B₁ is connected to B₂ via a double bond; B₄ can be N(R₂), B₅ can be N, and B₆ can be C(R₃), wherein B₄ is connected to B₅ via a single bond and B₅ is connected to B₆ via a double bond, or B₄ can be C(R₂), B₅ can be N, and B₆ can be N(R₃), wherein B₄ is connected to B₅ via a double bond and B₅ is connected to B₆ via a single bond; and

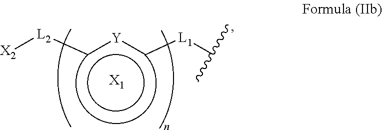

wherein:
n is 1;
L₁ is a linker selected from a single bond and —(CH₂)₁₋₃—;
Y is selected from C, N, O and S; X₁ is phenyl or a 5- or 6-membered carbon-containing aromatic ring containing one, two or three ring members that are selected from N, S, and O;
wherein X₁ may be optionally substituted with 1 or 2 substituents independently selected from alkyl, cycloalkyl, alkoxy, OH, O(cyclopropyl), CF₃, and halo; L₂ is a linker selected from a single bond, —(CH₂)₁₋₃—, and —C(═O)—; and
X₂ is either:
(a) —NR₂OR₂₁, wherein R₂₀ is selected from H, alkyl, and —(CH₂)₀₋₃-R_{22;}
wherein R₂₁ is selected from H, alkyl, and —(CH₂)₀₋₃-R₂₃;

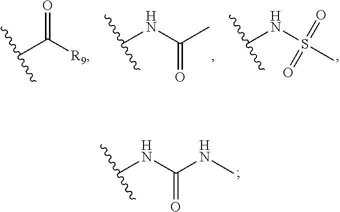

wherein R₂₂ is selected from cycloalkyl, heterocycloalkyl, aryl, heteroaryl,

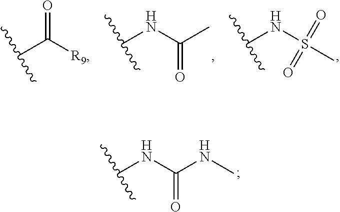

wherein R₂₃ is selected from cycloalkyl, heterocycloalkyl, aryl, heteroaryl, or
(b)₂-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected from alkyl, cycloalkyl, OH, oxo, CF₃, and halo, alkoxy, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O, and X₂ may be further optionally substituted with 1 or 2 substituents R₂₉ and R₃₀, wherein R₂₉ and R₃₀ are independently selected from alkyl, cycloalkyl, alkoxy, OH, oxo, CF₃, and halo; or
(ii) Formula (III) comprising an aromatic ring A fused to a non-aromatic ring A′,

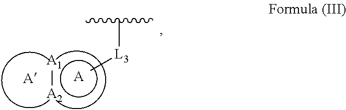

wherein:
L₃ is a single bond;
A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, O(cyclopropyl), CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R₇ is selected from alkyl, alkoxy, and —(CH₂)₀₋₃-R₈,

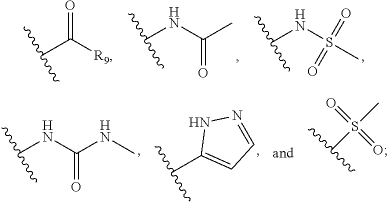

wherein R₈ is selected from cycloalkyl, heterocycloalkyl, wherein R₉ is selected from H, alkyl, OH, N(R₃₁)(R₃₂), and alkoxy;
wherein R₃₁ is H or small alkyl;
wherein R₃₂ is selected from H, small alkyl, and —(CH₂)₀₋₃heteroaryl;
wherein A′ may be optionally substituted with 1, 2, or 3 further substituents R₁₀, R₁₁, and R₁₇, wherein R₁₀, R₁₁, and R₁₇, are independently selected from alkyl, cycloalkyl, alkoxy, OH, oxo, CF₃, and halo, or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O, or wherein A′ may be optionally substituted with 1 or 2 further substituents R₄₃ and R₄₄ independently selected from alkyl, cycloalkyl, alkoxy, OH, oxo, CF₃, and halo, and two different carbon ring members within A′ are connected by a bridging —CH₂-group.
B₁ can be a carbon centre and B₂ can be a carbon centre, wherein B₃ is a single bond between B₂ and the nitrogen atom to which R₁ is attached and B₁ is connected to B₂ via a single bond, or B₁ can be a nitrogen centre and B₂ can be a carbon centre wherein B₁ is connected to B₂ via a double bond; and R₂ can be selected from —C(═O)—N(R₄)(R₅), —C(═O)—O—R₆,—CH₂—NH—C(═O) CH₃,—CH₂—NH—S(═O)₂R₃₅,

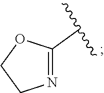 [SMILES: *C1=NCCO1]

—CH₂—O—R₁₆,—CH₂—OH, —CN, and and
R₁ can be:
(i) Formula (IIb),

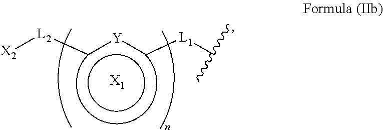

wherein:
n is 1;
L₁ is a linker selected from a single bond and —(CH₂)₁₋₃—;
Y is selected from C, N, O and S; X₁ is phenyl or a 5- or 6-membered carbon-containing aromatic ring containing one, two or three ring members that are selected from N, S, and O;
wherein X₁ may be optionally substituted with 1 or 2 substituents independently selected from alkyl, cycloalkyl, alkoxy, OH, O(cyclopropyl), CF₃, and halo;
L₂ is a linker selected from a single bond, —(CH₂)₁₋₃—, and —C(═O)—; and X₂ is either:
(a) —NR₂OR₂₁, wherein R₂₀ is selected from H, alkyl, and —(CH₂)₀₋₃-R_{22;}
wherein R₂₁ is selected from H, alkyl, and —(CH₂)₀₋₃-R₂₃;

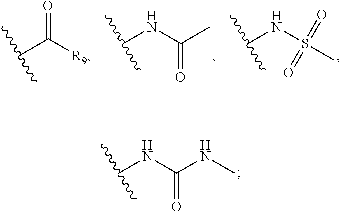

wherein R₂₂ is selected from cycloalkyl, heterocycloalkyl, aryl, heteroaryl,

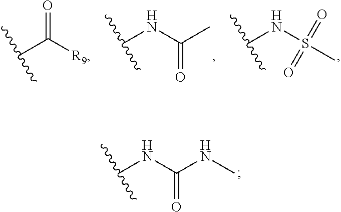

wherein R₂₃ is selected from cycloalkyl, heterocycloalkyl, aryl, heteroaryl, or
(b)₂-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O, and X₂ may be further optionally substituted with 1 or 2 substituents R₂₉ and R₃₀, wherein R₂₉ and R₃₀ are independently selected from alkyl, cycloalkyl, alkoxy, OH, oxo, CF₃, and halo; or
(ii) Formula (III) comprising an aromatic ring A fused to a non-aromatic ring A′,

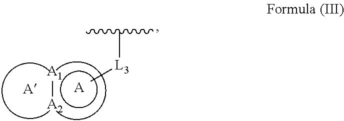

wherein:
L₃ is a single bond;
A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, O(cyclopropyl), CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R₇ is selected from alkyl, alkoxy, and —(CH₂)₀₋₃-R₈,

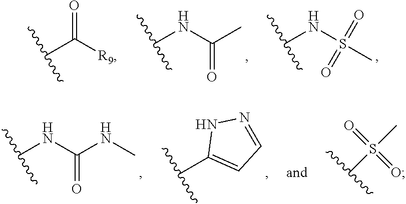

wherein R₈ is selected from cycloalkyl, heterocycloalkyl, wherein R₉ is selected from H, alkyl, OH, N(R₃₁)(R₃₂), and alkoxy;
wherein R₃₁ is H or small alkyl;
wherein R₃₂ is selected from H, small alkyl, and —(CH₂)₀₋₃heteroaryl;
wherein A′ may be optionally substituted with 1, 2, or 3 further substituents R₁₀, R₁₁, and R₁₇, wherein R₁₀, R₁₁, and R₁₇, are independently selected from alkyl, cycloalkyl, alkoxy, OH, oxo, CF₃, and halo, or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O, or wherein A′ may be optionally substituted with 1 or 2 further substituents R₄₃ and R₄₄ independently selected from alkyl, cycloalkyl, alkoxy, OH, oxo, CF₃, and halo, and two different carbon ring members within A′ are connected by a bridging —CH₂-group.
B₄ can be N(R₂), B₅ can be N, and B₆ can be C(R₃), wherein B₄ is connected to B₅ via a single bond and B₅ is connected to B₆ via a double bond, or B₄ can be C(R₂), Bs can be N, and B₆ can be N(R₃), wherein B₄ is connected to B₅ via a double bond and B₅ is connected to B₆ via a single bond; R₂ can be selected from-C(═O)—N(R₄)(R₅), —C(═O)—O—R₆,—CH₂—NH—C(═O) CH₃,—CH₂—NH—S(═O)₂R₃₅,—CH₂—O—R₁₆,—CH₂—OH, —CN, and

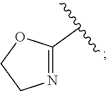 [SMILES: *C1=NCCO1]

and
R₁ can be:

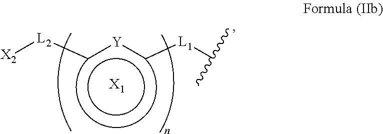

wherein:
n is 1;
L₁ is a linker selected from a single bond and —(CH₂)₁₋₃—; Y is selected from C, N, O and S;
X₁ is phenyl or a 5- or 6-membered carbon-containing aromatic ring containing one, two or three ring members that are selected from N, S, and O;
wherein X₁ may be optionally substituted with 1 or 2 substituents independently selected from alkyl, cycloalkyl, alkoxy, OH, O(cyclopropyl), CF₃, and halo;
L₂ is a linker selected from a single bond, —(CH₂)₁₋₃—, and —C(═O)—; and X₂ is either:
(a) —NR₂OR₂₁, wherein R₂₀ is selected from H, alkyl, and —(CH₂)₀₋₃-R_{22;}
wherein R₂₁ is selected from H, alkyl, and —(CH₂)₀₋₃-R₂₃;

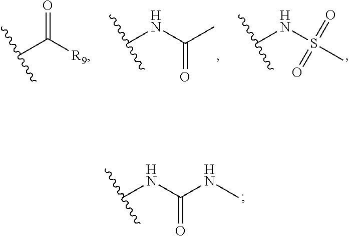

wherein R₂₂ is selected from cycloalkyl, heterocycloalkyl, aryl, heteroaryl,

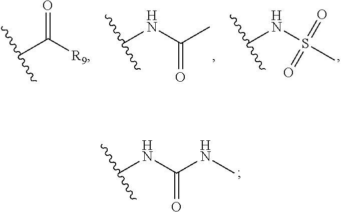

wherein R₂₃ is selected from cycloalkyl, heterocycloalkyl, aryl, heteroaryl, or
(b)₂-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected from alkyl, cycloalkyl, alkoxy, OH, Oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O, and X₂ may be further optionally substituted with 1 or 2 substituents R₂₉ and R₃₀, wherein R₂₉ and R₃₀ are independently selected from alkyl, cycloalkyl, alkoxy, OH, oxo, CF₃, and halo; or
(ii) Formula (III) comprising an aromatic ring A fused to a non-aromatic ring A′,

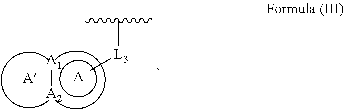

wherein:
L₃ is a single bond;
A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, O(cyclopropyl), CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R₇ is selected from alkyl, alkoxy, and —(CH₂)₀₋₃-R₈,

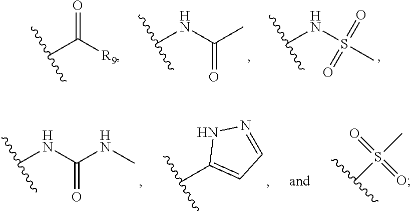

wherein R& is selected from cycloalkyl, heterocycloalkyl, wherein R₉ is selected from H, alkyl, OH, N(R₃₁)(R₃₂), and alkoxy;
wherein R₃₁ is H or small alkyl;
wherein R₃₂ is selected from H, small alkyl, and —(CH₂)₀₋₃heteroaryl;
wherein A′ may be optionally substituted with 1, 2, or 3 further substituents R₁₀, R₁₁, and R₁₇, wherein R₁₀, R₁₁, and R₁₇, are independently selected from alkyl, cycloalkyl, alkoxy, OH, oxo, CF₃, and halo, or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O, or wherein A′ may be optionally substituted with 1 or 2 further substituents R₄₃ and R₄₄ independently selected from alkyl, cycloalkyl, alkoxy, OH, oxo, CF₃, and halo, and two different carbon ring members within A′ are connected by a bridging —CH₂-group.
B₁ can be a carbon centre and B₂ can be a carbon centre, wherein B₃ is a single bond between B₂ and the nitrogen atom to which R₁ is attached and B₁ is connected to B₂ via a single bond, or B₁ can be a nitrogen centre and B₂ can be a carbon centre wherein B₁ is connected to B₂ via a double bond; B₄ can be N(R₂), Bs can be N, and B₆ can be C(R₃), wherein B₄ is connected to B₅ via a single bond and B₅ is connected to B₆ via a double bond, or B₄ can be C(R₂), B₅ can be N, and B₆ can be N(R₃), wherein B₄ is connected to B₅ via a double bond and B₅ is connected to B₆ via a single bond; R₂ can be selected from —C(═O)—N(R₄)(R₅),

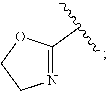 [SMILES: *C1=NCCO1]

—C(═O)—O—R₆,—CH₂—NH—S(═O)₂R₃₅,—CH₂—NH—C(═O) CH₃,—CH₂—O—R₁₆,—CH₂—OH, —CN, and and
R₁ can be:
(i) Formula (IIb),

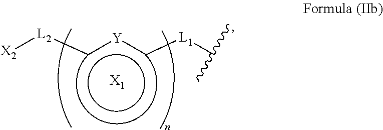

wherein:
n is 1;
L₁ is a linker selected from a single bond and —(CH₂)₁₋₃—;
Y is selected from C, N, O and S; X₁ is phenyl or a 5- or 6-membered carbon-containing aromatic ring containing one, two or three
wherein X₁ may be optionally substituted with 1 or 2 substituents independently selected from alkyl, cycloalkyl, alkoxy, OH, O(cyclopropyl), CF₃, and halo; L₂ is a linker selected from a single bond, —(CH₂)₁₋₃—, and —C(═O)—; and
X₂ is either:
(a) —NR₂OR₂₁, wherein R₂₀ is selected from H, alkyl, and —(CH₂)₀₋₃-R_{22;}
wherein R₂₁ is selected from H, alkyl, and —(CH₂)₀₋₃-R₂₃;

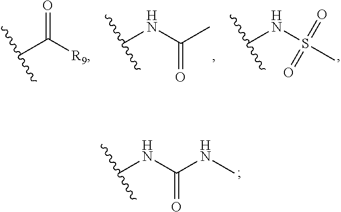

wherein R₂₂ is selected from cycloalkyl, heterocycloalkyl, aryl, heteroaryl,

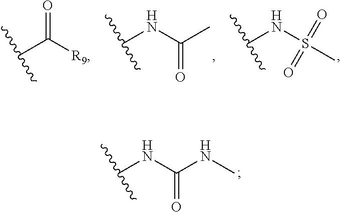

wherein R₂₃ is selected from cycloalkyl, heterocycloalkyl, aryl, heteroaryl, or
(b)₂-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O, and X₂ may be further optionally substituted with 1 or 2 substituents R₂₉ and R₃₀, wherein R₂₉ and R₃₀ are independently selected from alkyl, cycloalkyl, alkoxy, OH, oxo, CF₃, and halo; or

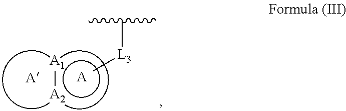

wherein:
L₃ is a single bond;
A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, O(cyclopropyl), CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R₇ is selected from alkyl, alkoxy, and —(CH₂)₀₋₃-R₈,

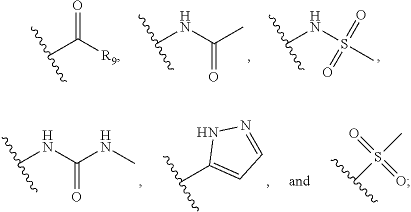

wherein R₈ is selected from cycloalkyl, heterocycloalkyl, wherein R₉ is selected from H, alkyl, OH, N(R₃₁)(R₃₂), and alkoxy;
wherein R₃₁ is H or small alkyl;
wherein R₃₂ is selected from H, small alkyl, and —(CH₂)₀₋₃heteroaryl;
wherein A′ may be optionally substituted with 1, 2, or 3 further substituents R₁₀, R₁₁, and R₁₇, wherein R₁₀, R₁₁, and R₁₇, are independently selected from alkyl, cycloalkyl, alkoxy, OH, oxo, CF₃, and halo, or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O, or wherein A′ may be optionally substituted with 1 or 2 further substituents R₄₃ and R₄₄ independently selected from alkyl, cycloalkyl, alkoxy, OH, oxo, CF₃, and halo, and two different carbon ring members within A′ are connected by a bridging —CH₂-group.
R₂ can be selected from —C(═O)—N(R₄)(R₅), —C(═O)—O—R₆,—CH₂—NH—S(═O)₂R₃₅,—CH₂—NH—C(═O) CH₃,—CH₂—O—R₁₆,

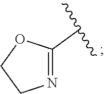 [SMILES: *C1=NCCO1]

—CH₂—OH, —CN, and and
R₁ can be:
(i) Formula (IIb),

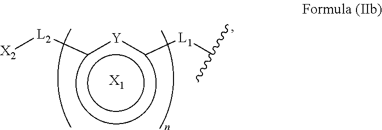

wherein:
n is 1;
L₁ is a linker selected from a single bond and —(CH₂)₁₋₃—;
Y is selected from C, N, O and S; X₁ is phenyl or a 5- or 6-membered carbon-containing aromatic ring containing one, two or three ring members that are selected from N, S, and O;
wherein X₁ may be optionally substituted with 1 or 2 substituents independently selected from alkyl, cycloalkyl, alkoxy, OH, O(cyclopropyl), CF₃, and halo; L₂ is a linker selected from a single bond, —(CH₂)₁₋₃—, and —C(═O)—; and
X₂ is either:
(a) —NR₂OR₂₁, wherein R₂₀ is selected from H, alkyl, and —(CH₂)₀₋₃-R_{22;}
wherein R₂₁ is selected from H, alkyl, and —(CH₂)₀₋₃-R₂₃;

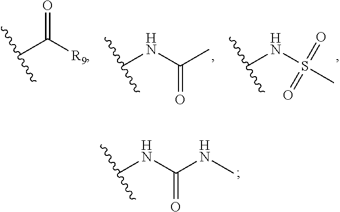

wherein R₂₂ is selected from cycloalkyl, heterocycloalkyl, aryl, heteroaryl,

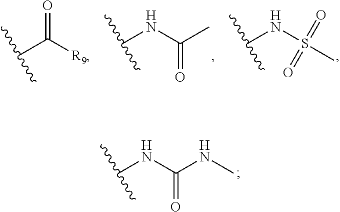

wherein R₂₃ is selected from cycloalkyl, heterocycloalkyl, aryl, heteroaryl, or
(b)₂-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected from alkyl, cycloalkyl, alkoxy, OH, Oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O, and X₂ may be further optionally substituted with 1 or 2 substituents R₂₉ and R₃₀, wherein R₂₉ and R₃₀ are independently selected from alkyl, cycloalkyl, alkoxy, OH, oxo, CF₃, and halo; or
(ii) Formula (III) comprising an aromatic ring A fused to a non-aromatic ring A′,

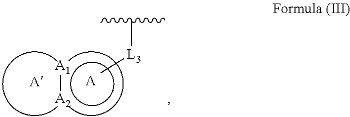

wherein:
L₃ is a single bond;
A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, O(cyclopropyl), CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R₇ is selected from alkyl, alkoxy, and —(CH₂)₀₋₃-R₈,

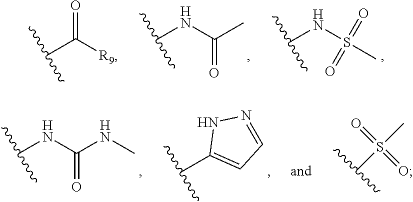

wherein R₈ is selected from cycloalkyl, heterocycloalkyl, wherein R₉ is selected from H, alkyl, OH, N(R₃₁)(R₃₂), and alkoxy;
wherein R₃₁ is H or small alkyl;
wherein R₃₂ is selected from H, small alkyl, and —(CH₂)₀₋₃heteroaryl;
wherein A′ may be optionally substituted with 1, 2, or 3 further substituents R₁₀, R₁₁, and R₁₇, wherein R₁₀, R₁₁, and R₁₇, are independently selected from alkyl, cycloalkyl, alkoxy, OH, oxo, CF₃, and halo, or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O, or wherein A′ may be optionally substituted with 1 or 2 further substituents R₄₃ and R₄₄ independently selected from alkyl, cycloalkyl, alkoxy, OH, oxo, CF₃, and halo, and two different carbon ring members within A′ are connected by a bridging —CH₂-group.
B₁ can be a carbon centre and B₂ can be a carbon centre, wherein B₃ is a single bond between B₂ and the nitrogen atom to which R₁ is attached and B₁ is connected to B₂ via a single bond, or B₁ can be a nitrogen centre and B₂ can be a carbon centre wherein B₁ is connected to B₂ via a double bond; and R₂ can be selected from —C(═O)—N(R₄)(R₅), —C(═O)—O—R₆,—CH₂—NH—C(═O) CH₃,—CH₂—NH—S(═O)₂R₃₅,—CH₂—O—R₁₆,—CH₂—OH, —

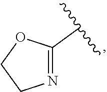 [SMILES: *C1=NCCO1]

CN, and wherein:
R₄ is H or small alkyl, and
R₅ is selected from alkyl, OH, —S(═O)₂-(small alkyl), —(CH₂)₁₋₃—(C═O)—OH, —(CH₂)₀₋₃-cycloalkyl^{b}, —(CH₂)₀₋₃-heterocycloalkyl, —(CH(alkyl))-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, —(C(H)(heteroaryl))-alkyl, —(C(H)(alkyl))—(C═O)—OH, and —(C(H)(heteroaryl))—(C═O)—OR_{28;}
or R₄ and R₅ together with the nitrogen atom to which they are attached can form a carbon-containing 4-, 5-, or 6-membered saturated heterocyclic ring, optionally containing an additional heteroatom selected from N, and O, which may be optionally mono- or di-substituted with substituents selected from oxo, alkyl, alkoxy, OH, halo and CF₃; R₆ is H, small alkyl, and —(CH₂)-phenyl;
R₁₆ is selected from H, small alkyl, —(CH₂)—(C═O)—OH, heterocycloalkyl, and —(CH₂)-heteroaryl; and R₃₅ is selected from H, small alkyl, and cyclopropyl; and

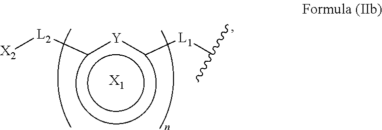

wherein:
n is 1;
L₁ is a linker selected from a single bond and —(CH₂)₁₋₃—;
Y is selected from C, N, O and S; X₁ is phenyl or a 5- or 6-membered carbon-containing aromatic ring containing one, two or three ring members that are selected from N, S, and O;
wherein X₁ may be optionally substituted with 1 or 2 substituents independently selected from alkyl, cycloalkyl, alkoxy, OH, O(cyclopropyl), CF₃, and halo; L₂ is a linker selected from a single bond, —(CH₂)₁₋₃—, and —C(═O)—; and
X₂ is either:
(a) —NR₂OR₂₁, wherein R₂₀ is selected from H, alkyl, and —(CH₂)₀₋₃-R_{22;}
wherein R₂₁ is selected from H, alkyl, and —(CH₂)₀₋₃-R₂₃;

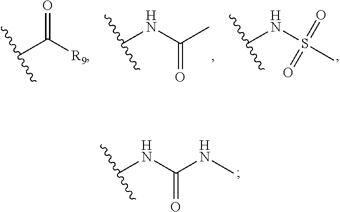

wherein R₂₂ is selected from cycloalkyl, heterocycloalkyl, aryl, heteroaryl,

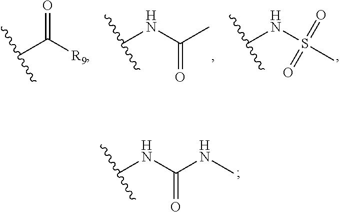

wherein R₂₃ is selected from cycloalkyl, heterocycloalkyl, aryl, heteroaryl, or
(b)₂-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected from alkyl, cycloalkyl, OH, oxo, CF₃, and halo, alkoxy, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O, and X₂ may be further optionally substituted with 1 or 2 substituents R₂₉ and R₃₀, wherein R₂₉ and R₃₀ are independently selected from alkyl, cycloalkyl, alkoxy, OH, oxo, CF₃, and halo; or
(ii) Formula (III) comprising an aromatic ring A fused to a non-aromatic ring A′,

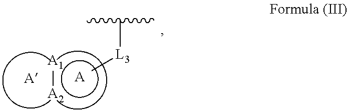

wherein:
L₃ is a single bond;
A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, O(cyclopropyl), CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R₇ is selected from alkyl, alkoxy, and —(CH₂)₀₋₃-R₈,

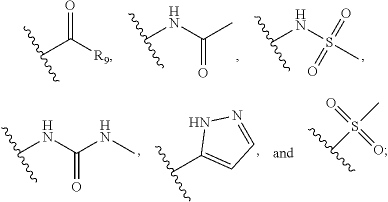

wherein R₈ is selected from cycloalkyl, heterocycloalkyl, wherein R₉ is selected from H, alkyl, OH, N(R₃₁)(R₃₂), and alkoxy;
wherein R₃₁ is H or small alkyl;
wherein R₃₂ is selected from H, small alkyl, and —(CH₂)₀₋₃heteroaryl;
wherein A′ may be optionally substituted with 1, 2, or 3 further substituents R₁₀, R₁₁, and R₁₇, wherein R₁₀, R₁₁, and R₁₇, are independently selected from alkyl, cycloalkyl, alkoxy, OH, oxo, CF₃, and halo, or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O, or wherein A′ may be optionally substituted with 1 or 2 further substituents R₄₃ and R₄₄ independently selected from alkyl, cycloalkyl, alkoxy, OH, oxo, CF₃, and halo, and two different carbon ring members within A′ are connected by a bridging —CH₂-group.
B₄ can be N(R₂), B₅ can be N, and B₆ can be C(R₃), wherein B₄ is connected to B₅ via a single bond and B₅ is connected to B₆ via a double bond, or B₄ can be C(R₂), B₅ can be N, and B₆ can be N(R₃), wherein B₄ is connected to B₅ via a double bond and B₅ is connected to B₆ via a single bond; R₂ can be selected from-C(═O)—N(R₄)(R₅), —C(═O)—O—R₆,—CH₂—NH—C(═O) CH₃,—CH₂—NH—S(═O)₂R₃₅,—CH₂—O—R₁₆,—CH₂—OH, —CN, and

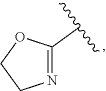

wherein:
R₄ is H or small alkyl, and
R₅ is selected from alkyl, OH, —S(═O)₂-(small alkyl), —(CH₂)₁₋₃—(C═O)—OH, —(CH₂)₀₋₃-cycloalkyl^{b}, —(CH₂)₀₋₃-heterocycloalkyl, —(CH(alkyl)-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, —(C(H)(heteroaryl))-alkyl, —(C(H)(alkyl))—(C═O)—OH, and —(C(H)(heteroaryl))—(C═O)—OR_{28;}
or R₄ and R₅ together with the nitrogen atom to which they are attached can form a carbon-containing 4-, 5-, or 6-membered saturated heterocyclic ring, optionally containing an additional heteroatom selected from N, and O, which may be optionally mono- or di-substituted with substituents selected from oxo, alkyl, alkoxy, OH, halo and CF₃; R₆ is H, small alkyl, and —(CH₂)-phenyl;
R₁₆ is selected from H, small alkyl, —(CH₂)—(C═O)—OH, heterocycloalkyl, and —(CH₂)-heteroaryl; and
R₃₅ is selected from H, small alkyl, and cyclopropyl; and

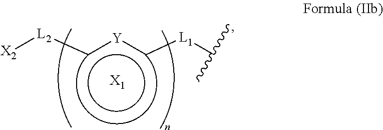

wherein:
n is 1;
L₁ is a linker selected from a single bond and —(CH₂)₁₋₃—;
Y is selected from C, N, O and S; X₁ is phenyl or a 5- or 6-membered carbon-containing aromatic ring containing one, two or three ring members that are selected from N, S, and O;
wherein X₁ may be optionally substituted with 1 or 2 substituents independently selected from alkyl, cycloalkyl, alkoxy, OH, O(cyclopropyl), CF₃, and halo; L₂ is a linker selected from a single bond, —(CH₂)₁₋₃—, and —C(═O)—; and
X₂ is either:
(a) —NR₂OR₂₁, wherein R₂₀ is selected from H, alkyl, and —(CH₂)₀₋₃-R_{22;}
wherein R₂₁ is selected from H, alkyl, and —(CH₂)₀₋₃-R₂₃;

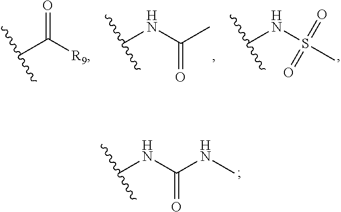

wherein R₂₂ is selected from cycloalkyl, heterocycloalkyl, aryl, heteroaryl,

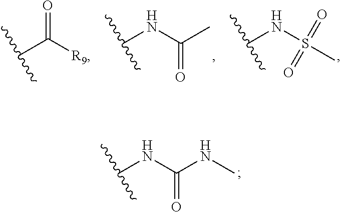

wherein R₂₃ is selected from cycloalkyl, heterocycloalkyl, aryl, heteroaryl, or
(b)₂-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected from alkyl, cycloalkyl, alkoxy, OH, Oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O, and X₂ may be further optionally substituted with 1 or 2 substituents R₂₉ and R₃₀, wherein R₂₉ and R₃₀ are independently selected from alkyl, cycloalkyl, alkoxy, OH, oxo, CF₃, and halo; or
(ii) Formula (III) comprising an aromatic ring A fused to a non-aromatic ring A′,

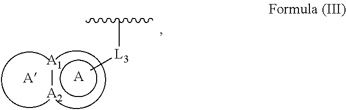

wherein:
L₃ is a single bond;
A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, O(cyclopropyl), CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R₇ is selected from alkyl, alkoxy, and —(CH₂)₀₋₃-R₈,

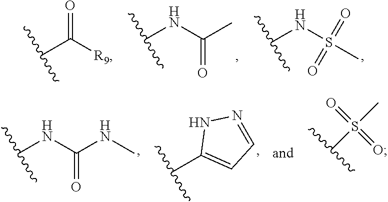

wherein R₈ is selected from cycloalkyl, heterocycloalkyl, wherein R₉ is selected from H, alkyl, OH, N(R₃₁)(R₃₂), and alkoxy;
wherein R₃₁ is H or small alkyl;
wherein R₃₂ is selected from H, small alkyl, and —(CH₂)₀₋₃heteroaryl;
wherein A′ may be optionally substituted with 1, 2, or 3 further substituents R₁₀, R₁₁, and R₁₇, wherein R₁₀, R₁₁, and R₁₇, are independently selected from alkyl, cycloalkyl, alkoxy, OH, oxo, CF₃, and halo, or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O, or wherein A′ may be optionally substituted with 1 or 2 further substituents R₄₃ and R₄₄ independently selected from alkyl, cycloalkyl, alkoxy, OH, oxo, CF₃, and halo, and two different carbon ring members within A′ are connected by a bridging —CH₂-group.
B₁ can be a carbon centre and B₂ can be a carbon centre, wherein B₃ is a single bond between B₂ and the nitrogen atom to which R₁ is attached and B₁ is connected to B₂ via a single bond, or B₁ can be a nitrogen centre and B₂ can be a carbon centre wherein B₁ is connected to B₂ via a double bond; B₄ can be N(R₂), B₅ can be N, and B₆ can be C(R₃), wherein B₄ is connected to B₅ via a single bond and B₅ is connected to B₆ via a double bond, or B₄ can be C(R₂), B₅ can be N, and B₆ can be N(R₃), wherein B₄ is connected to B₅ via a double bond and B₅ is connected to B₆ via a single bond; R₂ can be selected from —C(═O)—N(R₄)(R₅),

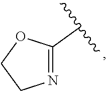 [SMILES: *C1=NCCO1]

—C(═O)—O—R₆,—CH₂—NH—S(═O)₂R₃₅,—CH₂—NH—C(═O) CH₃,—CH₂—O—R₁₆,—CH₂—OH, —CN, and wherein:
R₄ is H or small alkyl, and
R₅ is selected from alkyl, OH, —S(═O)₂-(small alkyl), —(CH₂)₁₋₃—(C═O)—OH, —(CH₂)₀₋₃-cycloalkyl^{b}, —(CH₂)₀₋₃-heterocycloalkyl, —(CH(alkyl))-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, —(C(H)(heteroaryl))-alkyl, —(C(H)(alkyl))—(C═O)—OH, and —(C(H)(heteroaryl))—(C═O)—OR_{28;}
or R₄ and R₅ together with the nitrogen atom to which they are attached can form a carbon-containing 4-, 5-, or 6-membered saturated heterocyclic ring, optionally containing an additional heteroatom selected from N, and O, which may be optionally mono- or di-substituted with substituents selected from oxo, alkyl, alkoxy, OH, halo and CF_{3;}
R₆ is H, small alkyl, and —(CH₂)-phenyl; R₁₆ is selected from H, small alkyl, —(CH₂)—(C═O)—OH, heterocycloalkyl, and —(CH₂)-heteroaryl; and R₃₅ is selected from H, small alkyl, and cyclopropyl; and
R₁ can be:

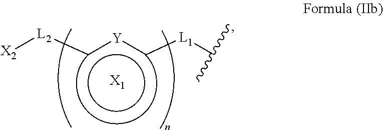

wherein:
n is 1;
L₁ is a linker selected from a single bond and —(CH₂)₁₋₃—;
Y is selected from C, N, O and S; X₁ is phenyl or a 5- or 6-membered carbon-containing aromatic ring containing one, two or three ring members that are selected from N, S, and O;
wherein X₁ may be optionally substituted with 1 or 2 substituents independently selected from alkyl, cycloalkyl, alkoxy, OH, O(cyclopropyl), CF₃, and halo;
L₂ is a linker selected from a single bond, —(CH₂)₁₋₃—, and —C(═O)—; and
X₂ is either:
(a) —NR₂OR₂₁, wherein R₂₀ is selected from H, alkyl, and —(CH₂)₀₋₃-R_{22;}
wherein R₂₁ is selected from H, alkyl, and —(CH₂)₀₋₃-R₂₃;

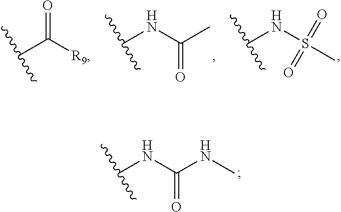

wherein R₂₂ is selected from cycloalkyl, heterocycloalkyl, aryl, heteroaryl,

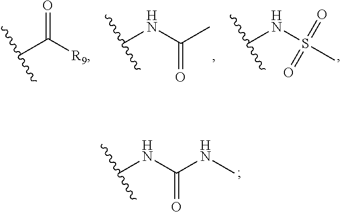

wherein R₂₃ is selected from cycloalkyl, heterocycloalkyl, aryl, heteroaryl, or
(b)₂-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O, and X₂ may be further optionally substituted with 1 or 2 substituents R₂₉ and R₃₀, wherein R₂₉ and R₃₀ are independently selected from alkyl, cycloalkyl, alkoxy, OH, oxo, CF₃, and halo; or
(ii) Formula (III) comprising an aromatic ring A fused to a non-aromatic ring A′,

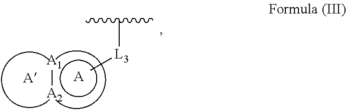

wherein:
L₃ is a single bond;
A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, O(cyclopropyl), CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R₇ is selected from alkyl, alkoxy, and —(CH₂)₀₋₃-R₈,

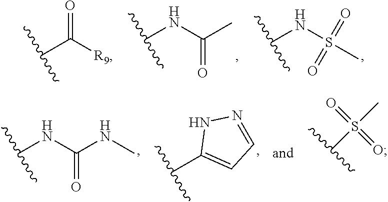

wherein R₈ is selected from cycloalkyl, heterocycloalkyl, wherein R₉ is selected from H, alkyl, OH, N(R₃₁)(R₃₂), and alkoxy;
wherein R₃₁ is H or small alkyl;
wherein R₃₂ is selected from H, small alkyl, and —(CH₂)₀₋₃heteroaryl;
wherein A′ may be optionally substituted with 1, 2, or 3 further substituents R₁₀, R₁₁, and R₁₇, wherein R₁₀, R₁₁, and R₁₇, are independently selected from alkyl, cycloalkyl, alkoxy, OH, oxo, CF₃, and halo, or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O, or wherein A′ may be optionally substituted with 1 or 2 further substituents R₄₃ and R₄₄ independently selected from alkyl, cycloalkyl, alkoxy, OH, oxo, CF₃, and halo, and two different carbon ring members within A′ are connected by a bridging —CH₂-group.
R₂ can be selected from —C(═O)—N(R₄)(R₅), —C(═O)—O—R₆,—CH₂—NH—S(═O)₂R₃₅,—CH₂—NH—C(═O) CH₃,—CH₂—O—R₁₆,

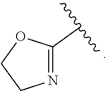 [SMILES: *C1=NCCO1]

—CH₂—OH, —CN, and wherein:
R₄ is H or small alkyl, and
R₅ is selected from alkyl, OH, —S(═O)₂-(small alkyl), —(CH₂)₁₋₃—(C═O)—OH, —(CH₂)₀₋₃-cycloalkyl^{b}, —(CH₂)₀₋₃-heterocycloalkyl, —(CH(alkyl)-heterocycloalkyl, —(CH₂)₀₋₃-polycycloalkyl, —(CH₂)₀₋₃-aryl, —(C(H)(phenyl))-alkyl, —(C(H)(heteroaryl))-alkyl, —(C(H)(alkyl))—(C═O)—OH, and —(C(H)(heteroaryl))—(C═O)—OR_{28;}
or R₄ and R₅ together with the nitrogen atom to which they are attached can form a carbon-containing 4-, 5-, or 6-membered saturated heterocyclic ring, optionally containing an additional heteroatom selected from N, and O, which may be optionally mono- or di-substituted with substituents selected from oxo, alkyl, alkoxy, OH, halo and CF_{3;}
R₆ is H, small alkyl, and —(CH₂)-phenyl;
R₁₆ is selected from H, small alkyl, —(CH₂)—(C═O)—OH, heterocycloalkyl, and —(CH₂)-heteroaryl; and R₃₅ is selected from H, small alkyl, and cyclopropyl; and
R₁ can be:
(i) Formula (IIb),

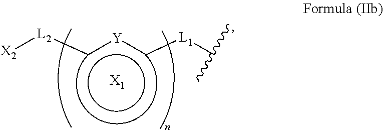

wherein:
n is 1;
L₁ is a linker selected from a single bond and —(CH₂)₁₋₃—; Y is selected from C, N, O and S;
X₁ is phenyl or a 5- or 6-membered carbon-containing aromatic ring containing one, two or three ring members that are selected from N, S, and O;
wherein X₁ may be optionally substituted with 1 or 2 substituents independently selected from alkyl, cycloalkyl, alkoxy, OH, O(cyclopropyl), CF₃, and halo; L₂ is a linker selected from a single bond, —(CH₂)₁₋₃—, and —C(═O)—; and X₂ is either:
(a) —NR₂OR₂₁, wherein R₂₀ is selected from H, alkyl, and —(CH₂)₀₋₃-R_{22;}
wherein R₂₁ is selected from H, alkyl, and —(CH₂)₀₋₃-R₂₃;

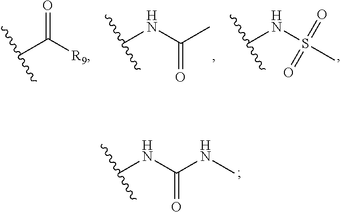

wherein R₂₂ is selected from cycloalkyl, heterocycloalkyl, aryl, heteroaryl,

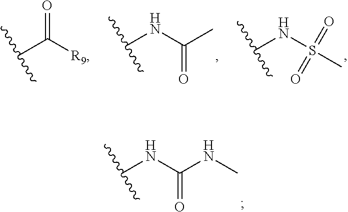

wherein R₂₃ is selected from cycloalkyl, heterocycloalkyl, aryl, heteroaryl, or
(b)₂-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected from alkyl, cycloalkyl, OH, alkoxy, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O, and X₂ may be further optionally substituted with 1 or 2 substituents R₂₉ and R₃₀, wherein R₂₉ and R₃₀ are independently selected from alkyl, cycloalkyl, alkoxy, OH, oxo, CF₃, and halo; or

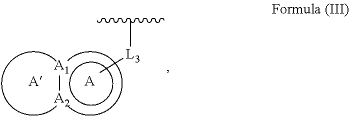

wherein:
L₃ is a single bond;
A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, O(cyclopropyl), CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R₇ is selected from alkyl, alkoxy, and —(CH₂)₀₋₃-R₈, wherein R₈ is selected from cycloalkyl, heterocycloalkyl,

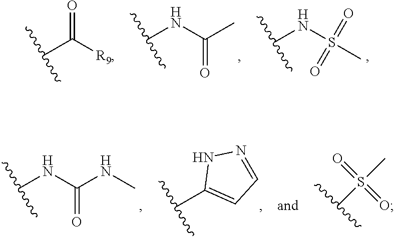

wherein R₉ is selected from H, alkyl, OH, N(R₃₁)(R₃₂), and alkoxy;
wherein R₃₁ is H or small alkyl;
wherein R₃₂ is selected from H, small alkyl, and —(CH₂)₀₋₃heteroaryl;
wherein A’ may be optionally substituted with 1, 2, or 3 further substituents R₁₀, R₁₁, and R₁₇, wherein R₁₀, R₁₁, and R₁₇, are independently selected from alkyl, cycloalkyl, alkoxy, OH, oxo, CF₃, and halo, or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O, or wherein A′ may be optionally substituted with 1 or 2 further substituents R₄₃ and R₄₄ independently selected from alkyl, cycloalkyl, alkoxy, OH, oxo, CF₃, and halo, and two different carbon ring members within A′ are connected by a bridging —CH₂-group.
B₁ can be a carbon centre and B₂ can be a carbon centre, wherein B₃ is a single bond between B₂ and the nitrogen atom to which R₁ is attached and B₁ is connected to B₂ via a single bond, or B₁ can be a nitrogen centre and B₂ can be a carbon centre wherein B₁ is connected to B₂ via a double bond; and R₁ can be:
(i) Formula (IIb),

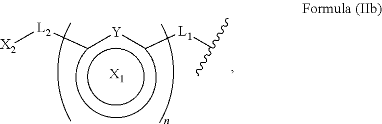

wherein:
n is 1;
L₁ is a linker selected from a single bond, —(CH₂)₁₋₃—, —(CH)(alkyl)-, and —C(alkyl)(alkyl)-; Y is selected from C, N, O and S;
X₁ is phenyl or a 5- or 6-membered carbon-containing aromatic ring containing one, two or three ring members that are selected from N, S, and O;
wherein X₁ may be optionally substituted with 1 or 2 substituents independently selected from alkyl, cycloalkyl, alkoxy, OH, CF₃, and halo;
L₂ is a linker selected from a single bond, —(CH₂)₁₋₃—, —(CH)(alkyl)-, —C(alkyl)(alkyl)-, —C(═O)—, and —SO₂—; and
X₂ is either:
(a) —NR₂OR₂₁, wherein R₂₀ is selected from H, alkyl, and —(CH₂)₀₋₃-R_{22;}
wherein R₂₁ is selected from H, alkyl, and —(CH₂)₀₋₃-R₂₃;

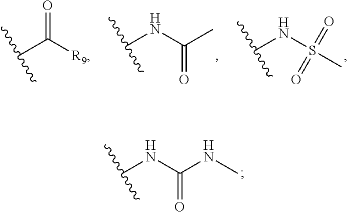

wherein R₂₂ is selected from cycloalkyl, heterocycloalkyl, aryl, heteroaryl,

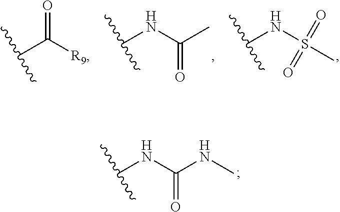

wherein R₂₃ is selected from cycloalkyl, heterocycloalkyl, aryl, heteroaryl, or
(b)₂-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O, and X₂ may be further optionally substituted with 1 or 2 substituents R₂₉ and R₃₀, wherein R₂₉ and R₃₀ are independently selected from alkyl, cycloalkyl, alkoxy, OH, oxo, CF₃, and halo; or
(ii) Formula (III) comprising an aromatic ring A fused to a non-aromatic ring A′,

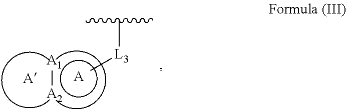

wherein:
L₃ is a single bond;
A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R, is selected from alkyl, alkoxy, and —(CH₂)₀₋₃-R₈,

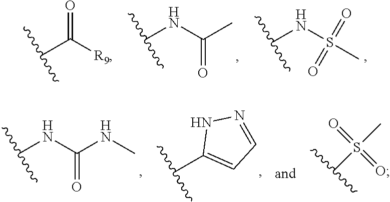

wherein R₈ is selected from cycloalkyl, heterocycloalkyl, wherein R₉ is selected from H, alkyl, OH, N(R₃₁)(R₃₂), and alkoxy;
wherein R₃₁ is H or small alkyl;
wherein R₃₂ is selected from H, small alkyl, and —(CH₂)₀₋₃heteroaryl;
wherein A′ may be optionally substituted with 1, 2, or 3 further substituents R₁₀, R₁₁, and R₁₇, wherein R₁₀, R₁₁, and R₁₇, are independently selected from alkyl, cycloalkyl, alkoxy, OH, oxo, CF₃, and halo, or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O.
B₁ can be a carbon centre and B₂ can be a carbon centre, wherein B₃ is a single bond between B₂ and the nitrogen atom to which R₁ is attached and B₁ is connected to B₂ via a single bond, or B₁ can be a nitrogen centre and B₂ can be a carbon centre wherein B₁ is connected to B₂ via a double bond; and
R₁ can be:
(i) Formula (IIb),

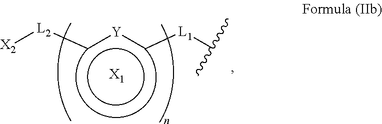

wherein:
n is 1;
L₁ is a linker selected from a single bond, —(CH₂)₁₋₃—, —(CH)(alkyl)-, and —C(alkyl)(alkyl)-; Y is selected from C, N, O and S;
X₁ is phenyl or a 5- or 6-membered carbon-containing aromatic ring containing one, two or three
wherein X₁ may be optionally substituted with 1 or 2 substituents independently selected from alkyl, cycloalkyl, alkoxy, OH, CF₃, and halo; L₂ is a linker selected from a single bond, —(CH₂)₁₋₃—, —(CH)(alkyl)-, —C(alkyl)(alkyl)-, —C(═O)—, and —SO₂—; and
X₂ is either:
(a) —NR₂OR₂₁, wherein R₂₀ is selected from H, alkyl, and —(CH₂)₀₋₃-R_{22;}
wherein R₂₁ is selected from H, alkyl, and —(CH₂)₀₋₃-R₂₃;

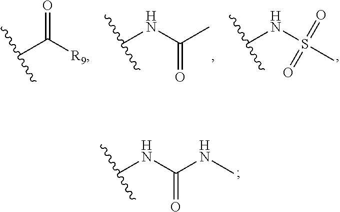

wherein R₂₂ is selected from cycloalkyl, heterocycloalkyl, aryl, heteroaryl,

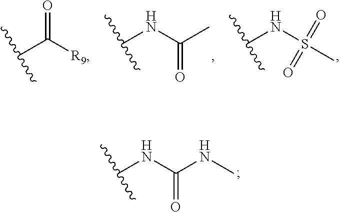

wherein R₂₃ is selected from cycloalkyl, heterocycloalkyl, aryl, heteroaryl, or
(b)₂-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O; or
(ii) Formula (III) comprising an aromatic ring A fused to a non-aromatic ring A′,

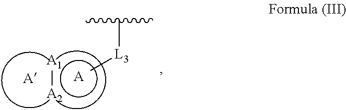

wherein:
L₃ is a single bond;
A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R₇ is selected from alkyl, alkoxy, and —(CH₂)₀₋₃-R₈,

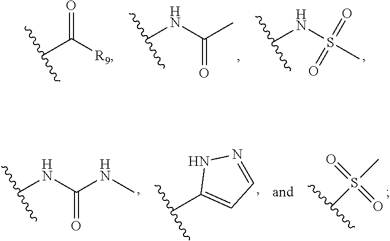

wherein R₈ is selected from cycloalkyl, heterocycloalkyl, wherein R₉ is selected from H, alkyl, OH, NH₂, and alkoxy;
wherein A′ may be optionally substituted with 1, 2, or 3 further substituents R₁₀, R₁₁, and R₁₇, wherein R₁₀, R₁₁, and R₁₇, are independently selected from alkyl, cycloalkyl, alkoxy, OH, oxo, CF₃, and halo, or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O.
B₁ can be a carbon centre and B₂ can be a carbon centre, wherein B₃ is a single bond between B₂ and the nitrogen atom to which R₁ is attached and B₁ is connected to B₂ via a single bond; and
R₁ can be:
(i) Formula (IIa),

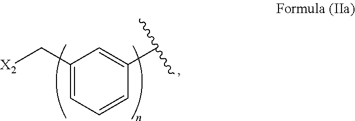

wherein:
n is 1;
X₂ is a non-aromatic, carbon-containing mono- or bi-cyclic ring containing 5, 6, or 7 ring members, wherein at least one ring member is independently selected from N, S, and O;
wherein X₂ may be optionally substituted with 1 or 2 substituents R₁₈ and R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O; or
(ii) Formula (III) comprising an aromatic ring A fused to a non-aromatic ring A′,

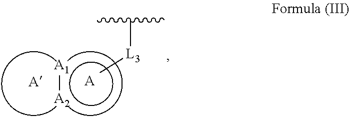

wherein:
L₃ is a single bond;
A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R₇ is selected from alkyl, alkoxy, and —(CH₂)₀₋₃-R₈,

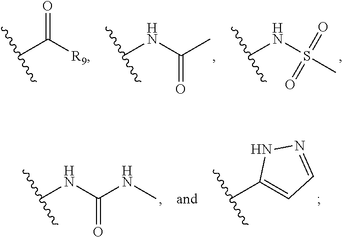

wherein R₈ is selected from cycloalkyl, heterocycloalkyl, wherein R₉ is selected from H, alkyl, OH, NH₂, and alkoxy;
wherein A′ may be optionally substituted with 1 or 2 further substituents R₁₀ and R₁₁, wherein R₁₀ and R₁₁ are independently selected from alkyl, cycloalkyl, alkoxy, OH, oxo, CF₃, and halo, or wherein A′ may be optionally substituted with 1 further substituent R₂₄ selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O.
B₄ can be N(R₂), B₅ can be N, and B₆ can be C(R₃), wherein B₄ is connected to B₅ via a single bond and B₅ is connected to B₆ via a double bond, or B₄ can be C(R₂), Bs can be N, and B₆ can be N(R₃), wherein B₄ is connected to B₅ via a double bond and B₅ is connected to B₆ via a single bond; and R₁ can be:
(i) Formula (IIb),

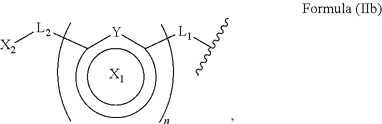

wherein:
n is 1;
L₁ is a linker selected from a single bond, —(CH₂)₁₋₃—, —(CH)(alkyl)-, and —C(alkyl)(alkyl)-; Y is selected from C, N, O and S;
X₁ is phenyl or a 5- or 6-membered carbon-containing aromatic ring containing one, two or three ring members that are selected from N, S, and O;
wherein X₁ may be optionally substituted with 1 or 2 substituents independently selected from alkyl, cycloalkyl, alkoxy, OH, CF₃, and halo;
L₂ is a linker selected from a single bond, —(CH₂)₁₋₃—, —(CH)(alkyl)-, —C(alkyl)(alkyl)-, —C(═O)—, and —SO₂—; and
X₂ is either:
(a) —NR₂OR₂₁, wherein R₂₀ is selected from H, alkyl, and —(CH₂)₀₋₃-R_{22;}
wherein R₂₁ is selected from H, alkyl, and —(CH₂)₀₋₃-R₂₃;

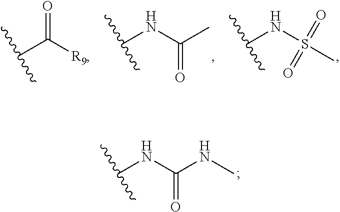

wherein R₂₂ is selected from cycloalkyl, heterocycloalkyl, aryl, heteroaryl,

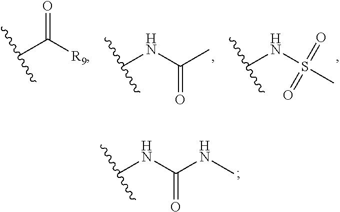

wherein R₂₃ is selected from cycloalkyl, heterocycloalkyl, aryl, heteroaryl, or
(b)₂-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O, and X₂ may be further optionally substituted with 1 or 2 substituents R₂₉ and R₃₀, wherein R₂₉ and R₃₀ are independently selected from alkyl, cycloalkyl, alkoxy, OH, oxo, CF₃, and halo; or
(ii) Formula (III) comprising an aromatic ring A fused to a non-aromatic ring A′,

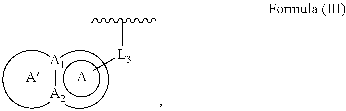

wherein:
L₃ is a single bond;
A₁ and A₂ are both carbon;
A is phenyl or a 5- or 6-membered carbon-containing aromatic ring containing one, two or three
wherein A may be optionally substituted with 1 or 2 substituents independently selected from alkyl, cycloalkyl, alkoxy, OH, CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R₇ is selected from alkyl, alkoxy, and
—(CH₂)₀₋₃-R₈,
wherein R₈ is selected from cycloalkyl, heterocycloalkyl,

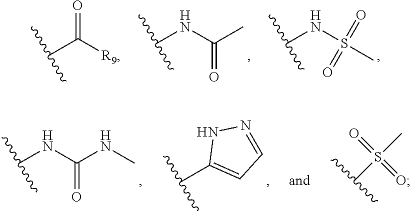

wherein R₉ is selected from H, alkyl, OH, N(R₃₁)(R₃₂), and alkoxy;
wherein R₃₁ is H or small alkyl;
wherein R₃₂ is selected from H, small alkyl, and —(CH₂)₀₋₃heteroaryl;
wherein A′ may be optionally substituted with 1, 2, or 3 further substituents R₁₀, R₁₁, and R₁₇, wherein R₁₀, R₁₁, and R₁₇, are independently selected from alkyl, cycloalkyl, alkoxy, OH, oxo, CF₃, and halo, or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O.
B₄ can be N(R₂), B₅ can be N, and B₆ can be C(R₃), wherein B₄ is connected to B₅ via a single bond and B₅ is connected to B₆ via a double bond, or B₄ can be C(R₂), B₅ can be N, and B₆ can be N(R₃), wherein B₄ is connected to B₅ via a double bond and B₅ is connected to B₆ via a single bond; and
R₁ can be:
(i) Formula (IIb),

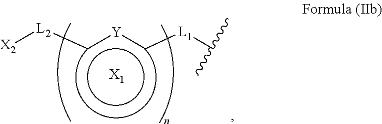

wherein:
n is 1;
L₁ is a linker selected from a single bond, —(CH₂)₁₋₃—, —(CH)(alkyl)-, and —C(alkyl)(alkyl)-; Y is selected from C, N, O and S;
X₁ is phenyl or a 5- or 6-membered carbon-containing aromatic ring containing one, two or three ring members that are selected from N, S, and O;
wherein X₁ may be optionally substituted with 1 or 2 substituents independently selected from alkyl, cycloalkyl, alkoxy, OH, CF₃, and halo;
L₂ is a linker selected from a single bond, —(CH₂)₁₋₃—, —(CH)(alkyl)-, —C(alkyl)(alkyl)-, —C(═O)—, and —SO₂—; and either:
wherein R₂₀ is selected from H, alkyl, and —(CH₂)₀₋₃-R_{22;}
wherein R₂₁ is selected from H, alkyl, and —(CH₂)₀₋₃-R_{23;}
X₂ is (a) —NR₂OR₂₁,

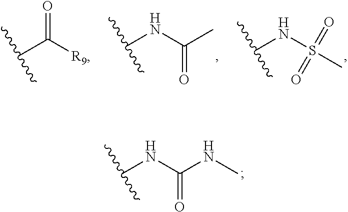

wherein R₂₂ is selected from cycloalkyl, heterocycloalkyl, aryl, heteroaryl,

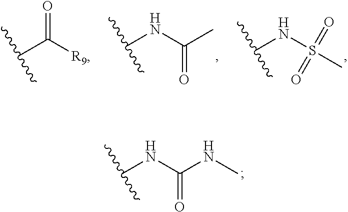

wherein R₂₃ is selected from cycloalkyl, heterocycloalkyl, aryl, heteroaryl, or
(b)₂-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O; or

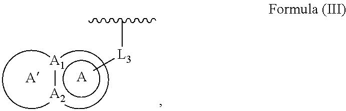

wherein:
L₃ is a single bond;
A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R, is selected from alkyl, alkoxy, and —(CH₂)₀₋₃-R₈,

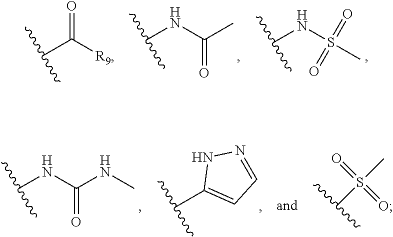

wherein R₈ is selected from cycloalkyl, heterocycloalkyl, wherein R₉ is selected from H, alkyl, OH, NH₂, and alkoxy;
wherein A′ may be optionally substituted with 1, 2, or 3 further substituents R₁₀, R₁₁, and R₁₇, wherein R₁₀, R₁₁, and R₁₇, are independently selected from alkyl, cycloalkyl, alkoxy, OH, oxo, CF₃, and halo, or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O.
B₄ can be C(R₂), Bs can be N, and B₆ can be N(R₃), wherein B₄ is connected to Bs via a double bond and Bs is connected to B₆ via a single bond; and
R₁ can be:
(i) Formula (IIa),

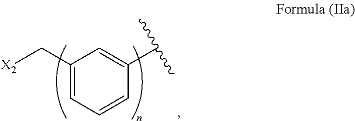

wherein:
n is 1;
X₂ is a non-aromatic, carbon-containing mono- or bi-cyclic ring containing 5, 6, or 7 ring members, wherein at least one ring member is independently selected from N, S, and O;
wherein X₂ may be optionally substituted with 1 or 2 substituents R₁₈ and R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O; or
(ii) Formula (III) comprising an aromatic ring A fused to a non-aromatic ring A′,

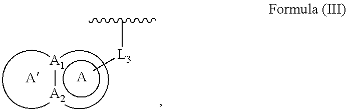

wherein:
L₃ is a single bond;
A₁ and Az are both carbon;
A is phenyl or a 5- or 6-membered carbon-containing aromatic ring containing one, two or three
wherein A may be optionally substituted with 1 or 2 substituents independently selected from alkyl, cycloalkyl, alkoxy, OH, CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R₇ is selected from alkyl, alkoxy, and —(CH₂)₀₋₃-R₈,

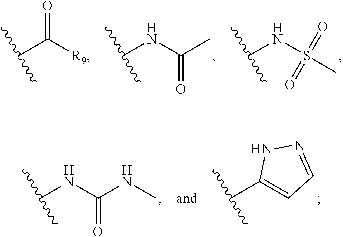

wherein R₈ is selected from cycloalkyl, heterocycloalkyl, wherein R₉ is selected from H, alkyl, OH, NH₂, and alkoxy;
wherein A′ may be optionally substituted with 1 or 2 further substituents R₁₀ and R₁₁, wherein R₁₀ and R₁₁ are independently selected from alkyl, cycloalkyl, alkoxy, OH, oxo, CF₃, and halo, or wherein A′ may be optionally substituted with 1 further substituent R₂₄ selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O.
B₁ can be a carbon centre and B₂ can be a carbon centre, wherein B₃ is a single bond between B₂ and the nitrogen atom to which R₁ is attached and B₁ is connected to B₂ via a single bond, or B₁ can be a nitrogen centre and B₂ can be a carbon centre wherein B₁ is connected to B₂ via a double bond; and B₄ can be N(R₂), B₅ can be N, and B₆ can be C(R₃), wherein B₄ is connected to B₅ via a single bond and B₅ is connected to B₆ via a double bond, or B₄ can be C(R₂), B₅ can be N, and B₆ can be N(R₃), wherein B₄ is connected to B₅ via a double bond and B₅ is connected to B₆ via a single bond; and
R₁ can be:
(i) Formula (IIb),

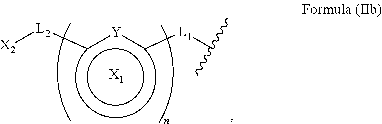

wherein:
n is 1;
L₁ is a linker selected from a single bond, —(CH₂)₁₋₃—, —(CH)(alkyl)-, and —C(alkyl)(alkyl)-; Y is selected from C, N, O and S;
X₁ is phenyl or a 5- or 6-membered carbon-containing aromatic ring containing one, two or three ring members that are selected from N, S, and O;
wherein X₁ may be optionally substituted with 1 or 2 substituents independently selected from alkyl, cycloalkyl, alkoxy, OH, CF₃, and halo;
L₂ is a linker selected from a single bond, —(CH₂)₁₋₃—, —(CH)(alkyl)-, —C(alkyl)(alkyl)-, —C(═O)—, and —SO₂—; and either:
wherein R₂₀ is selected from H, alkyl, and —(CH₂)₀₋₃-R_{2;}
wherein R₂₁ is selected from H, alkyl, and —(CH₂)₀₋₃-R_{23;}
X₂ is (a) —NR₂OR₂₁,

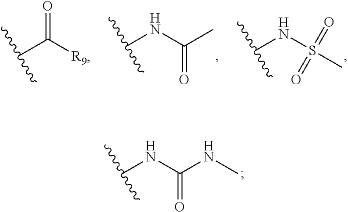

wherein R₂₂ is selected from cycloalkyl, heterocycloalkyl, aryl, heteroaryl,

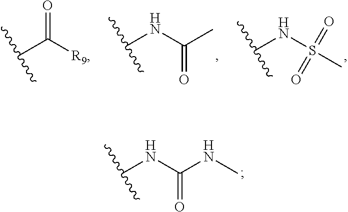

wherein R₂₃ is selected from cycloalkyl, heterocycloalkyl, aryl, heteroaryl, or
(b)₂-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected from alkyl, cycloalkyl, alkoxy, OH, CF₃, and oxo, halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O, and X₂ may be further optionally substituted with 1 or 2 substituents R₂₉ and R₃₀, wherein R₂₉ and R₃₀ are independently selected from alkyl, cycloalkyl, alkoxy, OH, oxo, CF₃, and halo; or

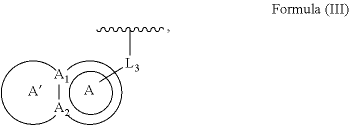

wherein:
L₃ is a single bond;
A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R₇ is selected from alkyl, alkoxy, and —(CH₂)₀₋₃-R₈,

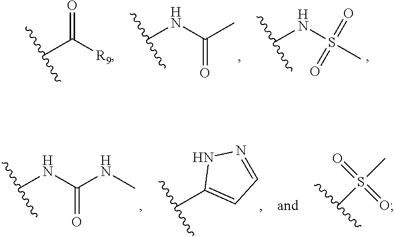

wherein R₈ is selected from cycloalkyl, heterocycloalkyl, wherein R₉ is selected from H, alkyl, OH, N(R₃₁)(R₃₂), and alkoxy;
wherein R₃₁ is H or small alkyl;
wherein R₃₂ is selected from H, small alkyl, and —(CH₂)₀₋₃heteroaryl;
wherein A′ may be optionally substituted with 1, 2, or 3 further substituents R₁₀, R₁₁, and R₁₇, wherein R₁₀, R₁₁, and R₁₇, are independently selected from alkyl, cycloalkyl, alkoxy, OH, oxo, CF₃, and halo, or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O.
B₁ can be a carbon centre and B₂ can be a carbon centre, wherein B₃ is a single bond between B₂ and the nitrogen atom to which R₁ is attached and B₁ is connected to B₂ via a single bond, or B₁ can be a nitrogen centre and B₂ can be a carbon centre wherein B₁ is connected to B₂ via a double bond; and B₄ can be N(R₂), B₅ can be N, and B₆ can be C(R₃), wherein B₄ is connected to B₅ via a single bond and B₅ is connected to B₆ via a double bond, or B₄ can be C(R₂), Bs can be N, and B₆ can be N(R₃), wherein B₄ is connected to B₅ via a double bond and B₅ is connected to B₆ via a single bond; and
R₁ can be:
(i) Formula (IIb),

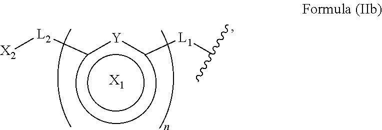

wherein:
n is 1;
L₁ is a linker selected from a single bond, —(CH₂)₁₋₃—, —(CH)(alkyl)-, and —C(alkyl)(alkyl);
Y is selected from C, N, O and S;
X₁ is phenyl or a 5- or 6-membered carbon-containing aromatic ring containing one, two or three ring members that are selected from N, S, and O;
wherein X₁ may be optionally substituted with 1 or 2 substituents independently selected from alkyl, cycloalkyl, alkoxy, OH, CF₃, and halo;
L₂ is a linker selected from a single bond, —(CH₂)₁₋₃—, —(CH)(alkyl)-, —C(alkyl)(alkyl)-, —C(═O)—, and —SO₂—; and
X₂ is either:
(a) —NR₂OR₂₁, wherein R₂₀ is selected from H, alkyl, and —(CH₂)₀₋₃-R_{22;}
wherein R₂₁ is selected from H, alkyl, and —(CH₂)₀₋₃-R₂₃;

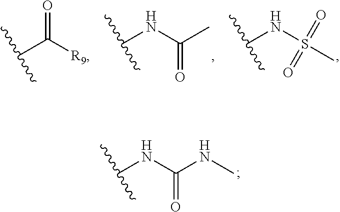

wherein R₂₂ is selected from cycloalkyl, heterocycloalkyl, aryl, heteroaryl,

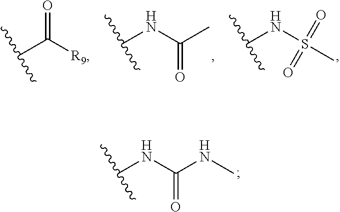

wherein R₂₃ is selected from cycloalkyl, heterocycloalkyl, aryl, heteroaryl, or
(b)₂-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected from alkyl, cycloalkyl, alkoxy, OH, Oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O; or
(ii) Formula (III) comprising an aromatic ring A fused to a non-aromatic ring A′,

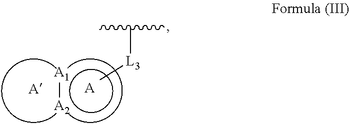

wherein:
L₃ is a single bond;
A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R₇ is selected from alkyl, alkoxy, and —(CH₂)₀₋₃-R₈,

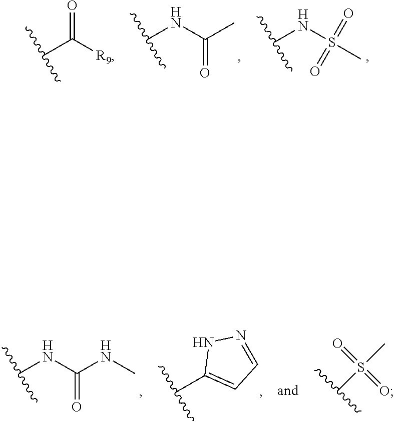

wherein R₈ is selected from cycloalkyl, heterocycloalkyl, wherein R₉ is selected from H, alkyl, OH, NH₂, and alkoxy;
wherein A′ may be optionally substituted with 1, 2, or 3 further substituents R₁₀, R₁₁, and R₁₇, wherein R₁₀, R₁₁, and R₁₇, are independently selected from alkyl, cycloalkyl, alkoxy, OH, oxo, CF₃, and halo, or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O.
B₁ can be a carbon centre and B₂ can be a carbon centre, wherein B₃ is a single bond between B₂ and the nitrogen atom to which R₁ is attached and B₁ is connected to B₂ via a single bond, and B₄ can be C(R₂), B₅ can be N, and B₆ can be N(R₃), wherein B₄ is connected to B₅ via a double bond and B₅ is connected to B₆ via a single bond; and
R₁ can be:
(i) Formula (IIa),

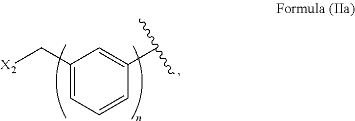

wherein:
n is 1;
X₂ is a non-aromatic, carbon-containing mono- or bi-cyclic ring containing 5, 6, or 7 ring members, wherein at least one ring member is independently selected from N, S, and O;
wherein X₂ may be optionally substituted with 1 or 2 substituents R₁₈ and R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O; or

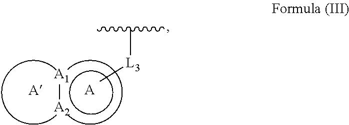

wherein:
L₃ is a single bond;
A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R₇ is selected from alkyl, alkoxy, and —(CH₂)₀₋₃-R₃,

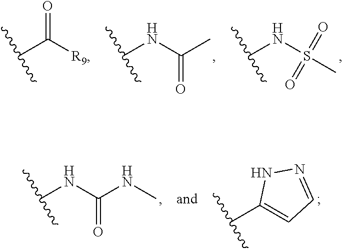

wherein R₈ is selected from cycloalkyl, heterocycloalkyl, wherein R₉ is selected from H, alkyl, OH, NH₂, and alkoxy;
wherein A′ may be optionally substituted with 1 or 2 further substituents R₁₀ and R₁₁, wherein R₁₀ and Ru are independently selected from alkyl, cycloalkyl, alkoxy, OH, oxo, CF₃, and halo, or wherein A′ may be optionally substituted with 1 further substituent R₂₄ selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O.
B₁ can be a carbon centre and B₂ can be a carbon centre, wherein B₃ is a single bond between B₂ and the nitrogen atom to which R₁ is attached and B₁ is connected to B₂ via a single bond, or B₁ can be a nitrogen centre and B₂ can be a carbon centre wherein B₁ is connected to B₂ via a double bond; R₂ can be selected from —C(═O)—N(R₄)(R₅), —C(═O)—O—R₆,—CH₂—NH—C(═O) CH₃,—CH₂—NH—S(═O) z (cyclopropyl),—CH₂—O—R₁₆,—CH₂—OH,

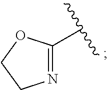 [SMILES: *C1=NCCO1]

—CN, and and
R₁ can be:
(i) Formula (IIb),

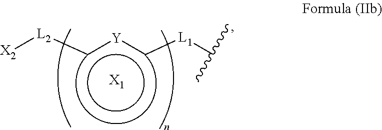

wherein:
n is 1;
L₁ is a linker selected from a single bond, —(CHz)₁₋₃—, —(CH)(alkyl)-, and —C(alkyl)(alkyl)-; Y is selected from C, N, O and S;
X₁ is phenyl or a 5- or 6-membered carbon-containing aromatic ring containing one, two or three ring members that are selected from N, S, and O;
wherein X₁ may be optionally substituted with 1 or 2 substituents independently selected from alkyl, cycloalkyl, alkoxy, OH, CF₃, and halo;
L₂ is a linker selected from a single bond, —(CH₂)₁₋₃—, —(CH)(alkyl)-, —C(alkyl)(alkyl)-, —C(═O)—, and —SO₂—; and
X₂ is either: (a) —NR₂OR₂₁, wherein R₂₀ is selected from H, alkyl, and —(CH₂)₀₋₃-R_{22;}
wherein R₂₁ is selected from H, alkyl, and —(CH₂)₀₋₃-R₂₃;

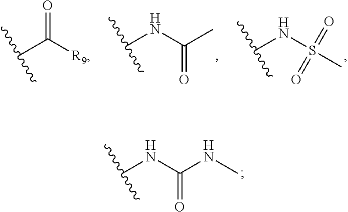

wherein R₂₂ is selected from cycloalkyl, heterocycloalkyl, aryl, heteroaryl,

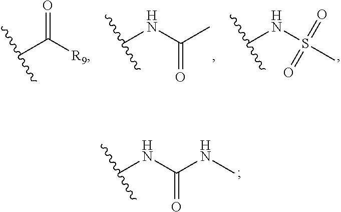

wherein R₂₃ is selected from cycloalkyl, heterocycloalkyl, aryl, heteroaryl, or
(b)₂-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O, and X₂ may be further optionally substituted with 1 or 2 substituents R₂₉ and R₃₀, wherein R₂₉ and R₃₀ are independently selected from alkyl, cycloalkyl, alkoxy, OH, oxo, CF₃, and halo; or
(ii) Formula (III) comprising an aromatic ring A fused to a non-aromatic ring A′,

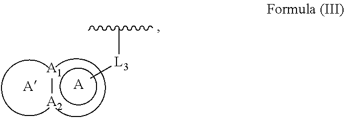

wherein:
L₃ is a single bond;
A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R, is selected from alkyl, alkoxy, and —(CH₂)₀₋₃-R₈,

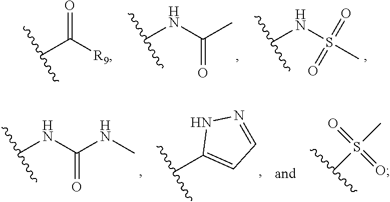

wherein R₈ is selected from cycloalkyl, heterocycloalkyl, wherein R₉ is selected from H, alkyl, OH, N(R₃₁)(R₃₂), and alkoxy;
wherein R₃₁ is H or small alkyl;
wherein R₃₂ is selected from H, small alkyl, and —(CH₂)₀₋₃heteroaryl;
wherein A′ may be optionally substituted with 1, 2, or 3 further substituents Rio, R₁₁, and R₁₇, wherein R₁₀, R₁₁, and R₁₇, are independently selected from alkyl, cycloalkyl, alkoxy, OH, oxo, CF₃, and halo, or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O.
B₁ can be a carbon centre and B₂ can be a carbon centre, wherein B₃ is a single bond between B₂ and the nitrogen atom to which R₁ is attached and B₁ is connected to B₂ via a single bond, or Bi can be a nitrogen centre and B₂ can be a carbon centre wherein B₁ is connected to B₂ via a double bond; R₂ can be selected from —C(═O)—N(R₄)(R₅), —C(═O)—O—R₆,—CH₂—NH—S(═O)₂(cyclopropyl),—CH₂—O—CH₃,—CH₂—OH, and —CN; and R₁ can be:
(i) Formula (IIb),

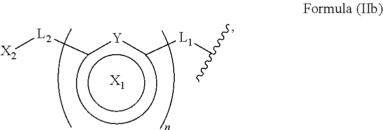

wherein:
n is 1;
L₁ is a linker selected from a single bond, —(CH₂)₁₋₃—, —(CH)(alkyl)-, and —C(alkyl)(alkyl); Y is selected from C, N, O and S;
X₁ is phenyl or a 5- or 6-membered carbon-containing aromatic ring containing one, two or three ring members that are selected from N, S, and O;
wherein X₁ may be optionally substituted with 1 or 2 substituents independently selected from alkyl, cycloalkyl, alkoxy, OH, CF₃, and halo;
L₂ is a linker selected from a single bond, —(CH₂)₁₋₃—, —(CH)(alkyl)-, —C(alkyl)(alkyl)-, —C(═O)—, and —SO₂—; and either:
wherein R₂₀ is selected from H, alkyl, and —(CH₂)₀₋₃-R_{22;}
wherein R₂₁ is selected from H, alkyl, and —(CH₂)₀₋₃-R₂₃;

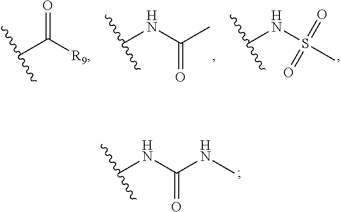

X₂ is (a) —NR₂OR₂₁, wherein R₂₂ is selected from cycloalkyl, heterocycloalkyl, aryl, heteroaryl,

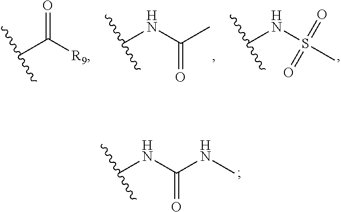

wherein R₂₃ is selected from cycloalkyl, heterocycloalkyl, aryl, heteroaryl, or
(b)₂-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O; or

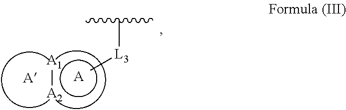

wherein:
L₃ is a single bond;
A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R₇ is selected from alkyl, alkoxy, and —(CH₂)₀₋₃-R₈,

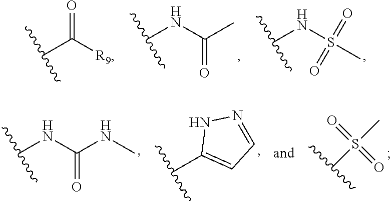

wherein R₈ is selected from cycloalkyl, heterocycloalkyl, wherein R₉ is selected from H, alkyl, OH, NH₂, and alkoxy;
wherein A′ may be optionally substituted with 1, 2, or 3 further substituents R₁₀, R₁₁, and R₁₇, wherein R₁₀, R₁₁, and R₁₇, are independently selected from alkyl, cycloalkyl, alkoxy, OH, oxo, CF₃, and halo, or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O.
B₁ can be a carbon centre and B₂ can be a carbon centre, wherein B₃ is a single bond between B₂ and the nitrogen atom to which R₁ is attached and B₁ is connected to B₂ via a single bond; R₂ can be selected from —C(═O)—N(R₄)(R₅), —C(═O)—O—R₆,—CH₂—O—CH₃, and —CN; and

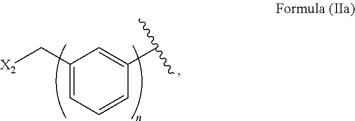

wherein:
n is 1;
X₂ is a non-aromatic, carbon-containing mono- or bi-cyclic ring containing 5, 6, or 7 ring members, wherein at least one ring member is independently selected from N, S, and O;
wherein X₂ may be optionally substituted with 1 or 2 substituents R₁₈ and R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O; or
(ii) Formula (III) comprising an aromatic ring A fused to a non-aromatic ring A′,

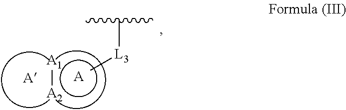

wherein:
L₃ is a single bond;
A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R, is selected from alkyl, alkoxy, and —(CH₂)₀₋₃-R₈,

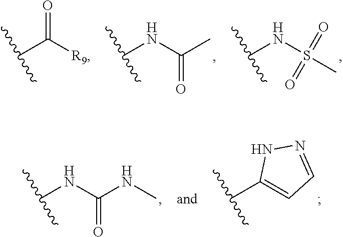

wherein R₈ is selected from cycloalkyl, heterocycloalkyl, wherein R₉ is selected from H, alkyl, OH, NH₂, and alkoxy;
wherein A′ may be optionally substituted with 1 or 2 further substituents R₁₀ and R₁₁, wherein R₁₀ and R₁₁ are independently selected from alkyl, cycloalkyl, alkoxy, OH, oxo, CF₃, and halo, or wherein A′ may be optionally substituted with 1 further substituent R₂₄ selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O.
B₄ can be N(R₂), B₅ can be N, and B₆ can be C(R₃), wherein B₄ is connected to B₅ via a single bond and B₅ is connected to B₆ via a double bond, or B₄ can be C(R₂), B₅ can be N, and B₆ can be N(R₃), wherein B₄ is connected to B₅ via a double bond and B₅ is connected to B₆ via a single bond; R₂ can be selected from —C(═O)—N(R₄)(R₅), —C(═O)—O—R₆,—CH₂—NH—C(═O) CH₃,—CH₂—NH—S(═O)₂(cyclopropyl),—CH₂—O—R₁₆,—CH₂—OH, —CN,

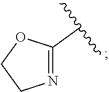 [SMILES: *C1=NCCO1]

and and
R₁ can be:
(i) Formula (IIb),

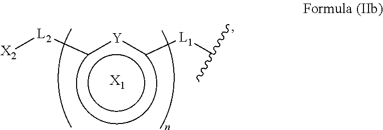

wherein:
n is 1; L₁ is a linker selected from a single bond, —(CH₂)₁₋₃—, —(CH)(alkyl)-, and —C(alkyl)(alkyl); Y is selected from C, N, O and S;
X₁ is phenyl or a 5- or 6-membered carbon-containing aromatic ring containing one, two or three ring members that are selected from N, S, and O;
wherein X₁ may be optionally substituted with 1 or 2 substituents independently selected from alkyl, cycloalkyl, alkoxy, OH, CF₃, and halo;
L₂ is a linker selected from a single bond, —(CH₂)₁₋₃—, —(CH)(alkyl)-, —C(alkyl)(alkyl)-, —C(═O)—, and —SO₂—; and either:
wherein R₂₀ is selected from H, alkyl, and —(CH₂)₀₋₃-R_{22;}
wherein R₂₁ is selected from H, alkyl, and —(CH₂)₀₋₃-R_{23;}
X₂ is (a) —NR₂OR₂₁,

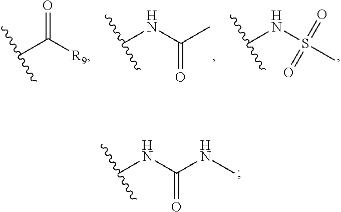

wherein R₂₂ is selected from cycloalkyl, heterocycloalkyl, aryl, heteroaryl,

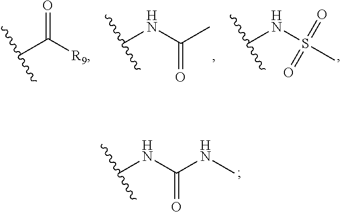

wherein R₂₃ is selected from cycloalkyl, heterocycloalkyl, aryl, heteroaryl, or
(b)₂-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O, and X₂ may be further optionally substituted with 1 or 2 substituents R₂₉ and R₃₀, wherein R₂₉ and R₃₀ are independently selected from alkyl, cycloalkyl, alkoxy, OH, oxo, CF₃, and halo; or

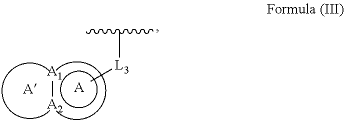

wherein:
L₃ is a single bond;
A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R₇ is selected from alkyl, alkoxy, and —(CH₂)₀₋₃-R₈,

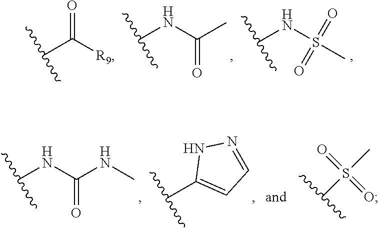

wherein R₈ is selected from cycloalkyl, heterocycloalkyl, wherein R₉ is selected from H, alkyl, OH, N(R₃₁)(R₃₂), and alkoxy;
wherein R₃₁ is H or small alkyl;
wherein R₃₂ is selected from H, small alkyl, and —(CH₂)₀₋₃heteroaryl;
wherein A′ may be optionally substituted with 1, 2, or 3 further substituents R₁₀, R₁₁, and R₁₇, wherein R₁₀, R₁₁, and R₁₇, are independently selected from alkyl, cycloalkyl, alkoxy, OH, oxo, CF₃, and halo, or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O.
B₄ can be N(R₂), Bs can be N, and B₆ can be C(R₃), wherein B₄ is connected to Bs via a single bond and Bs is connected to B₆ via a double bond, or B₄ can be C(R₂), Bs can be N, and B₆ can be N(R₃), wherein B₄ is connected to Bs via a double bond and Bs is connected to B₆ via a single bond; R₂ can be selected from —C(═O)—N(R₄)(R₅), —C(═O)—O—R₆,—CH₂—NH—S(═O)₂(cyclopropyl),—CH₂—O—CH₃,—CH₂—OH, and —CN; and
R₁ can be:
(i) Formula (IIb),

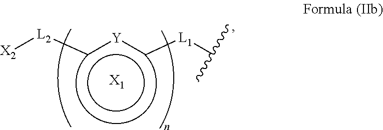

wherein:
n is 1;
Ly is a linker selected from a single bond, —(CH₂)₁₋₃—, —(CH)(alkyl)-, and —C(alkyl)(alkyl); Y is selected from C, N, O and S;
X₁ is phenyl or a 5- or 6-membered carbon-containing aromatic ring containing one, two or three ring members that are selected from N, S, and O;
wherein X₁ may be optionally substituted with 1 or 2 substituents independently selected from alkyl, cycloalkyl, alkoxy, OH, CF₃, and halo;
L₂ is a linker selected from a single bond, —(CH₂)₁₋₃—, —(CH)(alkyl)-, —C(alkyl)(alkyl)-, —C(═O)—, and —SO₂—; and
X₂ is either:
(a) —NR₂OR₂₁, wherein R₂₀ is selected from H, alkyl, and —(CH₂)₀₋₃-R_{22;}
wherein R₂₁ is selected from H, alkyl, and —(CH₂)₀₋₃-R₂₃;

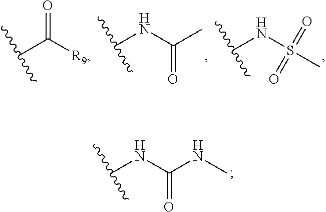

wherein R₂₂ is selected from cycloalkyl, heterocycloalkyl, aryl, heteroaryl,

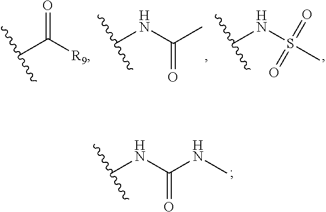

wherein R₂₃ is selected from cycloalkyl, heterocycloalkyl, aryl, heteroaryl, or
(b)₂-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O; or
(ii) Formula (III) comprising an aromatic ring A fused to a non-aromatic ring A′,

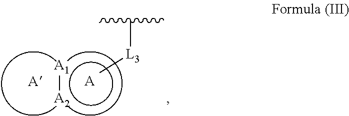

wherein:
L₃ is a single bond;
A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R₇ is selected from alkyl, alkoxy, and —(CH₂)₀₋₃-R₈,

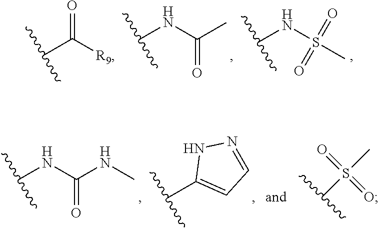

wherein R₈ is selected from cycloalkyl, heterocycloalkyl, wherein R₉ is selected from H, alkyl, OH, NH₂, and alkoxy;
wherein A′ may be optionally substituted with 1, 2, or 3 further substituents R₁₀, R₁₁, and R₁₇, wherein R₁₀, R₁₁, and R₁₇, are independently selected from alkyl, cycloalkyl, alkoxy, OH, oxo, CF₃, and halo, or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O.
B₄ can be C(R₂), Bs can be N, and B₆ can be N(R₃), wherein B₄ is connected to B₅ via a double bond and B₅ is connected to B₆ via a single bond; R₂ can be selected from —C(═O)—N(R₄)(R₅), —C(═O)—O—R₆,—CH₂—O—CH₃, and —CN; and
R₁ can be:
(i) Formula (IIa),

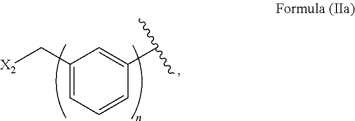

wherein:
n is 1;
X₂ is a non-aromatic, carbon-containing mono- or bi-cyclic ring containing 5, 6, or 7 ring members, wherein at least one ring member is independently selected from N, S, and O;
wherein X₂ may be optionally substituted with 1 or 2 substituents R₁₈ and R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O; or

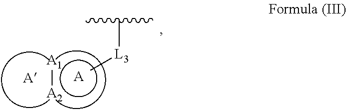

wherein:
L₃ is a single bond;
A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R₇ is selected from alkyl, alkoxy, and —(CH₂)₀₋₃-R₈,

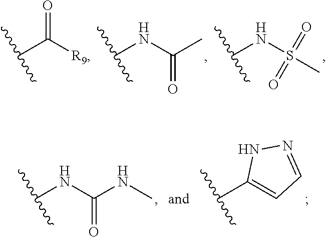

wherein R₈ is selected from cycloalkyl, heterocycloalkyl, wherein R₉ is selected from H, alkyl, OH, NH₂, and alkoxy;
wherein A′ may be optionally substituted with 1 or 2 further substituents R₁₀ and R₁₁, wherein R₁₀ and Ru are independently selected from alkyl, cycloalkyl, alkoxy, OH, oxo, CF₃, and halo, or wherein A′ may be optionally substituted with 1 further substituent R₂₄ selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O.
B₁ can be a carbon centre and B₂ can be a carbon centre, wherein B₃ is a single bond between B₂ and the nitrogen atom to which R₁ is attached and B₁ is connected to B₂ via a single bond, or B₁ can be a nitrogen centre and B₂ can be a carbon centre wherein B₁ is connected to B₂ via a double bond; B₄ can be N(R₂), B₅ can be N, and B₆ can be C(R₃), wherein B₄ is connected to B₅ via a single bond and B₅ is connected to B₆ via a double bond, or B₄ can be C(R₂), B₅ can be N, and B₆ can be N(R₃), wherein B₄ is connected to B₅ via a double bond and B₅ is connected to B₆ via a single bond; R₂ can be selected from —C(═O)—N(R₄)(R₅),

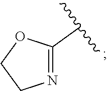 [SMILES: *C1=NCCO1]

—CH₂—NH—S(═O)₂(cyclopropyl),—CH₂—NH—C(═O) CH₃, —C(═O)—O—R₆,—CH₂—O—R₁₆,—CH₂—OH, —CN, and and
R₁ can be:
(i) Formula (IIb),

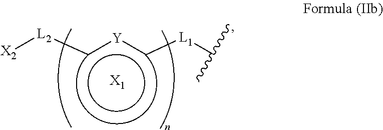

wherein:
n is 1;
L₁ is a linker selected from a single bond, —(CH₂)₁₋₃—, —(CH)(alkyl)-, and —C(alkyl)(alkyl)-;
Y is selected from C, N, O and S;
X₁ is phenyl or a 5- or 6-membered carbon-containing aromatic ring containing one, two or three ring members that are selected from N, S, and O;
wherein X₁ may be optionally substituted with 1 or 2 substituents independently selected from alkyl, cycloalkyl, alkoxy, OH, CF₃, and halo;
L₂ is a linker selected from a single bond, —(CH₂)₁₋₃—, —(CH)(alkyl)-, —C(alkyl)(alkyl)-, —C(═O)—, and —SO₂—; and X₂ is either:
(a) —NR₂OR₂₁, wherein R₂₀ is selected from H, alkyl, and —(CH₂)₀₋₃-R_{22;}
wherein R₂₁ is selected from H, alkyl, and —(CH₂)₀₋₃-R₂₃;

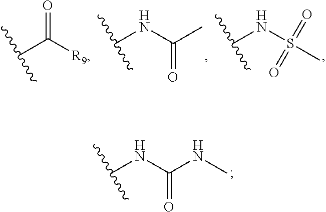

wherein R₂₂ is selected from cycloalkyl, heterocycloalkyl, aryl, heteroaryl,

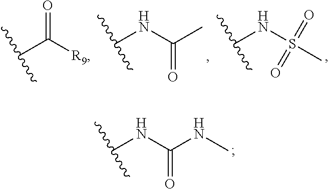

wherein R₂₃ is selected from cycloalkyl, heterocycloalkyl, aryl, heteroaryl, or
(b)₂-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected from alkyl, cycloalkyl, alkoxy, OH, Oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O, and X₂ may be further optionally substituted with 1 or 2 substituents R₂₉ and R₃₀, wherein R₂₉ and R₃₀ are independently selected from alkyl, cycloalkyl, alkoxy, OH, oxo, CF₃, and halo; or
(ii) Formula (III) comprising an aromatic ring A fused to a non-aromatic ring A′,

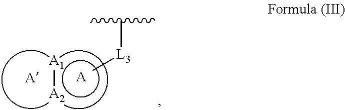

wherein:
L₃ is a single bond;
A₁ and A₂ are both carbon;
A is phenyl or a 5- or 6-membered carbon-containing aromatic ring containing one, two or three
wherein A may be optionally substituted with 1 or 2 substituents independently selected from alkyl, cycloalkyl, alkoxy, OH, CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R₇ is selected from alkyl, alkoxy, and —(CH₂)₀₋₃-R₈,

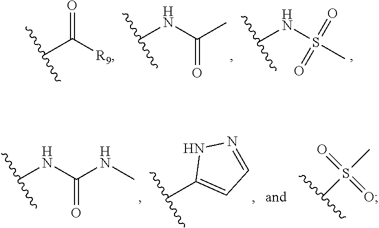

wherein R₈ is selected from cycloalkyl, heterocycloalkyl, wherein R₉ is selected from H, alkyl, OH, N(R₃₁)(R₃₂), and alkoxy;
wherein R₃₁ is H or small alkyl;
wherein R₃₂ is selected from H, small alkyl, and —(CH₂)₀₋₃heteroaryl;
wherein A′ may be optionally substituted with 1, 2, or 3 further substituents R₁₀, R₁₁, and R₁₇, wherein R₁₀, R₁₁, and R₁₇, are independently selected from alkyl, cycloalkyl, alkoxy, OH, oxo, CF₃, and halo, or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O.
B₁ can be a carbon centre and B₂ can be a carbon centre, wherein B₃ is a single bond between B₂ and the nitrogen atom to which R₁ is attached and B₁ is connected to B₂ via a single bond, or B₁ can be a nitrogen centre and B₂ can be a carbon centre wherein B₁ is connected to B₂ via a double bond; B₄ can be N(R₂), B₅ can be N, and B₆ can be C(R₃), wherein B₄ is connected to B₅ via a single bond and B₅ is connected to B₆ via a double bond, or B₄ can be C(R₂), B₅ can be N, and B₆ can be N(R₃), wherein B₄ is connected to B₅ via a double bond and B₅ is connected to B₆ via a single bond; R₂ can be selected from —C(═O)—N(R₄)(R₅), —CH₂—NH—S(═O)₂(cyclopropyl), —C(═O)—O—R₆,—CH₂—O—CH₃,—CH₂—OH, and —CN; and

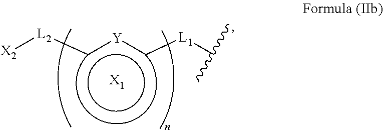

wherein:
n is 1;
L₁ is a linker selected from a single bond, —(CH₂)₁₋₃—, —(CH)(alkyl)-, and —C(alkyl)(alkyl)-; Y is selected from C, N, O and S; X₁ is phenyl or a 5- or 6-membered carbon-containing aromatic ring containing one, two or three ring members that are selected from N, S, and O;
wherein X₁ may be optionally substituted with 1 or 2 substituents independently selected from alkyl, cycloalkyl, alkoxy, OH, CF₃, and halo; L₂ is a linker selected from a single bond, —(CH₂)₁₋₃—, —(CH)(alkyl)-, —C(alkyl)(alkyl)-, —C(═O)—, and —SO₂—; and
X₂ is either:
(a) —NR₂OR₂₁, wherein R₂₀ is selected from H, alkyl, and —(CH₂)₀₋₃-R_{22;}
wherein R₂₁ is selected from H, alkyl, and —(CH₂)₀₋₃-R₂₃;

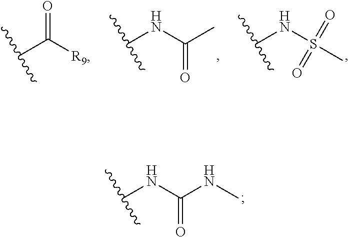

wherein R₂₂ is selected from cycloalkyl, heterocycloalkyl, aryl, heteroaryl,

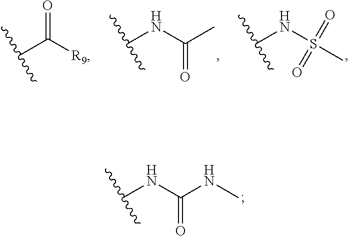

wherein R₂₃ is selected from cycloalkyl, heterocycloalkyl, aryl, heteroaryl, or
(b)₂-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O; or
(ii) Formula (III) comprising an aromatic ring A fused to a non-aromatic ring A′,

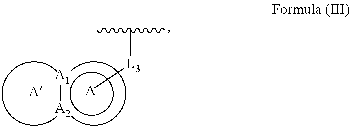

wherein:
L₃ is a single bond;
A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R₇ is selected from alkyl, alkoxy, and —(CH₂)₀₋₃-R₈,

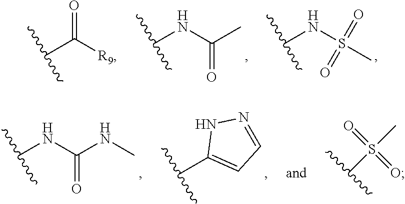

wherein R₈ is selected from cycloalkyl, heterocycloalkyl, wherein R₉ is selected from H, alkyl, OH, NH₂, and alkoxy;
wherein A′ may be optionally substituted with 1, 2, or 3 further substituents R₁₀, R₁₁, and R₁₇, wherein R₁₀, R₁₁, and R₁₇, are independently selected from alkyl, cycloalkyl, alkoxy, OH, oxo, CF₃, and halo, or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O.
B₁ can be a carbon centre and B₂ can be a carbon centre, wherein B₃ is a single bond between B₂ and the nitrogen atom to which R₁ is attached and B₁ is connected to B₂ via a single bond; B₄ can be C(R₂), B₅ can be N, and B₆ can be N(R₃), wherein B₄ is connected to Bs via a double bond and B₅ is connected to B₆ via a single bond; R₂ can be selected from —C(═O)—N(R₄)(R₅), —C(═O)—O—R₆,—CH₂—O—CH₃, and —CN; and R₁ can be:
(i) Formula (IIa),

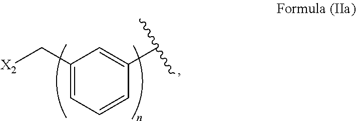

wherein:
n is 1;
X₂ is a non-aromatic, carbon-containing mono- or bi-cyclic ring containing 5, 6, or 7 ring members, wherein at least one ring member is independently selected from N, S, and O;
wherein X₂ may be optionally substituted with 1 or 2 substituents R₁₈ and R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O; or
(ii) Formula (III) comprising an aromatic ring A fused to a non-aromatic ring A′,

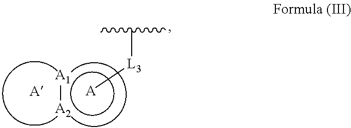

wherein:
L₃ is a single bond;
A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R₇ is selected from alkyl, alkoxy, and —(CH₂)₀₋₃-R_{8.}
wherein R₈ is selected from cycloalkyl, heterocycloalkyl,

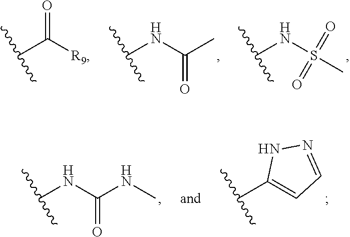

wherein R₈ is selected from H, alkyl, OH, NH₂, and alkoxy;
wherein A′ may be optionally substituted with 1 or 2 further substituents R₁₀ and R₁₁, wherein R₁₀ and R₁₁ are independently selected from alkyl, cycloalkyl, alkoxy, OH, oxo, CF₃, and halo, or wherein A′ may be optionally substituted with 1 further substituent R₂₄ selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O.
R₂ can be selected from —C(═O)—N(R₄)(R₅),—CH₂—NH—S(═O)₂(cyclopropyl),—CH₂—NH—C(═O) CH₃, —C(═O)—O—R₆, —

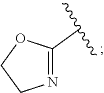 [SMILES: *C1=NCCO1]

CH₂—O—R₁₆,—CH₂—OH, —CN, and and
R₁ can be:
(i) Formula (IIb),

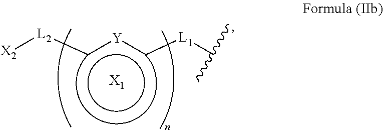

wherein:
n is 1; L₁ is a linker selected from a single bond, —(CH₂)₁₋₃—, —(CH)(alkyl)-, and —C(alkyl)(alkyl)-; Y is selected from C, N, O and S;
X₁ is phenyl or a 5- or 6-membered carbon-containing aromatic ring containing one, two or three ring members that are selected from N, S, and O;
wherein X₁ may be optionally substituted with 1 or 2 substituents independently selected from alkyl, cycloalkyl, alkoxy, OH, CF₃, and halo;
L₂ is a linker selected from a single bond, —(CH₂)₁₋₃—, —(CH)(alkyl)-, —C(alkyl)(alkyl)-, —C(═O)—, and —SO₂—; and
X₂ is either:
(a) NR₂OR₂₁, wherein R₂₀ is selected from H, alkyl, and —(CH₂)₀₋₃-R_{22;}
wherein R₂₁ is selected from H, alkyl, and —(CH₂)₀₋₃-R₂₃;

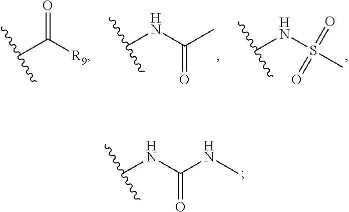

wherein R₂₂ is selected from cycloalkyl, heterocycloalkyl, aryl, heteroaryl,

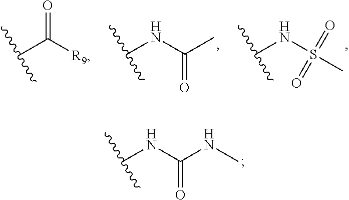

wherein R₂₃ is selected from cycloalkyl, heterocycloalkyl, aryl, heteroaryl, or
(b)₂-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O, and X₂ may be further optionally substituted with 1 or 2 substituents R₂₉ and R₃₀, wherein R₂₉ and R₃₀ are independently selected from alkyl, cycloalkyl, alkoxy, OH, oxo, CF₃, and halo; or

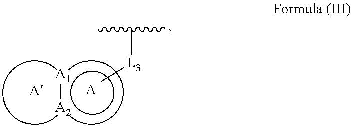

wherein:
L₃ is a single bond;
A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R₇ is selected from alkyl, alkoxy, and —(CH₂)₀₋₃-R₈,

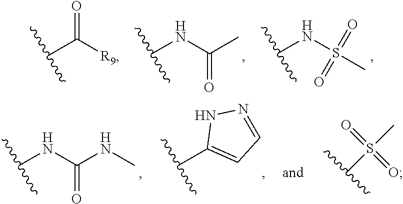

wherein R₈ is selected from cycloalkyl, heterocycloalkyl, wherein R₉ is selected from H, alkyl, OH, N(R₃₁)(R₃₂), and alkoxy;
wherein R₃₁ is H or small alkyl;
wherein R₃₂ is selected from H, small alkyl, and —(CH₂)₀₋₃heteroaryl;
wherein A′ may be optionally substituted with 1, 2, or 3 further substituents R₁₀, R₁₁, and R₁₇, wherein R₁₀, R₁₁, and R₁₇, are independently selected from alkyl, cycloalkyl, alkoxy, OH, oxo, CF₃, and halo, or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O.
R₂ can be selected from —C(═O)—N(R₄)(R₅),—CH₂—NH—S(═O)₂(cyclopropyl), —C(═O)—O—R₆,—CH₂—O—CH₃,—CH₂—OH, and —CN; and R₁ can be:
(i) Formula (IIb),

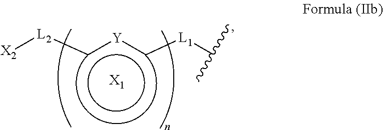

wherein:
n is 1;
L₁ is a linker selected from a single bond, —(CH₂)₁₋₃—, —(CH)(alkyl)-, and —C(alkyl)(alkyl); Y is selected from C, N, O and S;
X₁ is phenyl or a 5- or 6-membered carbon-containing aromatic ring containing one, two or three ring members that are selected from N, S, and O;
wherein X₁ may be optionally substituted with 1 or 2 substituents independently selected from alkyl, cycloalkyl, alkoxy, OH, CF₃, and halo;
L₂ is a linker selected from a single bond, —(CH₂)₁₋₃—, —(CH)(alkyl)-, —C(alkyl)(alkyl)-, —C(═O)—, and —SO₂—; and
X₂ is either:
(a) —NR₂OR₂₁, wherein R₂₀ is selected from H, alkyl, and —(CH₂)₀₋₃-R_{22;}
wherein R₂₁ is selected from H, alkyl, and —(CH₂)₀₋₃-R₂₃;

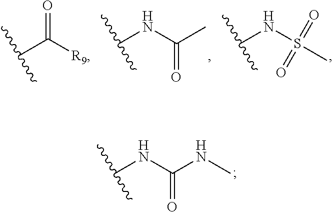

wherein R₂₂ is selected from cycloalkyl, heterocycloalkyl, aryl, heteroaryl,

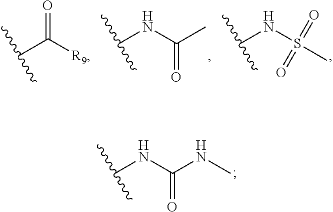

wherein R₂₃ is selected from cycloalkyl, heterocycloalkyl, aryl, heteroaryl, or
(b)₂-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O; or
(ii) Formula (III) comprising an aromatic ring A fused to a non-aromatic ring A′,

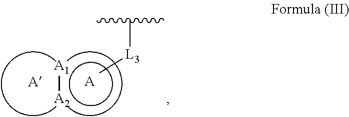

wherein:
L₃ is a single bond;
A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R₇ is selected from alkyl, alkoxy, and —(CH₂)₀₋₃-R₈,

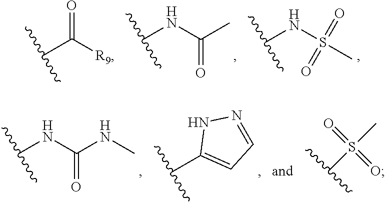

wherein R₈ is selected from cycloalkyl, heterocycloalkyl, wherein R₉ is selected from H, alkyl, OH, NH₂, and alkoxy;
wherein A′ may be optionally substituted with 1, 2, or 3 further substituents Rio, R₁₁, and R₁₇, wherein R₁₀, R₁₁, and R₁₇, are independently selected from alkyl, cycloalkyl, alkoxy, OH, oxo, CF₃, and halo, or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O.
R₂ can be selected from —C(═O)—N(R₄)(R₅), —C(═O)—O—R₆,—CH₂—O—CH₃, and —CN; and
R₁ can be:
(i) Formula (IIa),

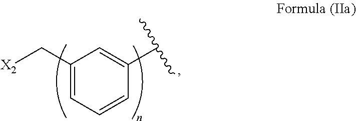

wherein:
n is 1;
X₂ is a non-aromatic, carbon-containing mono- or bi-cyclic ring containing 5, 6, or 7 ring members, wherein at least one ring member is independently selected from N, S, and O;
wherein X₂ may be optionally substituted with 1 or 2 substituents R₁₈ and R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O; or

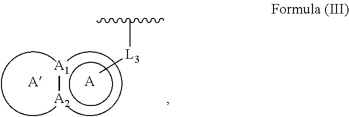

wherein:
L₃ is a single bond;
A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R₇ is selected from alkyl, alkoxy, and —(CH₂)₀₋₃-R₈,

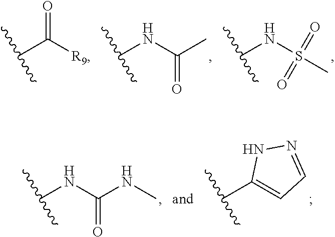

wherein R₈ is selected from cycloalkyl, heterocycloalkyl, wherein R₉ is selected from H, alkyl, OH, NH₂, and alkoxy;
wherein A′ may be optionally substituted with 1 or 2 further substituents R₁₀ and R₁₁, wherein R₁₀ and R₁₁ are independently selected from alkyl, cycloalkyl, alkoxy, OH, oxo, CF₃, and halo, or wherein A′ may be optionally substituted with 1 further substituent R₂₄ selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O.
B₁ can be a carbon centre and B₂ can be a carbon centre, wherein B₃ is a single bond between B₂ and the nitrogen atom to which R₁ is attached and B₁ is connected to B₂ via a single bond, or B₁ can be a nitrogen centre and B₂ can be a carbon centre wherein B₁ is connected to B₂ via a double bond;
R₂ can be selected from —C(═O)—N(R₄)(R₅),—CH₂—NH—S(═O)₂(cyclopropyl),—CH₂—NH—C(═O) CH₃, —C(═O)—O—R₆,

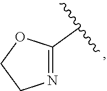 [SMILES: *C1=NCCO1]

—CH₂—O—R₁₆,—CH₂—OH, —CN, and R₄ is H or small alkyl, and wherein:
R₅ is selected from alkyl, OH, —S(═O)₂-(small alkyl), —(CH₂)₁₋₃—(C═O)—OH, —(CH₂)₀₋₃-cycloalkyl^{a}, —(CH₂)₀₋₃-heterocycloalkyl, —(CH(alkyl)-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, —(C(H)(heteroaryl))-alkyl, —(C(H)(alkyl))—(C═O)—OH, and —(C(H)(heteroaryl))—(C═O)—OR_{28;}
or R₄ and R₅ together with the nitrogen atom to which they are attached can form a carbon-containing 5-, or 6-membered saturated heterocyclic ring, optionally containing an additional heteroatom selected from N, and O, which may be optionally mono- or di-substituted with substituents selected from oxo, alkyl, alkoxy, OH, halo and CF₃; and
R₆ is H, small alkyl, and —(CH₂)-phenyl; and
R₁ can be:
(i) Formula (IIb),

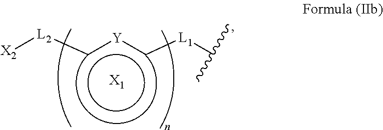

wherein:
n is 1;
L₁ is a linker selected from a single bond, —(CH₂)₁₋₃—, —(CH)(alkyl)-, and —C(alkyl)(alkyl)-; Y is selected from C, N, O and S;
X₁ is phenyl or a 5- or 6-membered carbon-containing aromatic ring containing one, two or three ring members that are selected from N, S, and O;
wherein X₁ may be optionally substituted with 1 or 2 substituents independently selected from alkyl, cycloalkyl, alkoxy, OH, CF₃, and halo;
L₂ is a linker selected from a single bond, —(CH₂)₁₋₃—, —(CH)(alkyl)-, —C(alkyl)(alkyl)-, —C(═O)—, and —SO₂—; and
X₂ is either:
(a) —NR₂OR₂₁, wherein R₂₀ is selected from H, alkyl, and —(CH₂)₀₋₃-R_{22;}
wherein R₂₁ is selected from H, alkyl, and —(CH₂)₀₋₃-R₂₃;

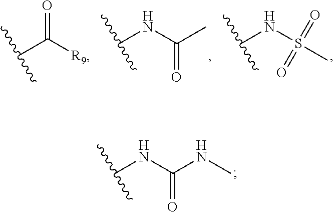

wherein R₂₂ is selected from cycloalkyl, heterocycloalkyl, aryl, heteroaryl,

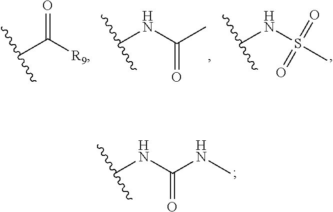

wherein R₂₃ is selected from cycloalkyl, heterocycloalkyl, aryl, heteroaryl, or
(b)₂-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O, and X₂ may be further optionally substituted with 1 or 2 substituents R₂₉ and R₃₀, wherein R₂₉ and R₃₀ are independently selected from alkyl, cycloalkyl, alkoxy, OH, oxo, CF₃, and halo; or
(ii) Formula (III) comprising an aromatic ring A fused to a non-aromatic ring A′,

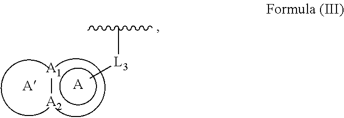

wherein:
L₃ is a single bond;
A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R₇ is selected from alkyl, alkoxy, and —(CH₂)₀₋₃-R₈,

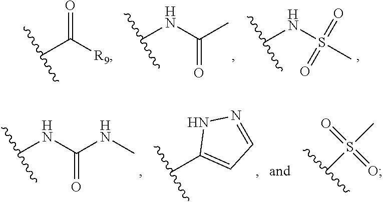

wherein R₈ is selected from cycloalkyl, heterocycloalkyl, wherein R₉ is selected from H, alkyl, OH, N(R₃₁)(R₃₂), and alkoxy;
wherein R₃₁ is H or small alkyl;
wherein R₃₂ is selected from H, small alkyl, and —(CH₂)₀₋₃heteroaryl;
wherein A′ may be optionally substituted with 1, 2, or 3 further substituents R₁₀, R₁₁, and R₁₇, wherein R₁₀, R₁₁, and R₁₇, are independently selected from alkyl, cycloalkyl, alkoxy, OH, oxo, CF₃, and halo, or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O.
B₁ can be a carbon centre and B₂ can be a carbon centre, wherein B₃ is a single bond between B₂ and the nitrogen atom to which R₁ is attached and B₁ is connected to B₂ via a single bond, or B₁ can be a nitrogen centre and B₂ can be a carbon centre wherein B₁ is connected to B₂ via a double bond;
R₂ can be selected from —C(═O)—N(R₄)(R₅),—CH₂—NH—S(═O)₂(cyclopropyl), —C(═O)—O—R₆,—CH₂—O—CH₃,—CH₂—OH, and —CN, wherein:
R₄ is H or small alkyl, and
R₅ is selected from alkyl, —(CH₂)₀₋₃-cycloalkyl, —(CH₂)₀₋₃-heterocycloalkyl, —(CH(alkyl))-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, and —(C(H)(heteroaryl))-alkyl;
or R₄ and R₅ together with the nitrogen atom to which they are attached can form a carbon-containing 5-, or 6-membered saturated heterocyclic ring, optionally containing an additional heteroatom selected from N, and O, which may be optionally mono- or di-substituted with substituents selected from oxo, alkyl, alkoxy, OH, halo and CF₃; and
R₆ is H, small alkyl, and —(CH₂)-phenyl; and

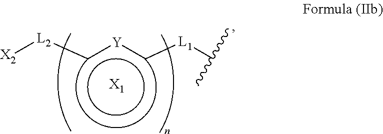

wherein:
n is 1;
L₁ is a linker selected from a single bond, —(CH₂)₁₋₃—, —(CH)(alkyl)-, and —C(alkyl)(alkyl); Y is selected from C, N, O and S;
X₁ is phenyl or a 5- or 6-membered carbon-containing aromatic ring containing one, two or three ring members that are selected from N, S, and O;
wherein X₁ may be optionally substituted with 1 or 2 substituents independently selected from alkyl, cycloalkyl, alkoxy, OH, CF₃, and halo;
L₂ is a linker selected from a single bond, —(CH₂)₁₋₃—, —(CH)(alkyl)-, —C(alkyl)(alkyl)-, —C(═O)—, and —SO₂—; and either:
wherein R₂₀ is selected from H, alkyl, and —(CH₂)₀₋₃-R_{22;}
wherein R₂₁ is selected from H, alkyl, and —(CH₂)₀₋₃-R_{23;}
X₂ is (a) —NR₂OR₂₁,

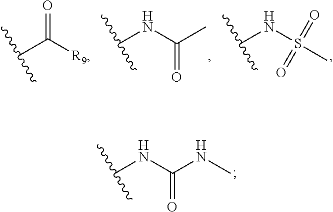

wherein R₂₂ is selected from cycloalkyl, heterocycloalkyl, aryl, heteroaryl,

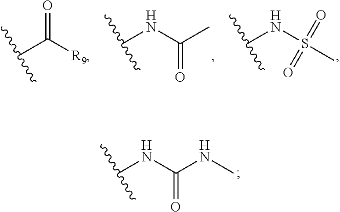

wherein R₂₃ is selected from cycloalkyl, heterocycloalkyl, aryl, heteroaryl, or
(b)₂-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O; or
(ii) Formula (III) comprising an aromatic ring A fused to a non-aromatic ring A′,

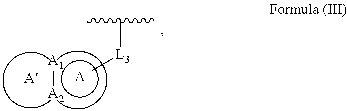

wherein:
L₃ is a single bond;
A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R₇ is selected from alkyl, alkoxy, and —(CH₂)₀₋₃-R₈,

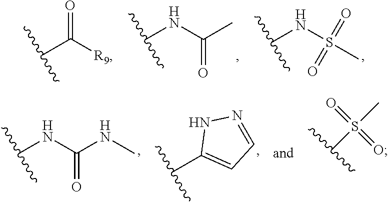

wherein R₈ is selected from cycloalkyl, heterocycloalkyl, wherein R₉ is selected from H, alkyl, OH, NH₂, and alkoxy;
wherein A′ may be optionally substituted with 1, 2, or 3 further substituents R₁₀, R₁₁, and R₁₇, wherein R₁₀, R₁₁, and R₁₇, are independently selected from alkyl, cycloalkyl, alkoxy, OH, oxo, CF₃, and halo, or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O.
B₁ can be a carbon centre and B₂ can be a carbon centre, wherein B₃ is a single bond between B₂ and the nitrogen atom to which R₁ is attached and B₁ is connected to B₂ via a single bond;
R₂ can be selected from —C(═O)—N(R₄)(R₅), —C(═O)—O—R₆,—CH₂—O—CH₃, and —CN, wherein:
R₄ is H or small alkyl, and
R₅ is selected from alkyl, —(CH₂)₀₋₃-cycloalkyl, —(CH₂)₀₋₃-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, and —(C(H)(heteroaryl))-alkyl;
or R₄ and R₅ together with the nitrogen atom to which they are attached can form a carbon-containing 5-, or 6-membered saturated heterocyclic ring, optionally containing an additional heteroatom selected from N, and O, which may be optionally mono- or di-substituted with substituents selected from oxo, alkyl, alkoxy, OH, halo and CF₃; and R₆ is H, small alkyl, and —(CH₂)-phenyl; and
R₁ can be:
(i) Formula (IIa),

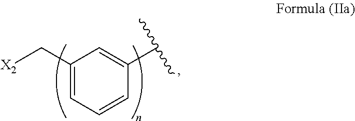

wherein:
n is 1;
X₂ is a non-aromatic, carbon-containing mono- or bi-cyclic ring containing 5, 6, or 7 ring members, wherein at least one ring member is independently selected from N, S, and O;
wherein X₂ may be optionally substituted with 1 or 2 substituents R₁₈ and R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O; or

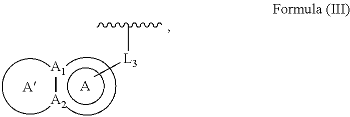

wherein:
L₃ is a single bond;
A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R₇ is selected from alkyl, alkoxy, and —(CH₂)₀₋₃-R₈,

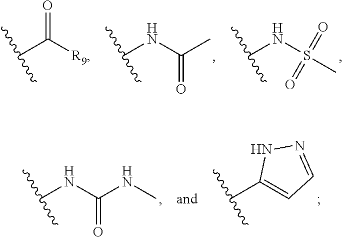

wherein R₈ is selected from cycloalkyl, heterocycloalkyl, wherein R₉ is selected from H, alkyl, OH, NH₂, and alkoxy;
wherein A′ may be optionally substituted with 1 or 2 further substituents R₁₀ and R₁₁, wherein R₁₀ and Ru are independently selected from alkyl, cycloalkyl, alkoxy, OH, oxo, CF₃, and halo, or wherein A′ may be optionally substituted with 1 further substituent R₂₄ selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O.
B₄ can be N(R₂), Bs can be N, and B₆ can be C(R₃), wherein B₄ is connected to B₅ via a single bond and B₅ is connected to B₆ via a double bond, or B₄ can be C(R₂), B₅ can be N, and B₆ can be N(R₃), wherein B₄ is connected to B₅ via a double bond and B₅ is connected to B₆ via a single bond;
R₂ can be selected from —C(═O)—N(R₄)(R₅), —C(═O)—O—R₆,—CH₂—NH—C(═O) CH₃,—CH₂—NH—S(═O)₂(cyclopropyl),

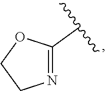 [SMILES: *C1=NCCO1]

—CH₂—O—R₁₆,—CH₂—OH, —CN, and wherein:
R₄ is H or small alkyl, and
R₅ is selected from alkyl, OH, —S(═O)₂-(small alkyl), —(CH₂)₁₋₃—(C═O)—OH, —(CH₂)₀₋₃-cycloalkyl^{a}, —(CH₂)₀₋₃-heterocycloalkyl, —(CH(alkyl)-heterocycloalkyl, —(CH₂)₀₋₃-polycycloalkyl, —(CH₂)₀₋₃-aryl, —(C(H)(phenyl)-alkyl, —(CH₂)₀₋₃heteroaryl, —(C(H)(heteroaryl)-alkyl, —(C(H)(alkyl)—(C═O)—OH, and —(C(H)(heteroaryl))—(C═O)—OR_{28;}
or R₄ and R₅ together with the nitrogen atom to which they are attached can form a carbon-containing 4-, 5-, or 6-membered saturated heterocyclic ring, optionally containing an additional heteroatom selected from N, and O, which may be optionally mono- or di-substituted with substituents selected from oxo, alkyl, alkoxy, OH, halo and CF₃; and
R₆ is H, small alkyl, and —(CH₂)-phenyl; and
R₁ can be:
(i) Formula (IIb),

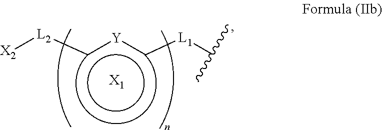

wherein:
n is 1;
L₁ is a linker selected from a single bond, —(CH₂)₁₋₃—, —(CH)(alkyl)-, and —C(alkyl)(alkyl)-; Y is selected from C, N, O and S;
X₁ is phenyl or a 5- or 6-membered carbon-containing aromatic ring containing one, two or three ring members that are selected from N, S, and O;
wherein X₁ may be optionally substituted with 1 or 2 substituents independently selected from alkyl, cycloalkyl, alkoxy, OH, CF₃, and halo;
L₂ is a linker selected from a single bond, —(CH₂)₁₋₃—, —(CH)(alkyl)-, —C(alkyl)(alkyl)-, —C(═O)—, and —SO₂—; and
X₂ is either:
(a) —NR₂OR₂₁, wherein R₂₀ is selected from H, alkyl, and —(CH₂)₀₋₃-R_{22;}
wherein R₂₁ is selected from H, alkyl, and —(CH₂)₀₋₃-R₂₃;

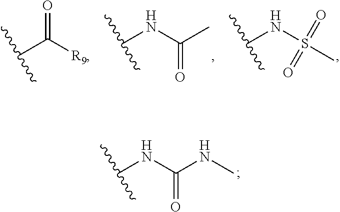

wherein R₂₂ is selected from cycloalkyl, heterocycloalkyl, aryl, heteroaryl,

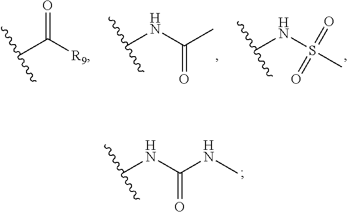

wherein R₂₃ is selected from cycloalkyl, heterocycloalkyl, aryl, heteroaryl, or
(b)₂-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected from alkyl, cycloalkyl, alkoxy, OH, Oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O, and X₂ may be further optionally substituted with 1 or 2 substituents R₂₉ and R₃₀, wherein R₂₉ and R₃₀ are independently selected from alkyl, cycloalkyl, alkoxy, OH, oxo, CF₃, and halo; or
(ii) Formula (III) comprising an aromatic ring A fused to a non-aromatic ring A′,

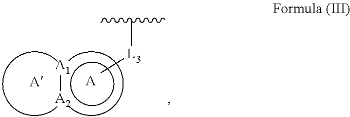

wherein:
L₃ is a single bond;
A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R₇ is selected from alkyl, alkoxy, and —(CH₂)₀₋₃-R₈,

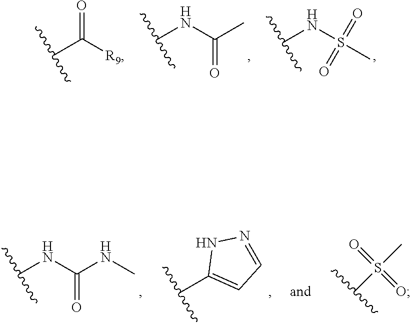

wherein R₈ is selected from cycloalkyl, heterocycloalkyl, wherein R₈ is selected from H, alkyl, OH, N(R₃₁)(R₃₂), and alkoxy;
wherein R₃₁ is H or small alkyl;
wherein R₃₂ is selected from H, small alkyl, and —(CH₂)₀₋₃heteroaryl;
wherein A′ may be optionally substituted with 1, 2, or 3 further substituents R₁₀, R₁₁, and R₁₇, wherein R₁₀, R₁₁, and R₁₇, are independently selected from alkyl, cycloalkyl, alkoxy, OH, oxo, CF₃, and halo, or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O.
B₄ can be N(R₂), B₅ can be N, and B₆ can be C(R₃), wherein B₄ is connected to B₅ via a single bond and B₅ is connected to B₆ via a double bond, or B₄ can be C(R₂), B₅ can be N, and B₆ can be N(R₃), wherein B₄ is connected to B₅ via a double bond and B₅ is connected to B₆ via a single bond; R₂ can be selected from —C(═O)—N(R₄)(R₅), —C(═O)—O—R₆,—CH₂—NH—S(═O)₂(cyclopropyl),—CH₂—O—CH₃,—CH₂—OH, and —CN, wherein:
R₄ is H or small alkyl, and
R₅ is selected from alkyl, —(CH₂)₀₋₃-cycloalkyl, —(CH₂)₀₋₃-heterocycloalkyl, —(CH(alkyl))-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, and —(C(H)(heteroaryl))-alkyl;
or R₄ and R₅ together with the nitrogen atom to which they are attached can form a carbon-containing 5-, or 6-membered saturated heterocyclic ring, optionally containing an additional heteroatom selected from N, and O, which may be optionally mono- or di-substituted with substituents selected from oxo, alkyl, alkoxy, OH, halo and CF₃; and
R₆ is H, small alkyl, and —(CH₂)-phenyl; and
R₁ can be:
(i) Formula (IIb),

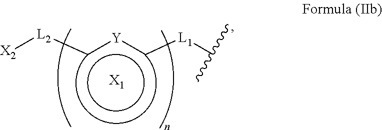

wherein:
n is 1;
L₁ is a linker selected from a single bond, —(CH₂)₁₋₃—, —(CH)(alkyl)-, and —C(alkyl)(alkyl)-; Y is selected from C, N, O and S;
X₁ is phenyl or a 5- or 6-membered carbon-containing aromatic ring containing one, two or three ring members that are selected from N, S, and O;
wherein X₁ may be optionally substituted with 1 or 2 substituents independently selected from alkyl, cycloalkyl, alkoxy, OH, CF₃, and halo;
L₂ is a linker selected from a single bond, —(CH₂)₁₋₃—, —(CH)(alkyl)-, —C(alkyl)(alkyl)-, —C(═O)—, and —SO₂—; and
X₂ is either:
(a) —NR₂OR₂₁, wherein R₂₀ is selected from H, alkyl, and —(CH₂)₀₋₃-R_{22;}
wherein R₂₁ is selected from H, alkyl, and —(CH₂)₀₋₃-R₂₃;

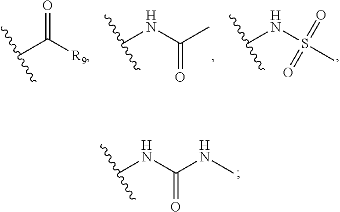

wherein R₂₂ is selected from cycloalkyl, heterocycloalkyl, aryl, heteroaryl,

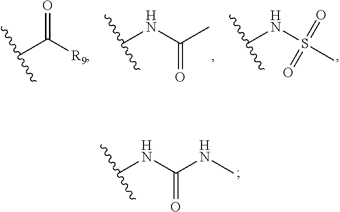

wherein R₂₃ is selected from cycloalkyl, heterocycloalkyl, aryl, heteroaryl, or
(b)₂-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected from alkyl, cycloalkyl, OH, alkoxy, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O; or
(ii) Formula (III) comprising an aromatic ring A fused to a non-aromatic ring A′,

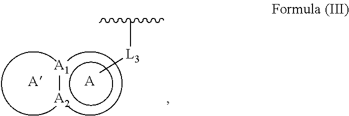

wherein:
L₃ is a single bond;
A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R₇ is selected from alkyl, alkoxy, and —(CH₂)₀₋₃-R₈,

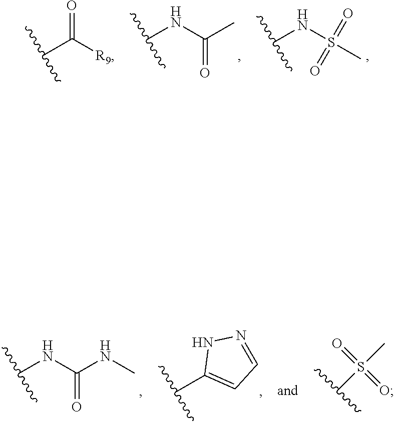

wherein R₈ is selected from cycloalkyl, heterocycloalkyl, wherein R₉ is selected from H, alkyl, OH, NH₂, and alkoxy;
wherein A′ may be optionally substituted with 1, 2, or 3 further substituents R₁₀, R₁₁, and R₁₇, wherein R₁₀, R₁₁, and R₁₇, are independently selected from alkyl, cycloalkyl, alkoxy, OH, oxo, CF₃, and halo, or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O.
B₄ can be C(R₂), B₅ can be N, and B₆ can be N(R₃), wherein B₄ is connected to B₅ via a double bond and B₅ is connected to B₆ via a single bond;
R₂ can be selected from —C(═O)—N(R₄)(R₅), —C(═O)—O—R₆,—CH₂—O—CH₃, and —CN, wherein:
R₄ is H or small alkyl, and
R₅ is selected from alkyl, —(CH₂)₀₋₃-cycloalkyl, —(CH₂)₀₋₃-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, and —(C(H)(heteroaryl))-alkyl;
or R₄ and R₅ together with the nitrogen atom to which they are attached can form a carbon-containing 5-, or 6-membered saturated heterocyclic ring, optionally containing an additional heteroatom selected from N, and O, which may be optionally mono- or di-substituted with substituents selected from oxo, alkyl, alkoxy, OH, halo and CF₃; and
R₆ is H, small alkyl, and —(CH₂)-phenyl; and
R₁ can be:
(i) Formula (IIa),

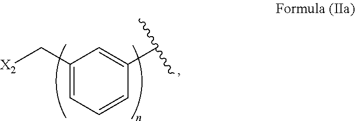

wherein:
n is 1;
X₂ is a non-aromatic, carbon-containing mono- or bi-cyclic ring containing 5, 6, or 7 ring members, wherein at least one ring member is independently selected from N, S, and O;
wherein X₂ may be optionally substituted with 1 or 2 substituents R₁₈ and R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O; or
(ii) Formula (III) comprising an aromatic ring A fused to a non-aromatic ring A′,

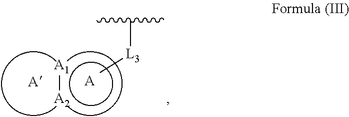

wherein:
L₃ is a single bond;
A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R₇ is selected from alkyl, alkoxy, and —(CH₂)₀₋₃-R₈,

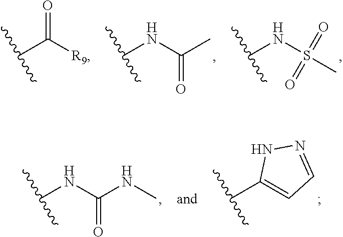

wherein R₈ is selected from cycloalkyl, heterocycloalkyl, wherein R₉ is selected from H, alkyl, OH, NH₂, and alkoxy;
wherein A′ may be optionally substituted with 1 or 2 further substituents R₁₀ and R₁₁, wherein R₁₀ and R₁₁ are independently selected from alkyl, cycloalkyl, alkoxy, OH, oxo, CF₃, and halo, or wherein A′ may be optionally substituted with 1 further substituent R₂₄ selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O.
B₁ can be a carbon centre and B₂ can be a carbon centre, wherein B₃ is a single bond between B₂ and the nitrogen atom to which R₁ is attached and B₁ is connected to B₂ via a single bond, or B₁ can be a nitrogen centre and B₂ can be a carbon centre wherein B₁ is connected to B₂ via a double bond; B₄ can be N(R₂), B₅ can be N, and B₆ can be C(R₃), wherein B₄ is connected to B₅ via a single bond and B₅ is connected to B₆ via a double bond, or B₄ can be C(R₂), B₅ can be N, and B₆ can be N(R₃), wherein B₄ is connected to B₅ via a double bond and B₅ is connected to B₆ via a single bond;
R₂ can be selected from —C(═O)—N(R₄)(R₅), —C(═O)—O—R₆,—CH₂—NH—C(═O) CH₃,—CH₂—NH—S(═O)₂(cyclopropyl),

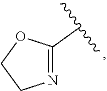 [SMILES: *C1=NCCO1]

—CH₂—O—R₁₆,—CH₂—OH, and —CN, and wherein:
R₄ is H or small alkyl, and
R₅ is selected from alkyl, OH, —S(═O)₂-(small alkyl), —(CH₂)₁₋₃—(C═O)—OH, —(CH₂)₀₋₃-cycloalkyl^{a}, —(CH₂)₀₋₃-heterocycloalkyl, —(CH(alkyl))-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, —(C(H)(heteroaryl))-alkyl, —(C(H)(alkyl))—(C═O)—OH, and —(C(H)(heteroaryl))—(C═O)—OR_{28;}
or R₄ and R₅ together with the nitrogen atom to which they are attached can form a carbon-containing 4-, 5-, or 6-membered saturated heterocyclic ring, optionally containing an additional heteroatom selected from N, and O, which may be optionally mono- or di-substituted with substituents selected from oxo, alkyl, alkoxy, OH, halo and CF₃; and R₆ is H, small alkyl, and —(CH₂)-phenyl; and
R₁ can be:

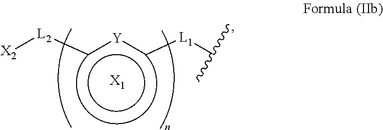

wherein:
n is 1;
L₁ is a linker selected from a single bond, —(CH₂)₁₋₃—, —(CH)(alkyl)-, and —C(alkyl)(alkyl)-; Y is selected from C, N, O and S;
X₁ is phenyl or a 5- or 6-membered carbon-containing aromatic ring containing one, two or three ring members that are selected from N, S, and O;
wherein X₁ may be optionally substituted with 1 or 2 substituents independently selected from alkyl, cycloalkyl, alkoxy, OH, CF₃, and halo;
L₂ is a linker selected from a single bond, —(CH₂)₁₋₃—, —(CH)(alkyl)-, —C(alkyl)(alkyl)-, —C(═O)—, and —SO₂—; and
X₂ is either: (a) —NR₂OR₂₁, wherein R₂₀ is selected from H, alkyl, and —(CH₂)₀₋₃-R_{22;}
wherein R₂₁ is selected from H, alkyl, and —(CH₂)₀₋₃-R₂₃;

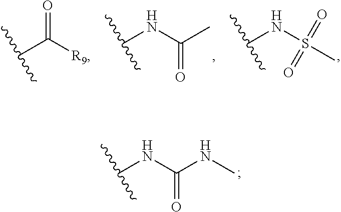

wherein R₂₂ is selected from cycloalkyl, heterocycloalkyl, aryl, heteroaryl,

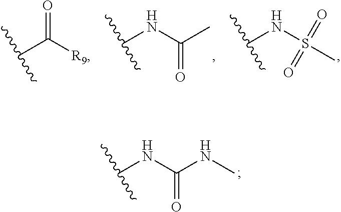

wherein R₂₃ is selected from cycloalkyl, heterocycloalkyl, aryl, heteroaryl, or
(b)₂-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected from alkyl, cycloalkyl, OH, alkoxy, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O, and X₂ may be further optionally substituted with 1 or 2 substituents R₂₉ and R₃₀, wherein R₂₉ and R₃₀ are independently selected from alkyl, cycloalkyl, alkoxy, OH, oxo, CF₃, and halo; or

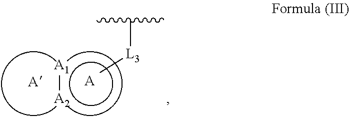

wherein:
L₃ is a single bond;
A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R, is selected from alkyl, alkoxy, and —(CH₂)₀₋₃-R₈,

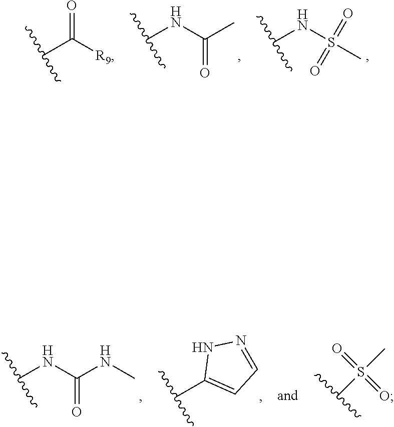

wherein R₈ is selected from cycloalkyl, heterocycloalkyl, wherein R₉ is selected from H, alkyl, OH, N(R₃₁)(R₃₂), and alkoxy;
wherein R₃₁ is H or small alkyl;
wherein R₃₂ is selected from H, small alkyl, and —(CH₂)₀₋₃heteroaryl;
wherein A′ may be optionally substituted with 1, 2, or 3 further substituents R₁₀, R₁₁, and R₁₇, wherein R₁₀, R₁₁, and R₁₇, are independently selected from alkyl, cycloalkyl, alkoxy, OH, oxo, CF₃, and halo, or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O.
B₁ can be a carbon centre and B₂ can be a carbon centre, wherein B₃ is a single bond between B₂ and the nitrogen atom to which R₁ is attached and B₁ is connected to B₂ via a single bond, or B₁ can be a nitrogen centre and B₂ can be a carbon centre wherein B₁ is connected to B₂ via a double bond; B₄ can be N(R₂), B₅ can be N, and B₆ can be C(R₃), wherein B₄ is connected to B₅ via a single bond and B₅ is connected to B₆ via a double bond, or B₄ can be C(R₂), B₅ can be N, and B₆ can be N(R₃), wherein B₄ is connected to B₅ via a double bond and B₅ is connected to B₆ via a single bond;
R₂ can be selected from —C(═O)—N(R₄)(R₅), —C(═O)—O—R₆,—CH₂—NH—S(═O)₂(cyclopropyl),—CH₂—O—CH₃,—CH₂—OH, and —CN, wherein:
R₄ is H or small alkyl, and
R₅ is selected from alkyl, —(CH₂)₀₋₃-cycloalkyl, —(CH₂)₀₋₃-heterocycloalkyl, —(CH(alkyl))-heterocycloalkyl, —(CH₂)₀₋₃-polycycloalkyl, —(CH₂)₀₋₃-aryl, —(C(H)(phenyl)-alkyl, —(CH₂)₀₋₃heteroaryl, and —(C(H)(heteroaryl))-alkyl;
or R₄ and R₅ together with the nitrogen atom to which they are attached can form a carbon-containing 5-, or 6-membered saturated heterocyclic ring, optionally containing an additional heteroatom selected from N, and O, which may be optionally mono- or di-substituted with substituents selected from oxo, alkyl, alkoxy, OH, halo and CF₃; and R₆ is H, small alkyl, and —(CH₂)-phenyl; and
R₁ can be:
(i) Formula (IIb),

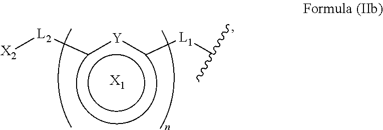

wherein:
n is 1;
L₁ is a linker selected from a single bond, —(CH₂)₁₋₃—, —(CH)(alkyl)-, and —C(alkyl)(alkyl)-; Y is selected from C, N, O and S;
X₁ is phenyl or a 5- or 6-membered carbon-containing aromatic ring containing one, two or three ring members that are selected from N, S, and O;
wherein X₁ may be optionally substituted with 1 or 2 substituents independently selected from alkyl, cycloalkyl, alkoxy, OH, CF₃, and halo;
L₂ is a linker selected from a single bond, —(CH₂)₁₋₃—, —(CH)(alkyl)-, —C(alkyl)(alkyl)-, —C(═O)—, and —SO₂—; and
X₂ is either:
(a) —NR₂OR₂₁, wherein R₂₀ is selected from H, alkyl, and —(CH₂)₀₋₃-R_{22;}
wherein R₂₁ is selected from H, alkyl, and —(CH₂)₀₋₃-R₂₃; wherein R₂₂ is selected from cycloalkyl, heterocycloalkyl, aryl, heteroaryl,

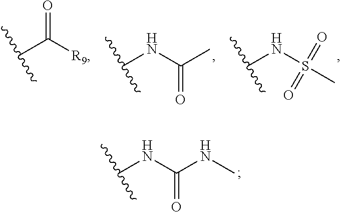

wherein R₂₃ is selected from cycloalkyl, heterocycloalkyl, aryl, heteroaryl,

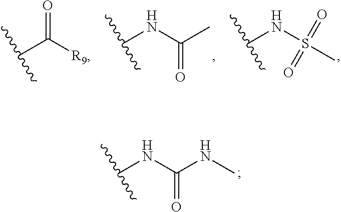

or
(b)₂-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O; or
(ii) Formula (III) comprising an aromatic ring A fused to a non-aromatic ring A′,

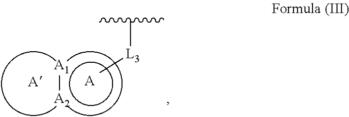

wherein:
L₃ is a single bond;
A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R₇ is selected from alkyl, alkoxy, and —(CH₂)₀₋₃-R₈,

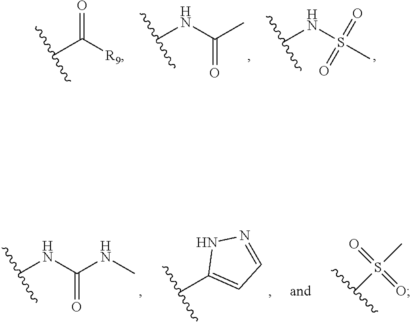

wherein R₈ is selected from cycloalkyl, heterocycloalkyl, wherein R₉ is selected from H, alkyl, OH, NH₂, and alkoxy;
wherein A′ may be optionally substituted with 1, 2, or 3 further substituents R₁₀, R₁₁, and R₁₇, wherein R₁₀, R₁₁, and R₁₇, are independently selected from alkyl, cycloalkyl, alkoxy, OH, oxo, CF₃, and halo, or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O.
B₁ can be a carbon centre and B₂ can be a carbon centre, wherein B₃ is a single bond between B₂ and the nitrogen atom to which R₁ is attached and B₁ is connected to B₂ via a single bond; B₄ can be C(R₂), B₅ can be N, and B₆ can be N(R₃), wherein B₄ is connected to B₅ via a double bond and B₅ is connected to B₆ via a single bond; R₂ can be selected from —C(═O)—N(R₄)(R₅), —C(═O)—O—R₆,—CH₂—O—CH₃, and —CN, wherein:
R₄ is H or small alkyl, and
R is selected from alkyl, —(CH₂)₀₋₃-cycloalkyl, —(CH₂)₀₋₃-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, and —(C(H)(heteroaryl))-alkyl;
or R₄ and R₅ together with the nitrogen atom to which they are attached can form a carbon-containing 5-, or 6-membered saturated heterocyclic ring, optionally containing an additional heteroatom selected from N, and O, which may be optionally mono- or di-substituted with substituents selected from oxo, alkyl, alkoxy, OH, halo and CF₃; and R₆ is H, small alkyl, and —(CH₂)-phenyl; and
R₁ can be:
(i) Formula (IIa),

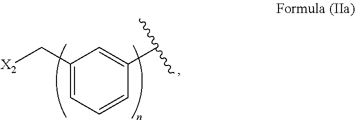

wherein:
n is 1;
X₂ is a non-aromatic, carbon-containing mono- or bi-cyclic ring containing 5, 6, or 7 ring members, wherein at least one ring member is independently selected from N, S, and O;
wherein X₂ may be optionally substituted with 1 or 2 substituents R₁₈ and R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O; or
(ii) Formula (III) comprising an aromatic ring A fused to a non-aromatic ring A′,

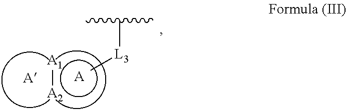

wherein:
L₃ is a single bond;
A₁ and Az are both carbon;
A is phenyl or a 5- or 6-membered carbon-containing aromatic ring containing one, two or three
wherein A may be optionally substituted with 1 or 2 substituents independently selected from alkyl, cycloalkyl, alkoxy, OH, CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R₇ is selected from alkyl, alkoxy, and —(CH₂)₀₋₃-R₈, wherein R₈ is selected from cycloalkyl, heterocycloalkyl,

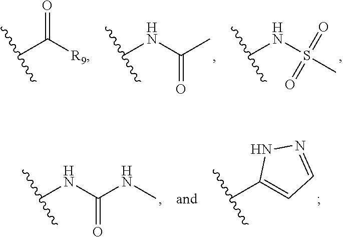

wherein R₉ is selected from H, alkyl, OH, NH₂, and alkoxy;
wherein A’ may be optionally substituted with 1 or 2 further substituents R₁₀ and R₁₁, wherein R₁₀ and R₁₁ are independently selected from alkyl, cycloalkyl, alkoxy, OH, oxo, CF₃, and halo, or wherein A′ may be optionally substituted with 1 further substituent R₂₄ selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O.
R₂ can be selected from —C(═O)—N(R₄)(R₅), —C(═O)—O—R₆,—CH₂—NH—C(═O) CH₃,—CH₂—NH—S(═O)₂(cyclopropyl),

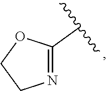 [SMILES: *C1=NCCO1]

—CH₂—O—R₁₆,—CH₂—OH, —CN, and wherein:
R₄ is H or small alkyl, and
R₅ is selected from alkyl, OH, —S(═O)₂-(small alkyl), —(CH₂)₁₋₃—(C═O)—OH, —(CH₂)₀₋₃-cycloalkyl^{a}, —(CH₂)₀₋₃-heterocycloalkyl, —(CH(alkyl)-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, —(C(H)(heteroaryl))-alkyl, —(C(H)(alkyl))—(C═O)—OH, and —(C(H)(heteroaryl))—(C═O)—OR_{28;}
or R₄ and R₅ together with the nitrogen atom to which they are attached can form a carbon-containing 4-, 5-, or 6-membered saturated heterocyclic ring, optionally containing an additional heteroatom selected from N, and O, which may be optionally mono- or di-substituted with substituents selected from oxo, alkyl, alkoxy, OH, halo and CF₃; and R₆ is H, small alkyl, and —(CH₂)-phenyl; and

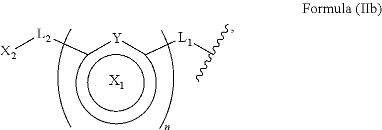

wherein:
n is 1;
L₁ is a linker selected from a single bond, —(CH₂)₁₋₃—, —(CH)(alkyl)-, and —C(alkyl)(alkyl)-; Y is selected from C, N, O and S; X₁ is phenyl or a 5- or 6-membered carbon-containing aromatic ring containing one, two or three ring members that are selected from N, S, and O;
wherein X₁ may be optionally substituted with 1 or 2 substituents independently selected from alkyl, cycloalkyl, alkoxy, OH, CF₃, and halo; L₂ is a linker selected from a single bond, —(CH₂)₁₋₃—, —(CH)(alkyl)-, —C(alkyl)(alkyl)-, —C(═O)—, and —SO₂—; and
X₂ is either:
(a) —NR₂OR₂₁, wherein R₂₀ is selected from H, alkyl, and —(CH₂)₀₋₃-R_{22;}
wherein R₂₁ is selected from H, alkyl, and —(CH₂)₀₋₃-R₂₃;

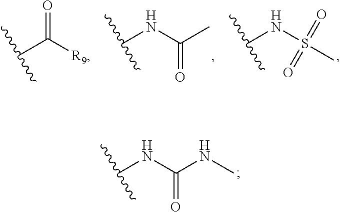

wherein R₂₂ is selected from cycloalkyl, heterocycloalkyl, aryl, heteroaryl,

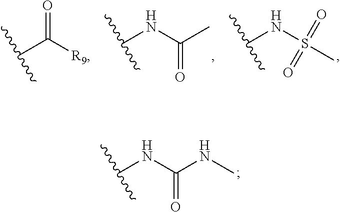

wherein R₂₃ is selected from cycloalkyl, heterocycloalkyl, aryl, heteroaryl, or
(b)₂-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected from alkyl, cycloalkyl, alkoxy, OH, Oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O, and X₂ may be further optionally substituted with 1 or 2 substituents R₂₉ and R₃₀, wherein R₂₉ and R₃₀ are independently selected from alkyl, cycloalkyl, alkoxy, OH, oxo, CF₃, and halo; or
(ii) Formula (III) comprising an aromatic ring A fused to a non-aromatic ring A′,

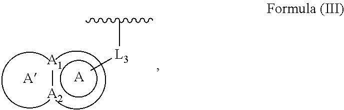

wherein:
L₃ is a single bond;
A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R₇ is selected from alkyl, alkoxy, and —(CH₂)₀₋₃-R₈,

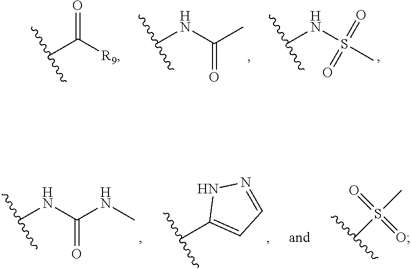

wherein R₈ is selected from cycloalkyl, heterocycloalkyl, wherein R₉ is selected from H, alkyl, OH, NH, and alkoxy;
wherein R₃₁ is H or small alkyl;
wherein R₃₂ is selected from H, small alkyl, and —(CH₂)₀₋₃heteroaryl;
wherein A′ may be optionally substituted with 1, 2, or 3 further substituents R₁₀, R₁₁, and R₁₇, wherein R₁₀, R₁₁, and R₁₇, are independently selected from alkyl, cycloalkyl, alkoxy, OH, oxo, CF₃, and halo, or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O.
R₂ can be selected from —C(═O)—N(R₄)(R₅), —C(═O)—O—R₆,—CH₂—NH—S(═O)₂(cyclopropyl),—CH₂—O—CH₃,—CH₂—OH, and —CN, wherein:
R₄ is H or small alkyl, and
R₅ is selected from alkyl, —(CH₂)₀₋₃-cycloalkyl, —(CH₂)₀₋₃-heterocycloalkyl, —(CH(alkyl)-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, and —(C(H)(heteroaryl))-alkyl;
or R₄ and R₅ together with the nitrogen atom to which they are attached can form a carbon-containing 5-, or 6-membered saturated heterocyclic ring, optionally containing an additional heteroatom selected from N, and O, which may be optionally mono- or di-substituted with substituents selected from oxo, alkyl, alkoxy, OH, halo and CF₃; and
R₆ is H, small alkyl, and —(CH₂)-phenyl; and
R₁ can be:
(i) Formula (IIb),

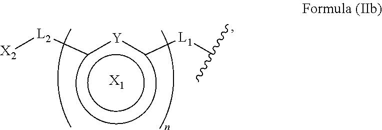

wherein:
n is 1;
L₁ is a linker selected from a single bond, —(CH₂)₁₋₃—, —(CH)(alkyl)-, and —C(alkyl)(alkyl)-; Y is selected from C, N, O and S;
X₁ is phenyl or a 5- or 6-membered carbon-containing aromatic ring containing one, two or three
wherein X₁ may be optionally substituted with 1 or 2 substituents independently selected from alkyl, cycloalkyl, alkoxy, OH, CF₃, and halo;
L₂ is a linker selected from a single bond, —(CH₂)₁₋₃—, —(CH)(alkyl)-, —C(alkyl)(alkyl)-, —C(═O)—, and —SO₂—; and
X₂ is either:
wherein R₂₀ is selected from H, alkyl, and —(CH₂)₀₋₃-R_{22;}
wherein R₂₁ is selected from H, alkyl, and —(CH₂)₀₋₃-R_{23;}
(a) —NR₂OR₂₁,

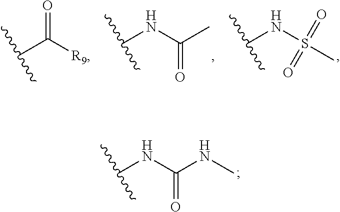

wherein R₂₂ is selected from cycloalkyl, heterocycloalkyl, aryl, heteroaryl,

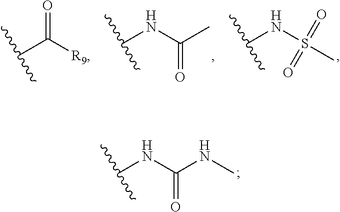

wherein R₂₃ is selected from cycloalkyl, heterocycloalkyl, aryl, heteroaryl, or
(b)₂-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected from alkyl, cycloalkyl, OH, oxo, alkoxy, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O; or
(ii) Formula (III) comprising an aromatic ring A fused to a non-aromatic ring A′,

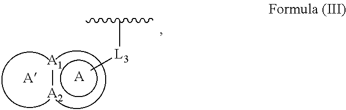

wherein:
L₃ is a single bond;
A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R₇ is selected from alkyl, alkoxy, and —(CH₂)₀₋₃-R₈,

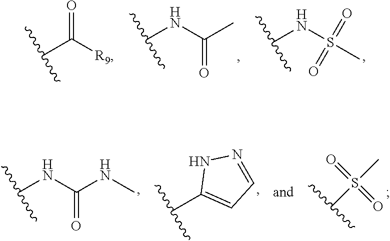

wherein R₈ is selected from cycloalkyl, heterocycloalkyl, wherein R₉ is selected from H, alkyl, OH, NH₂, and alkoxy;
wherein A′ may be optionally substituted with 1, 2, or 3 further substituents R₁₀, R₁₁, and R₁₇, wherein R₁₀, R₁₁, and R₁₇, are independently selected from alkyl, cycloalkyl, alkoxy, OH, oxo, CF₃, and halo, or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O.
R₂ can be selected from —C(═O)—N(R₄)(R₅), —C(═O)—O—R₆,—CH₂—O—CH₃, and —CN, wherein:
R₄ is H or small alkyl, and
R₅ is selected from alkyl, —(CH₂)₀₋₃-cycloalkyl, —(CH₂)₀₋₃-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, and —(C(H)(heteroaryl))-alkyl;
or R₄ and R₅ together with the nitrogen atom to which they are attached can form a carbon-containing 5-, or 6-membered saturated heterocyclic ring, optionally containing an additional heteroatom selected from N, and O, which may be optionally mono- or di-substituted with substituents selected from oxo, alkyl, alkoxy, OH, halo and CF₃; and R₆ is H, small alkyl, and —(CH₂)-phenyl; and
R₁ can be:

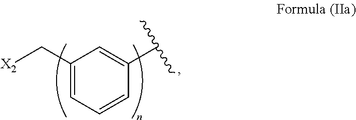

wherein:

nis 1;
X₂ is a non-aromatic, carbon-containing mono- or bi-cyclic ring containing 5, 6, or 7 ring members, wherein at least one ring member is independently selected from N, S, and O;
wherein X₂ may be optionally substituted with 1 or 2 substituents R₁₈ and R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O; or
(ii) Formula (III) comprising an aromatic ring A fused to a non-aromatic ring A′,

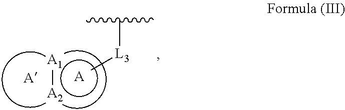

wherein:
L₃ is a single bond;
A₁ and Az are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R₇ is selected from alkyl, alkoxy, and —(CH₂)₀₋₃-R₈,

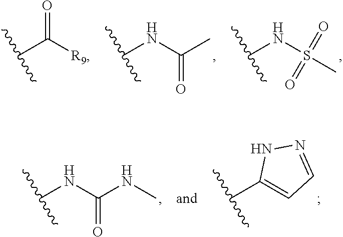

wherein R₈ is selected from cycloalkyl, heterocycloalkyl, wherein R₉ is selected from H, alkyl, OH, NH₂, and alkoxy;
wherein A′ may be optionally substituted with 1 or 2 further substituents R₁₀ and R₁₁, wherein R₁₀ and R₁₁ are independently selected from alkyl, cycloalkyl, alkoxy, OH, oxo, CF₃, and halo, or wherein A′ may be optionally substituted with 1 further substituent R₂₄ selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O.
As noted above, R₃ is phenyl, or a 5- or 6-membered carbon-containing aromatic ring containing one, two or three ring members that are selected from N, S, and O, wherein R₃ is substituted with one, two, or three substituents independently selected from small alkyl^{b}, small alkoxy, cyclopropyl, OH, NH₂, CF₃, halo, —C(═O)—NH₂, —C(═O)—N(H)(small alkyl), —C(═O)—N(small alkyl)(small alkyl), and CN.
R₃ can be phenyl, or a 5- or 6-membered carbon-containing aromatic ring containing one, two or three ring members that are selected from N, S, and O, wherein R₃ is substituted with one or two substituents independently selected from small alkyl^{b}, small alkoxy, cyclopropyl, OH, NH₂, CF₃, halo, —C(═O)—NH₂, —C(═O)—N(H)(small alkyl), —C(═O)—N(small alkyl)(small alkyl), and CN.
R₃ can be phenyl, or a 5- or 6-membered carbon-containing aromatic ring containing one, two or three ring members that are selected from N, S, and O, wherein R₃ is substituted with one or two substituents independently selected from small alkyl, small alkoxy, cyclopropyl, OH, CF₃, halo, —C(═O)—NH₂, —C(═O)—N(H)(small alkyl), —C(═O)—N(small alkyl)(small alkyl), and CN.
R₃ can be selected from substituted phenyl, substituted pyridyl, and substituted thiophene.
R₃ can be substituted phenyl or substituted pyridyl.
R₃ can be substituted phenyl.

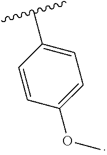 [SMILES: *c1ccc(OC)cc1]

In some embodiments, R₃ is not In some embodiments, when R₃ is substituted with one substituent only, R₃ is not substituted with a 4-methoxy group. In some embodiments, R₃ is not substituted with a 4-methoxy group. In some embodiments, when R₃ is a substituted six-membered ring, R₃ is not substituted with a methoxy group at the para-position to the attachment to B₆. In some embodiments, R₃ is not substituted with a 4-alkoxy group. In some embodiments, when R₃ is a substituted six-membered ring, R₃ is not substituted with an alkoxy group at the para-position to the attachment to B₆. In some embodiments, R₃ is not substituted with any methoxy (—OCH₃) groups.

When R₃ is substituted with three substituents, at least one substituent can be halo. Preferably, when R₃ is substituted with three substituents, at least one substituent can be F or C_{1.}
R₃ can be substituted with one, two, or three substituents independently selected from small alkyl^{b}, small alkoxy, cyclopropyl, OH, NH₂, CF₃, halo, —C(═O)—NH₂, —C(═O)—N(H)(small alkyl), —C(═O)—N(small alkyl)(small alkyl), and CN, wherein R₃ is not substituted with any methoxy (—OCH₃) groups.
R₃ can be substituted with one or two substituents independently selected from small alkyl^{b}, small alkoxy, cyclopropyl, OH, NH₂, CF₃, halo, —C(═O)—NH₂, —C(═O)—N(H)(small alkyl), —C(═O)—N(small alkyl)(small alkyl), and CN, wherein R₃ is not substituted with any methoxy (—OCH₃) groups.
R₃ can be substituted with one or two substituents independently selected from small alkyl^{b}, small alkoxy, cyclopropyl, OH, CF₃, halo, —C(═O)—NH₂, —C(═O)—N(H)(small alkyl), —C(═O)—N(small alkyl)(small alkyl), and CN, wherein R₃ is not substituted with any methoxy (—OCH₃) groups.
R₃ can be substituted with one or two substituents independently selected from small alkyl, small alkoxy, cyclopropyl, OH, CF₃, halo, —C(═O)—NH₂, —C(═O)—N(H)(small alkyl), —C(═O)—N(small alkyl)(small alkyl), and CN, wherein R₃ is not substituted with any methoxy (—OCH₃) groups.
R₃ can be substituted with one, two or three substituents independently selected from small alkyl^{c}, small alkoxy, cyclopropyl, OH, NH₂, CF₃, halo, —C(═O)—NH₂, —C(═O)—N(H)(small alkyl), —C(═O)—N(small alkyl)(small alkyl), and CN, wherein R₃ is not substituted with any methoxy (—OCH₃) groups.
R₃ can be substituted with one or two substituents independently selected from small alkyl^{c}, small alkoxy, cyclopropyl, OH, NH₂, CF₃, halo, —C(═O)—NH₂, —C(═O)—N(H)(small alkyl), —C(═O)—N(small alkyl)(small alkyl), and CN, wherein R₃ is not substituted with any methoxy (—OCH₃) groups.
R₃ can be substituted with one or two substituents independently selected from small alkyl^{c}, small alkoxy, cyclopropyl, OH, CF₃, halo, —C(═O)—NH₂, —C(═O)—N(H)(small alkyl), —C(═O)—N(small alkyl)(small alkyl), and CN, wherein R₃ is not substituted with any methoxy (—OCH₃) groups.
R₃ can be substituted with one or two substituents independently selected from small alkyl^{a}, small alkoxy, cyclopropyl, OH, CF₃, halo, —C(═O)—NH₂, —C(═O)—N(H)(small alkyl), —C(═O)—N(small alkyl)(small alkyl), and CN, wherein R₃ is not substituted with any methoxy (—OCH₃) groups.
R₃ can be substituted with one or two substituents independently selected from small alkyl^{b}, small alkoxy, cyclopropyl, OH, NH₂, CF₃, halo, —C(═O)—NH₂, —C(═O)—N(H)(small alkyl), and CN.
R₃ can be substituted with one or two substituents independently selected from small alkyl^{b}, small alkoxy, cyclopropyl, OH, CF₃, halo, —C(═O)—NH₂, —C(═O)—N(H)(small alkyl), and CN.
R₃ can be substituted with one or two substituents independently selected from small alkyl, small alkoxy, cyclopropyl, OH, CF₃, halo, —C(═O)—NH₂, —C(═O)—N(H)(small alkyl), and CN.
R₃ can be substituted with one or two substituents independently selected from small alkyl^{c}, small alkoxy, cyclopropyl, OH, NH₂, CF₃, halo, —C(═O)—NH₂, —C(═O)—N(H)(small alkyl), and CN.
R₃ can be substituted with one or two substituents independently selected from small alkyl^{c}, small alkoxy, cyclopropyl, OH, CF₃, halo, —C(═O)—NH₂, —C(═O)—N(H)(small alkyl), and CN.
R₃ can be substituted with one or two substituents independently selected from small alkyl^{a}, small alkoxy, cyclopropyl, OH, CF₃, halo, —C(═O)—NH₂, —C(═O)—N(H)(small alkyl), and CN.
R₃ can be substituted with one or two substituents independently selected from small alkyl^{b}, cyclopropyl, OH, NH₂, CF₃, halo, —C(═O)—NH₂, —C(═O)—N(H)(small alkyl), —C(═O)—N(small alkyl)(small alkyl), and CN.
R₃ can be substituted with one or two substituents independently selected from small alkyl^{b}, cyclopropyl,

OH, CF₃, halo, —C(═O)—NH₂, —C(═O)—N(H)(small alkyl), —C(═O)—N(small alkyl)(small alkyl), and CN.
R₃ can be substituted with one or two substituents independently selected from small alkyl, cyclopropyl, OH, CF₃, halo, —C(═O)—NH₂, —C(═O)—N(H)(small alkyl), —C(═O)—N(small alkyl)(small alkyl), and CN.
R₃ can be substituted with one or two substituents independently selected from small alkyl^{c}, cyclopropyl, OH, NH₂, CF₃, halo, —C(═O)—NH₂, —C(═O)—N(H)(small alkyl), —C(═O)—N(small alkyl)(small alkyl), and CN.
R₃ can be substituted with one or two substituents independently selected from small alkyl^{c}, cyclopropyl, OH, CF₃, halo, —C(═O)—NH₂, —C(═O)—N(H)(small alkyl), —C(═O)—N(small alkyl)(small alkyl), and CN.
R₃ can be substituted with one or two substituents independently selected from small alkyl^{a}, cyclopropyl, OH, CF₃, halo, —C(═O)—NH₂, —C(═O)—N(H)(small alkyl), —C(═O)—N(small alkyl)(small alkyl), and CN.
R₃ can be substituted with one or two substituents independently selected from small alkyl^{b}, small alkoxy,

OH, NH₂, CF₃, halo, —C(═O)—NH₂, and CN.
R₃ can be substituted with one or two substituents independently selected from small alkyl, small alkoxy, OH, NH₂, CF₃, halo, —C(═O)—NH₂, and CN.
R₃ can be substituted with one or two substituents independently selected from small alkyl, small alkoxy, CF₃, halo, —C(═O)—NH₂, and CN.
R₃ can be substituted with one or two substituents independently selected from small alkyl^{c}, small alkoxy, OH, NH₂, CF₃, halo, —C(═O)—NH₂, and CN.
R₃ can be substituted with one or two substituents independently selected from small alkyl^{c}, small alkoxy, OH, CF₃, halo, —C(═O)—NH₂, and CN.
R₃ can be substituted with one or two substituents independently selected from small alkyl^{a}, small alkoxy, CF₃, halo, —C(═O)—NH₂, and CN.

Preferably, R₃ is phenyl or pyridyl substituted with one or two substituents independently selected from small alkyl^{b}, small alkoxy, OH, NH₂, CF₃, halo, —C(═O)—NH₂, and CN.
R₃ can be phenyl substituted with one or two substituents independently selected from small alkyl^{b}, small alkoxy, OH, NH₂, CF₃, halo, —C(═O)—NH₂, and CN.
R₃ can be phenyl or pyridyl substituted with one or two substituents independently selected from small alkyl^{b}, small alkoxy, OH, CF₃, halo, —C(═O)—NH₂, and CN.
R₃ can be phenyl substituted with one or two substituents independently selected from small alkyl^{b}, small alkoxy, OH, CF₃, halo, —C(═O)—NH₂, and CN.
R₃ can be phenyl substituted with one or two substituents independently selected from small alkyl, small alkoxy, CF₃, halo, —C(═O)—NH₂, and CN.
R₃ can be phenyl substituted with one or two substituents independently selected from small alkyl^{c}, small alkoxy, CF₃, halo, —C(═O)—NH₂, and CN.
R₃ can be phenyl substituted with one or two substituents independently selected from small alkyl^{c}, small alkoxy, OH, NH₂, CF₃, halo, —C(═O)—NH₂, and CN.
R₃ can be phenyl substituted with one or two substituents independently selected from small alkyl^{a}, small alkoxy, CF₃, halo, —C(═O)—NH₂, and CN.
R₃ can be substituted with one or two substituents independently selected from small alkyl^{b}, small alkoxy, CF₃, OH, NH₂, halo, —C(═O)—NH₂, and CN, wherein R₃ is not substituted with a 4-methoxy (—OCH₃) group.
R₃ can be substituted with one or two substituents independently selected from small alkyl^{b}, small alkoxy,

OH, CF₃, halo, —C(═O)—NH₂, and CN, wherein R₃ is not substituted with a 4-methoxy (—OCH₃) group.
R₃ can be substituted with one or two substituents independently selected from small alkyl, small alkoxy, CF₃, halo, —C(═O)—NH₂, and CN, wherein R₃ is not substituted with a 4-methoxy (—OCH₃) group.
R₃ can be substituted with one or two substituents independently selected from small alkyl^{c}, small alkoxy, OH, NH₂, CF₃, halo, —C(═O)—NH₂, and CN, wherein R₃ is not substituted with a 4-methoxy (—OCH₃) group.
R₃ can be substituted with one or two substituents independently selected from small alkyl^{c}, small alkoxy,

OH, CF₃, halo, —C(═O)—NH₂, and CN, wherein R₃ is not substituted with a 4-methoxy (—OCH₃) group.
R₃ can be substituted with one or two substituents independently selected from small alkyl^{a}, small alkoxy, CF₃, halo, —C(═O)—NH₂, and CN, wherein R₃ is not substituted with a 4-methoxy (—OCH₃) group.
R₃ can be substituted with one or two substituents independently selected from —CH₃,—CH₂—OH, —CH₂—NH₂, —CH₂—O—CH₃,—OCH₃, OH, NH₂, CF₃, halo, —C(═O)—NH₂, and CN.
R₃ can be substituted with one or two substituents independently selected from —CH₃,—CH₂—OH, —CH₂—NH₂, —CH₂—O—CH₃,—OCH₃, OH, CF₃, halo, —C(═O)—NH₂, and CN.
R₃ can be substituted with one or two substituents independently selected from —CH₃,—CH₂—NH₂,—OCH₃, CF₃, halo, —C(═O)—NH₂, and CN.
R₃ can be substituted with one or two substituents independently selected from —CH₃,—CH₂—OH, —CH₂—O—CH₃,—OCH₃, OH, CF₃, halo, —C(═O)—NH₂, and CN.
R₃ can be substituted with one or two substituents independently selected from —CH₃,—OCH₃, CF₃, halo, —C(═O)—NH₂, and CN.
R₃ can be substituted with one or two substituents independently selected from —CH₃,—CH₂—OH, —CH₂—NH₂, —CH₂—O—CH₃, OH, CF₃, halo, —C(═O)—NH₂, and CN.
R₃ can be substituted with one or two substituents independently selected from —CH₃,—CH₂—NH₂, CF₃, halo, —C(═O)—NH₂, and CN.
R₃ can be substituted with one or two substituents independently selected from —CH₃,—CH₂—OH, —CH₂—O—CH₃, OH, CF₃, halo, —C(═O)—NH₂, and CN.
R₃ can be substituted with one or two substituents independently selected from —CH_{3,}

CF₃, halo, —C(═O)—NH₂, and CN.

When R₃ is substituted with two substituents, R₃ can be substituted with a 3-chloro group. When R₃ is a six-membered ring substituted with two substituents, R₃ can be substituted with a chlorine substituent at the meta-position to the attachment to B_{6.}

When R₃ is a six-membered ring substituted with three substituents, R₃ can be substituted with a chlorine substituent at the meta-position to the attachment to B_{6.}
R₃ can be substituted with one or two substituents independently selected from —CH₃,—CH₂—OH, —CH₂—NH₂, —CH₂—O—CH₃, OH, CF₃, halo, and CN.
R₃ can be substituted with one or two substituents independently selected from —CH₃,—CH₂—NH₂, CF₃, halo, and CN.
R₃ can be substituted with one or two substituents independently selected from —CH₃,—CH₂—OH, —CH₂—O—CH₃, OH, CF₃, halo, and CN.
R₃ can be substituted with one or two substituents independently selected from —CH₃, CF₃, halo, and CN.
R₃ can be substituted with a small alkyl^{b} and optionally a further substituent selected from small alkyl^{b}, small alkoxy, cyclopropyl, OH, NH₂, CF₃, halo, —C(═O)—NH₂, —C(═O)—N(H)(small alkyl), —C(═O)—N(small alkyl)(small alkyl), and CN.
R₃ can be substituted with a small alkyl and optionally a further substituent selected from small alkyl, small alkoxy, cyclopropyl, OH, CF₃, halo, —C(═O)—NH₂, —C(═O)—N(H)(small alkyl), —C(═O)—N(small alkyl)(small alkyl), and CN.
R₃ can be substituted with a (C₁-C₃) alkyl substituted with NH₂, and wherein R₃ is optionally substituted with a further substituent selected from small alkyl^{b}, small alkoxy, cyclopropyl, OH, NH₂, CF₃, halo, —C(═O)—NH₂, —C(═O)—N(H)(small alkyl), —C(═O)—N(small alkyl)(small alkyl), and CN.
R₃ can be substituted with a (C₁-C₃) alkyl substituted with NH₂, and wherein R₃ is optionally substituted with a further substituent selected from small alkyl, small alkoxy, cyclopropyl, OH, CF₃, halo, —C(═O)—NH₂, —C(═O)—N(H)(small alkyl), —C(═O)—N(small alkyl)(small alkyl), and CN.
R₃ can be substituted with —CH₂NH₂, and optionally a further substituent selected from small alkyl^{b}, small alkoxy, cyclopropyl, OH, NH₂, CF₃, halo, —C(═O)—NH₂, —C(═O)—N(H)(small alkyl), —C(═O)—N(small alkyl)(small alkyl), and CN.
R₃ can be substituted with —CH₂NH₂, and optionally a further substituent selected from small alkyl, small alkoxy, cyclopropyl, OH, CF₃, halo, —C(═O)—NH₂, —C(═O)—N(H)(small alkyl), —C(═O)—N(small alkyl)(small alkyl), and CN.
R₃ can be substituted with two substituents, wherein one of the substituents is —CH₂NH_{2.}
R₃ can be phenyl or pyridyl substituted with one or two halo substituents. R₃ can be phenyl substituted with one or two halo substituents. R₃ can be phenyl or pyridyl substituted with one or two substituents selected from F and C₁. R₃ can be phenyl substituted with one or two substituents selected from F and C₁.

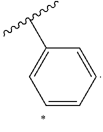 [SMILES: *c1ccccc1]

R₃ can be substituted with one substituent at the meta-position, labelled with a (*) R₃ can be substituted with one substituent at the meta-position, labelled with a (*), and one substituent at the

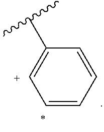 [SMILES: *C1=[C+]C=CC=C1]

ortho-position, labelled with a (+)
R₃ can be substituted with one substituent at the meta

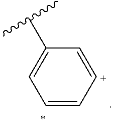 [SMILES: *C1=CC=C[C+]=C1]

position, labelled with a (*), and one substituent at the meta-position, labelled with a (+)

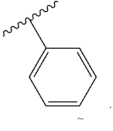 [SMILES: *c1ccccc1]

R₃ can be unsubstituted at the para-position, labelled with a (˜)

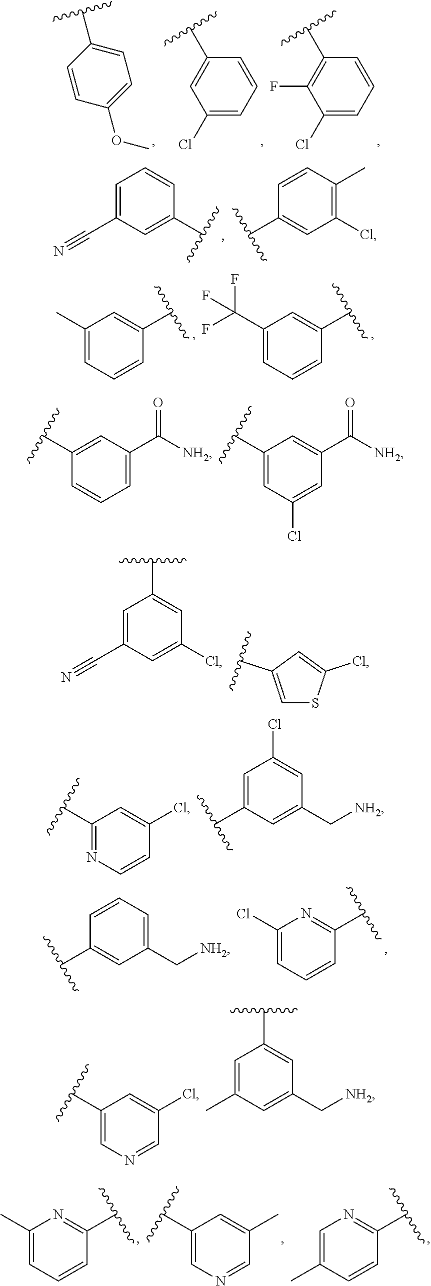

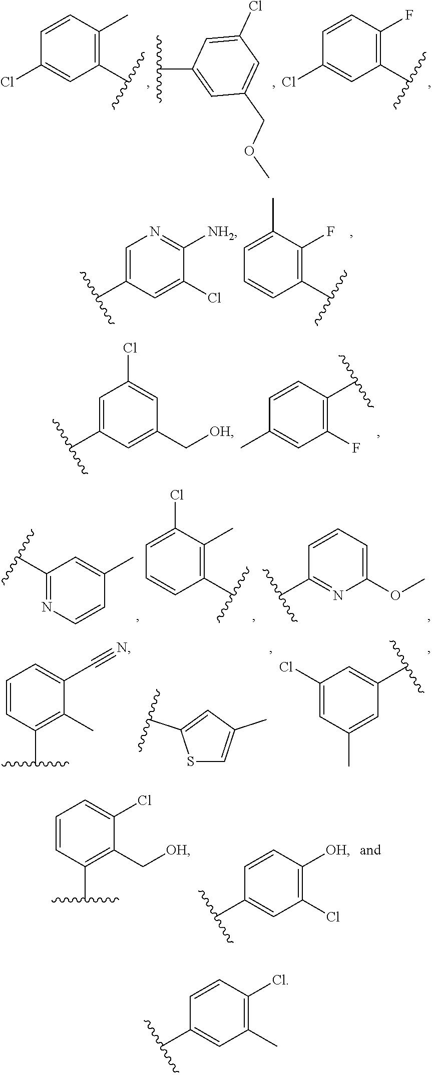

R₃ can be selected from:

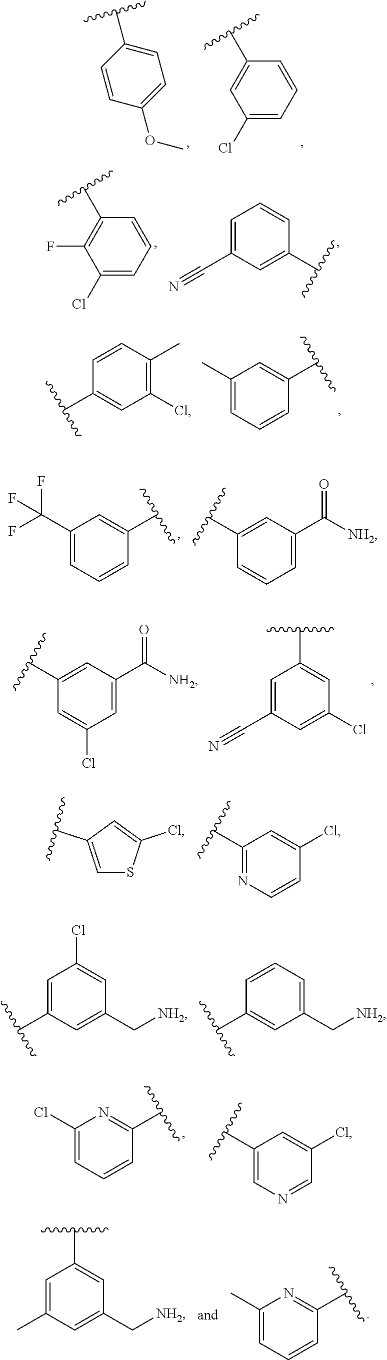

R₃ can be selected from:

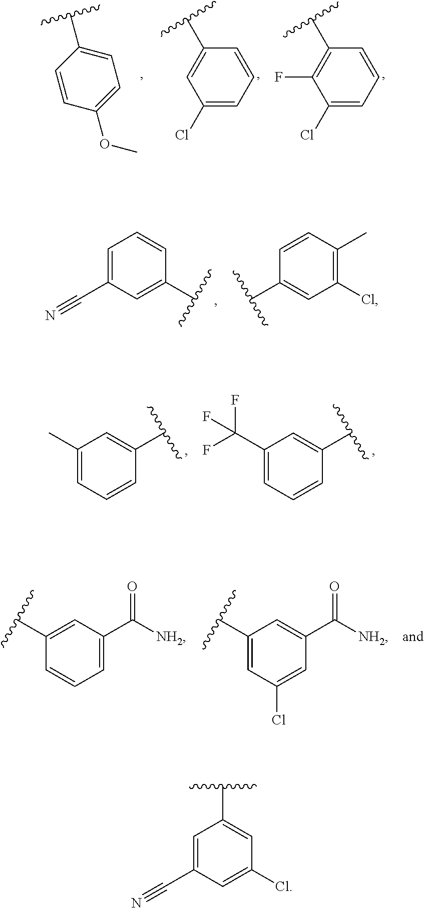

R₃ can be selected from:

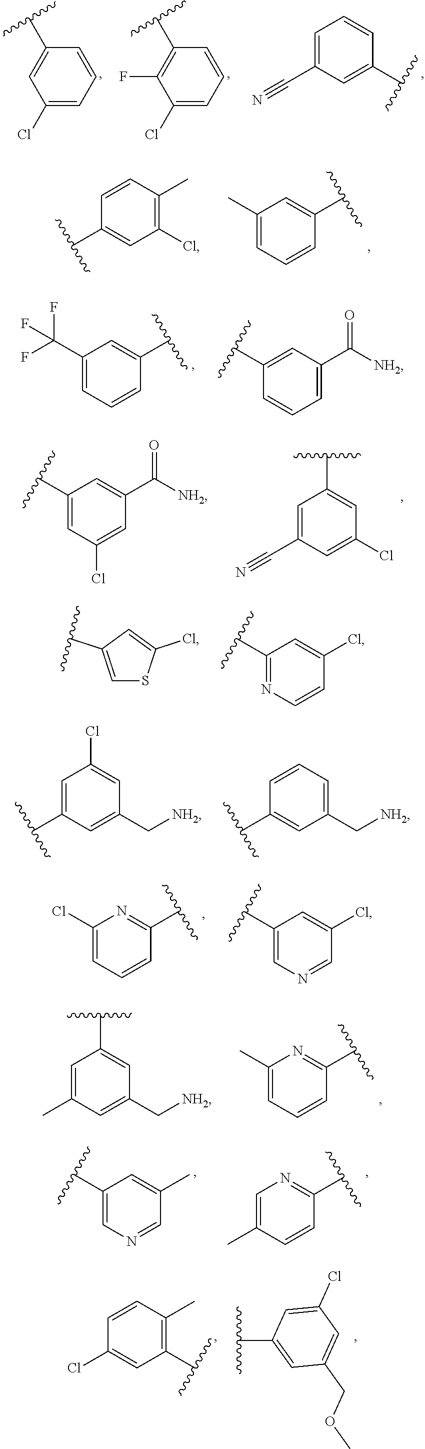

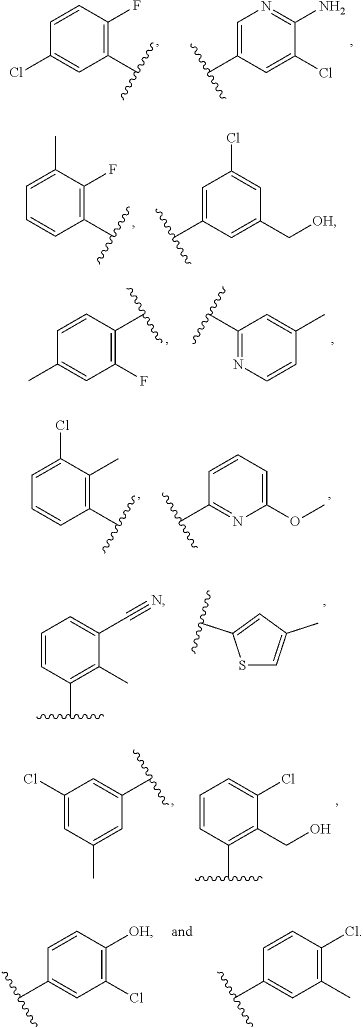

R₃ can be selected from:

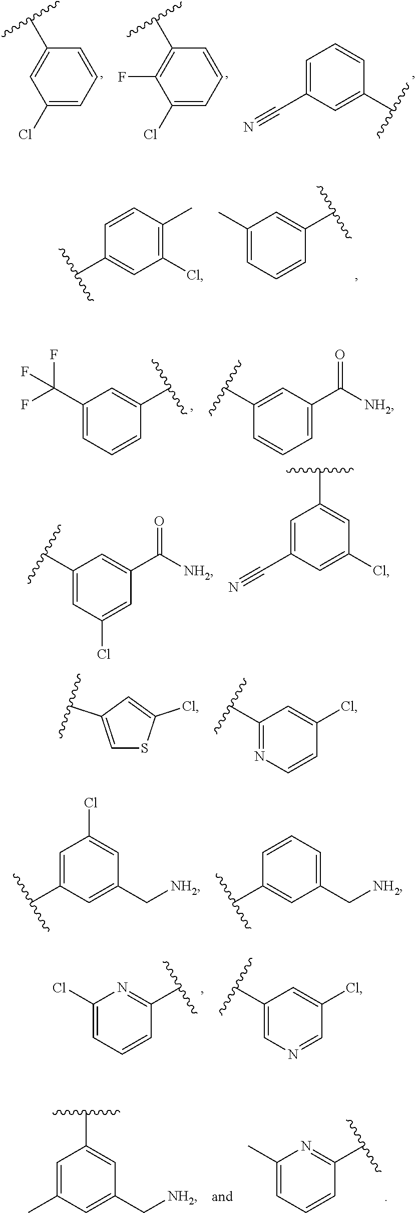

R₃ can be selected from:

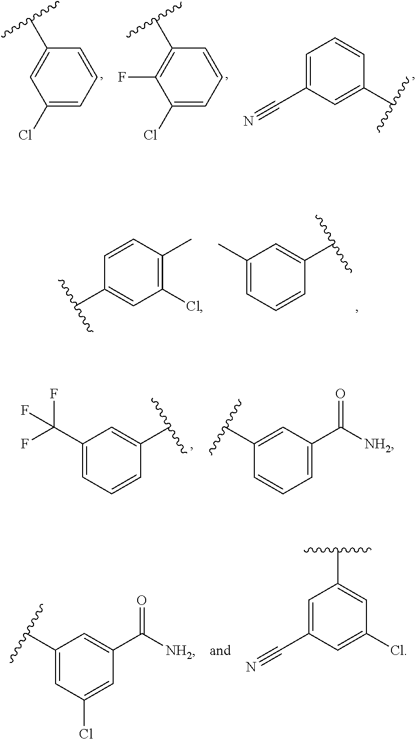

R₃ can be selected from:

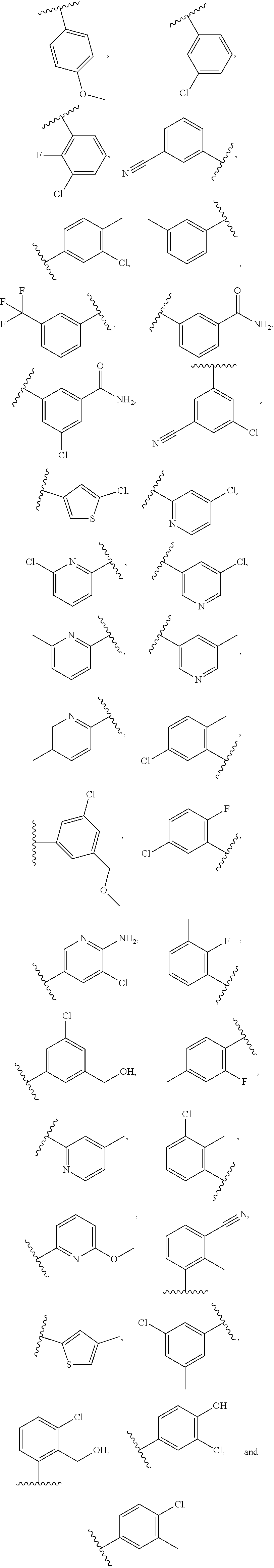

R₃ can be selected from:

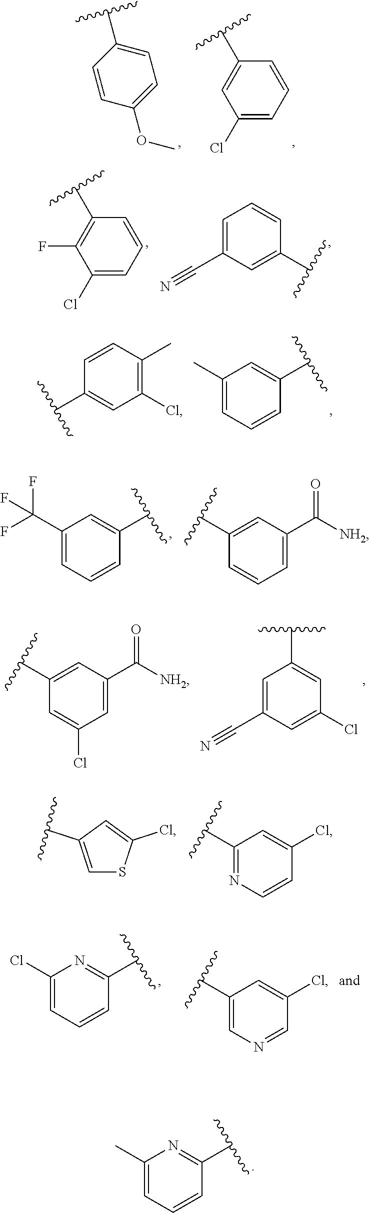

R₃ can be selected from:

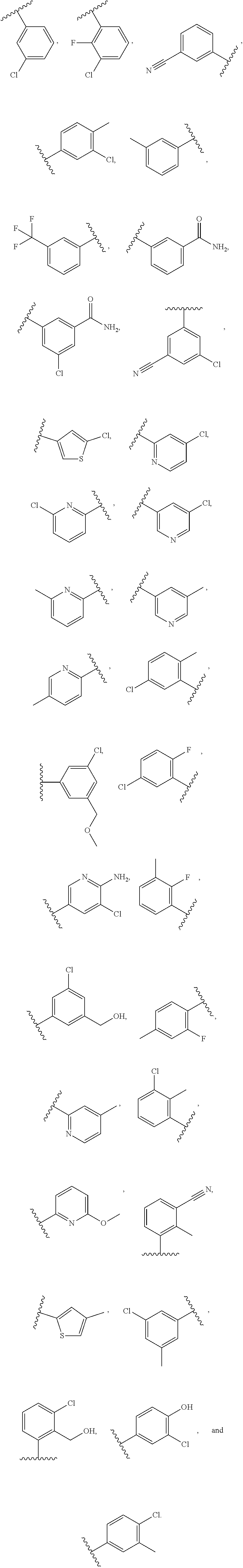

R₃ can be selected from:
R₃ can be selected from:

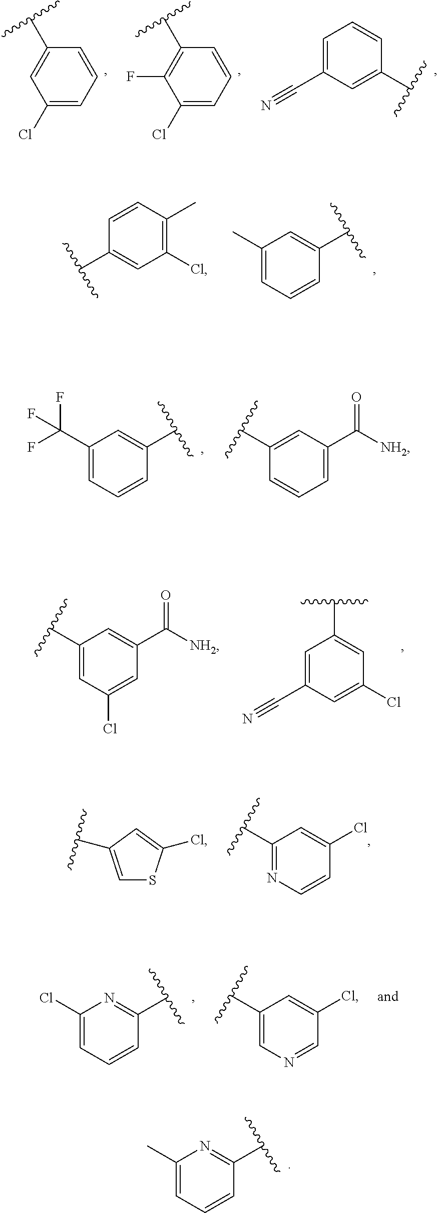

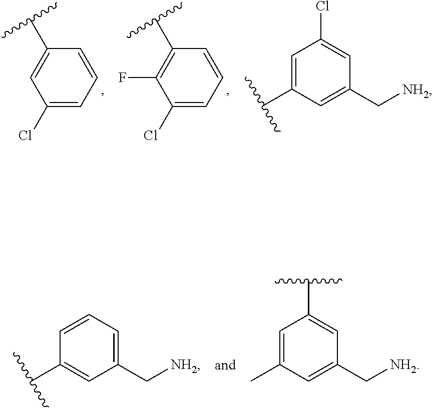

R₃ can be selected from:

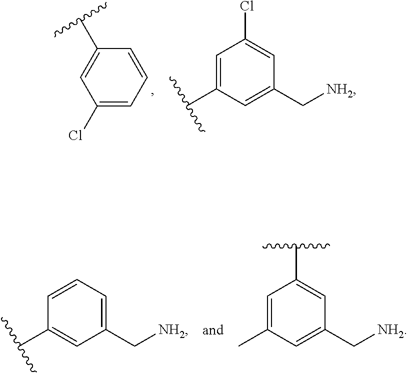

R₃ can be selected from:

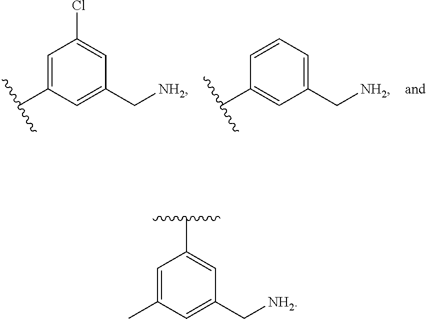

R₃ can be selected from:

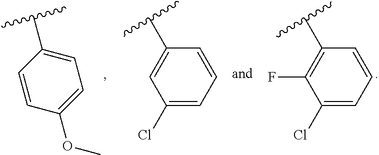

R₃ can be selected from: Preferably, R₃ can be selected from:

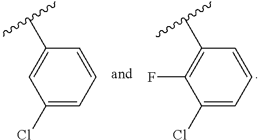 [SMILES: *c1cccc(Cl)c1 ; *c1cccc(Cl)c1F]

R₃ can be

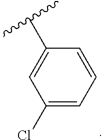 [SMILES: *c1cccc(Cl)c1]

B₁ can be a carbon centre and B₂ can be a carbon centre, wherein B₃ is a single bond between B₂ and the nitrogen atom to which R₁ is attached and B₁ is connected to B₂ via a single bond, or B₁ can be a nitrogen centre and B₂ can be a carbon centre wherein B₁ is connected to B₂ via a double bond; and R₃ can be substituted with one or two substituents independently selected from small alkyl^{b}, small alkoxy, OH, NH₂, CF₃, halo, —C(═O)—NH₂, and CN.
B₄ can be N(R₂), B₅ can be N, and B₆ can be C(R₃), wherein B₄ is connected to B₅ via a single bond and B₅ is connected to B₆ via a double bond, or B₄ can be C(R₂), B₅ can be N, and B₆ can be N(R₃), wherein B₄ is connected to B₅ via a double bond and B₅ is connected to B₆ via a single bond; and R₃ can be substituted with one or two substituents independently selected from small alkyl^{b}, small alkoxy, OH, NH₂, CF₃, halo, —C(═O)—NH₂, and CN.
B₁ can be a carbon centre and B₂ can be a carbon centre, wherein B₃ is a single bond between B₂ and the nitrogen atom to which R₁ is attached and B₁ is connected to B₂ via a single bond, or B₁ can be a nitrogen centre and B₂ can be a carbon centre wherein B₁ is connected to B₂ via a double bond; B₄ can be N(R₂), B₅ can be N, and B₆ can be C(R₃), wherein B₄ is connected to B₅ via a single bond and B₅ is connected to B₆ via a double bond, or B₄ can be C(R₂), B₅ can be N, and B₆ can be N(R₃), wherein B₄ is connected to B₅ via a double bond and B₅ is connected to B₆ via a single bond; and R₃ can be substituted with one or two substituents independently selected from small alkyl^{b}, small alkoxy, OH, NH₂, CF₃, halo, —C(═O)—NH₂, and CN.
B₁ can be a carbon centre and B₂ can be a carbon centre, wherein B₃ is a single bond between B₂ and the nitrogen atom to which R₁ is attached and B₁ is connected to B₂ via a single bond, or B₁ can be a nitrogen centre and B₂ can be a carbon centre wherein B₁ is connected to B₂ via a double bond; and R₂ can be from —C(═O)—N(R₄)(R₅), —C(═O)—O—R₆,—CH₂—NH—C(═O) CH₃,—CH₂—NH—S(═O)₂R₃₅, selected

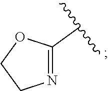 [SMILES: *C1=NCCO1]

—CH₂—O—R₁₆,—CH₂—OH, —CN, and and R₃ can be substituted with one or two substituents independently selected from small alkyl^{b}, small alkoxy, OH, NH₂, CF₃, halo, —C(═O)—NH₂, and CN.
B₄ can be N(R₂), B₅ can be N, and B₆ can be C(R₃), wherein B₄ is connected to B₅ via a single bond and B₅ is connected to B₆ via a double bond, or B₄ can be C(R₂), B₅ can be N, and B₆ can be N(R₃), wherein B₄ is connected to B₅ via a double bond and B₅ is connected to B₆ via a single bond; R₂ can be selected from

C(═O)—N(R₄)(R₅), —C(═O)—O—R₆,—CH₂—NH—C(═O) CH₃,—CH₂—NH—S(═O)₂R₃₅,—CH₂—O—R₁₆,—CH₂—OH, —CN, and

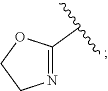 [SMILES: *C1=NCCO1]

and R₃ can be substituted with one or two substituents independently selected from small alkyl^{b}, small alkoxy, OH, NH₂, CF₃, halo, —C(═O)—NH₂, and CN.
B₁ can be a carbon centre and B₂ can be a carbon centre, wherein B₃ is a single bond between B₂ and the nitrogen atom to which R₁ is attached and B₁ is connected to B₂ via a single bond, or B₁ can be a nitrogen centre and B₂ can be a carbon centre wherein B₁ is connected to B₂ via a double bond; B₄ can be N(R₂), B₅ can be N, and B₆ can be C(R₃), wherein B₄ is connected to B₅ via a single bond and B₅ is connected to B₆ via a double bond, or B₄ can be C(R₂), B₅ can be N, and B₆ can be N(R₃), wherein B₄ is connected to B₅ via a double bond and B₅ is connected to B₆ via a single bond; R₂ can be selected from —C(═O)—N(R₄)(R₅),

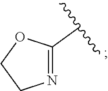 [SMILES: *C1=NCCO1]

—C(═O)—O—R₆,—CH₂—NH—S(═O)₂R₃₅,—CH₂—NH—C(═O) CH₃,—CH₂—O—R₁₆,—CH₂—OH, —CN, and and R₃ can be substituted with one or two substituents independently selected from small alkyl^{b}, small alkoxy, OH, NH₂, CF₃, halo, —C(═O)—NH₂, and CN.
R₂ can be selected from —C(═O)—N(R₄)(R₅), —C(═O)—O—R₆,—CH₂—NH—C(═O) CH₃,—CH₂—NH—S(═O)₂R₃₅,—CH₂—O—R₁₆,

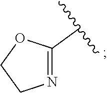 [SMILES: *C1=NCCO1]

—CH₂—OH, —CN, and and R₃ can be substituted with one or two substituents independently selected from small alkyl^{b}, small alkoxy, OH, NH₂, CF₃, halo, —C(═O)—NH₂, and CN.
B₁ can be a carbon centre and B₂ can be a carbon centre, wherein B₃ is a single bond between B₂ and the nitrogen atom to which R₁ is attached and B₁ is connected to B₂ via a single bond, or B₁ can be a nitrogen centre and B₂ can be a carbon centre wherein B₁ is connected to B₂ via a double bond; and R₂ can be selected from —C(═O)—N(R₄)(R₅), —C(═O)—O—R₆,—CH₂—NH—C(═O) CH₃,—CH₂—NH—S(═O)₂R₃₅,—CH₂—O—R₁₆,—CH₂—OH, —

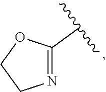 [SMILES: *C1=NCCO1]

CN, and wherein:
R₄ is H or small alkyl, and
R₅ is selected from alkyl, OH, —S(═O)₂-(small alkyl), —(CH₂)₁₋₃—(C═O)—OH, —(CH₂)₀₋₃-cycloalkyl^{b}, —(CH₂)₀₋₃-heterocycloalkyl, —(CH(alkyl))-heterocycloalkyl, —(CH₂)₀₋₃-polycycloalkyl, —(CH₂)₀₋₃-aryl, —(C(H)(phenyl)-alkyl, —(CH₂)₀₋₃heteroaryl, —(C(H)(heteroaryl)-alkyl, —(C(H)(alkyl)- (C═O)—OH, and —(C(H)(heteroaryl))—(C═O)—OR_{28;}
or R₄ and R₅ together with the nitrogen atom to which they are attached can form a carbon-containing 4-, 5-, or 6-membered saturated heterocyclic ring, optionally containing an additional heteroatom selected from N, and O, which may be optionally mono- or di-substituted with substituents selected from oxo, alkyl, alkoxy, OH, halo and CF₃; R₆ is H, small alkyl, and —(CH₂)-phenyl;
R₁₆ is selected from H, small alkyl, —(CH₂)—(C═O)—OH, heterocycloalkyl, and —(CH₂)-heteroaryl; and R₃₅ is selected from H, small alkyl, and cyclopropyl; and
R₃ can be substituted with one or two substituents independently selected from small alkyl^{b}, small alkoxy, OH, NH₂, CF₃, halo, —C(═O)—NH₂, and CN.
B₄ can be N(R₂), B₅ can be N, and B₆ can be C(R₃), wherein B₄ is connected to B₅ via a single bond and B₅ is connected to B₆ via a double bond, or B₄ can be C(R₂), B₅ can be N, and B₆ can be N(R₃), wherein B₄ is connected to B₅ via a double bond and B₅ is connected to B₆ via a single bond; R₂ can be selected from-C(═O)—N(R₄)(R₅), —C(═O)—O—R₆,—CH₂—NH—C(═O) CH₃,—CH₂—NH—S(═O)₂R₃₅,—CH₂—O—R₁₆,—CH₂—OH, —CN, and

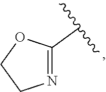

wherein:
R₄ is H or small alkyl, and
R₅ is selected from alkyl, OH, —S(═O)₂-(small alkyl), —(CH₂)₁₋₃—(C═O)—OH, —(CH₂)₀₋₃-cycloalkyl^{b}, —(CH₂)₀₋₃-heterocycloalkyl, —(CH(alkyl)-heterocycloalkyl, —(CH₂)₀₋₃-polycycloalkyl, —(CH₂)₀₋₃-aryl, —(C(H)(phenyl)-alkyl, —(CH₂)₀₋₃heteroaryl, —(C(H)(heteroaryl)-alkyl, —(C(H)(alkyl)—(C═O)—OH, and —(C(H)(heteroaryl))—(C═O)—OR_{28;}
or R₄ and R₅ together with the nitrogen atom to which they are attached can form a carbon-containing 4-, 5-, or 6-membered saturated heterocyclic ring, optionally containing an additional heteroatom selected from N, and O, which may be optionally mono- or di-substituted with substituents selected from oxo, alkyl, alkoxy, OH, halo and CF_{3;}
R₆ is H, small alkyl, and —(CH₂)-phenyl;
R₁₆ is selected from H, small alkyl, —(CH₂)—(C═O)—OH, heterocycloalkyl, and —(CH₂)-heteroaryl; and R₃₅ is selected from H, small alkyl, and cyclopropyl; and
R₃ can be substituted with one or two substituents independently selected from small alkyl^{b}, small alkoxy, OH, NH₂, CF₃, halo, —C(═O)—NH₂, and CN.
B₁ can be a carbon centre and B₂ can be a carbon centre, wherein B₃ is a single bond between B₂ and the nitrogen atom to which R₁ is attached and B₁ is connected to B₂ via a single bond, or B₁ can be a nitrogen centre and B₂ can be a carbon centre wherein B₁ is connected to B₂ via a double bond; B₄ can be N(R₂), B₅ can be N, and B₆ can be C(R₃), wherein B₄ is connected to B₅ via a single bond and B₅ is connected to B₆ via a double bond, or B₄ can be C(R₂), B₅ can be N, and B₆ can be N(R₃), wherein B₄ is connected to B₅ via a double bond and B₅ is connected to B₆ via a single bond; R₂ can be selected from —C(═O)—N(R₄)(R₅),

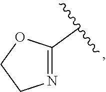 [SMILES: *C1=NCCO1]

—C(═O)—O—R₆,—CH₂—NH—S(═O)₂R₃₅,—CH₂—NH—C(═O) CH₃,—CH₂—O—R₁₆,—CH₂—OH, —CN, and wherein:
R₄ is H or small alkyl, and
R₅ is selected from alkyl, OH, —S(═O)₂-(small alkyl), —(CH₂)₁₋₃—(C═O)—OH, —(CH₂)₀₋₃-cycloalkyl^{b}, —(CH₂)₀₋₃-heterocycloalkyl, —(CH(alkyl))-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, —(C(H)(heteroaryl))-alkyl, —(C(H)(alkyl))—(C═O)—OH, and —(C(H)(heteroaryl))—(C═O)—OR_{28;}
or R₄ and R₅ together with the nitrogen atom to which they are attached can form a carbon-containing 4-, 5-, or 6-membered saturated heterocyclic ring, optionally containing an additional heteroatom selected from N, and O, which may be optionally mono- or di-substituted with substituents selected from oxo, alkyl, alkoxy, OH, halo and CF_{3;}
R₆ is H, small alkyl, and —(CH₂)-phenyl;
R₁₆ is selected from H, small alkyl, —(CH₂)—(C═O)—OH, heterocycloalkyl, and —(CH₂)-heteroaryl; and R₃₅ is selected from H, small alkyl, and cyclopropyl; and
R₃ can be substituted with one or two substituents independently selected from small alkyl^{b}, small alkoxy, OH, NH₂, CF₃, halo, —C(═O)—NH₂, and CN.
R₂ can be selected from —C(═O)—N(R₄)(R₅), —C(═O)—O—R₆,—CH₂—NH—S(═O)₂R₃₅,—CH₂—NH—C(═O) CH₃,—CH₂—O—R₁₆,

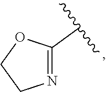 [SMILES: *C1=NCCO1]

—CH₂—OH, —CN, and wherein:
R₄ is H or small alkyl, and
R₅ is selected from alkyl, OH, —S(═O)₂-(small alkyl), —(CH₂)₁₋₃—(C═O)—OH, —(CH₂)₀₋₃-cycloalkyl^{b}, —(CH₂)₀₋₃-heterocycloalkyl, —(CH(alkyl))-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, —(C(H)(heteroaryl))-alkyl, —(C(H)(alkyl))—(C═O)—OH, and —(C(H)(heteroaryl))—(C═O)—OR_{28;}
or R₄ and R₅ together with the nitrogen atom to which they are attached can form a carbon-containing 4-, 5-, or 6-membered saturated heterocyclic ring, optionally containing an additional heteroatom selected from N, and O, which may be optionally mono- or di-substituted with substituents selected from oxo, alkyl, alkoxy, OH, halo and CF₃; R₆ is H, small alkyl, and —(CH₂)-phenyl;
R₁₆ is selected from H, small alkyl, —(CH₂)—(C═O)—OH, heterocycloalkyl, and —(CH₂)-heteroaryl; and
Ras is selected from H, small alkyl, and cyclopropyl; and
R₃ can be substituted with one or two substituents independently selected from small alkyl^{b}, small alkoxy, OH, NH₂, CF₃, halo, —C(═O)—NH₂, and CN.
B₁ can be a carbon centre and B₂ can be a carbon centre, wherein B₃ is a single bond between B₂ and the nitrogen atom to which R₁ is attached and B₁ is connected to B₂ via a single bond, or B₁ can be a nitrogen centre and B₂ can be a carbon centre wherein B₁ is connected to B₂ via a double bond;
R₁ can be:
(i) Formula (IIb),

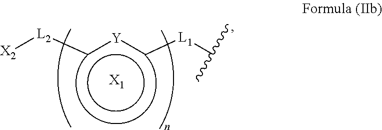

wherein:

n is 1;
L₁ is a linker selected from a single bond and —(CH₂)₁₋₃—;
Y is selected from C, N, O and S; X₁ is phenyl or a 5- or 6-membered carbon-containing aromatic ring containing one, two or three ring members that are selected from N, S, and O;
wherein X₁ may be optionally substituted with 1 or 2 substituents independently selected from alkyl, cycloalkyl, alkoxy, OH, O(cyclopropyl), CF₃, and halo; Ly is a linker selected from a single bond, —(CH₂)₁₋₃—, and —C(═O); and
X₂ is either:
(a) —NR₂OR₂₁, wherein R₂₀ is selected from H, alkyl, and —(CH₂)₀₋₃-R_{22;}
wherein R₂₁ is selected from H, alkyl, and —(CH₂)₀₋₃-R₂₃;

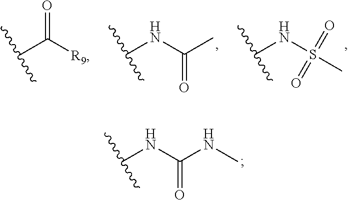

wherein R₂₂ is selected from cycloalkyl, heterocycloalkyl, aryl, heteroaryl,

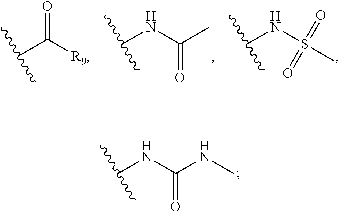

wherein R₂₃ is selected from cycloalkyl, heterocycloalkyl, aryl, heteroaryl, or
(b)₂-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected from alkyl, cycloalkyl, alkoxy, oxo, OH, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O, and X₂ may be further optionally substituted with 1 or 2 substituents R₂₉ and R₃₀, wherein R₂₉ and R₃₀ are independently selected from alkyl, cycloalkyl, alkoxy, OH, oxo, CF₃, and halo; or
(ii) Formula (III) comprising an aromatic ring A fused to a non-aromatic ring A′,

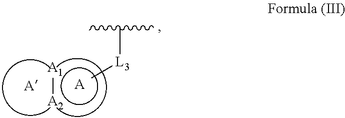

wherein:
L₃ is a single bond;
A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, O(cyclopropyl), CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R₇ is selected from alkyl, alkoxy, and —(CH₂)₀₋₃-R₈,

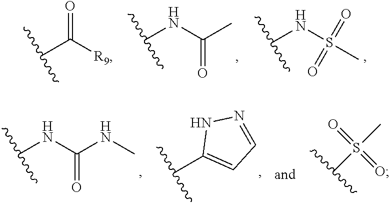

wherein R₈ is selected from cycloalkyl, heterocycloalkyl, wherein R₉ is selected from H, alkyl, OH, N(R₃₁)(R₃₂), and alkoxy;
wherein R₃₁ is H or small alkyl;
wherein R₃₂ is selected from H, small alkyl, and —(CH₂)₀₋₃heteroaryl;
wherein A′ may be optionally substituted with 1, 2, or 3 further substituents R₁₀, R₁₁, and R₁₇, wherein R₁₀, R₁₁, and R₁₇, are independently selected from alkyl, cycloalkyl, alkoxy, OH, oxo, CF₃, and halo, or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O, or wherein A′ may be optionally substituted with 1 or 2 further substituents R₄₃ and R₄₄ independently selected from alkyl, cycloalkyl, alkoxy, OH, oxo, CF₃, and halo, and two different carbon ring members within A′ are connected by a bridging —CH₂-group; and
R₃ can be substituted with one or two substituents independently selected from small alkyl^{b}, small alkoxy,

OH, NH₂, CF₃, halo, —C(═O)—NH₂, and CN.
B₄ can be N(R₂), B₅ can be N, and B₆ can be C(R₃), wherein B₄ is connected to Bs via a single bond and B₅ is connected to B₆ via a double bond, or B₄ can be C(R₂), Bs can be N, and B₆ can be N(R₃), wherein B₄ is connected to B₅ via a double bond and B₅ is connected to B₆ via a single bond;
R₁ can be:
(i) Formula (IIb),

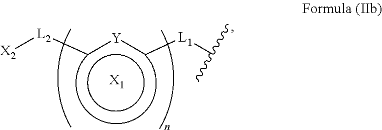

wherein:
n is 1;
L₁ is a linker selected from a single bond and —(CH₂)₁₋₃—;
Y is selected from C, N, O and S;
X₁ is phenyl or a 5- or 6-membered carbon-containing aromatic ring containing one, two or three ring members that are selected from N, S, and O;
wherein X₁ may be optionally substituted with 1 or 2 substituents independently selected from alkyl, cycloalkyl, alkoxy, OH, O(cyclopropyl), CF₃, and halo;
L₂ is a linker selected from a single bond, —(CH₂)₁₋₃—, and —C(═O)—; and
X₂ is either:
(a) —NR₂OR₂₁, wherein R₂₀ is selected from H, alkyl, and —(CH₂)₀₋₃-R_{22;}
wherein R₂₁ is selected from H, alkyl, and —(CH₂)₀₋₃-R₂₃;

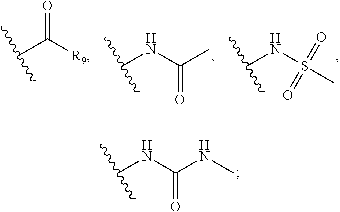

wherein R₂₂ is selected from cycloalkyl, heterocycloalkyl, aryl, heteroaryl,

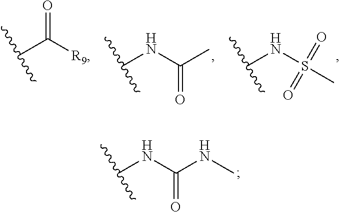

wherein R₂₃ is selected from cycloalkyl, heterocycloalkyl, aryl, heteroaryl, or
(b)₂-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected from alkyl, cycloalkyl, OH, oxo, CF₃, and halo, alkoxy, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O, and X₂ may be further optionally substituted with 1 or 2 substituents R₂₉ and R₃₀, wherein R₂₉ and R₃₀ are independently selected from alkyl, cycloalkyl, alkoxy, OH, oxo, CF₃, and halo; or

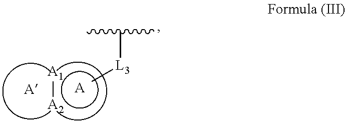

wherein:
L₃ is a single bond;
A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, O(cyclopropyl), CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R, is selected from alkyl, alkoxy, and —(CH₂)₀₋₃-R₈,

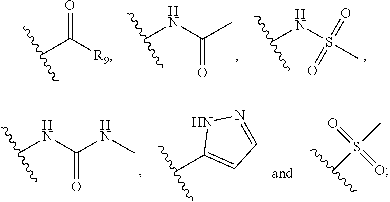

wherein R₈ is selected from cycloalkyl, heterocycloalkyl, wherein R₉ is selected from H, alkyl, OH, N(R₃₁)(R₃₂), and alkoxy;
wherein R₃₁ is H or small alkyl;
wherein R₃₂ is selected from H, small alkyl, and —(CH₂)₀₋₃heteroaryl;
wherein A′ may be optionally substituted with 1, 2, or 3 further substituents R₁₀, R₁₁, and R₁₇, wherein R₁₀, R₁₁, and R₁₇, are independently selected from alkyl, cycloalkyl, alkoxy, OH, oxo, CF₃, and halo, or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O, or wherein A′ may be optionally substituted with 1 or 2 further substituents R₄₃ and R₄₄ independently selected from alkyl, cycloalkyl, alkoxy, OH, oxo, CF₃, and halo, and two different carbon ring members within A′ are connected by a bridging —CH₂-group; and
R₃ can be substituted with one or two substituents independently selected from small alkyl^{b}, small alkoxy,

OH, NH₂, CF₃, halo, —C(═O)—NH₂, and CN.
B₁ can be a carbon centre and B₂ can be a carbon centre, wherein B₃ is a single bond between B₂ and the nitrogen atom to which R₁ is attached and B₁ is connected to B₂ via a single bond, or B₁ can be a nitrogen centre and B₂ can be a carbon centre wherein B₁ is connected to B₂ via a double bond; B₄ can be N(R₂), B₅ can be N, and B₆ can be C(R₃), wherein B₄ is connected to B₅ via a single bond and B₅ is connected to B₆ via a double bond, or B₄ can be C(R₂), B₅ can be N, and B₆ can be N(R₃), wherein B₄ is connected to B₅ via a double bond and B₅ is connected to B₆ via a single bond;
R₁ can be:
(i) Formula (IIb),

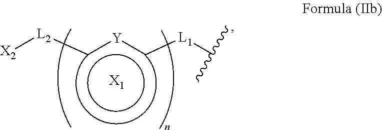

wherein:
n is 1;
L₁ is a linker selected from a single bond and —(CH₂)₁₋₃—;
Y is selected from C, N, O and S;
X₁ is phenyl or a 5- or 6-membered carbon-containing aromatic ring containing one, two or three ring members that are selected from N, S, and O;
wherein X₁ may be optionally substituted with 1 or 2 substituents independently selected from alkyl, cycloalkyl, alkoxy, OH, O(cyclopropyl), CF₃, and halo;
L₂ is a linker selected from a single bond, —(CH₂)₁₋₃—, and —C(═O)—; and
X₂ is either:
(a) —NR₂OR₂₁, wherein R₂₀ is selected from H, alkyl, and —(CH₂)₀₋₃-R_{22;}
wherein R₂₁ is selected from H, alkyl, and —(CH₂)₀₋₃-R₂₃;

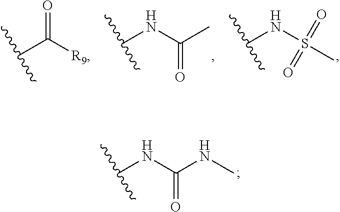

wherein R₂₂ is selected from cycloalkyl, heterocycloalkyl, aryl, heteroaryl,

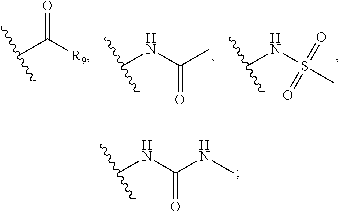

wherein R₂₃ is selected from cycloalkyl, heterocycloalkyl, aryl, heteroaryl, or
(b)₂-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected from alkyl, cycloalkyl, alkoxy, OH, Oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O, and X₂ may be further optionally substituted with 1 or 2 substituents R₂₉ and R₃₀, wherein R₂₉ and R₃₀ are independently selected from alkyl, cycloalkyl, alkoxy, OH, oxo, CF₃, and halo; or
(ii) Formula (III) comprising an aromatic ring A fused to a non-aromatic ring A′,

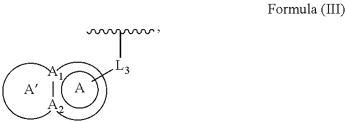

wherein:
L₃ is a single bond;
A₁ and A₂ are both carbon;
A is phenyl or a 5- or 6-membered carbon-containing aromatic ring containing one, two or three
wherein A may be optionally substituted with 1 or 2 substituents independently selected from alkyl, cycloalkyl, alkoxy, OH, O(cyclopropyl), CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R₇ is selected from alkyl, alkoxy, and —(CH₂)₀₋₃-R₈,

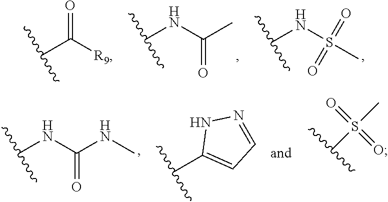

wherein R₈ is selected from cycloalkyl, heterocycloalkyl, wherein R₉ is selected from H, alkyl, OH, N(R₃₁)(R₃₂), and alkoxy;
wherein R₃₁ is H or small alkyl;
wherein R₃₂ is selected from H, small alkyl, and —(CH₂)₀₋₃heteroaryl;
wherein A′ may be optionally substituted with 1, 2, or 3 further substituents R₁₀, R₁₁, and R₁₇, wherein R₁₀, R₁₁, and R₁₇, are independently selected from alkyl, cycloalkyl, alkoxy, OH, oxo, CF₃, and halo, or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O, or wherein A′ may be optionally substituted with 1 or 2 further substituents R₄₃ and R₄₄ independently selected from alkyl, cycloalkyl, alkoxy, OH, oxo, CF₃, and halo, and two different carbon ring members within A′ are connected by a bridging —CH₂-group; and
R₃ can be substituted with one or two substituents independently selected from small alkyl^{b}, small alkoxy, OH, NH₂, CF₃, halo, —C(═O)—NH₂, and CN.
B₁ can be a carbon centre and B₂ can be a carbon centre, wherein B₃ is a single bond between B₂ and the nitrogen atom to which R₁ is attached and B₁ is connected to B₂ via a single bond, or B₁ can be a nitrogen centre and B₂ can be a carbon centre wherein B₁ is connected to B₂ via a double bond; and R₂ can be selected from —C(═O)—N(R₄)(R₅), —C(═O)—O—R₆,—CH₂—NH—C(═O) CH₃,—CH₂—NH—S(═O)₂R₃₅,

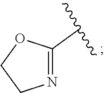 [SMILES: *C1=NCCO1]

—CH₂—O—R₁₆,—CH₂—OH, —CN, and
(i) Formula (IIb),

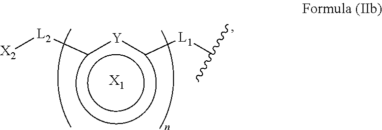

wherein:
n is 1;
L₁ is a linker selected from a single bond and —(CH₂)₁₋₃—; Y is selected from C, N, O and S;
X₁ is phenyl or a 5- or 6-membered carbon-containing aromatic ring containing one, two or three ring members that are selected from N, S, and O;
wherein X₁ may be optionally substituted with 1 or 2 substituents independently selected from alkyl, cycloalkyl, alkoxy, OH, O(cyclopropyl), CF₃, and halo; L₂ is a linker selected from a single bond, —(CH₂)₁₋₃—, and —C(═O)—; and X₂ is either:
(a) —NR₂OR₂₁, wherein R₂₀ is selected from H, alkyl, and —(CH₂)₀₋₃-R_{22;}
wherein R₂₁ is selected from H, alkyl, and —(CH₂)₀₋₃-R₂₃; wherein R₂₂ is selected from cycloalkyl, heterocycloalkyl, aryl, heteroaryl,

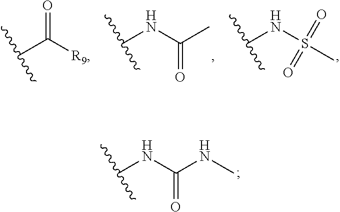

wherein R₂₃ is selected from cycloalkyl, heterocycloalkyl, aryl, heteroaryl,

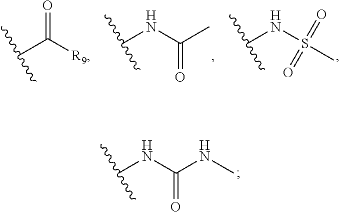

or
(b)₂-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O, and X₂ may be further optionally substituted with 1 or 2 substituents R₂₉ and R₃₀, wherein R₂₉ and R₃₀ are independently selected from alkyl, cycloalkyl, alkoxy, OH, oxo, CF₃, and halo; or
(ii) Formula (III) comprising an aromatic ring A fused to a non-aromatic ring A′,

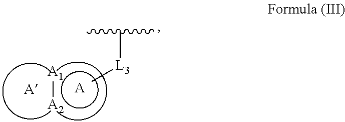

wherein:
L₃ is a single bond;
A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, O(cyclopropyl), CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R₇ is selected from alkyl, alkoxy, and —(CH₂)₀₋₃-R₈,

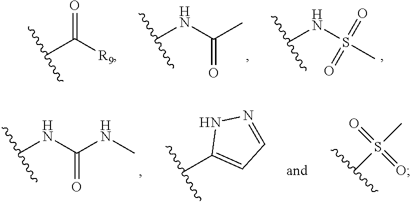

wherein R& is selected from cycloalkyl, heterocycloalkyl, wherein R₉ is selected from H, alkyl, OH, N(R₃₁)(R₃₂), and alkoxy;
wherein R₃₁ is H or small alkyl;
wherein R₃₂ is selected from H, small alkyl, and —(CH₂)₀₋₃heteroaryl;
wherein A′ may be optionally substituted with 1, 2, or 3 further substituents R₁₀, R₁₁, and R₁₇, wherein R₁₀, R₁₁, and R₁₇, are independently selected from alkyl, cycloalkyl, alkoxy, OH, oxo, CF₃, and halo, or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O, or wherein A′ may be optionally substituted with 1 or 2 further substituents R₄₃ and R₄₄ independently selected from alkyl, cycloalkyl, alkoxy, OH, oxo, CF₃, and halo, and two different carbon ring members within A′ are connected by a bridging —CH₂-group; and
R₃ can be substituted with one or two substituents independently selected from small alkyl^{b}, small alkoxy, OH, NH₂, CF₃, halo, —C(═O)—NH₂, and CN.
B₄ can be N(R₂), Bs can be N, and B₆ can be C(R₃), wherein B₄ is connected to B₅ via a single bond and B₅ is connected to B₆ via a double bond, or B₄ can be C(R₂), Bs can be N, and B₆ can be N(R₃), wherein B₄ is connected to B₅ via a double bond and B₅ is connected to B₆ via a single bond; R₂ can be selected from-C(═O)—N(R₄)(R₅), —C(═O)—O—R₆,—CH₂—NH—C(═O) CH₃,—CH₂—NH—S(═O)₂R₃₅,—CH₂—O—R₁₆,—CH₂—OH, —CN, and

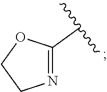 [SMILES: *C1=NCCO1]

R₁ can be:
(i) Formula (IIb),

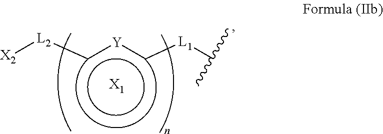

wherein:
n is 1;
L₁ is a linker selected from a single bond and —(CH₂)₁₋₃—; Y is selected from C, N, O and S; X₁ is phenyl or a 5- or 6-membered carbon-containing aromatic ring containing one, two or three
wherein X₁ may be optionally substituted with 1 or 2 substituents independently selected from alkyl, cycloalkyl, alkoxy, OH, O(cyclopropyl), CF₃, and halo; L₂ is a linker selected from a single bond, —(CH₂)₁₋₃—, and —C(═O)—; and
X₂ is either:
(a) —NR₂OR₂₁, wherein R₂₀ is selected from H, alkyl, and —(CH₂)₀₋₃-R_{22;}
wherein R₂₁ is selected from H, alkyl, and —(CH₂)₀₋₃-R₂₃;

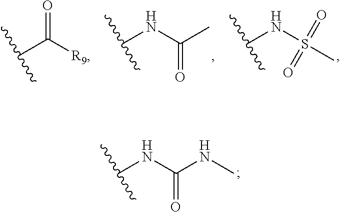

wherein R₂₂ is selected from cycloalkyl, heterocycloalkyl, aryl, heteroaryl,

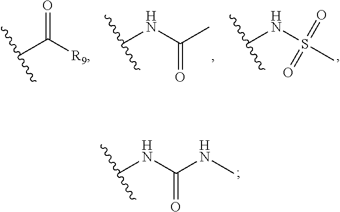

wherein R₂₃ is selected from cycloalkyl, heterocycloalkyl, aryl, heteroaryl, or
(b)₂-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected from alkyl, alkoxy, cycloalkyl, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O, and X₂ may be further optionally substituted with 1 or 2 substituents R₂₉ and R₃₀, wherein R₂₉ and R₃₀ are independently selected from alkyl, cycloalkyl, alkoxy, OH, oxo, CF₃, and halo; or

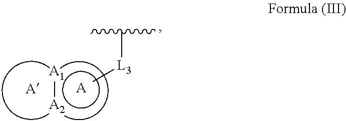

wherein:
L₃ is a single bond;
A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, O(cyclopropyl), CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R₇ is selected from alkyl, alkoxy, and —(CH₂)₀₋₃-R₈,

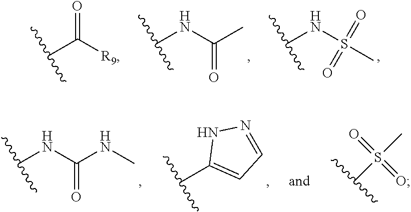

wherein R₈ is selected from cycloalkyl, heterocycloalkyl, wherein R₉ is selected from H, alkyl, OH, N(R₃₁)(R₃₂), and alkoxy;
wherein R₃₁ is H or small alkyl;
wherein R₃₂ is selected from H, small alkyl, and —(CH₂)₀₋₃heteroaryl;
wherein A′ may be optionally substituted with 1, 2, or 3 further substituents R₁₀, R₁₁, and R₁₇, wherein R₁₀, R₁₁, and R₁₇, are independently selected from alkyl, cycloalkyl, alkoxy, OH, oxo, CF₃, and halo, or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O, or wherein A′ may be optionally substituted with 1 or 2 further substituents R₄₃ and R₄₄ independently selected from alkyl, cycloalkyl, alkoxy, OH, oxo, CF₃, and halo, and two different carbon ring members within A′ are connected by a bridging —CH₂-group; and R₃ can be substituted with one or two substituents independently selected from small alkyl^{b}, small alkoxy, OH, NH₂, CF₃, halo, —C(═O)—NH₂, and CN.
B₁ can be a carbon centre and B₂ can be a carbon centre, wherein B₃ is a single bond between B₂ and the nitrogen atom to which R₁ is attached and B₁ is connected to B₂ via a single bond, or B₁ can be a nitrogen centre and B₂ can be a carbon centre wherein B₁ is connected to B₂ via a double bond; B₄ can be N(R₂), B₅ can be N, and B₆ can be C(R₃), wherein B₄ is connected to B₅ via a single bond and B₅ is connected to B₆ via a double bond, or B₄ can be C(R₂), B₅ can be N, and B₆ can be N(R₃), wherein B₄ is connected to B₅ via a double bond and B₅ is connected to B₆ via a single bond; R₂ can be selected from —C(═O)—N(R₄)(R₅),

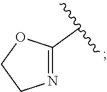 [SMILES: *C1=NCCO1]

—C(═O)—O—R₆,—CH₂—NH—S(═O)₂R₃₅,—CH₂—NH—C(═O) CH₃,—CH₂—O—R₁₆,—CH₂—OH, —CN, and
R₁ can be:
(i) Formula (IIb),

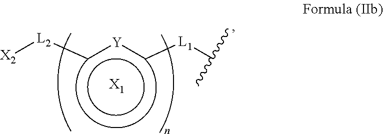

wherein:
n is 1;
L₁ is a linker selected from a single bond and —(CH₂)₁₋₃—;
Y is selected from C, N, O and S;
X₁ is phenyl or a 5- or 6-membered carbon-containing aromatic ring containing one, two or three ring members that are selected from N, S, and O;
wherein X₁ may be optionally substituted with 1 or 2 substituents independently selected from alkyl, cycloalkyl, alkoxy, OH, O(cyclopropyl), CF₃, and halo;
L₂ is a linker selected from a single bond, —(CH₂)₁₋₃—, and —C(═O)—; and
X₂ is either:
(a) —NR₂OR₂₁, wherein R₂₀ is selected from H, alkyl, and —(CH₂)₀₋₃-R_{22;}
wherein R₂₁ is selected from H, alkyl, and —(CH₂)₀₋₃-R₂₃; wherein R₂₂ is selected from cycloalkyl, heterocycloalkyl, aryl, heteroaryl,

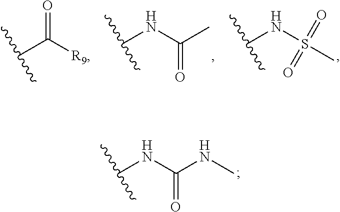

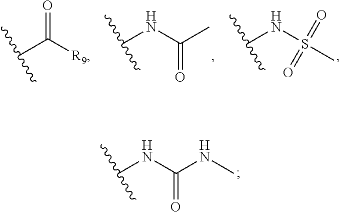

wherein R₂₃ is selected from cycloalkyl, heterocycloalkyl, aryl, heteroaryl, or
(b)₂-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O, and X₂ may be further optionally substituted with 1 or 2 substituents R₂₉ and R₃₀, wherein R₂₉ and R₃₀ are independently selected from alkyl, cycloalkyl, alkoxy, OH, oxo, CF₃, and halo; or
(ii) Formula (III) comprising an aromatic ring A fused to a non-aromatic ring A′,

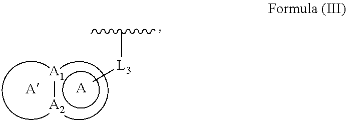

wherein:
L₃ is a single bond;
A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, O(cyclopropyl), CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R₇ is selected from alkyl, alkoxy, and

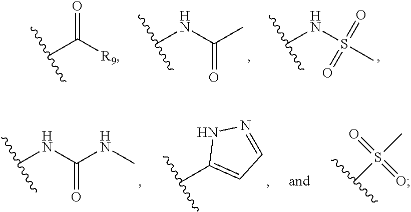

wherein R₈ is selected from cycloalkyl, heterocycloalkyl, wherein R₉ is selected from H, alkyl, OH, N(R₃₁)(R₃₂), and alkoxy;
wherein R₃₁ is H or small alkyl;
wherein R₃₂ is selected from H, small alkyl, and —(CH₂)₀₋₃heteroaryl;
wherein A′ may be optionally substituted with 1, 2, or 3 further substituents Rio, R₁₁, and R₁₇, wherein R₁₀, R₁₁, and R₁₇, are independently selected from alkyl, cycloalkyl, alkoxy, OH, oxo, CF₃, and halo, or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O, or wherein A′ may be optionally substituted with 1 or 2 further substituents R₄₃ and R₄₄ independently selected from alkyl, cycloalkyl, alkoxy, OH, oxo, CF₃, and halo, and two different carbon ring members within A′ are connected by a bridging —CH₂-group; and
R₃ can be substituted with one or two substituents independently selected from small alkyl^{b}, small alkoxy,

OH, NH₂, CF₃, halo, —C(═O)—NH₂, and CN.
R₂ can be selected from —C(═O)—N(R₄)(R₅), —C(═O)—O—R₆,—CH₂—NH—S(═O)₂R₃₅,—CH₂—NH—C(═O) CH₃,—CH₂—O—R₁₆,

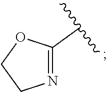 [SMILES: *C1=NCCO1]

—CH₂—OH, —CN, and
R₁ can be:
(i) Formula (IIb),

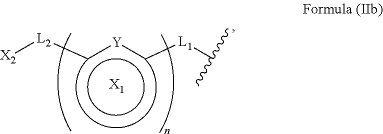

wherein:
n is 1;
L₁ is a linker selected from a single bond and —(CH₂)₁₋₃—;
Y is selected from C, N, O and S;
X₁ is phenyl or a 5- or 6-membered carbon-containing aromatic ring containing one, two or three ring members that are selected from N, S, and O;
wherein X₁ may be optionally substituted with 1 or 2 substituents independently selected from alkyl, cycloalkyl, alkoxy, OH, O(cyclopropyl), CF₃, and halo;
L₂ is a linker selected from a single bond, —(CH₂)₁₋₃—, and —C(═O)—; and
X₂ is either:
(a) —NR₂OR₂₁, wherein R₂₀ is selected from H, alkyl, and —(CH₂)₀₋₃-R_{22;}
wherein R₂₁ is selected from H, alkyl, and —(CH₂)₀₋₃-R₂₃; <CWU-Call number= “666” />wherein R₂₂ is selected from cycloalkyl, heterocycloalkyl, aryl, heteroaryl,

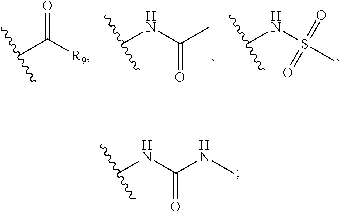

wherein R₂₃ is selected from cycloalkyl, heterocycloalkyl, aryl, heteroaryl,

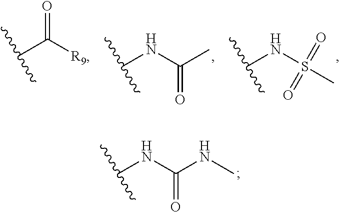

or
(b)₂-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O, and X₂ may be further optionally substituted with 1 or 2 substituents R₂₉ and R₃₀, wherein R₂₉ and R₃₀ are independently selected from alkyl, cycloalkyl, alkoxy, OH, oxo, CF₃, and halo; or

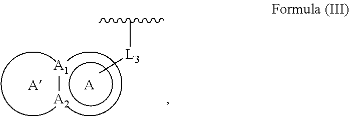

wherein:
L₃ is a single bond;
A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, O(cyclopropyl), CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R₇ is selected from alkyl, alkoxy, and —(CH₂)₀₋₃-R₈,

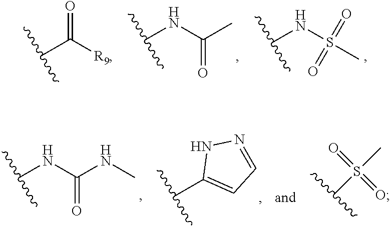

wherein R₈ is selected from cycloalkyl, heterocycloalkyl, wherein R₉ is selected from H, alkyl, OH, N(R₃₁)(R₃₂), and alkoxy;
wherein R₃₁ is H or small alkyl;
wherein R₃₂ is selected from H, small alkyl, and —(CH₂)₀₋₃heteroaryl;
wherein A′ may be optionally substituted with 1, 2, or 3 further substituents R₁₀, R₁₁, and R₁₇, wherein R₁₀, R₁₁, and R₁₇, are independently selected from alkyl, cycloalkyl, alkoxy, OH, oxo, CF₃, and halo, or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O, or wherein A′ may be optionally substituted with 1 or 2 further substituents R₄₃ and R₄₄ independently selected from alkyl, cycloalkyl, alkoxy, OH, oxo, CF₃, and halo, and two different carbon ring members within A′ are connected by a bridging —CH₂-group; and
R₃ can be substituted with one or two substituents independently selected from small alkyl^{b}, small alkoxy,

OH, NH₂, CF₃, halo, —C(═O)—NH₂, and CN.
B₁ can be a carbon centre and B₂ can be a carbon centre, wherein B₃ is a single bond between B₂ and the nitrogen atom to which R₁ is attached and B₁ is connected to B₂ via a single bond, or B₁ can be a nitrogen centre and B₂ can be a carbon centre wherein B₁ is connected to B₂ via a double bond; R₂ can be selected from —C(═O)—N(R₄)(R₅), —C(═O)—O—R₆,—CH₂—NH—C(═O) CH₃,—CH₂—NH—S(═O)₂R₃₅,—CH₂—O—R₁₆,—CH₂—OH, —CN, and

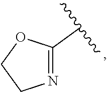

wherein:
R₄ is H or small alkyl, and
R₅ is selected from alkyl, OH, —S(═O)₂-(small alkyl), —(CH₂)₁₋₃—(C═O)—OH, —(CH₂)₀₋₃-cycloalkyl^{b}, —(CH₂)₀₋₃-heterocycloalkyl, —(CH(alkyl)-heterocycloalkyl, —(CH₂)₀₋₃-polycycloalkyl, —(CH₂)₀₋₃-aryl, —(C(H)(phenyl)-alkyl, —(CH₂)₀₋₃heteroaryl, —(C(H)(heteroaryl))-alkyl, —(C(H)(alkyl))—(C═O)—OH, and —(C(H)(heteroaryl))—(C═O)—OR_{28;}
or R₄ and R₅ together with the nitrogen atom to which they are attached can form a carbon-containing 4-, 5-, or 6-membered saturated heterocyclic ring, optionally containing an additional heteroatom selected from N, and O, which may be optionally mono- or di-substituted with substituents selected from oxo, alkyl, alkoxy, OH, halo and CF_{3;}
R₆ is H, small alkyl, and —(CH₂)-phenyl;
R₁₆ is selected from H, small alkyl, —(CH₂)—(C═O)—OH, heterocycloalkyl, and —(CH₂)-heteroaryl; and R₃₅ is selected from H, small alkyl, and cyclopropyl;
R₁ can be:
(i) Formula (IIb),

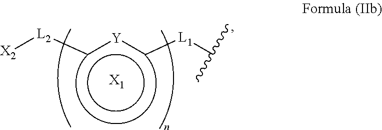

wherein:
n is 1;
L₁ is a linker selected from a single bond and —(CH₂)₁₋₃—;
Y is selected from C, N, O and S;
X₁ is phenyl or a 5- or 6-membered carbon-containing aromatic ring containing one, two or three ring members that are selected from N, S, and O;
wherein X₁ may be optionally substituted with 1 or 2 substituents independently selected from alkyl, cycloalkyl, alkoxy, OH, O(cyclopropyl), CF₃, and halo; L₂ is a linker selected from a single bond, —(CH₂)₁₋₃—, and —C(═O)—; and
X₂ is either:
(a) —NR₂OR₂₁, wherein R₂₀ is selected from H, alkyl, and —(CH₂)₀₋₃-R_{22;}
wherein R₂₁ is selected from H, alkyl, and —(CH₂)₀₋₃-R₂₃; wherein R₂₂ is selected from cycloalkyl, heterocycloalkyl, aryl, heteroaryl,

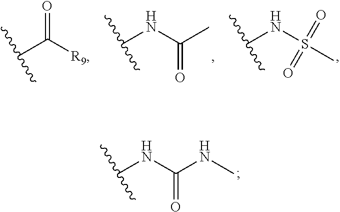

wherein R₂₃ is selected from cycloalkyl, heterocycloalkyl, aryl, heteroaryl,

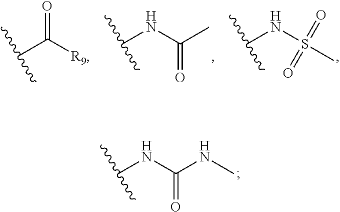

or
(b)₂-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O, and X₂ may be further optionally substituted with 1 or 2 substituents R₂₉ and R₃₀, wherein R₂₉ and R₃₀ are independently selected from alkyl, cycloalkyl, alkoxy, OH, oxo, CF₃, and halo; or

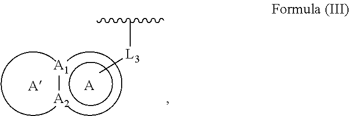

wherein:
L₃ is a single bond;
A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, O(cyclopropyl), CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R, is selected from alkyl, alkoxy, and —(CH₂)₀₋₃-R₈,

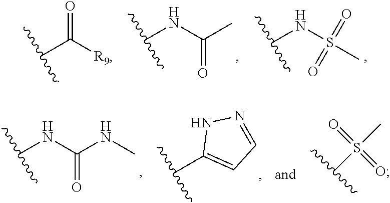

wherein R₈ is selected from cycloalkyl, heterocycloalkyl, wherein R₉ is selected from H, alkyl, OH, N(R₃₁)(R₃₂), and alkoxy;
wherein R₃₁ is H or small alkyl;
wherein R₃₂ is selected from H, small alkyl, and —(CH₂)₀₋₃heteroaryl;
wherein A′ may be optionally substituted with 1, 2, or 3 further substituents R₁₀, R₁₁, and R₁₇, wherein R₁₀, R₁₁, and R₁₇, are independently selected from alkyl, cycloalkyl, alkoxy, OH, oxo, CF₃, and halo, or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O, or wherein A′ may be optionally substituted with 1 or 2 further substituents R₄₃ and R₄₄ independently selected from alkyl, cycloalkyl, alkoxy, OH, oxo, CF₃, and halo, and two different carbon ring members within A′ are connected by a bridging —CH₂-group; and
R₃ can be substituted with one or two substituents independently selected from small alkyl^{b}, small alkoxy,

OH, NH₂, CF₃, halo, —C(═O)—NH₂, and CN.
B₄ can be N(R₂), B₅ can be N, and B₆ can be C(R₃), wherein B₄ is connected to B₅ via a single bond and B₅ is connected to B₆ via a double bond, or B₄ can be C(R₂), Bs can be N, and B₆ can be N(R₃), wherein B₄ is connected to Bs via a double bond and B₅ is connected to B₆ via a single bond; R₂ can be selected from-C(═O)—N(R₄)(R₅), —C(═O)—O—R₆,—CH₂—NH—C(═O) CH₃,—CH₂—NH—S(═O)₂R₃₅,—CH₂—O—R₁₆,—CH₂—OH, —CN, and

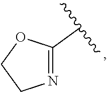

wherein:
R₄ is H or small alkyl, and
R₅ is selected from alkyl, OH, —S(═O)₂-(small alkyl), —(CH₂)₁₋₃—(C═O)—OH, —(CH₂)₀₋₃-cycloalkyl^{b}, —(CH₂)₀₋₃-heterocycloalkyl, —(CH(alkyl)-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, —(C(H)(heteroaryl))-alkyl, —(C(H)(alkyl))—(C═O)—OH, and —(C(H)(heteroaryl))—(C═O)—OR_{28;}
or R₄ and R₅ together with the nitrogen atom to which they are attached can form a carbon-containing 4-, 5-, or 6-membered saturated heterocyclic ring, optionally containing an additional heteroatom selected from N, and O, which may be optionally mono- or di-substituted with substituents selected from oxo, alkyl, alkoxy, OH, halo and CF_{3;}
R₆ is H, small alkyl, and —(CH₂)-phenyl;
R₁₆ is selected from H, small alkyl, —(CH₂)—(C═O)—OH, heterocycloalkyl, and —(CH₂)-heteroaryl; and R₃₅ is selected from H, small alkyl, and cyclopropyl;
R₁ can be:
(i) Formula (IIb),

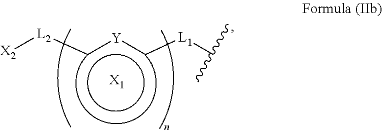

wherein:
n is 1;
L₁ is a linker selected from a single bond and —(CH₂)₁₋₃—; Y is selected from C, N, O and S;
X₁ is phenyl or a 5- or 6-membered carbon-containing aromatic ring containing one, two or three ring members that are selected from N, S, and O;
wherein X₁ may be optionally substituted with 1 or 2 substituents independently selected from alkyl, cycloalkyl, alkoxy, OH, O(cyclopropyl), CF₃, and halo; L₂ is a linker selected from a single bond, —(CH₂)₁₋₃—, and —C(═O)—; and X₂ is either:
(a) —NR₂OR₂₁, wherein R₂₀ is selected from H, alkyl, and —(CH₂)₀₋₃-R_{2;}
wherein R₂₁ is selected from H, alkyl, and —(CH₂)₀₋₃-R₂₃;

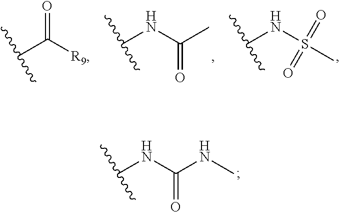

wherein R₂₂ is selected from cycloalkyl, heterocycloalkyl, aryl, heteroaryl,

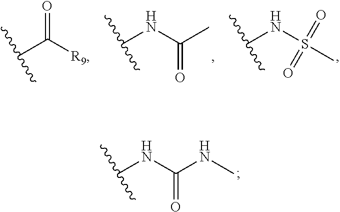

wherein R₂₃ is selected from cycloalkyl, heterocycloalkyl, aryl, heteroaryl, or
(b)₂-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected from alkyl, cycloalkyl, OH, alkoxy, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O, and X₂ may be further optionally substituted with 1 or 2 substituents R₂₉ and R₃₀, wherein R₂₉ and R₃₀ are independently selected from alkyl, cycloalkyl, alkoxy, OH, oxo, CF₃, and halo; or

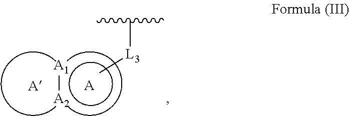

wherein:
L₃ is a single bond;
A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, O(cyclopropyl), CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R, is selected from alkyl, alkoxy, and —(CH₂)₀₋₃-R₈,

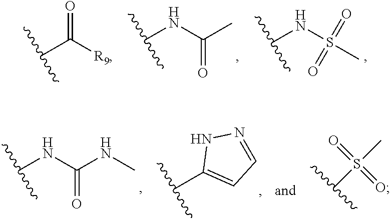

wherein R₈ is selected from cycloalkyl, heterocycloalkyl, wherein R₉ is selected from H, alkyl, OH, N(R₃₁)(R₃₂), and alkoxy;
wherein R₃₁ is H or small alkyl;
wherein R₃₂ is selected from H, small alkyl, and —(CH₂)₀₋₃heteroaryl;
wherein A′ may be optionally substituted with 1, 2, or 3 further substituents R₁₀, R₁₁, and R₁₇, wherein R₁₀, R₁₁, and R₁₇, are independently selected from alkyl, cycloalkyl, alkoxy, OH, oxo, CF₃, and halo, or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O, or wherein A′ may be optionally substituted with 1 or 2 further substituents R₄₃ and R₄₄ independently selected from alkyl, cycloalkyl, alkoxy, OH, oxo, CF₃, and halo, and two different carbon ring members within A′ are connected by a bridging —CH₂-group; and
R₃ can be substituted with one or two substituents independently selected from small alkyl^{b}, small alkoxy,

OH, NH₂, CF₃, halo, —C(═O)—NH₂, and CN.
B₁ can be a carbon centre and B₂ can be a carbon centre, wherein B₃ is a single bond between B₂ and the nitrogen atom to which R₁ is attached and B₁ is connected to B₂ via a single bond, or B₁ can be a nitrogen centre and B₂ can be a carbon centre wherein B₁ is connected to B₂ via a double bond; B₄ can be N(R₂), B₅ can be N, and B₆ can be C(R₃), wherein B₄ is connected to B₅ via a single bond and B₅ is connected to B₆ via a double bond, or B₄ can be C(R₂), B₅ can be N, and B₆ can be N(R₃), wherein B₄ is connected to B₅ via a double bond and B₅ is connected to B₆ via a single bond; R₂ can be selected from —C(═O)—N(R₄)(R₅),

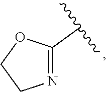 [SMILES: *C1=NCCO1]

—C(═O)—O—R₆,—CH₂—NH—S(═O)₂R₃₅,—CH₂—NH—C(═O) CH₃,—CH₂—O—R₁₆,—CH₂—OH, —CN, and wherein:
R₄ is H or small alkyl, and
R₅ is selected from alkyl, OH, —S(═O)₂-(small alkyl), —(CH₂)₁₋₃—(C═O)—OH, —(CH₂)₀₋₃-cycloalkyl^{b}, —(CH₂)₀₋₃-heterocycloalkyl, —(CH(alkyl)-heterocycloalkyl, —(CH₂)₀₋₃-polycycloalkyl, —(CH₂)₀₋₃-aryl, —(C(H)(phenyl))-alkyl,-—(C(H)(heteroaryl))-alkyl, —(C(H)(alkyl))—(C═O)—OH, and —(C(H)(heteroaryl))—(C═O)—OR_{28;}
or R₄ and R₅ together with the nitrogen atom to which they are attached can form a carbon-containing 4-, 5-, or 6-membered saturated heterocyclic ring, optionally containing an additional heteroatom selected from N, and O, which may be optionally mono- or di-substituted with substituents selected from oxo, alkyl, alkoxy, OH, halo and CF₃; R₆ is H, small alkyl, and —(CH₂)-phenyl;
R₁₆ is selected from H, small alkyl, —(CH₂)—(C═O)—OH, heterocycloalkyl, and —(CH₂)-heteroaryl; and
R₃₅ is selected from H, small alkyl, and cyclopropyl;
R₁ can be:
(i) Formula (IIb),

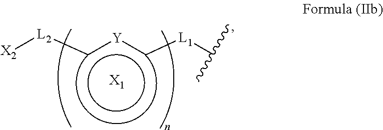

wherein:
n is 1;
L₁ is a linker selected from a single bond and —(CH₂)₁₋₃—;
Y is selected from C, N, O and S;
X₁ is phenyl or a 5- or 6-membered carbon-containing aromatic ring containing one, two or three ring members that are selected from N, S, and O;
wherein X₁ may be optionally substituted with 1 or 2 substituents independently selected from alkyl, cycloalkyl, alkoxy, OH, O(cyclopropyl), CF₃, and halo;
L₂ is a linker selected from a single bond, —(CH₂)₁₋₃—, and —C(═O)—; and
X₂ is either:
(a) —NR₂OR₂₁, wherein R₂₀ is selected from H, alkyl, and —(CH₂)₀₋₃-R_{22;}
wherein R₂₁ is selected from H, alkyl, and —(CH₂)₀₋₃-R₂₃;

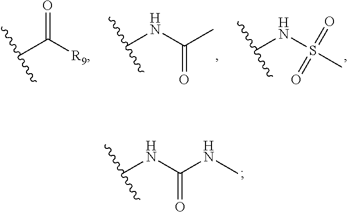

wherein R₂₂ is selected from cycloalkyl, heterocycloalkyl, aryl, heteroaryl,

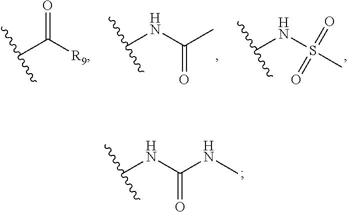

wherein R₂₃ is selected from cycloalkyl, heterocycloalkyl, aryl, heteroaryl, or
(b)₂-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected from alkyl, cycloalkyl, OH, oxo, CF₃, and halo, alkoxy, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O, and X₂ may be further optionally substituted with 1 or 2 substituents R₂₉ and R₃₀, wherein R₂₉ and R₃₀ are independently selected from alkyl, cycloalkyl, alkoxy, OH, oxo, CF₃, and halo; or

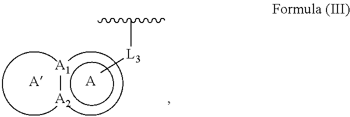

wherein:
L₃ is a single bond;
A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, O(cyclopropyl), CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R₇ is selected from alkyl, alkoxy, and —(CH₂)₀₋₃-R₈,

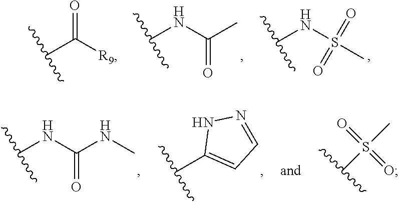

wherein R₈ is selected from cycloalkyl, heterocycloalkyl, wherein R₉ is selected from H, alkyl, OH, N(R₃₁)(R₃₂), and alkoxy;
wherein R₃₁ is H or small alkyl;
wherein R₃₂ is selected from H, small alkyl, and —(CH₂)₀₋₃heteroaryl;
wherein A′ may be optionally substituted with 1, 2, or 3 further substituents R₁₀, R₁₁, and R₁₇, wherein R₁₀, R₁₁, and R₁₇, are independently selected from alkyl, cycloalkyl, alkoxy, OH, oxo, CF₃, and halo, or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O, or wherein A′ may be optionally substituted with 1 or 2 further substituents R₄₃ and R₄₄ independently selected from alkyl, cycloalkyl, alkoxy, OH, oxo, CF₃, and halo, and two different carbon ring members within A′ are connected by a bridging —CH₂-group; and
R₃ can be substituted with one or two substituents independently selected from small alkyl^{b}, small alkoxy,

OH, NH₂, CF₃, halo, —C(═O)—NH₂, and CN.
R₂ can be selected from —C(═O)—N(R₄)(R₅), —C(═O)—O—R₆,—CH₂—NH—S(═O)₂R₃₅,—CH₂—NH—C(═O) CH₃,—CH₂—O—R₁₆,

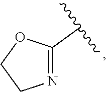 [SMILES: *C1=NCCO1]

—CH₂—OH, —CN, and wherein:
R₄ is H or small alkyl, and
R₅ is selected from alkyl, OH, —S(═O)₂-(small alkyl), —(CH₂)₁₋₃—(C═O)—OH, —(CH₂)₀₋₃-cycloalkyl^{b}, —(CH₂)₀₋₃-heterocycloalkyl, —(CH(alkyl)-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, —(C(H)(heteroaryl))-alkyl, —(C(H)(alkyl))—(C═O)—OH, and —(C(H)(heteroaryl))—(C═O)—OR_{28;}
or R₄ and R₅ together with the nitrogen atom to which they are attached can form a carbon-containing 4-, 5-, or 6-membered saturated heterocyclic ring, optionally containing an additional heteroatom selected from N, and O, which may be optionally mono- or di-substituted with substituents selected from oxo, alkyl, alkoxy, OH, halo and CF₃; R₆ is H, small alkyl, and —(CH₂)-phenyl;
R₁₆ is selected from H, small alkyl, —(CH₂)—(C═O)—OH, heterocycloalkyl, and —(CH₂)-heteroaryl; and
R₃₅ is selected from H, small alkyl, and cyclopropyl;
R₁ can be:
(i) Formula (IIb),

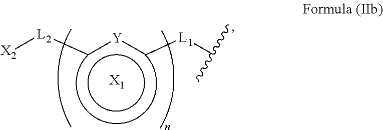

wherein:
n is 1;
L₁ is a linker selected from a single bond and —(CH₂)₁₋₃—;
Y is selected from C, N, O and S;
X₁ is phenyl or a 5- or 6-membered carbon-containing aromatic ring containing one, two or three ring members that are selected from N, S, and O;
wherein X₁ may be optionally substituted with 1 or 2 substituents independently selected from alkyl, cycloalkyl, alkoxy, OH, O(cyclopropyl), CF₃, and halo; L₂ is a linker selected from a single bond, —(CH₂)₁₋₃—, and —C(═O)—; and
X₂ is either:
(a) —NR₂OR₂₁, wherein R₂₀ is selected from H, alkyl, and —(CH₂)₀₋₃-R_{2;}
wherein R₂₁ is selected from H, alkyl, and —(CH₂)₀₋₃-R₂₃;

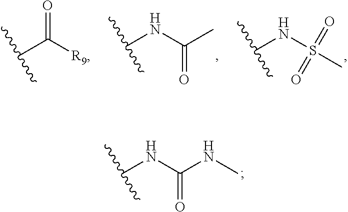

wherein R₂₂ is selected from cycloalkyl, heterocycloalkyl, aryl, heteroaryl,

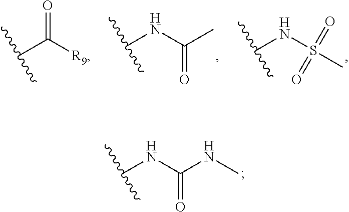

wherein R₂₃ is selected from cycloalkyl, heterocycloalkyl, aryl, heteroaryl, or
(b)₂-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O, and X₂ may be further optionally substituted with 1 or 2 substituents R₂₉ and R₃₀, wherein R₂₉ and R₃₀ are independently selected from alkyl, cycloalkyl, alkoxy, OH, oxo, CF₃, and halo; or

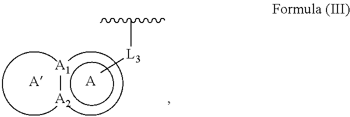

wherein:
L₃ is a single bond;
A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, O(cyclopropyl), CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R₇ is selected from alkyl, alkoxy, and —(CH₂)₀₋₃-R₈,

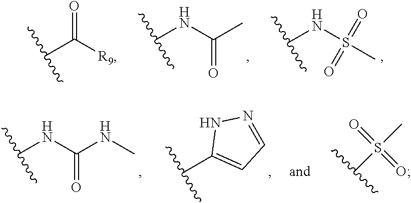

wherein R₈ is selected from cycloalkyl, heterocycloalkyl, wherein R₉ is selected from H, alkyl, OH, N(R₃₁)(R₃₂), and alkoxy;
wherein R₃₁ is H or small alkyl;
wherein R₃₂ is selected from H, small alkyl, and —(CH₂)₀₋₃heteroaryl;
wherein A′ may be optionally substituted with 1, 2, or 3 further substituents R₁₀, R₁₁, and R₁₇, wherein R₁₀, R₁₁, and R₁₇, are independently selected from alkyl, cycloalkyl, alkoxy, OH, oxo, CF₃, and halo, or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O, or wherein A′ may be optionally substituted with 1 or 2 further substituents R₄₃ and R₄₄ independently selected from alkyl, cycloalkyl, alkoxy, OH, oxo, CF₃, and halo, and two different carbon ring members within A′ are connected by a bridging —CH₂-group; and
R₃ can be substituted with one or two substituents independently selected from small alkyl^{b}, small alkoxy, OH, NH₂, CF₃, halo, —C(═O)—NH₂, and CN.
R₁ can be:
(i) Formula (IIb),

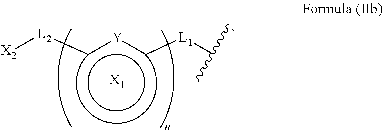

wherein:
n is 1;
L₁ is a linker selected from a single bond and —(CH₂)₁₋₃—;
Y is selected from C, N, O and S;
X₁ is phenyl or a 5- or 6-membered carbon-containing aromatic ring containing one, two or three ring members that are selected from N, S, and O;
wherein X₁ may be optionally substituted with 1 or 2 substituents independently selected from alkyl, cycloalkyl, alkoxy, OH, O(cyclopropyl), CF₃, and halo; L₂ is a linker selected from a single bond, —(CH₂)₁₋₃—, and —C(═O)—; and
X₂ is either:
(a) —NR₂OR₂₁, wherein R₂₀ is selected from H, alkyl, and —(CH₂)₀₋₃-R_{22;}
wherein R₂₁ is selected from H, alkyl, and —(CH₂)₀₋₃-R₂₃;

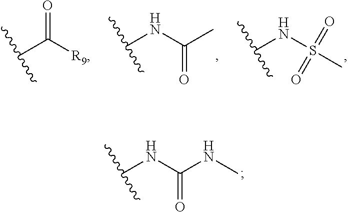

wherein R₂₂ is selected from cycloalkyl, heterocycloalkyl, aryl, heteroaryl,

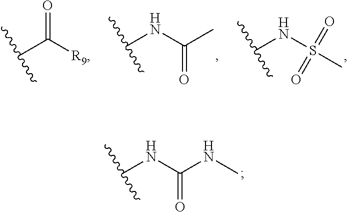

wherein R₂₃ is selected from cycloalkyl, heterocycloalkyl, aryl, heteroaryl, or
(b)₂-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O, and X₂ may be further optionally substituted with 1 or 2 substituents R₂₉ and R₃₀, wherein R₂₉ and R₃₀ are independently selected from alkyl, cycloalkyl, alkoxy, OH, oxo, CF₃, and halo; or
(ii) Formula (III) comprising an aromatic ring A fused to a non-aromatic ring A′,

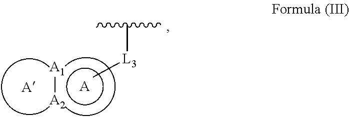

wherein:
L₃ is a single bond;
A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, O(cyclopropyl), CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R₇ is selected from alkyl, alkoxy, and —(CH₂)₀₋₃-R₈,

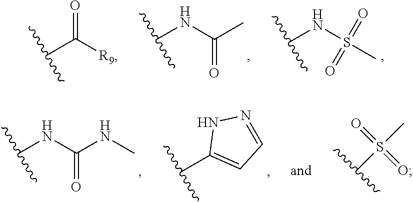

wherein R₈ is selected from cycloalkyl, heterocycloalkyl, wherein R₉ is selected from H, alkyl, OH, N(R₃₁)(R₃₂), and alkoxy;
wherein R₃₁ is H or small alkyl;
wherein R₃₂ is selected from H, small alkyl, and —(CH₂)₀₋₃heteroaryl;
wherein A′ may be optionally substituted with 1, 2, or 3 further substituents R₁, R₁₁, and R₁₇, wherein R₁₀, R₁₁, and R₁₇, are independently selected from alkyl, cycloalkyl, alkoxy, OH, oxo, CF₃, and halo, or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O, or wherein A′ may be optionally substituted with 1 or 2 further substituents R₄₃ and R₄₄ independently selected from alkyl, cycloalkyl, alkoxy, OH, oxo, CF₃, and halo, and two different carbon ring members within A′ are connected by a bridging —CH₂-group; and
R₃ can be substituted with one or two substituents independently selected from small alkyl^{b}, small alkoxy, OH, NH₂, CF₃, halo, —C(═O)—NH₂, and CN.
B₁ can be a carbon centre and B₂ can be a carbon centre, wherein B₃ is a single bond between B₂ and the nitrogen atom to which R₁ is attached and B₁ is connected to B₂ via a single bond, or B₁ can be a nitrogen centre and B₂ can be a carbon centre wherein B₁ is connected to B₂ via a double bond; and R₃ can be

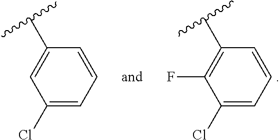 [SMILES: *c1cccc(Cl)c1 ; *c1cccc(Cl)c1F]

selected from B₄ can be N(R₂), B₅ can be N, and B₆ can be C(R₃), wherein B₄ is connected to B₅ via a single bond and B₅ is connected to B₆ via a double bond, or B₄ can be C(R₂), B₅ can be N, and B₆ can be N(R₃), wherein B₄ is connected to B₅ via a double bond and B₅ is connected to B₆ via a single bond; and R₃ can be selected

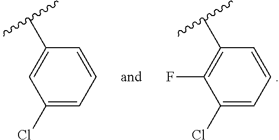 [SMILES: *c1cccc(Cl)c1 ; *c1cccc(Cl)c1F]

from B₁ can be a carbon centre and B₂ can be a carbon centre, wherein B₃ is a single bond between B₂ and the nitrogen atom to which R₁ is attached and B₁ is connected to B₂ via a single bond, or B₁ can be a nitrogen centre and B₂ can be a carbon centre wherein B₁ is connected to B₂ via a double bond; B₄ can be N(R₂), B₅ can be N, and B₆ can be C(R₃), wherein B₄ is connected to B₅ via a single bond and B₅ is connected to B₆ via a double bond, or B₄ can be C(R₂), Bs can be N, and B₆ can be N(R₃), wherein B₄ is connected to Bs via a

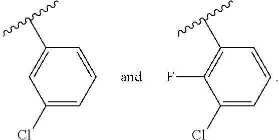 [SMILES: *c1cccc(Cl)c1 ; *c1cccc(Cl)c1F]

double bond and B₅ is connected to B₆ via a single bond; and R₃ can be selected from B₁ can be a carbon centre and B₂ can be a carbon centre, wherein B₃ is a single bond between B₂ and the nitrogen atom to which R₁ is attached and B₁ is connected to B₂ via a single bond, or B₁ can be a nitrogen centre and B₂ can be a carbon centre wherein B₁ is connected to B₂ via a double bond; and R₂ can be selected from —C(═O)—N(R₄)(R₅), —C(═O)—O—R₆,—CH₂—NH—C(═O) CH₃,—CH₂—NH—S(═O)₂R₃₅,

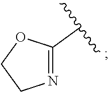 [SMILES: *C1=NCCO1]

—CH₂—O—R₁₆,—CH₂—OH, —CN, and and R₃ can be selected from

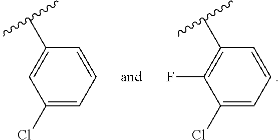 [SMILES: *c1cccc(Cl)c1 ; *c1cccc(Cl)c1F]

B₄ can be N(R₂), B₅ can be N, and B₆ can be C(R₃), wherein B₄ is connected to B₅ via a single bond and B₅ is connected to B₆ via a double bond, or B₄ can be C(R₂), Bs can be N, and B₆ can be N(R₃), wherein B₄ is connected to B₅ via a double bond and B₅ is connected to B₆ via a single bond; R₂ can be selected from-C(═O)—N(R₄)(R₅), —C(═O)—O—R₆,—CH₂—NH—C(═O) CH₃,—CH₂—NH—S(═O)₂R₃₅,—CH₂—O—R₁₆,—CH₂—OH, —CN, and

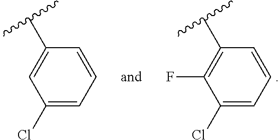 [SMILES: *c1cccc(Cl)c1 ; *c1cccc(Cl)c1F]

and R₃ can be selected from

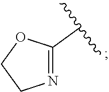 [SMILES: *C1=NCCO1]

B₁ can be a carbon centre and B₂ can be a carbon centre, wherein B₃ is a single bond between B₂ and the nitrogen atom to which R₁ is attached and B₁ is connected to B₂ via a single bond, or B₁ can be a nitrogen centre and B₂ can be a carbon centre wherein B₁ is connected to B₂ via a double bond; B₄ can be N(R₂), B₅ can be N, and B₆ can be C(R₃), wherein B₄ is connected to B₅ via a single bond and B₅ is connected to B₆ via a double bond, or B₄ can be C(R₂), B₅ can be N, and B₆ can be N(R₃), wherein B₄ is connected to B₅ via a double bond and B₅ is connected to B₆ via a single bond; R₂ can be selected from —C(═O)—N(R₄)(R₅),

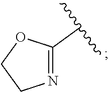 [SMILES: *C1=NCCO1]

—C(═O)—O—R₆,—CH₂—NH—S(═O)₂R₃₅,—CH₂—NH—C(═O) CH₃,—CH₂—O—R₁₆,—CH₂—OH, —CN, and

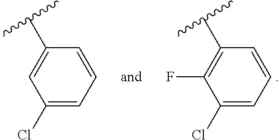 [SMILES: *c1cccc(Cl)c1 ; *c1cccc(Cl)c1F]

and R₃ can be selected from R₂ can be selected from —C(═O)—N(R₄)(R₅), —C(═O)—O—R₆,—CH₂—NH—C(═O) CH₃,—CH₂—NH—S(═O)₂R₃₅,—CH₂—O—R₁₆,

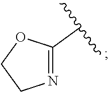 [SMILES: *C1=NCCO1]

—CH₂—OH, —CN, and and R₃ can be selected from

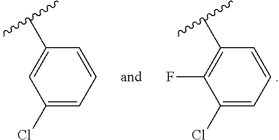 [SMILES: *c1cccc(Cl)c1 ; *c1cccc(Cl)c1F]

B₁ can be a carbon centre and B₂ can be a carbon centre, wherein B₃ is a single bond between B₂ and the nitrogen atom to which R₁ is attached and B₁ is connected to B₂ via a single bond, or B₁ can be a nitrogen centre and B₂ can be a carbon centre wherein B₁ is connected to B₂ via a double bond; and R₂ can be selected from —C(═O)—N(R₄)(R₅), —C(═O)—O—R₆,—CH₂—NH—C(═O) CH₃,—CH₂—NH—S(═O)₂R₃₅,—CH₂—O—R₁₆,—CH₂—OH, —

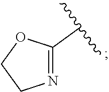 [SMILES: *C1=NCCO1]

CN, and wherein:
R₄ is H or small alkyl, and
R₅ is selected from alkyl, OH, —S(═O)₂-(small alkyl), —(CH₂)₁₋₃—(C═O)—OH, —(CH₂)₀₋₃-cycloalkyl^{b}, —(CH₂)₀₋₃-heterocycloalkyl, —(CH(alkyl))-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, —(C(H)(heteroaryl))-alkyl, —(C(H)(alkyl))—(C═O)—OH, and —(C(H)(heteroaryl))—(C═O)—OR_{28;}
or R₄ and R₅ together with the nitrogen atom to which they are attached can form a carbon-containing 4-, 5-, or 6-membered saturated heterocyclic ring, optionally containing an additional heteroatom selected from N, and O, which may be optionally mono- or di-substituted with substituents selected from oxo, alkyl, alkoxy, OH, halo and CF_{3;}
R₆ is H, small alkyl, and —(CH₂)-phenyl; R₁₆ is selected from H, small alkyl, —(CH₂)—(C═O)—OH, heterocycloalkyl, and —(CH₂)-heteroaryl; and R₃₅ is selected from H, small alkyl, and cyclopropyl; and

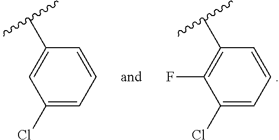 [SMILES: *c1cccc(Cl)c1 ; *c1cccc(Cl)c1F]

R₃ can be selected from B₄ can be N(R₂), B₅ can be N, and B₆ can be C(R₃), wherein B₄ is connected to Bs via a single bond and B₅ is connected to B₆ via a double bond, or B₄ can be C(R₂), B₅ can be N, and B₆ can be N(R₃), wherein B₄ is connected to B₅ via a double bond and B₅ is connected to B₆ via a single bond; R₂ can be selected from-C(═O)—N(R₄)(R₅), —C(═O)—O—R₆,—CH₂—NH—C(═O) CH₃,—CH₂—NH—S(═O)₂R₃₅,—CH₂—O—R₁₆,—CH₂—OH, —CN, and

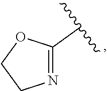

wherein:
R₄ is H or small alkyl, and
R₅ is selected from alkyl, OH, —S(═O)₂-(small alkyl), —(CH₂)₁₋₃—(C═O)—OH, —(CH₂)₀₋₃-cycloalkyl^{b}, —(CH₂)₀₋₃-heterocycloalkyl, —(CH(alkyl))-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, —(C(H)(heteroaryl))-alkyl, —(C(H)(alkyl))—(C═O)—OH, and —(C(H)(heteroaryl))—(C═O)—OR_{28;}
or R₄ and R₅ together with the nitrogen atom to which they are attached can form a carbon-containing 4-, 5-, or 6-membered saturated heterocyclic ring, optionally containing an additional heteroatom selected from N, and O, which may be optionally mono- or di-substituted with substituents selected from oxo, alkyl, alkoxy, OH, halo and CF_{3;}
R₆ is H, small alkyl, and —(CH₂)-phenyl; R₁₆ is selected from H, small alkyl, —(CH₂)—(C═O)—OH, heterocycloalkyl, and —(CH₂)-heteroaryl; and R₃₅ is selected from H, small alkyl, and cyclopropyl; and

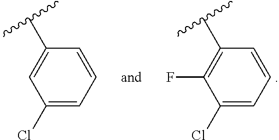 [SMILES: *c1cccc(Cl)c1 ; *c1cccc(Cl)c1F]

R₃ can be selected from B₁ can be a carbon centre and B₂ can be a carbon centre, wherein B₃ is a single bond between B₂ and the nitrogen atom to which R₁ is attached and B₁ is connected to B₂ via a single bond, or B₁ can be a nitrogen centre and B₂ can be a carbon centre wherein B₁ is connected to B₂ via a double bond; B₄ can be N(R₂), B₅ can be N, and B₆ can be C(R₃), wherein B₄ is connected to B₅ via a single bond and B₅ is connected to B₆ via a double bond, or B₄ can be C(R₂), Bs can be N, and B₆ can be N(R₃), wherein B₄ is connected to B₅ via a double bond and B₅ is connected to B₆ via a single bond; R₂ can be selected from —C(═O)—N(R₄)(R₅),

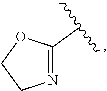 [SMILES: *C1=NCCO1]

—C(═O)—O—R₆,—CH₂—NH—S(═O)₂R₃₅,—CH₂—NH—C(═O) CH₃,—CH₂—O—R₁₆,—CH₂—OH, —CN, and wherein:
R₄ is H or small alkyl, and
R₅ is selected from alkyl, OH, —S(═O)₂-(small alkyl), —(CH₂)₁₋₃—(C═O)—OH, —(CH₂)₀₋₃-cycloalkyl^{b}, —(CH₂)₀₋₃-heterocycloalkyl, —(CH(alkyl)-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, —(C(H)(heteroaryl))-alkyl, —(C(H)(alkyl))—(C═O)—OH, and —(C(H)(heteroaryl))—(C═O)—OR_{28;}
or R₄ and R₅ together with the nitrogen atom to which they are attached can form a carbon-containing 4-, 5-, or 6-membered saturated heterocyclic ring, optionally containing an additional heteroatom selected from N, and O, which may be optionally mono- or di-substituted with substituents selected from oxo, alkyl, alkoxy, OH, halo and CF₃; R₆ is H, small alkyl, and —(CH₂)-phenyl;
R₁₆ is selected from H, small alkyl, —(CH₂)—(C═O)—OH, heterocycloalkyl, and —(CH₂)-heteroaryl; and R₃₅ is selected from H, small alkyl, and cyclopropyl; and

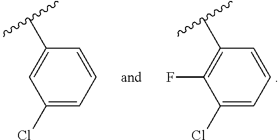 [SMILES: *c1cccc(Cl)c1 ; *c1cccc(Cl)c1F]

R₃ can be selected from R₂ can be selected from —C(═O)—N(R₄)(R₅), —C(═O)—O—R₆,—CH₂—NH—S(═O)₂R₃₅,—CH₂—NH—C(═O) CH₃,—CH₂—O—R₁₆,

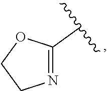 [SMILES: *C1=NCCO1]

—CH₂—OH, —CN, and wherein:
R₄ is H or small alkyl, and
R₅ is selected from alkyl, OH, —S(═O)₂-(small alkyl), —(CH₂)₁₋₃—(C═O)—OH, —(CH₂)₀₋₃-cycloalkyl′, —(CH₂)₀₋₃-heterocycloalkyl, —(CH(alkyl)-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, —(C(H)(heteroaryl))-alkyl, —(C(H)(alkyl))—(C═O)—OH, and —(C(H)(heteroaryl))—(C═O)—OR_{28;}
or R₄ and R₅ together with the nitrogen atom to which they are attached can form a carbon-containing 4-, 5-, or 6-membered saturated heterocyclic ring, optionally containing an additional heteroatom selected from N, and O, which may be optionally mono- or di-substituted with substituents selected from oxo, alkyl, alkoxy, OH, halo and CF₃; R₆ is H, small alkyl, and —(CH₂)-phenyl; R₁₆ is selected from H, small alkyl, —(CH₂)—(C═O)—OH, heterocycloalkyl, and —(CH₂)-heteroaryl; and
Ras is selected from H, small alkyl, and cyclopropyl; and

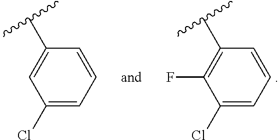 [SMILES: *c1cccc(Cl)c1 ; *c1cccc(Cl)c1F]

R₃ can be selected from B₁ can be a carbon centre and B₂ can be a carbon centre, wherein B₃ is a single bond between B₂ and the nitrogen atom to which R₁ is attached and B₁ is connected to B₂ via a single bond, or B₁ can be a nitrogen centre and B₂ can be a carbon centre wherein B₁ is connected to B₂ via a double bond;
R₁ can be:
(i) Formula (IIb),

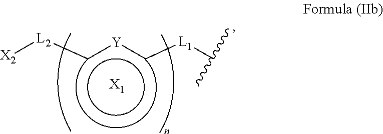

wherein:
n is 1;
L₁ is a linker selected from a single bond and —(CH₂)₁₋₃—;
Y is selected from C, N, O and S;
X₁ is phenyl or a 5- or 6-membered carbon-containing aromatic ring containing one, two or three ring members that are selected from N, S, and O;
wherein X₁ may be optionally substituted with 1 or 2 substituents independently selected from alkyl, cycloalkyl, alkoxy, OH, O(cyclopropyl), CF₃, and halo;
L₂ is a linker selected from a single bond, —(CH₂)₁₋₃—, and —C(═O)—; and
X₂ is either:
(a) —NR₂OR₂₁, wherein R₂₀ is selected from H, alkyl, and —(CH₂)₀₋₃-R_{22;}
wherein Rz1 is selected from H, alkyl, and —(CH₂)₀₋₃-R₂₃; wherein R₂₂ is selected from cycloalkyl, heterocycloalkyl, aryl, heteroaryl,

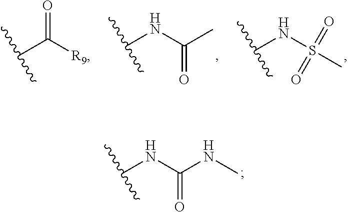

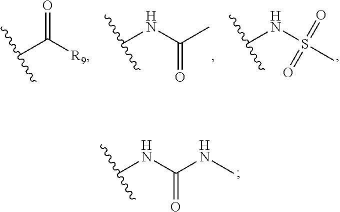

wherein R₂₃ is selected from cycloalkyl, heterocycloalkyl, aryl, heteroaryl, or
(b)₂-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O, and X₂ may be further optionally substituted with 1 or 2 substituents R₂₉ and R₃₀, wherein R₂₉ and R₃₀ are independently selected from alkyl, cycloalkyl, alkoxy, OH, oxo, CF₃, and halo; or
(ii) Formula (III) comprising an aromatic ring A fused to a non-aromatic ring A′,

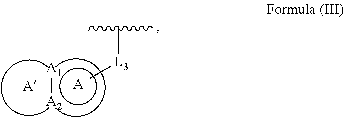

wherein:
L₃ is a single bond;
A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, O(cyclopropyl), CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R₇ is selected from alkyl, alkoxy, and —(CH₂)₀₋₃-R₈,

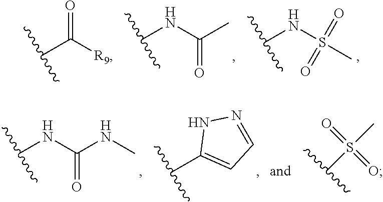

wherein R₈ is selected from cycloalkyl, heterocycloalkyl, wherein R₉ is selected from H, alkyl, OH, N(R₃₁)(R₃₂), and alkoxy;
wherein R₃₁ is H or small alkyl;
wherein R₃₂ is selected from H, small alkyl, and —(CH₂)₀₋₃heteroaryl;
wherein A′ may be optionally substituted with 1, 2, or 3 further substituents R₁₀, R₁₁, and R₁₇, wherein R₁₀, R₁₁, and R₁₇, are independently selected from alkyl, cycloalkyl, alkoxy, OH, oxo, CF₃, and halo, or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O, or wherein A′ may be optionally substituted with 1 or 2 further substituents R₄₃ and R₄₄ independently selected from alkyl, cycloalkyl, alkoxy, OH, oxo, CF₃, and halo, and two different carbon ring members within A′ are connected by a bridging —CH₂-group; and

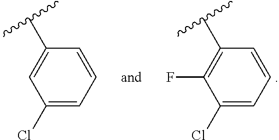 [SMILES: *c1cccc(Cl)c1 ; *c1cccc(Cl)c1F]

R₃ can be selected from B₄ can be N(R₂), B₅ can be N, and B₆ can be C(R₃), wherein B₄ is connected to Bs via a single bond and B₅ is connected to B₆ via a double bond, or B₄ can be C(R₂), Bs can be N, and B₆ can be N(R₃), wherein B₄ is connected to B₅ via a double bond and B₅ is connected to B₆ via a single bond;

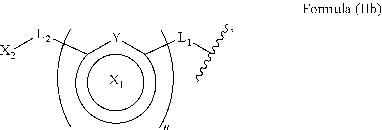

wherein:
n is 1;
L₁ is a linker selected from a single bond and —(CH₂)₁₋₃—;
Y is selected from C, N, O and S; X₁ is phenyl or a 5- or 6-membered carbon-containing aromatic ring containing one, two or three ring members that are selected from N, S, and O;
wherein X₁ may be optionally substituted with 1 or 2 substituents independently selected from alkyl, cycloalkyl, alkoxy, OH, O(cyclopropyl), CF₃, and halo; L₂ is a linker selected from a single bond, —(CH₂)₁₋₃—, and —C(═O)—; and X₂ is either:
(a) —NR₂OR₂₁, wherein R₂₀ is selected from H, alkyl, and —(CH₂)₀₋₃-R_{22;}
wherein R₂₁ is selected from H, alkyl, and —(CH₂)₀₋₃-R₂₃;

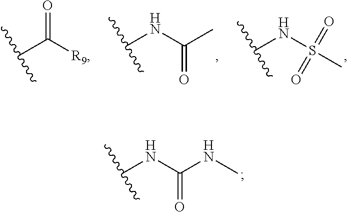

wherein R₂₂ is selected from cycloalkyl, heterocycloalkyl, aryl, heteroaryl,

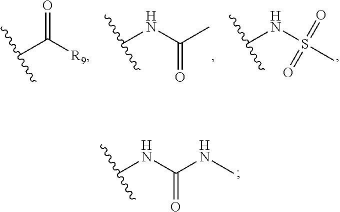

wherein R₂₃ is selected from cycloalkyl, heterocycloalkyl, aryl, heteroaryl, or
(b)₂-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O, and X₂ may be further optionally substituted with 1 or 2 substituents R₂₉ and R₃₀, wherein R₂₉ and R₃₀ are independently selected from alkyl, cycloalkyl, alkoxy, OH, oxo, CF₃, and halo; or
(ii) Formula (III) comprising an aromatic ring A fused to a non-aromatic ring A′,

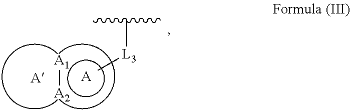

wherein:
L₃ is a single bond;
A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, O(cyclopropyl), CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R₇ is selected from alkyl, alkoxy, and —(CH₂)₀₋₃-R₈,

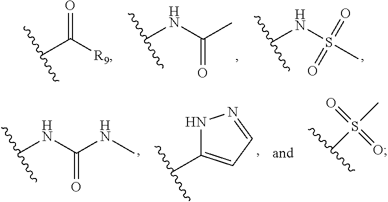

wherein R₈ is selected from cycloalkyl, heterocycloalkyl, wherein R₉ is selected from H, alkyl, OH, N(R₃₁)(R₃₂), and alkoxy;
wherein R₃₁ is H or small alkyl;
wherein R₃₂ is selected from H, small alkyl, and —(CH₂)₀₋₃heteroaryl;
wherein A′ may be optionally substituted with 1, 2, or 3 further substituents R₁₀, R₁₁, and R₁₇, wherein R₁₀, R₁₁, and R₁₇, are independently selected from alkyl, cycloalkyl, alkoxy, OH, oxo, CF₃, and halo, or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O, or wherein A′ may be optionally substituted with 1 or 2 further substituents R₄₃ and R₄₄ independently selected from alkyl, cycloalkyl, alkoxy, OH, oxo, CF₃, and halo, and two different carbon ring members within A′ are connected by a bridging —CH₂-group; and

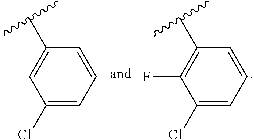 [SMILES: *c1cccc(Cl)c1 ; *c1cccc(Cl)c1F]

R₃ can be selected from B₁ can be a carbon centre and B₂ can be a carbon centre, wherein B₃ is a single bond between B₂ and the nitrogen atom to which R₁ is attached and B₁ is connected to B₂ via a single bond, or B₁ can be a nitrogen centre and B₂ can be a carbon centre wherein B₁ is connected to B₂ via a double bond; B₄ can be N(R₂), Bs can be N, and B₆ can be C(R₃), wherein B₄ is connected to B₅ via a single bond and B₅ is connected to B₆ via a double bond, or B₄ can be C(R₂), B₅ can be N, and B₆ can be N(R₃), wherein B₄ is connected to B₅ via a double bond and B₅ is connected to B₆ via a single bond;
R₁ can be:
(i) Formula (IIb),

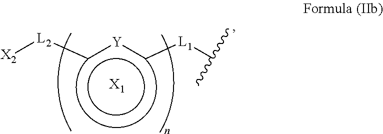

wherein:
n is 1;
L₁ is a linker selected from a single bond and —(CH₂)₁₋₃—;
Y is selected from C, N, O and S;
X₁ is phenyl or a 5- or 6-membered carbon-containing aromatic ring containing one, two or three
wherein X₁ may be optionally substituted with 1 or 2 substituents independently selected from alkyl, cycloalkyl, alkoxy, OH, O(cyclopropyl), CF₃, and halo; L₂ is a linker selected from a single bond, —(CH₂)₁₋₃—, and —C(═O)—; and X₂ is either:
(a) —NR₂OR₂₁, wherein R₂₀ is selected from H, alkyl, and —(CH₂)₀₋₃-R_{22;}
wherein R₂₁ is selected from H, alkyl, and —(CH₂)₀₋₃-R₂₃; wherein R₂₂ is selected from cycloalkyl, heterocycloalkyl, aryl, heteroaryl,

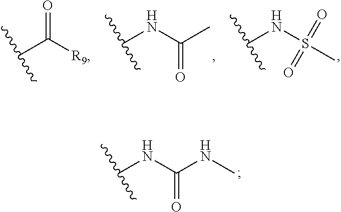

wherein R₂₃ is selected from cycloalkyl, heterocycloalkyl, aryl, heteroaryl,

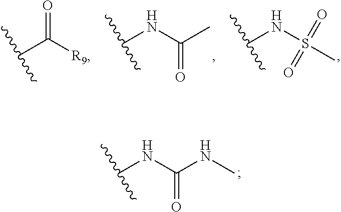

or
(b)₂-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O, and X₂ may be further optionally substituted with 1 or 2 substituents R₂₉ and R₃₀, wherein R₂₉ and R₃₀ are independently selected from alkyl, cycloalkyl, alkoxy, OH, oxo, CF₃, and halo; or

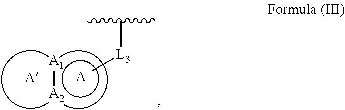

wherein:
L₃ is a single bond;
A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, O(cyclopropyl), CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R₇ is selected from alkyl, alkoxy, and —(CH₂)₀₋₃-R₈,

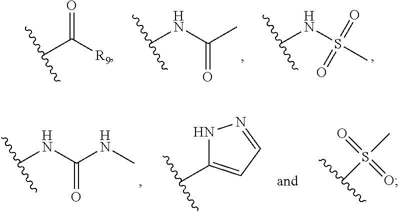

wherein R₈ is selected from cycloalkyl, heterocycloalkyl, wherein R₉ is selected from H, alkyl, OH, N(R₃₁)(R₃₂), and alkoxy;
wherein R₃₁ is H or small alkyl;
wherein R₃₂ is selected from H, small alkyl, and —(CH₂)₀₋₃heteroaryl;
wherein A′ may be optionally substituted with 1, 2, or 3 further substituents R₁₀, R₁₁, and R₁₇, wherein R₁₀, R₁₁, and R₁₇, are independently selected from alkyl, cycloalkyl, alkoxy, OH, oxo, CF₃, and halo, or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O, or wherein A′ may be optionally substituted with 1 or 2 further substituents R₄₃ and R₄₄ independently selected from alkyl, cycloalkyl, alkoxy, OH, oxo, CF₃, and halo, and two different carbon ring members within A′ are connected by a bridging —CH₂-group; and

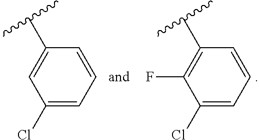 [SMILES: *c1cccc(Cl)c1 ; *c1cccc(Cl)c1F]

R₃ can be selected from B₁ can be a carbon centre and B₂ can be a carbon centre, wherein B₃ is a single bond between B₂ and the nitrogen atom to which R₁ is attached and B₁ is connected to B₂ via a single bond, or B₁ can be a nitrogen centre and B₂ can be a carbon centre wherein B₁ is connected to B₂ via a double bond; and R₂ can be selected from —C(═O)—N(R₄)(R₅), —C(═O)—O—R₆,—CH₂—NH—C(═O) CH₃,—CH₂—NH—S(═O)₂R₃₅,

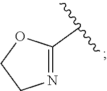 [SMILES: *C1=NCCO1]

—CH₂—O—R₁₆,—CH₂—OH, —CN, and
R₁ can be:
(i) Formula (IIb),

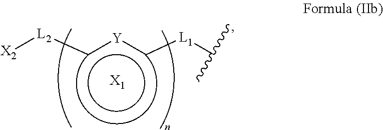

wherein:
n is 1;
L₁ is a linker selected from a single bond and —(CH₂)₁₋₃—;
Y is selected from C, N, O and S;
X₁ is phenyl or a 5- or 6-membered carbon-containing aromatic ring containing one, two or three ring members that are selected from N, S, and O;
wherein X₁ may be optionally substituted with 1 or 2 substituents independently selected from alkyl, cycloalkyl, alkoxy, OH, O(cyclopropyl), CF₃, and halo;
L₂ is a linker selected from a single bond, —(CH₂)₁₋₃—, and —C(═O)—; and
X₂ is either:
(a) —NR₂OR₂₁, wherein R₂₀ is selected from H, alkyl, and —(CH₂)₀₋₃-R_{22;}
wherein R₂₁ is selected from H, alkyl, and —(CH₂)₀₋₃-R₂₃;

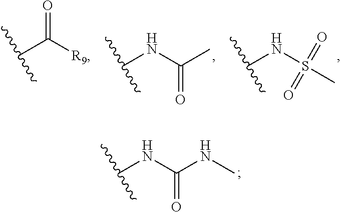

wherein R₂₂ is selected from cycloalkyl, heterocycloalkyl, aryl, heteroaryl,

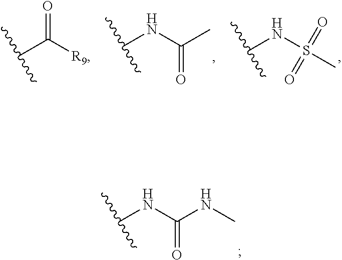

wherein R₂₃ is selected from cycloalkyl, heterocycloalkyl, aryl, heteroaryl, or
(b)₂-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O, and X₂ may be further optionally substituted with 1 or 2 substituents R₂₉ and R₃₀, wherein R₂₉ and R₃₀ are independently selected from alkyl, cycloalkyl, alkoxy, OH, oxo, CF₃, and halo; or
(ii) Formula (III) comprising an aromatic ring A fused to a non-aromatic ring A′,

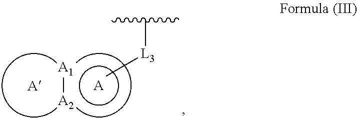

wherein:
L₃ is a single bond;
A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, O(cyclopropyl), CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R₇ is selected from alkyl, alkoxy, and —(CH₂)₀₋₃-R₈,

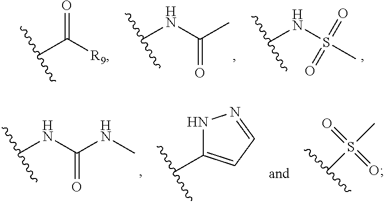

wherein R₈ is selected from cycloalkyl, heterocycloalkyl, wherein R₉ is selected from H, alkyl, OH, N(R₃₁)(R₃₂), and alkoxy;
wherein R₃₁ is H or small alkyl;
wherein R₃₂ is selected from H, small alkyl, and —(CH₂)₀₋₃heteroaryl;
wherein A′ may be optionally substituted with 1, 2, or 3 further substituents R₁₀, R₁₁, and R₁₇, wherein R₁₀, R₁₁, and R₁₇, are independently selected from alkyl, cycloalkyl, alkoxy, OH, oxo, CF₃, and halo, or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O, or wherein A′ may be optionally substituted with 1 or 2 further substituents R₄₃ and R₄₄ independently selected from alkyl, cycloalkyl, alkoxy, OH, oxo, CF₃, and halo, and two different carbon ring members within A′ are connected by a bridging —CH₂-group; and

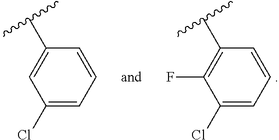 [SMILES: *c1cccc(Cl)c1 ; *c1cccc(Cl)c1F]

R₃ can be selected from B₄ can be N(R₂), B₅ can be N, and B₆ can be C(R₃), wherein B₄ is connected to Bs via a single bond and B₅ is connected to B₆ via a double bond, or B₄ can be C(R₂), B₅ can be N, and B₆ can be N(R₃), wherein B₄ is connected to B₅ via a double bond and B₅ is connected to B₆ via a single bond; R₂ can be selected from-C(═O)—N(R₄)(R₅), —C(═O)—O—R₆,—CH₂—NH—C(═O) CH₃,—CH₂—NH—S(═O)₂R₃₅,—CH₂—O—R₁₆,—CH₂—OH, —CN, and

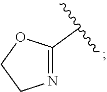 [SMILES: *C1=NCCO1]

R₁ can be:

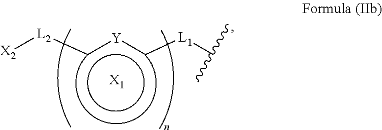

wherein:
n is 1;
L₁ is a linker selected from a single bond and —(CH₂)₁₋₃—;
Y is selected from C, N, O and S; X₁ is phenyl or a 5- or 6-membered carbon-containing aromatic ring containing one, two or three ring members that are selected from N, S, and O;
wherein X₁ may be optionally substituted with 1 or 2 substituents independently selected from alkyl, cycloalkyl, alkoxy, OH, O(cyclopropyl), CF₃, and halo; L₂ is a linker selected from a single bond, —(CH₂)₁₋₃—, and —C(═O)—; and
X₂ is either:
(a) —NR₂OR₂₁, wherein R₂₀ is selected from H, alkyl, and —(CH₂)₀₋₃-R_{22;}
wherein R₂₁ is selected from H, alkyl, and —(CH₂)₀₋₃-R₂₃;

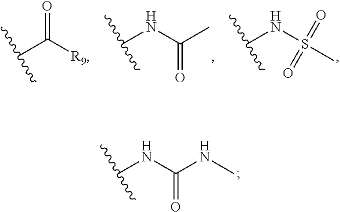

wherein R₂₂ is selected from cycloalkyl, heterocycloalkyl, aryl, heteroaryl,

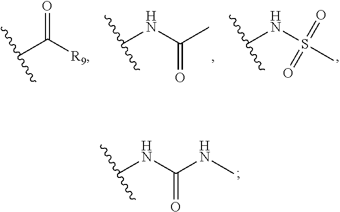

wherein R₂₃ is selected from cycloalkyl, heterocycloalkyl, aryl, heteroaryl, or
(b)₂-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O, and X₂ may be further optionally substituted with 1 or 2 substituents R₂₉ and R₃₀, wherein R₂₉ and R₃₀ are independently selected from alkyl, cycloalkyl, alkoxy, OH, oxo, CF₃, and halo; or
(ii) Formula (III) comprising an aromatic ring A fused to a non-aromatic ring A′,

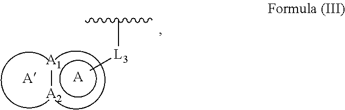

wherein:
L₃ is a single bond;
A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, O(cyclopropyl), CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R₇ is selected from alkyl, alkoxy, and —(CH₂)₀₋₃-R₈,

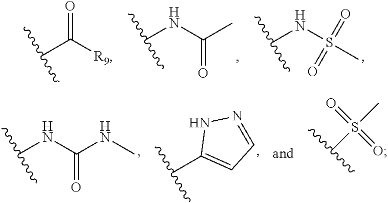

wherein R is selected from cycloalkyl, heterocycloalkyl, wherein R₉ is selected from H, alkyl, OH, N(R₃₁)(R₃₂), and alkoxy;
wherein R₃₁ is H or small alkyl;
wherein R₃₂ is selected from H, small alkyl, and —(CH₂)₀₋₃heteroaryl;
wherein A′ may be optionally substituted with 1, 2, or 3 further substituents R₁₀, R₁₁, and R₁₇, wherein R₁₀, R₁₁, and R₁₇, are independently selected from alkyl, cycloalkyl, alkoxy, OH, oxo, CF₃, and halo, or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O, or wherein A′ may be optionally substituted with 1 or 2 further substituents R₄₃ and R₄₄ independently selected from alkyl, cycloalkyl, alkoxy, OH, oxo, CF₃, and halo, and two different carbon ring members within A′ are connected by a bridging —CH₂-group; and

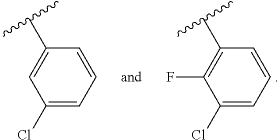 [SMILES: *c1cccc(Cl)c1 ; *c1cccc(Cl)c1F]

R₃ can be selected from B₁ can be a carbon centre and B₂ can be a carbon centre, wherein B₃ is a single bond between B₂ and the nitrogen atom to which R₁ is attached and B₁ is connected to B₂ via a single bond, or B₁ can be a nitrogen centre and B₂ can be a carbon centre wherein B₁ is connected to B₂ via a double bond; B₄ can be N(R₂), B₅ can be N, and B₆ can be C(R₃), wherein B₄ is connected to B₅ via a single bond and B₅ is connected to Be via a double bond, or B₄ can be C(R₂), B₅ can be N, and B₆ can be N(R₃), wherein B₄ is connected to B₅ via a double bond and B₅ is connected to B₆ via a single bond; R₂ can be selected from —C(═O)—N(R₄)(R₅),

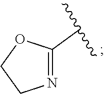 [SMILES: *C1=NCCO1]

—C(═O)—O—R₆,—CH₂—NH—S(═O)₂R₃₅,—CH₂—NH—C(═O) CH₃,—CH₂—O—R₁₆,—CH₂—OH, —CN, and
R₁ can be:
(i) Formula (IIb),

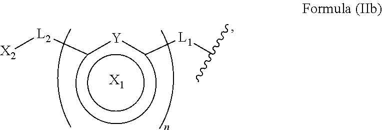

wherein:
n is 1;
L₁ is a linker selected from a single bond and —(CH₂)₁₋₃—;
Y is selected from C, N, O and S;
X₁ is phenyl or a 5- or 6-membered carbon-containing aromatic ring containing one, two or three ring members that are selected from N, S, and O;
wherein X₁ may be optionally substituted with 1 or 2 substituents independently selected from alkyl, cycloalkyl, alkoxy, OH, O(cyclopropyl), CF₃, and halo; L₂ is a linker selected from a single bond, —(CH₂)₁₋₃—, and —C(═O)—; and
X₂ is either:
(a) —NR₂OR₂₁, wherein R₂₀ is selected from H, alkyl, and —(CH₂)₀₋₃-R_{22;}
wherein R₂₁ is selected from H, alkyl, and —(CH₂)₀₋₃-R₂₃;

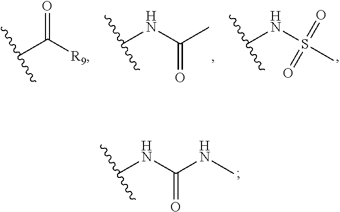

wherein R₂₂ is selected from cycloalkyl, heterocycloalkyl, aryl, heteroaryl,

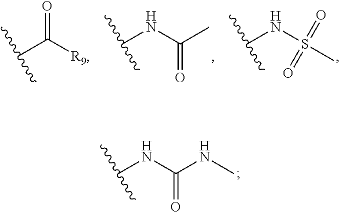

wherein R₂₃ is selected from cycloalkyl, heterocycloalkyl, aryl, heteroaryl, or
(b)₂-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O, and X₂ may be further optionally substituted with 1 or 2 substituents R₂₉ and R₃₀, wherein R₂₉ and R₃₀ are independently selected from alkyl, cycloalkyl, alkoxy, OH, oxo, CF₃, and halo; or

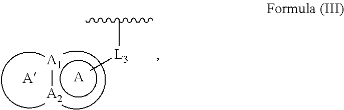

wherein:
L₃ is a single bond;
A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, O(cyclopropyl), CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R₇ is selected from alkyl, alkoxy, and —(CH₂)₀₋₃-R₈,

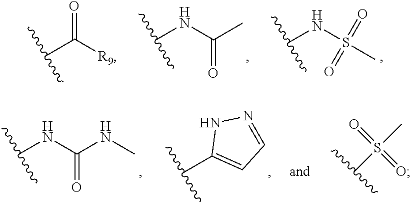

wherein R₈ is selected from cycloalkyl, heterocycloalkyl, wherein R₉ is selected from H, alkyl, OH, N(R₃₁)(R₃₂), and alkoxy;
wherein R₃₁ is H or small alkyl;
wherein R₃₂ is selected from H, small alkyl, and —(CH₂)₀₋₃heteroaryl;
wherein A′ may be optionally substituted with 1, 2, or 3 further substituents R₁₀, R₁₁, and R₁₇, wherein R₁₀, R₁₁, and R₁₇, are independently selected from alkyl, cycloalkyl, alkoxy, OH, oxo, CF₃, and halo, or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O, or wherein A′ may be optionally substituted with 1 or 2 further substituents R₄₃ and R₄₄ independently selected from alkyl, cycloalkyl, alkoxy, OH, oxo, CF₃, and halo, and two different carbon ring members within A′ are connected by a bridging —CH₂-group; and

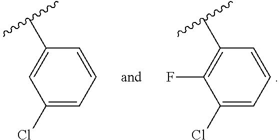 [SMILES: *c1cccc(Cl)c1 ; *c1cccc(Cl)c1F]

R₃ can be selected from R₂ can be selected from —C(═O)—N(R₄)(R₅), —C(═O)—O—R₆,—CH₂—NH—S(═O)₂R₃₅,—CH₂—NH—C(═O) CH₃,—CH₂—O—R₁₆,

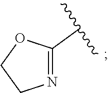 [SMILES: *C1=NCCO1]

—CH₂—OH, —CN, and
R₁ can be:
(i) Formula (IIb),

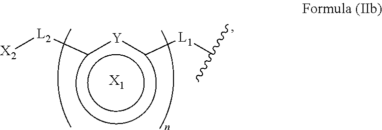

wherein:
n is 1;
L₁ is a linker selected from a single bond and —(CH₂)₁₋₃—;
Y is selected from C, N, O and S;
X₁ is phenyl or a 5- or 6-membered carbon-containing aromatic ring containing one, two or three ring members that are selected from N, S, and O;
wherein X₁ may be optionally substituted with 1 or 2 substituents independently selected from alkyl, cycloalkyl, alkoxy, OH, O(cyclopropyl), CF₃, and halo;
L₂ is a linker selected from a single bond, —(CH₂)₁₋₃—, and —C(═O)—; and
X₂ is either:
(a) —NR₂OR₂₁, wherein R₂₀ is selected from H, alkyl, and —(CH₂)₀₋₃-R_{22;}
wherein R₂₁ is selected from H, alkyl, and —(CH₂)₀₋₃-R₂₃;

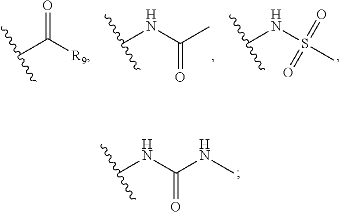

wherein R₂₂ is selected from cycloalkyl, heterocycloalkyl, aryl, heteroaryl,

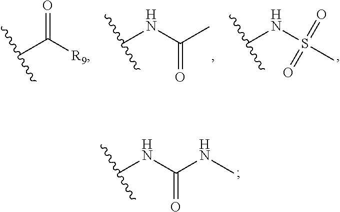

wherein R₂₃ is selected from cycloalkyl, heterocycloalkyl, aryl, heteroaryl, or
(b)₂-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O, and X₂ may be further optionally substituted with 1 or 2 substituents R₂₉ and R₃₀, wherein R₂₉ and R₃₀ are independently selected from alkyl, cycloalkyl, alkoxy, OH, oxo, CF₃, and halo; or
(ii) Formula (III) comprising an aromatic ring A fused to a non-aromatic ring A′,

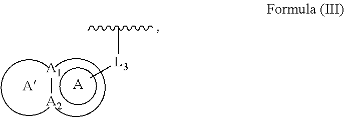

wherein:
L₃ is a single bond;
A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, O(cyclopropyl), CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R₇ is selected from alkyl, alkoxy, and —(CH₂)₀₋₃-R₈,

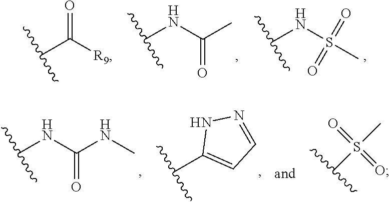

wherein R₈ is selected from cycloalkyl, heterocycloalkyl, wherein R₉ is selected from H, alkyl, OH, N(R₃₁)(R₃₂), and alkoxy;
wherein R₃₁ is H or small alkyl;
wherein R₃₂ is selected from H, small alkyl, and —(CH₂)₀₋₃heteroaryl;
wherein A′ may be optionally substituted with 1, 2, or 3 further substituents R₁₀, R₁₁, and R₁₇, wherein R₁₀, R₁₁, and R₁₇, are independently selected from alkyl, cycloalkyl, alkoxy, OH, oxo, CF₃, and halo, or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O, or wherein A′ may be optionally substituted with 1 or 2 further substituents R₄₃ and R₄₄ independently selected from alkyl, cycloalkyl, alkoxy, OH, oxo, CF₃, and halo, and two different carbon ring members within A′ are connected by a bridging —CH₂-group; and

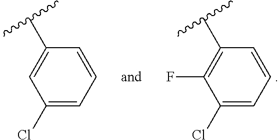 [SMILES: *c1cccc(Cl)c1 ; *c1cccc(Cl)c1F]

R₃ can be selected from B₁ can be a carbon centre and B₂ can be a carbon centre, wherein B₃ is a single bond between B₂ and the nitrogen atom to which R₁ is attached and B₁ is connected to B₂ via a single bond, or B₁ can be a nitrogen centre and B₂ can be a carbon centre wherein B₁ is connected to B₂ via a double bond; R₂ can be selected from —C(═O)—N(R₄)(R₅), —C(═O)—O—R₆,—CH₂—NH—C(═O) CH₃,—CH₂—NH—S(═O)₂R₃₅,—CH₂—O—R₁₆,—CH₂—OH, —CN, and

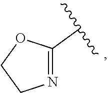

wherein:
R₄ is H or small alkyl, and
R₅ is selected from alkyl, OH, —S(═O)₂-(small alkyl), —(CH₂)₁₋₃—(C═O)—OH, —(CH₂)₀₋₃-cycloalkyl^{b}, —(CH₂)₀₋₃-heterocycloalkyl, —(CH(alkyl)-heterocycloalkyl,- (CH₂)₀₋₃-polycycloalkyl, —(CH₂)₀₋₃-aryl, —(C(H)(phenyl))-alkyl, —(CH₂)₀₋₃heteroaryl, —(C(H)(heteroaryl))-alkyl, —(C(H)(alkyl))—(C═O)—OH, and —(C(H)(heteroaryl))—(C═O)—OR_{28;}
or R₄ and R₅ together with the nitrogen atom to which they are attached can form a carbon-containing 4-, 5-, or 6-membered saturated heterocyclic ring, optionally containing an additional heteroatom selected from N, and O, which may be optionally mono- or di-substituted with substituents selected from oxo, alkyl, alkoxy, OH, halo and CF_{3;}
R₆ is H, small alkyl, and —(CH₂)-phenyl;
R₁₆ is selected from H, small alkyl, —(CH₂)—(C═O)—OH, heterocycloalkyl, and —(CH₂)-heteroaryl; and
R₃₅ is selected from H, small alkyl, and cyclopropyl;
R₁ can be:
(i) Formula (IIb),

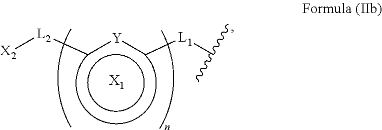

wherein:
n is 1;
L₁ is a linker selected from a single bond and —(CH₂)₁₋₃—;
Y is selected from C, N, O and S;
X₁ is phenyl or a 5- or 6-membered carbon-containing aromatic ring containing one, two or three ring members that are selected from N, S, and O;
wherein X₁ may be optionally substituted with 1 or 2 substituents independently selected from alkyl, cycloalkyl, alkoxy, OH, O(cyclopropyl), CF₃, and halo; L₂ is a linker selected from a single bond, —(CH₂)₁₋₃—, and —C(═O)—; and X₂ is either:
(a) NR₂OR₂₁, wherein R₂₀ is selected from H, alkyl, and —(CH₂)₀₋₃-R_{22;}
wherein R₂₁ is selected from H, alkyl, and —(CH₂)₀₋₃-R₂₃;

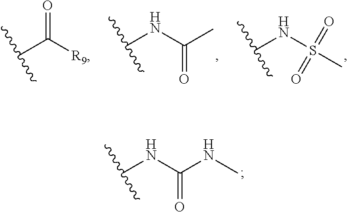

wherein R₂₂ is selected from cycloalkyl, heterocycloalkyl, aryl, heteroaryl,

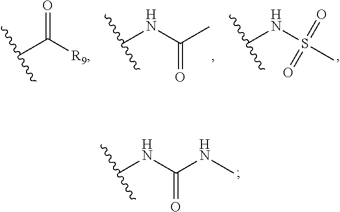

wherein R₂₃ is selected from cycloalkyl, heterocycloalkyl, aryl, heteroaryl, or
(b)₂-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O, and X₂ may be further optionally substituted with 1 or 2 substituents R₂₉ and R₃₀, wherein R₂₉ and R₃₀ are independently selected from alkyl, cycloalkyl, alkoxy, OH, oxo, CF₃, and halo; or
(ii) Formula (III) comprising an aromatic ring A fused to a non-aromatic ring A′,

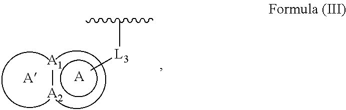

wherein:
L₃ is a single bond;
A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, O(cyclopropyl), CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R₇ is selected from alkyl, alkoxy, and —(CH₂)₀₋₃-R₈,

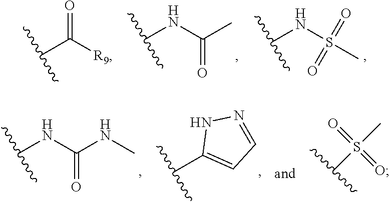

wherein R₈ is selected from cycloalkyl, heterocycloalkyl, wherein R₉ is selected from H, alkyl, OH, N(R₃₁)(R₃₂), and alkoxy;
wherein R₃₁ is H or small alkyl;
wherein R₃₂ is selected from H, small alkyl, and —(CH₂)₀₋₃heteroaryl;
wherein A′ may be optionally substituted with 1, 2, or 3 further substituents R₁₀, R₁₁, and R₁₇, wherein R₁₀, R₁₁, and R₁₇, are independently selected from alkyl, cycloalkyl, alkoxy, OH, oxo, CF₃, and halo, or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O, or wherein A′ may be optionally substituted with 1 or 2 further substituents R₄₃ and R₄₄ independently selected from alkyl, cycloalkyl, alkoxy, OH, oxo, CF₃, and halo, and two different carbon ring members within A′ are connected by a bridging —CH₂-group; and

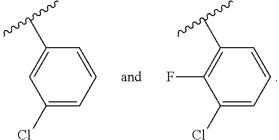 [SMILES: *c1cccc(Cl)c1 ; *c1cccc(Cl)c1F]

R₃ can be selected from B₄ can be N(R₂), B₅ can be N, and B₆ can be C(R₃), wherein B₄ is connected to Bs via a single bond and B₅ is connected to B₆ via a double bond, or B₄ can be C(R₂), B₅ can be N, and B₆ can be N(R₃), wherein B₄ is connected to B₅ via a double bond and B₅ is connected to B₆ via a single bond; R₂ can be selected from-C(═O)—N(R₄)(R₅), —C(═O)—O—R₆,—CH₂—NH—C(═O) CH₃,—CH₂—NH—S(═O)₂R₃₅,—CH₂—O—R₁₆,—CH₂—OH, —CN, and

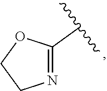

wherein:
R₄ is H or small alkyl, and
R₅ is selected from alkyl, OH, —S(═O)₂-(small alkyl), —(CH₂)₁₋₃—(C═O)—OH, —(CH₂)₀₋₃-cycloalkyl^{b}, —(CH₂)₀₋₃-heterocycloalkyl, —(CH(alkyl))-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, —(C(H)(heteroaryl))-alkyl, —(C(H)(alkyl))—(C═O)—OH, and —(C(H)(heteroaryl))—(C═O)—OR_{28;}
or R₄ and R₅ together with the nitrogen atom to which they are attached can form a carbon-containing 4-, 5-, or 6-membered saturated heterocyclic ring, optionally containing an additional heteroatom selected from N, and O, which may be optionally mono- or di-substituted with substituents selected from oxo, alkyl, alkoxy, OH, halo and CF_{3;}
R₆ is H, small alkyl, and —(CH₂)-phenyl;
R₁₆ is selected from H, small alkyl, —(CH₂)—(C═O)—OH, heterocycloalkyl, and —(CH₂)-heteroaryl; and R₃₅ is selected from H, small alkyl, and cyclopropyl;
R₁ can be:
(i) Formula (IIb),

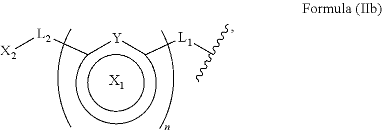

wherein:
n is 1;
L₁ is a linker selected from a single bond and —(CH₂)₁₋₃—;
Y is selected from C, N, O and S;
X₁ is phenyl or a 5- or 6-membered carbon-containing aromatic ring containing one, two or three ring members that are selected from N, S, and O;
wherein X₁ may be optionally substituted with 1 or 2 substituents independently selected from alkyl, cycloalkyl, alkoxy, OH, O(cyclopropyl), CF₃, and halo;
L₂ is a linker selected from a single bond, —(CH₂)₁₋₃—, and —C(═O)—; and
X₂ is either:
(a) —NR₂OR₂₁, wherein R₂₀ is selected from H, alkyl, and —(CH₂)₀₋₃-R_{22;}
wherein R₂₁ is selected from H, alkyl, and —(CH₂)₀₋₃-R₂₃;

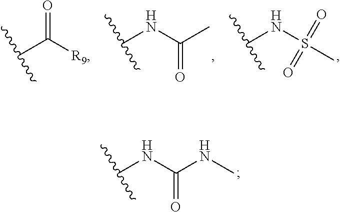

wherein R₂₂ is selected from cycloalkyl, heterocycloalkyl, aryl, heteroaryl,

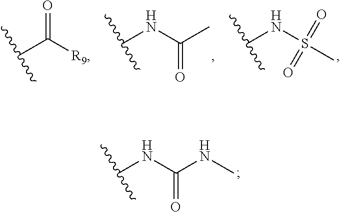

wherein R₂₃ is selected from cycloalkyl, heterocycloalkyl, aryl, heteroaryl, or
(b)₂-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected from alkyl, cycloalkyl, alkoxy, OH, Oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O, and X₂ may be further optionally substituted with 1 or 2 substituents R₂₉ and R₃₀, wherein R₂₉ and R₃₀ are independently selected from alkyl, cycloalkyl, alkoxy, OH, oxo, CF₃, and halo; or
(ii) Formula (III) comprising an aromatic ring A fused to a non-aromatic ring A′,

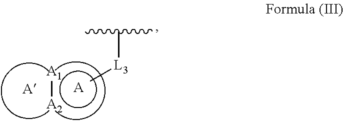

wherein:
L₃ is a single bond;
A₁ and A₂ are both carbon;
A is phenyl or a 5- or 6-membered carbon-containing aromatic ring containing one, two or three
wherein A may be optionally substituted with 1 or 2 substituents independently selected from alkyl, cycloalkyl, alkoxy, OH, O(cyclopropyl), CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R₇ is selected from alkyl, alkoxy, and —(CH₂)₀₋₃-R₈,

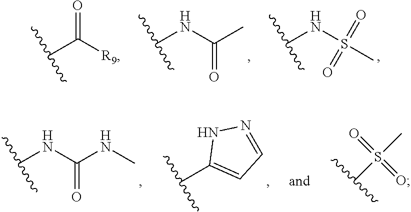

wherein R₈ is selected from cycloalkyl, heterocycloalkyl, wherein R₉ is selected from H, alkyl, OH, N(R₃₁)(R₃₂), and alkoxy;
wherein R₃₁ is H or small alkyl;
wherein R₃₂ is selected from H, small alkyl, and —(CH₂)₀₋₃heteroaryl;
wherein A′ may be optionally substituted with 1, 2, or 3 further substituents R₁₀, R₁₁, and R₁₇, wherein R₁₀, R₁₁, and R₁₇, are independently selected from alkyl, cycloalkyl, alkoxy, OH, oxo, CF₃, and halo, or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O, or wherein A′ may be optionally substituted with 1 or 2 further substituents R₄₃ and R₄₄ independently selected from alkyl, cycloalkyl, alkoxy, OH, oxo, CF₃, and halo, and two different carbon ring members within A′ are connected by a bridging —CH₂-group; and

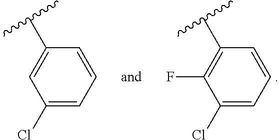 [SMILES: *c1cccc(Cl)c1 ; *c1cccc(Cl)c1F]

R₃ can be selected from B₁ can be a carbon centre and B₂ can be a carbon centre, wherein B₃ is a single bond between B₂ and the nitrogen atom to which R₁ is attached and B₁ is connected to B₂ via a single bond, or B₁ can be a nitrogen centre and B₂ can be a carbon centre wherein B₁ is connected to B₂ via a double bond; B₄ can be N(R₂), B₅ can be N, and B₆ can be C(R₃), wherein B₄ is connected to B₅ via a single bond and B₅ is connected to B₆ via a double bond, or B₄ can be C(R₂), B₅ can be N, and B₆ can be N(R₃), wherein B₄ is connected to B₅ via a double bond and B₅ is connected to B₆ via a single bond; R₂ can be selected from —C(═O)—N(R₄)(R₅),

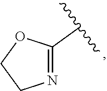 [SMILES: *C1=NCCO1]

—C(═O)—O—R₆,—CH₂—NH—S(═O)₂R₃₅,—CH₂—NH—C(═O) CH₃,—CH₂—O—R₁₆,—CH₂—OH, —CN, and wherein:
R₄ is H or small alkyl, and
R₅ is selected from alkyl, OH, —S(═O)₂-(small alkyl), —(CH₂)₁₋₃—(C═O)—OH, —(CH₂)₀₋₃-cycloalkyl^{b}, —(CH₂)₀₋₃-heterocycloalkyl, —(CH(alkyl)-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, —(C(H)(heteroaryl))-alkyl, —(C(H)(alkyl))—(C═O)—OH, and —(C(H)(heteroaryl))—(C═O)—OR_{28;}
or R₄ and R₅ together with the nitrogen atom to which they are attached can form a carbon-containing 4-, 5-, or 6-membered saturated heterocyclic ring, optionally containing an additional heteroatom selected from N, and O, which may be optionally mono- or di-substituted with substituents selected from oxo, alkyl, alkoxy, OH, halo and CF₃; R₆ is H, small alkyl, and —(CH₂)-phenyl;
R₁₆ is selected from H, small alkyl, —(CH₂)—(C═O)—OH, heterocycloalkyl, and —(CH₂)-heteroaryl; and R₃₅ is selected from H, small alkyl, and cyclopropyl;
R₁ can be:
(i) Formula (IIb),

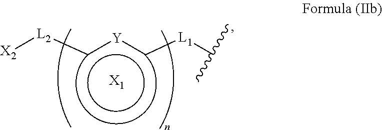

wherein:
n is 1;
L₁ is a linker selected from a single bond and —(CH₂)₁₋₃—;
Y is selected from C, N, O and S; X₁ is phenyl or a 5- or 6-membered carbon-containing aromatic ring containing one, two or three ring members that are selected from N, S, and O;
wherein X₁ may be optionally substituted with 1 or 2 substituents independently selected from alkyl, cycloalkyl, alkoxy, OH, O(cyclopropyl), CF₃, and halo; L₂ is a linker selected from a single bond, —(CH₂)₁₋₃—, and —C(═O)—; and
X₂ is either:
(a) —NR₂OR₂₁, wherein R₂₀ is selected from H, alkyl, and —(CH₂)₀₋₃-R_{22;}
wherein R₂₁ is selected from H, alkyl, and —(CH₂)₀₋₃-R₂₃;

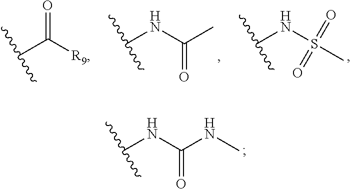

wherein R₂₂ is selected from cycloalkyl, heterocycloalkyl, aryl, heteroaryl,

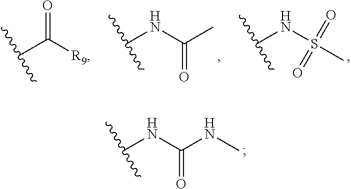

wherein R₂₃ is selected from cycloalkyl, heterocycloalkyl, aryl, heteroaryl, or
(b)₂-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O, and X₂ may be further optionally substituted with 1 or 2 substituents R₂₉ and R₃₀, wherein R₂₉ and R₃₀ are independently selected from alkyl, cycloalkyl, alkoxy, OH, oxo, CF₃, and halo; or
(ii) Formula (III) comprising an aromatic ring A fused to a non-aromatic ring A′,

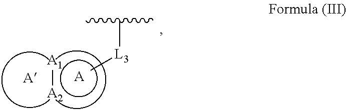

wherein:
L₃ is a single bond;
A₁ and Az are both carbon;
A is phenyl or a 5- or 6-membered carbon-containing aromatic ring containing one, two or three
wherein A may be optionally substituted with 1 or 2 substituents independently selected from alkyl, cycloalkyl, alkoxy, OH, O(cyclopropyl), CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R₇ is selected from alkyl, alkoxy, and —(CH₂)₀₋₃-R₈,

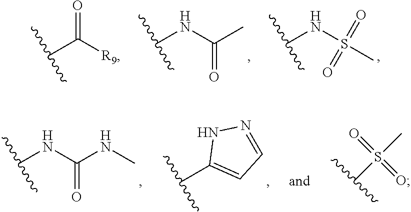

wherein R₈ is selected from cycloalkyl, heterocycloalkyl, wherein R₉ is selected from H, alkyl, OH, N(R₃₁)(R₃₂), and alkoxy;
wherein R₃₁ is H or small alkyl;
wherein R₃₂ is selected from H, small alkyl, and —(CH₂)₀₋₃heteroaryl;
wherein A′ may be optionally substituted with 1, 2, or 3 further substituents R₁₀, R₁₁, and R₁₇, wherein R₁₀, R₁₁, and R₁₇, are independently selected from alkyl, cycloalkyl, alkoxy, OH, oxo, CF₃, and halo, or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O, or wherein A′ may be optionally substituted with 1 or 2 further substituents R₄₃ and R₄₄ independently selected from alkyl, cycloalkyl, alkoxy, OH, oxo, CF₃, and halo, and two different carbon ring members within A′ are connected by a bridging —CH₂-group; and

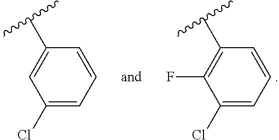 [SMILES: *c1cccc(Cl)c1 ; *c1cccc(Cl)c1F]

R₃ can be selected from R₂ can be selected from —C(═O)—N(R₄)(R₅), —C(═O)—O—R₆,—CH₂—NH—S(═O)₂R₃₅,—CH₂—NH—C(═O) CH₃,—CH₂—O—R₁₆,

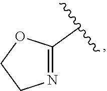 [SMILES: *C1=NCCO1]

—CH₂—OH, —CN, and wherein:
R₄ is H or small alkyl, and
R₅ is selected from alkyl, OH, —S(═O)₂-(small alkyl), —(CH₂)₁₋₃—(C═O)—OH, —(CH₂)₀₋₃-cycloalkyl^{b}, —(CH₂)₀₋₃-heterocycloalkyl, —(CH(alkyl)-heterocycloalkyl, —(CH₂)₀₋₃-polycycloalkyl, —(CH₂)₀₋₃-aryl, —(C(H)(phenyl)-alkyl, —(CH₂)₀₋₃heteroaryl, —(C(H)(heteroaryl))-alkyl, —(C(H)(alkyl))—(C═O)—OH, and —(C(H)(heteroaryl))—(C═O)—OR_{28;}
or R₄ and R₅ together with the nitrogen atom to which they are attached can form a carbon-containing 4-, 5-, or 6-membered saturated heterocyclic ring, optionally containing an additional heteroatom selected from N, and O, which may be optionally mono- or di-substituted with substituents selected from oxo, alkyl, alkoxy, OH, halo and CF_{3;}
R₆ is H, small alkyl, and —(CH₂)-phenyl;
R₁₆ is selected from H, small alkyl, —(CH₂)—(C═O)—OH, heterocycloalkyl, and —(CH₂)-heteroaryl; and R₃₅ is selected from H, small alkyl, and cyclopropyl;
R₁ can be:
(i) Formula (IIb),

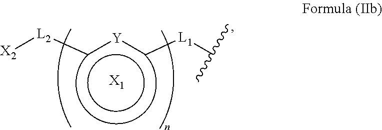

wherein:
n is 1;
L₁ is a linker selected from a single bond and —(CH₂)₁₋₃—;
Y is selected from C, N, O and S;
X₁ is phenyl or a 5- or 6-membered carbon-containing aromatic ring containing one, two or three ring members that are selected from N, S, and O;
wherein X₁ may be optionally substituted with 1 or 2 substituents independently selected from alkyl, cycloalkyl, alkoxy, OH, O(cyclopropyl), CF₃, and halo;
L₂ is a linker selected from a single bond, —(CH₂)₁₋₃—, and —C(═O)—; and
X₂ is either:
(a) —NR₂OR₂₁, wherein R₂₀ is selected from H, alkyl, and —(CH₂)₀₋₃-R_{22;}
wherein R₂₁ is selected from H, alkyl, and —(CH₂)₀₋₃-R₂₃;

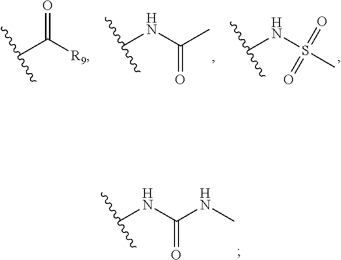

wherein R₂₂ is selected from cycloalkyl, heterocycloalkyl, aryl, heteroaryl,

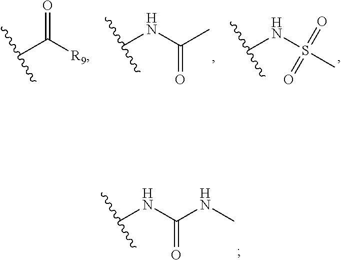

wherein R₂₃ is selected from cycloalkyl, heterocycloalkyl, aryl, heteroaryl, or
(b)₂-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected from alkyl, cycloalkyl, alkoxy, OH, Oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O, and X₂ may be further optionally substituted with 1 or 2 substituents R₂₉ and R₃₀, wherein R₂₉ and R₃₀ are independently selected from alkyl, cycloalkyl, alkoxy, OH, oxo, CF₃, and halo; or
(ii) Formula (III) comprising an aromatic ring A fused to a non-aromatic ring A′,

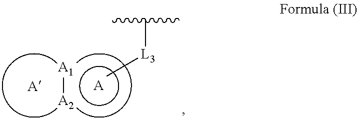

wherein:
L₃ is a single bond;
A₁ and A₂ are both carbon;
A is phenyl or a 5- or 6-membered carbon-containing aromatic ring containing one, two or three
wherein A may be optionally substituted with 1 or 2 substituents independently selected from alkyl, cycloalkyl, alkoxy, OH, O(cyclopropyl), CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R₇ is selected from alkyl, alkoxy, and —(CH₂)₀₋₃-R₈,

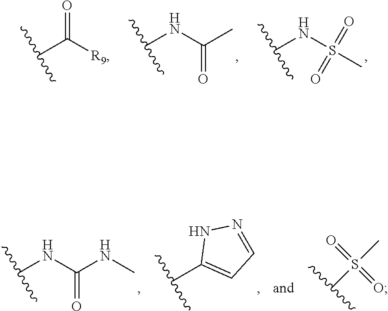

wherein R₈ is selected from cycloalkyl, heterocycloalkyl, wherein R₉ is selected from H, alkyl, OH, N(R₃₁)(R₃₂), and alkoxy;
wherein R₃₁ is H or small alkyl;
wherein R₃₂ is selected from H, small alkyl, and —(CH₂)₀₋₃heteroaryl;
wherein A′ may be optionally substituted with 1, 2, or 3 further substituents R₁₀, R₁₁, and R₁₇, wherein R₁₀, R₁₁, and R₁₇, are independently selected from alkyl, cycloalkyl, alkoxy, OH, oxo, CF₃, and halo, or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O, or wherein A′ may be optionally substituted with 1 or 2 further substituents R₄₃ and R₄₄ independently selected from alkyl, cycloalkyl, alkoxy, OH, oxo, CF₃, and halo, and two different carbon ring members within A′ are connected by a bridging —CH₂-group; and

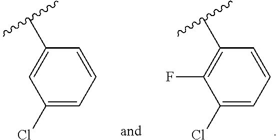 [SMILES: *c1cccc(Cl)c1 ; *c1cccc(Cl)c1F]

R₃ can be selected from

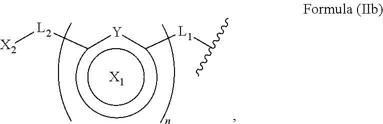

wherein:
n is 1;
L₁ is a linker selected from a single bond and —(CH₂)₁₋₃—;
Y is selected from C, N, O and S; X₁ is phenyl or a 5- or 6-membered carbon-containing aromatic ring containing one, two or three ring members that are selected from N, S, and O;
wherein X₁ may be optionally substituted with 1 or 2 substituents independently selected from alkyl, cycloalkyl, alkoxy, OH, O(cyclopropyl), CF₃, and halo; L₂ is a linker selected from a single bond, —(CH₂)₁₋₃—, and —C(═O)—; and
X₂ is either:
(a) —NR₂OR₂₁, wherein R₂₀ is selected from H, alkyl, and —(CH₂)₀₋₃-R_{22;}
wherein R₂₁ is selected from H, alkyl, and —(CH₂)₀₋₃-R₂₃;

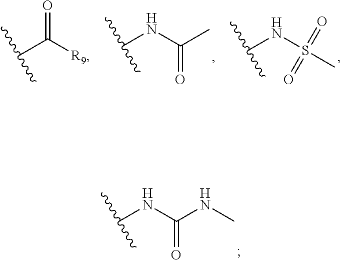

wherein R₂₂ is selected from cycloalkyl, heterocycloalkyl, aryl, heteroaryl,

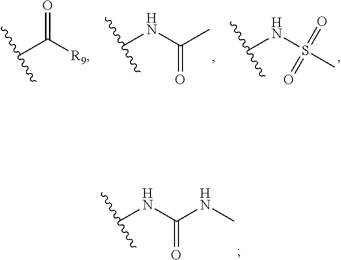

wherein R₂₃ is selected from cycloalkyl, heterocycloalkyl, aryl, heteroaryl, or
(b)₂-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected from alkyl, cycloalkyl, alkoxy, OH, Oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O, and X₂ may be further optionally substituted with 1 or 2 substituents R₂₉ and R₃₀, wherein R₂₉ and R₃₀ are independently selected from alkyl, cycloalkyl, alkoxy, OH, oxo, CF₃, and halo; or
(ii) Formula (III) comprising an aromatic ring A fused to a non-aromatic ring A′,

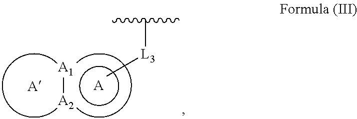

wherein:
L₃ is a single bond;
A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, O(cyclopropyl), CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R, is selected from alkyl, alkoxy, and —(CH₂)₀₋₃-R₈,

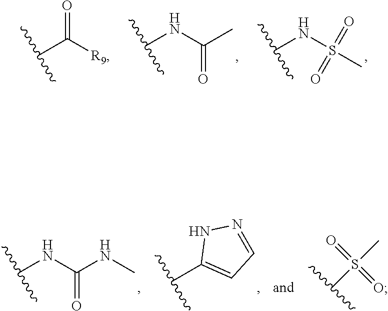

wherein R₈ is selected from cycloalkyl, heterocycloalkyl, wherein R₉ is selected from H, alkyl, OH, N(R₃₁)(R₃₂), and alkoxy;
wherein R₃₁ is H or small alkyl;
wherein R₃₂ is selected from H, small alkyl, and —(CH₂)₀₋₃heteroaryl;
wherein A′ may be optionally substituted with 1, 2, or 3 further substituents R₁₀, R₁₁, and R₁₇, wherein R₁₀, R₁₁, and R₁₇, are independently selected from alkyl, cycloalkyl, alkoxy, OH, oxo, CF₃, and halo, or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O, or wherein A′ may be optionally substituted with 1 or 2 further substituents R₄₃ and R₄₄ independently selected from alkyl, cycloalkyl, alkoxy, OH, oxo, CF₃, and halo, and two different carbon ring members within A′ are connected by a bridging —CH₂-group; and

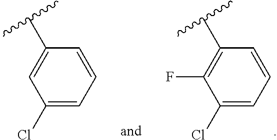 [SMILES: *c1cccc(Cl)c1 ; *c1cccc(Cl)c1F]

R₃ can be selected from B₁ can be a carbon centre and B₂ can be a carbon centre, wherein B₃ is a single bond between B₂ and the nitrogen atom to which R₁ is attached and B₁ is connected to B₂ via a single bond, or B₁ can be a nitrogen centre and B₂ can be a carbon centre wherein B₁ is connected to B₂ via a double bond; and R₃ can be substituted with one or two substituents independently selected from small alkyl, small alkoxy, CF₃, halo, —C(═O)—NH₂, and CN.
B₄ can be N(R₂), B₅ can be N, and B₆ can be C(R₃), wherein B₄ is connected to Bs via a single bond and B₅ is connected to B₆ via a double bond, or B₄ can be C(R₂), B₅ can be N, and B₆ can be N(R₃), wherein B₄ is connected to B₅ via a double bond and B₅ is connected to B₆ via a single bond; and R₃ can be substituted with one or two substituents independently selected from small alkyl, small alkoxy, CF₃, halo, —C(═O)—NH₂, and CN.
B₁ can be a carbon centre and B₂ can be a carbon centre, wherein B₃ is a single bond between B₂ and the nitrogen atom to which R₁ is attached and B₁ is connected to B₂ via a single bond, or B₁ can be a nitrogen centre and B₂ can be a carbon centre wherein B₁ is connected to B₂ via a double bond; B₄ can be N(R₂), B₅ can be N, and B₆ can be C(R₃), wherein B₄ is connected to B₅ via a single bond and B₅ is connected to B₆ via a double bond, or B₄ can be C(R₂), B₅ can be N, and B₆ can be N(R₃), wherein B₄ is connected to B₅ via a double bond and B₅ is connected to B₆ via a single bond; and R₃ can be phenyl substituted with one or two substituents independently selected from small alkyl, small alkoxy, CF₃, halo, —C(═O)—NH₂, and CN.
R₂ can be selected from —C(═O)—N(R₄)(R₅), —C(═O)—O—R₆,—CH₂—NH—C(═O) CH₃,—CH₂—NH—S(═O)₂(cyclopropyl),

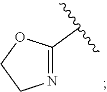 [SMILES: *C1=NCCO1]

—CH₂—O—R₁₆,—CH₂—OH, —CN, and and R₃ can be substituted with one or two substituents independently selected from small alkyl, small alkoxy, CF₃, halo, —C(═O)—NH₂, and CN.
B₁ can be a carbon centre and B₂ can be a carbon centre, wherein B₃ is a single bond between B₂ and the nitrogen atom to which R₁ is attached and B₁ is connected to B₂ via a single bond, or B₁ can be a nitrogen centre and B₂ can be a carbon centre wherein B₁ is connected to B₂ via a double bond; R₂ can be selected from —C(═O)—N(R₄)(R₅), —C(═O)—O—R₆,—CH₂—NH—C(═O) CH₃,—CH₂—NH—S(═O)₂(cyclopropyl),

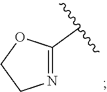 [SMILES: *C1=NCCO1]

—CH₂—O—R₁₆,—CH₂—OH, —CN, and; and R₃ can be substituted with one or two substituents independently selected from small alkyl, small alkoxy, CF₃, halo, —C(═O)—NH₂, and CN.
B₄ can be N(R₂), B₅ can be N, and B₆ can be C(R₃), wherein B₄ is connected to B₅ via a single bond and B₅ is connected to B₆ via a double bond, or B₄ can be C(R₂), B₅ can be N, and B₆ can be N(R₃), wherein B₄ is connected to B₅ via a double bond and B₅ is connected to B₆ via a single bond; R₂ can be selected from —C(═O)—N(R₄)(R₅), —C(═O)—O—R₆,—CH₂—NH—C(═O) CH₃,—CH₂—NH—S(═O)₂(cyclopropyl),—CH₂—O—R₁₆,—CH₂—OH,

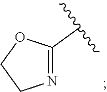 [SMILES: *C1=NCCO1]

—CN, and and R₃ can be substituted with one or two substituents independently selected from small alkyl, small alkoxy, CF₃, halo, —C(═O)—NH₂, and CN.
B₁ can be a carbon centre and B₂ can be a carbon centre, wherein B₃ is a single bond between B₂ and the nitrogen atom to which R₁ is attached and B₁ is connected to B₂ via a single bond, or B₁ can be a nitrogen centre and B₂ can be a carbon centre wherein B₁ is connected to B₂ via a double bond; B₄ can be N(R₂), B₅ can be N, and B₆ can be C(R₃), wherein B₄ is connected to B₅ via a single bond and B₅ is connected to B₆ via a double bond, or B₄ can be C(R₂), B₅ can be N, and B₆ can be N(R₃), wherein B₄ is connected to B₅ via a double bond and B₅ is connected to B₆ via a single bond; R₂ can be selected from —C(═O)—N(R₄)(R₅),

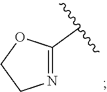 [SMILES: *C1=NCCO1]

—C(═O)—O—R₆,—CH₂—NH—S(═O)₂(cyclopropyl),—CH₂—NH—C(═O) CH₃,—CH₂—O—R₁₆,—CH₂—OH, —CN, and and R₃ can be substituted with one or two substituents independently selected from small alkyl, small alkoxy, CF₃, halo, —C(═O)—NH₂, and CN.
B₁ can be a carbon centre and B₂ can be a carbon centre, wherein B₃ is a single bond between B₂ and the nitrogen atom to which R₁ is attached and B₁ is connected to B₂ via a single bond, or B₁ can be a nitrogen centre and B₂ can be a carbon centre wherein B₁ is connected to B₂ via a double bond; R₂ can be selected from —C(═O)—N(R₄)(R₅), —C(═O)—O—R₆,—CH₂—NH—C(═O) CH₃,—CH₂—NH—S(═O)₂(cyclopropyl),—CH₂—O—R₁₆,—CH₂—OH,

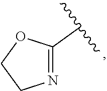 [SMILES: *C1=NCCO1]

—CN, and wherein:
R₄ is H or small alkyl, and
R₅ is selected from alkyl, OH, —S(═O)₂-(small alkyl), —(CH₂)₁₋₃—(C═O)—OH, —(CH₂)₀₋₃-cycloalkyl^{a}, —(CH₂)₀₋₃-heterocycloalkyl, —(CH(alkyl))-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, —(C(H)(heteroaryl))-alkyl, —(C(H)(alkyl))—(C═O)—OH, and —(C(H)(heteroaryl))—(C═O)—OR_{28;}
or R₄ and R₅ together with the nitrogen atom to which they are attached can form a carbon-containing 4-, 5-, or 6-membered saturated heterocyclic ring, optionally containing an additional heteroatom selected from N, and O, which may be optionally mono- or di-substituted with substituents selected from oxo, alkyl, alkoxy, OH, halo and CF₃; and
R₆ is H, small alkyl, and —(CH₂)-phenyl; and
R₃ can be substituted with one or two substituents independently selected from small alkyl, small alkoxy, CF₃, halo, —C(═O)—NH₂, and CN.
B₄ can be N(R₂), B₅ can be N, and B₆ can be C(R₃), wherein B₄ is connected to B₅ via a single bond and B₅ is connected to B₆ via a double bond, or B₄ can be C(R₂), B₅ can be N, and B₆ can be N(R₃), wherein B₄ is connected to B₅ via a double bond and B₅ is connected to B₆ via a single bond; R₂ can be selected from —C(═O)—N(R₄)(R₅), —C(═O)—O—R₆,—CH₂—NH—C(═O) CH₃,—CH₂—NH—S(═O)₂(cyclopropyl),—CH₂—O—R₁₆,—CH₂—OH,

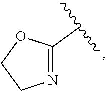 [SMILES: *C1=NCCO1]

—CN, and wherein: R₄ is H or small alkyl, and R₅ is selected from alkyl, OH, —S(═O)₂-(small alkyl), —(CH₂)₁₋₃—(C═O)—OH, —(CH₂)₀₋₃-cycloalkyl^{a}, —(CH₂)₀₋₃-heterocycloalkyl, —(CH(alkyl)-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, —(C(H)(heteroaryl))-alkyl, —(C(H)(alkyl))—(C═O)—OH, and —(C(H)(heteroaryl))—(C═O)—OR_{28;}
or R₄ and R₅ together with the nitrogen atom to which they are attached can form a carbon-containing 4-, 5-, or 6-membered saturated heterocyclic ring, optionally containing an additional heteroatom selected from N, and O, which may be optionally mono- or di-substituted with substituents selected from oxo, alkyl, alkoxy, OH, halo and CF₃; and
R₆ is H, small alkyl, and —(CH₂)-phenyl; and
R₃ can be substituted with one or two substituents independently selected from small alkyl, small alkoxy, CF₃, halo, —C(═O)—NH₂, and CN.
B₁ can be a carbon centre and B₂ can be a carbon centre, wherein B₃ is a single bond between B₂ and the nitrogen atom to which R₁ is attached and B₁ is connected to B₂ via a single bond, or B₁ can be a nitrogen centre and B₂ can be a carbon centre wherein B₁ is connected to B₂ via a double bond; B₄ can be N(R₂), B₅ can be N, and B₆ can be C(R₃), wherein B₄ is connected to B₅ via a single bond and B₅ is connected to B₆ via a double bond, or B₄ can be C(R₂), B₅ can be N, and B₆ can be N(R₃), wherein B₄ is connected to B₅ via a double bond and B₅ is connected to B₆ via a single bond; R₂ can be selected from —C(═O)—N(R₄)(R₅),

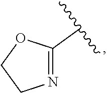 [SMILES: *C1=NCCO1]

—C(═O)—O—R₆,—CH₂—NH—C(═O) CH₃,—CH₂—NH—S(═O)₂(cyclopropyl),—CH₂—O—R₁₆,—CH₂—OH, —CN, and wherein:
R₄ is H or small alkyl, and
R₅ is selected from alkyl, OH, —S(═O)₂-(small alkyl), —(CH₂)₁₋₃—(C═O)—OH, —(CH₂)₀₋₃-cycloalkyl^{a}, —(CH₂)₀₋₃-heterocycloalkyl, —(CH(alkyl)-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, —(C(H)(heteroaryl))-alkyl, —(C(H)(alkyl))—(C═O)—OH, and —(C(H)(heteroaryl))—(C═O)—OR_{28;}
or R₄ and R₅ together with the nitrogen atom to which they are attached can form a carbon-containing 4-, 5-, or 6-membered saturated heterocyclic ring, optionally containing an additional heteroatom selected from N, and O, which may be optionally mono- or di-substituted with substituents selected from oxo, alkyl, alkoxy, OH, halo and CF₃; and R₆ is H, small alkyl, and —(CH₂)-phenyl; and
R₃ can be substituted with one or two substituents independently selected from small alkyl, small alkoxy, CF₃, halo, —C(═O)—NH₂, and CN.
R₁ can be:
(i) Formula (IIb),

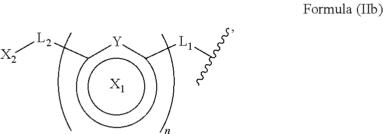

wherein:
n is 1; L₁ is a linker selected from a single bond, —(CH₂)₁₋₃—, —(CH)(alkyl)-, and —C(alkyl)(alkyl)-; Y is selected from C, N, O and S;
X₁ is phenyl or a 5- or 6-membered carbon-containing aromatic ring containing one, two or three ring members that are selected from N, S, and O;
wherein X₁ may be optionally substituted with 1 or 2 substituents independently selected from alkyl, cycloalkyl, alkoxy, OH, CF₃, and halo;
L₂ is a linker selected from a single bond, —(CH₂)₁₋₃—, —(CH)(alkyl)-, —C(alkyl)(alkyl)-, —C(═O)—, and —SO₂—; and
X₂ is either:
(a) NR₂OR₂₁, wherein R₂₀ is selected from H, alkyl, and —(CH₂)₀₋₃-R_{22;}
wherein R₂₁ is selected from H, alkyl, and —(CH₂)₀₋₃-R₂₃;

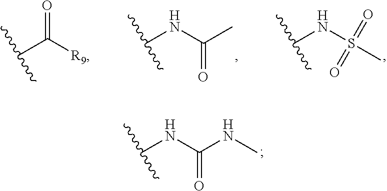

wherein R₂₂ is selected from cycloalkyl, heterocycloalkyl, aryl, heteroaryl,

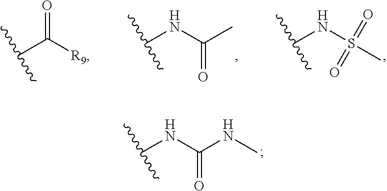

wherein R₂₃ is selected from cycloalkyl, heterocycloalkyl, aryl, heteroaryl, or
(b)₂-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected from alkyl, cycloalkyl, OH, oxo, CF₃, and halo, alkoxy, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O, and X₂ may be further optionally substituted with 1 or 2 substituents R₂₉ and R₃₀, wherein R₂₉ and R₃₀ are independently selected from alkyl, cycloalkyl, alkoxy, OH, oxo, CF₃, and halo; or

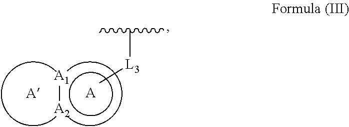

wherein:
L₃ is a single bond;
A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R is selected from alkyl, alkoxy, and —(CH₂)₀₋₃-R₈,

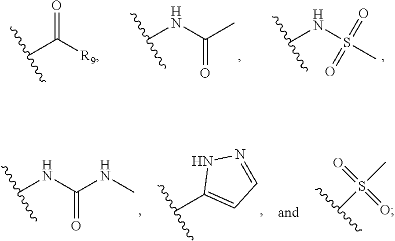

wherein R₈ is selected from cycloalkyl, heterocycloalkyl, wherein R₉ is selected from H, alkyl, OH, N(R₃₁)(R₃₂), and alkoxy;
wherein R₃₁ is H or small alkyl;
wherein R₃₂ is selected from H, small alkyl, and —(CH₂)₀₋₃heteroaryl;
wherein A′ may be optionally substituted with 1, 2, or 3 further substituents R₁₀, R₁₁, and R₁₇, wherein R₁₀, R₁₁, and R₁₇, are independently selected from alkyl, cycloalkyl, alkoxy, OH, oxo, CF₃, and halo, or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O; and
R₃ can be substituted with one or two substituents independently selected from small alkyl, small alkoxy, CF₃, halo, —C(═O)—NH₂, and CN.
B₁ can be a carbon centre and B₂ can be a carbon centre, wherein B₃ is a single bond between B₂ and the nitrogen atom to which R₁ is attached and B₁ is connected to B₂ via a single bond, or B₁ can be a nitrogen centre and B₂ can be a carbon centre wherein B₁ is connected to B₂ via a double bond;
R₁ can be:
(i) Formula (IIb),

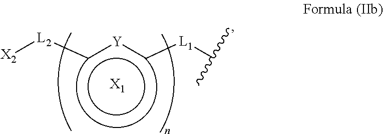

wherein:

nis 1;
L₁ is a linker selected from a single bond, —(CH₂)₁₋₃—, —(CH)(alkyl)-, and —C(alkyl)(alkyl)-; Y is selected from C, N, O and S;
X₁ is phenyl or a 5- or 6-membered carbon-containing aromatic ring containing one, two or three ring members that are selected from N, S, and O;
wherein X₁ may be optionally substituted with 1 or 2 substituents independently selected from alkyl, cycloalkyl, alkoxy, OH, CF₃, and halo;
L₂ is a linker selected from a single bond, —(CH₂)₁₋₃—, —(CH)(alkyl)-, —C(alkyl)(alkyl)-, —C(═O)—, and —SO₂—; and
X₂ is either:
(a) —NR₂OR₂₁, wherein R₂₀ is selected from H, alkyl, and —(CH₂)₀₋₃-R_{22;}
wherein R₂₁ is selected from H, alkyl, and —(CHz)₀₋₃-R₂₃;

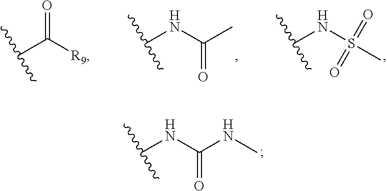

wherein R₂₂ is selected from cycloalkyl, heterocycloalkyl, aryl, heteroaryl,

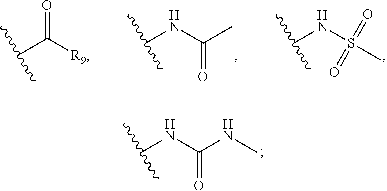

wherein R₂₃ is selected from cycloalkyl, heterocycloalkyl, aryl, heteroaryl, or
(b)₂-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O, and X₂ may be further optionally substituted with 1 or 2 substituents R₂₉ and R₃₀, wherein R₂₉ and R₃₀ are independently selected from alkyl, cycloalkyl, alkoxy, OH, oxo, CF₃, and halo; or
(ii) Formula (III) comprising an aromatic ring A fused to a non-aromatic ring A′,

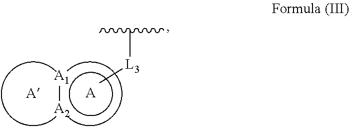

wherein:
L₃ is a single bond;
A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R, is selected from alkyl, alkoxy, and —(CH₂)₀₋₃-R₈,

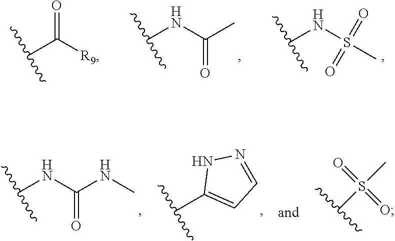

wherein R₈ is selected from cycloalkyl, heterocycloalkyl, wherein R₉ is selected from H, alkyl, OH, N(R₃₁)(R₃₂), and alkoxy;
wherein R₃₁ is H or small alkyl;
wherein R₃₂ is selected from H, small alkyl, and —(CH₂)₀₋₃heteroaryl;
wherein A′ may be optionally substituted with 1, 2, or 3 further substituents R₁₀, R₁₁, and R₁₇, wherein R₁₀, R₁₁, and R₁₇, are independently selected from alkyl, cycloalkyl, alkoxy, OH, oxo, CF₃, and halo, or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O; and
R₃ can be substituted with one or two substituents independently selected from small alkyl, small alkoxy, CF₃, halo, —C(═O)—NH₂, and CN.
B₄ can be N(R₂), B₅ can be N, and B₆ can be C(R₃), wherein B₄ is connected to B₅ via a single bond and B₅ is connected to B₆ via a double bond, or B₄ can be C(R₂), Bs can be N, and B₆ can be N(R₃), wherein B₄ is connected to B₅ via a double bond and B₅ is connected to B₆ via a single bond;
R₁ can be:
(i) Formula (IIb),

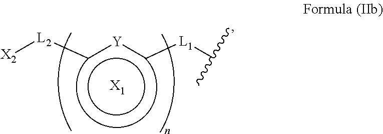

wherein:
n is 1;
L₁ is a linker selected from a single bond, —(CH₂)₁₋₃—, —(CH)(alkyl)-, and —C(alkyl)(alkyl)-; Y is selected from C, N, O and S;
X₁ is phenyl or a 5- or 6-membered carbon-containing aromatic ring containing one, two or three ring members that are selected from N, S, and O;
wherein X₁ may be optionally substituted with 1 or 2 substituents independently selected from alkyl, cycloalkyl, alkoxy, OH, CF₃, and halo;
L₂ is a linker selected from a single bond, —(CH₂)₁₋₃—, —(CH)(alkyl)-, —C(alkyl)(alkyl)-, —C(═O)—, and —SO₂—; and either:
wherein R₂₀ is selected from H, alkyl, and —(CH₂)₀₋₃-R_{22;}
wherein R₂₁ is selected from H, alkyl, and —(CH₂)₀₋₃-R_{23;}
X₂ is (a) —NR₂OR₂₁,

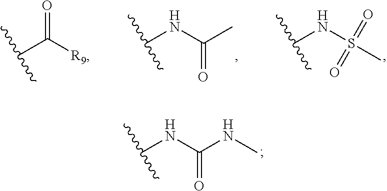

wherein R₂₂ is selected from cycloalkyl, heterocycloalkyl, aryl, heteroaryl,

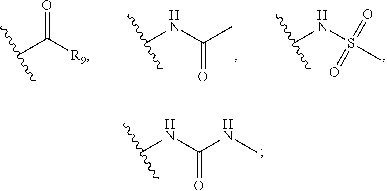

wherein R₂₃ is selected from cycloalkyl, heterocycloalkyl, aryl, heteroaryl, or
(b)₂-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O, and X₂ may be further optionally substituted with 1 or 2 substituents R₂₉ and R₃₀, wherein R₂₉ and R₃₀ are independently selected from alkyl, cycloalkyl, alkoxy, OH, oxo, CF₃, and halo; or

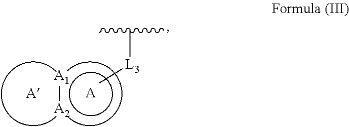

wherein:
L₃ is a single bond;
A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R₇ is selected from alkyl, alkoxy, and —(CH₂)₀₋₃-R₈,

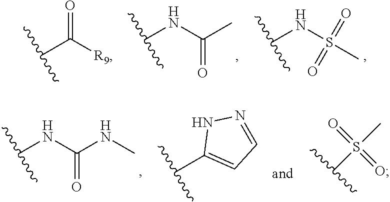

wherein R₈ is selected from cycloalkyl, heterocycloalkyl, wherein R₉ is selected from H, alkyl, OH, N(R₃₁)(R₃₂), and alkoxy;
wherein R₃₁ is H or small alkyl;
wherein R₃₂ is selected from H, small alkyl, and —(CH₂)₀₋₃heteroaryl;
wherein A′ may be optionally substituted with 1, 2, or 3 further substituents R₁₀, R₁₁, and R₁₇, wherein R₁₀, R₁₁, and R₁₇, are independently selected from alkyl, cycloalkyl, alkoxy, OH, oxo, CF₃, and halo, or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O; and
R₃ can be substituted with one or two substituents independently selected from small alkyl, small alkoxy, CF₃, halo, —C(═O)—NH₂, and CN.
B₁ can be a carbon centre and B₂ can be a carbon centre, wherein B₃ is a single bond between B₂ and the nitrogen atom to which R₁ is attached and B₁ is connected to B₂ via a single bond, or B₁ can be a nitrogen centre and B₂ can be a carbon centre wherein B₁ is connected to B₂ via a double bond; B₄ can be N(R₂), B₅ can be N, and B₆ can be C(R₃), wherein B₄ is connected to B₅ via a single bond and B₅ is connected to B₆ via a double bond, or B₄ can be C(R₂), B₅ can be N, and B₆ can be N(R₃), wherein B₄ is connected to B₅ via a double bond and B₅ is connected to B₆ via a single bond;
R₁ can be:
(i) Formula (IIb),

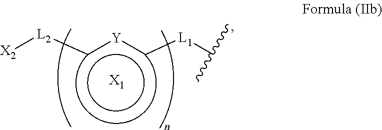

wherein:
n is 1;
L₁ is a linker selected from a single bond, —(CH₂)₁₋₃—, —(CH)(alkyl)-, and —C(alkyl)(alkyl)-; Y is selected from C, N, O and S;
X₁ is phenyl or a 5- or 6-membered carbon-containing aromatic ring containing one, two or three ring members that are selected from N, S, and O;
wherein X₁ may be optionally substituted with 1 or 2 substituents independently selected from alkyl, cycloalkyl, alkoxy, OH, CF₃, and halo;
L₂ is a linker selected from a single bond, —(CH₂)₁₋₃—, —(CH)(alkyl)-, —C(alkyl)(alkyl)-, —C(═O)—, and —SO₂—; and
X₂ is either:
(a) —NR₂OR₂₁, wherein R₂₀ is selected from H, alkyl, and —(CH₂)₀₋₃-R_{22;}
wherein R₂₁ is selected from H, alkyl, and —(CH₂)₀₋₃-R₂₃;

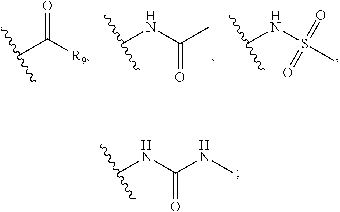

wherein R₂₂ is selected from cycloalkyl, heterocycloalkyl, aryl, heteroaryl,

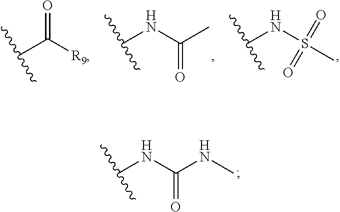

wherein R₂₃ is selected from cycloalkyl, heterocycloalkyl, aryl, heteroaryl, or
(b)₂-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O, and X₂ may be further optionally substituted with 1 or 2 substituents R₂₉ and R₃₀, wherein R₂₉ and R₃₀ are independently selected from alkyl, cycloalkyl, alkoxy, OH, oxo, CF₃, and halo; or
(ii) Formula (III) comprising an aromatic ring A fused to a non-aromatic ring A′,

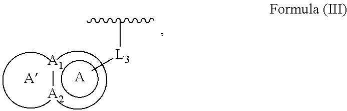

wherein:
L₃ is a single bond;
A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R₇ is selected from alkyl, alkoxy, and —(CH₂)₀₋₃-R₈,

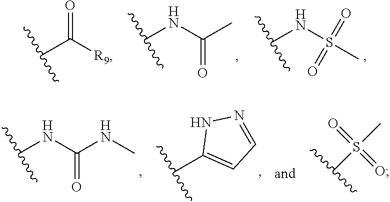

wherein R₈ is selected from cycloalkyl, heterocycloalkyl, wherein R₉ is selected from H, alkyl, OH, N(R₃₁)(R₃₂), and alkoxy;
wherein R₃₁ is H or small alkyl;
wherein R₃₂ is selected from H, small alkyl, and —(CH₂)₀₋₃heteroaryl;
wherein A′ may be optionally substituted with 1, 2, or 3 further substituents R₁₀, R₁₁, and R₁₇, wherein R₁₀, R₁₁, and R₁₇, are independently selected from alkyl, cycloalkyl, alkoxy, OH, oxo, CF₃, and halo, or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O; and R₃ can be substituted with one or two substituents independently selected from small alkyl, small alkoxy, CF₃, halo, —C(═O)—NH₂, and CN.
B₁ can be a carbon centre and B₂ can be a carbon centre, wherein B₃ is a single bond between B₂ and the nitrogen atom to which R₁ is attached and B₁ is connected to B₂ via a single bond, or B₁ can be a nitrogen centre and B₂ can be a carbon centre wherein B₁ is connected to B₂ via a double bond; R₂ can be selected from —C(═O)—N(R₄)(R₅), —C(═O)—O—R₆,—CH₂—NH—C(═O) CH₃,—CH₂—NH—S(═O)₂(cyclopropyl),—CH₂—O—R₁₆,—CH₂—OH,

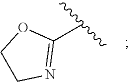 [SMILES: *C1=NCCO1]

—CN, and
R₁ can be:

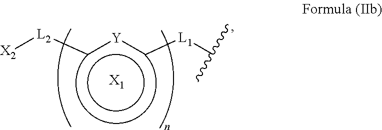

wherein:
n is 1;
L₁ is a linker selected from a single bond, —(CH₂)₁₋₃—, —(CH)(alkyl)-, and —C(alkyl)(alkyl)-; Y is selected from C, N, O and S;
X₁ is phenyl or a 5- or 6-membered carbon-containing aromatic ring containing one, two or three ring members that are selected from N, S, and O;
wherein X₁ may be optionally substituted with 1 or 2 substituents independently selected from alkyl, cycloalkyl, alkoxy, OH, CF₃, and halo;
L₂ is a linker selected from a single bond, —(CH₂)₁₋₃—, —(CH)(alkyl)-, —C(alkyl)(alkyl)-, —C(═O)—, and —SO₂—; and
X₂ is either: (a) —NR₂OR₂₁, wherein R₂₀ is selected from H, alkyl, and —(CH₂)₀₋₃-R_{22;}
wherein R₂₁ is selected from H, alkyl, and —(CH₂)₀₋₃-R₂₃; <CWU-Call number= “827” />wherein R₂₂ is selected from cycloalkyl, heterocycloalkyl, aryl, heteroaryl,

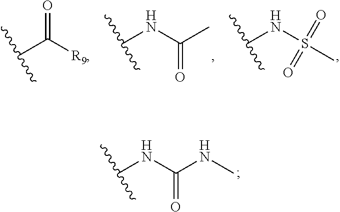

wherein R₂₃ is selected from cycloalkyl, heterocycloalkyl, aryl, heteroaryl,

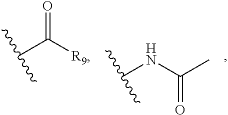

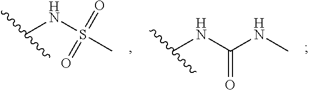

or
(b)₂-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected alkyl, cycloalkyl, OH, from alkoxy, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O, and X₂ may be further optionally substituted with 1 or 2 substituents R₂₉ and R₃₀, wherein R₂₉ and R₃₀ are independently selected from alkyl, cycloalkyl, alkoxy, OH, oxo, CF₃, and halo; or

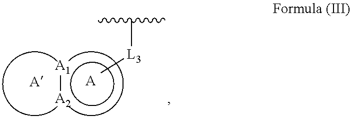

wherein:
L₃ is a single bond;
A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R₇ is selected from alkyl, alkoxy, and —(CH₂)₀₋₃-R₈,

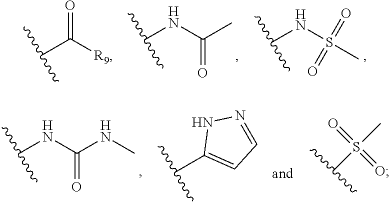

wherein R₅ is selected from cycloalkyl, heterocycloalkyl, wherein R₉ is selected from H, alkyl, OH, N(R₃₁)(R₃₂), and alkoxy;
wherein R₃₁ is H or small alkyl;
wherein R₃₂ is selected from H, small alkyl, and —(CH₂)₀₋₃heteroaryl;
wherein A′ may be optionally substituted with 1, 2, or 3 further substituents R₁₀, R₁₁, and R₁₇, wherein R₁₀, R₁₁, and R₁₇, are independently selected from alkyl, cycloalkyl, alkoxy, OH, oxo, CF₃, and halo, or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O; and
R₃ can be substituted with one or two substituents independently selected from small alkyl, small alkoxy, CF₃, halo, —C(═O)—NH₂, and CN.
B₄ can be N(R₂), B₅ can be N, and B₆ can be C(R₃), wherein B₄ is connected to B₅ via a single bond and Bs is connected to B₆ via a double bond, or B₄ can be C(R₂), B₅ can be N, and B₆ can be N(R₃), wherein B₄ is connected to B₅ via a double bond and B₅ is connected to B₆ via a single bond; R₂ can be selected from-C(═O)—N(R₄)(R₅), —C(═O)—O—R₆,—CH₂—NH—C(═O) CH₃,—CH₂—NH—S(═O)₂(cyclopropyl),—CH₂—O—R₁₆,—CH₂—OH, —CN,

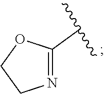 [SMILES: *C1=NCCO1]

and
R₁ can be:
(i) Formula (IIb),

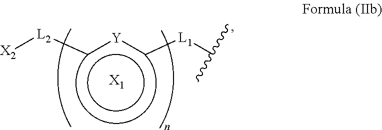

wherein:
n is 1;
L₁ is a linker selected from a single bond, —(CH₂)₁₋₃—, —(CH)(alkyl)-, and —C(alkyl)(alkyl)-;
Y is selected from C, N, O and S;
X₁ is phenyl or a 5- or 6-membered carbon-containing aromatic ring containing one, two or three ring members that are selected from N, S, and O;
wherein X₁ may be optionally substituted with 1 or 2 substituents independently selected from alkyl, cycloalkyl, alkoxy, OH, CF₃, and halo;
L₂ is a linker selected from a single bond, —(CH₂)₁₋₃—, —(CH)(alkyl)-, —C(alkyl)(alkyl)-, —C(═O)—, and —SO₂—; and
X₂ is either:
(a) NR₂OR₂₁, wherein R₂₀ is selected from H, alkyl, and —(CH₂)₀₋₃-R_{22;}
wherein R₂₁ is selected from H, alkyl, and —(CH₂)₀₋₃-R₂₃;

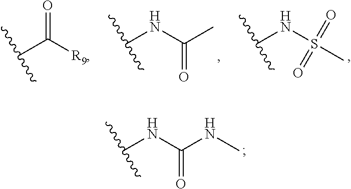

wherein R₂₂ is selected from cycloalkyl, heterocycloalkyl, aryl, heteroaryl,

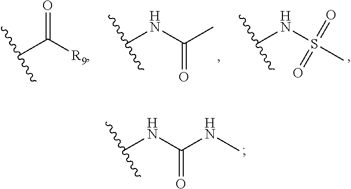

wherein R₂₃ is selected from cycloalkyl, heterocycloalkyl, aryl, heteroaryl, or
(b)₂-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O, and X₂ may be further optionally substituted with 1 or 2 substituents R₂₉ and R₃₀, wherein R₂₉ and R₃₀ are independently selected from alkyl, cycloalkyl, alkoxy, OH, oxo, CF₃, and halo; or
(ii) Formula (III) comprising an aromatic ring A fused to a non-aromatic ring A′,

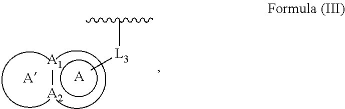

wherein:
L₃ is a single bond;
A₁ and A₂ are both carbon;
A is phenyl or a 5- or 6-membered carbon-containing aromatic ring containing one, two or three
wherein A may be optionally substituted with 1 or 2 substituents independently selected from alkyl, cycloalkyl, alkoxy, OH, CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R₇ is selected from alkyl, alkoxy, and —(CH₂)₀₋₃-R₈,

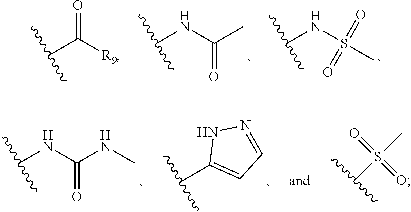

wherein R₈ is selected from cycloalkyl, heterocycloalkyl, wherein R₉ is selected from H, alkyl, OH, N(R₃₁)(R₃₂), and alkoxy;
wherein R₃₁ is H or small alkyl;
wherein R₃₂ is selected from H, small alkyl, and —(CH₂)₀₋₃heteroaryl;
wherein A′ may be optionally substituted with 1, 2, or 3 further substituents R₁₀, R₁₁, and R₁₇, wherein R₁₀, R₁₁, and R₁₇, are independently selected from alkyl, cycloalkyl, alkoxy, OH, oxo, CF₃, and halo, or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O; and
R₃ can be substituted with one or two substituents independently selected from small alkyl, small alkoxy, CF₃, halo, —C(═O)—NH₂, and CN.
B₁ can be a carbon centre and B₂ can be a carbon centre, wherein B₃ is a single bond between B₂ and the nitrogen atom to which R₁ is attached and B₁ is connected to B₂ via a single bond, or B₁ can be a nitrogen centre and B₂ can be a carbon centre wherein B₁ is connected to B₂ via a double bond; B₄ can be N(R₂), B₅ can be N, and B₆ can be C(R₃), wherein B₄ is connected to B₅ via a single bond and B₅ is connected to B₆ via a double bond, or B₄ can be C(R₂), B₅ can be N, and B₆ can be N(R₃), wherein B₄ is connected to B₅ via a double bond and B₅ is connected to B₆ via a single bond; R₂ can be selected from —C(═O)—N(R₄)(R₅), —C(═O)—

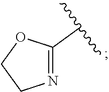 [SMILES: *C1=NCCO1]

O—R₆,—CH₂—NH—C(═O) CH₃,—CH₂—NH—S(═O)₂(cyclopropyl),—CH₂—O—R₁₆,—CH₂—OH, —CN, and

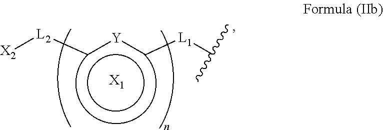

wherein:
n is 1;
L₁ is a linker selected from a single bond, —(CH₂)₁₋₃—, —(CH)(alkyl)-, and —C(alkyl)(alkyl)-; Y is selected from C, N, O and S;
X₁ is phenyl or a 5- or 6-membered carbon-containing aromatic ring containing one, two or three ring members that are selected from N, S, and O;
wherein X₁ may be optionally substituted with 1 or 2 substituents independently selected from alkyl, cycloalkyl, alkoxy, OH, CF₃, and halo; L₂ is a linker selected from a single bond, —(CH₂)₁₋₃—, —(CH)(alkyl)-, —C(alkyl)(alkyl)-, —C(═O)—, and —SO₂—; and
X₂ is either:
(a) —NR₂OR₂₁, wherein R₂₀ is selected from H, alkyl, and —(CH₂)₀₋₃-R_{22;}
wherein R₂₁ is selected from H, alkyl, and —(CH₂)₀₋₃-R₂₃;

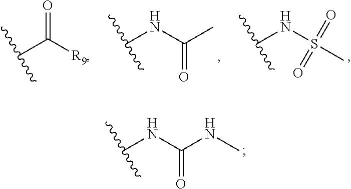

wherein R₂₂ is selected from cycloalkyl, heterocycloalkyl, aryl, heteroaryl,

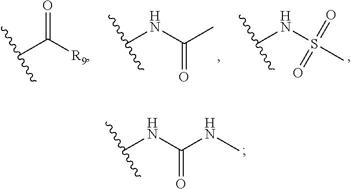

wherein R₂₃ is selected from cycloalkyl, heterocycloalkyl, aryl, heteroaryl, or
(b)₂-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected from alkyl, cycloalkyl, alkoxy, OH, Oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O, and X₂ may be further optionally substituted with 1 or 2 substituents R₂₉ and R₃₀, wherein R₂₉ and R₃₀ are independently selected from alkyl, cycloalkyl, alkoxy, OH, oxo, CF₃, and halo; or Formula (III) comprising an aromatic ring A fused to a non-aromatic ring A′,
(ii)

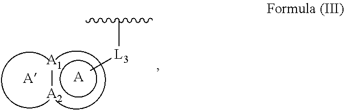

wherein:
L₃ is a single bond;
A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R₇ is selected from alkyl, alkoxy, and —(CH₂)₀₋₃-R₈,

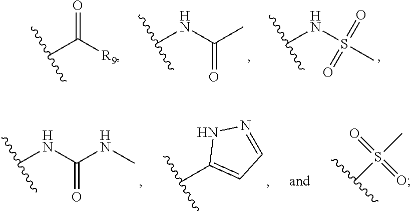

wherein R& is selected from cycloalkyl, heterocycloalkyl, wherein R₉ is selected from H, alkyl, OH, N(R₃₁)(R₃₂), and alkoxy;
wherein R₃₁ is H or small alkyl;
wherein R₃₂ is selected from H, small alkyl, and —(CH₂)₀₋₃heteroaryl;
wherein A′ may be optionally substituted with 1, 2, or 3 further substituents Rio, R₁₁, and R₁₇, wherein R₁₀, R₁₁, and R₁₇, are independently selected from alkyl, cycloalkyl, alkoxy, OH, oxo, CF₃, and halo, or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O; and
R₃ can be substituted with one or two substituents independently selected from small alkyl, small alkoxy,

CF₃, halo, —C(═O)—NH₂, and CN.
R₂ can be selected from —C(═O)—N(R₄)(R₅), —C(═O)—O—R₆,—CH₂—NH—C(═O) CH₃,—CH₂—NH—S(═O)₂(cyclopropyl),

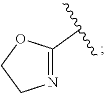 [SMILES: *C1=NCCO1]

—CH₂—O—R₁₆,—CH₂—OH, —CN, and
R₁ can be:
(i) Formula (IIb),

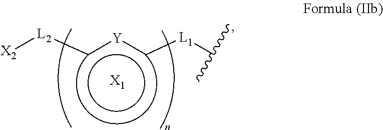

wherein:
n is 1;
L₁ is a linker selected from a single bond, —(CH₂)₁₋₃—, —(CH)(alkyl)-, and —C(alkyl)(alkyl)-; Y is selected from C, N, O and S;
X₁ is phenyl or a 5- or 6-membered carbon-containing aromatic ring containing one, two or three ring members that are selected from N, S, and O;
wherein X₁ may be optionally substituted with 1 or 2 substituents independently selected from alkyl, cycloalkyl, alkoxy, OH, CF₃, and halo;
L₂ is a linker selected from a single bond, —(CH₂)₁₋₃—, —(CH)(alkyl)-, —C(alkyl)(alkyl)-, —C(═O)—, and —SO₂—; and
X₂ is either:
(a) —NR₂OR₂₁, wherein R₂₀ is selected from H, alkyl, and —(CH₂)₀₋₃-R_{22;}
wherein R₂₁ is selected from H, alkyl, and —(CH₂)₀₋₃-R₂₃;

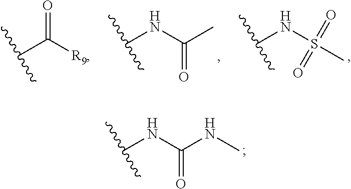

wherein R₂₂ is selected from cycloalkyl, heterocycloalkyl, aryl, heteroaryl,

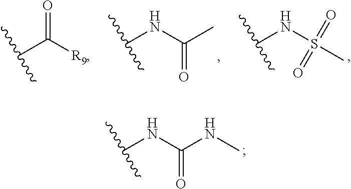

wherein R₂₃ is selected from cycloalkyl, heterocycloalkyl, aryl, heteroaryl, or
(b)₂-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected from alkyl, cycloalkyl, alkoxy, OH, Oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O, and X₂ may be further optionally substituted with 1 or 2 substituents R₂₉ and R₃₀, wherein R₂₉ and R₃₀ are independently selected from alkyl, cycloalkyl, alkoxy, OH, oxo, CF₃, and halo; or
(ii) Formula (III) comprising an aromatic ring A fused to a non-aromatic ring A′,

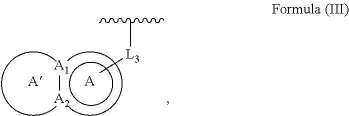

wherein:
L₃ is a single bond;
A₁ and Az are both carbon;
A is phenyl or a 5- or 6-membered carbon-containing aromatic ring containing one, two or three
wherein A may be optionally substituted with 1 or 2 substituents independently selected from alkyl, cycloalkyl, alkoxy, OH, CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R₇ is selected from alkyl, alkoxy, and —(CH, In-2-Re. wherein R₈ is selected from cycloalkyl, heterocycloalkyl,

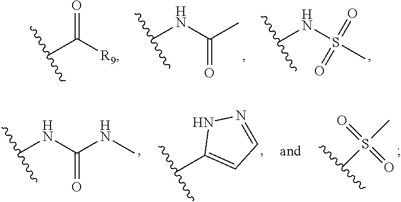

wherein R₉ is selected from H, alkyl, OH, N(R₃₁)(R₃₂), and alkoxy;
wherein R₃₁ is H or small alkyl;
wherein R₃₂ is selected from H, small alkyl, and —(CH₂)₀₋₃heteroaryl;
wherein A′ may be optionally substituted with 1, 2, or 3 further substituents R₁₀, R₁₁, and R₁₇, wherein R₁₀, R₁₁, and R₁₇, are independently selected from alkyl, cycloalkyl, alkoxy, OH, oxo, CF₃, and halo, or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O; and
R₃ can be substituted with one or two substituents independently selected from small alkyl, small alkoxy, CF₃, halo, —C(═O)—NH₂, and CN.
B₁ can be a carbon centre and B₂ can be a carbon centre, wherein B₃ is a single bond between B₂ and the nitrogen atom to which R₁ is attached and B₁ is connected to B₂ via a single bond, or B₁ can be a nitrogen centre and B₂ can be a carbon centre wherein B₁ is connected to B₂ via a double bond; R₂ can be selected from —C(═O)—N(R₄)(R₅), —C(═O)—O—R₆,—CH₂—NH—C(═O) CH₃,—CH₂—NH—S(═O)₂(cyclopropyl),—CH₂—O—R₁₆,—CH₂—OH,

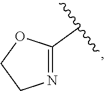 [SMILES: *C1=NCCO1]

—CN, and wherein:
R₄ is H or small alkyl, and
R₅ is selected from alkyl, OH, —S(═O)₂-(small alkyl), —(CH₂)₁₋₃—(C═O)—OH, —(CH₂)₀₋₃-cycloalkyl^{a}, —(CH₂)₀₋₃-heterocycloalkyl, —(CH(alkyl)-heterocycloalkyl, —(CH₂)₀₋₃-polycycloalkyl, —(CH₂)₀₋₃-aryl, —(C(H)(phenyl)-alkyl, —(CH₂)₀₋₃heteroaryl, —(C(H)(heteroaryl))-alkyl, —(C(H)(alkyl))—(C═O)—OH, and —(C(H)(heteroaryl))—(C═O)—OR_{28;}
or R₄ and R₅ together with the nitrogen atom to which they are attached can form a carbon-containing 4-, 5-, or 6-membered saturated heterocyclic ring, optionally containing an additional heteroatom selected from N, and O, which may be optionally mono- or di-substituted with substituents selected from oxo, alkyl, alkoxy, OH, halo and CF₃; and
R₆ is H, small alkyl, and —(CH₂)-phenyl;
R₁ can be:
(i) Formula (IIb),

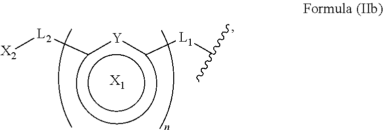

wherein:
n is 1;
L₁ is a linker selected from a single bond, —(CH₂)₁₋₃—, —(CH)(alkyl)-, and —C(alkyl)(alkyl)-;
Y is selected from C, N, O and S;
X₁ is phenyl or a 5- or 6-membered carbon-containing aromatic ring containing one, two or three ring members that are selected from N, S, and O;
wherein X₁ may be optionally substituted with 1 or 2 substituents independently selected from alkyl, cycloalkyl, alkoxy, OH, CF₃, and halo;
L₂ is a linker selected from a single bond, —(CH₂)₁₋₃—, —(CH)(alkyl)-, —C(alkyl)(alkyl)-, —C(═O)—, and —SO₂—; and
X₂ is either:
(a) —NR₂OR₂₁, wherein R₂₀ is selected from H, alkyl, and —(CH₂)₀₋₃-R_{22;}
wherein R₂₁ is selected from H, alkyl, and —(CH₂)₀₋₃-R₂₃;

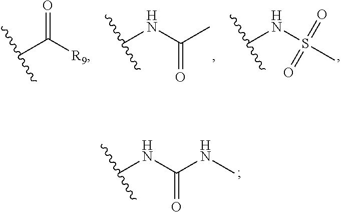

wherein R₂₂ is selected from cycloalkyl, heterocycloalkyl, aryl, heteroaryl,

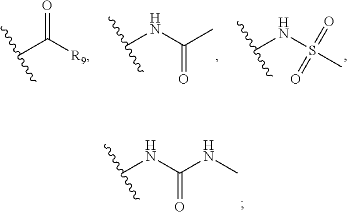

wherein R₂₃ is selected from cycloalkyl, heterocycloalkyl, aryl, heteroaryl, or
(b)₂-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O, and X₂ may be further optionally substituted with 1 or 2 substituents R₂₉ and R₃₀, wherein R₂₉ and R₃₀ are independently selected from alkyl, cycloalkyl, alkoxy, OH, oxo, CF₃, and halo; or
(ii) Formula (III) comprising an aromatic ring A fused to a non-aromatic ring A′,

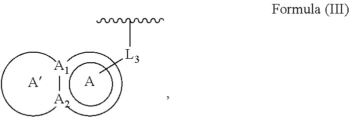

wherein:
L₃ is a single bond;
A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R₇ is selected from alkyl, alkoxy, and —(CH₂)₀₋₃-R₈,

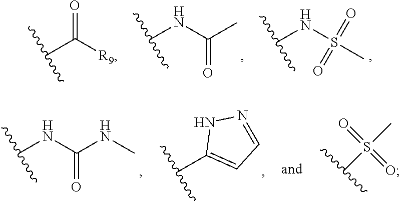

wherein R₈ is selected from cycloalkyl, heterocycloalkyl, wherein R₉ is selected from H, alkyl, OH, N(R₃₁)(R₃₂), and alkoxy;
wherein R₃₁ is H or small alkyl;
wherein R₃₂ is selected from H, small alkyl, and —(CH₂)₀₋₃heteroaryl;
wherein A′ may be optionally substituted with 1, 2, or 3 further substituents R₁₀, R₁₁, and R₁₇, wherein R₁₀, R₁₁, and R₁₇, are independently selected from alkyl, cycloalkyl, alkoxy, OH, oxo, CF₃, and halo, or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O; and
R₃ can be substituted with one or two substituents independently selected from small alkyl, small alkoxy, CF₃, halo, —C(═O)—NH₂, and CN.
B₄ can be N(R₂), B₅ can be N, and B₆ can be C(R₃), wherein B₄ is connected to B₅ via a single bond and B₅ is connected to B₆ via a double bond, or B₄ can be C(R₂), B₅ can be N, and B₆ can be N(R₃), wherein B₄ is connected to B₅ via a double bond and B₅ is connected to B₆ via a single bond; R₂ can be selected from-C(═O)—N(R₄)(R₅), —C(═O)—O—R₆,—CH₂—NH—C(═O) CH₃,—CH₂—NH—S(═O)₂(cyclopropyl),—CH₂—O—R₁₆,—CH₂—OH, —CN,

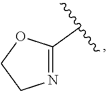 [SMILES: *C1=NCCO1]

and wherein:
R₄ is H or small alkyl, and
R₅ is selected from alkyl, OH, —S(═O)₂-(small alkyl), —(CH₂)₁₋₃—(C═O)—OH, —(CH₂)₀₋₃-cycloalkyl^{a}, —(CH₂)₀₋₃-heterocycloalkyl, —(CH(alkyl))-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, —(C(H)(heteroaryl))-alkyl, —(C(H)(alkyl))—(C═O)—OH, and —(C(H)(heteroaryl))—(C═O)—OR_{28;}
or R₄ and R₅ together with the nitrogen atom to which they are attached can form a carbon-containing 4-, 5-, or 6-membered saturated heterocyclic ring, optionally containing an additional heteroatom selected from N, and O, which may be optionally mono- or di-substituted with substituents selected from oxo, alkyl, alkoxy, OH, halo and CF₃; and R₆ is H, small alkyl, and —(CHz)-phenyl;
R₁ can be:
(i) Formula (IIb),

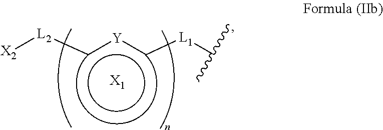

wherein:
n is 1;
L₁ is a linker selected from a single bond, —(CH₂)₁₋₃—, —(CH)(alkyl)-, and —C(alkyl)(alkyl); Y is selected from C, N, O and S;
X₁ is phenyl or a 5- or 6-membered carbon-containing aromatic ring containing one, two or three ring members that are selected from N, S, and O;
wherein X₁ may be optionally substituted with 1 or 2 substituents independently selected from alkyl, cycloalkyl, alkoxy, OH, CF₃, and halo; L₂ is a linker selected from a single bond, —(CH₂)₁₋₃—, —(CH)(alkyl)-, —C(alkyl)(alkyl)-, —C(═O)—, and —SO₂—; and
X₂ is either:
(a) —NR₂OR₂₁, wherein R₂₀ is selected from H, alkyl, and —(CH₂)₀₋₃-R_{22;}
wherein R₂₁ is selected from H, alkyl, and —(CH₂)₀₋₃-R₂₃;

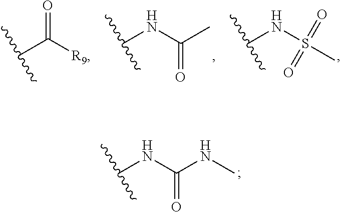

wherein R₂₂ is selected from cycloalkyl, heterocycloalkyl, aryl, heteroaryl,

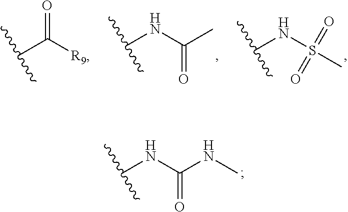

wherein Rz3 is selected from cycloalkyl, heterocycloalkyl, aryl, heteroaryl, or
(b)₂-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected from alkyl, cycloalkyl, alkoxy, CF₃, and OH, oxo, halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O, and X₂ may be further optionally substituted with 1 or 2 substituents R₂₉ and R₃₀, wherein R₂₉ and R₃₀ are independently selected from alkyl, cycloalkyl, alkoxy, OH, oxo, CF₃, and halo; or
(ii) Formula (III) comprising an aromatic ring A fused to a non-aromatic ring A′,

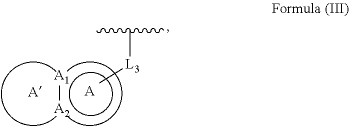

wherein:
L₃ is a single bond;
A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R₇ is selected from alkyl, alkoxy, and —(CH₂)₀₋₃-R₈,

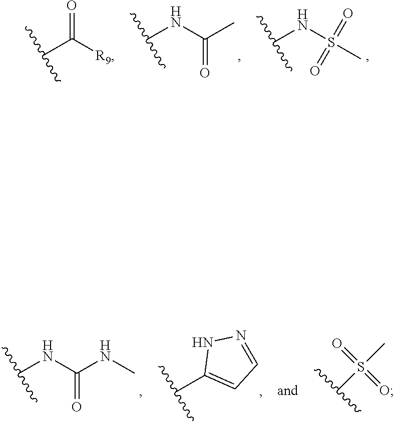

wherein R₈ is selected from cycloalkyl, heterocycloalkyl, wherein R₉ is selected from H, alkyl, OH, N(R₃₁)(R₃₂), and alkoxy;
wherein R₃₁ is H or small alkyl;
wherein R₃₂ is selected from H, small alkyl, and —(CH₂)₀₋₃heteroaryl;
wherein A′ may be optionally substituted with 1, 2, or 3 further substituents R₁₀, R₁₁, and R₁₇, wherein R₁₀, R₁₁, and R₁₇, are independently selected from alkyl, cycloalkyl, alkoxy, OH, oxo, CF₃, and halo, or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O; and
R₃ can be substituted with one or two substituents independently selected from small alkyl, small alkoxy,

CF₃, halo, —C(═O)—NH₂, and CN.
B₁ can be a carbon centre and B₂ can be a carbon centre, wherein B₃ is a single bond between B₂ and the nitrogen atom to which R₁ is attached and B₁ is connected to B₂ via a single bond, or B₁ can be a nitrogen centre and B₂ can be a carbon centre wherein B₁ is connected to B₂ via a double bond; B₄ can be N(R₂), B₅ can be N, and B₆ can be C(R₃), wherein B₄ is connected to B₅ via a single bond and B₅ is connected to B₆ via a double bond, or B₄ can be C(R₂), B₅ can be N, and B₆ can be N(R₃), wherein B₄ is connected to B₅ via a double bond and B₅ is connected to B₆ via a single bond; R₂ can be selected from —C(═O)—N(R₄)(R₅),

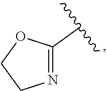 [SMILES: *C1=NCCO1]

—C(═O)—O—R₆,—CH₂—NH—C(═O) CH₃,—CH₂—NH—S(═O)₂(cyclopropyl),—CH₂—O—R₁₆,—CH₂—OH, —CN, and wherein:
R₄ is H or small alkyl, and
R₅ is selected from alkyl, OH, —S(═O)₂-(small alkyl), —(CH₂)₁₋₃—(C═O)—OH, —(CH₂)₀₋₃-cycloalkyl^{a}, —(CH₂)₀₋₃-heterocycloalkyl, —(CH(alkyl))-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, —(C(H)(heteroaryl))-alkyl, —(C(H)(alkyl))—(C═O)—OH, and —(C(H)(heteroaryl))—(C═O)—OR_{28;}
or R₄ and R₅ together with the nitrogen atom to which they are attached can form a carbon-containing 4-, 5-, or 6-membered saturated heterocyclic ring, optionally containing an additional heteroatom selected from N, and O, which may be optionally mono- or di-substituted with substituents selected from oxo, alkyl, alkoxy, OH, halo and CF₃; and R₆ is H, small alkyl, and —(CHz)-phenyl;
R₁ can be:
(i) Formula (IIb),

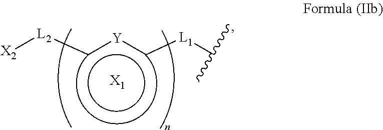

wherein:
n is 1;
L₁ is a linker selected from a single bond, —(CH₂)₁₋₃—, —(CH)(alkyl)-, and —C(alkyl)(alkyl); Y is selected from C, N, O and S;
X₁ is phenyl or a 5- or 6-membered carbon-containing aromatic ring containing one, two or three ring members that are selected from N, S, and O;
wherein X₁ may be optionally substituted with 1 or 2 substituents independently selected from alkyl, cycloalkyl, alkoxy, OH, CF₃, and halo; L₂ is a linker selected from a single bond, —(CH₂)₁₋₃—, —(CH)(alkyl)-, —C(alkyl)(alkyl)-, —C(═O)—, and —SO₂—; and
X₂ is either:
(a) —NR₂OR₂₁, wherein R₂₀ is selected from H, alkyl, and —(CH₂)₀₋₃-R_{22;}
wherein R₂₁ is selected from H, alkyl, and —(CH₂)₀₋₃-R₂₃;

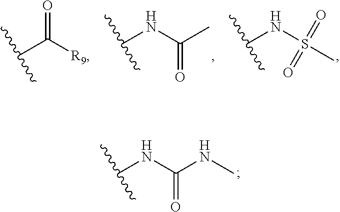

wherein R₂₂ is selected from cycloalkyl, heterocycloalkyl, aryl, heteroaryl,

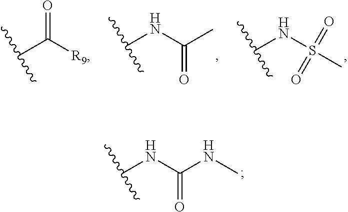

wherein R₂₃ is selected from cycloalkyl, heterocycloalkyl, aryl, heteroaryl, or
(b)₂-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O, and X₂ may be further optionally substituted with 1 or 2 substituents R₂₉ and R₃₀, wherein R₂₉ and R₃₀ are independently selected from alkyl, cycloalkyl, alkoxy, OH, oxo, CF₃, and halo; or
(ii) Formula (III) comprising an aromatic ring A fused to a non-aromatic ring A′,

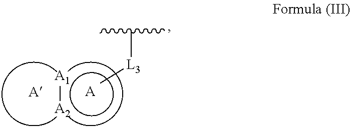

wherein:
L₃ is a single bond;
A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R₇ is selected from alkyl, alkoxy, and —(CH₂)₀₋₃-R₈,

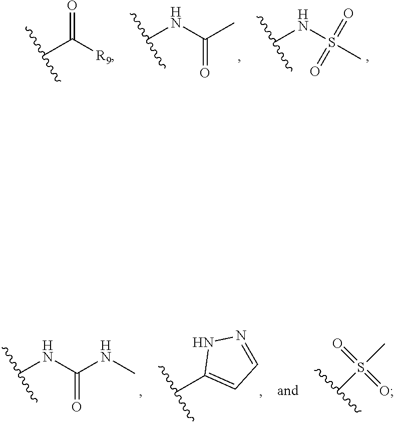

wherein R₈ is selected from cycloalkyl, heterocycloalkyl, wherein R₉ is selected from H, alkyl, OH, N(R₃₁)(R₃₂), and alkoxy;
wherein R₃₁ is H or small alkyl;
wherein R₃₂ is selected from H, small alkyl, and —(CH₂)₀₋₃heteroaryl;
wherein A′ may be optionally substituted with 1, 2, or 3 further substituents R₁₀, R₁₁, and R₁₇, wherein R₁₀, R₁₁, and R₁₇, are independently selected from alkyl, cycloalkyl, alkoxy, OH, oxo, CF₃, and halo, or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O; and
R₃ can be substituted with one or two substituents independently selected from small alkyl, small alkoxy,

CF₃, halo, —C(═O)—NH₂, and CN.
R₂ can be selected from —C(═O)—N(R₄)(R₅), —C(═O)—O—R₆,—CH₂—NH—C(═O) CH₃,—CH₂—NH—S(═O)₂(cyclopropyl), —

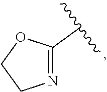 [SMILES: *C1=NCCO1]

CH₂—O—R₁₆,—CH₂—OH, —CN, and wherein:
R₄ is H or small alkyl, and
R₅ is selected from alkyl, OH, —S(═O)₂-(small alkyl), —(CH₂)₁₋₃—(C═O)—OH, —(CH₂)₀₋₃-cycloalkyl^{a}, —(CH₂)₀₋₃-heterocycloalkyl, —(CH(alkyl)-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, —(C(H)(heteroaryl))-alkyl, —(C(H)(alkyl))—(C═O)—OH, and —(C(H)(heteroaryl))—(C═O)—OR_{28;}
or R₄ and R₅ together with the nitrogen atom to which they are attached can form a carbon-containing 4-, 5-, or 6-membered saturated heterocyclic ring, optionally containing an additional heteroatom selected from N, and O, which may be optionally mono- or di-substituted with substituents selected from oxo, alkyl, alkoxy, OH, halo and CF₃; and
R₆ is H, small alkyl, and —(CH₂)-phenyl;
R₁ can be:

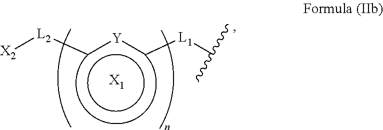

wherein:
n is 1;
L₁ is a linker selected from a single bond, —(CH₂)₁₋₃—, —(CH)(alkyl)-, and —C(alkyl)(alkyl)-; Y is selected from C, N, O and S;
X₁ is phenyl or a 5- or 6-membered carbon-containing aromatic ring containing one, two or three ring members that are selected from N, S, and O;
wherein X₁ may be optionally substituted with 1 or 2 substituents independently selected from alkyl, cycloalkyl, alkoxy, OH, CF₃, and halo; L₂ is a linker selected from a single bond, —(CH₂)₁₋₃—, —(CH)(alkyl)-, —C(alkyl)(alkyl)-, —C(═O)—, and —SO₂—; and
X₂ is either:
(a) —NR₂OR₂₁, wherein R₂₀ is selected from H, alkyl, and —(CH₂)₀₋₃-R_{22;}
wherein R₂₁ is selected from H, alkyl, and —(CH₂)₀₋₃-R₂₃;

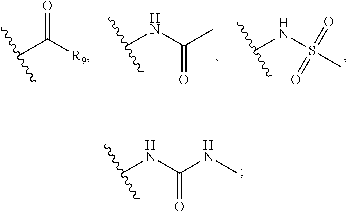

wherein R₂₂ is selected from cycloalkyl, heterocycloalkyl, aryl, heteroaryl,

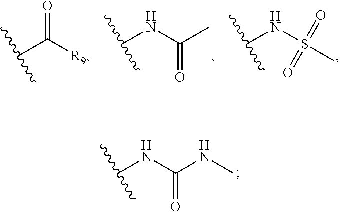

wherein R₂₃ is selected from cycloalkyl, heterocycloalkyl, aryl, heteroaryl, or
(b)₂-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected from alkyl, cycloalkyl, alkoxy, OH, Oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O, and X₂ may be further optionally substituted with 1 or 2 substituents R₂₉ and R₃₀, wherein R₂₉ and R₃₀ are independently selected from alkyl, cycloalkyl, alkoxy, OH, oxo, CF₃, and halo; or
(ii) Formula (III) comprising an aromatic ring A fused to a non-aromatic ring A′,

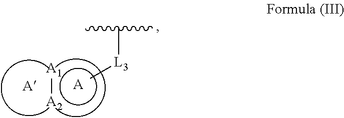

wherein:
L₃ is a single bond;
A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R₇ is selected from alkyl, alkoxy, and —(CH₂)₀₋₃-R₈,

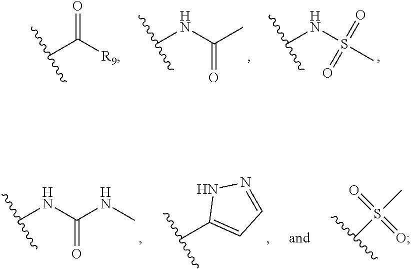

wherein R& is selected from cycloalkyl, heterocycloalkyl, wherein R₉ is selected from H, alkyl, OH, N(R₃₁)(R₃₂), and alkoxy;
wherein R₃₁ is H or small alkyl;
wherein R₃₂ is selected from H, small alkyl, and —(CH₂)₀₋₃heteroaryl;
wherein A′ may be optionally substituted with 1, 2, or 3 further substituents R₁₀, R₁₁, and R₁₇, wherein R₁₀, R₁₁, and R₁₇, are independently selected from alkyl, cycloalkyl, alkoxy, OH, oxo, CF₃, and halo, or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O; and
R₃ can be substituted with one or two substituents independently selected from small alkyl, small alkoxy,

CF₃, halo, —C(═O)—NH₂, and CN.
B₁ can be a carbon centre and B₂ can be a carbon centre, wherein B₃ is a single bond between B₂ and the nitrogen atom to which R₁ is attached and B₁ is connected to B₂ via a single bond, or B₁ can be a nitrogen centre and B₂ can be a carbon centre wherein B₁ is connected to B₂ via a double bond; and R₃ can be phenyl substituted with one or two substituents independently selected from small alkyl, small alkoxy, CF₃, halo, —C(═O)—NH₂, and CN.
B₄ can be N(R₂), B₅ can be N, and B₆ can be C(R₃), wherein B₄ is connected to B₅ via a single bond and B₅ is connected to B₆ via a double bond, or B₄ can be C(R₂), B₅ can be N, and B₆ can be N(R₃), wherein B₄ is connected to B₅ via a double bond and B₅ is connected to B₆ via a single bond; and R₃ can be phenyl substituted with one or two substituents independently selected from small alkyl, small alkoxy, CF₃, halo, —C(═O)—NH₂, and CN.
B₁ can be a carbon centre and B₂ can be a carbon centre, wherein B₃ is a single bond between B₂ and the nitrogen atom to which R₁ is attached and B₁ is connected to B₂ via a single bond, or B₁ can be a nitrogen centre and B₂ can be a carbon centre wherein B₁ is connected to B₂ via a double bond; B₄ can be N(R₂), B₅ can be N, and B₆ can be C(R₃), wherein B₄ is connected to B₅ via a single bond and B₅ is connected to B₆ via a double bond, or B₄ can be C(R₂), B₅ can be N, and B₆ can be N(R₃), wherein B₄ is connected to B₅ via a double bond and B₅ is connected to B₆ via a single bond; and R₃ can be phenyl substituted with one or two substituents independently selected from small alkyl, small alkoxy, CF₃, halo, —C(═O)—NH₂, and CN.
R₂ can be selected from —C(═O)—N(R₄)(R₅), —C(═O)—O—R₆,—CH₂—NH—S(═O)₂(cyclopropyl),—CH₂—O—CH₃,—CH₂—OH, and —CN; and R₃ can be phenyl substituted with one or two substituents independently selected from small alkyl, small alkoxy, CF₃, halo, —C(═O)—NH₂, and CN.
B₁ can be a carbon centre and B₂ can be a carbon centre, wherein B₃ is a single bond between B₂ and the nitrogen atom to which R₁ is attached and B₁ is connected to B₂ via a single bond, or B₁ can be a nitrogen centre and B₂ can be a carbon centre wherein B₁ is connected to B₂ via a double bond; R₂ can be selected from —C(═O)—N(R₄)(R₅), —C(═O)—O—R₆,—CH₂—NH—S(═O)₂(cyclopropyl),—CH₂—O—CH₃,—CH₂—OH, and —CN; and R₃ can be phenyl substituted with one or two substituents independently selected from small alkyl, small alkoxy, CF₃, halo, —C(═O)—NH₂, and CN.
B₄ can be N(R₂), B₅ can be N, and B₆ can be C(R₃), wherein B₄ is connected to Bs via a single bond and B₅ is connected to B₆ via a double bond, or B₄ can be C(R₂), B₅ can be N, and B₆ can be N(R₃), wherein B₄ is connected to B₅ via a double bond and B₅ is connected to B₆ via a single bond; R₂ can be selected from-C(═O)—N(R₄)(R₅), —C(═O)—O—R₆,—CH₂—NH—S(═O)₂(cyclopropyl),—CH₂—O—CH₃,—CH₂—OH, and —CN; and R₃ can be phenyl substituted with one or two substituents independently selected from small alkyl, small alkoxy, CF₃, halo, —C(═O)—NH₂, and CN.
B₁ can be a carbon centre and B₂ can be a carbon centre, wherein B₃ is a single bond between B₂ and the nitrogen atom to which R₁ is attached and B₁ is connected to B₂ via a single bond, or B₁ can be a nitrogen centre and B₂ can be a carbon centre wherein B₁ is connected to B₂ via a double bond; B₄ can be N(R₂), B₅ can be N, and B₆ can be C(R₃), wherein B₄ is connected to B₅ via a single bond and B₅ is connected to B₆ via a double bond, or B₄ can be C(R₂), B₅ can be N, and B₆ can be N(R₃), wherein B₄ is connected to B₅ via a double bond and B₅ is connected to B₆ via a single bond; R₂ can be selected from —C(═O)—N(R₄)(R₅), —C(═O)-O—R₆,—CH₂—NH—S(═O)₂(cyclopropyl),—CH₂—O—CH₃,—CH₂—OH, and —CN; and R₃ can be phenyl substituted with one or two substituents independently selected from small alkyl, small alkoxy, CF₃, halo, —C(═O)—NH₂, and CN.
B₁ can be a carbon centre and B₂ can be a carbon centre, wherein B₃ is a single bond between B₂ and the nitrogen atom to which R₁ is attached and B₁ is connected to B₂ via a single bond, or B₁ can be a nitrogen centre and B₂ can be a carbon centre wherein B₁ is connected to B₂ via a double bond; R₂ can be selected from —C(═O)—N(R₄)(R₅), —C(═O)—O—R₆,—CH₂—NH—S(═O)₂(cyclopropyl),—CH₂—O—CH₃,—CH₂—OH, and —CN, wherein:
R₄ is H or small alkyl, and
R₅ is selected from alkyl, —(CH₂)₀₋₃-cycloalkyl, —(CH₂)₀₋₃-heterocycloalkyl, —(CH(alkyl))-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, and —(C(H)(heteroaryl))-alkyl;
or R₄ and R₅ together with the nitrogen atom to which they are attached can form a carbon-containing 5-, or 6-membered saturated heterocyclic ring, optionally containing an additional heteroatom selected from N, and O, which may be optionally mono- or di-substituted with substituents selected from oxo, alkyl, alkoxy, OH, halo and CF₃; and
R₆ is H, small alkyl, and —(CH₂)-phenyl; and
R₃ can be phenyl substituted with one or two substituents independently selected from small alkyl, small alkoxy, CF₃, halo, —C(═O)—NH₂, and CN.
B₄ can be N(R₂), B₅ can be N, and B₆ can be C(R₃), wherein B₄ is connected to B₅ via a single bond and B₅ is connected to B₆ via a double bond, or B₄ can be C(R₂), B₅ can be N, and B₆ can be N(R₃), wherein B₄ is connected to B₅ via a double bond and B₅ is connected to B₆ via a single bond; R₂ can be selected from —C(═O)—N(R₄)(R₅), —C(═O)—O—R₆, —CH₂—NH—S(═O)₂(cyclopropyl),—CH₂—O—CH₃,—CH₂—OH, and —CN, wherein:
R₄ is H or small alkyl, and
R₅ is selected from alkyl, —(CH₂)₀₋₃-cycloalkyl, —(CH₂)₀₋₃-heterocycloalkyl, —(CH(alkyl))-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, and —(C(H)(heteroaryl))-alkyl;
or R₄ and R₅ together with the nitrogen atom to which they are attached can form a carbon-containing 5-, or 6-membered saturated heterocyclic ring, optionally containing an additional heteroatom selected from N, and O, which may be optionally mono- or di-substituted with substituents selected from oxo, alkyl, alkoxy, OH, halo and CF₃; and
R₆ is H, small alkyl, and —(CH₂)-phenyl; and
R₃ can be phenyl substituted with one or two substituents independently selected from small alkyl, small alkoxy, CF₃, halo, —C(═O)—NH₂, and CN.
B₁ can be a carbon centre and B₂ can be a carbon centre, wherein B₃ is a single bond between B₂ and the nitrogen atom to which R₁ is attached and B₁ is connected to B₂ via a single bond, or B₁ can be a nitrogen centre and B₂ can be a carbon centre wherein B₁ is connected to B₂ via a double bond; B₄ can be N(R₂), B₅ can be N, and B₆ can be C(R₃), wherein B₄ is connected to B₅ via a single bond and B₅ is connected to B₆ via a double bond, or B₄ can be C(R₂), B₅ can be N, and B₆ can be N(R₃), wherein B₄ is connected to B₅ via a double bond and B₅ is connected to B₆ via a single bond; R₂ can be selected from —C(═O)—N(R₄)(R₅), —C(═O)-O—R₆,—CH₂—NH—S(═O)₂(cyclopropyl),—CH₂—O—CH₃,—CH₂—OH, and —CN, wherein:
R₄ is H or small alkyl, and
R₅ is selected from alkyl, —(CH₂)₀₋₃-cycloalkyl, —(CH₂)₀₋₃-heterocycloalkyl, —(CH(alkyl)-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, and —(C(H)(heteroaryl))-alkyl;
or R₄ and R₅ together with the nitrogen atom to which they are attached can form a carbon-containing 5-, or 6-membered saturated heterocyclic ring, optionally containing an additional heteroatom selected from N, and O, which may be optionally mono- or di-substituted with substituents selected from oxo, alkyl, alkoxy, OH, halo and CF₃; and
R₆ is H, small alkyl, and —(CH₂)-phenyl; and
R₃ can be phenyl substituted with one or two substituents independently selected from small alkyl, small alkoxy, CF₃, halo, —C(═O)—NH₂, and CN.
R₁ can be:
(i) Formula (IIb),

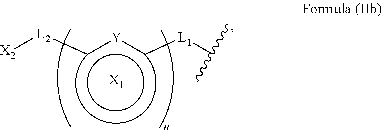

wherein:
n is 1;
L₁ is a linker selected from a single bond, —(CH₂)₁₋₃—, —(CH)(alkyl)-, and —C(alkyl)(alkyl); Y is selected from C, N, O and S;
X₁ is phenyl or a 5- or 6-membered carbon-containing aromatic ring containing one, two or three ring members that are selected from N, S, and O;
wherein X₁ may be optionally substituted with 1 or 2 substituents independently selected from alkyl, cycloalkyl, alkoxy, OH, CF₃, and halo;
L₂ is a linker selected from a single bond, —(CH₂)₁₋₃—, —(CH)(alkyl)-, —C(alkyl)(alkyl)-, —C(═O)—, and —SO₂—; and
X₂ is either:
(a) —NR₂OR₂₁, wherein R₂₀ is selected from H, alkyl, and —(CH₂)₀₋₃-R_{22;}
wherein R₂₁ is selected from H, alkyl, and —(CH₂)₀₋₃-R₂₃;

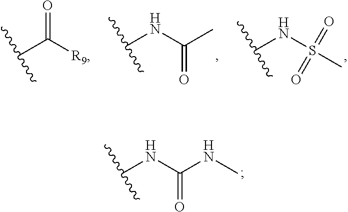

wherein R₂₂ is selected from cycloalkyl, heterocycloalkyl, aryl, heteroaryl,

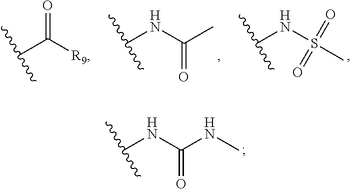

wherein R₂₃ is selected from cycloalkyl, heterocycloalkyl, aryl, heteroaryl, or
(b)₂-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected from alkyl, cycloalkyl, alkoxy, OH, Oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O; or
(ii) Formula (III) comprising an aromatic ring A fused to a non-aromatic ring A′,

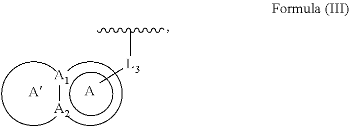

wherein:
L₃ is a single bond;
A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R₇ is selected from alkyl, alkoxy, and —(CH₂)₀₋₃-R₈,

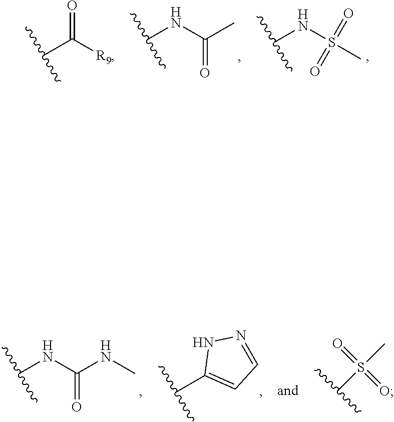

wherein R₈ is selected from cycloalkyl, heterocycloalkyl, wherein R₉ is selected from H, alkyl, OH, NH₂, and alkoxy;
wherein A′ may be optionally substituted with 1, 2, or 3 further substituents R₁₀, R₁₁, and R₁₇, wherein R₁₀, R₁₁, and R₁₇, are independently selected from alkyl, cycloalkyl, alkoxy, OH, oxo, CF₃, and halo, or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O; and
R₃ can be phenyl substituted with one or two substituents independently selected from small alkyl, small alkoxy, CF₃, halo, —C(═O)—NH₂, and CN.
B₁ can be a carbon centre and B₂ can be a carbon centre, wherein B₃ is a single bond between B₂ and the nitrogen atom to which R₁ is attached and B₁ is connected to B₂ via a single bond, or B₁ can be a nitrogen centre and B₂ can be a carbon centre wherein B₁ is connected to B₂ via a double bond;
R₁ can be:
(i) Formula (IIb),

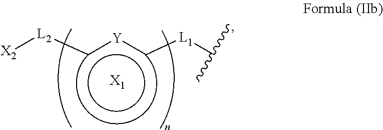

wherein:
n is 1;
L₁ is a linker selected from a single bond, —(CH₂)₁₋₃—, —(CH)(alkyl)-, and —C(alkyl)(alkyl)-;
Y is selected from C, N, O and S;
X₁ is phenyl or a 5- or 6-membered carbon-containing aromatic ring containing one, two or three ring members that are selected from N, S, and O;
wherein X₁ may be optionally substituted with 1 or 2 substituents independently selected from alkyl, cycloalkyl, alkoxy, OH, CF₃, and halo;
L₂ is a linker selected from a single bond, —(CH₂)₁₋₃—, —(CH)(alkyl)-, —C(alkyl)(alkyl)-, —C(═O)—, and —SO₂—; and
X₂ is either:
(a) —NR₂OR₂₁, wherein R₂₀ is selected from H, alkyl, and —(CH₂)₀₋₃-R_{22;}
wherein R₂₁ is selected from H, alkyl, and —(CH₂)₀₋₃-R₂₃; wherein R₂₂ is selected from cycloalkyl, heterocycloalkyl, aryl, heteroaryl,

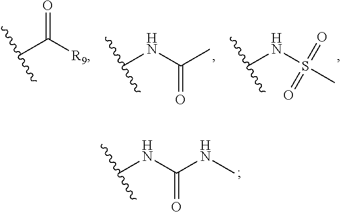

wherein R₂₃ is selected from cycloalkyl, heterocycloalkyl, aryl, heteroaryl,

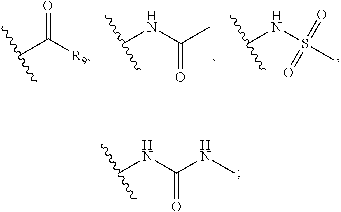

or
(b)₂-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected from alkyl, cycloalkyl, alkoxy, OH, Oxo, CF 3, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O; or
(ii) Formula (III) comprising an aromatic ring A fused to a non-aromatic ring A′,

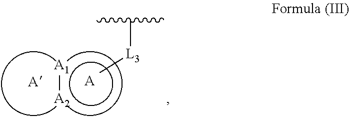

wherein:
L₃ is a single bond;
A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R₇ is selected from alkyl, alkoxy, and —(CH₂)₀₋₃-R₈,

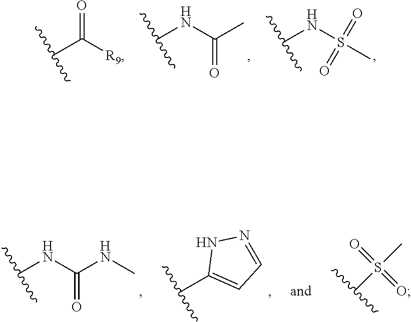

wherein R₈ is selected from cycloalkyl, heterocycloalkyl, wherein R₉ is selected from H, alkyl, OH, NH₂, and alkoxy;
wherein A′ may be optionally substituted with 1, 2, or 3 further substituents R₁₀, R₁₁, and R₁₇, wherein R₁₀, R₁₁, and R₁₇, are independently selected from alkyl, cycloalkyl, alkoxy, OH, oxo, CF₃, and halo, or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O; and
R₃ can be phenyl substituted with one or two substituents independently selected from small alkyl, small alkoxy, CF₃, halo, —C(═O)—NH₂, and CN.
B₄ can be N(R₂), B₅ can be N, and B₆ can be C(R₃), wherein B₄ is connected to B₅ via a single bond and B₅ is connected to B₆ via a double bond, or B₄ can be C(R₂), B₅ can be N, and B₆ can be N(R₃), wherein B₄ is connected to B₅ via a double bond and B₅ is connected to B₆ via a single bond;

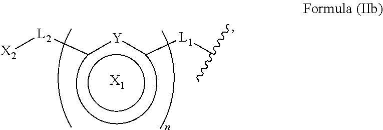

wherein:
n is 1;
L₁ is a linker selected from a single bond, —(CH₂)₁₋₃—, —(CH)(alkyl)-, and —C(alkyl)(alkyl)-; Y is selected from C, N, O and S;
X₁ is phenyl or a 5- or 6-membered carbon-containing aromatic ring containing one, two or three ring members that are selected from N, S, and O;
wherein X₁ may be optionally substituted with 1 or 2 substituents independently selected from alkyl, cycloalkyl, alkoxy, OH, CF₃, and halo; L₂ is a linker selected from a single bond, —(CH₂)₁₋₃—, —(CH)(alkyl)-, —C(alkyl)(alkyl)-, —C(═O)—, and —SO₂—; and
X₂ is either:
(a) —NR₂OR₂₁, wherein R₂₀ is selected from H, alkyl, and —(CH₂)₀₋₃-R_{22;}
wherein R₂₁ is selected from H, alkyl, and —(CH₂)₀₋₃-R₂₃;

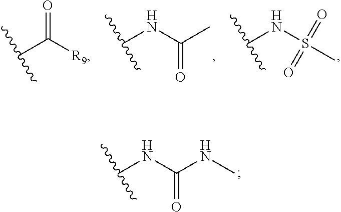

wherein R₂₂ is selected from cycloalkyl, heterocycloalkyl, aryl, heteroaryl,

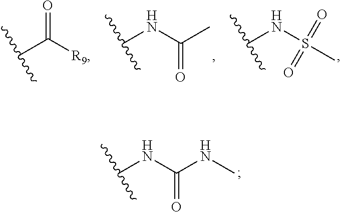

wherein R₂₃ is selected from cycloalkyl, heterocycloalkyl, aryl, heteroaryl, or
(b)₂-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected from alkyl, cycloalkyl, alkoxy, OH, Oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O; or
(ii) Formula (III) comprising an aromatic ring A fused to a non-aromatic ring A′,

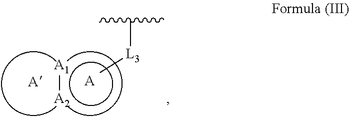

wherein:
L₃ is a single bond;
A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R₇ is selected from alkyl, alkoxy, and —(CH₂)₀₋₃-R₈,

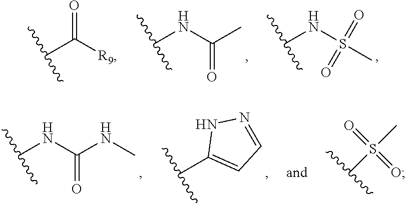

wherein R₈ is selected from cycloalkyl, heterocycloalkyl, wherein R₉ is selected from H, alkyl, OH, NH₂, and alkoxy;
wherein A′ may be optionally substituted with 1, 2, or 3 further substituents R₁₀, R₁₁, and R₁₇, wherein R₁₀, R₁₁, and R₁₇, are independently selected from alkyl, cycloalkyl, alkoxy, OH, oxo, CF₃, and halo, or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O; and
R₃ can be phenyl substituted with one or two substituents independently selected from small alkyl, small alkoxy, CF₃, halo, —C(═O)—NH₂, and CN.
B₁ can be a carbon centre and B₂ can be a carbon centre, wherein B₃ is a single bond between B₂ and the nitrogen atom to which R₁ is attached and B₁ is connected to B₂ via a single bond, or B₁ can be a nitrogen centre and B₂ can be a carbon centre wherein B₁ is connected to B₂ via a double bond; and B₄ can be N(R₂), B₅ can be N, and B₆ can be C(R₃), wherein B₄ is connected to B₅ via a single bond and B₅ is connected to B₆ via a double bond, or B₄ can be C(R₂), B₅ can be N, and B₆ can be N(R₃), wherein B₄ is connected to B₅ via a double bond and B₅ is connected to B₆ via a single bond;
R₁ can be:
(i) Formula (IIb),

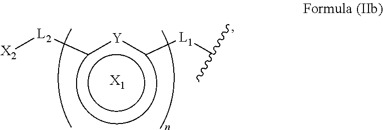

wherein:
n is 1;
L₁ is a linker selected from a single bond, —(CH₂)₁₋₃—, —(CH)(alkyl)-, and —C(alkyl)(alkyl); Y is selected from C, N, O and S;
X₁ is phenyl or a 5- or 6-membered carbon-containing aromatic ring containing one, two or three ring members that are selected from N, S, and O;
wherein X₁ may be optionally substituted with 1 or 2 substituents independently selected from alkyl, cycloalkyl, alkoxy, OH, CF₃, and halo;
L₂ is a linker selected from a single bond, —(CH₂)₁₋₃—, —(CH)(alkyl)-, —C(alkyl)(alkyl)-, —C(═O)—, and —SO₂—; and
X₂ is either:
(a) —NR₂OR₂₁, wherein R₂₀ is selected from H, alkyl, and —(CH₂)₀₋₃-R_{22;}
wherein R₂₁ is selected from H, alkyl, and —(CH₂)₀₋₃-R₂₃;

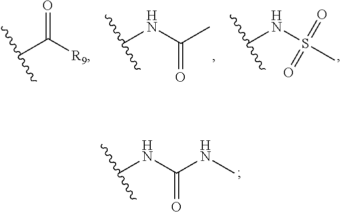

wherein R₂₂ is selected from cycloalkyl, heterocycloalkyl, aryl, heteroaryl,

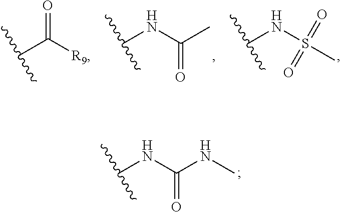

wherein R₂₃ is selected from cycloalkyl, heterocycloalkyl, aryl, heteroaryl, or
(b)₂-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected from alkyl, cycloalkyl, alkoxy, OH, Oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O; or
(ii) Formula (III) comprising an aromatic ring A fused to a non-aromatic ring A′,

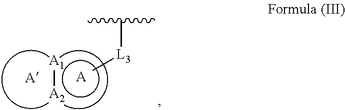

wherein:
L₃ is a single bond;
A₁ and Az are both carbon; A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R₇ is selected from alkyl, alkoxy, and —(CH₂)₀₋₃-R₈,

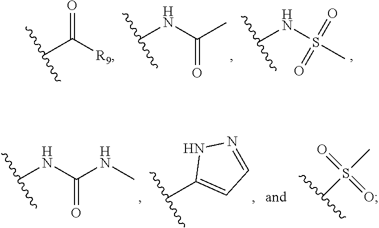

wherein R₈ is selected from cycloalkyl, heterocycloalkyl, wherein R₉ is selected from H, alkyl, OH, NH₂, and alkoxy;
wherein A′ may be optionally substituted with 1, 2, or 3 further substituents R₁₀, R₁₁, and R₁₇, wherein R₁₀, R₁₁, and R₁₇, are independently selected from alkyl, cycloalkyl, alkoxy, OH, oxo, CF₃, and halo, or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O; and R₃ can be phenyl substituted with one or two substituents independently selected from small alkyl, small alkoxy, CF₃, halo, —C(═O)—NH₂, and CN.
B₁ can be a carbon centre and B₂ can be a carbon centre, wherein B₃ is a single bond between B₂ and the nitrogen atom to which R₁ is attached and B₁ is connected to B₂ via a single bond, or B₁ can be a nitrogen centre and B₂ can be a carbon centre wherein B₁ is connected to B₂ via a double bond; R₂ can be selected from —C(═O)—N(R₄)(R₅), —C(═O)—O—R₆,—CH₂—NH—S(═O)₂(cyclopropyl),—CH₂—O—CH₃,—CH₂—OH, and —CN; and R₁ can be:
(i) Formula (IIb),

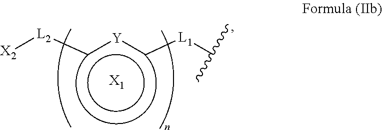

wherein:
n is 1;
L₁ is a linker selected from a single bond, —(CH₂)₁₋₃—, —(CH)(alkyl)-, and —C(alkyl)(alkyl)-;
Y is selected from C, N, O and S;
X₁ is phenyl or a 5- or 6-membered carbon-containing aromatic ring containing one, two or three ring members that are selected from N, S, and O;
wherein X₁ may be optionally substituted with 1 or 2 substituents independently selected from alkyl, cycloalkyl, alkoxy, OH, CF₃, and halo;
L₂ is a linker selected from a single bond, —(CH₂)₁₋₃—, —(CH)(alkyl)-, —C(alkyl)(alkyl)-, —C(═O)—, and —SO₂—; and
X₂ is either:
(a) —NR₂OR₂₁, wherein R₂₀ is selected from H, alkyl, and —(CH₂)₀₋₃-R_{22;}
wherein R₂₁ is selected from H, alkyl, and —(CH₂)₀₋₃-R₂₃;

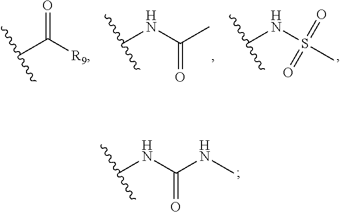

wherein R₂₂ is selected from cycloalkyl, heterocycloalkyl, aryl, heteroaryl,

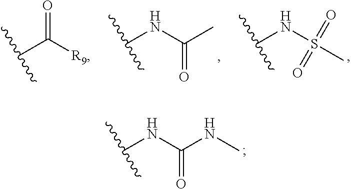

wherein R₂₃ is selected from cycloalkyl, heterocycloalkyl, aryl, heteroaryl, or
(b)₂-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected from alkyl, cycloalkyl, OH, alkoxy, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O; or

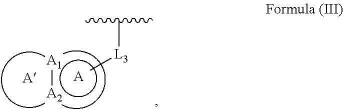

wherein:
L₃ is a single bond;
A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R₇ is selected from alkyl, alkoxy, and —(CH₂)₀₋₃-R₈,

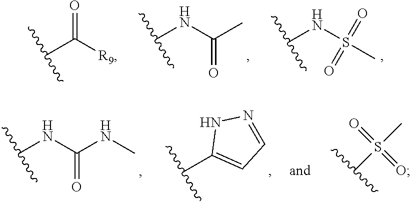

wherein R₈ is selected from cycloalkyl, heterocycloalkyl, wherein R₉ is selected from H, alkyl, OH, NH₂, and alkoxy;
wherein A′ may be optionally substituted with 1, 2, or 3 further substituents R₁₀, R₁₁, and R₁₇, wherein R₁₀, R₁₁, and R₁₇, are independently selected from alkyl, cycloalkyl, alkoxy, OH, oxo, CF₃, and halo, or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O; and
R₃ can be phenyl substituted with one or two substituents independently selected from small alkyl, small alkoxy, CF₃, halo, —C(═O)—NH₂, and CN.
B₄ can be N(R₂), B₅ can be N, and B₆ can be C(R₃), wherein B₄ is connected to B₅ via a single bond and B₅ is connected to B₆ via a double bond, or B₄ can be C(R₂), B₅ can be N, and B₆ can be N(R₃), wherein B₄ is connected to B₅ via a double bond and B₅ is connected to B₆ via a single bond; R₂ can be selected from —C(═O)—N(R₄)(R₅), —C(═O)—O—R₆,—CH₂—NH—S(═O)₂(cyclopropyl),—CH₂—O—CH₃,—CH₂—OH, and —CN; and
R₁ can be:

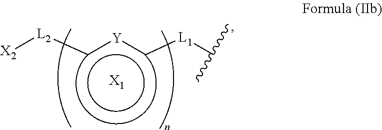

wherein:
n is 1;
L₁ is a linker selected from a single bond, —(CH₂)₁₋₃—, —(CH)(alkyl)-, and —C(alkyl)(alkyl); Y is selected from C, N, O and S;
X₁ is phenyl or a 5- or 6-membered carbon-containing aromatic ring containing one, two or three ring members that are selected from N, S, and O;
wherein X₁ may be optionally substituted with 1 or 2 substituents independently selected from alkyl, cycloalkyl, alkoxy, OH, CF₃, and halo; L₂ is a linker selected from a single bond, —(CH₂)₁₋₃—, —(CH)(alkyl)-, —C(alkyl)(alkyl)-, —C(═O)—, and —SO₂—; and
X₂ is either: (a) —NR₂OR₂₁, wherein R₂₀ is selected from H, alkyl, and —(CH₂)₀₋₃-R_{22;}
wherein R₂₁ is selected from H, alkyl, and —(CH₂)₀₋₃-R₂₃;

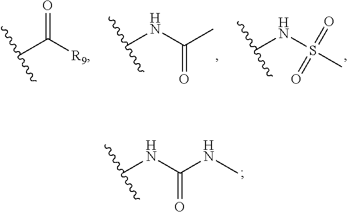

wherein R₂₂ is selected from cycloalkyl, heterocycloalkyl, aryl, heteroaryl,

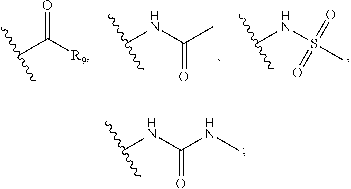

wherein R₂₃ is selected from cycloalkyl, heterocycloalkyl, aryl, heteroaryl, or
(b)₂-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O; or
(ii) Formula (III) comprising an aromatic ring A fused to a non-aromatic ring A′,

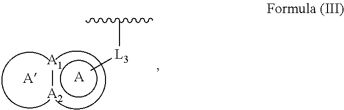

wherein:
L₃ is a single bond;
A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R₇ is selected from alkyl, alkoxy, and —(CH₂)₀₋₃-R₈,

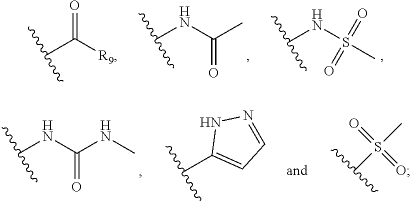

wherein R₅ is selected from cycloalkyl, heterocycloalkyl, wherein R₉ is selected from H, alkyl, OH, NH₂, and alkoxy;
wherein A′ may be optionally substituted with 1, 2, or 3 further substituents R₁₀, R₁₁, and R₁₇, wherein R₁₀, R₁₁, and R₁₇, are independently selected from alkyl, cycloalkyl, alkoxy, OH, oxo, CF₃, and halo, or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O; and R₃ can be phenyl substituted with one or two substituents independently selected from small alkyl, small alkoxy, CF₃, halo, —C(═O)—NH₂, and CN.
B₁ can be a carbon centre and B₂ can be a carbon centre, wherein B₃ is a single bond between B₂ and the nitrogen atom to which R₁ is attached and B₁ is connected to B₂ via a single bond, or B₁ can be a nitrogen centre and B₂ can be a carbon centre wherein B₁ is connected to B₂ via a double bond; B₄ can be N(R₂), B₅ can be N, and B₆ can be C(R₃), wherein B₄ is connected to B₅ via a single bond and B₅ is connected to B₆ via a double bond, or B₄ can be C(R₂), B₅ can be N, and B₆ can be N(R₃), wherein B₄ is connected to B₅ via a double bond and B₅ is connected to B₆ via a single bond; R₂ can be selected from —C(═O)—N(R₄)(R₅), —CH₂—NH—S(═O)₂(cyclopropyl), —C(═O)—O—R₆,—CH₂—O—CH₃,—CH₂—OH, and —CN; and
R₁ can be:
(i) Formula (IIb),

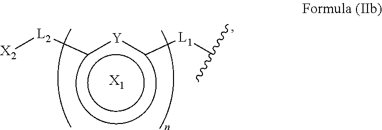

wherein:
n is 1;
L₁ is a linker selected from a single bond, —(CH₂)₁₋₃—, —(CH)(alkyl)-, and —C(alkyl)(alkyl)-; Y is selected from C, N, O and S;
X₁ is phenyl or a 5- or 6-membered carbon-containing aromatic ring containing one, two or three ring members that are selected from N, S, and O;
wherein X₁ may be optionally substituted with 1 or 2 substituents independently selected from alkyl, cycloalkyl, alkoxy, OH, CF₃, and halo;
L₂ is a linker selected from a single bond, —(CH₂)₁₋₃—, —(CH)(alkyl)-, —C(alkyl)(alkyl)-, —C(═O)—, and —SO₂—; and X₂ is either:
(a) —NR₂OR₂₁, wherein R₂₀ is selected from H, alkyl, and —(CH₂)₀₋₃-R_{22;}
wherein R₂₁ is selected from H, alkyl, and —(CH₂)₀₋₃-R₂₃;

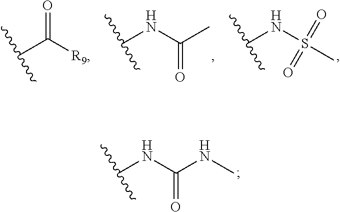

wherein R₂₂ is selected from cycloalkyl, heterocycloalkyl, aryl, heteroaryl,

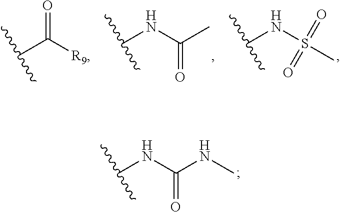

wherein R₂₃ is selected from cycloalkyl, heterocycloalkyl, aryl, heteroaryl, or
(b)₂-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O; or
(ii) Formula (III) comprising an aromatic ring A fused to a non-aromatic ring A′,

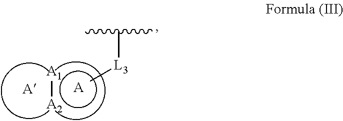

wherein:
L₃ is a single bond;
A₁ and A₂ are both carbon;
A is phenyl or a 5- or 6-membered carbon-containing aromatic ring containing one, two or three
wherein A may be optionally substituted with 1 or 2 substituents independently selected from alkyl, cycloalkyl, alkoxy, OH, CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R₇ is selected from alkyl, alkoxy, and —(CH₂)₀₋₃-R₈,

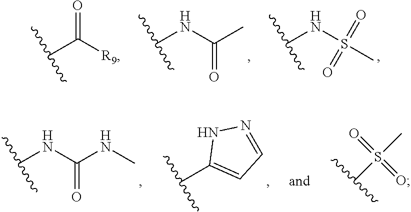

wherein R₈ is selected from cycloalkyl, heterocycloalkyl, wherein R₉ is selected from H, alkyl, OH, NH₂, and alkoxy;
wherein A′ may be optionally substituted with 1, 2, or 3 further substituents R₁₀, R₁₁, and R₁₇, wherein R₁₀, R₁₁, and R₁₇, are independently selected from alkyl, cycloalkyl, alkoxy, OH, oxo, CF₃, and halo, or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O; and
R₃ can be phenyl substituted with one or two substituents independently selected from small alkyl, small alkoxy, CF₃, halo, —C(═O)—NH₂, and CN.
R₂ can be selected from —C(═O)—N(R₄)(R₅),—CH₂—NH—S(═O)₂(cyclopropyl), —C(═O)—O—R₆,—CH₂—O—CH₃,—CH₂—OH, and —CN; and
R₁ can be:
(i) Formula (IIb),

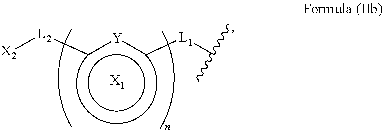

wherein:
n is 1;
L₁ is a linker selected from a single bond, —(CH₂)₁₋₃—, —(CH)(alkyl)-, and —C(alkyl)(alkyl)-;
Y is selected from C, N, O and S;
X₁ is phenyl or a 5- or 6-membered carbon-containing aromatic ring containing one, two or three ring members that are selected from N, S, and O;
wherein X₁ may be optionally substituted with 1 or 2 substituents independently selected from alkyl, cycloalkyl, alkoxy, OH, CF₃, and halo;
L₂ is a linker selected from a single bond, —(CH₂)₁₋₃—, —(CH)(alkyl)-, —C(alkyl)(alkyl)-, —C(═O)—, and —SO₂—; and
X₂ is either:
(a) —NR₂OR₂₁, wherein R₂₀ is selected from H, alkyl, and —(CH₂)₀₋₃-R_{22;}
wherein R₂₁ is selected from H, alkyl, and —(CH₂)₀₋₃-R₂₃;

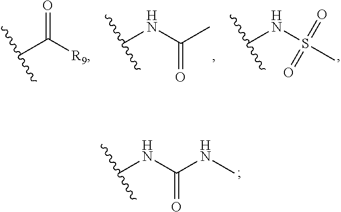

wherein R₂₂ is selected from cycloalkyl, heterocycloalkyl, aryl, heteroaryl,

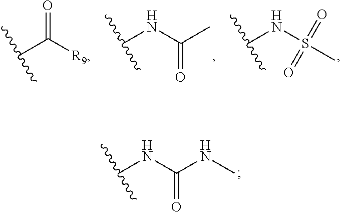

wherein R₂₃ is selected from cycloalkyl, heterocycloalkyl, aryl, heteroaryl, or
(b)₂-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected from alkyl, cycloalkyl, OH, alkoxy, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O; or

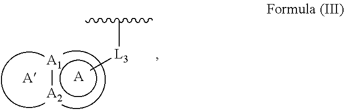

wherein:
L₃ is a single bond;
A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R₇ is selected from alkyl, alkoxy, and —(CH₂)₀₋₃-R₈,

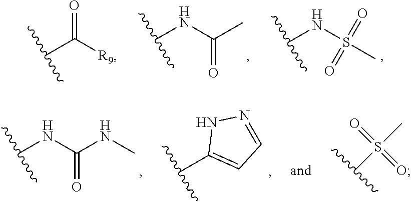

wherein R₈ is selected from cycloalkyl, heterocycloalkyl, wherein R₉ is selected from H, alkyl, OH, NH₂, and alkoxy;
wherein A′ may be optionally substituted with 1, 2, or 3 further substituents R₁₀, R₁₁, and R₁₇, wherein R₁₀, R₁₁, and R₁₇, are independently selected from alkyl, cycloalkyl, alkoxy, OH, oxo, CF₃, and halo, or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O; and
R₃ can be phenyl substituted with one or two substituents independently selected from small alkyl, small alkoxy, CF₃, halo, —C(═O)—NH₂, and CN.
B₁ can be a carbon centre and B₂ can be a carbon centre, wherein B₃ is a single bond between B₂ and the nitrogen atom to which R₁ is attached and B₁ is connected to B₂ via a single bond, or B₁ can be a nitrogen centre and B₂ can be a carbon centre wherein B₁ is connected to B₂ via a double bond;
R₂ can be selected from —C(═O)—N(R₄)(R₅),—CH₂—NH—S(═O)₂(cyclopropyl), —C(═O)—O—R₆,—CH₂—O—CH₃,—CH₂—OH, and —CN, wherein:
R₄ is H or small alkyl, and
R₅ is selected from alkyl, —(CH₂)₀₋₃-cycloalkyl, —(CH₂)₀₋₃-heterocycloalkyl, —(CH(alkyl)-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, and —(C(H)(heteroaryl))-alkyl;
or R₄ and R₅ together with the nitrogen atom to which they are attached can form a carbon-containing 5-, or 6-membered saturated heterocyclic ring, optionally containing an additional heteroatom selected from N, and O, which may be optionally mono- or di-substituted with substituents selected from oxo, alkyl, alkoxy, OH, halo and CF₃; and
R₆ is H, small alkyl, and —(CH₂)-phenyl; and
R₁ can be:
(i) Formula (IIb),

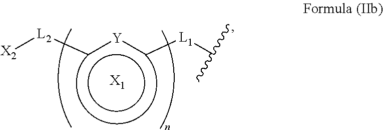

wherein:
n is 1;
L₁ is a linker selected from a single bond, —(CH₂)₁₋₃—, —(CH)(alkyl)-, and —C(alkyl)(alkyl)-; Y is selected from C, N, O and S;
X₁ is phenyl or a 5- or 6-membered carbon-containing aromatic ring containing one, two or three ring members that are selected from N, S, and O;
wherein X₁ may be optionally substituted with 1 or 2 substituents independently selected from alkyl, cycloalkyl, alkoxy, OH, CF₃, and halo;
L₂ is a linker selected from a single bond, —(CH₂)₁₋₃—, —(CH)(alkyl)-, —C(alkyl)(alkyl)-, —C(═O)—, and —SO₂—; and
X₂ is either:
(a) —NR₂OR₂₁, wherein R₂₀ is selected from H, alkyl, and —(CH₂)₀₋₃-R_{22;}
wherein R₂₁ is selected from H, alkyl, and —(CH₂)₀₋₃-R₂₃;

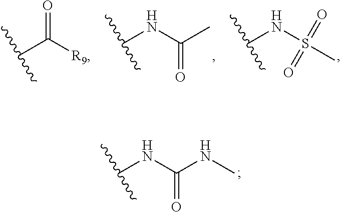

wherein R₂₂ is selected from cycloalkyl, heterocycloalkyl, aryl, heteroaryl,

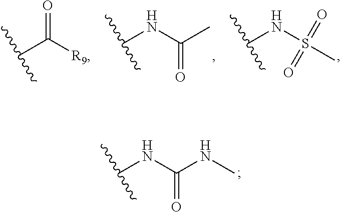

wherein R₂₃ is selected from cycloalkyl, heterocycloalkyl, aryl, heteroaryl, or
(b)₂-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected from alkyl, cycloalkyl, alkoxy, OH, Oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O; or
(ii) Formula (III) comprising an aromatic ring A fused to a non-aromatic ring A′,

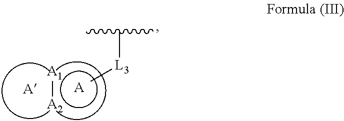

wherein:
L₃ is a single bond;
A₁ and Az are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R₇ is selected from alkyl, alkoxy, and —(CH₂)₀₋₃-R₈,

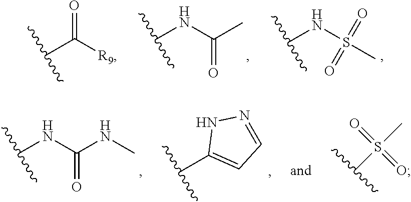

wherein R₈ is selected from cycloalkyl, heterocycloalkyl, wherein R₉ is selected from H, alkyl, OH, NH₂, and alkoxy;
wherein A′ may be optionally substituted with 1, 2, or 3 further substituents R₁₀, R₁₁, and R₁₇, wherein R₁₀, R₁₁, and R₁₇, are independently selected from alkyl, cycloalkyl, alkoxy, OH, oxo, CF₃, and halo, or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O; and
R₃ can be phenyl substituted with one or two substituents independently selected from small alkyl, small alkoxy, CF₃, halo, —C(═O)—NH₂, and CN.
B₄ can be N(R₂), B₅ can be N, and B₆ can be C(R₃), wherein B₄ is connected to B₅ via a single bond and B₅ is connected to B₆ via a double bond, or B₄ can be C(R₂), B₅ can be N, and B₆ can be N(R₃), wherein B₄ is connected to B₅ via a double bond and B₅ is connected to B₆ via a single bond; R₂ can be selected from —C(═O)—N(R₄)(R₅), —C(═O)—O—R₆,—CH₂—NH—S(═O)₂(cyclopropyl),—CH₂—O—CH₃,—CH₂—OH, and —CN, wherein:
R₄ is H or small alkyl, and
R₅ is selected from alkyl, —(CH₂)₀₋₃-cycloalkyl, —(CH₂)₀₋₃-heterocycloalkyl, —(CH(alkyl)-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, and —(C(H)(heteroaryl))-alkyl;
or R₄ and R₅ together with the nitrogen atom to which they are attached can form a carbon-containing 5-, or 6-membered saturated heterocyclic ring, optionally containing an additional heteroatom selected from N, and O, which may be optionally mono- or di-substituted with substituents selected from oxo, alkyl, alkoxy, OH, halo and CF₃; and
R₆ is H, small alkyl, and —(CH₂)-phenyl; and
R₁ can be:
(i) Formula (IIb),

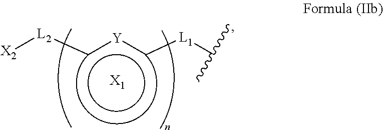

wherein:
n is 1;
L₁ is a linker selected from a single bond, —(CH₂)₁₋₃—, —(CH)(alkyl)-, and —C(alkyl)(alkyl)-; Y is selected from C, N, O and S;
X₁ is phenyl or a 5- or 6-membered carbon-containing aromatic ring containing one, two or three ring members that are selected from N, S, and O;
wherein X₁ may be optionally substituted with 1 or 2 substituents independently selected from alkyl, cycloalkyl, alkoxy, OH, CF₃, and halo;
L₂ is a linker selected from a single bond, —(CH₂)₁₋₃—, —(CH)(alkyl)-, —C(alkyl)(alkyl)-, —C(═O)—, and —SO₂—; and
X₂ is either:
(a) —NR₂OR₂₁, wherein R₂₀ is selected from H, alkyl, and —(CH₂)₀₋₃-R_{22;}
wherein Rz1 is selected from H, alkyl, and —(CH₂)₀₋₃-R₂₃;

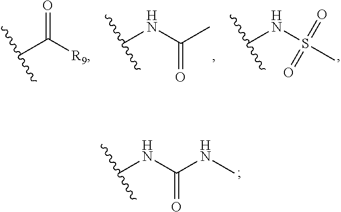

wherein R₂₂ is selected from cycloalkyl, heterocycloalkyl, aryl, heteroaryl,

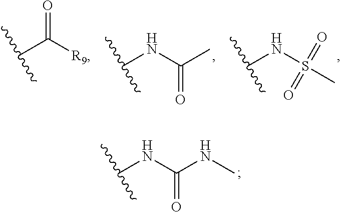

wherein R₂₃ is selected from cycloalkyl, heterocycloalkyl, aryl, heteroaryl, or
(b)₂-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O; or
(ii) Formula (III) comprising an aromatic ring A fused to a non-aromatic ring A′,

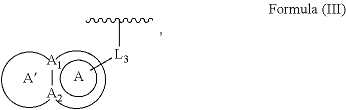

wherein:
L₃ is a single bond;
A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R₇ is selected from alkyl, alkoxy, and —(CH₂)₀₋₃-R₈,

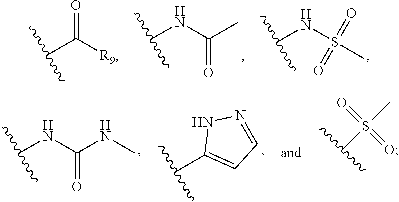

wherein R₈ is selected from cycloalkyl, heterocycloalkyl, wherein R₉ is selected from H, alkyl, OH, NH₂, and alkoxy;
wherein A′ may be optionally substituted with 1, 2, or 3 further substituents R₁₀, R₁₁, and R₁₇, wherein R₁₀, R₁₁, and R₁₇, are independently selected from alkyl, cycloalkyl, alkoxy, OH, oxo, CF₃, and halo, or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O; and R₃ can be phenyl substituted with one or two substituents independently selected from small alkyl, small alkoxy, CF₃, halo, —C(═O)—NH₂, and CN.
B₁ can be a carbon centre and B₂ can be a carbon centre, wherein B₃ is a single bond between B₂ and the nitrogen atom to which R₁ is attached and B₁ is connected to B₂ via a single bond, or B₁ can be a nitrogen centre and B₂ can be a carbon centre wherein B₁ is connected to B₂ via a double bond; B₄ can be N(R₂), B₅ can be N, and B₆ can be C(R₃), wherein B₄ is connected to B₅ via a single bond and B₅ is connected to B₆ via a double bond, or B₄ can be C(R₂), B₅ can be N, and B₆ can be N(R₃), wherein B₄ is connected to B₅ via a double bond and B₅ is connected to B₆ via a single bond;
R₂ can be selected from —C(═O)—N(R₄)(R₅), —C(═O)—O—R₆,—CH₂—NH—S(═O)₂(cyclopropyl),—CH₂—O—CH₃,—CH₂—OH, and —CN, wherein:
R₄ is H or small alkyl, and
R₅ is selected from alkyl, —(CH₂)₀₋₃-cycloalkyl, —(CH₂)₀₋₃-heterocycloalkyl, —(CH(alkyl))-heterocycloalkyl, —(CH₂)₀₋₃-polycycloalkyl, —(CH₂)₀₋₃-aryl, —(C(H)(phenyl)-alkyl, —(CH₂)₀₋₃heteroaryl, and —(C(H)(heteroaryl))-alkyl;
or R₄ and R₅ together with the nitrogen atom to which they are attached can form a carbon-containing 5-, or 6-membered saturated heterocyclic ring, optionally containing an additional heteroatom selected from N, and O, which may be optionally mono- or di-substituted with substituents selected from oxo, alkyl, alkoxy, OH, halo and CF₃; and
R₆ is H, small alkyl, and —(CH₂)-phenyl; and
R₁ can be:
(i) Formula (IIa),

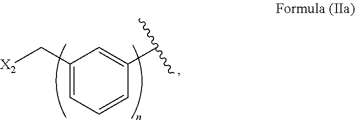

wherein:
n is 1;
L₁ is a linker selected from a single bond, —(CH₂)₁₋₃—, —(CH)(alkyl)-, and —C(alkyl)(alkyl)-; Y is selected from C, N, O and S;
X₁ is phenyl or a 5- or 6-membered carbon-containing aromatic ring containing one, two or three ring members that are selected from N, S, and O;
wherein X₁ may be optionally substituted with 1 or 2 substituents independently selected from alkyl, cycloalkyl, alkoxy, OH, CF₃, and halo;
L₂ is a linker selected from a single bond, —(CH₂)₁₋₃—, —(CH)(alkyl)-, —C(alkyl)(alkyl)-, —C(═O)—, and —SO₂—; and either:
wherein R₂₀ is selected from H, alkyl, and —(CH₂)₀₋₃-R_{2;}
wherein R₂₁ is selected from H, alkyl, and —(CH₂)₀₋₃-R_{23;}
X₂ is (a) —NR₂OR₂₁,

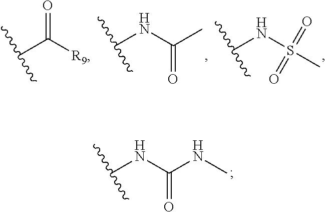

wherein R₂₂ is selected from cycloalkyl, heterocycloalkyl, aryl, heteroaryl,

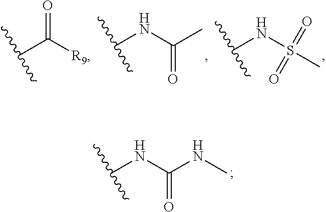

wherein R₂₃ is selected from cycloalkyl, heterocycloalkyl, aryl, heteroaryl, or
(b)₂-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O; or

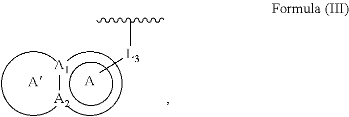

wherein:
L₃ is a single bond;
A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R₇ is selected from alkyl, alkoxy, and —(CH₂)₀₋₃-R₈, <CWU-Call number 932 wherein R₈ is selected from cycloalkyl, heterocycloalkyl,

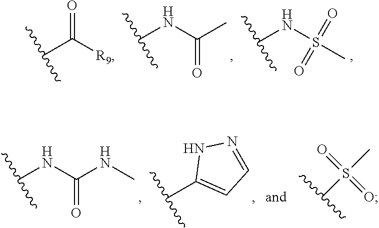

wherein R₉ is selected from H, alkyl, OH, NH₂, and alkoxy;
wherein A′ may be optionally substituted with 1, 2, or 3 further substituents R₁₀, R₁₁, and R₁₇, wherein R₁₀, R₁₁, and R₁₇, are independently selected from alkyl, cycloalkyl, alkoxy, OH, oxo, CF₃, and halo, or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O; and
R₃ can be phenyl substituted with one or two substituents independently selected from small alkyl, small alkoxy, CF₃, halo, —C(═O)—NH₂, and CN.
R₂ can be selected from —C(═O)—N(R₄)(R₅), —C(═O)—O—R₆,—CH₂—NH—S(═O)₂(cyclopropyl),—CH₂—O—CH₃,—CH₂—OH, and —CN, wherein:
R₄ is H or small alkyl, and
R₅ is selected from alkyl, —(CH₂)₀₋₃-cycloalkyl, —(CH₂)₀₋₃-heterocycloalkyl, —(CH(alkyl))-heterocycloalkyl, —(CH₂)₀₋₃-polycycloalkyl, —(CH₂)₀₋₃-aryl, —(C(H)(phenyl)-alkyl, —(CH₂)₀₋₃heteroaryl, and —(C(H)(heteroaryl))-alkyl;
or R₄ and R₅ together with the nitrogen atom to which they are attached can form a carbon-containing 5-, or 6-membered saturated heterocyclic ring, optionally containing an additional heteroatom selected from N, and O, which may be optionally mono- or di-substituted with substituents selected from oxo, alkyl, alkoxy, OH, halo and CF₃; and
R₆ is H, small alkyl, and —(CH₂)-phenyl; and
R₁ can be:
(i) Formula (IIb),

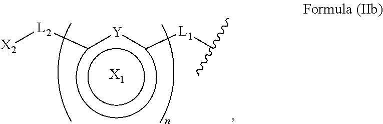

wherein:
n is 1;
L₁ is a linker selected from a single bond, —(CH₂)₁₋₃—, —(CH)(alkyl)-, and —C(alkyl)(alkyl)-; Y is selected from C, N, O and S;
X₁ is phenyl or a 5- or 6-membered carbon-containing aromatic ring containing one, two or three ring members that are selected from N, S, and O;
wherein X₁ may be optionally substituted with 1 or 2 substituents independently selected from alkyl, cycloalkyl, alkoxy, OH, CF₃, and halo;
L₂ is a linker selected from a single bond, —(CH₂)₁₋₃—, —(CH)(alkyl)-, —C(alkyl)(alkyl)-, —C(═O)—, and —

SO₂—; and
X₂ is either:
(a) —NR₂OR₂₁, wherein R₂₀ is selected from H, alkyl, and —(CH₂)₀₋₃-R_{22;}
wherein R₂₁ is selected from H, alkyl, and —(CH₂)₀₋₃-R₂₃;

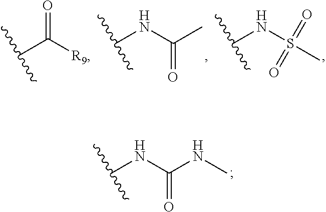

wherein R₂₂ is selected from cycloalkyl, heterocycloalkyl, aryl, heteroaryl,

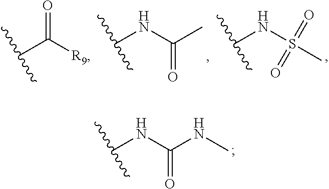

wherein R₂₃ is selected from cycloalkyl, heterocycloalkyl, aryl, heteroaryl, or
(b)₂-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected from alkyl, cycloalkyl, alkoxy, OH, Oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O; or
(ii) Formula (III) comprising an aromatic ring A fused to a non-aromatic ring A′,

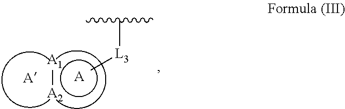

wherein:
L₃ is a single bond;
A₁ and Az are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R₇ is selected from alkyl, alkoxy, and —(CH₂)₀₋₃-R₈,

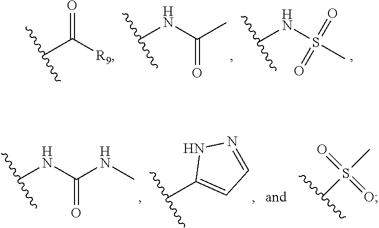

wherein R₈ is selected from cycloalkyl, heterocycloalkyl, wherein R₉ is selected from H, alkyl, OH, NH₂, and alkoxy;
wherein A′ may be optionally substituted with 1, 2, or 3 further substituents R₁₀, R₁₁, and R₁₇, wherein R₁₀, R₁₁, and R₁₇, are independently selected from alkyl, cycloalkyl, alkoxy, OH, oxo, CF₃, and halo, or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O; and
R₃ can be phenyl substituted with one or two substituents independently selected from small alkyl, small alkoxy, CF₃, halo, —C(═O)—NH₂, and CN.
B₁ can be a carbon centre and B₂ can be a carbon centre, wherein B₃ is a single bond between B₂ and the nitrogen atom to which R₁ is attached and B₁ is connected to B₂ via a single bond, and R₃ can be phenyl substituted with one or two halo substituents.
B₄ can be C(R₂), B₅ can be N, and B₆ can be N(R₃), wherein B₄ is connected to B₅ via a double bond and B₅ is connected to B₆ via a single bond, and R₃ can be phenyl substituted with one or two halo substituents.
B₁ can be a carbon centre and B₂ can be a carbon centre, wherein B₃ is a single bond between B₂ and the nitrogen atom to which R₁ is attached and B₁ is connected to B₂ via a single bond; B₄ can be C(R₂), B₅ can be N, and B₆ can be N(R₃), wherein B₄ is connected to B₅ via a double bond and B₅ is connected to B₆ via a single bond; and R₃ can be phenyl substituted with one or two halo substituents.
B₁ can be a carbon centre and B₂ can be a carbon centre, wherein B₃ is a single bond between B₂ and the nitrogen atom to which R₁ is attached and B₁ is connected to B₂ via a single bond; R₂ can be selected from —C(═O)—N(R₄)(R₅), —C(═O)—O—R₆,—CH₂—O—CH₃, and —CN; and R₃ can be phenyl substituted with one or two halo substituents.
B₄ can be C(R₂), B₅ can be N, and B₆ can be N(R₃), wherein B₄ is connected to B₅ via a double bond and B₅ is connected to B₆ via a single bond; R₂ can be selected from —C(═O)—N(R₄)(R₅), —C(═O)—O—R₆,—CH₂—O—CH₃, and —CN; and R₃ can be phenyl substituted with one or two halo substituents.
B₁ can be a carbon centre and B₂ can be a carbon centre, wherein B₃ is a single bond between B₂ and the nitrogen atom to which R₁ is attached and B₁ is connected to B₂ via a single bond; B₄ can be C(R₂), B₅ can be N, and B₆ can be N(R₃), wherein B₄ is connected to B₅ via a double bond and B₅ is connected to B₆ via a single bond; R₂ can be selected from —C(═O)—N(R₄)(R₅), —C(═O)—O—R₆,—CH₂—O—CH₃, and —CN; and R₃ can be phenyl substituted with one or two halo substituents.
B₁ can be a carbon centre and B₂ can be a carbon centre, wherein B₃ is a single bond between B₂ and the nitrogen atom to which R₁ is attached and B₁ is connected to B₂ via a single bond; R₂ can be selected from —C(═O)—N(R₄)(R₅), —C(═O)—O—R₆,—CH₂—O—CH₃, and —CN, wherein:
R₄ is H or small alkyl, and
R₅ is selected from alkyl, —(CH₂)₀₋₃-cycloalkyl, —(CH₂)₀₋₃-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, and —(C(H)(heteroaryl))-alkyl;
or R₄ and R₅ together with the nitrogen atom to which they are attached can form a carbon-containing 5-, or 6-membered saturated heterocyclic ring, optionally containing an additional heteroatom selected from N, and O, which may be optionally mono- or di-substituted with substituents selected from oxo, alkyl, alkoxy, OH, halo and CF₃; and
R₆ is H, small alkyl, and —(CH₂)-phenyl; and
R₃ can be phenyl substituted with one or two halo substituents.
B₄ can be C(R₂), B₅ can be N, and B₆ can be N(R₃), wherein B₄ is connected to B₅ via a double bond and B₅ is connected to B₆ via a single bond;
R₂ can be selected from —C(═O)—N(R₄)(R₅), —C(═O)—O—R₆,—CH₂—O—CH₃, and —CN, wherein:
R₄ is H or small alkyl, and
R₅ is selected from alkyl, —(CH₂)₀₋₃-cycloalkyl, —(CH₂)₀₋₃-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, and —(C(H)(heteroaryl))-alkyl;
or R₄ and R₅ together with the nitrogen atom to which they are attached can form a carbon-containing 5-, or 6-membered saturated heterocyclic ring, optionally containing an additional heteroatom selected from N, and O, which may be optionally mono- or di-substituted with substituents selected from oxo, alkyl, alkoxy, OH, halo and CF₃; and
R₆ is H, small alkyl, and —(CH₂)-phenyl; and
R₃ can be phenyl substituted with one or two halo substituents.
B₁ can be a carbon centre and B₂ can be a carbon centre, wherein B₃ is a single bond between B₂ and the nitrogen atom to which R₁ is attached and B₁ is connected to B₂ via a single bond; B₄ can be C(R₂), B₅ can be N, and B₆ can be N(R₃), wherein B₄ is connected to B₅ via a double bond and B₅ is connected to B₆ via a single bond;
R₂ can be selected from —C(═O)—N(R₄)(R₅), —C(═O)—O—R₆,—CH₂—O—CH₃, and —CN, wherein:
R₄ is H or small alkyl, and
R₅ is selected from alkyl, —(CH₂)₀₋₃-cycloalkyl, —(CH₂)₀₋₃-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, and —(C(H)(heteroaryl))-alkyl;
or R₄ and R₅ together with the nitrogen atom to which they are attached can form a carbon-containing 5-, or 6-membered saturated heterocyclic ring, optionally containing an additional heteroatom selected from N, and O, which may be optionally mono- or di-substituted with substituents selected from oxo, alkyl, alkoxy, OH, halo and CF₃; and
R₆ is H, small alkyl, and —(CH₂)-phenyl; and
R₃ can be phenyl substituted with one or two halo substituents.
B₁ can be a carbon centre and B₂ can be a carbon centre, wherein B₃ is a single bond between B₂ and the nitrogen atom to which R₁ is attached and B₁ is connected to B₂ via a single bond;
R₁ can be:
(i) Formula (IIa),

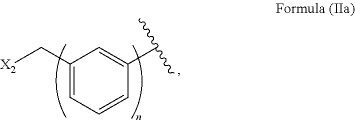

wherein:
n is 1;
X₂ is a non-aromatic, carbon-containing mono- or bi-cyclic ring containing 5, 6, or 7 ring members, wherein at least one ring member is independently selected from N, S, and O;
wherein X₂ may be optionally substituted with 1 or 2 substituents R₁₈ and R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O; or
(ii) Formula (III) comprising an aromatic ring A fused to a non-aromatic ring A′,

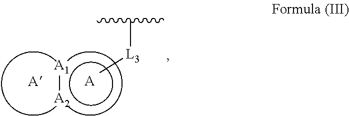

wherein:
L₃ is a single bond;
A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R₇ is selected from alkyl, alkoxy, and —(CH₂)₀₋₃-R₈,

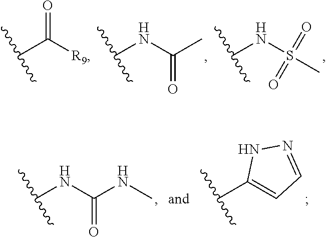

wherein R₈ is selected from cycloalkyl, heterocycloalkyl, wherein R₉ is selected from H, alkyl, OH, NH₂, and alkoxy;
wherein A′ may be optionally substituted with 1 or 2 further substituents R₁₀ and R₁₁, wherein R₁₀ and R₁₁ are independently selected from alkyl, cycloalkyl, alkoxy, OH, oxo, CF₃, and halo, or wherein A′ may be optionally substituted with 1 further substituent R₂₄ selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O; and
R₃ can be phenyl substituted with one or two halo substituents.
B₄ can be C(R₂), B₅ can be N, and B₆ can be N(R₃), wherein B₄ is connected to B₅ via a double bond and B₅ is connected to B₆ via a single bond;
R₁ can be:
(i) Formula (IIa),

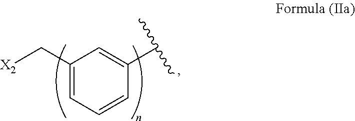

wherein:
n is 1;
X₂ is a non-aromatic, carbon-containing mono- or bi-cyclic ring containing 5, 6, or 7 ring members, wherein at least one ring member is independently selected from N, S, and O;
wherein X₂ may be optionally substituted with 1 or 2 substituents R₁₈ and R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O; or

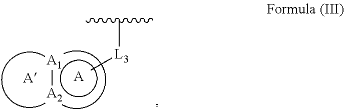

wherein:
L₃ is a single bond;
A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R₇ is selected from alkyl, alkoxy, and —(CH₂)₀₋₃-R₈,
wherein R₈ is selected from cycloalkyl, heterocycloalkyl,

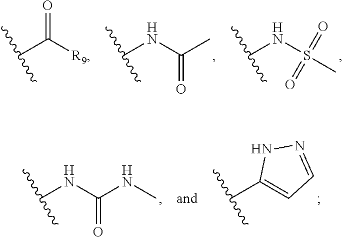

wherein R₉ is selected from H, alkyl, OH, NH₂, and alkoxy;
wherein A′ may be optionally substituted with 1 or 2 further substituents R₁₀ and R₁₁, wherein R₁₀ and R₁₁ are independently selected from alkyl, cycloalkyl, alkoxy, OH, oxo, CF₃, and halo, or wherein A′ may be optionally substituted with 1 further substituent R₂₄ selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O; and
R₃ can be phenyl substituted with one or two halo substituents.
B₁ can be a carbon centre and B₂ can be a carbon centre, wherein B₃ is a single bond between B₂ and the nitrogen atom to which R₁ is attached and B₁ is connected to B₂ via a single bond, and B₄ can be C(R₂), B₅ can be N, and B₆ can be N(R₃), wherein B₄ is connected to B₅ via a double bond and B₅ is connected to B₆ via a single bond;

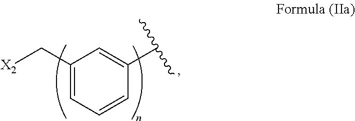

wherein:
n is 1;
X₂ is a non-aromatic, carbon-containing mono- or bi-cyclic ring containing 5, 6, or 7 ring members, wherein at least one ring member is independently selected from N, S, and O;
wherein X₂ may be optionally substituted with 1 or 2 substituents R₁₈ and R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O; or
(ii) Formula (III) comprising an aromatic ring A fused to a non-aromatic ring A′,

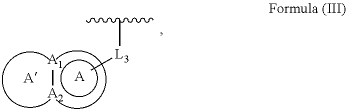

wherein:
L₃ is a single bond;
A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R₇ is selected from alkyl, alkoxy, and —(CH₂)₀₋₃-R₈,

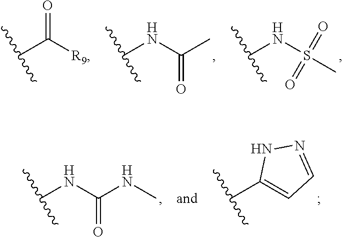

wherein R₈ is selected from cycloalkyl, heterocycloalkyl, wherein R₉ is selected from H, alkyl, OH, NH₂, and alkoxy;
wherein A′ may be optionally substituted with 1 or 2 further substituents R₁₀ and R₁₁, wherein R₁₀ and R₁₁ are independently selected from alkyl, cycloalkyl, alkoxy, OH, oxo, CF₃, and halo, or wherein A′ may be optionally substituted with 1 further substituent R₂₄ selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O; and
R₃ can be phenyl substituted with one or two halo substituents.
B₁ can be a carbon centre and B₂ can be a carbon centre, wherein B₃ is a single bond between B₂ and the nitrogen atom to which R₁ is attached and B₁ is connected to B₂ via a single bond; R₂ can be selected from —C(═O)—N(R₄)(R₅), —C(═O)—O—R₆,—CH₂—O—CH₃, and —CN;
R₁ can be:
(i) Formula (IIa),

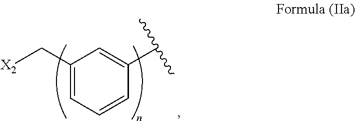

wherein:
n is 1;
X₂ is a non-aromatic, carbon-containing mono- or bi-cyclic ring containing 5, 6, or 7 ring members, wherein at least one ring member is independently selected from N, S, and O;
wherein X₂ may be optionally substituted with 1 or 2 substituents R₁₈ and R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O; or
(ii) Formula (III) comprising an aromatic ring A fused to a non-aromatic ring A′,

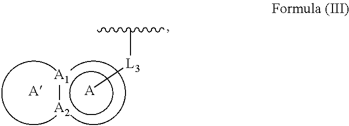

wherein:
L₃ is a single bond;
A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R₇ is selected from alkyl, alkoxy, and —(CH₂)₀₋₃-R₈,

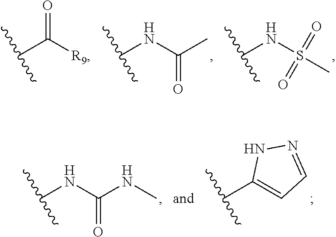

wherein R₈ is selected from cycloalkyl, heterocycloalkyl, wherein R₉ is selected from H, alkyl, OH, NH₂, and alkoxy;
wherein A′ may be optionally substituted with 1 or 2 further substituents R₁₀ and R₁₁, wherein R₁₀ and R₁₁ are independently selected from alkyl, cycloalkyl, alkoxy, OH, oxo, CF₃, and halo, or wherein A′ may be optionally substituted with 1 further substituent R₂₄ selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O; and
R₃ can be phenyl substituted with one or two halo substituents.
B₄ can be C(R₂), Bs can be N, and B₆ can be N(R₃), wherein B₄ is connected to Bs via a double bond and Bs is connected to B₆ via a single bond; R₂ can be selected from —C(═O)—N(R₄)(R₅), —C(═O)—O—R₆,—CH₂—O—CH₃, and —CN;
R₁ can be:
(i) Formula (IIa),

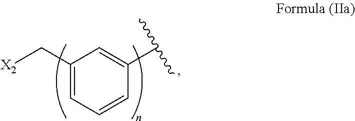

wherein:
n is 1;
X₂ is a non-aromatic, carbon-containing mono- or bi-cyclic ring containing 5, 6, or 7 ring members, wherein at least one ring member is independently selected from N, S, and O;
wherein X₂ may be optionally substituted with 1 or 2 substituents R₁₈ and R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O; or
(ii) Formula (III) comprising an aromatic ring A fused to a non-aromatic ring A′,

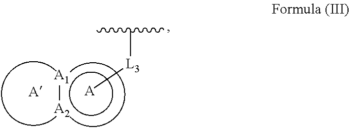

wherein:
L₃ is a single bond;
A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R₇ is selected from alkyl, alkoxy, and —(CH₂)₀₋₃-R₈,

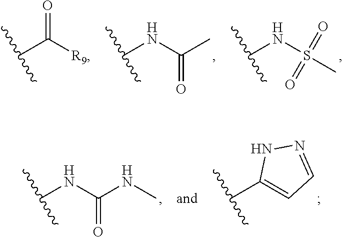

wherein R₈ is selected from cycloalkyl, heterocycloalkyl, wherein R₉ is selected from H, alkyl, OH, NH₂, and alkoxy;
wherein A′ may be optionally substituted with 1 or 2 further substituents R₁₀ and R₁₁, wherein R₁₀ and R₁₁ are independently selected from alkyl, cycloalkyl, alkoxy, OH, oxo, CF₃, and halo, or wherein A′ may be optionally substituted with 1 further substituent R₂₄ selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O; and
R₃ can be phenyl substituted with one or two halo substituents.
B₁ can be a carbon centre and B₂ can be a carbon centre, wherein B₃ is a single bond between B₂ and the nitrogen atom to which R₁ is attached and B₁ is connected to B₂ via a single bond; B₄ can be C(R₂), B₅ can be N, and B₆ can be N(R₃), wherein B₄ is connected to B₅ via a double bond and B₅ is connected to B₆ via a single bond; R₂ can be selected from —C(═O)—N(R₄)(R₅), —C(═O)—O—R₆,—CH₂—O—CH₃, and —CN;

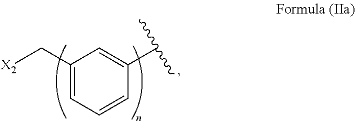

wherein:
n is 1;
X₂ is a non-aromatic, carbon-containing mono- or bi-cyclic ring containing 5, 6, or 7 ring members, wherein at least one ring member is independently selected from N, S, and O;
wherein X₂ may be optionally substituted with 1 or 2 substituents R₁₈ and R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O; or
(ii) Formula (III) comprising an aromatic ring A fused to a non-aromatic ring A′,

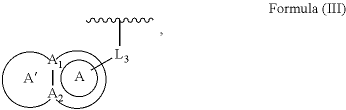

wherein:
L₃ is a single bond;
A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R₇ is selected from alkyl, alkoxy, and —(CH₂)₀₋₃-R₈, wherein R₈ is selected from cycloalkyl, heterocycloalkyl,

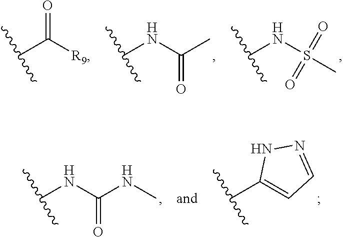

wherein R₉ is selected from H, alkyl, OH, NH₂, and alkoxy;
wherein A′ may be optionally substituted with 1 or 2 further substituents R₁₀ and R₁₁, wherein R₁₀ and R₁₁ are independently selected from alkyl, cycloalkyl, alkoxy, OH, oxo, CF₃, and halo, or wherein A′ may be optionally substituted with 1 further substituent R₂₄ selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O; and
R₃ can be phenyl substituted with one or two halo substituents.
B₁ can be a carbon centre and B₂ can be a carbon centre, wherein B₃ is a single bond between B₂ and the nitrogen atom to which R₁ is attached and B₁ is connected to B₂ via a single bond;
R₂ can be selected from —C(═O)—N(R₄)(R₅), —C(═O)—O—R₆,—CH₂—O—CH₃, and —CN, wherein:
R₄ is H or small alkyl, and
R₅ is selected from alkyl, —(CH₂)₀₋₃-cycloalkyl, —(CH₂)₀₋₃-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, and —(C(H)(heteroaryl))-alkyl;
or R₄ and R₅ together with the nitrogen atom to which they are attached can form a carbon-containing 5-, or 6-membered saturated heterocyclic ring, optionally containing an additional heteroatom selected from N, and O, which may be optionally mono- or di-substituted with substituents selected from oxo, alkyl, alkoxy, OH, halo and CF₃; and
R₆ is H, small alkyl, and —(CH₂)-phenyl;
R₁ can be:

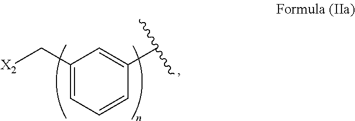

wherein:
n is 1;
X₂ is a non-aromatic, carbon-containing mono- or bi-cyclic ring containing 5, 6, or 7 ring members, wherein at least one ring member is independently selected from N, S, and O;
wherein X₂ may be optionally substituted with 1 or 2 substituents R₁₈ and R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O; or
(ii) Formula (III) comprising an aromatic ring A fused to a non-aromatic ring A′,

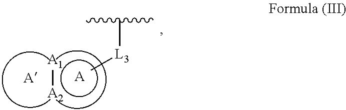

wherein:
L₃ is a single bond;
A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R, is selected from alkyl, alkoxy, and —(CH₂)₀₋₃-R₈,

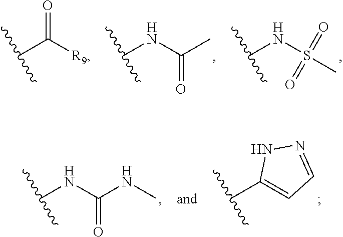

wherein R₈ is selected from cycloalkyl, heterocycloalkyl, wherein R₉ is selected from H, alkyl, OH, NH₂, and alkoxy;
wherein A′ may be optionally substituted with 1 or 2 further substituents R₁₀ and R₁₁, wherein R₁₀ and R₁₁ are independently selected from alkyl, cycloalkyl, alkoxy, OH, oxo, CF₃, and halo, or wherein A′ may be optionally substituted with 1 further substituent R₂₄ selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O; and
R₃ can be phenyl substituted with one or two halo substituents.
B₄ can be C(R₂), B₅ can be N, and B₆ can be N(R₃), wherein B₄ is connected to B₅ via a double bond and B₅ is connected to B₆ via a single bond;
R₂ can be selected from —C(═O)—N(R₄)(R₅), —C(═O)—O—R₆,—CH₂—O—CH₃, and —CN, wherein:
R₄ is H or small alkyl, and
R₅ is selected from alkyl, —(CH₂)₀₋₃-cycloalkyl, —(CH₂)₀₋₃-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, and —(C(H)(heteroaryl))-alkyl;
or R₄ and R₅ together with the nitrogen atom to which they are attached can form a carbon-containing 5-, or 6-membered saturated heterocyclic ring, optionally containing an additional heteroatom selected from N, and O, which may be optionally mono- or di-substituted with substituents selected from oxo, alkyl, alkoxy, OH, halo and CF₃; and
Ro is H, small alkyl, and —(CH₂)-phenyl; and
R₁ can be:

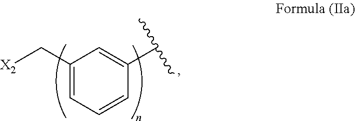

wherein:
n is 1;
X₂ is a non-aromatic, carbon-containing mono- or bi-cyclic ring containing 5, 6, or 7 ring members, wherein at least one ring member is independently selected from N, S, and O;
wherein X₂ may be optionally substituted with 1 or 2 substituents R₁₈ and R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O; or
(ii) Formula (III) comprising an aromatic ring A fused to a non-aromatic ring A′,

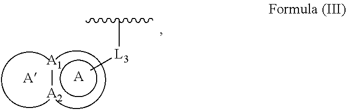

wherein:
L₃ is a single bond;
A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R₇ is selected from alkyl, alkoxy, and —(CH₂)₀₋₃-R₈,

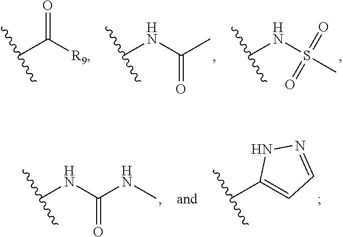

wherein R₈ is selected from cycloalkyl, heterocycloalkyl, wherein R₈ is selected from H, alkyl, OH, NH₂, and alkoxy;
wherein A′ may be optionally substituted with 1 or 2 further substituents R₁₀ and R₁₁, wherein R₁₀ and R₁₁ are independently selected from alkyl, cycloalkyl, alkoxy, OH, oxo, CF₃, and halo, or wherein A′ may be optionally substituted with 1 further substituent R₂₄ selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O; and
R₃ can be phenyl substituted with one or two halo substituents.
B₁ can be a carbon centre and B₂ can be a carbon centre, wherein B₃ is a single bond between B₂ and the nitrogen atom to which R₁ is attached and B₁ is connected to B₂ via a single bond; B₄ can be C(R₂), B₅ can be N, and B₆ can be N(R₃), wherein B₄ is connected to B₅ via a double bond and B₅ is connected to B₆ via a single bond;
R₂ can be selected from —C(═O)—N(R₄)(R₅), —C(═O)—O—R₆,—CH₂—O—CH₃, and —CN, wherein:
R₄ is H or small alkyl, and
R₅ is selected from alkyl, —(CH₂)₀₋₃-cycloalkyl, —(CH₂)₀₋₃-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, and —(C(H)(heteroaryl))-alkyl;
or R₄ and R₅ together with the nitrogen atom to which they are attached can form a carbon-containing 5-, or 6-membered saturated heterocyclic ring, optionally containing an additional heteroatom selected from N, and O, which may be optionally mono- or di-substituted with substituents selected from oxo, alkyl, alkoxy, OH, halo and CF₃; and
R₆ is H, small alkyl, and —(CH₂)-phenyl;

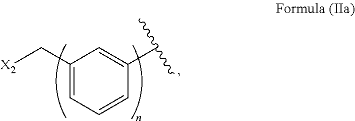

wherein:
n is 1;
X₂ is a non-aromatic, carbon-containing mono- or bi-cyclic ring containing 5, 6, or 7 ring members, wherein at least one ring member is independently selected from N, S, and O;
wherein X₂ may be optionally substituted with 1 or 2 substituents R₁₈ and R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O; or
(ii) Formula (III) comprising an aromatic ring A fused to a non-aromatic ring A′,

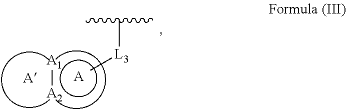

wherein:
L₃ is a single bond;
A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R, is selected from alkyl, alkoxy, and —(CH₂)₀₋₃-R₈,

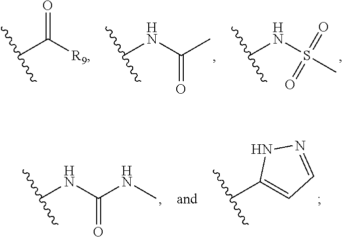

wherein R₈ is selected from cycloalkyl, heterocycloalkyl, wherein R₉ is selected from H, alkyl, OH, NH₂, and alkoxy;
wherein A′ may be optionally substituted with 1 or 2 further substituents R₁₀ and R₁₁, wherein R₁₀ and R₁₁ are independently selected from alkyl, cycloalkyl, alkoxy, OH, oxo, CF₃, and halo, or wherein A′ may be optionally substituted with 1 further substituent R₂₄ selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O; and
R₃ can be phenyl substituted with one or two halo substituents.
R₂ can be selected from —C(═O)—N(R₄)(R₅), —C(═O)—O—R₆,—CH₂—O—CH₃, and —CN; and R₃ can be phenyl substituted with one or two halo substituents.
R₂ can be selected from —C(═O)—N(R₄)(R₅), —C(═O)—O—R₆,—CH₂—O—CH₃, and —CN, wherein:
R₄ is H or small alkyl, and
R₅ is selected from alkyl, —(CH₂)₀₋₃-cycloalkyl, —(CH₂)₀₋₃-heterocycloalkyl, —(CH₂)₀₋₃-polycycloalkyl, —(CH₂)₀₋₃-aryl, - (C(H)(phenyl)-alkyl, —(CH₂)₀₋₃heteroaryl, and —(C(H)(heteroaryl)-alkyl;
or R₄ and R₅ together with the nitrogen atom to which they are attached can form a carbon-containing 5-, or 6-membered saturated heterocyclic ring, optionally containing an additional heteroatom selected from N, and O, which may be optionally mono- or di-substituted with substituents selected from oxo, alkyl, alkoxy, OH, halo and CF₃; and R₆ is H, small alkyl, and —(CH₂)-phenyl; and
R₃ can be phenyl substituted with one or two halo substituents.

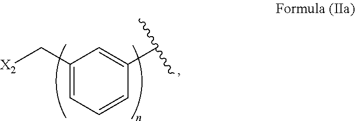

wherein:
n is 1;
X₂ is a non-aromatic, carbon-containing mono- or bi-cyclic ring containing 5, 6, or 7 ring members, wherein at least one ring member is independently selected from N, S, and O;
wherein X₂ may be optionally substituted with 1 or 2 substituents R₁₈ and R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O; or
(ii) Formula (III) comprising an aromatic ring A fused to a non-aromatic ring A′,

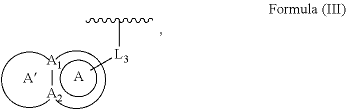

wherein:
L₃ is a single bond;
A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R₇ is selected from alkyl, alkoxy, and —(CH₂)₀₋₃-R₈, wherein R₈ is selected from cycloalkyl, heterocycloalkyl

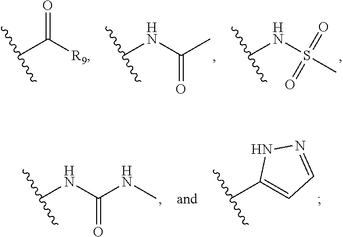

wherein R₉ is selected from H, alkyl, OH, NH₂, and alkoxy;
wherein A’ may be optionally substituted with 1 or 2 further substituents R₁₀ and R₁₁, wherein R₁₀ and R₁₁ are independently selected from alkyl, cycloalkyl, alkoxy, OH, oxo, CF₃, and halo, or wherein A′ may be optionally substituted with 1 further substituent R₂₄ selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O; and
R₃ can be phenyl substituted with one or two halo substituents.
R₂ can be selected from —C(═O)—N(R₄)(R₅), —C(═O)—O—R₆,—CH₂—O—CH₃, and —CN;
R₁ can be:
(i) Formula (IIa),

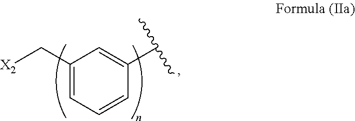

wherein:
n is 1;
X₂ is a non-aromatic, carbon-containing mono- or bi-cyclic ring containing 5, 6, or 7 ring members, wherein at least one ring member is independently selected from N, S, and O;
wherein X₂ may be optionally substituted with 1 or 2 substituents R₁₈ and R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O; or

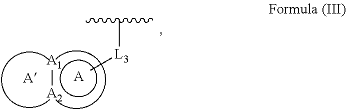

wherein:
L₃ is a single bond;
A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R₇ is selected from alkyl, alkoxy, and —(CH₂)₀₋₃-R₈,

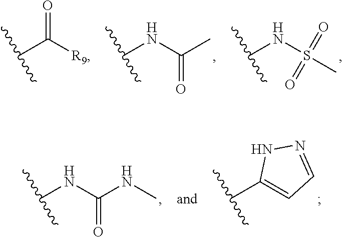

wherein R₈ is selected from cycloalkyl, heterocycloalkyl, wherein R₉ is selected from H, alkyl, OH, NH₂, and alkoxy;
wherein A′ may be optionally substituted with 1 or 2 further substituents R₁₀ and R₁₁, wherein R₁₀ and R₁₁ are independently selected from alkyl, cycloalkyl, alkoxy, OH, oxo, CF₃, and halo, or wherein A′ may be optionally substituted with 1 further substituent R₂₄ selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O; and
R₃ can be phenyl substituted with one or two halo substituents.
R₂ can be selected from —C(═O)—N(R₄)(R₅), —C(═O)—O—R₆,—CH₂—O—CH₃, and —CN, wherein:
R₄ is H or small alkyl, and
R₅ is selected from alkyl, —(CH₂)₀₋₃-cycloalkyl, —(CH₂)₀₋₃-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, and —(C(H)(heteroaryl))-alkyl;
or R₄ and R₅ together with the nitrogen atom to which they are attached can form a carbon-containing 5-, or 6-membered saturated heterocyclic ring, optionally containing an additional heteroatom selected from N, and O, which may be optionally mono- or di-substituted with substituents selected from oxo, alkyl, alkoxy, OH, halo and CF₃; and
R₆ is H, small alkyl, and —(CH₂)-phenyl;
R₁ can be:
(i) Formula (IIa),

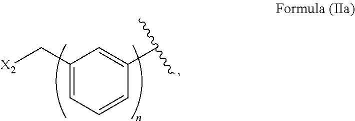

wherein:
n is 1;
X₂ is a non-aromatic, carbon-containing mono- or bi-cyclic ring containing 5, 6, or 7 ring members, wherein at least one ring member is independently selected from N, S, and O;
wherein X₂ may be optionally substituted with 1 or 2 substituents R₁₈ and R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O; or

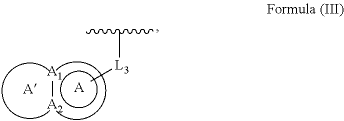

wherein:
L₃ is a single bond;
A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R₇ is selected from alkyl, alkoxy, and —(CH₂)₀₋₃-R₈,

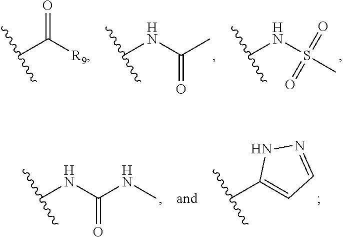

wherein R₈ is selected from cycloalkyl, heterocycloalkyl, wherein R₉ is selected from H, alkyl, OH, NH₂, and alkoxy;
wherein A′ may be optionally substituted with 1 or 2 further substituents R₁₀ and R₁₁, wherein R₁₀ and R₁₁ are independently selected from alkyl, cycloalkyl, alkoxy, OH, oxo, CF₃, and halo, or wherein A′ may be optionally substituted with 1 further substituent R₂₄ selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O; and
R₃ can be phenyl substituted with one or two halo substituents.
B₁ can be a carbon centre and B₂ can be a carbon centre, wherein B₃ is a single bond between B₂ and the nitrogen atom to which R₁ is attached and B₁ is connected to B₂ via a single bond, and R₃ can be selected

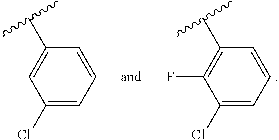 [SMILES: *c1cccc(Cl)c1 ; *c1cccc(Cl)c1F]

from:
B₄ can be C(R₂), Bs can be N, and B₆ can be N(R₃), wherein B₄ is connected to Bs via a double bond and B₅

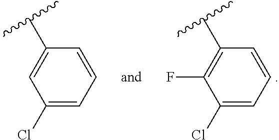 [SMILES: *c1cccc(Cl)c1 ; *c1cccc(Cl)c1F]

is connected to B₆ via a single bond, and R₃ can be selected from:
B₁ can be a carbon centre and B₂ can be a carbon centre, wherein B₃ is a single bond between B₂ and the nitrogen atom to which R₁ is attached and B₁ is connected to B₂ via a single bond; B₄ can be C(R₂), Bs can be N, and B₆ can be N(R₃), wherein B₄ is connected to B₅ via a double bond and B₅ is connected to B₆ via a

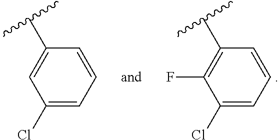 [SMILES: *c1cccc(Cl)c1 ; *c1cccc(Cl)c1F]

single bond; and R₃ can be selected from:
B₁ can be a carbon centre and B₂ can be a carbon centre, wherein B₃ is a single bond between B₂ and the nitrogen atom to which R₁ is attached and B₁ is connected to B₂ via a single bond; R₂ can be selected from

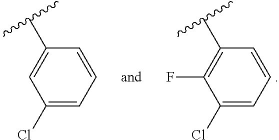 [SMILES: *c1cccc(Cl)c1 ; *c1cccc(Cl)c1F]

—C(═O)—N(R₄)(R₅), —C(═O)—O—R₆,—CH₂—O—CH₃, and —CN; and R₃ can be selected from:
B₄ can be C(R₂), Bs can be N, and B₆ can be N(R₃), wherein B₄ is connected to Bs via a double bond and B₅ is connected to B₆ via a single bond; R₂ can be selected from —C(═O)—N(R₄)(R₅), —C(═O)—O—R₆,—CH₂—O—CH₃,

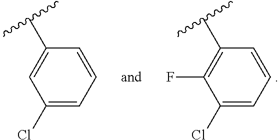 [SMILES: *c1cccc(Cl)c1 ; *c1cccc(Cl)c1F]

and —CN; and R₃ can be selected from:
B₁ can be a carbon centre and B₂ can be a carbon centre, wherein B₃ is a single bond between B₂ and the nitrogen atom to which R₁ is attached and B₁ is connected to B₂ via a single bond; B₄ can be C(R₂), Bs can be N, and B₆ can be N(R₃), wherein B₄ is connected to B₅ via a double bond and B₅ is connected to B₆ via a single bond; R₂ can be selected from —C(═O)—N(R₄)(R₅), —C(═O)—O—R₆,—CH₂—O—CH₃, and —CN; R₃ can be selected

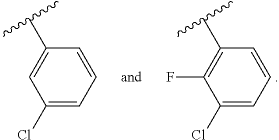 [SMILES: *c1cccc(Cl)c1 ; *c1cccc(Cl)c1F]

from:
B₁ can be a carbon centre and B₂ can be a carbon centre, wherein B₃ is a single bond between B₂ and the nitrogen atom to which R₁ is attached and B₁ is connected to B₂ via a single bond;
R₂ can be selected from —C(═O)—N(R₄)(R₅), —C(═O)—O—R₆,—CH₂—O—CH₃, and —CN, wherein:
R₄ is H or small alkyl, and
R₅ is selected from alkyl, —(CH₂)₀₋₃-cycloalkyl, —(CH₂)₀₋₃-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, and —(C(H)(heteroaryl))-alkyl;
or R₄ and R₅ together with the nitrogen atom to which they are attached can form a carbon-containing 5-, or 6-membered saturated heterocyclic ring, optionally containing an additional heteroatom selected from N, and O, which may be optionally mono- or di-substituted with substituents selected from oxo, alkyl, alkoxy, OH, halo and CF₃; and
R₆ is H, small alkyl, and —(CH₂)-phenyl; and

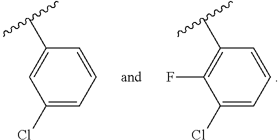 [SMILES: *c1cccc(Cl)c1 ; *c1cccc(Cl)c1F]

R₃ can be selected from:
B₄ can be C(R₂), B₅ can be N, and B₆ can be N(R₃), wherein B₄ is connected to B₅ via a double bond and B₅ is connected to B₆ via a single bond;
R₂ can be selected from —C(═O)—N(R₄)(R₅), —C(═O)—O—R₆,—CH₂—O—CH₃, and —CN, wherein:
R₄ is H or small alkyl, and
R₅ is selected from alkyl, —(CH₂)₀₋₃-cycloalkyl, —(CH₂)₀₋₃-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, and —(C(H)(heteroaryl))-alkyl;
or R₄ and R₅ together with the nitrogen atom to which they are attached can form a carbon-containing 5-, or 6-membered saturated heterocyclic ring, optionally containing an additional heteroatom selected from N, and O, which may be optionally mono- or di-substituted with substituents selected from oxo, alkyl, alkoxy, OH, halo and CF₃; and R₆ is H, small alkyl, and —(CH₂)-phenyl; and

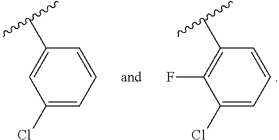 [SMILES: *c1cccc(Cl)c1 ; *c1cccc(Cl)c1F]

R₃ can be selected from:
B₁ can be a carbon centre and B₂ can be a carbon centre, wherein B₃ is a single bond between B₂ and the nitrogen atom to which R₁ is attached and B₁ is connected to B₂ via a single bond; B₄ can be C(R₂), B₅ can be N, and B₆ can be N(R₃), wherein B₄ is connected to B₅ via a double bond and B₅ is connected to B₆ via a single bond;
R₂ can be selected from —C(═O)—N(R₄)(R₅), —C(═O)—O—R₆,—CH₂—O—CH₃, and —CN, wherein:
R₄ is H or small alkyl, and
R₅ is selected from alkyl, —(CH₂)₀₋₃-cycloalkyl, —(CH₂)₀₋₃-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, and —(C(H)(heteroaryl))-alkyl;
or R₄ and R₅ together with the nitrogen atom to which they are attached can form a carbon-containing 5-, or 6-membered saturated heterocyclic ring, optionally containing an additional heteroatom selected from N, and O, which may be optionally mono- or di-substituted with substituents selected from oxo, alkyl, alkoxy, OH, halo and CF₃; and
R₆ is H, small alkyl, and —(CH₂)-phenyl;

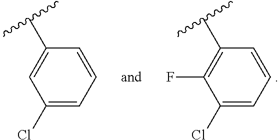 [SMILES: *c1cccc(Cl)c1 ; *c1cccc(Cl)c1F]

R₃ can be selected from:
B₁ can be a carbon centre and B₂ can be a carbon centre, wherein B₃ is a single bond between B₂ and the nitrogen atom to which R₁ is attached and B₁ is connected to B₂ via a single bond;
R₁ can be:

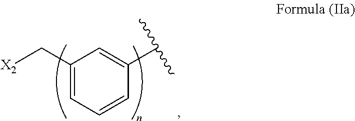

wherein:
n is 1;
X₂ is a non-aromatic, carbon-containing mono- or bi-cyclic ring containing 5, 6, or 7 ring members, wherein at least one ring member is independently selected from N, S, and O;
wherein X₂ may be optionally substituted with 1 or 2 substituents R₁₈ and R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O; or
(ii) Formula (III) comprising an aromatic ring A fused to a non-aromatic ring A′,

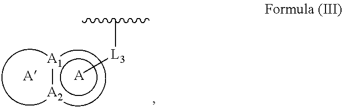

wherein:
L₃ is a single bond;
A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R₇ is selected from alkyl, alkoxy, and —(CH₂)₀₋₃-R₈,

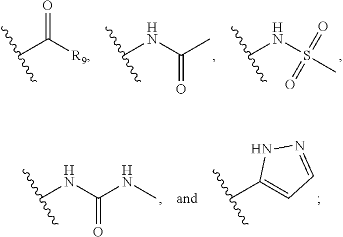

wherein R₈ is selected from cycloalkyl, heterocycloalkyl, wherein R₈ is selected from H, alkyl, OH, NH₂, and alkoxy;
wherein A′ may be optionally substituted with 1 or 2 further substituents R₁₀ and R₁₁, wherein R₁₀ and R₁₁ are independently selected from alkyl, cycloalkyl, alkoxy, OH, oxo, CF₃, and halo, or wherein A′ may be optionally substituted with 1 further substituent R₂₄ selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O; and

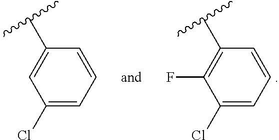 [SMILES: *c1cccc(Cl)c1 ; *c1cccc(Cl)c1F]

R₃ can be selected from:
B₄ can be C(R₂), Bs can be N, and B₆ can be N(R₃), wherein B₄ is connected to Bs via a double bond and B₅ is connected to B₆ via a single bond;
R₁ can be:
(i) Formula (IIa),

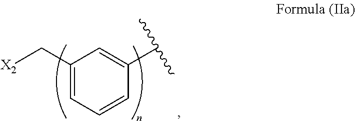

wherein:
n is 1; X₂ is a non-aromatic, carbon-containing mono- or bi-cyclic ring containing 5, 6, or 7 ring members, wherein at least one ring member is independently selected from N, S, and O;
wherein X₂ may be optionally substituted with 1 or 2 substituents R₁₈ and R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O; or
(ii) Formula (III) comprising an aromatic ring A fused to a non-aromatic ring A′,

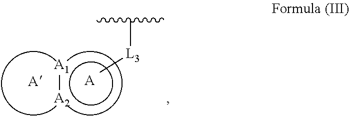

wherein:
L₃ is a single bond;
A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R₇ is selected from alkyl, alkoxy, and —(CH₂)₀₋₃-R₈,

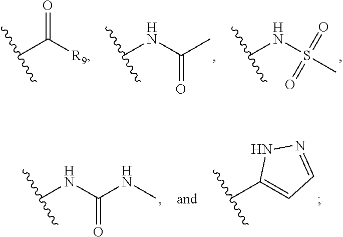

wherein R₈ is selected from cycloalkyl, heterocycloalkyl, wherein R₈ is selected from H, alkyl, OH, NH₂, and alkoxy;
wherein A′ may be optionally substituted with 1 or 2 further substituents R₁₀ and R₁₁, wherein R₁₀ and R₁₁ are independently selected from alkyl, cycloalkyl, alkoxy, OH, oxo, CF₃, and halo, or wherein A′ may be optionally substituted with 1 further substituent R₂₄ selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O; and

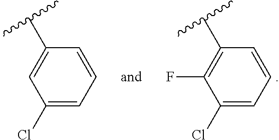 [SMILES: *c1cccc(Cl)c1 ; *c1cccc(Cl)c1F]

R₃ can be selected from:
B₁ can be a carbon centre and B₂ can be a carbon centre, wherein B₃ is a single bond between B₂ and the nitrogen atom to which R₁ is attached and B₁ is connected to B₂ via a single bond, and B₄ can be C(R₂), B₅ can be N, and B₆ can be N(R₃), wherein B₄ is connected to B₅ via a double bond and B₅ is connected to B₆ via a single bond;
R₁ can be:
(i) Formula (IIa),

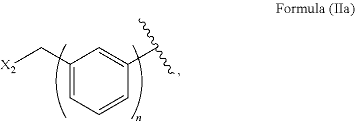

wherein:
n is 1;
X₂ is a non-aromatic, carbon-containing mono- or bi-cyclic ring containing 5, 6, or 7 ring members, wherein at least one ring member is independently selected from N, S, and O;
wherein X₂ may be optionally substituted with 1 or 2 substituents R₁₈ and R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O; or

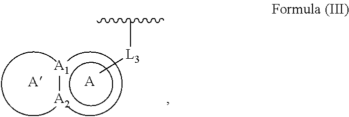

wherein:
L₃ is a single bond;
A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R₇ is selected from alkyl, alkoxy, and —(CH₂)₀₋₃-R₈,

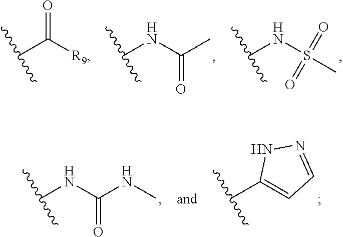

wherein R₈ is selected from cycloalkyl, heterocycloalkyl, wherein R₉ is selected from H, alkyl, OH, NH₂, and alkoxy;
wherein A′ may be optionally substituted with 1 or 2 further substituents R₁₀ and R₁₁, wherein R₁₀ and R₁₁ are independently selected from alkyl, cycloalkyl, alkoxy, OH, oxo, CF₃, and halo, or wherein A′ may be optionally substituted with 1 further substituent R₂₄ selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O; and

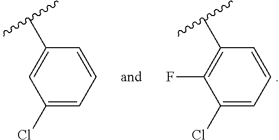 [SMILES: *c1cccc(Cl)c1 ; *c1cccc(Cl)c1F]

R₃ can be selected from:
B₁ can be a carbon centre and B₂ can be a carbon centre, wherein B₃ is a single bond between B₂ and the nitrogen atom to which R₁ is attached and B₁ is connected to B₂ via a single bond; R₂ can be selected from —C(═O)—N(R₄)(R₅), —C(═O)—O—R₆,—CH₂—O—CH₃, and —CN;
R₁ can be:
(i) Formula (IIa),

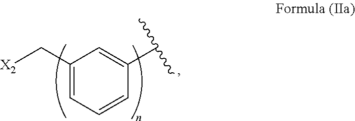

wherein:
n is 1;
X₂ is a non-aromatic, carbon-containing mono- or bi-cyclic ring containing 5, 6, or 7 ring members, wherein at least one ring member is independently selected from N, S, and O;
wherein X₂ may be optionally substituted with 1 or 2 substituents R₁₈ and R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O; or
(ii) Formula (III) comprising an aromatic ring A fused to a non-aromatic ring A′,

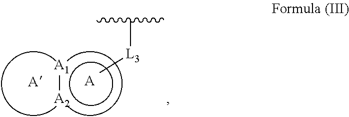

wherein:
L₃ is a single bond;
A₁ and Az are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R₇ is selected from alkyl, alkoxy, and —(CH₂)₀₋₃—R₉,

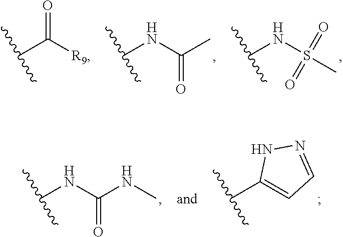

wherein R₈ is selected from cycloalkyl, heterocycloalkyl, wherein R₉ is selected from H, alkyl, OH, NH₂, and alkoxy;
wherein A′ may be optionally substituted with 1 or 2 further substituents R₁₀ and R₁₁, wherein R₁₀ and Ru are independently selected from alkyl, cycloalkyl, alkoxy, OH, oxo, CF₃, and halo, or wherein A′ may be optionally substituted with 1 further substituent R₂₄ selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O; and

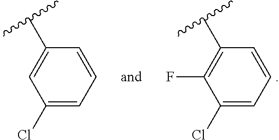 [SMILES: *c1cccc(Cl)c1 ; *c1cccc(Cl)c1F]

R₃ can be selected from:
B₄ can be C(R₂), B₅ can be N, and B₆ can be N(R₃), wherein B₄ is connected to B₅ via a double bond and B₅ is connected to B₆ via a single bond; R₂ can be selected from —C(═O)—N(R₄)(R₅), —C(═O)—O—R₆,—CH₂—O—CH₃, and —CN;
R₁ can be:
(i) Formula (IIa),

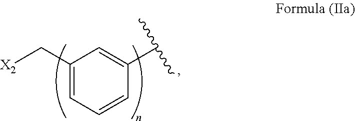

wherein:
n is 1;
X₂ is a non-aromatic, carbon-containing mono- or bi-cyclic ring containing 5, 6, or 7 ring members, wherein at least one ring member is independently selected from N, S, and O;
wherein X₂ may be optionally substituted with 1 or 2 substituents R₁₈ and R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O; or
(ii) Formula (III) comprising an aromatic ring A fused to a non-aromatic ring A′,

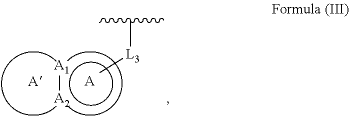

wherein:
L₃ is a single bond;
A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R₇ is selected from alkyl, alkoxy, and —(CH₂)₀₋₃-R₈,

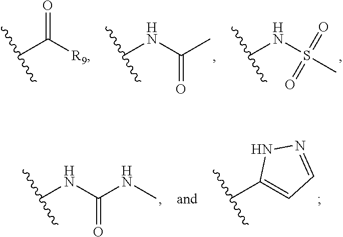

wherein R₈ is selected from cycloalkyl, heterocycloalkyl, wherein R₉ is selected from H, alkyl, OH, NH₂, and alkoxy;
wherein A′ may be optionally substituted with 1 or 2 further substituents R₁₀ and R₁₁, wherein R₁₀ and R₁₁ are independently selected from alkyl, cycloalkyl, alkoxy, OH, oxo, CF₃, and halo, or wherein A′ may be optionally substituted with 1 further substituent R₂₄ selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O; and

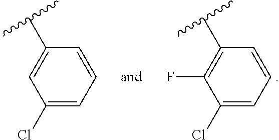 [SMILES: *c1cccc(Cl)c1 ; *c1cccc(Cl)c1F]

R₃ can be selected from:
B₁ can be a carbon centre and B₂ can be a carbon centre, wherein B₃ is a single bond between B₂ and the nitrogen atom to which R₁ is attached and B₁ is connected to B₂ via a single bond; B₄ can be C(R₂), B₅ can be N, and B₆ can be N(R₃), wherein B₄ is connected to B₅ via a double bond and B₅ is connected to B₆ via a single bond; R₂ can be selected from —C(═O)—N(R₄)(R₅), —C(═O)—O—R₆,—CH₂—O—CH₃, and —CN; R₁ can be:
(i) Formula (IIa),

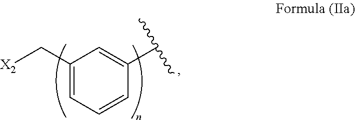

wherein:
n is 1;
X₂ is a non-aromatic, carbon-containing mono- or bi-cyclic ring containing 5, 6, or 7 ring members, wherein at least one ring member is independently selected from N, S, and O;
wherein X₂ may be optionally substituted with 1 or 2 substituents R₁₈ and R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O; or

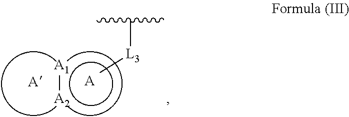

wherein:
L₃ is a single bond;
A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R, is selected from alkyl, alkoxy, and —(CH₂)₀₋₃-R₈,

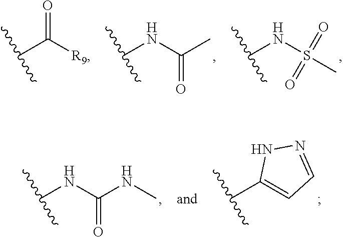

wherein R₈ is selected from cycloalkyl, heterocycloalkyl, wherein R₉ is selected from H, alkyl, OH, NH₂, and alkoxy;
wherein A′ may be optionally substituted with 1 or 2 further substituents R₁₀ and R₁₁, wherein R₁₀ and Ru are independently selected from alkyl, cycloalkyl, alkoxy, OH, oxo, CF₃, and halo, or wherein A′ may be optionally substituted with 1 further substituent R₂₄ selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O; and

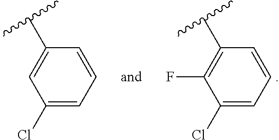 [SMILES: *c1cccc(Cl)c1 ; *c1cccc(Cl)c1F]

R₃ can be selected from:
B₁ can be a carbon centre and B₂ can be a carbon centre, wherein B₃ is a single bond between B₂ and the nitrogen atom to which R₁ is attached and B₁ is connected to B₂ via a single bond;
R₂ can be selected from —C(═O)—N(R₄)(R₅), —C(═O)—O—R₆,—CH₂—O—CH₃, and —CN, wherein:
R₄ is H or small alkyl, and
R₅ is selected from alkyl, —(CH₂)₀₋₃-cycloalkyl, —(CH₂)₀₋₃-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, and —(C(H)(heteroaryl))-alkyl;
or R₄ and R₅ together with the nitrogen atom to which they are attached can form a carbon-containing 5-, or 6-membered saturated heterocyclic ring, optionally containing an additional heteroatom selected from N, and O, which may be optionally mono- or di-substituted with substituents selected from oxo, alkyl, alkoxy, OH, halo and CF₃; and
R₆ is H, small alkyl, and —(CH₂)-phenyl;
R₁ can be:
(i) Formula (IIa),

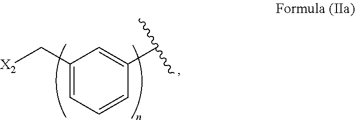

wherein:
n is 1;
X₂ is a non-aromatic, carbon-containing mono- or bi-cyclic ring containing 5, 6, or 7 ring members, wherein at least one ring member is independently selected from N, S, and O;
wherein X₂ may be optionally substituted with 1 or 2 substituents R₁₈ and R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O; or

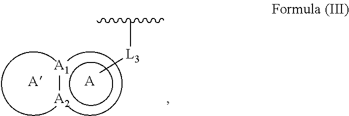

wherein:
L₃ is a single bond;
A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R₇ is selected from alkyl, alkoxy, and —(CH₂)₀₋₃-R₈,

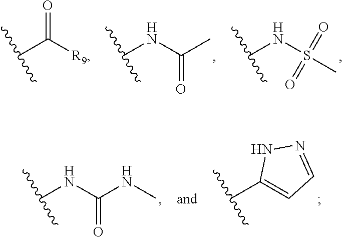

wherein R₈ is selected from cycloalkyl, heterocycloalkyl, wherein R₉ is selected from H, alkyl, OH, NH₂, and alkoxy;
wherein A′ may be optionally substituted with 1 or 2 further substituents R₁₀ and R₁₁, wherein R₁₀ and Ru are independently selected from alkyl, cycloalkyl, alkoxy, OH, oxo, CF₃, and halo, or wherein A′ may be optionally substituted with 1 further substituent R₂₄ selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O; and

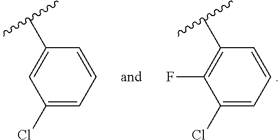 [SMILES: *c1cccc(Cl)c1 ; *c1cccc(Cl)c1F]

R₃ can be selected from:
B₄ can be C(R₂), B₅ can be N, and B₆ can be N(R₃), wherein B₄ is connected to B₅ via a double bond and B₅ is connected to B₆ via a single bond;
R₂ can be selected from —C(═O)—N(R₄)(R₅), —C(═O)—O—R₆,—CH₂—O—CH₃, and —CN, wherein:
R₄ is H or small alkyl, and
R₅ is selected from alkyl, —(CH₂)₀₋₃-cycloalkyl, —(CH₂)₀₋₃-heterocycloalkyl, —(CH₂)₀₋₃-polycycloalkyl, —(CH₂)₀₋₃-aryl, —(C(H)(phenyl)-alkyl, —(CH₂)₀₋₃heteroaryl, and —(C(H)(heteroaryl)-alkyl;
or R₄ and R₅ together with the nitrogen atom to which they are attached can form a carbon-containing 5-, or 6-membered saturated heterocyclic ring, optionally containing an additional heteroatom selected from N, and O, which may be optionally mono- or di-substituted with substituents selected from oxo, alkyl, alkoxy, OH, halo and CF₃; and R₆ is H, small alkyl, and —(CH₂)-phenyl;
R₁ can be:
(i) Formula (IIa),

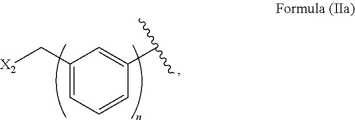

wherein:
n is 1;
X₂ is a non-aromatic, carbon-containing mono- or bi-cyclic ring containing 5, 6, or 7 ring members, wherein at least one ring member is independently selected from N, S, and O;
wherein X₂ may be optionally substituted with 1 or 2 substituents R₁₈ and R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O; or

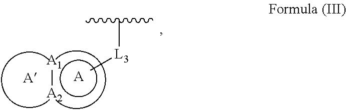

wherein:
L₃ is a single bond;
A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R₇ is selected from alkyl, alkoxy, and —(CH₂)₀₋₃-R₈,

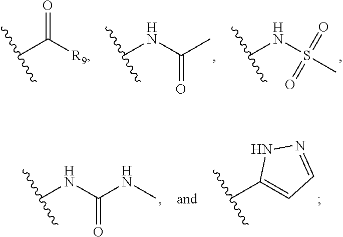

wherein R₈ is selected from cycloalkyl, heterocycloalkyl, wherein R₉ is selected from H, alkyl, OH, NH₂, and alkoxy;
wherein A′ may be optionally substituted with 1 or 2 further substituents R₁₀ and R₁₁, wherein R₁₀ and Ru are independently selected from alkyl, cycloalkyl, alkoxy, OH, oxo, CF₃, and halo, or wherein A′ may be optionally substituted with 1 further substituent R₂₄ selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O; and

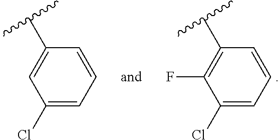 [SMILES: *c1cccc(Cl)c1 ; *c1cccc(Cl)c1F]

R₃ can be selected from:
B₁ can be a carbon centre and B₂ can be a carbon centre, wherein B₃ is a single bond between B₂ and the nitrogen atom to which R₁ is attached and B₁ is connected to B₂ via a single bond; B₄ can be C(R₂), B₅ can be N, and B₆ can be N(R₃), wherein B₄ is connected to B₅ via a double bond and B₅ is connected to B₆ via a single bond;
R₂ can be selected from —C(═O)—N(R₄)(R₅), —C(═O)—O—R₆,—CH₂—O—CH₃, and —CN, wherein:
R₄ is H or small alkyl, and
R₅ is selected from alkyl, —(CH₂)₀₋₃-cycloalkyl, —(CH₂)₀₋₃-heterocycloalkyl, —(CH₂)₀₋₃-polycycloalkyl, —(CH₂)₀₋₃-aryl, —(C(H)(phenyl)-alkyl, —(CH₂)₀₋₃heteroaryl, and —(C(H)(heteroaryl)-alkyl;
or R₄ and R₅ together with the nitrogen atom to which they are attached can form a carbon-containing 5-, or 6-membered saturated heterocyclic ring, optionally containing an additional heteroatom selected from N, and O, which may be optionally mono- or di-substituted with substituents selected from oxo, alkyl, alkoxy, OH, halo and CF₃; and
R₆ is H, small alkyl, and —(CH₂)-phenyl;
R₁ can be:
(i) Formula (IIa),

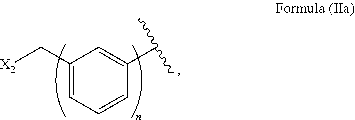

wherein:
n is 1;
X₂ is a non-aromatic, carbon-containing mono- or bi-cyclic ring containing 5, 6, or 7 ring members, wherein at least one ring member is independently selected from N, S, and O;
wherein X₂ may be optionally substituted with 1 or 2 substituents R₁₈ and R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O; or
(ii) Formula (III) comprising an aromatic ring A fused to a non-aromatic ring A′,

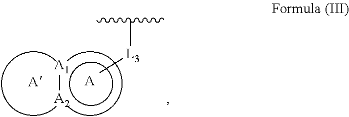

wherein:
L₃ is a single bond;
A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R₇ is selected from alkyl, alkoxy, and —(CH₂)₀₋₃-R₈, wherein R₈ is selected from cycloalkyl, heterocycloalkyl,

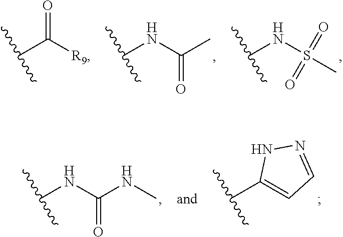

wherein R₉ is selected from H, alkyl, OH, NH₂, and alkoxy;
wherein A′ may be optionally substituted with 1 or 2 further substituents R₁₀ and R₁₁, wherein R₁₀ and R₁₁ are independently selected from alkyl, cycloalkyl, alkoxy, OH, oxo, CF₃, and halo, or wherein A′ may be optionally substituted with 1 further substituent R₂₄ selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O; and

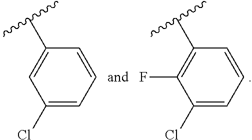 [SMILES: *c1cccc(Cl)c1 ; *c1cccc(Cl)c1F]

R₃ can be selected from:
R₂ can be selected from —C(═O)—N(R₄)(R₅), —C(═O)—O—R₆,—CH₂—O—CH₃, and —CN; and R₃ can be selected from:

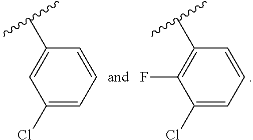 [SMILES: *c1cccc(Cl)c1 ; *c1cccc(Cl)c1F]

R₂ can be selected from —C(═O)—N(R₄)(R₅), —C(═O)—O—R₆,—CH₂—O—CH₃, and —CN, wherein:
R₄ is H or small alkyl, and
R₅ is selected from alkyl, —(CH₂)₀₋₃-cycloalkyl, —(CH₂)₀₋₃-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, and —(C(H)(heteroaryl))-alkyl;
or R₄ and R₅ together with the nitrogen atom to which they are attached can form a carbon-containing 5-, or 6-membered saturated heterocyclic ring, optionally containing an additional heteroatom selected from N, and O, which may be optionally mono- or di-substituted with substituents selected from oxo, alkyl, alkoxy, OH, halo and CF₃; and
R₃ is H, small alkyl, and —(CH₂)-phenyl; and

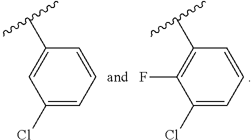 [SMILES: *c1cccc(Cl)c1 ; *c1cccc(Cl)c1F]

R₃ can be selected from:

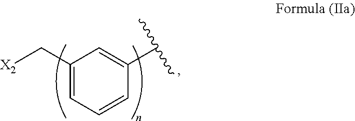

wherein:
n is 1;
X₂ is a non-aromatic, carbon-containing mono- or bi-cyclic ring containing 5, 6, or 7 ring members, wherein at least one ring member is independently selected from N, S, and O;
wherein X₂ may be optionally substituted with 1 or 2 substituents R₁₈ and R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O; or
(ii) Formula (III) comprising an aromatic ring A fused to a non-aromatic ring A′,

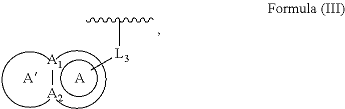

wherein:
L₃ is a single bond;
A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein Ry is selected from alkyl, alkoxy, and —(CH₂)₀₋₃-R₈,

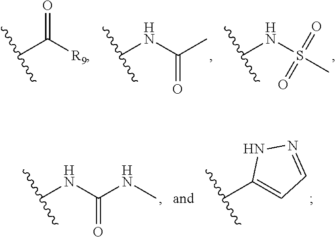

wherein R₈ is selected from cycloalkyl, heterocycloalkyl, wherein R₉ is selected from H, alkyl, OH, NH₂, and alkoxy;
wherein A′ may be optionally substituted with 1 or 2 further substituents R₁₀ and R₁₁, wherein R₁₀ and R₁₁ are independently selected from alkyl, cycloalkyl, alkoxy, OH, oxo, CF₃, and halo, or wherein A′ may be optionally substituted with 1 further substituent R₂₄ selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O; and

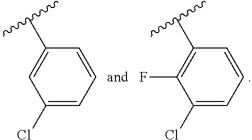 [SMILES: *c1cccc(Cl)c1 ; *c1cccc(Cl)c1F]

R₃ can be selected from:
R₂ can be selected from —C(═O)—N(R₄)(R₅), —C(═O)—O—R₆,—CH₂—O—CH₃, and —CN;
R₁ can be:
(i) Formula (IIa),

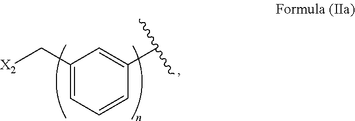

wherein:
n is 1;
X₂ is a non-aromatic, carbon-containing mono- or bi-cyclic ring containing 5, 6, or 7 ring members, wherein at least one ring member is independently selected from N, S, and O;
wherein X₂ may be optionally substituted with 1 or 2 substituents R₁₈ and R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O; or
(ii) Formula (III) comprising an aromatic ring A fused to a non-aromatic ring A′,

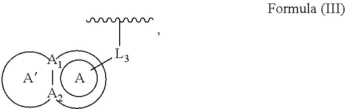

wherein:
L₃ is a single bond;
A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, CF₃, and halo;

A′ is a non-aromatic carbon-containing monocyclic ring containing 5, 6, or 7, ring members, wherein at least one ring member is independently selected from N, S, and O;
wherein A′ may be optionally substituted with one R₇, wherein R₇ is selected from alkyl, alkoxy, and —(CH₂)₀₋₃-R₈, wherein R₈ is selected from cycloalkyl, heterocycloalkyl,

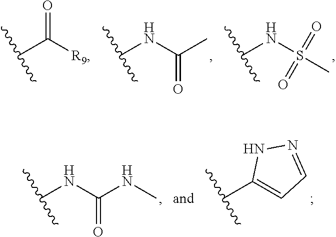

wherein R₉ is selected from H, alkyl, OH, NH₂, and alkoxy;
wherein A′ may be optionally substituted with 1 or 2 further substituents R₁₀ and R₁₁, wherein R₁₀ and R₁₁ are independently selected from alkyl, cycloalkyl, alkoxy, OH, oxo, CF₃, and halo, or wherein A′ may be optionally substituted with 1 further substituent R₂₄ selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O; and

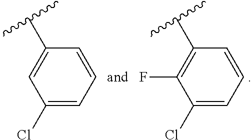 [SMILES: *c1cccc(Cl)c1 ; *c1cccc(Cl)c1F]

R₃ can be selected from:
R₂ can be selected from —C(═O)—N(R₄)(R₅), —C(═O)—O—R₆,—CH₂—O—CH₃, and —CN, wherein:
R₄ is H or small alkyl, and
R₅ is selected from alkyl, —(CH₂)₀₋₃-cycloalkyl, —(CH₂)₀₋₃-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, and —(C(H)(heteroaryl))-alkyl;
or R₄ and R₅ together with the nitrogen atom to which they are attached can form a carbon-containing 5-, or 6-membered saturated heterocyclic ring, optionally containing an additional heteroatom selected from N, and O, which may be optionally mono- or di-substituted with substituents selected from oxo, alkyl, alkoxy, OH, halo and CF₃; and
   R₆ is H, small alkyl, and —(CH₂)-phenyl;
R₁ can be:
(i) Formula (IIa),

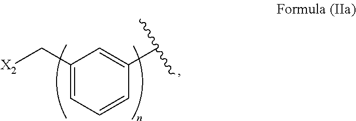

wherein:
n is 1;
X₂ is a non-aromatic, carbon-containing mono- or bi-cyclic ring containing 5, 6, or 7 ring members, wherein at least one ring member is independently selected from N, S, and O;
wherein X₂ may be optionally substituted with 1 or 2 substituents R₁₈ and R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O; or

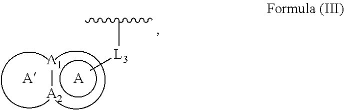

wherein:
L₃ is a single bond;
A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, CF₃, and halo;

A′ is a non-aromatic carbon-containing monocyclic ring containing 5, 6, or 7, ring members,
wherein at least one ring member is independently selected from N, S, and O;
wherein A′ may be optionally substituted with one R₇, wherein R₇ is selected from alkyl, alkoxy, and —(CH₂)₀₋₃—R₈,
wherein R₈ is selected from cycloalkyl, heterocycloalkyl,

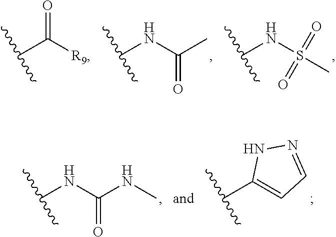

wherein R₉ is selected from H, alkyl, OH, NH₂, and alkoxy;
wherein A′ may be optionally substituted with 1 or 2 further substituents R₁₀ and R₁₁, wherein R₁₀ and Ru are independently selected from alkyl, cycloalkyl, alkoxy, OH, oxo, CF₃, and halo, or wherein A′ may be optionally substituted with 1 further substituent R₂₄ selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O; and
R₃ can be selected from:

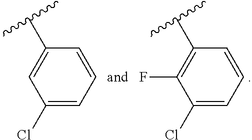 [SMILES: *c1cccc(Cl)c1 ; *c1cccc(Cl)c1F]

The compound, or a tautomer, isomer, stereoisomer (including an enantiomer, a diastereoisomer and a racemic and scalemic mixture thereof), a deuterated analogue, and a pharmaceutically acceptable salt and/or solvate thereof, can inhibit FXIIa.

FXIIa inhibition may be determined by the assay set out below (“Determination of FXIIa inhibition” in the “Biological Methods” section, i.e. using an IC₅₀ assay based on standard literature methods (see e.g Baeriswyl et al., ACS Chem. Biol., 2015, 10 (8) 1861; Bouckaert et al., European Journal of Medicinal Chemistry 110 (2016) 181)), such as for the data provided in Table 7. FXIIa inhibition is measured by determining IC₅₀ (nM).

The compound, or a tautomer, isomer, stereoisomer (including an enantiomer, a diastereoisomer and a racemic and scalemic mixture thereof), a deuterated analogue, and a pharmaceutically acceptable salt and/or solvate thereof, can inhibit FXIIa with an IC₅₀ value of <40,000 nM.

The compound, or a tautomer, isomer, stereoisomer (including an enantiomer, a diastereoisomer and a racemic and scalemic mixture thereof), a deuterated analogue, and a pharmaceutically acceptable salt and/or solvate thereof, can inhibit FXIIa with an IC₅₀ value of <10,000 nM.

The compound, or a tautomer, isomer, stereoisomer (including an enantiomer, a diastereoisomer and a racemic and scalemic mixture thereof), a deuterated analogue, and a pharmaceutically acceptable salt and/or solvate thereof, can inhibit FXIIa with an IC₅₀ value of <3,000 nM.

The compound, or a tautomer, isomer, stereoisomer (including an enantiomer, a diastereoisomer and a racemic and scalemic mixture thereof), a deuterated analogue, and a pharmaceutically acceptable salt and/or solvate thereof, can inhibit FXIIa with an IC₅₀ value of <1,000 nM.

The compound, or a tautomer, isomer, stereoisomer (including an enantiomer, a diastereoisomer and a racemic and scalemic mixture thereof), a deuterated analogue, and a pharmaceutically acceptable salt and/or solvate thereof, can inhibit FXIIa with an IC₅₀ value of <300 nM.

The compound, or a tautomer, isomer, stereoisomer (including an enantiomer, a diastereoisomer and a racemic and scalemic mixture thereof), a deuterated analogue, and a pharmaceutically acceptable salt and/or solvate thereof, can inhibit FXIIa with an IC₅₀ value of <100 nM.

The compound, or a tautomer, isomer, stereoisomer (including an enantiomer, a diastereoisomer and a racemic and scalemic mixture thereof), a deuterated analogue, and a pharmaceutically acceptable salt and/or solvate thereof, can inhibit FXIIa selectively compared to inhibition of any other serine protease.

It will be understood that inhibiting FXIIa selectivity compared to inhibition of other serine protease means that the IC₅₀ for inhibition of FXIIa is lower than the IC₅₀ for inhibition of any other serine protease.

The compound, or a tautomer, isomer, stereoisomer (including an enantiomer, a diastereoisomer and a racemic and scalemic mixture thereof), a deuterated analogue, and a pharmaceutically acceptable salt and/or solvate thereof, can inhibit FXIIa selectively compared to inhibition of any one of PKaI, KLK1, plasmin, FXa, FXIa, or thrombin.

It will be understood that inhibiting FXIIa selectivity compared to inhibition of any one of PKaI, KLK1, plasmin, FXa, FXIa, or thrombin means that the IC₅₀ for inhibition of FXIIa is lower than the IC₅₀ for inhibition of any one of PKaI, KLK1, plasmin, FXa, FXIa, or thrombin.

Inhibition of any one of PKaI, KLK1, plasmin, FXa, FXIa, or thrombin may be determined by the assay set out below (“Determination of related protease inhibition” in the “Biological Methods” section, i.e. using an IC₅₀ assay based on standard literature methods (see e.g. Shori et al., Biochem. Pharmacol., 1992,43, 1209; Bouckaert et al., European Journal of Medicinal Chemistry 110 (2016) 181)), such as for the data provided in Table 8. Inhibition of any one of PKaI, KLK1, plasmin, FXa, FXIa, or thrombin is measured by determining IC₅₀ (nM).

The compound, or a tautomer, isomer, stereoisomer (including an enantiomer, a diastereoisomer and a racemic and scalemic mixture thereof), a deuterated analogue, and a pharmaceutically acceptable salt and/or solvate thereof, can inhibit FXIIa selectively compared to inhibition of FXa.

The compound, or a tautomer, isomer, stereoisomer (including an enantiomer, a diastereoisomer and a racemic and scalemic mixture thereof), a deuterated analogue, and a pharmaceutically acceptable salt and/or solvate thereof, can be at least 10-fold selective for inhibition of FXIIa compared to inhibition of FXa.

The compound, or a tautomer, isomer, stereoisomer (including an enantiomer, a diastereoisomer and a racemic and scalemic mixture thereof), a deuterated analogue, and a pharmaceutically acceptable salt and/or solvate thereof, can be at least 100-fold selective for inhibition of FXIIa compared to inhibition of FXa.

The compound, or a tautomer, isomer, stereoisomer (including an enantiomer, a diastereoisomer and a racemic and scalemic mixture thereof), a deuterated analogue, and a pharmaceutically acceptable salt and/or solvate thereof, can be at least 1,000-fold selective for inhibition of FXIIa compared to inhibition of FXa.

The compound, or a tautomer, isomer, stereoisomer (including an enantiomer, a diastereoisomer and a racemic and scalemic mixture thereof), a deuterated analogue, and a pharmaceutically acceptable salt and/or solvate thereof, can be at least 10,000-fold selective for inhibition of FXIIa compared to inhibition of FXa.

More specifically, the invention provides compounds of formula (I), selected from Table 1a, Table 1b,

Table 1c, Table 1d, Table 2a, Table 2b, Table 2c, Table 2d, Table 3a, Table 3b, Table 3c, Table 3d, and Table 4, or a pharmaceutically acceptable salt, solvate, or solvate of a salt thereof.

The invention provides compounds of formula (I), selected from Table 1a, Table 1b, Table 1c, Table 2a, Table 2b, Table 2c, Table 3a, Table 3b, Table 3c, and Table 4, or a pharmaceutically acceptable salt, solvate, or solvate of a salt thereof.

The invention also provides compounds of formula (I), selected from Table 1a, Table 1b, Table 2a, Table 2b, Table 3a, Table 3b, and Table 4, or a pharmaceutically acceptable salt, solvate, or solvate of a salt thereof.

The invention also provides compounds of formula (I), selected from Table 1a, Table 2a, and Table 3a, or a pharmaceutically acceptable salt, solvate, or solvate of a salt thereof.

For the compounds provided in Table 1a, Table 1b, Table 1c, Table 2a, Table 2b, Table 3a, Table 3b, Table 3c, and Table 4, below, where stereochemistry is indicated, the compound is intended to cover all possible stereoisomers thereof.

The present invention therefore provides the compounds below in Table 1a, and pharmaceutically acceptable salts and/or solvates thereof. The present invention therefore also provides stereoisomers of the compounds below in Table 1a, and pharmaceutically acceptable salts and/or solvates thereof.

The present invention therefore provides the compounds below in Table 1b, and pharmaceutically acceptable salts and/or solvates thereof. The present invention therefore also provides stereoisomers of the compounds below in Table 1b, and pharmaceutically acceptable salts and/or solvates thereof.

The present invention therefore provides the compounds below in Table 1c, and pharmaceutically acceptable salts and/or solvates thereof. The present invention therefore also provides stereoisomers of the compounds below in Table 1c, and pharmaceutically acceptable salts and/or solvates thereof.

The present invention therefore provides the compounds below in Table 1d, and pharmaceutically acceptable salts and/or solvates thereof. The present invention therefore also provides stereoisomers of the compounds below in Table 1d, and pharmaceutically acceptable salts and/or solvates thereof.

The present invention therefore provides the compounds below in Table 2a, and pharmaceutically acceptable salts and/or solvates thereof. The present invention therefore also provides stereoisomers of the compounds below in Table 2a, and pharmaceutically acceptable salts and/or solvates thereof.

The present invention therefore provides the compounds below in Table 2b, and pharmaceutically acceptable salts and/or solvates thereof. The present invention therefore also provides stereoisomers of the compounds below in Table 2b, and pharmaceutically acceptable salts and/or solvates thereof.

The present invention therefore provides the compounds below in Table 2c, and pharmaceutically acceptable salts and/or solvates thereof. The present invention therefore also provides stereoisomers of the compounds below in Table 2c, and pharmaceutically acceptable salts and/or solvates thereof.

The present invention therefore provides the compounds below in Table 2d, and pharmaceutically acceptable salts and/or solvates thereof. The present invention therefore also provides stereoisomers of the compounds below in Table 2d, and pharmaceutically acceptable salts and/or solvates thereof.

The present invention therefore provides the compounds below in Table 3a, and pharmaceutically acceptable salts and/or solvates thereof. The present invention therefore also provides stereoisomers of the compounds below in Table 3a, and pharmaceutically acceptable salts and/or solvates thereof.

The present invention therefore provides the compounds below in Table 3b, and pharmaceutically acceptable salts and/or solvates thereof. The present invention therefore also provides stereoisomers of the compounds below in Table 3b, and pharmaceutically acceptable salts and/or solvates thereof.

The present invention therefore provides the compounds below in Table 3c, and pharmaceutically acceptable salts and/or solvates thereof. The present invention therefore also provides stereoisomers of the compounds below in Table 3c, and pharmaceutically acceptable salts and/or solvates thereof.

The present invention therefore provides the compounds below in Table 3d, and pharmaceutically acceptable salts and/or solvates thereof. The present invention therefore also provides stereoisomers of the compounds below in Table 3d, and pharmaceutically acceptable salts and/or solvates thereof.

The present invention therefore provides the compounds below in Table 4, and pharmaceutically acceptable salts and/or solvates thereof. The present invention therefore also provides stereoisomers of the compounds below in Table 4, and pharmaceutically acceptable salts and/or solvates thereof.

TABLE 1a

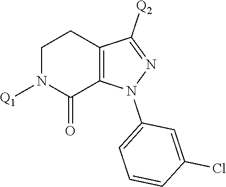

| Example | | |
|---|---|---|
| Number | Q1 | Q2 |
| 1001 | 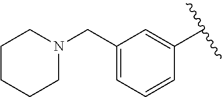 [SMILES: *c1cccc(CN2CCCCC2)c1] | 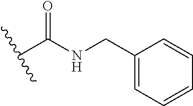 [SMILES: *C(=O)NCc1ccccc1] |
| 1002 | 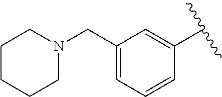 [SMILES: *c1cccc(CN2CCCCC2)c1] | 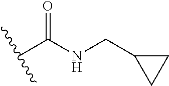 [SMILES: *C(=O)NCC1CC1] |
| 1003 | 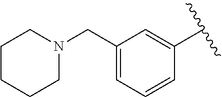 [SMILES: *c1cccc(CN2CCCCC2)c1] | 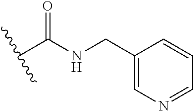 [SMILES: *C(=O)NCc1cccnc1] |
| 1004 | 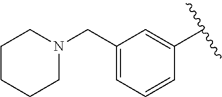 [SMILES: *c1cccc(CN2CCCCC2)c1] | 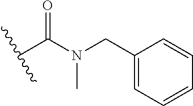 [SMILES: *C(=O)N(C)Cc1ccccc1] |
| 1005 | 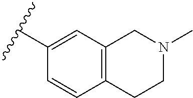 [SMILES: *c1ccc2c(c1)CN(C)CC2] | 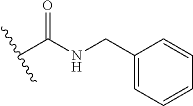 [SMILES: *C(=O)NCc1ccccc1] |
| 1006 | 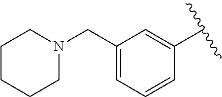 [SMILES: *c1cccc(CN2CCCCC2)c1] | 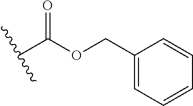 [SMILES: *C(=O)OCc1ccccc1] |
| 1007 | 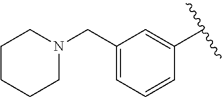 [SMILES: *c1cccc(CN2CCCCC2)c1] | 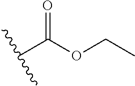 [SMILES: *C(=O)OCC] |
| 1008 | 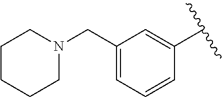 [SMILES: *c1cccc(CN2CCCCC2)c1] | 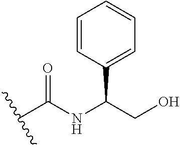 [SMILES: *C(=O)N[C@H](CO)c1ccccc1] |
| 1009 | 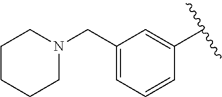 [SMILES: *c1cccc(CN2CCCCC2)c1] | 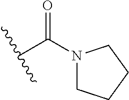 [SMILES: *C(=O)N1CCCC1] |
| 1010 | 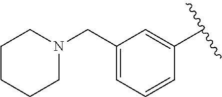 [SMILES: *c1cccc(CN2CCCCC2)c1] | 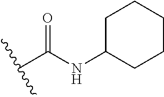 [SMILES: *C(=O)NC1CCCCC1] |
| 1011 | 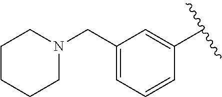 [SMILES: *c1cccc(CN2CCCCC2)c1] | 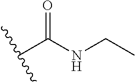 [SMILES: *C(=O)NCC] |
| 1012 | 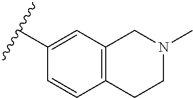 [SMILES: *c1ccc2c(c1)CN(C)CC2] | 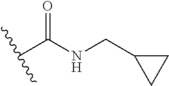 [SMILES: *C(=O)NCC1CC1] |
| 1013 | 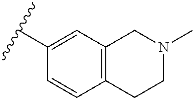 [SMILES: *c1ccc2c(c1)CN(C)CC2] | 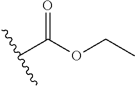 [SMILES: *C(=O)OCC] |
| 1014 | 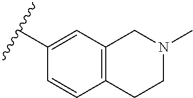 [SMILES: *c1ccc2c(c1)CN(C)CC2] | 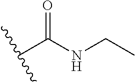 [SMILES: *C(=O)NCC] |
| 1015 | 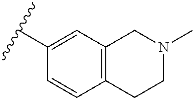 [SMILES: *c1ccc2c(c1)CN(C)CC2] | 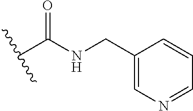 [SMILES: *C(=O)NCc1cccnc1] |
| 1016 | 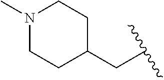 [SMILES: *CC1CCN(C)CC1] | 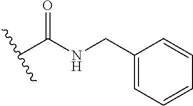 [SMILES: *C(=O)NCc1ccccc1] |
| 1017 | 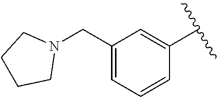 [SMILES: *c1cccc(CN2CCCC2)c1] | 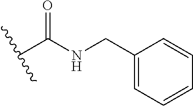 [SMILES: *C(=O)NCc1ccccc1] |
| 1018 | 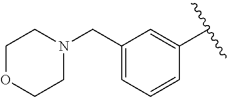 [SMILES: *c1cccc(CN2CCOCC2)c1] | 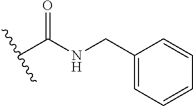 [SMILES: *C(=O)NCc1ccccc1] |
| 1019 | 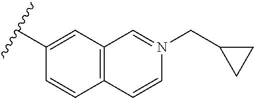 | 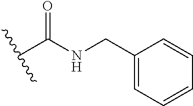 [SMILES: *C(=O)NCc1ccccc1] |
| 1020 | 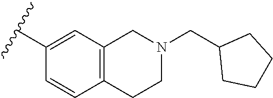 [SMILES: *c1ccc2c(c1)CN(CC1CCCC1)CC2] | 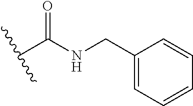 [SMILES: *C(=O)NCc1ccccc1] |
| 1021 | 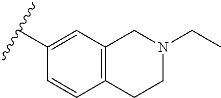 [SMILES: *c1ccc2c(c1)CN(CC)CC2] | 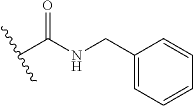 [SMILES: *C(=O)NCc1ccccc1] |
| 1022 | 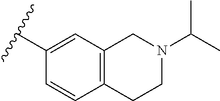 [SMILES: *c1ccc2c(c1)CN(C(C)C)CC2] | 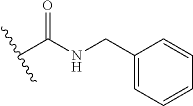 [SMILES: *C(=O)NCc1ccccc1] |
| 1023 | 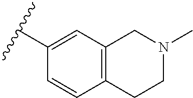 [SMILES: *c1ccc2c(c1)CN(C)CC2] | 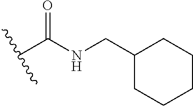 [SMILES: *C(=O)NCC1CCCCC1] |
| 1024 | 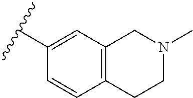 [SMILES: *c1ccc2c(c1)CN(C)CC2] | 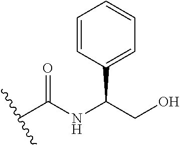 [SMILES: *C(=O)N[C@H](CO)c1ccccc1] |
| 1025 | 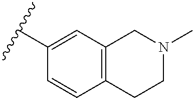 [SMILES: *c1ccc2c(c1)CN(C)CC2] | 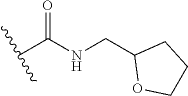 [SMILES: *C(=O)NCC1CCCO1] |
| 1026 | 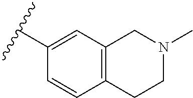 [SMILES: *c1ccc2c(c1)CN(C)CC2] | 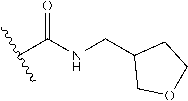 [SMILES: *C(=O)NCC1CCOC1] |
| 1027 | 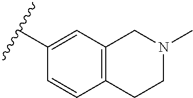 [SMILES: *c1ccc2c(c1)CN(C)CC2] | 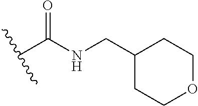 [SMILES: *C(=O)NCC1CCOCC1] |
| 1028 | 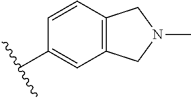 [SMILES: *c1ccc2c(c1)CN(C)C2] | 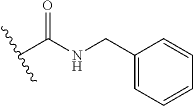 [SMILES: *C(=O)NCc1ccccc1] |
| 1029 | 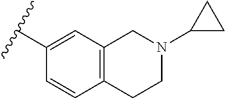 [SMILES: *c1ccc2c(c1)CN(C1CC1)CC2] | 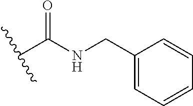 [SMILES: *C(=O)NCc1ccccc1] |
| 1030 | 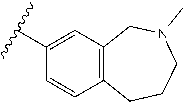 [SMILES: *c1ccc2c(c1)CN(C)CCC2] | 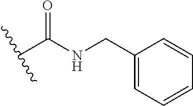 [SMILES: *C(=O)NCc1ccccc1] |
| 1031 | 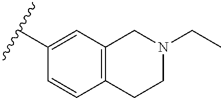 [SMILES: *c1ccc2c(c1)CN(CC)CC2] | 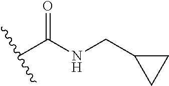 [SMILES: *C(=O)NCC1CC1] |
| 1032 | 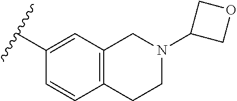 [SMILES: *c1ccc2c(c1)CN(C1COC1)CC2] | 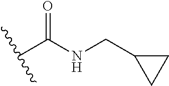 [SMILES: *C(=O)NCC1CC1] |
| 1033 | 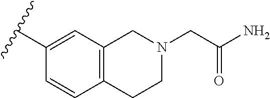 [SMILES: *c1ccc2c(c1)CN(CC(N)=O)CC2] | 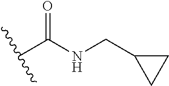 [SMILES: *C(=O)NCC1CC1] |
| 1034 | 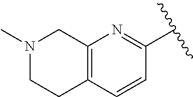 [SMILES: *c1ccc2c(n1)CN(C)CC2] | 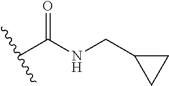 [SMILES: *C(=O)NCC1CC1] |
| 1035 | 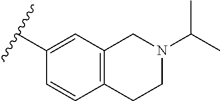 [SMILES: *c1ccc2c(c1)CN(C(C)C)CC2] | 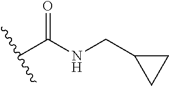 [SMILES: *C(=O)NCC1CC1] |
| 1036 | 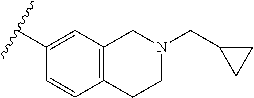 [SMILES: *c1ccc2c(c1)CN(CC1CC1)CC2] | 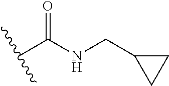 [SMILES: *C(=O)NCC1CC1] |
| 1037 | 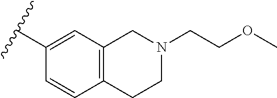 [SMILES: *c1ccc2c(c1)CN(CCOC)CC2] | 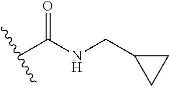 [SMILES: *C(=O)NCC1CC1] |
| 1038 | 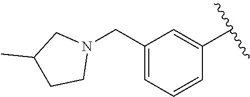 [SMILES: *c1cccc(CN2CCC(C)C2)c1] | 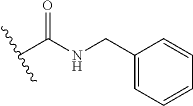 [SMILES: *C(=O)NCc1ccccc1] |
| 1039 | 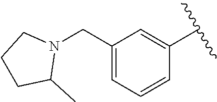 [SMILES: *c1cccc(CN2CCCC2C)c1] | 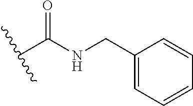 [SMILES: *C(=O)NCc1ccccc1] |
| 1040 | 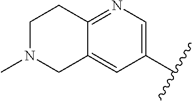 [SMILES: *c1cnc2c(c1)CN(C)CC2] | 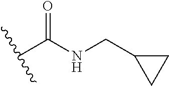 [SMILES: *C(=O)NCC1CC1] |
| 1041 | 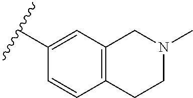 [SMILES: *c1ccc2c(c1)CN(C)CC2] | 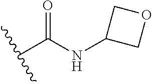 [SMILES: *C(=O)NC1COC1] |
| 1042 | 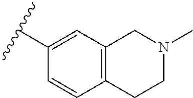 [SMILES: *c1ccc2c(c1)CN(C)CC2] | 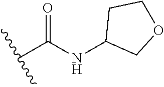 [SMILES: *C(=O)NC1CCOC1] |
| 1043 | 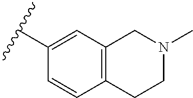 [SMILES: *c1ccc2c(c1)CN(C)CC2] | 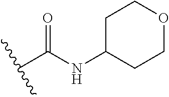 [SMILES: *C(=O)NC1CCOCC1] |
| 1044 | 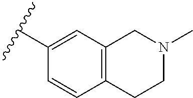 [SMILES: *c1ccc2c(c1)CN(C)CC2] | 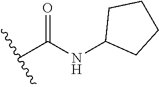 [SMILES: *C(=O)NC1CCCC1] |
| 1045 | 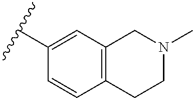 [SMILES: *c1ccc2c(c1)CN(C)CC2] | 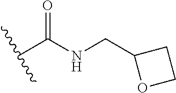 [SMILES: *C(=O)NCC1CCO1] |
| 1046 | 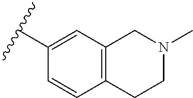 [SMILES: *c1ccc2c(c1)CN(C)CC2] | 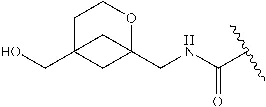 [SMILES: *C(=O)NCC12CC(CO)(CCO1)C2] |
| 1047 | 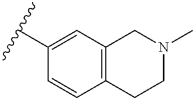 [SMILES: *c1ccc2c(c1)CN(C)CC2] | 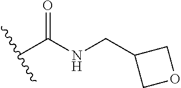 [SMILES: *C(=O)NCC1COC1] |
| 1048 | 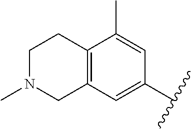 [SMILES: *c1cc(C)c2c(c1)CN(C)CC2] | 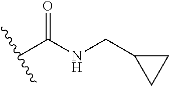 [SMILES: *C(=O)NCC1CC1] |
| 1049 | 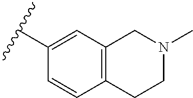 [SMILES: *c1ccc2c(c1)CN(C)CC2] | 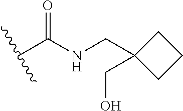 [SMILES: *C(=O)NCC1(CO)CCC1] |
| 1050 | 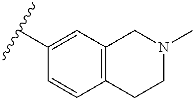 [SMILES: *c1ccc2c(c1)CN(C)CC2] | 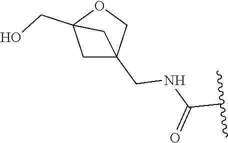 [SMILES: *C(=O)NCC12COC(CO)(C1)C2] |
| 1051 | 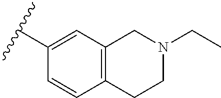 [SMILES: *c1ccc2c(c1)CN(CC)CC2] | 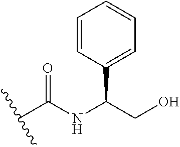 [SMILES: *C(=O)N[C@H](CO)c1ccccc1] |
| 1052 | 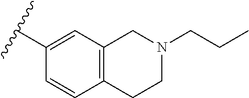 [SMILES: *c1ccc2c(c1)CN(CCC)CC2] | 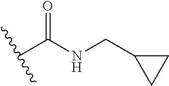 [SMILES: *C(=O)NCC1CC1] |
| 1053 | 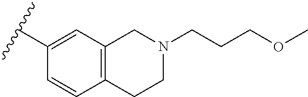 [SMILES: *c1ccc2c(c1)CN(CCCOC)CC2] | 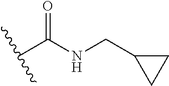 [SMILES: *C(=O)NCC1CC1] |
| 1054 | 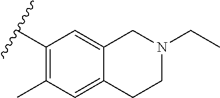 [SMILES: *c1cc2c(cc1C)CCN(CC)C2] | 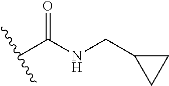 [SMILES: *C(=O)NCC1CC1] |
| 1055 | 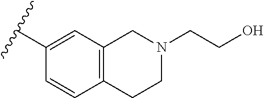 [SMILES: *c1ccc2c(c1)CN(CCO)CC2] | 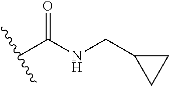 [SMILES: *C(=O)NCC1CC1] |
| 1056 | 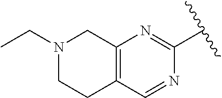 [SMILES: *c1ncc2c(n1)CN(CC)CC2] | 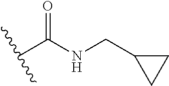 [SMILES: *C(=O)NCC1CC1] |
| 1057 | 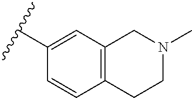 [SMILES: *c1ccc2c(c1)CN(C)CC2] | 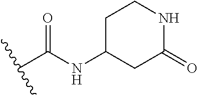 [SMILES: *C(=O)NC1CCNC(=O)C1] |
| 1058 | 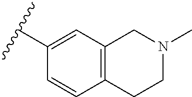 [SMILES: *c1ccc2c(c1)CN(C)CC2] | 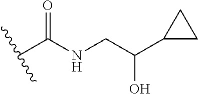 [SMILES: *C(=O)NCC(O)C1CC1] |
| 1059 | 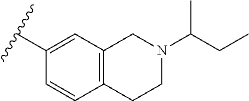 [SMILES: *c1ccc2c(c1)CN(C(C)CC)CC2] | 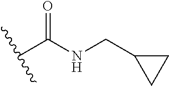 [SMILES: *C(=O)NCC1CC1] |
| 1060 | 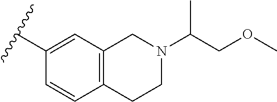 [SMILES: *c1ccc2c(c1)CN(C(C)COC)CC2] | 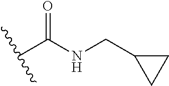 [SMILES: *C(=O)NCC1CC1] |
| 1061 | 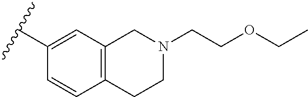 [SMILES: *c1ccc2c(c1)CN(CCOCC)CC2] | 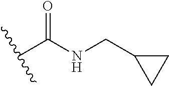 [SMILES: *C(=O)NCC1CC1] |
| 1063 | 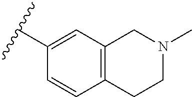 [SMILES: *c1ccc2c(c1)CN(C)CC2] | 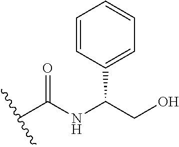 [SMILES: *C(=O)N[C@@H](CO)c1ccccc1] |
| 1064 | 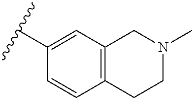 [SMILES: *c1ccc2c(c1)CN(C)CC2] | 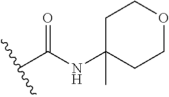 [SMILES: *C(=O)NC1(C)CCOCC1] |
| 1065 | 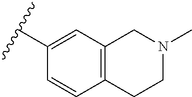 [SMILES: *c1ccc2c(c1)CN(C)CC2] | 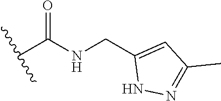 [SMILES: *C(=O)NCc1cc(C)n[nH]1] |
| 1066 | 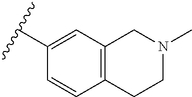 [SMILES: *c1ccc2c(c1)CN(C)CC2] | 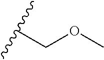 [SMILES: *COC] |
| 1067 | 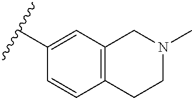 [SMILES: *c1ccc2c(c1)CN(C)CC2] | 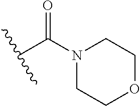 [SMILES: *C(=O)N1CCOCC1] |
| 1068 | 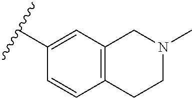 [SMILES: *c1ccc2c(c1)CN(C)CC2] | 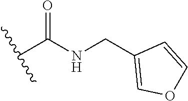 [SMILES: *C(=O)NCc1ccoc1] |
| 1069 | 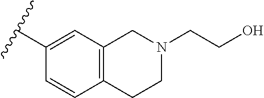 [SMILES: *c1ccc2c(c1)CN(CCO)CC2] | 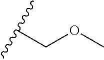 [SMILES: *COC] |
| 1070 | 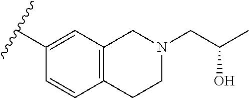 [SMILES: *c1ccc2c(c1)CN(C[C@H](C)O)CC2] | 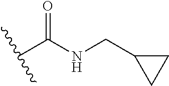 [SMILES: *C(=O)NCC1CC1] |
| 1071 | 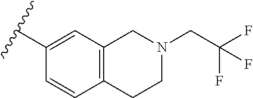 [SMILES: *c1ccc2c(c1)CN(CC(F)(F)F)CC2] | 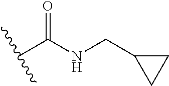 [SMILES: *C(=O)NCC1CC1] |
| 1072 | 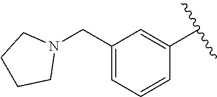 [SMILES: *c1cccc(CN2CCCC2)c1] | 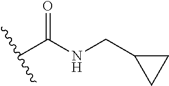 [SMILES: *C(=O)NCC1CC1] |
| 1073 | 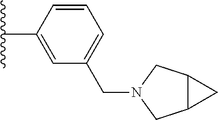 [SMILES: *c1cccc(CN2CC3CC3C2)c1] | 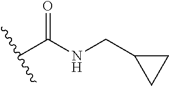 [SMILES: *C(=O)NCC1CC1] |
| 1074 | 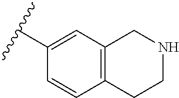 [SMILES: *c1ccc2c(c1)CNCC2] | 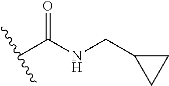 [SMILES: *C(=O)NCC1CC1] |
| 1075 | 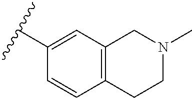 [SMILES: *c1ccc2c(c1)CN(C)CC2] | 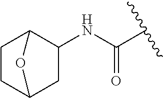 [SMILES: *C(=O)NC1CC2CCC1O2] |
| 1076 | 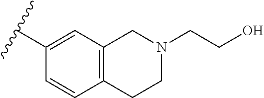 [SMILES: *c1ccc2c(c1)CN(CCO)CC2] | 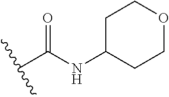 [SMILES: *C(=O)NC1CCOCC1] |
| 1077 | 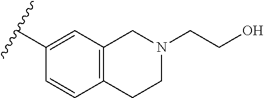 [SMILES: *c1ccc2c(c1)CN(CCO)CC2] | 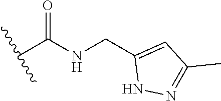 [SMILES: *C(=O)NCc1cc(C)n[nH]1] |
| 1078 | 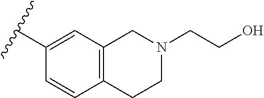 [SMILES: *c1ccc2c(c1)CN(CCO)CC2] | 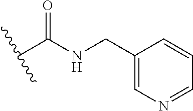 [SMILES: *C(=O)NCc1cccnc1] |
| 1079 | 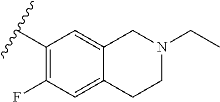 [SMILES: *c1cc2c(cc1F)CCN(CC)C2] | 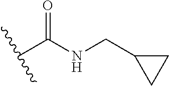 [SMILES: *C(=O)NCC1CC1] |
| 1080 | 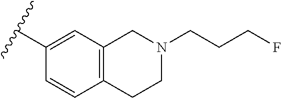 [SMILES: *c1ccc2c(c1)CN(CCCF)CC2] | 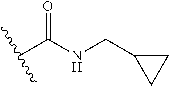 [SMILES: *C(=O)NCC1CC1] |
| 1081 | 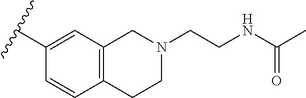 [SMILES: *c1ccc2c(c1)CN(CCNC(C)=O)CC2] | 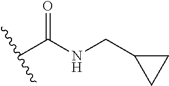 [SMILES: *C(=O)NCC1CC1] |
| 1082 | 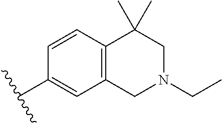 [SMILES: *c1ccc2c(c1)CN(CC)CC2(C)C] | 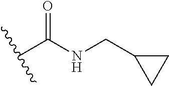 [SMILES: *C(=O)NCC1CC1] |
| 1083 | 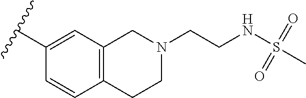 [SMILES: *c1ccc2c(c1)CN(CCNS(C)(=O)=O)CC2] | 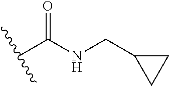 [SMILES: *C(=O)NCC1CC1] |
| 1084 | 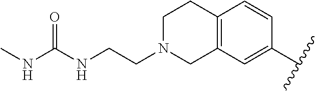 [SMILES: *c1ccc2c(c1)CN(CCNC(=O)NC)CC2] | 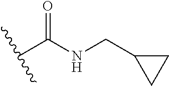 [SMILES: *C(=O)NCC1CC1] |
| 1085 | 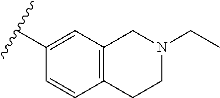 [SMILES: *c1ccc2c(c1)CN(CC)CC2] | 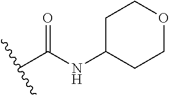 [SMILES: *C(=O)NC1CCOCC1] |
| 1086 | 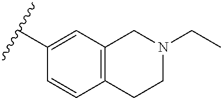 [SMILES: *c1ccc2c(c1)CN(CC)CC2] | 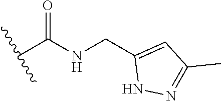 [SMILES: *C(=O)NCc1cc(C)n[nH]1] |
| 1087 | 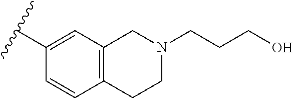 [SMILES: *c1ccc2c(c1)CN(CCCO)CC2] | 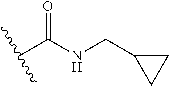 [SMILES: *C(=O)NCC1CC1] |
| 1088 | 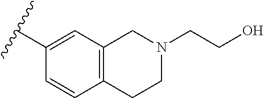 [SMILES: *c1ccc2c(c1)CN(CCO)CC2] | 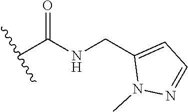 [SMILES: *C(=O)NCc1ccnn1C] |
| 1089 | 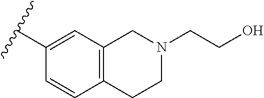 [SMILES: *c1ccc2c(c1)CN(CCO)CC2] | 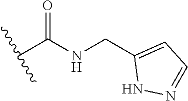 [SMILES: *C(=O)NCc1ccn[nH]1] |
| 1090 | 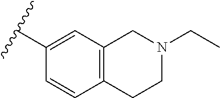 [SMILES: *c1ccc2c(c1)CN(CC)CC2] | 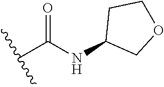 [SMILES: *C(=O)N[C@H]1CCOC1] |
| 1091 | 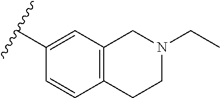 [SMILES: *c1ccc2c(c1)CN(CC)CC2] | 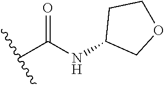 [SMILES: *C(=O)N[C@@H]1CCOC1] |
| 1092 | 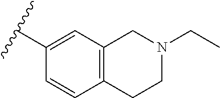 [SMILES: *c1ccc2c(c1)CN(CC)CC2] | 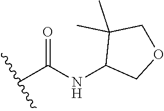 [SMILES: *C(=O)NC1COCC1(C)C] |
| 1093 | 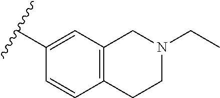 [SMILES: *c1ccc2c(c1)CN(CC)CC2] | 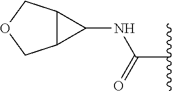 [SMILES: *C(=O)NC1C2COCC21] |
| 1094 | 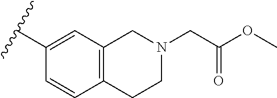 [SMILES: *c1ccc2c(c1)CN(CC(=O)OC)CC2] | 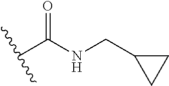 [SMILES: *C(=O)NCC1CC1] |
| 1095 | 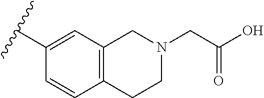 [SMILES: *c1ccc2c(c1)CN(CC(=O)O)CC2] | 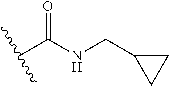 [SMILES: *C(=O)NCC1CC1] |
| 1096 | 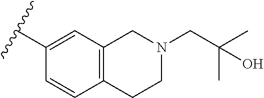 [SMILES: *c1ccc2c(c1)CN(CC(C)(C)O)CC2] | 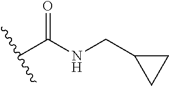 [SMILES: *C(=O)NCC1CC1] |
| 1097 | 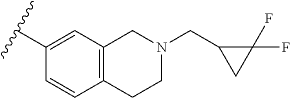 [SMILES: *c1ccc2c(c1)CN(CC1CC1(F)F)CC2] | 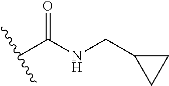 [SMILES: *C(=O)NCC1CC1] |
| 1098 | 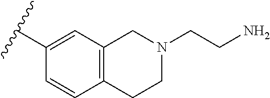 [SMILES: *c1ccc2c(c1)CN(CCN)CC2] | 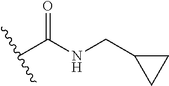 [SMILES: *C(=O)NCC1CC1] |
| 1099 | 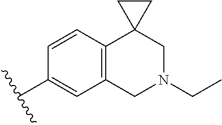 [SMILES: *c1ccc2c(c1)CN(CC)CC21CC1] | 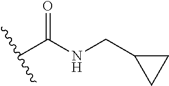 [SMILES: *C(=O)NCC1CC1] |
| 1100 | 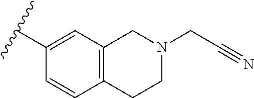 [SMILES: *c1ccc2c(c1)CN(CC#N)CC2] | 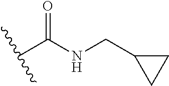 [SMILES: *C(=O)NCC1CC1] |
| 1101 | 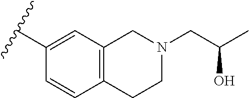 [SMILES: *c1ccc2c(c1)CN(C[C@@H](C)O)CC2] | 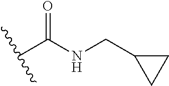 [SMILES: *C(=O)NCC1CC1] |
| 1102 | 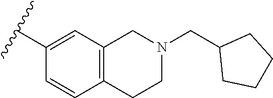 [SMILES: *c1ccc2c(c1)CN(CC1CCCC1)CC2] | 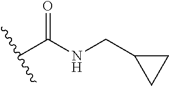 [SMILES: *C(=O)NCC1CC1] |
| 1103 | 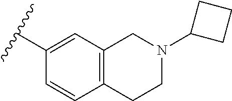 [SMILES: *c1ccc2c(c1)CN(C1CCC1)CC2] | 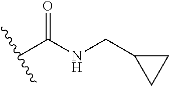 [SMILES: *C(=O)NCC1CC1] |
| 1104 | 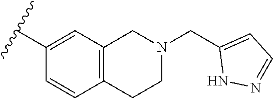 [SMILES: *c1ccc2c(c1)CN(Cc1ccn[nH]1)CC2] | 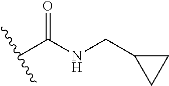 [SMILES: *C(=O)NCC1CC1] |
| 1105 | 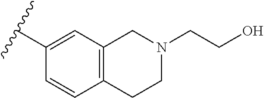 [SMILES: *c1ccc2c(c1)CN(CCO)CC2] | 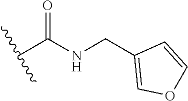 [SMILES: *C(=O)NCc1ccoc1] |
| 1106 | 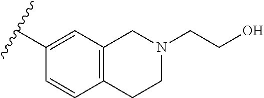 [SMILES: *c1ccc2c(c1)CN(CCO)CC2] | 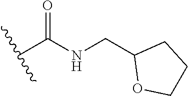 [SMILES: *C(=O)NCC1CCCO1] |
| 1107 | 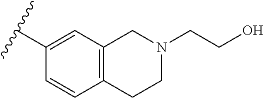 [SMILES: *c1ccc2c(c1)CN(CCO)CC2] | 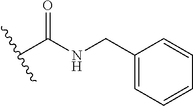 [SMILES: *C(=O)NCc1ccccc1] |
| 1108 | 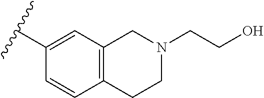 [SMILES: *c1ccc2c(c1)CN(CCO)CC2] | 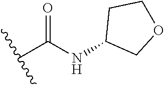 [SMILES: *C(=O)N[C@@H]1CCOC1] |
| 1109 | 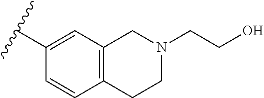 [SMILES: *c1ccc2c(c1)CN(CCO)CC2] | 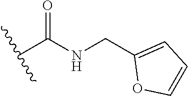 [SMILES: *C(=O)NCc1ccco1] |
| 1110 | 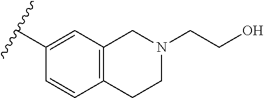 [SMILES: *c1ccc2c(c1)CN(CCO)CC2] | 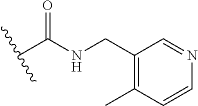 [SMILES: *C(=O)NCc1cnccc1C] |
| 1111 | 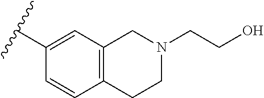 [SMILES: *c1ccc2c(c1)CN(CCO)CC2] | 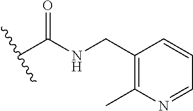 [SMILES: *C(=O)NCc1cccnc1C] |
| 1112 | 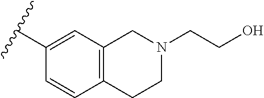 [SMILES: *c1ccc2c(c1)CN(CCO)CC2] | 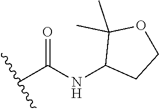 [SMILES: *C(=O)NC1CCOC1(C)C] |
| 1113 | 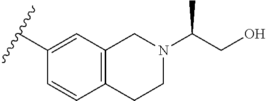 [SMILES: *c1ccc2c(c1)CN([C@@H](C)CO)CC2] | 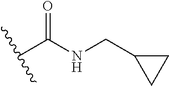 [SMILES: *C(=O)NCC1CC1] |
| 1114 | 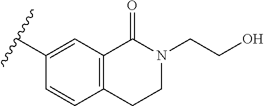 [SMILES: *c1ccc2c(c1)C(=O)N(CCO)CC2] | 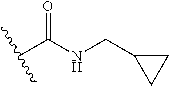 [SMILES: *C(=O)NCC1CC1] |
| 1115 | 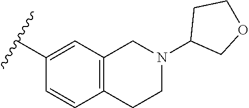 [SMILES: *c1ccc2c(c1)CN(C1CCOC1)CC2] | 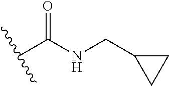 [SMILES: *C(=O)NCC1CC1] |
| 1116 | 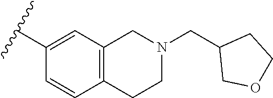 [SMILES: *c1ccc2c(c1)CN(CC1CCOC1)CC2] | 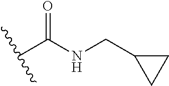 [SMILES: *C(=O)NCC1CC1] |
| 1117 | 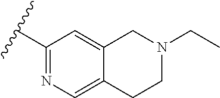 [SMILES: *c1cc2c(cn1)CCN(CC)C2] | 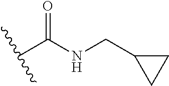 [SMILES: *C(=O)NCC1CC1] |
| 1118 | 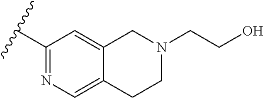 [SMILES: *c1cc2c(cn1)CCN(CCO)C2] | 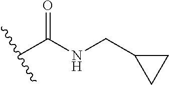 [SMILES: *C(=O)NCC1CC1] |
| 1119 | 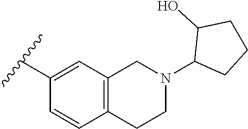 [SMILES: *c1ccc2c(c1)CN(C1CCCC1O)CC2] | 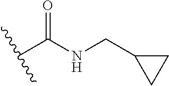 [SMILES: *C(=O)NCC1CC1] |
| 1120 | 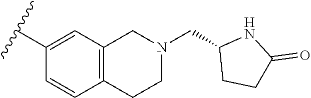 [SMILES: *c1ccc2c(c1)CN(C[C@H]1CCC(=O)N1)CC2] | 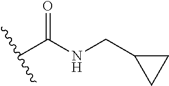 [SMILES: *C(=O)NCC1CC1] |
| 1121 | 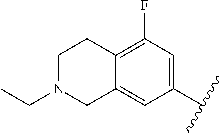 [SMILES: *c1cc(F)c2c(c1)CN(CC)CC2] | 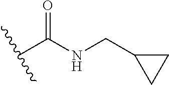 [SMILES: *C(=O)NCC1CC1] |
| 1122 | 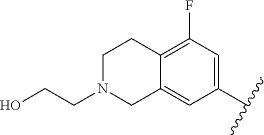 [SMILES: *c1cc(F)c2c(c1)CN(CCO)CC2] | 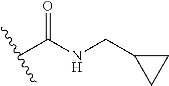 [SMILES: *C(=O)NCC1CC1] |
| 1123 | 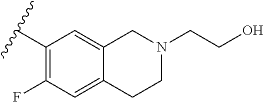 [SMILES: *c1cc2c(cc1F)CCN(CCO)C2] | 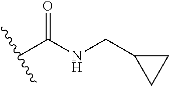 [SMILES: *C(=O)NCC1CC1] |
| 1124 | 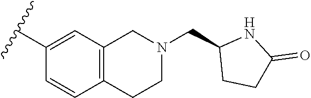 [SMILES: *c1ccc2c(c1)CN(C[C@@H]1CCC(=O)N1)CC2] | 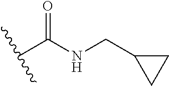 [SMILES: *C(=O)NCC1CC1] |
| 1125 | 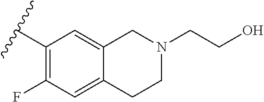 [SMILES: *c1cc2c(cc1F)CCN(CCO)C2] | 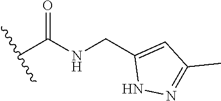 [SMILES: *C(=O)NCc1cc(C)n[nH]1] |
| 1126 | 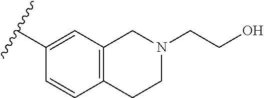 [SMILES: *c1ccc2c(c1)CN(CCO)CC2] | 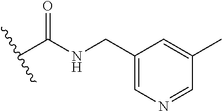 [SMILES: *C(=O)NCc1cncc(C)c1] |
| 1127 | 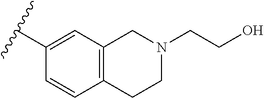 [SMILES: *c1ccc2c(c1)CN(CCO)CC2] | 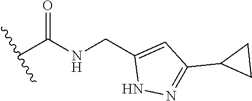 [SMILES: *C(=O)NCc1cc(C2CC2)n[nH]1] |
| 1128 | 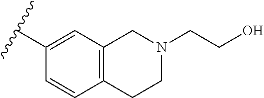 [SMILES: *c1ccc2c(c1)CN(CCO)CC2] | 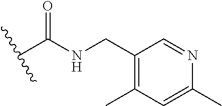 [SMILES: *C(=O)NCc1cnc(C)cc1C] |
| 1129 | 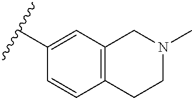 [SMILES: *c1ccc2c(c1)CN(C)CC2] | 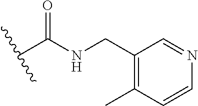 [SMILES: *C(=O)NCc1cnccc1C] |
| 1130 | 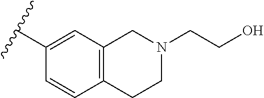 [SMILES: *c1ccc2c(c1)CN(CCO)CC2] | 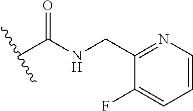 [SMILES: *C(=O)NCc1ncccc1F] |
| 1131 | 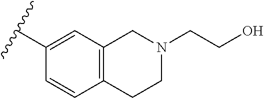 [SMILES: *c1ccc2c(c1)CN(CCO)CC2] | 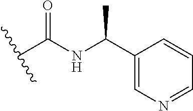 [SMILES: *C(=O)N[C@@H](C)c1cccnc1] |
| 1132 | 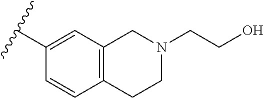 [SMILES: *c1ccc2c(c1)CN(CCO)CC2] | 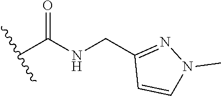 [SMILES: *C(=O)NCc1ccn(C)n1] |
| 1133 | 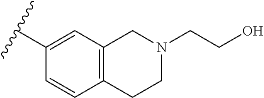 [SMILES: *c1ccc2c(c1)CN(CCO)CC2] | 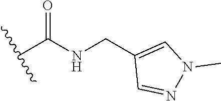 [SMILES: *C(=O)NCc1cnn(C)c1] |
| 1134 | 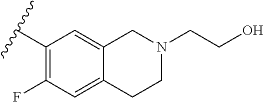 [SMILES: *c1cc2c(cc1F)CCN(CCO)C2] | 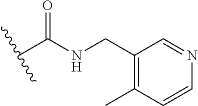 [SMILES: *C(=O)NCc1cnccc1C] |
| 1135 | 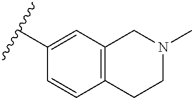 [SMILES: *c1ccc2c(c1)CN(C)CC2] | 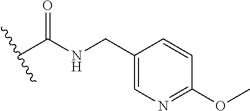 [SMILES: *C(=O)NCc1ccc(OC)nc1] |
| 1136 | 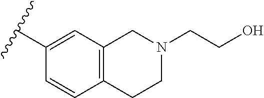 [SMILES: *c1ccc2c(c1)CN(CCO)CC2] | 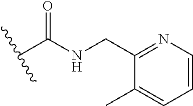 [SMILES: *C(=O)NCc1ncccc1C] |
| 1137 | 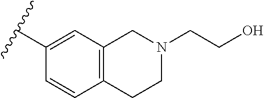 [SMILES: *c1ccc2c(c1)CN(CCO)CC2] | 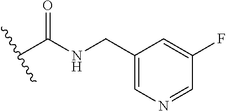 [SMILES: *C(=O)NCc1cncc(F)c1] |
| 1138 | 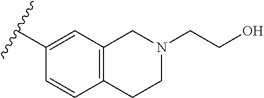 [SMILES: *c1ccc2c(c1)CN(CCO)CC2] | 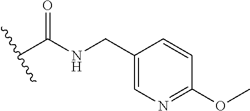 [SMILES: *C(=O)NCc1ccc(OC)nc1] |
| 1139 | 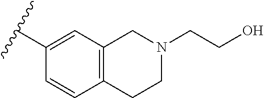 [SMILES: *c1ccc2c(c1)CN(CCO)CC2] | 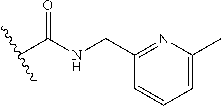 [SMILES: *C(=O)NCc1cccc(C)n1] |
| 1140 | 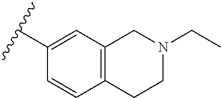 [SMILES: *c1ccc2c(c1)CN(CC)CC2] | 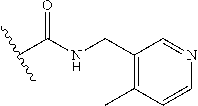 [SMILES: *C(=O)NCc1cnccc1C] |
| 1141 | 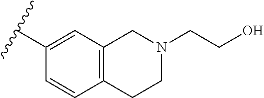 [SMILES: *c1ccc2c(c1)CN(CCO)CC2] | 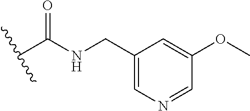 [SMILES: *C(=O)NCc1cncc(OC)c1] |
| 1142 | 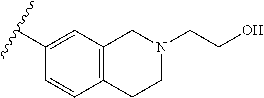 [SMILES: *c1ccc2c(c1)CN(CCO)CC2] | 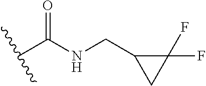 [SMILES: *C(=O)NCC1CC1(F)F] |
| 1143 | 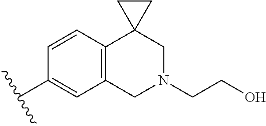 [SMILES: *c1ccc2c(c1)CN(CCO)CC21CC1] | 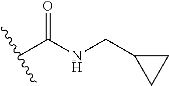 [SMILES: *C(=O)NCC1CC1] |
| 1144 | 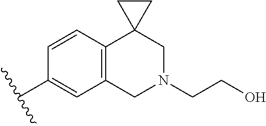 [SMILES: *c1ccc2c(c1)CN(CCO)CC21CC1] | 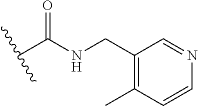 [SMILES: *C(=O)NCc1cnccc1C] |
| 1145 | 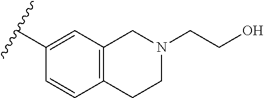 [SMILES: *c1ccc2c(c1)CN(CCO)CC2] | 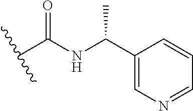 [SMILES: *C(=O)N[C@H](C)c1cccnc1] |
| 1146 | 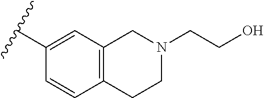 [SMILES: *c1ccc2c(c1)CN(CCO)CC2] | 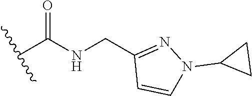 [SMILES: *C(=O)NCc1ccn(C2CC2)n1] |
| 1147 | 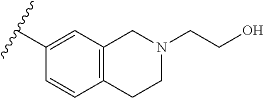 [SMILES: *c1ccc2c(c1)CN(CCO)CC2] | 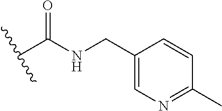 [SMILES: *C(=O)NCc1ccc(C)nc1] |
| 1148 | 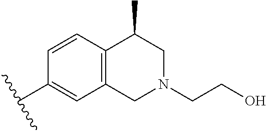 [SMILES: *c1ccc2c(c1)CN(CCO)C[C@@H]2C] | 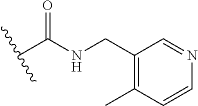 [SMILES: *C(=O)NCc1cnccc1C] |
| 1149 | 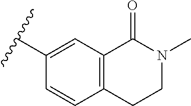 [SMILES: *c1ccc2c(c1)C(=O)N(C)CC2] | 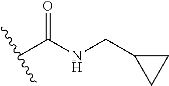 [SMILES: *C(=O)NCC1CC1] |
| 1150 | 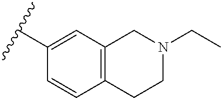 [SMILES: *c1ccc2c(c1)CN(CC)CC2] | 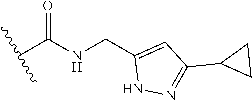 [SMILES: *C(=O)NCc1cc(C2CC2)n[nH]1] |
| 1151 | 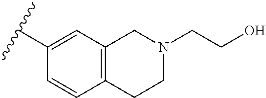 [SMILES: *c1ccc2c(c1)CN(CCO)CC2] | 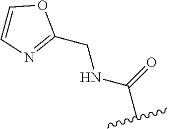 [SMILES: *C(=O)NCc1ncco1] |
| 1152 | 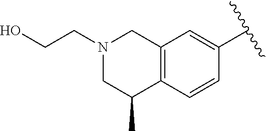 [SMILES: *c1ccc2c(c1)CN(CCO)C[C@@H]2C] | 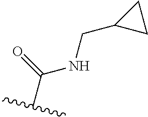 [SMILES: *C(=O)NCC1CC1] |
| 1153 | 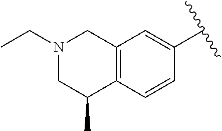 [SMILES: *c1ccc2c(c1)CN(CC)C[C@@H]2C] | 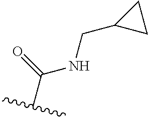 [SMILES: *C(=O)NCC1CC1] |
| 1154 | 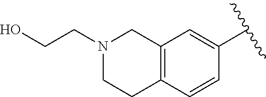 [SMILES: *c1ccc2c(c1)CN(CCO)CC2] | 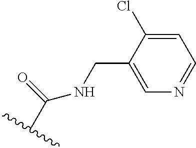 [SMILES: *C(=O)NCc1cnccc1Cl] |
| 1155 | 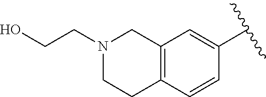 [SMILES: *c1ccc2c(c1)CN(CCO)CC2] | 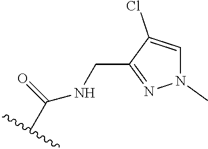 [SMILES: *C(=O)NCc1nn(C)cc1Cl] |
| 1156 | 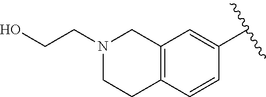 [SMILES: *c1ccc2c(c1)CN(CCO)CC2] | 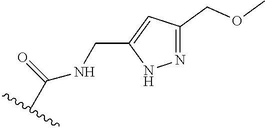 [SMILES: *C(=O)NCc1cc(COC)n[nH]1] |
| 1157 | 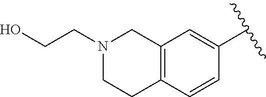 [SMILES: *c1ccc2c(c1)CN(CCO)CC2] | 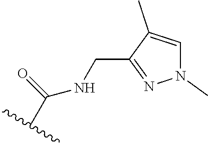 [SMILES: *C(=O)NCc1nn(C)cc1C] |
| 1158 | 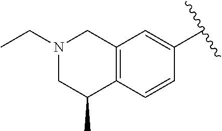 [SMILES: *c1ccc2c(c1)CN(CC)C[C@@H]2C] | 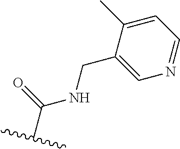 [SMILES: *C(=O)NCc1cnccc1C] |
| 1159 | 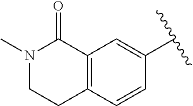 [SMILES: *c1ccc2c(c1)C(=O)N(C)CC2] | 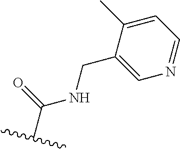 [SMILES: *C(=O)NCc1cnccc1C] |
| 1160 | 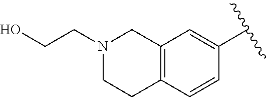 [SMILES: *c1ccc2c(c1)CN(CCO)CC2] | 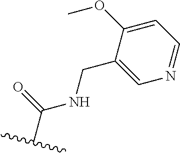 [SMILES: *C(=O)NCc1cnccc1OC] |
| 1161 | 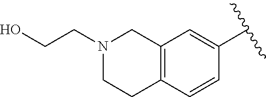 [SMILES: *c1ccc2c(c1)CN(CCO)CC2] | 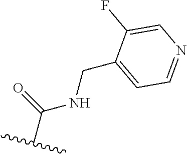 [SMILES: *C(=O)NCc1ccncc1F] |
| 1162 | 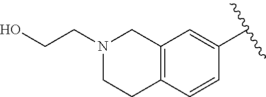 [SMILES: *c1ccc2c(c1)CN(CCO)CC2] | 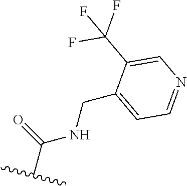 [SMILES: *C(=O)NCc1ccncc1C(F)(F)F] |
| 1163 | 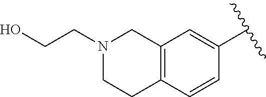 [SMILES: *c1ccc2c(c1)CN(CCO)CC2] | 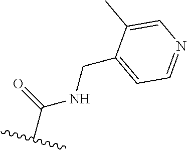 [SMILES: *C(=O)NCc1ccncc1C] |
| 1164 | 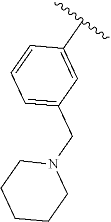 [SMILES: *c1cccc(CN2CCCCC2)c1] | 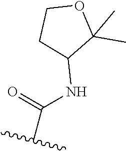 [SMILES: *C(=O)NC1CCOC1(C)C] |
| 1165 | 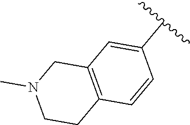 [SMILES: *c1ccc2c(c1)CN(C)CC2] | 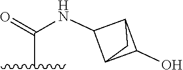 [SMILES: *C(=O)NC1C2CC1C2O] |
| 1166 | 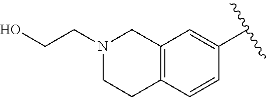 [SMILES: *c1ccc2c(c1)CN(CCO)CC2] | 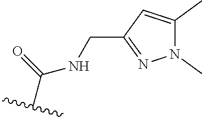 [SMILES: *C(=O)NCc1cc(C)n(C)n1] |
| 1167 | 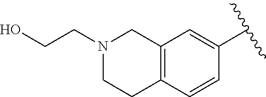 [SMILES: *c1ccc2c(c1)CN(CCO)CC2] | 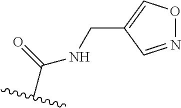 [SMILES: *C(=O)NCc1cnoc1] |
| 1168 | 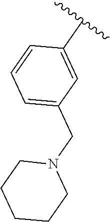 [SMILES: *c1cccc(CN2CCCCC2)c1] | 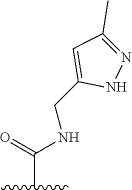 [SMILES: *C(=O)NCc1cc(C)n[nH]1] |
| 1169 | 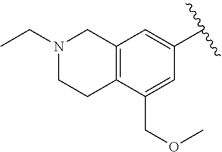 [SMILES: *c1cc(COC)c2c(c1)CN(CC)CC2] | 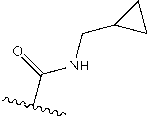 [SMILES: *C(=O)NCC1CC1] |
| 1170 | 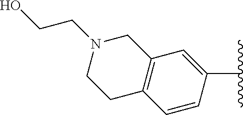 [SMILES: *c1ccc2c(c1)CN(CCO)CC2] | 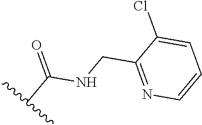 [SMILES: *C(=O)NCc1ncccc1Cl] |
| 1171 | 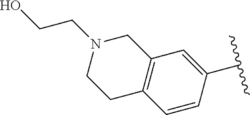 [SMILES: *c1ccc2c(c1)CN(CCO)CC2] | 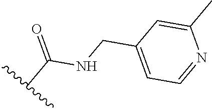 [SMILES: *C(=O)NCc1ccnc(C)c1] |
| 1172 | 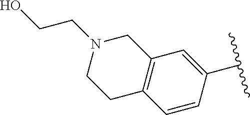 [SMILES: *c1ccc2c(c1)CN(CCO)CC2] | 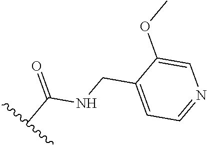 [SMILES: *C(=O)NCc1ccncc1OC] |
| 1173 | 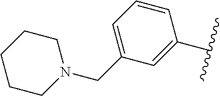 [SMILES: *c1cccc(CN2CCCCC2)c1] | 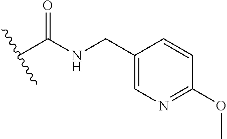 [SMILES: *C(=O)NCc1ccc(OC)nc1] |
| 1174 | 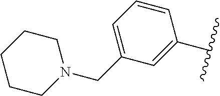 [SMILES: *c1cccc(CN2CCCCC2)c1] | 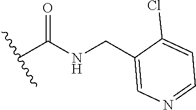 [SMILES: *C(=O)NCc1cnccc1Cl] |
| 1175 | 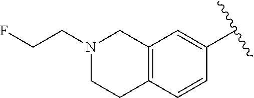 [SMILES: *c1ccc2c(c1)CN(CCF)CC2] | 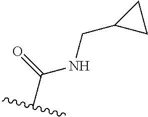 [SMILES: *C(=O)NCC1CC1] |
| 1176 | 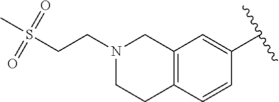 [SMILES: *c1ccc2c(c1)CN(CCS(C)(=O)=O)CC2] | 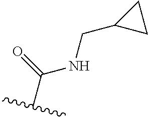 [SMILES: *C(=O)NCC1CC1] |
| 1177 | 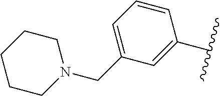 [SMILES: *c1cccc(CN2CCCCC2)c1] | 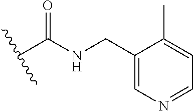 [SMILES: *C(=O)NCc1cnccc1C] |
| 1178 | 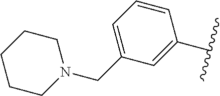 [SMILES: *c1cccc(CN2CCCCC2)c1] | 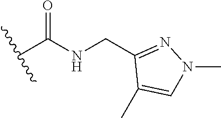 [SMILES: *C(=O)NCc1nn(C)cc1C] |

TABLE 1b

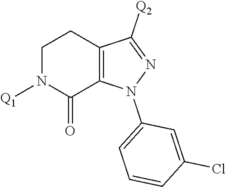

| Example | | |
|---|---|---|
| Number | Q1 | Q2 |
| 1179 | 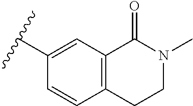 [SMILES: *c1ccc2c(c1)C(=O)N(C)CC2] | 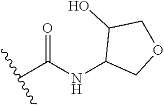 [SMILES: *C(=O)NC1COCC1O] |
| 1180 | 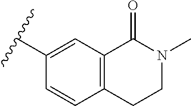 [SMILES: *c1ccc2c(c1)C(=O)N(C)CC2] | 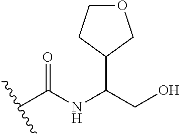 [SMILES: *C(=O)NC(CO)C1CCOC1] |
| 1181 | 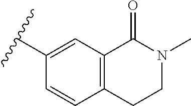 [SMILES: *c1ccc2c(c1)C(=O)N(C)CC2] | 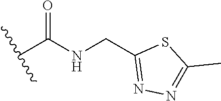 [SMILES: *C(=O)NCc1nnc(C)s1] |
| 1182 | 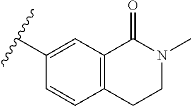 [SMILES: *c1ccc2c(c1)C(=O)N(C)CC2] | 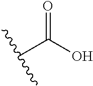 [SMILES: *C(=O)O] |
| 1183 | 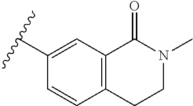 [SMILES: *c1ccc2c(c1)C(=O)N(C)CC2] | 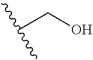 [SMILES: *CO] |
| 1184 | 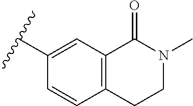 [SMILES: *c1ccc2c(c1)C(=O)N(C)CC2] | 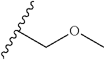 [SMILES: *COC] |
| 1185 | 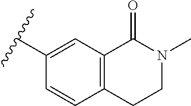 [SMILES: *c1ccc2c(c1)C(=O)N(C)CC2] | 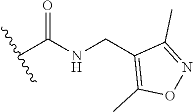 [SMILES: *C(=O)NCc1c(C)noc1C] |
| 1186 | 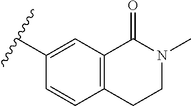 [SMILES: *c1ccc2c(c1)C(=O)N(C)CC2] | 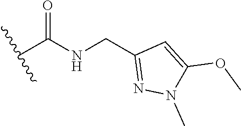 [SMILES: *C(=O)NCc1cc(OC)n(C)n1] |
| 1187 | 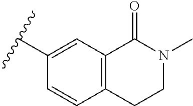 [SMILES: *c1ccc2c(c1)C(=O)N(C)CC2] | 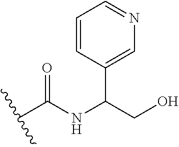 [SMILES: *C(=O)NC(CO)c1cccnc1] |
| 1188 | 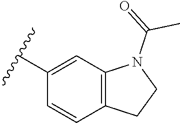 [SMILES: *c1ccc2c(c1)N(C(C)=O)CC2] | 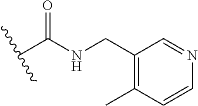 [SMILES: *C(=O)NCc1cnccc1C] |
| 1189 | 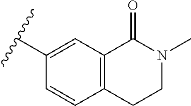 [SMILES: *c1ccc2c(c1)C(=O)N(C)CC2] | 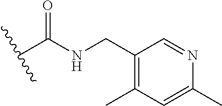 [SMILES: *C(=O)NCc1cnc(C)cc1C] |
| 1190 | 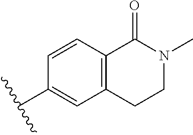 [SMILES: *c1ccc2c(c1)CCN(C)C2=O] | 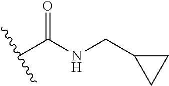 [SMILES: *C(=O)NCC1CC1] |
| 1191 | 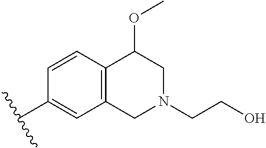 [SMILES: *c1ccc2c(c1)CN(CCO)CC2OC] | 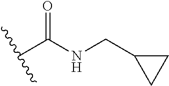 [SMILES: *C(=O)NCC1CC1] |
| 1192 | 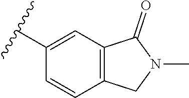 [SMILES: *c1ccc2c(c1)C(=O)N(C)C2] | 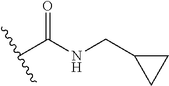 [SMILES: *C(=O)NCC1CC1] |
| 1193 | 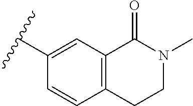 [SMILES: *c1ccc2c(c1)C(=O)N(C)CC2] | 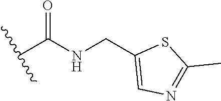 [SMILES: *C(=O)NCc1cnc(C)s1] |
| 1194 | 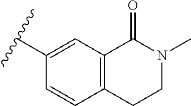 [SMILES: *c1ccc2c(c1)C(=O)N(C)CC2] | 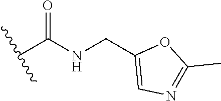 [SMILES: *C(=O)NCc1cnc(C)o1] |
| 1195 | 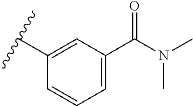 [SMILES: *c1cccc(C(=O)N(C)C)c1] | 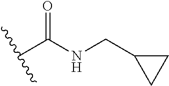 [SMILES: *C(=O)NCC1CC1] |
| 1196 | 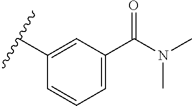 [SMILES: *c1cccc(C(=O)N(C)C)c1] | 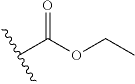 [SMILES: *C(=O)OCC] |
| 1197 | 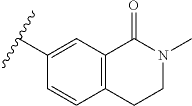 [SMILES: *c1ccc2c(c1)C(=O)N(C)CC2] | 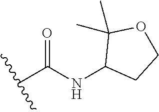 [SMILES: *C(=O)NC1CCOC1(C)C] |
| 1198 | 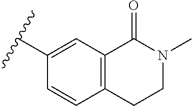 [SMILES: *c1ccc2c(c1)C(=O)N(C)CC2] | 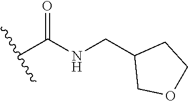 [SMILES: *C(=O)NCC1CCOC1] |
| 1199 | 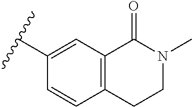 [SMILES: *c1ccc2c(c1)C(=O)N(C)CC2] | 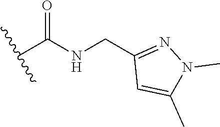 [SMILES: *C(=O)NCc1cc(C)n(C)n1] |
| 1200 | 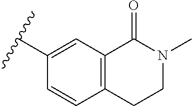 [SMILES: *c1ccc2c(c1)C(=O)N(C)CC2] | 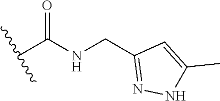 [SMILES: *C(=O)NCc1cc(C)[nH]n1] |
| 1201 | 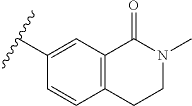 [SMILES: *c1ccc2c(c1)C(=O)N(C)CC2] | 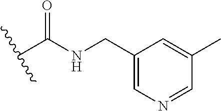 [SMILES: *C(=O)NCc1cncc(C)c1] |
| 1202 | 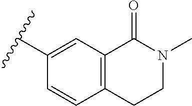 [SMILES: *c1ccc2c(c1)C(=O)N(C)CC2] | 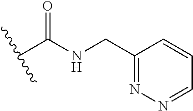 [SMILES: *C(=O)NCc1cccnn1] |
| 1203 | 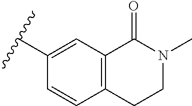 [SMILES: *c1ccc2c(c1)C(=O)N(C)CC2] | 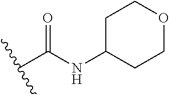 [SMILES: *C(=O)NC1CCOCC1] |
| 1204 | 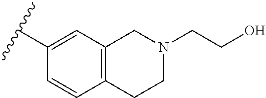 [SMILES: *c1ccc2c(c1)CN(CCO)CC2] | 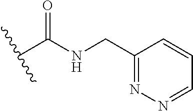 [SMILES: *C(=O)NCc1cccnn1] |
| 1205 | 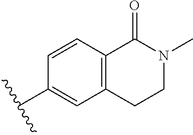 [SMILES: *c1ccc2c(c1)CCN(C)C2=O] | 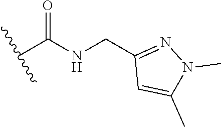 [SMILES: *C(=O)NCc1cc(C)n(C)n1] |
| 1206 | 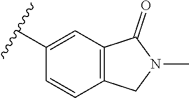 [SMILES: *c1ccc2c(c1)C(=O)N(C)C2] | 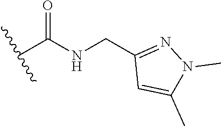 [SMILES: *C(=O)NCc1cc(C)n(C)n1] |
| 1207 | 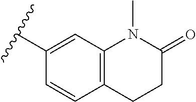 [SMILES: *c1ccc2c(c1)N(C)C(=O)CC2] | 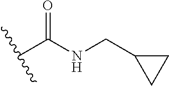 [SMILES: *C(=O)NCC1CC1] |
| 1208 | 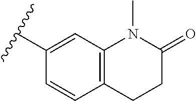 [SMILES: *c1ccc2c(c1)N(C)C(=O)CC2] | 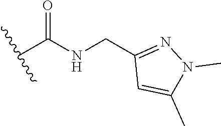 [SMILES: *C(=O)NCc1cc(C)n(C)n1] |
| 1209 | 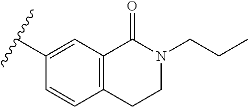 [SMILES: *c1ccc2c(c1)C(=O)N(CCC)CC2] | 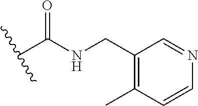 [SMILES: *C(=O)NCc1cnccc1C] |
| 1210 | 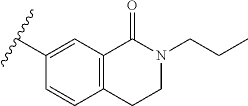 [SMILES: *c1ccc2c(c1)C(=O)N(CCC)CC2] | 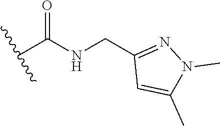 [SMILES: *C(=O)NCc1cc(C)n(C)n1] |
| 1211 | 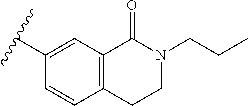 [SMILES: *c1ccc2c(c1)C(=O)N(CCC)CC2] | 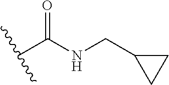 [SMILES: *C(=O)NCC1CC1] |
| 1212 | 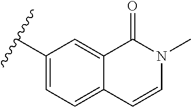 [SMILES: *c1ccc2ccn(C)c(=O)c2c1] | 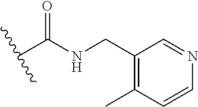 [SMILES: *C(=O)NCc1cnccc1C] |
| 1213 | 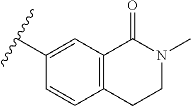 [SMILES: *c1ccc2c(c1)C(=O)N(C)CC2] | 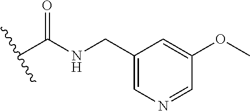 [SMILES: *C(=O)NCc1cncc(OC)c1] |
| 1214 | 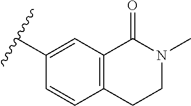 [SMILES: *c1ccc2c(c1)C(=O)N(C)CC2] | 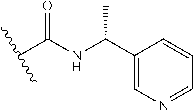 [SMILES: *C(=O)N[C@H](C)c1cccnc1] |
| 1215 | 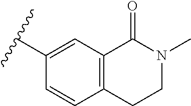 [SMILES: *c1ccc2c(c1)C(=O)N(C)CC2] | 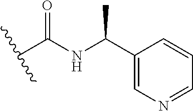 [SMILES: *C(=O)N[C@@H](C)c1cccnc1] |
| 1216 | 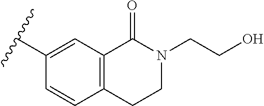 [SMILES: *c1ccc2c(c1)C(=O)N(CCO)CC2] | 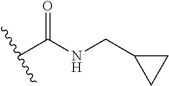 [SMILES: *C(=O)NCC1CC1] |
| 1217 | 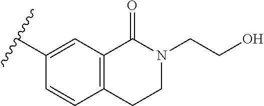 [SMILES: *c1ccc2c(c1)C(=O)N(CCO)CC2] | 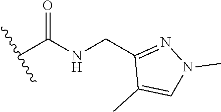 [SMILES: *C(=O)NCc1nn(C)cc1C] |
| 1218 | 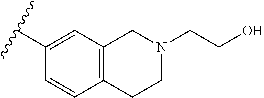 [SMILES: *c1ccc2c(c1)CN(CCO)CC2] | 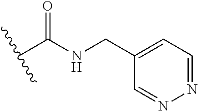 [SMILES: *C(=O)NCc1ccnnc1] |
| 1219 | 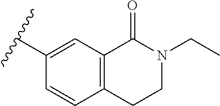 [SMILES: *c1ccc2c(c1)C(=O)N(CC)CC2] | 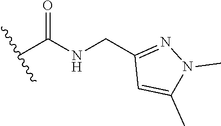 [SMILES: *C(=O)NCc1cc(C)n(C)n1] |
| 1220 | 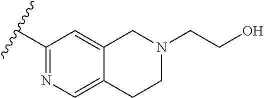 [SMILES: *c1cc2c(cn1)CCN(CCO)C2] | 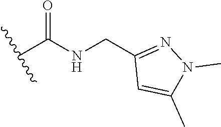 [SMILES: *C(=O)NCc1cc(C)n(C)n1] |
| 1221 | 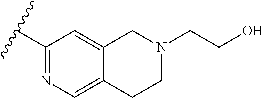 [SMILES: *c1cc2c(cn1)CCN(CCO)C2] | 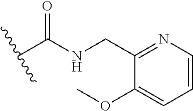 [SMILES: *C(=O)NCc1ncccc1OC] |
| 1222 | 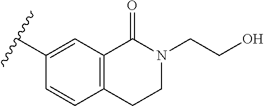 [SMILES: *c1ccc2c(c1)C(=O)N(CCO)CC2] | 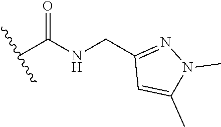 [SMILES: *C(=O)NCc1cc(C)n(C)n1] |
| 1223 | 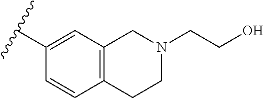 [SMILES: *c1ccc2c(c1)CN(CCO)CC2] | 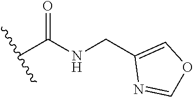 [SMILES: *C(=O)NCc1cocn1] |
| 1224 | 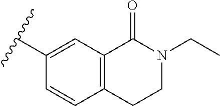 [SMILES: *c1ccc2c(c1)C(=O)N(CC)CC2] | 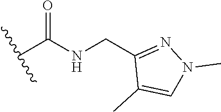 [SMILES: *C(=O)NCc1nn(C)cc1C] |
| 1225 | 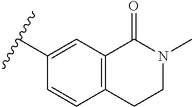 [SMILES: *c1ccc2c(c1)C(=O)N(C)CC2] | 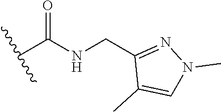 [SMILES: *C(=O)NCc1nn(C)cc1C] |
| 1226 | 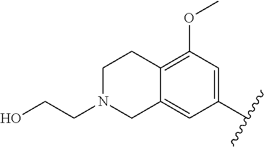 [SMILES: *c1cc2c(c(OC)c1)CCN(CCO)C2] | 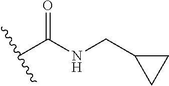 [SMILES: *C(=O)NCC1CC1] |
| 1227 | 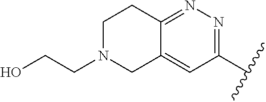 [SMILES: *c1cc2c(nn1)CCN(CCO)C2] | 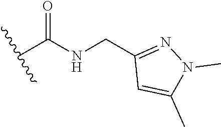 [SMILES: *C(=O)NCc1cc(C)n(C)n1] |
| 1229 | 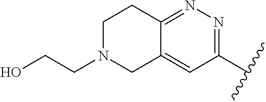 [SMILES: *c1cc2c(nn1)CCN(CCO)C2] | 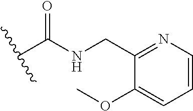 [SMILES: *C(=O)NCc1ncccc1OC] |
| 1230 | 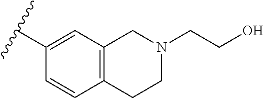 [SMILES: *c1ccc2c(c1)CN(CCO)CC2] | 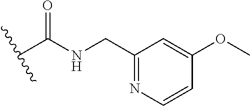 [SMILES: *C(=O)NCc1cc(OC)ccn1] |
| 1231 | 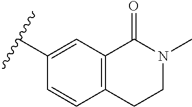 [SMILES: *c1ccc2c(c1)C(=O)N(C)CC2] | 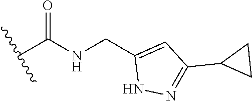 [SMILES: *C(=O)NCc1cc(C2CC2)n[nH]1] |
| 1232 | 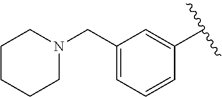 [SMILES: *c1cccc(CN2CCCCC2)c1] | 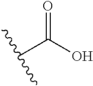 [SMILES: *C(=O)O] |
| 1233 | 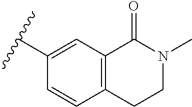 [SMILES: *c1ccc2c(c1)C(=O)N(C)CC2] | 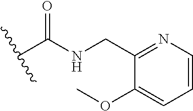 [SMILES: *C(=O)NCc1ncccc1OC] |
| 1234 | 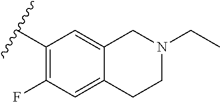 [SMILES: *c1cc2c(cc1F)CCN(CC)C2] | 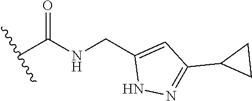 [SMILES: *C(=O)NCc1cc(C2CC2)n[nH]1] |
| 1235 | 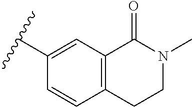 [SMILES: *c1ccc2c(c1)C(=O)N(C)CC2] | 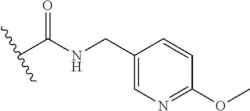 [SMILES: *C(=O)NCc1ccc(OC)nc1] |
| 1236 | 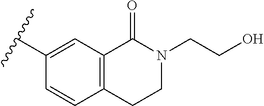 [SMILES: *c1ccc2c(c1)C(=O)N(CCO)CC2] | 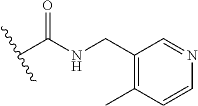 [SMILES: *C(=O)NCc1cnccc1C] |
| 1237 | 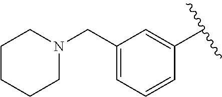 [SMILES: *c1cccc(CN2CCCCC2)c1] | 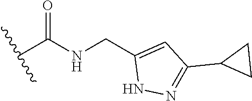 [SMILES: *C(=O)NCc1cc(C2CC2)n[nH]1] |
| 1238 | 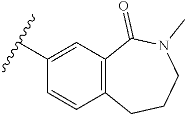 [SMILES: *c1ccc2c(c1)C(=O)N(C)CCC2] | 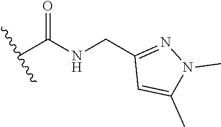 [SMILES: *C(=O)NCc1cc(C)n(C)n1] |
| 1239 | 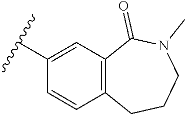 [SMILES: *c1ccc2c(c1)C(=O)N(C)CCC2] | 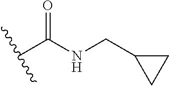 [SMILES: *C(=O)NCC1CC1] |
| 1240 | 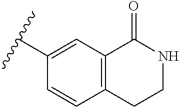 [SMILES: *c1ccc2c(c1)C(=O)NCC2] | 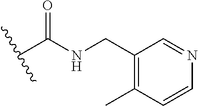 [SMILES: *C(=O)NCc1cnccc1C] |
| 1241 | 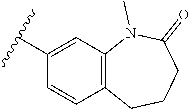 [SMILES: *c1ccc2c(c1)N(C)C(=O)CCC2] | 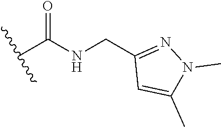 [SMILES: *C(=O)NCc1cc(C)n(C)n1] |
| 1242 | 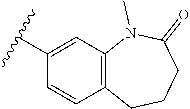 [SMILES: *c1ccc2c(c1)N(C)C(=O)CCC2] | 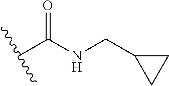 [SMILES: *C(=O)NCC1CC1] |
| 1243 | 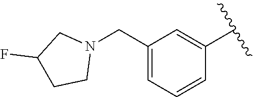 [SMILES: *c1cccc(CN2CCC(F)C2)c1] | 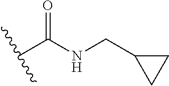 [SMILES: *C(=O)NCC1CC1] |
| 1244 | 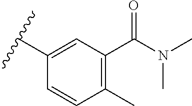 [SMILES: *c1ccc(C)c(C(=O)N(C)C)c1] | 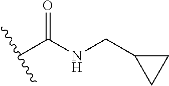 [SMILES: *C(=O)NCC1CC1] |
| 1245 | 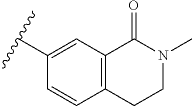 [SMILES: *c1ccc2c(c1)C(=O)N(C)CC2] | 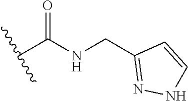 [SMILES: *C(=O)NCc1cc[nH]n1] |
| 1246 | 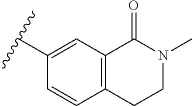 [SMILES: *c1ccc2c(c1)C(=O)N(C)CC2] | 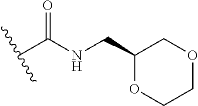 [SMILES: *C(=O)NC[C@H]1COCCO1] |
| 1247 | 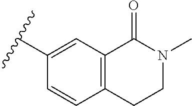 [SMILES: *c1ccc2c(c1)C(=O)N(C)CC2] | 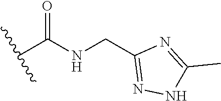 [SMILES: *C(=O)NCc1n[nH]c(C)n1] |
| 1248 | 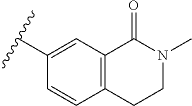 [SMILES: *c1ccc2c(c1)C(=O)N(C)CC2] | 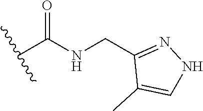 [SMILES: *C(=O)NCc1n[nH]cc1C] |
| 1249 | 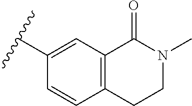 [SMILES: *c1ccc2c(c1)C(=O)N(C)CC2] | 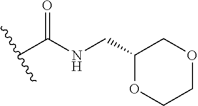 [SMILES: *C(=O)NC[C@@H]1COCCO1] |
| 1250 | 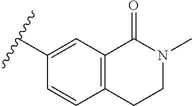 [SMILES: *c1ccc2c(c1)C(=O)N(C)CC2] | 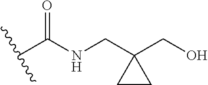 [SMILES: *C(=O)NCC1(CO)CC1] |
| 1251 | 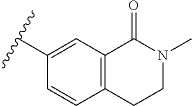 [SMILES: *c1ccc2c(c1)C(=O)N(C)CC2] | 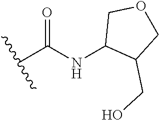 [SMILES: *C(=O)NC1COCC1CO] |
| 1252 | 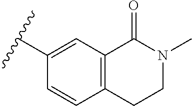 [SMILES: *c1ccc2c(c1)C(=O)N(C)CC2] | 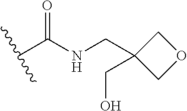 [SMILES: *C(=O)NCC1(CO)COC1] |
| 1253 | 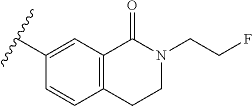 [SMILES: *c1ccc2c(c1)C(=O)N(CCF)CC2] | 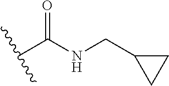 [SMILES: *C(=O)NCC1CC1] |
| 1254 | 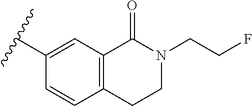 [SMILES: *c1ccc2c(c1)C(=O)N(CCF)CC2] | 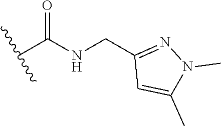 [SMILES: *C(=O)NCc1cc(C)n(C)n1] |
| 1255 | 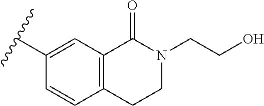 [SMILES: *c1ccc2c(c1)C(=O)N(CCO)CC2] | 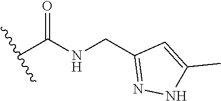 [SMILES: *C(=O)NCc1cc(C)[nH]n1] |
| 1256 | 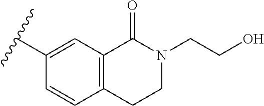 [SMILES: *c1ccc2c(c1)C(=O)N(CCO)CC2] | 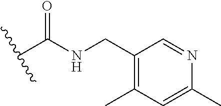 [SMILES: *C(=O)NCc1cnc(C)cc1C] |
| 1257 | 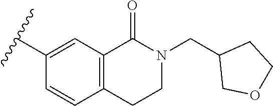 [SMILES: *c1ccc2c(c1)C(=O)N(CC1CCOC1)CC2] | 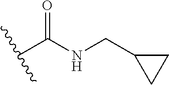 [SMILES: *C(=O)NCC1CC1] |
| 1258 | 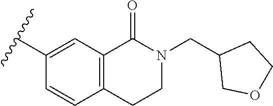 [SMILES: *c1ccc2c(c1)C(=O)N(CC1CCOC1)CC2] | 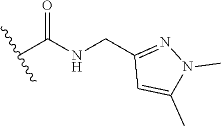 [SMILES: *C(=O)NCc1cc(C)n(C)n1] |
| 1259 | 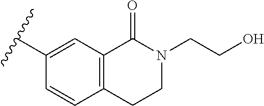 [SMILES: *c1ccc2c(c1)C(=O)N(CCO)CC2] | 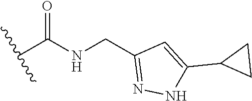 [SMILES: *C(=O)NCc1cc(C2CC2)[nH]n1] |
| 1260 | 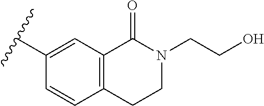 [SMILES: *c1ccc2c(c1)C(=O)N(CCO)CC2] | 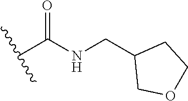 [SMILES: *C(=O)NCC1CCOC1] |
| 1261 | 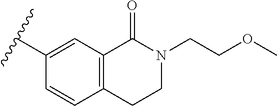 [SMILES: *c1ccc2c(c1)C(=O)N(CCOC)CC2] | 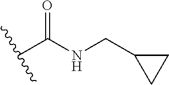 [SMILES: *C(=O)NCC1CC1] |
| 1262 | 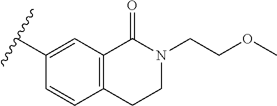 [SMILES: *c1ccc2c(c1)C(=O)N(CCOC)CC2] | 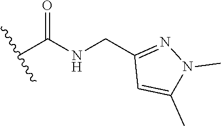 [SMILES: *C(=O)NCc1cc(C)n(C)n1] |
| 1263 | 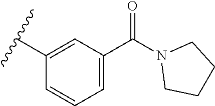 [SMILES: *c1cccc(C(=O)N2CCCC2)c1] | 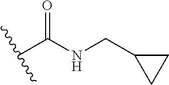 [SMILES: *C(=O)NCC1CC1] |
| 1264 | 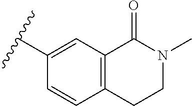 [SMILES: *c1ccc2c(c1)C(=O)N(C)CC2] | 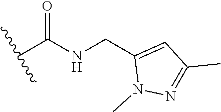 [SMILES: *C(=O)NCc1cc(C)nn1C] |
| 1265 | 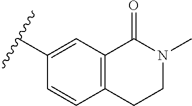 [SMILES: *c1ccc2c(c1)C(=O)N(C)CC2] | 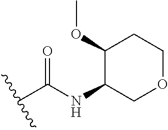 [SMILES: *C(=O)N[C@@H]1COCC[C@@H]1OC] |
| 1266 | 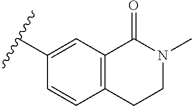 [SMILES: *c1ccc2c(c1)C(=O)N(C)CC2] | 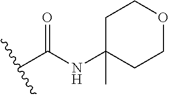 [SMILES: *C(=O)NC1(C)CCOCC1] |
| 1267 | 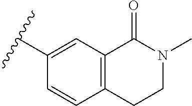 [SMILES: *c1ccc2c(c1)C(=O)N(C)CC2] | 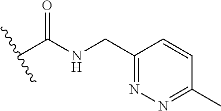 [SMILES: *C(=O)NCc1ccc(C)nn1] |
| 1268 | 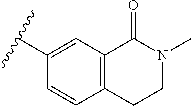 [SMILES: *c1ccc2c(c1)C(=O)N(C)CC2] | 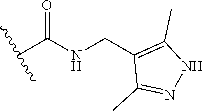 [SMILES: *C(=O)NCc1c(C)n[nH]c1C] |
| 1269 | 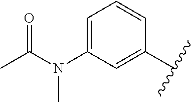 [SMILES: *c1cccc(N(C)C(C)=O)c1] | 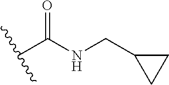 [SMILES: *C(=O)NCC1CC1] |
| 1270 | 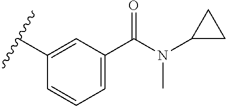 [SMILES: *c1cccc(C(=O)N(C)C2CC2)c1] | 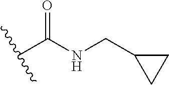 [SMILES: *C(=O)NCC1CC1] |
| 1271 | 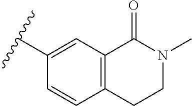 [SMILES: *c1ccc2c(c1)C(=O)N(C)CC2] | 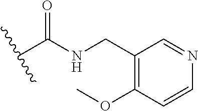 [SMILES: *C(=O)NCc1cnccc1OC] |
| 1272 | 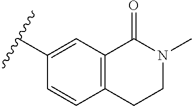 [SMILES: *c1ccc2c(c1)C(=O)N(C)CC2] | 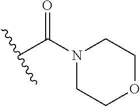 [SMILES: *C(=O)N1CCOCC1] |
| 1273 | 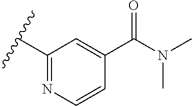 [SMILES: *c1cc(C(=O)N(C)C)ccn1] | 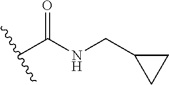 [SMILES: *C(=O)NCC1CC1] |
| 1274 | 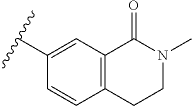 [SMILES: *c1ccc2c(c1)C(=O)N(C)CC2] | 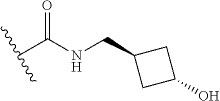 [SMILES: *C(=O)NC[C@H]1C[C@H](O)C1] |
| 1275 | 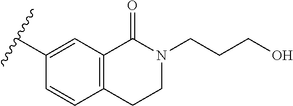 [SMILES: *c1ccc2c(c1)C(=O)N(CCCO)CC2] | 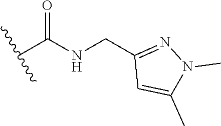 [SMILES: *C(=O)NCc1cc(C)n(C)n1] |
| 1276 | 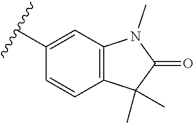 [SMILES: *c1ccc2c(c1)N(C)C(=O)C2(C)C] | 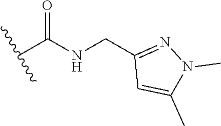 [SMILES: *C(=O)NCc1cc(C)n(C)n1] |
| 1277 | 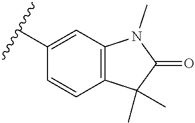 [SMILES: *c1ccc2c(c1)N(C)C(=O)C2(C)C] | 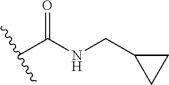 [SMILES: *C(=O)NCC1CC1] |
| 1278 | 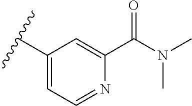 [SMILES: *c1ccnc(C(=O)N(C)C)c1] | 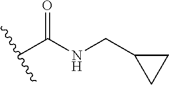 [SMILES: *C(=O)NCC1CC1] |
| 1279 | 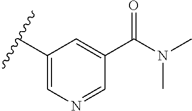 [SMILES: *c1cncc(C(=O)N(C)C)c1] | 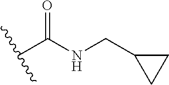 [SMILES: *C(=O)NCC1CC1] |
| 1280 | 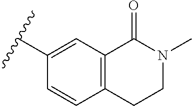 [SMILES: *c1ccc2c(c1)C(=O)N(C)CC2] | 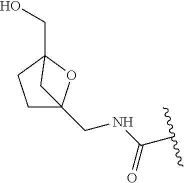 [SMILES: *C(=O)NCC12CCC(CO)(C1)O2] |
| 1281 | 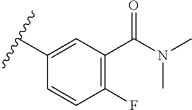 [SMILES: *c1ccc(F)c(C(=O)N(C)C)c1] | 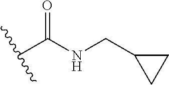 [SMILES: *C(=O)NCC1CC1] |
| 1282 | 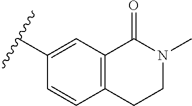 [SMILES: *c1ccc2c(c1)C(=O)N(C)CC2] | 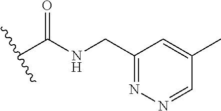 [SMILES: *C(=O)NCc1cc(C)cnn1] |
| 1283 | 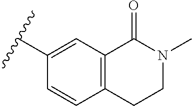 [SMILES: *c1ccc2c(c1)C(=O)N(C)CC2] | 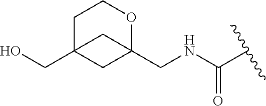 [SMILES: *C(=O)NCC12CC(CO)(CCO1)C2] |
| 1284 | 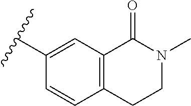 [SMILES: *c1ccc2c(c1)C(=O)N(C)CC2] | 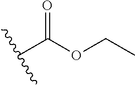 [SMILES: *C(=O)OCC] |
| 1285 | 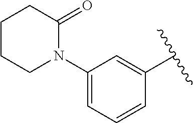 [SMILES: *c1cccc(N2CCCCC2=O)c1] | 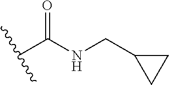 [SMILES: *C(=O)NCC1CC1] |
| 1286 | 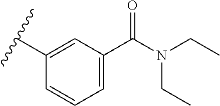 [SMILES: *c1cccc(C(=O)N(CC)CC)c1] | 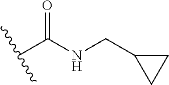 [SMILES: *C(=O)NCC1CC1] |
| 1287 | 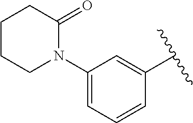 [SMILES: *c1cccc(N2CCCCC2=O)c1] | 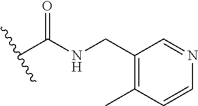 [SMILES: *C(=O)NCc1cnccc1C] |
| 1288 | 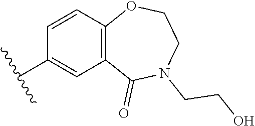 [SMILES: *c1ccc2c(c1)C(=O)N(CCO)CCO2] | 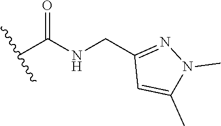 [SMILES: *C(=O)NCc1cc(C)n(C)n1] |
| 1289 | 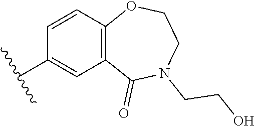 [SMILES: *c1ccc2c(c1)C(=O)N(CCO)CCO2] | 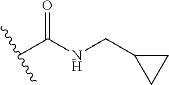 [SMILES: *C(=O)NCC1CC1] |
| 1290 | 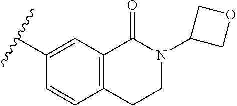 [SMILES: *c1ccc2c(c1)C(=O)N(C1COC1)CC2] | 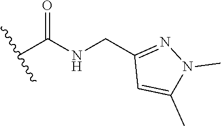 [SMILES: *C(=O)NCc1cc(C)n(C)n1] |
| 1292 | 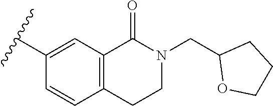 [SMILES: *c1ccc2c(c1)C(=O)N(CC1CCCO1)CC2] | 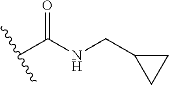 [SMILES: *C(=O)NCC1CC1] |
| 1293 | 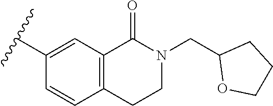 [SMILES: *c1ccc2c(c1)C(=O)N(CC1CCCO1)CC2] | 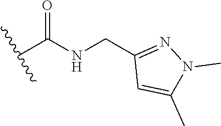 [SMILES: *C(=O)NCc1cc(C)n(C)n1] |
| 1294 | 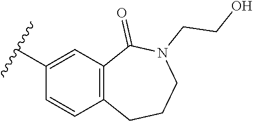 [SMILES: *c1ccc2c(c1)C(=O)N(CCO)CCC2] | 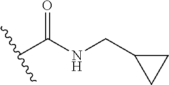 [SMILES: *C(=O)NCC1CC1] |
| 1295 | 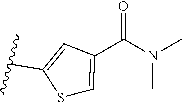 [SMILES: *c1cc(C(=O)N(C)C)cs1] | 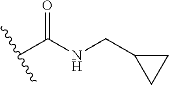 [SMILES: *C(=O)NCC1CC1] |
| 1296 | 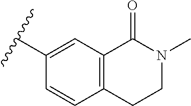 [SMILES: *c1ccc2c(c1)C(=O)N(C)CC2] | 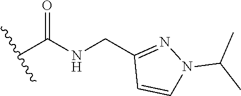 [SMILES: *C(=O)NCc1ccn(C(C)C)n1] |
| 1297 | 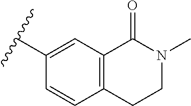 [SMILES: *c1ccc2c(c1)C(=O)N(C)CC2] | 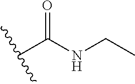 [SMILES: *C(=O)NCC] |

TABLE 1c

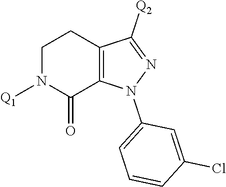

| Example | | |
|---|---|---|
| Number | Q1 | Q2 |
| 1298 | 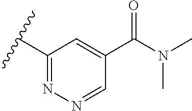 [SMILES: *c1cc(C(=O)N(C)C)cnn1] | 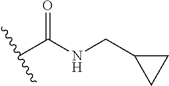 [SMILES: *C(=O)NCC1CC1] |
| 1299 | 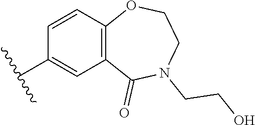 [SMILES: *c1ccc2c(c1)C(=O)N(CCO)CCO2] | 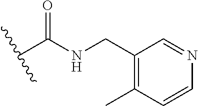 [SMILES: *C(=O)NCc1cnccc1C] |
| 1300 | 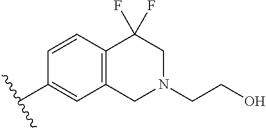 [SMILES: *c1ccc2c(c1)CN(CCO)CC2(F)F] | 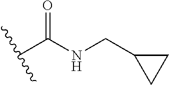 [SMILES: *C(=O)NCC1CC1] |
| 1301 | 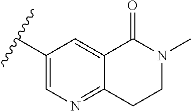 [SMILES: *c1cnc2c(c1)C(=O)N(C)CC2] | 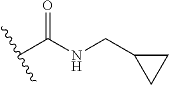 [SMILES: *C(=O)NCC1CC1] |
| 1302 | 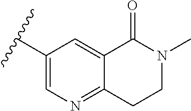 [SMILES: *c1cnc2c(c1)C(=O)N(C)CC2] | 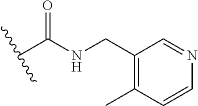 [SMILES: *C(=O)NCc1cnccc1C] |
| 1303 | 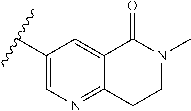 [SMILES: *c1cnc2c(c1)C(=O)N(C)CC2] | 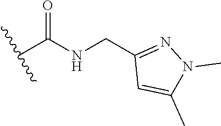 [SMILES: *C(=O)NCc1cc(C)n(C)n1] |
| 1304 | 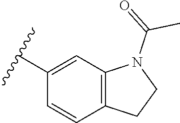 [SMILES: *c1ccc2c(c1)N(C(C)=O)CC2] | 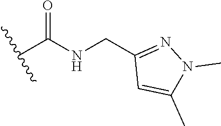 [SMILES: *C(=O)NCc1cc(C)n(C)n1] |
| 1305 | 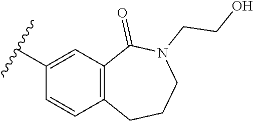 [SMILES: *c1ccc2c(c1)C(=O)N(CCO)CCC2] | 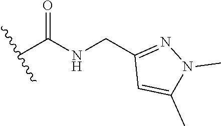 [SMILES: *C(=O)NCc1cc(C)n(C)n1] |
| 1306 | 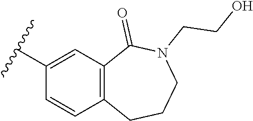 [SMILES: *c1ccc2c(c1)C(=O)N(CCO)CCC2] | 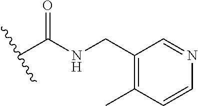 [SMILES: *C(=O)NCc1cnccc1C] |
| 1307 | 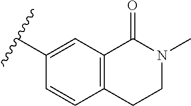 [SMILES: *c1ccc2c(c1)C(=O)N(C)CC2] | 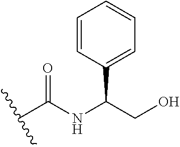 [SMILES: *C(=O)N[C@H](CO)c1ccccc1] |
| 1308 | 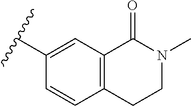 [SMILES: *c1ccc2c(c1)C(=O)N(C)CC2] | 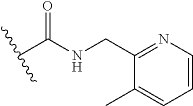 [SMILES: *C(=O)NCc1ncccc1C] |
| 1309 | 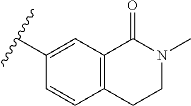 [SMILES: *c1ccc2c(c1)C(=O)N(C)CC2] | 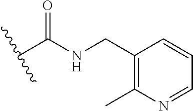 [SMILES: *C(=O)NCc1cccnc1C] |
| 1310 | 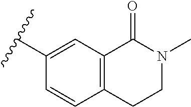 [SMILES: *c1ccc2c(c1)C(=O)N(C)CC2] | 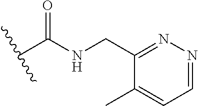 [SMILES: *C(=O)NCc1nnccc1C] |
| 1311 | 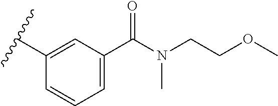 [SMILES: *c1cccc(C(=O)N(C)CCOC)c1] | 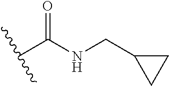 [SMILES: *C(=O)NCC1CC1] |
| 1312 | 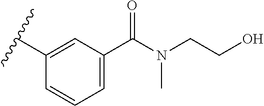 [SMILES: *c1cccc(C(=O)N(C)CCO)c1] | 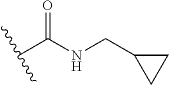 [SMILES: *C(=O)NCC1CC1] |
| 1313 | 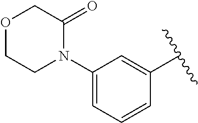 [SMILES: *c1cccc(N2CCOCC2=O)c1] | 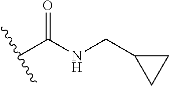 [SMILES: *C(=O)NCC1CC1] |
| 1314 | 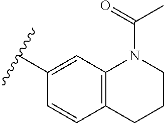 [SMILES: *c1ccc2c(c1)N(C(C)=O)CCC2] | 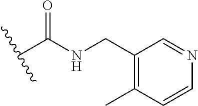 [SMILES: *C(=O)NCc1cnccc1C] |
| 1316 | 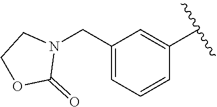 [SMILES: *c1cccc(CN2CCOC2=O)c1] | 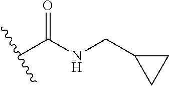 [SMILES: *C(=O)NCC1CC1] |
| 1317 | 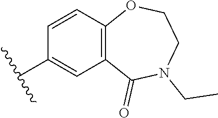 [SMILES: *c1ccc2c(c1)C(=O)N(CC)CCO2] | 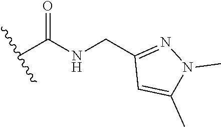 [SMILES: *C(=O)NCc1cc(C)n(C)n1] |
| 1318 | 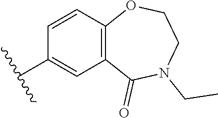 [SMILES: *c1ccc2c(c1)C(=O)N(CC)CCO2] | 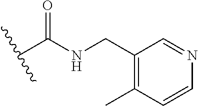 [SMILES: *C(=O)NCc1cnccc1C] |
| 1319 | 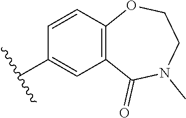 [SMILES: *c1ccc2c(c1)C(=O)N(C)CCO2] | 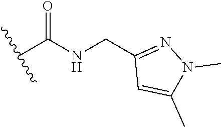 [SMILES: *C(=O)NCc1cc(C)n(C)n1] |
| 1320 | 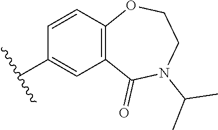 [SMILES: *c1ccc2c(c1)C(=O)N(C(C)C)CCO2] | 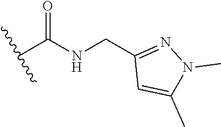 [SMILES: *C(=O)NCc1cc(C)n(C)n1] |
| 1321 | 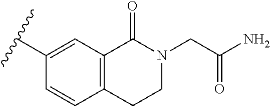 [SMILES: *c1ccc2c(c1)C(=O)N(CC(N)=O)CC2] | 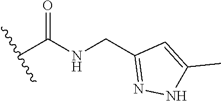 [SMILES: *C(=O)NCc1cc(C)[nH]n1] |
| 1322 | 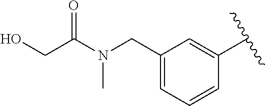 [SMILES: *c1cccc(CN(C)C(=O)CO)c1] | 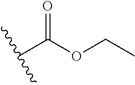 [SMILES: *C(=O)OCC] |
| 1323 | 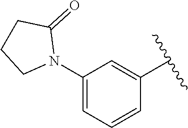 [SMILES: *c1cccc(N2CCCC2=O)c1] | 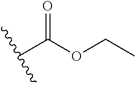 [SMILES: *C(=O)OCC] |
| 1324 | 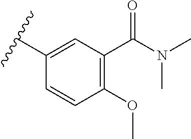 [SMILES: *c1ccc(OC)c(C(=O)N(C)C)c1] | 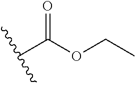 [SMILES: *C(=O)OCC] |
| 1325 | 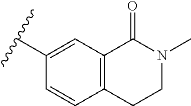 [SMILES: *c1ccc2c(c1)C(=O)N(C)CC2] | 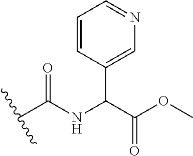 [SMILES: *C(=O)NC(C(=O)OC)c1cccnc1] |
| 1326 | 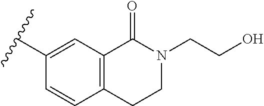 [SMILES: *c1ccc2c(c1)C(=O)N(CCO)CC2] | 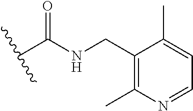 [SMILES: *C(=O)NCc1c(C)ccnc1C] |
| 1327 | 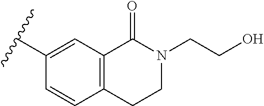 [SMILES: *c1ccc2c(c1)C(=O)N(CCO)CC2] | 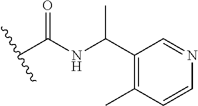 [SMILES: *C(=O)NC(C)c1cnccc1C] |
| 1328 | 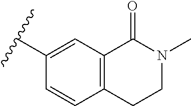 [SMILES: *c1ccc2c(c1)C(=O)N(C)CC2] | 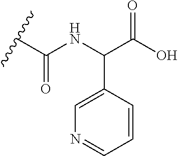 [SMILES: *C(=O)NC(C(=O)O)c1cccnc1] |
| 1329 | 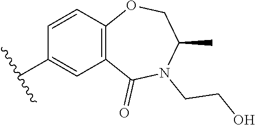 [SMILES: *c1ccc2c(c1)C(=O)N(CCO)[C@H](C)CO2] | 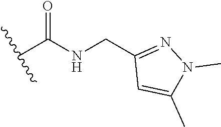 [SMILES: *C(=O)NCc1cc(C)n(C)n1] |
| 1330 | 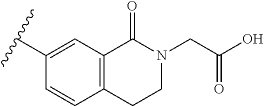 [SMILES: *c1ccc2c(c1)C(=O)N(CC(=O)O)CC2] | 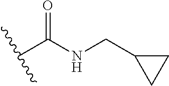 [SMILES: *C(=O)NCC1CC1] |
| 1331 | 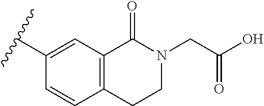 [SMILES: *c1ccc2c(c1)C(=O)N(CC(=O)O)CC2] | 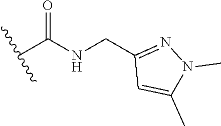 [SMILES: *C(=O)NCc1cc(C)n(C)n1] |
| 1332 | 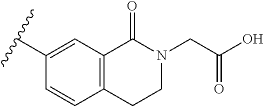 [SMILES: *c1ccc2c(c1)C(=O)N(CC(=O)O)CC2] | 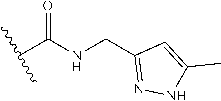 [SMILES: *C(=O)NCc1cc(C)[nH]n1] |
| 1333 | 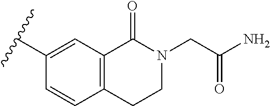 [SMILES: *c1ccc2c(c1)C(=O)N(CC(N)=O)CC2] | 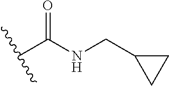 [SMILES: *C(=O)NCC1CC1] |
| 1334 | 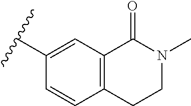 [SMILES: *c1ccc2c(c1)C(=O)N(C)CC2] | 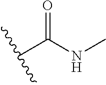 [SMILES: *C(=O)NC] |
| 1335 | 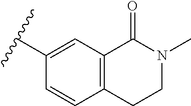 [SMILES: *c1ccc2c(c1)C(=O)N(C)CC2] | 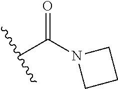 [SMILES: *C(=O)N1CCC1] |
| 1336 | 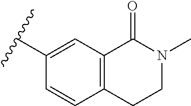 [SMILES: *c1ccc2c(c1)C(=O)N(C)CC2] | 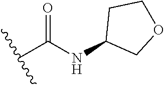 [SMILES: *C(=O)N[C@H]1CCOC1] |
| 1337 | 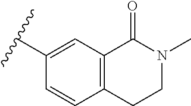 [SMILES: *c1ccc2c(c1)C(=O)N(C)CC2] | 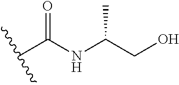 [SMILES: *C(=O)N[C@H](C)CO] |
| 1338 | 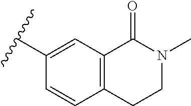 [SMILES: *c1ccc2c(c1)C(=O)N(C)CC2] | 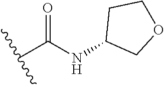 [SMILES: *C(=O)N[C@@H]1CCOC1] |
| 1339 | 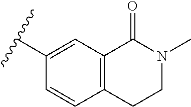 [SMILES: *c1ccc2c(c1)C(=O)N(C)CC2] | 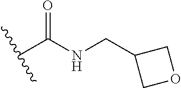 [SMILES: *C(=O)NCC1COC1] |
| 1340 | 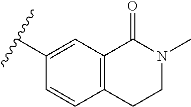 [SMILES: *c1ccc2c(c1)C(=O)N(C)CC2] | 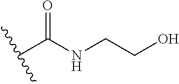 [SMILES: *C(=O)NCCO] |
| 1341 | 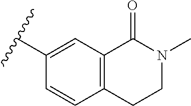 [SMILES: *c1ccc2c(c1)C(=O)N(C)CC2] | 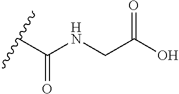 [SMILES: *C(=O)NCC(=O)O] |
| 1342 | 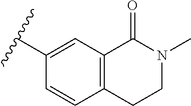 [SMILES: *c1ccc2c(c1)C(=O)N(C)CC2] | 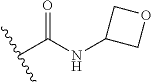 [SMILES: *C(=O)NC1COC1] |
| 1343 | 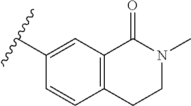 [SMILES: *c1ccc2c(c1)C(=O)N(C)CC2] | 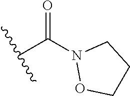 [SMILES: *C(=O)N1CCCO1] |
| 1344 | 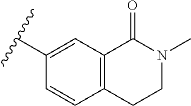 [SMILES: *c1ccc2c(c1)C(=O)N(C)CC2] | 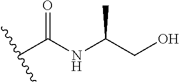 [SMILES: *C(=O)N[C@@H](C)CO] |
| 1345 | 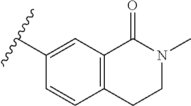 [SMILES: *c1ccc2c(c1)C(=O)N(C)CC2] | 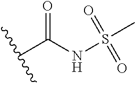 [SMILES: *C(=O)NS(C)(=O)=O] |
| 1346 | 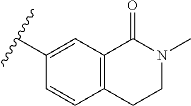 [SMILES: *c1ccc2c(c1)C(=O)N(C)CC2] | 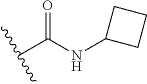 [SMILES: *C(=O)NC1CCC1] |
| 1347 | 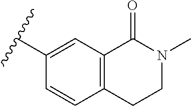 [SMILES: *c1ccc2c(c1)C(=O)N(C)CC2] | 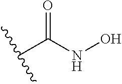 [SMILES: *C(=O)NO] |
| 1350 | 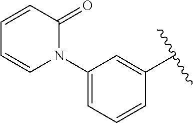 [SMILES: *c1cccc(-n2ccccc2=O)c1] | 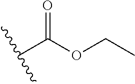 [SMILES: *C(=O)OCC] |
| 1351 | 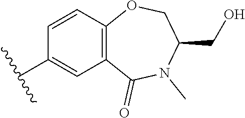 [SMILES: *c1ccc2c(c1)C(=O)N(C)[C@H](CO)CO2] | 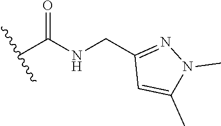 [SMILES: *C(=O)NCc1cc(C)n(C)n1] |
| 1352 | 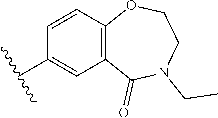 [SMILES: *c1ccc2c(c1)C(=O)N(CC)CCO2] | 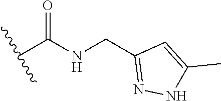 [SMILES: *C(=O)NCc1cc(C)[nH]n1] |
| 1353 | 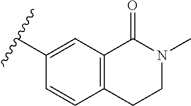 [SMILES: *c1ccc2c(c1)C(=O)N(C)CC2] | 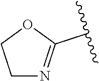 [SMILES: *C1=NCCO1] |
| 1354 | 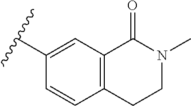 [SMILES: *c1ccc2c(c1)C(=O)N(C)CC2] | 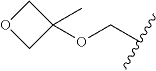 [SMILES: *COC1(C)COC1] |
| 1355 | 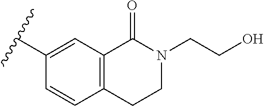 [SMILES: *c1ccc2c(c1)C(=O)N(CCO)CC2] | 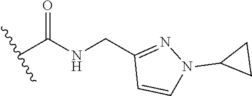 [SMILES: *C(=O)NCc1ccn(C2CC2)n1] |
| 1356 | 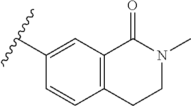 [SMILES: *c1ccc2c(c1)C(=O)N(C)CC2] | 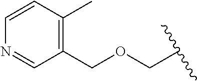 [SMILES: *COCc1cnccc1C] |
| 1357 | 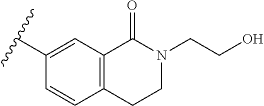 [SMILES: *c1ccc2c(c1)C(=O)N(CCO)CC2] | 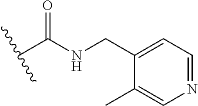 [SMILES: *C(=O)NCc1ccncc1C] |
| 1358 | 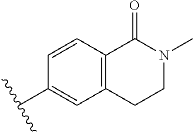 [SMILES: *c1ccc2c(c1)CCN(C)C2=O] | 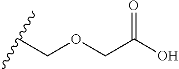 [SMILES: *COCC(=O)O] |
| 1359 | 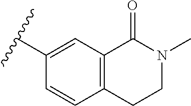 [SMILES: *c1ccc2c(c1)C(=O)N(C)CC2] | 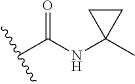 [SMILES: *C(=O)NC1(C)CC1] |
| 1360 | 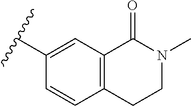 [SMILES: *c1ccc2c(c1)C(=O)N(C)CC2] | 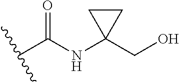 [SMILES: *C(=O)NC1(CO)CC1] |
| 1361 | 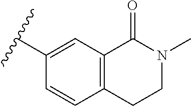 [SMILES: *c1ccc2c(c1)C(=O)N(C)CC2] | 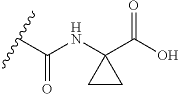 [SMILES: *C(=O)NC1(C(=O)O)CC1] |
| 1362 | 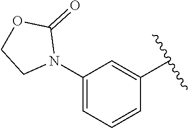 [SMILES: *c1cccc(N2CCOC2=O)c1] | 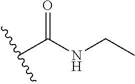 [SMILES: *C(=O)NCC] |
| 1363 | 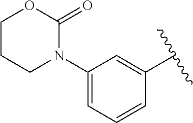 [SMILES: *c1cccc(N2CCCOC2=O)c1] | 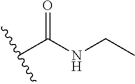 [SMILES: *C(=O)NCC] |
| 1364 | 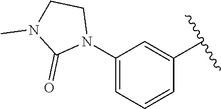 [SMILES: *c1cccc(N2CCN(C)C2=O)c1] | 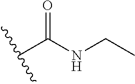 [SMILES: *C(=O)NCC] |
| 1365 | 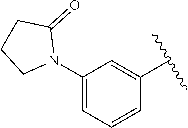 [SMILES: *c1cccc(N2CCCC2=O)c1] | 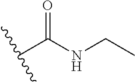 [SMILES: *C(=O)NCC] |
| 1366 | 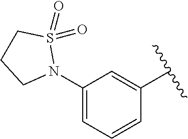 [SMILES: *c1cccc(N2CCCS2(=O)=O)c1] | 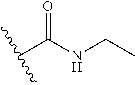 [SMILES: *C(=O)NCC] |
| 1367 | 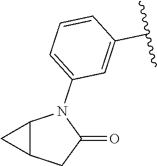 [SMILES: *c1cccc(N2C(=O)CC3CC32)c1] | 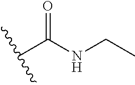 [SMILES: *C(=O)NCC] |
| 1368 | 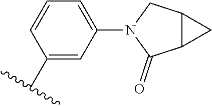 [SMILES: *c1cccc(N2CC3CC3C2=O)c1] | 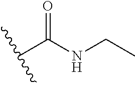 [SMILES: *C(=O)NCC] |
| 1369 | 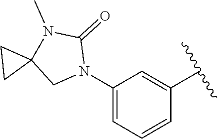 [SMILES: *c1cccc(N2CC3(CC3)N(C)C2=O)c1] | 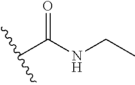 [SMILES: *C(=O)NCC] |
| 1370 | 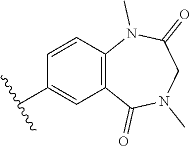 [SMILES: *c1ccc2c(c1)C(=O)N(C)CC(=O)N2C] | 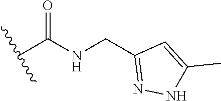 [SMILES: *C(=O)NCc1cc(C)[nH]n1] |
| 1371 | 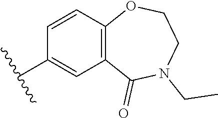 [SMILES: *c1ccc2c(c1)C(=O)N(CC)CCO2] | 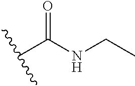 [SMILES: *C(=O)NCC] |
| 1372 | 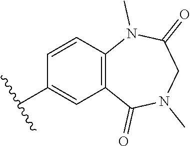 [SMILES: *c1ccc2c(c1)C(=O)N(C)CC(=O)N2C] | 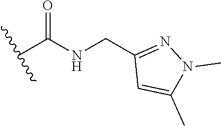 [SMILES: *C(=O)NCc1cc(C)n(C)n1] |
| 1373 | 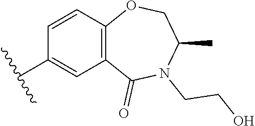 [SMILES: *c1ccc2c(c1)C(=O)N(CCO)[C@H](C)CO2] | 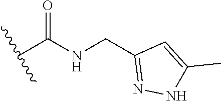 [SMILES: *C(=O)NCc1cc(C)[nH]n1] |
| 1374 | 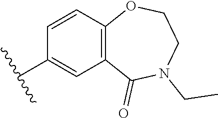 [SMILES: *c1ccc2c(c1)C(=O)N(CC)CCO2] | 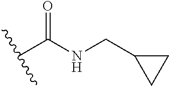 [SMILES: *C(=O)NCC1CC1] |
| 1375 | 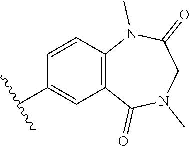 [SMILES: *c1ccc2c(c1)C(=O)N(C)CC(=O)N2C] | 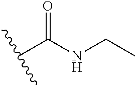 [SMILES: *C(=O)NCC] |
| 1376 | 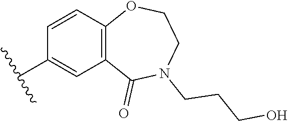 [SMILES: *c1ccc2c(c1)C(=O)N(CCCO)CCO2] | 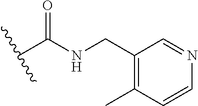 [SMILES: *C(=O)NCc1cnccc1C] |
| 1377 | 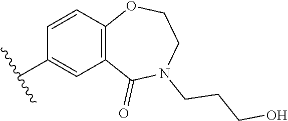 [SMILES: *c1ccc2c(c1)C(=O)N(CCCO)CCO2] | 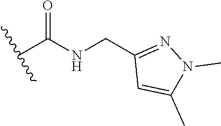 [SMILES: *C(=O)NCc1cc(C)n(C)n1] |
| 1378 | 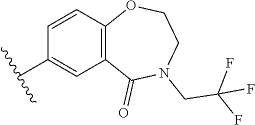 [SMILES: *c1ccc2c(c1)C(=O)N(CC(F)(F)F)CCO2] | 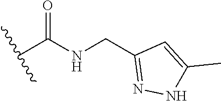 [SMILES: *C(=O)NCc1cc(C)[nH]n1] |
| 1379 | 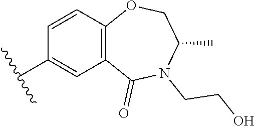 [SMILES: *c1ccc2c(c1)C(=O)N(CCO)[C@@H](C)CO2] | 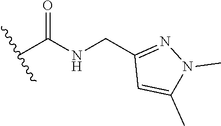 [SMILES: *C(=O)NCc1cc(C)n(C)n1] |
| 1380 | 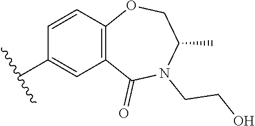 [SMILES: *c1ccc2c(c1)C(=O)N(CCO)[C@@H](C)CO2] | 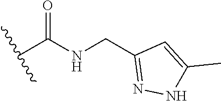 [SMILES: *C(=O)NCc1cc(C)[nH]n1] |
| 1381 | 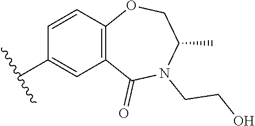 [SMILES: *c1ccc2c(c1)C(=O)N(CCO)[C@@H](C)CO2] | 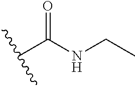 [SMILES: *C(=O)NCC] |
| 1382 | 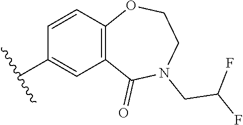 [SMILES: *c1ccc2c(c1)C(=O)N(CC(F)F)CCO2] | 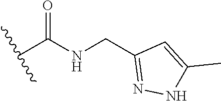 [SMILES: *C(=O)NCc1cc(C)[nH]n1] |
| 1383 | 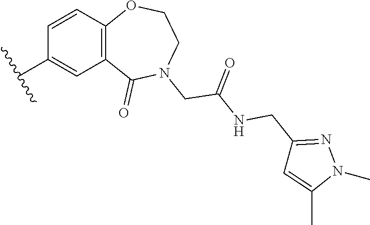 [SMILES: *c1ccc2c(c1)C(=O)N(CC(=O)NCc1cc(C)n(C)n1)CCO2] | 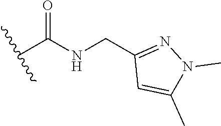 [SMILES: *C(=O)NCc1cc(C)n(C)n1] |
| 1384 | 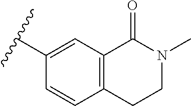 [SMILES: *c1ccc2c(c1)C(=O)N(C)CC2] | 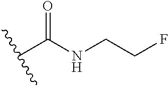 [SMILES: *C(=O)NCCF] |
| 1385 | 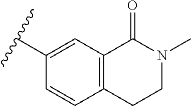 [SMILES: *c1ccc2c(c1)C(=O)N(C)CC2] | 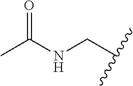 [SMILES: *CNC(C)=O] |
| 1386 | 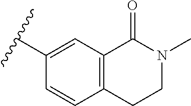 [SMILES: *c1ccc2c(c1)C(=O)N(C)CC2] | 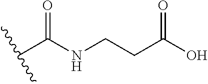 [SMILES: *C(=O)NCCC(=O)O] |
| 1387 | 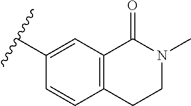 [SMILES: *c1ccc2c(c1)C(=O)N(C)CC2] | 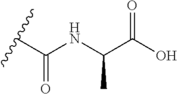 [SMILES: *C(=O)N[C@H](C)C(=O)O] |
| 1388 | 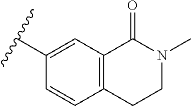 [SMILES: *c1ccc2c(c1)C(=O)N(C)CC2] | 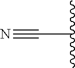 [SMILES: *C#N] |
| 1389 | 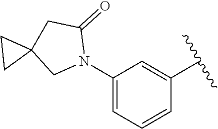 [SMILES: *c1cccc(N2CC3(CC3)CC2=O)c1] | 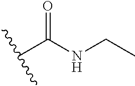 [SMILES: *C(=O)NCC] |
| 1390 | 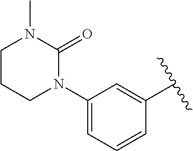 [SMILES: *c1cccc(N2CCCN(C)C2=O)c1] | 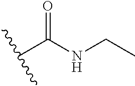 [SMILES: *C(=O)NCC] |

TABLE 1d

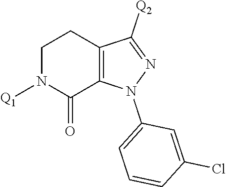

| Example | | |
|---|---|---|
| Number | Q1 | Q2 |
| 1391 | 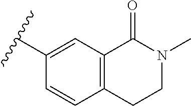 [SMILES: *c1ccc2c(c1)C(=O)N(C)CC2] | 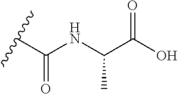 [SMILES: *C(=O)N[C@@H](C)C(=O)O] |
| 1392 | 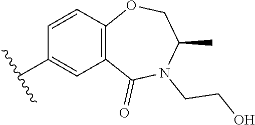 [SMILES: *c1ccc2c(c1)C(=O)N(CCO)[C@H](C)CO2] | 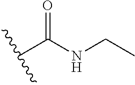 [SMILES: *C(=O)NCC] |
| 1393 | 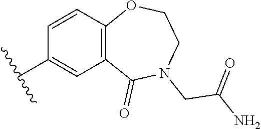 [SMILES: *c1ccc2c(c1)C(=O)N(CC(N)=O)CCO2] | 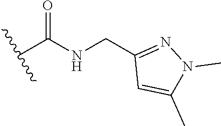 [SMILES: *C(=O)NCc1cc(C)n(C)n1] |
| 1394 | 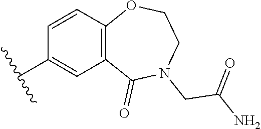 [SMILES: *c1ccc2c(c1)C(=O)N(CC(N)=O)CCO2] | 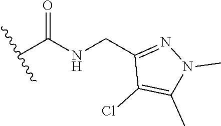 [SMILES: *C(=O)NCc1nn(C)c(C)c1Cl] |
| 1395 | 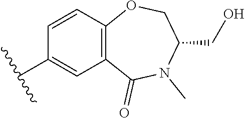 [SMILES: *c1ccc2c(c1)C(=O)N(C)[C@@H](CO)CO2] | 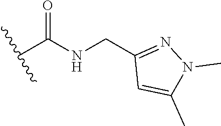 [SMILES: *C(=O)NCc1cc(C)n(C)n1] |
| 1396 | 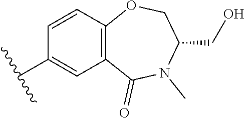 [SMILES: *c1ccc2c(c1)C(=O)N(C)[C@@H](CO)CO2] | 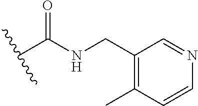 [SMILES: *C(=O)NCc1cnccc1C] |
| 1397 | 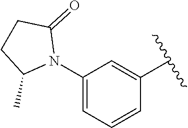 [SMILES: *c1cccc(N2C(=O)CC[C@H]2C)c1] | 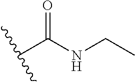 [SMILES: *C(=O)NCC] |
| 1398 | 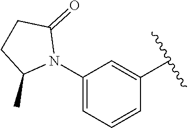 [SMILES: *c1cccc(N2C(=O)CC[C@@H]2C)c1] | 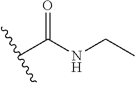 [SMILES: *C(=O)NCC] |
| 1399 | 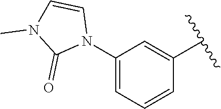 [SMILES: *c1cccc(-n2ccn(C)c2=O)c1] | 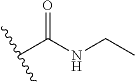 [SMILES: *C(=O)NCC] |
| 1400 | 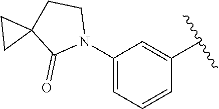 [SMILES: *c1cccc(N2CCC3(CC3)C2=O)c1] | 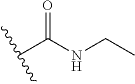 [SMILES: *C(=O)NCC] |
| 1401 | 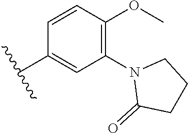 [SMILES: *c1ccc(OC)c(N2CCCC2=O)c1] | 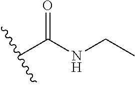 [SMILES: *C(=O)NCC] |
| 1402 | 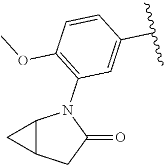 [SMILES: *c1ccc(OC)c(N2C(=O)CC3CC32)c1] | 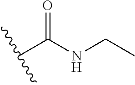 [SMILES: *C(=O)NCC] |
| 1403 | 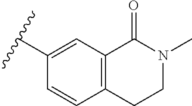 [SMILES: *c1ccc2c(c1)C(=O)N(C)CC2] | 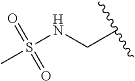 [SMILES: *CNS(C)(=O)=O] |
| 1404 | 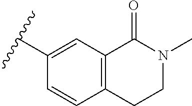 [SMILES: *c1ccc2c(c1)C(=O)N(C)CC2] | 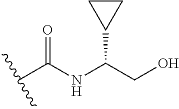 [SMILES: *C(=O)N[C@@H](CO)C1CC1] |
| 1405 | 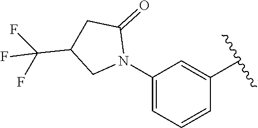 [SMILES: *c1cccc(N2CC(C(F)(F)F)CC2=O)c1] | 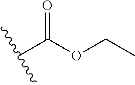 [SMILES: *C(=O)OCC] |
| 1406 | 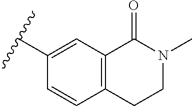 [SMILES: *c1ccc2c(c1)C(=O)N(C)CC2] | 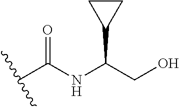 [SMILES: *C(=O)N[C@H](CO)C1CC1] |
| 1407 | 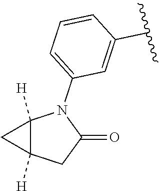 [SMILES: *c1cccc(N2C(=O)C[C@H]3C[C@H]32)c1] | 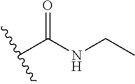 [SMILES: *C(=O)NCC] |
| 1408 | 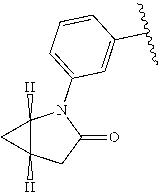 [SMILES: *c1cccc(N2C(=O)C[C@@H]3C[C@@H]32)c1] | 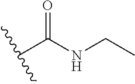 [SMILES: *C(=O)NCC] |
| 1409 | 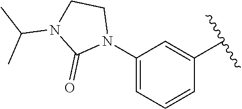 [SMILES: *c1cccc(N2CCN(C(C)C)C2=O)c1] | 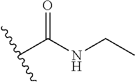 [SMILES: *C(=O)NCC] |
| 1410 | 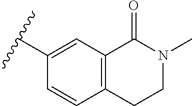 [SMILES: *c1ccc2c(c1)C(=O)N(C)CC2] | 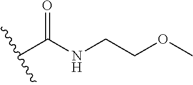 [SMILES: *C(=O)NCCOC] |
| 1411 | 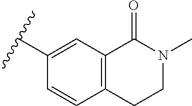 [SMILES: *c1ccc2c(c1)C(=O)N(C)CC2] | 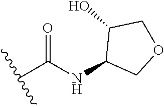 [SMILES: *C(=O)N[C@@H]1COC[C@H]1O] |
| 1412 | 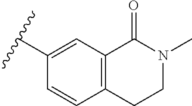 [SMILES: *c1ccc2c(c1)C(=O)N(C)CC2] | 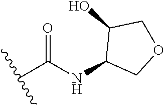 [SMILES: *C(=O)N[C@@H]1COC[C@@H]1O] |
| 1413 | 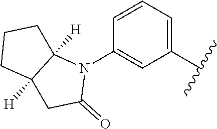 [SMILES: *c1cccc(N2C(=O)C[C@H]3CCC[C@H]32)c1] | 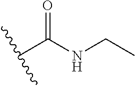 [SMILES: *C(=O)NCC] |
| 1414 | 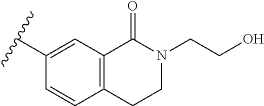 [SMILES: *c1ccc2c(c1)C(=O)N(CCO)CC2] | 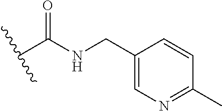 [SMILES: *C(=O)NCc1ccc(C)nc1] |
| 1415 | 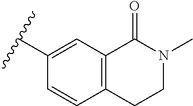 [SMILES: *c1ccc2c(c1)C(=O)N(C)CC2] | 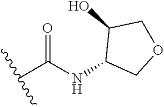 [SMILES: *C(=O)N[C@H]1COC[C@@H]1O] |
| 1416 | 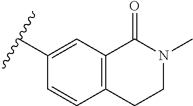 [SMILES: *c1ccc2c(c1)C(=O)N(C)CC2] | 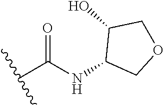 [SMILES: *C(=O)N[C@H]1COC[C@H]1O] |
| 1417 | 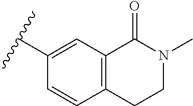 [SMILES: *c1ccc2c(c1)C(=O)N(C)CC2] | 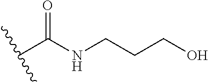 [SMILES: *C(=O)NCCCO] |
| 1418 | 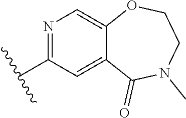 [SMILES: *c1cc2c(cn1)OCCN(C)C2=O] | 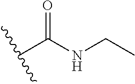 [SMILES: *C(=O)NCC] |
| 1419 | 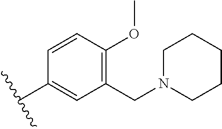 [SMILES: *c1ccc(OC)c(CN2CCCCC2)c1] | 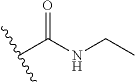 [SMILES: *C(=O)NCC] |
| 1420 | 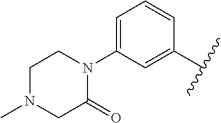 [SMILES: *c1cccc(N2CCN(C)CC2=O)c1] | 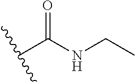 [SMILES: *C(=O)NCC] |
| 1421 | 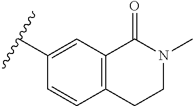 [SMILES: *c1ccc2c(c1)C(=O)N(C)CC2] | 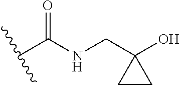 [SMILES: *C(=O)NCC1(O)CC1] |
| 1422 | 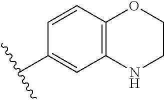 [SMILES: *c1ccc2c(c1)NCCO2] | 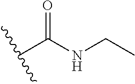 [SMILES: *C(=O)NCC] |
| 1423 | 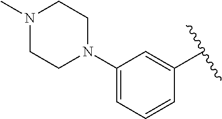 [SMILES: *c1cccc(N2CCN(C)CC2)c1] | 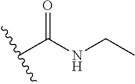 [SMILES: *C(=O)NCC] |
| 1424 | 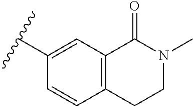 [SMILES: *c1ccc2c(c1)C(=O)N(C)CC2] | 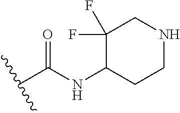 [SMILES: *C(=O)NC1CCNCC1(F)F] |
| 1425 | 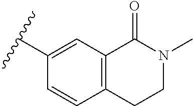 [SMILES: *c1ccc2c(c1)C(=O)N(C)CC2] | 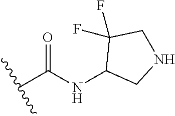 [SMILES: *C(=O)NC1CNCC1(F)F] |
| 1426 | 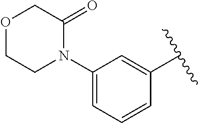 [SMILES: *c1cccc(N2CCOCC2=O)c1] | 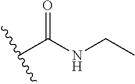 [SMILES: *C(=O)NCC] |
| 1427 | 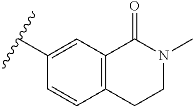 [SMILES: *c1ccc2c(c1)C(=O)N(C)CC2] | 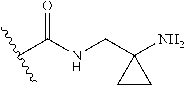 [SMILES: *C(=O)NCC1(N)CC1] |
| 1428 | 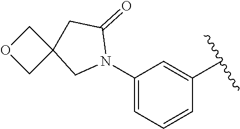 [SMILES: *c1cccc(N2CC3(COC3)CC2=O)c1] | 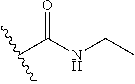 [SMILES: *C(=O)NCC] |
| 1429 | 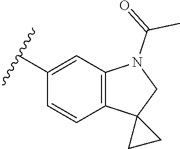 [SMILES: *c1ccc2c(c1)N(C(C)=O)CC21CC1] | 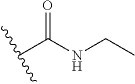 [SMILES: *C(=O)NCC] |
| 1430 | 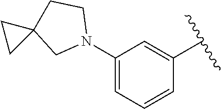 [SMILES: *c1cccc(N2CCC3(CC3)C2)c1] | 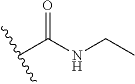 [SMILES: *C(=O)NCC] |
| 1431 | 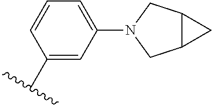 [SMILES: *c1cccc(N2CC3CC3C2)c1] | 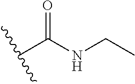 [SMILES: *C(=O)NCC] |
| 1432 | 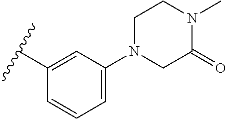 [SMILES: *c1cccc(N2CCN(C)C(=O)C2)c1] | 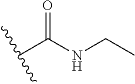 [SMILES: *C(=O)NCC] |
| 1433 | 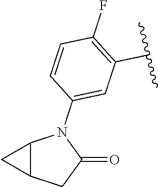 [SMILES: *c1cc(N2C(=O)CC3CC32)ccc1F] | 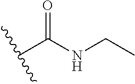 [SMILES: *C(=O)NCC] |
| 1434 | 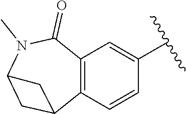 [SMILES: *c1ccc2c(c1)C(=O)N(C)C1CC2C1] | 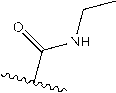 [SMILES: *C(=O)NCC] |
| 1435 | 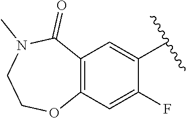 [SMILES: *c1cc2c(cc1F)OCCN(C)C2=O] | 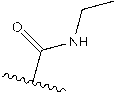 [SMILES: *C(=O)NCC] |
| 1436 | 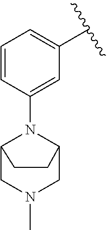 [SMILES: *c1cccc(N2C3CCC2CN(C)C3)c1] | 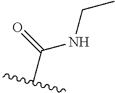 [SMILES: *C(=O)NCC] |
| 1437 | 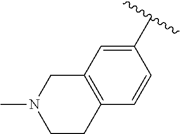 [SMILES: *c1ccc2c(c1)CN(C)CC2] | 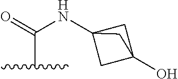 [SMILES: *C(=O)NC12CC(O)(C1)C2] |
| 1438 | 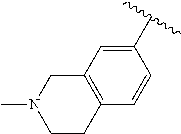 [SMILES: *c1ccc2c(c1)CN(C)CC2] | 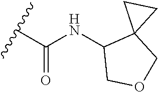 [SMILES: *C(=O)NC1COCC12CC2] |

TABLE 2a

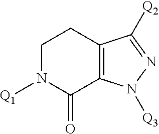

| Example | | | |
|---|---|---|---|
| Number | Q1 | Q2 | Q3 |
| 2001 | 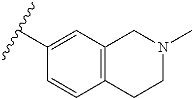 [SMILES: *c1ccc2c(c1)CN(C)CC2] | 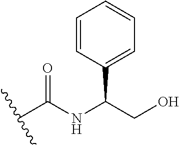 [SMILES: *C(=O)N[C@H](CO)c1ccccc1] | 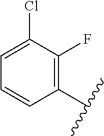 [SMILES: *c1cccc(Cl)c1F] |
| 2002 | 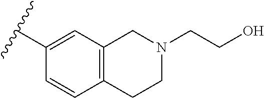 [SMILES: *c1ccc2c(c1)CN(CCO)CC2] | 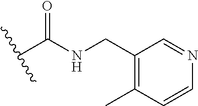 [SMILES: *C(=O)NCc1cnccc1C] | 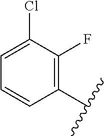 [SMILES: *c1cccc(Cl)c1F] |
| 2003 | 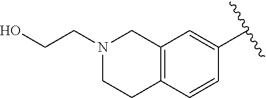 [SMILES: *c1ccc2c(c1)CN(CCO)CC2] | 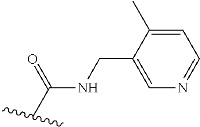 [SMILES: *C(=O)NCc1cnccc1C] | 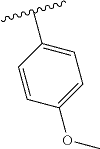 [SMILES: *c1ccc(OC)cc1] |

TABLE 2b

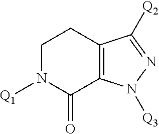

| Example | | | |
|---|---|---|---|
| Number | Q1 | Q2 | Q3 |
| 2004 | 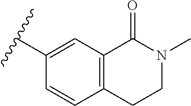 [SMILES: *c1ccc2c(c1)C(=O)N(C)CC2] | 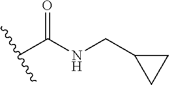 [SMILES: *C(=O)NCC1CC1] | 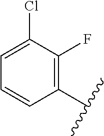 [SMILES: *c1cccc(Cl)c1F] |
| 2005 | 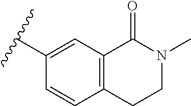 [SMILES: *c1ccc2c(c1)C(=O)N(C)CC2] | 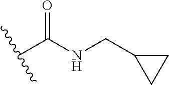 [SMILES: *C(=O)NCC1CC1] | 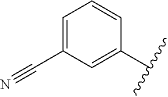 [SMILES: *c1cccc(C#N)c1] |
| 2006 | 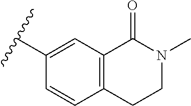 [SMILES: *c1ccc2c(c1)C(=O)N(C)CC2] | 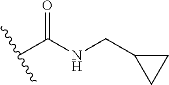 [SMILES: *C(=O)NCC1CC1] | 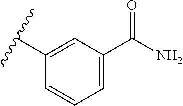 [SMILES: *c1cccc(C(N)=O)c1] |
| 2007 | 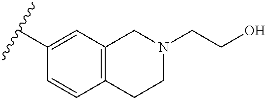 [SMILES: *c1ccc2c(c1)CN(CCO)CC2] | 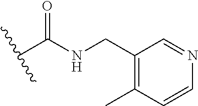 [SMILES: *C(=O)NCc1cnccc1C] | 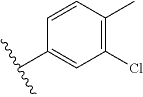 [SMILES: *c1ccc(C)c(Cl)c1] |
| 2008 | 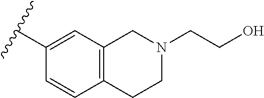 [SMILES: *c1ccc2c(c1)CN(CCO)CC2] | 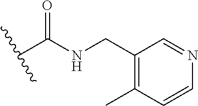 [SMILES: *C(=O)NCc1cnccc1C] | 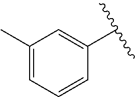 [SMILES: *c1cccc(C)c1] |
| 2009 | 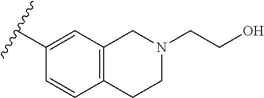 [SMILES: *c1ccc2c(c1)CN(CCO)CC2] | 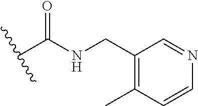 [SMILES: *C(=O)NCc1cnccc1C] | 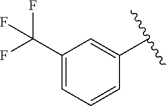 [SMILES: *c1cccc(C(F)(F)F)c1] |
| 2010 | 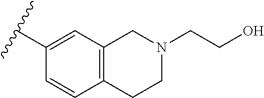 [SMILES: *c1ccc2c(c1)CN(CCO)CC2] | 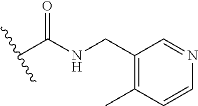 [SMILES: *C(=O)NCc1cnccc1C] | 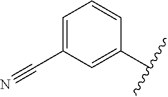 [SMILES: *c1cccc(C#N)c1] |
| 2011 | 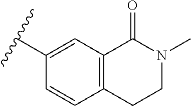 [SMILES: *c1ccc2c(c1)C(=O)N(C)CC2] | 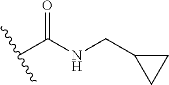 [SMILES: *C(=O)NCC1CC1] | 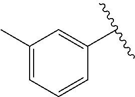 [SMILES: *c1cccc(C)c1] |
| 2012 | 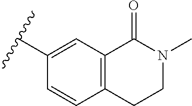 [SMILES: *c1ccc2c(c1)C(=O)N(C)CC2] | 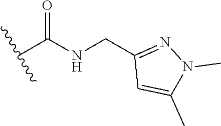 [SMILES: *C(=O)NCc1cc(C)n(C)n1] | 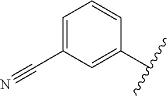 [SMILES: *c1cccc(C#N)c1] |
| 2013 | 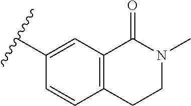 [SMILES: *c1ccc2c(c1)C(=O)N(C)CC2] | 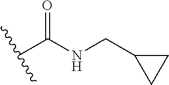 [SMILES: *C(=O)NCC1CC1] | 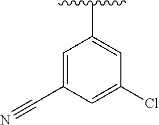 [SMILES: *c1cc(Cl)cc(C#N)c1] |
| 2014 | 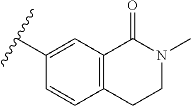 [SMILES: *c1ccc2c(c1)C(=O)N(C)CC2] | 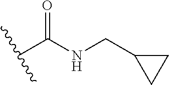 [SMILES: *C(=O)NCC1CC1] | 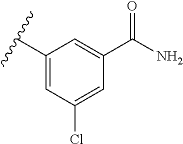 [SMILES: *c1cc(Cl)cc(C(N)=O)c1] |

TABLE 2c

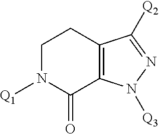

| Example | | | |
|---|---|---|---|
| Number | Q1 | Q2 | Q3 |
| 2015 | 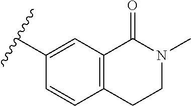 [SMILES: *c1ccc2c(c1)C(=O)N(C)CC2] | 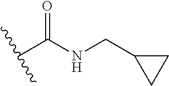 [SMILES: *C(=O)NCC1CC1] | 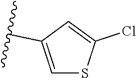 [SMILES: *c1csc(Cl)c1] |
| 2016 | 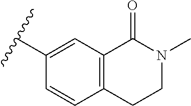 [SMILES: *c1ccc2c(c1)C(=O)N(C)CC2] | 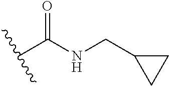 [SMILES: *C(=O)NCC1CC1] | 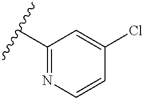 [SMILES: *c1cc(Cl)ccn1] |
| 2017 | 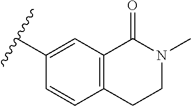 [SMILES: *c1ccc2c(c1)C(=O)N(C)CC2] | 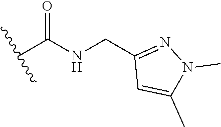 [SMILES: *C(=O)NCc1cc(C)n(C)n1] | 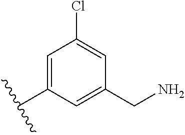 [SMILES: *c1cc(Cl)cc(CN)c1] |
| 2018 | 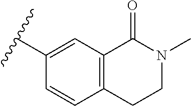 [SMILES: *c1ccc2c(c1)C(=O)N(C)CC2] | 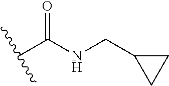 [SMILES: *C(=O)NCC1CC1] | 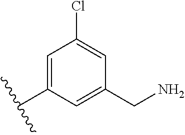 [SMILES: *c1cc(Cl)cc(CN)c1] |
| 2019 | 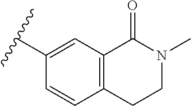 [SMILES: *c1ccc2c(c1)C(=O)N(C)CC2] | 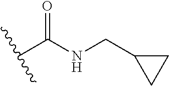 [SMILES: *C(=O)NCC1CC1] | 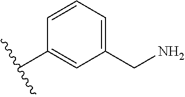 [SMILES: *c1cccc(CN)c1] |
| 2020 | 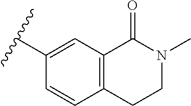 [SMILES: *c1ccc2c(c1)C(=O)N(C)CC2] | 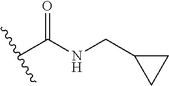 [SMILES: *C(=O)NCC1CC1] | 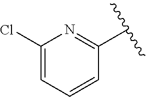 [SMILES: *c1cccc(Cl)n1] |
| 2021 | 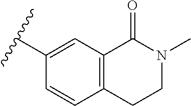 [SMILES: *c1ccc2c(c1)C(=O)N(C)CC2] | 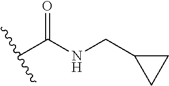 [SMILES: *C(=O)NCC1CC1] | 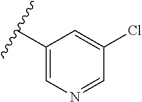 [SMILES: *c1cncc(Cl)c1] |
| 2022 | 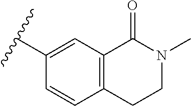 [SMILES: *c1ccc2c(c1)C(=O)N(C)CC2] | 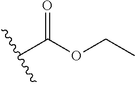 [SMILES: *C(=O)OCC] | 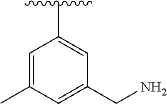 [SMILES: *c1cc(C)cc(CN)c1] |
| 2023 | 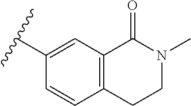 [SMILES: *c1ccc2c(c1)C(=O)N(C)CC2] | 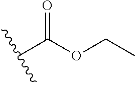 [SMILES: *C(=O)OCC] | 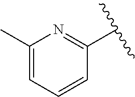 [SMILES: *c1cccc(C)n1] |
| 2024 | 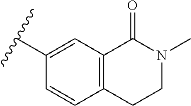 [SMILES: *c1ccc2c(c1)C(=O)N(C)CC2] | 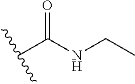 [SMILES: *C(=O)NCC] | 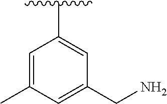 [SMILES: *c1cc(C)cc(CN)c1] |
| 2025 | 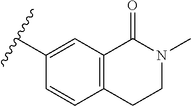 [SMILES: *c1ccc2c(c1)C(=O)N(C)CC2] | 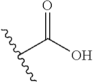 [SMILES: *C(=O)O] | 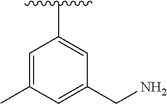 [SMILES: *c1cc(C)cc(CN)c1] |

TABLE 2d

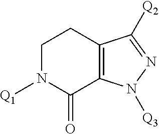

| Example | | | |
|---|---|---|---|
| Number | Q1 | Q2 | Q3 |
| 2027 | 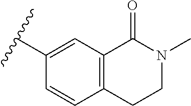 [SMILES: *c1ccc2c(c1)C(=O)N(C)CC2] | 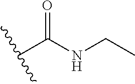 [SMILES: *C(=O)NCC] | 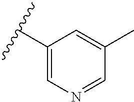 [SMILES: *c1cncc(C)c1] |
| 2028 | 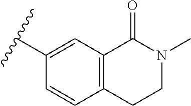 [SMILES: *c1ccc2c(c1)C(=O)N(C)CC2] | 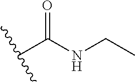 [SMILES: *C(=O)NCC] | 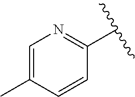 [SMILES: *c1ccc(C)cn1] |
| 2029 | 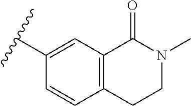 [SMILES: *c1ccc2c(c1)C(=O)N(C)CC2] | 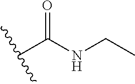 [SMILES: *C(=O)NCC] | 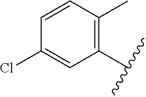 [SMILES: *c1cc(Cl)ccc1C] |
| 2030 | 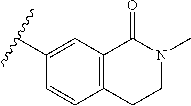 [SMILES: *c1ccc2c(c1)C(=O)N(C)CC2] | 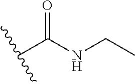 [SMILES: *C(=O)NCC] | 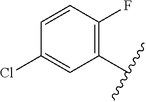 [SMILES: *c1cc(Cl)ccc1F] |
| 2031 | 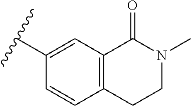 [SMILES: *c1ccc2c(c1)C(=O)N(C)CC2] | 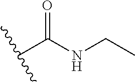 [SMILES: *C(=O)NCC] | 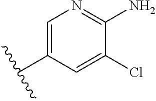 [SMILES: *c1cnc(N)c(Cl)c1] |
| 2032 | 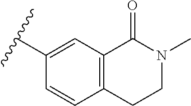 [SMILES: *c1ccc2c(c1)C(=O)N(C)CC2] | 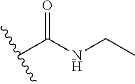 [SMILES: *C(=O)NCC] | 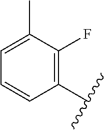 [SMILES: *c1cccc(C)c1F] |
| 2033 | 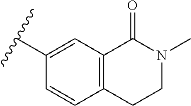 [SMILES: *c1ccc2c(c1)C(=O)N(C)CC2] | 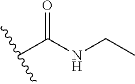 [SMILES: *C(=O)NCC] | 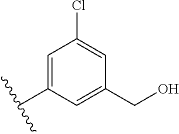 [SMILES: *c1cc(Cl)cc(CO)c1] |
| 2034 | 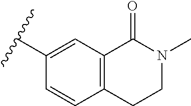 [SMILES: *c1ccc2c(c1)C(=O)N(C)CC2] | 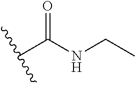 [SMILES: *C(=O)NCC] | 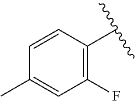 [SMILES: *c1ccc(C)cc1F] |
| 2035 | 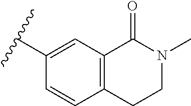 [SMILES: *c1ccc2c(c1)C(=O)N(C)CC2] | 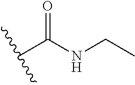 [SMILES: *C(=O)NCC] | 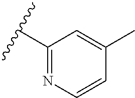 [SMILES: *c1cc(C)ccn1] |
| 2036 | 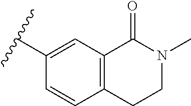 [SMILES: *c1ccc2c(c1)C(=O)N(C)CC2] | 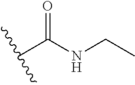 [SMILES: *C(=O)NCC] | 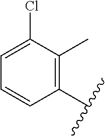 [SMILES: *c1cccc(Cl)c1C] |
| 2037 | 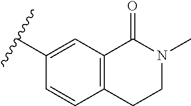 [SMILES: *c1ccc2c(c1)C(=O)N(C)CC2] | 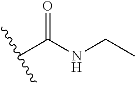 [SMILES: *C(=O)NCC] | 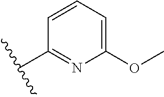 [SMILES: *c1cccc(OC)n1] |
| 2038 | 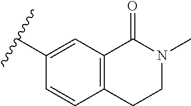 [SMILES: *c1ccc2c(c1)C(=O)N(C)CC2] | 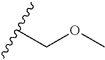 [SMILES: *COC] | 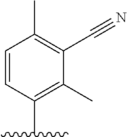 [SMILES: *c1ccc(C)c(C#N)c1C] |
| 2039 | 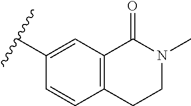 [SMILES: *c1ccc2c(c1)C(=O)N(C)CC2] | 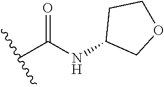 [SMILES: *C(=O)N[C@@H]1CCOC1] | 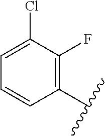 [SMILES: *c1cccc(Cl)c1F] |
| 2040 | 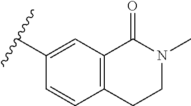 [SMILES: *c1ccc2c(c1)C(=O)N(C)CC2] | 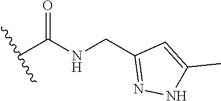 [SMILES: *C(=O)NCc1cc(C)[nH]n1] | 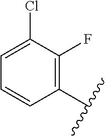 [SMILES: *c1cccc(Cl)c1F] |
| 2041 | 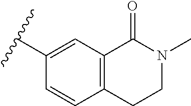 [SMILES: *c1ccc2c(c1)C(=O)N(C)CC2] | 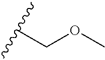 [SMILES: *COC] | 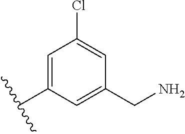 [SMILES: *c1cc(Cl)cc(CN)c1] |
| 2042 | 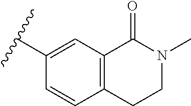 [SMILES: *c1ccc2c(c1)C(=O)N(C)CC2] | 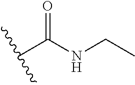 [SMILES: *C(=O)NCC] | 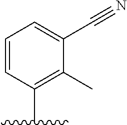 [SMILES: *c1cccc(C#N)c1C] |
| 2043 | 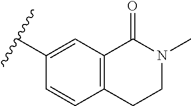 [SMILES: *c1ccc2c(c1)C(=O)N(C)CC2] | 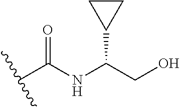 [SMILES: *C(=O)N[C@@H](CO)C1CC1] | 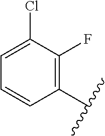 [SMILES: *c1cccc(Cl)c1F] |
| 2044 | 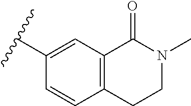 [SMILES: *c1ccc2c(c1)C(=O)N(C)CC2] | 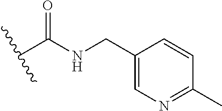 [SMILES: *C(=O)NCc1ccc(C)nc1] | 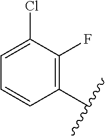 [SMILES: *c1cccc(Cl)c1F] |
| 2045 | 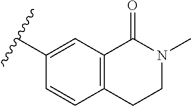 [SMILES: *c1ccc2c(c1)C(=O)N(C)CC2] | 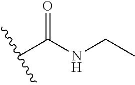 [SMILES: *C(=O)NCC] | 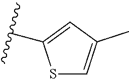 [SMILES: *c1cc(C)cs1] |
| 2046 | 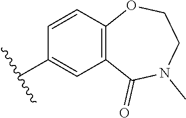 [SMILES: *c1ccc2c(c1)C(=O)N(C)CCO2] | 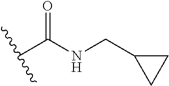 [SMILES: *C(=O)NCC1CC1] | 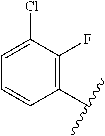 [SMILES: *c1cccc(Cl)c1F] |
| 2047 | 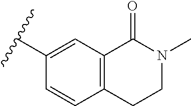 [SMILES: *c1ccc2c(c1)C(=O)N(C)CC2] | 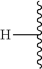 [SMILES: *[H]] | 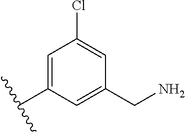 [SMILES: *c1cc(Cl)cc(CN)c1] |
| 2048 | 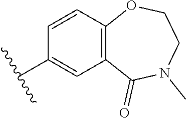 [SMILES: *c1ccc2c(c1)C(=O)N(C)CCO2] | 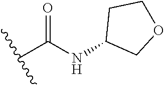 [SMILES: *C(=O)N[C@@H]1CCOC1] | 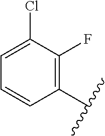 [SMILES: *c1cccc(Cl)c1F] |
| 2049 | 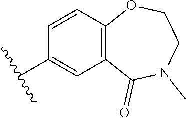 [SMILES: *c1ccc2c(c1)C(=O)N(C)CCO2] | 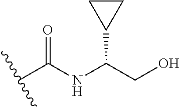 [SMILES: *C(=O)N[C@@H](CO)C1CC1] | 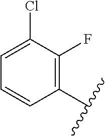 [SMILES: *c1cccc(Cl)c1F] |
| 2050 | 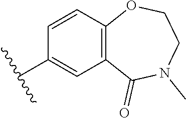 [SMILES: *c1ccc2c(c1)C(=O)N(C)CCO2] | 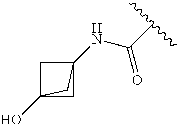 [SMILES: *C(=O)NC12CC(O)(C1)C2] | 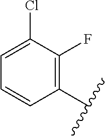 [SMILES: *c1cccc(Cl)c1F] |
| 2051 | 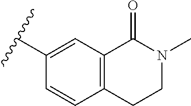 [SMILES: *c1ccc2c(c1)C(=O)N(C)CC2] | 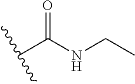 [SMILES: *C(=O)NCC] | 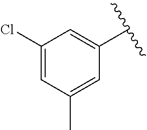 [SMILES: *c1cc(C)cc(Cl)c1] |
| 2052 | 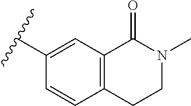 [SMILES: *c1ccc2c(c1)C(=O)N(C)CC2] | 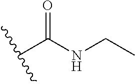 [SMILES: *C(=O)NCC] | 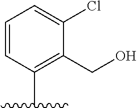 [SMILES: *c1cccc(Cl)c1CO] |
| 2053 | 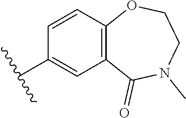 [SMILES: *c1ccc2c(c1)C(=O)N(C)CCO2] | 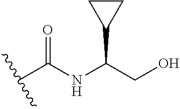 [SMILES: *C(=O)N[C@H](CO)C1CC1] | 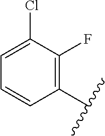 [SMILES: *c1cccc(Cl)c1F] |
| 2054 | 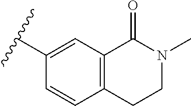 [SMILES: *c1ccc2c(c1)C(=O)N(C)CC2] | 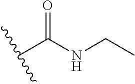 [SMILES: *C(=O)NCC] | 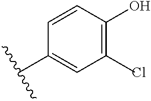 |
| 2056 | 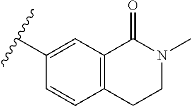 [SMILES: *c1ccc2c(c1)C(=O)N(C)CC2] | 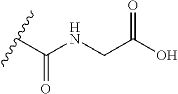 [SMILES: *C(=O)NCC(=O)O] | 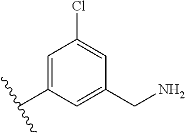 [SMILES: *c1cc(Cl)cc(CN)c1] |
| 2057 | 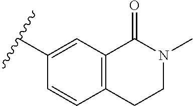 [SMILES: *c1ccc2c(c1)C(=O)N(C)CC2] | 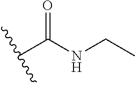 [SMILES: *C(=O)NCC] | 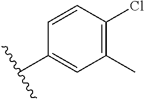 [SMILES: *c1ccc(Cl)c(C)c1] |
| 2058 | 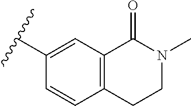 [SMILES: *c1ccc2c(c1)C(=O)N(C)CC2] | 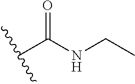 [SMILES: *C(=O)NCC] | 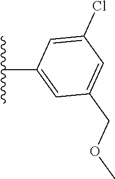 [SMILES: *c1cc(Cl)cc(COC)c1] |

TABLE 3a

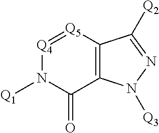

| Example | | | | | |
|---|---|---|---|---|---|
| Number | Q1 | Q2 | Q3 | Q4 | Q5 |
| 3001 | 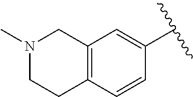 [SMILES: *c1ccc2c(c1)CN(C)CC2] | 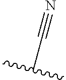 [SMILES: *C#N] | 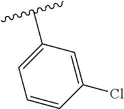 [SMILES: *c1cccc(Cl)c1] | CH | N |
| 3002 | 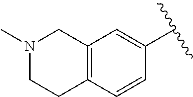 [SMILES: *c1ccc2c(c1)CN(C)CC2] | 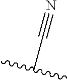 [SMILES: *C#N] | 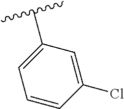 [SMILES: *c1cccc(Cl)c1] | CCH₃ | N |

TABLE 3b

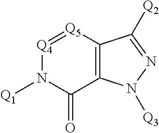

| Example | | | | | |
|---|---|---|---|---|---|
| Number | Q1 | Q2 | Q3 | Q4 | Q5 |
| 3003 | 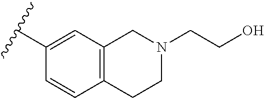 [SMILES: *c1ccc2c(c1)CN(CCO)CC2] | 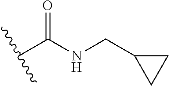 [SMILES: *C(=O)NCC1CC1] | 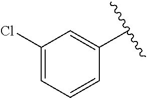 [SMILES: *c1cccc(Cl)c1] | CCH₃ | N |
| 3004 | 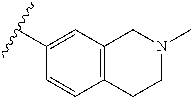 [SMILES: *c1ccc2c(c1)CN(C)CC2] | 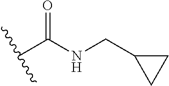 [SMILES: *C(=O)NCC1CC1] | 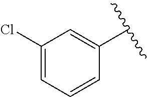 [SMILES: *c1cccc(Cl)c1] | CCH₃ | N |
| 3005 | 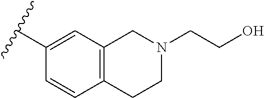 [SMILES: *c1ccc2c(c1)CN(CCO)CC2] | 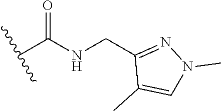 [SMILES: *C(=O)NCc1nn(C)cc1C] | 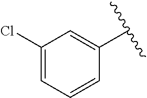 [SMILES: *c1cccc(Cl)c1] | CCH₃ | N |
| 3006 | 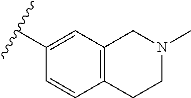 [SMILES: *c1ccc2c(c1)CN(C)CC2] | 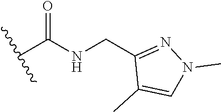 [SMILES: *C(=O)NCc1nn(C)cc1C] | 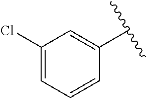 [SMILES: *c1cccc(Cl)c1] | CCH₃ | N |
| 3007 | 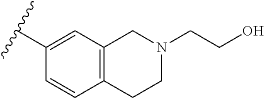 [SMILES: *c1ccc2c(c1)CN(CCO)CC2] | 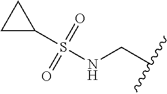 [SMILES: *CNS(=O)(=O)C1CC1] | 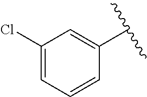 [SMILES: *c1cccc(Cl)c1] | CCH₃ | N |
| 3008 | 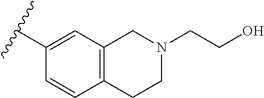 [SMILES: *c1ccc2c(c1)CN(CCO)CC2] | 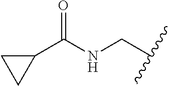 [SMILES: *CNC(=O)C1CC1] | 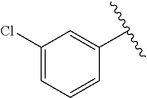 [SMILES: *c1cccc(Cl)c1] | CCH₃ | N |
| 3009 | 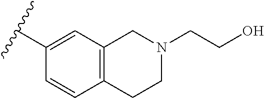 [SMILES: *c1ccc2c(c1)CN(CCO)CC2] | 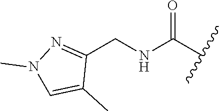 [SMILES: *C(=O)NCc1nn(C)cc1C] | 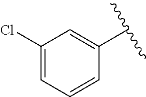 [SMILES: *c1cccc(Cl)c1] | CCH₃ | N |
| 3010 | 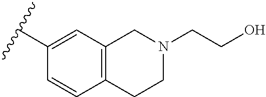 [SMILES: *c1ccc2c(c1)CN(CCO)CC2] | 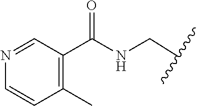 [SMILES: *CNC(=O)c1cnccc1C] | 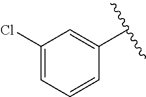 [SMILES: *c1cccc(Cl)c1] | CCH₃ | N |
| 3011 | 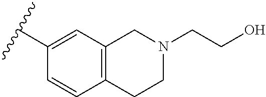 [SMILES: *c1ccc2c(c1)CN(CCO)CC2] | 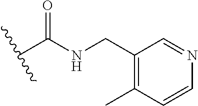 [SMILES: *C(=O)NCc1cnccc1C] | 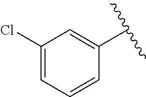 [SMILES: *c1cccc(Cl)c1] | CCH₃ | N |
| 3012 | 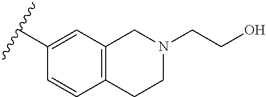 [SMILES: *c1ccc2c(c1)CN(CCO)CC2] | 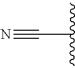 [SMILES: *C#N] | 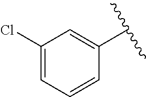 [SMILES: *c1cccc(Cl)c1] | CCH₃ | N |
| 3013 | 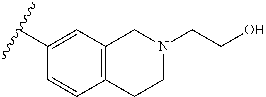 [SMILES: *c1ccc2c(c1)CN(CCO)CC2] | 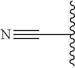 [SMILES: *C#N] | 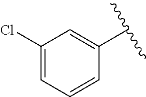 [SMILES: *c1cccc(Cl)c1] | CH | N |
| 3014 | 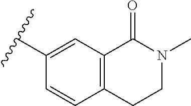 [SMILES: *c1ccc2c(c1)C(=O)N(C)CC2] | 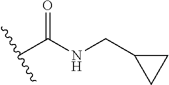 [SMILES: *C(=O)NCC1CC1] | 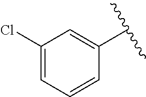 [SMILES: *c1cccc(Cl)c1] | CCH₃ | N |

TABLE 3c

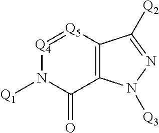

| Example | | | | | |
|---|---|---|---|---|---|
| Number | Q1 | Q2 | Q3 | Q4 | Q5 |
| 3015 | 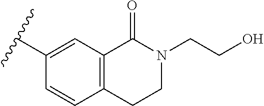 [SMILES: *c1ccc2c(c1)C(=O)N(CCO)CC2] | 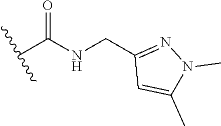 [SMILES: *C(=O)NCc1cc(C)n(C)n1] | 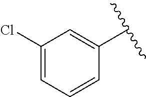 [SMILES: *c1cccc(Cl)c1] | CCH₃ | N |
| 3016 | 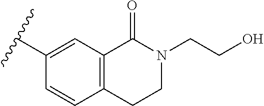 [SMILES: *c1ccc2c(c1)C(=O)N(CCO)CC2] | 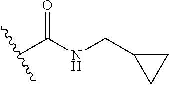 [SMILES: *C(=O)NCC1CC1] | 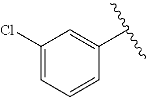 [SMILES: *c1cccc(Cl)c1] | CCH₃ | N |
| 3017 | 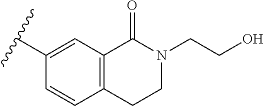 [SMILES: *c1ccc2c(c1)C(=O)N(CCO)CC2] | 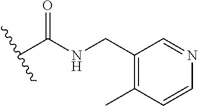 [SMILES: *C(=O)NCc1cnccc1C] | 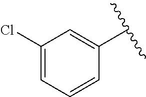 [SMILES: *c1cccc(Cl)c1] | CCH₃ | N |
| 3018 | 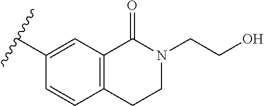 [SMILES: *c1ccc2c(c1)C(=O)N(CCO)CC2] | 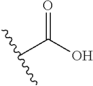 [SMILES: *C(=O)O] | 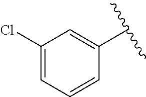 [SMILES: *c1cccc(Cl)c1] | CCH₃ | N |
| 3019 | 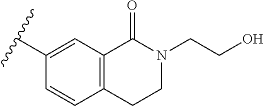 [SMILES: *c1ccc2c(c1)C(=O)N(CCO)CC2] | 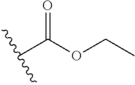 [SMILES: *C(=O)OCC] | 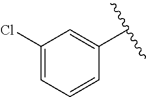 [SMILES: *c1cccc(Cl)c1] | CCH₃ | N |
| 3020 | 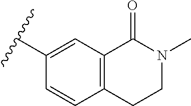 [SMILES: *c1ccc2c(c1)C(=O)N(C)CC2] | 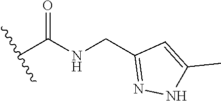 [SMILES: *C(=O)NCc1cc(C)[nH]n1] | 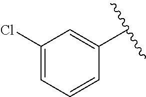 [SMILES: *c1cccc(Cl)c1] | CCH₃ | N |
| 3021 | 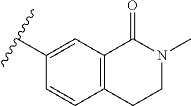 [SMILES: *c1ccc2c(c1)C(=O)N(C)CC2] | 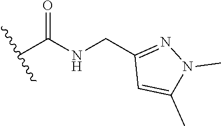 [SMILES: *C(=O)NCc1cc(C)n(C)n1] | 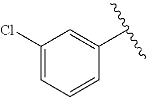 [SMILES: *c1cccc(Cl)c1] | CCH₃ | N |
| 3022 | 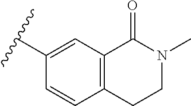 [SMILES: *c1ccc2c(c1)C(=O)N(C)CC2] | 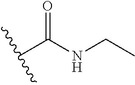 [SMILES: *C(=O)NCC] | 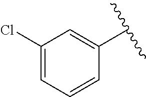 [SMILES: *c1cccc(Cl)c1] | CCH₃ | N |

TABLE 3d

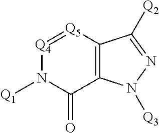

| Example | | | | | |
|---|---|---|---|---|---|
| Number | Q1 | Q2 | Q3 | Q4 | Q5 |
| 3023 | 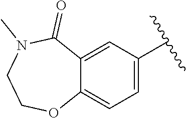 [SMILES: *c1ccc2c(c1)C(=O)N(C)CCO2] | 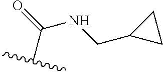 [SMILES: *C(=O)NCC1CC1] | 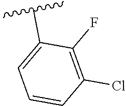 [SMILES: *c1cccc(Cl)c1F] | CCH₃ | N |

TABLE 4

| Example Number | Structure |
|---|---|
| 4001 | 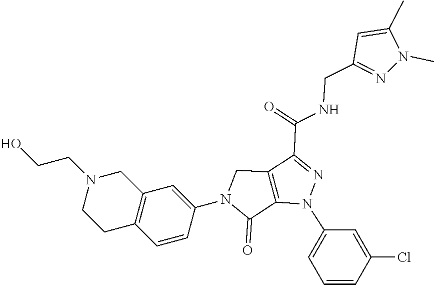 [SMILES: Cc1cc(CNC(=O)c2nn(-c3cccc(Cl)c3)c3c2CN(c2ccc4c(c2)CN(CCO)CC4)C3=O)nn1C] |
| 4002 | 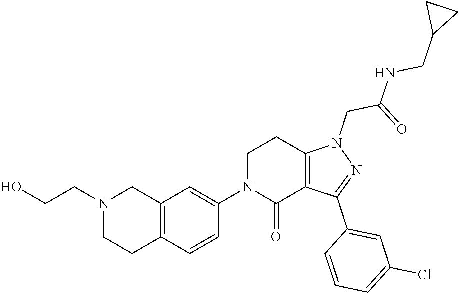 [SMILES: O=C(Cn1nc(-c2cccc(Cl)c2)c2c1CCN(c1ccc3c(c1)CN(CCO)CC3)C2=O)NCC1CC1] |
| 4003 | 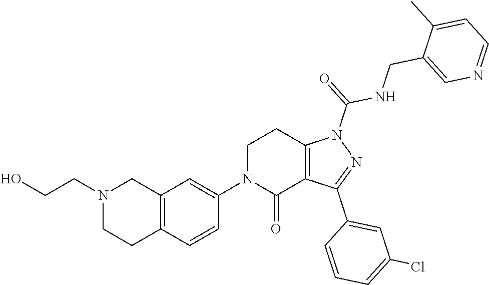 [SMILES: Cc1ccncc1CNC(=O)n1nc(-c2cccc(Cl)c2)c2c1CCN(c1ccc3c(c1)CN(CCO)CC3)C2=O] |
| 4004 | 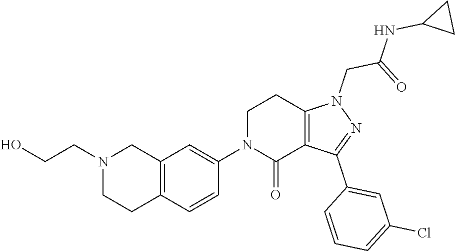 [SMILES: O=C(Cn1nc(-c2cccc(Cl)c2)c2c1CCN(c1ccc3c(c1)CN(CCO)CC3)C2=O)NC1CC1] |

Preferably, the compound of formula (I) is a compound selected from example numbers: 2017, 1001, 1002, 1003, 1005, 1006, 1007, 1008, 1010, 1012, 1013, 1014, 1015, 1017, 1019, 1020, 1021, 1022, 1023, 1024, 1025, 1026, 1027, 1028, 1029, 1030, 1031, 1032, 1034, 1035, 1036, 1037, 1038, 1039, 1041, 1042, 1043, 1044, 1045, 1046, 1047, 1048, 1049, 1050, 1051, 1052, 1053, 1054, 1055, 1056, 1057, 1058, 1059, 1060, 1061, 1063, 1064, 1065, 1068, 1069, 1070, 1075, 1076, 1077, 1078, 1079, 1080, 1081, 1082, 1083, 1084, 1085, 1086, 1087, 1088, 1089, 1090, 1091, 1092, 1093, 1096, 1098, 1099, 1101, 1102, 1103, 1104, 1105, 1106, 1107, 1108, 1109, 1110, 1111, 1112, 1113, 1115, 1116, 1117, 1118, 1119, 1120, 1121, 1122, 1123, 1124, 1125, 1126, 1127, 1128, 1129, 1130, 1131, 1132, 1133, 1134, 1135, 1136, 1137, 1138, 1139, 1140, 1141, 1142, 1143, 1144, 1145, 1146, 1147, 1148, 1149, 1150, 1151, 1152, 1153, 1154, 1155, 1156, 1157, 1158, 1159, 1160, 1161, 1162, 1163, 1164, 1166, 1167, 1168, 1169, 1170, 1171, 1172, 1173, 1174, 1175, 1176, 1177, 1178, 1179, 1180, 1181, 1185, 1186, 1187, 1188, 1189, 1191, 1193, 1194, 1197, 1198, 1199, 1200, 1201, 1202, 1203, 1204, 1209, 1210, 1211, 1213, 1214, 1215, 1216, 1217, 1218, 1219, 1220, 1221, 1222, 1223, 1224, 1225, 1226, 1227, 1229, 1230, 1231, 1233, 1234, 1235, 1236, 1237, 1238, 1239, 1240, 1245, 1246, 1247, 1248, 1249, 1250, 1251, 1252, 1253, 1254, 1255, 1256, 1257, 1258, 1259, 1260, 1261, 1262, 1264, 1265, 1266, 1267, 1268, 1271, 1274, 1275, 1280, 1282, 1283, 1284, 1287, 1288, 1289, 1290, 1292, 1293, 1294, 1296, 1297, 1299, 1302, 1303, 1304, 1305, 1306, 1307, 1308, 1309, 1310, 1314, 1317, 1318, 1319, 1320, 1321, 1325, 1326, 1327, 1328, 1329, 1331, 1332, 1333, 1334, 1336, 1337, 1338, 1339, 1340, 1341, 1342, 1344, 1346, 1347, 1352, 1353, 1355, 1357, 1359, 1360, 1367, 1370, 1371, 1372, 1373, 1374, 1376, 1377, 1378, 1379, 1380, 1382, 1383, 1384, 1388, 1391, 1393, 1394, 1396, 1404, 1406, 1408, 1410, 1411, 1412, 1414, 1415, 1416, 1417, 1418, 1421, 1424, 1425, 1427, 1429, 1433, 1435, 1437, 1438, 2001, 2002, 2003, 2004, 2005, 2007, 2008, 2009, 2010, 2011, 2012, 2014, 2018, 2019, 2020, 2022, 2023, 2024, 2025, 2032, 2033, 2039, 2040, 2041, 2043, 2044, 2046, 2048, 2049, 2050, 2051, 2053, 2056, 3001, 3002, 3003, 3004, 3005, 3006, 3007, 3008, 3009, 3010, 3011, 3012, 3013, 3014, 3015, 3016, 3017, 3019, 3020, 3021, 3022, 3023, or 4003, and pharmaceutically acceptable salts and/or solvates thereof.

Preferably, the compound of formula (I) is a compound selected from example numbers: 2017, 1005, 1021, 1022, 1037, 1051, 1055, 1065, 1077, 1078, 1079, 1089, 1099, 1107, 1109, 1110, 1111, 1125, 1126, 1127, 1128, 1129, 1130, 1132, 1134, 1135, 1136, 1137, 1138, 1139, 1140, 1141, 1144, 1146, 1147, 1150, 1154, 1155, 1157, 1158, 1159, 1160, 1161, 1162, 1163, 1166, 1168, 1170, 1171, 1172, 1177, 1189, 1217, 1219, 1220, 1221, 1222, 1230, 1234, 1236, 1254, 1256, 1275, 1288, 1299, 1305, 1306, 1317, 1318, 1319, 1321, 1352, 1376, 1377, 1378, 1382, 1383, 2001, 2002, 2008, 2018, 2022, 2024, 2025, 3005, 3006, 3011, 3015, 3017, 1001, 1007, 1013, 1015, 1019, 1023, 1024, 1030, 1031, 1035, 1044, 1049, 1052, 1053, 1054, 1059, 1060, 1061, 1063, 1068, 1070, 1075, 1076, 1080, 1083, 1084, 1085, 1086, 1087, 1088, 1090, 1091, 1092, 1093, 1098, 1103, 1105, 1106, 1108, 1112, 1117, 1118, 1121, 1122, 1123, 1131, 1133, 1142, 1143, 1145, 1148, 1151, 1156, 1165, 1167, 1169, 1173, 1174, 1175, 1178, 1186, 1188, 1193, 1199, 1200, 1201, 1203, 1204, 1209, 1210, 1213, 1215, 1216, 1218, 1223, 1224, 1225, 1226, 1227, 1229, 1231, 1233, 1235, 1237, 1238, 1239, 1245, 1247, 1248, 1253, 1255, 1258, 1259, 1262, 1271, 1289, 1290, 1293, 1294, 1296, 1307, 1309, 1314, 1320, 1325, 1326, 1327, 1355, 1371, 1374, 2004, 2007, 2009, 2010, 2019, 2020, 3002, 3003, 3004, 3012, 3013, 3014, 3016, 3019, 3020, 3021, 4003, 1002, 1003, 1006, 1008, 1010, 1012, 1014, 1017, 1020, 1025, 1026, 1027, 1028, 1029, 1032, 1034, 1036, 1038, 1039, 1041, 1042, 1043, 1045, 1046, 1047, 1048, 1050, 1056, 1057, 1058, 1064, 1069, 1081, 1082, 1096, 1101, 1102, 1104, 1113, 1115, 1116, 1119, 1120, 1124, 1149, 1152, 1153, 1164, 1176, 1179, 1180, 1181, 1185, 1187, 1191, 1194, 1197, 1198, 1202, 1211, 1214, 1240, 1246, 1249, 1250, 1251, 1252, 1257, 1260, 1261, 1264, 1265, 1266, 1267, 1268, 1274, 1280, 1282, 1283, 1284, 1287, 1292, 1297, 1302, 1303, 1304, 1308, 1310, 1328, 1329, 1331, 1332, 1333, 1334, 1336, 1337, 1338, 1339, 1340, 1341, 1342, 1344, 1346, 1347, 1353, 1357, 1359, 1360, 1367, 1370, 1372, 1373, 1379, 1380, 1384, 1388, 2003, 2005, 2011, 2012, 2014, 2023, 3001, 3007, 3008, 3009, 3010, or 3022, and pharmaceutically acceptable salts and/or solvates thereof.

Preferably, the compound of formula (I) is a compound selected from example numbers: 1005, 1021, 1022, 1037, 1051, 1055, 1065, 1077, 1078, 1079, 1089, 1099, 1107, 1109, 1110, 1111, 1125, 1126, 1127, 1128, 1129, 1130, 1132, 1134, 1135, 1136, 1137, 1138, 1139, 1140, 1141, 1144, 1146, 1147, 1150, 1154, 1155, 1157, 1158, 1159, 1160, 1161, 1162, 1163, 1166, 1168, 1170, 1171, 1172, 1177, 1189, 1217, 1219, 1220, 1221, 1222, 1230, 1234, 1236, 1254, 1256, 1275, 1288, 2001, 2002, 2008, 3005, 3006, 3011, 1001, 1007, 1013, 1015, 1019, 1023, 1024, 1030, 1031, 1035, 1044, 1049, 1052, 1053, 1054, 1059, 1060, 1061, 1063, 1068, 1070, 1075, 1076, 1080, 1083, 1084, 1085, 1086, 1087, 1088, 1090, 1091, 1092, 1093, 1098, 1103, 1105, 1106, 1108, 1112, 1117, 1118, 1121, 1122, 1123, 1131, 1133, 1142, 1143, 1145, 1148, 1151, 1156, 1165, 1167, 1169, 1173, 1174, 1175, 1178, 1186, 1188, 1193, 1199, 1200, 1201, 1203, 1204, 1209, 1210, 1213, 1215, 1216, 1218, 1223, 1224, 1225, 1226, 1227, 1229, 1231, 1233, 1235, 1237, 1238, 1239, 1245, 1247, 1248, 1253, 1255, 1258, 1259, 1262, 1271, 1289, 1290, 1293, 1294, 1296, 2004, 2007, 2009, 2010, 3002, 3003, 3004, 3012, 3013, 3014, 4003, 1002, 1003, 1006, 1008, 1010, 1012, 1014, 1017, 1020, 1025, 1026, 1027, 1028, 1029, 1032, 1034, 1036, 1038, 1039, 1041, 1042, 1043, 1045, 1046, 1047, 1048, 1050, 1056, 1057, 1058, 1064, 1069, 1081, 1082, 1096, 1101, 1102, 1104, 1113, 1115, 1116, 1119, 1120, 1124, 1149, 1152, 1153, 1164, 1176, 1179, 1180, 1181, 1185, 1187, 1191, 1194, 1197, 1198, 1202, 1211, 1214, 1240, 1246, 1249, 1250, 1251, 1252, 1257, 1260, 1261, 1264, 1265, 1266, 1267, 1268, 1274, 1280, 1282, 1283, 1284, 1287, 1292, 1297, 2003, 2005, 2011, 2012, 2014, 3001, 3007, 3008, 3009, or 3010, and pharmaceutically acceptable salts and/or solvates thereof.

Preferably, the compound of formula (I) is a compound selected from example numbers: 1005, 1021, 1022, 1037, 1051, 1055, 1065, 1077, 1078, 1079, 1089, 1099, 1107, 1109, 1110, 1111, 1125, 1126, 1127, 1128, 1129, 1130, 1132, 1134, 1135, 1136, 1137, 1138, 1139, 1140, 1141, 1144, 1146, 1147, 1150, 1154, 1155, 1157, 1158, 1159, 1160, 1161, 1162, 1163, 1166, 1168, 1170, 1171, 1172, 1177, 2001, 2002, 1001, 1007, 1013, 1015, 1019, 1023, 1024, 1030, 1031, 1035, 1044, 1049, 1052, 1053, 1054, 1059, 1060, 1061, 1063, 1068, 1070, 1075, 1076, 1080, 1083, 1084, 1085, 1086, 1087, 1088, 1090, 1091, 1092, 1093, 1098, 1103, 1105, 1106, 1108, 1112, 1117, 1118, 1121, 1122, 1123, 1131, 1133, 1142, 1143, 1145, 1148, 1151, 1156, 1165, 1167, 1169, 1173, 1174, 1175, 1178, 3002, 1002, 1003, 1006, 1008, 1010, 1012, 1014, 1017, 1020, 1025, 1026, 1027, 1028, 1029, 1032, 1034, 1036, 1038, 1039, 1041, 1042, 1043, 1045, 1046, 1047, 1048, 1050, 1056, 1057, 1058, 1064, 1069, 1081, 1082, 1096, 1101, 1102, 1104, 1113, 1115, 1116, 1119, 1120, 1124, 1149, 1152, 1153, 1164, 1176, 2003, or 3001, and pharmaceutically acceptable salts and/or solvates thereof.

More preferably, the compound of formula (I) is a compound selected from example numbers: 2017, 1001, 1005, 1007, 1013, 1015, 1019, 1021, 1022, 1023, 1024, 1030, 1031, 1035, 1037, 1044, 1049, 1051, 1052, 1053, 1054, 1055, 1059, 1060, 1061, 1063, 1065, 1068, 1070, 1075, 1076, 1077, 1078, 1079, 1080, 1083, 1084, 1085, 1086, 1087, 1088, 1089, 1090, 1091, 1092, 1093, 1098, 1099, 1103, 1105, 1106, 1107, 1108, 1109, 1110, 1111, 1112, 1117, 1118, 1121, 1122, 1123, 1125, 1126, 1127, 1128, 1129, 1130, 1131, 1132, 1133, 1134, 1135, 1136, 1137, 1138, 1139, 1140, 1141, 1142, 1143, 1144, 1145, 1146, 1147, 1148, 1150, 1151, 1154, 1155, 1156, 1157, 1158, 1159, 1160, 1161, 1162, 1163, 1166, 1167, 1168, 1169, 1170, 1171, 1172, 1173, 1174, 1175, 1177, 1178, 1186, 1188, 1189, 1193, 1199, 1200, 1201, 1203, 1204, 1209, 1210, 1213, 1215, 1216, 1217, 1218, 1219, 1220, 1221, 1222, 1223, 1224, 1225, 1226, 1227, 1229, 1230, 1231, 1233, 1234, 1235, 1236, 1237, 1238, 1239, 1245, 1247, 1248, 1253, 1254, 1255, 1256, 1258, 1259, 1262, 1271, 1275, 1288, 1289, 1290, 1293, 1294, 1296, 1299, 1305, 1306, 1307, 1309, 1314, 1317, 1318, 1319, 1320, 1321, 1325, 1326, 1327, 1352, 1355, 1371, 1374, 1376, 1377, 1378, 1382, 1383, 1393, 1394, 1404, 1406, 1414, 1415, 1416, 1424, 1435, 1437, 1438, 2001, 2002, 2004, 2007, 2008, 2009, 2010, 2018, 2019, 2020, 2022, 2024, 2025, 2039, 2040, 2041, 2043, 2044, 2046, 2048, 2049, 2050, 2053, 2056, 3002, 3003, 3004, 3005, 3006, 3011, 3012, 3013, 3014, 3015, 3016, 3017, 3019, 3020, 3021, or 4003, and pharmaceutically acceptable salts and/or solvates thereof.

More preferably, the compound of formula (I) is a compound selected from example numbers: 2017, 1005, 1021, 1022, 1037, 1051, 1055, 1065, 1077, 1078, 1079, 1089, 1099, 1107, 1109, 1110, 1111, 1125, 1126, 1127, 1128, 1129, 1130, 1132, 1134, 1135, 1136, 1137, 1138, 1139, 1140, 1141, 1144, 1146, 1147, 1150, 1154, 1155, 1157, 1158, 1159, 1160, 1161, 1162, 1163, 1166, 1168, 1170, 1171, 1172, 1177, 1189, 1217, 1219, 1220, 1221, 1222, 1230, 1234, 1236, 1254, 1256, 1275, 1288, 1299, 1305, 1306, 1317, 1318, 1319, 1321, 1352, 1376, 1377, 1378, 1382, 1383, 2001, 2002, 2008, 2018, 2022, 2024, 2025, 3005, 3006, 3011, 3015, 3017, 1001, 1007, 1013, 1015, 1019, 1023, 1024, 1030, 1031, 1035, 1044, 1049, 1052, 1053, 1054, 1059, 1060, 1061, 1063, 1068, 1070, 1075, 1076, 1080, 1083, 1084, 1085, 1086, 1087, 1088, 1090, 1091, 1092, 1093, 1098, 1103, 1105, 1106, 1108, 1112, 1117, 1118, 1121, 1122, 1123, 1131, 1133, 1142, 1143, 1145, 1148, 1151, 1156, 1165, 1167, 1169, 1173, 1174, 1175, 1178, 1186, 1188, 1193, 1199, 1200, 1201, 1203, 1204, 1209, 1210, 1213, 1215, 1216, 1218, 1223, 1224, 1225, 1226, 1227, 1229, 1231, 1233, 1235, 1237, 1238, 1239, 1245, 1247, 1248, 1253, 1255, 1258, 1259, 1262, 1271, 1289, 1290, 1293, 1294, 1296, 1307, 1309, 1314, 1320, 1325, 1326, 1327, 1355, 1371, 1374, 2004, 2007, 2009, 2010, 2019, 2020, 3002, 3003, 3004, 3012, 3013, 3014, 3016, 3019, 3020, 3021, or 4003, and pharmaceutically acceptable salts and/or solvates thereof.

More preferably, the compound of formula (I) is a compound selected from example numbers: 1005, 1021, 1022, 1037, 1051, 1055, 1065, 1077, 1078, 1079, 1089, 1099, 1107, 1109, 1110, 1111, 1125, 1126, 1127, 1128, 1129, 1130, 1132, 1134, 1135, 1136, 1137, 1138, 1139, 1140, 1141, 1144, 1146, 1147, 1150, 1154, 1155, 1157, 1158, 1159, 1160, 1161, 1162, 1163, 1166, 1168, 1170, 1171, 1172, 1177, 1189, 1217, 1219, 1220, 1221, 1222, 1230, 1234, 1236, 1254, 1256, 1275, 1288, 2001, 2002, 2008, 3005, 3006, 3011, 1001, 1007, 1013, 1015, 1019, 1023, 1024, 1030, 1031, 1035, 1044, 1049, 1052, 1053, 1054, 1059, 1060, 1061, 1063, 1068, 1070, 1075, 1076, 1080, 1083, 1084, 1085, 1086, 1087, 1088, 1090, 1091, 1092, 1093, 1098, 1103, 1105, 1106, 1108, 1112, 1117, 1118, 1121, 1122, 1123, 1131, 1133, 1142, 1143, 1145, 1148, 1151, 1156, 1165, 1167, 1169, 1173, 1174, 1175, 1178, 1186, 1188, 1193, 1199, 1200, 1201, 1203, 1204, 1209, 1210, 1213, 1215, 1216, 1218, 1223, 1224, 1225, 1226, 1227, 1229, 1231, 1233, 1235, 1237, 1238, 1239, 1245, 1247, 1248, 1253, 1255, 1258, 1259, 1262, 1271, 1289, 1290, 1293, 1294, 1296, 2004, 2007, 2009, 2010, 3002, 3003, 3004, 3012, 3013, 3014, or 4003, and pharmaceutically acceptable salts and/or solvates thereof.

More preferably, the compound of formula (I) is a compound selected from example numbers: 1005, 1021, 1022, 1037, 1051, 1055, 1065, 1077, 1078, 1079, 1089, 1099, 1107, 1109, 1110, 1111, 1125, 1126, 1127, 1128, 1129, 1130, 1132, 1134, 1135, 1136, 1137, 1138, 1139, 1140, 1141, 1144, 1146, 1147, 1150, 1154, 1155, 1157, 1158, 1159, 1160, 1161, 1162, 1163, 1166, 1168, 1170, 1171, 1172, 1177, 2001, 2002, 1001, 1007, 1013, 1015, 1019, 1023, 1024, 1030, 1031, 1035, 1044, 1049, 1052, 1053, 1054, 1059, 1060, 1061, 1063, 1068, 1070, 1075, 1076, 1080, 1083, 1084, 1085, 1086, 1087, 1088, 1090, 1091, 1092, 1093, 1098, 1103, 1105, 1106, 1108, 1112, 1117, 1118, 1121, 1122, 1123, 1131, 1133, 1142, 1143, 1145, 1148, 1151, 1156, 1165, 1167, 1169, 1173, 1174, 1175, 1178, or 3002, and pharmaceutically acceptable salts and/or solvates thereof.

More preferably, the compound of formula (I) is a compound selected from example numbers: 2017, 1005, 1021, 1022, 1037, 1051, 1055, 1065, 1077, 1078, 1079, 1089, 1099, 1107, 1109, 1110, 1111, 1118, 1125, 1126, 1127, 1128, 1129, 1130, 1132, 1134, 1135, 1136, 1137, 1138, 1139, 1140, 1141, 1144, 1146, 1147, 1150, 1154, 1155, 1157, 1158, 1159, 1160, 1161, 1162, 1163, 1166, 1168, 1170, 1171, 1172, 1177, 1189, 1217, 1219, 1220, 1221, 1222, 1230, 1234, 1236, 1254, 1256, 1275, 1288, 1299, 1305, 1306, 1317, 1318, 1319, 1321, 1352, 1376, 1377, 1378, 1382, 1383, 1393, 1394, 2001, 2002, 2008, 2018, 2022, 2024, 2025, 2040, 2041, 2043, 2044, 2046, 2048, 2049, 2050, 2053, 2056, 3005, 3006, 3011, 3015, or 3017, and pharmaceutically acceptable salts and/or solvates thereof.

More preferably, the compound of formula (I) is a compound selected from example numbers: 2017, 1005, 1021, 1022, 1037, 1051, 1055, 1065, 1077, 1078, 1079, 1089, 1099, 1107, 1109, 1110, 1111, 1118, 1125, 1126, 1127, 1128, 1129, 1130, 1132, 1134, 1135, 1136, 1137, 1138, 1139, 1140, 1141, 1144, 1146, 1147, 1150, 1154, 1155, 1157, 1158, 1159, 1160, 1161, 1162, 1163, 1166, 1168, 1170, 1171, 1172, 1177, 1189, 1217, 1219, 1220, 1221, 1222, 1230, 1234, 1236, 1254, 1256, 1275, 1288, 1299, 1305, 1306, 1317, 1318, 1319, 1321, 1352, 1376, 1377, 1378, 1382, 1383, 2001, 2002, 2008, 2018, 2022, 2024, 2025, 3005, 3006, 3011, 3015, or 3017, and pharmaceutically acceptable salts and/or solvates thereof.

More preferably, the compound of formula (I) is a compound selected from example numbers: 1005, 1021, 1022, 1037, 1051, 1055, 1065, 1077, 1078, 1079, 1089, 1099, 1107, 1109, 1110, 1111, 1118, 1125, 1126, 1127, 1128, 1129, 1130, 1132, 1134, 1135, 1136, 1137, 1138, 1139, 1140, 1141, 1144, 1146, 1147, 1150, 1154, 1155, 1157, 1158, 1159, 1160, 1161, 1162, 1163, 1166, 1168, 1170, 1171, 1172, 1177, 1189, 1217, 1219, 1220, 1221, 1222, 1230, 1234, 1236, 1254, 1256, 1275, 1288, 2001, 2002, 2008, 3005, 3006, or 3011, and pharmaceutically acceptable salts and/or solvates thereof.

More preferably, the compound of formula (I) is a compound selected from example numbers: 1005, 1021, 1022, 1037, 1051, 1055, 1065, 1077, 1078, 1079, 1089, 1099, 1107, 1109, 1110, 1111, 1118, 1125, 1126, 1127, 1128, 1129, 1130, 1132, 1134, 1135, 1136, 1137, 1138, 1139, 1140, 1141, 1144, 1146, 1147, 1150, 1154, 1155, 1157, 1158, 1159, 1160, 1161, 1162, 1163, 1166, 1168, 1170, 1171, 1172, 1177, 2001, 1024, or 2002, and pharmaceutically acceptable salts and/or solvates thereof.

More preferably, the compound of formula (I) is a compound selected from example numbers: 2017, 1005, 1021, 1022, 1037, 1051, 1055, 1065, 1077, 1078, 1079, 1089, 1099, 1107, 1109, 1110, 1111, 1125, 1126, 1127, 1128, 1129, 1130, 1132, 1134, 1135, 1136, 1137, 1138, 1139, 1140, 1141, 1144, 1146, 1147, 1150, 1154, 1155, 1157, 1158, 1159, 1160, 1161, 1162, 1163, 1166, 1168, 1170, 1171, 1172, 1177, 1189, 1217, 1219, 1220, 1221, 1222, 1230, 1234, 1236, 1254, 1256, 1275, 1288, 1299, 1305, 1306, 1317, 1318, 1319, 1321, 1352, 1376, 1377, 1378, 1382, 1383, 1393, 1394, 2001, 2002, 2008, 2018, 2022, 2024, 2025, 2040, 2041, 2043, 2044, 2046, 2048, 2049, 2050, 2053, 2056, 3005, 3006, 3011, 3015, or 3017, and pharmaceutically acceptable salts and/or solvates thereof.

More preferably, the compound of formula (I) is a compound selected from example numbers: 2017, 1005, 1021, 1022, 1037, 1051, 1055, 1065, 1077, 1078, 1079, 1089, 1099, 1107, 1109, 1110, 1111, 1125, 1126, 1127, 1128, 1129, 1130, 1132, 1134, 1135, 1136, 1137, 1138, 1139, 1140, 1141, 1144, 1146, 1147, 1150, 1154, 1155, 1157, 1158, 1159, 1160, 1161, 1162, 1163, 1166, 1168, 1170, 1171, 1172, 1177, 1189, 1217, 1219, 1220, 1221, 1222, 1230, 1234, 1236, 1254, 1256, 1275, 1288, 1299, 1305, 1306, 1317, 1318, 1319, 1321, 1352, 1376, 1377, 1378, 1382, 1383, 2001, 2002, 2008, 2018, 2022, 2024, 2025, 3005, 3006, 3011, 3015, or 3017, and pharmaceutically acceptable salts and/or solvates thereof.

More preferably, the compound of formula (I) is a compound selected from example numbers: 1005, 1021, 1022, 1037, 1051, 1055, 1065, 1077, 1078, 1079, 1089, 1099, 1107, 1109, 1110, 1111, 1125, 1126, 1127, 1128, 1129, 1130, 1132, 1134, 1135, 1136, 1137, 1138, 1139, 1140, 1141, 1144, 1146, 1147, 1150, 1154, 1155, 1157, 1158, 1159, 1160, 1161, 1162, 1163, 1166, 1168, 1170, 1171, 1172, 1177, 1189, 1217, 1219, 1220, 1221, 1222, 1230, 1234, 1236, 1254, 1256, 1275, 1288, 2001, 2002, 2008, 3005, 3006, or 3011, and pharmaceutically acceptable salts and/or solvates thereof.

More preferably, the compound of formula (I) is a compound selected from example numbers: 1005, 1021, 1022, 1037, 1051, 1055, 1065, 1077, 1078, 1079, 1089, 1099, 1107, 1109, 1110, 1111, 1125, 1126, 1127, 1128, 1129, 1130, 1132, 1134, 1135, 1136, 1137, 1138, 1139, 1140, 1141, 1144, 1146, 1147, 1150, 1154, 1155, 1157, 1158, 1159, 1160, 1161, 1162, 1163, 1166, 1168, 1170, 1171, 1172, 1177, 2001, 1024, or 2002, and pharmaceutically acceptable salts and/or solvates thereof.

Even more preferably, the compound of formula (I) is a compound selected from example numbers: 1024, 1055, 1128, 1118, 1166, 1222, 1236, 2039, 2048, or 2049, and pharmaceutically acceptable salts and/or solvates thereof.

Even more preferably, the compound of formula (I) is a compound selected from example numbers: 1024, 1055, 1128, 1118, 1166, 1222, or 1236, and pharmaceutically acceptable salts and/or solvates thereof.

Even more preferably, the compound of formula (I) is a compound selected from example numbers: 1024, 1055, 1128, 1118, or 1166, and pharmaceutically acceptable salts and/or solvates thereof.

Most preferably, the compound of formula (I) is a compound selected from example numbers: 1024, 1055, 1128, 1149, 1220, 1222, 2039, 2048, or 2049, and pharmaceutically acceptable salts and/or solvates thereof.

Most preferably, the compound of formula (I) is a compound selected from example numbers: 1024, 1055, 1128, 1149, 1220, or 1222, and pharmaceutically acceptable salts and/or solvates thereof.

Most preferably, the compound of formula (I) is a compound selected from example numbers: 1024, 1055, 1128, 1149, or 1220, and pharmaceutically acceptable salts and/or solvates thereof.

Most preferably, the compound of formula (I) is a compound selected from example numbers: 1024, 1055, or 1128, and pharmaceutically acceptable salts and/or solvates thereof.

Yet more preferably, the compound of formula (I) is a compound selected from example numbers: 2039, 2048, or 2049, and pharmaceutically acceptable salts and/or solvates thereof.

### Therapeutic Applications

As noted above, the compounds (or pharmaceutically acceptable salts and/or solvates thereof), and pharmaceutical compositions comprising the compounds (or pharmaceutically acceptable salts and/or solvates thereof) of the present invention are inhibitors of FXIIa. They are therefore useful in the treatment of disease conditions for which FXIIa is a causative factor.
Accordingly, the present invention provides a compound of the invention (or a pharmaceutically acceptable salt and/or solvate thereof), or a pharmaceutical composition comprising a compound of the invention (or a pharmaceutically acceptable salt and/or solvate thereof), for use in medicine.

The present invention also provides for the use of a compound of the invention (or a pharmaceutically acceptable salt and/or solvate thereof), or a pharmaceutical composition comprising the compound of the invention (or a pharmaceutically acceptable salt and/or solvate thereof), in the manufacture of a medicament for the treatment or prevention of a disease or condition in which FXIIa activity is implicated.

The present invention also provides a method of treatment of a disease or condition in which FXIIa activity is implicated comprising administration to a subject in need thereof a therapeutically effective amount of a compound of the invention (or a pharmaceutically acceptable salt and/or solvate thereof), or a pharmaceutical composition comprising the compound of the invention (or a pharmaceutically acceptable salt and/or solvate thereof).
As discussed above, FXIIa can mediate the conversion of plasma kallikrein from plasma prekallikrein. Plasma kallikrein can then cause the cleavage of high molecular weight kininogen to generate bradykinin, which is a potent inflammatory hormone. Inhibiting FXIIa has the potential to inhibit (or even prevent) plasma kallikrein production. Thus, the disease or condition in which FXIIa activity is implicated can be a bradykinin-mediated angioedema.

The bradykinin-mediated angioedema can be non-hereditary. For example, the non-hereditary bradykinin-mediated angioedema can be selected from non-hereditary angioedema with normal C1 Inhibitor (AE-nC1 Inh), which can be environmental, hormonal, or drug-induced; acquired angioedema; anaphylaxis associated angioedema; angiotensin converting enzyme (ACE or ace) inhibitor-induced angioedema; dipeptidyl peptidase-4 inhibitor-induced angioedema; and tPA-induced angioedema (tissue plasminogen activator-induced angioedema).
Alternatively, and preferably, the bradykinin-mediated angioedema can be hereditary angioedema (HAE), which is angioedema caused by an inherited dysfunction/fault/mutation. Types of HAE that can be treated with compounds according to the invention include HAE type 1, HAE type 2, and normal C1 inhibitor HAE (normal C1 Inh HAE).

The disease or condition in which FXIIa activity is implicated can be selected from vascular hyperpermeability, stroke including ischemic stroke and haemorrhagic accidents; retinal edema; diabetic retinopathy; impaired visual acuity; DME; retinal vein occlusion; and AMD. These conditions can also be bradykinin-mediated.
As discussed above, FXIIa can activate FXIa to cause a coagulation cascade. Thrombotic disorders are linked to this cascade. Thus, the disease or condition in which FXIIa activity is implicated can be a thrombotic disorder. More specifically, the thrombotic disorder can be thrombosis; thromboembolism caused by increased propensity of medical devices that come into contact with blood to clot blood; prothrombotic conditions such as disseminated intravascular coagulation (DIC), Venous thromboembolism (VTE), cancer associated thrombosis, complications caused by mechanical and bioprosthetic heart valves, complications caused by catheters, complications caused by ECMO, complications caused by LVAD, complications caused by dialysis, complications caused by CPB, sickle cell disease, joint arthroplasty, thrombosis induced to tPA, Paget-Schroetter syndrome and Budd-Chari syndrome; atherosclerosis; COVID-19; COVID-19 associated with SARS-COV-2 infection; acute respiratory distress syndrome (ARDS); idiopathic pulmonary fibrosis (IPF); rheumatoid arthritis (RA); and cold-induced urticarial autoinflammatory syndrome. The disease or condition in which FXIIa activity is implicated can be hereditary angioedema; COVID-19; COVID-19 associated with SARS-COV-2 infection; and idiopathic pulmonary fibrosis (IPF). The disease or condition in which FXIIa activity is implicated can be COVID-19. The disease or condition in which FXIIa activity is implicated can be COVID-19 associated with SARS-COV-2 Infection. The disease or condition in which FXIIa activity is implicated can be idiopathic pulmonary fibrosis (IPF).

Surfaces of medical devices that come into contact with blood can cause thrombosis. The compounds (or pharmaceutically acceptable salts and/or solvates thereof) and pharmaceutical compositions of the present invention can be coated on the surfaces of devices that come into contact with blood to mitigate the risk of the device causing thrombosis. For instance, they can lower the propensity these devices to clot blood and therefore cause thrombosis. Examples of devices that come into contact with blood include vascular grafts, stents, in dwelling catheters, external catheters, orthopedic prosthesis, cardiac prosthesis, and extracorporeal circulation systems.

Other disease conditions for which FXIIa is a causative factor include: neuroinflammation; neuroinflammatory/neurodegenerative disorders such as MS (multiple sclerosis); other neurodegenerative diseases such as Alzheimer's disease, epilepsy and migraine; sepsis; bacterial sepsis; inflammation; vascular hyperpermeability; and anaphylaxis.

Brown adipose tissue (BAT) thermogenic activity can be mediated by the kallikrein-kinin system, and impaired BAT activity is associated with obesity and insulin resistance. Inhibiting FXIIa has the potential to inhibit (or even prevent) BAT activity mediated by the kallikrein-kinin system. The compounds (or pharmaceutically acceptable salts and/or solvates thereof) and pharmaceutical compositions of the invention can therefore treat disease conditions such as obesity and diabetes.

Factor XII inhibition is further implicated in the treatment of disease conditions such as kidney disease, renal fibrosis, glomerulosclerosis, renal scarring, ischemia/reperfusion injury in native or transplant kidneys, and acute kidney injury.

Brain glioblastoma growth and malignancy, as well as complications thereof including oedema and cognitive function, may be beneficially affected by modulation of B1 and B2 kinin receptors, part of the kallikrein-kinin system, which are activated by bradykinin. Therefore, the disease or condition in which

FXIIa activity is implicated can be brain glioblastoma growth and malignancy, as well as complications thereof including oedema and cognitive function.

Blocking the effect of bradykinin or inhibiting the activation of the kallikrein-kinin system has a therapeutic effect in treating and preventing the occurrence of intradialytic hypotension (IDH). Therefore, the disease or condition in which FXIIa activity is implicated can be intradialytic hypotension (IDH).

### Combination Therapy

The compounds of the present invention (or pharmaceutically acceptable salts and/or solvates thereof) may be administered in combination with other therapeutic agents. Suitable combination therapies include any compound of the present invention (or a pharmaceutically acceptable salt and/or solvate thereof) combined with one or more agents selected from agents that inhibit platelet-derived growth factor (PDGF), vascular endothelial growth factor (VEGF), integrin alpha5beta1, steroids, other agents that inhibit FXIIa and other inhibitors of inflammation.

Some specific examples of therapeutic agents that may be combined with the compounds of the present invention include those disclosed in EP2281885A₁ and by S. Patel in Retina, 2009 June; 29 (6 Suppl): S45-8.

Other suitable combination therapies include a compound of the invention (or a pharmaceutically acceptable salt and/or solvate thereof) combined with one or more agents selected from agents that treat HAE (as defined generally herein), for example bradykinin B2 antagonists such icatibant (Firazyr®); plasma kallikrein inhibitors such as ecallantide (Kalbitor®), lanadelumab (Takhzyro®) and berotralstat (ORLADEYO™); or C1 esterase inhibitor such as Cinryze® and Haegarda® and Berinert® and Ruconest®.

Other suitable combination therapies include a compound of the invention (or a pharmaceutically acceptable salt and/or solvate thereof) combined with one or more agents selected from agents that are antithrombotics (as outlined above), for example other Factor XIIa inhibitors, thrombin receptor antagonists, thrombin inhibitors, factor VIIa inhibitors, factor Xa inhibitors, factor XIa inhibitors, factor IXa inhibitors, adenosine diphosphate antiplatelet agents (e.g., P2Y12 antagonists), fibrinogen receptor antagonists (e.g. to treat or prevent unstable angina or to prevent reocclusion after angioplasty and restenosis), aspirin and platelet aggregation inhibitors.

When combination therapy is employed, the compounds of the present invention and said combination agents may exist in the same or different pharmaceutical compositions, and may be administered separately, sequentially or simultaneously.

The compounds of the present invention can be administered in combination with laser treatment of the retina. The combination of laser therapy with intravitreal injection of an inhibitor of VEGF for the treatment of diabetic macular edema is known (Elman M, Aiello L, Beck R, et al. “Randomized trial evaluating ranibizumab plus prompt or deferred laser or triamcinolone plus prompt laser for diabetic macular edema” Ophthalmology. 27 Apr. 2010).

### Definitions

As noted above, the term “alkyl” is a linear saturated hydrocarbon having up to 10 carbon atoms (C₁-C₁₀) or a branched saturated hydrocarbon of between 3 and 10 carbon atoms (C₃-C₁₀); alkyl may optionally be substituted with 1, 2 or 3 substituents independently selected from (C₁-C₆)alkoxy, OH, —NR₁₂R₁₃, —C(═O)NR₁₂R₁₃, CN, CF₃, and halo. Examples of such alkyl groups include, but are not limited, to C₁-methyl, C₂-ethyl, C₃-propyl and C₄-n-butyl, C₃-iso-propyl, C₄-sec-butyl, C₄-iso-butyl, CL-tert-butyl and C₅-neo-pentyl, optionally substituted as noted above. More specifically, “alkyl” can be a linear saturated hydrocarbon having up to 6 carbon atoms (C₁-C₆) or a branched saturated hydrocarbon of between 3 and 6 carbon atoms (C₃-C₆), optionally substituted as noted above. Even more specifically, “alkyl” can be a linear saturated hydrocarbon having up to 4 carbon atoms (C₁-C₄) or a branched saturated hydrocarbon of between 3 and 4 carbon atoms (C₃-C₄), optionally substituted with 1 or 2 substituents independently selected from cyclopropyl, OH, CN, CF₃, NH₂, —C(═O)NH₂, and halo, which is herein called “small alkyl”.

Preferably, “alkyl” can be defined as a “small alkyl”. More specifically, “small alkyl” can be “small alkyl^{a}”, which is herein a linear saturated hydrocarbon having up to 4 carbon atoms (C₁-C₄) or a branched saturated hydrocarbon of between 3 and 4 carbon atoms (C₃-C₄); small alkyl^{a} may optionally be substituted with 1 or 2 substituents independently selected from cyclopropyl, OH, CN, CF₃, —C(═O)NH₂, and halo. Alternatively, “alkyl” can be defined as “small alkyl^{b}”, wherein small alkyl^{b} is a linear saturated hydrocarbon having up to 4 carbon atoms (C₁-C₄) or a branched saturated hydrocarbon of between 3 and 4 carbon atoms (C₃-C₄), optionally substituted with 1 or 2 substituents independently selected from cyclopropyl, OH, OMe (O—CH₃), OEt (O—CH₂CH₃), OⁱPr (O—CH(CH₃) 2), OⁱPr (O—CH₂CH₂CH₃), O(cyclopropyl), OCF₃, OCHF₂, CN, CF₃, NH₂, —C(═O)NH₂, and halo. More specifically, “small alkyl^{b}” can be “small alkyl” ”, which is herein a linear saturated hydrocarbon having up to 4 carbon atoms (C₁-C₄) or a branched saturated hydrocarbon of between 3 and 4 carbon atoms (C₃-C₄); small alkyl^{c} may optionally be substituted with 1 or 2 substituents independently selected from cyclopropyl, OH, OMe, OEt, CN, CF₃, —C(═O)NH₂, and halo.
As noted above, “aryl” is phenyl, biphenyl or naphthyl; aryl may be optionally substituted with 1, 2 or 3 substituents independently selected from alkyl, alkoxy, methylenedioxy, ethylenedioxy, OH, halo, CN, —(CH₂)₀₋₃—NR₁₂R₁₃, OCF₃ and CF₃. Examples of suitable aryl groups include phenyl, biphenyl and naphthyl (each optionally substituted as previously stated). Preferably “aryl” is selected from phenyl, substituted phenyl (wherein said substituents are selected from those stated above) and naphthyl. Most preferably “aryl” is selected from phenyl and substituted phenyl (wherein said substituents are selected from those stated above).
As noted above, the term “cycloalkyl^{b}” is a monocyclic saturated hydrocarbon ring of between 3 and 6 carbon atoms (C₃-C₆); cycloalkyl may optionally be substituted with 1 or 2 substituents independently selected from alkyl, (C₁-C₆) alkoxy, OH, NH₂, NH(small alkyl), CN, CF₃, halo, and —(C═O)—OH. Examples of suitable monocyclic cycloalkyl groups include cyclopropyl, cyclobutyl, cyclopentyl, and cyclohexyl, optionally substituted as noted above. More specifically, “cycloalkyl^{b}” can be a monocyclic saturated hydrocarbon ring of between 3 and 5 carbon atoms, more specifically, between 3 and 4 carbon atoms, optionally substituted as noted above.
As noted above, the term “cycloalkyl^{a}” is a monocyclic saturated hydrocarbon ring of between 3 and 6 carbon atoms (C₃-C₆); cycloalkyl may optionally be substituted with 1 or 2 substituents independently selected from alkyl, (C₁-C₆) alkoxy, OH, CN, CF₃, halo, and —(C═O)—OH. Examples of suitable monocyclic cycloalkyl groups include cyclopropyl, cyclobutyl, cyclopentyl, and cyclohexyl, optionally substituted as noted above. More specifically, “cycloalkyl^{a}” can be a monocyclic saturated hydrocarbon ring of between 3 and 5 carbon atoms, more specifically, between 3 and 4 carbon atoms, optionally substituted as noted above.
As noted above, the term “cycloalkyl” is a monocyclic saturated hydrocarbon ring of between 3 and 6 carbon atoms (C₃-C₆); cycloalkyl may optionally be substituted with 1 or 2 substituents independently selected from alkyl, (C₁-C₆) alkoxy, OH, CN, CF₃, and halo. Examples of suitable monocyclic cycloalkyl groups include cyclopropyl, cyclobutyl, cyclopentyl, and cyclohexyl, optionally substituted as noted above. More specifically, “cycloalkyl” can be a monocyclic saturated hydrocarbon ring of between 3 and 5 carbon atoms, more specifically, between 3 and 4 carbon atoms, optionally substituted as noted above.
As noted above, “polycycloalkyl” is a bicyclic saturated hydrocarbon ring system of between 5 and 8 carbon atoms (C₅-C₈), wherein the bicyclic ring is bridged, or is adamantyl; polycycloalkyl may optionally be substituted with 1 or 2 substituents independently selected from alkyl, (C₁-C₆) alkoxy, OH, CN, CF₃, and halo. Examples of suitable bridged bicyclic rings include bicyclo[1.1.1]pentane, bicyclo[2.1.1]hexane, bicyclo[2.2.1]heptane, and bicyclo[2.2.2]octane, optionally substituted as noted above. More specifically, “polycycloalkyl” can be a bridged bicyclic saturated hydrocarbon ring system of between 5 and 6 carbon atoms or adamantyl, more specifically, a bridged bicyclic saturated hydrocarbon ring system of between and 6 carbon atoms, optionally substituted as noted above.
As noted above, the term “alkoxy” is —OCF₃, O(cyclopropyl), a linear O-linked hydrocarbon of between 1 and 6 carbon atoms (C₁-C₆), or a branched O-linked hydrocarbon of between 3 and 6 carbon atoms (C₃-C₆); alkoxy may optionally be substituted, where possible, with 1 or 2 substituents independently selected from cyclopropyl, OH, CN, CF₃, and halo. Examples of such alkoxy groups include, but are not limited to, C₁-methoxy, C₂-ethoxy, C₃-n-propoxy and C₄-n-butoxy for linear alkoxy, and C₃-iso-propoxy, and C₄-sec-butoxy and tert-butoxy for branched alkoxy, optionally substituted as noted above. More specifically, “alkoxy” can be —OCF₃, O(cyclopropyl), a linear O-linked hydrocarbon of between 1 and 4 10 carbon atoms (C₁-C₄), or a branched O-linked hydrocarbon of between 3 and 4 carbon atoms (C₃-C₄); and may be optionally be substituted with 1 or 2 substituents independently selected from cyclopropyl, OH, CN, CF₃, and halo. This is herein called “small alkoxy”. Alternatively, the term “alkoxy” can be —OCF₃, a linear O-linked hydrocarbon of between 1 and 6 carbon atoms (C₁-C₆), or a branched O-linked hydrocarbon of between 3 and 6 carbon atoms (C₃-C₆); alkoxy may optionally be substituted, where possible, with 1 or 2 substituents independently selected from cyclopropyl, OH, CN, CF₃, and halo. Alternatively and more specifically, “alkoxy” can be —OCF₃, a linear O-linked hydrocarbon of between 1 and 4 carbon atoms (C₁-C₄), or a branched O-linked hydrocarbon of between 3 and 4 carbon atoms (C₃-C₄); and may be optionally be substituted with 1 or 2 substituents independently selected from cyclopropyl, OH, CN, CF₃, and halo, also herein called “small alkoxy”. Preferably, “alkoxy” can be defined as a “small alkoxy”.
As noted above, “halo” is CI, F, Br or I. More specifically, halo can be selected from C₁ and F.
As noted above, “heteroaryl” is a 5- or 6-membered carbon-containing aromatic ring containing one, two or three ring members that are selected from N, S, and O; heteroaryl may be optionally substituted with 1, 2 or 3 substituents independently selected from alkyl, alkoxy, cyclopropyl, OH, halo, CN, and CF₃. In some embodiments, “heteroaryl” can be a 5- or 6-membered carbon-containing aromatic ring containing one, two or three ring members that are selected from N, S, and O; heteroaryl may be optionally substituted with 1, 2 or 3 substituents independently selected from alkyl, alkoxy, OH, halo, CN, and CF₃. For example, heteroaryl can be selected from thiophene, furan, pyrrole, pyrazole, imidazole, oxazole, isoxazole, thiazole, isothiazole, triazole, oxadiazole, thiadiazole, pyridine, pyridazine, pyrimidine, and pyrazine, optionally substituted as noted above.
As noted above, heteroaryl^{a} is a 5-, 6-, 9- or 10-membered mono- or bi-cyclic aromatic ring containing, where possible, 1, 2, 3 or 4 ring members independently selected from N, S, and O; wherein heteroaryl^{a} may be optionally substituted with 1, 2 or 3 substituents independently selected from alkyl, cyclopropyl, alkoxy, OH, halo, CN, and CF₃. In some embodiments, heteroaryl^{a} can be a 5-, 6-, 9- or 10-membered mono-or bi-cyclic aromatic ring containing, where possible, 1, 2, 3 or 4 ring members independently selected from N, S, and O; wherein heteroaryl^{a} may be optionally substituted with 1, 2 or 3 substituents independently selected from alkyl, alkoxy, OH, halo, CN, and CF₃. Examples of suitable heteroaryl^{a} groups include thienyl, furanyl, pyrrolyl, pyrazolyl, imidazoyl, oxazolyl, isoxazolyl, thiazolyl, isothiazolyl, triazolyl, oxadiazolyl, thiadiazolyl, tetrazolyl, pyridinyl, pyridazinyl, pyrimidinyl, pyrazinyl, indolyl, benzimidazolyl, benzotriazolyl, quinolinyl, isoquinolinyl, 5-azathianaphthenyl, indolizinyl, isoindolyl, indazolyl, benzothiazolyl, cinnolinyl, quinazolinyl, quinoxalinyl, 1,8-napthyridinyl and phthalazinyl (optionally substituted as stated above). More specifically, “heteroaryla” can be a 9- or 10-membered bi-cyclic ring as defined, and optionally substituted as stated above. Examples of suitable 9- or 10-membered heteroaryl^{a} include indolyl, benzimidazolyl, benzotriazolyl, quinolinyl, isoquinolinyl, 5-azathianaphthenyl, I, indolizinyl, isoindolyl, indazolyl, benzothiazolyl, cinnolinyl, quinazolinyl, quinoxalinyl, 1,8-napthyridinyl and phthalazinyl.
As noted above, “heterocycloalkyl” is a non-aromatic carbon-containing mono- or bi-cyclic ring containing 3, 4, 5, 6, or 7 ring members, wherein at least one ring member is independently selected from N, S, and

O, and wherein, when heterocycloalkyl is a bi-cyclic ring system, the bicyclic ring system is fused, bridged or spiro; heterocycloalkyl may be optionally be substituted with 1 or 2 substituents independently selected from alkyl, cyclopropyl, (C₁-C₆) alkoxy, OH, CN, CF₃, oxo, and halo. More specifically, “heterocycloalkyl” can be a non-aromatic carbon-containing monocyclic ring containing 3, 4, 5, or 6, ring members, wherein at least one ring member is independently selected from N and O; heterocycloalkyl may be optionally substituted with 1 or 2 substituents independently selected from small alkyl, cyclopropyl, small alkoxy, OH, CN, CF₃, oxo, and halo.

“Salt”, as used herein (including “pharmaceutically acceptable salt”) means a physiologically or toxicologically tolerable salt and includes, when appropriate, pharmaceutically acceptable base addition salts and pharmaceutically acceptable acid addition salts. For example (i) where a compound of the invention contains one or more acidic groups, for example carboxy groups, base addition salts (including pharmaceutically acceptable base addition salts) that can be formed include sodium, potassium, calcium, magnesium and ammonium salts, or salts with organic amines, such as, diethylamine, N-methyl-glucamine, diethanolamine or amino acids (e.g. lysine) and the like; (ii) where a compound of the invention contains a basic group, such as an amino group, acid addition salts (including pharmaceutically acceptable acid addition salts) that can be formed include hydrochlorides, hydrobromides, sulfates, phosphates, acetates, citrates, lactates, tartrates, mesylates, succinates, oxalates, phosphates, esylates, tosylates, benzenesulfonates, naphthalenedisulphonates, maleates, adipates, fumarates, hippurates, camphorates, xinafoates, p-acetamidobenzoates, dihydroxybenzoates, hydroxynaphthoates, succinates, ascorbates, oleates, bisulfates, trifluoroacetates and the like.

Hemisalts of acids and bases can also be formed, for example, hemisulfate and hemicalcium salts.

For a review of suitable salts, see “Handbook of Pharmaceutical Salts: Properties, Selection and Use” by Stahl and Wermuth (Wiley-VCH, Weinheim, Germany, 2002).

“Prodrug” refers to a compound which is convertible in vivo by metabolic means (e.g. by hydrolysis, reduction or oxidation) to a compound of the invention. Suitable groups for forming prodrugs are described in ‘The Practice of Medicinal Chemistry, 2^{nd} Ed. pp561-585 (2003) and in F. J. Leinweber, Drug Metab. Res., 1987, 18, 379.

The compounds of the invention can exist in both unsolvated and solvated forms. The term ‘solvate’ is used herein to describe a molecular complex comprising the compound of the invention and a stoichiometric amount of one or more pharmaceutically acceptable solvent molecules, for example, ethanol. The term ‘hydrate’ is employed when the solvent is water.

Where compounds of the invention exist in one or more geometric, optical, enantiomeric, diastereomeric and tautomeric forms, including but not limited to cis- and trans-forms, E- and Z-forms, R-, S- and meso-forms, keto-, and enol-forms. Unless otherwise stated a reference to a particular compound includes all such isomeric forms, including racemic and other mixtures thereof. Where appropriate such isomers can be separated from their mixtures by the application or adaptation of known methods (e.g. chromatographic techniques and recrystallisation techniques). Where appropriate such isomers can be prepared by the application or adaptation of known methods (e.g. asymmetric synthesis). For example, where compounds of the invention exist as a mixture of stereoisomers, one stereoisomer can be present at a purity of >90% relative to the remaining stereoisomers, or more specifically at a purity of >95% relative to the remaining stereoisomers, or yet more specifically at a purity of >99% relative to the remaining stereoisomers. For example, where compounds of the invention exists in enantiomeric forms, the compound can be >90% enantiomeric excess (ee), or more specifically >95% enantiomeric excess (ee), or yet more specifically, >99% ee.

Unless otherwise stated, the compounds of the invention include compounds that differ only in the presence of one or more isotopically enriched atoms. For example, compounds wherein hydrogen is replaced by deuterium or tritium, or wherein carbon is replaced by 13C or 14C, are within the scope of the present invention. Such compounds are useful, for example, as analytical tools or probes in biological assays.

Methods for the preparation of deuterated analogues will be readily apparent to those skilled in the art. For example, methods may include the use of deuterated starting materials in the synthesis of the compounds described herein. Deuterated starting materials will be readily available to the skilled person, from standard commercial sources. Methods for making deuterated analogues and deuterated starting materials may also include deuterium exchange. For example, deuterium exchange may be achieved by mixing the compounds with D2O.

In the context of the present invention, references herein to “treatment” include references to curative, palliative and prophylactic treatment. For instance, treatment includes preventing the symptoms of the disease conditions for which FXIIa is a causative factor.

### Methods

The compounds of the invention may be administered alone or in combination with one or more other compounds of the invention or in combination with one or more other drugs (or as any combination thereof). Generally, they will be administered as a formulation in association with one or more pharmaceutically acceptable excipients. The term ‘excipient’ is used herein to describe any ingredient other than the compound(s) of the invention which may impart either a functional (i.e., drug release rate controlling) and/or a non-functional (i.e., processing aid or diluent) characteristic to the formulations. The choice of excipient will to a large extent depend on factors such as the particular mode of administration, the effect of the excipient on solubility and stability, and the nature of the dosage form.

Compounds of the invention intended for pharmaceutical use may be administered as a solid or liquid, such as a tablet, capsule or solution. Pharmaceutical compositions suitable for the delivery of compounds of the present invention and methods for their preparation will be readily apparent to those skilled in the art. Such compositions and methods for their preparation may be found, for example, in Remington's Pharmaceutical Sciences, 19th Edition (Mack Publishing Company, 1995).
Accordingly, the present invention provides a pharmaceutical composition comprising a compound of the invention and a pharmaceutically acceptable carrier, diluent or excipient.

For the treatment of conditions such as retinal vascular permeability associated with diabetic retinopathy and diabetic macular edema, the compounds of the invention may be administered in a form suitable for injection into the ocular region of a patient, in particular, in a form suitable for intra-vitreal injection. It is envisaged that formulations suitable for such use will take the form of sterile solutions of a compound of the invention in a suitable aqueous vehicle. The compositions may be administered to the patient under the supervision of the attending physician.

The compounds of the invention may also be administered directly into the blood stream, into subcutaneous tissue, into muscle, or into an internal organ. Suitable means for parenteral administration include intravenous, intraarterial, intraperitoneal, intrathecal, intraventricular, intraurethral, intrasternal, intracranial, intramuscular, intrasynovial and subcutaneous. Suitable devices for parenteral administration include needle (including microneedle) injectors, needle-free injectors and infusion techniques.

Parenteral formulations are typically aqueous or oily solutions. Where the solution is aqueous, they may contain excipients such as sugars (including but not restricted to glucose, manitol, sorbitol, etc.), salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water.

Parenteral formulations may include implants derived from degradable polymers such as polyesters (i.e., polylactic acid, polylactide, polylactide-co-glycolide, polycapro-lactone, polyhydroxybutyrate), polyorthoesters and polyanhydrides. These formulations may be administered via surgical incision into the subcutaneous tissue, muscular tissue or directly into specific organs.

The preparation of parenteral formulations under sterile conditions, for example, by lyophilisation, may readily be accomplished using standard pharmaceutical techniques well known to those skilled in the art.

The solubility of compounds of the invention used in the preparation of parenteral solutions may be increased by the use of appropriate formulation techniques, such as the incorporation of co-solvents and/or solubility-enhancing agents such as surfactants, micelle structures and cyclodextrins.

The compounds of the invention may be administered orally. Oral administration may involve swallowing, so that the compound enters the gastrointestinal tract, and/or buccal, lingual, or sublingual administration by which the compound enters the blood stream directly from the mouth.

Formulations suitable for oral administration include solid plugs, solid microparticulates, semi-solids and liquids (including multiple phases or dispersed systems). Exemplary formulations suitable for oral administration include tablets; soft or hard capsules containing multi-or nano-particulates, liquids, emulsions or powders; lozenges (including liquid-filled); chews; gels; fast dispersing dosage forms; films; ovules; sprays; and buccal/mucoadhesive patches.
Liquid (including multiple phases and dispersed systems) formulations include emulsions, solutions, syrups and elixirs. Such formulations may be presented as fillers in soft or hard capsules (made, for example, from gelatin or hydroxypropylmethylcellulose) and typically comprise a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

The compounds of the invention may also be used in fast-dissolving, fast-disintegrating dosage forms such as those described in Liang and Chen, Expert Opinion in Therapeutic Patents, 2001, 11 (6), 981-986.

The formulation of tablets is discussed in Pharmaceutical Dosage Forms: Tablets, Vol. 1, by H. Lieberman and L. Lachman (Marcel Dekker, New York, 1980).

For administration to human patients, the total daily dose of the compounds of the invention is typically in the range 0.1 mg and 10,000 mg, or between 1 mg and 5000 mg, or between 10 mg and 1000 mg depending, of course, on the mode of administration.

The total dose may be administered in single or divided doses and may, at the physician's discretion, fall outside of the typical range given herein. These dosages are based on an average human subject having a weight of about 60 kg to 70 kg. The physician will readily be able to determine doses for subjects whose weight falls outside this range, such as infants and the elderly.

### Numbered embodiments

1. A compound of Formula (I),

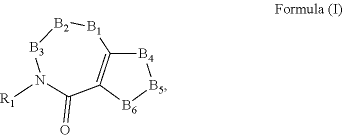

wherein:
B₁ is a carbon centre or nitrogen centre; and either:
(i) B₂ and B₃ together form a single bond between B₁ and the nitrogen atom to which R₁ is attached,
(ii) B₂ is a carbon centre or a nitrogen centre, and B₃ is a single bond between B₂ and the nitrogen atom to which R₁ is attached,
(iii) B₂ is a carbon centre and B₃ is a nitrogen centre, or
(iv) B₂ and B₃ are both carbon centres,
wherein, when B₂ is a carbon centre or nitrogen centre, B₁ is connected to B₂ via either a single or double bond;
wherein, when both B₂ and B₃ are present, B₂ and B₃ are connected via either a single or double bond;
wherein B₁ may, where possible, be optionally substituted with 1 or 2 substituents selected from alkyl, cycloalkyl, alkoxy, OH, CF₃, CN, and halo, or 1 oxo substituent, or 2 substituents which, together with B₁ to which they are both attached, form a 3-, 4-, or 5-membered non-aromatic monocyclic carbon-containing ring, optionally wherein one or two ring members are selected from N, S, and O;
wherein, when B₂ is a carbon centre, B₂ may be optionally substituted with 1 or 2 substituents selected from alkyl, cycloalkyl, alkoxy, OH, CF₃, CN, and halo, or 1 oxo substituent; and
wherein, when B₃ is a carbon centre, B₃ may be optionally substituted with 1 or 2 substituents selected from alkyl, cycloalkyl, alkoxy, OH, CF₃, CN, and halo, or 1 oxo substituent;
and either:
(i) B₄ is N(R₂), B₅ is N, and B₆ is C(R₃), and B₄ is connected to B₅ via a single bond and B₅ is connected to B₆ via a double bond;
(ii) B₄ is C(R₂), B₅ is N, and B₆ is N(R₃), and B₄ is connected to B₅ via a double bond and B₅ is connected to B₆ via a single bond; or
(iii) B₄ is C(R₂), B₅ is C(H), and B₆ is N(R₃), and B₄ is connected to B₅ via a double bond and B₅ is connected to B₆ via a single bond;
R₂ is selected from H, —(CH₂)₀₋₃—C(═O)—N(R₄)(R₅), —C(═O)—O—R₆, —(CH₂)₀₋₃—NR₁₄—C(═O)R₁₅, —(CH₂)₀₋₃—NR₃₄—S(═O)₂R₃₅, alkyl, cycloalkyl, heterocycloalkyl, alkoxy, —OH, —CF₃, halo, aryl, heteroaryl, —CH₂—O—R₁₆ and —CN;
wherein R₄ is H, alkyl, cycloalkyl, and heterocycloalkyl, and
R₅ is selected from alkyl, —OH, —S(═O)₂-(small alkyl), —(CH₂)₁₋₃—(C═O)—OR₃₃, —(CH₂)₁₋₃—(C═O)—N(R₃₆)(R₃₇), —(CH₂)₀₋₃-cycloalkyl^{b}, —(CH₂)₀₋₃-heterocycloalkyl, (CH(alkyl))-heterocycloalkyl, —(CH₂)₀₋₃-polycycloalkyl, —(CH₂)₀₋₃-aryl, —(C(H)(phenyl))-alkyl, (CH₂)₀₋₃heteroaryl^{a}, —(C(H)(heteroaryl))-alkyl, —(C(H)(alkyl))-(C═O)—OR₃₈, —(C(H)(alkyl))-(C═O)—N(R₃₉)(R₄₀), —(C(H)(heteroaryl))-(C═O)—OR₂₈, and (C(H)(heteroaryl))-(C═O)—N(R₄₁)(R₄₂);
or wherein R₄ and R₅ together with the nitrogen atom to which they are attached form a carbon-containing 4-, 5-, 6-, or 7-membered heterocyclic ring which may be saturated or unsaturated with 1 or 2 double bonds, optionally containing one or two additional heteroatoms selected from N, O, and S, wherein the ring may be optionally substituted with 1, 2, or 3 substituents selected from oxo, alkyl, alkoxy, OH, halo and CF_{3;}
R₂₈, R₃₃ R₃₆, R₃₇, R₃₈, R₃₉, R₄₀, R₄₁, and R₄₂ are each independently selected from H and small alkyl;
R₆ is selected from H, alkyl, —(CH₂)₀₋₃-cycloalkyl, —(CH₂)₀₋₃-heterocycloalkyl, —(CH(alkyl))-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, and —(C(H)(heteroaryl))-alkyl;
R₁₄ is selected from H, alkyl, cycloalkyl, and heterocycloalkyl;
R₁₅ is selected from H, alkyl, —(CH₂)₀₋₃-cycloalkyl, —(CH₂)₀₋₃-heterocycloalkyl, (CH(alkyl))-heterocycloalkyl, —(CH₂)₀₋₃-aryl, —(C(H)(phenyl))-alkyl, —(CH₂)₀₋₃heteroaryl^{a}, and —(C(H)(heteroaryl))-alkyl;
R₁₆ is selected from H, alkyl, cycloalkyl, —(CH₂)₁₋₃—(C═O)—OH, heterocycloalkyl, and —(CH₂)₀₋₃heteroaryl;
R₃₄ is selected from H, alkyl, cycloalkyl, and heterocycloalkyl;
R₃₅ is selected from H, alkyl, —(CH₂)₀₋₃-cycloalkyl, —(CH₂)₀₋₃-heterocycloalkyl, —(CH(alkyl)-heterocycloalkyl, —(CH₂)₀₋₃-aryl, —(C(H)(phenyl))-alkyl, —(CH₂)₀₋₃heteroaryl^{a}, and —(C(H)(heteroaryl))-alkyl;
R₁ is:
Formula (II),

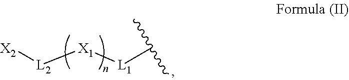

wherein:
n is 0 or 1;
L₁ is a linker selected from a single bond, —(CH₂)₁₋₃—, —(CH)(alkyl)-, and —C(alkyl)(alkyl); X₁ is phenyl or a 5- or 6-membered carbon-containing aromatic ring containing one, two or three ring members that are selected from N, S, and O;
wherein X₁ may be optionally substituted with 1 or 2 substituents independently selected from alkyl, cycloalkyl, alkoxy, OH, O(cyclopropyl), CF₃, and halo;
L₂ is a linker selected from a single bond, —(CH₂)₁₋₃—, —(CH)(alkyl)-, —C(alkyl)(alkyl)-, —C(═O)—, and —SO₂—; and
X₂ is either:
(a) —NR₂₀R₂₁,
wherein R₂₀ is selected from H, alkyl, and —(CH₂)₀₋₃—R_{22;}
wherein R₂₁ is selected from H, alkyl, and —(CH₂)₀₋₃—R₂₃;
wherein R₂₂ is selected from cycloalkyl, heterocycloalkyl, aryl, heteroaryl,

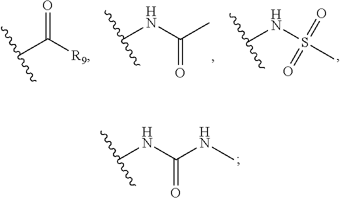

wherein R₂₃ is selected from cycloalkyl, heterocycloalkyl, aryl, heteroaryl,

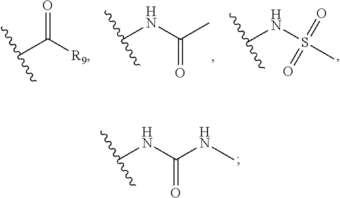

or
(b) 2-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and
   R₁₉, wherein R₁₈ and R₁₉ are independently selected from alkyl, cycloalkyl, alkoxy, OH, oxo, CF₃, and halo,
   or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O, and X₂ may be further optionally substituted with 1 or 2 substituents R₂₉ and R₃₀, wherein R₂₉ and R₃₀ are independently selected from alkyl, cycloalkyl, alkoxy, OH, oxo, CF₃, and halo; or,
(ii) Formula (III) comprising an aromatic ring A fused to a non-aromatic ring A′,

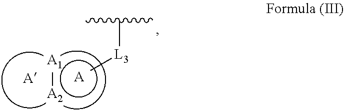

wherein:
L₃ is a linker selected from a single bond, —(CH₂)₁₋₃—, —(CH)(alkyl)-, and —C(alkyl)(alkyl);
A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, O(cyclopropyl), CF₃, and halo;
A′ is a non-aromatic carbon-containing monocyclic ring containing 5, 6, or 7, ring members, wherein at least one ring member is independently selected from N, S, and O; wherein A′ may be optionally substituted with one R₇, wherein R₇ is selected from alkyl, alkoxy, and —(CH₂)₀₋₃—R₈,
wherein R₈ is selected from cycloalkyl, heterocycloalkyl, aryl, heteroaryl,

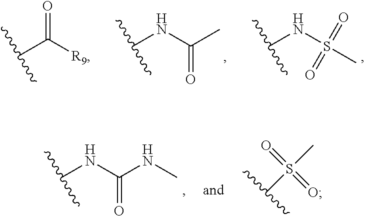

wherein R₉ is selected from H, alkyl, OH, N (R₃₁)(R₃₂), and alkoxy;
wherein R₃₁ is H or small alkyl;
wherein R₃₂ is selected from H, small alkyl, and —(CH₂)₀₋₃heteroaryl;
wherein A′ may be optionally substituted with 1, 2, or 3 further substituents R₁₀, R₁₁, and
R₁₇, wherein R₁₀, R₁₁, and R₁₇, are independently selected from alkyl, cycloalkyl, alkoxy, OH, oxo, CF₃, and halo,
or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O,
or wherein A′ may be optionally substituted with 1 or 2 further substituents R₄₃ and R₄₄ independently selected from alkyl, cycloalkyl, alkoxy, OH, oxo, CF₃, and halo, and two different carbon ring members within A′ are connected by a bridging —CH₂-group; and
R₃ is phenyl, or a 5- or 6-membered carbon-containing aromatic ring containing one, two or three ring members that are selected from N, S, and O, wherein R₃ is substituted with one, two, or three substituents independently selected from small alkyl^{b}, small alkoxy, cyclopropyl, OH, NH₂, CF₃, halo, —C(═O)—NH₂, —C(═O)—N(H)(small alkyl), —C(═O)—N(small alkyl)(small alkyl), and CN; and
alkyl is a linear saturated hydrocarbon having up to 10 carbon atoms (C₁-C₁₀) or a branched saturated hydrocarbon of between 3 and 10 carbon atoms (C₃-C₁₀); alkyl may optionally be substituted with 1, 2 or 3 substituents independently selected from cyclopropyl, (C₁-C₆) alkoxy, OH, —NR₁₂R₁₃, —C(═O) NR₁₂R₁₃, CN, CF₃, and halo;
small alkyl is a linear saturated hydrocarbon having up to 4 carbon atoms (C₁-C₄) or a branched saturated hydrocarbon of between 3 and 4 carbon atoms (C₃-C₄); small alkyl may optionally be substituted with 1 or 2 substituents independently selected from cyclopropyl, OH, CN, CF₃, NH₂, —C(═O)NH₂, and halo;
small alkyl^{b} is a linear saturated hydrocarbon having up to 4 carbon atoms (C₁-C₄) or a branched saturated hydrocarbon of between 3 and 4 carbon atoms (C₃-C₄); small alkyl may optionally be substituted with 1 or 2 substituents independently selected from cyclopropyl, OH, OMe, OEt, OⁱPr, OⁿPr, O(cyclopropyl), OCF₃, OCHF₂, CN, CF₃, NH₂, —C(═O)NH₂, and halo;
alkoxy is OCF₃, O(cyclopropyl), a linear O-linked hydrocarbon of between 1 and 6 carbon atoms (C₁-C₆), or a branched O-linked hydrocarbon of between 3 and 6 carbon atoms (C₃-C₆); alkoxy may optionally be substituted with 1 or 2 substituents independently selected from cyclopropyl, OH, CN, CF₃, and halo;
small alkoxy is OCF₃, O(cyclopropyl), a linear O-linked hydrocarbon of between 1 and 4 carbon atoms (C₁-C₄), or a branched O-linked hydrocarbon of between 3 and 4 carbon atoms (C₃-C₄); small alkoxy may optionally be substituted with 1 or 2 substituents independently selected from cyclopropyl, OH, CN, CF₃, and halo;
aryl is phenyl, biphenyl or naphthyl; aryl may be optionally substituted with 1, 2 or 3 substituents independently selected from alkyl, alkoxy, methylenedioxy, ethylenedioxy, OH, halo, CN, (CH₂)₀₋₃—NR₁₂R₁₃, OCF₃ and CF_{3;}
polycycloalkyl is a bicyclic saturated hydrocarbon ring system of between 5 and 8 carbon atoms (C₅-C₈), wherein the bicyclic ring is bridged, or is adamantyl; polycycloalkyl may optionally be substituted with 1 or 2 substituents independently selected from alkyl, (C₁-C₆) alkoxy, OH, CN, CF₃, and halo;
cycloalkyl is a monocyclic saturated hydrocarbon ring of between 3 and 6 carbon atoms (C₃-C₆); cycloalkyl may optionally be substituted with 1 or 2 substituents independently selected from alkyl, (C₁-C₆) alkoxy, OH, CN, CF₃, and halo;
cycloalkyl^{a} is a monocyclic saturated hydrocarbon ring of between 3 and 6 carbon atoms (C₃-C₆); cycloalkyl may optionally be substituted with 1 or 2 substituents independently selected from alkyl, (C₁-C₆) alkoxy, OH, CN, CF₃, halo, and —(C═O)—OH;
cycloalkyl^{b} is a monocyclic saturated hydrocarbon ring of between 3 and 6 carbon atoms (C₃-C₆); cycloalkyl may optionally be substituted with 1 or 2 substituents independently selected from alkyl, (C₁-C₆) alkoxy, OH, NH₂, NH(small alkyl), CN, CF₃, halo, and —(C═O)—OH;
halo is F, Cl, Br, or I;
heteroaryl is a 5- or 6-membered carbon-containing aromatic ring containing one, two or three ring members that are selected from N, S, and O; wherein heteroaryl may be optionally substituted with 1, 2 or 3 substituents independently selected from alkyl, alkoxy, cyclopropyl, OH, halo, CN, and CF_{3;}
heteroaryl^{a} is a 5-, 6-, 9- or 10-membered mono- or bi-cyclic aromatic ring containing, where possible, 1, 2, 3 or 4 ring members independently selected from N, S, and O; wherein heteroaryl^{a} may be optionally substituted with 1, 2 or 3 substituents independently selected from alkyl, alkoxy, cyclopropyl, OH, halo, CN, and CF_{3;}
heterocycloalkyl is a non-aromatic carbon-containing mono- or bi-cyclic ring containing 3, 4, 5, 6, or 7 ring members, wherein at least one ring member is independently selected from N, S, and O; heterocycloalkyl may optionally be substituted with 1 or 2 substituents independently selected from alkyl, cyclopropyl, (C₁-C₅) alkoxy, OH, CN, CF₃, oxo, and halo; wherein when heterocycloalkyl is a bi-cyclic ring system, the bicyclic ring system is fused, bridged or spiro;
each R₁₂ and R₁₃ is independently selected from H, alkyl, cycloalkyl, and heterocycloalkyl;
and tautomers, isomers, stereoisomers (including enantiomers, diastereoisomers and racemic and scalemic mixtures thereof), deuterated analogues, and pharmaceutically acceptable salts and/or solvates thereof.

2. A compound of Formula (I),

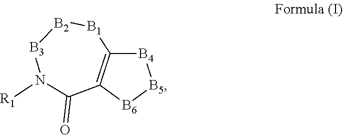

wherein:
B₁ is a carbon centre or nitrogen centre; and either:
(i) B₂ and B₃ together form a single bond between B₁ and the nitrogen atom to which R₁ is attached,
(ii) B₂ is a carbon centre or a nitrogen centre, and B₃ is a single bond between B₂ and the nitrogen atom to which R₁ is attached,
(iii) B₂ is a carbon centre and B₃ is a nitrogen centre, or
(iv) B₂ and B₃ are both carbon centres,
wherein, when B₂ is a carbon centre or nitrogen centre, B₁ is connected to B₂ via either a single or double bond;
wherein, when both B₂ and B₃ are present, B₂ and B₃ are connected via either a single or double bond;
wherein B₁ may, where possible, be optionally substituted with 1 or 2 substituents selected from alkyl, cycloalkyl, alkoxy, OH, CF₃, CN, and halo, or 1 oxo substituent, or 2 substituents which, together with B₁ to which they are both attached, form a 3-, 4-, or 5-membered non-aromatic monocyclic carbon-containing ring, optionally wherein one or two ring members are selected from N, S, and O;
wherein, when B₂ is a carbon centre, B₂ may be optionally substituted with 1 or 2 substituents selected from alkyl, cycloalkyl, alkoxy, OH, CF₃, CN, and halo, or 1 oxo substituent; and
wherein, when B₃ is a carbon centre, B₃ may be optionally substituted with 1 or 2 substituents selected from alkyl, cycloalkyl, alkoxy, OH, CF₃, CN, and halo, or 1 oxo substituent;
and either:
(i) B₄ is N(R₂), B₅ is N, and B₆ is C(R₃), and B₄ is connected to B₅ via a single bond and B₅ is connected to B₆ via a double bond;
(ii) B₄ is C(R₂), B₅ is N, and B₆ is N(R₃), and B₄ is connected to B₅ via a double bond and B₅ is connected to B₆ via a single bond; or
(iii) B₄ is C(R₂), B₅ is C(H), and B₆ is N(R₃), and B₄ is connected to B₅ via a double bond and B₅ is connected to B₆ via a single bond;
R₂ is selected from H, —(CH₂)₀₋₃—C(═O)—N(R₄)(R₅), —C(═O)—O—R₆, —(CH₂)₀₋₃—NR₁₄—C(═O)R₁₅, —(CH₂)₀₋₃—NR₃₄—S(═O)₂R₃₅, alkyl, cycloalkyl, heterocycloalkyl, alkoxy, —OH, —CF₃, halo, aryl, heteroaryl, —CH₂—O—R₁₆ and —CN;
wherein R₄ is H, alkyl, cycloalkyl, and heterocycloalkyl, and
R₅ is selected from alkyl, —OH, —S(═O)₂-(small alkyl), —(CH₂)₁₋₃—(C═O)—OR₃₃, —(CH₂)₁₋₃—(C═O)—N(R₃₆)(R₃₇), —(CH₂)₀₋₃-cycloalkyl^{a}, —(CH₂)₀₋₃-heterocycloalkyl, —(CH(alkyl))-heterocycloalkyl, —(CH₂)₀₋₃-polycycloalkyl, —(CH₂)₀₋₃-aryl, —(C(H)(phenyl))-alkyl, —(CH₂)₀₋₃heteroaryl^{a}, —(C(H)(heteroaryl))-alkyl, —(C(H)(alkyl))-(C═O)—OR₃₈, —(C(H)(alkyl))-(C═O)—N(R₃₉)(R₄₀), —(C(H)(heteroaryl))-(C═O)—OR₂₈, and —(C(H)(heteroaryl))-(C═O)—N(R₄₁)(R₄₂);
or wherein R₄ and R₅ together with the nitrogen atom to which they are attached form a carbon-containing 4-, 5-, 6-, or 7-membered heterocyclic ring which may be saturated or unsaturated with 1 or 2 double bonds, optionally containing one or two additional heteroatoms selected from N, O, and S, wherein the ring may be optionally substituted with 1, 2, or 3 substituents selected from oxo, alkyl, alkoxy, OH, halo and CF_{3;}
R₂₈, R₃₃ R₃₆, R₃₇, R₃₈, R₃₉, R₄₀, R₄₁, and R₄₂ are each independently selected from H and small alkyl;
R₆ is selected from H, alkyl, —(CH₂)₀₋₃-cycloalkyl, —(CH₂)₀₋₃-heterocycloalkyl, —(CH(alkyl))-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, and —(C(H)(heteroaryl))-alkyl;
R₁₄ is selected from H, alkyl, cycloalkyl, and heterocycloalkyl;
R₁₅ is selected from H, alkyl, —(CH₂)₀₋₃-cycloalkyl, —(CH₂)₀₋₃-heterocycloalkyl, —(CH(alkyl))-heterocycloalkyl, —(CH₂)₀₋₃-aryl, —(C(H)(phenyl))-alkyl, —(CH₂)₀₋₃heteroaryl^{a}, and —(C(H)(heteroaryl))-alkyl;
R₁₆ is selected from H, alkyl, cycloalkyl, —(CH₂)₁₋₃—(C═O)—OH, heterocycloalkyl, and —(CH₂)₀₋₃heteroaryl;
R₃₄ is selected from H, alkyl, cycloalkyl, and heterocycloalkyl;
R₃₅ is selected from H, alkyl, —(CH₂) 03-cycloalkyl, —(CH₂)₀₋₃-heterocycloalkyl, —(CH(alkyl)-heterocycloalkyl, —(CH₂)₀₋₃-aryl, —(C(H)(phenyl))-alkyl, —(CH₂)₀₋₃heteroaryl^{a}, and —(C(H)(heteroaryl))-alkyl;
R₁ is:

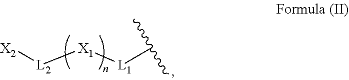

wherein:
n is 0 or 1;
L₁ is a linker selected from a single bond, —(CH₂)₁₋₃—, —(CH)(alkyl)-, and —C(alkyl)(alkyl)-;
X₁ is phenyl or a 5- or 6-membered carbon-containing aromatic ring containing one, two or three ring members that are selected from N, S, and O;
wherein X₁ may be optionally substituted with 1 or 2 substituents independently selected from alkyl, cycloalkyl, alkoxy, OH, CF₃, and halo;
L₂ is a linker selected from a single bond, —(CH₂)₁₋₃—, —(CH)(alkyl)-, —C(alkyl)(alkyl)-, —C(═O)—, and —SO₂—; and
X₂ is either:
wherein R₂₀ is selected from H, alkyl, and —(CH₂)₀₋₃—R_{22;}
wherein R₂₁ is selected from H, alkyl, and —(CH₂)₀₋₃—R_{23;}
(a) —NR₂₀R₂₁,

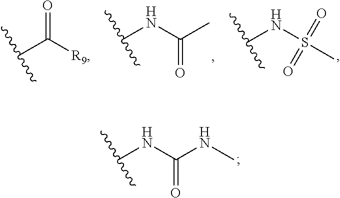

wherein R₂₂ is selected from cycloalkyl, heterocycloalkyl, aryl, heteroaryl,

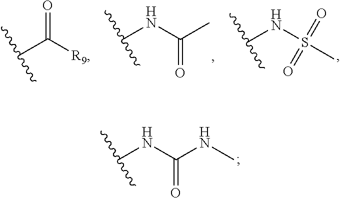

wherein R₂₃ is selected from cycloalkyl, heterocycloalkyl, aryl, heteroaryl, or
(b) 2-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O, and X₂ may be further optionally substituted with 1 or 2 substituents R₂₉ and R₃₀, wherein R₂₉ and R₃₀ are independently selected from alkyl, cycloalkyl, alkoxy, OH, oxo, CF₃, and halo; or,
(ii) Formula (III) comprising on aromatic ring A fused to a non-aromatic ring A′,

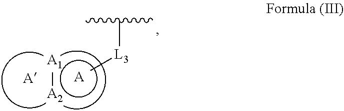

wherein:
L₃ is a linker selected from a single bond, —(CH₂)₁₋₃—, —(CH)(alkyl)-, and —C(alkyl)(alkyl)-;
A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, CF₃, and halo;
A′ is a non-aromatic carbon-containing monocyclic ring containing 5, 6, or 7, ring members, wherein at least one ring member is independently selected from N, S, and O; wherein A′ may be optionally substituted with one R₇, wherein R₇ is selected from alkyl, alkoxy, and —(CH₂)₀₋₃—R₈,

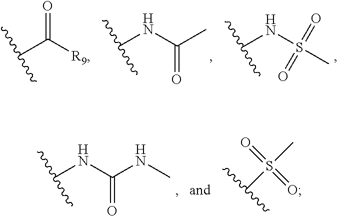

wherein R₈ is selected from cycloalkyl, heterocycloalkyl, aryl, heteroaryl, wherein R₉ is selected from H, alkyl, OH, N (R₃₁)(R₃₂), and alkoxy;
wherein R₃₁ is H or small alkyl;
wherein R₃₂ is selected from H, small alkyl, and —(CH₂)₀₋₃heteroaryl;
wherein A′ may be optionally substituted with 1, 2, or 3 further substituents R₁₀, R₁₁, and
R₁₇, wherein R₁₀, R₁₁, and R₁₇, are independently selected from alkyl, cycloalkyl, alkoxy, OH, Oxo, CF₃, and halo, or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O; and
R₃ is phenyl, or a 5- or 6-membered carbon-containing aromatic ring containing one, two or three ring members that are selected from N, S, and O, wherein R₃ is substituted with one or two substituents independently selected from small alkyl, small alkoxy, cyclopropyl, OH, CF₃, halo, —C(═O)—NH₂, —C(═O)—N(H)(small alkyl), —C(═O)—N(small alkyl)(small alkyl), and CN; and
alkyl is a linear saturated hydrocarbon having up to 10 carbon atoms (C₁-C₁₀) or a branched saturated hydrocarbon of between 3 and 10 carbon atoms (C₃-C₁₀); alkyl may optionally be substituted with 1, 2 or 3 substituents independently selected from cyclopropyl, (C₁-C₆) alkoxy, OH, —NR₁₂R₁₃, —C(═O) NR₁₂R₁₃, CN, CF₃, and halo;
small alkyl is a linear saturated hydrocarbon having up to 4 carbon atoms (C₁-C₄) or a branched saturated hydrocarbon of between 3 and 4 carbon atoms (C₃-C₄); small alkyl may optionally be substituted with 1 or 2 substituents independently selected from cyclopropyl, OH, CN, CF₃, NH₂, —C(═O)NH₂, and halo;
alkoxy is OCF₃, a linear O-linked hydrocarbon of between 1 and 6 carbon atoms (C₁-C₆), or a branched O-linked hydrocarbon of between 3 and 6 carbon atoms (C₃-C₆); alkoxy may optionally be substituted with 1 or 2 substituents independently selected from cyclopropyl, OH, CN, CF₃, and halo;
small alkoxy is OCF₃, a linear O-linked hydrocarbon of between 1 and 4 carbon atoms (C₁-C₄), or a branched O-linked hydrocarbon of between 3 and 4 carbon atoms (C₃-C₄); small alkoxy may optionally be substituted with 1 or 2 substituents independently selected from cyclopropyl, OH, CN, CF₃, and halo;
aryl is phenyl, biphenyl or naphthyl; aryl may be optionally substituted with 1, 2 or 3 substituents independently selected from alkyl, alkoxy, methylenedioxy, ethylenedioxy, OH, halo, CN, —(CH₂)₀₋₃—NR₁₂R₁₃, OCF₃ and CF_{3;}
polycycloalkyl is a bicyclic saturated hydrocarbon ring system of between 5 and 8 carbon atoms (C₅-C₈), wherein the bicyclic ring is bridged, or is adamantyl; polycycloalkyl may optionally be substituted with 1 or 2 substituents independently selected from alkyl, (C₁-C₅) alkoxy, OH, CN, CF₃, and halo;
cycloalkyl is a monocyclic saturated hydrocarbon ring of between 3 and 6 carbon atoms (C₃-C₆);
cycloalkyl may optionally be substituted with 1 or 2 substituents independently selected from alkyl, (C₁-C₆) alkoxy, OH, CN, CF₃, and halo;
cycloalkyl^{a} is a monocyclic saturated hydrocarbon ring of between 3 and 6 carbon atoms (C₃-C₆);
cycloalkyl may optionally be substituted with 1 or 2 substituents independently selected from alkyl, (C₁-C₆) alkoxy, OH, CN, CF₃, halo, and —(C═O)—OH;
halo is F, Cl, Br, or I;
heteroaryl is a 5- or 6-membered carbon-containing aromatic ring containing one, two or three ring members that are selected from N, S, and O; wherein heteroaryl may be optionally substituted with 1, 2 or 3 substituents independently selected from alkyl, alkoxy, cyclopropyl, OH, halo, CN, and CF_{3;}
heteroaryl^{a} is a 5-, 6-, 9- or 10-membered mono- or bi-cyclic aromatic ring containing, where possible, 1, 2, 3 or 4 ring members independently selected from N, S, and O; wherein heteroaryl^{a} may be optionally substituted with 1, 2 or 3 substituents independently selected from alkyl, alkoxy, cyclopropyl, OH, halo, CN, and CF_{3;}
heterocycloalkyl is a non-aromatic carbon-containing mono- or bi-cyclic ring containing 3, 4, 5, 6, or 7 ring members, wherein at least one ring member is independently selected from N, S, and O; heterocycloalkyl may optionally be substituted with 1 or 2 substituents independently selected from alkyl, cyclopropyl, (C₁-C₆) alkoxy, OH, CN, CF₃, oxo, and halo; wherein when heterocycloalkyl is a bi-cyclic ring system, the bicyclic ring system is fused, bridged or spiro;
each R₁₂ and R₁₃ is independently selected from H, alkyl, cycloalkyl, and heterocycloalkyl;

and tautomers, isomers, stereoisomers (including enantiomers, diastereoisomers and racemic and scalemic mixtures thereof), deuterated analogues, and pharmaceutically acceptable salts and/or solvates thereof.

3. A compound of Formula (I),

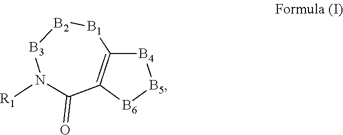

wherein:
B₁ is a carbon centre or nitrogen centre; and either:
(i) B₂ and B₃ together form a single bond between B₁ and the nitrogen atom to which R₁ is attached,
(ii) B₂ is a carbon centre or a nitrogen centre, and B₃ is a single bond between B₂ and the nitrogen atom to which R₁ is attached,
(iii) B₂ is a carbon centre and B₃ is a nitrogen centre, or
(iv) B₂ and B₃ are both carbon centres,
wherein, when B₂ is a carbon centre or nitrogen centre, B₁ is connected to B₂ via either a single or double bond;
wherein, when both B₂ and B₃ are present, B₂ and B₃ are connected via either a single or double bond;
wherein B₁ may, where possible, be optionally substituted with 1 or 2 substituents selected from alkyl, cycloalkyl, alkoxy, OH, CF₃, CN, and halo, or 1 oxo substituent, or 2 substituents which, together with B₁ to which they are both attached, form a 3-, 4-, or 5-membered non-aromatic monocyclic carbon-containing ring, optionally wherein one or two ring members are selected from N, S, and O;
wherein, when B₂ is a carbon centre, B₂ may be optionally substituted with 1 or 2 substituents selected from alkyl, cycloalkyl, alkoxy, OH, CF₃, CN, and halo, or 1 oxo substituent; and
wherein, when B₃ is a carbon centre, B₃ may be optionally substituted with 1 or 2 substituents selected from alkyl, cycloalkyl, alkoxy, OH, CF₃, CN, and halo, or 1 oxo substituent;
and either:
(i) B₄ is N(R₂), B₅ is N, and B₆ is C(R₃), and B₄ is connected to B₅ via a single bond and B₅ is connected to B₆ via a double bond;
(ii) B₄ is C(R₂), B₅ is N, and B₆ is N(R₃), and B₄ is connected to B₅ via a double bond and B₅ is connected to B₆ via a single bond; or
(iii) B₄ is C(R₂), B₅ is C(H), and B₆ is N(R₃), and B₄ is connected to B₅ via a double bond and B₅ is connected to B₆ via a single bond;
R₂ is selected from H, —(CH₂)₀₋₃—C(═O)—N(R₄)(R₅), —C(═O)—O—R₆, —(CH₂)₀₋₃—NR₁₄—C(═O)R₁₅, —(CH₂)₀₋₃—NR₃₄—S(═O)₂R₃₅, alkyl, cycloalkyl, heterocycloalkyl, alkoxy, —OH, —CF₃, halo, aryl, heteroaryl, —CH₂—O—R₁₆ and —CN;
wherein R₄ is H, alkyl, cycloalkyl, and heterocycloalkyl, and
R₅ is selected from alkyl, —(CH₂)₀₋₃-cycloalkyl, —(CH₂)₀₋₃-heterocycloalkyl, —(CH(alkyl))-heterocycloalkyl, —(CH₂)₀₋₃-polycycloalkyl, —(CH₂)₀₋₃-aryl, —(C(H)(phenyl))-alkyl, —(CH₂)₀₋₃heteroaryl^{a}, and —(C(H)(heteroaryl))-alkyl;

or wherein R₄ and R₅ together with the nitrogen atom to which they are attached form a carbon-containing 4-, 5-, 6-, or 7-membered heterocyclic ring which may be saturated or unsaturated with 1 or 2 double bonds, optionally containing one or two additional heteroatoms selected from N, O, and S, wherein the ring may be optionally substituted with 1, 2, or 3 substituents selected from oxo, alkyl, alkoxy, OH, halo and CF_{3;}
R₆ is selected from H, alkyl, —(CH₂)₀₋₃-cycloalkyl, —(CH₂)₀₋₃-heterocycloalkyl, —(CH(alkyl))-heterocycloalkyl, —(CH₂)₀₋₃-polycycloalkyl, —(CH₂)₀₋₃-aryl, —(C(H)(phenyl)-alkyl, —(CH₂)₀₋₃heteroaryl, and —(C(H)(heteroaryl))-alkyl;
R₁₄ is selected from H, alkyl, cycloalkyl, and heterocycloalkyl;
R₁₅ is selected from H, alkyl, —(CH₂)₀₋₃-cycloalkyl, —(CH₂)₀₋₃-heterocycloalkyl, —(CH(alkyl)-heterocycloalkyl, —(CH₂)₀₋₃-aryl, —(C(H)(phenyl))-alkyl, —(CH₂)₀₋₃heteroaryl^{a}, and —(C(H)(heteroaryl))-alkyl;
R₁₆ is selected from H, alkyl, cycloalkyl, and heterocycloalkyl; R₃₄ is selected from H, alkyl, cycloalkyl, and heterocycloalkyl;
R₃₅ is selected from H, alkyl, —(CH₂) 03-cycloalkyl, —(CH₂)₀₋₃-heterocycloalkyl, —(CH(alkyl)-heterocycloalkyl, —(CH₂)₀₋₃-aryl, —(C(H)(phenyl))-alkyl, —(CH₂)₀₋₃heteroaryl^{a}, and —(C(H)(heteroaryl))-alkyl;
R₁ is:
(i) Formula (II),

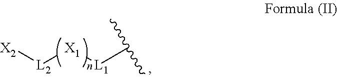

wherein:
n is 0 or 1;
L₁ is a linker selected from a single bond, —(CH₂)₁₋₃—, —(CH)(alkyl)-, and —C(alkyl)(alkyl);
X₁ is phenyl or a 5- or 6-membered carbon-containing aromatic ring containing one, two or three ring members that are selected from N, S, and O;
wherein X₁ may be optionally substituted with 1 or 2 substituents independently selected from alkyl, cycloalkyl, alkoxy, OH, CF₃, and halo;
L₂ is a linker selected from a single bond, —(CH₂)₁₋₃—, —(CH)(alkyl)-, —C(alkyl)(alkyl)-, —C(═O)—, and —SO₂—; and
X₂ is either:
(a) —NR₂₀R₂₁, wherein R₂₀ is selected from H, alkyl, and —(CH₂)₀₋₃—R_{22;}
wherein R₂₁ is selected from H, alkyl, and —(CH₂)₀₋₃—R₂₃;
wherein R₂₂ is selected from cycloalkyl, heterocycloalkyl, aryl, heteroaryl,

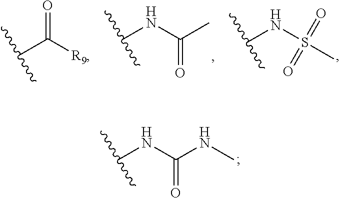

wherein R₂₃ is selected from cycloalkyl, heterocycloalkyl, aryl, heteroaryl,

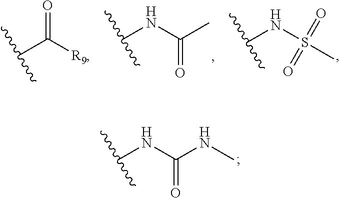

or
(b) 2-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and
   R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O; or,
(ii) Formula (III) comprising an aromatic ring A fused to a non-aromatic ring A′,

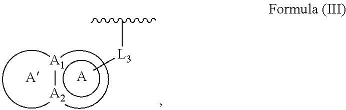

wherein:
L₃ is a linker selected from a single bond, —(CH₂)₁₋₃—, —(CH)(alkyl)-, and —C(alkyl)(alkyl)-;
A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, CF₃, and halo;
A′ is a non-aromatic carbon-containing monocyclic ring containing 5, 6, or 7, ring members, wherein at least one ring member is independently selected from N, S, and O; wherein A′ may be optionally substituted with one R₇, wherein R₇ is selected from alkyl, alkoxy, and —(CH₂)₀₋₃—R₈,
wherein R₈ is selected from cycloalkyl, heterocycloalkyl, aryl, heteroaryl,

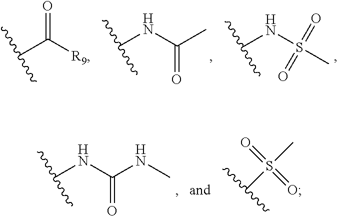

wherein R₉ is selected from H, alkyl, OH, NH₂, and alkoxy;
wherein A′ may be optionally substituted with 1, 2, or 3 further substituents R₁₀, R₁₁, and
R₁₇, wherein R₁₀, R₁₁, and R₁₇, are independently selected from alkyl, cycloalkyl, alkoxy, OH, oxo, CF₃, and halo,
or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O; and
R₃ is phenyl, or a 5- or 6-membered carbon-containing aromatic ring containing one, two or three ring members that are selected from N, S, and O, wherein R₃ is substituted with one or two substituents independently selected from small alkyl, small alkoxy, cyclopropyl, OH, CF₃, halo, —C(═O)—NH₂, —C(═O)—N(H)(small alkyl), —C(═O)—N(small alkyl)(small alkyl), and CN; and
alkyl is a linear saturated hydrocarbon having up to 10 carbon atoms (C₁-C₁₀) or a branched saturated hydrocarbon of between 3 and 10 carbon atoms (C₃-C₁₀); alkyl may optionally be substituted with 1, 2 or 3 substituents independently selected from cyclopropyl, (C₁-C₅) alkoxy, OH, —NR₁₂R₁₃, —C(═O) NR₁₂R₁₃, CN, CF₃, and halo;
small alkyl is a linear saturated hydrocarbon having up to 4 carbon atoms (C₁-C₄) or a branched saturated hydrocarbon of between 3 and 4 carbon atoms (C₃-C₄); small alkyl may optionally be substituted with 1 or 2 substituents independently selected from cyclopropyl, OH, CN, CF₃, NH₂, —C(═O)NH₂, and halo;
alkoxy is OCF₃, a linear O-linked hydrocarbon of between 1 and 6 carbon atoms (C₁-C₆), or a branched O-linked hydrocarbon of between 3 and 6 carbon atoms (C₃-Cs); alkoxy may optionally be substituted with 1 or 2 substituents independently selected from cyclopropyl, OH, CN, CF₃, and halo;
small alkoxy is OCF₃, a linear O-linked hydrocarbon of between 1 and 4 carbon atoms (C₁-C₄), or a branched O-linked hydrocarbon of between 3 and 4 carbon atoms (C₃-C₄); small alkoxy may optionally be substituted with 1 or 2 substituents independently selected from cyclopropyl, OH, CN, CF₃, and halo;
aryl is phenyl, biphenyl or naphthyl; aryl may be optionally substituted with 1, 2 or 3 substituents independently selected from alkyl, alkoxy, methylenedioxy, ethylenedioxy, OH, halo, CN, —(CH₂)₀₋₃—NR₁₂R₁₃, OCF₃ and CF_{3;}
polycycloalkyl is a bicyclic saturated hydrocarbon ring system of between 5 and 8 carbon atoms (C₅-C₈), wherein the bicyclic ring is bridged, or is adamantyl; polycycloalkyl may optionally be substituted with 1 or 2 substituents independently selected from alkyl, (C₁-C₆) alkoxy, OH, CN, CF₃, and halo;
cycloalkyl is a monocyclic saturated hydrocarbon ring of between 3 and 6 carbon atoms (C₃-C₆); cycloalkyl may optionally be substituted with 1 or 2 substituents independently selected from alkyl, (C₁-C₆) alkoxy, OH, CN, CF₃, and halo;
halo is F, Cl, Br, or I;
heteroaryl is a 5- or 6-membered carbon-containing aromatic ring containing one, two or three ring members that are selected from N, S, and O; wherein heteroaryl may be optionally substituted with 1, 2 or 3 substituents independently selected from alkyl, alkoxy, cyclopropyl, OH, halo, CN, and CF_{3;}
heteroaryl^{a} is a 5-, 6-, 9- or 10-membered mono- or bi-cyclic aromatic ring containing, where possible, 1, 2, 3 or 4 ring members independently selected from N, S, and O; wherein heteroaryl^{a} may be optionally substituted with 1, 2 or 3 substituents independently selected from alkyl, alkoxy, cyclopropyl, OH, halo, CN, and CF_{3;}
heterocycloalkyl is a non-aromatic carbon-containing mono- or bi-cyclic ring containing 3, 4, 5, 6, or 7 ring members, wherein at least one ring member is independently selected from N, S, and O; heterocycloalkyl may optionally be substituted with 1 or 2 substituents independently selected from alkyl, cyclopropyl, (C₁-C₆) alkoxy, OH, CN, CF₃, oxo, and halo; wherein when heterocycloalkyl is a bi-cyclic ring system, the bicyclic ring system is fused, bridged or spiro;
each R₁₂ and R₁₃ is independently selected from H, alkyl, cycloalkyl, and heterocycloalkyl;
and tautomers, isomers, stereoisomers (including enantiomers, diastereoisomers and racemic and scalemic mixtures thereof), deuterated analogues, and pharmaceutically acceptable salts and/or solvates thereof.

4. A compound of Formula (I),

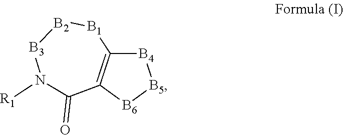

wherein:
B₁ is a carbon centre or nitrogen centre; and either:
(i) B₂ and B₃ together form a single bond between B₁ and the nitrogen atom to which R₁ is attached,
(ii) B₂ is a carbon centre or a nitrogen centre, and B₃ is a single bond between B₂ and the nitrogen atom to which R₁ is attached,
(iii) B₂ is a carbon centre and B₃ is a nitrogen centre, or
(iv) B₂ and B₃ are both carbon centres,
wherein, when B₂ is a carbon centre or nitrogen centre, B₁ is connected to B₂ via either a single or double bond;
wherein, when both B₂ and B₃ are present, B₂ and B₃ are connected via either a single or double bond;
wherein B₁ may, where possible, be optionally substituted with 1 or 2 substituents selected from alkyl, cycloalkyl, alkoxy, OH, CF₃, CN, and halo, or 1 oxo substituent, or 2 substituents which, together with B₁ to which they are both attached, form a 3-, 4-, or 5-membered non-aromatic monocyclic carbon-containing ring, optionally wherein one or two ring members are selected from N, S, and O;
wherein, when B₂ is a carbon centre, B₂ may be optionally substituted with 1 or 2 substituents selected from alkyl, cycloalkyl, alkoxy, OH, CF₃, CN, and halo, or 1 oxo substituent; and
wherein, when B₃ is a carbon centre, B₃ may be optionally substituted with 1 or 2 substituents selected from alkyl, cycloalkyl, alkoxy, OH, CF₃, CN, and halo, or 1 oxo substituent;
and either:
(i) B₄ is N(R₂), B₅ is N, and B₆ is C(R₃), and B₄ is connected to B₅ via a single bond and B₅ is connected to B₆ via a double bond;
(ii) B₄ is C(R₂), B₅ is N, and B₆ is N(R₃), and B₄ is connected to B₅ via a double bond and B₅ is connected to B₆ via a single bond; or
(iii) B₄ is C(R₂), B₅ is C(H), and B₆ is N(R₃), and B₄ is connected to B₅ via a double bond and B₅ is connected to B₆ via a single bond;
R₂ is selected from H, —(CH₂)₀₋₃—C(═O)—N(R₄)(R₅), —C(═O)—O—R₆, —(CH₂)₀₋₃—NR₁₄—C(═O)R₁₅, alkyl, cycloalkyl, heterocycloalkyl, alkoxy, —OH, —CF₃, halo, aryl, heteroaryl, —CH₂—O—R₁₆ and —CN;
wherein R₄ is H, alkyl, cycloalkyl, and heterocycloalkyl, and
R₅ is selected from alkyl, —(CH₂)₀₋₃-cycloalkyl, —(CH₂)₀₋₃-heterocycloalkyl, —(CH₂)₀₋₃-polycycloalkyl, —(CH₂)₀₋₃-aryl, —(C(H)(phenyl))-alkyl, —(CH₂)₀₋₃heteroaryl^{a}, and —(C(H)(heteroaryl))-alkyl;
or wherein R₄ and R₅ together with the nitrogen atom to which they are attached form a carbon-containing 4-, 5-, 6-, or 7-membered heterocyclic ring which may be saturated or unsaturated with 1 or 2 double bonds, optionally containing one or two additional heteroatoms selected from N, O, and S, wherein the ring may be optionally substituted with 1, 2, or 3 substituents selected from oxo, alkyl, alkoxy, OH, halo and CF_{3;}
R₆ is selected from H, alkyl, —(CH₂)₀₋₃-cycloalkyl, —(CH₂)₀₋₃-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, and —(C(H)(heteroaryl))-alkyl;
R₁₄ is selected from H, alkyl, cycloalkyl, and heterocycloalkyl;
R₁₅ is selected from H, alkyl, —(CH₂)₀₋₃-cycloalkyl, —(CH₂)₀₋₃-heterocycloalkyl, —(CH₂)₀₋₃-aryl, —(C(H)(phenyl))-alkyl, —(CH₂)₀₋₃heteroaryl^{a}, and —(C(H)(heteroaryl)-alkyl;
R₁₆ is selected from H, alkyl, cycloalkyl, and heterocycloalkyl;
R₁ is:
i) Formula (II),

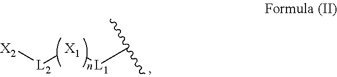

wherein:
n is 0 or 1;
L₁ is a linker selected from a single bond, —(CH₂)₁₋₃—, —(CH)(alkyl)-, and —C(alkyl)(alkyl)-; X₁ is phenyl or a 5- or 6-membered carbon-containing aromatic ring containing one, two or three ring members that are selected from N, S, and O;
wherein X₁ may be optionally substituted with 1 or 2 substituents independently selected from alkyl, cycloalkyl, alkoxy, OH, CF₃, and halo;
L₂ is a linker selected from a single bond, —(CH₂)₁₋₃—, —(CH)(alkyl)-, —C(alkyl)(alkyl)-, —C(═O)—, and —SO₂—; and
X₂ is either:
(a) —NR₂₀R₂₁,
wherein R₂₀ is selected from H, alkyl, and —(CH₂)₀₋₃—R_{22;}
wherein R₂₁ is selected from H, alkyl, and —(CH₂)₀₋₃—R₂₃;
wherein R₂₂ is selected from cycloalkyl, heterocycloalkyl, aryl, heteroaryl,

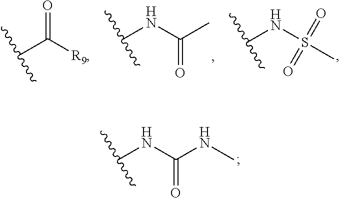

wherein R₂₃ is selected from cycloalkyl, heterocycloalkyl, aryl, heteroaryl,

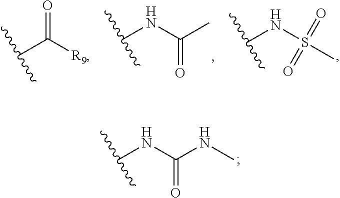

or
(b) 2-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected from alkyl, cycloalkyl, alkoxy, OH, oxo, CF₃, and halo,
   or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O; or,
(ii) Formula (III) comprising an aromatic ring A fused to a non-aromatic ring A′,

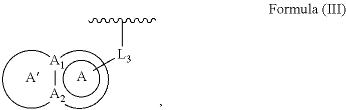

wherein:
L₃ is a linker selected from a single bond, —(CH₂)₁₋₃—, —(CH)(alkyl)-, and —C(alkyl)(alkyl)-;
A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, CF₃, and halo;
A′ is a non-aromatic carbon-containing monocyclic ring containing 5, 6, or 7, ring members, wherein at least one ring member is independently selected from N, S, and O; wherein A′ may be optionally substituted with one R₇, wherein R₇ is selected from alkyl, alkoxy, and —(CH₂)₀₋₃—R₈,
wherein R₈ is selected from cycloalkyl, heterocycloalkyl, aryl, heteroaryl,

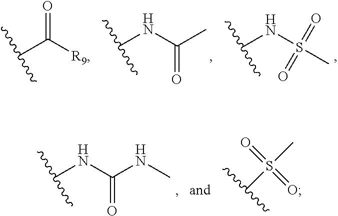

wherein R₉ is selected from H, alkyl, OH, NH₂, and alkoxy;
wherein A′ may be optionally substituted with 1 or 2 further substituents R₁₀ and R₁₁,
wherein R₁₀ and Ru are independently selected from alkyl, cycloalkyl, alkoxy, OH, oxo, CF₃, and halo,
or wherein A′ may be optionally substituted with 1 further substituent R₂₄ selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O; and
R₃ is phenyl, or a 5- or 6-membered carbon-containing aromatic ring containing one, two or three ring members that are selected from S, and O, N, wherein R₃ is substituted with one or two substituents independently selected from small alkyl, small alkoxy, cyclopropyl, OH, CF₃, halo, —C(═O)—NH₂, —C(═O)—N(H)(small alkyl), —C(═O)—N(small alkyl)(small alkyl), and CN; and
alkyl is a linear saturated hydrocarbon having up to 10 carbon atoms (C₁-C₁₀) or a branched saturated hydrocarbon of between 3 and 10 carbon atoms (C₃-C₁₀); alkyl may optionally be substituted with 1, 2 or 3 substituents independently selected from cyclopropyl, (C₁-C₆) alkoxy, OH, —NR₁₂R₁₃, —C(═O) NR₁₂R₁₃, CN, CF₃, and halo;
small alkyl is a linear saturated hydrocarbon having up to 4 carbon atoms (C₁-C₄) or a branched saturated hydrocarbon of between 3 and 4 carbon atoms (C₃-C₄); small alkyl may optionally be substituted with 1 or 2 substituents independently selected from cyclopropyl, OH, CN, CF₃, NH₂, —C(═O)NH₂, and halo;
alkoxy is OCF₃, a linear O-linked hydrocarbon of between 1 and 6 carbon atoms (C₁-C₆), or a branched O-linked hydrocarbon of between 3 and 6 carbon atoms (C₃-C₆); alkoxy may optionally be substituted with 1 or 2 substituents independently selected from cyclopropyl, OH, CN, CF₃, and halo;
small alkoxy is OCF₃, a linear O-linked hydrocarbon of between 1 and 4 carbon atoms (C₁-C₄), or a branched O-linked hydrocarbon of between 3 and 4 carbon atoms (C₃-C₄); small alkoxy may optionally be substituted with 1 or 2 substituents independently selected from cyclopropyl, OH, CN, CF₃, and halo;
aryl is phenyl, biphenyl or naphthyl; aryl may be optionally substituted with 1, 2 or 3 substituents independently selected from alkyl, alkoxy, methylenedioxy, ethylenedioxy, OH, halo, CN, —(CH₂)₀₋₃—NR₁₂R₁₃, OCF₃ and CF_{3;}
polycycloalkyl is a bicyclic saturated hydrocarbon ring system of between 5 and 8 carbon atoms (C₅-C₈), wherein the bicyclic ring is bridged, or is adamantyl; polycycloalkyl may optionally be substituted with 1 or 2 substituents independently selected from alkyl, (C₁-C₆) alkoxy, OH, CN, CF₃, and halo;
cycloalkyl is a monocyclic saturated hydrocarbon ring of between 3 and 6 carbon atoms (C₃-C₆); cycloalkyl may optionally be substituted with 1 or 2 substituents independently selected from alkyl, (C₁-C₆) alkoxy, OH, CN, CF₃, and halo;
halo is F, Cl, Br, or I;
heteroaryl is a 5- or 6-membered carbon-containing aromatic ring containing one, two or three ring members that are selected from N, S, and O; wherein heteroaryl may be optionally substituted with 1, 2 or 3 substituents independently selected from alkyl, alkoxy, cyclopropyl, OH, halo, CN, and CF_{3;}
heteroaryl^{a} is a 5-, 6-, 9- or 10-membered mono- or bi-cyclic aromatic ring containing, where possible, 1, 2, 3 or 4 ring members independently selected from N, S, and O; wherein heteroaryl^{a} may be optionally substituted with 1, 2 or 3 substituents independently selected from alkyl, alkoxy, cyclopropyl, OH, halo, CN, and CF_{3;}
heterocycloalkyl is a non-aromatic carbon-containing mono- or bi-cyclic ring containing 3, 4, 5, 6, or 7 ring members, wherein at least one ring member is independently selected from N, S, and O; heterocycloalkyl may optionally be substituted with 1 or 2 substituents independently selected from alkyl, cyclopropyl, (C₁-C₅) alkoxy, OH, CN, CF₃, oxo, and halo; wherein when heterocycloalkyl is a bi-cyclic ring system, the bicyclic ring system is fused, bridged or spiro;
each R₁₂ and R₁₃ is independently selected from H, alkyl, cycloalkyl, and heterocycloalkyl;
and tautomers, isomers, stereoisomers (including enantiomers, diastereoisomers and racemic and scalemic mixtures thereof), deuterated analogues, and pharmaceutically acceptable salts and/or solvates thereof.

5. A compound of Formula (I),

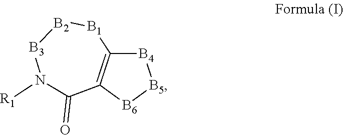

wherein:
B₁ is a carbon centre or nitrogen centre; and either:
(i) B₂ and B₃ together form a single bond between B₁ and the nitrogen atom to which R₁ is attached,
(ii) B₂ is a carbon centre or a nitrogen centre, and B₃ is a single bond between B₂ and the nitrogen atom to which R₁ is attached,
(iii) B₂ is a carbon centre and B₃ is a nitrogen centre, or
(iv) B₂ and B₃ are both carbon centres, wherein, when B₂ is a carbon centre or nitrogen centre, B₁ is connected to B₂ via either a single or double bond;
wherein, when both B₂ and B₃ are present, B₂ and B₃ are connected via either a single or double bond;
wherein B₁ may, where possible, be optionally substituted with 1 or 2 substituents selected from alkyl, cycloalkyl, alkoxy, OH, CF₃, CN, and halo, or 1 oxo substituent, or 2 substituents which, together with B₁ to which they are both attached, form a 3-, 4-, or 5-membered non-aromatic monocyclic carbon-containing ring, optionally wherein one or two ring members are selected from N, S, and O;
wherein, when B₂ is a carbon centre, B₂ may be optionally substituted with 1 or 2 substituents selected from alkyl, cycloalkyl, alkoxy, OH, CF₃, CN, and halo, or 1 oxo substituent; and
wherein, when B₃ is a carbon centre, B₃ may be optionally substituted with 1 or 2 substituents selected from alkyl, cycloalkyl, alkoxy, OH, CF₃, CN, and halo, or 1 oxo substituent;
and either:
(i) B₄ is N(R₂), B₅ is N, and B₆ is C(R₃), and B₄ is connected to B₅ via a single bond and B₅ is connected to B₆ via a double bond;
(ii) B₄ is C(R₂), B₅ is N, and B₆ is N(R₃), and B₄ is connected to B₅ via a double bond and B₅ is connected to B₆ via a single bond; or
(iii) B₄ is C(R₂), B₅ is C(H), and B₆ is N(R₃), and B₄ is connected to B₅ via a double bond and B₅ is connected to B₆ via a single bond;
R₂ is selected from H, —(CH₂)₀₋₃—C(═O)—N(R₄)(R₅), —C(═O)—O—R₆, —(CH₂)₀₋₃—NR₁₄—C(═O)R₁₅, alkyl, cycloalkyl, heterocycloalkyl, alkoxy, —OH, —CF₃, halo, aryl, heteroaryl, —CH₂—O—R₁₆ and —CN; wherein R₄ is H, alkyl, cycloalkyl, and heterocycloalkyl, and
R₅ is selected from alkyl, —(CH₂)₀₋₃-cycloalkyl, —(CH₂)₀₋₃-heterocycloalkyl, —(CH₂)₀₋₃-polycycloalkyl, —(CH₂)₀₋₃-aryl, —(C(H)(phenyl))-alkyl, —(CH₂)₀₋₃heteroaryl^{a}, and —(C(H)(heteroaryl))-alkyl;
or wherein R₄ and R₅ together with the nitrogen atom to which they are attached form a carbon-containing 4-, 5-, 6-, or 7-membered heterocyclic ring which may be saturated or unsaturated with 1 or 2 double bonds, optionally containing one or two additional heteroatoms selected from N, O, and S, wherein the ring may be optionally substituted with 1, 2, or 3 substituents selected from oxo, alkyl, alkoxy, OH, halo and CF_{3;}
R₆ is selected from H, alkyl, —(CH₂)₀₋₃-cycloalkyl, —(CH₂)₀₋₃-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, and —(C(H)(heteroaryl))-alkyl;
R₁₄ is selected from H, alkyl, cycloalkyl, and heterocycloalkyl;
R₁₅ is selected from H, alkyl, —(CH₂)₀₋₃-cycloalkyl, —(CH₂)₀₋₃-heterocycloalkyl, —(CH₂)₀₋₃-aryl, —(C(H)(phenyl))-alkyl, —(CH₂)₀₋₃heteroaryl^{a}, and —(C(H)(heteroaryl))-alkyl;
R₁₆ is selected from H, alkyl, cycloalkyl, and heterocycloalkyl;
R₁ is:
(i) Formula (II),

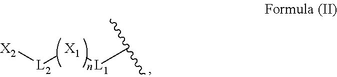

wherein:
n is 0 or 1;
L₁ is a linker selected from a single bond, —(CH₂)₁₋₃—, —(CH)(alkyl)-, and —C(alkyl)(alkyl);
X₁ is phenyl or a 5- or 6-membered carbon-containing aromatic ring containing one, two or three ring members that are selected from N, S, and O;
wherein X₁ may be optionally substituted with 1 or 2 substituents independently selected from alkyl, cycloalkyl, alkoxy, OH, CF₃, and halo;
L₂ is a linker selected from a single bond, —(CH₂)₁₋₃—, —(CH)(alkyl)-, —C(alkyl)(alkyl)-, —C(═O)—, and —SO₂—; and
X₂ is either:
(a) —NR₂₀R₂₁,
wherein R₂₀ is selected from H, alkyl, and —(CH₂)₀₋₃—R_{22;}
wherein R₂₁ is selected from H, alkyl, and —(CH₂)₀₋₃—R₂₃;
wherein R₂₂ is selected from cycloalkyl, heterocycloalkyl, aryl, heteroaryl,

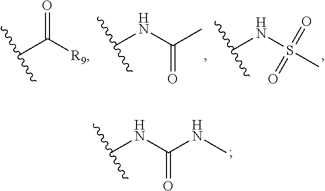

wherein R₂₃ is selected from cycloalkyl, heterocycloalkyl, aryl, heteroaryl,

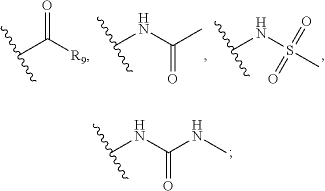

or
(b) 2-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O; or,
(ii) Formula (III) comprising an aromatic ring A fused to a non-aromatic ring A′,

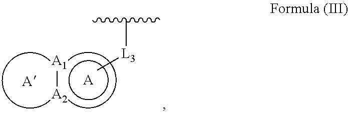

wherein:
L₃ is a linker selected from a single bond, —(CH₂)₁₋₃—, —(CH)(alkyl)-, and —C(alkyl)(alkyl)-;
A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, CF₃, and halo;
A′ is a non-aromatic carbon-containing monocyclic ring containing 5, 6, or 7, ring members, wherein at least one ring member is independently selected from N, S, and O; wherein A′ may be optionally substituted with one R₇, wherein R₇ is selected from alkyl, alkoxy, and —(CH₂)₀₋₃—R₈,
wherein R₈ is selected from cycloalkyl, heterocycloalkyl, aryl, heteroaryl,

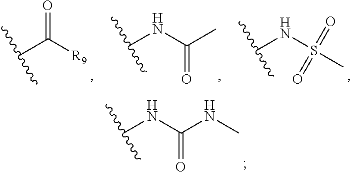

wherein R₉ is selected from H, alkyl, OH, NH₂, and alkoxy;
wherein A′ may be optionally substituted with 1 or 2 further substituents R₁₀ and R₁₁,
wherein R₁₀ and R₁₁ are independently selected from alkyl, cycloalkyl, alkoxy, OH, oxo, CF₃, and halo,
or wherein A′ may be optionally substituted with 1 further substituent R₂₄ selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O; and
R₃ is phenyl, or a 5- or 6-membered carbon-containing aromatic ring containing one, two or three ring members that are selected from N, S, and O, wherein R₃ is substituted with one or two substituents independently selected from small alkyl, small alkoxy, cyclopropyl, OH, CF₃, halo, —C(═O)—NH₂, —C(═O)—N(H)(small alkyl), —C(═O)—N(small alkyl)(small alkyl), and CN; and
alkyl is a linear saturated hydrocarbon having up to 10 carbon atoms (C₁-C₁₀) or a branched saturated hydrocarbon of between 3 and 10 carbon atoms (C₃-C₁₀); alkyl may optionally be substituted with 1, 2 or 3 substituents independently selected from cyclopropyl, (C₁-C₆) alkoxy, OH, —NR₁₂R₁₃, —C(═O) NR₁₂R₁₃, CN, CF₃, and halo;
small alkyl is a linear saturated hydrocarbon having up to 4 carbon atoms (C₁-C₄) or a branched saturated hydrocarbon of between 3 and 4 carbon atoms (C₃-C₄); small alkyl may optionally be substituted with 1 or 2 substituents independently selected from cyclopropyl, OH, CN, CF₃, NH₂, —C(═O)NH₂, and halo;
alkoxy is OCF₃, a linear O-linked hydrocarbon of between 1 and 6 carbon atoms (C₁-C₆), or a branched O-linked hydrocarbon of between 3 and 6 carbon atoms (C₃-C₆); alkoxy may optionally be substituted with 1 or 2 substituents independently selected from cyclopropyl, OH, CN, CF₃, and halo;
small alkoxy is OCF₃, a linear O-linked hydrocarbon of between 1 and 4 carbon atoms (C₁-C₄), or a branched O-linked hydrocarbon of between 3 and 4 carbon atoms (C₃-C₄); small alkoxy may optionally be substituted with 1 or 2 substituents independently selected from cyclopropyl, OH, CN, CF₃, and halo;
aryl is phenyl, biphenyl or naphthyl; aryl may be optionally substituted with 1, 2 or 3 substituents independently selected from alkyl, alkoxy, methylenedioxy, ethylenedioxy, OH, halo, CN, —(CH₂)₀₋₃—NR₁₂R₁₃, OCF₃ and CF_{3;}
polycycloalkyl is a bicyclic saturated hydrocarbon ring system of between 5 and 8 carbon atoms (C₅-C₈), wherein the bicyclic ring is bridged, or is adamantyl; polycycloalkyl may optionally be substituted with 1 or 2 substituents independently selected from alkyl, (C₁-C₆) alkoxy, OH, CN, CF₃, and halo;
cycloalkyl is a monocyclic saturated hydrocarbon ring of between 3 and 6 carbon atoms (C₃-C₆); cycloalkyl may optionally be substituted with 1 or 2 substituents independently selected from
halo is F, Cl, Br, or I;
heteroaryl is a 5- or 6-membered carbon-containing aromatic ring containing one, two or three ring members that are selected from N, S, and O; wherein heteroaryl may be optionally substituted with 1, 2 or 3 substituents independently selected from alkyl, alkoxy, OH, halo, CN, and CF_{3;}
heteroaryl^{a} is a 5-, 6-, 9- or 10-membered mono- or bi-cyclic aromatic ring containing, where possible, 1, 2, 3 or 4 ring members independently selected from N, S, and O; wherein heteroaryl^{a} may be optionally substituted with 1, 2 or 3 substituents independently selected from alkyl, alkoxy, OH, halo, CN, and CF_{3;}
heterocycloalkyl is a non-aromatic carbon-containing mono- or bi-cyclic ring containing 3, 4, 5, 6, or 7 ring members, wherein at least one ring member is independently selected from N, S, and O; heterocycloalkyl may optionally be substituted with 1 or 2 substituents independently selected from alkyl, cyclopropyl, (C₁-C₅) alkoxy, OH, CN, CF₃, oxo, and halo; wherein when heterocycloalkyl is a bi-cyclic ring system, the bicyclic ring system is fused, bridged or spiro;
each R₁₂ and R₁₃ is independently selected from H, alkyl, cycloalkyl, and heterocycloalkyl;
and tautomers, isomers, stereoisomers (including enantiomers, diastereoisomers and racemic and scalemic mixtures thereof), deuterated analogues, and pharmaceutically acceptable salts and/or solvates thereof.

6. A compound of Formula (I),

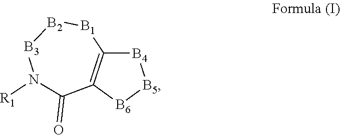

wherein:
B₁ is a carbon centre or nitrogen centre; and either:
(i) B₂ and B₃ together form a single bond between B₁ and the nitrogen atom to which R₁ is attached,
(ii) B₂ is a carbon centre or a nitrogen centre, and B₃ is a single bond between B₂ and the nitrogen atom to which R₁ is attached,
wherein, when B₂ is a carbon centre or nitrogen centre, B₁ is connected to B₂ via either a single or double bond;
wherein B₁ may, where possible, be optionally substituted with 1 or 2 substituents selected from alkyl, cycloalkyl, alkoxy, OH, CF₃, CN, and halo, or 1 oxo substituent, or 2 substituents which, together with B₁ to which they are both attached, form a 3-, 4-, or 5-membered non-aromatic monocyclic carbon-containing ring, optionally wherein one or two ring members are selected from N, S, and O;
wherein, when B₂ is a carbon centre, B₂ may be optionally substituted with 1 or 2 substituents selected from alkyl, cycloalkyl, alkoxy, OH, CF₃, CN, and halo, or 1 oxo substituent;
and either:
(i) B₄ is N(R₂), B₅ is N, and B₆ is C(R₃), and B₄ is connected to B₅ via a single bond and B₅ is connected to B₆ via a double bond;
(ii) B₄ is C(R₂), B₅ is N, and B₆ is N(R₃), and B₄ is connected to B₅ via a double bond and B₅ is connected to B₆ via a single bond; or
(iii) B₄ is C(R₂), B₅ is C(H), and B₆ is N(R₃), and B₄ is connected to B₅ via a double bond and B₅ is connected to B₆ via a single bond;
R₂ is selected from H, —(CH₂)₀₋₃—C(═O)—N(R₄)(R₅), —C(═O)—O—R₆, —(CH₂)₀₋₃—NR₁₄—C(═O)R₁₅, —(CH₂)₀₋₃—NR₃₄—S(═O)₂R₃₅, alkyl, cycloalkyl, heterocycloalkyl, alkoxy, —OH, —CF₃, halo, aryl, heteroaryl, —CH₂—O—R₁₆ and —CN;
wherein R₄ is H, alkyl, cycloalkyl, and heterocycloalkyl, and
R₅ is selected from alkyl, —OH, —S(═O)₂-(small alkyl), —(CH₂)₁₋₃—(C═O)—OR₃₃, —(CH₂)₁₋₃—(C═O)—N(R₃₆)(R₃₇), —(CH₂)₀₋₃-cycloalkyl^{b}, —(CH₂)₀₋₃-heterocycloalkyl, —(CH(alkyl))-heterocycloalkyl, —(CH₂)₀₋₃-polycycloalkyl, —(CH₂)₀₋₃-aryl, —(C(H)(phenyl))-alkyl, —(CH₂)₀₋₃heteroaryl^{a}, —(C(H)(heteroaryl))-alkyl, —(C(H)(alkyl))-(C═O)—OR₃₈, —(C(H)(alkyl))-(C═O)—N(R₃₉)(R₄₀), —(C(H)(heteroaryl))-(C═O)—OR₂₈, and —(C(H)(heteroaryl))-(C═O)—N(R₄₁)(R₄₂);
or wherein R₄ and R₅ together with the nitrogen atom to which they are attached form a carbon-containing 4-, 5-, 6-, or 7-membered heterocyclic ring which may be saturated or unsaturated with 1 or 2 double bonds, optionally containing one or two additional heteroatoms selected from N, O, and S, wherein the ring may be optionally substituted with 1, 2, or 3 substituents selected from oxo, alkyl, alkoxy, OH, halo and CF_{3;}
R₂₈, R₃₃ R₃₆, R₃₇, R₃₈, R₃₉, R₄₀, R₄₁, and R₄₂ are each independently selected from H and small alkyl; R₆ is selected from H, alkyl, —(CH₂)₀₋₃-cycloalkyl, —(CH₂)₀₋₃-heterocycloalkyl, —(CH(alkyl))-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, and —(C(H)(heteroaryl))-alkyl;
R₁₄ is selected from H, alkyl, cycloalkyl, and heterocycloalkyl;
R₁₅ is selected from H, alkyl, —(CH₂)₀₋₃-cycloalkyl, —(CH₂)₀₋₃-heterocycloalkyl, —(CH(alkyl))-heterocycloalkyl, —(CH₂)₀₋₃-aryl, —(C(H)(phenyl))-alkyl, —(CH₂)₀₋₃heteroaryl^{a}, and —(C(H)(heteroaryl))-alkyl;
R₁₆ is selected from H, alkyl, cycloalkyl, —(CH₂)₁₋₃—(C═O)—OH, heterocycloalkyl, and —(CH₂)₀₋₃heteroaryl;
R₃₄ is selected from H, alkyl, cycloalkyl, and heterocycloalkyl;
R₃₅ is selected from H, alkyl, —(CH₂)₀₋₃-cycloalkyl, —(CH₂)₀₋₃-heterocycloalkyl, —(CH(alkyl))-heterocycloalkyl, —(CH₂)₀₋₃-aryl, —(C(H)(phenyl))-alkyl, —(CH₂)₀₋₃heteroaryl^{a}, and —(C(H)(heteroaryl))-alkyl;
R₁ is:
i) Formula (II), wherein Formula (II) is represented by Formula (IIb),

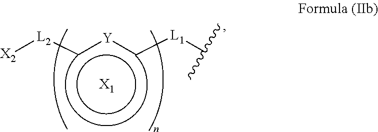

wherein:
n is 1;
L₁ is a linker selected from a single bond, —(CH₂)₁₋₃—, —(CH)(alkyl)-, and —C(alkyl)(alkyl)-;
Y is selected from C, N, O and S;
X₁ is phenyl or a 5- or 6-membered carbon-containing aromatic ring containing one, two or three ring members that are selected from N, S, and O;
wherein X₁ may be optionally substituted with 1 or 2 substituents independently selected from alkyl, cycloalkyl, alkoxy, OH, O(cyclopropyl), CF₃, and halo;
L₂ is a linker selected from a single bond, —(CH₂)₁₋₃—, —(CH)(alkyl)-, —C(alkyl)(alkyl)-, —C(═O)—, and —SO₂—; and
X₂ is either:
wherein R₂₀ is selected from H, alkyl, and —(CH₂)₀₋₃—R_{22;}
wherein R₂₁ is selected from H, alkyl, and —(CH₂)₀₋₃—R_{23;}
(a) —NR₂₀R₂₁,

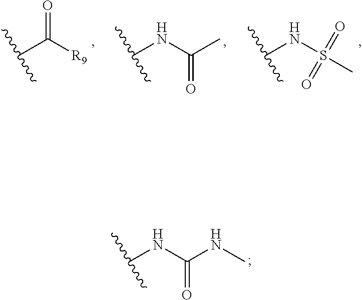

wherein R₂₂ is selected from cycloalkyl, heterocycloalkyl, aryl, heteroaryl,

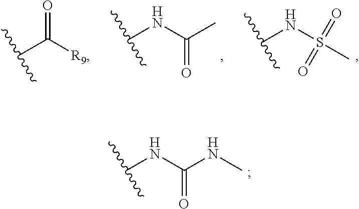

wherein R₂₃ is selected from cycloalkyl, heterocycloalkyl, aryl, heteroaryl, or
(b) 2-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O, and X₂ may be further optionally substituted with 1 or 2 substituents R₂₉ and R₃₀, wherein R₂₉ and R₃₀ are independently selected from alkyl, cycloalkyl, alkoxy, OH, oxo, CF₃, and halo; or,
(ii) Formula (III) comprising an aromatic ring A fused to a non-aromatic ring A′,

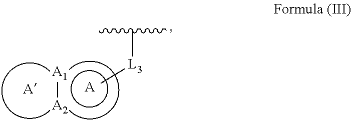

wherein:
L₃ is a linker selected from a single bond, —(CH₂)₁₋₃—, —(CH)(alkyl)-, and —C(alkyl)(alkyl)-;
A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, O(cyclopropyl), CF₃, and halo;
A′ is a non-aromatic carbon-containing monocyclic ring containing 5, 6, or 7, ring members, wherein at least one ring member is independently selected from N, S, and O; wherein A′ may be optionally substituted with one R₇, wherein R₇ is selected from alkyl, alkoxy, and —(CH₂)₀₋₃—R₈,
wherein R₈ is selected from cycloalkyl, heterocycloalkyl, aryl, heteroaryl,

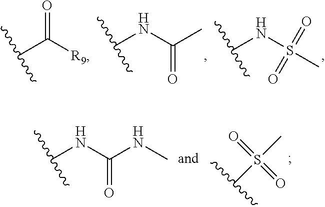

wherein R₉ is selected from H, alkyl, OH, N (R₃₁)(R₃₂), and alkoxy;
wherein R₃₁ is H or small alkyl;
wherein R₃₂ is selected from H, small alkyl, and —(CH₂)₀₋₃heteroaryl;
wherein A′ may be optionally substituted with 1, 2, or 3 further substituents R₁₀, R₁₁, and
R₁₇, wherein R₁₀, R₁₁, and R₁₇, are independently selected from alkyl, cycloalkyl, alkoxy, OH, oxo, CF₃, and halo, or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O,
or wherein A′ may be optionally substituted with 1 or 2 further substituents R₄₃ and R₄₄ independently selected from alkyl, cycloalkyl, alkoxy, OH, oxo, CF₃, and halo, and two different carbon ring members within A′ are connected by a bridging —CH₂-group; and
R₃ is phenyl, or a 5- or 6-membered carbon-containing aromatic ring containing one, two or three ring members that are selected from N, S and O, wherein R₃ is substituted with one, two, or three substituents independently selected from small alkyl^{b}, small alkoxy, cyclopropyl, OH, NH₂, CF₃, halo, —C(═O)—NH₂,

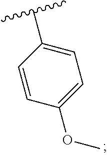 [SMILES: *c1ccc(OC)cc1]

—C(═O)—N(H)(small alkyl), —C(═O)—N(small alkyl)(small alkyl), and CN; wherein R₃ is not and
alkyl is a linear saturated hydrocarbon having up to 10 carbon atoms (C₁-C₁₀) or a branched saturated hydrocarbon of between 3 and 10 carbon atoms (C₃-C₁₀); alkyl may optionally be substituted with 1, 2 or 3 substituents independently selected from cyclopropyl, (C₁-C₆) alkoxy, OH, —NR₁₂R₁₃, —C(═O) NR₁₂R₁₃, CN, CF₃, and halo;
small alkyl is a linear saturated hydrocarbon having up to 4 carbon atoms (C₁-C₄) or a branched saturated hydrocarbon of between 3 and 4 carbon atoms (C₃-C₄); small alkyl may optionally be substituted with 1 or 2 substituents independently selected from cyclopropyl, OH, CN, CF₃, NH₂, —C(═O)NH₂, and halo;
small alkyl^{b} is a linear saturated hydrocarbon having up to 4 carbon atoms (C₁-C₄) or a branched saturated hydrocarbon of between 3 and 4 carbon atoms (C₃-C₄); small alkyl may optionally be substituted with 1 or 2 substituents independently selected from cyclopropyl, OH, OMe, OEt, OⁱPr, OⁿPr, O(cyclopropyl), OCF₃, OCHF₂, CN, CF₃, NH₂, —C(═O)NH₂, and halo;
alkoxy is OCF₃, O(cyclopropyl), a linear O-linked hydrocarbon of between 1 and 6 carbon atoms (C₁-C₆), or a branched O-linked hydrocarbon of between 3 and 6 carbon atoms (C₃-C₆); alkoxy may optionally be substituted with 1 or 2 substituents independently selected from cyclopropyl, OH, CN, CF₃, and halo;
small alkoxy is OCF₃, O(cyclopropyl), a linear O-linked hydrocarbon of between 1 and 4 carbon atoms (C₁-C₄), or a branched O-linked hydrocarbon of between 3 and 4 carbon atoms (C₃-C₄); small alkoxy may optionally be substituted with 1 or 2 substituents independently selected from cyclopropyl, OH, CN, CF₃, and halo;
aryl is phenyl, biphenyl or naphthyl; aryl may be optionally substituted with 1, 2 or 3 substituents independently selected from alkyl, alkoxy, methylenedioxy, ethylenedioxy, OH, halo, CN, —(CH₂)₀₋₃—NR₁₂R₁₃, OCF₃ and CF_{3;}
polycycloalkyl is a bicyclic saturated hydrocarbon ring system of between 5 and 8 carbon atoms (C₅-C₈), wherein the bicyclic ring is bridged, or is adamantyl; polycycloalkyl may optionally be substituted with 1 or 2 substituents independently selected from alkyl, (C₁-C) alkoxy, OH, CN, CF₃, and halo;
cycloalkyl is a monocyclic saturated hydrocarbon ring of between 3 and 6 carbon atoms (C₃-C₆); cycloalkyl may optionally be substituted with 1 or 2 substituents independently selected from alkyl, (C₁-C₆) alkoxy, OH, CN, CF₃, and halo
cycloalkyl^{a} is a monocyclic saturated hydrocarbon ring of between 3 and 6 carbon atoms (C₃-C₆); cycloalkyl may optionally be substituted with 1 or 2 substituents independently selected from alkyl, (C₁-C₆) alkoxy, OH, CN, CF₃, halo, and —(C═O)—OH;
cycloalkyl^{b} is a monocyclic saturated hydrocarbon ring of between 3 and 6 carbon atoms (C₃-C₆); cycloalkyl may optionally be substituted with 1 or 2 substituents independently selected from alkyl, (C₁-C₆) alkoxy, OH, NH₂, NH(small alkyl), CN, CF₃, halo, and —(C═O)—OH;
halo is F, Cl, Br, or I;
heteroaryl is a 5- or 6-membered carbon-containing aromatic ring containing one, two or three ring members that are selected from N, S, and O; wherein heteroaryl may be optionally substituted with 1, 2 or 3 substituents independently selected from alkyl, alkoxy, cyclopropyl, OH, halo, CN, and CF_{3;}
heteroaryl^{a} is a 5-, 6-, 9- or 10-membered mono- or bi-cyclic aromatic ring containing, where possible, 1, 2, 3 or 4 ring members independently selected from N, S, and O; wherein heteroaryl^{a} may be optionally substituted with 1, 2 or 3 substituents independently selected from alkyl, alkoxy, cyclopropyl, OH, halo, CN, and CF_{3;}
heterocycloalkyl is a non-aromatic carbon-containing mono- or bi-cyclic ring containing 3, 4, 5, 6, or 7 ring members, wherein at least one ring member is independently selected from N, S, and O; heterocycloalkyl may optionally be substituted with 1 or 2 substituents independently selected from alkyl, cyclopropyl, (C₁-C₅) alkoxy, OH, CN, CF₃, oxo, and halo; wherein when heterocycloalkyl is a bi-cyclic ring system, the bicyclic ring system is fused, bridged or spiro;
each R₁₂ and R₁₃ is independently selected from H, alkyl, cycloalkyl, and heterocycloalkyl;
and tautomers, isomers, stereoisomers (including enantiomers, diastereoisomers and racemic and scalemic mixtures thereof), deuterated analogues, and pharmaceutically acceptable salts and/or solvates thereof.

7. A compound of Formula (I),

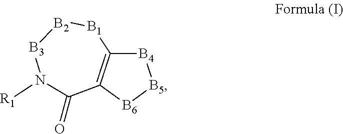

wherein:
B₁ is a carbon centre or nitrogen centre; and either:
i) B₂ and B₃ together form a single bond between B₁ and the nitrogen atom to which R₁ is attached,
(ii) B₂ is a carbon centre or a nitrogen centre, and B₃ is a single bond between B₂ and the nitrogen atom to which R₁ is attached,
(iii) B₂ is a carbon centre and B₃ is a nitrogen centre, or
(iv) B₂ and B₃ are both carbon centres,
wherein, when B₂ is a carbon centre or nitrogen centre, B₁ is connected to B₂ via either a single or double bond;
wherein, when both B₂ and B₃ are present, B₂ and B₃ are connected via either a single or double bond;
wherein B₁ may, where possible, be optionally substituted with 1 or 2 substituents selected from alkyl, cycloalkyl, alkoxy, OH, CF₃, CN, and halo, or 1 oxo substituent, or 2 substituents which, together with B₁ to which they are both attached, form a 3-, 4-, or 5-membered non-aromatic monocyclic carbon-containing ring, optionally wherein one or two ring members are selected from N, S, and O;
wherein, when B₂ is a carbon centre, B₂ may be optionally substituted with 1 or 2 substituents selected from alkyl, cycloalkyl, alkoxy, OH, CF₃, CN, and halo, or 1 oxo substituent; and
wherein, when B₃ is a carbon centre, B₃ may be optionally substituted with 1 or 2 substituents selected from alkyl, cycloalkyl, alkoxy, OH, CF₃, CN, and halo, or 1 oxo substituent;
and either:
(i) B₄ is N(R₂), B₅ is N, and B₆ is C(R₃), and B₄ is connected to B₅ via a single bond and B₅ is connected to B₆ via a double bond;
(ii) B₄ is C(R₂), B₅ is N, and B₆ is N(R₃), and B₄ is connected to B₅ via a double bond and B₅ is connected to B₆ via a single bond; or
(iii) B₄ is C(R₂), B₅ is C(H), and B₆ is N(R₃), and B₄ is connected to B₅ via a double bond and B₅ is connected to B₆ via a single bond;
R₂ is selected from H, —(CH₂)₀₋₃—C(═O)—N(R₄)(R₅), —C(═O)—O—R₆, —(CH₂)₀₋₃—NR₁₄—C(═O)R₁₅, —(CH₂)₀₋₃—NR₃₄—S(═O)₂R₃₅, alkyl, cycloalkyl, heterocycloalkyl, alkoxy, —OH, —CF₃, halo, aryl, heteroaryl, —CH₂—O—R₁₆ and —CN;
wherein R₄ is H, alkyl, cycloalkyl, and heterocycloalkyl, and
R₅ is selected from alkyl, —OH, —S(═O)₂-(small alkyl), —(CH₂)₁₋₃—(C═O)—OR₃₃, —(CH₂)₁₋₃—(C═O)—N(R₃₆)(R₃₇), —(CH₂)₀₋₃-cycloalkyl^{a}, —(CH₂)₀₋₃-heterocycloalkyl, —(CH₂)₀₋₃-polycycloalkyl, —(CH₂)₀₋₃-aryl, —(C(H)(phenyl))-alkyl, —(CH(alkyl))-heterocycloalkyl, —(CH₂)₀₋₃heteroaryl^{a}, —(C(H)(heteroaryl))-alkyl, —(C(H)(alkyl))-(C═O)—OR₃₈, —(C(H)(alkyl))-(C═O)—N(R₃₉)(R₄₀), —(C(H)(heteroaryl))-(C═O)—OR₂₈, and —(C(H)(heteroaryl))-(C═O)—N(R₄₁)(R₄₂);
or wherein R₄ and R₅ together with the nitrogen atom to which they are attached form a carbon-containing 4-, 5-, 6-, or 7-membered heterocyclic ring which may be saturated or unsaturated with 1 or 2 double bonds, optionally containing one or two additional heteroatoms selected from N, O, and S, wherein the ring may be optionally substituted with 1, 2, or 3 substituents selected from oxo, alkyl, alkoxy, OH, halo and CF_{3;}
R₂₈, R₃₃ R₃₆, R₃₇, R₃₈, R₃₉, R₄₀, R₄₁, and R₄₂ are each independently selected from H and small alkyl; R₆ is selected from H, alkyl, —(CH₂)₀₋₃-cycloalkyl, —(CH₂)₀₋₃-heterocycloalkyl, —(CH(alkyl))-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, and —(C(H)(heteroaryl))-alkyl;
R₁₄ is selected from H, alkyl, cycloalkyl, and heterocycloalkyl;
R₁₅ is selected from H, alkyl, —(CH₂)₀₋₃-cycloalkyl, —(CH₂)₀₋₃-heterocycloalkyl, —(CH(alkyl))-heterocycloalkyl, —(CH₂)₀₋₃-aryl, —(C(H)(phenyl))-alkyl, —(CH₂)₀₋₃heteroaryl^{a}, and —(C(H)(heteroaryl))-alkyl;
R₁₆ is selected from H, alkyl, cycloalkyl, —(CH₂)₁₋₃—(C═O)—OH, heterocycloalkyl, and —(CH₂)₀₋₃heteroaryl;
R₃₄ is selected from H, alkyl, cycloalkyl, and heterocycloalkyl;
R₃₅ is selected from H, alkyl, —(CH₂)₀₋₃-cycloalkyl, —(CH₂)₀₋₃-heterocycloalkyl, —(CH(alkyl))-heterocycloalkyl, —(CH₂)₀₋₃-aryl, —(C(H)(phenyl))-alkyl, —(CH₂)₀₋₃heteroaryl^{a}, and —(C(H)(heteroaryl))-alkyl;
R₁ is:
(i) Formula (II), wherein Formula (II) is represented by Formula (IIb),

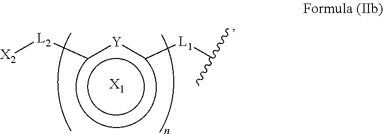

wherein:
n is 1;
L₁ is a linker selected from a single bond, —(CH₂)₁₋₃—, —(CH)(alkyl)-, and —C(alkyl)(alkyl)-;
Y is selected from C, N, O and S;
X₁ is phenyl or a 5- or 6-membered carbon-containing aromatic ring containing one, two or three ring members that are selected from N, S, and O;
wherein X₁ may be optionally substituted with 1 or 2 substituents independently selected from alkyl, cycloalkyl, alkoxy, OH, CF₃, and halo;
L₂ is a linker selected from a single bond, —(CH₂)₁₋₃—, —(CH)(alkyl)-, —C(alkyl)(alkyl)-, —C(═O)—, and —SO₂—; and
X₂ is either:
(a) —NR₂₀R₂₁,
wherein R₂₀ is selected from H, alkyl, and —(CH₂)₀₋₃—R_{22;}
wherein R₂₁ is selected from H, alkyl, and —(CH₂)₀₋₃—R₂₃;

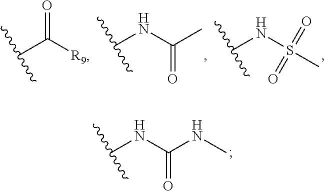

wherein R₂₂ is selected from cycloalkyl, heterocycloalkyl, aryl, heteroaryl,

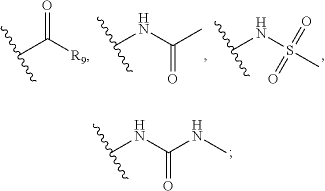

wherein R₂₃ is selected from cycloalkyl, heterocycloalkyl, aryl, heteroaryl, or
(b) 2-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O, and X₂ may be further optionally substituted with 1 or 2 substituents R₂₉ and R₃₀, wherein R₂₉ and R₃₀ are independently selected from alkyl, cycloalkyl, alkoxy, OH, oxo, CF₃, and halo; or,
(ii) Formula (III) comprising an aromatic ring A fused to a non-aromatic ring A′,

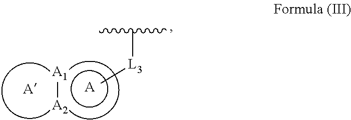

wherein:
L₃ is a linker selected from a single bond, —(CH₂)₁₋₃—, —(CH)(alkyl)-, and —C(alkyl)(alkyl)-;
A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, CF₃, and halo;
A′ is a non-aromatic carbon-containing monocyclic ring containing 5, 6, or 7, ring members, wherein at least one ring member is independently selected from N, S, and O; wherein A′ may be optionally substituted with one R₇, wherein R₇ is selected from alkyl, alkoxy, and —(CH₂)₀₋₃—R₈,
wherein R₅ is selected from cycloalkyl, heterocycloalkyl, aryl, heteroaryl,

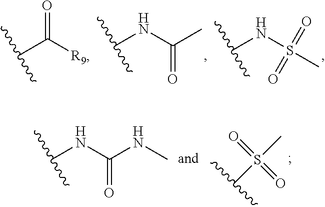

wherein R₉ is selected from H, alkyl, OH, N (R₃₁)(R₃₂), and alkoxy;
wherein R₃₁ is H or small alkyl;
wherein R₃₂ is selected from H, small alkyl, and —(CH₂)₀₋₃heteroaryl;
wherein A′ may be optionally substituted with 1, 2, or 3 further substituents R₁₀, R₁₁, and
R₁₇, wherein R₁₀, R₁₁, and R₁₇, are independently selected from alkyl, cycloalkyl, alkoxy, OH, oxo, CF₃, and halo, or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O; and
R₃ is phenyl, or a 5- or 6-membered carbon-containing aromatic ring containing one, two or three ring members that are selected N, S, and O, from wherein R₃ is substituted with one or two substituents independently selected from small alkyl, small alkoxy, cyclopropyl, OH, CF₃, halo, —C(═O)—NH₂, —C(═O)—N(H)(small alkyl), —C(═O)—N(small alkyl)(small alkyl), and CN; wherein R₃ is not

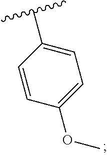 [SMILES: *c1ccc(OC)cc1]

and
alkyl is a linear saturated hydrocarbon having up to 10 carbon atoms (C₁-C₁₀) or a branched saturated hydrocarbon of between 3 and 10 carbon atoms (C₃-C₁₀); alkyl may optionally be substituted with 1, 2 or 3 substituents independently selected from cyclopropyl, (C₁-C₅) alkoxy, OH, —NR₁₂R₁₃, —C(═O) NR₁₂R₁₃, CN, CF₃, and halo;
small alkyl is a linear saturated hydrocarbon having up to 4 carbon atoms (C₁-C₄) or a branched saturated hydrocarbon of between 3 and 4 carbon atoms (C₃-C₄); small alkyl may optionally be substituted with 1 or 2 substituents independently selected from cyclopropyl, OH, CN, CF₃, NH₂, —C(═O)NH₂, and halo;
alkoxy is OCF₃, a linear O-linked hydrocarbon of between 1 and 6 carbon atoms (C₁-C₆), or a branched O-linked hydrocarbon of between 3 and 6 carbon atoms (C₃-C₆); alkoxy may optionally be substituted with 1 or 2 substituents independently selected from cyclopropyl, OH, CN, CF₃, and halo;
small alkoxy is OCF₃, a linear O-linked hydrocarbon of between 1 and 4 carbon atoms (C₁-C₄), or a branched O-linked hydrocarbon of between 3 and 4 carbon atoms (C₃-C₄); small alkoxy may optionally be substituted with 1 or 2 substituents independently selected from cyclopropyl, OH, CN, CF₃, and halo;
aryl is phenyl, biphenyl or naphthyl; aryl may be optionally substituted with 1, 2 or 3 substituents independently selected from alkyl, alkoxy, methylenedioxy, ethylenedioxy, OH, halo, CN, —(CH₂)₀₋₃—NR₁₂R₁₃, OCF₃ and CF_{3;}
polycycloalkyl is a bicyclic saturated hydrocarbon ring system of between 5 and 8 carbon atoms (C₅-C₈), wherein the bicyclic ring is bridged, or is adamantyl; polycycloalkyl may optionally be substituted with 1 or 2 substituents independently selected from alkyl, (C₁-C₆) alkoxy, OH, CN, CF₃, and halo;
cycloalkyl is a monocyclic saturated hydrocarbon ring of between 3 and 6 carbon atoms (C₃-C₆); cycloalkyl may optionally be substituted with 1 or 2 substituents independently selected from alkyl, (C₁-C₆) alkoxy, OH, CN, CF₃, and halo
cycloalkyl^{a} is a monocyclic saturated hydrocarbon ring of between 3 and 6 carbon atoms (C₃-C₆); cycloalkyl may optionally be substituted with 1 or 2 substituents independently selected from alkyl, (C₁-C₆) alkoxy, OH, CN, CF₃, halo, and —(C═O)—OH;
halo is F, Cl, Br, or I;
heteroaryl is a 5- or 6-membered carbon-containing aromatic ring containing one, two or three ring members that are selected from N, S, and O; wherein heteroaryl may be optionally substituted with 1, 2 or 3 substituents independently selected from alkyl, alkoxy, cyclopropyl, OH, halo, CN, and CF_{3;}
heteroaryl^{a} is a 5-, 6-, 9- or 10-membered mono- or bi-cyclic aromatic ring containing, where possible, 1, 2, 3 or 4 ring members independently selected from N, S, and O; wherein heteroaryl^{a} may be optionally substituted with 1, 2 or 3 substituents independently selected from alkyl, alkoxy, cyclopropyl, OH, halo, CN, and CF_{3;}
heterocycloalkyl is a non-aromatic carbon-containing mono- or bi-cyclic ring containing 3, 4, 5, 6, or 7 ring members, wherein at least one ring member is independently selected from N, S, and O; heterocycloalkyl may optionally be substituted with 1 or 2 substituents independently selected from alkyl, cyclopropyl, (C₁-C₆) alkoxy, OH, CN, CF₃, oxo, and halo; wherein when heterocycloalkyl is a bi-cyclic ring system, the bicyclic ring system is fused, bridged or spiro;
each R₁₂ and R₁₃ is independently selected from H, alkyl, cycloalkyl, and heterocycloalkyl;
and tautomers, isomers, stereoisomers (including enantiomers, diastereoisomers and racemic and scalemic mixtures thereof), deuterated analogues, and pharmaceutically acceptable salts and/or solvates thereof.

8. A compound of Formula (I),

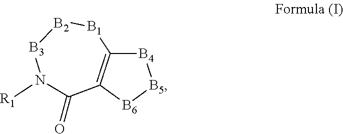

wherein:
B₁ is a carbon centre or nitrogen centre; and either:
(i) B₂ and B₃ together form a single bond between B₁ and the nitrogen atom to which R₁ is attached,
(ii) B₂ is a carbon centre or a nitrogen centre, and B₃ is a single bond between B₂ and the nitrogen atom to which R₁ is attached,
(iii) B₂ is a carbon centre and B₃ is a nitrogen centre, or
(iv) B₂ and B₃ are both carbon centres,
wherein, when B₂ is a carbon centre or nitrogen centre, B₁ is connected to B₂ via either a single or double bond;
wherein, when both B₂ and B₃ are present, B₂ and B₃ are connected via either a single or double bond;
wherein B₁ may, where possible, be optionally substituted with 1 or 2 substituents selected from alkyl, cycloalkyl, alkoxy, OH, CF₃, CN, and halo, or 1 oxo substituent, or 2 substituents which, together with B₁ to which they are both attached, form a 3-, 4-, or 5-membered non-aromatic monocyclic carbon-containing ring, optionally wherein one or two ring members are selected from N, S, and O;
wherein, when B₂ is a carbon centre, B₂ may be optionally substituted with 1 or 2 substituents selected from alkyl, cycloalkyl, alkoxy, OH, CF₃, CN, and halo, or 1 oxo substituent; and
wherein, when B₃ is a carbon centre, B₃ may be optionally substituted with 1 or 2 substituents selected from alkyl, cycloalkyl, alkoxy, OH, CF₃, CN, and halo, or 1 oxo substituent;
and either:
(i) B₄ is N(R₂), B₅ is N, and B₆ is C(R₃), and B₄ is connected to B₅ via a single bond and B₅ is connected to B₆ via a double bond;
(ii) B₄ is C(R₂), B₅ is N, and B₆ is N(R₃), and B₄ is connected to B₅ via a double bond and B₅ is connected to B₆ via a single bond; or
(iii) B₄ is C(R₂), B₅ is C(H), and B₆ is N(R₃), and B₄ is connected to B₅ via a double bond and B₅ is connected to B₆ via a single bond;
R₂ is selected from H, —(CH₂)₀₋₃—C(═O)—N(R₄)(R₅), —C(═O)—O—R₆, —(CH₂)₀₋₃—NR₁₄—C(═O)R₁₅, —(CH₂)₀₋₃—NR₃₄—S(═O)₂R₃₅, alkyl, cycloalkyl, heterocycloalkyl, alkoxy, —OH, —CF₃, halo, aryl, heteroaryl, —CH₂—O—R₁₆ and —CN;
wherein R₄ is H, alkyl, cycloalkyl, and heterocycloalkyl, and
R₅ is selected from alkyl, —(CH₂)₀₋₃-cycloalkyl, —(CH₂)₀₋₃-heterocycloalkyl, —(CH(alkyl))-heterocycloalkyl, —(CH₂)₀₋₃-polycycloalkyl, —(CH₂)₀₋₃-aryl, —(C(H)(phenyl))-alkyl, —(CH₂)₀₋₃heteroaryl^{a}, and —(C(H)(heteroaryl))-alkyl;
or wherein R₄ and R₅ together with the nitrogen atom to which they are attached form a carbon-containing 4-, 5-, 6-, or 7-membered heterocyclic ring which may be saturated or unsaturated with 1 or 2 double bonds, optionally containing one or two additional heteroatoms selected from N, O, and S, wherein the ring may be optionally substituted with 1, 2, or 3 substituents selected from oxo, alkyl, alkoxy, OH, halo and CF_{3;}
R₆ is selected from H, alkyl, —(CH₂)₀₋₃-cycloalkyl, —(CH₂)₀₋₃-heterocycloalkyl, —(CH(alkyl))-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, and —(C(H)(heteroaryl))-alkyl;
R₁₄ is selected from H, alkyl, cycloalkyl, and heterocycloalkyl;
R₁₅ is selected from H, alkyl, —(CH₂)₀₋₃-cycloalkyl, —(CH₂)₀₋₃-heterocycloalkyl, —(CH(alkyl))-heterocycloalkyl, —(CH₂)₀₋₃-aryl, —(C(H)(phenyl))-alkyl, —(CH₂)₀₋₃heteroaryl^{a}, and —(C(H)(heteroaryl))-alkyl;
R₁₆ is selected from H, alkyl, cycloalkyl, and heterocycloalkyl;
R₃₄ is selected from H, alkyl, cycloalkyl, and heterocycloalkyl;
R₃₅ is selected from H, alkyl, —(CH₂)₀₋₃-cycloalkyl, —(CH₂)₀₋₃-heterocycloalkyl, —(CH(alkyl)-heterocycloalkyl, —(CH₂)₀₋₃-aryl, —(C(H)(phenyl)-alkyl, —(CH₂)₀₋₃heteroaryl^{a}, and —(C(H)(heteroaryl)-alkyl;
R₁ is:
(i) Formula (II), wherein Formula (II) is represented by Formula (IIb),

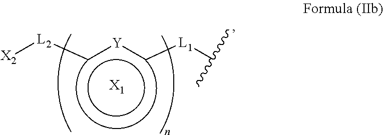

wherein:
n is 1;
L₁ is a linker selected from a single bond, —(CH₂)₁₋₃—, —(CH)(alkyl)-, and —C(alkyl)(alkyl)-;
Y is selected from C, N, O and S;
X₁ is phenyl or a 5- or 6-membered carbon-containing aromatic ring containing one, two or three ring members that are selected from N, S, and O;
wherein X₁ may be optionally substituted with 1 or 2 substituents independently selected from alkyl, cycloalkyl, alkoxy, OH, CF₃, and halo;
L₂ is a linker selected from a single bond, —(CH₂)₁₋₃—, —(CH)(alkyl)-, —C(alkyl)(alkyl)-, —C(═O)—, and —SO₂—; and
X₂ is either:
(a) —NR₂₀R₂₁,
wherein R₂₀ is selected from H, alkyl, and —(CH₂)₀₋₃—R_{22;}
wherein R₂₁ is selected from H, alkyl, and —(CH₂)₀₋₃—R₂₃;
wherein R₂₂ is selected from cycloalkyl, heterocycloalkyl, aryl, heteroaryl,

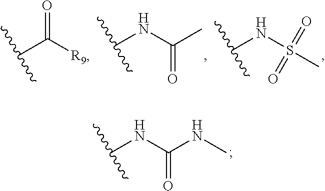

wherein R₂₃ is selected from cycloalkyl, heterocycloalkyl, aryl, heteroaryl,

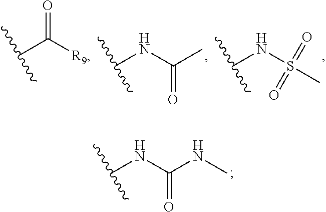

or
(b) 2-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O; or, Formula (III) comprising an aromatic ring A fused to a non-aromatic ring A′,
(ii) Formula (III) comprising an aromatic ring A fused to a non-aromatic ring A′,

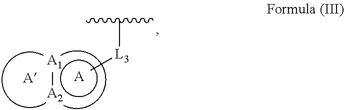

wherein:
L₃ is a linker selected from a single bond, —(CH₂)₁₋₃—, —(CH)(alkyl)-, and —C(alkyl)(alkyl)-;
A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, CF₃, and halo;
A′ is a non-aromatic carbon-containing monocyclic ring containing 5, 6, or 7, ring members, wherein at least one ring member is independently selected from N, S, and O; wherein A′ may be optionally substituted with one R₇, wherein R₇ is selected from alkyl, alkoxy, and —(CH₂)₀₋₃—R₈,
wherein R: is selected from cycloalkyl, heterocycloalkyl, aryl, heteroaryl,

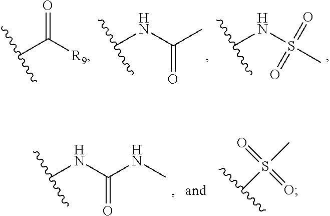

wherein R₉ is selected from H, alkyl, OH, NH₂, and alkoxy;
wherein A′ may be optionally substituted with 1, 2, or 3 further substituents R₁₀, R₁₁, and
R₁₇, wherein R₁₀, R₁₁, and R₁₇, are independently selected from alkyl, cycloalkyl, alkoxy, OH, oxo, CF₃, and halo,
or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O; and
R₃ is phenyl, or a 5- or 6-membered carbon-containing aromatic ring containing one, two or three ring members that are selected from N, S and O, wherein R₃ is substituted with one or two substituents independently selected from small alkyl, small alkoxy, cyclopropyl, OH, CF₃, halo, —C(═O)—NH₂, —C(═O)—N(H)(small alkyl), —C(═O)—N(small alkyl)(small alkyl), and CN; wherein R₃ is not

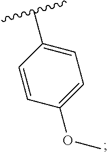 [SMILES: *c1ccc(OC)cc1]

and
alkyl is a linear saturated hydrocarbon having up to 10 carbon atoms (C₁-C₁₀) or a branched saturated hydrocarbon of between 3 and 10 carbon atoms (C₃-C₁₀); alkyl may optionally be substituted with 1, 2 or 3 substituents independently selected from cyclopropyl, (C₁-C₆) alkoxy, OH, —NR₁₂R₁₃, —C(═O) NR₁₂R₁₃, CN, CF₃, and halo;
small alkyl is a linear saturated hydrocarbon having up to 4 carbon atoms (C₁-C₄) or a branched saturated hydrocarbon of between 3 and 4 carbon atoms (C₃-C₄); small alkyl may optionally be substituted with 1 or 2 substituents independently selected from cyclopropyl, OH, CN, CF₃, NH₂, —C(═O)NH₂, and halo;
alkoxy is OCF₃, a linear O-linked hydrocarbon of between 1 and 6 carbon atoms (C₁-C₆), or a branched O-linked hydrocarbon of between 3 and 6 carbon atoms (C₃-C₆); alkoxy may optionally be substituted with 1 or 2 substituents independently selected from cyclopropyl, OH, CN, CF₃, and halo;
small alkoxy is OCF₃, a linear O-linked hydrocarbon of between 1 and 4 carbon atoms (C₁-C₄), or a branched O-linked hydrocarbon of between 3 and 4 carbon atoms (C₃-C₄); small alkoxy may optionally be substituted with 1 or 2 substituents independently selected from cyclopropyl, OH, CN, CF₃, and halo;
aryl is phenyl, biphenyl or naphthyl; aryl may be optionally substituted with 1, 2 or 3 substituents independently selected from alkyl, alkoxy, methylenedioxy, ethylenedioxy, OH, halo, CN, —(CH₂)₀₋₃—NR₁₂R₁₃, OCF₃ and CF_{3;}
polycycloalkyl is a bicyclic saturated hydrocarbon ring system of between 5 and 8 carbon atoms (C₅-C₈), wherein the bicyclic ring is bridged, or is adamantyl; polycycloalkyl may optionally be substituted with 1 or 2 substituents independently selected from alkyl, (C₁-C₅) alkoxy, OH, CN, CF₃, and halo;
cycloalkyl is a monocyclic saturated hydrocarbon ring of between 3 and 6 carbon atoms (C₃-C₆); cycloalkyl may optionally be substituted with 1 or 2 substituents independently selected from alkyl, (C₁-C₆) alkoxy, OH, CN, CF₃, and halo;
halo is F, Cl, Br, or I;
heteroaryl is a 5- or 6-membered carbon-containing aromatic ring containing one, two or three ring members that are selected from N, S, and O; wherein heteroaryl may be optionally substituted with 1, 2 or 3 substituents independently selected from alkyl, alkoxy, cyclopropyl, OH, halo, CN, and CF_{3;}
heteroaryl^{a} is a 5-, 6-, 9- or 10-membered mono- or bi-cyclic aromatic ring containing, where possible, 1, 2, 3 or 4 ring members independently selected from N, S, and O; wherein heteroaryl^{a} may be optionally substituted with 1, 2 or 3 substituents independently selected from alkyl, alkoxy, cyclopropyl, OH, halo, CN, and CF_{3;}
heterocycloalkyl is a non-aromatic carbon-containing mono- or bi-cyclic ring containing 3, 4, 5, 6, or 7 ring members, wherein at least one ring member is independently selected from N, S, and O; heterocycloalkyl may optionally be substituted with 1 or 2 substituents independently selected from alkyl, cyclopropyl, (C₁-C₆) alkoxy, OH, CN, CF₃, oxo, and halo; wherein when heterocycloalkyl is a bi-cyclic ring system, the bicyclic ring system is fused, bridged or spiro;
each R₁₂ and R₁₃ is independently selected from H, alkyl, cycloalkyl, and heterocycloalkyl;
and tautomers, isomers, stereoisomers (including enantiomers, diastereoisomers and racemic and scalemic mixtures thereof), deuterated analogues, and pharmaceutically acceptable salts and/or solvates thereof.

9. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof, wherein B₂ is a carbon centre or a nitrogen centre and B₃ is a single bond between B₂ and the nitrogen atom to which R₁ is attached.

10. A compound according to any of numbered embodiments 1 to 8, or a tautomer, isomer, stereoisomer (including an enantiomer, a diastereoisomer and a racemic and scalemic mixture thereof), a deuterated analogue, and a pharmaceutically acceptable salt and/or solvate thereof, wherein:
(i) B₂ and B₃ together form a single bond between B₁ and the nitrogen atom to which R₁ is attached, or
(ii) B₂ is a carbon centre or a nitrogen centre, and B₃ is a single bond between B₂ and the nitrogen atom to which R₁ is attached.

11. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof, wherein B₂ is a carbon centre and B₃ is a single bond between B₂ and the nitrogen atom to which R₁ is attached.

12. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof, wherein:
(i) B₁ is a carbon centre, and B₂ and B₃ together form a single bond between B₁ and the nitrogen atom to which R₁ is attached,
(ii) B₁ is a carbon centre and B₂ is a carbon centre, and B₁ is connected to B₂ via a single bond, or
(iii) B₁ is a nitrogen centre and B₂ is a carbon centre and B₁ is connected to B₂ via a double bond.

13. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof,
wherein B₁ is a carbon centre and B₂ is a carbon centre, and B₁ is connected to B₂ via a single bond, or
wherein B₁ is a nitrogen centre and B₂ is a carbon centre and B₁ is connected to B₂ via a double bond.

14. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof, wherein B₁ is a carbon centre and B₂ is a carbon centre, and B₁ is connected to B₂ via a single bond.

15. A compound according to any of numbered embodiments 1 to 13, or a tautomer, isomer, stereoisomer (including an enantiomer, a diastereoisomer and a racemic and scalemic mixture thereof), a deuterated analogue, and a pharmaceutically acceptable salt and/or solvate thereof, wherein B₁ is a nitrogen centre.

16. A compound according to numbered embodiment 15, or a tautomer, isomer, stereoisomer (including an enantiomer, a diastereoisomer and a racemic and scalemic mixture thereof), a deuterated analogue, and a pharmaceutically acceptable salt and/or solvate thereof, wherein B₁ is a nitrogen centre and B₂ is a carbon centre and B₁ is connected to B₂ via a double bond.

17. A compound according to numbered embodiment 16, or a tautomer, isomer, stereoisomer (including an enantiomer, a diastereoisomer and a racemic and scalemic mixture thereof), a deuterated analogue, and a pharmaceutically acceptable salt and/or solvate thereof, wherein B₁ is a nitrogen centre and B₂ is a carbon centre and B₁ is connected to B₂ via a double bond, and wherein B₃ is a single bond between B₂ and the nitrogen atom to which R₁ is attached.

18. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof, wherein:
(i) B₄ is N(R₂), B₅ is N, and B₆ is C(R₃), and B₄ is connected to B₅ via a single bond and B₅ is connected to B₆ via a double bond; or
(ii) B₄ is C(R₂), B₅ is N, and B₆ is N(R₃), and wherein B₄ is connected to B₅ via a double bond and B₅ is connected to B₆ via a single bond.

19. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof, wherein B₄ is C(R₂), B₅ is N, and B₆ is N(R₃), and wherein B₄ is connected to B₅ via a double bond and B₅ is connected to B₆ via a single bond.

20. A compound according to any of numbered embodiment 1 to 5, or 7 to 18, or a tautomer, isomer, stereoisomer (including an enantiomer, a diastereoisomer and a racemic and scalemic mixture thereof), a deuterated analogue, and a pharmaceutically acceptable salt and/or solvate thereof, wherein the compound is a compound of formula (Ia) or (Ie):

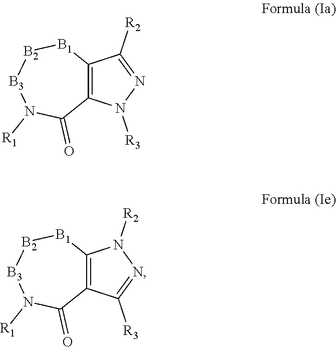

wherein:
B₁ is a carbon centre or nitrogen centre; and either:
B₂ and B₃ together form a single bond between B₁ and the nitrogen atom to which R₁ is attached,
B₂ is a carbon centre and B₃ is a single bond between B₂ and the nitrogen atom to which R₁ is attached, or
B₂ and B₃ are both carbon centres
wherein, when B₂ is a carbon centre, B₁ is connected to B₂ via either a single or double bond; and
wherein, when both B₂ and B₃ are present, B₂ and B₃ are connected via either a single or double bond.

21. A compound according to any of numbered embodiments 18 to 20, or a tautomer, isomer, stereoisomer (including an enantiomer, a diastereoisomer and a racemic and scalemic mixture thereof), a deuterated analogue, and a pharmaceutically acceptable salt and/or solvate thereof, wherein the compound is a compound of formula (Ia):

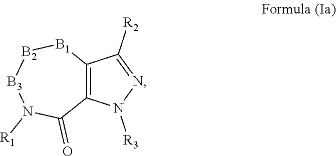

wherein:
B₁ is a carbon centre or nitrogen centre; and either:
(i) B₂ and B₃ together form a single bond between B₁ and the nitrogen atom to which R₁ is attached,
(ii) B₂ is a carbon centre and B₃ is a single bond between B₂ and the nitrogen atom to which R₁ is attached, or
(iii) B₂ and B₃ are both carbon centres
wherein, when B₂ is a carbon centre, B₁ is connected to B₂ via either a single or double bond; and
wherein, when both B₂ and B₃ are present, B₂ and B₃ are connected via either a single or double bond.

22. A compound according to numbered embodiment 20 or numbered embodiment 21, or a tautomer, isomer, stereoisomer (including an enantiomer, a diastereoisomer and a racemic and scalemic mixture thereof), a deuterated analogue, and a pharmaceutically acceptable salt and/or solvate thereof, wherein: B₁ is a carbon centre or nitrogen centre; and either:
(i) B₂ and B₃ together form a single bond between B₁ and the nitrogen atom to which R₁ is attached, or,
(ii) B₂ is a carbon centre and B₃ is a single bond between B₂ and the nitrogen atom to which R₁ is attached
wherein, when B₂ is a carbon centre, B₁ is connected to B₂ via either a single or double bond.

23. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof, wherein B₁ may, where possible, be optionally substituted with 1 or 2 substituents selected from alkyl, cycloalkyl, alkoxy, OH, CF₃, CN, and halo, or 1 oxo substituent.

24. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof, wherein the compound is a compound of formula (Ib), (Ic), (Id), (If), or (Ig):

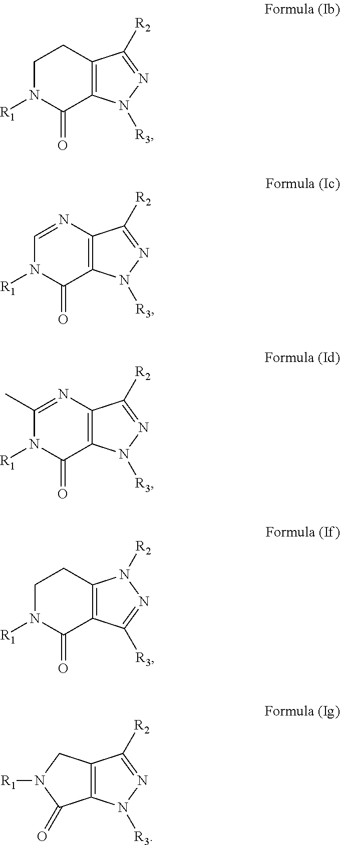

25. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof, wherein the compound is a compound of formula (Ib), (Ic), or (Id):

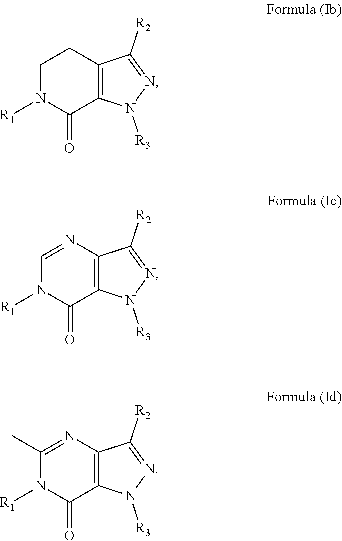

26. A compound according to numbered embodiment 24 or numbered embodiment 25, or a tautomer, isomer, stereoisomer (including an enantiomer, a diastereoisomer and a racemic and scalemic mixture thereof), a deuterated analogue, and a pharmaceutically acceptable salt and/or solvate thereof, wherein, when the compound is a compound of formula (Ic) or (Id), R₂ is —CN.

27. A compound according to numbered embodiment 25 or numbered embodiment 26, or a tautomer, isomer, stereoisomer (including an enantiomer, a diastereoisomer and a racemic and scalemic mixture thereof), a deuterated analogue, and a pharmaceutically acceptable salt and/or solvate thereof, wherein the compound is a compound of formula (Ib) or (Id).

28. A compound according to numbered embodiment 27, or a tautomer, isomer, stereoisomer (including an enantiomer, a diastereoisomer and a racemic and scalemic mixture thereof), a deuterated analogue, and a pharmaceutically acceptable salt and/or solvate thereof, wherein the compound is a compound of formula (Ib).

29. A compound according to numbered embodiment 27, or a tautomer, isomer, stereoisomer (including an enantiomer, a diastereoisomer and a racemic and scalemic mixture thereof), a deuterated analogue, and a pharmaceutically acceptable salt and/or solvate thereof, wherein the compound is a compound of formula (Id).

30. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₂ is selected from —(CH₂)₀₋₃—C(═O)—N(R₄)(R₅), —C(═O)—O—R₆, —(CH₂)₀₋₃—NR₁₄—C(═O)R₁₅, —(CH₂)₀₋₃—NR₃₄—S(═O)₂R₃₅, alkyl, cycloalkyl, heterocycloalkyl, alkoxy, —OH, —CF₃, halo, aryl, heteroaryl, —CH₂—O—R₁₆ and —CN.

31. A compound according to any of numbered embodiments 1 to 29, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₂ is selected from H, —(CH₂)₀₋₃—C(═O)—N(R₄)(R₅), —C(═O)—O—R₆, —(CH₂)₀₋₃—NR₁₄—C(═O)R₁₅, —(CH₂)₀₋₃—NR₃₄—S(═O)₂R₃₅, alkyl, cycloalkyl, alkoxy, —OH, —CF₃, halo, aryl, heteroaryl, —CH₂—O—R₁₆ and —CN.

32. A compound according to any of numbered embodiments 1 to 29, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₂ is selected from H, —(CH₂)₀₋₃—C(═O)—N(R₄)(R₅), —C(═O)—O—R₆, —(CH₂)₀₋₃—NR₁₄—C(═O)R₁₅, —(CH₂)₀₋₃—NR₃₄—S(═O)₂R₃₅, alkyl, cycloalkyl, heterocycloalkyl, alkoxy, —OH, —CF₃, halo, aryl, —CH₂—O—R₁₆ and —CN.

33. A compound according to any of numbered embodiments 1 to 29, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₂ is selected from H, —(CH₂)₀₋₃—C(═O)—N(R₄)(R₅), —(CH₂)₀₋₃—NR₁₄—C(═O)R₁₅, —(CH₂)₀₋₃—NR₃₄—S(═O)₂R₃₅, alkyl, cycloalkyl, heterocycloalkyl, alkoxy, —OH, —CF₃, halo, aryl, heteroaryl, —CH₂—O—R₁₆ and —CN.

34. A compound according to any of numbered embodiments 1 to 29, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₂ is selected from H, —(CH₂)₀₋₃—C(═O)—N(R₄)(R₅), —C(═O)—O—R₆, —(CH₂)₀₋₃—NR₁₄—C(═O)R₁₅, —(CH₂)₀₋₃—NR₃₄—S(═O)₂R₃₅, alkyl, cycloalkyl, heterocycloalkyl, alkoxy, —OH, —CF₃, halo, aryl, heteroaryl, and —CH₂—O—R_{16.}

35. A compound according to any of numbered embodiments 1 to 29, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₂ is selected from H, —(CH₂)₀₋₃—C(═O)—N(R₄)(R₅), —C(═O)—O—R₆, —(CH₂)₀₋₃—NR₁₄—C(═O)R₁₅, —(CH₂)₀₋₃—NR₃₄—S(═O)₂R₃₅, alkyl, cycloalkyl, heterocycloalkyl, alkoxy, —OH, halo, aryl, heteroaryl, —CH₂—O—R₁₆, and —CN.

36. A compound according to any of numbered embodiments 1 to 29, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₂ is selected from —(CH₂)₀₋₃—C(═O)—N(R₄)(R₅), —C(═O)—O—R₆, —(CH₂)₀₋₃—NR₁₄—C(═O)R₁₅, alkyl, cycloalkyl, heterocycloalkyl, alkoxy, —OH, —CF₃, halo, aryl, heteroaryl, —CH₂—O—R₁₆ and —CN.

37. A compound according to any of numbered embodiments 1 to 29, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₂ is selected from H, —(CH₂)₀₋₃—C(═O)—N(R₄)(R₅), —(CH₂)₀₋₃—NR₁₄—C(═O)R₁₅, alkyl, cycloalkyl, heterocycloalkyl, alkoxy, —OH, —CF₃, halo, aryl, heteroaryl, —CH₂—O—R₁₆ and —CN.

38. A compound according to any of numbered embodiments 1 to 29, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₂ is selected from H, —(CH₂)₀₋₃—C(═O)—N(R₄)(R₅), —C(═O)—O—R₆, —(CH₂)₀₋₃—NR₁₄—C(═O)R₁₅, alkyl, cycloalkyl, heterocycloalkyl, alkoxy, —OH, —CF₃, halo, aryl, heteroaryl, and —CH₂—O—R_{16.}

39. A compound according to any of numbered embodiments 1 to 29, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₂ is selected from H, —(CH₂)₀₋₃—C(═O)—N(R₄)(R₅), —C(═O)—O—R₆, —(CH₂)₀₋₃—NR₁₄—C(═O)R₁₅, alkyl, cycloalkyl, heterocycloalkyl, alkoxy, —OH, halo, aryl, heteroaryl, —CH₂—O—R₁₆, and —CN.

40. A compound according to any of numbered embodiments 1 to 29, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₂ is selected from —(CH₂)₀₋₃—C(═O)—N(R₄)(R₅), —(CH₂)₀₋₃—NR₁₄—C(═O)R₁₅, —(CH₂)₀₋₃—NR₃₄—S(═O)₂R₃₅, alkyl, cycloalkyl, heterocycloalkyl, alkoxy, —OH, —CF₃, halo, aryl, heteroaryl, —CH₂—O—R₁₆ and —CN.

41. A compound according to any of numbered embodiments 1 to 29, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₂ is selected from —(CH₂)₀₋₃—C(═O)—N(R₄)(R₅), —C(═O)—O—R₆, —(CH₂)₀₋₃—NR₁₄—C(═O)R₁₅, —(CH₂)₀₋₃—NR₃₄—S(═O)₂R₃₅, alkyl, cycloalkyl, heterocycloalkyl, alkoxy, —OH, —CF₃, halo, aryl, heteroaryl, and —CH₂—O—R_{16.}

42. A compound according to any of numbered embodiments 1 to 29, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₂ is selected from —(CH₂)₀₋₃—C(═O)—N(RA)(R₅), —C(═O)—O—R₆, —(CH₂)₀₋₃—NR₁₄—C(═O)R₁₅, —(CH₂)₀₋₃—NR₃₄—S(═O)₂R₃₅, alkyl, cycloalkyl, heterocycloalkyl, alkoxy, —OH, halo, aryl, heteroaryl, —CH₂—O—R₁₆, and —CN.

43. A compound according to any of numbered embodiments 1 to 29, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₂ is selected from H, —(CH₂)₀₋₃—C(═O)—N(R₄)(R₅), —(CH₂)₀₋₃—NR₁₄—C(═O)R₁₅, —(CH₂)₀₋₃—NR₃₄—S(═O)₂R₃₅, alkyl, cycloalkyl, heterocycloalkyl, alkoxy, —OH, —CF₃, halo, aryl, heteroaryl, and —CH₂—O—R_{16.}

44. A compound according to any of numbered embodiments 1 to 29, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₂ is selected from H, —(CH₂)₀₋₃—C(═O)—N(R₄)(R₅), —(CH₂)₀₋₃—NR₁₄—C(═O)R₁₅, —(CH₂)₀₋₃—NR₃₄—S(═O)₂R₃₅, alkyl, cycloalkyl, heterocycloalkyl, alkoxy, —OH, halo, aryl, heteroaryl, —CH₂—O—R₁₆, and —CN.

45. A compound according to any of numbered embodiments 1 to 29, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₂ is selected from H—(CH₂)₀₋₃—C(═O)—N(R₄)(R₅), —C(═O)—O—R₆, —(CH₂)₀₋₃—NR₁₄—C(═O)R₁₅, —(CH₂)₀₋₃—NR₃₄—S(═O)₂R₃₅, alkyl, cycloalkyl, heterocycloalkyl, alkoxy, —OH, halo, aryl, heteroaryl, and —CH₂—O—R_{16.}

46. A compound according to any of numbered embodiments 1 to 29, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₂ is selected from —(CH₂)₀₋₃—C(═O)—N(R₄)(R₅), —(CH₂)₀₋₃—NR₁₄—C(═O)R₁₅, alkyl, cycloalkyl, heterocycloalkyl, alkoxy, —OH, —CF₃, halo, aryl, heteroaryl, —CH₂—O—R₁₆ and —CN.

47. A compound according to any of numbered embodiments 1 to 29, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₂ is selected from —(CH₂)₀₋₃—C(═O)—N(R₄)(R₅), —C(═O)—O—R₆, —(CH₂)₀₋₃—NR₁₄—C(═O)R₁₅, alkyl, cycloalkyl, heterocycloalkyl, alkoxy, —OH, —CF₃, halo, aryl, heteroaryl, and —CH₂—O—R_{16.}

48. A compound according to any of numbered embodiments 1 to 29, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₂ is selected from —(CH₂)₀₋₃—C(═O)—N(R₄)(R₅), —C(═O)—O—R₆, —(CH₂)₀₋₃—NR₁₄—C(═O)R₁₅, alkyl, cycloalkyl, heterocycloalkyl, alkoxy, —OH, halo, aryl, heteroaryl, —CH₂—O—R₁₆, and —CN.

49. A compound according to any of numbered embodiments 1 to 29, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₂ is selected from H, —(CH₂)₀₋₃—C(═O)—N(R₄)(R₅), —(CH₂)₀₋₃—NR₁₄—C(═O)R₁₅, alkyl, cycloalkyl, heterocycloalkyl, alkoxy, —OH, —CF₃, halo, aryl, heteroaryl, and —CH₂—O—R_{16.}

50. A compound according to any of numbered embodiments 1 to 29, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₂ is selected from H, —(CH₂)₀₋₃—C(═O)—N(R₄)(R₅), —(CH₂)₀₋₃—NR₁₄—C(═O)R₁₅, alkyl, cycloalkyl, heterocycloalkyl, alkoxy, —OH, halo, aryl, heteroaryl, —CH₂—O—R₁₆, and —CN.

51. A compound according to any of numbered embodiments 1 to 29, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₂ is selected from H, —(CH₂)₀₋₃—C(═O)—N(R₄)(R₅), —C(═O)—O—R₆, —(CH₂)₀₋₃—NR₁₄—C(═O)R₁₅, alkyl, cycloalkyl, heterocycloalkyl, alkoxy, —OH, halo, aryl, heteroaryl, and —CH₂—O—R_{16.}

52. A compound according to any of numbered embodiments 1 to 29, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₂ is selected from —(CH₂)₀₋₃—C(═O)—N(R₄)(R₅), —(CH₂)₀₋₃—NR₁₄—C(═O)R₁₅, —(CH₂)₀₋₃—NR₃₄—S(═O)₂R₃₅, alkyl, cycloalkyl, heterocycloalkyl, alkoxy, —OH, —CF₃, halo, aryl, heteroaryl, and —CH₂—O—R_{16.}

53. A compound according to any of numbered embodiments 1 to 29, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₂ is selected from —(CH₂)₀₋₃—C(═O)—N(R₄)(R₅), —(CH₂)₀₋₃—NR₁₄—C(═O)R₁₅, —(CH₂)₀₋₃—NR₃₄—S(═O)₂R₃₅, alkyl, cycloalkyl, heterocycloalkyl, alkoxy, —OH, halo, aryl, heteroaryl, —CH₂—O—R₁₆, and —CN.

54. A compound according to any of numbered embodiments 1 to 29, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₂ is selected from —(CH₂)₀₋₃—C(═O)—N(R₄)(R₅), —C(═O)—O—R₆, —(CH₂)₀₋₃—NR₁₄—C(═O)R₁₅, —(CH₂)₀₋₃—NR₃₄—S(═O)₂R₃₅, alkyl, cycloalkyl, heterocycloalkyl, alkoxy, —OH, halo, aryl, heteroaryl, and —CH₂—O—R_{16.}

55. A compound according to any of numbered embodiments 1 to 29, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₂ is selected from —(CH₂)₀₋₃—C(═O)—N(R₄)(R₅), —(CH₂)₀₋₃—NR₁₄—C(═O)R₁₅, alkyl, cycloalkyl, heterocycloalkyl, alkoxy, —OH, —CF₃, halo, aryl, heteroaryl, and —CH₂—O—R_{16.}

56. A compound according to any of numbered embodiments 1 to 29, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₂ is selected from —(CH₂)₀₋₃—C(═O)—N(R₄)(R₅), —(CH₂)₀₋₃—NR₁₄—C(═O)R₁₅, alkyl, cycloalkyl, heterocycloalkyl, alkoxy, —OH, halo, aryl, heteroaryl, —CH₂—O—R₁₆, and —CN. 57. A compound according to any of numbered embodiments 1 to 29, or a tautomer, isomer, wherein R₂ is selected from —(CH₂)₀₋₃—C(═O)—N(R₄)(R₅), —C(═O)—O—R₆, —(CH₂)₀₋₃—NR₁₄—C(═O)R₁₅, alkyl, cycloalkyl, heterocycloalkyl, alkoxy, —OH, halo, aryl, heteroaryl, and —CH₂—O—R_{16.}

58. A compound according to any of numbered embodiments 1 to 29, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₂ is selected from H, —(CH₂) o-3—C(═O)—N(R₄)(R₅), —(CH₂)₀₋₃—NR₁₄—C(═O)R₁₅, —(CH₂)₀₋₃—NR₃₄—S(═O)₂R₃₅, alkyl, cycloalkyl, heterocycloalkyl, alkoxy, —OH, halo, aryl, heteroaryl, and —CH₂—O—R_{16.}

59. A compound according to any of numbered embodiments 1 to 29, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₂ is selected from H, —(CH₂)₀₋₃—C(═O)—N(R₄)(R₅), —(CH₂)₀₋₃—NR₁₄—C(═O)R₁₅, alkyl, cycloalkyl, heterocycloalkyl, alkoxy, —OH, halo, aryl, heteroaryl, and —CH₂—O—R_{16.}

60. A compound according to any of numbered embodiments 1 to 29, or a tautomer, isomer, stereoisomer (including an enantiomer, a diastereoisomer and a racemic and scalemic mixture thereof), a deuterated analogue, and a pharmaceutically acceptable salt and/or solvate thereof, wherein is R₂ selected from —(CH₂)₀₋₃—C(═O)—N(R₄)(R₅), —(CH₂)₀₋₃—NR₁₄—C(═O)R₁₅, —(CH₂)₀₋₃—NR₃₄—S(═O)₂R₃₅, alkyl, cycloalkyl, heterocycloalkyl, alkoxy, —OH, halo, aryl, heteroaryl, and —CH₂—O—R_{16.}

61. A compound according to any of numbered embodiments 1 to 29, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₂ is selected from —(CH₂)₀₋₃—C(═O)—N(R₄)(R₅), —(CH₂)₀₋₃—NR₁₄—C(═O)R₁₅, alkyl, cycloalkyl, heterocycloalkyl, alkoxy, —OH, halo, aryl, heteroaryl, and —CH₂—O—R_{16.}

62. A compound according to any of numbered embodiments 1 to 29, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₂ is selected from H, —(CH₂)₀₋₃—C(═O)—N(R₄)(R₅), —C(═O)—O—R₆, —CH₂—NH—C(═O)CH₃, —CH₂—NH—S(═O)₂R₃₅, —CH₂—O—R₁₆, —CH₂—OH, —CN, and

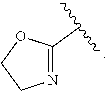 [SMILES: *C1=NCCO1]

63. A compound according to any of numbered embodiments 1 to 29, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₂ from is selected-(CH₂)₀₋₃—C(═O)—N(R₄)(R₅), —C(═O)—O—R₆, —CH₂—NH—C(═O)CH₃, —CH₂—NH—S(═O)₂R₃₅, —CH₂—O—R₁₆, —CH₂—OH, —CN, and

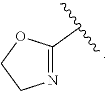 [SMILES: *C1=NCCO1]

64. A compound according to any of numbered embodiments 1 to 29, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₂ is selected from —(CH₂)₀₋₃—C(═O)—N(R₄)(R₅), —C(═O)—O—R₆, —CH₂—NH—C(═O)CH₃, —CH₂—NH—S(═O)₂(cyclopropyl), —CH₂—O—R₁₆, —CH₂—OH, —CN, and

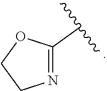 [SMILES: *C1=NCCO1]

65. A compound according to any of numbered embodiments 1 to 29, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₂ is selected from —(CH₂)₀₋₃—C(═O)—N(R₄)(R₅), —C(═O)—O—R₆, —CH₂—NH—S(═O)₂(cyclopropyl), —CH₂—O—CH₃, —CH₂—OH, and —CN.

66. A compound according to any of numbered embodiments 1 to 29, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₂ is selected from H, —(CH₂)₀₋₃—C(═O)—N(R₄)(R₅), —C(═O)—O—R₆, —CH₂—NH—C(═O)CH₃, —CH₂—NH—S(═O)₂R₃₅, —CH₂—OH, —CH₂—O—R₁₆, and

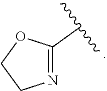 [SMILES: *C1=NCCO1]

67. A compound according to any of numbered embodiments 1 to 29, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₂ is selected from —(CH₂)₀₋₃—C(═O)—N(R₄)(R₅), —C(═O)—O—R₆, —CH₂—NH—C(═O)CH₃, —CH₂—NH—S(═O)₂R₃₅, —CH₂—OH, —CH₂—O—R₁₆, and

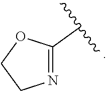 [SMILES: *C1=NCCO1]

68. A compound according to any of numbered embodiments 1 to 29, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₂ is selected from —(CH₂)₀₋₃—C(═O)—N(R₄)(R₅), —C(═O)—O—R₆, —CH₂—NH—C(═O)CH₃, —CH₂—NH—S(═O)₂(cyclopropyl), —CH₂—OH, —CH₂—O—R₁₆, and

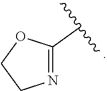 [SMILES: *C1=NCCO1]

69. A compound according to any of numbered embodiments 1 to 29, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₂ is selected from —(CH₂)₀₋₃—C(═O)—N(R₄)(R₅), —C(═O)—O—R₆, —CH₂—NH—S(═O)₂(cyclopropyl), —CH₂—OH, and —CH₂—O—CH₃.

70. A compound according to any of numbered embodiments 1 to 29, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₂ is selected from H, —(CH₂)₀₋₃—C(═O)—N(R₄)(R₅), —CH₂—NH—C(═O)CH₃, —CH₂—NH—S(═O)₂R₃₅, —CH₂—O—R₁₆, —CH₂—OH, —CN, and

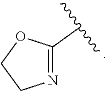 [SMILES: *C1=NCCO1]

71. A compound according to any of numbered embodiments 1 to 29, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₂ is selected from —(CH₂)₀₋₃—C(═O)—N(R₄)(R₅), —CH₂—NH—C(═O)CH₃, —CH₂—NH—S(═O)₂R₃₅, —CH₂—O—R₁₆, —CH₂—OH, —CN, and

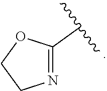 [SMILES: *C1=NCCO1]

72. A compound according to any of numbered embodiments 1 to 29, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₂ is selected from —(CH₂)₀₋₃—C(═O)—N(R₄)(R₅), —CH₂—NH—C(═O)CH₃, —CH₂—NH—S(═O)₂(cyclopropyl), —CH₂—O—R₁₆, —CH₂—OH, —CN, and

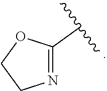 [SMILES: *C1=NCCO1]

73. A compound according to any of numbered embodiments 1 to 29, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₂ is selected from —(CH₂)₀₋₃—C(═O)—N(R₄)(R₅), —CH₂—NH—S(═O)₂(cyclopropyl), —CH₂—O—CH₃, —CH₂—OH, and —CN.

74. A compound according to any of numbered embodiments 1 to 29, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₂ is selected from H, —(CH₂)₀₋₃—C(═O)—N(R₄)(R₅), —CH₂—NH—C(═O)CH₃, —CH₂—NH—S(═O)₂R₃₅, —CH₂—O—R₁₆, —CH₂—OH, and

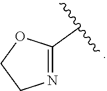 [SMILES: *C1=NCCO1]

75. A compound according to any of numbered embodiments 1 to 29, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₂ is selected from —(CH₂)₀₋₃—C(═O)—N(R₄)(R₅), —CH₂—NH—C(═O)CH₃, —CH₂—NH—S(═O)₂R₃₅, —CH₂—O—R₁₆, —CH₂—OH, and

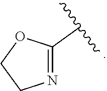 [SMILES: *C1=NCCO1]

76. A compound according to any of numbered embodiments 1 to 29, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₂ is selected from —(CH₂)₀₋₃—C(═O)—N(R₄)(R₅), —CH₂—NH—C(═O)CH₃, —CH₂—NH—S(═O)₂(cyclopropyl), —CH₂—O—R₁₆, —CH₂—OH, and

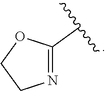 [SMILES: *C1=NCCO1]

77. A compound according to any of numbered embodiments 1 to 29, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₂ is selected from —(CH₂)₀₋₃—C(═O)—N(R₄)(R₅), —CH₂—NH—S(═O)₂(cyclopropyl), —CH₂—O—CH₃, and —CH₂—OH.

78. A compound according to any of numbered embodiments 1 to 29, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₂ is selected from H, —C(═O)—N(R₄)(R₅), —C(═O)—O—R₆, —CH₂—NH—C(═O)CH₃, —CH₂—NH—S(═O)₂R₃₅, —CH₂—O—R₁₆, —CH₂—OH, —CN, and

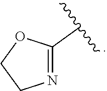 [SMILES: *C1=NCCO1]

79. A compound according to any of numbered embodiments 1 to 29, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₂ is selected from —C(═O)—N(R₄)(R₅), —C(═O)—O—R₆, —CH₂—NH—C(═O)CH₃, —CH₂—NH—S(═O)₂R₃₅, —CH₂—O—R₁₆, —CH₂—OH, —CN, and

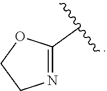 [SMILES: *C1=NCCO1]

80. A compound according to any of numbered embodiments 1 to 29, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₂ is selected from —C(═O)—N(R₄)(R₅), —C(═O)—O—R₆, —CH₂—NH—C(═O)CH₃, —CH₂—NH—S(═O)₂(cyclopropyl), —CH₂—O—R₁₆, —CH₂—OH, —CN, and

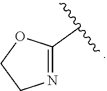 [SMILES: *C1=NCCO1]

81. A compound according to any of numbered embodiments 1 to 29, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₂ is selected from —C(═O)—N(R₄)(R₅), —C(═O)—O—R₆, —CH₂—NH—S(═O)₂(cyclopropyl), —CH₂—O—CH₃, —CH₂—OH, and —CN.

82. A compound according to any of numbered embodiments 1 to 29, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₂ is selected from H, —C(═O)—N(R₄)(R₅), —C(═O)—O—R₆, —CH₂—NH—C(═O)CH₃, —CH₂—NH—S(═O)₂R₃₅, —CH₂—O—R₁₆, —CH₂—OH, and

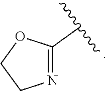 [SMILES: *C1=NCCO1]

83. A compound according to any of numbered embodiments 1 to 29, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₂ is selected from —C(═O)—N(R₄)(R₅), —C(═O)—O—R₆, —CH₂—NH—C(═O)CH₃, —CH₂—NH—S(═O)₂R₃₅, —CH₂—O—R₁₆, —CH₂—OH, and

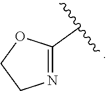 [SMILES: *C1=NCCO1]

84. A compound according to any of numbered embodiments 1 to 29, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₂ is selected from —C(═O)—N(R₄)(R₅), C(═O)—O—R₆, —CH₂—NH—C(═O)CH₃, —CH₂—NH—S(═O)₂(cyclopropyl), —CH₂—O—R₁₆, —CH₂—OH, and

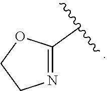 [SMILES: *C1=NCCO1]

85. A compound according to any of numbered embodiments 1 to 29, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₂ is selected from —C(═O)—N(R₄)(R₅), —C(═O)—O—R₆, —CH₂—NH—S(═O)₂(cyclopropyl), —CH₂—O—CH₃, and —CH₂—OH.

86. A compound according to any of numbered embodiments 1 to 29, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₂ is selected from H, —C(═O)—N(R₄)(R₅), —CH₂—NH—C(═O)CH₃, —CH₂—NH—S(═O)₂R₃₅, —CH₂—O—R₁₆, —CH₂—OH, —CN, and

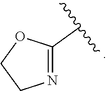 [SMILES: *C1=NCCO1]

87. A compound according to any of numbered embodiments 1 to 29, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₂ is selected from —C(═O)—N(R₄)(R₅), —CH₂—NH—C(═O)CH₃, —CH₂—NH—S(═O)₂R₃₅, —CH₂—O—R₁₆, —CH₂—OH, —CN, and

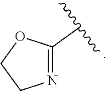 [SMILES: *C1=NCCO1]

88. A compound according to any of numbered embodiments 1 to 29, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₂ is selected from —C(═O)—N(R₄)(R₅), —CH₂—NH—C(═O)CH₃, —CH₂—NH—S(═O)₂(cyclopropyl), —CH₂—O—R₁₆, —CH₂—OH, —CN, and

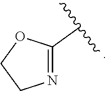 [SMILES: *C1=NCCO1]

89. A compound according to any of numbered embodiments 1 to 29, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₂ is selected from —C(═O)—N(R₄)(R₅), —CH₂—NH—S(═O)₂(cyclopropyl), —CH₂—O—CH₃, —CH₂—OH, and —CN.

90. A compound according to any of numbered embodiments 1 to 29, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₂ is selected from H, —C(═O)—N(R₄)(R₅), —CH₂—NH—C(═O)CH₃, —CH₂—NH—S(═O)₂R₃₅, —CH₂—OH, —CH₂—O—R₁₆, and

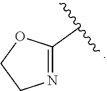 [SMILES: *C1=NCCO1]

91. A compound according to any of numbered embodiments 1 to 29, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₂ is selected from —C(═O)—N(R₄)(R₅), —CH₂—NH—C(═O)CH₃, —CH₂—NH—S(═O)₂R₃₅, —CH₂—OH, —CH₂—O—R₁₆, and

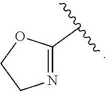 [SMILES: *C1=NCCO1]

92. A compound according to any of numbered embodiments 1 to 29, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₂ is selected from —C(═O)—N(R₄)(R₅), —CH₂—NH—C(═O)CH₃, —CH₂—NH—S(═O)₂(cyclopropyl), —CH₂—OH, —CH₂—O—R₁₆, and

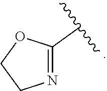 [SMILES: *C1=NCCO1]

93. A compound according to any of numbered embodiments 1 to 29, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₂ is selected from —C(═O)—N(R₄)(R₅), —CH₂—NH—S(═O)₂(cyclopropyl), —CH₂—OH, and —CH₂—O—CH_{3.}

94. A compound according to any of numbered embodiments 1 to 29, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₂ is selected from H, —C(═O)—N(R₄)(R₅), —CH₂—NH—C(═O)CH₃, —CH₂—NH—S(═O)₂R₃₅, —CH₂—OH, and

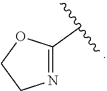 [SMILES: *C1=NCCO1]

95. A compound according to any of numbered embodiments 1 to 29, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₂ is selected from —C(═O)—N(R₄)(R₅), —CH₂—NH—C(═O)CH₃, —CH₂—NH—S(═O)₂R₃₅, —CH₂—OH, and

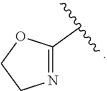 [SMILES: *C1=NCCO1]

96. A compound according to any of numbered embodiments 1 to 29, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₂ is selected from —C(═O)—N(R₄)(R₅), —CH₂—NH—C(═O)CH₃, —CH₂—NH—S(═O)₂(cyclopropyl), —CH₂—OH, and

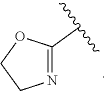 [SMILES: *C1=NCCO1]

97. A compound according to any of numbered embodiments 1 to 29, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₂ is selected from —C(═O)—N(R₄)(R₅), —CH₂—NH—S(═O)₂(cyclopropyl), and —CH₂—OH.

98. A compound according to any of numbered embodiments 1 to 29, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₂ is selected from —(CH₂)₀₋₃—C(═O)—N(R₄)(R₅), —CH₂—NH—S(═O)₂R₃₅, —C(═O)—O—R₆, —CH₂—O—CH₃, and —CN.

99. A compound according to any of numbered embodiments 1 to 29, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₂ is selected from —(CH₂)₀₋₃—C(═O)—N(R₄)(R₅), —C(═O)—O—R₆, —CH₂—O—CH₃, and —CN.

100. A compound according to any of numbered embodiments 1 to 29, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₂ is selected from —(CH₂)₀₋₃—C(═O)—N(R₄)(R₅), —C(═O)—O—R₆, and —CH₂—O—CH_{3.}

101. A compound according to any of numbered embodiments 1 to 29, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₂ is selected from —(CH₂)₀₋₃—C(═O)—N(R₄)(R₅), —CH₂—O—CH₃, and —CN.

102. A compound according to any of numbered embodiments 1 to 29, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₂ is selected from —(CH₂)₀₋₃—C(═O)—N(R₄)(R₅), and —CH₂—O—CH_{3.}

103. A compound according to any of numbered embodiments 1 to 29, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₂ is selected-C(═O)—N(R₄)(R₅), from —CH₂—NH—S(═O)₂R₃₅, —C(═O)—O—R₆, —CH₂—O—CH₃, and —CN.

104. A compound according to any of numbered embodiments 1 to 29, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₂ is selected from —C(═O)—N(R₄)(R₅), —C(═O)—O—R₆, —CH₂—O—CH₃, and CN.

105. A compound according to any of numbered embodiments 1 to 29, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₂ is selected from —C(═O)—N(R₄)(R₅), —C(═O)—O—R₆, and —CH₂—O—CH_{3.}

106. A compound according to any of numbered embodiments 1 to 29, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₂ is selected from —C(═O)—N(R₄)(R₅), —CH₂—O—CH₃, and —CN.

107. A compound according to any of numbered embodiments 1 to 29, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₂ is selected from —C(═O)—N(R₄)(R₅), and —CH₂—O—CH_{3.}

108. A compound according to any of numbered embodiments 1 to 29, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₂ is —C(═O)—N(R₄)(R₅).

109. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof, wherein:
R₄ is H or small alkyl, and
R₅ is selected from alkyl, —OH, —S(═O)₂-(small alkyl), —(CH₂)₁₋₃—(C═O)—OH, —(CH₂)₀₋₃-cycloalkyl^{b}, —(CH₂)₀₋₃-heterocycloalkyl, —(CH(alkyl))-heterocycloalkyl, —(CH₂)₀₋₃-polycycloalkyl, —(CH₂)₀₋₃-aryl, —(C(H)(phenyl))-alkyl, —(CH₂)₀₋₃heteroaryl^{a}, —(C(H)(heteroaryl))-alkyl, —(C(H)(alkyl))-(C═O)—OH, and —(C(H)(heteroaryl))-(C═O)—OR_{28;}
or wherein R₄ and R₅ together with the nitrogen atom to which they are attached form a carbon-containing 4-, 5-, or 6-membered saturated heterocyclic ring, optionally containing an additional heteroatom selected from N, and O, which may be optionally mono- or di-substituted with substituents selected from oxo, alkyl, alkoxy, OH, halo and CF₃; and
R₆, where present, is selected from H, alkyl, —(CH₂)₀₋₃-cycloalkyl, —(CH₂)₀₋₃-heterocycloalkyl, —(CH(alkyl))-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, and —(C(H)(heteroaryl))-alkyl.

110. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof, wherein:
R₄ is H or small alkyl, and
R₅ is selected from alkyl, —OH, —S(═O)₂-(small alkyl), —(CH₂)₁₋₃—(C═O)—OH, —(CH₂)₀₋₃-cycloalkyl^{a}, —(CH₂)₀₋₃-heterocycloalkyl, —(CH(alkyl))-heterocycloalkyl, —(CH₂)₀₋₃-polycycloalkyl, —(CH₂)₀₋₃-aryl, —(C(H)(phenyl))-alkyl, —(CH₂)₀₋₃heteroaryl^{a}, —(C(H)(heteroaryl))-alkyl, —(C(H)(alkyl))-(C═O)—OH, and —(C(H)(heteroaryl))-(C═O)—OR_{28;}
or wherein R₄ and R₅ together with the nitrogen atom to which they are attached form a carbon-containing 4-, 5-, or 6-membered saturated heterocyclic ring, optionally containing an additional heteroatom selected from N, and O, which may be optionally mono- or di-substituted with substituents selected from oxo, alkyl, alkoxy, OH, halo and CF₃; and
R₆, where present, is selected from H, alkyl, —(CH₂)₀₋₃-cycloalkyl, —(CH₂)₀₋₃-heterocycloalkyl, —(CH(alkyl))-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, and —(C(H)(heteroaryl))-alkyl.

111. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof, wherein:
R₄ is H or small alkyl, and
R₅ is selected from alkyl, —(CH₂)₀₋₃-cycloalkyl, —(CH₂)₀₋₃-heterocycloalkyl, —(CH(alkyl))-heterocycloalkyl, —(CH₂)₀₋₃-polycycloalkyl, —(CH₂)₀₋₃-aryl, —(C(H)(phenyl))-alkyl, —(CH₂)₀₋₃heteroaryl^{a}, and —(C(H)(heteroaryl))-alkyl;
or wherein R₄ and R₅ together with the nitrogen atom to which they are attached form a carbon-containing 5-, or 6-membered saturated heterocyclic ring, optionally containing an additional heteroatom selected from N, and O, which may be optionally mono- or di-substituted with substituents selected from oxo, alkyl, alkoxy, OH, halo and CF₃; and
R₆, where present, is selected from H, alkyl, —(CH₂)₀₋₃-cycloalkyl, —(CH₂)₀₋₃-heterocycloalkyl, —(CH(alkyl))-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, and —(C(H)(heteroaryl))-alkyl.

112. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof, wherein:
R₄ is H or small alkyl, and
R₅ is selected from alkyl, —(CH₂)₀₋₃-cycloalkyl, —(CH₂)₀₋₃-heterocycloalkyl, —(CH₂)₀₋₃-polycycloalkyl, —(CH₂)₀₋₃-aryl, —(C(H)(phenyl))-alkyl, —(CH₂)₀₋₃heteroaryl^{a}, and —(C(H)(heteroaryl))-alkyl;
or wherein R₄ and R₅ together with the nitrogen atom to which they are attached form a carbon-containing 5-, or 6-membered saturated heterocyclic ring, optionally containing an additional heteroatom selected from N, and O, which may be optionally mono- or di-substituted with substituents selected from oxo, alkyl, alkoxy, OH, halo and CF₃; and
R₆, where present, is selected from H, alkyl, —(CH₂)₀₋₃-cycloalkyl, —(CH₂)₀₋₃-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, and —(C(H)(heteroaryl))-alkyl.

113. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₄ is H or CH_{3.}

114. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₄ is H.

115. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₅ is selected from alkyl, —OH, —S(═O)₂-(small alkyl), —(CH₂)₁₋₂—(C═O)—OH, —(CH₂)₀₋₂-cycloalkyl^{b}, —(CH₂)₀₋₂-heterocycloalkyl, —(CH(alkyl))-heterocycloalkyl, —(CH₂)₀₋₂-aryl, —(CH₂)₀₋₂-polycycloalkyl, —(C(H)(phenyl))-alkyl, —(CH₂)₀₋₂heteroaryl^{a}, —(C(H)(heteroaryl))-alkyl, —(C(H)(alkyl))-(C═O)—OH, and —(C(H)(heteroaryl))-(C═O)—OR_{28.}

116. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₅ is selected from alkyl, —OH, —S(═O)₂-(small alkyl), —(CH₂)₁₋₂—(C═O)—OH, —(CH₂)₀₋₂-cycloalkyl^{a}, —(CH₂)₀₋₂-heterocycloalkyl, —(CH(alkyl))-heterocycloalkyl, —(CH₂)₀₋₂-aryl, —(CH₂)₀₋₂-polycycloalkyl, —(C(H)(phenyl))-alkyl, —(CH₂)₀₋₂heteroaryl^{a}, —(C(H)(heteroaryl))-alkyl, —(C(H)(alkyl))-(C═O)—OH, and —(C(H)(heteroaryl))-(C═O)—OR_{28.}

117. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₅ is selected from alkyl, —(CH₂)₀₋₂-cycloalkyl, —(CH₂)₀₋₂-heterocycloalkyl, —(CH(alkyl))-heterocycloalkyl, —(CH₂)₀₋₂-aryl, —(CH₂)₀₋₂-polycycloalkyl, —(C(H)(phenyl)-alkyl, —(CH₂)₀₋₂heteroaryl^{a}, and —(C(H)(heteroaryl))-alkyl.

118. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₅ is selected from alkyl, —(CH₂)₀₋₂-cycloalkyl, —(CH₂)₀₋₂-heterocycloalkyl, —(CH₂)₀₋₂-aryl, —(CH₂)₀₋₂-polycycloalkyl, —(C(H)(phenyl))-alkyl, —(CH₂)₀₋₂heteroaryl^{a}, and —(C(H)(heteroaryl))-alkyl.

119. A compound according to any one of numbered embodiments 1 to 115, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₅ is selected from alkyl, —OH, —S(═O)₂-(small alkyl), —(CH₂)₁₋₂—(C═O)—OH, —(CH₂)₀₋₂-cycloalkyl^{b}, —(CH₂)₀₋₂-heterocycloalkyl, —(CH(alkyl))-heterocycloalkyl, —(CH₂)₀₋₂-aryl, —(CH₂)₀₋₂-polycycloalkyl, —(C(H)(phenyl))-alkyl, —(CH₂)₀₋₂heteroaryl, —(C(H)(heteroaryl))-alkyl, —(C(H)(alkyl))-(C═O)—OH, and —(C(H)(heteroaryl))-(C═O)—OR_{28.}

120. A compound according to numbered embodiment 116, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₅ is selected from alkyl, —OH, —S(═O)₂-(small alkyl), —(CH₂)₁₋₂—(C═O)—OH, —(CH₂)₀₋₂-cycloalkyl^{a}, —(CH₂)₀₋₂-heterocycloalkyl, —(CH(alkyl))-heterocycloalkyl, —(CH₂)₀₋₂-aryl, —(CH₂)₀₋₂-polycycloalkyl, —(C(H)(phenyl))-alkyl, —(CH₂)₀₋₂heteroaryl, —(C(H)(heteroaryl))-alkyl, —(C(H)(alkyl))-(C═O)—OH, and —(C(H)(heteroaryl))-(C═O)—OR_{28.}

121. A compound according to numbered embodiment 117, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₅ is selected from alkyl, —(CH₂)₀₋₂-cycloalkyl, —(CH₂)₀₋₂-heterocycloalkyl, —(CH(alkyl))-heterocycloalkyl, —(CH₂)₀₋₂-aryl, —(CH₂)₀₋₂-polycycloalkyl, —(C(H)(phenyl))-alkyl, —(CH₂)₀₋₂heteroaryl, and —(C(H)(heteroaryl))-alkyl.

122. A compound according to numbered embodiment 118, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₅ is selected from alkyl, —(CH₂)₀₋₂-cycloalkyl, —(CH₂)₀₋₂-heterocycloalkyl, —(CH₂)₀₋₂-aryl, —(CH₂)₀₋₂-polycycloalkyl, —(C(H)(phenyl))-alkyl, —(CH₂)₀₋₂heteroaryl, and —(C(H)(heteroaryl))-alkyl.

123. A compound according to numbered embodiment 119, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₅ is selected from alkyl, —OH, —S(═O)₂-(small alkyl), —(CH₂)₁₋₂—(C═O)—OH, —(CH₂)₀₋₂-cycloalkyl^{b}, —(CH₂)₀₋₂-heterocycloalkyl, —(CH(alkyl))-heterocycloalkyl, —(CH₂)₀₋₂-polycycloalkyl, —(CH₂)₀₋₂-phenyl, —(C(H)(phenyl))-alkyl, —(CH₂)₀₋₂heteroaryl, —(C(H)(heteroaryl))-alkyl, —(C(H)(alkyl))-(C═O)—OH, and —(C(H)(heteroaryl))-(C═O)—OR_{28.}

124. A compound according to numbered embodiment 120, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₅ is selected from alkyl, —OH, —S(═O)₂-(small alkyl), —(CH₂)₁₋₂—(C═O)—OH, —(CH₂)₀₋₂-cycloalkyl^{a}, —(CH₂)₀₋₂-heterocycloalkyl, —(CH(alkyl))-heterocycloalkyl, —(CH₂)₀₋₂-polycycloalkyl, —(CH₂)₀₋₂-phenyl, —(C(H)(phenyl))-alkyl, —(CH₂)₀₋₂heteroaryl, —(C(H)(heteroaryl))-alkyl, —(C(H)(alkyl))-(C═O)—OH, and —(C(H)(heteroaryl))-(C═O)—OR_{28.}

125. A compound according to numbered embodiment 121, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₅ is selected from alkyl, —(CH₂)₀₋₂-cycloalkyl, —(CH₂)₀₋₂-heterocycloalkyl, —(CH(alkyl))-heterocycloalkyl, —(CH₂)₀₋₂-polycycloalkyl, —(CH₂)₀₋₂-phenyl, —(C(H)(phenyl))-alkyl, —(CH₂)₀₋₂heteroaryl, and —(C(H)(heteroaryl))-alkyl.

126. A compound according to numbered embodiment 122, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₅ is selected from alkyl, —(CH₂)₀₋₂-cycloalkyl, —(CH₂)₀₋₂-heterocycloalkyl, —(CH₂)₀₋₂-polycycloalkyl, —(CH₂)₀₋₂-phenyl, —(C(H)(phenyl))-alkyl, —(CH₂)₀₋₂heteroaryl, and —(C(H)(heteroaryl))-alkyl.

127. A compound according to numbered embodiment 124, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₅ is selected from —OH, —S(═O)₂-(small alkyl), —(CH₂)₀₋₂-cycloalkyl^{a}, —(CH(alkyl))-heterocycloalkyl, —(CH₂)₁₋₂—(C═O)—OH, —(C(H)(phenyl))-alkyl, —(CH₂)₀₋₂heteroaryl, —(C(H)(alkyl))-(C═O)—OH, and —(C(H)(heteroaryl))-(C═O)—OR_{28.}

128. A compound according to numbered embodiment 125, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₅ is selected from —(CH₂)₀₋₂-cycloalkyl, —(CH(alkyl))-heterocycloalkyl, —(C(H)(phenyl))-alkyl, and —(CH₂)₀₋₂heteroaryl.

129. A compound according to any one of numbered embodiments 1-115, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₅ is selected from alkyl, —(CH₂)₀₋₂-cycloalkyl^{b}, —(CH₂)₀₋₃-heterocycloalkyl, —(CH(alkyl))-heterocycloalkyl, —(CH₂)₀₋₃-polycycloalkyl, —(C(H)(phenyl))-alkyl, and —(CH₂)₀₋₂heteroaryl.

130. A compound according to numbered embodiment 127, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₅ is selected from —(CH₂)₀₋₂-cycloalkyl^{a}, —(C(H)(phenyl))-alkyl, and —(CH₂)₀₋₂heteroaryl.

131. A compound according to numbered embodiment 128, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₅ is selected from —(CH₂)₀₋₂-cycloalkyl, —(C(H)(phenyl))-alkyl, and —(CH₂)₀₋₂heteroaryl.

132. A compound according to numbered embodiment 131, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₅ is-(CH₂)₀₋₂heteroaryl.

133. A compound according to any one of numbered embodiments 1-115, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₅ is selected from alkyl, —OH, —S(═O)₂-(small alkyl), —(CH₂)₁₋₃—(C═O)—OR₃₃, —(CH₂)₁₋₃—(C═O)—N(R₃₆)(R₃₇), —(CH₂)₀₋₃-cycloalkyl^{b}, —(CH₂)₀₋₃-heterocycloalkyl, —(CH(alkyl))-heterocycloalkyl, —(CH₂)₀₋₃-polycycloalkyl, —(C(H)(alkyl))-(C═O)—OR₃₈, and —(C(H)(alkyl))-(C═O)—N(R₃₉)(R₄₀).

134. A compound according to numbered embodiment 133, or a tautomer, isomer, wherein R₅ is selected from alkyl, —(CH₂)₀₋₃-cycloalkyl^{b}, —(CH₂)₀₋₃-heterocycloalkyl, —(CH(alkyl))-heterocycloalkyl, and —(CH₂)₀₋₃-polycycloalkyl.

135. A compound according to numbered embodiment 134, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₅ is selected from alkyl, —(CH₂)₀₋₃-cyclopropyl, —(CH₂)₀₋₃-heterocycloalkyl, —(CH(alkyl))-heterocycloalkyl, and —(CH₂)₀₋₃-polycycloalkyl.

136. A compound according to numbered embodiment 135, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₅ is selected from alkyl, —(CH₂)₀₋₃-cyclopropyl, and —(CH₂)₀₋₃-heterocycloalkyl.

137. A compound according to numbered embodiment 136, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₅ is selected from alkyl and —(CH₂)₀₋₃-heterocycloalkyl.

138. A compound according to numbered embodiment 137, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₅ is selected from alkyl and heterocycloalkyl.

139. A compound according to numbered embodiment 138, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₅ is selected from small alkyl, pyrrolidine, piperidine, and tetrahydrofuran, wherein the pyrrolidine, piperidine, and tetrahydrofuran may optionally be substituted with 1 or 2 substituents independently selected from small alkyl, cyclopropyl, small alkoxy, OH, CN, CF₃, and halo.

140. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₆ is selected from H, small alkyl, and —(CH₂)-phenyl.

141. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₁₄ is selected from H and small alkyl.

142. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₁₅ is selected from H, alkyl, —(CH₂)₀₋₃-cycloalkyl, —(CH₂)₀₋₃-heterocycloalkyl, —(CH(alkyl))-heterocycloalkyl,
—(CH₂)₀₋₃-aryl, —(C(H)(phenyl))-alkyl, —(CH₂)₀₋₃heteroaryl, and —(C(H)(heteroaryl))-alkyl.

143. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₁₅ is selected from H, alkyl, —(CH₂)₀₋₃-cycloalkyl, —(CH₂)₀₋₃-heterocycloalkyl, —(CH₂)₀₋₃-aryl, —(C(H)(phenyl)-alkyl, —(CH₂)₀₋₃heteroaryl, and —(C(H)(heteroaryl))-alkyl.

144. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₁₅ is selected from H and small alkyl.

145. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₁₅ is methyl.

146. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R 16 is selected from H, small alkyl, cycloalkyl, —(CH₂)—(C═O)—OH, heterocycloalkyl, and —(CH₂)-heteroaryl.

147. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₁₆ is selected from H, small alkyl, —(CH₂)—(C═O)—OH, heterocycloalkyl, and —(CH₂)-heteroaryl.

148. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₃₄ is selected from H and small alkyl.

149. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₃₄ is selected from H and CH_{3.}

150. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₃₅ is selected from H, alkyl, —(CH₂)₀₋₂-cycloalkyl, —(CH₂)₀₋₂-heterocycloalkyl, —(CH(alkyl))-heterocycloalkyl, —(CH₂)₀₋₂-polycycloalkyl, —(CH₂)₀₋₂-phenyl, —(C(H)(phenyl))-alkyl, —(CH₂)₀₋₂heteroaryl, and —(C(H)(heteroaryl))-alkyl.

151. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₃₅ is selected from H, alkyl, and cycloalkyl.

152. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₃₅ is selected from H, small alkyl, and cyclopropyl.

153. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₂ is selected from:

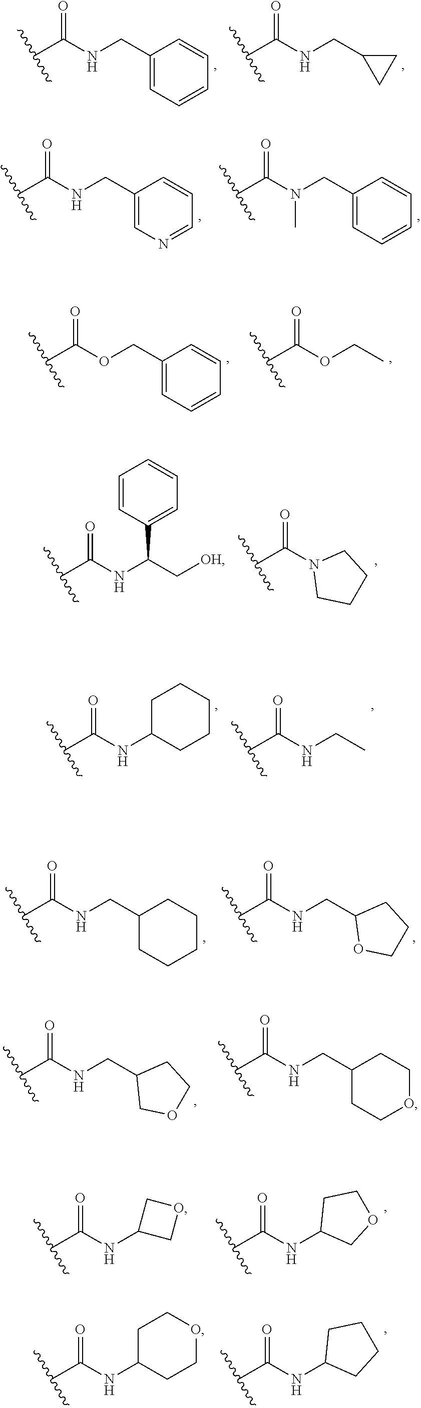

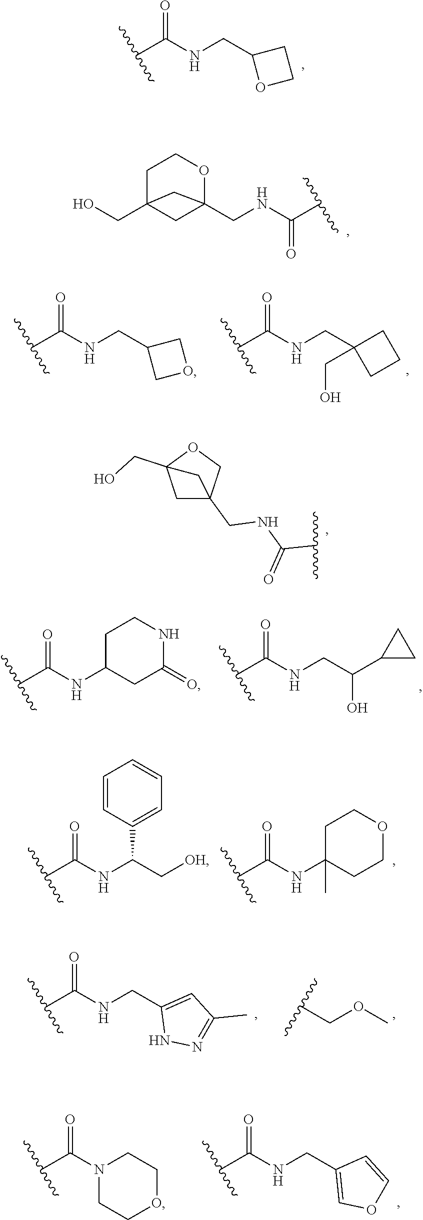

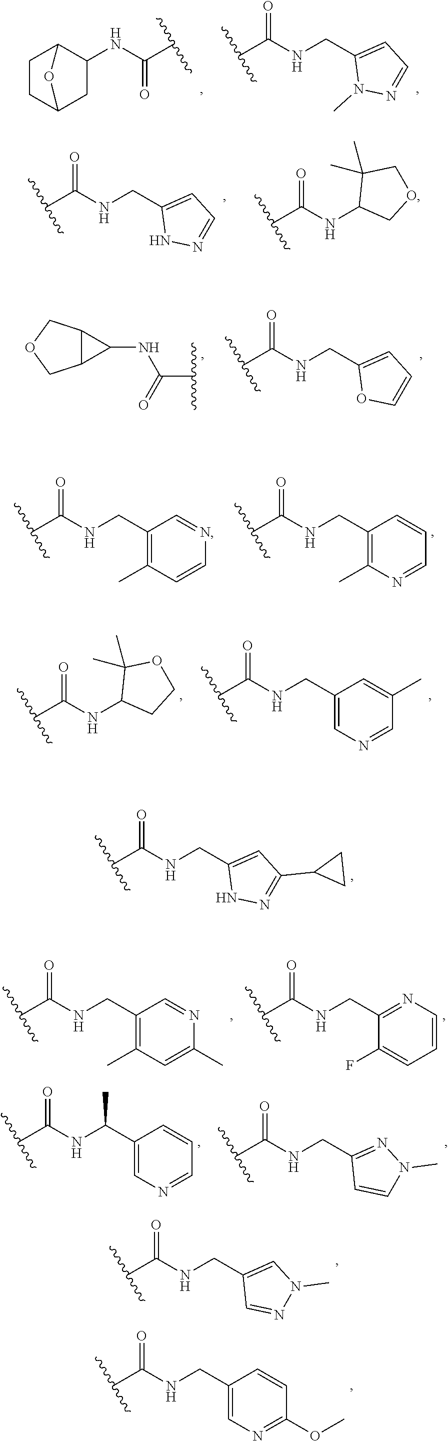

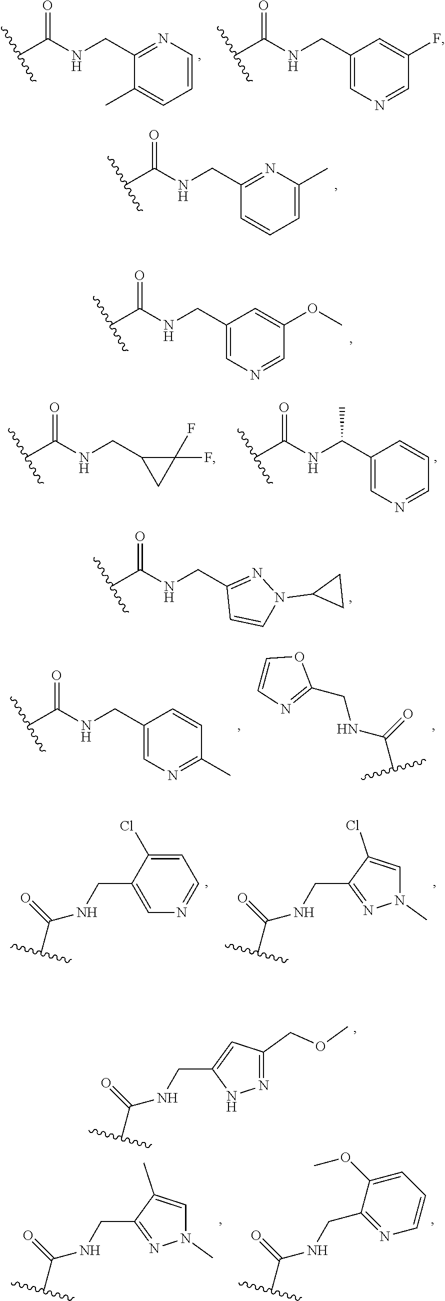

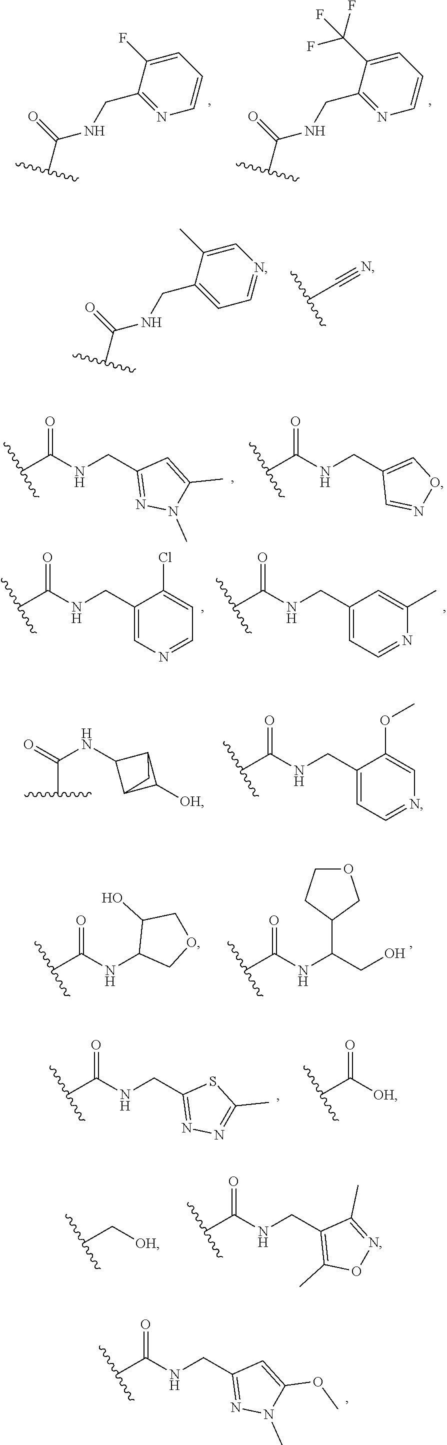

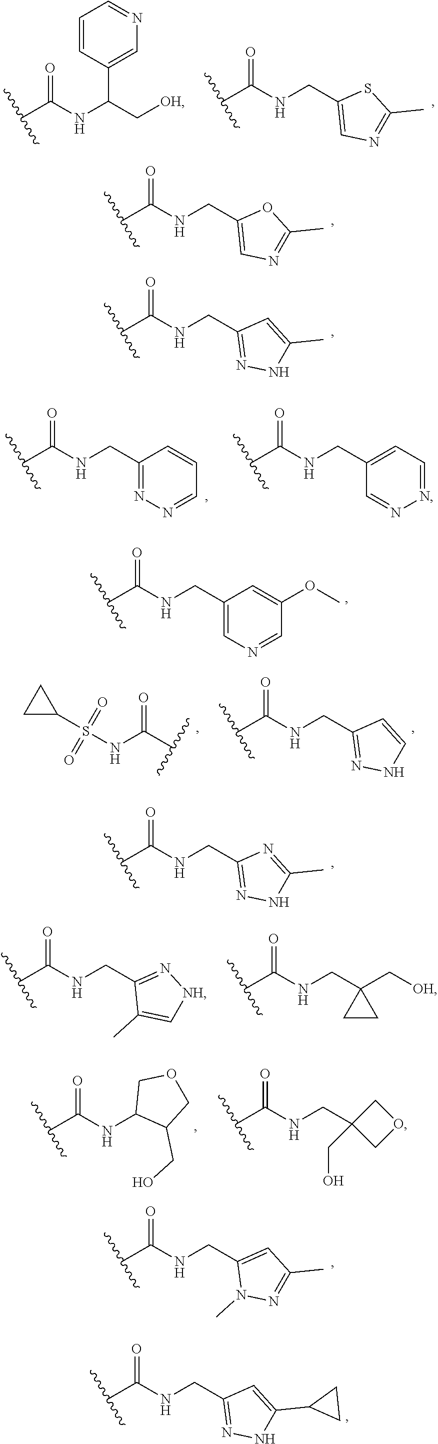

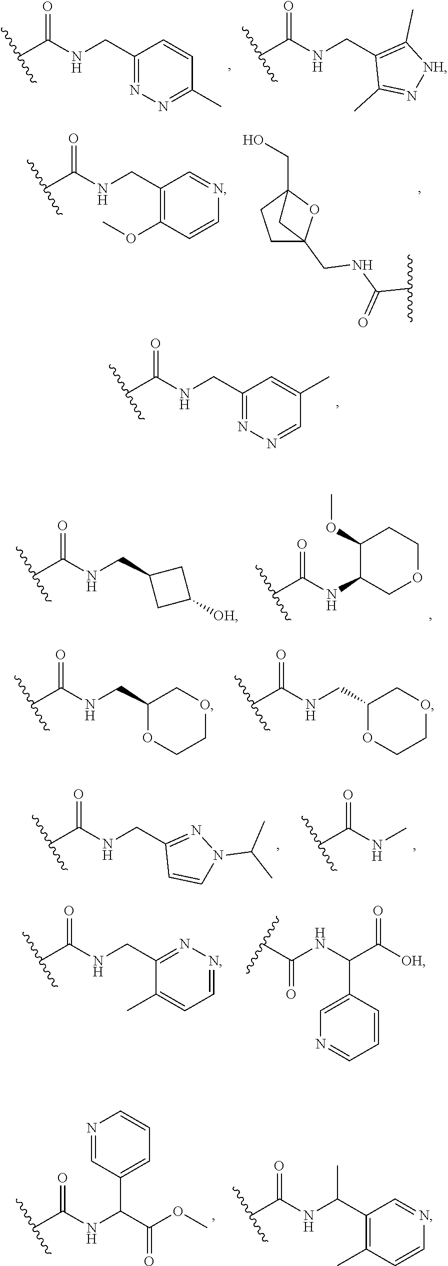

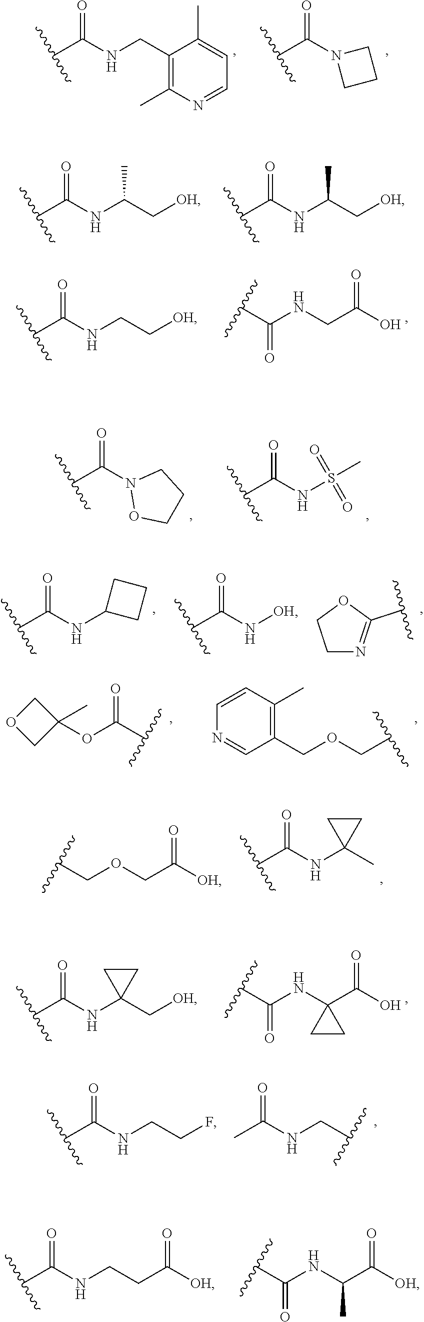

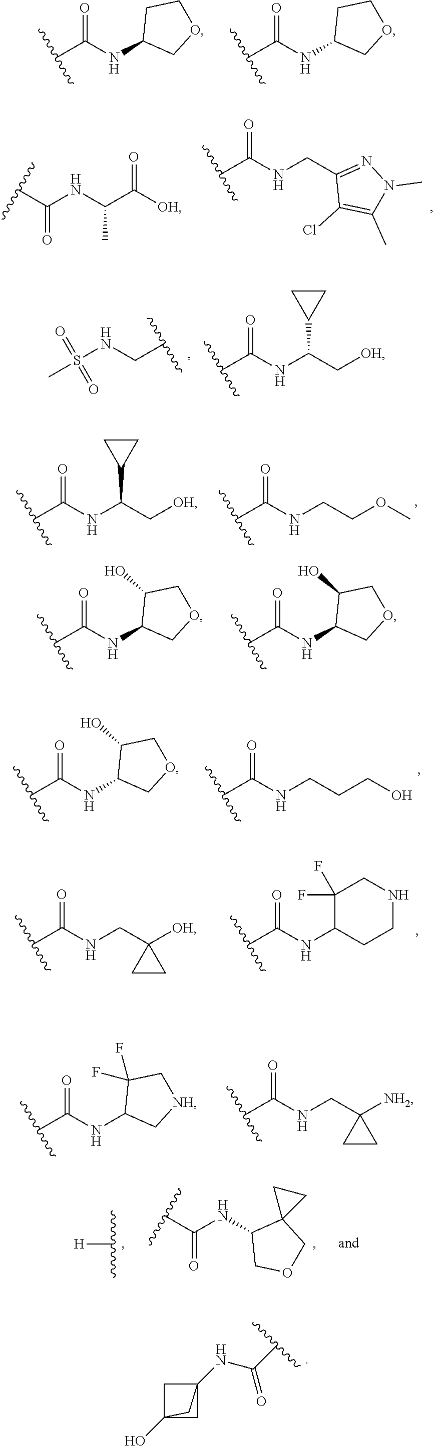

154. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₂ is selected from:

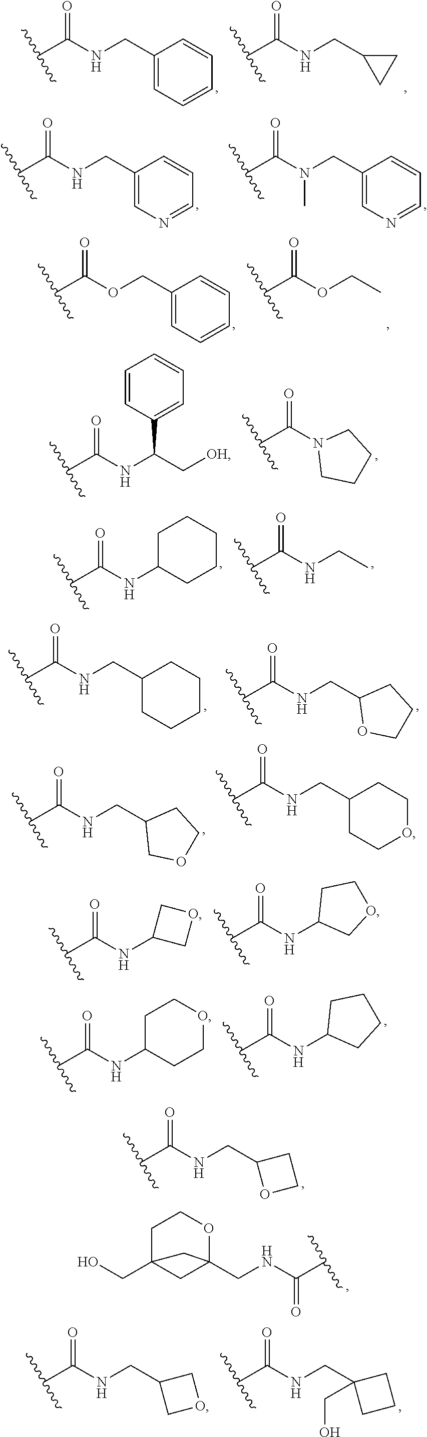

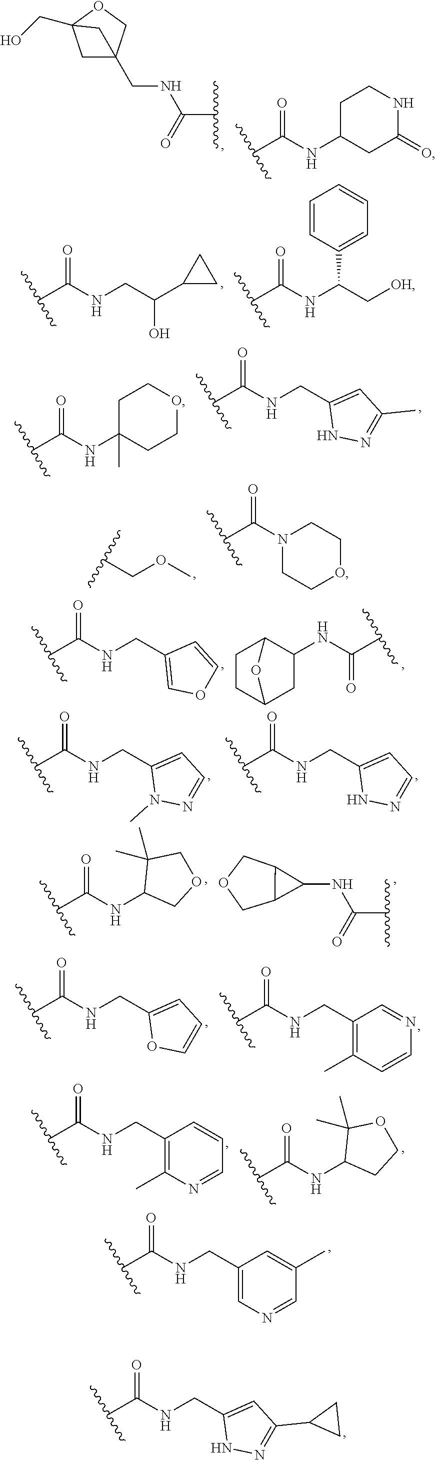

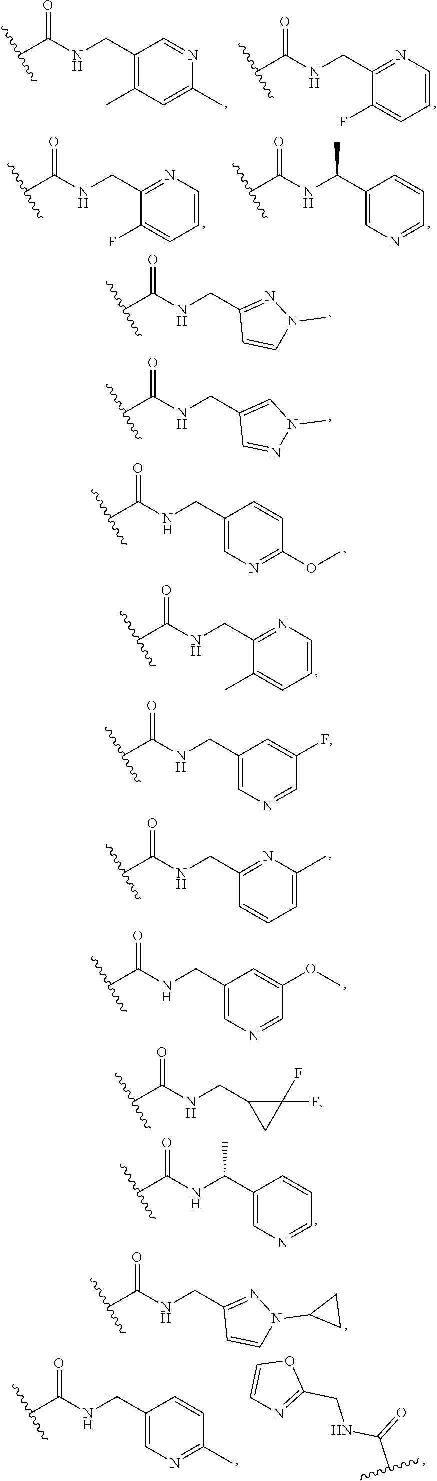

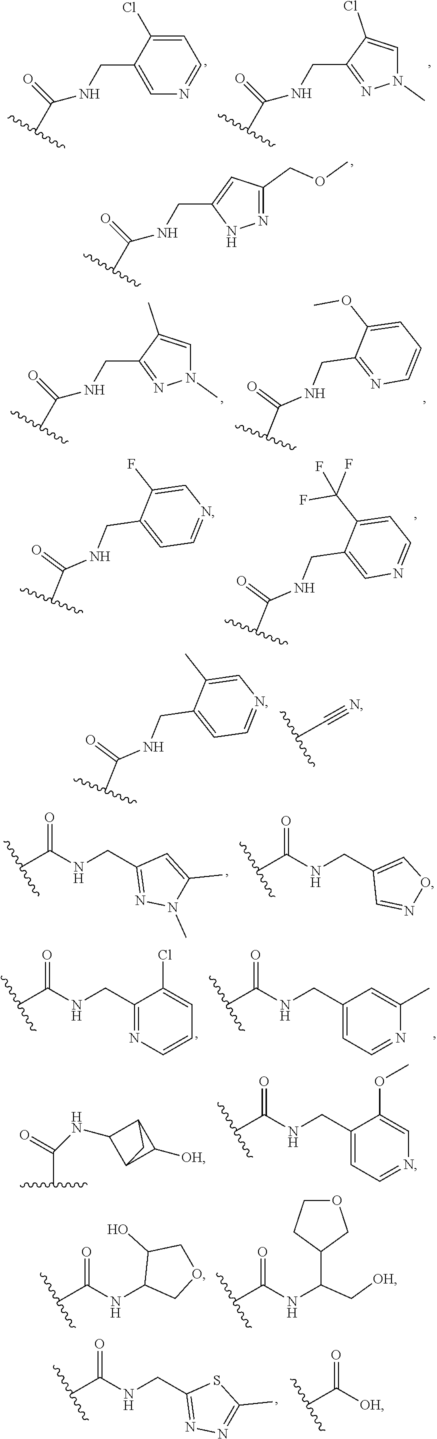

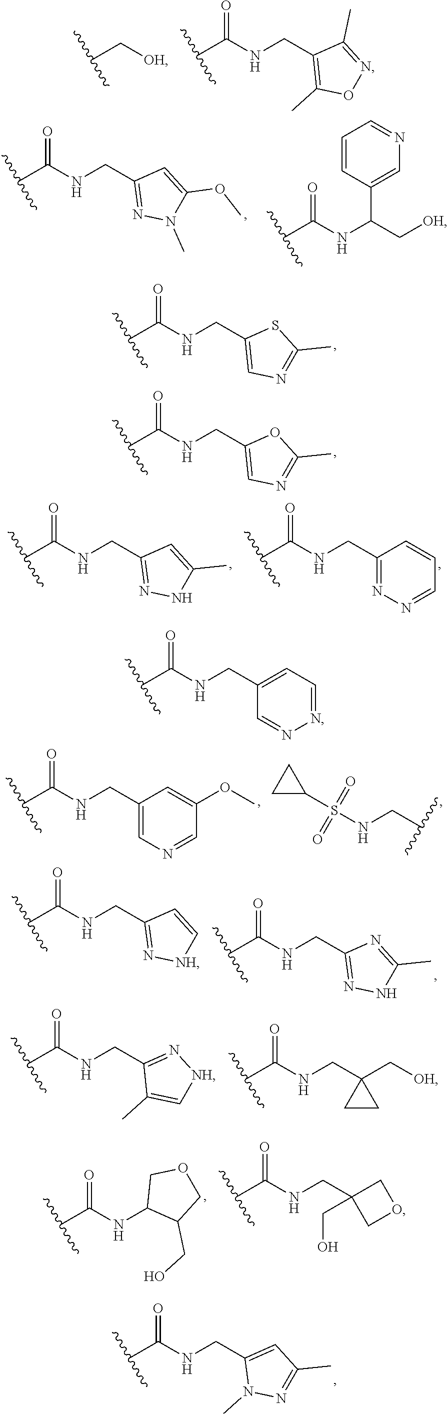

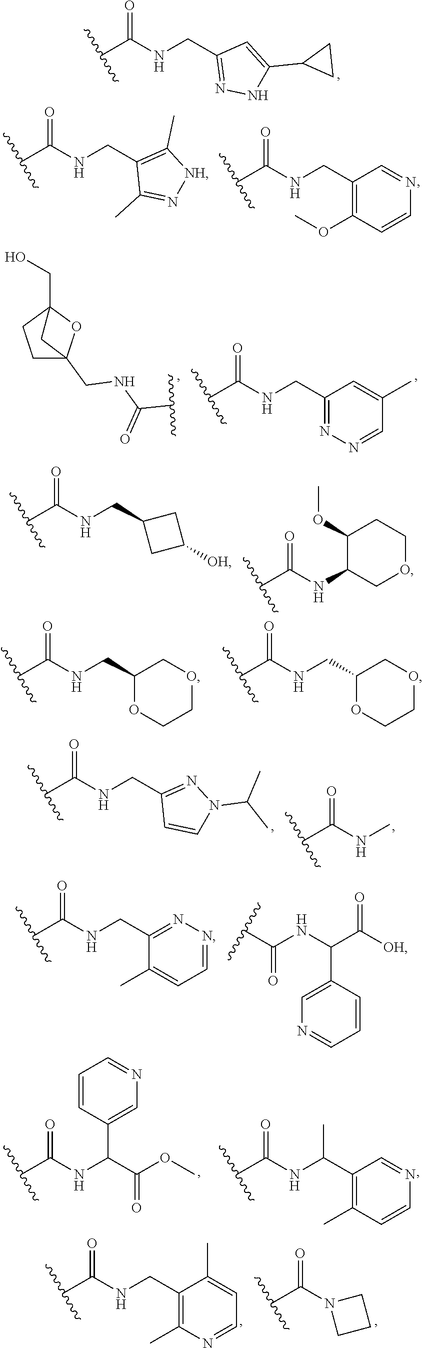

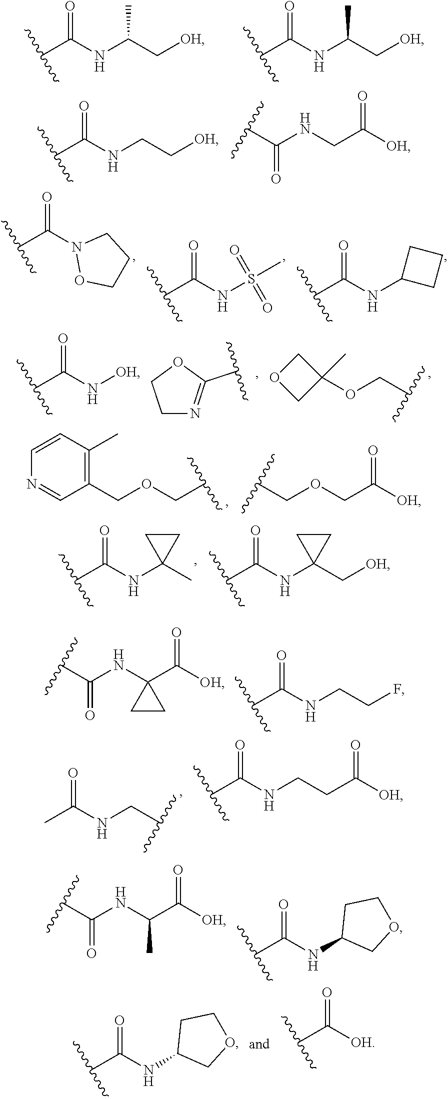

155. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₂ is selected from:

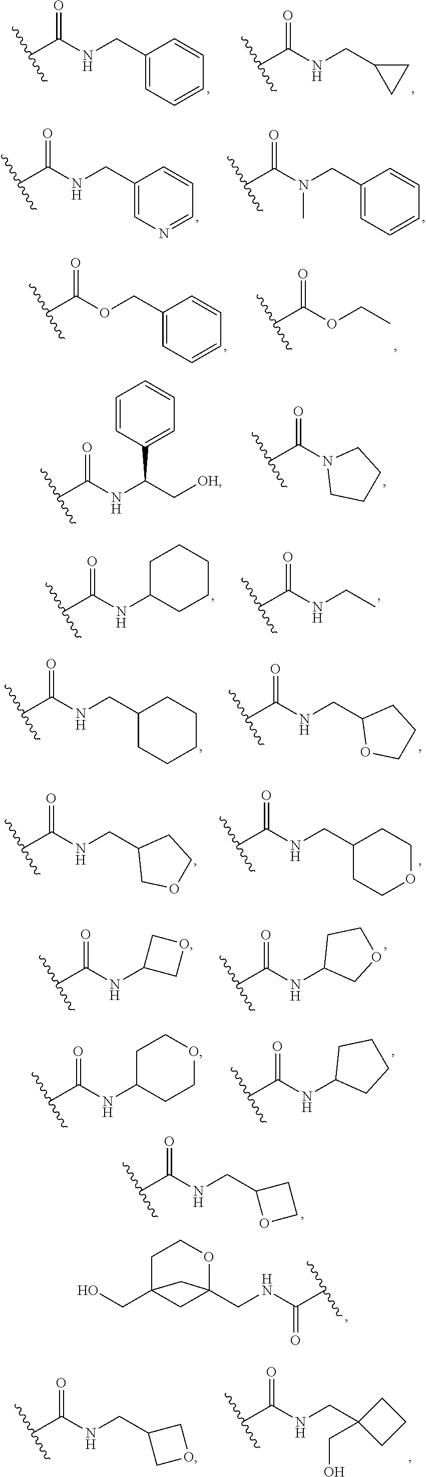

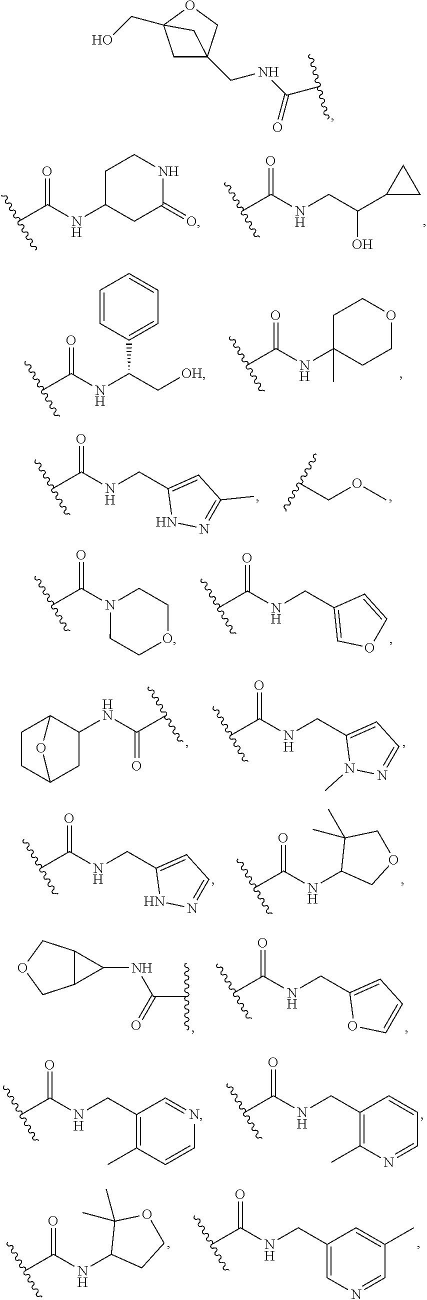

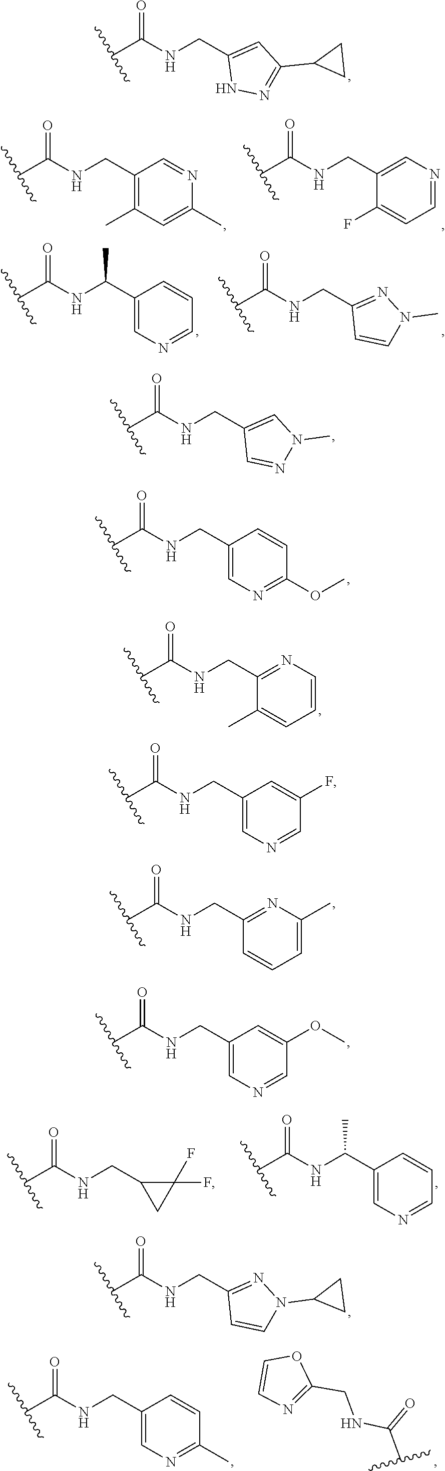

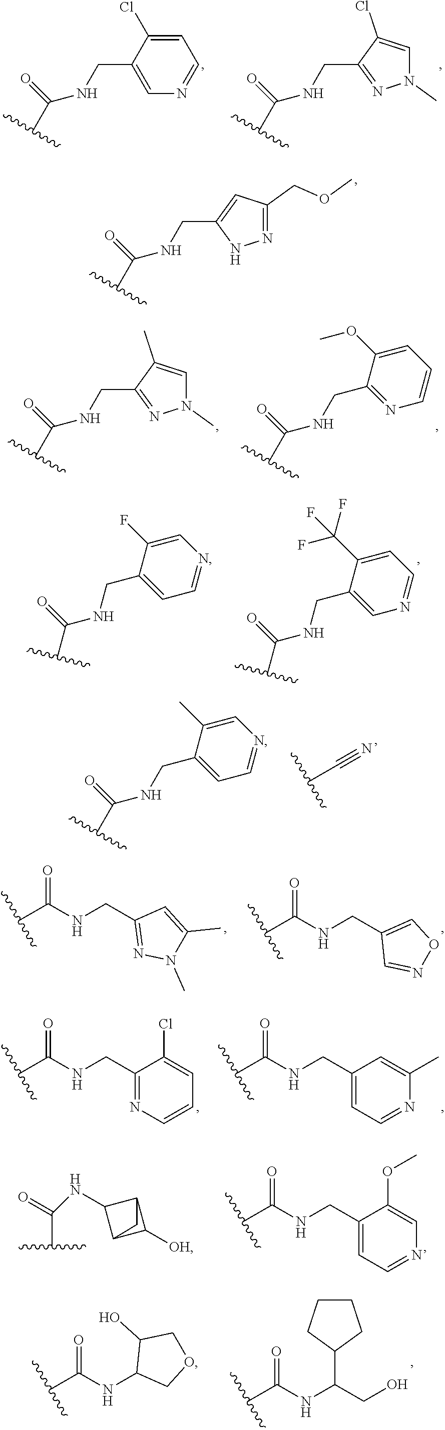

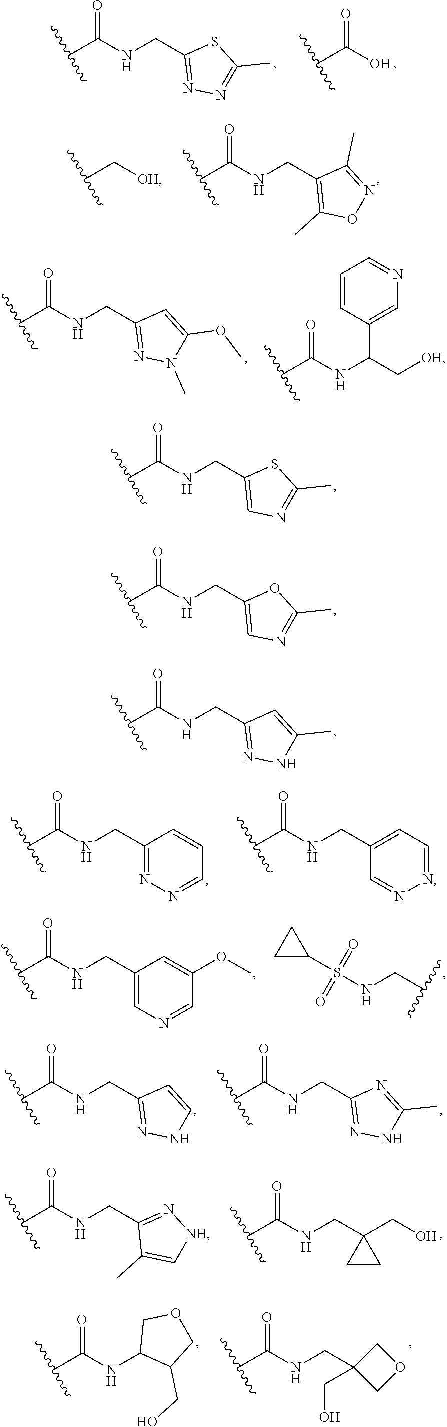

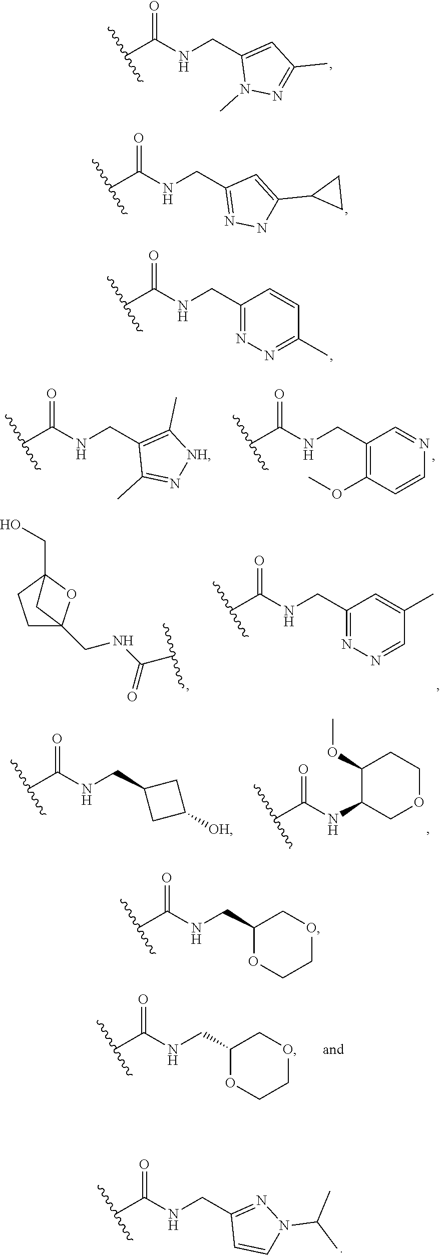

156. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₂ is selected from:

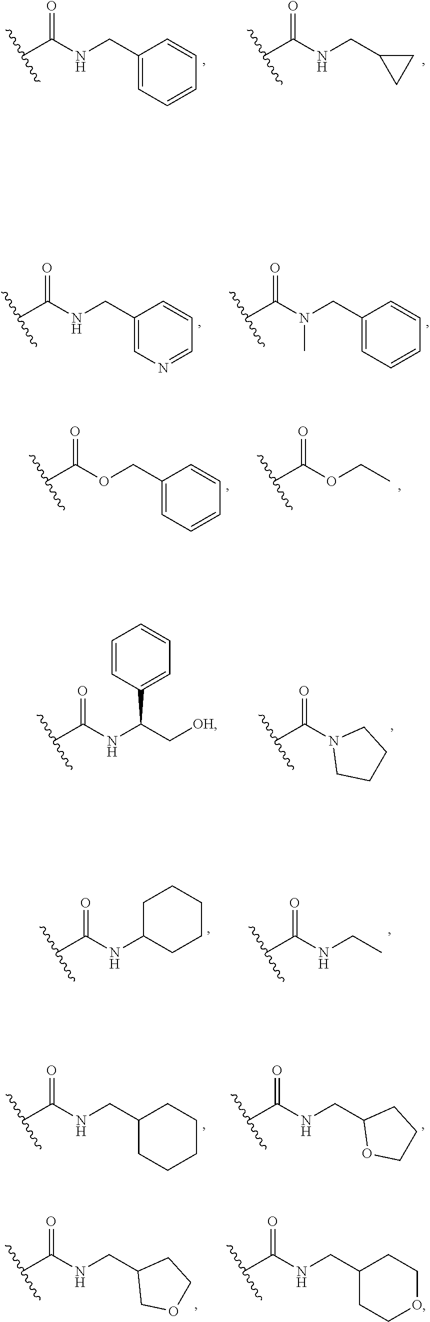

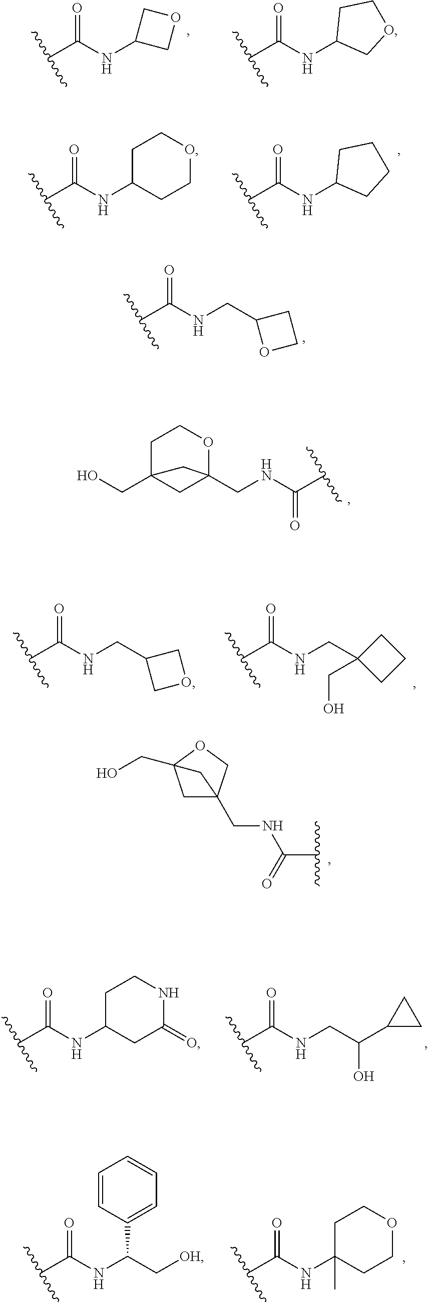

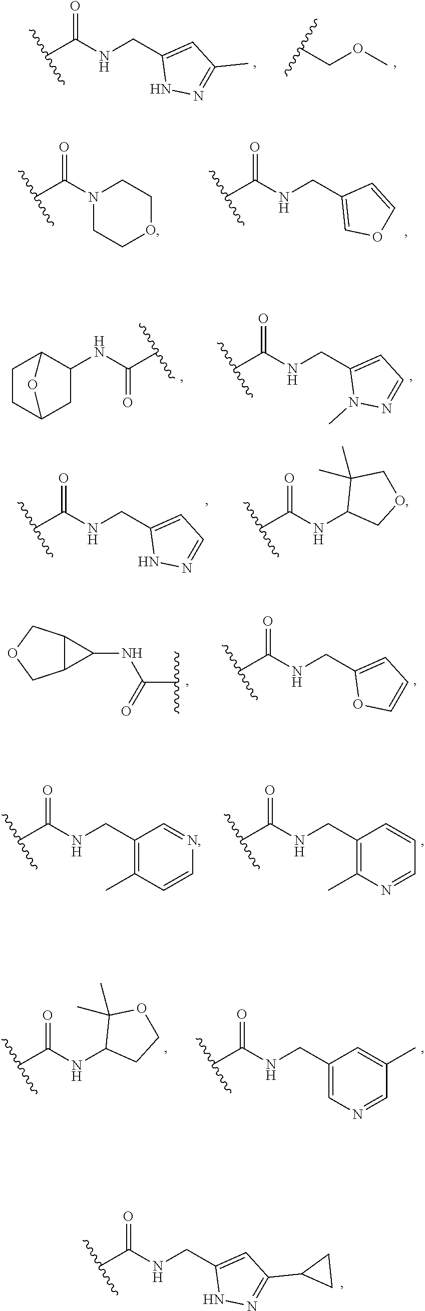

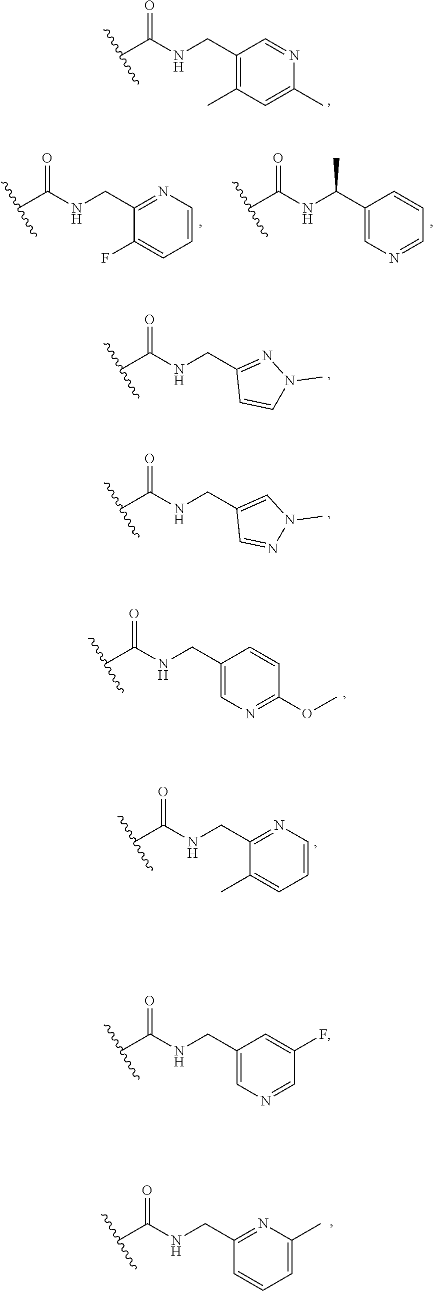

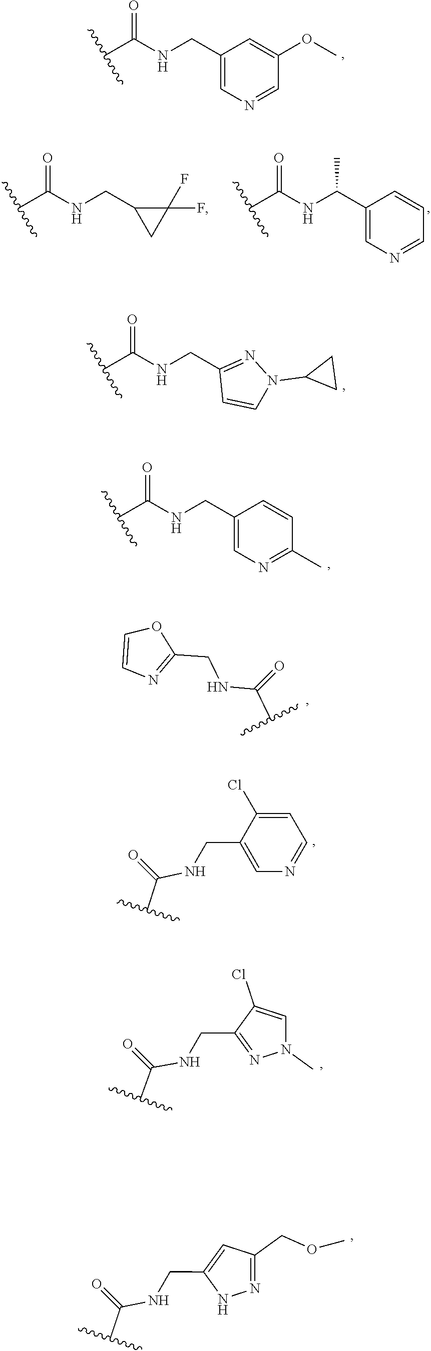

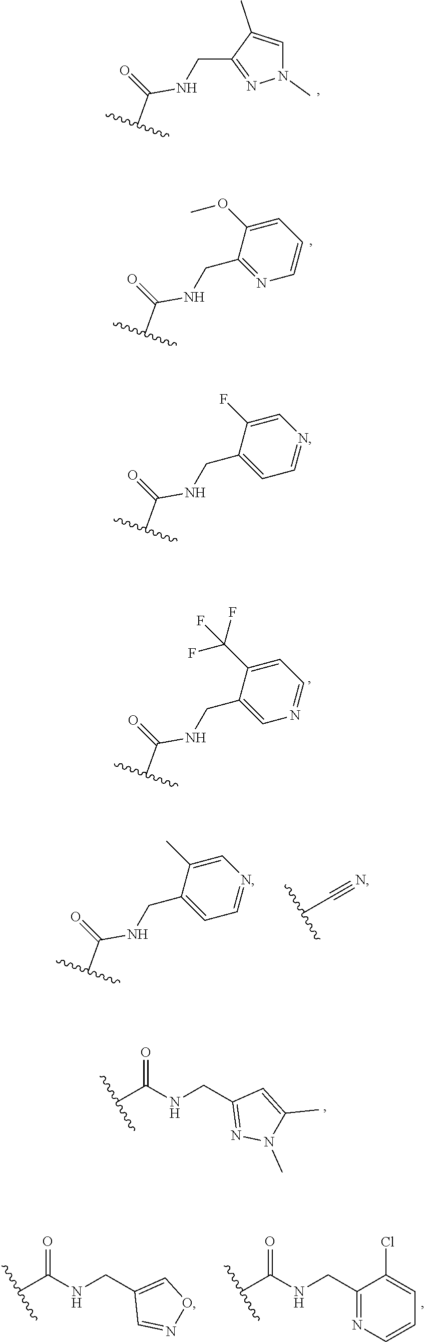

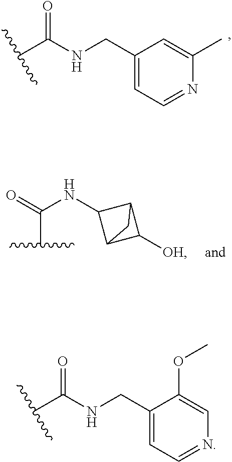 [SMILES: *C(=O)NCc1ccnc(C)c1 ; *C(=O)NC1C2CC1C2O ; *C(=O)NCc1ccncc1OC]

157. A compound according to any of numbered embodiments 1-153, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₂ is selected from:

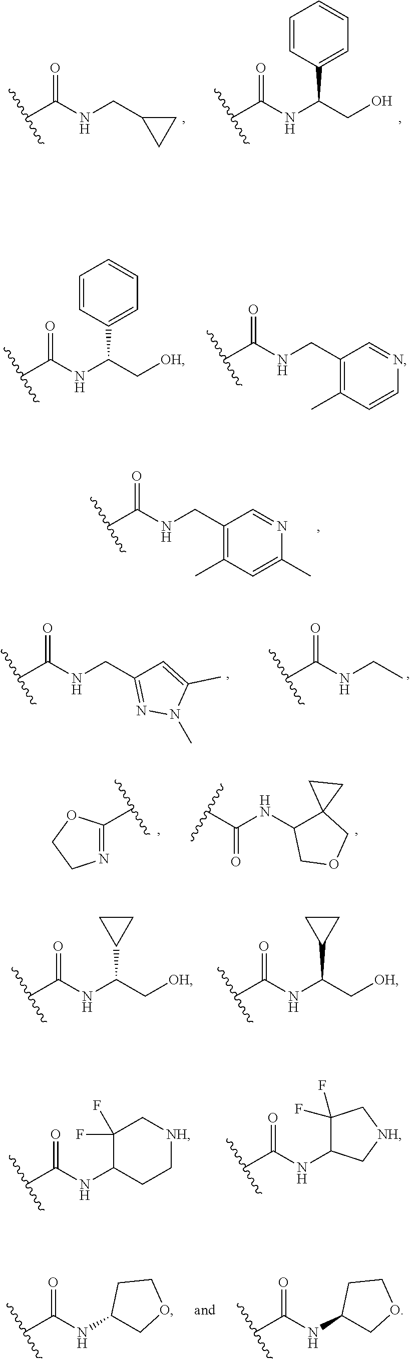

158. A compound according to any of numbered embodiments 1-153, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₂ is selected from:

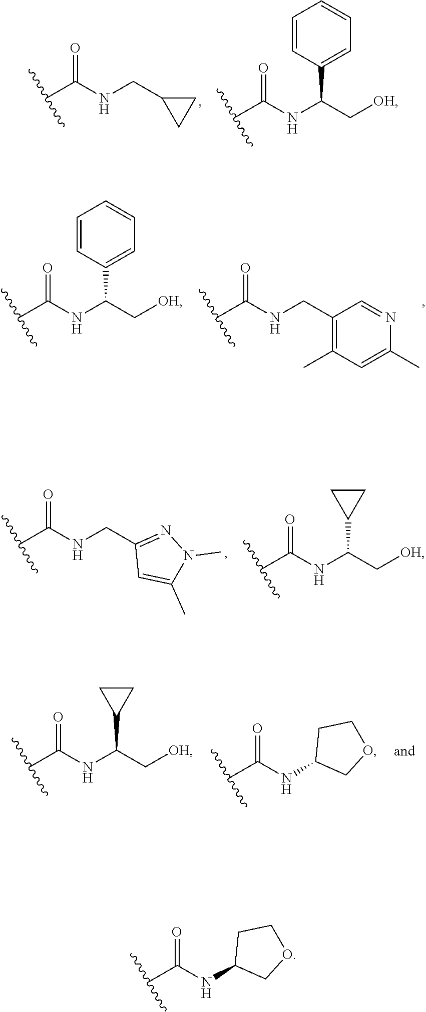

159. A compound according to any of numbered embodiments 1-153, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₂ is selected from:

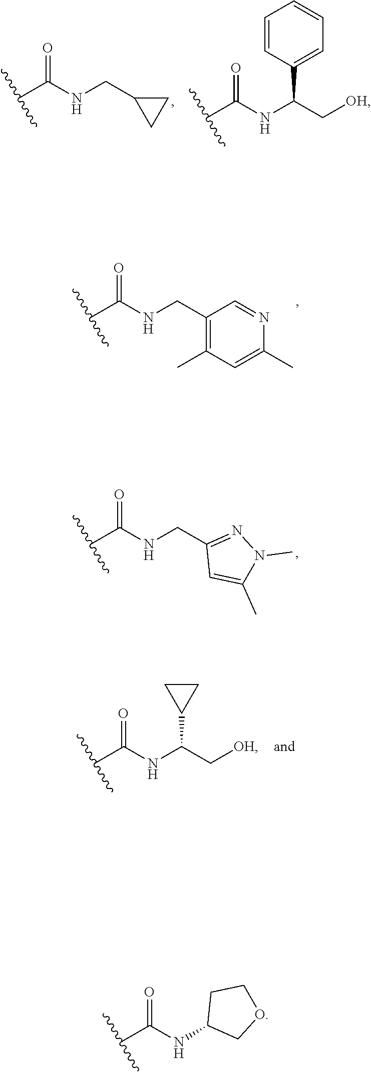

160. A compound according to any of numbered embodiments 1-153, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₂ is selected from:

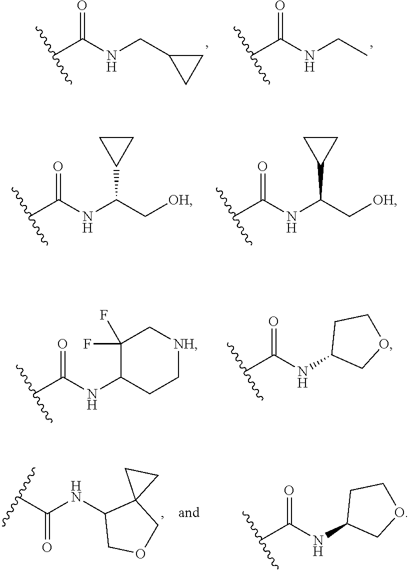

161. A compound according to any of numbered embodiments 1-153, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₂ is selected from:

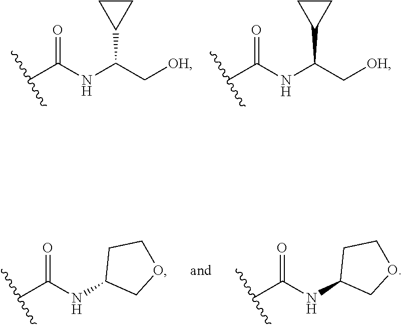 [SMILES: *C(=O)N[C@@H](CO)C1CC1 ; *C(=O)N[C@H](CO)C1CC1 ; *C(=O)N[C@@H]1CCOC1 ; *C(=O)N[C@H]1CCOC1]

162. A compound according to any of numbered embodiments 1-153, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₂ is selected from:

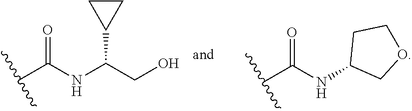 [SMILES: *C(=O)N[C@@H](CO)C1CC1 ; *C(=O)N[C@@H]1CCOC1]

163. A compound according to any of the numbered embodiments 1-154, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₂ is selected from:

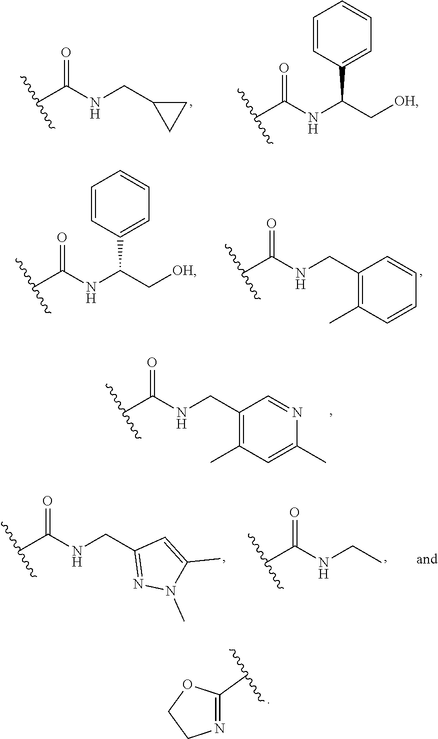

164. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₂ is selected from:

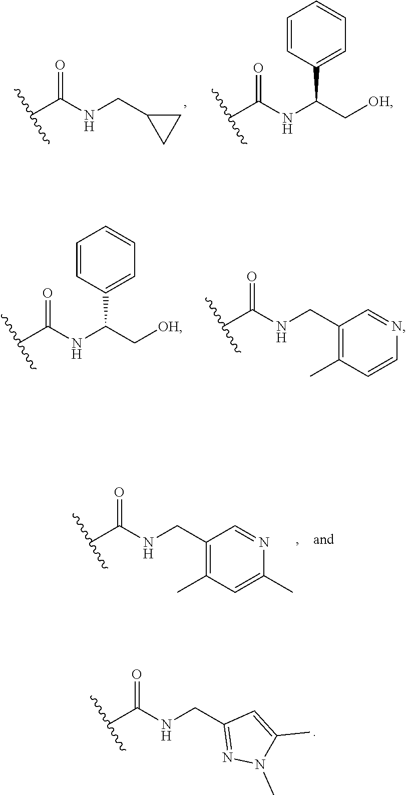 [SMILES: *C(=O)NCC1CC1 ; *C(=O)N[C@H](CO)c1ccccc1 ; *C(=O)N[C@@H](CO)c1ccccc1 ; *C(=O)NCc1cnccc1C ; *C(=O)NCc1cnc(C)cc1C ; *C(=O)NCc1cc(C)n(C)n1]

165. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof, wherein L₁ is a linker selected from a single bond and —(CH₂)₁₋₃ —.

166. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof, wherein L₁ is a single bond.

167. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof, wherein L₂ is a linker selected from a single bond, —(CH₂)₁₋₃—, —(CH)(alkyl)-, —C(alkyl)(alkyl)-, and —SO₂—.

168. A compound according to any of numbered embodiments 1 to 166, or a tautomer, isomer, stereoisomer (including an enantiomer, a diastereoisomer and a racemic and scalemic mixture thereof), a deuterated analogue, and a pharmaceutically acceptable salt and/or solvate thereof, wherein L₂ is a linker selected from a single bond, —(CH₂)₁₋₃—, and —C(═O)—.

169. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof, wherein L₂ is a linker selected from a single bond and —(CH₂)₁₋₃—.

170. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof, wherein L₂ is a single bond.

171. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof, wherein L₁ and L₂ are each independently a linker selected from a single bond and —(CH₂)₁₋₃ —.

172. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof, wherein L₁ and L₂ are both single bonds.

173. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof, wherein X₁ is selected from thiophene, phenyl and pyridyl.

174. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof, wherein X₁ is selected from phenyl and pyridyl.

175. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof, wherein X₁ is phenyl.

176. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof, wherein X₁ may be optionally substituted with 1 or 2 substituents independently selected from small alkyl, cyclopropyl, small alkoxy, OH, CF₃, and halo.

177. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₂₀ is selected from H and small alkyl.

178. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof, wherein Formula (II) is represented by Formula (IIb),

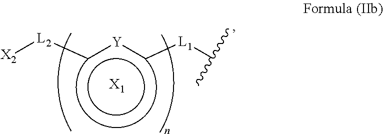

wherein:
n is 0 or 1;
L₁ is a linker selected from a single bond, —(CH₂)₁₋₃—, —(CH)(alkyl)-, and —C(alkyl)(alkyl)-; Y is selected from C, N, O and S;
X₁ is phenyl or 5- or 6-membered aromatic ring containing one, two or three ring members that are selected from N, S, and O; wherein X₁ may be optionally substituted with 1 or 2 substituents independently selected from alkyl, cycloalkyl, alkoxy, OH, O(cyclopropyl), CF₃, and halo;
L₂ is a linker selected from a single bond, —(CH₂)₁₋₃—, —(CH)(alkyl)-, —C(alkyl)(alkyl)-, —C(═O)—, and —SO₂—; and
X₂ is either: (a) —NR₂₀R₂₁,
wherein R₂₀ is selected from H, alkyl, and —(CH₂)₀₋₃—R_{22;}
wherein R₂₁ is selected from H, alkyl, and —(CH₂)₀₋₃—R_{23;}
wherein R₂₂ is selected from cycloalkyl, heterocycloalkyl, aryl, heteroaryl,

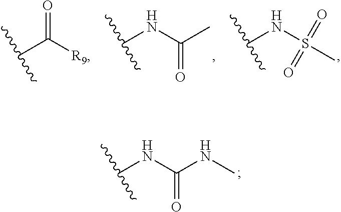

wherein R₂₃ is selected from cycloalkyl, heterocycloalkyl, aryl, heteroaryl,

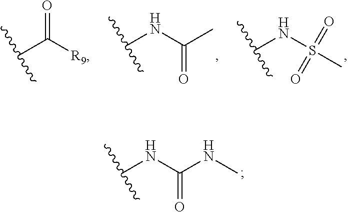

wherein R₉ is selected from H, alkyl, OH, NH₂, and alkoxy; or
(b) 2-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O, and X₂ may be further optionally substituted with 1 or 2 substituents R₂₉ and R₃₀, wherein R₂₉ and R₃₀ are independently selected from alkyl, cycloalkyl, alkoxy, OH, oxo, CF₃, and halo.

179. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof, wherein Formula (II) is represented by Formula (IIb),

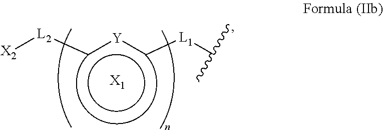

wherein:
n is 0 or 1;
L₁ is a linker selected from a single bond, —(CH₂)₁₋₃—, —(CH)(alkyl)-, and —C(alkyl)(alkyl)-;
Y is selected from C, N, O and S;
X₁ is phenyl or 5- or 6-membered aromatic ring containing one, two or three ring members that are selected from N, S, and O; wherein X₁ may be optionally substituted with 1 or 2 substituents independently selected from alkyl, cycloalkyl, alkoxy, OH, CF₃, and halo;
L₂ is a linker selected from a single bond, —(CH₂)₁₋₃—, —(CH)(alkyl)-, —C(alkyl)(alkyl)-, —C(═O)—, and —SO₂—;and
X₂ is either:
(a) —NR₂₀R₂₁,
wherein R₂₀ is selected from H, alkyl, and —(CH₂)₀₋₃—R_{22;}
wherein R₂₁ is selected from H, alkyl, and —(CH₂)₀₋₃—R₂₃;
wherein R₂₂ is selected from cycloalkyl, heterocycloalkyl, aryl, heteroaryl,

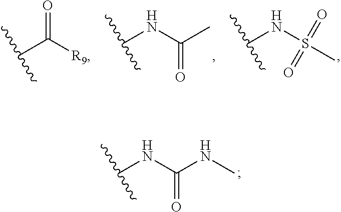

wherein R₂₃ is selected from cycloalkyl, heterocycloalkyl, aryl, heteroaryl,

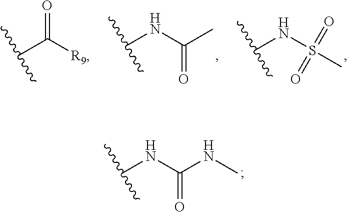

wherein R₉ is selected from H, alkyl, OH, NH₂, and alkoxy; or
(b) 2-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O, and X₂ may be further optionally substituted with 1 or 2 substituents R₂₉ and R₃₀, wherein R₂₉ and R₃₀ are independently selected from alkyl, cycloalkyl, alkoxy, OH, oxo, CF₃, and halo.

180. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof, wherein Formula (II) is represented by Formula (IIb),

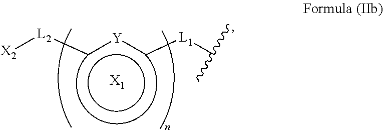

wherein:
n is 0 or 1;
L₁ is a linker selected from a single bond, —(CH₂)₁₋₃—, —(CH)(alkyl)-, and —C(alkyl)(alkyl)-; Y is selected from C, N, O and S;
X₁ is phenyl or 5- or 6-membered aromatic ring containing one, two or three ring members that are selected from N, S, and O; wherein X₁ may be optionally substituted with 1 or 2 substituents independently selected from alkyl, cycloalkyl, alkoxy, OH, CF₃, and halo;
L₂ is a linker selected from a single bond, —(CH₂)₁₋₃—, —(CH)(alkyl)-, —C(alkyl)(alkyl)-, —C(═O)—, and —SO₂—;and
X₂ is either:
(a) —NR₂₀R₂₁,
wherein R₂₀ is selected from H, alkyl, and —(CH₂)₀₋₃—R_{22;}
wherein R₂₁ is selected from H, alkyl, and —(CH₂)₀₋₃—R₂₃;
wherein R₂₂ is selected from cycloalkyl, heterocycloalkyl, aryl, heteroaryl,

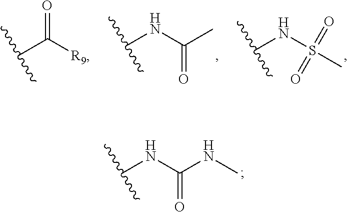

wherein R₂₃ is selected from cycloalkyl, heterocycloalkyl, aryl, heteroaryl,

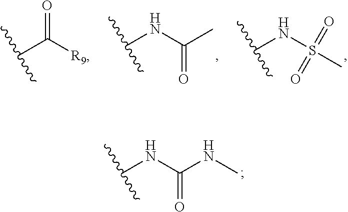

wherein R₉ is selected from H, alkyl, OH, NH₂, and alkoxy; or
(b) 2-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O.

181. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₂₁ is selected from alkyl and —(CH₂)₀₋₃—R_{23.}

182. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof, wherein, when X₂ is-NR₂₀R₂₁, L₂ is a linker selected from a single bond, —(CH₂)₁₋₃—, —(CH)(alkyl)-, —C(alkyl)(alkyl)-, and —SO₂—.

183. A compound according to numbered embodiment 182, or a tautomer, isomer, stereoisomer (including an enantiomer, a diastereoisomer and a racemic and scalemic mixture thereof), a deuterated analogue, and a pharmaceutically acceptable salt and/or solvate thereof, wherein, when X₂ is-NR₂₀R₂₁, L₂ is a linker selected from a single bond or-CH₂-.

184. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof, wherein, when X₂ is-NR₂₀R₂₁, X₁ is phenyl or pyridyl.

185. A compound according to any of numbered embodiments 1 to 181, or a tautomer, isomer, stereoisomer (including an enantiomer, a diastereoisomer and a racemic and scalemic mixture thereof), a deuterated analogue, and a pharmaceutically acceptable salt and/or solvate thereof, wherein X₂ is 2-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O, and X₂ may be further optionally substituted with 1 or 2 substituents R₂₉ and R₃₀, wherein R₂₉ and R₃₀ are independently selected from alkyl, cycloalkyl, alkoxy, OH, oxo, CF₃, and halo.

186. A compound according to any of numbered embodiments 1 to 181, or a tautomer, isomer, stereoisomer (including an enantiomer, a diastereoisomer and a racemic and scalemic mixture thereof), a deuterated analogue, and a pharmaceutically acceptable salt and/or solvate thereof, wherein X₂ is 2-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O.

187. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof, wherein Formula (II) is represented by Formula (IIa),

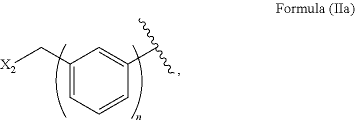

wherein:
n is 0 or 1;
X₂ is a non-aromatic, carbon-containing mono- or bi-cyclic ring containing 5, 6, or 7 ring members,
wherein at least one ring member is independently selected from N, S, and O;
wherein X₂ may be optionally substituted with 1 or 2 substituents R₁₈ and R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O.

188. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof,
wherein R₁₈ and R₁₉ are independently selected from alkyl, cycloalkyl, alkoxy, OH, CF₃, and halo.

189. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof,
wherein X₂ is a non-aromatic carbon-containing mono- or bi-cyclic ring containing 5, 6, or 7 ring members, wherein at least one ring member is N.

190. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof,
wherein X₂ is a non-aromatic carbon-containing mono- or bi-cyclic ring containing 5, 6, or 7 ring members, wherein at least one ring member is N, and wherein, when X₂ is a bi-cyclic ring system, the bicyclic ring system is fused.

191. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof,
wherein X₂ is a non-aromatic carbon-containing mono- or bi-cyclic ring containing 5 or 6 ring members, wherein at least one ring member is N.

192. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof,
wherein X₂ is a non-aromatic carbon-containing mono- or bi-cyclic ring containing 5 or 6 ring members, wherein at least one ring member is N, and wherein, when X₂ is a bi-cyclic ring system, the bicyclic ring system is fused.

193. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof,
wherein X₂ is a non-aromatic carbon-containing mono- or bi-cyclic ring containing 5, 6, or 7 ring members, wherein one ring member is N.

194. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof,
wherein X₂ is a non-aromatic carbon-containing mono- or bi-cyclic ring containing 5, 6, or 7 ring members, wherein one ring member is N, and wherein, when X₂ is a bi-cyclic ring system, the bicyclic ring system is fused.

195. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof,
wherein X₂ is a non-aromatic carbon-containing mono- or bi-cyclic ring containing 5 or 6 ring members, wherein one ring member is N.

196. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof,
wherein X₂ is a non-aromatic carbon-containing mono- or bi-cyclic ring containing 5 or 6 ring members, wherein one ring member is N, and wherein, when X₂ is a bi-cyclic ring system, the bicyclic ring system is fused.

197. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof,
wherein X₂ is attached to the remainder of the molecule via a N ring member of X_{2.}

198. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof,
wherein X₂ is selected from the structures

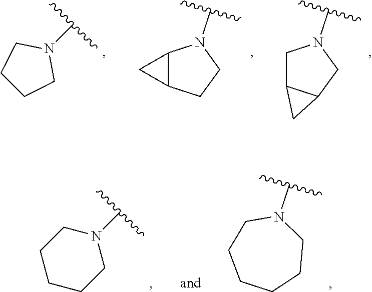 [SMILES: *N1CCCC1 ; *N1CCC2CC21 ; *N1CC2CC2C1 ; *N1CCCCC1 ; *N1CCCCCC1]

wherein X₂ may be optionally substituted with 1 or 2 substituents R₁₈ and R₁₉, wherein R₁₈ and R₁₉ are independently selected from alkyl, cycloalkyl, alkoxy, OH, oxo, CF₃, and halo,
or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O, and X₂ may be further optionally substituted with 1 or 2 substituents R₂₉ and R₃₀, wherein R₂₉ and R₃₀ are independently selected from alkyl, cycloalkyl, alkoxy, OH, oxo, CF₃, and halo.

199. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof,
wherein X₂ is selected from the structures and

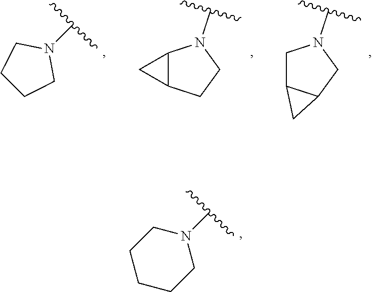 [SMILES: *N1CCCC1 ; *N1CCC2CC21 ; *N1CC2CC2C1 ; *N1CCCCC1]

wherein X₂ may be optionally substituted with 1 or 2 substituents R₁₈ and R₁₉, wherein R₁₈ and R₁₉ are independently selected from alkyl, cycloalkyl, alkoxy, OH, oxo, CF₃, and halo,
or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O, and X₂ may be further optionally substituted with 1 or 2 substituents R₂₉ and R₃₀, wherein R₂₉ and R₃₀ are independently selected from alkyl, cycloalkyl, alkoxy, OH, oxo, CF₃, and halo.

200. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof,
wherein X₂ is selected from the structures

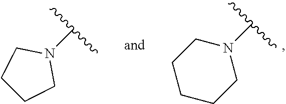 [SMILES: *N1CCCC1 ; *N1CCCCC1]

wherein X₂ may optionally be substituted with 1 or 2 substituents independently selected from alkyl, cyclopropyl, (C₁-C₆) alkoxy, OH, CN, CF₃, oxo, and halo.

201. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof,
wherein X₂ may optionally be substituted with 1 or 2 substituents independently selected from alkyl, cyclopropyl, (C₁-C₅) alkoxy, OH, CN, CF₃, and halo.

202. A compound according to any of numbered embodiments 1-200, or a tautomer, isomer, stereoisomer (including an enantiomer, a diastereoisomer and a racemic and scalemic mixture thereof), a deuterated analogue, and a pharmaceutically acceptable salt and/or solvate thereof,
wherein X₂ is selected from the structures

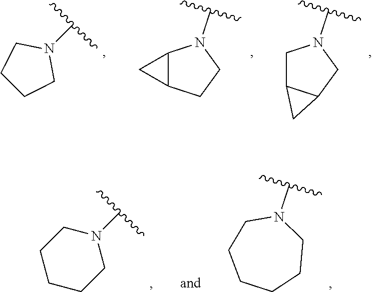

wherein X₂ is substituted with an oxo substituent, and X₂ may be optionally substituted with 1 or 2 substituents R₁₈ and R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O, and X₂ may be further optionally substituted with R₃₀, wherein R₃₀ is selected from alkyl, cycloalkyl, alkoxy, OH, oxo, CF₃, and halo.

203. A compound according to any of numbered embodiments 1-200, or a tautomer, isomer, stereoisomer (including an enantiomer, a diastereoisomer and a racemic and scalemic mixture thereof), a deuterated analogue, and a pharmaceutically acceptable salt and/or solvate thereof,
wherein X₂ is selected from the structures

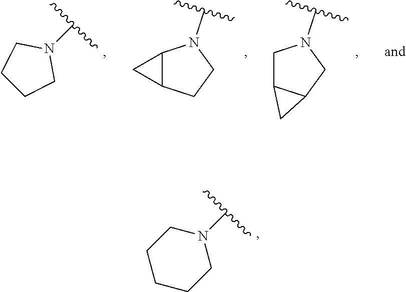

wherein X₂ is substituted with an oxo substituent, and X₂ may be optionally substituted with 1 or 2 substituents R₁₈ and R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O, and X₂ may be further optionally substituted with R₃₀, wherein R₃₀ is selected from alkyl, cycloalkyl, alkoxy, OH, oxo, CF₃, and halo.

204. A compound according to any of numbered embodiments 1-200, or a tautomer, isomer, stereoisomer (including an enantiomer, a diastereoisomer and a racemic and scalemic mixture thereof), a deuterated analogue, and a pharmaceutically acceptable salt and/or solvate thereof,
wherein X₂ is selected from the structures

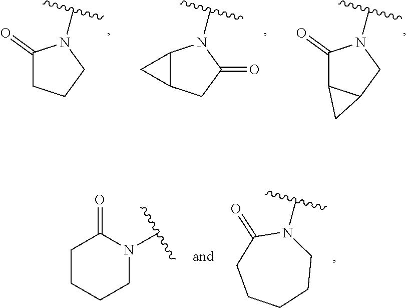 [SMILES: *N1CCCC1=O ; *N1C(=O)CC2CC21 ; *N1CC2CC2C1=O ; *N1CCCCC1=O ; *N1CCCCCC1=O]

wherein X₂ may be optionally substituted with 1 or 2 substituents R₁₈ and R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O, and X₂ may be further optionally substituted with R₃₀, wherein R₃₀ is selected from alkyl, cycloalkyl, alkoxy, OH, oxo, CF₃, and halo.

205. A compound according to any of numbered embodiments 1-200, or a tautomer, isomer, stereoisomer (including an enantiomer, a diastereoisomer and a racemic and scalemic mixture thereof), a deuterated analogue, and a pharmaceutically acceptable salt and/or solvate thereof,
wherein X₂ is selected from the structures

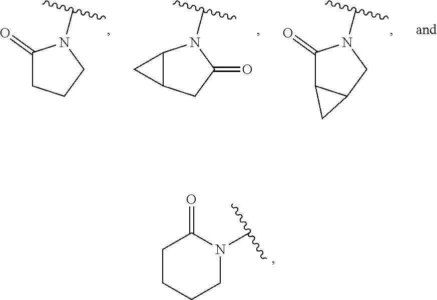 [SMILES: *N1CCCC1=O ; *N1C(=O)CC2CC21 ; *N1CC2CC2C1=O ; *N1CCCCC1=O]

wherein X₂ may be optionally substituted with 1 or 2 substituents R₁₈ and R₁₉, wherein R₁₈ and R₁₉ are independently selected from alkyl, cycloalkyl, alkoxy, OH, oxo, CF₃, and halo,
or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O, and X₂ may be further optionally substituted with 1 or 2 substituents R₂₉ and R₃₀, wherein R₂₉ and R₃₀ are independently selected from alkyl, cycloalkyl, alkoxy, OH, oxo, CF₃, and halo.

206. A compound according to any of the previous numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof,
wherein X₂ is not

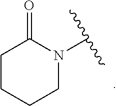 [SMILES: *N1CCCCC1=O]

207. A compound according to any of the previous numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof,
wherein X₂ is a cyclic amide.

208. A compound according to any of the previous numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof,
wherein X₂ is not a cyclic amide.

209. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof,
wherein n is 1.

210. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof, wherein L₃ is a linker selected from a single bond and —(CH₂)₁₋₃ —.

211. A compound according to numbered embodiment 210, or a tautomer, isomer, stereoisomer (including an enantiomer, a diastereoisomer and a racemic and scalemic mixture thereof), a deuterated analogue, and a pharmaceutically acceptable salt and/or solvate thereof, wherein L₃ is a single bond.

212. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof, wherein A does not contain both N and S ring members.

213. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof, wherein A is phenyl or a 5- or 6-membered carbon-containing aromatic ring containing one, two or three ring members that are selected from N and O.

214. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof, wherein A is phenyl or a 6-membered carbon-containing aromatic ring containing one, two or three nitrogen ring members.

215. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof, wherein A is phenyl, pyridyl, or pyrimidyl.

216. A compound according to numbered embodiment 215, or a tautomer, isomer, stereoisomer (including an enantiomer, a diastereoisomer and a racemic and scalemic mixture thereof), a deuterated analogue, and a pharmaceutically acceptable salt and/or solvate thereof, wherein A is phenyl, pyridyl, or pyrimidyl, wherein A is not further substituted with any optional substituents.

217. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof, wherein A is phenyl or pyridyl.

218. A compound according to numbered embodiment 217, or a tautomer, isomer, stereoisomer (including an enantiomer, a diastereoisomer and a racemic and scalemic mixture thereof), a deuterated analogue, and a pharmaceutically acceptable salt and/or solvate thereof, wherein A is phenyl.

219. A compound according to numbered embodiment 217, or a tautomer, isomer, stereoisomer (including an enantiomer, a diastereoisomer and a racemic and scalemic mixture thereof), a deuterated analogue, and a pharmaceutically acceptable salt and/or solvate thereof, wherein A is pyridyl.

220. A compound according to any of numbered embodiments 1-215 or 217-219, or a tautomer, isomer, stereoisomer (including an enantiomer, a diastereoisomer and a racemic and scalemic mixture thereof), a deuterated analogue, and a pharmaceutically acceptable salt and/or solvate thereof, wherein A is optionally substituted with a single substituent, wherein the substituent is a halo substituent.

221. A compound according to any of numbered embodiments 1-215 or 217-220, or a tautomer, isomer, stereoisomer (including an enantiomer, a diastereoisomer and a racemic and scalemic mixture thereof), a deuterated analogue, and a pharmaceutically acceptable salt and/or solvate thereof, wherein A is optionally substituted with a single substituent, wherein the substituent is F.

222. A compound according to any of numbered embodiments 1-215 or 217-221, or a tautomer, isomer, stereoisomer (including an enantiomer, a diastereoisomer and a racemic and scalemic mixture thereof), a deuterated analogue, and a pharmaceutically acceptable salt and/or solvate thereof, wherein A is substituted with a single substituent, wherein the substituent is a halo substituent.

223. A compound according to any of numbered embodiments 1-215 or 217-222, or a tautomer, isomer, stereoisomer (including an enantiomer, a diastereoisomer and a racemic and scalemic mixture thereof), a deuterated analogue, and a pharmaceutically acceptable salt and/or solvate thereof, wherein A is substituted with a single substituent, wherein the substituent is F.

224. A compound according to any of numbered embodiments 1-219, or a tautomer, isomer, stereoisomer (including an enantiomer, a diastereoisomer and a racemic and scalemic mixture thereof), a deuterated analogue, and a pharmaceutically acceptable salt and/or solvate thereof, wherein A is not substituted with any optional substituents.

225. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate

thereof, wherein, when A′ is a cyclic amide, A′ is selected from

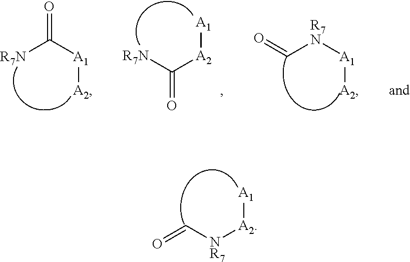

226. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof, wherein, when A′ is a cyclic amide, A′ is selected from

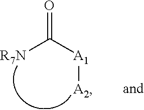

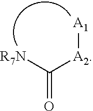

227. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof, wherein A′ is a cyclic amide, and A′ is selected from

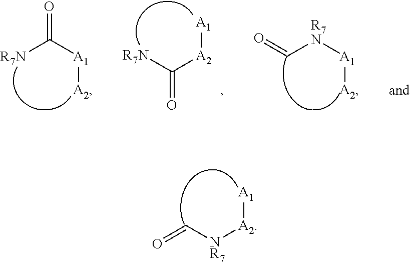

228. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof, wherein A′ is a cyclic amide, and A′ is selected from

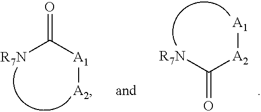

229. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof,
wherein A′ is a non-aromatic carbon-containing monocyclic ring containing 5, 6, or 7 ring members, wherein at least one ring member is N.

230. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof,
wherein A′ is a non-aromatic carbon-containing monocyclic ring containing 6 or 7 ring members, wherein at least one ring member is N.

231. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof,
wherein A′ is a non-aromatic carbon-containing monocyclic ring containing 6 ring members,
wherein at least one ring member is N.

232. A compound according to any of numbered embodiments 1-229, or a tautomer, isomer, stereoisomer (including an enantiomer, a diastereoisomer and a racemic and scalemic mixture thereof), a deuterated analogue, and a pharmaceutically acceptable salt and/or solvate thereof,
wherein A′ is a non-aromatic carbon-containing monocyclic ring containing 5, 6, or 7 ring members, wherein one ring member is N.

233. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof,
wherein A′ is a non-aromatic carbon-containing monocyclic ring containing 6 ring members,
wherein one ring member is N.

234. A compound according to any of numbered embodiments 1-230 or 232, or a tautomer, isomer, stereoisomer (including an enantiomer, a diastereoisomer and a racemic and scalemic mixture thereof), a deuterated analogue, and a pharmaceutically acceptable salt and/or solvate thereof,
wherein A′ is a non-aromatic carbon-containing monocyclic ring containing 7 ring members,
wherein one ring member is N or wherein one ring member is N and a second ring member is O.

235. A compound according to any of numbered embodiments 1-230 or 232, or a tautomer, isomer, stereoisomer (including an enantiomer, a diastereoisomer and a racemic and scalemic mixture thereof), a deuterated analogue, and a pharmaceutically acceptable salt and/or solvate thereof,
wherein A′ is a non-aromatic carbon-containing monocyclic ring containing 7 ring members,
wherein one ring member is N.

236. A compound according to any of numbered embodiments 1-230, 232, or 234, or a tautomer, isomer, stereoisomer (including an enantiomer, a diastereoisomer and a racemic and scalemic mixture thereof), a deuterated analogue, and a pharmaceutically acceptable salt and/or solvate thereof,
wherein A′ is a non-aromatic carbon-containing monocyclic ring containing 7 ring members,
wherein one ring member is N and a second ring member is O.

237. A compound according to any one of numbered embodiments 1-230, or a tautomer, isomer, stereoisomer (including an enantiomer, a diastereoisomer and a racemic and scalemic mixture thereof), a deuterated analogue, and a pharmaceutically acceptable salt and/or solvate thereof,
wherein A′ is selected from

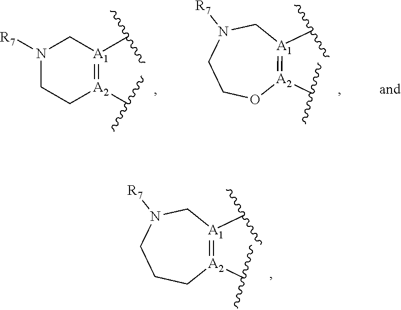

and wherein A′ may be optionally substituted with 1, 2, or 3 further substituents R₁₀, R₁₁, and R₁₇, wherein R₁₀, R₁₁, and R₁₇, are independently selected from alkyl, cycloalkyl, alkoxy, OH, oxo, CF₃, and halo, or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O, or wherein A′ may be optionally substituted with 1 or 2 further substituents R₄₃ and R₄₄ independently selected from alkyl, cycloalkyl, alkoxy, OH, oxo, CF₃, and halo, and two different carbon ring members within A′ may be connected by a bridging —CH₂— group.

238. A compound according to any one of numbered embodiments 1-230, or a tautomer, isomer, stereoisomer (including an enantiomer, a diastereoisomer and a racemic and scalemic mixture thereof), a deuterated analogue, and a pharmaceutically acceptable salt and/or solvate thereof,
wherein A′ is selected from

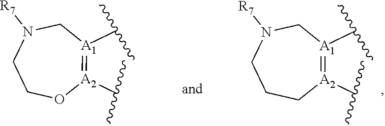

and wherein A′ may be optionally substituted with 1, 2, or 3 further substituents R₁₀, R₁₁, and R₁₇, wherein R₁₀, R₁₁, and R₁₇, are independently selected from alkyl, cycloalkyl, alkoxy, OH, oxo, CF₃, and halo, or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O, or wherein A′ may be optionally substituted with 1 or 2 further substituents R₄₃ and R₄₄ independently selected from alkyl, cycloalkyl, alkoxy, OH, oxo, CF₃, and halo, and two different carbon ring members within A′ may be connected by a bridging —CH₂— group.

239. A compound according to any one of numbered embodiments 1-230, or a tautomer, isomer, stereoisomer (including an enantiomer, a diastereoisomer and a racemic and scalemic mixture thereof), a deuterated analogue, and a pharmaceutically acceptable salt and/or solvate thereof,
wherein A′ is selected from

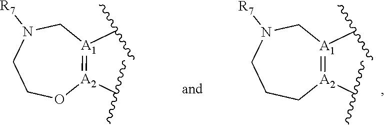

and wherein A′ may be optionally substituted with 1, 2, or 3 further substituents R₁₀, R₁₁, and R₁₇, wherein R₁₀, R₁₁, and R₁₇, are independently selected from alkyl, cycloalkyl, alkoxy, OH, oxo, CF₃, and halo, or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O.

240. A compound according to numbered embodiment 237, or a tautomer, isomer, stereoisomer (including an enantiomer, a diastereoisomer and a racemic and scalemic mixture thereof), a deuterated analogue, and a pharmaceutically acceptable salt and/or solvate thereof,
wherein A′is selected from

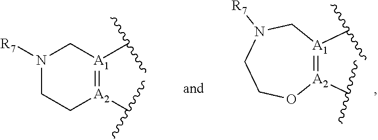

and wherein A′ may be optionally substituted with 1, 2, or 3 further substituents R₁₀, R₁₁, and R₁₇, wherein R₁₀, R₁₁, and R₁₇, are independently selected from alkyl, cycloalkyl, alkoxy, OH, oxo, CF₃, and halo, or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O, or wherein A′ may be optionally substituted with 1 or 2 further substituents R₄₃ and R₄₄ independently selected from alkyl, cycloalkyl, alkoxy, OH, oxo, CF₃, and halo, and two different carbon ring members within A′ may be connected by a bridging —CH₂-group.

241. A compound according to numbered embodiment 240, or a tautomer, isomer, stereoisomer (including an enantiomer, a diastereoisomer and a racemic and scalemic mixture thereof), a deuterated analogue, and a pharmaceutically acceptable salt and/or solvate thereof, wherein A′ is

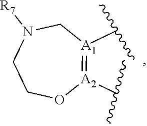

and wherein A′ may be optionally substituted with 1, 2, or 3 further substituents R₁₀, R₁₁, and R₁₇, wherein R₁₀, R₁₁, and R₁₇, are independently selected from alkyl, cycloalkyl, alkoxy, OH, oxo, CF₃, and halo, or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O, or wherein A′ may be optionally substituted with 1 or 2 further substituents R₄₃ and R₄₄ independently selected from alkyl, cycloalkyl, alkoxy, OH, oxo, CF₃, and halo, and two different carbon ring members within A′ may be connected by a bridging —CH₂-group.

242. A compound according to numbered embodiment 239, or a tautomer, isomer, stereoisomer (including an enantiomer, a diastereoisomer and a racemic and scalemic mixture thereof), a deuterated analogue, and a pharmaceutically acceptable salt and/or solvate thereof, wherein A′ is

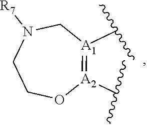

and wherein A′ may be optionally substituted with 1, 2, or 3 further substituents R₁₀, R₁₁, and R₁₇, wherein R₁₀, R₁₁, and R₁₇, are independently selected from alkyl, cycloalkyl, alkoxy, OH, oxo, CF₃, and halo, or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and 0.

243. A compound according to any of the preceding embodiments, or a tautomer, isomer, stereoisomer (including an enantiomer, a diastereoisomer and a racemic and scalemic mixture thereof), a deuterated analogue, and a pharmaceutically acceptable salt and/or solvate thereof, wherein A′ is further substituted with an oxo substituent.

244. A compound according to numbered embodiment 237, or a tautomer, isomer, stereoisomer (including an enantiomer, a diastereoisomer and a racemic and scalemic mixture thereof), a deuterated analogue, and a pharmaceutically acceptable salt and/or solvate thereof, wherein A′ is selected from

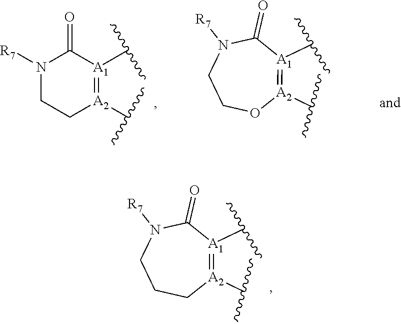

and wherein A′ may be optionally substituted with 1 or 2 further substituents R₁₀ and R₁₁, wherein R₁₀ and R₁₁ are independently selected from alkyl, cycloalkyl, alkoxy, OH, oxo, CF₃, and halo, or wherein A′ may be optionally substituted with 1 further substituent R₂₄ selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O, or wherein A′ may be optionally substituted with 1 further substituent R₄₃ selected from alkyl, cycloalkyl, alkoxy, OH, oxo, CF₃, and halo, and two different carbon ring members within A′ may be connected by a bridging —CH₂-group.

245. A compound according to numbered embodiment 244, or a tautomer, isomer, stereoisomer (including an enantiomer, a diastereoisomer and a racemic and scalemic mixture thereof), a deuterated analogue, and a pharmaceutically acceptable salt and/or solvate thereof, wherein A′ is selected from

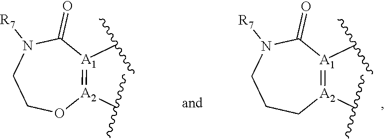

and wherein A′ may be optionally substituted with 1 or 2 further substituents R₁₀ and R₁₁, wherein R₁₀ and R₁₁ are independently selected from alkyl, cycloalkyl, alkoxy, OH, oxo, CF₃, and halo, or wherein A′ may be optionally substituted with 1 further substituent R₂₄ selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O, or wherein A′ may be optionally substituted with 1 further substituent R₄₃ selected from alkyl, cycloalkyl, alkoxy, OH, oxo, CF₃, and halo, and two different carbon ring members within A′ may be connected by a bridging —CH₂-group.

246. A compound according to numbered embodiment 239, or a tautomer, isomer, stereoisomer (including an enantiomer, a diastereoisomer and a racemic and scalemic mixture thereof), a deuterated analogue, and a pharmaceutically acceptable salt and/or solvate thereof, wherein A′ is selected from

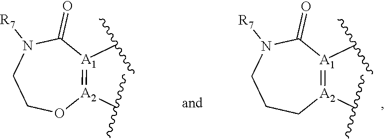

and wherein A′ may be optionally substituted with 1 or 2 further substituents R₁₀ and R₁₁, wherein R₁₀ and R₁₁ are independently selected from alkyl, cycloalkyl, alkoxy, OH, oxo, CF₃, and halo, or wherein A′ may be optionally substituted with 1 further substituent R₂₄ selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O.

247. A compound according to numbered embodiment 244, or a tautomer, isomer, stereoisomer (including an enantiomer, a diastereoisomer and a racemic and scalemic mixture thereof), a deuterated analogue, and a pharmaceutically acceptable salt and/or solvate thereof, wherein A′ is selected from

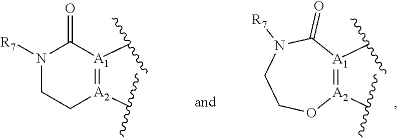

and wherein A′ may be optionally substituted with 1 or 2 further substituents R₁₀ and R₁₁, wherein R₁₀ and R₁₁ are independently selected from alkyl, cycloalkyl, alkoxy, OH, oxo, CF₃, and halo, or wherein A′ may be optionally substituted with 1 further substituent R₂₄ selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O, or wherein A′ may be optionally substituted with 1 further substituent R₄₃ selected from alkyl, cycloalkyl, alkoxy, OH, oxo, CF₃, and halo, and two different carbon ring members within A′ may be connected by a bridging —CH₂-group.

248. A compound according to numbered embodiment 247, or a tautomer, isomer, stereoisomer (including an enantiomer, a diastereoisomer and a racemic and scalemic mixture thereof), a deuterated analogue, and a pharmaceutically acceptable salt and/or solvate thereof, wherein A′ is

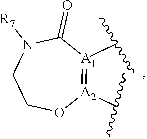

and wherein A′ may be optionally substituted with 1 or 2 further substituents R₁₀ and R₁₁, wherein R₁₀ and R₁₁ are independently selected from alkyl, cycloalkyl, alkoxy, OH, oxo, CF₃, and halo, or wherein A′ may be optionally substituted with 1 further substituent R₂₄ selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O, or wherein A′ may be optionally substituted with 1 further substituent R₄₃ selected from alkyl, cycloalkyl, alkoxy, OH, oxo, CF₃, and halo, and two different carbon ring members within A′ may be connected by a bridging —CH₂-group.

249. A compound according to numbered embodiment 246, or a tautomer, isomer, stereoisomer (including an enantiomer, a diastereoisomer and a racemic and scalemic mixture thereof), a deuterated analogue, and a pharmaceutically acceptable salt and/or solvate thereof, wherein A′ is

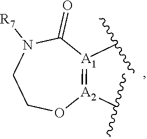

and wherein A′ may be optionally substituted with 1 or 2 further substituents R₁₀ and R₁₁, wherein R₁₀ and R₁₁ are independently selected from alkyl, cycloalkyl, alkoxy, OH, oxo, CF₃, and halo, or wherein A′ may be optionally substituted with 1 further substituent R₂₄ selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O.

250. A compound according to any of the preceding embodiments, or a tautomer, isomer, stereoisomer (including an enantiomer, a diastereoisomer and a racemic and scalemic mixture thereof), a deuterated analogue, and a pharmaceutically acceptable salt and/or solvate thereof, wherein A′ is selected from

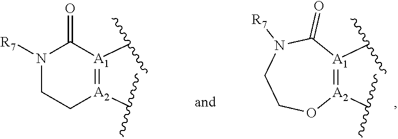

and wherein A′ has no further substituents.

251. A compound according to numbered embodiment 250, or a tautomer, isomer, stereoisomer (including an enantiomer, a diastereoisomer and a racemic and scalemic mixture thereof), a deuterated analogue, and a pharmaceutically acceptable salt and/or solvate thereof, wherein A′ is

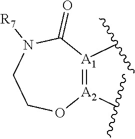

and has no further substituents.

252. A compound according to any of the preceding numbered embodimentsor a tautomer, isomer, stereoisomer (including an enantiomer, a diastereoisomer and a racemic and scalemic mixture thereof), a deuterated analogue, and a pharmaceutically acceptable salt and/or solvate thereof,
wherein R₇, when present, is bonded to A′ via a N ring member.

253. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof,
wherein R₇ is present.

254. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof,
wherein R₇ is alkyl.

255. A compound according to numbered embodiment 254, or a tautomer, isomer, stereoisomer (including an enantiomer, a diastereoisomer and a racemic and scalemic mixture thereof), a deuterated analogue, and a pharmaceutically acceptable salt and/or solvate thereof,
wherein R₇ is (C₁-C₃)alkyl.

256. A compound according to numbered embodiment 255, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₇ is (C₁-C₂)alkyl.

257. A compound according to numbered embodiment 256, or a tautomer, isomer, wherein R₇ is (C₂)alkyl.

258. A compound according to numbered embodiment 257, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₇ is (C₂)alkyl, wherein in the (C₂)alkyl is unsubstituted or is substituted with OH or halo substituents.

259. A compound according to numbered embodiment 255, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₇ is selected from —CH₃, —CH₂—CH₃, —CH₂—CH₂—OH, CH₂—CHF₂, CH₂—CF₃, CH₂—CH₂—CH₂—OH, —CH(CH₃)₂, and —CH₂—(C═O)—NH_{2.}

260. A compound according to numbered embodiment 255, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₇ is selected from —CH₃, —CH₂—CH₃, —CH₂—CH₂—OH, CH₂—CHF₂, CH₂—CF₃, CH₂—CH₂—CH₂—OH, and —CH(CH₃)_{2.}

261. A compound according to numbered embodiment 260, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₇ is selected from —CH₃, —CH₂—CH₃ and CH₂—CH₂—OH.

262. A compound according to numbered embodiment 260, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₇ is selected from —CH₂—CH₃ and CH₂—CH₂—OH, CH₂—CHF₂, CH₂—CF₃, —CH₂—CH₂-CH₂—OH, and —CH (CH₃) 2.

263. A compound according to numbered embodiment 261, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₇ is selected from —CH₂—CH₃ and CH₂—CH₂—OH.

264. A compound according to numbered embodiment 261, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₇ is selected from —CH₃ and CH₂—CH₂—OH.

265. A compound according to numbered embodiment 263 or numbered embodiment 264, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₇ is CH₂—CH₂—OH.

266. A compound according to numbered embodiment 264, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₇ is —CH_{3.}

267. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₁₀ R₁₁, and R₁₇ are absent.

268. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₁₀ and R₁₁ are absent.

269. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₂₄, R₂₅, R₂₆, and R₂₇ are absent.

270. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₂₄, R₂₅, and R₂₆ are absent.

271. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₄₃ and R₄₄ are absent.

272. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof, wherein A′ is not substituted with further substituents beyond R_{7.}

273. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof,
wherein Formula (III) is represented by Formula (IIIa), Formula (IIIb), Formula (IIIc), and Formula (IIId),

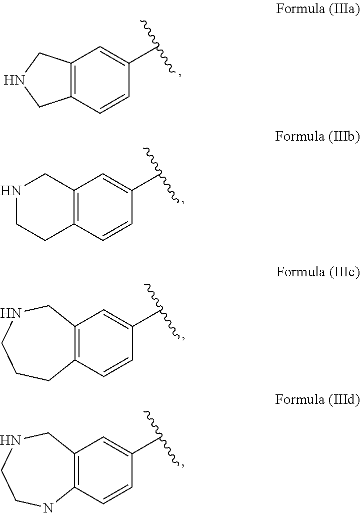

wherein:
A is a phenyl or pyridyl ring and A may be optionally substituted with 1 or 2 substituents independently selected from alkyl, cyclopropyl, alkoxy, OH, CF₃, and halo;

A′ is a fused pyrrolidine, piperidine, azepane, or oxazepane ring;
R₇, when present, is bonded to A′ via the N ring member of Formula (IIIa), Formula (IIIb), Formula (IIIc), or Formula (IIId), replacing H;

A′ may be optionally substituted with 1, 2, or 3 further substituents R₁₀, R₁₁, and R₁₇, wherein R_{10,}
R₁₁, and R₁₇, are independently selected from small alkyl, cyclopropyl, small alkoxy, OH, oxo, CF₃, and halo,
or A′ may be optionally substituted with 1 or 2 further substituents R₂₄ and R₂₇ independently selected from small alkyl, cyclopropyl, small alkoxy, OH, oxo, CF₃, and halo, and 2 further substituents R₂₅ and R₂₆, wherein R₂₅ and R₂₆ together with a carbon atom to which they are both attached form a 3-, 4-, or 5-membered non-aromatic monocyclic carbon-containing ring optionally wherein one or two ring members are selected from N, S, and O,
or A′ may be optionally substituted with 1 or 2 further substituents R₄₃ and R₄₄ independently selected from alkyl, cycloalkyl, alkoxy, OH, oxo, CF₃, and halo, and two different carbon ring members within A′ may be connected by a bridging —CH₂-group.

274. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof,
wherein Formula (III) is represented by Formula (IIIa), Formula (IIIb), and Formula (IIIc),

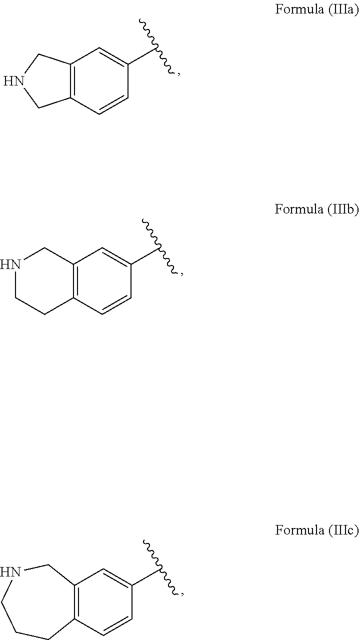

wherein:
A is a phenyl ring and A may be optionally substituted with 1 or 2 substituents independently selected from alkyl, cyclopropyl, alkoxy, OH, CF₃, and halo;

A′ is a fused pyrrolidine, piperidine or azepane ring;
R₇, when present, is bonded to A′ via the N ring member of Formula (IIIa), Formula (IIIb), or Formula (IIIc), replacing H;

A′ may be optionally substituted with 1, 2, or 3 further substituents R₁₀, R₁₁, and R₁₇, wherein R₁₀, R₁₁, and R₁₇, are independently selected from small alkyl, cyclopropyl, small alkoxy, OH, oxo, CF₃, and halo,
or A′ may be optionally substituted with 1 or 2 further substituents R₂₄ and R₂₇ independently selected from small alkyl, cyclopropyl, small alkoxy, OH, oxo, CF₃, and halo, and 2 further substituents R₂₅ and R₂₆, wherein R₂₅ and R₂₆ together with a carbon atom to which they are both attached form a 3-, 4-, or 5-membered non-aromatic monocyclic carbon-containing ring optionally wherein one or two ring members are selected from N, S, and O.

275. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof,
wherein Formula (III) is represented by Formula (IIIa), Formula (IIIb), and Formula (IIIc),

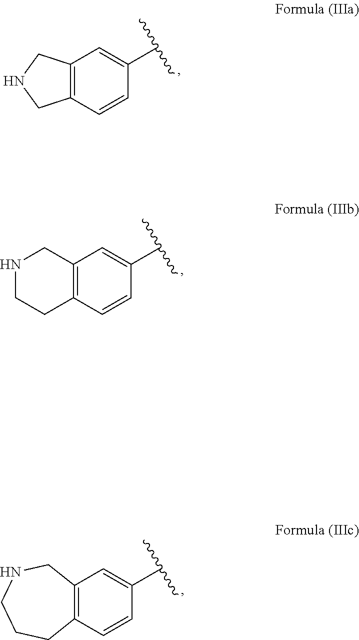

wherein:
A is a phenyl ring and A may be optionally substituted with 1 or 2 substituents independently selected from alkyl, cyclopropyl, alkoxy, OH, CF₃, and halo;

A′ is a fused pyrrolidine, piperidine or azepane ring;
R₇, when present, is bonded to A′ via the N ring member of Formula (IIIa), Formula (IIIb), or Formula (IIIc), replacing H;

A′ may be optionally substituted with 1 or 2 further substituents R₁₀ and R₁₁, wherein R₁₀ and R₁₁ are independently selected from small alkyl, cyclopropyl, small alkoxy, OH, oxo, CF₃, and halo, or A′ may be optionally substituted with 1 further substituents R₂₄ selected from small alkyl, cyclopropyl, small alkoxy, OH, oxo, CF₃, and halo, and 2 further substituents R₂₅ and R₂₆, wherein R₂₅ and R₂₆ together with a carbon atom to which they are both attached form a 3-, 4-, or 5-membered non-aromatic monocyclic carbon-containing ring optionally wherein one or two ring members are selected from N, S, and O.

276. A compound according to numbered embodiment 273, or a tautomer, isomer, stereoisomer (including an enantiomer, a diastereoisomer and a racemic and scalemic mixture thereof), a deuterated analogue, and a pharmaceutically acceptable salt and/or solvate thereof, wherein, when A′ is oxo-substituted, the oxo group is at the position labelled with a (*):

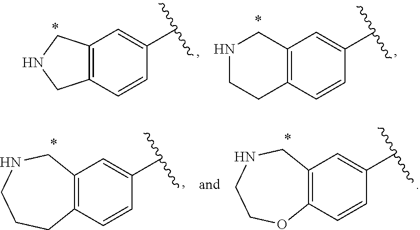 [SMILES: *c1ccc2c(c1)CNC2 ; *c1ccc2c(c1)CNCC2 ; *c1ccc2c(c1)CNCCC2 ; *c1ccc2c(c1)CNCCO2]

277. A compound according to numbered embodiment 274 or 275, or a tautomer, isomer, stereoisomer (including an enantiomer, a diastereoisomer and a racemic and scalemic mixture thereof), a deuterated analogue, and a pharmaceutically acceptable salt and/or solvate thereof, wherein, when A′ is oxo-substituted, the oxo group is at the position labelled with a (*):

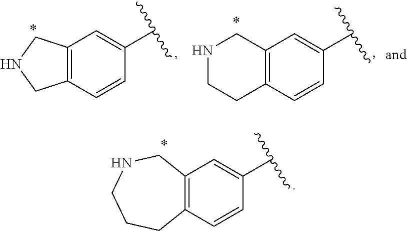 [SMILES: *c1ccc2c(c1)CNC2 ; *c1ccc2c(c1)CNCC2 ; *c1ccc2c(c1)CNCCC2]

278. A compound according to numbered embodiment 273 or 276, or a tautomer, isomer, stereoisomer (including an enantiomer, a diastereoisomer and a racemic and scalemic mixture thereof), a deuterated analogue, and a pharmaceutically acceptable salt and/or solvate thereof, wherein Formula (III) is represented by Formula (IIIb), Formula (IIIc), or Formula (IIId).

279. A compound according to any of numbered embodiments 273 to 278, or a tautomer, isomer, stereoisomer (including an enantiomer, a diastereoisomer and a racemic and scalemic mixture thereof), a deuterated analogue, and a pharmaceutically acceptable salt and/or solvate thereof, wherein Formula (III) is represented by Formula (IIIb) or Formula (IIId).

280. A compound according to any of numbered embodiments 273 to 279, or a tautomer, isomer, stereoisomer (including an enantiomer, a diastereoisomer and a racemic and scalemic mixture thereof), a deuterated analogue, and a pharmaceutically acceptable salt and/or solvate thereof, wherein Formula (III) is represented by Formula (IIIb).

281. A compound according to any of numbered embodiments 273, 276, or 278, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₁ is Formula (IIIa), Formula (IIIb), Formula (IIIc), or Formula (IIId).

282. A compound according to any of numbered embodiments 273 to 277, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₁ is Formula (IIIa), Formula (IIIb), or Formula (IIIc).

283. A compound according to numbered embodiment 281, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₁ is Formula (IIIb), Formula (IIIc), or Formula (IIId).

284. A compound according to numbered embodiment 283, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₁ is Formula (IIIb) or Formula (IIId).

285. A compound according to any of numbered embodiments 273-284, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₁ is Formula (IIIb).

286. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof, wherein Formula (III) is represented by Formula (IIIe),

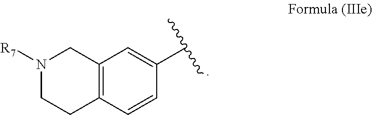

287. A compound according to numbered embodiment 286, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₁ is Formula (IIIe).

288. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₈ is selected from cycloalkyl, heterocycloalkyl,

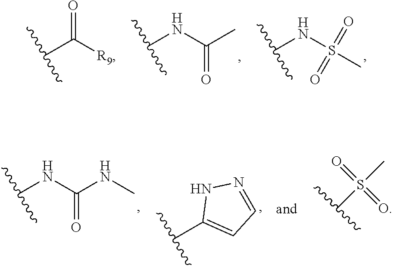

289. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₅ is selected from cycloalkyl, heterocycloalkyl,

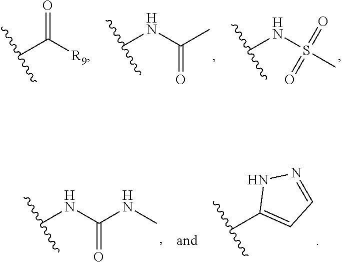

290. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₁ is selected from:

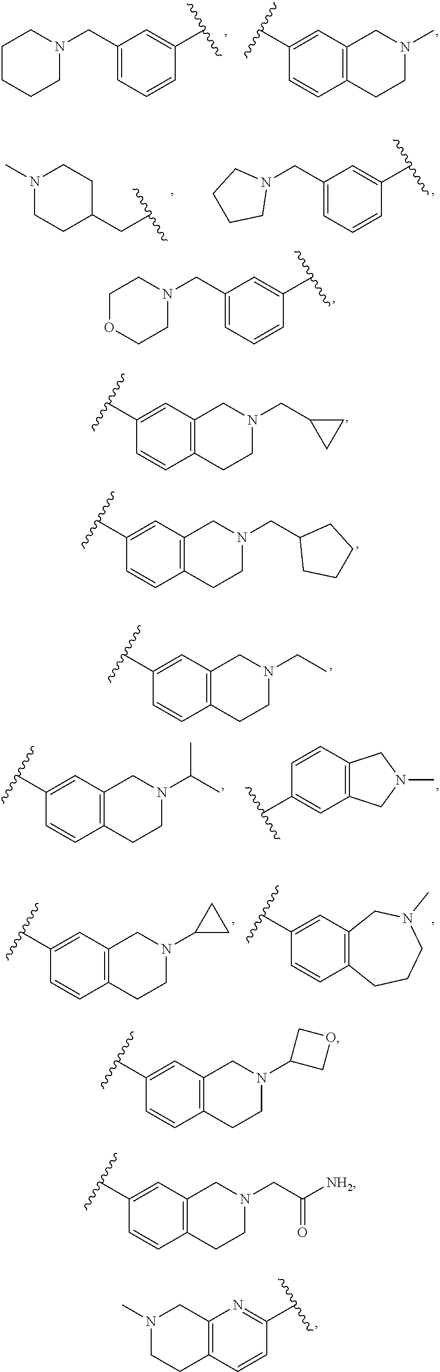

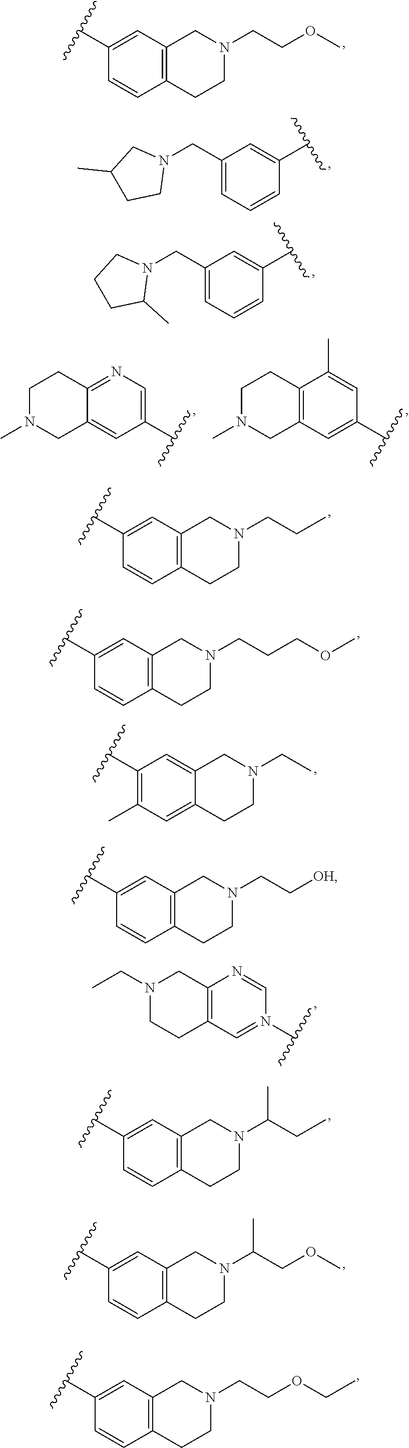

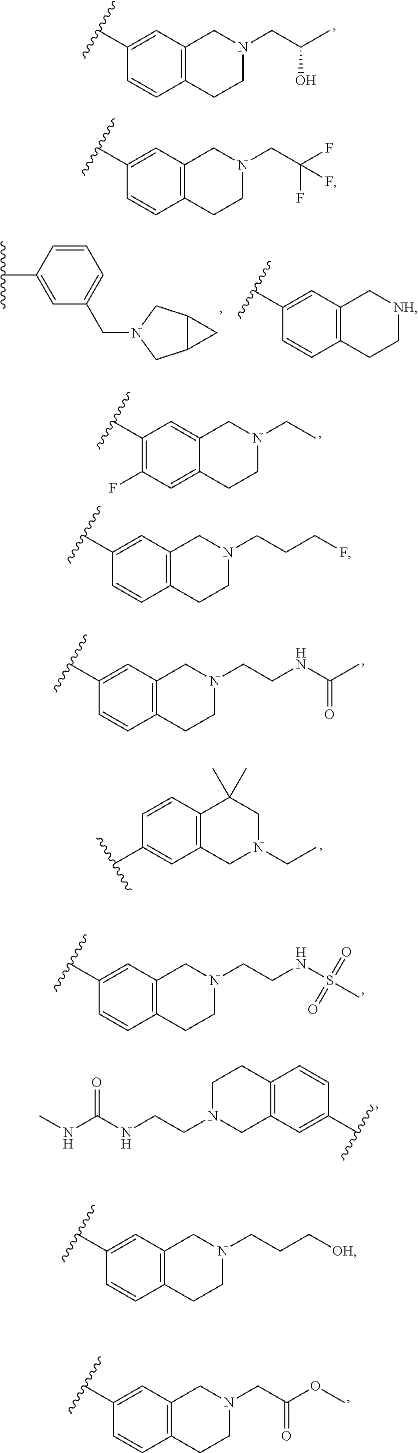

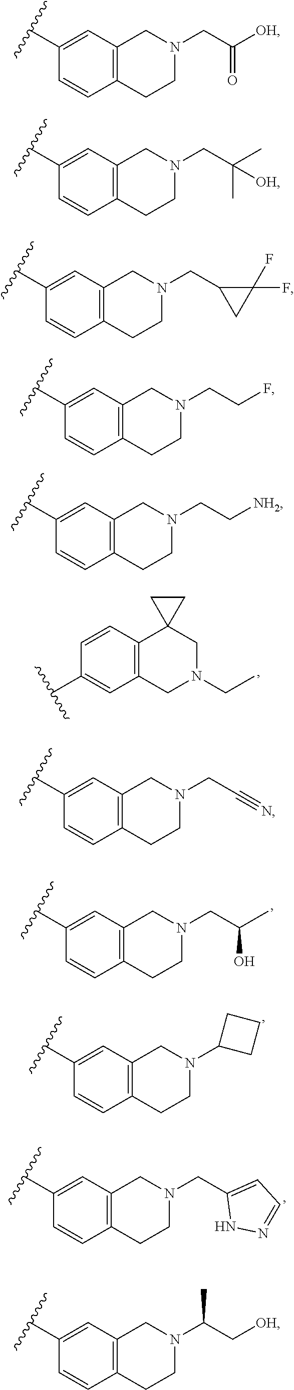

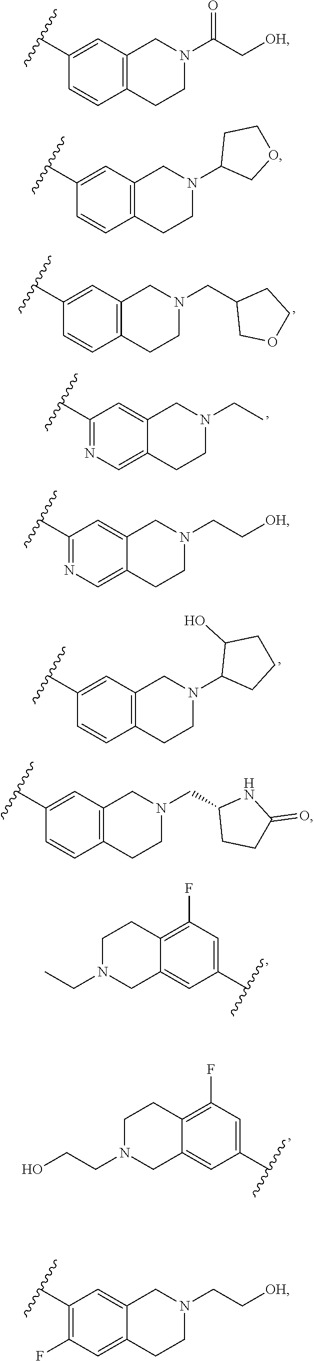

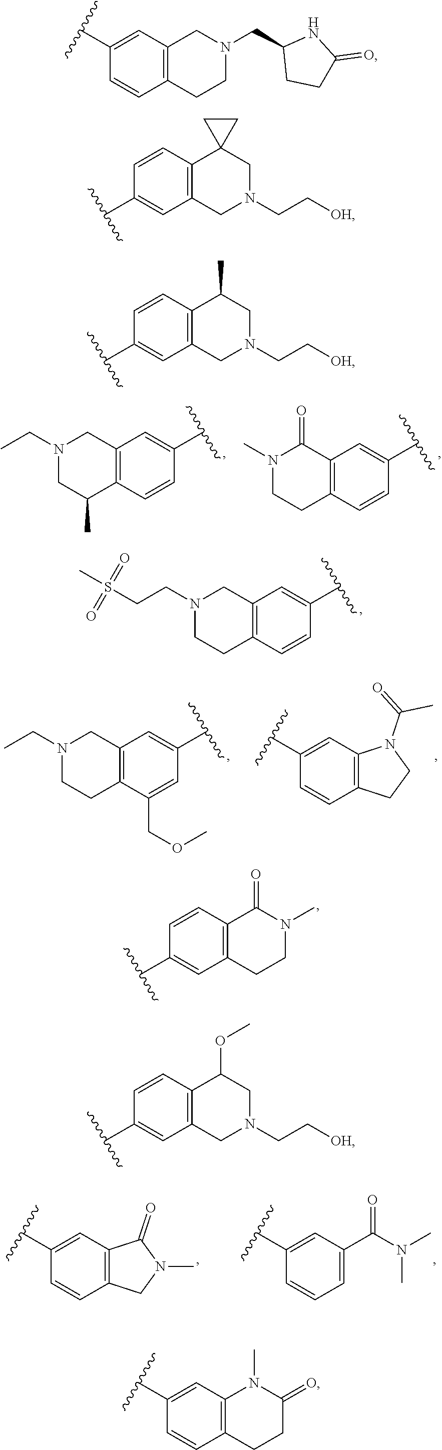

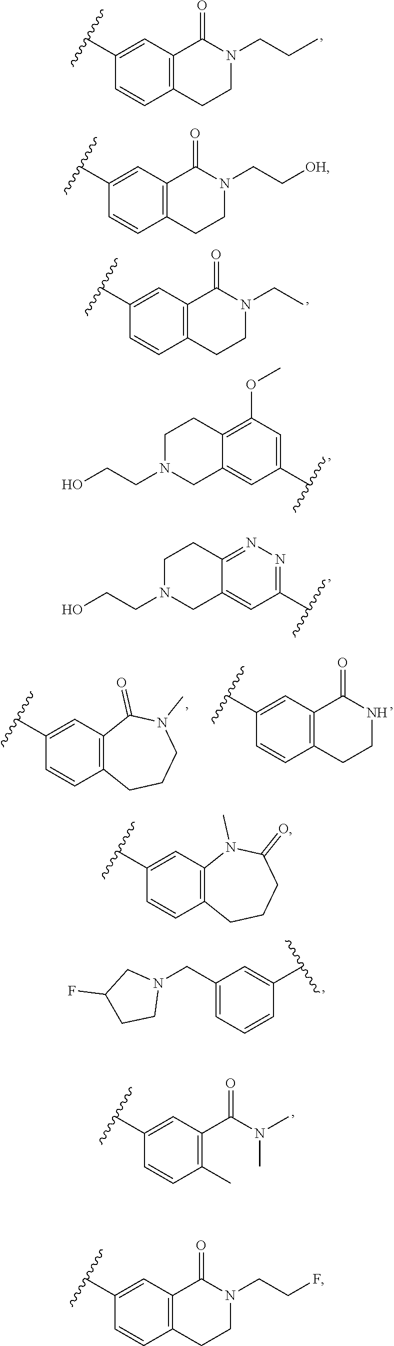

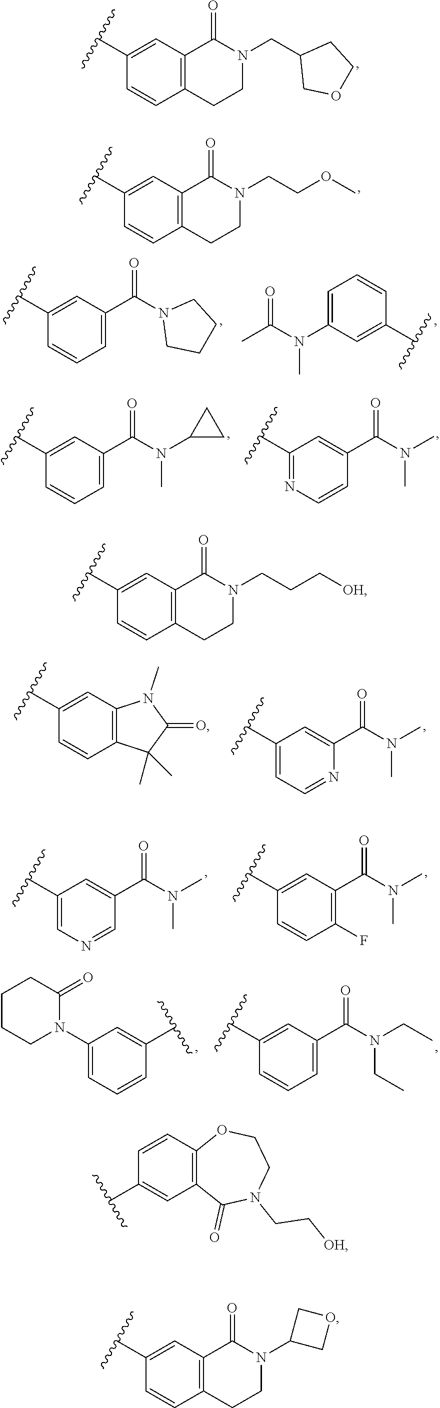

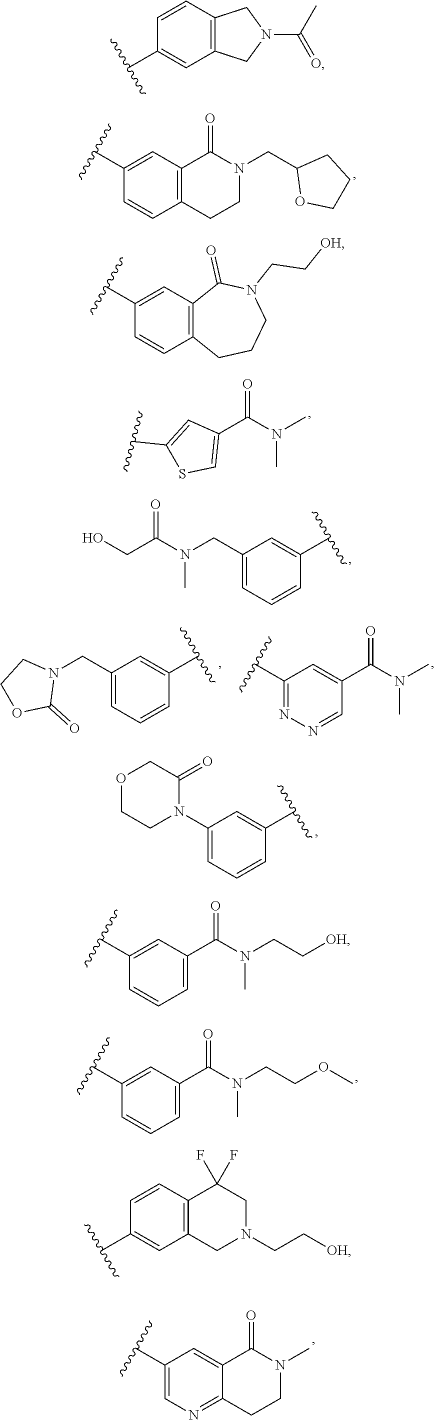

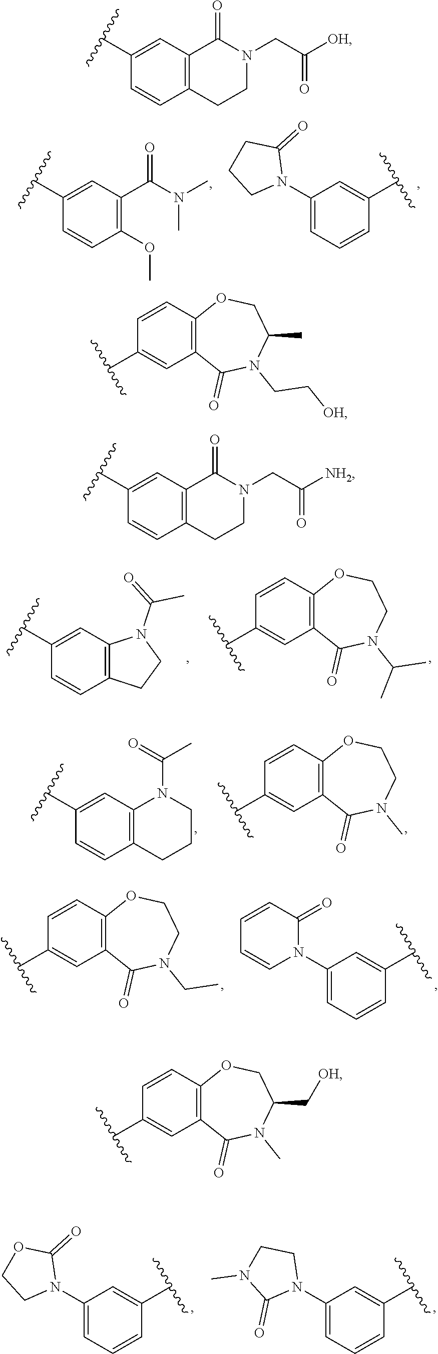

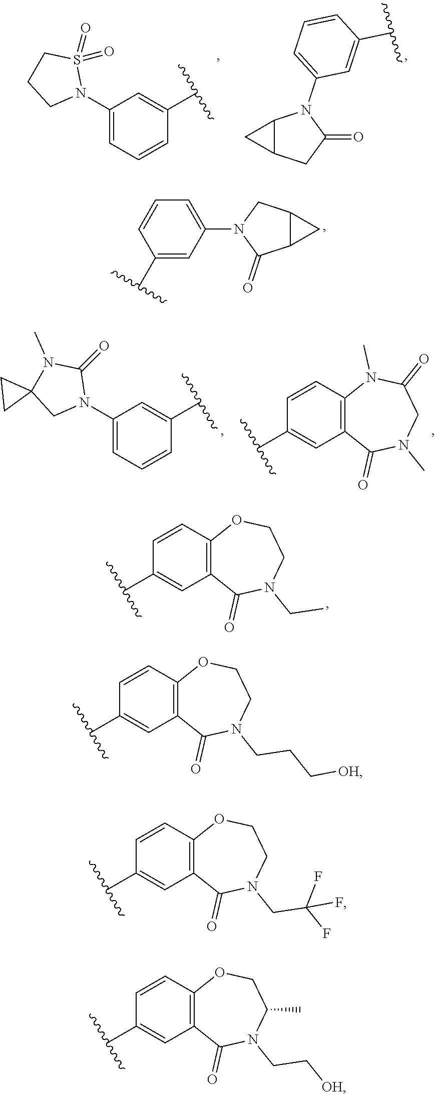

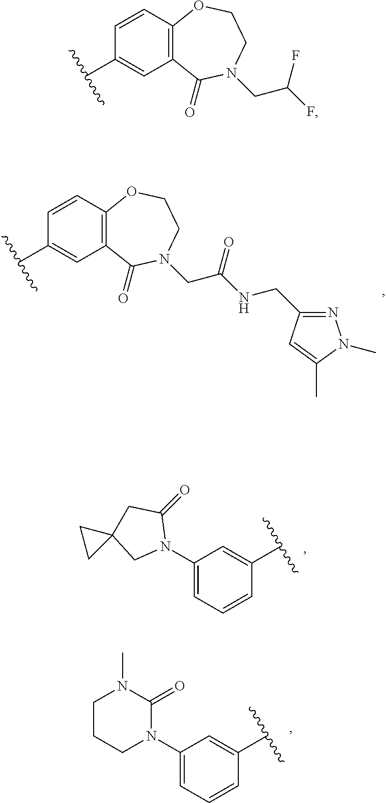 [SMILES: *c1ccc2c(c1)C(=O)N(CC(F)F)CCO2 ; *c1ccc2c(c1)C(=O)N(CC(=O)NCc1cc(C)n(C)n1)CCO2 ; *c1cccc(N2CC3(CC3)CC2=O)c1 ; *c1cccc(N2CCCN(C)C2=O)c1]

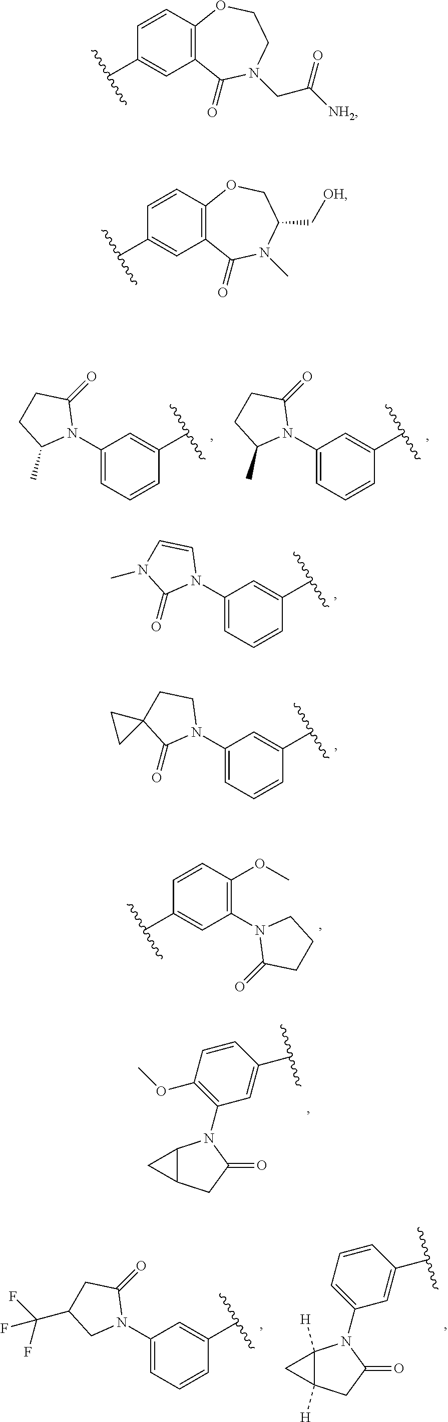

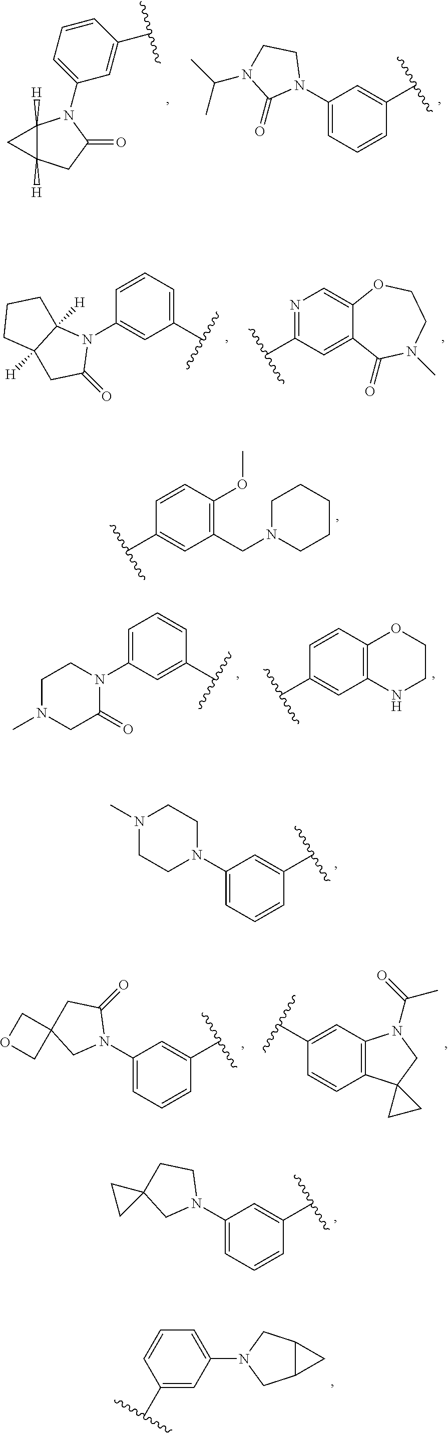

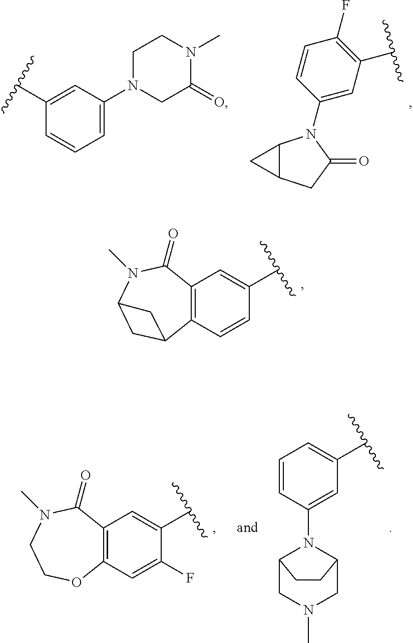 [SMILES: *c1cccc(N2CCN(C)C(=O)C2)c1 ; *c1cc(N2C(=O)CC3CC32)ccc1F ; *c1ccc2c(c1)C(=O)N(C)C1CC2C1 ; *c1cc2c(cc1F)OCCN(C)C2=O ; *c1cccc(N2C3CCC2CN(C)C3)c1]

291. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₁ is selected from:

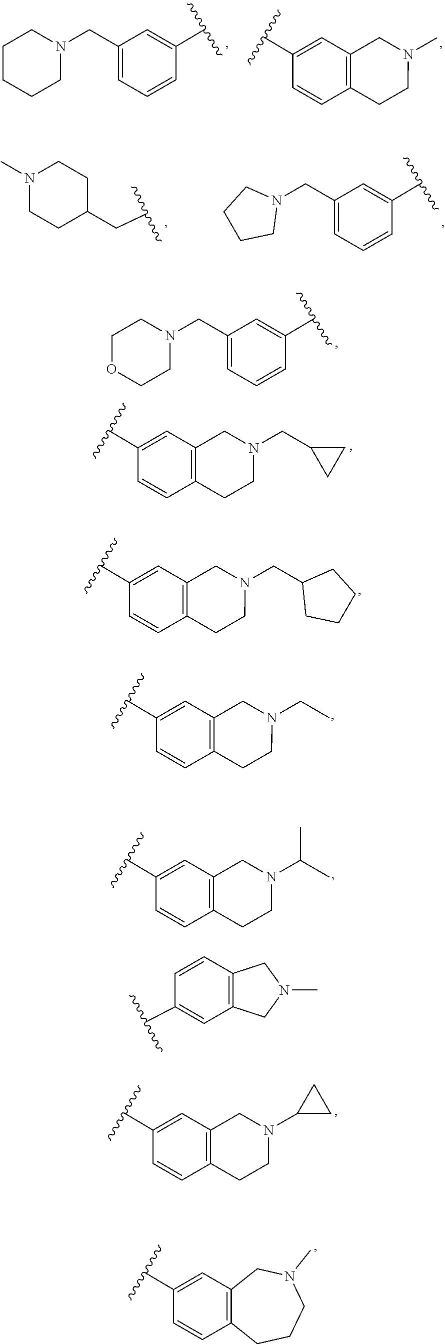

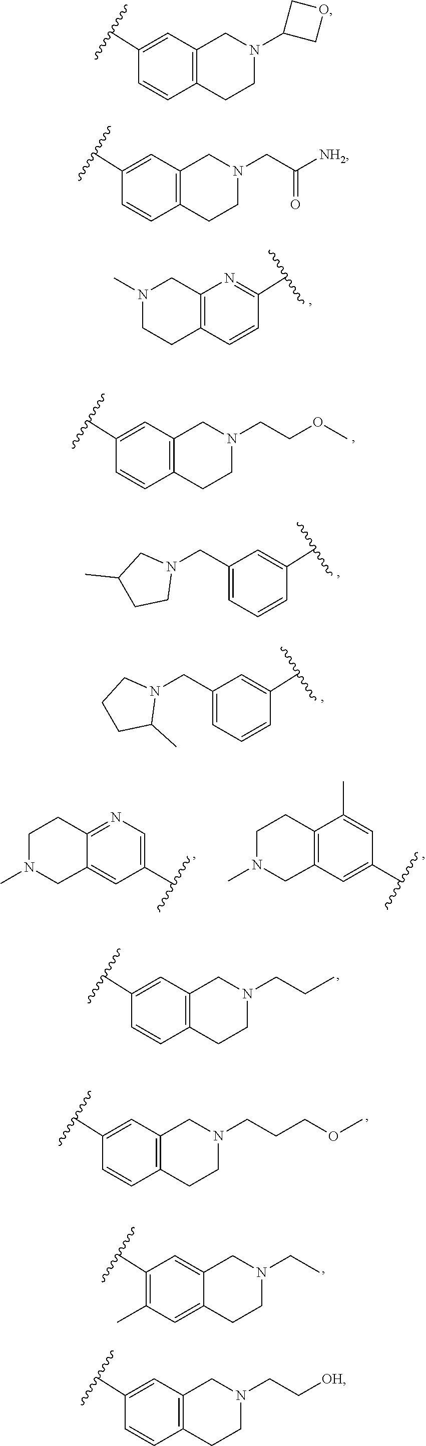

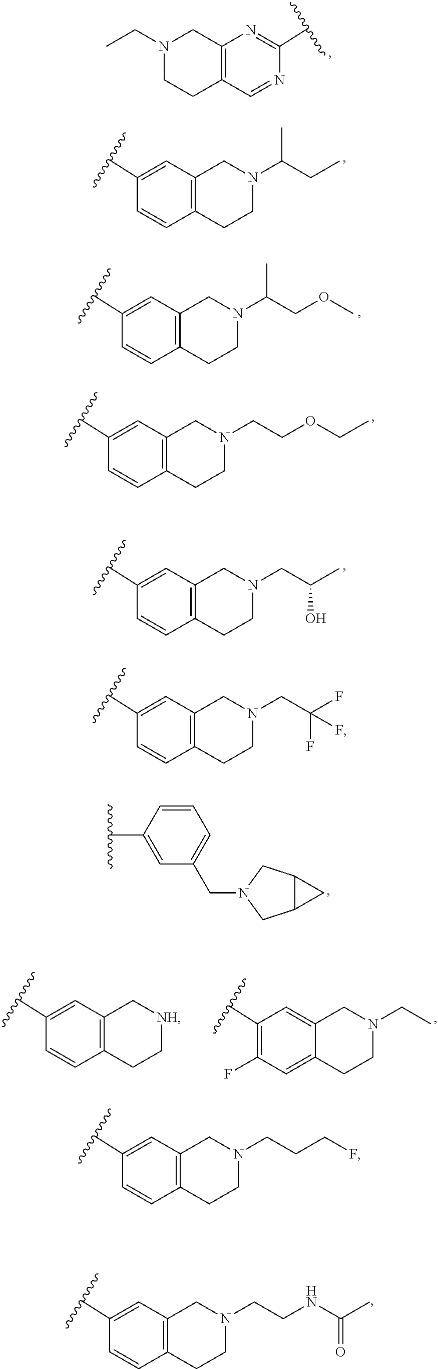

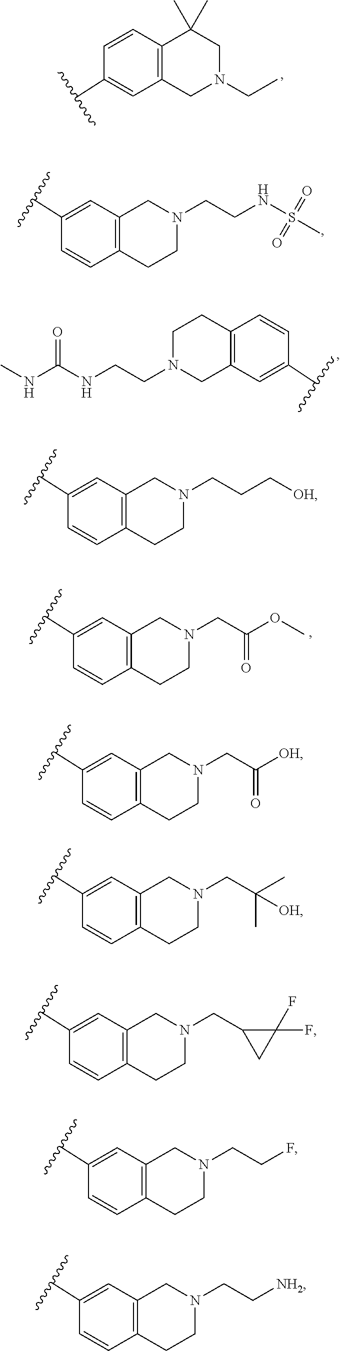

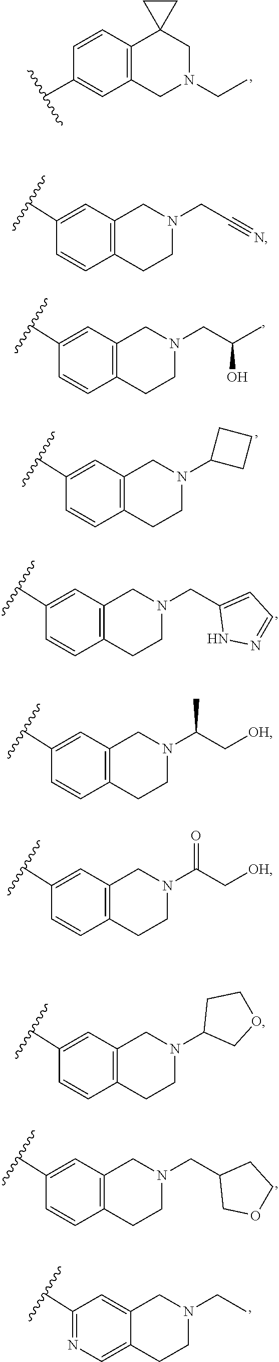

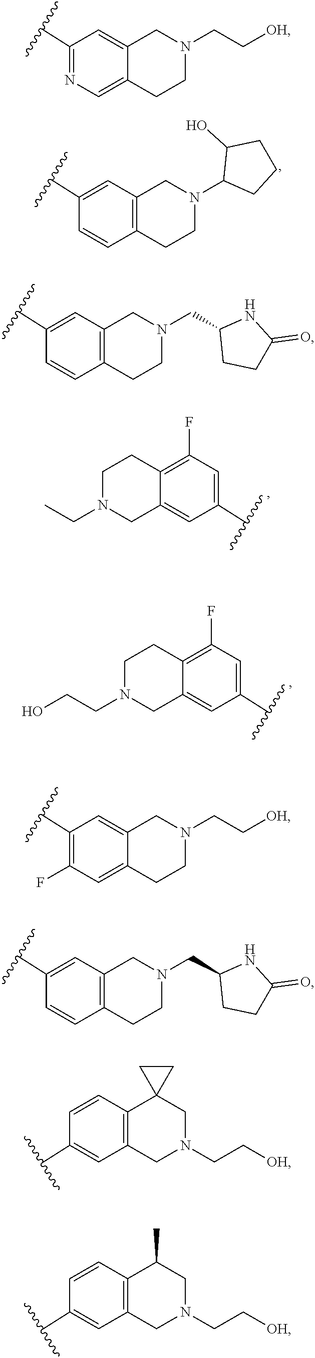

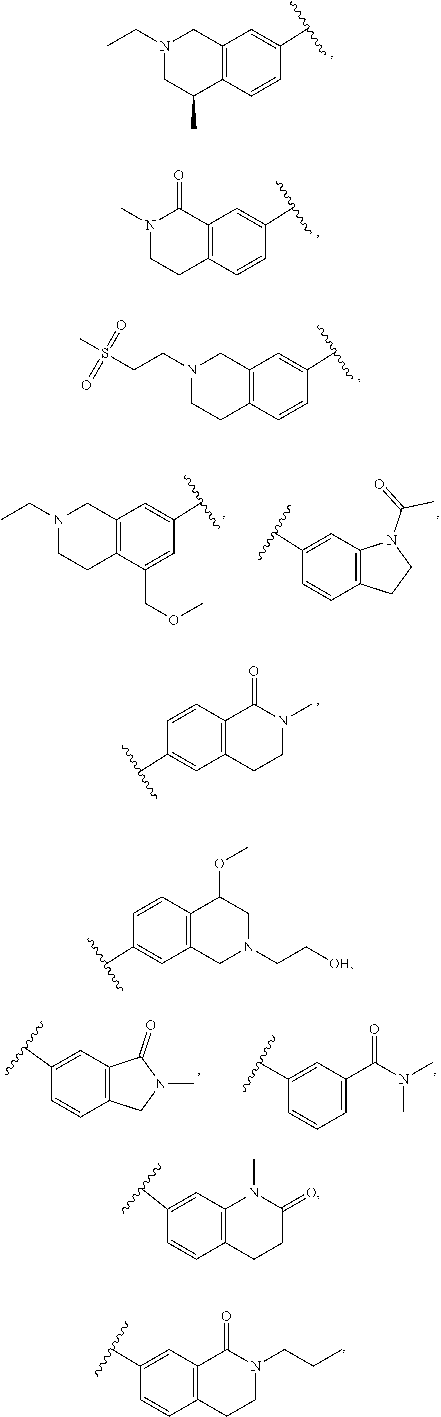

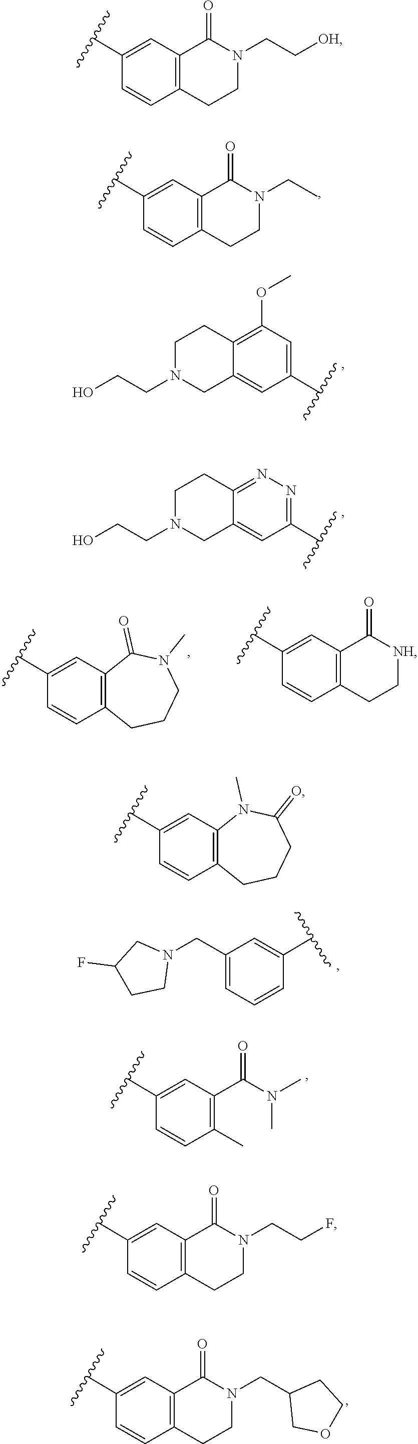

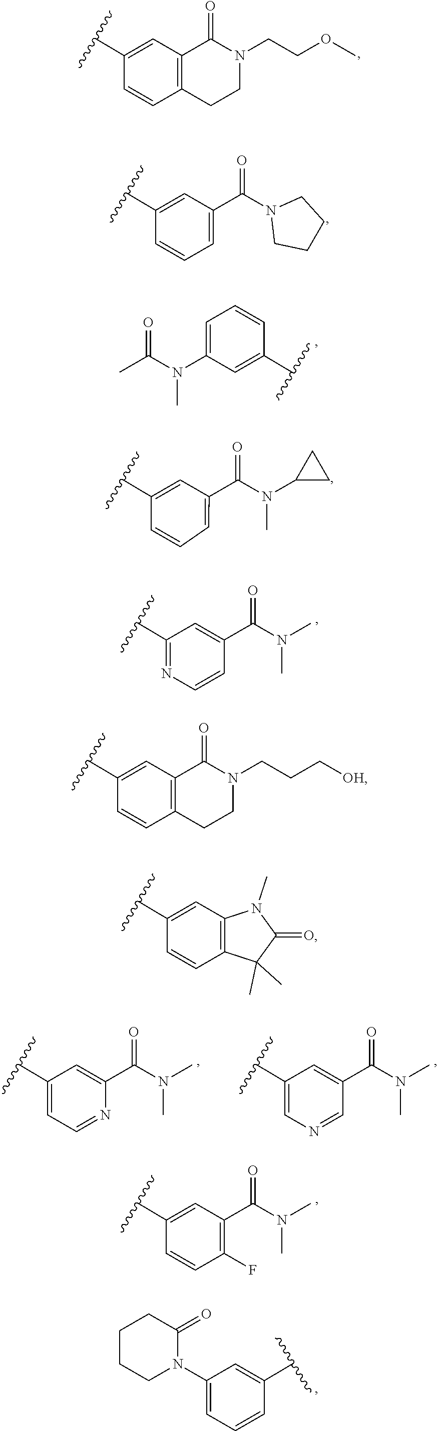

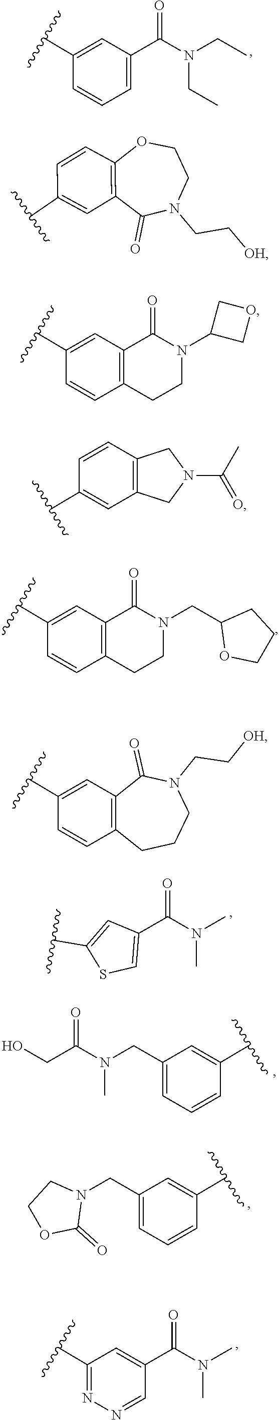

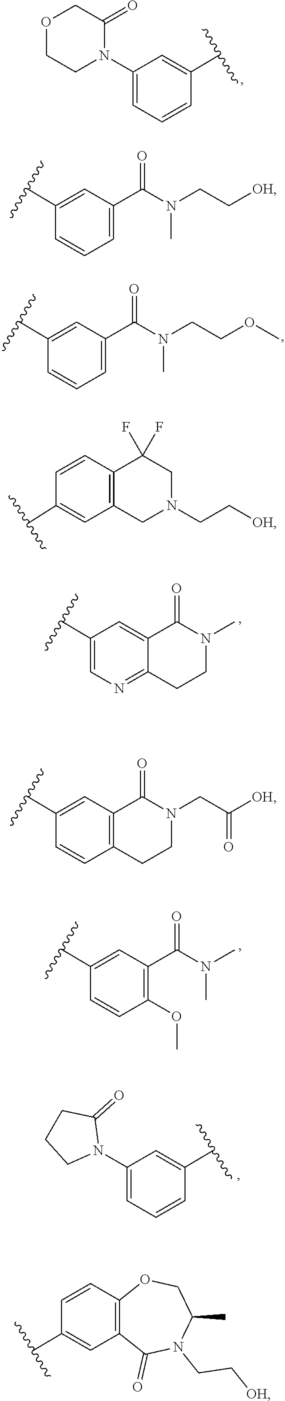

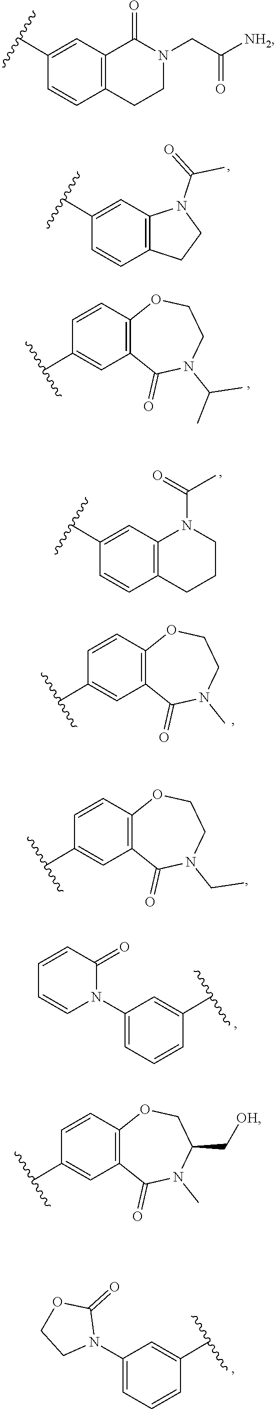

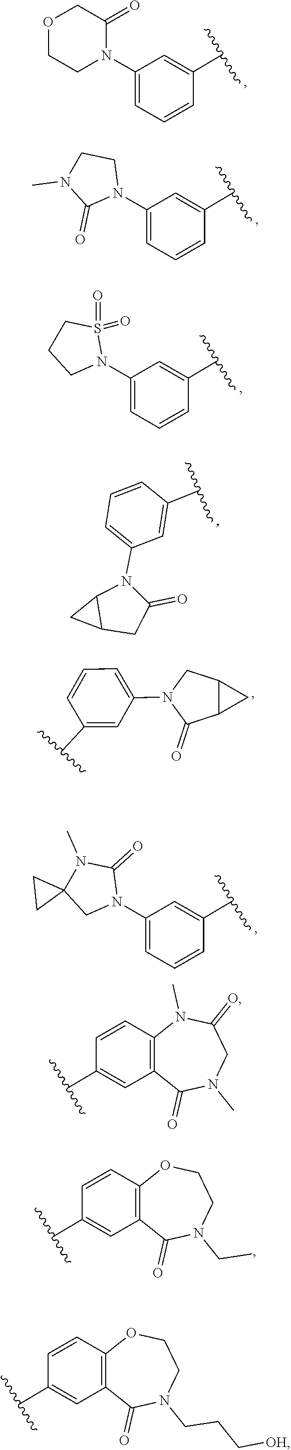

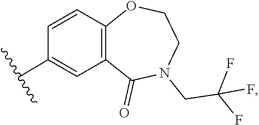 [SMILES: *c1ccc2c(c1)C(=O)N(CC(F)(F)F)CCO2]

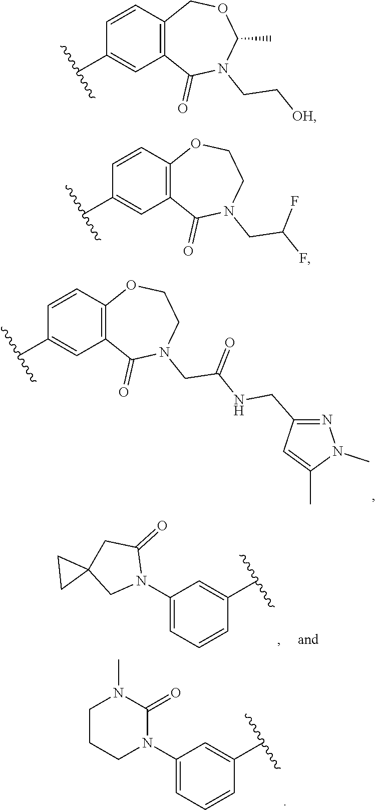

292. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₁ is selected from:

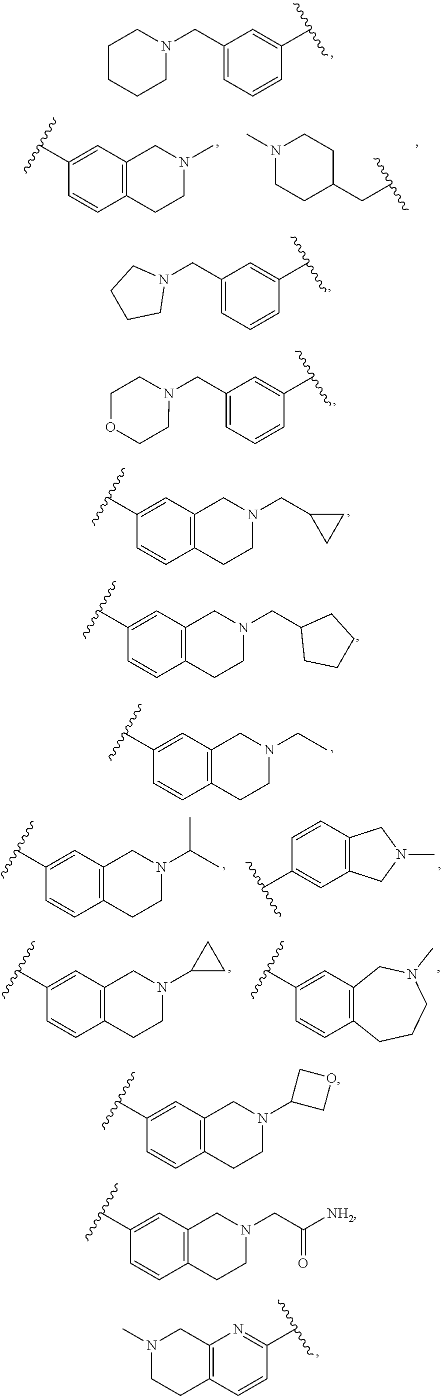

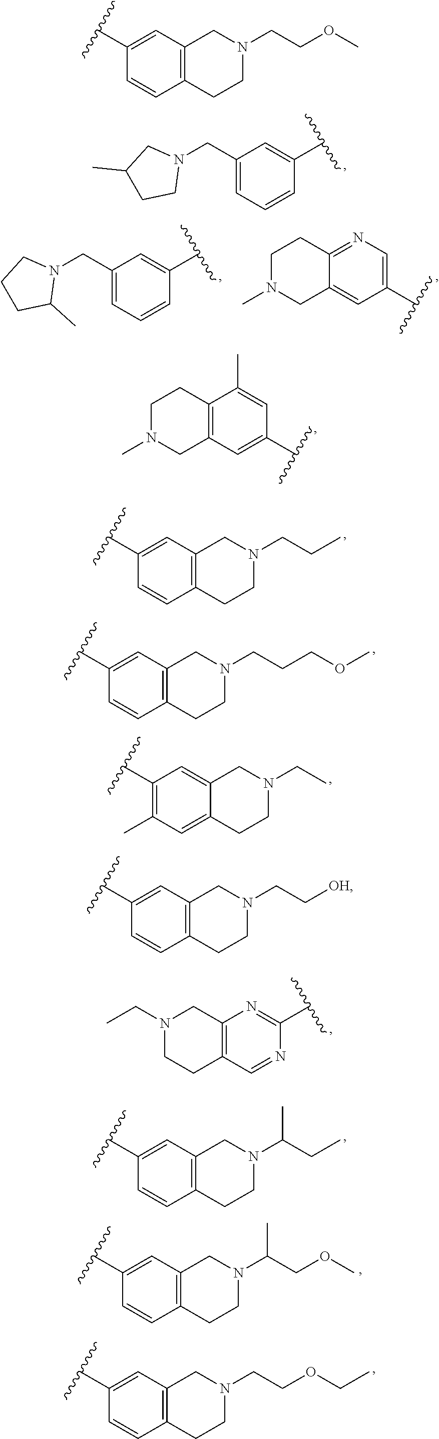

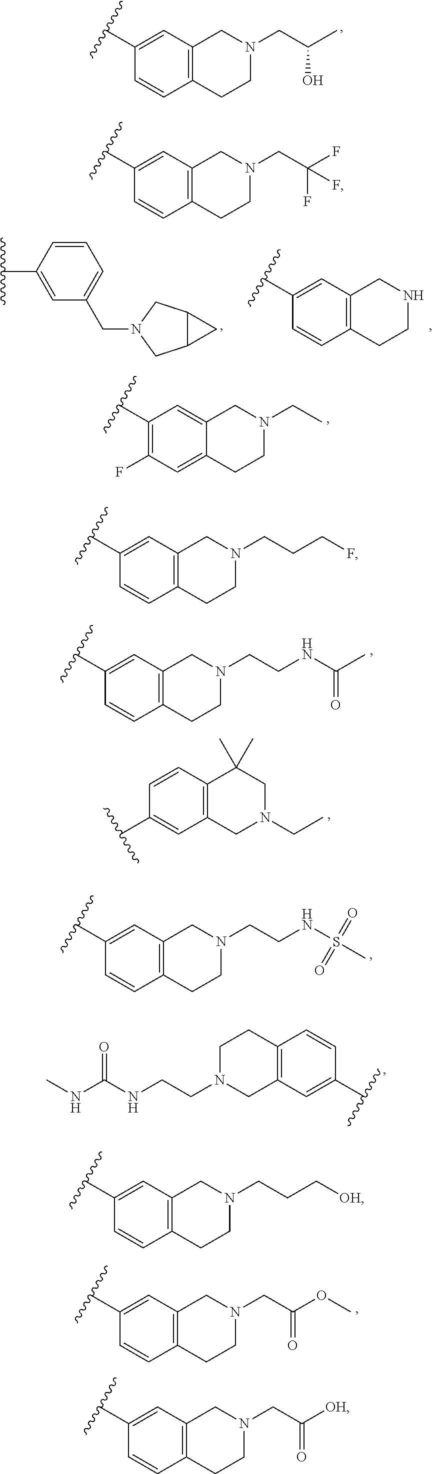

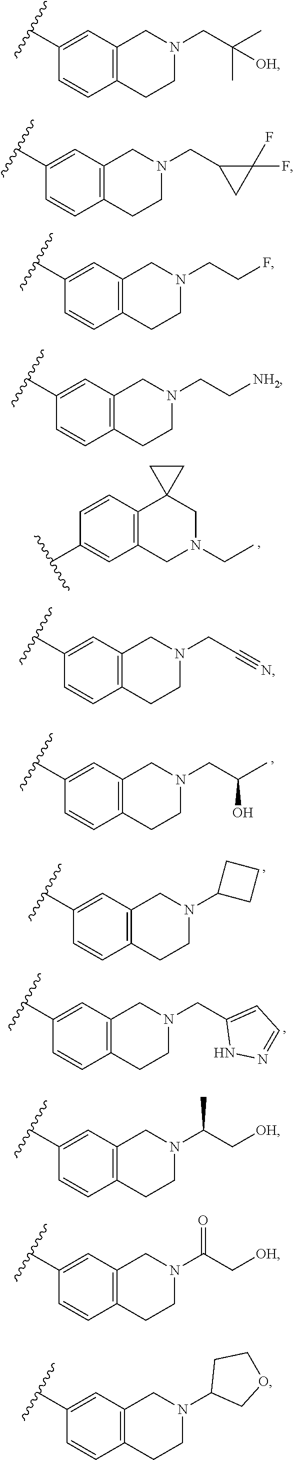

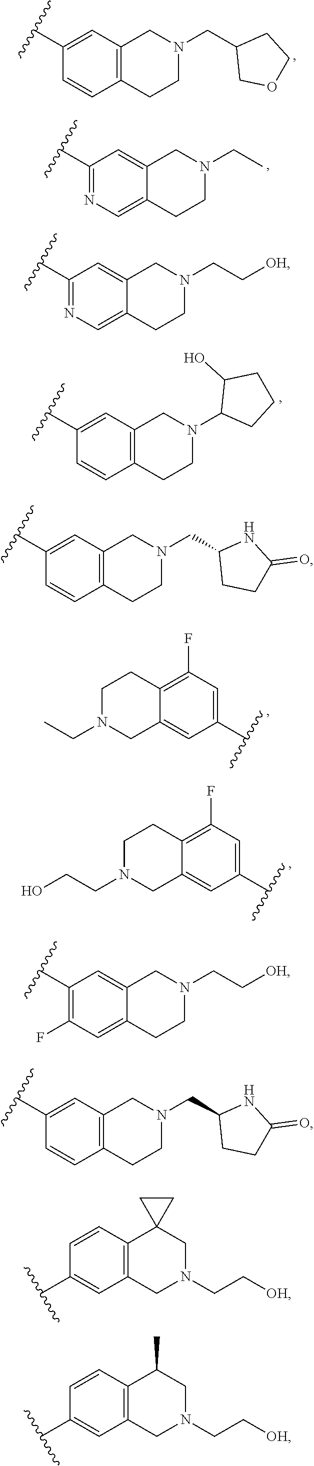

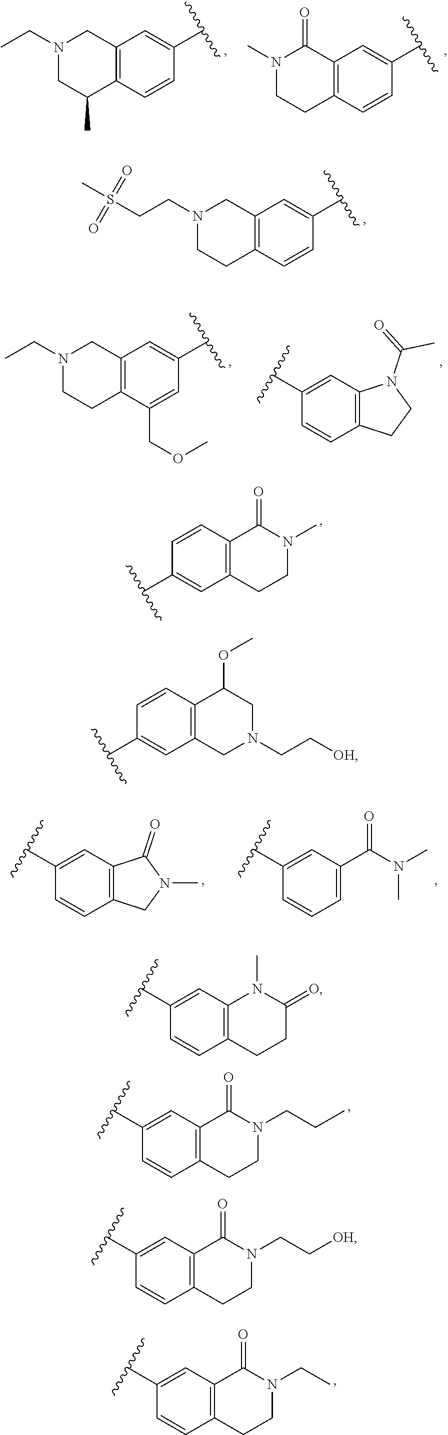

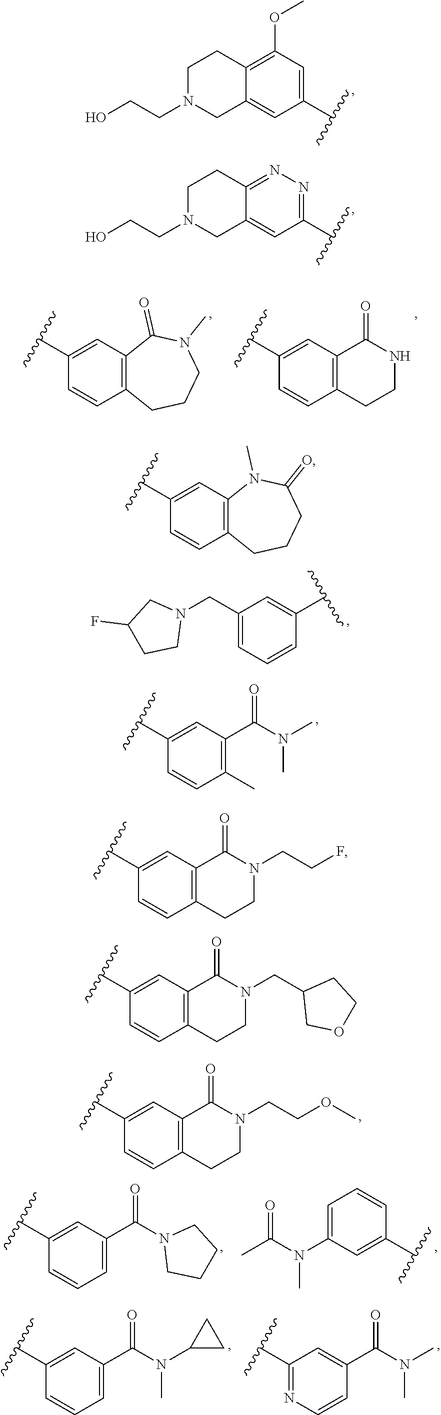

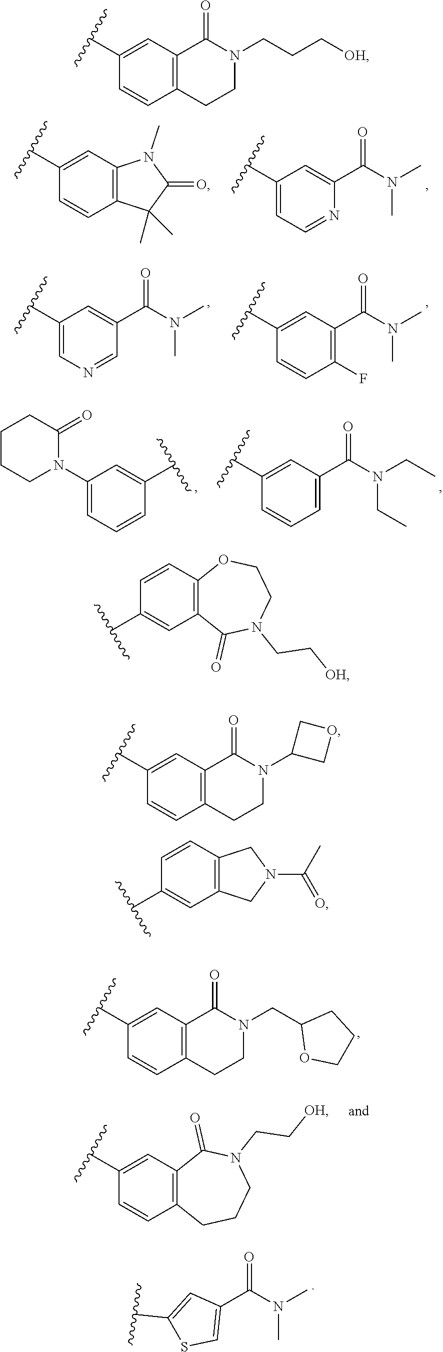

293. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₁ is selected from:

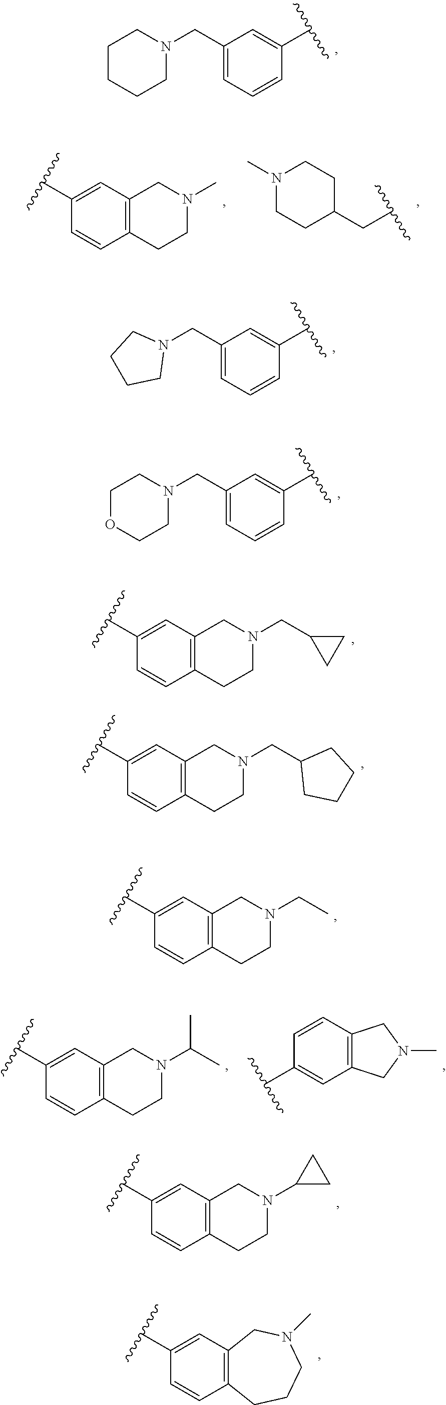

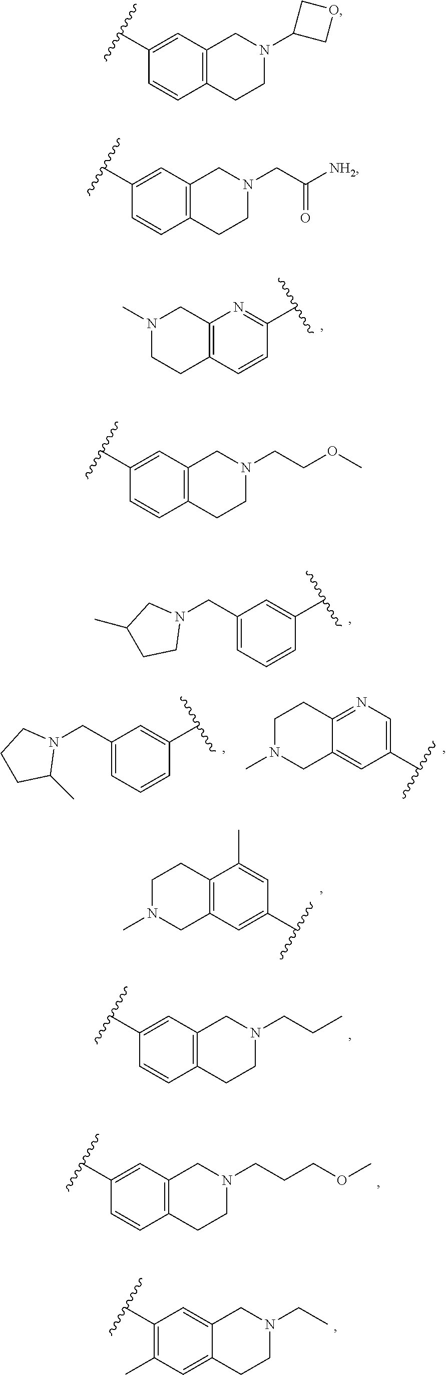

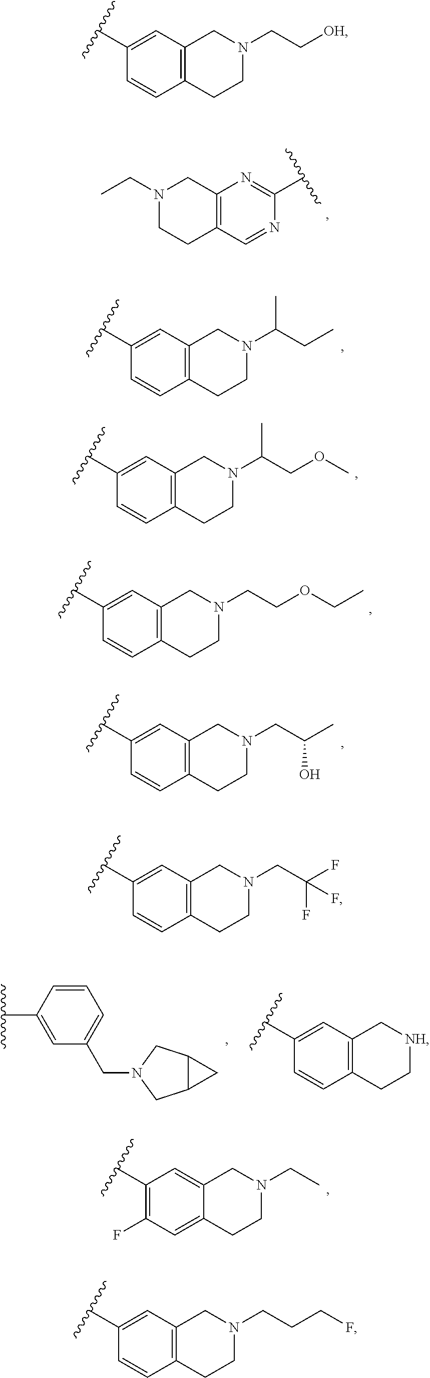

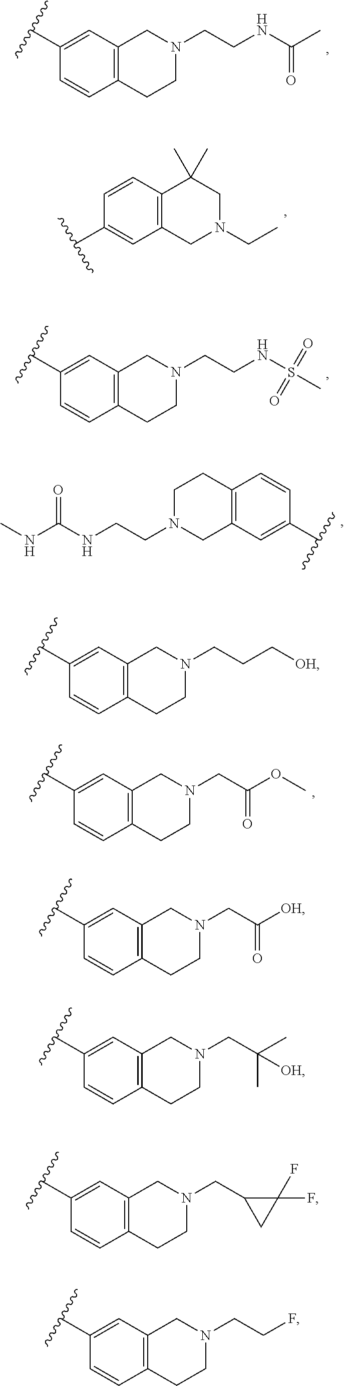

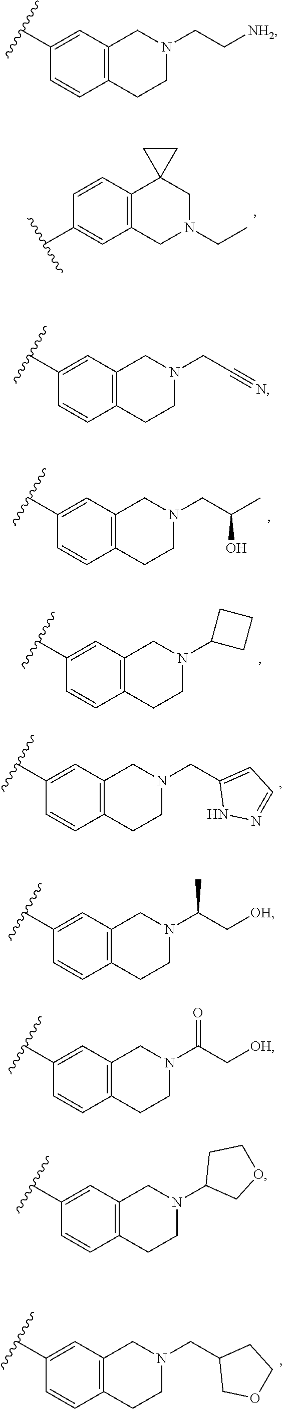

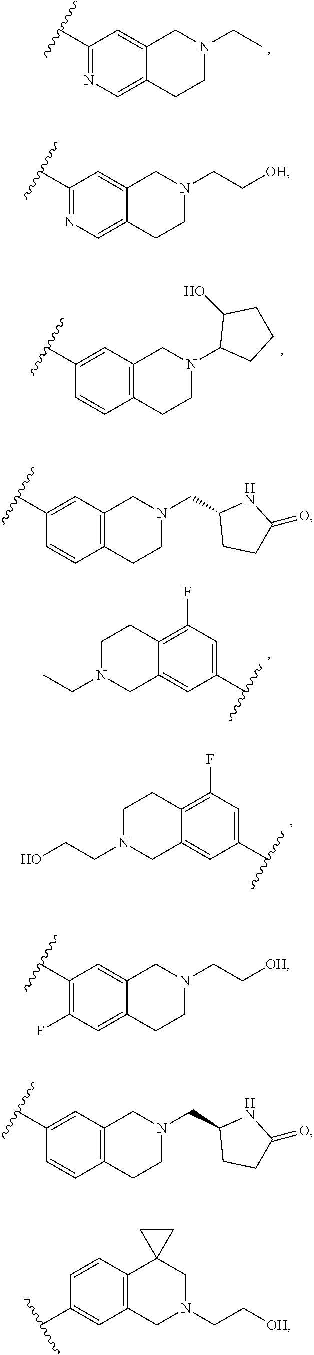

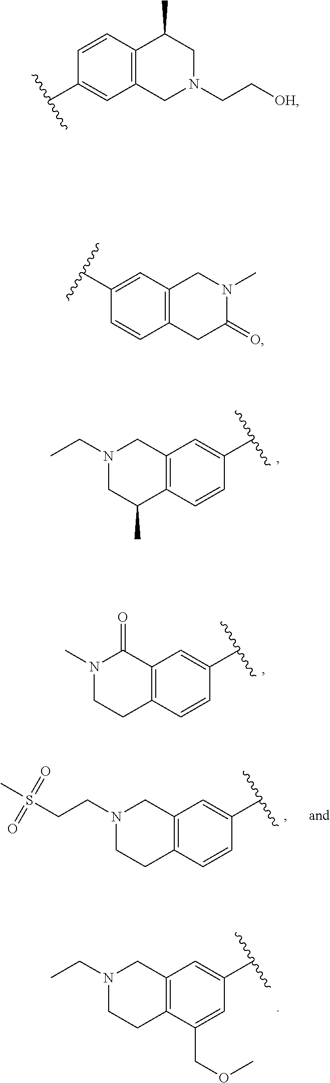 [SMILES: *c1ccc2c(c1)CN(CCO)C[C@@H]2C ; *c1ccc2c(c1)CN(C)C(=O)C2 ; *c1ccc2c(c1)CN(CC)C[C@@H]2C ; *c1ccc2c(c1)C(=O)N(C)CC2 ; *c1ccc2c(c1)CN(CCS(C)(=O)=O)CC2 ; *c1cc(COC)c2c(c1)CN(CC)CC2]

294. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₁ is selected from:

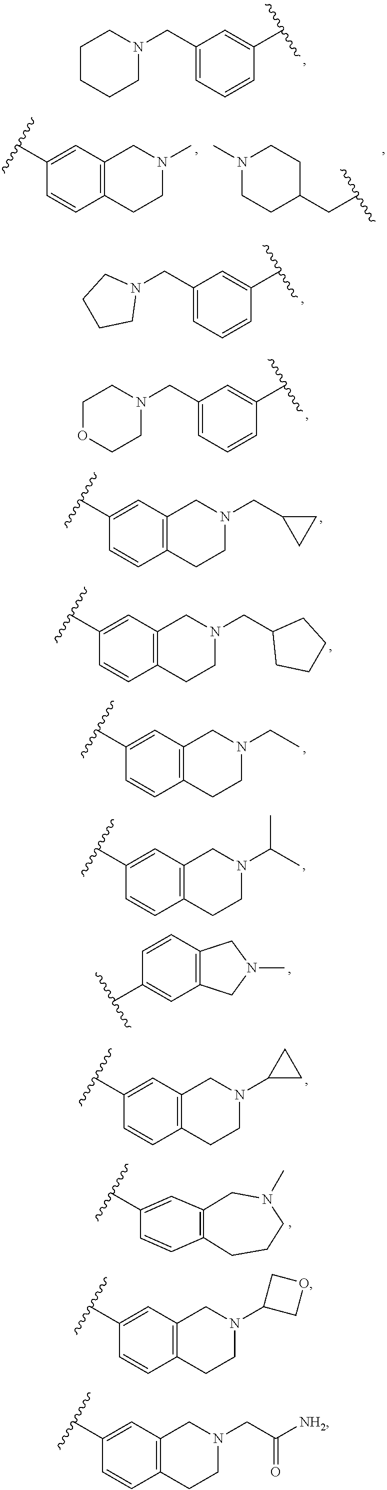

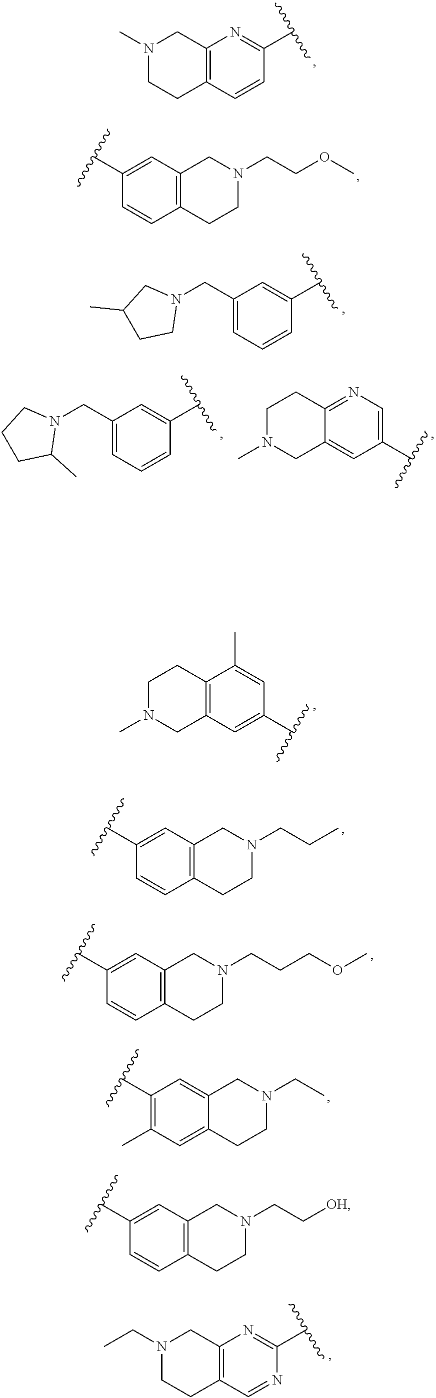

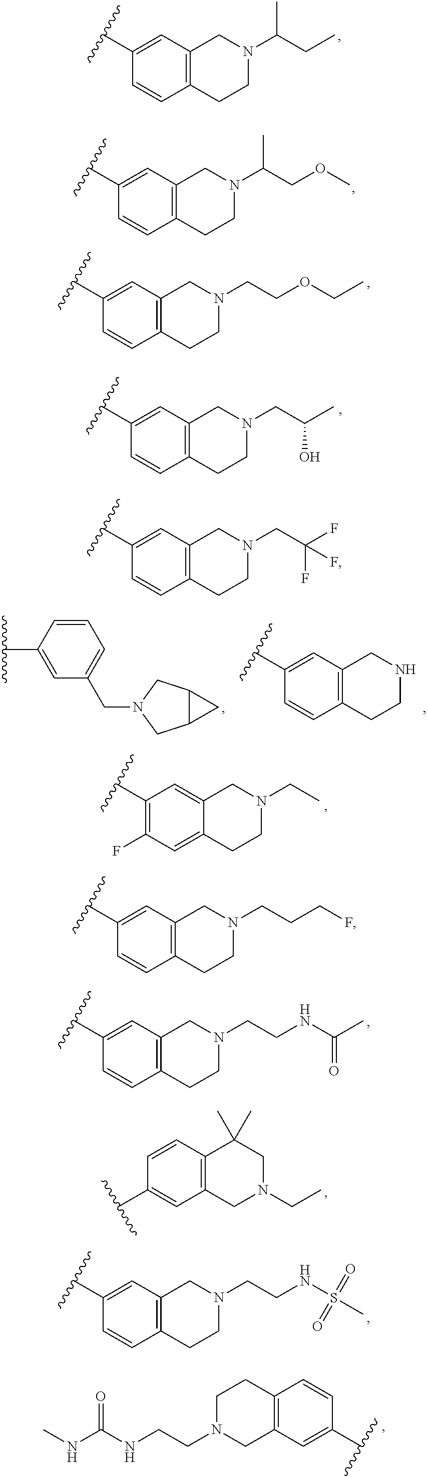

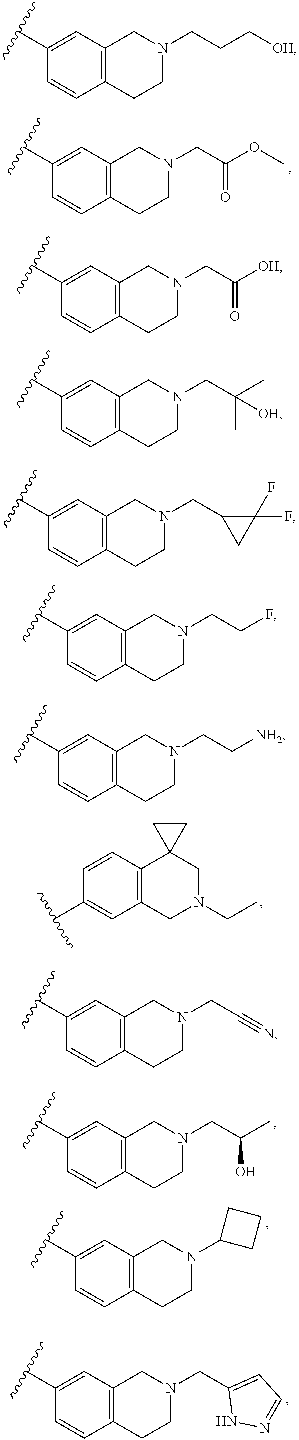

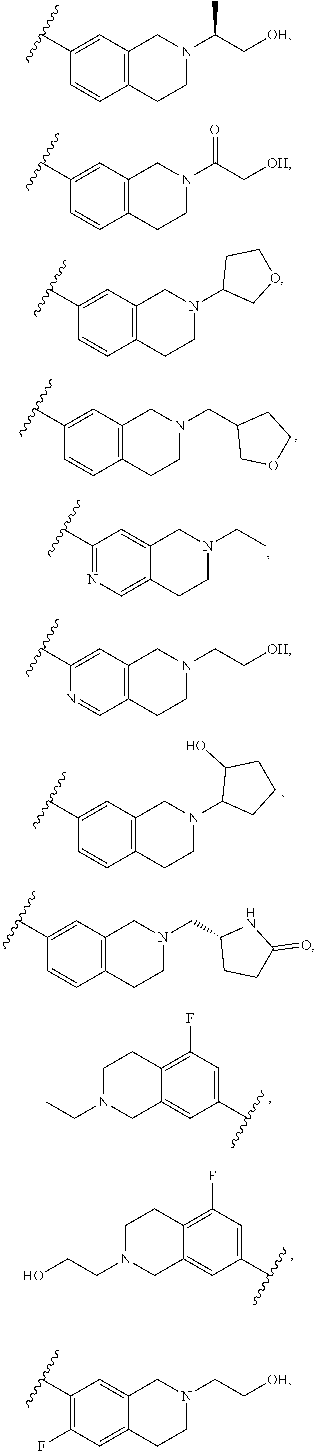

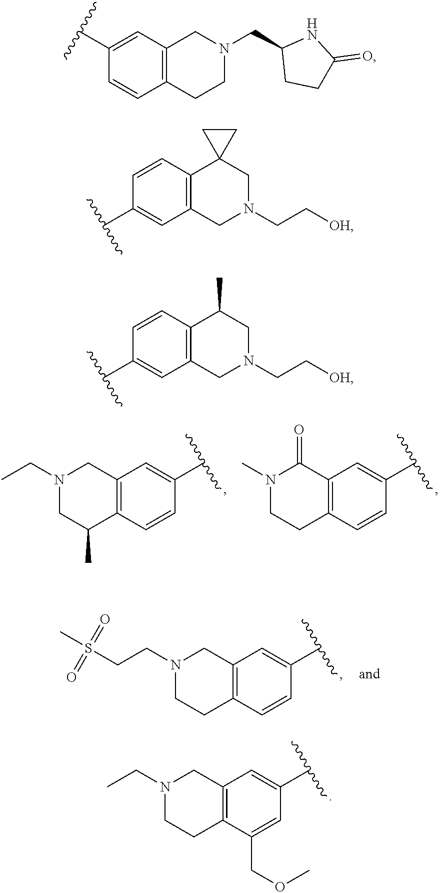

295. A compound according to any of numbered embodiments 1 to 290, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₁ is selected from:

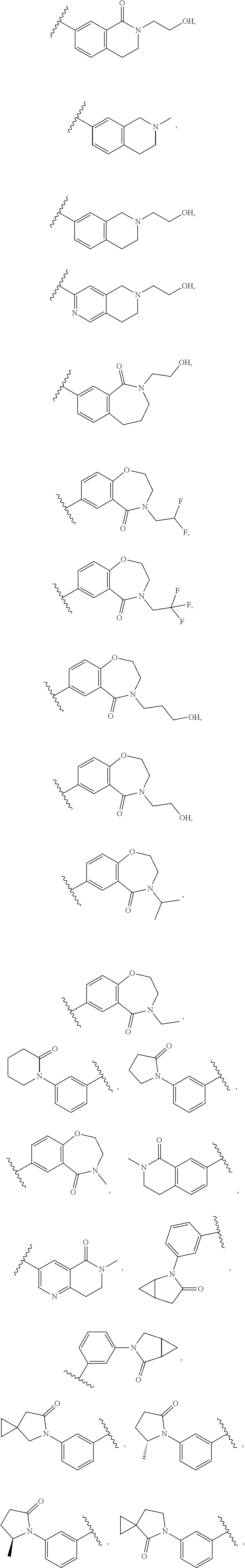

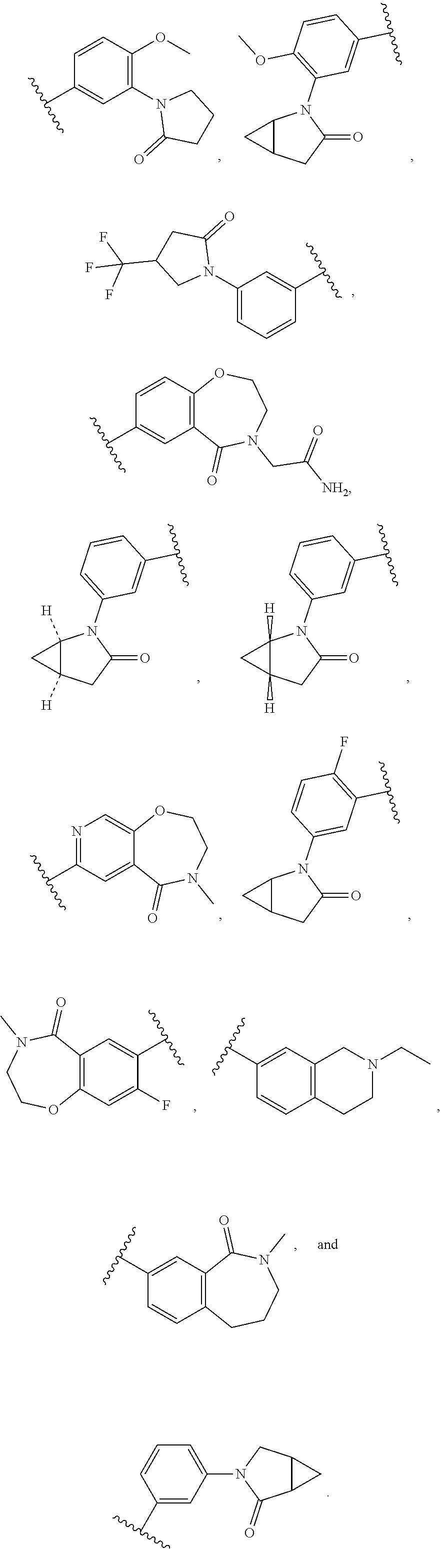

296. A compound according to any of numbered embodiments 1 to 290, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₁ is selected from:

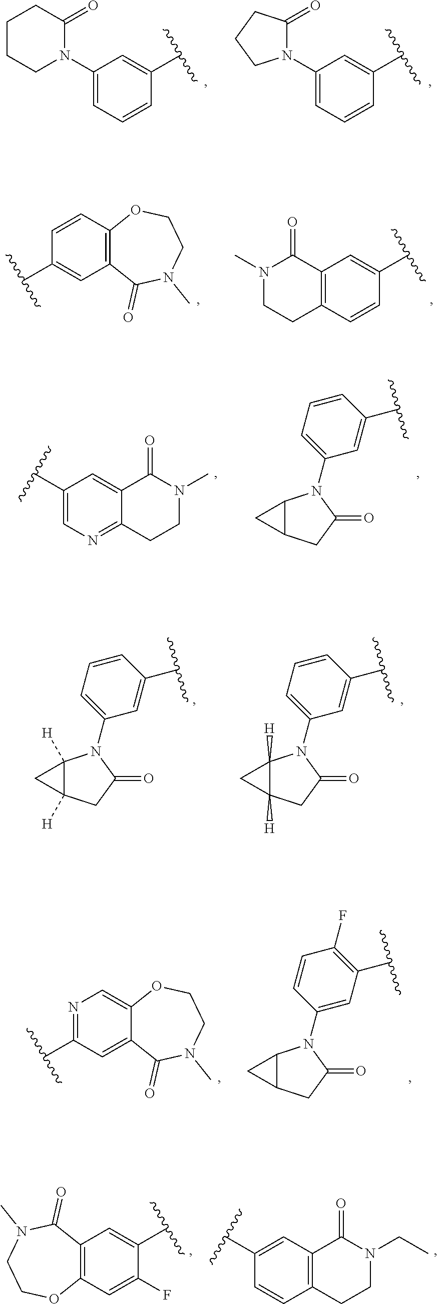

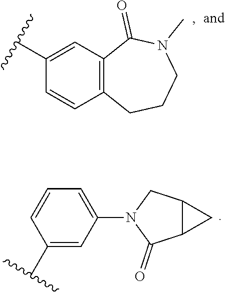 [SMILES: *c1ccc2c(c1)C(=O)N(C)CCC2 ; *c1cccc(N2CC3CC3C2=O)c1]

297. A compound according to any of numbered embodiments 1 to 290, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₁ is selected from

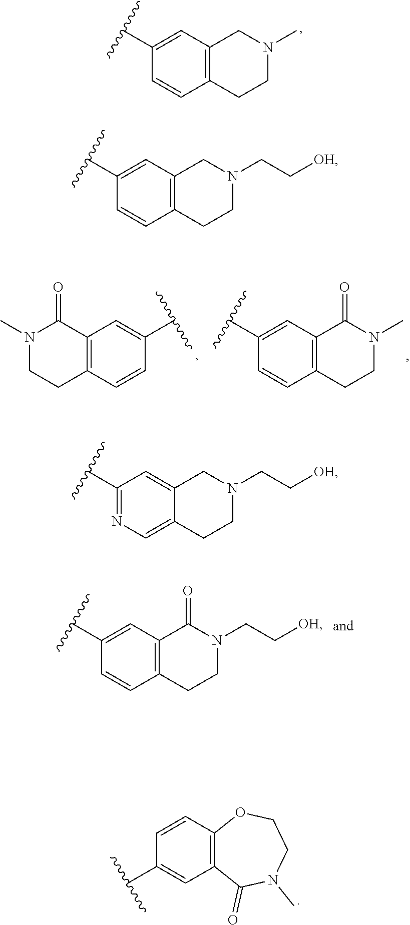

298. A compound according to any of numbered embodiments 1 to 290, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₁ is selected from

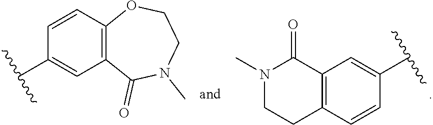 [SMILES: *c1ccc2c(c1)C(=O)N(C)CCO2 ; *c1ccc2c(c1)C(=O)N(C)CC2]

299. A compound according to any of numbered embodiments 1 to 290, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₁ is

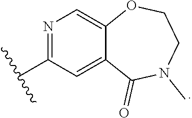 [SMILES: *c1cc2c(cn1)OCCN(C)C2=O]

300. A compound according to any of numbered embodiments 1 to 199, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₁ is

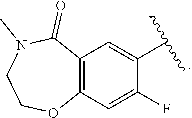 [SMILES: *c1cc2c(cc1F)OCCN(C)C2=O]

301. A compound according to any of numbered embodiments 1 to 291, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₁ is selected from:

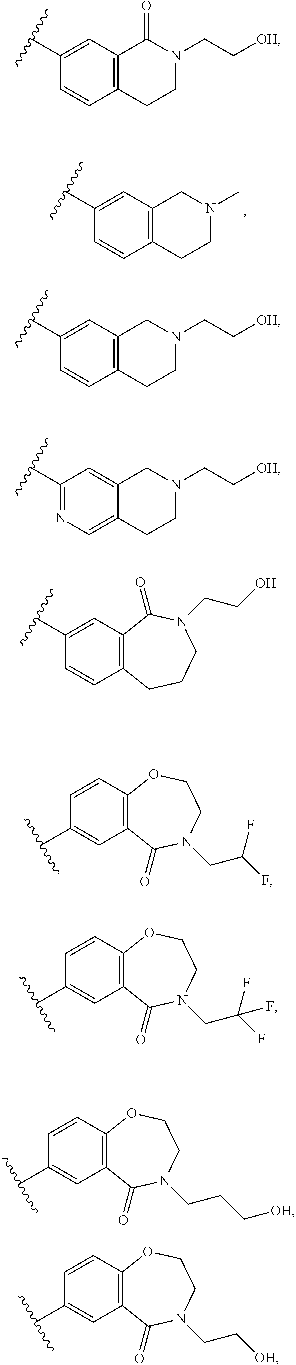

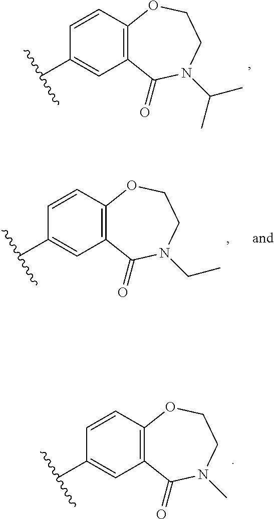 [SMILES: *c1ccc2c(c1)C(=O)N(C(C)C)CCO2 ; *c1ccc2c(c1)C(=O)N(CC)CCO2 ; *c1ccc2c(c1)C(=O)N(C)CCO2]

302. A compound according to any of numbered embodiments 1 to 292, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₁ is selected from:

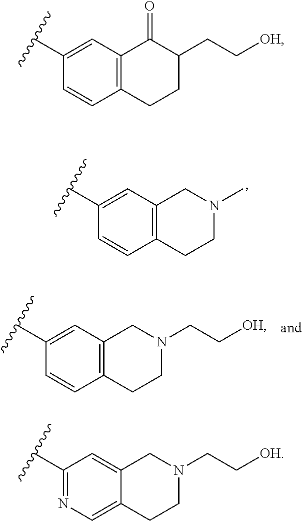 [SMILES: *c1ccc2c(c1)C(=O)C(CCO)CC2 ; *c1ccc2c(c1)CN(C)CC2 ; *c1ccc2c(c1)CN(CCO)CC2 ; *c1cc2c(cn1)CCN(CCO)C2]

303. A compound according to any of numbered embodiments 1 to 291, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₁ is selected from:

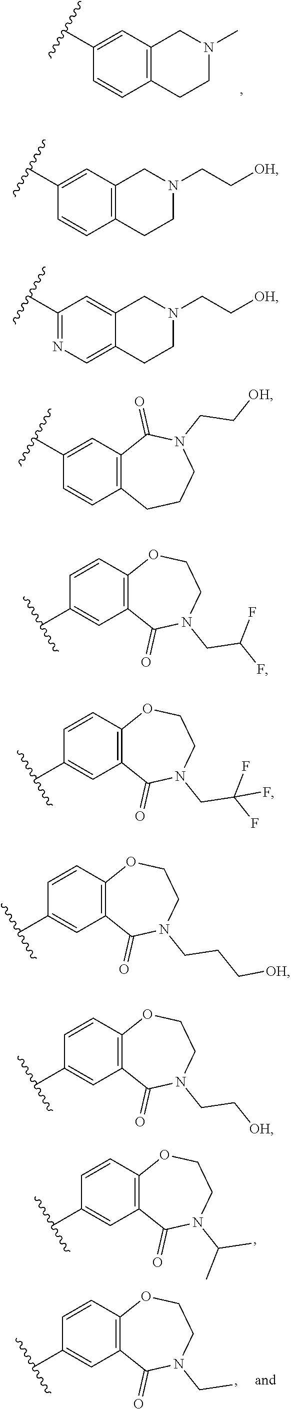

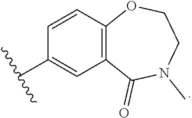 [SMILES: *c1ccc2c(c1)C(=O)N(C)CCO2]

304. A compound according to any of numbered embodiments 1 to 292, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₁ is from: selected

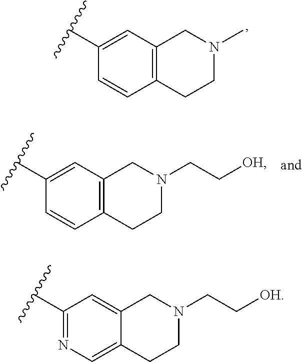 [SMILES: *c1ccc2c(c1)CN(C)CC2 ; *c1ccc2c(c1)CN(CCO)CC2 ; *c1cc2c(cn1)CCN(CCO)C2]

305. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₃ is not

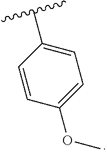 [SMILES: *c1ccc(OC)cc1]

306. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₃ is selected from substituted phenyl, substituted pyridyl, and substituted thiophene.

307. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₃ is substituted phenyl or substituted pyridyl.

308. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₃ is substituted phenyl.

309. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof, wherein, when R₃ is substituted with one substituent only, R₃ is not substituted with a 4-methoxy group.

310. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof, wherein, when R₃ is a substituted six-membered ring, R₃ is not substituted with a methoxy group at the para-position to the attachment to B_{6.}

311. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₃ is not substituted with a 4-methoxy group.

312. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof, wherein, when R₃ is a substituted six-membered ring, R₃ is not substituted with an alkoxy group at the para-position to the attachment to B_{6.}

313. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₃ is not substituted with a 4-alkoxy group.

314. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof, wherein, when R₃ is substituted with three substituents, at least one substituent is halo.

315. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof, wherein, when R₃ is substituted with three substituents, at least one substituent is F or Cl.

316. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₃ is substituted with one or two substituents independently selected from small alkyl^{b}, small alkoxy, cyclopropyl, OH, NH₂, CF₃, halo, —C(═O)—NH₂, —C(═O)—N(H)(small alkyl), and CN.

317. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₃ is substituted with one or two substituents independently selected from small alkyl^{b}, small alkoxy, cyclopropyl, OH, CF₃, halo, —C(═O)—NH₂, —C(═O)—N(H)(small alkyl), and CN.

318. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₃ is substituted with one or two substituents independently selected from small alkyl, small alkoxy, cyclopropyl, OH, CF₃, halo, —C(═O)—NH₂, —C(═O)—N(H)(small alkyl), and CN.

319. A compound according to any of numbered embodiments 1-315, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₃ is substituted with one or two substituents independently selected from small alkyl^{c}, small alkoxy, cyclopropyl, OH, NH₂, CF₃, halo, —C(═O)—NH₂, —C(═O)—N(H)(small alkyl), and CN, wherein small alkyl^{c} is a linear saturated hydrocarbon having up to 4 carbon atoms (C₁-C₄) or a branched saturated hydrocarbon of between 3 and 4 carbon atoms (C₃-C₄); small alkyl^{c} may optionally be substituted with 1 or 2 substituents independently selected from cyclopropyl, OH, OMe, OEt, CN, CF₃, —C(═O)NH₂, and halo.

320. A compound according to any of numbered embodiments 1-315, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₃ is substituted with one or two substituents independently selected from small alkyl^{a}, small alkoxy, cyclopropyl, OH, CF₃, halo, —C(═O)—NH₂, —C(═O)—N(H)(small alkyl), and CN, wherein small alkyl^{a} is a linear saturated hydrocarbon having up to 4 carbon atoms (C₁-C₄) or a branched saturated hydrocarbon of between 3 and 4 carbon atoms (C₃-C₄); small alkyl^{a} may optionally be substituted with 1 or 2 substituents independently selected from cyclopropyl, OH, CN, CF₃, —C(═O)NH₂, and halo.

321. A compound according to any of numbered embodiments 1-315, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₃ is substituted with one or two substituents independently selected from small alkyl^{b}, small alkoxy, cyclopropyl, OH, NH₂, CF₃, halo, —C(═O)—NH₂, —C(═O)—N(H)(small alkyl), —C(═O)—N(small alkyl)(small alkyl), and CN, wherein R₃ is not substituted with any methoxy (—OCH₃) groups.

322. A compound according to any of numbered embodiments 1-315, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₃ is substituted with one or two substituents independently selected from small alkyl, small alkoxy, cyclopropyl, OH, CF₃, halo, —C(═O)—NH₂, —C(═O)—N(H)(small alkyl), —C(═O)—N(small alkyl)(small alkyl), and CN, wherein R₃ is not substituted with any methoxy (—OCH₃) groups.

323. A compound according to any of numbered embodiments 1-315, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₃ is substituted with one or two substituents independently selected from small alkyl^{c}, small alkoxy, cyclopropyl, OH, NH₂, CF₃, halo, —C(═O)—NH₂, —C(═O)—N(H)(small alkyl), —C(═O)—N(small alkyl)(small alkyl), and CN, wherein small alkyl^{c} is a linear saturated hydrocarbon having up to 4 carbon atoms (C₁-C₄) or a branched saturated hydrocarbon of between 3 and 4 carbon atoms (C₃-C₄); small alkyl^{c} may optionally be substituted with 1 or 2 substituents independently selected from cyclopropyl, OH, OMe, OEt, CN, CF₃, —C(═O)NH₂, and halo, and wherein R₃ is not substituted with any methoxy (—OCH₃) groups.

324. A compound according to any of numbered embodiments 1-315, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₃ is substituted with one or two substituents independently selected from small alkyl^{c}, small alkoxy, cyclopropyl, OH, CF₃, halo, —C(═O)—NH₂, —C(═O)—N(H)(small alkyl), —C(═O)—N(small alkyl)(small alkyl), and CN, wherein small alkyl^{c} is a linear saturated hydrocarbon having up to 4 carbon atoms (C₁-C₄) or a branched saturated hydrocarbon of between 3 and 4 carbon atoms (C₃-C₄); small alkyl^{c} may optionally be substituted with 1 or 2 substituents independently selected from cyclopropyl, OH, OMe, OEt, CN, CF₃, —C(═O)NH₂, and halo, and wherein R₃ is not substituted with any methoxy (—OCH₃) groups.

325. A compound according to any of numbered embodiments 1-315, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₃ is substituted with one or two substituents independently selected from small alkyl^{a}, small alkoxy, cyclopropyl, OH, CF₃, halo, —C(═O)—NH₂, —C(═O)—N(H)(small alkyl), —C(═O)—N(small alkyl)(small alkyl), and CN, wherein small alkyl^{a} is a linear saturated hydrocarbon having up to 4 carbon atoms (C₁-C₄) or a branched saturated hydrocarbon of between 3 and 4 carbon atoms (C₃-C₄); small alkyl^{a} may optionally be substituted with 1 or 2 substituents independently selected from cyclopropyl, OH, CN, CF₃, —C(═O)NH₂, and halo, and wherein R₃ is not substituted with any methoxy (—OCH₃) groups.

326. A compound according to numbered embodiment 321, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₃ is substituted with one or two substituents independently selected from small alkyl^{b}, cyclopropyl, OH, NH₂, CF₃, halo, —C(═O)—NH₂, —C(═O)—N(H)(small alkyl), —C(═O)—N(small alkyl)(small alkyl), and CN.

327. A compound according to numbered embodiment 322, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₃ is substituted with one or two substituents independently selected from small alkyl, cyclopropyl, OH, CF₃, halo, —C(═O)—NH₂, —C(═O)—N(H)(small alkyl), —C(═O)—N(small alkyl)(small alkyl), and CN.

328. A compound according to numbered embodiment 323, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₃ is substituted with one or two substituents independently selected from small alkyl^{c}, cyclopropyl, OH, NH₂, CF₃, halo, —C(═O)—NH₂, —C(═O)—N(H)(small alkyl), —C(═O)—N(small alkyl)(small alkyl), and CN, wherein small alkyl^{c} is a linear saturated hydrocarbon having up to 4 carbon atoms (C₁-C₄) or a branched saturated hydrocarbon of between 3 and 4 carbon atoms (C₃-C₄); small alkyl^{c} may optionally be substituted with 1 or 2 substituents independently selected from cyclopropyl, OH, OMe, OEt, CN, CF₃, —C(═O)NH₂, and halo.

329. A compound according to numbered embodiment 325, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₃ is substituted with one or two substituents independently selected from small alkyl^{a}, cyclopropyl, OH, CF₃, halo, —C(═O)—NH₂, —C(═O)—N(H)(small alkyl), —C(═O)—N(small alkyl)(small alkyl), and CN, wherein small alkyl^{a} is a linear saturated hydrocarbon having up to 4 carbon atoms (C₁-C₄) or a branched saturated hydrocarbon of between 3 and 4 carbon atoms (C₃-C₄); small alkyl^{a} may optionally be substituted with 1 or 2 substituents independently selected from cyclopropyl, OH, CN, CF₃, —C(═O)NH₂, and halo.

330. A compound according to any of numbered embodiments 1-315, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₃ is substituted with one or two substituents independently selected from small alkyl^{b}, small alkoxy, OH, NH₂, CF₃, halo, —C(═O)—NH₂, and CN.

331. A compound according to any of numbered embodiments 1-315, or a tautomer, isomer, wherein R₃ is substituted with one or two substituents independently selected from small alkyl, small alkoxy, CF₃, halo, —C(═O)—NH₂, and CN.

332. A compound according to any of numbered embodiments 1-315, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₃ is substituted with one or two substituents independently selected from small alkyl^{c}, small alkoxy, OH, NH₂, CF₃, halo, —C(═O)—NH₂, and CN, wherein small alkyl^{c} is a linear saturated hydrocarbon having up to 4 carbon atoms (C₁-C₄) or a branched saturated hydrocarbon of between 3 and 4 carbon atoms (C₃-C₄); small alkyl^{c} may optionally be substituted with 1 or 2 substituents independently selected from cyclopropyl, OH, OMe, OEt, CN, CF₃, —C(═O)NH₂, and halo.

333. A compound according to any of numbered embodiments 1-315, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₃ is substituted with one or two substituents independently selected from small alkyl^{a}, small alkoxy, CF₃, halo, —C(═O)—NH₂, and CN, wherein small alkyl^{a} is a linear saturated hydrocarbon having up to 4 carbon atoms (C₁-C₄) or a branched saturated hydrocarbon of between 3 and 4 carbon atoms (C₃-C₄); small alkyl^{a} may optionally be substituted with 1 or 2 substituents independently selected from cyclopropyl, OH, CN, CF₃, —C(═O)NH₂, and halo.

334. A compound according to numbered embodiment 330, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₃ is substituted with one or two substituents independently selected from small alkyl^{b}, small alkoxy, OH, NH₂, CF₃, halo, —C(═O)—NH₂, and CN, wherein R₃ is not substituted with a 4-methoxy (—OCH₃) group.

335. A compound according to numbered embodiment 331, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₃ is substituted with one or two substituents independently selected from small alkyl, small alkoxy, CF₃, halo, —C(═O)—NH₂, and CN, wherein R₃ is not substituted with a 4-methoxy (—OCH₃) group.

336. A compound according to numbered embodiment 332, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₃ is substituted with one or two substituents independently selected from small alkyl^{c}, small alkoxy, OH, NH₂, CF₃, halo, —C(═O)—NH₂, and CN, wherein small alkyl^{c} is a linear saturated hydrocarbon having up to 4 carbon atoms (C₁-CA) or a branched saturated hydrocarbon of between 3 and 4 carbon atoms (C₃-C₄); small alkyl^{c} may optionally be substituted with 1 or 2 substituents independently selected from cyclopropyl, OH, OMe, OEt, CN, CF₃, —C(═O)NH₂, and halo, and wherein R₃ is not substituted with a 4-methoxy (—OCH₃) group.

337. A compound according to numbered embodiment 333, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₃ is substituted with one or two substituents independently selected from small alkyl^{a}, small alkoxy, CF₃, halo, —C(═O)—NH₂, and CN, wherein small alkyl^{a} is a linear saturated hydrocarbon having up to 4 carbon atoms (C₁-C₄) or a branched saturated hydrocarbon of between 3 and 4 carbon atoms (C₃-C₄); small alkyl^{a} may optionally be substituted with 1 or 2 substituents independently selected from cyclopropyl, OH, CN, CF₃, —C(═O)NH₂, and halo, and wherein R₃ is not substituted with a 4-methoxy (—OCH₃) group.

338. A compound according to any preceding numbered embodiment, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₃ is substituted with one or two substituents independently selected from -CH₃, —CH₂—OH, —CH₂—NH₂, —CH₂—O—CH₃,-OCH₃, OH, NH₂, CF₃, halo, —C(═O)—NH₂, and CN.

339. A compound according to any preceding numbered embodiment, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₃ is substituted with one or two substituents independently selected from -CH₃, —CH₂—OH, —CH₂—NH₂, —CH₂—O—CH₃,-OCH₃, OH, CF₃, halo, —C(═O)—NH₂, and CN.

340. A compound according to any preceding numbered embodiment, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₃ is substituted with one or two substituents independently selected from —CH₃, —CH₂—NH₂, —OCH₃, CF₃, halo, —C(═O)—NH₂, and CN.

341. A compound according to any preceding numbered embodiment, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₃ is substituted with one or two substituents independently selected from —CH₃, —CH₂—OH, —CH₂—O—CH₃, —OCH₃, OH, NH₂, CF₃, halo, —C(═O)—NH₂, and CN.

342. A compound according to any preceding numbered embodiment, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₃ is substituted with one or two substituents independently selected from —CH₃, —OCH₃, CF₃, halo, —C(═O)—NH₂, and CN.

343. A compound according to numbered embodiment 338, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₃ is substituted with one or two substituents independently selected from —CH₃, —CH₂—OH, —CH₂—O—CH₃, —CH₂—NH₂, OH, NH₂, CF₃, halo, —C(═O)—NH₂, and CN.

344. A compound according to numbered embodiment 339, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₃ is substituted with one or two substituents independently selected from —CH₃, —CH₂—OH, —CH₂—O—CH₃, —CH₂—NH₂, OH, CF₃, halo, —C(═O)—NH₂, and CN.

345. A compound according to numbered embodiment 340, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₃ is substituted with one or two substituents independently selected from —CH₃, —CH₂—NH₂, CF₃, halo, —C(═O)—NH₂, and CN.

346. A compound according to numbered embodiment 341 or numbered embodiment 343, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₃ is substituted with one or two substituents independently selected from
—CH₃, —CH₂—OH, —CH₂—O—CH₃, OH, NH₂, CF₃, halo, —C(═O)—NH₂, and CN.

347. A compound according to numbered embodiment 341 or numbered embodiment 344, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₃ is substituted with one or two substituents independently selected from
—CH₃, —CH₂—OH, —CH₂—O—CH₃, OH, CF₃, halo, —C(═O)—NH₂, and CN.

348. A compound according to numbered embodiment 342 or numbered embodiment 345, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₃ is substituted with one or two substituents independently selected from
—CH₃, CF₃, halo, —C(═O)—NH₂, and CN.

349. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof, wherein, when R₃ is a six-membered ring substituted with two substituents, R₃ is substituted with a chlorine substituent at the meta-position to the attachment to B_{6.}

350. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof, wherein, when R₃ is substituted with two substituents, R₃ is substituted with a 3-chloro group.

351. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof, wherein when R₃ is substituted with one or two substituents independently selected from
—CH₃, —CH₂—OH, —CH₂—O—CH₃, —CH₂—NH₂, OH, NH₂, CF₃, halo, and CN.

352. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof, wherein when R₃ is substituted with one or two substituents independently selected from
—CH₃, —CH₂—OH, —CH₂—O—CH₃, —CH₂—NH₂, OH, CF₃, halo, and CN.

353. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof, wherein when R₃ is substituted with one or two substituents independently selected from
—CH₃, —CH₂—NH₂, CF₃, halo, and CN.

354. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof, wherein when R₃ is substituted with one or two substituents independently selected from
—CH₃, —CH₂—OH, —CH₂—O—CH₃, CF₃, OH, NH₂, halo, and CN.

355. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof, wherein when R₃ is substituted with one or two substituents independently selected from
—CH₃, —CH₂—OH, —CH₂—O—CH₃, CF₃, OH, halo, and CN.

356. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof, wherein when R₃ is substituted with one or two substituents independently selected from
—CH₃, CF₃, halo, and CN.

357. A compound according to any of numbered embodiments 1-315, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₃ is substituted with a small alkyl^{b} and optionally a further substituent selected from small alkyl^{b}, small alkoxy, cyclopropyl, OH, NH₂, CF₃, halo, —C(═O)—NH₂, —C(═O)—N(H)(small alkyl), —C(═O)—N(small alkyl)(small alkyl), and CN.

358. A compound according to any of numbered embodiments 1-315, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₃ is substituted with a small alkyl and optionally a further substituent selected from small alkyl^{b}, small alkoxy, cyclopropyl, OH, NH₂, CF₃, halo, —C(═O)—NH₂, —C(═O)—N(H)(small alkyl), —C(═O)—N(small alkyl)(small alkyl), and CN.

359. A compound according to any of numbered embodiments 1-315, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₃ is substituted with a small alkyl and optionally a further substituent selected from small alkyl, small alkoxy, cyclopropyl, OH, CF₃, halo, —C(═O)—NH₂, —C(═O)—N(H)(small alkyl), —C(═O)—N(small alkyl)(small alkyl), and CN.

360. A compound according to numbered embodiment 357 or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₃ is substituted with a (C₁-C₃)alkyl substituted with NH₂, and wherein R₃ is optionally substituted with a further substituent selected from small alkyl^{b}, small alkoxy, cyclopropyl, OH, NH₂, CF₃, halo, —C(═O)—NH₂, —C(═O)—N(H)(small alkyl), —C(═O)—N(small alkyl)(small alkyl), and CN.

361. A compound according to numbered embodiment 358 or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₃ is substituted with a (C₁-C₃)alkyl substituted with NH₂, and wherein R₃ is optionally substituted with a further substituent selected from small alkyl^{b}, small alkoxy, cyclopropyl, OH, CF₃, halo, —C(═O)—NH₂, —C(═O)—N(H)(small alkyl), —C(═O)—N(small alkyl)(small alkyl), and CN.

362. A compound according to numbered embodiment 359 or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₃ is substituted with a (C₁-C₃)alkyl substituted with NH₂, and wherein R₃ is optionally substituted with a further substituent selected from small alkyl, small alkoxy, cyclopropyl, OH, CF₃, halo, —C(═O)—NH₂, —C(═O)—N(H)(small alkyl), —C(═O)—N(small alkyl)(small alkyl), and CN.

363. A compound according to numbered embodiment 360, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₃ is substituted with-CH₂NH₂, and optionally a further substituent selected from small alkyl^{b}, small alkoxy, cyclopropyl, OH, NH₂, CF₃, halo, —C(═O)—NH₂, —C(═O)—N(H)(small alkyl), —C(═O)—N(small alkyl)(small alkyl), and CN.

364. A compound according to numbered embodiment 362, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₃ is substituted with-CH₂NH₂, and optionally a further substituent selected from small alkyl, small alkoxy, cyclopropyl, OH, CF₃, halo, —C(═O)—NH₂, —C(═O)—N(H)(small alkyl), —C(═O)—N(small alkyl)(small alkyl), and CN.

365. A compound according to any of numbered embodiments 357 to 364, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₃ is substituted with two substituents, wherein one of the substituents is-CH₂NH_{2.}

366. A compound according to any of numbered embodiments 319 to 354, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₃ is phenyl or pyridyl substituted with one or two halo substituents.

367. A compound according to any of numbered embodiments 319 to 354, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₃ is phenyl substituted with one or two halo substituents.

368. A compound according to numbered embodiment 366, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₃ is phenyl or pyridyl substituted with one or two substituents selected from F and Cl.

369. A compound according to numbered embodiment 367 or 368, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₃ is phenyl substituted with one or two substituents selected from F and Cl.

370. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof,
wherein R₃ is substituted with one substituent at the meta-position, labelled with a (*)

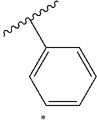 [SMILES: *c1ccccc1]

371. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₃ is substituted with one substituent at the meta-position, labelled with a (*), and one substituent at the ortho-position, labelled with a (+)

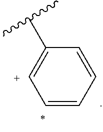 [SMILES: *C1=[C+]C=CC=C1]

372. A compound according to any of numbered embodiments 1 to 370, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₃ is substituted with one substituent at the meta-position, labelled with a (*), and one substituent at the meta-position, labelled with a (+)

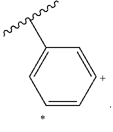 [SMILES: *C1=CC=C[C+]=C1]

373. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof,
wherein R₃ is unsubstituted at the para-position, labelled with a (˜)

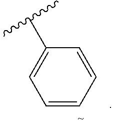 [SMILES: *c1ccccc1]

374. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof,
wherein R₃ is selected from:

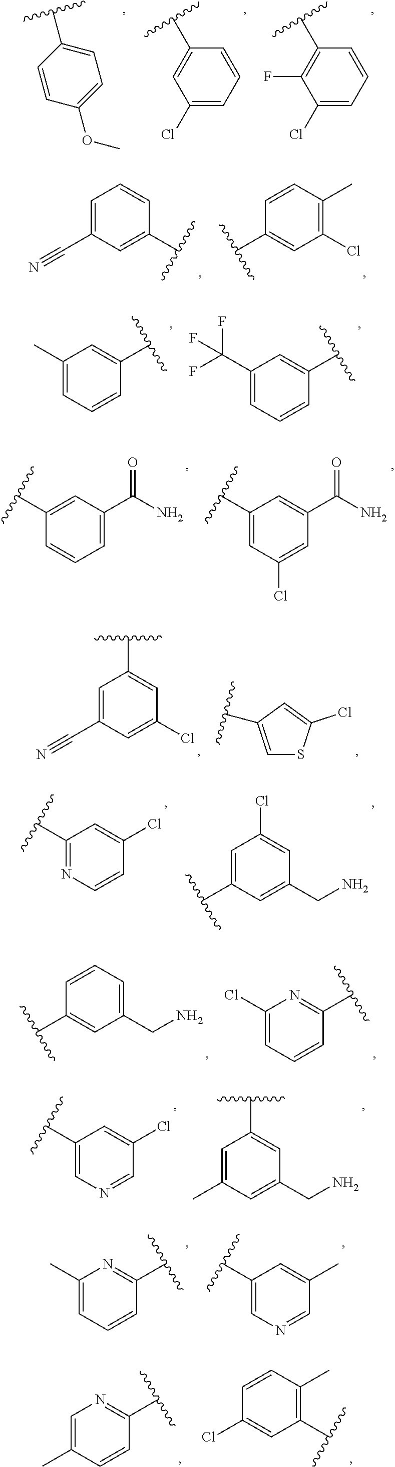

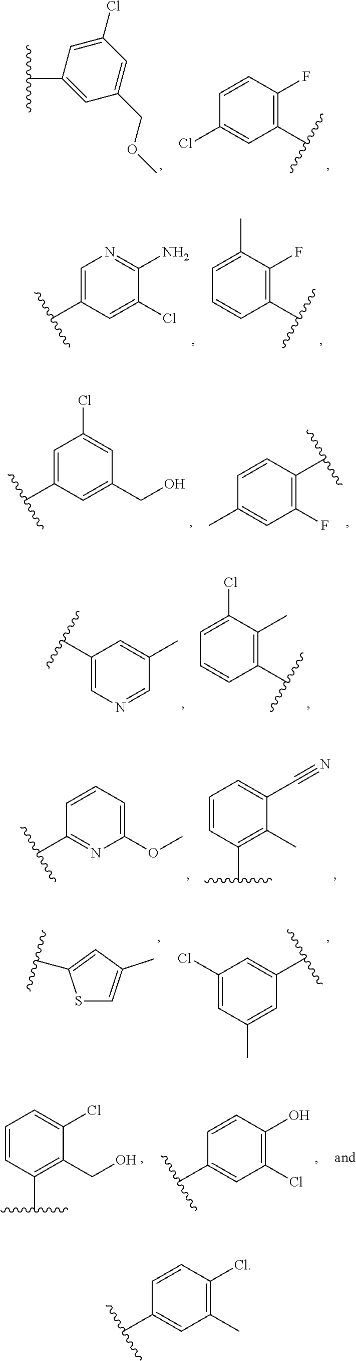

375. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof,
wherein R₃ is selected from:

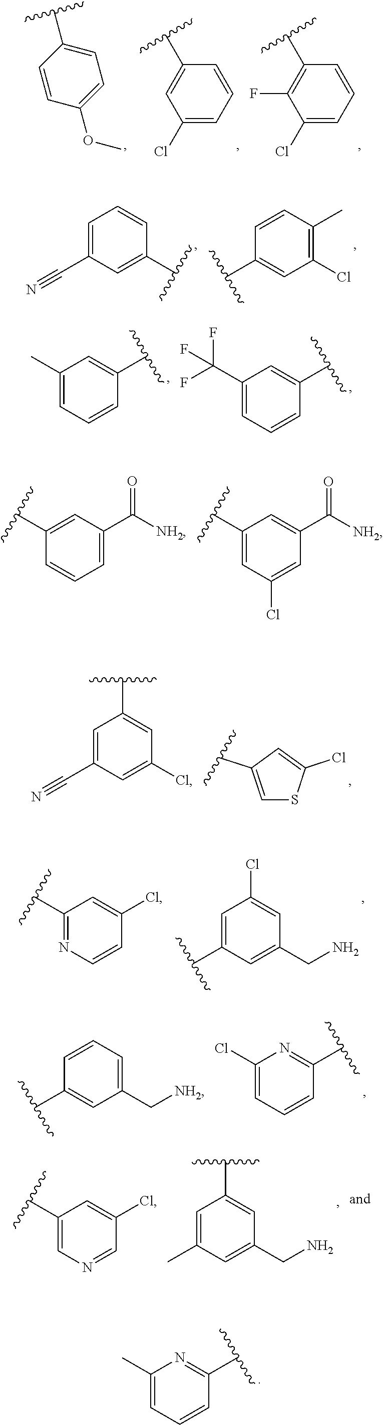

376. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof,
wherein R₃ is selected from:

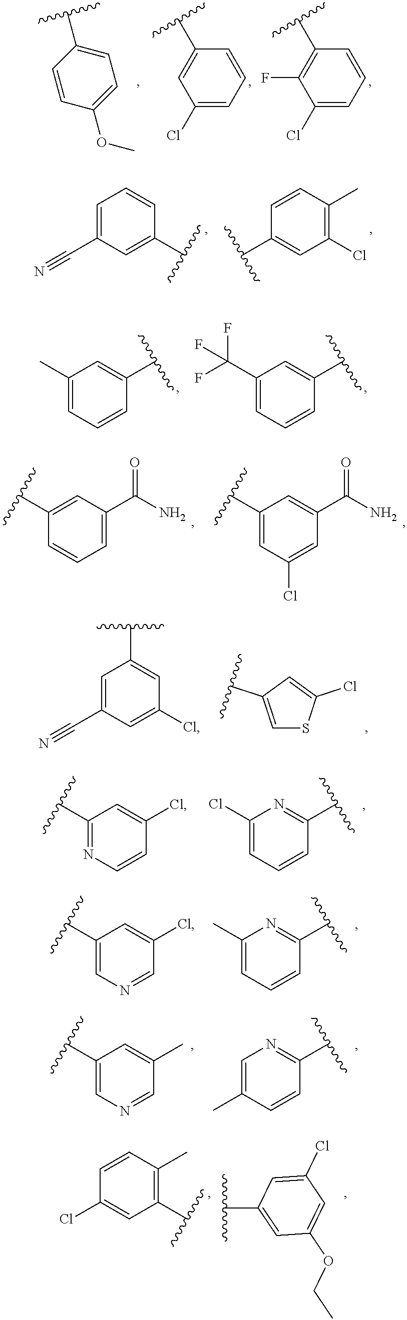

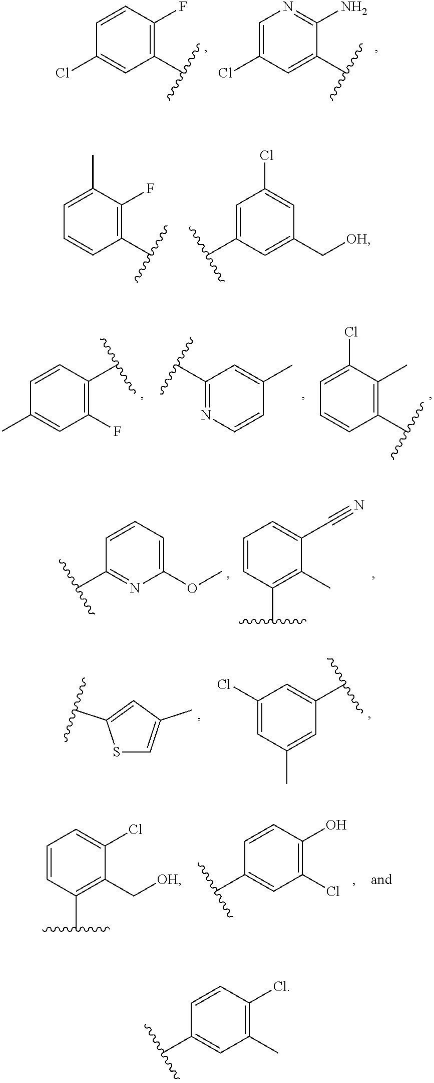

377. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof,
wherein R₃ is selected from:

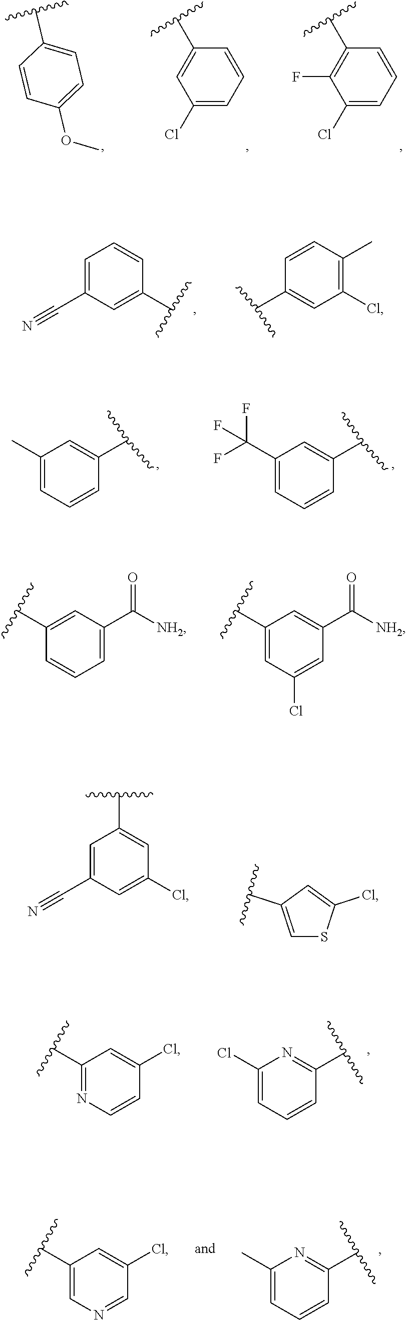

378. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof,
wherein R₃ is selected from:

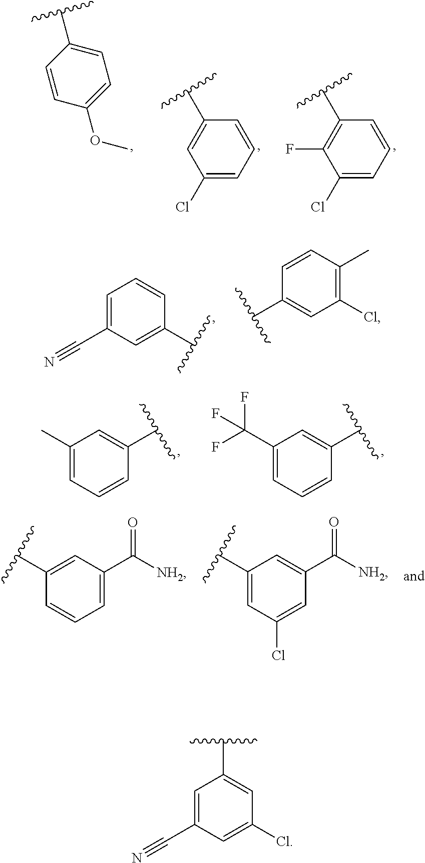

379. A compound according to any of numbered embodiments 1-374, or a tautomer, isomer, stereoisomer (including an enantiomer, a diastereoisomer and a racemic and scalemic mixture thereof), a deuterated analogue, and a pharmaceutically acceptable salt and/or solvate thereof,

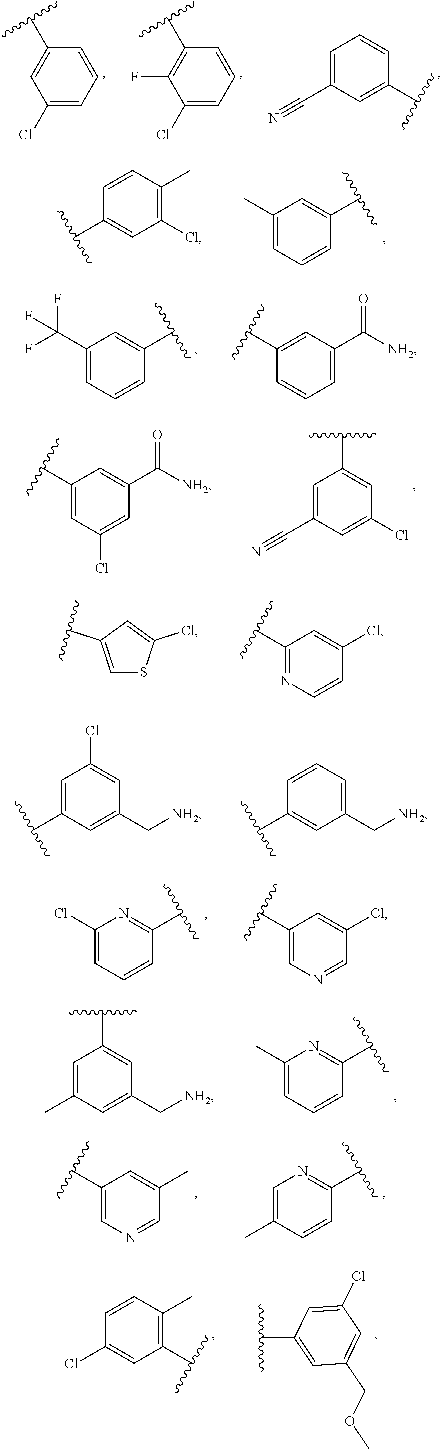

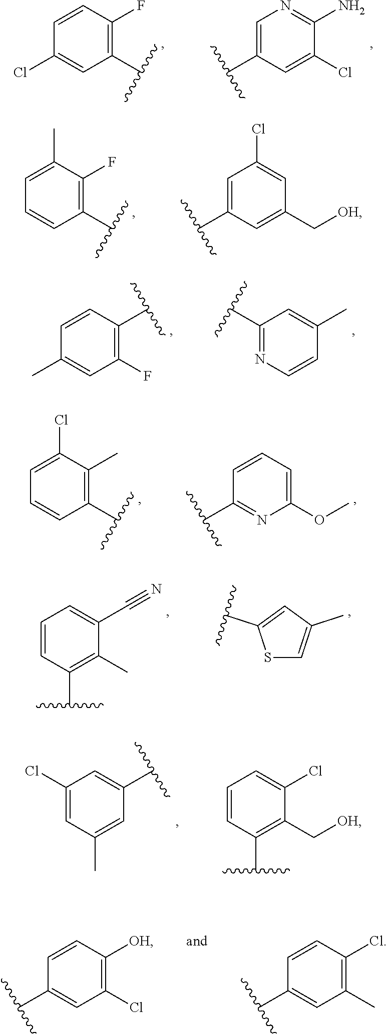

wherein R₃ is selected from:

380. A compound according to any of numbered embodiments 1-375, or a tautomer, isomer, stereoisomer (including an enantiomer, a diastereoisomer and a racemic and scalemic mixture thereof), a deuterated analogue, and a pharmaceutically acceptable salt and/or solvate thereof,
wherein R₃ is selected from:

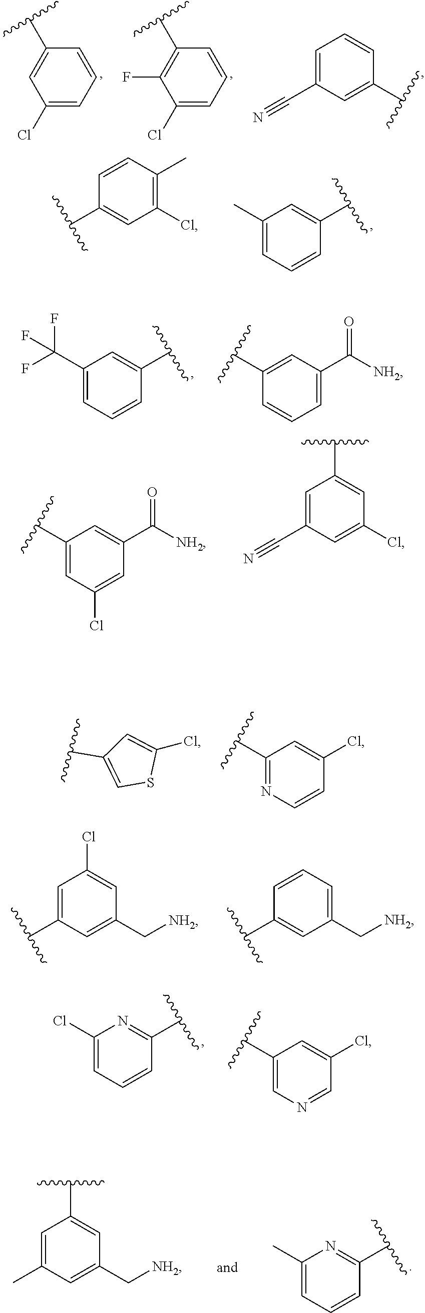

381. A compound according to any of numbered embodiments 1-374, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₃ is selected from:

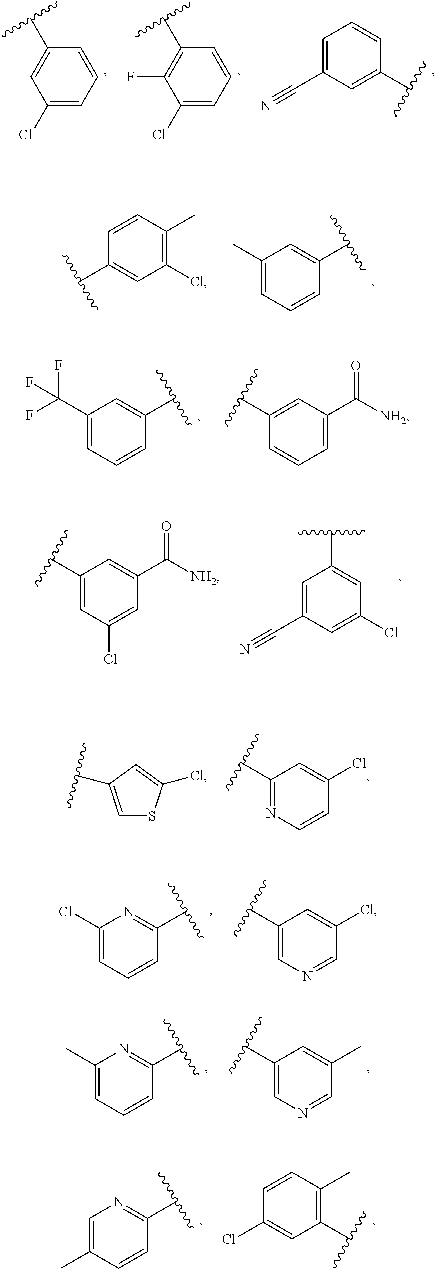

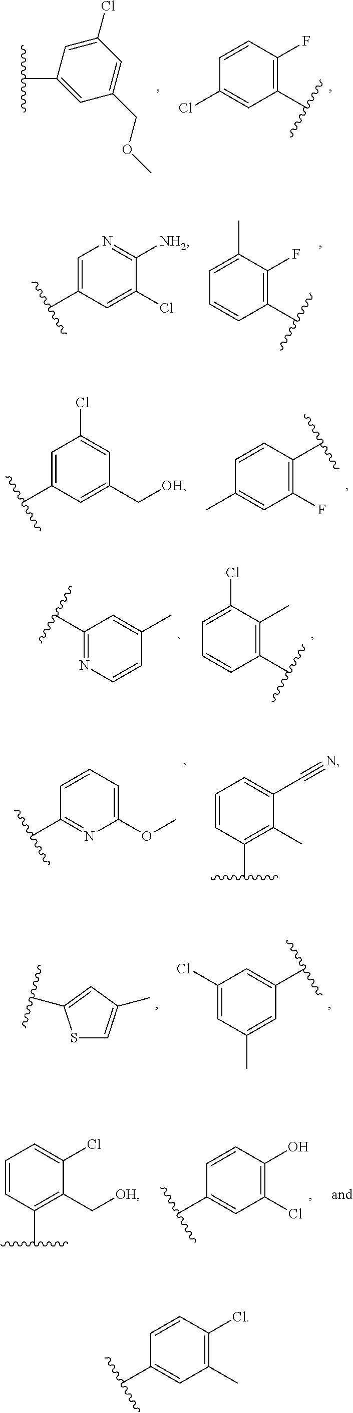

382. A compound according to any of numbered embodiments 1-375, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₃ is selected from:

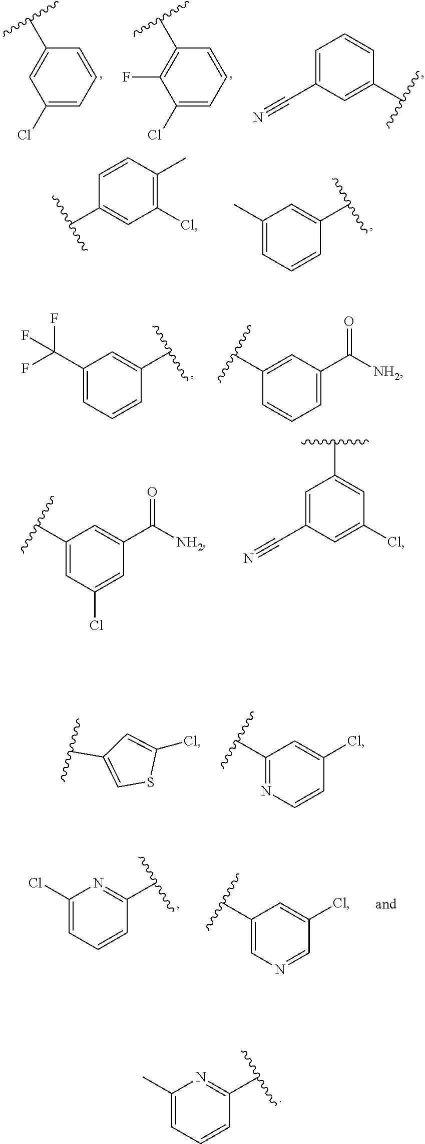

383. A compound according to any of numbered embodiments 1-376, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₃ is selected from:

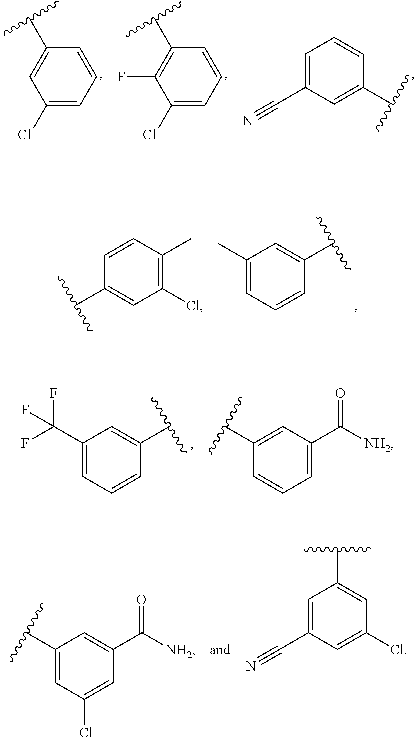

384. A compound according to any of numbered embodiments 1-378, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₃ is selected from:

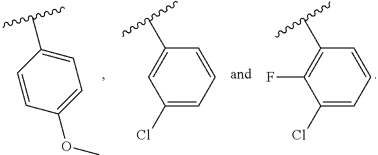

385. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof,
wherein R₃ is selected from:

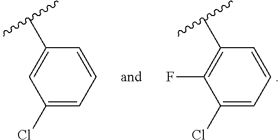 [SMILES: *c1cccc(Cl)c1 ; *c1cccc(Cl)c1F]

386. A compound according to any of numbered embodiments 1-374, or a tautomer, isomer, stereoisomer (including an enantiomer, a diastereoisomer and a racemic and scalemic mixture thereof), a deuterated analogue, and a pharmaceutically acceptable salt and/or solvate thereof,
wherein R₃ is selected from:

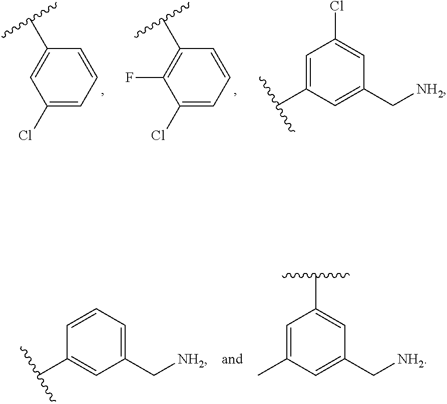

387. A compound according to any of numbered embodiments 1-375, or a tautomer, isomer, stereoisomer (including an enantiomer, a diastereoisomer and a racemic and scalemic mixture thereof), a deuterated analogue, and a pharmaceutically acceptable salt and/or solvate thereof,
wherein R₃ is selected from:

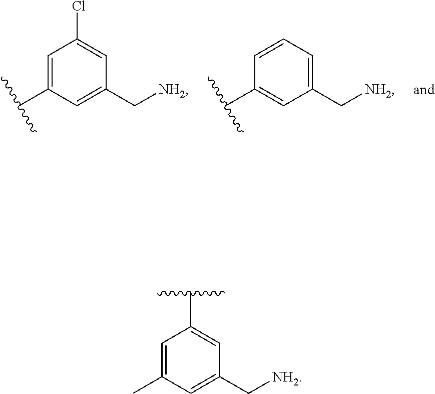

388. A compound according to numbered embodiment 387 or a tautomer, isomer, stereoisomer (including an enantiomer, a diastereoisomer and a racemic and scalemic mixture thereof), a deuterated analogue, and a pharmaceutically acceptable salt and/or solvate thereof,
wherein R₃ is selected from:

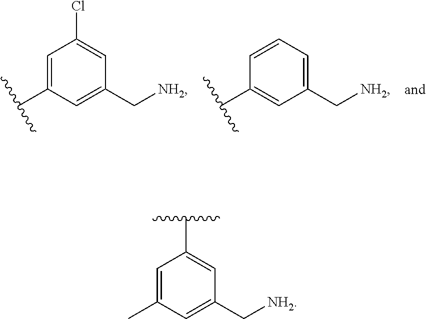

389. A compound according to any of the preceding numbered embodiments, or a tautomer, isomer, stereoisomer (including an enantiomer, a diastereoisomer and a racemic and scalemic mixture thereof), a deuterated analogue, and a pharmaceutically acceptable salt and/or solvate thereof,
wherein R₃ is

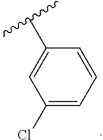 [SMILES: *c1cccc(Cl)c1]

390. A compound according to any preceding numbered embodiment, or a tautomer, isomer, stereoisomer (including an enantiomer, a diastereoisomer and a racemic and scalemic mixture thereof), a deuterated analogue, and a pharmaceutically acceptable salt and/or solvate thereof, which inhibits FXIIa.

391. A compound according to numbered embodiment 390, or a tautomer, isomer, stereoisomer (including an enantiomer, a diastereoisomer and a racemic and scalemic mixture thereof), a deuterated analogue, and a pharmaceutically acceptable salt and/or solvate thereof, which inhibits FXIIa with an IC50 value of <40,000 nM.

392. A compound according to numbered embodiment 391, or a tautomer, isomer, stereoisomer (including an enantiomer, a diastereoisomer and a racemic and scalemic mixture thereof), a deuterated analogue, and a pharmaceutically acceptable salt and/or solvate thereof, which inhibits FXIIa with an IC50 value of <10,000 nM.

393. A compound according to numbered embodiment 392, or a tautomer, isomer, stereoisomer (including an enantiomer, a diastereoisomer and a racemic and scalemic mixture thereof), a deuterated analogue, and a pharmaceutically acceptable salt and/or solvate thereof, which inhibits FXIIa with an IC50 value of <3,000 nM.

394. A compound according to numbered embodiment 393, or a tautomer, isomer, which inhibits FXIIa with an IC50 value of <1,000 nM.

395. A compound according to numbered embodiment 394, or a tautomer, isomer, stereoisomer (including an enantiomer, a diastereoisomer and a racemic and scalemic mixture thereof), a deuterated analogue, and a pharmaceutically acceptable salt and/or solvate thereof, which inhibits FXIIa with an IC50 value of <300 nM.

396. A compound according to numbered embodiment 395, or a tautomer, isomer, stereoisomer (including an enantiomer, a diastereoisomer and a racemic and scalemic mixture thereof), a deuterated analogue, and a pharmaceutically acceptable salt and/or solvate thereof, which inhibits FXIIa with an IC50 value of <100 nM.

397. A compound according to any preceding numbered embodiment, or a tautomer, isomer, stereoisomer (including an enantiomer, a diastereoisomer and a racemic and scalemic mixture thereof), a deuterated analogue, and a pharmaceutically acceptable salt and/or solvate thereof, which inhibits FXIIa selectively compared to inhibition of any other serine protease.

398. A compound according to numbered embodiment 397, or a tautomer, isomer, stereoisomer (including an enantiomer, a diastereoisomer and a racemic and scalemic mixture thereof), a deuterated analogue, and a pharmaceutically acceptable salt and/or solvate thereof, which inhibits FXIIa selectively compared to inhibition of any one of PKaI, KLK1, plasmin, FXa, FXIa, or thrombin.

399. A compound according to numbered embodiment 398, or a tautomer, isomer, stereoisomer (including an enantiomer, a diastereoisomer and a racemic and scalemic mixture thereof), a deuterated analogue, and a pharmaceutically acceptable salt and/or solvate thereof, which inhibits FXIIa selectively compared to inhibition of FXa.

400. A compound according to numbered embodiment 399, or a tautomer, isomer, which is at least 10-fold selective for inhibition of FXIIa compared to inhibition of FXa.

401. A compound according to numbered embodiment 400, or a tautomer, isomer, stereoisomer (including an enantiomer, a diastereoisomer and a racemic and scalemic mixture thereof), a deuterated analogue, and a pharmaceutically acceptable salt and/or solvate thereof, which is at least 100-fold selective for inhibition of FXIIa compared to inhibition of FXa.

402. A compound according to numbered embodiment 401, or a tautomer, isomer, stereoisomer (including an enantiomer, a diastereoisomer and a racemic and scalemic mixture thereof), a deuterated analogue, and a pharmaceutically acceptable salt and/or solvate thereof, which is at least 1,000-fold selective for inhibition of FXIIa compared to inhibition of FXa.

403. A compound according to numbered embodiment 402, or a tautomer, isomer, stereoisomer (including an enantiomer, a diastereoisomer and a racemic and scalemic mixture thereof), a deuterated analogue, and a pharmaceutically acceptable salt and/or solvate thereof, which is at least 10,000-fold selective for inhibition of FXIIa compared to inhibition of FXa.

404. A compound selected from Table 1a, Table 1b, Table 1c, Table 1d, Table 2a, Table 2b, Table 2c, Table 2d, Table 3a, Table 3b, Table 3c, Table 3d, and Table 4, or a pharmaceutically acceptable salt, solvate, or solvate of a salt thereof.

405. A compound selected from Table 1a, Table 1b, Table 1c, Table 2a, Table 2b, Table 2c, Table 3a, Table 3b, Table 3c, and Table 4, or a pharmaceutically acceptable salt, solvate, or solvate of a salt thereof.

406. A compound selected from Table 1a, Table 1b, Table 2a, Table 2b, Table 3a, Table 3b, and Table 4, or a pharmaceutically acceptable salt, solvate, or solvate of a salt thereof.

407. A compound selected from Table 1a, Table 2a, and Table 3a, or a pharmaceutically acceptable salt, solvate, or solvate of a salt thereof.

408. A pharmaceutical composition comprising: a compound, or a pharmaceutically acceptable salt and/or solvate thereof, according to any of the preceding numbered embodiments, and at least one pharmaceutically acceptable excipient.

409. A compound, or a pharmaceutically acceptable salt and/or solvate thereof, as defined in any of numbered embodiments 1 to 407, or the pharmaceutical composition according to numbered embodiment 408, for use in medicine.

410. A compound, or a pharmaceutically acceptable salt and/or solvate thereof, as defined in any of numbered embodiments 1 to 407, or the pharmaceutical composition according to numbered embodiment 408, for use in a method of treatment of a disease or condition in which Factor XIIa activity is implicated.

411. A compound, a pharmaceutically acceptable salt and/or solvate thereof, or a pharmaceutical composition for use as defined in numbered embodiment 410, wherein the disease or condition in which Factor XIIa activity is implicated is a bradykinin-mediated angioedema, wherein the bradykinin-mediated angioedema is hereditary angioedema.

412. A compound, a pharmaceutically acceptable salt and/or solvate thereof, or a pharmaceutical composition for use as defined in numbered embodiment 410, wherein the disease or condition in which Factor XIIa activity is implicated is a bradykinin-mediated angioedema, wherein the bradykinin-mediated angioedema is non hereditary.

413. A compound, a pharmaceutically acceptable salt and/or solvate thereof, or a pharmaceutical composition for use as defined in numbered embodiment 410, wherein the disease or condition in which Factor XIIa activity is implicated is selected from vascular hyperpermeability, stroke including ischemic stroke and haemorrhagic accidents; retinal edema; diabetic retinopathy; impaired visual acuity; DME; retinal vein occlusion; and AMD.

414. A compound, a pharmaceutically acceptable salt and/or solvate thereof, or a pharmaceutical composition for use as defined in numbered embodiment 410, wherein the disease or condition in which Factor XIIa activity is implicated is a thrombotic disorder.

415. A compound, a pharmaceutically acceptable salt and/or solvate thereof, or a pharmaceutical composition for use as defined in numbered embodiment 414, wherein the thrombotic disorder is thrombosis; thromboembolism caused by increased propensity of medical devices that come into contact with blood to clot blood; prothrombotic conditions such as disseminated intravascular coagulation (DIC), Venous thromboembolism (VTE), cancer associated thrombosis, complications caused by mechanical and bioprosthetic heart valves, complications caused by catheters, complications caused by ECMO, complications caused by LVAD, complications caused by dialysis, complications caused by CPB, sickle cell disease, joint arthroplasty, thrombosis induced to tPA, Paget-Schroetter syndrome and Budd-Chari syndrome; atherosclerosis; COVID-19; COVID-19 associated with SARS-COV-2 infection; acute respiratory distress syndrome (ARDS); idiopathic pulmonary fibrosis (IPF); rheumatoid arthritis (RA); and cold-induced urticarial autoinflammatory syndrome.

416. A compound, a pharmaceutically acceptable salt and/or solvate thereof, or a pharmaceutical composition for use as defined in numbered embodiment 410, wherein the disease or condition in which Factor XIIa activity is implicated is hereditary angioedema; COVID-19; COVID-19 associated with SARS-COV-2 infection; and idiopathic pulmonary fibrosis (IPF).

417. A compound, a pharmaceutically acceptable salt and/or solvate thereof, or a pharmaceutical composition for use as defined in numbered embodiment 410, wherein the disease or condition in which Factor XIIa activity is implicated is COVID-19.

418. A compound, a pharmaceutically acceptable salt and/or solvate thereof, or a pharmaceutical composition for use as defined in numbered embodiment 410, wherein the disease or condition in which Factor XIIa activity is implicated is COVID-19 associated with SARS-CoV-2 infection.

419. A compound, a pharmaceutically acceptable salt and/or solvate thereof, or a pharmaceutical composition for use as defined in numbered embodiment 410, wherein the disease or condition in which Factor XIIa activity is implicated is idiopathic pulmonary fibrosis (IPF).

420. A compound, a pharmaceutically acceptable salt and/or solvate thereof, or a pharmaceutical composition for use as defined in numbered embodiment 410, wherein the disease or condition in which Factor XIIa activity is implicated is neuroinflammation; neuroinflammatory/neurodegenerative disorders such as MS (multiple sclerosis); other neurodegenerative diseases such as Alzheimer's disease, epilepsy and migraine; sepsis; bacterial sepsis; inflammation; vascular hyperpermeability; and anaphylaxis.

421. A compound, a pharmaceutically acceptable salt and/or solvate thereof, or a pharmaceutical composition for use as defined in numbered embodiment 410, wherein the disease or condition in which Factor XIIa activity is implicated is obesity or diabetes.

422. A compound, a pharmaceutically acceptable salt and/or solvate thereof, or a pharmaceutical composition for use as defined in numbered embodiment 410, wherein the disease or condition in which Factor XIIa activity is implicated is kidney disease, renal fibrosis, glomerulosclerosis, renal scarring, ischemia/reperfusion injury in native or transplant kidneys, and acute kidney injury.

423. A compound, a pharmaceutically acceptable salt and/or solvate thereof, or a pharmaceutical composition for use as defined in numbered embodiment 410, wherein the disease or condition in which Factor XIIa activity is implicated is brain glioblastoma growth and malignancy, and complications thereof including oedema and cognitive function.

424. A compound, a pharmaceutically acceptable salt and/or solvate thereof, or a pharmaceutical composition for use as defined in numbered embodiment 410, wherein the disease or condition in which Factor XIIa activity is implicated is intradialytic hypotension (IDH).

### Numbered Paragraphs

1. A compound of Formula (I),

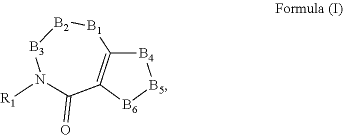

wherein:
B₁ is a carbon centre or nitrogen centre; and either:
i) B₂ and B₃ together form a single bond between B₁ and the nitrogen atom to which R₁ is attached,
(ii) B₂ is a carbon centre or a nitrogen centre, and B₃ is a single bond between B₂ and the nitrogen atom to which R₁ is attached,
(iii) B₂ is a carbon centre and B₃ is a nitrogen centre, or
(iv) B₂ and B₃ are both carbon centres,
wherein, when B₂ is a carbon centre or nitrogen centre, B₁ is connected to B₂ via either a single or double bond;
wherein, when both B₂ and B₃ are present, B₂ and B₃ are connected via either a single or double bond;
wherein B₁ may, where possible, be optionally substituted with 1 or 2 substituents selected from alkyl, cycloalkyl, alkoxy, OH, CF₃, CN, and halo, or 1 oxo substituent, or 2 substituents which, together with B₁ to which they are both attached, form a 3-, 4-, or 5-membered non-aromatic monocyclic carbon-containing ring, optionally wherein one or two ring members are selected from N, S, and O;
wherein, when B₂ is a carbon centre, B₂ may be optionally substituted with 1 or 2 substituents selected from alkyl, cycloalkyl, alkoxy, OH, CF₃, CN, and halo, or 1 oxo substituent; and
wherein, when B₃ is a carbon centre, B₃ may be optionally substituted with 1 or 2 substituents selected from alkyl, cycloalkyl, alkoxy, OH, CF₃, CN, and halo, or 1 oxo substituent;
and either:
(i) B₄ is N(R₂), B₅ is N, and B₆ is C(R₃), and B₄ is connected to B₅ via a single bond and B₅ is connected to B₆ via a double bond;
(ii) B₄ is C(R₂), B₅ is N, and B₆ is N(R₃), and B₄ is connected to B₅ via a double bond and B₅ is connected to B₆ via a single bond; or
(iii) B₄ is C(R₂), B₅ is C(H), and B₆ is N(R₃), and B₄ is connected to B₅ via a double bond and B₅ is connected to B₆ via a single bond;
R₂ is selected from H, —(CH₂)₀₋₃—C(═O)—N(R₄)(R₅), —C(═O)—O—R₆, —(CH₂)₀₋₃—NR₁₄—C(═O)R₁₅, —(CH₂)₀₋₃—NR₃₄—S(═O)₂R₃₅, alkyl, cycloalkyl, heterocycloalkyl, alkoxy, —OH, —CF₃, halo, aryl, heteroaryl, —CH₂—O—R₁₆ and —CN;
wherein R₄ is H, alkyl, cycloalkyl, and heterocycloalkyl, and
R₅ is selected from alkyl, —OH, —S(═O)₂-(small alkyl), —(CH₂)₁₋₃—(C═O)—OR₃₃, —(CH₂)₁₋₃—(C═O)—N(R₃₆)(R₃₇), —(CH₂)₀₋₃-cycloalkyl^{a}, —(CH₂)₀₋₃-heterocycloalkyl, —(CH(alkyl))-heterocycloalkyl, —(CH₂)₀₋₃-polycycloalkyl, —(C(H)(phenyl))-alkyl, —(CH₂)₀₋₃heteroaryl^{a}, —(C(H)(heteroaryl))-alkyl, —(C(H)(alkyl))-(C═O)—OR₃₈, —(C(H)(alkyl))-(C═O)—N(R₃₉)(R₄₀), —(C(H)(heteroaryl))-(C═O)—OR₂₈, and —(C(H)(heteroaryl))-(C═O)—N(R₄₁)(R₄₂);
or wherein R₄ and R₅ together with the nitrogen atom to which they are attached form a carbon-containing 4-, 5-, 6-, or 7-membered heterocyclic ring which may be saturated or unsaturated with 1 or 2 double bonds, optionally containing one or two additional heteroatoms selected from N, O, and S, wherein the ring may be optionally substituted with 1, 2, or 3 substituents selected from oxo, alkyl, alkoxy, OH, halo and CF_{3;}
R₂₈, R₃₃ R₃₆, R₃₇, R₃₈, R₃₉, R₄₀, R₄₁, and R₄₂ are each independently selected from H and small alkyl;
R₆ is selected from H, alkyl, —(CH₂)₀₋₃-cycloalkyl, —(CH₂)₀₋₃-heterocycloalkyl, —(CH(alkyl))-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, and —(C(H)(heteroaryl))-alkyl;
R₁₄ is selected from H, alkyl, cycloalkyl, and heterocycloalkyl;
R₁₅ is selected from H, alkyl, —(CH₂)₀₋₃-cycloalkyl, —(CH₂)₀₋₃-heterocycloalkyl, —(CH(alkyl))-heterocycloalkyl, —(CH₂)₀₋₃-aryl, —(C(H)(phenyl))-alkyl, —(CH₂)₀₋₃heteroaryl^{a}, and —(C(H)(heteroaryl))-alkyl;
R₁₆ is selected from H, alkyl, cycloalkyl, —(CH₂)₁₋₃—(C═O)—OH, heterocycloalkyl, and —(CH₂)₀₋₃heteroaryl;
R₃₄ is selected from H, alkyl, cycloalkyl, and heterocycloalkyl;
R₃₅ is selected from H, alkyl, —(CH₂)₀₋₃-cycloalkyl, —(CH₂)₀₋₃-heterocycloalkyl, —(CH(alkyl))-heterocycloalkyl, —(CH₂)₀₋₃-aryl, —(CH₂)₀₋₃heteroaryl^{a}, and —(C(H)(heteroaryl))-alkyl;
R₁ is:
(i) Formula (II), wherein Formula (II) is represented by Formula (IIb),

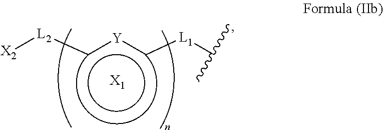

wherein:
n is 1;
L₁ is a linker selected from a single bond, —(CH₂)₁₋₃—, —(CH)(alkyl)-, and —C(alkyl)(alkyl)-;
Y is selected from C, N, O and S;
X₁ is phenyl or a 5- or 6-membered carbon-containing aromatic ring containing one, two or three ring members that are selected from N, S, and O;
wherein X₁ may be optionally substituted with 1 or 2 substituents independently selected from alkyl, cycloalkyl, alkoxy, OH, CF₃, and halo;
L₂ is a linker selected from a single bond, —(CH₂)₁₋₃—, —(CH)(alkyl)-, —C(alkyl)(alkyl)-, —C(═O)—, and —SO₂—; and
X₂ is either:
(a) —NR₂₀R₂₁,
wherein R₂₀ is selected from H, alkyl, and —(CH₂)₀₋₃—R_{22;}
wherein R₂₁ is selected from H, alkyl, and —(CH₂)₀₋₃—R₂₃;
wherein R₂₂ is selected from cycloalkyl, heterocycloalkyl, aryl, heteroaryl,

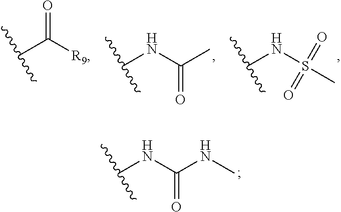

wherein R₂₃ is selected from cycloalkyl, heterocycloalkyl, aryl, heteroaryl,

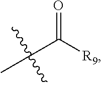

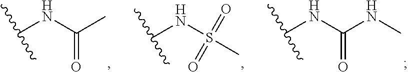

or
(b) 2-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O, and X₂ may be further optionally substituted with 1 or 2 substituents R₂₉ and R₃₀, wherein R₂₉ and R₃₀ are independently selected from alkyl, cycloalkyl, alkoxy, OH, oxo, CF₃, and halo; or,
(ii) Formula (III) comprising an aromatic ring A fused to a non-aromatic ring A′,

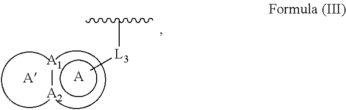

wherein:
L₃ is a linker selected from a single bond, —(CH₂)₁₋₃—, —(CH)(alkyl)-, and —C(alkyl)(alkyl);
A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, CF₃, and halo;
A′ is a non-aromatic carbon-containing monocyclic ring containing 5, 6, or 7, ring members, wherein at least one ring member is independently selected from N, S, and O;
wherein A′ may be optionally substituted with one R₇, wherein R₇ is selected from alkyl, alkoxy, and —(CH₂)₀₋₃—R₈,
wherein R₈ is selected from cycloalkyl, heterocycloalkyl, aryl, heteroaryl,

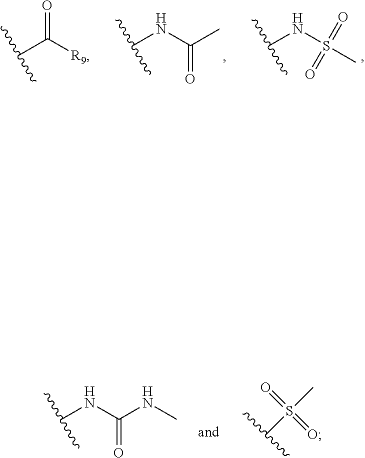

wherein R₉ is selected from H, alkyl, OH, N (R₃₁)(R₃₂), and alkoxy;
wherein R₃₁ is H or small alkyl;
wherein R₃₂ is selected from H, small alkyl, and —(CH₂)₀₋₃heteroaryl;
wherein A′ may be optionally substituted with 1, 2, or 3 further substituents R₁₀, R₁₁, and
R₁₇, wherein R₁₀, R₁₁, and R₁₇, are independently selected from alkyl, cycloalkyl, alkoxy, OH, Oxo, CF₃, and halo,
or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O; and
R₃ is phenyl, or a 5- or 6-membered carbon-containing aromatic ring containing one, two or three ring members that are selected from N, S, O, and
wherein R₃ is substituted with one or two substituents independently selected from small alkyl, small alkoxy, cyclopropyl, OH, CF₃, halo, —C(═O)—NH₂, —C(═O)—N(H)(small alkyl), —C(═O)—N(small alkyl)(small alkyl), and CN; wherein R₃ is not

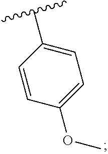 [SMILES: *c1ccc(OC)cc1]

and
alkyl is a linear saturated hydrocarbon having up to 10 carbon atoms (C₁-C₁₀) or a branched saturated hydrocarbon of between 3 and 10 carbon atoms (C₃-C₁₀); alkyl may optionally be substituted with 1, 2 or 3 substituents independently selected from cyclopropyl, (C₁-C₆) alkoxy, OH, —NR₁₂R₁₃, —C(═O) NR₁₂R₁₃, CN, CF₃, and halo;
small alkyl is a linear saturated hydrocarbon having up to 4 carbon atoms (C₁-C₄) or a branched saturated hydrocarbon of between 3 and 4 carbon atoms (C₃-C₄); small alkyl may optionally be substituted with 1 or 2 substituents independently selected from cyclopropyl, OH, CN, CF₃, NH₂, —C(═O)NH₂, and halo;
alkoxy is OCF₃, a linear O-linked hydrocarbon of between 1 and 6 carbon atoms (C₁-C₆), or a branched O-linked hydrocarbon of between 3 and 6 carbon atoms (C₃-C₆); alkoxy may optionally be substituted with 1 or 2 substituents independently selected from cyclopropyl, OH, CN, CF₃, and halo;
small alkoxy is OCF₃, a linear O-linked hydrocarbon of between 1 and 4 carbon atoms (C₁-C₄), or a branched O-linked hydrocarbon of between 3 and 4 carbon atoms (C₃-C₄); small alkoxy may optionally be substituted with 1 or 2 substituents independently selected from cyclopropyl, OH, CN, CF₃, and halo;
aryl is phenyl, biphenyl or naphthyl; aryl may be optionally substituted with 1, 2 or 3 substituents independently selected from alkyl, alkoxy, methylenedioxy, ethylenedioxy, OH, halo, CN, —(CH₂)₀₋₃—NR₁₂R₁₃, OCF₃ and CF_{3;}
polycycloalkyl is a bicyclic saturated hydrocarbon ring system of between 5 and 8 carbon atoms (C₅-C₈), wherein the bicyclic ring is bridged, or is adamantyl; polycycloalkyl may optionally be substituted with 1 or 2 substituents independently selected from alkyl, (C₁-C₆) alkoxy, OH, CN, CF₃, and halo;
cycloalkyl is a monocyclic saturated hydrocarbon ring of between 3 and 6 carbon atoms (C₃-C₆); cycloalkyl may optionally be substituted with 1 or 2 substituents independently selected from alkyl, (C₁-C₆) alkoxy, OH, CN, CF₃, and halo
cycloalkyl^{a} is a monocyclic saturated hydrocarbon ring of between 3 and 6 carbon atoms (C₃-C₆); cycloalkyl may optionally be substituted with 1 or 2 substituents independently selected from alkyl, (C₁-C₆) alkoxy, OH, CN, CF₃, halo, and —(C═O)—OH;
halo is F, Cl, Br, or I;
heteroaryl is a 5- or 6-membered carbon-containing aromatic ring containing one, two or three ring members that are selected from N, S, and O; wherein heteroaryl may be optionally substituted with 1, 2 or 3 substituents independently selected from alkyl, alkoxy, cyclopropyl, OH, halo, CN, and CF_{3;}
heteroaryl^{a} is a 5-, 6-, 9- or 10-membered mono- or bi-cyclic aromatic ring containing, where possible, 1, 2, 3 or 4 ring members independently selected from N, S, and O; wherein heteroaryl^{a} may be optionally substituted with 1, 2 or 3 substituents independently selected from alkyl, alkoxy, cyclopropyl, OH, halo, CN, and CF_{3;}
heterocycloalkyl is a non-aromatic carbon-containing mono- or bi-cyclic ring containing 3, 4, 5, 6, or 7 ring members, wherein at least one ring member is independently selected from N, S, and O; heterocycloalkyl may optionally be substituted with 1 or 2 substituents independently selected from alkyl, cyclopropyl, (C₁-C₅) alkoxy, OH, CN, CF₃, oxo, and halo; wherein when heterocycloalkyl is a bi-cyclic ring system, the bicyclic ring system is fused, bridged or spiro;
each R₁₂ and R₁₃ is independently selected from H, alkyl, cycloalkyl, and heterocycloalkyl;
and tautomers, isomers, stereoisomers (including enantiomers, diastereoisomers and racemic and scalemic mixtures thereof), deuterated analogues, and pharmaceutically acceptable salts and/or solvates thereof.

2. A compound according to numbered paragraph 1, or a tautomer, isomer, stereoisomer (including an enantiomer, a diastereoisomer and a racemic and scalemic mixture thereof), a deuterated analogue, and a pharmaceutically acceptable salt and/or solvate thereof, wherein:

(i) B₂ and B₃ together form a single bond between B₁ and the nitrogen atom to which R₁ is attached, or
(ii) B₂ is a carbon centre or a nitrogen centre and B₃ is a single bond between B₂ and the nitrogen atom to which R₁ is attached.

3. A compound according to any of the preceding numbered paragraphs, or a tautomer, isomer, stereoisomer (including an enantiomer, a diastereoisomer and a racemic and scalemic mixture thereof), a deuterated analogue, and a pharmaceutically acceptable salt and/or solvate thereof, wherein:
(i) B₁ is a carbon centre, and B₂ and B₃ together form a single bond between B₁ and the nitrogen atom to which R₁ is attached,
(ii) B₁ is a carbon centre and B₂ is a carbon centre, and B₁ is connected to B₂ via a single bond, or
(iii) B₁ is a nitrogen centre and B₂ is a carbon centre and B₁ is connected to B₂ via a double bond.

4. A compound according to numbered paragraph 3, or a tautomer, isomer, stereoisomer (including an enantiomer, a diastereoisomer and a racemic and scalemic mixture thereof), a deuterated analogue, a acceptable salt and/or and pharmaceutically solvate thereof, wherein B₁ is a carbon centre and B₂ is a carbon centre, and B₁ is connected to B₂ via a single bond, or wherein B₁ is a nitrogen centre and B₂ is a carbon centre and B₁ is connected to B₂ via a double bond.

5. A compound according to any of the preceding numbered paragraphs, or a tautomer, isomer, stereoisomer (including an enantiomer, a diastereoisomer and a racemic and scalemic mixture thereof), a deuterated analogue, and a pharmaceutically acceptable salt and/or solvate thereof, wherein:
(i) B₄ is N(R₂), B₅ is N, and B₆ is C(R₃), and B₄ is connected to B₅ via a single bond and B₅ is connected to B₆ via a double bond; or
(ii) B₄ is C(R₂), B₅ is N, and B₆ is N(R₃), and wherein B₄ is connected to B₅ via a double bond and B₅ is connected to B₆ via a single bond.

6. A compound according to any of the preceding numbered paragraphs, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₂ is selected from —C(═O)—N(R₄)(R₅), —C(═O)—O—R₆, —CH₂—NH—C(═O)CH₃, —CH₂—NH—S(═O)₂(cyclopropyl), —CH₂—O—R₁₆, —CH₂—OH, —CN, and

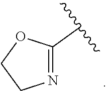 [SMILES: *C1=NCCO1]

7. A compound according to any of the preceding numbered paragraphs, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₂ is selected from —C(═O)—N(R₄)(R₅), —C(═O)—O—R₆, —CH₂—NH—C(═O)CH₃, —CH₂—NH—S(═O)₂(cyclopropyl), —CH₂—O—R₁₆, —CH₂—OH, and

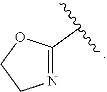 [SMILES: *C1=NCCO1]

8. A compound according to any of the preceding numbered paragraphs, or a tautomer, isomer, stereoisomer (including an enantiomer, a diastereoisomer and a racemic and scalemic mixture thereof), a deuterated analogue, and a pharmaceutically acceptable salt and/or solvate thereof, wherein:
R₄ is H or small alkyl, and
R₅ is selected from alkyl, —OH, —S(═O)₂-(small alkyl), —(CH₂)₁₋₃—(C═O)—OH, —(CH₂)₀₋₃-cycloalkyl^{a}, —(CH₂)₀₋₃-heterocycloalkyl, —(CH(alkyl))-heterocycloalkyl, —(CH₂)₀₋₃-polycycloalkyl, —(CH₂)₀₋₃-aryl, —(C(H)(phenyl))-alkyl, —(CH₂)₀₋₃-heteroaryl^{a}, —(C(H)(heteroaryl))-alkyl, —(C(H)(alkyl))—(C═O)—OH, and —(C(H)(heteroaryl))-(C═O)—OR_{28;}
or wherein R₄ and R₅ together with the nitrogen atom to which they are attached form a carbon-containing 4-, 5-, or 6-membered saturated heterocyclic ring, optionally containing an additional heteroatom selected from N, and O, which may be optionally mono- or di-substituted with substituents selected from oxo, alkyl, alkoxy, OH, halo and CF₃; and
R₆, where present, is selected from H, alkyl, —(CH₂)₀₋₃-cycloalkyl, —(CH₂)₀₋₃-heterocycloalkyl, —(CH(alkyl)-heterocycloalkyl, —(CH₂)₀₋₃-polycycloalkyl, —(CH₂)₀₋₃-aryl, —(C(H)(phenyl)-alkyl, —(CH₂)₀₋₃heteroaryl, and —(C(H)(heteroaryl)-alkyl.

9. A compound according to any of the preceding numbered paragraphs, or a tautomer, isomer, stereoisomer (including an enantiomer, a diastereoisomer and a racemic and scalemic mixture thereof), a deuterated analogue, and a pharmaceutically acceptable salt and/or solvate thereof, wherein X₁ is selected from phenyl and pyridyl.

10. A compound according to any of the preceding numbered paragraphs, or a tautomer, isomer, stereoisomer (including an enantiomer, a diastereoisomer and a racemic and scalemic mixture thereof), a deuterated analogue, and a pharmaceutically acceptable salt and/or solvate thereof, wherein Formula (II) is represented by Formula (IIa),

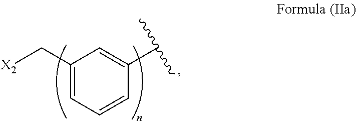

wherein:
n is 0 or 1;
X₂ is a non-aromatic, carbon-containing mono- or bi-cyclic ring containing 5, 6, or 7 ring members, wherein at least one ring member is independently selected from N, S, and O; wherein X₂ may be optionally substituted with 1 or 2 substituents R₁₈ and R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O.

11. A compound according to any of the preceding numbered paragraphs, or a tautomer, isomer, stereoisomer (including an enantiomer, a diastereoisomer and a racemic and scalemic mixture thereof), a deuterated analogue, and a pharmaceutically acceptable salt and/or solvate thereof, wherein A is phenyl or a 6-membered carbon-containing aromatic ring containing one, two or three nitrogen ring members.

12. A compound according to any of the preceding numbered paragraphs, or a tautomer, isomer, stereoisomer (including an enantiomer, a diastereoisomer and a racemic and scalemic mixture thereof), a deuterated analogue, and a pharmaceutically acceptable salt and/or solvate thereof, wherein A′ is a non-aromatic carbon-containing monocyclic ring containing 5, 6, or 7 ring members, wherein at least one ring member is N.

13. A compound according to any of the preceding numbered paragraphs, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₇, when present, is bonded to A′ via a N ring member.

14. A compound according to any of the preceding numbered paragraphs, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₇ is (C₁-C₃)alkyl.

15. A compound according to any of the preceding numbered paragraphs, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₃ is not substituted with a 4-methoxy group.

16. A compound according to any of the preceding numbered paragraphs, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₃ is selected from substituted phenyl, substituted pyridyl, and substituted thiophene.

17. A compound according to any of the preceding numbered paragraphs, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₃ is substituted with one or two substituents independently selected from small alkyl, small alkoxy, CF₃, halo, —C(═O)—NH₂, and CN.

18. A compound according to any of the preceding numbered paragraphs, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₃ is

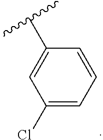 [SMILES: *c1cccc(Cl)c1]

19. A compound selected from Table 1a, Table 1b, Table 1c, Table 2a, Table 2b, Table 2c, Table 3a, Table 3b, Table 3c and Table 4, or a pharmaceutically acceptable salt, solvate, or solvate of a salt thereof.

20. A pharmaceutical composition comprising: a compound, or a pharmaceutically acceptable salt and/or solvate thereof, according to any of the preceding numbered paragraphs, and at least one pharmaceutically acceptable excipient.

21. A compound, or a pharmaceutically acceptable salt and/or solvate thereof, as defined in any of numbered paragraphs 1 to 19, or the pharmaceutical composition according to numbered paragraph 20, for use in medicine.

22. A compound, or a pharmaceutically acceptable salt and/or solvate thereof, as defined in any of numbered paragraphs 1 to 19, or the pharmaceutical composition according to numbered paragraph 20, for use in a method of treatment of a disease or condition in which Factor XIIa activity is implicated; wherein the disease or condition in which Factor XIIa activity is implicated is a bradykinin-mediated angioedema; wherein the bradykinin-mediated angioedema is hereditary angioedema.

23. A compound, or a pharmaceutically acceptable salt and/or solvate thereof, as defined in any of numbered paragraphs 1 to 19, or the pharmaceutical composition according to numbered paragraph 20, for use in a method of treatment of a disease or condition in which Factor XIIa activity is implicated; wherein the disease or condition in which Factor XIIa activity is implicated is a bradykinin-mediated angioedema; wherein the bradykinin-mediated angioedema is non hereditary.

24. A compound, or a pharmaceutically acceptable salt and/or solvate thereof, as defined in any of numbered paragraphs 1 to 19, or the pharmaceutical composition according to numbered paragraph 20, for use in a method of treatment of a disease or condition in which Factor XIIa activity is implicated; wherein the disease or condition in which Factor XIIa activity is implicated is a thrombotic disorder.

### Numbered Aspects

1. A compound of Formula (I),

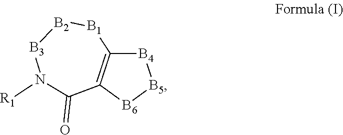

wherein:
B₁ is a carbon centre or nitrogen centre; and either:
(i) B₂ and B₃ together form a single bond between B₁ and the nitrogen atom to which R₁ is attached,
(ii) B₂ is a carbon centre or a nitrogen centre, and B₃ is a single bond between B₂ and the nitrogen atom to which R₁ is attached,
(iii) B₂ is a carbon centre and B₃ is a nitrogen centre, or
(iv) B₂ and B₃ are both carbon centres,
wherein, when B₂ is a carbon centre or nitrogen centre, B₁ is connected to B₂ via either a single or double bond;
wherein, when both B₂ and B₃ are present, B₂ and B₃ are connected via either a single or double bond;
wherein B₁ may, where possible, be optionally substituted with 1 or 2 substituents selected from alkyl, cycloalkyl, alkoxy, OH, CF₃, CN, and halo, or 1 oxo substituent, or 2 substituents which, together with B₁ to which they are both attached, form a 3-, 4-, or 5-membered non-aromatic monocyclic carbon-containing ring, optionally wherein one or two ring members are selected from N, S, and O;
wherein, when B₂ is a carbon centre, B₂ may be optionally substituted with 1 or 2 substituents selected from alkyl, cycloalkyl, alkoxy, OH, CF₃, CN, and halo, or 1 oxo substituent; and
wherein, when B₃ is a carbon centre, B₃ may be optionally substituted with 1 or 2 substituents selected from alkyl, cycloalkyl, alkoxy, OH, CF₃, CN, and halo, or 1 oxo substituent;
and either:
(i) B₄ is N(R₂), B₅ is N, and B₆ is C(R₃), and B₄ is connected to B₅ via a single bond and B₅ is connected to B₆ via a double bond;
(ii) B₄ is C(R₂), B₅ is N, and B₆ is N(R₃), and B₄ is connected to B₅ via a double bond and B₅ is connected to B₆ via a single bond; or
(iii) B₄ is C(R₂), B₅ is C(H), and B₆ is N(R₃), and B₄ is connected to B₅ via a double bond and B₅ is connected to B₆ via a single bond;
R₂ is selected from H, —(CH₂)₀₋₃—C(═O)—N(R₄)(R₅), —C(═O)—O—R₆, —(CH₂)₀₋₃—NR₁₄—C(═O)R₁₅, —(CH₂)₀₋₃—NR₃₄—S(═O)₂R₃₅, alkyl, cycloalkyl, heterocycloalkyl, alkoxy, —OH, —CF₃, halo, aryl, heteroaryl, —CH₂—O—R₁₆ and —CN;
wherein R₄ is H, alkyl, cycloalkyl, and heterocycloalkyl, and
R₅ is selected from alkyl, —(CH₂)₀₋₃-cycloalkyl, —(CH₂)₀₋₃-heterocycloalkyl, —(CH(alkyl))-heterocycloalkyl, —(CH₂)₀₋₃-polycycloalkyl, —(CH₂)₀₋₃-aryl, —(C(H)(phenyl))-alkyl, —(CH₂)₀₋₃heteroaryl^{a}, and —(C(H)(heteroaryl))-alkyl;
or wherein R₄ and R₅ together with the nitrogen atom to which they are attached form a carbon-containing 4-, 5-, 6-, or 7-membered heterocyclic ring which may be saturated or unsaturated with 1 or 2 double bonds, optionally containing one or two additional heteroatoms selected from N, O, and S, wherein the ring may be optionally substituted with 1, 2, or 3 substituents selected from oxo, alkyl, alkoxy, OH, halo and CF_{3;}
R₆ is selected from H, alkyl, —(CH₂)₀₋₃-cycloalkyl, —(CH₂)₀₋₃-heterocycloalkyl, —(CH(alkyl))-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, and —(C(H)(heteroaryl))-alkyl;
R₁₄ is selected from H, alkyl, cycloalkyl, and heterocycloalkyl;
R₁₅ is selected from H, alkyl, —(CH₂)₀₋₃-cycloalkyl, —(CH₂)₀₋₃-heterocycloalkyl, —(CH(alkyl))-heterocycloalkyl, —(CH₂)₀₋₃-aryl, —(C(H)(phenyl))-alkyl, —(CH₂)₀₋₃heteroaryl^{a}, and —(C(H)(heteroaryl))-alkyl;
R₁₆ is selected from H, alkyl, cycloalkyl, and heterocycloalkyl;
R₃₄ is selected from H, alkyl, cycloalkyl, and heterocycloalkyl;
R₃₅ is selected from H, alkyl, —(CH₂)₀₋₃-cycloalkyl, —(CH₂)₀₋₃-heterocycloalkyl, —(CH(alkyl))-heterocycloalkyl, —(CH₂)₀₋₃-aryl, —(C(H)(phenyl))-alkyl, —(CH₂)₀₋₃heteroaryl^{a}, and —(C(H)(heteroaryl))-alkyl;
R₁ is:
(i) Formula (II), wherein Formula (II) is represented by Formula (IIb),

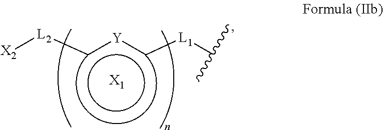

wherein:
n is 1;
L₁ is a linker selected from a single bond, —(CH₂)₁₋₃—, —(CH)(alkyl)-, and —C(alkyl)(alkyl)-;
Y is selected from C, N, O and S;
X₁ is phenyl or a 5- or 6-membered carbon-containing aromatic ring containing one, two or three ring members that are selected from N, S, and O;
wherein X₁ may be optionally substituted with 1 or 2 substituents independently selected from alkyl, cycloalkyl, alkoxy, OH, CF₃, and halo;
L₂ is a linker selected from a single bond, —(CH₂)₁₋₃—, —(CH)(alkyl)-, —C(alkyl)(alkyl)-, —C(═O)—, and —SO₂—; and
X₂ is either:
(a) NR₂₀R₂₁,
wherein R₂₀ is selected from H, alkyl, and —(CH₂)₀₋₃—R_{22;}
wherein R₂₁ is selected from H, alkyl, and —(CH₂)₀₋₃—R₂₃;
wherein R₂₂ is selected from cycloalkyl, heterocycloalkyl, aryl, heteroaryl,

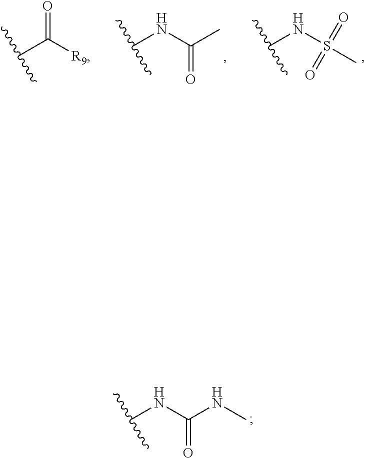

wherein R₂₃ is selected from cycloalkyl, heterocycloalkyl, aryl, heteroaryl,

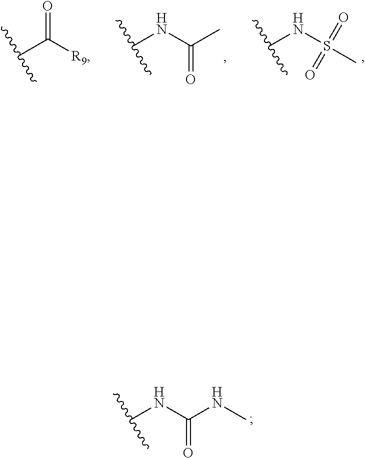

or
(b) 2-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected from alkyl, cycloalkyl, alkoxy, OH, oxo, CF₃, and halo,
   or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O; or,
(ii) Formula (III) comprising an aromatic ring A fused to a non-aromatic ring A′,

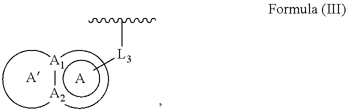

wherein:
L₃ is a linker selected from a single bond, —(CH₂)₁₋₃—, —(CH)(alkyl)-, and —C(alkyl)(alkyl);
A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, CF₃, and halo;
A′ is a non-aromatic carbon-containing monocyclic ring containing 5, 6, or 7, ring members, wherein at least one ring member is independently selected from N, S, and O; wherein A′ may be optionally substituted with one R₇, wherein R₇ is selected from alkyl, alkoxy, and —(CH₂)₀₋₃—R₈,
wherein R₈ is selected from cycloalkyl, heterocycloalkyl, aryl, heteroaryl,

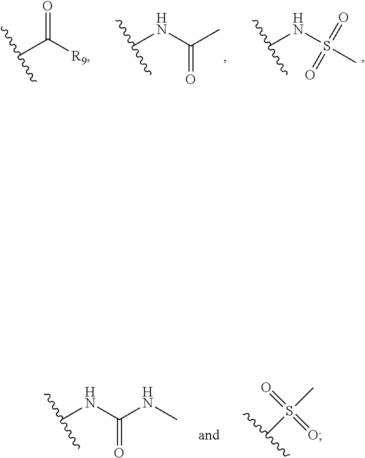

wherein R₉ is selected from H, alkyl, OH, NH₂, and alkoxy;
wherein A′ may be optionally substituted with 1, 2, or 3 further substituents R₁₀, R₁₁, and
R₁₇, wherein R₁₀, R₁₁, and R₁₇, are independently selected from alkyl, cycloalkyl, alkoxy, OH, Oxo, CF₃, and halo,
or wherein A′ may be optionally substituted with 1 or 2 further substituents R₂₄ and R₂₇ independently selected from alkyl, cycloalkyl, alkoxy, OH, oxo, CF₃, and halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are selected from N, S, and O; and
R₃ is phenyl, or a 5- or 6-membered carbon-containing aromatic ring containing one, two or three ring members that are selected from N, S, and O, wherein R₃ is substituted with one or two substituents independently selected from small alkyl, small alkoxy, cyclopropyl, OH, CF₃, halo, —C(═O)—NH₂, —C(═O)—N(H)(small alkyl),
—C(═O)—N(small alkyl)(small alkyl), and CN; wherein R₃ is not

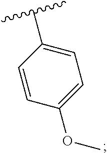 [SMILES: *c1ccc(OC)cc1]

and
alkyl is a linear saturated hydrocarbon having up to 10 carbon atoms (C₁-C₁₀) or a branched saturated hydrocarbon of between 3 and 10 carbon atoms (C₃-C₁₀); alkyl may optionally be substituted with 1, 2 or 3 substituents independently selected from cyclopropyl, (C₁-C₆) alkoxy, OH, —NR₁₂R₁₃, —C(═O) NR₁₂R₁₃, CN, CF₃, and halo;
small alkyl is a linear saturated hydrocarbon having up to 4 carbon atoms (C₁-C₄) or a branched saturated hydrocarbon of between 3 and 4 carbon atoms (C₃-C₄); small alkyl may optionally be substituted with 1 or 2 substituents independently selected from cyclopropyl, OH, CN, CF₃, NH₂, —C(═O)NH₂, and halo;
alkoxy is OCF₃, a linear O-linked hydrocarbon of between 1 and 6 carbon atoms (C₁-C₆), or a branched O-linked hydrocarbon of between 3 and 6 carbon atoms (C₃-C₆); alkoxy may optionally be substituted with 1 or 2 substituents independently selected from cyclopropyl, OH, CN, CF₃, and halo;
small alkoxy is OCF₃, a linear O-linked hydrocarbon of between 1 and 4 carbon atoms (C₁-C₄), or a branched O-linked hydrocarbon of between 3 and 4 carbon atoms (C₃-C₄); small alkoxy may optionally be substituted with 1 or 2 substituents independently selected from cyclopropyl, OH, CN, CF₃, and halo;
aryl is phenyl, biphenyl or naphthyl; aryl may be optionally substituted with 1, 2 or 3 substituents independently selected from alkyl, alkoxy, methylenedioxy, ethylenedioxy, OH, halo, CN, —(CH₂)₀₋₃—NR₁₂R₁₃, OCF₃ and CF_{3;}
polycycloalkyl is a bicyclic saturated hydrocarbon ring system of between 5 and 8 carbon atoms (C₅-C₈), wherein the bicyclic ring is bridged, or is adamantyl; polycycloalkyl may optionally be substituted with 1 or 2 substituents independently selected from alkyl, (C₁-C₅) alkoxy, OH, CN, CF₃, and halo;
cycloalkyl is a monocyclic saturated hydrocarbon ring of between 3 and 6 carbon atoms (C₃-C₆); cycloalkyl may optionally be substituted with 1 or 2 substituents independently selected from alkyl, (C₁-C₆) alkoxy, OH, CN, CF₃, and halo;
halo is F, Cl, Br, or I;
heteroaryl is a 5- or 6-membered carbon-containing aromatic ring containing one, two or three ring members that are selected from N, S, and O; wherein heteroaryl may be optionally substituted with 1, 2 or 3 substituents independently selected from alkyl, alkoxy, cyclopropyl, OH, halo, CN, and CF_{3;}
heteroaryl^{a} is a 5-, 6-, 9- or 10-membered mono- or bi-cyclic aromatic ring containing, where possible, 1, 2, 3 or 4 ring members independently selected from N, S, and O; wherein heteroaryl^{a} may be optionally substituted with 1, 2 or 3 substituents independently selected from alkyl, alkoxy, cyclopropyl, OH, halo, CN, and CF_{3;}
heterocycloalkyl is a non-aromatic carbon-containing mono- or bi-cyclic ring containing 3, 4, 5, 6, or 7 ring members, wherein at least one ring member is independently selected from N, S, and O; heterocycloalkyl may optionally be substituted with 1 or 2 substituents independently selected from alkyl, cyclopropyl, (C₁-C₆) alkoxy, OH, CN, CF₃, oxo, and halo; wherein when heterocycloalkyl is a bi-cyclic ring system, the bicyclic ring system is fused, bridged or spiro;
each R₁₂ and R₁₃ is independently selected from H, alkyl, cycloalkyl, and heterocycloalkyl;
and tautomers, isomers, stereoisomers (including enantiomers, diastereoisomers and racemic and scalemic mixtures thereof), deuterated analogues, and pharmaceutically acceptable salts and/or solvates thereof.

2. A compound according to numbered aspect 1, or a tautomer, isomer, stereoisomer (including an enantiomer, a diastereoisomer and a racemic and scalemic mixture thereof), a deuterated analogue, and a pharmaceutically acceptable salt and/or solvate thereof, wherein:
(i) B₂ and B₃ together form a single bond between B₁ and the nitrogen atom to which R₁ is attached, or
(ii) B₂ is a carbon centre or a nitrogen centre and B₃ is a single bond between B₂ and the nitrogen atom to which R₁ is attached.

3. A compound according to any of the preceding numbered aspects, or a tautomer, isomer, stereoisomer (including an enantiomer, a diastereoisomer and a racemic and scalemic mixture thereof), a deuterated analogue, and a pharmaceutically acceptable salt and/or solvate thereof, wherein:
B₁ is a carbon centre, and B₂ and B₃ together form a single bond between B₁ and the nitrogen atom to which R₁ is attached,
(ii) B₁ is a carbon centre and B₂ is a carbon centre, and B₁ is connected to B₂ via a single bond, or

(iii) B₁ is a nitrogen centre and B₂ is a carbon centre and B₁ is connected to B₂ via a double bond.

4. A compound according to numbered aspect 3, or a tautomer, isomer, stereoisomer (including an enantiomer, a diastereoisomer and a racemic and scalemic mixture thereof), a deuterated analogue, and a pharmaceutically salt acceptable and/or solvate thereof, wherein B₁ is a carbon centre and B₂ is a carbon centre, and B₁ is connected to B₂ via a single bond, or wherein B₁ is a nitrogen centre and B₂ is a carbon centre and B₁ is connected to B₂ via a double bond.

5. A compound according to any of the preceding numbered aspects, or a tautomer, isomer, stereoisomer (including an enantiomer, a diastereoisomer and a racemic and scalemic mixture thereof), a deuterated analogue, and a pharmaceutically acceptable salt and/or solvate thereof, wherein:
(i) B₄ is N(R₂), B₅ is N, and B₆ is C(R₃), and B₄ is connected to B₅ via a single bond and B₅ is connected to B₆ via a double bond; or
(ii) B₄ is C(R₂), B₅ is N, and B₆ is N(R₃), and wherein B₄ is connected to B₅ via a double bond and B₅ is connected to B₆ via a single bond.

6. A compound according to any of the preceding numbered aspects, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₂ is selected from —C(═O)—N(R₄)(R₅), —C(═O)—O—R₆, —CH₂—NH—S(═O)₂(cyclopropyl), —CH₂—O—CH₃, —CH₂—OH, and —CN.

7. A compound according to any of the preceding numbered aspects, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₂ is selected from —C(═O)—N(R₄)(R₅), —C(═O)—O—R₆, —CH₂—NH—S(═O)₂(cyclopropyl), —CH₂—O—CH₃, and —CH₂—OH.

8. A compound according to any of the preceding numbered aspects, or a tautomer, isomer, stereoisomer (including an enantiomer, a diastereoisomer and a racemic and scalemic mixture thereof), a deuterated analogue, and a pharmaceutically acceptable salt and/or solvate thereof, wherein:
R₄ is H or small alkyl, and
R₅ is selected from alkyl, —(CH₂)₀₋₃-cycloalkyl, —(CH₂)₀₋₃-heterocycloalkyl, —(CH(alkyl))-heterocycloalkyl, —(CH₂)₀₋₃-polycycloalkyl, —(CH₂)₀₋₃-aryl, —(C(H)(phenyl))-alkyl, —(CH₂)₀₋₃heteroaryl^{a}, and —(C(H)(heteroaryl))-alkyl;
or wherein R₄ and R₅ together with the nitrogen atom to which they are attached form a carbon-containing 5-, or 6-membered saturated heterocyclic ring, optionally containing an additional heteroatom selected from N, and O, which may be optionally mono- or di-substituted with substituents selected from oxo, alkyl, alkoxy, OH, halo and CF₃; and
R₆, where present, is selected from H, alkyl, —(CH₂)₀₋₃-cycloalkyl, —(CH₂)₀₋₃-heterocycloalkyl, —(CH(alkyl)-heterocycloalkyl, —(CH₂)₀₋₃-polycycloalkyl, —(CH₂)₀₋₃-aryl, —(C(H)(phenyl)-alkyl, —(CH₂)₀₋₃heteroaryl, and —(C(H)(heteroaryl)-alkyl.

9. A compound according to any of the preceding numbered aspects, or a tautomer, isomer, stereoisomer (including an enantiomer, a diastereoisomer and a racemic and scalemic mixture thereof), a deuterated analogue, and a pharmaceutically acceptable salt and/or solvate thereof, wherein X₁ is selected from phenyl and pyridyl.

10. A compound according to any of the preceding numbered aspects, or a tautomer, isomer, stereoisomer (including an enantiomer, a diastereoisomer and a racemic and scalemic mixture thereof), a deuterated analogue, and a pharmaceutically acceptable salt and/or solvate thereof, wherein Formula (II) is represented by Formula (IIa),

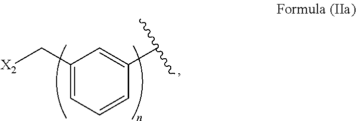

wherein:
n is 0 or 1;
X₂ is a non-aromatic, carbon-containing mono- or bi-cyclic ring containing 5, 6, or 7 ring members, wherein at least one ring member is independently selected from N, S, and O; wherein X₂ may be optionally substituted with 1 or 2 substituents R₁₈ and R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O.

11. A compound according to any of the preceding numbered aspects, or a tautomer, isomer, stereoisomer (including an enantiomer, a diastereoisomer and a racemic and scalemic mixture thereof), a deuterated analogue, and a pharmaceutically acceptable salt and/or solvate thereof, wherein X₂ is a non-aromatic carbon-containing mono- or bi-cyclic ring containing 5 or 6 ring members, wherein at least one ring member is N, and wherein, when X₂ is a bi-cyclic ring system, the bicyclic ring system is fused.

12. A compound according to any of the preceding numbered aspects, or a tautomer, isomer, stereoisomer (including an enantiomer, a diastereoisomer and a racemic and scalemic mixture thereof), a deuterated analogue, and a pharmaceutically acceptable salt and/or solvate thereof, wherein A is phenyl or a 6-membered carbon-containing aromatic ring containing one, two or three nitrogen ring members.

13. A compound according to any of the preceding numbered aspects, or a tautomer, isomer, stereoisomer (including an enantiomer, a diastereoisomer and a racemic and scalemic mixture thereof), a deuterated analogue, and a pharmaceutically acceptable salt and/or solvate thereof, wherein A′ is a non-aromatic carbon-containing monocyclic ring containing 5, 6, or 7 ring members, wherein at least one ring member is N.

14. A compound according to any of the preceding numbered aspects, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₇, when present, is bonded to A′ via a N ring member.

15. A compound according to any of the preceding numbered aspects, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₇ is (C₁-C₂)alkyl.

16. A compound according to any of the preceding numbered aspects, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₃ is not substituted with a 4-methoxy group.

17. A compound according to any of the preceding numbered aspects, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₃ is phenyl substituted with one or two substituents independently selected from small alkyl, small alkoxy, CF₃, halo, —C(═O)—NH₂, and CN.

18. A compound according to any of the preceding numbered aspects, or a tautomer, isomer, wherein R₃ is phenyl substituted with one or two halo substituents.

19. A compound according to any of the preceding numbered aspects, or a tautomer, isomer, stereoisomer (including an enantiomer, a diastereoisomer and a racemic and scalemic mixture thereof), a deuterated analogue, and a pharmaceutically acceptable salt and/or solvate thereof,
wherein R₃ is

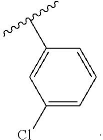 [SMILES: *c1cccc(Cl)c1]

20. A compound selected from Table 1a, Table 1b, Table 2a, Table 2b, Table 3a, Table 3b and Table 4, or a pharmaceutically acceptable salt, solvate, or solvate of a salt thereof.

21. A pharmaceutical composition comprising: a compound, or a pharmaceutically acceptable salt and/or solvate thereof, according to any of the preceding numbered aspects, and at least one pharmaceutically acceptable excipient.

22. A compound, or a pharmaceutically acceptable salt and/or solvate thereof, as defined in any of numbered aspects 1 to 20, or the pharmaceutical composition according to numbered aspect 21, for use in medicine.

23. A compound, or a pharmaceutically acceptable salt and/or solvate thereof, as defined in any of numbered aspects 1 to 20, or the pharmaceutical composition according to numbered aspect 21, for use in a method of treatment of a disease or condition in which Factor XIIa activity is implicated; wherein the disease or condition in which Factor XIIa activity is implicated is a bradykinin-mediated angioedema; wherein the bradykinin-mediated angioedema is hereditary angioedema.

24. A compound, or a pharmaceutically acceptable salt and/or solvate thereof, as defined in any of numbered aspects 1 to 20, or the pharmaceutical composition according to numbered aspect 21, for use in a method of treatment of a disease or condition in which Factor XIIa activity is implicated; wherein the disease or condition in which Factor XIIa activity is implicated is a bradykinin-mediated angioedema; wherein the bradykinin-mediated angioedema is non hereditary.

25. A compound, or a pharmaceutically acceptable salt and/or solvate thereof, as defined in any of numbered aspects 1 to 20, or the pharmaceutical composition according to numbered aspect 21, for use in a method of treatment of a disease or condition in which Factor XIIa activity is implicated; wherein the disease or condition in which Factor XIIa activity is implicated is a thrombotic disorder.

### Numbered Clauses

1. A compound of Formula (I),

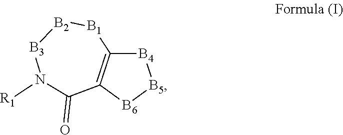

wherein:
B₁ is a carbon centre or nitrogen centre; and either:
(i) B₂ and B₃ together form a single bond between B₁ and the nitrogen atom to which R₁ is attached,
(ii) B₂ is a carbon centre or a nitrogen centre, and B₃ is a single bond between B₂ and the nitrogen atom to which R₁ is attached,
(iii) B₂ is a carbon centre and B₃ is a nitrogen centre, or
(iv) B₂ and B₃ are both carbon centres,
wherein, when B₂ is a carbon centre or nitrogen centre, B₁ is connected to B₂ via either a single or double bond;
wherein, when both B₂ and B₃ are present, B₂ and B₃ are connected via either a single or double bond;
wherein B₁ may, where possible, be optionally substituted with 1 or 2 substituents selected from alkyl, cycloalkyl, alkoxy, OH, CF₃, CN, and halo, or 1 oxo substituent, or 2 substituents which, together with B₁ to which they are both attached, form a 3-, 4-, or 5-membered non-aromatic monocyclic carbon-containing ring, optionally wherein one or two ring members are selected from N, S, and O;
wherein, when B₂ is a carbon centre, B₂ may be optionally substituted with 1 or 2 substituents selected from alkyl, cycloalkyl, alkoxy, OH, CF₃, CN, and halo, or 1 oxo substituent; and
wherein, when B₃ is a carbon centre, B₃ may be optionally substituted with 1 or 2 substituents selected from alkyl, cycloalkyl, alkoxy, OH, CF₃, CN, and halo, or 1 oxo substituent;
and either:
(i) B₄ is N(R₂), B₅ is N, and B₆ is C(R₃), and B₄ is connected to B₅ via a single bond and B₅ is connected to B₆ via a double bond;
(ii) B₄ is C(R₂), B₅ is N, and B₆ is N(R₃), and B₄ is connected to B₅ via a double bond and B₅ is connected to B₆ via a single bond; or
(iii) B₄ is C(R₂), B₅ is C(H), and B₆ is N(R₃), and B₄ is connected to B₅ via a double bond and B₅ is connected to B₆ via a single bond;
R₂ is selected from H, —(CH₂)₀₋₃—C(═O)—N(R₄)(R₅), —C(═O)—O—R₆, —(CH₂)₀₋₃—NR₁₄—C(═O)R₁₅, alkyl, cycloalkyl, heterocycloalkyl, alkoxy, —OH, —CF₃, halo, aryl, heteroaryl, —CH₂—O—R₁₆ and —CN;
wherein R₄ is H, alkyl, cycloalkyl, and heterocycloalkyl, and
R₅ is selected from alkyl, —(CH₂)₀₋₃-cycloalkyl, —(CH₂)₀₋₃-heterocycloalkyl, —(CH₂)₀₋₃-polycycloalkyl, —(CH₂)₀₋₃-aryl, —(C(H)(phenyl))-alkyl, —(CH₂)₀₋₃heteroaryl^{a}, and —(C(H)(heteroaryl))-alkyl;
or wherein R₄ and R₅ together with the nitrogen atom to which they are attached form a carbon-containing 4-, 5-, 6-, or 7-membered heterocyclic ring which may be saturated or unsaturated with 1 or 2 double bonds, optionally containing one or two additional heteroatoms selected from N, O, and S, wherein the ring may be optionally substituted with 1, 2, or 3 substituents selected from oxo, alkyl, alkoxy, OH, halo and CF_{3;}
R₆ is selected from H, alkyl, —(CH₂)₀₋₃-cycloalkyl, —(CH₂)₀₋₃-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, and —(C(H)(heteroaryl))-alkyl;
R₁₄ is selected from H, alkyl, cycloalkyl, and heterocycloalkyl;
R₁₅ is selected from H, alkyl, —(CH₂)₀₋₃-cycloalkyl, —(CH₂)₀₋₃-heterocycloalkyl, —(CH₂)₀₋₃-aryl, —(C(H)(phenyl)-alkyl, —(CH₂)₀₋₃heteroaryl^{a}, and —(C(H)(heteroaryl)-alkyl;
R₁₆ is selected from H, alkyl, cycloalkyl, and heterocycloalkyl;
R₁ is:
(i) Formula (II),

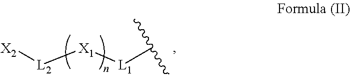

wherein:
n is 0 or 1;
L₁ is a linker selected from a single bond, —(CH₂)₁₋₃—, —(CH)(alkyl)-, and —C(alkyl)(alkyl)-;
X₁ is phenyl or a 5- or 6-membered carbon-containing aromatic ring containing one, two or three ring members that are selected from N, S, and O;
wherein X₁ may be optionally substituted with 1 or 2 substituents independently selected from alkyl, cycloalkyl, alkoxy, OH, CF₃, and halo;
L₂ is a linker selected from a single bond, —(CH₂)₁₋₃—, —(CH)(alkyl)-, —C(alkyl)(alkyl)-, —C(═O)—, and —SO₂—; and X₂ is either:
(a) —NR₂₀R₂₁,
wherein R₂₀ is selected from H, alkyl, and —(CH₂)₀₋₃—R_{22;}
wherein R₂₁ is selected from H, alkyl, and —(CH₂)₀₋₃—R₂₃;
wherein R₂₂ is selected from cycloalkyl, heterocycloalkyl, aryl, heteroaryl,

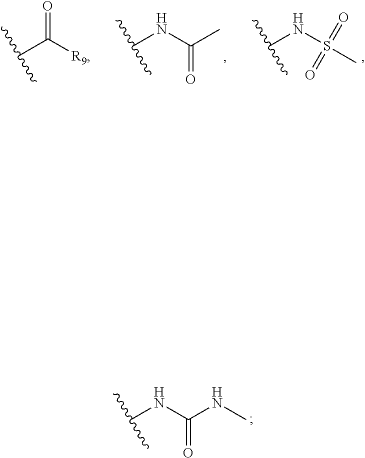

wherein R₂₃ is selected from cycloalkyl, heterocycloalkyl, aryl, heteroaryl,

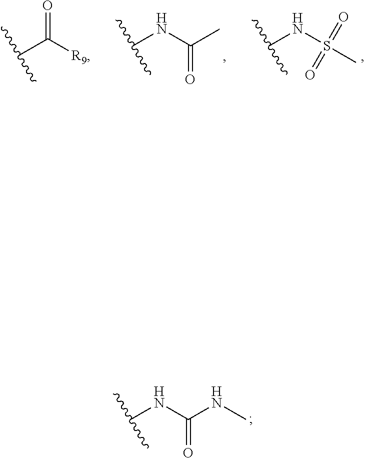

or
(b) 2-pyridone, 4-pyridone, or a non-aromatic, carbon-containing mono- or bi-cyclic ring containing 5, 6, or 7 ring members, wherein at least one ring member is independently selected from N, S, and O, which may be saturated or unsaturated with 1 or 2 double bonds which may be optionally substituted with 1 or 2 substituents R₁₈ and R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O; or,
(ii) Formula (III) comprising an aromatic ring A fused to a non-aromatic ring A′,

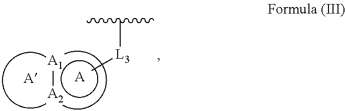

wherein:
L₃ is a linker selected from a single bond, —(CH₂)₁₋₃—, —(CH)(alkyl)-, and —C(alkyl)(alkyl)-;
A₁ and A₂ are both carbon;
A is phenyl or a 5- or 6-membered carbon-containing aromatic ring containing one, two or three ring members that are selected from N, S, and O;
wherein A may be optionally substituted with 1 or 2 substituents independently selected from alkyl, cycloalkyl, alkoxy, OH, CF₃, and halo;
A′ is a non-aromatic carbon-containing monocyclic ring containing 5, 6, or 7, ring members, wherein at least one ring member is independently selected from N, S, and O; wherein A′ may be optionally substituted with one R₇, wherein R₇ is selected from alkyl, alkoxy, and —(CH₂)₀₋₃—R₈,
wherein R₅ is selected from cycloalkyl, heterocycloalkyl, aryl, heteroaryl,

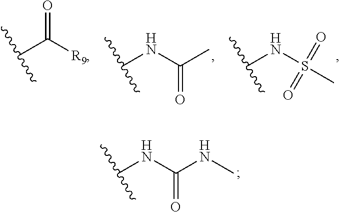

wherein R₉ is selected from H, alkyl, OH, NH₂, and alkoxy;
wherein A′ may be optionally substituted with 1 or 2 further substituents R₁₀ and R₁₁,
wherein R₁₀ and R₁₁ are independently selected from alkyl, cycloalkyl, alkoxy, OH, oxo, CF₃, and halo,
or wherein A′ may be optionally substituted with 1 further substituent R₂₄ selected from alkyl, cycloalkyl, alkoxy, OH, oxo, CF₃, and halo and 2 further substituents R₂₅ and R₂₆,
wherein R₂₅ and R₂₆ together with a carbon atom to which they are both attached form a 3-, 4-, or 5-membered non-aromatic monocyclic carbon-containing ring, optionally
wherein one or two ring members are selected from N, S, and O; and
R₃ is phenyl, or a 5- or 6-membered carbon-containing aromatic ring containing one, two or three ring members that are selected from N, S, and O, wherein R₃ is substituted with one or two substituents independently selected from small alkyl, small alkoxy, cyclopropyl, OH, CF₃, halo, —C(═O)—NH₂, —C(═O)—N(H)(small alkyl), —C(═O)—N(small alkyl)(small alkyl), and CN; and
alkyl is a linear saturated hydrocarbon having up to 10 carbon atoms (C₁-C₁₀) or a branched saturated hydrocarbon of between 3 and 10 carbon atoms (C₃-C₁₀); alkyl may optionally be substituted with 1, 2 or 3 substituents independently selected from cyclopropyl, (C₁-C₆) alkoxy, OH, —NR₁₂R₁₃, —C(═O) NR₁₂R₁₃, CN, CF₃, and halo;
small alkyl is a linear saturated hydrocarbon having up to 4 carbon atoms (C₁-C₄) or a branched saturated hydrocarbon of between 3 and 4 carbon atoms (C₃-C₄); small alkyl may optionally be substituted with 1 or 2 substituents independently selected from cyclopropyl, OH, CN, CF₃, NH₂, —C(═O)NH₂, and halo;
alkoxy is OCF₃, a linear O-linked hydrocarbon of between 1 and 6 carbon atoms (C₁-C₆), or a branched O-linked hydrocarbon of between 3 and 6 carbon atoms (C₃-C₆); alkoxy may optionally be substituted with 1 or 2 substituents independently selected from cyclopropyl, OH, CN, CF₃, and halo;
small alkoxy is OCF₃, a linear O-linked hydrocarbon of between 1 and 4 carbon atoms (C₁-C₄), or a branched O-linked hydrocarbon of between 3 and 4 carbon atoms (C₃-C₄); small alkoxy may optionally be substituted with 1 or 2 substituents independently selected from cyclopropyl, OH, CN, CF₃, and halo;
aryl is phenyl, biphenyl or naphthyl; aryl may be optionally substituted with 1, 2 or 3 substituents independently selected from alkyl, alkoxy, methylenedioxy, ethylenedioxy, OH, halo, CN, —(CH₂)₀₋₃—NR₁₂R₁₃, OCF₃ and CF_{3;}
polycycloalkyl is a bicyclic saturated hydrocarbon ring system of between 5 and 8 carbon atoms (C₅-C₈), wherein the bicyclic ring is bridged, or is adamantyl; polycycloalkyl may optionally be substituted with 1 or 2 substituents independently selected from alkyl, (C₁-C₅) alkoxy, OH, CN, CF₃, and halo;
cycloalkyl is a monocyclic saturated hydrocarbon ring of between 3 and 6 carbon atoms (C₃-C₆); cycloalkyl may optionally be substituted with 1 or 2 substituents independently selected from
halo is F, Cl, Br, or I;
heteroaryl is a 5- or 6-membered carbon-containing aromatic ring containing one, two or three ring members that are selected from N, S, and O; wherein heteroaryl may be optionally substituted with 1, 2 or 3 substituents independently selected from alkyl, alkoxy, OH, halo, CN, and CF_{3;}
heteroaryl^{a} is a 5-, 6-, 9- or 10-membered mono- or bi-cyclic aromatic ring containing, where possible, 1, 2, 3 or 4 ring members independently selected from N, S, and O; wherein heteroaryl^{a} may be optionally substituted with 1, 2 or 3 substituents independently selected from alkyl, alkoxy, OH, halo, CN, and CF_{3;}
heterocycloalkyl is a non-aromatic carbon-containing mono- or bi-cyclic ring containing 3, 4, 5, 6, or 7 ring members, wherein at least one ring member is independently selected from N, S, and O; heterocycloalkyl may optionally be substituted with 1 or 2 substituents independently selected from alkyl, cyclopropyl, (C₁-C₆) alkoxy, OH, CN, CF₃, oxo, and halo; wherein when heterocycloalkyl is a bi-cyclic ring system, the bicyclic ring system is fused, bridged or spiro;
each R₁₂ and R₁₃ is independently selected from H, alkyl, cycloalkyl, and heterocycloalkyl;
and tautomers, isomers, stereoisomers (including enantiomers, diastereoisomers and racemic and scalemic mixtures thereof), deuterated analogues, and pharmaceutically acceptable salts and/or solvates thereof.

2. A compound according to numbered clause 1, or a tautomer, isomer, stereoisomer (including an enantiomer, a diastereoisomer and a racemic and scalemic mixture thereof), a deuterated analogue, and a pharmaceutically salt acceptable and/or solvate thereof, wherein B₂ is a carbon centre or a nitrogen centre and B₃ is a single bond between B₂ and the nitrogen atom to which R₁ is attached.

3. A compound according to any of the preceding numbered clauses, or a tautomer, isomer, stereoisomer (including an enantiomer, a diastereoisomer and a racemic and scalemic mixture thereof), a deuterated analogue, and a pharmaceutically acceptable salt and/or solvate thereof, wherein B₁ is a carbon centre and B₂ is a carbon centre, and B₁ is connected to B₂ via a single bond, or wherein B₁ is a nitrogen centre and B₂ is a carbon centre and B₁ is connected to B₂ via a double bond.

4. A compound according to numbered clause 3, or a tautomer, isomer, stereoisomer (including an enantiomer, a diastereoisomer and a racemic and scalemic mixture thereof), a deuterated analogue, and a pharmaceutically acceptable salt and/or solvate thereof, wherein B₁ is a carbon centre and B₂ is a carbon centre, and B₁ is connected to B₂ via a single bond.

5. A compound according to any of the preceding numbered clauses, or a tautomer, isomer, stereoisomer (including an enantiomer, a diastereoisomer and a racemic and scalemic mixture thereof), a deuterated analogue, and a pharmaceutically acceptable salt and/or solvate thereof, wherein B₄ is C(R₂), B₅ is N, and B₆ is N(R₃), and wherein B₄ is connected to B₅ via a double bond and B₅ is connected to B₆ via a single bond.

6. A compound according to any of the preceding numbered clauses, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₂ is selected from —C(═O)—N(R₄)(R₅), —C(═O)—O—R₆, —CH₂—O—CH₃, and —CN.

7. A compound according to any of the preceding numbered clauses, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₂ is selected from —C(═O)—N(R₄)(R₅), —C(═O)—O—R₆, and —CH₂—O—CH_{3.}

8. A compound according to any of the preceding numbered clauses, or a tautomer, isomer, stereoisomer (including an enantiomer, a diastereoisomer and a racemic and scalemic mixture thereof), a deuterated analogue, and a pharmaceutically acceptable salt and/or solvate thereof, wherein:
R₄ is H or small alkyl, and
R₅ is selected from alkyl, —(CH₂)₀₋₃-cycloalkyl, —(CH₂)₀₋₃-heterocycloalkyl, —(CH₂)₀₋₃-polycycloalkyl, —(CH₂)₀₋₃-aryl, —(C(H)(phenyl))-alkyl, —(CH₂)₀₋₃heteroaryl^{a}, and —(C(H)(heteroaryl))-alkyl;
or wherein R₄ and R₅ together with the nitrogen atom to which they are attached form a carbon-containing 5-, or 6-membered saturated heterocyclic ring, optionally containing an additional heteroatom selected from N, and O, which may be optionally mono- or di-substituted with substituents selected from oxo, alkyl, alkoxy, OH, halo and CF₃; and
R₆, where present, is selected from H, alkyl, —(CH₂)₀₋₃-cycloalkyl, —(CH₂)₀₋₃-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, and —(C(H)(heteroaryl))-alkyl.

9. A compound according to any of the preceding numbered clauses, or a tautomer, isomer, stereoisomer (including an enantiomer, a diastereoisomer and a racemic and scalemic mixture thereof), a deuterated analogue, and a pharmaceutically acceptable salt and/or solvate thereof, wherein X₁ is selected from phenyl and pyridyl.

10. A compound according to any of the preceding numbered clauses, or a tautomer, isomer, stereoisomer (including an enantiomer, a diastereoisomer and a racemic and scalemic mixture thereof), a deuterated analogue, and a pharmaceutically acceptable salt and/or solvate thereof, wherein Formula (II) is represented by Formula (IIa),

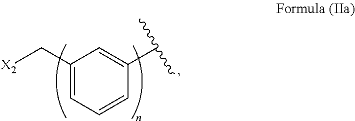

wherein:
n is 0 or 1;
X₂ is a non-aromatic, carbon-containing mono- or bi-cyclic ring containing 5, 6, or 7 ring members, wherein at least one ring member is independently selected from N, S, and O; wherein X₂ may be optionally substituted with 1 or 2 substituents R₁₈ and R₁₉, wherein R₁₈ and R₁₉ are independently selected from alkyl, cycloalkyl, alkoxy, OH, oxo, CF₃, and halo, or wherein R₁₈ and R₁₉, where possible, together with a carbon atom to which they are both attached form a 3-, 4-, or 5-membered non-aromatic monocyclic carbon-containing ring, optionally wherein one or two ring members are selected from N, S, and O.

11. A compound according to any of the preceding numbered clauses, or a tautomer, isomer, wherein X₂ is a non-aromatic carbon-containing mono- or bi-cyclic ring containing 5 or 6 ring members, wherein at least one ring member is N, and wherein, when X₂ is a bi-cyclic ring system, the bicyclic ring system is fused.

12. A compound according to any of the preceding numbered clauses, or a tautomer, isomer, stereoisomer (including an enantiomer, a diastereoisomer and a racemic and scalemic mixture thereof), a deuterated analogue, and a pharmaceutically acceptable salt and/or solvate thereof, wherein n is 1.

13. A compound according to any of the preceding numbered clauses, or a tautomer, isomer, stereoisomer (including an enantiomer, a diastereoisomer and a racemic and scalemic mixture thereof), a deuterated analogue, and a pharmaceutically acceptable salt and/or solvate thereof, wherein A is phenyl or a 6-membered carbon-containing aromatic ring containing one, two or three nitrogen ring members.

14. A compound according to any of the preceding numbered clauses, or a tautomer, isomer, stereoisomer (including an enantiomer, a diastereoisomer and a racemic and scalemic mixture thereof), a deuterated analogue, and a pharmaceutically acceptable salt and/or solvate thereof, wherein A′ is a non-aromatic carbon-containing monocyclic ring containing 5, 6, or 7 ring members, wherein at least one ring member is N.

15. A compound according to any of the preceding numbered clauses, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₇, when present, is bonded to A′ via a N ring member.

16. A compound according to any of the preceding numbered clauses, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₇ is (C₁-C₂)alkyl.

17. A compound according to any of the preceding numbered clauses, or a tautomer, isomer, wherein R₃ is not substituted with a 4-methoxy group.

18. A compound according to any of the preceding numbered clauses, or a tautomer, isomer, stereoisomer (including an enantiomer, a diastereoisomer and a racemic and scalemic mixture thereof), a deuterated analogue, and a pharmaceutically acceptable salt and/or solvate thereof, wherein R₃ is phenyl substituted with one or two halo substituents.

19. A compound according to any of the preceding numbered clauses, or a tautomer, isomer, stereoisomer (including an enantiomer, a diastereoisomer and a racemic and scalemic mixture thereof), a deuterated analogue, and a pharmaceutically acceptable salt and/or solvate thereof,
wherein R₃ is

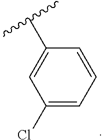 [SMILES: *c1cccc(Cl)c1]

20. A compound selected from Table 1a, Table 2a, and Table 3a, or a pharmaceutically acceptable salt, solvate, or solvate of a salt thereof.

21. A pharmaceutical composition comprising: a compound, or a pharmaceutically acceptable salt and/or solvate thereof, according to any of the preceding numbered clauses, and at least one pharmaceutically acceptable excipient.

22. A compound, or a pharmaceutically acceptable salt and/or solvate thereof, as defined in any of numbered clauses 1 to 20, or the pharmaceutical composition according to numbered clause 21, for use in medicine.

23. A compound, or a pharmaceutically acceptable salt and/or solvate thereof, as defined in any of numbered clauses 1 to 20, or the pharmaceutical composition according to numbered clause 21, for use in a method of treatment of a disease or condition in which Factor XIIa activity is implicated; wherein the disease or condition in which Factor XIIa activity is implicated is a bradykinin-mediated angioedema; wherein the bradykinin-mediated angioedema is hereditary angioedema.

24. A compound, or a pharmaceutically acceptable salt and/or solvate thereof, as defined in any of numbered clauses 1 to 20, or the pharmaceutical composition according to numbered clause 21, for use in a method of treatment of a disease or condition in which Factor XIIa activity is implicated; wherein the disease or condition in which Factor XIIa activity is implicated is a bradykinin-mediated angioedema; wherein the bradykinin-mediated angioedema is non hereditary.

25. A compound, or a pharmaceutically acceptable salt and/or solvate thereof, as defined in any of numbered clauses 1 to 20, or the pharmaceutical composition according to numbered clause 21, for use in a method of treatment of a disease or condition in which Factor XIIa activity is implicated; wherein the disease or condition in which Factor XIIa activity is implicated is a thrombotic disorder.

### Synthetic Methods

The compounds of the present invention can be prepared according to the procedures of the following schemes and examples, using appropriate materials, and are further exemplified by the specific examples provided herein below. Moreover, by utilising the procedures described herein, one of ordinary skill in the art can readily prepare additional compounds that fall within the scope of the present invention claimed herein. The compounds illustrated in the examples are not, however, to be construed as forming the only genus that is considered as the invention. Those skilled in the art will readily understand that known variations of the conditions, processes, and order in which the synthetic steps are performed in the following preparative procedures can be used to prepare these compounds.

The compounds and intermediates of the invention may be isolated in the form of their pharmaceutically acceptable salts, such as those described previously herein above. The interconversion between free form and salt form would be readily known to those skilled in the art.

It may be necessary to protect reactive functional groups (e.g. hydroxy, amino, thio or carboxy) in intermediates used in the preparation of compounds of the invention to avoid their unwanted participation in a reaction leading to the formation of the compounds. Conventional protecting groups, for example those described by Peter G. M. Wuts in “Greene's Protective Groups in Organic Chemistry” John Wiley and Sons, 5^{th} Edition, 2014., may be used. For example, a common amino protecting group suitable for use herein is tert-butoxy carbonyl (boc), which is readily removed by treatment with an acid such as trifluoroacetic acid or hydrogen chloride in an organic solvent such as dichloromethane. Alternatively, the amino protecting group may be a benzyloxycarbonyl (Cbz or Z) group which can be removed by hydrogenation with a palladium catalyst under a hydrogen atmosphere or 9-fluorenylmethyloxycarbonyl (Fmoc) group which can be removed by solutions of secondary organic amines such as diethylamine or piperidine in an organic solvent. Carboxyl groups are typically protected as esters such as methyl, ethyl, benzyl or tert-butyl which can all be removed by hydrolysis in the presence of bases such as lithium or sodium hydroxide. Benzyl protecting groups can also be removed by hydrogenation with a palladium catalyst under a hydrogen atmosphere whilst tert-butyl groups can also be removed by trifluoroacetic acid. Alternatively, a trichloroethyl ester protecting group is removed with zinc in acetic acid. A common hydroxy protecting group suitable for use herein is a methyl ether, deprotection conditions comprise refluxing in 48% aqueous HBr, or by stirring with borane tribromide in an organic solvent such as DCM. Alternatively, where a hydroxy group is protected as a benzyl ether, deprotection conditions comprise hydrogenation with a palladium catalyst under a hydrogen atmosphere.

The Ullmann coupling chemistry described herein refers to copper-catalysed nucleophilic aromatic substitution between various nucleophiles (e.g, substituted phenoxides, amines, amides) with aryl halides.

Palladium catalysed cross coupling reactions to form carbon-carbon, carbon-nitrogen and carbon-oxygen bonds typically require a palladium catalyst and ligand capable of forming an in situ catalytically active LₙPd⁽⁰⁾ species.

The graphic representations of racemic, ambiscalemic and scalemic or enantiomerically pure compounds used herein are taken from Maehr J. Chem. Ed. 62, 114-120 (1985): solid wedges ( [symbol] ) and broken wedges ( [symbol] ) are used to denote the absolute configuration of a chiral element; wavy lines ( [symbol] ) indicate disavowal of any stereochemical implication which the bond it represents could generate; solid bold lines ( [symbol] ) and broken bold lines ( [symbol] ) are geometric descriptors indicating the relative configuration shown, but denoting racemic character; and wedge outlines ( [symbol] ) and broken lines ( [symbol] ) denote enantiomerically pure compounds of indeterminate absolute configuration. For nomenclature in the text corresponding to wedge outlines ( [symbol] ) and broken lines ( [symbol] ) we define R* and S* as indicating single enantiomers of uncertain absolute configuration.

The invention is illustrated by the following non-limiting examples in which the following abbreviations and definitions are used:

| | |
|---|---|
| AcOH | acetic acid |
| aq | aqueous solution |
| AIBN | azobisisobutyronitrile |
| BFMO | N,N′-difurfuryloxamide |
| boc | tert-butoxy carbonyl |
| Boc₂O | di-tert-butyl dicarbonate |
| B₂Pin₂ | bis(pinacolato)diboron |
| BrettPhos | 2-(dicyclohexylphosphino)-3,6-dimethoxy-2′,4′,6′-triisopropyl-1,1′- |
| | biphenyl |
| BrettPhos Pd G3 | [(2-di-cyclohexylphosphino-3,6-dimethoxy-2′,4′,6′-triisopropyl-1,1′- |
| | biphenyl)-2-(2′-amino-1,1′-biphenyl)]palladium(II) methanesulfonate |
| BrettPhos Pd G4 | methanesulfonato(2-dicyclohexylphosphino-3,6-dimethoxy-2′,4′,6′-tri-i- |
| | propyl-1,1′-biphenyl)(2′-methylamino-1,1′-biphenyl-2-yl)palladium(II) |
| | (CAS no. 1599466-83-7) |
| tBu | tert-butyl |
| tert-BuBrettphos Pd G3 | [(2-di-tert-butylphosphino-3,6-dimethoxy-2′,4′,6′-triisopropyl-1,1′- |
| | biphenyl)-2-(2′-amino-1,1′-biphenyl)]palladium(II) methanesulfonate |
| nBuLi | n-butyllithium |
| Cbz | benzyl carbamate |
| CDI | 1,1′-carbonyldiimidazole |
| Celite ® | Filter agent (diatomaceous earth) |
| COMU | 1-cyano-2-ethoxy-2-oxoethylidenaminooxy)dimethylamino-morpholino- |
| | carbenium hexafluorophosphate |
| DBU | 1,8-diazabicyclo[5.4.0]undec-7-ene |
| DCE | 1,2-dichloroethane |
| DCM | dichloromethane |
| DEA | diethylamine |
| Dess-Martin | 1,1,1-tris(acetyloxy)-1,1-dihydro-1,2-benziodoxol-3-(1H)-one |
| periodinane | |
| DIAD | diisopropyl azodicarboxylate |
| DIPEA | N,N-diisopropylethylamine |
| DMAP | 4-dimethylaminopyridine |
| DMF | N,N-dimethylformamide |
| DMSO | dimethyl sulfoxide |
| EDC | 1-ethyl-3-(3-dimethylaminopropyl)carbodiimide hydrochloride |
| eq | equivalent |
| Et₂O | diethyl ether |
| Et | ethyl |
| EtOH | ethanol |
| EtOAc | ethyl acetate |
| HATU | 2-(3H-[1,2,3]triazolo[4,5-b]pyridin-3-yl)-1,1,3,3-tetramethylisouronium |
| | hexafluorophosphate(V) |
| HBTU | N,N,N′,N′-tetramethyl-O-(1H-benzotriazol-1-yl)uronium |
| | hexafluorophosphate |
| h | hours |
| HOBt | 1-hydroxybenzotriazole hydrate |
| IPA | isopropyl alcohol |
| KHMDS | potassium hexamethyldisilazide |
| LCMS | liquid chromatography mass spectrometry |
| LiHMDS | lithium hexamethyldisilazide |
| Me | methyl |
| MeCN | acetonitrile |
| MeI | iodomethane |
| MeOH | methanol |
| min | minutes |
| MS | mass spectrum |
| Ms | methanesulfonyl |
| MsCl | methanesulfonyl chloride |
| NaHMDS | sodium hexamethyldisilazide |
| NBS | N-bromosuccinimide |
| NCS | N-chlorosuccinimide |
| NMR | nuclear magnetic resonance spectrum |
| NMP | N-methyl-2-pyrrolidone |
| OAc | acetate |
| Pd-175 | [^{t}BuBrettPhos Pd(allyl)]OTf, allyl(2-di-tert-butylphosphino-3,6- |
| | dimethoxy-2′,4′,6′-triisopropyl-1,1′-biphenyl)palladium(II) triflate |
| Pd(dppf)Cl₂:DCM | [1,1′-bis(diphenylphosphino)ferrocene]dichloropalladium(II), complex |
| | with dichloromethane |
| Pd-PEPPSI-IPent | [1,3-bis(2,6-di-3-pentylphenyl)imidazol-2-ylidene](3- |
| | chloropyridyl)palladium(II) dichloride |
| Pd₂(dba)₃ | tris(dibenzylideneacetone)dipalladium(0) |
| Pet. Ether | petroleum ether fraction boiling at 60-80° C. |
| Ph | phenyl |
| Pin | pinacol |
| PyBOP | benzotriazole-1-yl-oxy-tris-pyrrolidino-phosphonium |
| | hexafluorophosphate |
| iPr | iso-propyl |
| nPr | n-propyl |
| RockPhos Pd G3 | [(2-di-tert-butylphosphino-3-methoxy-6-methyl-2′,4′,6′-triisopropyl-1,1′- |
| | biphenyl)-2-(2-aminobiphenyl)]palladium(II) methanesulfonate |
| rt | room temperature |
| RuPhos Pd G3 | (2-dicyclohexylphosphino-2′,6′-diisopropoxy-1,1′-biphenyl)[2-(2′-amino- |
| | 1,1′-biphenyl)]palladium(II) methanesulfonate |
| RuPhos Pd G4 | methanesulfonato(2-dicyclohexylphosphino-2′,6′-di-i-propoxy-1,1′- |
| | biphenyl)(2′-methylamino-1,1′-biphenyl-2-yl)palladium(II) |
| RuPhos | 2-dicyclohexylphosphino-2′,6′-diisopropoxybiphenyl |
| sat. | saturated |
| SCX | strong cation exchange cartridge |
| SEM-Cl | 2-(trimethylsilyl)ethoxymethyl chloride |
| SFC | supercritical fluid chromatography |
| STAB | sodium triacetoxyborohydride |
| SWFI | sterile water for injection |
| T3P | propanephosphonic acid anhydride |
| TBAB | tetra-n-butylammonium bromide |
| TBAF | tetra-n-butylammonium fluoride |
| TDAI | tetra-n-butylammonium iodide |
| TBDMS | tert-butyldimethylsilyl |
| TBME | tert-butyl methyl ether |
| TBDPS | tert-butyldiphenylsilyl |
| TCFH | chloro-N,N,N′,N′-tetramethylformamidinium hexafluorophosphate |
| THF | tetrahydrofuran |
| TEA | triethylamine |
| TFA | trifluoroacetic acid |
| XantPhos | 4,5-bis(diphenylphosphino)-9,9-dimethylxanthene |
| XantPhos Pd G4 | palladium, [[5-(diphenylphosphino)-9,9-dimethyl-9H-xanthen-4- |
| | yl]diphenylphosphine-κP](methanesulfonato-κO)[2′-(methylamino- |
| | κN)[1,1′-biphenyl]-2-yl-κC]-, (SP-4-3)- |
| Z | benzyl carbamate |

All reactions were carried out under an atmosphere of nitrogen unless specified otherwise.

Hydrogenations were typically carried out using an H-Cube® reactor (manufactured by Thalesnano, Inc, Hungary).

References to the use of microwave, a microwave reactor, microwave heating and microwave irradiation all refer to the use of a CEM Discover Microwave Reactor.

¹H NMR spectra were recorded on a Bruker (500 MHz or 400 MHz) spectrometer with a Bruker Avance II or Avance Ill console or on a Jeol 400 MHz and are reported as chemical shift (ppm). It will be understood that, where exchangeable protons are present in any compound, the number of protons in the ¹H NMR spectra may not exactly correspond to the number of protons in the structure of any compound synthesised herein.

Molecular ions were obtained using LCMS with appropriate conditions selected from:
Waters Acquity H-Class LC system equipped with a Waters Acquity QDa mass spectrometer with electrospray ionisation (ESI). Waters Acquity UPLC BEH C18 2.1×50 mm 1.7 μm column. Mobile phase A:-0.1% formic acid in CH₃CN, mobile phase B:-0.1% formic acid in water. Flow rate 1.0 mL/min, gradient 2-98% B over 3 min.
UPLC-MS analysis was carried out on a Waters Acquity UPLC system consisting of an Acquity I-Class Sample Manager-FL, Acquity I-Class Binary Solvent Manager and an Acquity UPLC Column Manager. UV detection was afforded using an Acquity UPLC PDA detector (scanning from 210 to 400 nm), whilst mass detection was achieved using an Acquity QDa detector (mass scanning from 100-1250 Da; positive and negative modes simultaneously), and ELS detection was achieved using an Acquity UPLC ELS Detector. Waters Acquity UPLC BEH C18 column (2.1×50 mm, 1.7 μm). Typically Mobile Phase A:-0.1% v/v formic acid in water; mobile phase B 0.1% v/v formic acid in MeCN; flow rate 0.8 mL/min; gradient 5-95% B over 4 min. It will be understood that variations of this method can used as required.

It will be understood that alternative conditions (such as choice of column, flow rate ranges, solvents, modifiers and pH) may be used for LCMS in order to separate and obtain mass spectra.

Flash chromatography was typically carried out over ‘silica’ (silica gel for chromatography, 0.035 to 0.070 mm (220 to 440 mesh)(e.g. Merck silica gel 60)), and an applied pressure of nitrogen up to 10 p.s.i accelerated column elution. Alternatively, pre-prepared cartridges of silica gel were used, for example pre-packed SiliaSep™ columns from Silicycle or Sfär C18 D-Duo 100 Å 30 μm columns from Biotage. Typical conditions included, for example, flow rate range of 18-200 ml/min, with an applied pressure range of 0-225 PSI. Hexanes, EtOAc, DCM, MeOH, were used as mobile phases for normal-phase chromatography purifications. MeOH, MeCN, 10 mM Ammonium Formate pH 4 in H₂O and 10 mM Ammonium Bicarbonate pH 10 in H₂O buffers were used as mobile phases for reverse-phase chromatography. It will be understood that alternative conditions (such as flow rate ranges, applied pressures, solvents and pH) may be used for flash chromatography in order to separate and purify compounds synthesised herein.

The term “prep HPLC” refers to reverse phase preparative HPLC purifications. Preparative HPLC was carried out using a Waters AutoPurification HPLC/MS system equipped with a Waters Acquity QDa mass detector with electrospray ionisation (ESI). Typical conditions were Waters SunFire Prep C18 OBD 19×250 mm 5 μm column. Mobile phase A:-0.1% formic acid in water, mobile phase B:-0.1% formic acid in MeCN. Flow rate 20 ml/min, gradient 2-98% B over 20 min. It will be understood that alternative conditions (such as choice of column, flow rate ranges, solvents, modifiers and pH) may be used for prep HPLC in order to separate and purify compounds synthesised herein.

The procedure of lyophilisation (or freeze drying) is generally well known in the art. Typically the substance is taken up in water, if necessary, with the addition of a minimum amount of MeCN to aid dissolution, and frozen, typically by rapid cooling in a cold bath at −78° C. The resulting frozen solid mixture is evaporated to dryness in vacuo.

The terms “concentrated” and “evaporated” refer to evaporation of solvent under reduced pressure using a rotary evaporator, heating where necessary.
All solvents and commercial reagents were used as received.

IUPAC chemical names were generated using automated software such as Dotmatics Studies Notebook or ChemDraw (PerkinElmer). Compounds produced by the methods below may be isolated in salt forms. However, compound naming used herein typically refers to the compound without any salt counter ion.

The example compounds described herein can be prepared using conventional synthetic methods for example, but not limited to, the routes outlined in the General Schemes below.

### General Schemes

General Schemes 1-5 outline synthetic routes for certain example compounds and RgA, RgB, RgC, RgD and RgE refer to various substituents as required by the examples. Availability of starting materials and reactivity/sensitivity of RgA, RgB, RgC, RgD and RgE may dictate the route chosen by those skilled in the art.

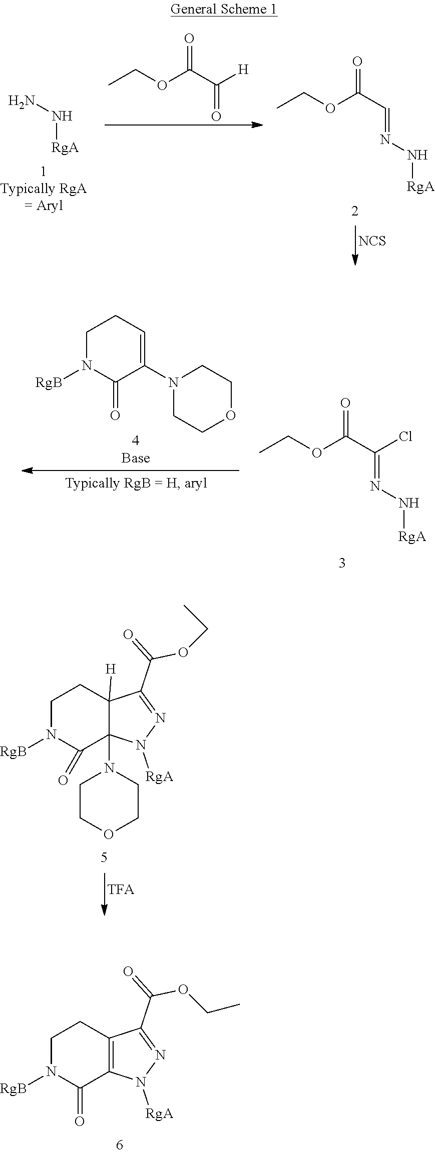

The chemistry in General Scheme 1 has been published in WO2003049681A₂. Hydrazines such as 1, where RgA=aryl, are reacted with a glycolate ester, exemplified in this case by ethyl glycolate, typically in THF at 0-25° C., where the ethyl group is in the role of a protecting group. Alternative protecting groups can be used with different deprotection conditions such that an orthogonal protecting group strategy can be applied. The procedures for different protecting groups are described for example by Peter G. M. Wuts in “Greene's Protective Groups in Organic Chemistry” John Wiley and Sons, 5^{th} Edition, 2014. The resulting hydrazone 2 is reacted with a chlorinating agent such as NCS, typically at rt in solvents such as THF, to give the chlorohydrazone 3. Treatment with a base such as TEA at 0° C. in EtOAc, or K₂CO₃ in dioxane at 85° C. produces the nitrile imine enabling a cycloaddition with a dipolarophile such as 4 (RgB=H, aryl) to afford the intermediate 5. The desired 4.5-dihydro-pyrazolo[3,4-c]pyrid-2-one derivative 6 can be generated by elimination of morpholine typically using TFA in solvents such as DCM. Derivatives 6 can be used as key intermediates where, as in the specific examples of the invention described later, RgB is tetrahydroisoquinoline and there follows elaboration of the carboxylate functionality to the desired compounds.
Alternatively, where further elaboration of the RgB group is required, as well as elaboration of the carboxylate functionality, this can be achieved via the chemistries described in General Scheme 2 following either Path A or Path B as required by the specific examples. General Scheme 2 illustrates the use of a Boc protecting group. Alternative protecting groups may be used. The protected intermediate 9 is obtained from, for example, an Ullmann coupling (e.g. Cul, K₂CO₃, 1,10-phenanthroline, DMSO, 130° C.) of the 4.5-dihydro-pyrazolo[3,4-c]pyrid-2-one derivative 7 with Boc protected tetrahydroisoquinoline derivative 8. Alternatively, a palladium catalysed cross coupling reaction may be used for this step. Examples of palladium catalysed cross couplings are Buchwald couplings. A Buchwald coupling is carried out for example using BrettPhos Pd G3, BrettPhos Pd G4 or RuPhos Pd G3 catalyst in the presence of a base such a NaOtBu, Cs₂CO₃, or KHMDS, in a solvent such as 1,4-dioxane or THF. In Path B, base hydrolysis of the ethyl ester 9 to carboxylic acid 14 is typically carried out using LiOH in water/THF at rt. Amide formation with RgC to give 15 and deprotection of the Boc group to give 16 can be carried out using a variety of conditions, all generally known in the art, for example amide coupling with HBTU/TEA in DCM at rt and deprotection using HCl in 1,4-dioxane. Finally, reactions to form alkylated tetrahydroquinoline derivatives 16 can be achieved either by alkylation using a halide RgC-X or through a reductive amination procedure with an aldehyde or ketone. Typically, alkylation conditions with an alkyl halide Rg-X require a base such as TEA and are carried out in a polar solvent such as DMF. Reductive amination conditions utilise various borane reducing reagents to reduce the intermediate iminium species, for example STAB. Path A in Scheme 2 describes the same transformations as Path B except the sequence is different.

General Scheme 3 shows the synthesis of examples 19 where RgB is not tetrahydroisoquinoline and starting from the key intermediate 7. Intermediate 7 is exemplified with an ethyl ester where the ethyl group is in the role of a protecting group. Alternative protecting groups may be used as described for example by Peter G. M. Wuts in “Greene's Protective Groups in Organic Chemistry” John Wiley and Sons, 5th Edition, 2014. Path A describes an Ullmann coupling (typically Cul, K₂CO₃, 1,10-phenanthroline, DMSO, 130° C.) to give the ester 17 with the required-N(RgE)(RgD) moiety installed in one step. Alternatively, a palladium catalysed cross coupling reaction may be used for this step. Examples of palladium catalysed cross couplings are Buchwald couplings. A Buchwald coupling is carried out for example using BrettPhos Pd G3, BrettPhos Pd G4 or RuPhos Pd G3 catalyst in the presence of a base such a NaOtBu, Cs₂CO₃, or

KHMDS, in a solvent such as 1,4-dioxane or THF. Where, as exemplified in General Scheme 3, an ethyl protecting group is being used, base hydrolysis is used to remove the protecting group, typically LiOH in THF/water to give 18. If an alternative protecting group is being used then the appropriate deprotection conditions are described for example by Peter G. M. Wuts in “Greene's Protective Groups in Organic Chemistry” John Wiley and Sons, 5^{th} Edition, 2014. There follows amide formation with RgC-NH2 which can be carried out using a variety of coupling reactions such as with HBTU/TEA in DCM at rt.

Path B illustrates an orthogonal protecting group strategy and may be used to allow additional possibilities for the —N(RgE)(RgD) moiety as it is installed in the final step of the sequence. The first step involves an Ullmann coupling (typically Cul, K₂CO₃, 1,10-phenanthroline, DMSO, 130° C.) with a protected benzyl alcohol 26 to give ester 20. Alternatively, a palladium catalysed cross coupling may be used. Typically the protecting group is TBDMS but other protecting groups as described for example by Peter G. M. Wuts in “Greene's Protective Groups in Organic Chemistry” may be used. Where, as exemplified in General Scheme 3, the orthogonal protecting group is an ethyl ester, base hydrolysis is used to remove the protecting group, typically LiOH in THF/water to give 21. There follows amide formation with RgC-NH2 which can be carried out using a variety of coupling reactions such as with HBTU/TEA in DCM at rt to give 22. Removal of the protecting group PG gives the benzyl alcohol 23, for example where the protecting group is TBDMS this can be achieved with TBAF in THF. The alcohol 23 can be oxidised to aldehyde 24, for example using Dess-Martin periodinane or by using MnO2, thus allowing subsequent reductive amination with a range of amines HN (RgE)(RgD) and which may use a variety of borane reducing reagents, for example STAB, to reduce the intermediate iminium species to give 19. Alternatively, benzyl alcohol 23 can be converted to a suitable leaving group, for example a mesylate obtained using MsCl and base, and reacted with amines HN (RgE)(RgD) in an SN₂ reaction (typically using K₂CO₃ in DMF) again to give 19.

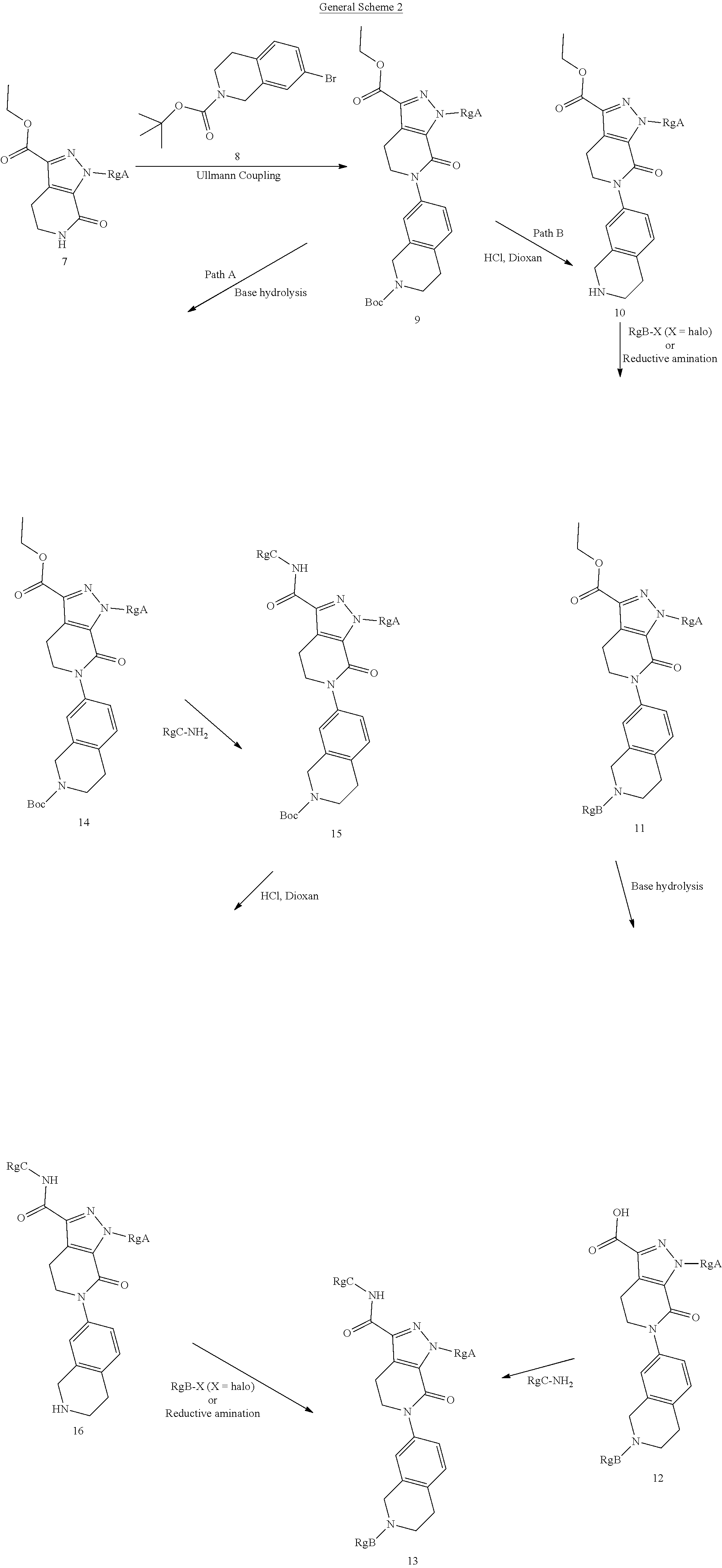

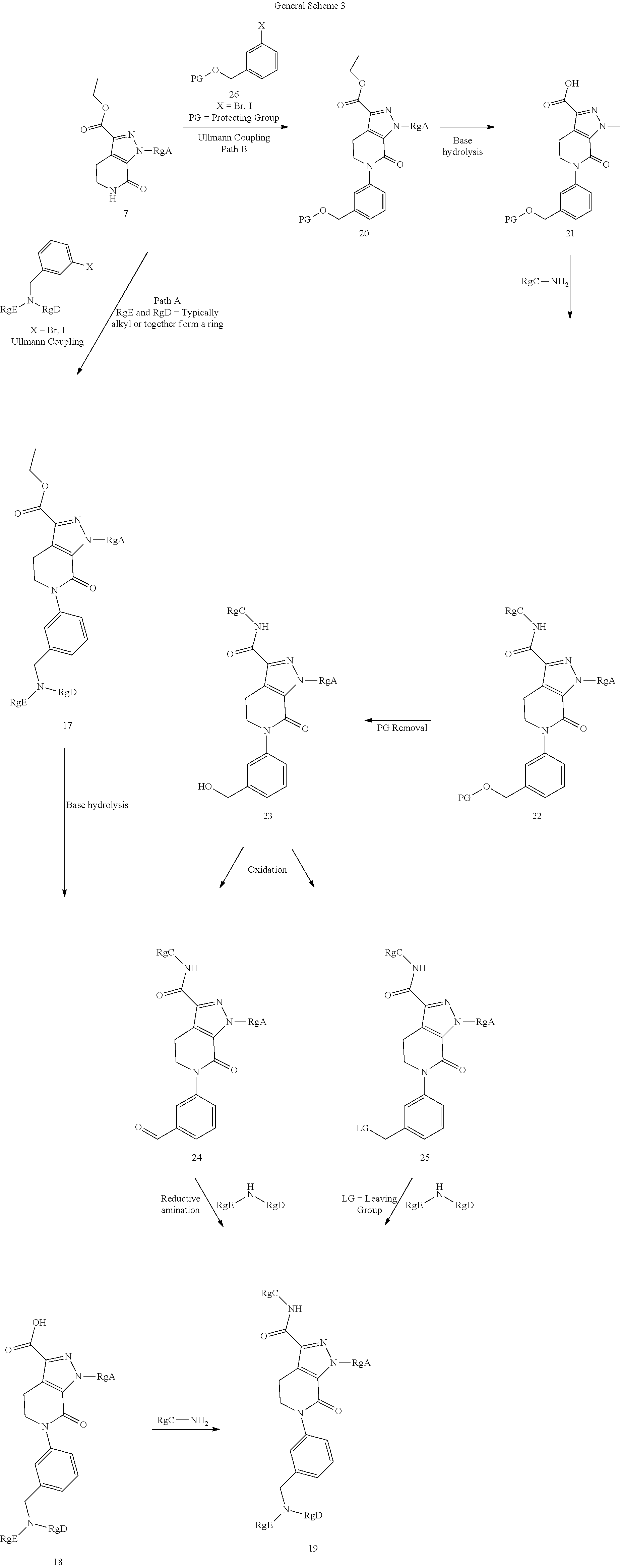

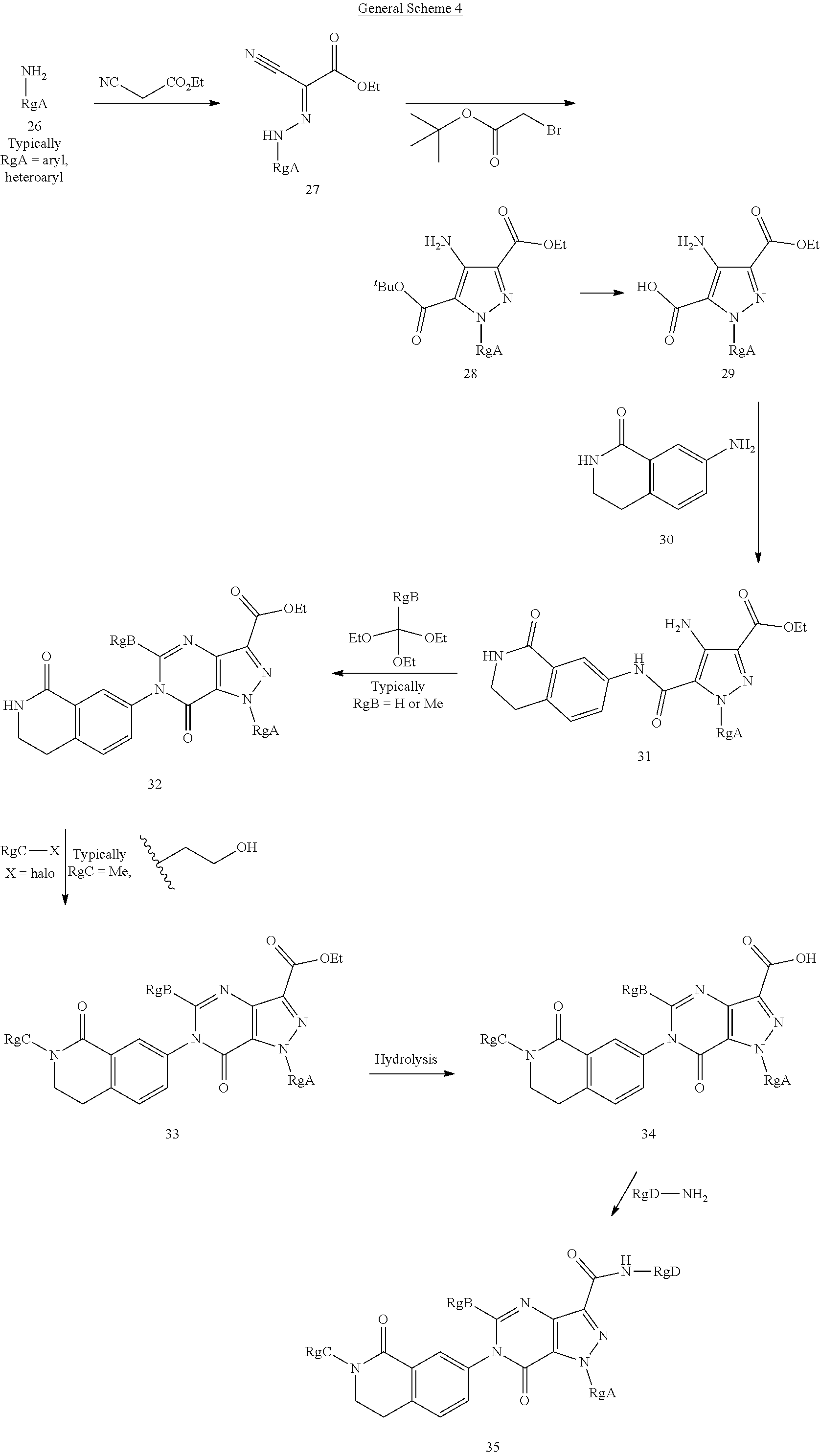

General Scheme 4 shows the synthetic scheme for the preparation of 7-oxo-pyrazolo[4,3-d]pyrimidines 5 exemplified in Tables 3a, 3b and 3c. Typically, in compound 26, RgA is aryl or heteroaryl and is diazotised using NaNO₂ in HCl (aq) and reacted with ethyl cyanoacetate and NaOAc to give the hydrazone intermediate 27. Alternatively (not shown in Scheme 4), malononitirile may be used in place of ethyl cyanoacetate to provide the nitrile analogue of the ester 27. Subsequent reaction with t-butyl bromoacetate and cyclisation gives the pyrazole intermediate 28, in which the t-butyl ester is selectively hydrolysed (HCl in dioxane at rt) to the intermediate 29. Coupling with 30 (for example using HATU and DIPEA at rt) allows attachment of the isoquinolinone fragment. Alternatively (not shown in Scheme 4) it is possible to couple a protected form isoquinoline (typically with Boc protection on nitrogen). Cyclisation with triethylorthoacetate (where RgB=Me) or triethylorthoformate (where RgB=H) gives the 7-oxo-pyrazolo[4,3-d]pyrimidine intermediate 32. Intermediate 32 can be alkylated at the amide position with RgC-X, (typical conditions are NaH, DMF, Mel at rt) to give derivatives 33. Following hydrolysis (for example with LiOH in water/THF at rt) to the carboxylic acid 34, the desired amide products 35 are formed with RgD-NH₂ which can be carried out using a variety of coupling reactions generally known in the art such as with HBTU/TEA in DCM at rt.

For some compounds of the invention, such as the examples shown in Tables 2a, 2b and 2c, it is useful to be able to add functionality at a later stage of the synthetic scheme to allow expedient synthesis of a wide range of derivatives. Hence Scheme 5 shows a variation of Scheme 1 whereby compound 41 can be used as a common intermediate to synthesise a range of aryl or heteroaryl 1-substituted 4,5-dihydro-pyrazolo[3,4-c]pyrid-2-ones. In this case 4-methoxybenzyl hydrazine is reacted with ethyl-2-oxoacetate to form the hydrazone 36, typically in THF at 0-25° C. and where the ethyl group is present as a protecting group. Alternative protecting groups can be used with different deprotection conditions such that an orthogonal protecting group strategy can be applied. The procedures for different protecting groups are described for example by Peter G. M. Wuts in “Greene's Protective Groups in Organic Chemistry” John Wiley and Sons, 5th Edition, 2014. Hydrazone 36 is reacted with a chlorinating agent such as NCS, typically at rt in solvents such as THF, to give the chlorohydrazone 37. Treatment with a base, such as TEA at 0° C. in EtOAc, or K₂CO₃ in dioxane at 85° C., produces a nitrile imine (not shown), enabling a cycloaddition with a dipolarophile such as 39 to afford the intermediate 40. The desired 4,5-dihydro-pyrazolo[3,4-c]pyrid-2-one common intermediate 41 can be generated by elimination of morpholine and concomitant removal of the 4-methoxybenzyl group, typically using TFA in solvents such as DCM. In compounds of the invention, as exemplified in Tables 2a, 2b and 2c, RgA may be aryl or heteroaryl and an Ullmann reaction (M. B. Smith, J. March “March's Advanced Organic Chemistry” 6^{th} Edition, 2007, Wiley-Blackwell, 897-898) or a Chan-Lam reaction (K. S. Rao, T. Wu, Chan-Lam coupling reactions: synthesis of heterocycles, Tetrahedron, Volume 68, Issue 38, 2012, 7735-7754, https://doi.org/10.1016/j.tet.2012.06.015.) may be used to provide 42. Subsequent hydrolysis to remove the ethyl protecting group (for example with LiOH in water/THF at rt) to give 43, and coupling with the appropriate amine, RgB-NH₂, (using conditions generally known in the art such as HBTU/TEA in DCM at rt) gives the desired amide compound 44.

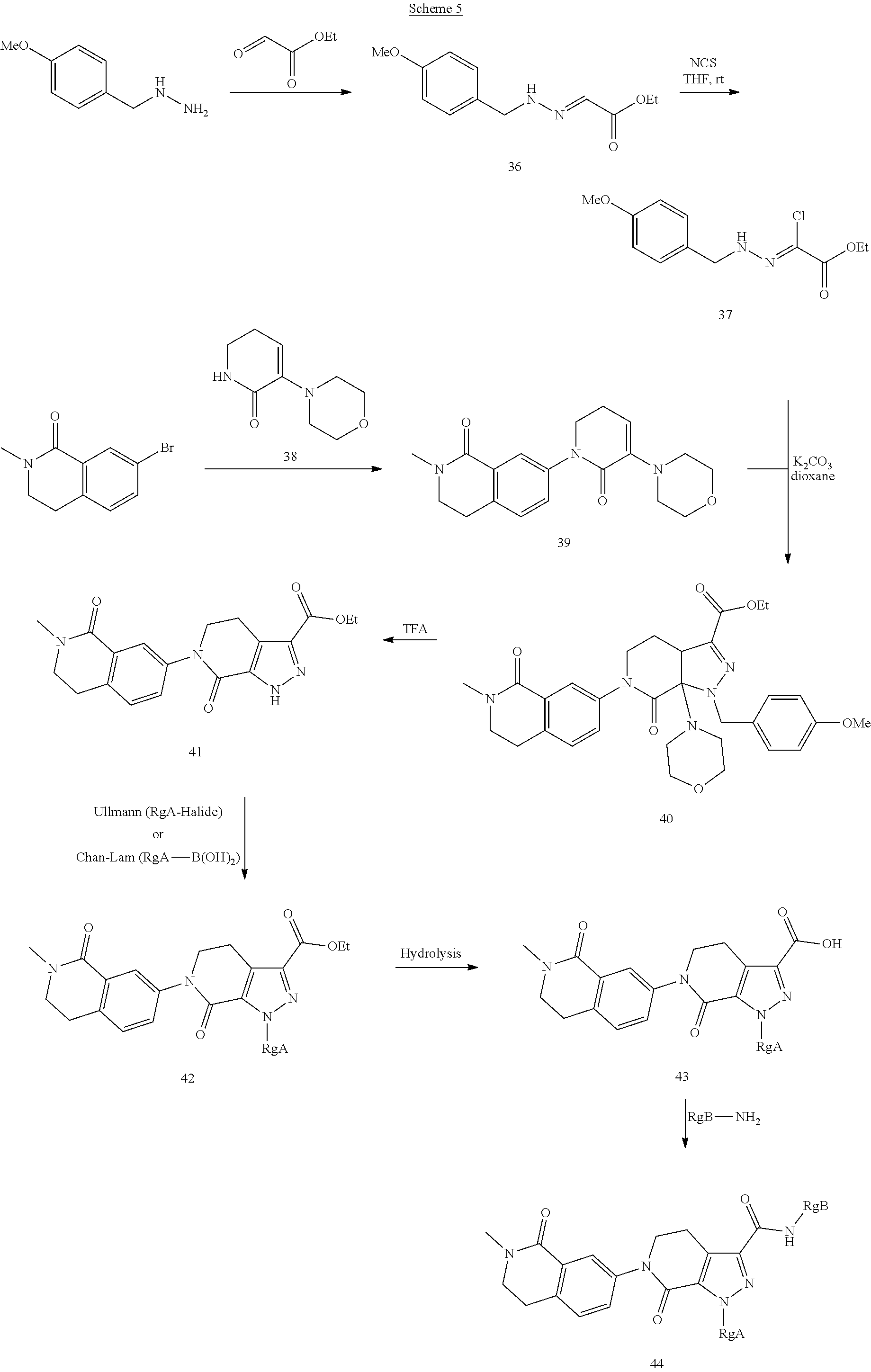

### SYNTHESIS OF INTERMEDIATES

### Intermediate 1

### Ethyl 1-(3-chlorophenyl)-7-oxo-5,6-dihydro-4H-pyrazolo[3,4-c]pyridine-3-carboxylate

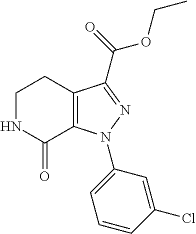 [SMILES: CCOC(=O)c1nn(-c2cccc(Cl)c2)c2c1CCNC2=O]

### Ethyl 2-[(3-chlorophenyl) hydrazinylidene] acetate

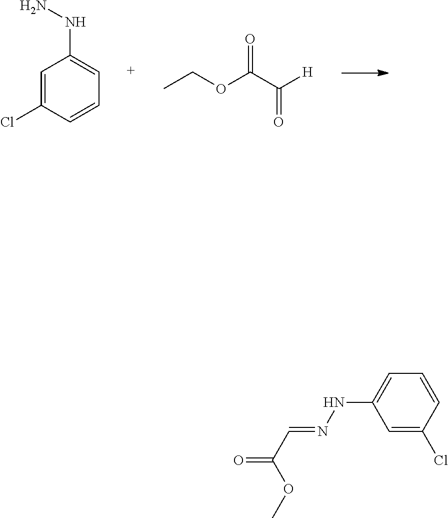

3-Chlorophenyl hydrazine hydrochloride (500 mg, 2.79 mmol) was dissolved THF (25 mL) and TEA (467 μL, 3.35 mmol) was added. Ethyl 2-oxoacetate (50% solution in toluene)(609 μL, 3.07 mmol) was added dropwise at 0° C. The mixture was stirred at rt for 7 days. The mixture was filtered and the filtrate evaporated. The residue was purified via flash chromatography (Silica, 0-2% MeOH in DCM) to afford the product (291 mg, 46% yield).

[M+H]⁺=227.0

### Ethyl 2-chloro-2-[(3-chlorophenyl) hydrazinylidene] acetate

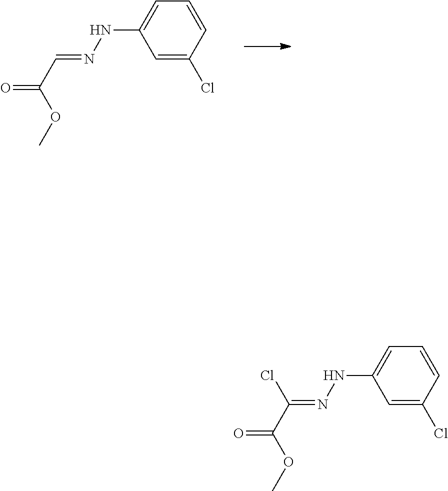

To a solution of ethyl 2-[(3-chlorophenyl) hydrazinylidene] acetate (291 mg, 1.28 mmol) in dry THF (20 mL) at 0° C. was added NCS (206 mg, 1.54 mmol) and the mixture was stirred at rt for 18 h. The solvent was evaporated and the residue was purified by flash chromatography (Silica, 0-50% EtOAc in Pet. Ether) to afford the product (331 mg, 99% yield).

[M+H]⁺=261.0

### Ethyl (3aS,7aR)-1-(3-chlorophenyl)-7a-morpholin-4-yl-7-oxo-3a,4,5,6-tetrahydropyrazolo[3,4-c]pyridine-3-carboxylate

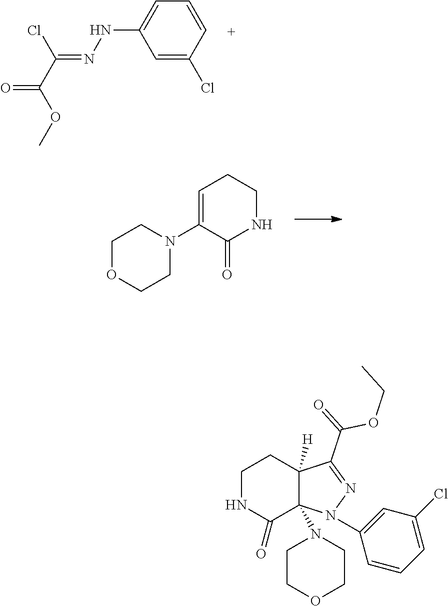 [SMILES: COC(=O)/C(Cl)=N/Nc1cccc(Cl)c1 ; O=C1NCCC=C1N1CCOCC1 ; CCOC(=O)C1=NN(c2cccc(Cl)c2)[C@@]2(N3CCOCC3)C(=O)NCC[C@@H]12]

Ethyl 2-chloro-2-[(3-chlorophenyl) hydrazinylidene] acetate (331 mg, 1.27 mmol) was dissolved in EtOAc (10 mL). The solution was cooled to 0° C. and 3-(morpholin-4-yl)-1,2,5,6-tetrahydropyridin-2-one (508 mg, 1.39 mmol) was added followed by TEA (442 μL, 3.17 mmol). The mixture was warmed to rt over 30 min and then heated at reflux for 18 h. The mixture was cooled to rt, diluted with EtOAc (10 mL) and washed with water (1×30 mL). The aqueous layer was extracted with EtOAc. Combined organic extracts were washed with brine (1×30 mL), dried (MgSO₄) and evaporated. Flash chromatography (Silica, 0-50% EtOAc in Pet. Ether) afforded the product as a racemic mixture (295 mg, 57% yield).

[M+H]⁺=407.1 1H NMR (CDCl₃, 400 MHZ) δ 1.40 (3H, t, J=7.1 Hz), 1.86-1.99 (1H, m), 2.33-2.47 (3H, m), 2.61 (2H, s), 3.06-3.20 (2H, m), 3.66-3.84 (4H, m), 4.01 (1H, d, J=6.5 Hz), 4.36 (2H, q, J=6.9 Hz), 6.90-7.02 (1H, m), 7.14-7.23 (1H, m), 7.81 (2H, d, J=1.4 Hz) NH proton was not observed

### Ethyl 1-(3-chlorophenyl)-7-oxo-5,6-dihydro-4H-pyrazolo[3,4-c]pyridine-3-carboxylate

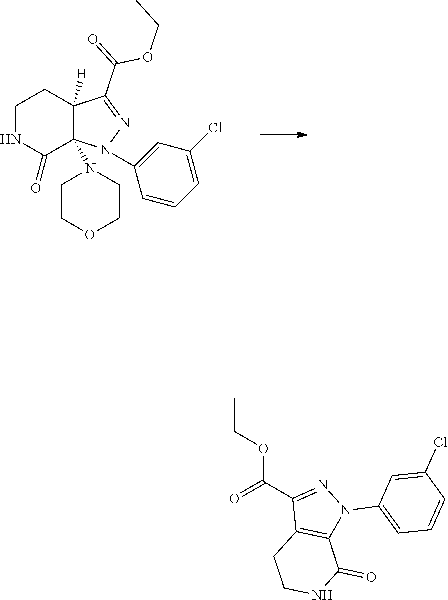 [SMILES: CCOC(=O)C1=NN(c2cccc(Cl)c2)[C@@]2(N3CCOCC3)C(=O)NCC[C@@H]12 ; CCOC(=O)c1nn(-c2cccc(Cl)c2)c2c1CCNC2=O]

Ethyl (3aS,7aR)-1-(3-chlorophenyl)-7a-morpholin-4-yl-7-oxo-3a,4,5,6-tetrahydropyrazolo[3,4-c]pyridine-3-carboxylate (as a racemic mixture)(295 mg, 0.73 mmol) was taken up in DCM (2 mL). TFA (500 μL) was added and the mixture was stirred at rt for 30 min. The mixture was evaporated and purified via flash chromatography (Silica, 0-100% EtOAc in Pet. Ether) to afford the product (143 mg, 62% yield).

[M+H]⁺=320.0

¹H NMR (CDCl₃, 400 MHZ) δ 1.43 (3H, t, J=7.1 Hz), 3.20 (2H, d, J=7.0 Hz), 3.65 (2H, td, J=6.8, 2.9 Hz), 4.45 (2H, d, J=7.1 Hz), 5.71 (1H, s), 7.31-7.58 (3H, m), 7.58-7.85 (1H, m)

### Intermediate 2

### tert-Butyl 7-[1-(3-chlorophenyl)-3-ethoxycarbonyl-7-oxo-4,5-dihydropyrazolo[3,4-c]pyridin-6-yl]-3,4-dihydro-1H-isoquinoline-2-carboxylate

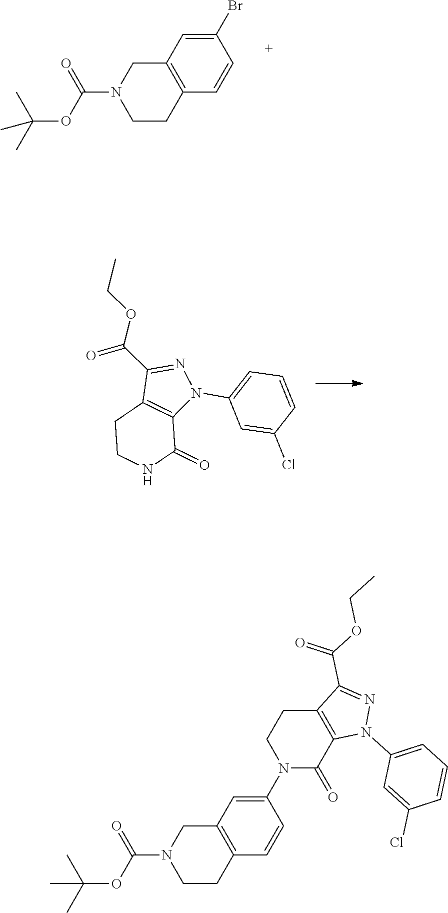 [SMILES: CC(C)(C)OC(=O)N1CCc2ccc(Br)cc2C1 ; CCOC(=O)c1nn(-c2cccc(Cl)c2)c2c1CCNC2=O ; CCOC(=O)c1nn(-c2cccc(Cl)c2)c2c1CCN(c1ccc3c(c1)CN(C(=O)OC(C)(C)C)CC3)C2=O]

Ethyl 1-(3-chlorophenyl)-4,5,6,7-tetrahydro-7-oxo-1H-pyrazolo[3,4-c]pyridine-3-carboxylate (1000 mg, 3.13 mmol) was dissolved in DMSO (3 mL), 1,10-phenanthroline (113 mg, 0.63 mmol), Cu(I)I (655 mg, 3.44 mmol), tert-Butyl 7-bromo-3,4-dihydroisoquinoline-2 (1H)-carboxylate (1074 mg, 3.44 mmol) and K₂CO₃ (865 mg, 6.26 mmol) were added. The reaction mixture was stirred at 130° C. for 1 h after which time it was diluted with EtOAc (70 mL). This solution was washed with water (3×30 mL), brine (1×30 mL), dried (Na₂SO₄) and evaporated. The residue was purified by flash chromatography (Silica, 1% MeOH in CHCl₃) to afford the product (1200 mg, 70% yield).

[M+H+Na]⁺=575.1

### Intermediate 3

### tert-Butyl7-[1-(3-chlorophenyl)-3-ethoxycarbonyl-7-oxo-4,5-dihydropyrazolo[3,4-c]pyridin-6-yl]-5-methoxy-3,4-dihydro-1H-isoquinoline-2-carboxylate

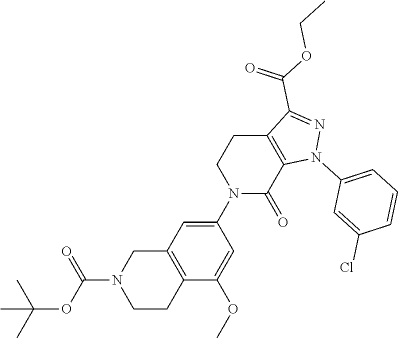 [SMILES: CCOC(=O)c1nn(-c2cccc(Cl)c2)c2c1CCN(c1cc3c(c(OC)c1)CCN(C(=O)OC(C)(C)C)C3)C2=O]

### N-[(3-Bromo-5-methoxy-phenyl)methyl]-2,2-dimethoxy-ethanamine

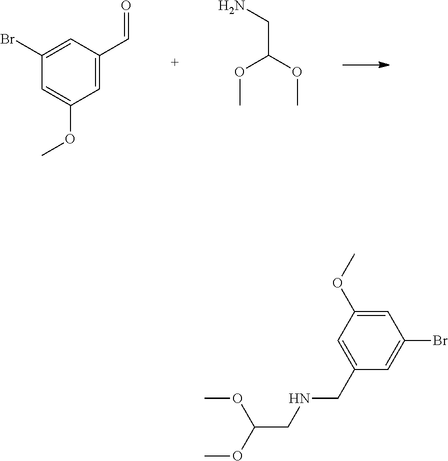

To a solution of 3-bromo-5-methoxybenzaldehyde (1000 mg, 4.65 mmol) in toluene (15 mL) was added aminoacetaldehyde dimethyl acetal (538 mg, 5.12 mmol) and the resultant mixture was heated at 120° C. for 18 h. On cooling, the mixture was concentrated, redissolved in MeOH (15 mL) and treated with NaBH4 (202 mg, 5.35 mmol) while cooling in an ice/water bath. After warming up to rt the reaction was left stirring for 3 h. The mixture was quenched with ice/water (20 mL) and extracted with EtOAc (3×50 mL). The combined organics were washed with brine (50 mL), dried (Na₂SO₄), filtered and concentrated to afford the product (1370 mg, 91% yield).

[M+H]⁺=304.1/306.1

¹H NMR (DMSO-d6, 400 MHZ) δ 2.49 (2H, d, J=5.5 Hz), 3.20 (6H, s), 3.62 (2H, s), 3.71 (3H, s), 4.35 (1H, t, J=5.5 Hz), 6.77-6.92 (1H, m), 6.95 (1H, dd, J=2.4, 1.8 Hz), 7.00-7.11 (1H, m)

### 7-Bromo-5-methoxy-1,2,3,4-tetrahydroisoquinolin-4-ol hydrochloride

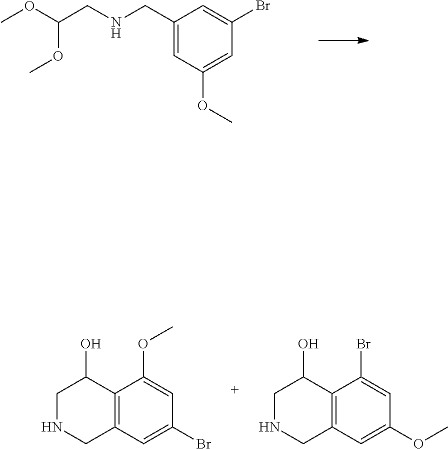 [SMILES: COc1cc(Br)cc(CNCC(OC)OC)c1 ; COc1cc(Br)cc2c1C(O)CNC2 ; COc1cc(Br)c2c(c1)CNCC2O]

A suspension of N-[(3-bromo-5-methoxy-phenyl)methyl]-2,2-dimethoxy-ethanamine (1370 mg, 4.23 mmol) in 6M HCl (aq)(3.0 mL, 18.0 mmol) was heated at 40° C. for 18 h. The mixture was concentrated to afford a mixture (1430 mg) of 7-bromo-5-methoxy-1,2,3,4-tetrahydroisoquinolin-4-ol hydrochloride and 5-bromo-7-methoxy-1,2,3,4-tetrahydroisoquinolin-4-ol hydrochloride as a solid which was used without purification in the next step.

Both product peaks on LCMS had [M+H]⁺=258.1/260.1 . . .

### 7-Bromo-5-methoxy-1,2,3,4-tetrahydroisoquinoline

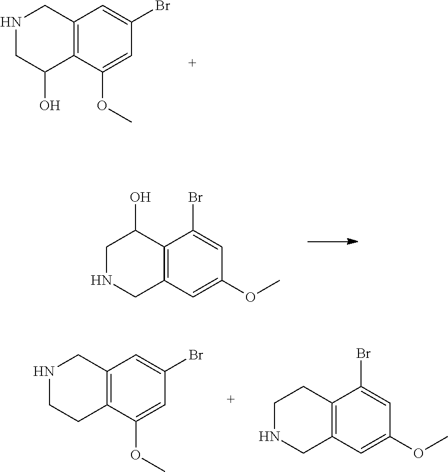

A mixture containing 7-bromo-5-methoxy-1,2,3,4-tetrahydroisoquinolin-4-ol and 5-bromo-7-methoxy-1,2,3,4-tetrahydroisoquinolin-4-ol (1230 mg) in DCM (6.6 mL) was treated with triethylsilane (1.5 mL, 9.53 mmol) and TFA (2.8 mL, 37.0 mmol). The mixture was heated at 40° C. for 18 h, concentrated and the residue taken up in EtOAc (100 mL). This was washed with sat. NaHCO₃(aq)(30 mL), brine (30 mL), dried (Na₂SO₄), filtered and concentrated to afford a mixture of 7-bromo-5-methoxy-1,2,3,4-tetrahydroisoquinoline and 5-bromo-7-methoxy-1,2,3,4-tetrahydroisoquinoline (2120 mg) which was used directly in the next step.

[M+H]⁺=242.1/244.0

### tert-Butyl 7-bromo-5-methoxy-3,4-dihydro-1H-isoquinoline-2-carboxylate

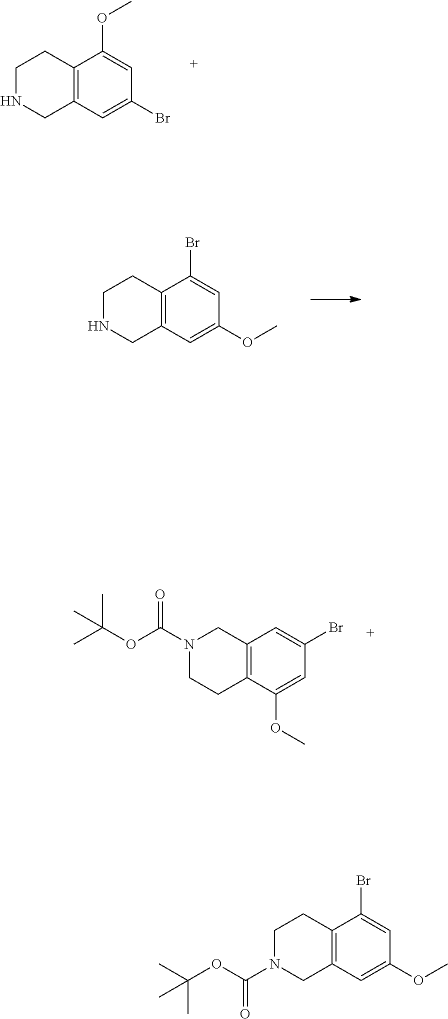 [SMILES: COc1cc(Br)cc2c1CCNC2 ; COc1cc(Br)c2c(c1)CNCC2 ; COc1cc(Br)cc2c1CCN(C(=O)OC(C)(C)C)C2 ; COc1cc(Br)c2c(c1)CN(C(=O)OC(C)(C)C)CC2]

A mixture of 7-bromo-5-methoxy-1,2,3,4-tetrahydroisoquinoline and 5-bromo-7-methoxy-1,2,3,4-tetrahydroisoquinoline (2014 mg) in DCM (12 mL) was treated with TEA (4.3 mL, 31.1 mmol) followed by Boc₂O (1604 mg, 7.35 mmol) and the resultant mixture was left stirring at rt for 18 h. The mixture was diluted with DCM (100 mL), washed with water (50 mL), brine (50 mL), dried (Na₂SO₄), filtered and concentrated. Flash chromatography (silica, 0-80% EtOAc in iso-hexane) afforded a mixture of tert-butyl 7-bromo-5-methoxy-3,4-dihydro-1H-isoquinoline-2-carboxylate and tert-butyl 5-bromo-7-methoxy-3,4-dihydro-1H-isoquinoline-2-carboxylate (280 mg) which was used directly in the next step.

[M+H−Boc]⁺=242.0/244.0

### tert-Butyl7-[1-(3-chlorophenyl)-3-ethoxycarbonyl-7-oxo-4,5-dihydropyrazolo[3,4-c]pyridin-6-yl]-5-methoxy-3,4-dihydro-1H-isoquinoline-2-carboxylate

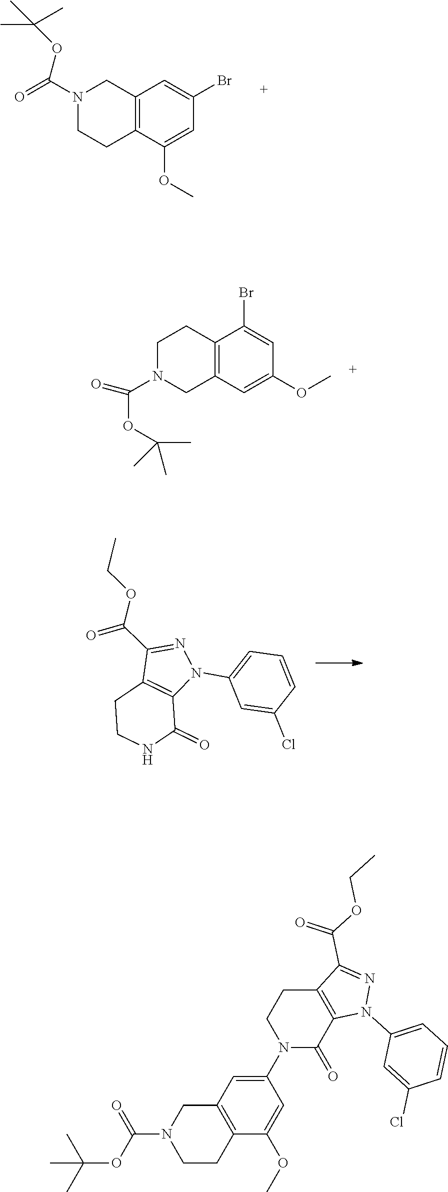 [SMILES: COc1cc(Br)cc2c1CCN(C(=O)OC(C)(C)C)C2 ; COc1cc(Br)c2c(c1)CN(C(=O)OC(C)(C)C)CC2 ; CCOC(=O)c1nn(-c2cccc(Cl)c2)c2c1CCNC2=O ; CCOC(=O)c1nn(-c2cccc(Cl)c2)c2c1CCN(c1cc3c(c(OC)c1)CCN(C(=O)OC(C)(C)C)C3)C2=O]

A mixture of tert-butyl 7-bromo-S-methoxy-3,4-dihydro-1H-isoquinoline-2-carboxylate and tert-butyl 5-bromo-7-methoxy-3,4-dihydro-1H-isoquinoline-2-carboxylate (244 mg, 0.392 mmol), and ethyl 1-(3-chlorophenyl)-7-oxo-5,6-dihydro-4H-pyrazolo[3,4-c]pyridine-3-carboxylate (190 mg, 0.594 mmol) were dissolved in 1,4-dioxane (6 ml). Cu(I)I (23 mg, 0.119 mmol), (1S,2S)—N,N′-dimethyl-1,2-cyclohexane-diamine (34 mg, 0.238 mmol) and tri-potassium phosphate (315 mg, 1.49 mmol) were added and the resultant mixture was stirred at 100° C. for 72 h. The mixture was diluted with EtOAc (50 mL) and the organics were washed with water (20 mL) and brine (20 mL), dried (Na₂SO₄), filtered and concentrated. Flash chromatography (silica, 0-80% EtOAc in iso-hexane) afforded the product (88 mg, 0.118 mmol, 20% yield).

[M+H−^{t}Bu]⁺=525.2/527.2

¹H NMR (DMSO-d6, 400 MHz) δ 1.34 (3H, t, J=7.1 Hz), 1.42 (9H, s), 2.60 (2H, t, J=5.9 Hz), 3.20 (2H, t, J=6.6 Hz), 3.55 (2H, t, J=5.9 Hz), 3.76 (3H, s), 4.05-4.12 (2H, m), 4.36 (2H, q, J=7.1 Hz), 4.46 (3H, s), 6.77 (1H, d, J=1.8 Hz), 6.83 (1H, d, J=1.9 Hz), 7.48-7.65 (3H, m), 7.74 (1H, t, J=1.8 Hz)

### Intermediate 4

### 7-Bromo-2-methyl-isoquinolin-1-one

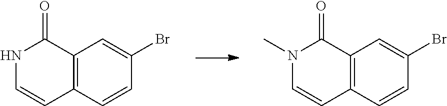

To a solution of 7-bromo-1-hydroxyisoquinoline (500 mg, 2.23 mmol) in THF (20 mL) at rt was added NaH (60% in mineral oil, 107 mg, 2.68 mmol) and Mel (0.42 mL, 6.69 mmol) and the mixture was stirred for 4 h at rt. The solution was diluted with EtOAc (100 mL) and quenched with sat. NH₄Cl (aq). After separation of the layers, the aqueous layer was extracted with EtOAc (2×40 ml) and the combined organic layers were washed with brine (40 mL), dried (Na₂SO₄) and filtered. The solvent was removed to give the product. Flash chromatography (silica, 0-80% EtOAc in heptane) afforded the product (550 mg, 91% yield).

[M+H]⁺=238.0/240.0

¹H NMR (DMSO-d6, 400 MHZ) δ 3.51 (3H, s), 6.64 (1H, dd, J=7.3, 0.7 Hz), 7.53 (1H, d, J=7.3 Hz), 7.64 (1H, d, J=8.5 Hz), 7.85 (1H, dd, J=8.5, 2.2 Hz), 8.28-8.30 (1H, m)

### Intermediate 5

### 6-Bromo-2-methyl-3,4-dihydroisoquinolin-1-one

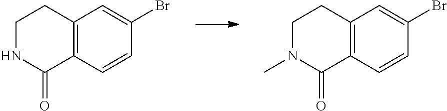

To a solution of 6-bromo-3,4-dihydro-2H-isoquinolin-1-one (500 mg, 2.21 mmol) in dry DMF (11 mL), while cooling in an ice/water bath, was added NaH (60% in mineral oil, 115 mg, 2.88 mmol) and the mixture was stirred at rt for 1 h. Mel (0.18 mL, 2.88 mmol) was added dropwise, and the reaction was stirred for 1 h. Sat. NH₄Cl (aq)(50 mL) was added followed by EtOAc (120 mL). The EtOAc layer was washed with sat. NH₄Cl (aq)(10 mL) and brine (3×20 mL), dried (Na₂SO₄), filtered and concentrated. Flash chromatography (silica, 0-30% EtOAc in iso-hexane) afforded the product (493 mg, 93% yield).

[M+H]⁺=240.0/242.0

¹H NMR (400 MHz, DMSO-d6) δ 2.96-3.00 (2H, m), 3.01 (3H, s), 3.50-3.58 (2H, m), 7.51-7.57 (2H, m), 7.77 (1H, d, J=8.2 Hz)

### Intermediate 6

### 6-Bromo-2-methyl-isoindolin-1-one

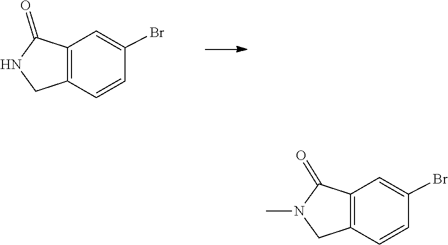

To a solution of 6-bromoisoindolin-1-one (500 mg, 2.36 mmol) in dry DMF (40 mL), while cooling in an ice/water bath, was added NaH (60% in mineral oil, 113 mg, 2.83 mmol) and the mixture was stirred for 15 min. Mel (402 mg, 2.83 mmol) was added dropwise and the reaction was allowed to gradually warm up to rt and left stirring for 18 h. Sat. NH₄Cl (aq)(254 mL) was added followed by EtOAc (150 mL). The EtOAc layer was washed with sat. NH₄Cl (aq)(25 mL) and brine (50 mL), dried (Na₂SO₄), filtered and concentrated. Flash chromatography (silica, 0-80% EtOAc in iso-hexane) afforded the product (288 mg, 54% yield).

[M+H]⁺=226.1/228.1

¹H NMR (400 MHZ, DMSO-d6) δ 3.07 (3H, s), 4.44 (2H, s), 7.55-7.59 (1H, m), 7.72-7.81 (2H, m)

### Intermediate 7

### 7-Bromo-1-methyl-3,4-dihydroquinolin-2-one

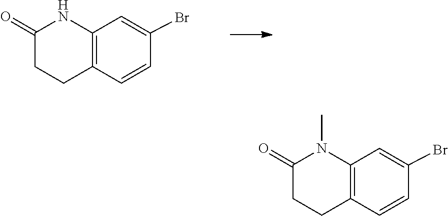

To a solution of 7-bromo-3,4-dihydroquinolin-2 (1H)-one (500 mg, 2.21 mmol) in dry DMF (40 mL) while cooling in an ice/water bath, was added NaH (60% in mineral oil, 106 mg, 2.65 mmol) and the mixture was stirred for 15 min. Mel (377 mg, 2.65 mmol) was added and the mixture was allowed to gradually warm up to rt and left stirring for 18 h. Sat. NH₄Cl (aq)(254 mL) was added followed by EtOAc (150 mL). The EtOAc layer was washed with sat. NH₄Cl (aq)(25 mL) and brine (30 mL), dried (Na₂SO₄), filtered and concentrated to afford the product (580 mg, 100% yield) as an oil.

[M+H]⁺=240.0/242.0

¹H NMR (400 MHZ, DMSO-d6) δ 2.47-2.52 (2H, m), 2.75-2.82 (2H, m), 3.19 (3H, s), 7.11-7.17 (2H, m), 7.20-7.22 (1H, m)

### Intermediate 8

### tert-Butyl 7-bromo-4-[tert-butyl(dimethyl) silyl]oxy-3,4-dihydro-1H-isoquinoline-2-carboxylate

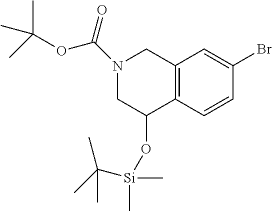 [SMILES: CC(C)(C)OC(=O)N1Cc2cc(Br)ccc2C(O[Si](C)(C)C(C)(C)C)C1]

### 1,4-Dibromo-2-(bromomethyl)benzene

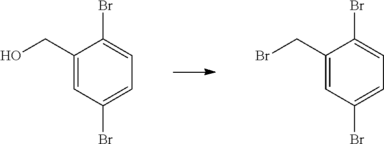 [SMILES: OCc1cc(Br)ccc1Br ; BrCc1cc(Br)ccc1Br]

PBr₃ (0.89 mL, 9.40 mmol) was added to a solution of (2,5-dibromophenyl) methanol (5.00 g, 18.8 mmol) in DCM (125 mL), while cooling in an ice/water bath, and stirred while continuing to cool for 2 h. The mixture was allowed to warm to rt and stirred for a further 20 h. Water (150 ml) was added, and the mixture extracted with DCM (3×25 mL). The DCM fractions were combined and dried (Na₂SO₄). The resulting mixture was filtered and concentrated to give the product (5.01 g, 81% yield) which was used in subsequent reactions without further purification.

¹H NMR (CDCl₃, 400 MHZ) δ 4.53 (2H, s), 7.24-7.33 (1H, m), 7.44 (1H, d, J=8.5 Hz), 7.59 (1H, d, J=2.4 Hz).

### tert-Butyl N-[(2,5-dibromophenyl)methyl]-N-[2-[methoxy(methyl)amino]-2-oxo-ethyl]carbamate

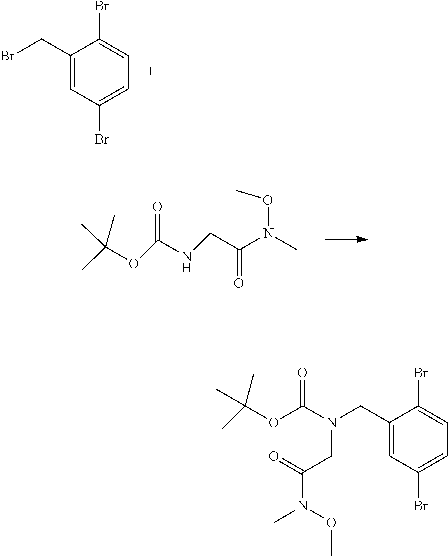 [SMILES: BrCc1cc(Br)ccc1Br ; CON(C)C(=O)CNC(=O)OC(C)(C)C ; CON(C)C(=O)CN(Cc1cc(Br)ccc1Br)C(=O)OC(C)(C)C]

KHMDS (1M in THF, 16.4 mL, 16.4 mmol) was added to DMF (25 mL) at −40° C. The mixture was cooled to −78° C. and a solution of Boc-Gly-N(OMe) Me (3733 mg, 17.1 mmol) in DMF (25 mL) was added slowly. The resulting mixture was then stirred at −78° C. for 15 min. A solution of 1,4-dibromo-2-(bromomethyl)benzene (4500 mg, 13.7 mmol) in THF (25 mL) was slowly added and the resulting mixture was stirred at −78° C. for 1 h. Sat. NH₄Cl (aq)(100 mL) was added and the resulting mixture warmed to rt with stirring. The organics were extracted into EtOAc (2×250 mL), combined, washed with brine (3×300 mL), dried (Na₂SO₄), filtered, and concentrated. Flash chromatography (silica, 0-80% EtOAc in iso-hexane) followed by multiple azeotropes with toluene (5×20 mL) afforded the product (5.74 g, 83% yield).

[M−Boc+H]⁺=365.0/367.0/368.9 with 1:2:1 di-Br splitting pattern.

¹H NMR (CDCl₃, 400 MHZ) δ 1.45 (9H, d, J=18.2 Hz), 3.20 (3H, d, J=1.3 Hz), 3.68 (3H, d, J=17.2 Hz), 4.12 (2H, d, J=57.4 Hz), 4.58 (2H, d, J=29.3 Hz), 7.25-7.30 (1H, m), 7.39 (1H, dd, J=8.4, 5.3 Hz), 7.46 (1H, dd, J=8.4, 2.4 Hz

### tert-Butyl 7-bromo-4-oxo-1,3-dihydroisoquinoline-2-carboxylate

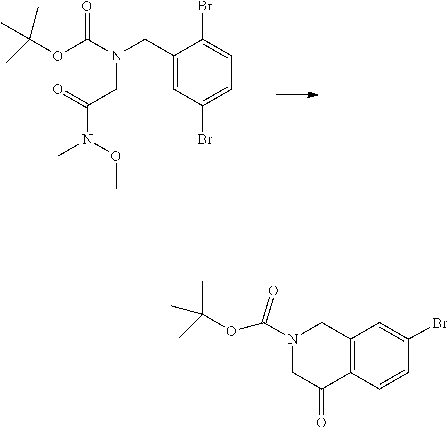 [SMILES: CON(C)C(=O)CN(Cc1cc(Br)ccc1Br)C(=O)OC(C)(C)C ; CC(C)(C)OC(=O)N1CC(=O)c2ccc(Br)cc2C1]

nBuLi (2.5 M in hexanes, 6.8 mL, 17.0 mmol) was added to a stirring solution of tert-butyl N-[(2,5-dibromophenyl)methyl]-N-[2-[methoxy(methyl)amino]-2-oxo-ethyl]carbamate (5.74 g, 11.3 mmol) in THF (100 mL) at −78° C. The mixture was stirred at −78° C. for 1 h. Water (50 mL) was added and the reaction was allowed to warm to rt. The mixture was diluted with water (300 mL) and extracted into EtOAc (3×100 mL). EtOAc fractions were combined, washed with brine (200 ml), dried (Na₂SO₄), filtered and concentrated. Flash chromatography (silica, 0-80% EtOAc in iso-hexane) yielded the product (1856 mg, 49% yield).

¹H NMR (CDCl₃, 400 MHZ) δ 1.47 (9H, s), 4.32 (2H, s), 4.74 (2H, s), 7.46-7.61 (2H, m), 7.91 (1H, d, J=8.4 Hz).

### tert-Butyl 7-bromo-4-hydroxy-3,4-dihydro-1H-isoquinoline-2-carboxylate

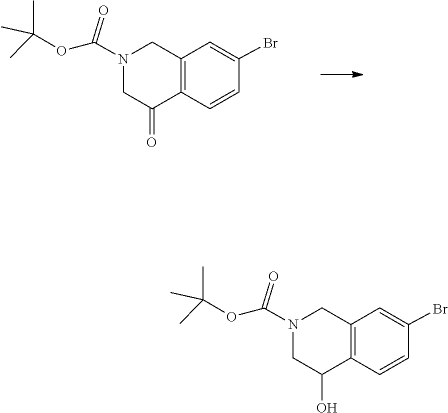 [SMILES: CC(C)(C)OC(=O)N1CC(=O)c2ccc(Br)cc2C1 ; CC(C)(C)OC(=O)N1Cc2cc(Br)ccc2C(O)C1]

NaBH₄ (539 mg, 14.3 mmol) was added to a stirring solution of tert-butyl 7-bromo-4-oxo-1,3-dihydroisoquinoline-2-carboxylate (1200 mg, 3.56 mmol) in MeOH (25 mL) while cooling in an ice/water bath, and stirred for 3 h. Water (100 mL) was added, and the mixture concentrated. Organics were extracted into DCM (3×50 mL). DCM fractions were combined, washed with brine (100 mL), dried (Na₂SO₄), filtered and concentrated to give the product (1100 mg, 92% yield).

¹H NMR (CDCl₃, 400 MHZ) δ 1.49 (9H, s), 3.55-3.65 (1H, m), 3.81-3.93 (1H, m), 4.42 (1H, d, J=17.3 Hz), 4.68-4.77 (2H, m), 7.24-7.36 (2H, m), 7.36-7.47 (1H, m).

### tert-butyl-7-bromo-4-[tert-butyl(dimethyl) silyl]oxy-3,4-dihydro-1H-isoquinoline-2-carboxylate

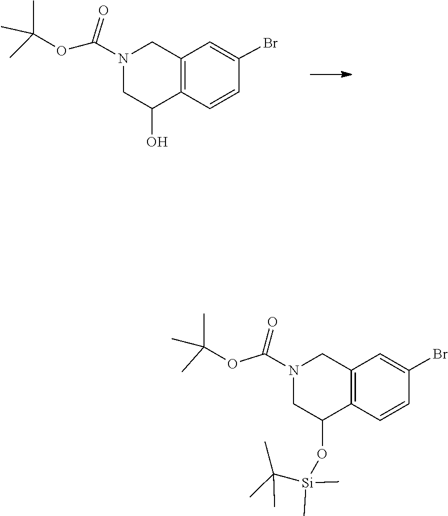 [SMILES: CC(C)(C)OC(=O)N1Cc2cc(Br)ccc2C(O)C1 ; CC(C)(C)OC(=O)N1Cc2cc(Br)ccc2C(O[Si](C)(C)C(C)(C)C)C1]

tert-Butyldimethylsilyl chloride (628 mg, 4.17 mmol) was added to a stirring solution of tert-butyl-7-bromo-4-hydroxy-3,4-dihydro-1H-isoquinoline-2-carboxylate (1100 mg, 3.20 mmol) and imidazole (436 mg, 6.41 mmol) in DCM (30 mL) while cooling in an ice/water bath. The mixture warmed to rt and was stirred for 18 h. Water (50 mL) was added and extracted into DCM (3×25 mL). The DCM fractions were combined, washed with brine (50 mL), dried (Na₂SO₄), filtered and concentrated. Flash chromatography (silica, 0-80% EtOAc in iso-hexane) afforded the product (916 mg, 65% yield).

¹H NMR (CDCl₃, 400 MHz) δ 3 0.15-0.22 (6H, m), 0.94 (9H, s), 1.49 (9H, s), 3.11-3.22 (1H, m), 3.95-4.04 (1H, m), 4.40 (1H, t, J=17.2 Hz), 4.67-4.72 (2H, m), 7.21-7.31 (2H, m), 7.33-7.40 (1H, m).

### Intermediate 9

### Ethyl 6-(2-methyl-1-oxo-1,2,3,4-tetrahydroisoquinolin-7-yl)-7-oxo-4,5,6,7-tetrahydro-1H-pyrazolo[3,4-c]pyridine-3-carboxylate

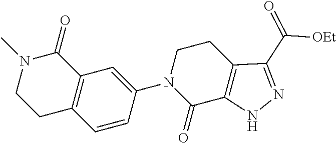 [SMILES: CCOC(=O)c1n[nH]c2c1CCN(c1ccc3c(c1)C(=O)N(C)CC3)C2=O]

### 7-Bromo-2-methyl-3,4-dihydroisoquinolin-1 (2H)-one

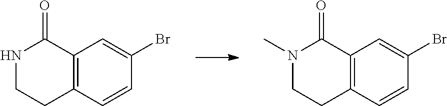

7-Bromo-3,4-dihydro-2H-isoquinolin-1-one (15 g, 66.4 mmol) was dissolved in THF (400 mL) and NaH (60% dispersion in mineral oil, 3.19 g, 79.6 mmol) was added in portions over 5 min. After 10 min stirring, Mel (10.4 g, 73.0 mmol) was added over 5 min and the mixture stirred at rt overnight. The mixture was concentrated and partitioned between EtOAc (100 mL) and sat. NaHCO₃(aq)(100 mL). The organic layer was concentrated and purified by flash chromatography (Silica, 20-65% Pet ether/EtOAc: MeOH (9:1)) to afford the product (13.1 g, 82% yield).

[M+H]⁺=239.9/241.9

¹H NMR (400 MHz, CDCl₃) δ 2.93-2.85 (2H, m), 3.08 (3H, s), 3.49 (2H, dd, J=7.1, 6.3 Hz), 6.98 (1H, dt, J=7.9, 0.9 Hz), 7.44 (1H, dd, J=8.0, 2.2 Hz), 8.14 (1H, d, J=2.2 Hz).

### 2-Methyl-7-(5-morpholino-6-oxo-3,6-dihydropyridin-1 (2H)-yl)-3,4-dihydroisoquinolin-1 (2H)-one

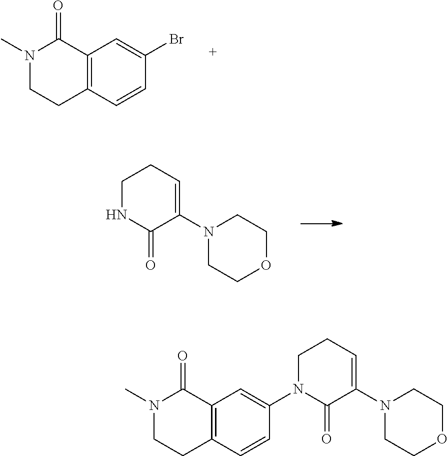 [SMILES: CN1CCc2ccc(Br)cc2C1=O ; O=C1NCCC=C1N1CCOCC1 ; CN1CCc2ccc(N3CCC=C(N4CCOCC4)C3=O)cc2C1=O]

5-Morpholino-2,3-dihydro-1H-pyridin-6-one (6 g, 33 mmol), 7-bromo-2-methyl-3,4-dihydroisoquinolin-1-one (9.5 g, 40 mmol), tripotassium phosphate (10.5 g, 49.4 mmol) and (1S,2S)—N,N′-dimethyl-1,2-cyclohexanediamine (2.81 g, 19.8 mmol) were dissolved in 1,4-dioxane (100 ml). N₂ (g) was bubbled through the mixture for 15 min. Cu(I)I (1.88 g, 9.88 mmol) was added and N₂ (g) bubbled through for 5 min followed by Ar (g) for 3 min. The vessel was sealed and heated to 85° C. for 5 h. After cooling overnight, EtOAc (200 mL) and sat. NH₄Cl (aq)(150 mL) was added. The phases were separated, the aqueous layer extracted with EtOAc (200 mL) and the combined organic layers washed with sat. NH₄Cl (aq)(100 ml). The combined aqueous layers were extracted with additional EtOAc (200 mL) and all organic layers were combined and concentrated. The residue was purified by flash chromatography (Silica, 40-100% Pet ether/EtOAc: MeOH (9:1)) to afford the product (5.5 g, 49%).

[M+H]⁺=342.2

¹H NMR (400 MHZ, CD₃OD) δ 2.54 (2H, td, J=6.8, 4.7 Hz), 2.86-2.82 (4H, m), 3.04 (2H, t, J=6.7 Hz), 3.15 (3H, s), 3.63 (2H, t, J=6.7 Hz), 3.84-3.73 (6H, m), 5.88 (1H, t, J=4.7 Hz), 7.32 (1H, d, J=8.1 Hz), 7.46 (1H, dd, J=8.1, 2.3 Hz), 7.86 (1H, d, J=2.3 Hz)

### Ethyl 2-(2-(4-methoxybenzyl)hydrazineylidene)acetate

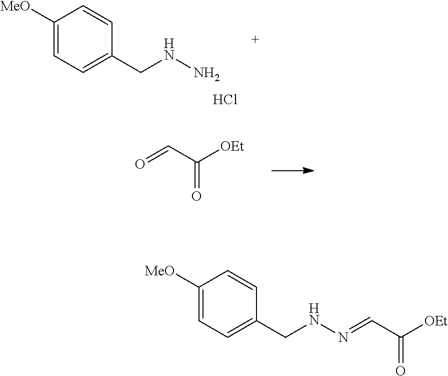 [SMILES: COc1ccc(CNN)cc1 ; Cl ; CCOC(=O)C=O ; CCOC(=O)/C=N/NCc1ccc(OC)cc1]

(4-Methoxyphenyl)methylhydrazine HCl (5 g, 27 mmol) was added to THF (50 mL) followed by TEA (3.22 g, 31.8 mmol) and the mixture stirred at rt for 5 min. Ethyl 2-oxoacetate (5.95 g, 29.2 mmol) was added over 10 min and the resulting mixture stirred at rt overnight. The reaction was filtered and the filtrate evaporated to dryness to afford the product which was used without purification (assuming 100% yield and purity) in the next step.

[M+H]⁺=237.1

### Ethyl 2-chloro-2-(2-(4-methoxybenzyl)hydrazineylidene)acetate

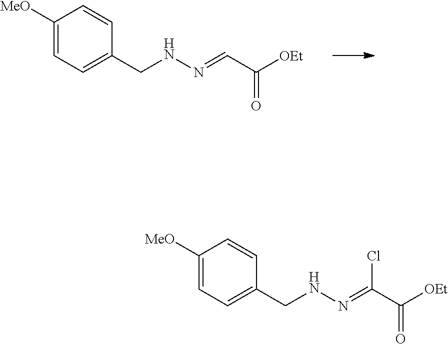 [SMILES: CCOC(=O)/C=N/NCc1ccc(OC)cc1 ; CCOC(=O)/C(Cl)=N/NCc1ccc(OC)cc1]

Ethyl 2-(2-(4-methoxybenzyl) hydrazineylidene)acetate (6.26 g, 26.5 mmol) was dissolved in THF (75 mL). NCS (3.89 g, 29.1 mmol) was added and the reaction stirred at rt overnight. The mixture was evaporated, the residue taken up in Pet Ether/EtOAc (8:1)(30 mL), filtered and purified by flash chromatography (Silica, 10-40% Pet ether/EtOAc) to afford the product (4.2 g, 59% yield).

[M+H+H₂O]⁺=288.1/290.1

¹H NMR (400 MHZ, CDCl₃) δ 1.37 (3H, t, J=7.1 Hz), 3.81 (3H, s), 4.35 (2H, q, J=7.1 Hz), 4.63 (2H, s), 6.62 (1H, s), 6.93-6.86 (2H, m), 7.26-7.22 (2H, m)

### Ethyl 6-(2-methyl-1-oxo-1,2,3,4-tetrahydroisoquinolin-7-yl)-7-oxo-4,5,6,7-tetrahydro-1H-pyrazolo[3,4-c]pyridine-3-carboxylate

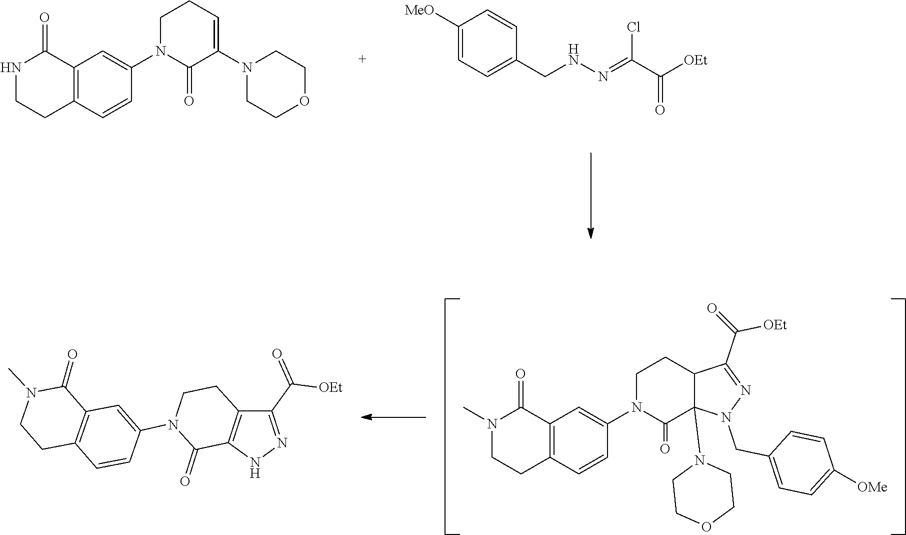

2-Methyl-7-(5-morpholino-6-oxo-3,6-dihydropyridin-1 (2H)-yl)-3,4-dihydroisoquinolin-1 (2H)-one (4.8 g, 14 mmol) was dissolved in 1,4-dioxane (75 mL). K₂CO₃ (4.9 g, 35 mM) was added followed by ethyl 2-chloro-2-(2-(4-methoxybenzyl) hydrazineylidene)acetate (7.23 g, 26.7 mmol) and heated to 85° C. for 18 h. The mixture was cooled, filtered, and washed with EtOAc (50 ml). The combined filtrates were evaporated, dissolved in TFA (27 mL) and heated to 60° C. for 3 h. The mixture was cooled and co-evaporated with DCM (2×100 ml) and toluene (2×100 mL). The residue was taken up in EtOAc (100 ml) and solid K₂CO₃ was added until no more gas evolution occurred. Water (50 ml) and brine (50 mL) were added, and the phases were separated. The aqueous layer was extracted with DCM (2×50 mL) and with 5% MeOH in EtOAc (50 mL). The DCM layer washed with brine (50 mL) and the combined EtOAc layers were filtered and the filtrated washed with brine (50 mL). All organic layers were combined, evaporated and purified by Flash chromatography (Silica, 10-60% DCM/(DCM: 7M NH₃ MeOH 9:1) to afford the product (2.25 g, 43% yield).

[M+H]⁺=369.1

¹H NMR (500 MHZ, CDCl₃) δ 1.42 (3H, t, J=7.1 Hz). 3.02 (2H, t, J=6.7 Hz), 3.17 (3H, s), 3.25 (2H, t, J=6.7 Hz), 3.59 (2H, t, J=6.7 Hz), 4.09 (2H, t, J=6.7 Hz), 4.43 (2H, q, J=7.1 Hz), 7.23 (1H, d, J=8.1 Hz), 7.50 (1H, dd, J=8.0, 2.4 Hz), 7.98 (1H, d, J=2.4 Hz)

### Intermediate 10

### 5-Oxaspiro[2.4]heptan-7-amine hydrochloride

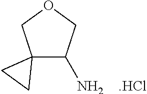

### 4-Aminotetrahydrofuran-3-ol

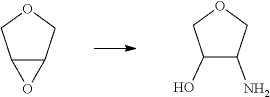 [SMILES: C1OCC2OC12 ; NC1COCC1O]

NH₄OH (28-30% in water, 50 mL, 740 mmol) was added to a solution of 3,6-dioxabicyclo[3.1.0]hexane (3 g, 34.8 mmol) in IPA (10 mL). The mixture was stirred in a sealed tube at 80° C. for 16 h. The reaction mixture was concentrated to afford the product (3.8 g, 95%), which was used in the next step without further purification.

¹H NMR (500 MHZ, DMSO-d6) δ 3.14 (1H, ddd, J=4.7, 2.7, 1.9 Hz), 3.33 (1H, dd, J=8.3, 2.7 Hz), 3.44 (1H, dd, J=8.8, 1.7 Hz), 3.82 (2H, dd, J=5.2, 3.0 Hz), 3.83-3.87 (1H, m). Exchangeable protons are not visible.

### Benzyl N-(4-hydroxytetrahydrofuran-3-yl)carbamate

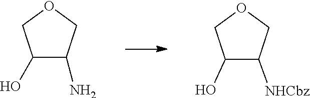 [SMILES: NC1COCC1O ; O=C(NC1COCC1O)OCc1ccccc1]

Benzyl chloroformate (5.21 mL, 36.5 mmol) was added to a suspension of 4-aminotetrahydrofuran-3-ol (3.8 g, 33.2 mmol) and DIPEA (11.3 mL, 66.3 mmol) at 0° C. The reaction mixture was allowed to warm to rt and stirred for 18 h. The mixture was washed sequentially with water (50 mL), sat. NaHCO₃(aq)(50 mL), and brine (50 mL), dried (Na₂SO₄), filtered, and concentrated. The residue was purified by flash chromatography (Silica, 0-100% EtOAc in hexanes) to afford the product (4.2 g, 53% yield).

[M+H]⁺=238.0

¹H NMR (400 MHZ, DMSO-d6) δ 3.41-3.51 (2H m), 3.72-3.81 (2H, m), 3.86 (1H, ddd, J=8.9, 5.5, 1.1 H,), 3.95-4.06 (1H, m), 5.00 (2H, s), 5.17 (1H, dd, J=3.9, 1.3 Hz), 7.24-7.39 (5H, m,), 7.46 (1H, d, J=6.6 Hz).

### Benzyl N-(4-oxotetrahydrofuran-3-yl)carbamate

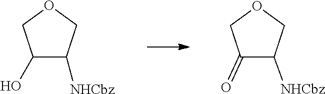 [SMILES: O=C(NC1COCC1O)OCc1ccccc1 ; O=C(NC1COCC1=O)OCc1ccccc1]

Dess-Martin periodinane (15.0 g, 35.4 mmol) was added to a solution of benzyl N-(4-hydroxytetrahydrofuran-3-yl)carbamate (4.20 mg, 17.7 mmol) in DCM (90 mL) at 0° C. The mixture was warmed to rt and stirred for 16 h. This was quenched with sat. NaHCO₃(aq)(100 mL) and extracted with DCM (3×50 mL). The combined organic phases were washed with sat. brine (100 ml), dried (Na₂SO₄), filtered and concentrated. The residue was purified by flash chromatography (Silica, 0-100% EtOAc in hexanes) to afford the product (3.24 g, 74%).

[M+H]⁺=336.0

### Benzyl N-(4-methylenetetrahydrofuran-3-yl)carbamate

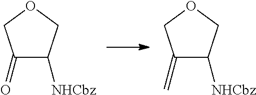 [SMILES: O=C(NC1COCC1=O)OCc1ccccc1 ; C=C1COCC1NC(=O)OCc1ccccc1]

NaHMDS (18.7 mL, 18.7 mmol) was added to a suspension of methyl (triphenyl)phosphonium bromide (6.68 g, 18.7 mmol) in THF (40 mL) at 0° C. The reaction was stirred at rt for 1 h, cooled to 0° C. then benzyl N-(4-oxotetrahydrofuran-3-yl)carbamate (2.0 g, 8.5 mmol) in THF (17 mL) was added dropwise. The reaction mixture was warmed to rt, stirred for 16 h and quenched by sat. NH₄Cl (aq). The mixture was extracted with EtOAc (3×30 mL) and the combined organic layers were dried (Na₂SO₄) and concentrated. Purification by flash chromatography (silica, 0-100% EtOAc in hexanes) afforded the product (70% pure, 530 mg, 19%).

[M+H]⁺=234.0

### Benzyl N-(5-oxaspiro[2.4]heptan-7-yl)carbamate

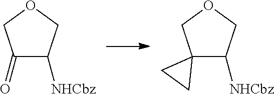 [SMILES: O=C(NC1COCC1=O)OCc1ccccc1 ; O=C(NC1COCC12CC2)OCc1ccccc1]

Diethylzinc (1 M in heptane, 6.22 mL, 6.22 mmol) was added dropwise to a solution of diiodomethane (1.67 g, 6.22 mmol) in DCM (1 mL) at 0° C. After stirring for 15 min, a solution of benzyl N-(4-methylenetetrahydrofuran-3-yl)carbamate (290 mg, 1.24 mmol) in DCM (1 mL) was added dropwise. The reaction was warmed to rt and stirred for 2 h. 1M HCl (aq)(15 mL) was added and the reaction was extracted with DCM (2×15 mL). The combined organic layers were dried (Na₂SO₄), filtered, and concentrated. Purification by flash chromatography (Silica, 0-70% EtOAc in hexanes) afforded the product (190 mg, 62% yield).

[M+H]⁺=248.1

¹H NMR (CDCl₃, 400 MHZ) δ 0.53-0.59 (1H, m), 0.62-0.68 (1H, m), 0.87 (2H, s), 3.53-3.58 (1H, m), 3.80 -3.88 (3H, m), 4.05-4.11 (1H, m), 5.08 (2H, s), 5.10-5.21 (1H, m), 7.35-7.37 (5H, m)

### Oxaspiro[2.4]heptan-7-amine hydrochloride]

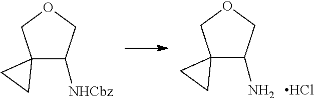 [SMILES: O=C(NC1COCC12CC2)OCc1ccccc1 ; NC1COCC12CC2 ; Cl]

HCl (aq)(1M in EtOAc, 768 μL, 0.768 mmol) was added to a solution of benzyl N-(5-oxaspiro[2.4]heptan-7-yl)carbamate (100 mg, 0.384 mmol) in MeOH (30 mL). The mixture was injected into an H-Cube apparatus and hydrogenated (1.5 mL/min, 20 bar, 20% Pd (OH) 2/C) at 40° C. and subsequently concentrated to afford the product (91 mg, 95% yield).

[M+H]⁺=114.0

¹H NMR (DMSO-d6, 500 MHZ) δ 0.62 (1H, s), 0.82 (1H, s), 1.05-1.42 (2H, m), 3.25-3.50 (2H, m), 3.75-4.24 (3H, m), 8.32 (3H, s)

### Intermediate 11

### 7-Bromo-8-fluoro-4-methyl-2,3-dihydro-1,4-benzoxazepin-5-one

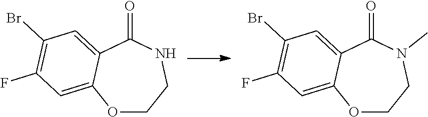

7-Bromo-8-fluoro-3,4-dihydro-2H-1,4-benzoxazepin-5-one (130 mg, 0.5 mmol) in DMF (3 mL) was cooled to 0° C. and NaH (60% oil dispersion, 69 mg, 1.73 mmol) was added and the suspension was stirred at rt for 30 min. After cooling to 0° C., Mel (62 μL, 1.0 mmol) was added and the mixture warmed to rt and stirred for 18 h. After quenching with water (10 mL) and extracting with EtOAc (3×20 mL) the combined organic layers were washed with brine (20 mL), dried (MgSO₄) and concentrated. Purification by flash chromatography (Silica, 0-100% EtOAc in Pet. Ether) afforded the product (78 mg, 57% yield).

[M+H]⁺=273.9/275.9

### Intermediate 12

### 1-(3-lodophenyl)-3-morpholino-5,6-dihydropyridin-2 (1H)-one

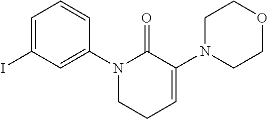 [SMILES: O=C1C(N2CCOCC2)=CCCN1c1cccc(I)c1]

### 5-Bromo-N-(3-iodophenyl)pentanamide

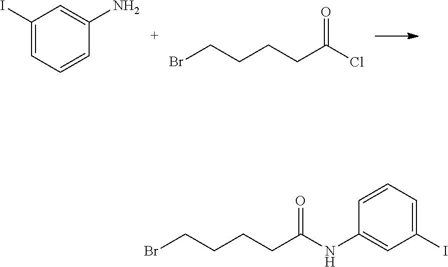 [SMILES: Nc1cccc(I)c1 ; O=C(Cl)CCCCBr ; O=C(CCCCBr)Nc1cccc(I)c1]

To a solution of 3-iodoaniline (25.0 g, 114 mmol) and DIPEA (14.8 g, 114 mmol) in DCM (350 mL), at 0° C.′ was added 5-bromovaleryl chloride (24.9 g, 126 mmol) in DCM (350 mL) over 30 min at 0° C. The mixture was allowed to warm to 5° C.-15° C. and stirred for 4 h. The mixture was washed with 1M HCl (aq)(2×175 mL), water (175 mL), dried (Na₂SO₄), filtered and concentrated to afford the product (42 g) which was taken directly on to the next step.

[M+H]⁺=381.0/383.1

¹H NMR (DMSO-d6, 300 MHZ) δ 1.77-1.88 (4H, m), 2.38 (2H, td, J=5.6, 4.4, 1.7 Hz), 3.66-3.50 (2H, m), 7.18 (1H, t, J=7.9 Hz), 7.30 (1H, ddd, J=8.1, 2.0, 1.1 Hz), 7.60 (1H, dt, J=7.8, 1.4 Hz), 7.67 (1H, t, J=1.8 Hz)

### 1-(3-lodophenyl)piperidin-2-one

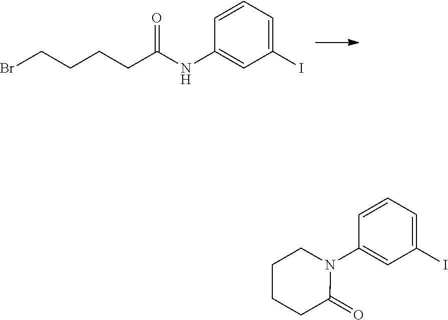 [SMILES: O=C(CCCCBr)Nc1cccc(I)c1 ; O=C1CCCCN1c1cccc(I)c1]

A solution of 5-bromo-N-(3-iodophenyl)pentanamide (42.0 g, 111 mmol) and K₃PO₄ (58.6 g, 279 mmol) in toluene (170 mL) and DMF (42 mL) was heated to 110° C. and stirred for 7 h. The mixture was cooled to rt and brine (420 mL) was added. The organic layer was washed with brine, dried (Na₂SO₄), filtered and concentrated. The residue was purified by slurrying with n-heptane (420 mL) to afford the product (29.0 g, 80% yield over two steps).

[M+H]⁺=301.9

¹H NMR (DMSO-d6, 400 MHZ) δ 1.78-1.88 (4H, m), 2.37 (2H, t, J=6.2 Hz), 3.58 (2H, t, J=5.5 Hz), 7.18 (1H, t, J=7.9 Hz), 7.33-7.27 (1H, m), 7.60 (1H, dt, J=7.9, 1.4 Hz), 7.67 (1H, t, J=1.9 Hz)

### 3,3-Dichloro-1-(3-iodophenyl)piperidin-2-one

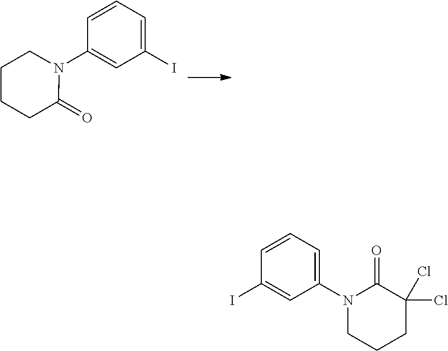 [SMILES: O=C1CCCCN1c1cccc(I)c1 ; O=C1N(c2cccc(I)c2)CCCC1(Cl)Cl]

A solution of 1-(3-iodophenyl)piperidin-2-one (28.0 g, 93.0 mmol) and PCl₅ (96.7 g, 465 mmol) in DCM (1400 mL) was heated to reflux and stirred for 6 h. The mixture was cooled to rt and then poured into ice-cold water. The mixture was extracted with DCM (3×280 mL), dried (Na₂SO₄) and the filtrate was concentrated. The residue was purified by slurrying with MeCN (140 mL) to afford the product (19.5 g, 58% yield).

[M+H]⁺=369.9/371.9

¹H NMR (DMSO-d6, 300 MHZ) δ 2.17-2.04 (2H, m), 2.98-2.86 (2H, m), 3.72 (2H, t, J=6.1 Hz), 7.24 (1H, t, J=7.8 Hz), 7.38-7.30 (1H, m), 7.78-7.65 (2H, m) 1-(3-lodophenyl)-3-morpholino-5,6-dihydropyridin-2 (1H)-one

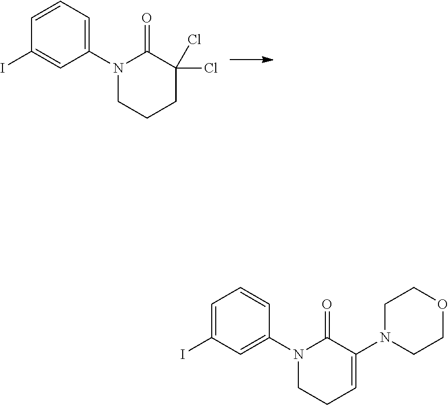 [SMILES: O=C1N(c2cccc(I)c2)CCCC1(Cl)Cl ; O=C1C(N2CCOCC2)=CCCN1c1cccc(I)c1]

A solution of 3,3-dichloro-1-(3-iodophenyl)piperidin-2-one (19.5 g, 53 mmol) and morpholine (19.5 mL) in DMF (39 mL) was heated to 110° C. and stirred for 5-6 h. The reaction was cooled to rt and diluted with toluene (100 mL). The organic phase was washed with water (3×20 mL). The combined organic phases were dried (Na₂SO₄), filtered and concentrated. The residue was purified by slurrying with MeCN (100 mL) and filtered to afford the product (12.6 g, 61% yield).

[M+H]⁺=385.0

¹H NMR (DMSO-d6, 400 MHz) δ 2.43 (2H, td, J=6.6, 4.6 Hz), 2.84-2.72 (4H, m), 3.64 (4H, t, J=4.7 Hz), 3.70 (2H, t, J=6.7 Hz), 5.72 (1H, t, J=4.7 Hz), 7.18 (1H, t, J=8.0 Hz), 7.35 (1H, ddd, J=8.1, 2.1, 1.0 Hz), 7.61-7.52 (1H, m), 7.73 (1H, t, J=1.9 Hz)

### Intermediate 13

### 1-(3-Chlorophenyl)-N-ethyl-6-(3-iodophenyl)-7-oxo-4,5,6,7-tetrahydro-1H-pyrazolo[3,4-c]pyridine-3-carboxamide

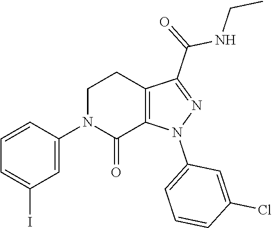 [SMILES: CCNC(=O)c1nn(-c2cccc(Cl)c2)c2c1CCN(c1cccc(I)c1)C2=O]

### Ethyl 1-(3-chlorophenyl)-6-(3-iodophenyl)-7-oxo-4,5,6,7-tetrahydro-1H-pyrazolo[3,4-c]pyridine-3-carboxylate

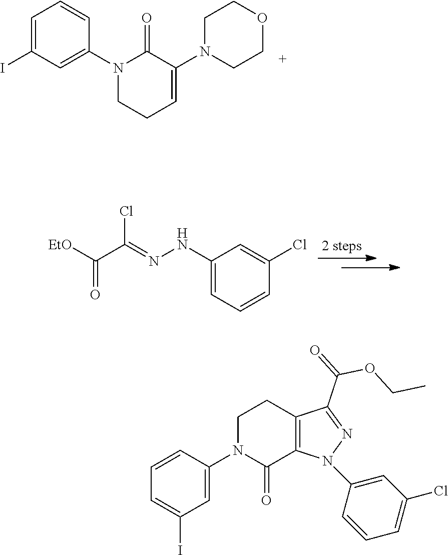 [SMILES: O=C1C(N2CCOCC2)=CCCN1c1cccc(I)c1 ; CCOC(=O)/C(Cl)=N/Nc1cccc(Cl)c1 ; CCOC(=O)c1nn(-c2cccc(Cl)c2)c2c1CCN(c1cccc(I)c1)C2=O]

The product was synthesised following a similar route to ethyl 1-(3-chlorophenyl)-7-oxo-5,6-dihydro-4H-pyrazolo[3,4-c]pyridine-3-carboxylate (Intermediate 1), substituting 3-(morpholin-4-yl)-1,2,5,6-tetrahydropyridin-2-one for 1-(3-iodophenyl)-3-morpholino-5,6-dihydropyridin-2 (1H)-one.

[M+H]⁺=521.9.

¹H NMR (DMSO-d6, 400 MHZ) δ 1.33 (3H, t, J=7.1 Hz), 3.20 (2H, t, J=6.6 Hz), 4.07 (2H, t, J=6.6 Hz), 4.36 (2H, q, J=7.1 Hz), 7.21 (1H, t, J=7.9 Hz), 7.40 (1H, dt, J=8.3, 1.4 Hz), 7.67-7.49 (4H, m), 7.76 (2H, dt, J=4.5, 2.0 Hz)

### 1-(3-Chlorophenyl)-6-(3-iodophenyl)-7-oxo-4,5,6,7-tetrahydro-1H-pyrazolo[3,4-c]pyridine-3-carboxylic acid

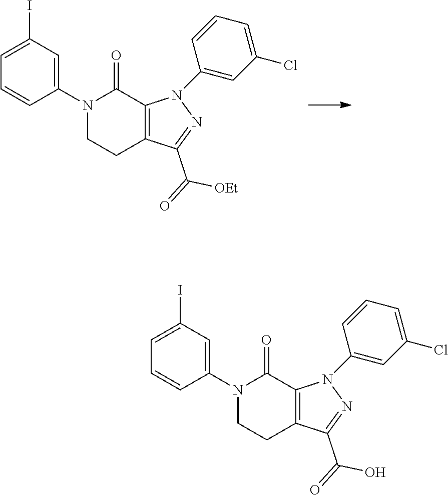 [SMILES: CCOC(=O)c1nn(-c2cccc(Cl)c2)c2c1CCN(c1cccc(I)c1)C2=O ; O=C(O)c1nn(-c2cccc(Cl)c2)c2c1CCN(c1cccc(I)c1)C2=O]

Ethyl 1-(3-chlorophenyl)-6-(3-iodophenyl)-7-oxo-4,5,6,7-tetrahydro-1H-pyrazolo[3,4-c]pyridine-3-carboxylate (500 mg, 0.96 mmol) was dissolved in THF (10 mL), MeOH (1 mL) and water (2 mL) and treated with LiOH·H₂O (192 mg, 3.35 mmol). The mixture was stirred at rt for 2 days. The mixture was concentrated and the residue was diluted with water (20 mL). The mixture was adjusted to pH 2 using 2M HCl (aq), and extracted with EtOAc (3×20 mL). The combined organic layers were washed with brine, dried (MgSO₄) and concentrated to afford the product (473 mg, 100% yield).

[M+H]⁺=493.9

### 1-(3-Chlorophenyl)-N-ethyl-6-(3-iodophenyl)-7-oxo-4,5,6,7-tetrahydro-1H-pyrazolo[3,4-c]pyridine-3-carboxamide

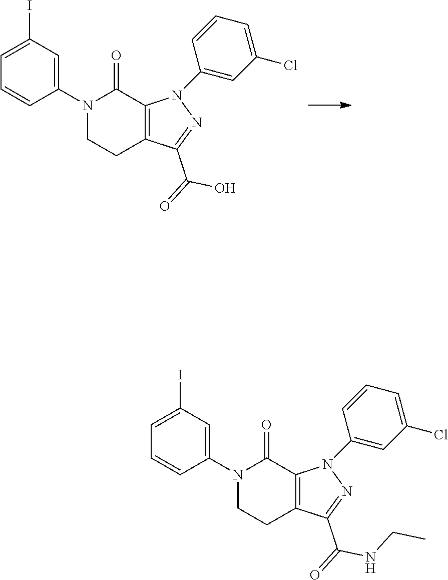 [SMILES: O=C(O)c1nn(-c2cccc(Cl)c2)c2c1CCN(c1cccc(I)c1)C2=O ; CCNC(=O)c1nn(-c2cccc(Cl)c2)c2c1CCN(c1cccc(I)c1)C2=O]

1-(3-Chlorophenyl)-6-(3-iodophenyl)-7-oxo-4,5,6,7-tetrahydro-1H-pyrazolo[3,4-c]pyridine-3-carboxylic acid (460 mg, 0.93 mmol) was dissolved in DCM (15 mL) and DIPEA (487 μL, 2.8 mmol) and treated with HATU (779 mg, 2.05 mmol). The mixture was stirred at rt for 10 min and ethylamine (2M in THF)(932 μL, 1.86 mmol) added. After stirring at rt for 3 days, the mixture was diluted with DCM (30 mL) and sat. NaHCO₃(aq)(30 mL) and the layers were separated. The aqueous layer was re-extracted with DCM (3×15 mL) before the combined organics were washed with sat. NH₄Cl (aq)(10 mL), brine (10 mL), dried (MgSO₄) and concentrated. Purification by flash chromatography (Silica, 0-100% EtOAc in Pet. Ether 60-80) afforded the product (380 mg, 78% yield).

[M+H]⁺=521.0

¹H NMR (DMSO-d6, 400 MHZ) δ 1.11 (3H, t, J=7.2 Hz), 3.20 (2H, t, J=6.6 Hz), 3.25-3.30 (2H, m), 4.05 (2H, t, J=6.6 Hz), 7.21 (1H, t, J=8.0 Hz), 7.37-7.42 (1H, m), 7.50-7.55 (2H, m), 7.60-7.66 (2H, m), 7.74 -7.79 (2H, m), 8.46 (1H, t, J=5.9 Hz)

### Intermediate 14

### 7-Bromo-1,4-dimethyl-3,4-dihydro-1H-benzo[e][1,4]diazepine-2,5-dione

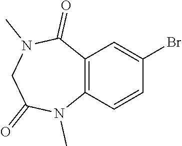 [SMILES: CN1CC(=O)N(C)c2ccc(Br)cc2C1=O]

### 7-Bromo-4-methyl-3,4-dihydro-1H-benzo[e][1,4]diazepine-2,5-dione

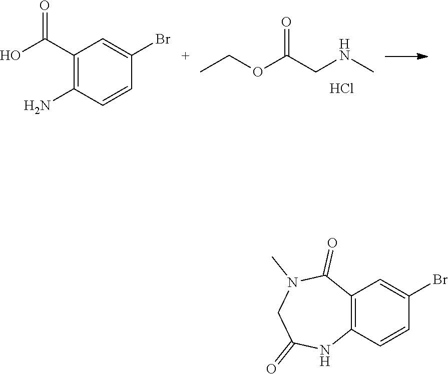

To a solution of 2-amino-5-bromobenzoic acid (1.00 g, 4.63 mmol) in DMF (11 mL) were added sarcosine ethyl ester hydrochloride (1.42 g, 9.26 mmol), DIPEA (4.0 mL, 23.1 mmol) and COMU (1.98 g, 4.63 mmol) and the mixture was stirred at rt for 20 h. The solution was diluted with EtOAc (70 mL) and washed with sat. NaHCO₃(aq)(20 mL) and brine (3×20 mL), dried (Na₂SO₄), filtered and concentrated. Purification by flash column chromatography (Silica, 0-100% of EtOAc in iso-hexanes) afforded the product. (513 mg, 26% yield).

[M−H]⁻=267.1/269.1

¹H NMR (DMSO-d6, 400 MHZ) δ 3.11 (3H, s), 3.88 (2H, s), 7.05 (1H, d, J=8.6 Hz), 7.69 (1H, dd, J=8.7, 2.5 Hz), 7.82 (1H, d, J=2.5 Hz), 10.56 (1H, br s)

### 7-Bromo-1,4-dimethyl-3,4-dihydro-1H-benzo[e][1,4]diazepine-2,5-dione

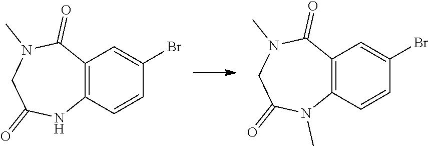 [SMILES: CN1CC(=O)Nc2ccc(Br)cc2C1=O ; CN1CC(=O)N(C)c2ccc(Br)cc2C1=O]

To a solution of 7-bromo-4-methyl-3,4-dihydro-1H-benzo[e][1,4]diazepine-2,5-dione (513 mg, 1.24 mmol) in DMF (6 mL) at 0° C. was added NaH (60% in mineral oil, 74 mg, 1.86 mmol) and the resulting mixture was stirred for 1 h. Mel (0.15 mL, 2.5 mmol) was added and the mixture was stirred for 16 h. After cooling and diluting with EtOAc (70 mL) and water (20 mL), the layers were separated. The organic phase was washed with brine (3×20 mL), dried (Na₂SO₄), filtered, and concentrated. Purification by flash column chromatography (Silica, 0-100% EtOAc in iso-hexane) afforded the product (220 mg, 63% yield).

[M+H]⁺=282.9/284.9

¹H NMR (DMSO-d6, 400 MHZ) δ 3.10 (3H, s), 3.27 (3H, s), 3.72 (1H, d, J=15.1 Hz), 4.12 (1H, d, J=15.0 Hz), 7.34-7.42 (1H, m), 7.75-7.79 (2H, m)

### Intermediate 15

### 7-(3-(Methoxymethyl)-7-oxo-1,4,5,7-tetrahydro-6H-pyrazolo[3,4-c]pyridin-6-yl)-2-methyl-3,4-dihydroisoquinolin-1 (2H)-one

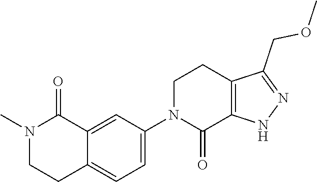

### Ethyl 6-(2-methyl-1-oxo-1,2,3,4-tetrahydroisoquinolin-7-yl)-7-oxo-1-((2-(trimethylsilyl) ethoxy)methyl)-4,5,6,7-tetrahydro-1H-pyrazolo[3,4-c]pyridine-3-carboxylate and

### Ethyl 6-(2-methyl-1-oxo-1,2,3,4-tetrahydroisoquinolin-7-yl)-7-oxo-2-((2-(trimethylsilyl) ethoxy)methyl)-4,5,6,7-tetrahydro-2H-pyrazolo[3,4-c]pyridine-3-carboxylate

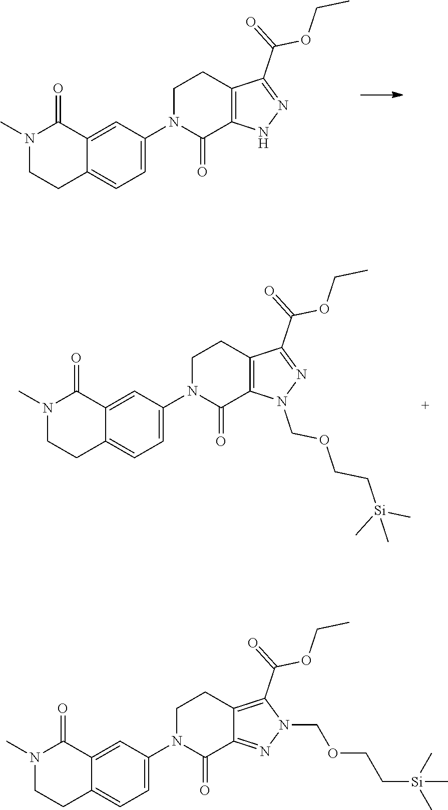

A solution of ethyl 6-(2-methyl-1-oxo-3,4-dihydroisoquinolin-7-yl)-7-oxo-4,5-dihydro-1H-pyrazolo[3,4-c]pyridine-3-carboxylate (5.00 g, 12.9 mmol) in DMF (40 mL) was added to a suspension of NaH (60% in mineral oil, 788 mg, 20.6 mmol) in DMF (10 mL) at 0° C. The mixture was stirred for 15 min at 0° C. and SEM-CI (3 mL, 16.3 mmol) was added. The mixture was warmed to rt, stirred for 18 h and concentrated. The residue was diluted in a mixture of CHCl₃/IPA (4:1 v/v, 100 mL) and washed with brine (3×50 mL). The organic phase was dried (Na₂SO₄), filtered and concentrated. Purification by flash chromatography (Silica, 0-100% EtOAc in hexanes) provided the products (1:1 mixture, 5.13 g, 80% yield).

[M+H]⁺=499.1

### 7-(3-(Hydroxymethyl)-7-oxo-1-((2-(trimethylsilyl) ethoxy)methyl)-1,4,5,7-tetrahydro-6H-pyrazolo[3,4-c]pyridin-6-yl)-2-methyl-3,4-dihydroisoquinolin-1 (2H)-one and

### 7-(3-(Hydroxymethyl)-7-oxo-2-((2-(trimethylsilyl) ethoxy)methyl)-2,4,5,7-tetrahydro-6H-pyrazolo[3,4-c]pyridin-6-yl)-2-methyl-3,4-dihydroisoquinolin-1 (2H)-one

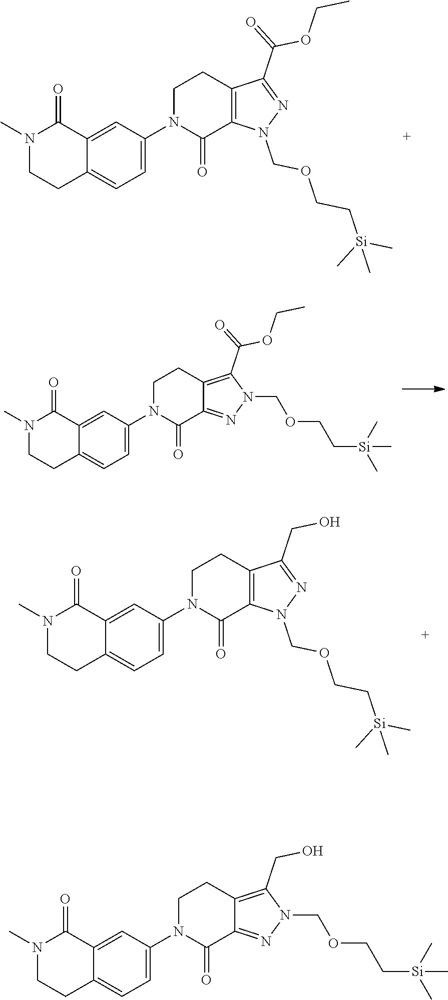

LiBH₄ (2M in THF, 10 mL, 20 mmol) was added to a mixture of ethyl 6-(2-methyl-1-oxo-3,4-dihydroisoquinolin-7-yl)-7-oxo-1-(2-trimethylsilylethoxymethyl)-4,5-dihydropyrazolo[3,4-c]pyridine-3-carboxylate (1:1 mixture with ethyl 6-(2-methyl-1-oxo-3,4-dihydroisoquinolin-7-yl)-7-oxo-2-(2-trimethylsilylethoxymethyl)-4,5-dihydropyrazolo[3,4-c]pyridine-3-carboxylate, 5.10 g, 9.20 mmol) and MeOH (780 μL, 18.9 mmol) in dry THF (30 mL) at 0° C. The mixture was stirred for 15 min at 0° C., warmed to 30° C., stirred for 18 h, quenched with water (3 mL) and concentrated. The residue was diluted in a mixture of CHCl₃/IPA (4:1 v/v, 60 mL) and washed with HCl (aq)(0.5 M, 3×50 mL). The organic phase was dried (Na₂SO₄), filtered and concentrated. Purification by flash chromatography (Silica, 0-100% EtOAc in hexanes, then 0-15% MeOH in EtOAc) provided the products (1:1 mixture, 2.30 g, 55% yield.

[M+H]⁺=457.1

### 7-(3-(Methoxymethyl)-7-oxo-1,4,5,7-tetrahydro-6H-pyrazolo[3,4-c]pyridin-6-yl)-2-methyl-3,4-dihydroisoquinolin-1 (2H)-one

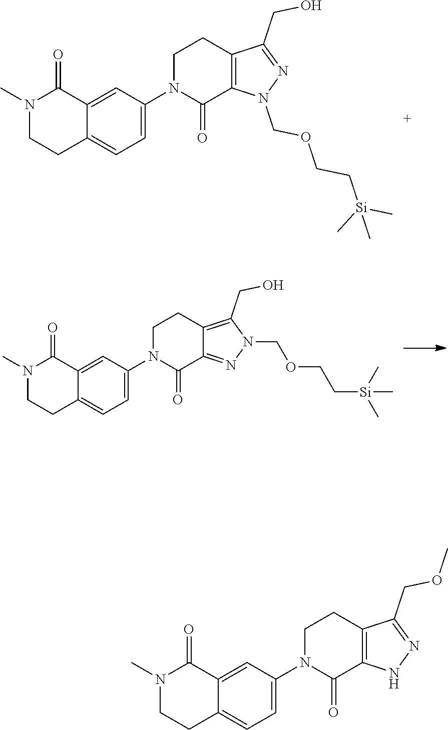

A mixture of 7-[3-(hydroxymethyl)-7-oxo-1-(2-trimethylsilylethoxymethyl)-4,5-dihydropyrazolo[3,4-c]pyridin-6-yl]-2-methyl-3,4-dihydroisoquinolin-1-one (1:1 mixture with 7-[3-(hydroxymethyl)-7-oxo-2-(2-trimethylsilylethoxymethyl)-4,5-dihydropyrazolo[3,4-c]pyridin-6-yl]-2-methyl-3,4-dihydroisoquinolin-1-one, 1.70 g, 3.72 mmol) in THF (15 mL) was added to a mixture of NaH (60% in mineral oil, 428 mg, 11.2 mmol) in THF (5 mL) at 0° C. The mixture was stirred for 15 min and Mel (500 μL, 7.87 mmol) was added dropwise at 0° C. After warming to rt and stirring for 2 h the mixture was quenched with brine (5 mL) and water (5 mL). The layers were separated, and the aqueous layer was extracted with EtOAc (2×10 mL). The combined organic layers were dried (MgSO₄), filtered and concentrated. DCM (20 mL) was added to the residue and TFA (3 mL, 40 mmol) was added dropwise at rt. The mixture was stirred at rt for 4 h and diluted with LiOH (aq)(2M, 20 mL) and a mixture of CHCl₃/IPA (4:1 v/v, 25 mL). The phases were separated and the aqueous phase was extracted with a mixture of CHCl₃/IPA (4:1 v/v, 25 mL). The combined organic phases were concentrated and purified by automated prep HPLC (UV directed, 12-22% MeCN in 10 mM (NH₄) HCO₃ (aq) over 11 min) and lyophilized to provide the product (780 mg, 62% yield). [M+H]⁺=442.2

¹H NMR (DMSO-d6, 500 MHz) δ 2.90 (2H, q, J=6.8 Hz), 2.99 (2H, t, J=6.6 Hz), 3.04 (3H, s), 3.27 (3H, d, J=7.7 Hz), 3.56 (2H, t, J=6.7 Hz), 3.91-4.02 (2H, m), 4.44 (2H, d, J=15.5 Hz), 7.33 (1H, t, J=7.5 Hz), 7.46 (1H, ddd, J=10.9, 8.0, 2.4 Hz), 7.81 (1H, dd, J=11.3, 2.4 Hz), 13.29-13.70 (1H, m)

### Intermediate 16

### 7-(3-(Aminomethyl)-1-(3-chlorophenyl)-7-oxo-1,4,5,7-tetrahydro-6H-pyrazolo[3,4-c]pyridin-6-yl)-2-methyl-3,4-dihydroisoquinolin-1 (2H)-one

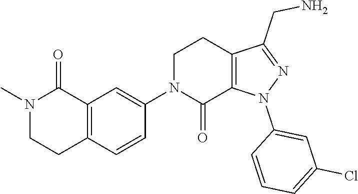 [SMILES: CN1CCc2ccc(N3CCc4c(CN)nn(-c5cccc(Cl)c5)c4C3=O)cc2C1=O]

### 1-(3-Chlorophenyl)-6-(2-methyl-1-oxo-1,2,3,4-tetrahydroisoquinolin-7-yl)-7-oxo-4,5,6,7-tetrahydro-1H-pyrazolo[3,4-c]pyridin-3-yl)methyl methanesulfonate

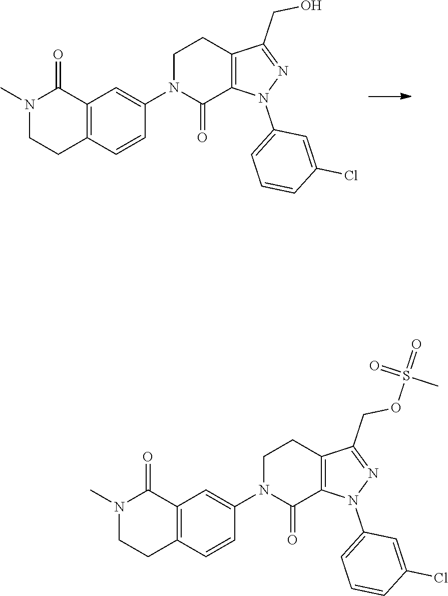 [SMILES: CN1CCc2ccc(N3CCc4c(CO)nn(-c5cccc(Cl)c5)c4C3=O)cc2C1=O ; CN1CCc2ccc(N3CCc4c(COS(C)(=O)=O)nn(-c5cccc(Cl)c5)c4C3=O)cc2C1=O]

Methylsulfonyl methanesulfonate (233 mg, 1.34 mmol) was added to a mixture of 7-[1-(3-chlorophenyl)-3-(hydroxymethyl)-7-oxo-4,5-dihydropyrazolo[3,4-c]pyridin-6-yl]-2-methyl-3,4-dihydroisoquinolin-1-one (390 mg, 0.893 mmol) and TEA (249 μL, 1.79 mmol) in DCM (10 mL) at 0° C. The mixture was allowed to warm to rt and stirred for 15 min. Water (4 mL) was added to the reaction mixture. DCM/MeOH 9:1 (10 mL) was added and the separated organic layer was washed with water (3×20 mL), dried (Na₂SO₄), and concentrated to afford the product (474 mg, 93%).

[M+H]⁺=515.3

¹H NMR (400 MHZ, DMSO-d6) δ 2.98 (2H, t, J=6.7 Hz), 3.03 (3H, s), 3.04-3.08 (2H, m), 3.28 (3H, s), 3.55 (2H, t, J=6.6 Hz), 4.08 (2H, t, J=6.5 Hz), 5.39 (2H, s), 7.29-7.37 (1H, m), 7.43-7.48 (1H, m), 7.48-7.53 (2H, m), 7.55-7.63 (1H, m), 7.69-7.73 (1H, m), 7.82-7.86 (1H, m)

### 7-(3-(Azidomethyl)-1-(3-chlorophenyl)-7-oxo-1,4,5,7-tetrahydro-6H-pyrazolo[3,4-c]pyridin-6-yl)-2-methyl-3,4-dihydroisoquinolin-1 (2H)-one

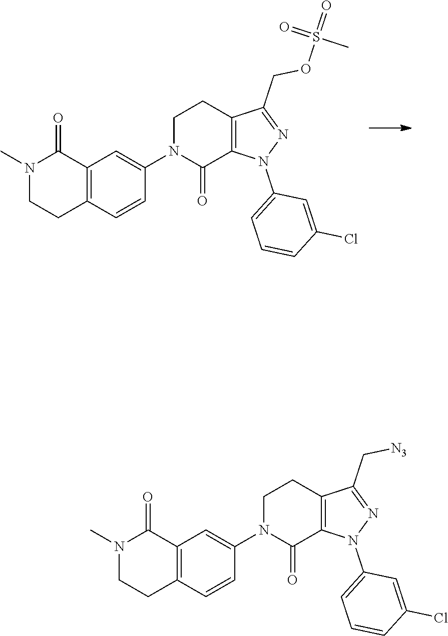 [SMILES: CN1CCc2ccc(N3CCc4c(COS(C)(=O)=O)nn(-c5cccc(Cl)c5)c4C3=O)cc2C1=O ; CN1CCc2ccc(N3CCc4c(CN=[N+]=[N-])nn(-c5cccc(Cl)c5)c4C3=O)cc2C1=O]

Sodium azide (85 mg, 1.31 mmol) was added to a solution of 1-(3-chlorophenyl)-6-(2-methyl-1-oxo-3,4-dihydroisoquinolin-7-yl)-7-oxo-4,5-dihydropyrazolo[3,4-c]pyridin-3-yl]methyl methanesulfonate (225 mg, 0.437 mmol) in DMF (5 mL). The resulting mixture was stirred at 70° C. for 10 min, cooled and EtOAc (15 mL) was added. The mixture was washed with H₂O (3×5 mL), brine (5 ml), dried (Na₂SO₄) and concentrated. Purification by flash chromatography (Silica, 0-10% MeOH in DCM) afforded the product (188 mg, 79%).

[M+H]⁺=461.5

¹H NMR (DMSO-d6, 500 MHZ) δ 2.98 (2H, t, J=6.7 Hz), 3.01-3.05 (5H, m), 3.55 (2H, t, J=6.6 Hz), 4.07 (2H, t, J=6.6 Hz), 4.60 (2H, s), 7.32 (1H, d, J=8.1 Hz), 7.44-7.52 (3H, m), 7.55-7.60 (1H, m), 7.68-7.72 (1H, m), 7.83 (1H, d, J=2.3 Hz)

### 7-(3-(Aminomethyl)-1-(3-chlorophenyl)-7-oxo-1,4,5,7-tetrahydro-6H-pyrazolo[3,4-c]pyridin-6-yl)-2-methyl-3,4-dihydroisoquinolin-1 (2H)-one

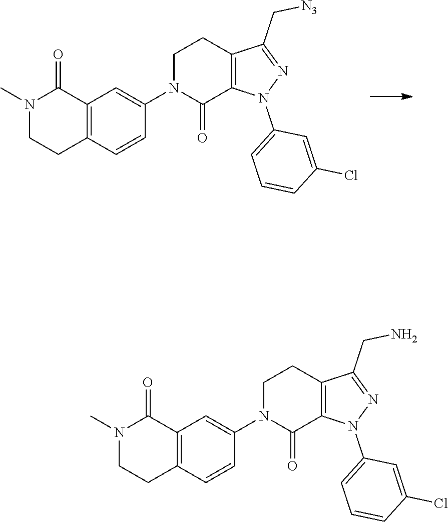 [SMILES: CN1CCc2ccc(N3CCc4c(CN=[N+]=[N-])nn(-c5cccc(Cl)c5)c4C3=O)cc2C1=O ; CN1CCc2ccc(N3CCc4c(CN)nn(-c5cccc(Cl)c5)c4C3=O)cc2C1=O]

Triphenylphosphine bound polymer (3 mmol/g, 204 mg, 0.611 mmol) was added to a mixture of 7-[3-(azidomethyl)-1-(3-chlorophenyl)-7-oxo-4,5-dihydropyrazolo[3,4-c]pyridin-6-yl]-2-methyl-3,4-dihydroisoquinolin-1-one (188 mg, 0.407 mmol) in THF (5 mL) at rt. The mixture was stirred at rt for 6 h and additional triphenylphosphine bound polymer (3 mmol/g, 136 mg, 0.407 mmol) was added. The mixture was stirred at rt for 20 h, additional triphenylphosphine bound polymer (3 mmol/g, 68 mg, 0.204 mmol) was added and stirred at rt for 20 h. Water (73 μL, 4.05 mmol) was added and the mixture was heated at reflux for 3 h, cooled to rt and filtered with Celite®. The resin was washed with a mixture of 10% MeOH in DCM (200 mL) and concentrated to afford the product (167 mg, 75%).

[M+H]⁺=436.1

¹H NMR (DMSO-d6, 500 MHz) δ 2.97 (2H, t, J=6.6 Hz), 3.01-3.08 (5H, m), 3.17 (4H, d, J=4.5 Hz), 3.55 (2H, t, J=6.6 Hz), 4.05 (2H, t, J=6.7 Hz), 7.31 (1H, d, J=8.1 Hz), 7.40-7.49 (3H, m), 7.52-7.56 (1H, m), 7.66 (1H, t, J=2.1 Hz), 7.82 (1H, d, J=2.4 Hz).

### Specific Examples of the Present Invention

### Example number 1001

### N-Benzyl-1-(3-chlorophenyl)-7-oxo-6-(3-(piperidin-1-ylmethyl)phenyl)-4,5,6,7-tetrahydro-1H-pyrazolo[3,4-c]pyridine-3-carboxamide

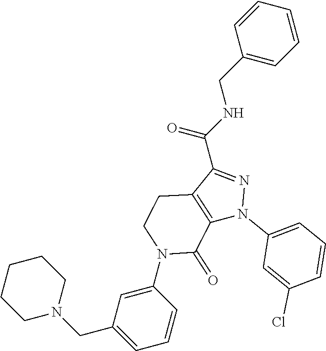 [SMILES: O=C(NCc1ccccc1)c1nn(-c2cccc(Cl)c2)c2c1CCN(c1cccc(CN3CCCCC3)c1)C2=O]

### Ethyl 1-(3-chlorophenyl)-7-oxo-6-[3-(piperidin-1-ylmethyl)phenyl]-4,5-dihydropyrazolo[3,4-c]pyridine-3-carboxylate

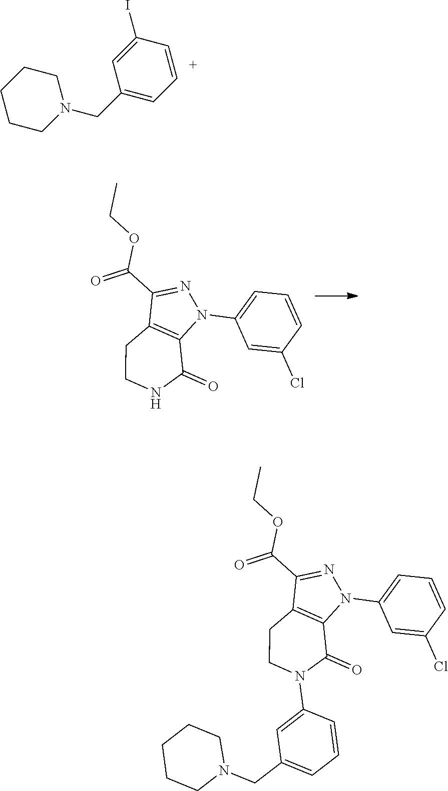 [SMILES: Ic1cccc(CN2CCCCC2)c1 ; CCOC(=O)c1nn(-c2cccc(Cl)c2)c2c1CCNC2=O ; CCOC(=O)c1nn(-c2cccc(Cl)c2)c2c1CCN(c1cccc(CN3CCCCC3)c1)C2=O]

Ethyl 1-(3-chlorophenyl)-4,5,6,7-tetrahydro-7-oxo-1H-pyrazolo[3,4-c]pyridine-3-carboxylate (200 mg, 0.63 mmol) was dissolved in DMSO (3 mL), 1,10-phenanthroline (23 mg, 0.13 mmol), Cu(I)I (131 mg, 0.69 mmol), 1-(3-iodobenzyl)piperidine (207 mg, 0.69 mmol) and K₂CO₃ (173 mg, 1.25 mmol) were added. The mixture was stirred at 130° C. for 4 h then diluted with EtOAc (70 mL). This was washed with water (30 mL), brine (30 mL), dried (Na₂SO₄) and evaporated. The residue was purified by flash chromatography (Silica, 3% MeOH in CHCl₃) to afford the product (178 mg, 58% yield).

[M+H]⁺=493.4

### 1-(3-Chlorophenyl)-7-oxo-6-[3-(piperidin-1-ylmethyl)phenyl]-4,5-dihydropyrazolo[3,4-c]pyridine-3-carboxylic acid

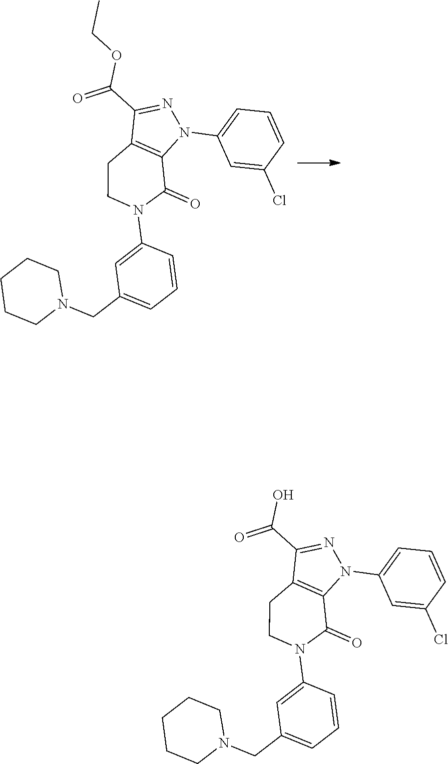 [SMILES: CCOC(=O)c1nn(-c2cccc(Cl)c2)c2c1CCN(c1cccc(CN3CCCCC3)c1)C2=O ; O=C(O)c1nn(-c2cccc(Cl)c2)c2c1CCN(c1cccc(CN3CCCCC3)c1)C2=O]

Ethyl 1-(3-chlorophenyl)-7-oxo-6-[3-(piperidin-1-ylmethyl)phenyl]-4,5-dihydropyrazolo[3,4-c]pyridine-3-carboxylate (178 mg, 0.36 mmol) was dissolved THF (50 mL) and water (5 mL). LiOH·H₂O (47 mg, 1.08 mmol) was added and the mixture was stirred at rt for 18 h. The THF was removed, the pH of the residue was adjusted to pH7 with 2M HCl (aq) and extracted with CHCl₃/IPA (4:1, 3×50 mL). The combined organic extracts were dried (Na₂SO₄) and evaporated to afford the product (165 mg, 98% yield).

[M+H]⁺=465.3

### N-Benzyl-1-(3-chlorophenyl)-7-oxo-6-(3-(piperidin-1-ylmethyl)phenyl)-4,5,6,7-tetrahydro-1H-pyrazolo[3,4-c]pyridine-3-carboxamide

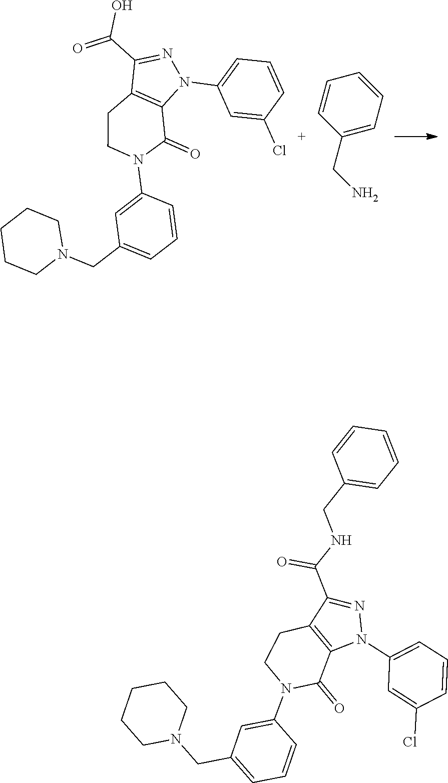

1-(3-Chlorophenyl)-7-oxo-6-[3-(piperidin-1-ylmethyl)phenyl]-4,5-dihydropyrazolo[3,4-c]pyridine-3-carboxylic acid (67 mg, 0.14 mmol) and HBTU (41 mg, 0.17 mmol) were dissolved in DCM (30 mL). TEA (60 μL, 0.43 mmol) was added and after 20 min benzylamine (17 mg, 0.16 mmol) was added. The mixture was stirred at rt for 18 h after which time the mixture was diluted with CHCl₃ (100 mL), washed with sat. NaHCO₃(aq)(30 mL), water (30 mL), brine (30 mL), dried (Na₂SO₄) and evaporated. The residue was purified by flash chromatography (Silica 4% MeOH in CHCl₃), further purified by automated prep HPLC (mass directed 2-60% over 20 min in acidic mobile phase) and lyophilized from MeCN/water to afford the product (65 mg, 75% yield).

[M+H]⁺=554.3

¹H NMR (DMSO-d6, 400 MHz) δ 1.32-1.42 (2H, m), 1.44-1.54 (4H, m), 2.21-2.38 (4H, m), 3.21 (4H, t, J=6.6 Hz), 4.06 (2H, t, J=6.6 Hz), 4.45 (2H, d, J=6.3 Hz), 7.13-7.21 (1H, m), 7.21-7.28 (3H, m), 7.29-7.37 (5H, m), 7.47-7.55 (2H, m), 7.64 (1H, dt, J=6.8, 2.1 Hz), 7.75-7.83 (1H, m), 8.25 (1H, s), 9.02 (1H, t, J=6.3 Hz)

### Example Number 1016

### N-Benzyl-1-(3-chlorophenyl)-6-((1-methylpiperidin-4-yl)methyl)-7-oxo-4,5,6,7-tetrahydro-1H-pyrazolo[3,4-c]pyridine-3-carboxamide

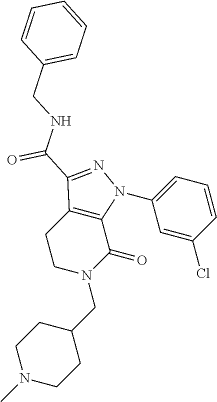 [SMILES: CN1CCC(CN2CCc3c(C(=O)NCc4ccccc4)nn(-c4cccc(Cl)c4)c3C2=O)CC1]

### 1-(3-Chlorophenyl)-6-[[1-[(2-methylpropan-2-yl)oxycarbonyl]piperidin-4-yl]methyl]-7-oxo-4,5-dihydropyrazolo[3,4-c]pyridine-3-carboxylic acid

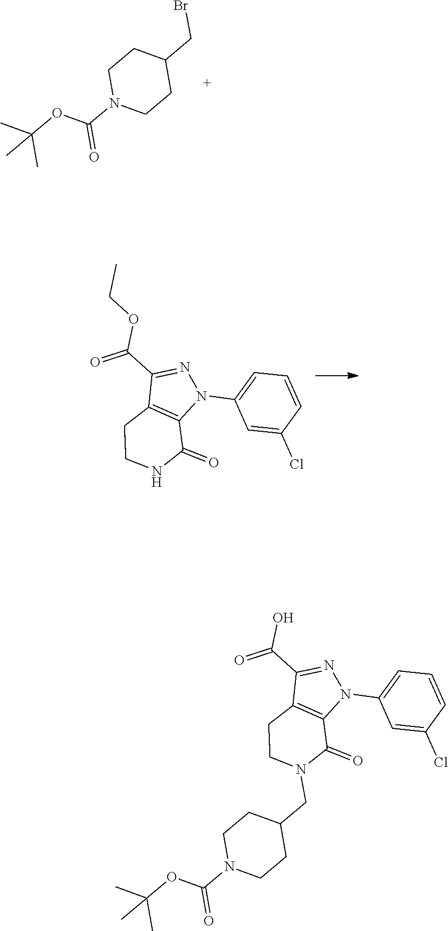 [SMILES: CC(C)(C)OC(=O)N1CCC(CBr)CC1 ; CCOC(=O)c1nn(-c2cccc(Cl)c2)c2c1CCNC2=O ; CC(C)(C)OC(=O)N1CCC(CN2CCc3c(C(=O)O)nn(-c4cccc(Cl)c4)c3C2=O)CC1]

Ethyl 1-(3-chlorophenyl)-4,5,6,7-tetrahydro-7-oxo-1H-pyrazolo[3,4-c]pyridine-3-carboxylate (200 mg, 0.63 mmol) was dissolved in THF (30 mL) at 0° C. NaH (60% oil dispersion)(30 mg, 0.75 mmol) was added and the suspension stirred for 15 min at 0° C. and rt for a further 30 min. The reaction was cooled to 0° C. and N-Boc-4-bromomethyl-piperidine (191 mg, 0.69 mmol) was added and the mixture was allowed to warm to rt over 1 h. The reaction was quenched with 0.3M KHSO₄ (aq)(10 mL) and extracted with EtOAc (2×30 mL). The combined organic extracts were dried (Na₂SO₄) and evaporated. The residue was purified by flash chromatography (Silica, 4% MeOH in CHCl₃) to afford the product (180 mg, 59% yield).

[M+Na]⁺=511.2

### tert-Butyl 4-[[3-(benzylcarbamoyl)-1-(3-chlorophenyl)-7-oxo-4,5-dihydropyrazolo[3,4-c]pyridin-6-yl] methyl]piperidine-1-carboxylate

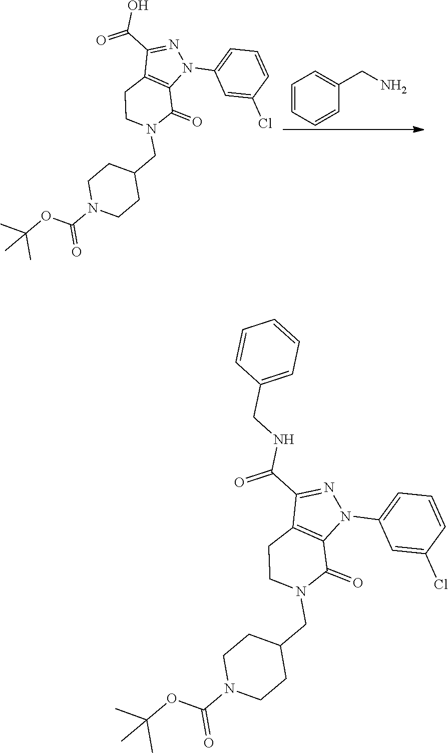

1-(3-Chlorophenyl)-6-[[1-[(2-methylpropan-2-yl)oxycarbonyl]piperidin-4-yl]methyl]-7-oxo-4,5-dihydropyrazolo[3,4-c]pyridine-3-carboxylic acid (180 mg, 0.37 mmol) and HBTU (103 mg, 0.44 mmol) were dissolved in DCM (30 mL). TEA (154 μL, 1.1 mmol) was added. After 20 min benzylamine (43 mg, 0.4 mmol) was added. The mixture was stirred at rt for 18 h after which time the reaction mixture was diluted with CHCl₃ (100 mL). This solution was washed with sat. NaHCO₃ (30 mL), water (30 mL), brine (30 mL), dried (Na₂SO₄) and evaporated. The residue was purified by flash chromatography (Silica, 2% MeOH in CHCl₃) to afford the product (132 mg, 62% yield).

[M−Boc+H]⁺=478.5

### N-Benzyl-1-(3-chlorophenyl)-6-((1-methylpiperidin-4-yl)methyl)-7-oxo-4,5,6,7-tetrahydro-1H-pyrazolo[3,4-c]pyridine-3-carboxamide formate

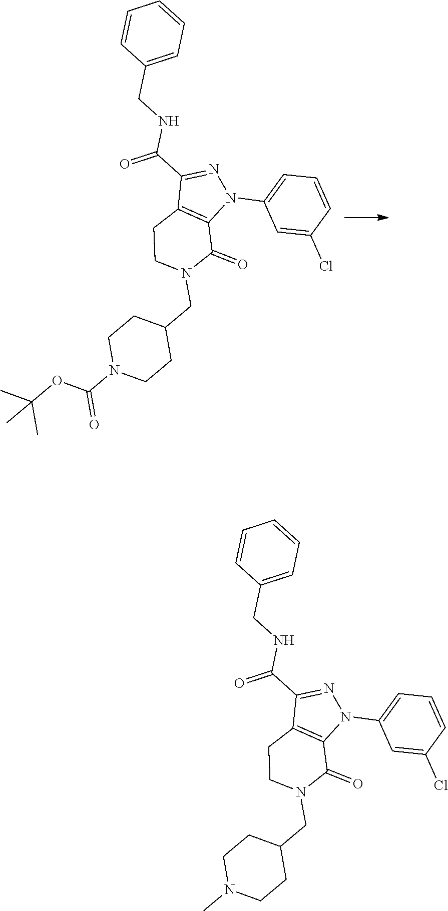 [SMILES: CC(C)(C)OC(=O)N1CCC(CN2CCc3c(C(=O)NCc4ccccc4)nn(-c4cccc(Cl)c4)c3C2=O)CC1 ; CN1CCC(CN2CCc3c(C(=O)NCc4ccccc4)nn(-c4cccc(Cl)c4)c3C2=O)CC1]

A solution of tert-butyl 4-[[3-(benzylcarbamoyl)-1-(3-chlorophenyl)-7-oxo-4,5-dihydropyrazolo[3,4-c]pyridin-6-yl]methyl]piperidine-1-carboxylate (80 mg, 0.14 mmol) in formic acid (52 μL, 1.38 mmol) was stirred at 50° C. for 30 min, formaldehyde (37% in water)(13 μL, 0.35 mmol) was added and the mixture heated to 90° C. for 2 h. The reaction mixture was allowed to cool to rt and evaporated. The residue was purified by automated prep HPLC (mass directed 2-60% MeCN in water with an acidic mobile phase of 2.5% formic acid in water to afford the product (22 mg, 30% yield).

[M+H]⁺=492.2

¹H NMR (DMSO-d6, 400 MHZ) δ 0.93 (1H, t, J=7.1 Hz), 1.09-1.21 (2H, m), 1.54-1.60 (2H, m), 1.79 (2H, td, J=11.7, 2.3 Hz), 2.11 (3H, s), 2.71 (2H, dt, J=11.6, 3.3 Hz), 3.04 (2H, t, J=6.7 Hz), 3.27 (2H, d, J=6.6 Hz), 3.65 (2H, d, J=6.7 Hz), 4.42 (2H, d, J=6.3 Hz), 7.22 (1H, ddd, J=8.8, 5.0, 3.8 Hz), 7.31 (3H, d, J=4.3 Hz), 7.49-7.54 (2H, m), 7.58 (1H, dt, J=6.3, 2.3 Hz), 7.71-7.74 (1H, m), 8.42 (3H, s), 8.96 (1H, t, J=6.3 Hz)

### Example number 1024

### (S)-1-(3-Chlorophenyl)-N-(2-hydroxy-1-phenylethyl)-6-(2-methyl-1,2,3,4-tetrahydroisoquinolin-7-yl)-7

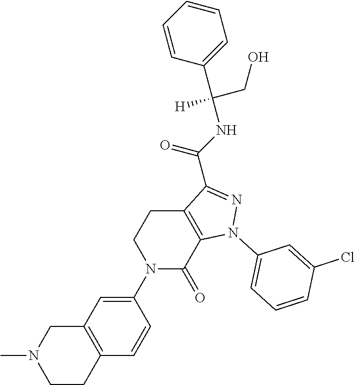 [SMILES: CN1CCc2ccc(N3CCc4c(C(=O)N[C@H](CO)c5ccccc5)nn(-c5cccc(Cl)c5)c4C3=O)cc2C1]

### Ethyl 1-(3-chlorophenyl)-6-(2-methyl-3,4-dihydro-1H-isoquinolin-7-yl)-7-oxo-4,5-dihydropyrazolo[3,4-c]pyridine-3-carboxylate

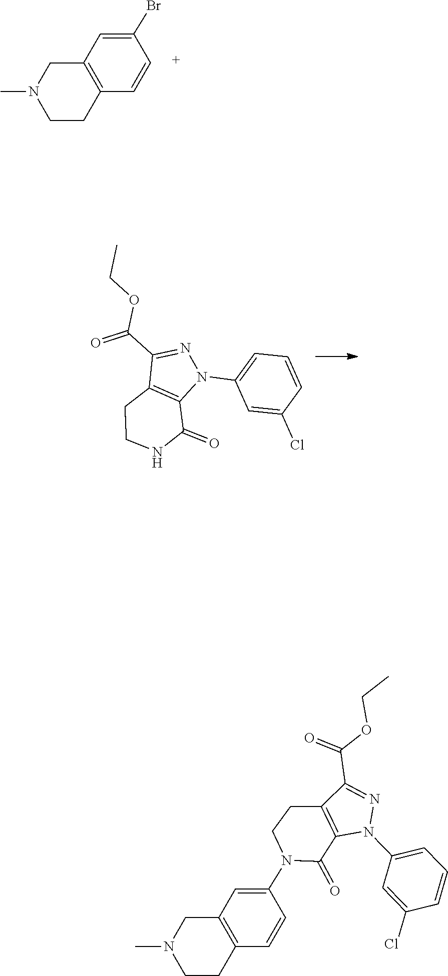 [SMILES: CN1CCc2ccc(Br)cc2C1 ; CCOC(=O)c1nn(-c2cccc(Cl)c2)c2c1CCNC2=O ; CCOC(=O)c1nn(-c2cccc(Cl)c2)c2c1CCN(c1ccc3c(c1)CN(C)CC3)C2=O]

Ethyl 1-(3-chlorophenyl)-4,5,6,7-tetrahydro-7-oxo-1H-pyrazolo[3,4-c]pyridine-3-carboxylate (2500 mg, 7.82 mmol) was dissolved in DMSO (3 mL) and 1,10-phenanthroline (282 mg, 1.56 mmol), Cu(I)I (1638 mg, 8.6 mmol), 7-bromo-2-methyl-3,4-dihydro-1H-isoquinoline (1945 mg, 8.6 mmol) and K₂CO₃ (2161 mg, 15.64 mmol) were added. The mixture was stirred at 130° C. for 30 min and then diluted with EtOAc (70 mL). This solution was washed with water, brine, dried (Na₂SO₄) and evaporated. The residue was purified by flash chromatography (Silica, 3% MeOH in CHCl₃) to afford the product (2620 mg, 72% yield).

[M+H]⁺=465.2

### 1-(3-Chlorophenyl)-6-(2-methyl-3,4-dihydro-1H-isoquinolin-7-yl)-7-oxo-4,5-dihydropyrazolo[3,4-c] pyridine-3-carboxylic acid

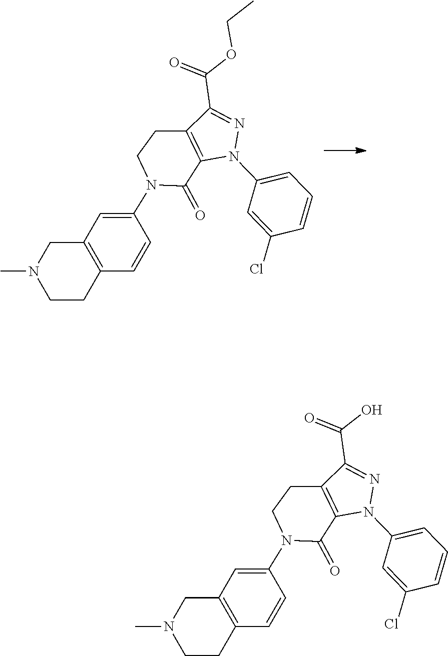 [SMILES: CCOC(=O)c1nn(-c2cccc(Cl)c2)c2c1CCN(c1ccc3c(c1)CN(C)CC3)C2=O ; CN1CCc2ccc(N3CCc4c(C(=O)O)nn(-c5cccc(Cl)c5)c4C3=O)cc2C1]

Ethyl 1-(3-chlorophenyl)-6-(2-methyl-3,4-dihydro-1H-isoquinolin-7-yl)-7-oxo-4,5-dihydropyrazolo[3,4-c] pyridine-3-carboxylate (2600 mg, 5.59 mmol) was dissolved THF (100 mL) and water (10 mL). LiOH·H₂O (721 mg, 16.8 mmol) was added and the mixture stirred at rt for 3 h. The THF was removed, the pH of the residue was adjusted to pH7 with 2M HCl (aq) and extracted with CHCl₃/IPA (4:1, 3×50 mL). The combined organic extracts were dried (Na₂SO₄) and evaporated to afford the product (2320 mg, 95% yield).

[M+H]⁺=437.1

### (S)-1-(3-Chlorophenyl)-N-(2-hydroxy-1-phenylethyl)-6-(2-methyl-1,2,3,4-tetrahydroisoquinolin-7-yl)-7-oxo-4,5,6,7-tetrahydro-1H-pyrazolo[3,4-c]pyridine-3-carboxamide

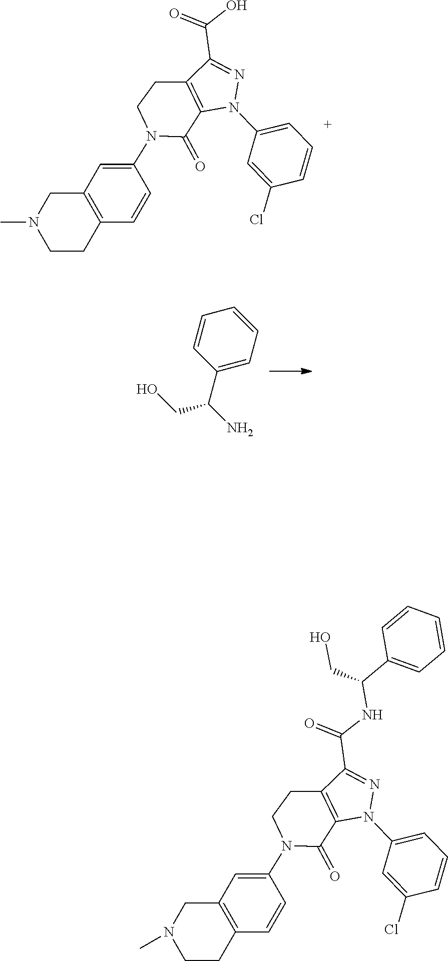 [SMILES: CN1CCc2ccc(N3CCc4c(C(=O)O)nn(-c5cccc(Cl)c5)c4C3=O)cc2C1 ; N[C@H](CO)c1ccccc1 ; CN1CCc2ccc(N3CCc4c(C(=O)N[C@H](CO)c5ccccc5)nn(-c5cccc(Cl)c5)c4C3=O)cc2C1]

1-(3-Chlorophenyl)-6-(2-methyl-3,4-dihydro-1H-isoquinolin-7-yl)-7-oxo-4,5-dihydropyrazolo[3,4-c] pyridine-3-carboxylic acid (63 mg, 0.14 mmol) and HBTU (41 mg, 0.17 mmol) were dissolved in DCM (30 mL). TEA (60 μL, 0.43 mmol), was added. After 20 min(S)-2-phenylglycinol (22 mg, 0.16 mmol) was added. The reaction mixture was stirred at rt for 18 h then diluted with CHCl₃ (100 mL). This solution was washed with sat. NaHCO₃ (30 mL), water (30 mL), brine (30 mL), dried (Na₂SO₄) and evaporated. The residue was purified by flash chromatography (Silica, 4% MeOH in CHCl₃) and lyophilized from MeCN/water to afford the product (38 mg, 47% yield).

[M+H]⁺=556.2

¹H NMR (DMSO-d6, 400 MHZ) δ 2.32 (3H, s), 2.57 (2H, t, J=5.9 Hz), 2.79 (2H, t, J=5.9 Hz), 3.11-3.17 (2H, m), 3.44 (2H, s), 3.64-3.81 (2H, m), 4.00 (2H, t, J=6.6 Hz), 5.00 (1H, t, J=5.6 Hz), 5.06 (1H, td, J=7.7, 5.2 Hz), 6.98-7.04 (1H, m), 7.07-7.13 (2H, m), 7.19-7.27 (1H, m), 7.29-7.35 (2H, m), 7.37-7.42 (2H, m), 7.49-7.56 (2H, m), 7.64 (1H, dt, J=6.9, 2.0 Hz), 7.78-7.83 (1H, m), 8.61 (1H, d, J=8.3 Hz)

### Example Number 1034

### 1-(3-Chlorophenyl)-N-(cyclopropylmethyl)-6-(7-methyl-5,6,7,8-tetrahydro-1,7-naphthyridin-2-yl)-7-oxo-4,5,6,7-tetrahydro-1H-pyrazolo[3,4-c]pyridine-3-carboxamide

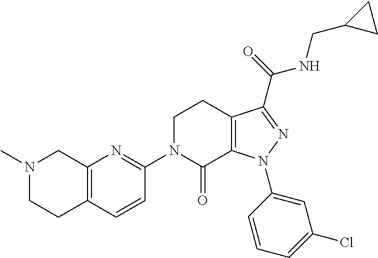 [SMILES: CN1CCc2ccc(N3CCc4c(C(=O)NCC5CC5)nn(-c5cccc(Cl)c5)c4C3=O)nc2C1]

### tert-Butyl 2-[1-(3-chlorophenyl)-3-ethoxycarbonyl-7-oxo-4,5-dihydropyrazolo[3,4-c]pyridin-6-yl]-6,8-dihydro-5H-1,7-naphthyridine-7-carboxylate

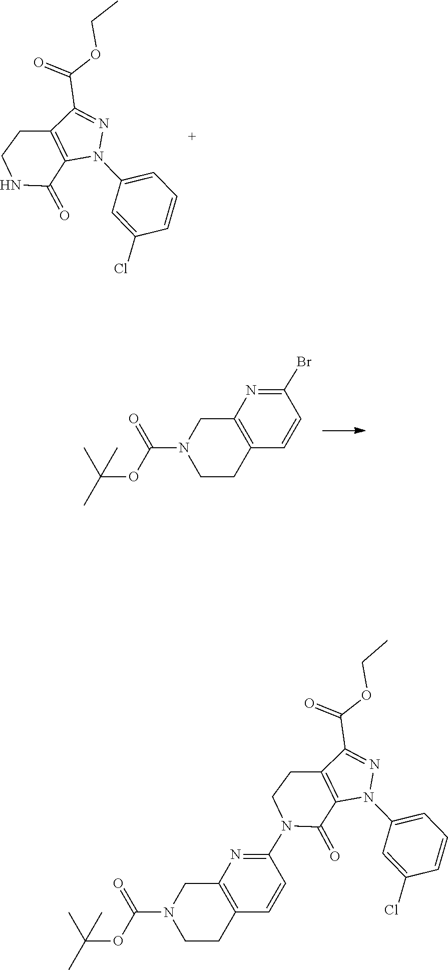 [SMILES: CCOC(=O)c1nn(-c2cccc(Cl)c2)c2c1CCNC2=O ; CC(C)(C)OC(=O)N1CCc2ccc(Br)nc2C1 ; CCOC(=O)c1nn(-c2cccc(Cl)c2)c2c1CCN(c1ccc3c(n1)CN(C(=O)OC(C)(C)C)CC3)C2=O]

A solution of ethyl 1-(3-chlorophenyl)-7-oxo-5,6-dihydro-4H-pyrazolo[3,4-c]pyridine-3-carboxylate (125 mg, 0.39 mmol) and tert-butyl 2-chloro-6,8-dihydro-5H-1,7-naphthyridine-7-carboxylate (126 mg, 0.47 mmol) in dry 1,4-dioxane (4 mL) was purged with N₂ (g) for 5 min. K₃PO₄ (207 mg, 0.98 mmol) and (15,2S)—N, N′-dimethyl-1,2-cyclohexanediamine (22 mg, 0.16 mmol) were sequentially added followed by Cu(I)I (15 mg, 0.078 mmol). The sealed vessel was stirred at 100° C. for 18 h. The mixture was filtered through

Celite®, the residue washed with EtOAc (50 mL); the filtrate was washed with water and brine, dried (Na₂SO₄) and evaporated. The residue was purified by flash chromatography (silica, 0-60% EtOAc in iso-hexane) to afford the product (70 mg, 32% yield).

[M+H]⁺=552.0, 554.0

¹H NMR (DMSO-d6, 400 MHZ) δ 1.33 (3H, t, J=7.1 Hz), 1.44 (9H, s), 2.77 (2H, t, J=5.8 Hz), 3.16 (2H, t, J=6.5 Hz), 3.60 (2H, t, J=5.7 Hz), 4.31 (2H, t, J=6.6 Hz), 4.36 (2H, q, J=7.1 Hz), 4.50 (2H, s), 7.48-7.65 (5H, m), 7.79 (1H, t, J=1.8 Hz)

### 6-(7-tert-Butoxycarbonyl-6,8-dihydro-5H-1,7-naphthyridin-2-yl)-1-(3-chlorophenyl)-7-oxo-4,5-dihydropyrazolo[3,4-c]pyridine-3-carboxylic acid

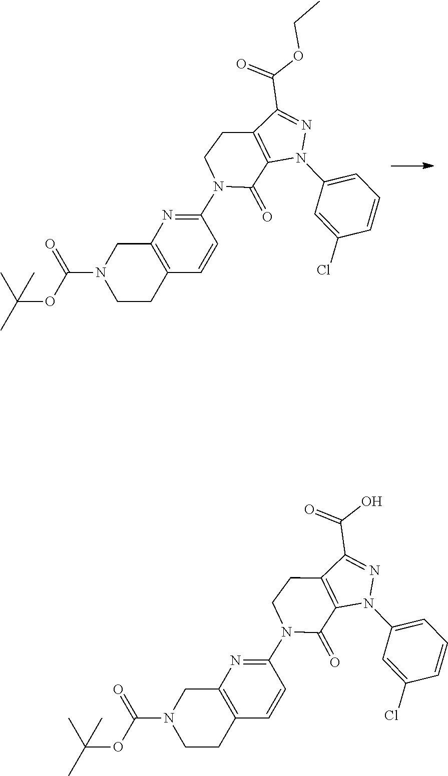 [SMILES: CCOC(=O)c1nn(-c2cccc(Cl)c2)c2c1CCN(c1ccc3c(n1)CN(C(=O)OC(C)(C)C)CC3)C2=O ; CC(C)(C)OC(=O)N1CCc2ccc(N3CCc4c(C(=O)O)nn(-c5cccc(Cl)c5)c4C3=O)nc2C1]

To a solution of tert-butyl 2-[1-(3-chlorophenyl)-3-ethoxycarbonyl-7-oxo-4,5-dihydropyrazolo[3,4-c]pyridin-6-yl]-6,8-dihydro-5H-1,7-naphthyridine-7-carboxylate (70 mg, 0.13 mmol) in a mixture of THF (3 mL) and water (1 mL) was added LiOH·H₂O (13 mg, 0.32 mmol). The mixture was stirred at rt for 72 h. The THF was removed, and the remaining aqueous solution was acidified to pH=2-3 using 1M HCl (aq). This was extracted with EtOAc (3×40 mL) and the combined organic extracts were dried (Na₂SO₄) and evaporated to afford the product (57 mg, 85% yield).

[M+H]⁺=524.1, 526.1

### tert-Butyl 7-[1-(3-chlorophenyl)-3-(cyclopropylmethylcarbamoyl)-7-oxo-4,5-dihydropyrazolo[3,4-c]pyridin-6-yl]-3,4-dihydro-1H-isoquinoline-2-carboxylate

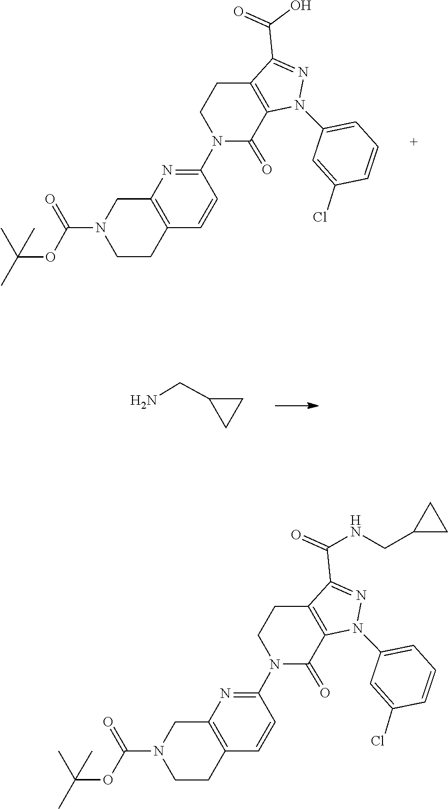 [SMILES: CC(C)(C)OC(=O)N1CCc2ccc(N3CCc4c(C(=O)O)nn(-c5cccc(Cl)c5)c4C3=O)nc2C1 ; NCC1CC1 ; CC(C)(C)OC(=O)N1CCc2ccc(N3CCc4c(C(=O)NCC5CC5)nn(-c5cccc(Cl)c5)c4C3=O)nc2C1]

To a suspension of 6-(7-tert-butoxycarbonyl-6,8-dihydro-5H-1,7-naphthyridin-2-yl)-1-(3-chlorophenyl)-7-oxo-4,5-dihydropyrazolo[3,4-c]pyridine-3-carboxylic acid (55 mg, 0.1 mmol) in MeCN (12 mL) were added aminomethylcyclopropane (13 μL, 0.15 mmol), 1-methylimidazole (29 μL, 0.32 mmol) and TCFH (38 mg, 0.14 mmol). The mixture was stirred at rt for 20 h. This solution was diluted with EtOAc (75 mL), washed with sat. NaHCO₃(aq) and brine, dried (Na₂SO₄) and evaporated. The residue was purified by flash chromatography (silica, 0-40% EtOAc in iso-hexane) to afford the product (56 mg, 92% yield).

[M+H]⁺=577.2, 579.2

¹H NMR (DMSO-d6, 400 MHZ) δ 0.21-0.27 (2H, m), 0.38-0.47 (2H, m), 0.99-1.11 (1H, m), 1.44 (9H, s), 2.77 (2H, t, J=5.8 Hz), 3.12 (2H, t, J=6.5 Hz), 3.17 (2H, t, J=6.5 Hz), 3.60 (2H, t, J=5.8 Hz), 4.29 (2H, t, J=6.5 Hz), 4.50 (2H, s), 7.48-7.61 (4H, m), 7.65 (1H, dt, J=6.8, 2.1 Hz), 7.80-7.84 (1H, m), 8.52 (1H, t, J=6.0 Hz)

### 1-(3-Chlorophenyl)-N-(cyclopropylmethyl)-7-oxo-6-(5,6,7,8-tetrahydro-1,7-naphthyridin-2-yl)-4,5-dihydropyrazolo[3,4-c]pyridine-3-carboxamide hydrochloride

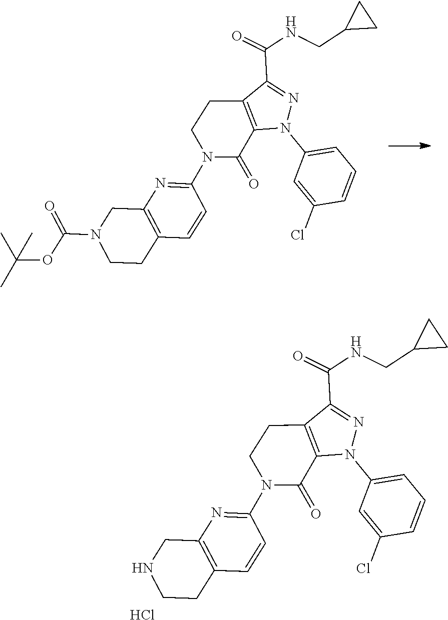 [SMILES: CC(C)(C)OC(=O)N1CCc2ccc(N3CCc4c(C(=O)NCC5CC5)nn(-c5cccc(Cl)c5)c4C3=O)nc2C1 ; O=C(NCC1CC1)c1nn(-c2cccc(Cl)c2)c2c1CCN(c1ccc3c(n1)CNCC3)C2=O ; Cl]

tert-Butyl 7-[1-(3-chlorophenyl)-3-(cyclopropylmethylcarbamoyl)-7-oxo-4,5-dihydropyrazolo[3,4-c]pyridin-6-yl]-3,4-dihydro-1H-isoquinoline-2-carboxylate (50 mg, 0.09 mmol) was dissolved in 4M HCl in 1,4-dioxane (3 mL). After 20 h at rt the solvent was removed to afford the product (42 mg, 95% yield).

[M+H]⁺=477.2, 479.2

¹H NMR DMSO-d6, 400 MHZ) δ 0.19-0.28 (2H, m), 0.38-0.46 (2H, m), 0.99-1.10 (1H, m), 3.01 (2H, t, J=6.1 Hz), 3.12 (2H, t, J=6.4 Hz), 3.18 (2H, t, J=6.5 Hz), 3.39-3.44 (2H, m), 4.23-4.35 (4H, m), 7.50-7.57 (2H, m), 7.60-7.71 (3H, m), 7.80-7.86 (1H, m), 8.54 (1H, t, J=6.0 Hz), 9.49 (2H, s br)

### 1-(3-Chlorophenyl)-N-(cyclopropylmethyl)-6-(7-methyl-5,6,7,8-tetrahydro-1,7-naphthyridin-2-yl)-7-oxo-4,5,6,7-tetrahydro-1H-pyrazolo[3,4-c]pyridine-3-carboxamide

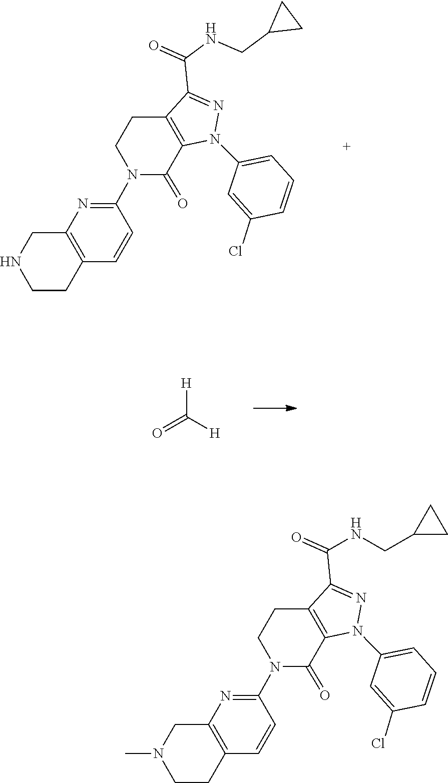 [SMILES: O=C(NCC1CC1)c1nn(-c2cccc(Cl)c2)c2c1CCN(c1ccc3c(n1)CNCC3)C2=O ; C=O ; CN1CCc2ccc(N3CCc4c(C(=O)NCC5CC5)nn(-c5cccc(Cl)c5)c4C3=O)nc2C1]

To a solution of 1-(3-chlorophenyl)-N-(cyclopropylmethyl)-7-oxo-6-(5,6,7,8-tetrahydro-1,7-naphthyridin-2-yl)-4,5-dihydropyrazolo[3,4-c]pyridine-3-carboxamide hydrochloride (40 mg, 0.078 mmol) in a mixture of DCM (1 mL) and MeOH (0.2 mL) were added TEA (33 μL, 0.23 mmol) and formaldehyde, (37 wt. % in H₂O, 29 μL, 0.39 mmol) and the mixture was stirred at rt for 15 min. STAB (25 mg, 0.12 mmol) was added portion-wise and the mixture was stirred for 2 h. The solution was diluted with DCM (50 mL), washed with sat. NaHCO₃(aq) and brine, dried over (Na₂SO₄) and evaporated. The residue was purified by ion-exchange SCX-2 column, washing with MeOH (10 mL) and eluting with 3M NH₃ in MeOH (10 mL) to afford the product (28 mg, 72% yield).

[M+H]⁺=491.1, 493.0

¹H NMR (DMSO-d6, 400 MHZ) δ 0.21-0.27 (2H, m), 0.38-0.46 (2H, m), 1.04 (1H, dddd, J=12.7, 8.1, 5.1, 2.1 Hz), 2.37 (3H, s), 2.61 (2H, t, J=5.8 Hz), 2.80 (2H, t, J=5.8 Hz), 3.12 (2H, t, J=6.5 Hz), 3.16 (2H, t, J=6.9 Hz), 3.50 (2H, s), 4.27 (2H, t, J=6.5 Hz), 7.46 (1H, d, J=8.3 Hz), 7.49-7.57 (3H, m), 7.65 (1H, dt, J=6.8, 2.1 Hz), 7.81-7.84 (1H, m), 8.52 (1H, t, J=6.0 Hz)

### Example Number 1040

### 1-(3-Chlorophenyl)-N-(cyclopropylmethyl)-6-(6-methyl-5,6,7,8-tetrahydro-1,6-naphthyridin-3-yl)-7-oxo-4,5,6,7-tetrahydro-1H-pyrazolo[3,4-c]pyridine-3-carboxamide

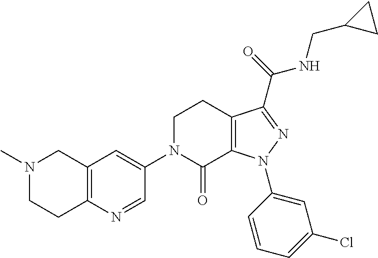 [SMILES: CN1CCc2ncc(N3CCc4c(C(=O)NCC5CC5)nn(-c5cccc(Cl)c5)c4C3=O)cc2C1]

### tert-butyl 3-bromo-7,8-dihydro-5H-1,6-naphthyridine-6-carboxylate

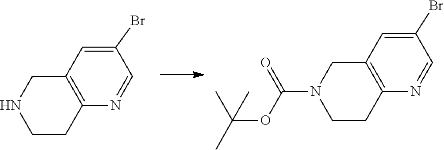 [SMILES: Brc1cnc2c(c1)CNCC2 ; CC(C)(C)OC(=O)N1CCc2ncc(Br)cc2C1]

Boc₂O (262 mg, 1.20 mmol) was added to a solution of 3-bromo-5,6,7,8-tetrahydro-1,6-naphthyridine hydrochloride (250 mg, 1.00 mmol) and TEA (420 μL, 3.01 mmol) in DCM (10 mL). The mixture was stirred at rt for 18 h. Water (10 mL) was added, the organic phase was separated, and the aqueous phase was further extracted with DCM (3×5 mL). The combined organic extracts were washed with brine, dried (Na₂SO₄) and evaporated to afford the product (354 mg, 100% yield).

[M+H]⁺=312.9, 314.9

### tert-Butyl 3-[1-(3-chlorophenyl)-3-ethoxycarbonyl-7-oxo-4,5-dihydropyrazolo[3,4-c]pyridin-6-yl]-7,8-dihydro-5H-1,6-naphthyridine-6-carboxylate

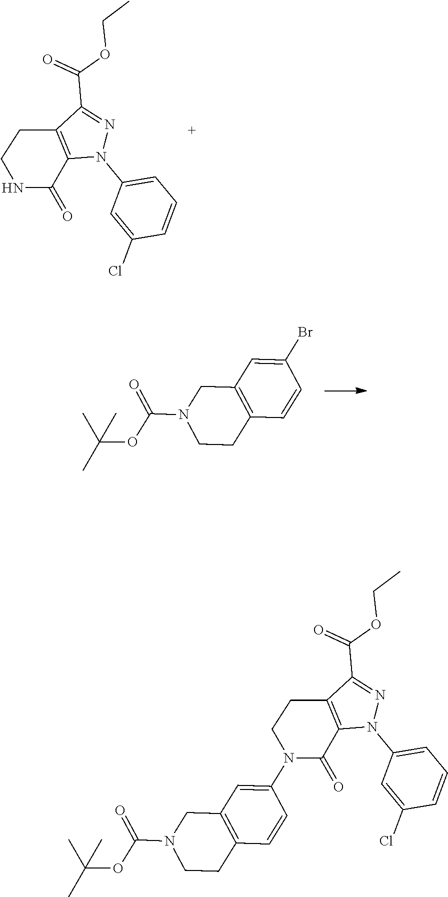 [SMILES: CCOC(=O)c1nn(-c2cccc(Cl)c2)c2c1CCNC2=O ; CC(C)(C)OC(=O)N1CCc2ccc(Br)cc2C1 ; CCOC(=O)c1nn(-c2cccc(Cl)c2)c2c1CCN(c1ccc3c(c1)CN(C(=O)OC(C)(C)C)CC3)C2=O]

Cu(I) I (9.8 mg, 0.051 mmol) was added to a stirring suspension of ethyl 1-(3-chlorophenyl)-7-oxo-5,6-dihydro-4H-pyrazolo[3,4-c]pyridine-3-carboxylate (82 mg, 0.26 mmol), tert-butyl 3-bromo-7,8-dihydro-5H-1,6-naphthyridine-6-carboxylate (117 mg, 0.36 mmol), (1S,2S)—N,N′-dimethyl-1,2-cyclohexane-diamine (15 mg, 0.10 mmol) and K₃PO₄ (136 mg, 0.64 mmol) in anhydrous 1,4-dioxane (1.7 mL). The mixture was then heated at 100° C. for 20 h. The mixture was allowed to cool to rt. The mixture was filtered through Celite®, rinsed with EtOAc (50 mL), the filtrate washed with water and brine, dried (Na₂SO₄) and evaporated. The residue was purified by flash chromatography (silica, 0-100% EtOAc in iso-hexane) to afford the product (100 mg, 44% yield).

[M+H]⁺=552.1, 554.0

¹H NMR (CDCl₃, 400 MHZ) δ 1.44 (3H, t, J=7.1 Hz), 1.48 (9H, s), 2.99 (2H, s), 3.36 (2H, t, J=6.7 Hz), 3.73 (2H, s), 4.12-4.19 (2H, m), 4.48 (2H, q, J=7.1 Hz), 4.58 (2H, s), 7.31-7.50 (4H, m), 7.61 (1H, t, J=2.0 Hz), 8.40 (1H, d, J=2.5 Hz)

### 6-(6-tert-Butoxycarbonyl-7,8-dihydro-5H-1,6-naphthyridin-3-yl)-1-(3-chlorophenyl)-7-oxo-4,5-dihydropyrazolo[3,4-c]pyridine-3-carboxylic acid

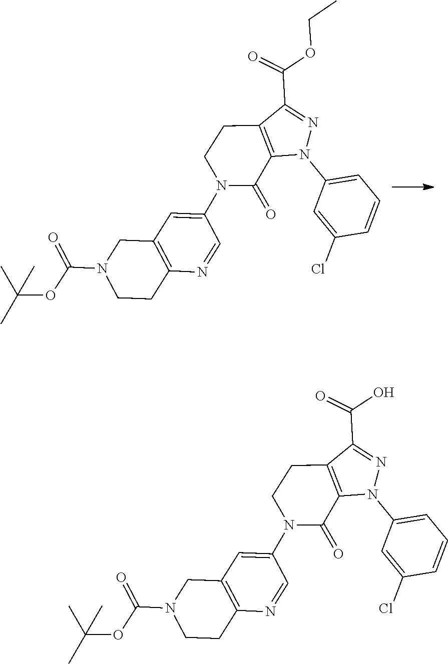 [SMILES: CCOC(=O)c1nn(-c2cccc(Cl)c2)c2c1CCN(c1cnc3c(c1)CN(C(=O)OC(C)(C)C)CC3)C2=O ; CC(C)(C)OC(=O)N1CCc2ncc(N3CCc4c(C(=O)O)nn(-c5cccc(Cl)c5)c4C3=O)cc2C1]

2M NaOH (aq)(0.14 mL, 0.27 mmol) was added to a solution of tert-butyl 3-[1-(3-chlorophenyl)-3-ethoxycarbonyl-7-oxo-4,5-dihydropyrazolo[3,4-c]pyridin-6-yl]-7,8-dihydro-5H-1,6-naphthyridine-6-carboxylate (100 mg, 0.18 mmol) in a mixture of THF (1 mL) and MeOH. The mixture was stirred at rt for 2 h. The reaction solvent was removed, the residue taken up in 5% citric acid (aq)(5 mL) and extracted into EtOAc (3×5 mL). The combined organic extracts were dried (Na₂SO₄) and evaporated to afford the product (104 mg, 100% yield).

[M+H]⁺=524.1, 526.0

¹H NMR (DMSO-d6, 400 MHZ) δ 1.43 (9H, s), 2.86 (2H, t, J=6.0 Hz), 3.20 (2H, t, J=6.6 Hz), 3.66 (2H, t, J=5.9 Hz), 4.00-4.13 (2H, m), 4.54 (2H, s), 7.48-7.67 (4H, m), 7.74 (1H, d, J=2.1 Hz), 8.41 (1H, d, J=2.5 Hz), 13.35 (1H, s)

### tert-Butyl 3-[1-(3-chlorophenyl)-3-(cyclopropylmethylcarbamoyl)-7-oxo-4,5-dihydropyrazolo[3,4-c]pyridin-6-yl]-7,8-dihydro-5H-1,6-naphthyridine-6-carboxylate

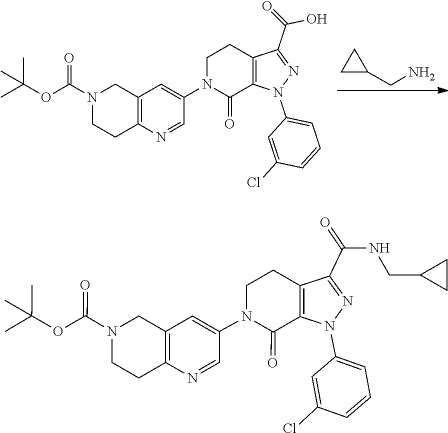 [SMILES: CC(C)(C)OC(=O)N1CCc2ncc(N3CCc4c(C(=O)O)nn(-c5cccc(Cl)c5)c4C3=O)cc2C1 ; NCC1CC1 ; CC(C)(C)OC(=O)N1CCc2ncc(N3CCc4c(C(=O)NCC5CC5)nn(-c5cccc(Cl)c5)c4C3=O)cc2C1]

TCFH (100 mg, 0.36 mmol) was added to a solution of 6-(6-tert-butoxycarbonyl-7,8-dihydro-5H-1,6-naphthyridin-3-yl)-1-(3-chlorophenyl)-7-oxo-4,5-dihydropyrazolo[3,4-c]pyridine-3-carboxylic acid (104 mg, 0.20 mmol), aminomethylcyclopropane (0.022 mL, 0.26 mmol) and 1-methylimidazole (100 μL, 0.89 mmol) in MeCN (2 mL) at 0° C. The mixture was stirred at rt for 20 h, diluted with EtOAc (75 mL), washed with sat. NaHCO₃(aq)(20 mL) and brine (2×20 mL), dried (Na₂SO₄) and evaporated. The residue was purified by flash chromatography (silica, 5-100% EtOAc in iso-hexane) to afford the product (91 mg, 73% yield).

[M+H]⁺=577.2, 579.2

¹H NMR (CDCl₃, 400 MHZ) δ 0.30 (2H, t, J=4.9 Hz), 0.53-0.62 (2H, m), 1.07 (1H, s), 1.48 (9H, s), 2.99 (2H, s), 3.28-3.36 (2H, m), 3.43 (2H, t, J=6.7 Hz), 3.73 (2H, s), 4.13 (2H, t, J=7.0 Hz), 4.58 (2H, s), 7.07 (1H, s), 7.31-7.51 (4H, m), 8.40 (1H, d, J=2.4 Hz)

### 1-(3-Chlorophenyl)-N-(cyclopropylmethyl)-7-oxo-6-(5,6,7,8-tetrahydro-1,6-naphthyridin-3-yl)-4,5-dihydropyrazolo[3,4-c]pyridine-3-carboxamide

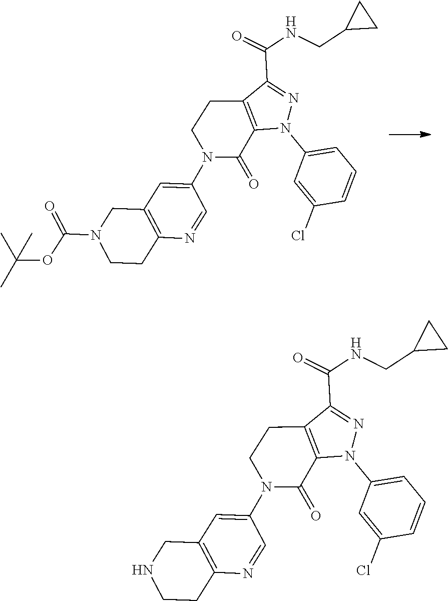 [SMILES: CC(C)(C)OC(=O)N1CCc2ncc(N3CCc4c(C(=O)NCC5CC5)nn(-c5cccc(Cl)c5)c4C3=O)cc2C1 ; O=C(NCC1CC1)c1nn(-c2cccc(Cl)c2)c2c1CCN(c1cnc3c(c1)CNCC3)C2=O]

tert-Butyl 3-[1-(3-chlorophenyl)-3-(cyclopropylmethylcarbamoyl)-7-oxo-4,5-dihydropyrazolo[3,4-c]pyridin-6-yl]-7,8-dihydro-5H-1,6-naphthyridine-6-carboxylate (91 mg, 0.15 mmol) was dissolved in 4M HCl in 1,4-dioxane (3 mL). After 20 h at rt the solvent was evaporated. The residue was purified by SCX ion-exchange, loaded with MeOH (10 mL) and eluted with 2M NH₃ in MeOH (10 mL) to afford the product (64 mg, 92% yield).

[M+H]⁺=477.2

¹H NMR (Methanol-d4, 400 MHz) δ 0.27-0.34 (2H, m), 0.50-0.57 (2H, m), 1.07-1.20 (1H, m), 2.94 (2H, t, J=6.1 Hz), 3.19 (2H, t, J=6.1 Hz), 3.22-3.28 (2H, m), 3.33-3.36 (2H, m), 4.00 (2H, s), 4.15 (2H, t, J=6.6 Hz), 7.44-7.49 (2H, m), 7.58-7.63 (2H, m), 7.72-7.76 (1H, m), 8.40 (1H, d, J=2.4 Hz)

### 1-(3-Chlorophenyl)-N-(cyclopropylmethyl)-6-(6-methyl-5,6,7,8-tetrahydro-1,6-naphthyridin-3-yl)-7-oxo-4,5,6,7-tetrahydro-1H-pyrazolo[3,4-c]pyridine-3-carboxamide

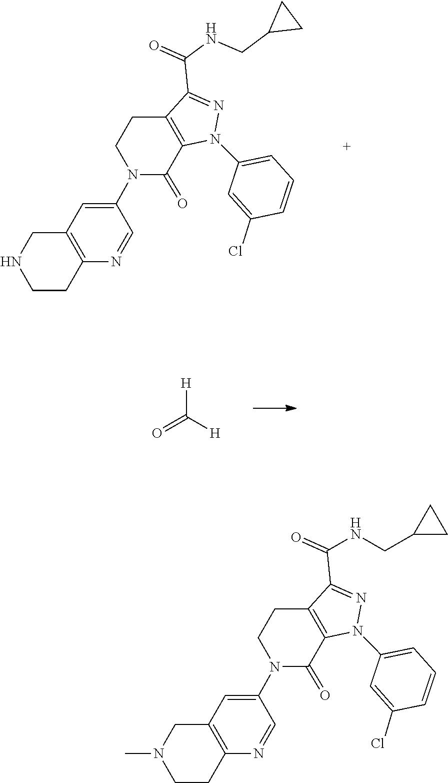 [SMILES: O=C(NCC1CC1)c1nn(-c2cccc(Cl)c2)c2c1CCN(c1cnc3c(c1)CNCC3)C2=O ; C=O ; CN1CCc2ncc(N3CCc4c(C(=O)NCC5CC5)nn(-c5cccc(Cl)c5)c4C3=O)cc2C1]

Formaldehyde, (37 wt. % in H₂O, 50 μL, 0.67 mmol) was added to a solution of 1-(3-chlorophenyl)-N-(cyclopropylmethyl)-7-oxo-6-(5,6,7,8-tetrahydro-1,6-naphthyridin-3-yl)-4,5-dihydropyrazolo[3,4-c]pyridine-3-carboxamide (64 mg, 0.13 mmol) and AcOH (38 μL, 0.67 mmol) in a mixture of DCM (1.5 mL) and MeOH (0.3 mL). The mixture was stirred at rt for 30 min. STAB (57 mg, 0.29 mmol) was added and the resulting mixture was stirred at rt for 1 h. Sat. K₂CO₃ (aq)(5 mL) was added and the mixture was extracted into DCM (3×5 mL). The combined organic extracts were washed with brine (10 mL) and dried (Na₂SO₄), and evaporated. The residue was purified by flash chromatography (silica, 0-15% MeOH in DCM) to afford the product, (33 mg, 50% yield).

[M+H]⁺=491.2, 493.2

¹H NMR (DMSO-d6, 400 MHZ) δ 0.20-0.28 (2H, m), 0.36-0.46 (2H, m), 0.97-1.12 (1H, m), 2.36 (3H, s), 2.69 (2H, t, J=6.0 Hz), 2.88 (2H, t, J=6.0 Hz), 3.12 (2H, t, J=6.4 Hz), 3.22 (2H, t, J=6.6 Hz), 3.50 (2H, s), 4.07 (2H, t, J=6.6 Hz), 7.47-7.58 (3H, m), 7.58-7.67 (1H, m), 7.79 (1H, t, J=1.7 Hz), 8.37 (1H, d, J=2.5 Hz), 8.53 (1H, t, J=6.1 Hz)

### Example Number 1048

### 1-(3-Chlorophenyl)-N-(cyclopropylmethyl)-6-(2,5-dimethyl-1,2,3,4-tetrahydroisoquinolin-7-yl)-7-oxo-4,5,6,7-tetrahydro-1H-pyrazolo[3,4-c]pyridine-3-carboxamide

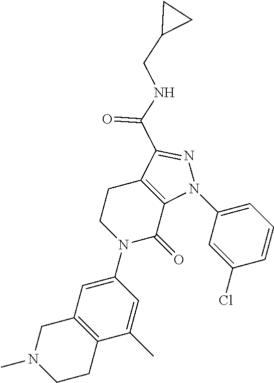 [SMILES: Cc1cc(N2CCc3c(C(=O)NCC4CC4)nn(-c4cccc(Cl)c4)c3C2=O)cc2c1CCN(C)C2]

### tert-Butyl 7-[1-(3-chlorophenyl)-3-ethoxycarbonyl-7-oxo-4,5-dihydropyrazolo[3,4-c]pyridin-6-yl]-5-methyl-3,4-dihydro-1H-isoquinoline-2-carboxylate

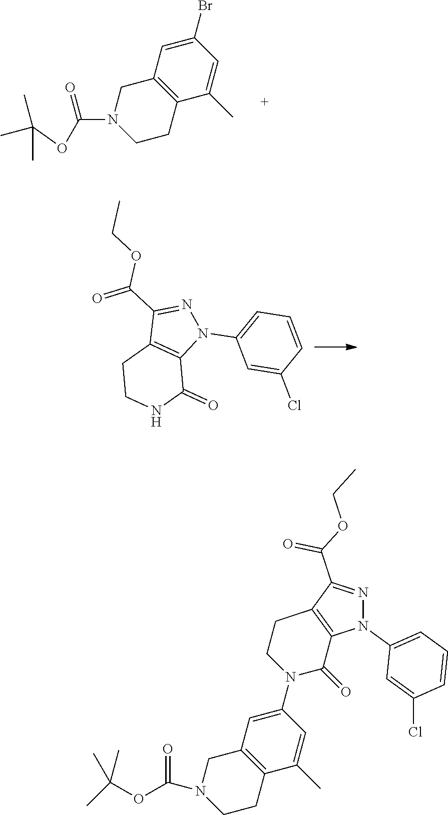 [SMILES: Cc1cc(Br)cc2c1CCN(C(=O)OC(C)(C)C)C2 ; CCOC(=O)c1nn(-c2cccc(Cl)c2)c2c1CCNC2=O ; CCOC(=O)c1nn(-c2cccc(Cl)c2)c2c1CCN(c1cc(C)c3c(c1)CN(C(=O)OC(C)(C)C)CC3)C2=O]

A solution of ethyl 1-(3-chlorophenyl)-7-oxo-5,6-dihydro-4H-pyrazolo[3,4-c]pyridine-3-carboxylate (100 mg, 0.313 mmol) and tert-butyl 7-bromo-5-methyl-3,4-dihydro-1H-isoquinoline-2-carboxylate (122 mg, 0.375 mmol) in 1,4-dioxane (3 mL) was bubbled with N₂ (g) for 5 min. After this time, and maintaining the bubbling, were sequentially added K₃PO₄ (166 mg, 0.782 mmol), (1S,2S)—N,N′-dimethyl-1,2-cyclohexanediamine (18 mg, 0.13 mmol) and finally Cu(I)I (12 mg, 0.063 mmol). After bubbling with N₂ (g) for 2 min the system was closed and stirred at 100° C. for 18 h. The solution was filtered through Celite®, rinsed with EtOAc (50 mL); the filtrate was washed with sat. NaHCO₃(aq) and brine, dried (Na₂SO₄), filtered and evaporated. Flash chromatography (silica, 0-50% EtOAc in iso-hexane) afforded the product (72 mg, 41% yield).

[M−tBu+H]⁺=509.1/511.1

¹H NMR (DMSO-d6, 400 MHZ) δ 1.33 (3H, t, J=7.1 Hz), 1.41 (9H, s), 2.19 (3H, s), 2.64 (2H, t, J=5.9 Hz), 3.18 (2H, t, J=6.6 Hz), 3.58 (2H, t, J=6.1 Hz), 4.00-4.09 (2H, m), 4.35 (2H, q, J=7.1 Hz), 4.47 (2H, s), 6.99 (1H, s), 7.04 (1H, s), 7.48-7.62 (3H, m), 7.71-7.75 (1H, m)

### 6-(2-tert-Butoxycarbonyl-5-methyl-3,4-dihydro-1H-isoquinolin-7-yl)-1-(3-chlorophenyl)-7-oxo-4,5-dihydropyrazolo[3,4-c]pyridine-3-carboxylic acid

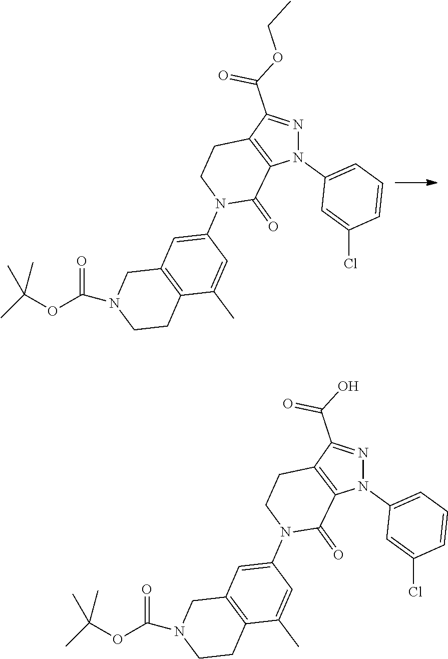 [SMILES: CCOC(=O)c1nn(-c2cccc(Cl)c2)c2c1CCN(c1cc(C)c3c(c1)CN(C(=O)OC(C)(C)C)CC3)C2=O ; Cc1cc(N2CCc3c(C(=O)O)nn(-c4cccc(Cl)c4)c3C2=O)cc2c1CCN(C(=O)OC(C)(C)C)C2]

To a solution of tert-butyl 7-[1-(3-chlorophenyl)-3-ethoxycarbonyl-7-oxo-4,5-dihydropyrazolo[3,4-c]pyridin-6-yl]-5-methyl-3,4-dihydro-1H-isoquinoline-2-carboxylate (70 mg, 0.124 mmol) in a mixture of THF (2 mL) and water (1 mL) was added LiOH·H₂O (13 mg, 0.31 mmol) and the mixture stirred at rt for 18 h. The THF was evaporated and the remaining aqueous solution was acidified to pH=2-3 using 1N HCl (aq). The acidified aqueous phase was extracted with EtOAc and the combined organic phases were dried (Na₂SO₄), filtered and evaporated to provide the product (68 mg).

[M−tBu+H]⁺=481.0/483.0

¹H NMR (DMSO-d6, 400 MHZ) δ 1.42 (9H, s), 2.19 (3H, s), 2.65 (2H, t, J=6.1 Hz), 3.17 (2H, t, J=6.6 Hz), 3.58 (2H, t, J=6.0 Hz), 4.03 (2H, t, J=6.6 Hz), 4.47 (2H, s), 6.99 (1H, s), 7.04 (1H, s), 7.48-7.62 (3H, m), 7.70-7.73 (1H, m), 13.32 (1H, s br)

### tert-Butyl 7-[1-(3-chlorophenyl)-3-(cyclopropylmethylcarbamoyl)-7-oxo-4,5-dihydropyrazolo[3,4-c]pyridin-6-yl]-5-methyl-3,4-dihydro-1H-isoquinoline-2-carboxylate

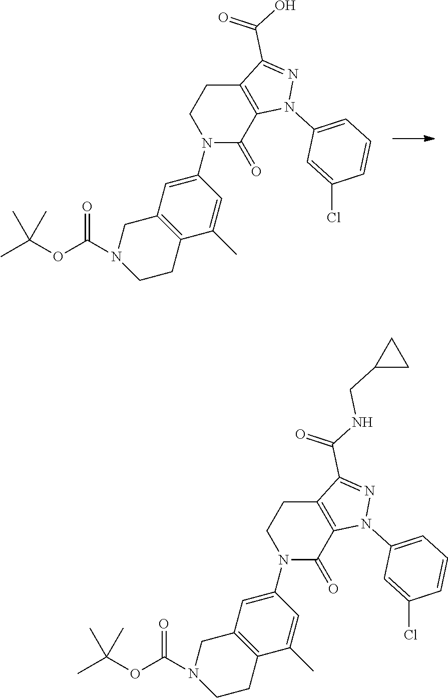 [SMILES: Cc1cc(N2CCc3c(C(=O)O)nn(-c4cccc(Cl)c4)c3C2=O)cc2c1CCN(C(=O)OC(C)(C)C)C2 ; Cc1cc(N2CCc3c(C(=O)NCC4CC4)nn(-c4cccc(Cl)c4)c3C2=O)cc2c1CCN(C(=O)OC(C)(C)C)C2]

To a suspension of 6-(2-tert-butoxycarbonyl-5-methyl-3,4-dihydro-1H-isoquinolin-7-yl)-1-(3-chlorophenyl)-7-oxo-4,5-dihydropyrazolo[3,4-c]pyridine-3-carboxylic acid (65 mg, 0.121 mmol) in MeCN (1.5 mL) were added aminomethylcyclopropane (0.015 mL, 0.17 mmol), 1-methylimidazole (0.034 mL, 0.42 mmol) and TCFH (44 mg, 0.16 mmol), and the mixture was stirred for 20 h at rt. The solution was diluted with EtOAc (50 mL) and washed with sat. NaHCO₃(aq) and brine, dried (Na₂SO₄), filtered and evaporated. Flash chromatography (silica, 2-50% EtOAc in iso-hexane) afforded the product (52 mg, 73% yield).

[M−tBu+H]⁺=534.2/536.2

¹H NMR (DMSO-d6, 400 MHZ) δ 0.21-0.28 (2H, m), 0.38-0.46 (2H, m), 1.00-1.10 (1H, m), 1.42 (9H, s), 2.19 (3H, s), 2.64 (2H, t, J=6.0 Hz), 3.12 (2H, t, J=6.4 Hz), 3.19 (2H, t, J=6.6 Hz), 3.58 (2H, t, J=5.9 Hz), 4.02 (2H, t, J=6.9 Hz), 4.47 (2H, s), 7.00 (1H, s), 7.04 (1H, s), 7.48-7.57 (2H, m), 7.60-7.66 (1H, m), 7.75 -7.79 (1H, m), 8.51 (1H, t, J=6.0 Hz)

### 1-(3-Chlorophenyl)-N-(cyclopropylmethyl)-6-(5-methyl-1,2,3,4-tetrahydroisoquinolin-7-yl)-7-oxo-4,5-dihydropyrazolo[3,4-c]pyridine-3-carboxamide hydrochloride

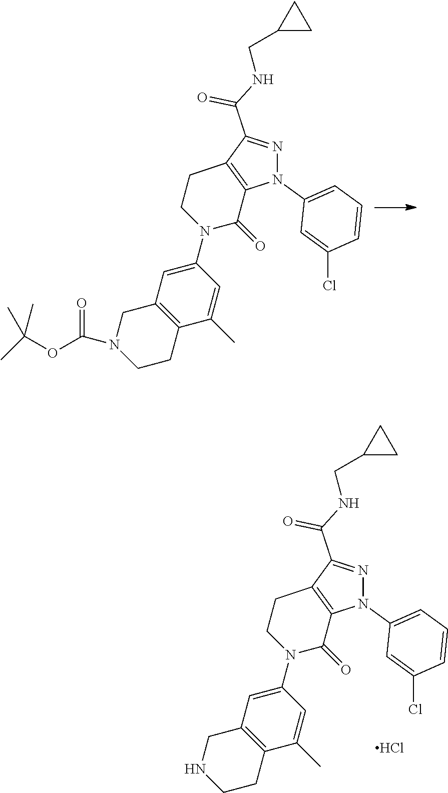 [SMILES: Cc1cc(N2CCc3c(C(=O)NCC4CC4)nn(-c4cccc(Cl)c4)c3C2=O)cc2c1CCN(C(=O)OC(C)(C)C)C2 ; Cc1cc(N2CCc3c(C(=O)NCC4CC4)nn(-c4cccc(Cl)c4)c3C2=O)cc2c1CCNC2 ; Cl]

To a solution of tert-butyl 7-[1-(3-chlorophenyl)-3-(cyclopropylmethylcarbamoyl)-7-oxo-4,5-dihydropyrazolo[3,4-c]pyridin-6-yl]-5-methyl-3,4-dihydro-1H-isoquinoline-2-carboxylate (50 mg, 0.085 mmol) in 1,4-dioxane (0.5 mL) was added HCl (4M in 1,4-dioxane)(0.44 mL, 1.8 mmol) and the mixture stirred for 20 h at rt. The solvent was evaporated to afford the product (45 mg).

[M+H]⁺=490.1/492.1

¹H NMR (DMSO-d6, 400 MHZ) δ 0.20-0.29 (2H, m), 0.38-0.48 (2H, m), 1.00-1.11 (1H, m), 2.22 (3H, s), 2.84 (2H, t, J=6.3 Hz), 3.12 (2H, t, J=6.4 Hz), 3.21 (2H, t, J=6.6 Hz), 3.40 (2H, t, J=8.1 Hz), 4.02 (2H, t, J=6.6 Hz), 4.23 (2H, s), 7.08 (1H, s), 7.16 (1H, s), 7.47-7.58 (2H, m), 7.62 (1H, dt, J=6.9, 2.0 Hz), 7.74-7.79 (1H, m), 8.52 (1H, t, J=6.1 Hz), 9.16 (2H, s br)

### 1-(3-Chlorophenyl)-N-(cyclopropylmethyl)-6-(2,5-dimethyl-1,2,3,4-tetrahydroisoquinolin-7-yl)-7-oxo-4,5,6,7-tetrahydro-1H-pyrazolo[3,4-c]pyridine-3-carboxamide

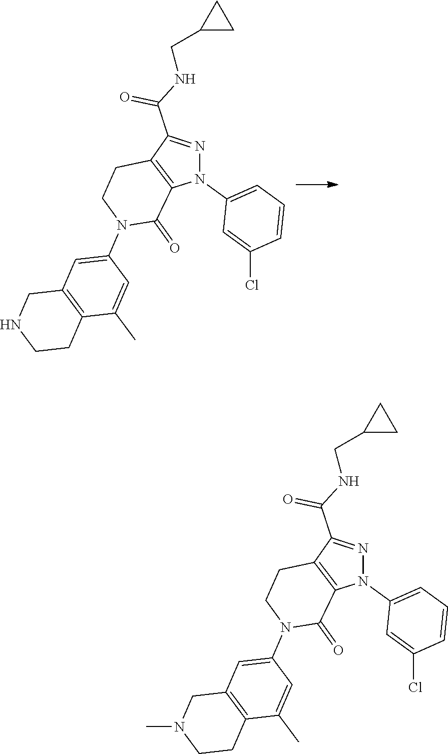 [SMILES: Cc1cc(N2CCc3c(C(=O)NCC4CC4)nn(-c4cccc(Cl)c4)c3C2=O)cc2c1CCNC2 ; Cc1cc(N2CCc3c(C(=O)NCC4CC4)nn(-c4cccc(Cl)c4)c3C2=O)cc2c1CCN(C)C2]

To a solution of 1-(3-chlorophenyl)-N-(cyclopropylmethyl)-6-(5-methyl-1,2,3,4-tetrahydroisoquinolin-7-yl)-7-oxo-4,5-dihydropyrazolo[3,4-c]pyridine-3-carboxamide hydrochloride (43 mg, 0.082 mmol) in a mixture of DCM (1 mL) and MeOH (0.2 mL) were added TEA (0.034 mL, 0.25 mmol) and formaldehyde (37 wt. % in H₂O)(0.030 mL, 0.41 mmol) and the mixture was stirred at rt for 30 min. STAB (26 mg, 0.12 mmol) was added portion-wise and the mixture was stirred for 72 h. The solution was diluted with DCM (50 mL) and washed with sat. NaHCO₃(aq) and brine, dried (Na₂SO₄), filtered and evaporated. The product was purified by loading onto an ion-exchange SCX-2 column, washing with MeOH and eluting with 3M NH₃ in MeOH. The eluent was concentrated and lyophilised to provide the product (32 mg, 75% yield).

[M+H]⁺=504.1/506.1

¹H NMR (DMSO-d6, 400 MHZ) δ 0.19-0.28 (2H, m), 0.35-0.46 (2H, m), 0.97-1.12 (1H, m), 2.16 (3H, s), 2.32 (3H, s), 2.59-2.68 (4H, m), 3.11 (2H, t, J=6.5 Hz), 3.18 (2H, t, J=6.7 Hz), 3.43 (2H, s), 4.00 (2H, t, J=6.6 Hz), 6.84-6.89 (1H, m), 6.96-7.01 (1H, m), 7.45-7.56 (2H, m), 7.56-7.65 (1H, m), 7.74-7.79 (1H, m), 8.52 (1H, t, J=6.0 Hz)

### Example Number 1054

### 1-(3-Chlorophenyl)-N-(cyclopropylmethyl)-6-(2-ethyl-6-methyl-1,2,3,4-tetrahydroisoquinolin-7-yl)-7-oxo-4,5,6,7-tetrahydro-1H-pyrazolo[3,4-c]pyridine-3-carboxamide

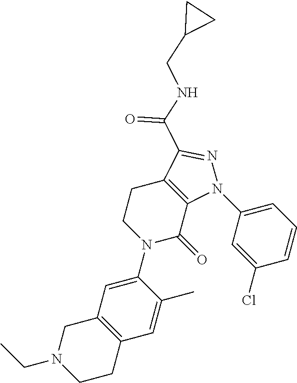 [SMILES: CCN1CCc2cc(C)c(N3CCc4c(C(=O)NCC5CC5)nn(-c5cccc(Cl)c5)c4C3=O)cc2C1]

### tert-Butyl 7-bromo-6-methyl-3,4-dihydro-1H-isoquinoline-2-carboxylate

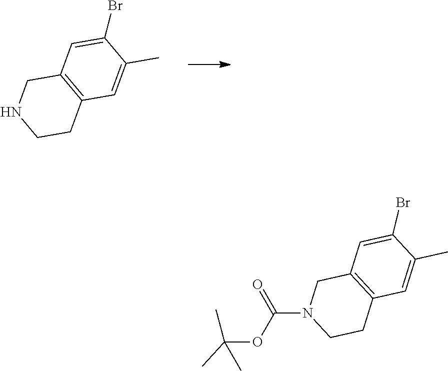 [SMILES: Cc1cc2c(cc1Br)CNCC2 ; Cc1cc2c(cc1Br)CN(C(=O)OC(C)(C)C)CC2]

Boc₂O (157 mg, 0.719 mmol) was added to a stirring solution of 7-bromo-6-methyl-1,2,3,4-tetrahydroisoquinoline (125 mg, 0.553 mmol) and TEA (0.15 mL, 1.11 mmol) in DCM (5 mL) at rt. The mixture was stirred at rt for 20 h. Water (5 mL) was added, the organic phase was separated and the aqueous phase extracted into DCM (3×5 mL). The organic fractions were combined, washed with sat. NaHCO₃(aq)(10 mL) and sat. brine solution (10 mL), dried (Na₂SO₄), filtered and concentrated to give the product (199 mg, 100% yield), which was used in subsequent reactions without further purification.

¹H NMR (CDCl₃, 400 MHZ) δ 1.48 (9H, s), 2.34 (3H, d, J=0.9 Hz), 2.74 (2H, t, J=5.9 Hz), 3.61 (2H, s), 4.50 (2H, s), 7.00 (1H, s), 7.28 (1H, s) tert-Butyl 7-[1-(3-chlorophenyl)-3-ethoxycarbonyl-7-oxo-4,5-dihydropyrazolo[3,4-c]pyridin-6-yl]-6-methyl-3,4-dihydro-1H-isoquinoline-2-carboxylate

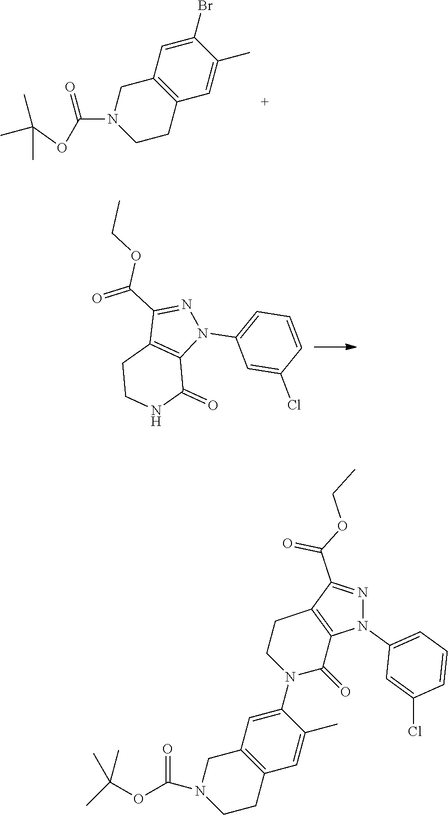 [SMILES: Cc1cc2c(cc1Br)CN(C(=O)OC(C)(C)C)CC2 ; CCOC(=O)c1nn(-c2cccc(Cl)c2)c2c1CCNC2=O ; CCOC(=O)c1nn(-c2cccc(Cl)c2)c2c1CCN(c1cc3c(cc1C)CCN(C(=O)OC(C)(C)C)C3)C2=O]

Cu(I)I (19 mg, 0.10 mmol) was added to a suspension of ethyl 1-(3-chlorophenyl)-7-oxo-5,6-dihydro-4H-pyrazolo[3,4-c]pyridine-3-carboxylate (160 mg, 0.500 mmol), tert-butyl 7-bromo-6-methyl-3,4-dihydro-1H-isoquinoline-2-carboxylate (196 mg, 0.600 mmol), (1S,2S)—N,N′-dimethyl-1,2-cyclohexane-diamine (28 mg, 0.20 mmol) and K₃PO₄ (266 mg, 1.25 mmol) in anhydrous 1,4-dioxane (3.5 mL) at rt. The mixture was sparged with N₂ (g) for 5 min, heated to 100° C. and stirred for 20 h. The mixture was filtered through a pad of Celite®, and the organics were washed through with EtOAc (5×10 mL). The organic fractions were combined, washed with water (20 mL) and sat. brine solution (20 mL), dried (Na₂SO₄), filtered and concentrated. The product was purified by chromatography (silica, 5-100% EtOAc in iso-hexane) to yield the product (56 mg, 20% yield).

[M−tBu+H]⁺=509.2/511.1

### 6-(2-tert-Butoxycarbonyl-6-methyl-3,4-dihydro-1H-isoquinolin-7-yl)-1-(3-chlorophenyl)-7-oxo-4,5-dihydropyrazolo[3,4-c]pyridine-3-carboxylic acid

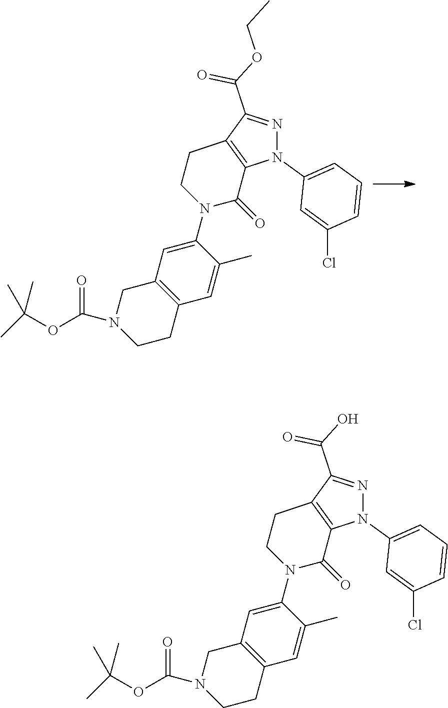 [SMILES: CCOC(=O)c1nn(-c2cccc(Cl)c2)c2c1CCN(c1cc3c(cc1C)CCN(C(=O)OC(C)(C)C)C3)C2=O ; Cc1cc2c(cc1N1CCc3c(C(=O)O)nn(-c4cccc(Cl)c4)c3C1=O)CN(C(=O)OC(C)(C)C)CC2]

LiOH·H₂O (18 mg, 0.43 mmol) was added to a solution of tert-butyl 7-[1-(3-chlorophenyl)-3-ethoxycarbonyl-7-oxo-4,5-dihydropyrazolo[3,4-c]pyridin-6-yl]-6-methyl-3,4-dihydro-1H-isoquinoline-2-carboxylate (96 mg, 0.170 mmol) in a mixture of THF (1 mL) and water (0.5 mL) at rt. The mixture was stirred at rt for 6 h. The solvent was removed, 5% citric acid (aq)(5 ml) was added and the organics were extracted into EtOAc (3×5 mL). Extracts were combined, washed with saturated brine solution (10 mL), dried (Na₂SO₄), filtered and concentrated to give the product (80 mg, 88% yield), which was carried directly into the next reaction without further processing.

### tert-Butyl 7-[1-(3-chlorophenyl)-3-(cyclopropylmethylcarbamoyl)-7-oxo-4,5-dihydropyrazolo[3,4-c]pyridin-6-yl]-6-methyl-3,4-dihydro-1H-isoquinoline-2-carboxylate

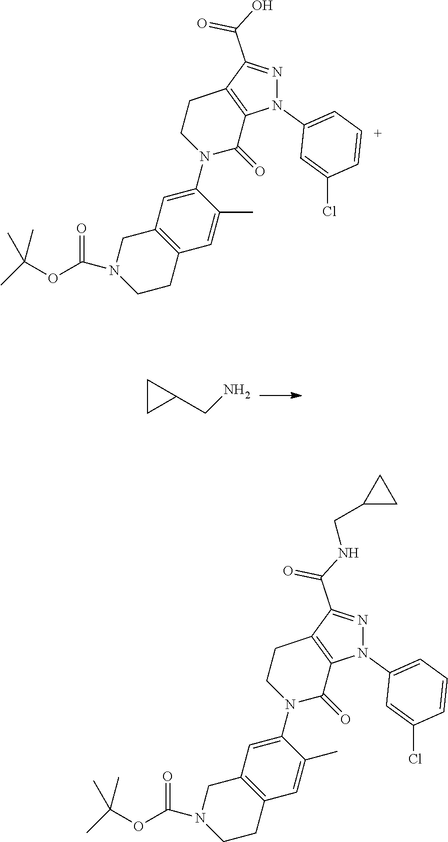 [SMILES: Cc1cc2c(cc1N1CCc3c(C(=O)O)nn(-c4cccc(Cl)c4)c3C1=O)CN(C(=O)OC(C)(C)C)CC2 ; NCC1CC1 ; Cc1cc2c(cc1N1CCc3c(C(=O)NCC4CC4)nn(-c4cccc(Cl)c4)c3C1=O)CN(C(=O)OC(C)(C)C)CC2]

TCFH (63 mg, 0.22 mmol) was added to a stirred suspension of 6-(2-tert-butoxycarbonyl-6-methyl-3,4-dihydro-1H-isoquinolin-7-yl)-1-(3-chlorophenyl)-7-oxo-4,5-dihydropyrazolo[3,4-c]pyridine-3-carboxylic acid (80 mg, 0.15 mmol), aminomethylcyclopropane (0.017 mL, 0.19 mmol) and 1-methylimidazole (0.053 mL, 0.67 mmol) in anhydrous MeCN (2 mL) at rt. The mixture was stirred at rt for 20 h. After removal of solvent, water (5 mL) was added and extracted with EtOAc (3×5 mL). The extracts were combined, washed with sat. NaHCO₃(aq)(10 mL) and saturated brine solution (10 mL), dried (Na₂SO₄), filtered and concentrated to give the product. This was purified by flash chromatography (silica, 0-80% EtOAc in iso-hexane) to yield the product (68 mg, 77 yield).

[M−tBu+H]⁺=534.1/536.1

¹H NMR (CDCl₃, 400 MHZ) δ 0.25-0.34 (2H, m), 0.53-0.62 (2H, m), 1.03-1.11 (1H, m), 1.48 (9H, s), 2.20 (3H, s), 2.78 (2H, s), 3.30-3.41 (2H, m), 3.38-3.49 (2H, m), 3.62-3.67 (3H, m), 4.49 (2H, s), 6.93 (1H, s), 7.02-7.10 (2H, m), 7.22 (1H, s), 7.31-7.40 (2H, m), 7.50-7.57 (1H, m), 7.62-7.67 (1H, m)

### 1-(3-Chlorophenyl)-N-(cyclopropylmethyl)-6-(6-methyl-1,2,3,4-tetrahydroisoquinolin-7-yl)-7-oxo-4,5-dihydropyrazolo[3,4-c]pyridine-3-carboxamide hydrochloride

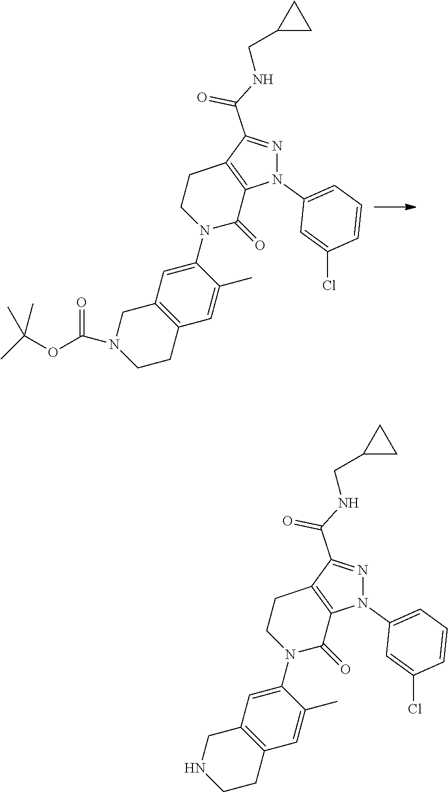 [SMILES: Cc1cc2c(cc1N1CCc3c(C(=O)NCC4CC4)nn(-c4cccc(Cl)c4)c3C1=O)CN(C(=O)OC(C)(C)C)CC2 ; Cc1cc2c(cc1N1CCc3c(C(=O)NCC4CC4)nn(-c4cccc(Cl)c4)c3C1=O)CNCC2]

HCl (4N in 1,4-dioxane)(0.50 mL) was added to a solution of tert-butyl 7-[1-(3-chlorophenyl)-3-(cyclopropylmethylcarbamoyl)-7-oxo-4,5-dihydropyrazolo[3,4-c]pyridin-6-yl]-6-methyl-3,4-dihydro-1H-isoquinoline-2-carboxylate (68 mg, 0.115 mmol) in 1,4-dioxane (0.5 mL) at rt. The mixture was stirred at rt for 20 h. Evaporation afforded the product (61 mg, 99% yield) which was used in subsequent reactions without further purification.

[M+H]⁺=490.2/492.2

¹H NMR (Methanol-d4, 400 MHZ) δ 0.30 (2H, dt, J=6.0, 4.5 Hz), 0.49-0.58 (2H, m), 1.06-1.18 (1H, m), 2.25 (3H, s), 3.10 (2H, t, J=6.4 Hz), 3.22-3.29 (2H, m), 3.31-3.56 (4H, m), 3.82-3.93 (1H, m), 4.04-4.16 (1H, m), 4.32 (2H, s), 7.17 (1H, s), 7.22 (1H, s), 7.43-7.50 (2H, m), 7.55-7.64 (1H, m), 7.73 (1H, s)

### 1-(3-Chlorophenyl)-N-(cyclopropylmethyl)-6-(2-ethyl-6-methyl-1,2,3,4-tetrahydroisoquinolin-7-yl)-7

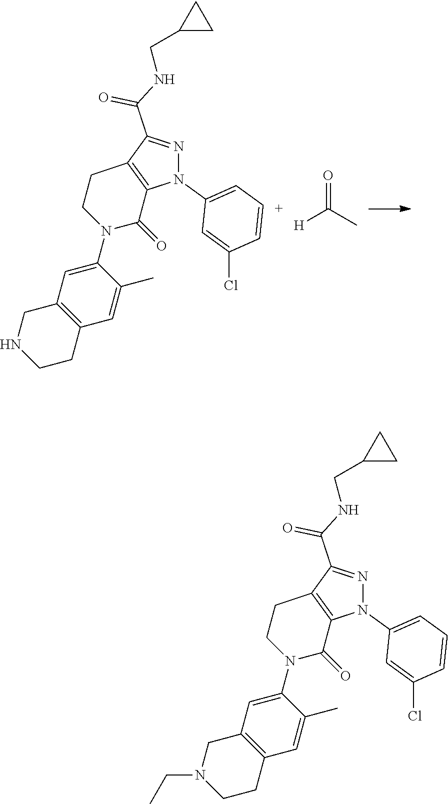

Acetaldehyde (50 wt. % solution in EtOH)(0.046 mL, 0.46 mmol) was added to a solution of 1-(3-chlorophenyl)-N-(cyclopropylmethyl)-6-(6-methyl-1,2,3,4-tetrahydroisoquinolin-7-yl)-7-oxo-4,5-dihydropyrazolo[3,4-c]pyridine-3-carboxamide hydrochloride (61 mg, 0.115 mmol) and TEA (0.064 mL, 0.46 mmol) in a mixture of DCM (1.5 mL) and MeOH (0.3 mL) at rt. The mixture was stirred at rt for 1 h. STAB (37 mg, 0.17 mmol) was added and the mixture was stirred at rt for 2 h. Sat. K₂CO₃ (aq)(10 mL) was added and the mixture extracted with DCM (3×5 mL). The DCM fractions were combined, washed with sat. brine solution (10 mL), dried (Na₂SO₄), filtered and concentrated to give the product. This was purified by chromatography (silica, 0-15% MeOH in DCM) and lyophilised to give the product (40 mg, 66% yield).

[M+H]⁺=518.2/520.2

¹H NMR (DMSO-d6, 400 MHZ) δ 0.20-0.28 (2H, m), 0.36-0.46 (2H, m), 0.99-1.14 (4H, m), 2.10 (3H, s), 2.47 (2H, t, J=7.1 Hz), 2.55-2.68 (2H, m), 2.76 (2H, t, J=5.9 Hz), 3.12 (2H, t, J=6.5 Hz), 3.13-3.32 (2H, m), 3.42-3.55 (2H, m), 3.68-3.78 (1H, m), 3.97-4.08 (1H, m), 6.99 (2H, d, J=7.4 Hz), 7.46-7.56 (2H, m), 7.57-7.67 (1H, m), 7.74-7.80 (1H, m), 8.52 (1H, t, J=6.0 Hz)

### Example number 1055

### 1-(3-Chlorophenyl)-N-(cyclopropylmethyl)-6-(2-(2-hydroxyethyl)-1,2,3,4-tetrahydroisoquinolin-7-yl)-7

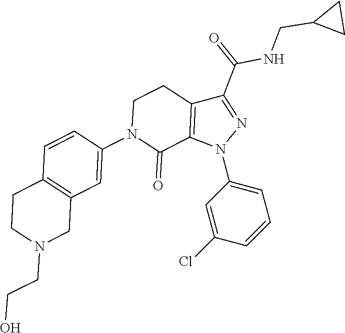 [SMILES: O=C(NCC1CC1)c1nn(-c2cccc(Cl)c2)c2c1CCN(c1ccc3c(c1)CN(CCO)CC3)C2=O]

### 6-(2-tert-Butoxycarbonyl-3,4-dihydro-1H-isoquinolin-7-yl)-1-(3-chlorophenyl)-7-oxo-4,5-dihydropyrazolo[3,4-c]pyridine-3-carboxylic acid

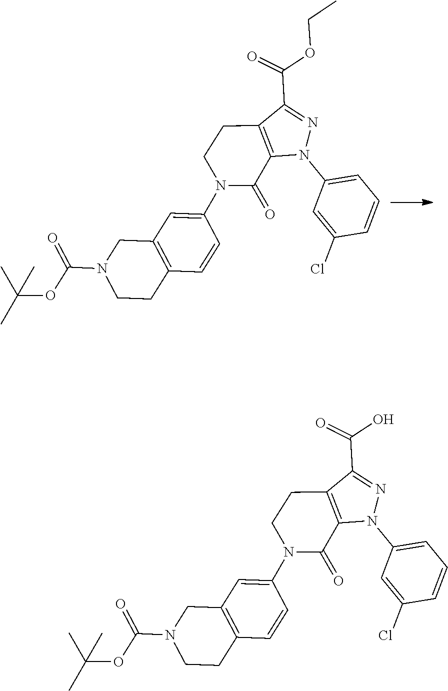 [SMILES: CCOC(=O)c1nn(-c2cccc(Cl)c2)c2c1CCN(c1ccc3c(c1)CN(C(=O)OC(C)(C)C)CC3)C2=O ; CC(C)(C)OC(=O)N1CCc2ccc(N3CCc4c(C(=O)O)nn(-c5cccc(Cl)c5)c4C3=O)cc2C1]

To a solution of tert-butyl 7-[1-(3-chlorophenyl)-3-ethoxycarbonyl-7-oxo-4,5-dihydropyrazolo[3,4-c]pyridin-6-yl]-3,4-dihydro-1H-isoquinoline-2-carboxylate (785 mg, 1.42 mmol) in a mixture of THF (10 mL) and water (5 ml) was added LiOH·H₂O (149 mg, 3.56 mmol). The mixture was stirred at rt for 72 h. The THF was removed and the remaining aqueous solution was acidified to pH=2-3 using 1M HCl (aq). The acidified aqueous phase was extracted with EtOAc (3×40 mL), the combined organic extracts were dried (Na₂SO₄) and evaporated to afford the product (661 mg, 89% yield).

[M−^{t}Bu+H]⁺=467.1, 469.0

### tert-Butyl 7-[1-(3-chlorophenyl)-3-(cyclopropylmethylcarbamoyl)-7-oxo-4,5-dihydropyrazolo[3,4-c]pyridin-6-yl]-3,4-dihydro-1H-isoquinoline-2-carboxylate

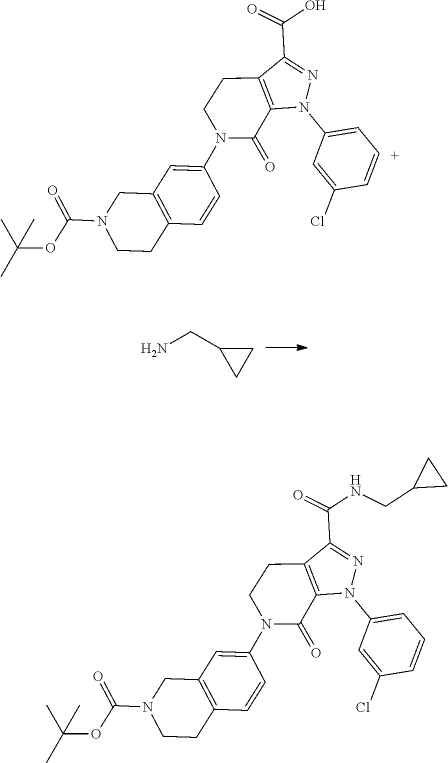 [SMILES: CC(C)(C)OC(=O)N1CCc2ccc(N3CCc4c(C(=O)O)nn(C5=C[C+]=CC(Cl)=C5)c4C3=O)cc2C1 ; NCC1CC1 ; CC(C)(C)OC(=O)N1CCc2ccc(N3CCc4c(C(=O)NCC5CC5)nn(-c5cccc(Cl)c5)c4C3=O)cc2C1]

To a suspension of 6-(2-tert-butoxycarbonyl-3,4-dihydro-1H-isoquinolin-7-yl)-1-(3-chlorophenyl)-7-oxo-4,5-dihydropyrazolo[3,4-c]pyridine-3-carboxylic acid (660 mg, 1.26 mmol) in dry MeCN (12 mL), were added aminomethylcyclopropane (0.15 mL, 1.77 mmol), 1-methylimidazole (0.35 mL, 4.42 mmol) and TCFH (460 mg, 1.64 mmol). The mixture was stirred at rt for 20 h. This solution was diluted with EtOAc (75 mL) and washed with sat. NaHCO₃ (20 mL) and brine (2×20 mL), dried (Na₂SO₄) and evaporated. The residue was purified by flash chromatography (silica, 2-50% EtOAc in iso-hexane) to afford the product (625 mg, 86% yield).

[M−^{t}Bu+H]⁺=520.1, 522.1

¹H NMR (DMSO-d6, 400 MHZ) δ 0.18-0.30 (2H, m), 0.39-0.47 (2H, m), 0.98-1.10 (1H, m), 1.42 (9H, s), 2.76 (2H, t, J=5.9 Hz), 3.12 (2H, t, J=6.5 Hz), 3.20 (2H, t, J=6.6 Hz), 3.54 (2H, t, J=5.9 Hz), 3.99-4.08 (2H, m), 4.48 (2H, s), 7.16-7.19 (3H, m), 7.48-7.55 (2H, m), 7.61-7.65 (1H, m), 7.76-7.79 (1H, m), 8.52 (1H, t, J=6.0 Hz)

### 1-(3-Chlorophenyl)-N-(cyclopropylmethyl)-7-oxo-6-(1,2,3,4-tetrahydroisoquinolin-7-yl)-4,5-dihydropyrazolo[3,4-c]pyridine-3-carboxamide hydrochloride

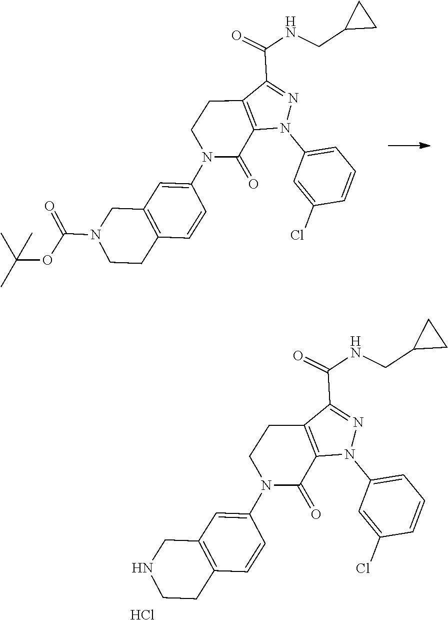 [SMILES: CC(C)(C)OC(=O)N1CCc2ccc(N3CCc4c(C(=O)NCC5CC5)nn(-c5cccc(Cl)c5)c4C3=O)cc2C1 ; O=C(NCC1CC1)c1nn(-c2cccc(Cl)c2)c2c1CCN(c1ccc3c(c1)CNCC3)C2=O ; Cl]

tert-Butyl 7-[1-(3-chlorophenyl)-3-(cyclopropylmethylcarbamoyl)-7-oxo-4,5-dihydropyrazolo[3,4-c]pyridin-6-yl]-3,4-dihydro-1H-isoquinoline-2-carboxylate (620 mg, 1.08 mmol) was dissolved in 4M HCl in 1,4-dioxane (3 mL). After 20 h at rt the solvent was removed to afford the product (563 mg, 98% yield).

[M+H]⁺=476.2, 478.2

¹H NMR (DMSO-d6, 400 MHZ) δ 0.19-0.28 (2H, m), 0.36-0.46 (2H, m), 0.97-1.10 (1H, m), 2.99 (2H, t, J=6.2 Hz), 3.12 (2H, t, J=6.4 Hz), 3.21 (2H, t, J=6.6 Hz), 3.36-3.40 (2H, m), 4.04 (2H, t, J=6.6 Hz), 4.25 (2H, s), 7.22-7.31 (3H, m), 7.47-7.57 (2H, m), 7.57-7.66 (1H, m), 7.75-7.81 (1H, m), 8.54 (1H, t, J=6.0 Hz), 9.17 (2H, s)

### 1-(3-Chlorophenyl)-N-(cyclopropylmethyl)-6-(2-(2-hydroxyethyl)-1,2,3,4-tetrahydroisoquinolin-7-yl)-7

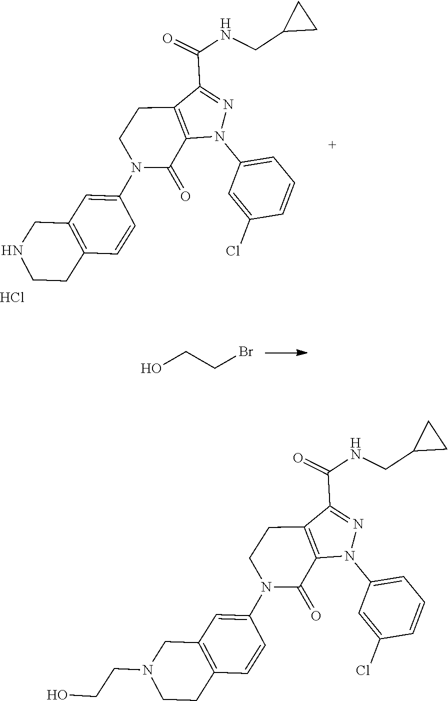

1-(3-Chlorophenyl)-N-(cyclopropylmethyl)-7-oxo-6-(1,2,3,4-tetrahydroisoquinolin-7-yl)-4,5-dihydropyrazolo[3,4-c]pyridine-3-carboxamide hydrochloride (70 mg, 0.14 mmol) was dissolved in DMF (1.5 mL). TEA (67 μL, 0.48 mmol) and 2-bromoethanol (29 μL, 0.41 mmol) were added. The reaction was stirred at rt for 20 h, diluted with EtOAc (100 mL), washed with sat. NaHCO₃ (3×15 mL), brine (3 x mL), dried (Na₂SO₄), filtered and evaporated. The residue was purified by flash chromatography (Silica, 0-10% MeOH in DCM), further purified on-exchange SCX-2 column, washing with MeOH (10 mL) and eluting with 3 M NH₃ in MeOH (10 mL) to afford the product (42 mg, 58% yield).

[M+H]⁺=520.2, 522.2

¹H NMR (DMSO-d6, 400 MHZ) δ 0.21-0.28 (2H, m), 0.38-0.45 (2H, m), 1.00-1.10 (1H, m), 2.55 (2H, t, J=6.2 Hz), 2.69 (2H, t, J=5.8 Hz), 2.78 (2H, t, J=5.7 Hz), 3.12 (2H, t, J=6.5 Hz), 3.19 (2H, t, J=6.6 Hz), 3.54 -3.61 (4H, m), 4.02 (2H, t, J=6.6 Hz), 4.47 (1H, t, J=5.4 Hz), 7.04 (1H, s), 7.11 (2H, s), 7.48-7.55 (2H, m), 7.62 (1H, dt, J=6.8, 2.1 Hz), 7.76-7.80 (1H, m), 8.51 (1H, t, J=6.0 Hz)

### Example Number 1056

### 1-(3-Chlorophenyl)-N-(cyclopropylmethyl)-6-(7-ethyl-5,6,7,8-tetrahydropyrido[3,4-d]pyrimidin-2-yl)-7

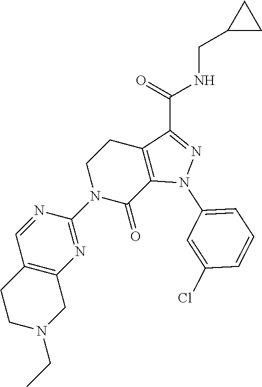 [SMILES: CCN1CCc2cnc(N3CCc4c(C(=O)NCC5CC5)nn(-c5cccc(Cl)c5)c4C3=O)nc2C1]

### tert-Butyl 2-[1-(3-chlorophenyl)-3-ethoxycarbonyl-7-oxo-4,5-dihydropyrazolo[3,4-c]pyridin-6-yl]-6,8-dihydro-5H-pyrido[3,4-d]pyrimidine-7-carboxylate

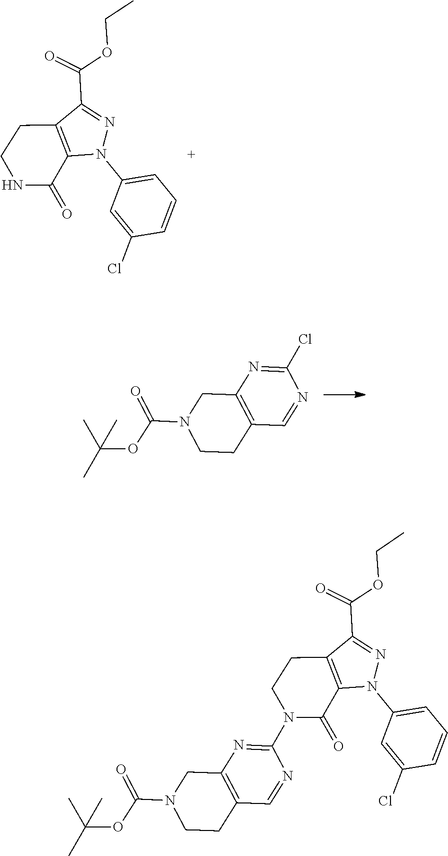 [SMILES: CCOC(=O)c1nn(-c2cccc(Cl)c2)c2c1CCNC2=O ; CC(C)(C)OC(=O)N1CCc2cnc(Cl)nc2C1 ; CCOC(=O)c1nn(-c2cccc(Cl)c2)c2c1CCN(c1ncc3c(n1)CN(C(=O)OC(C)(C)C)CC3)C2=O]

A solution of ethyl 1-(3-chlorophenyl)-7-oxo-5,6-dihydro-4H-pyrazolo[3,4-c]pyridine-3-carboxylate (100 mg, 0.31 mmol) and tert-butyl 2-chloro-5H,6H,7H,8H-pyrido[3,4-d]pyrimidine-7-carboxylate (101 mg, 0.38 mmol) in dry 1,4-dioxane (3 mL) was purged with N₂ (g) for 5 min. Cs₂CO₃ (204 mg, 0.63 mmol), XantPhos (36 mg, 0.063 mmol) and Pd₂(dba)₃ (29 mg, 0.031 mmol) were added. The sealed reaction vessel was stirred at 100° C. for 20 h. The solution was filtered through Celite®, the residue was washed with EtOAc (50 mL), the filtrate was washed with water (15 mL) and brine (2×15 mL), dried (Na₂SO₄) and evaporated. The residue was purified by flash chromatography (silica, 5-80% EtOAc in iso-hexane) to afford the product (98 mg, 57% yield).

[M+H]⁺=553.2, 555.2

¹H NMR (DMSO-d6, 400 MHZ) δ 1.33 (3H, t, J=7.1 Hz), 1.42 (9H, s), 2.79 (2H, t, J=5.7 Hz), 3.18 (2H, t, J=6.4 Hz), 3.61 (2H, t, J=5.8 Hz), 4.24 (2H, t, J=6.4 Hz), 4.35 (2H, q, J=7.1 Hz), 4.48 (2H, s), 7.50-7.64 (3H, m), 7.74-7.80 (1H, m), 8.60 (1H, s)

### 6-(7-tert-Butoxycarbonyl-6,8-dihydro-5H-pyrido[3,4-d]pyrimidin-2-yl)-1-(3-chlorophenyl)-7-oxo-4,5-dihydropyrazolo[3,4-c]pyridine-3-carboxylic acid

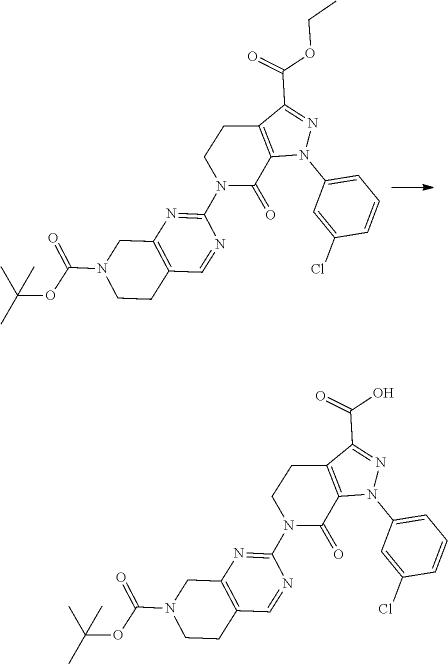 [SMILES: CCOC(=O)c1nn(-c2cccc(Cl)c2)c2c1CCN(c1ncc3c(n1)CN(C(=O)OC(C)(C)C)CC3)C2=O ; CC(C)(C)OC(=O)N1CCc2cnc(N3CCc4c(C(=O)O)nn(-c5cccc(Cl)c5)c4C3=O)nc2C1]

To a solution of tert-butyl 2-[1-(3-chlorophenyl)-3-ethoxycarbonyl-7-oxo-4,5-dihydropyrazolo[3,4-c]pyridin-6-yl]-6,8-dihydro-5H-pyrido[3,4-d]pyrimidine-7-carboxylate (95 mg, 0.17 mmol) in a mixture of THF (2 mL) and water (1 mL) was added LiOH·H₂O (18 mg, 0.43 mmol) and the mixture was stirred at rt for 72 h. The THF was removed and the remaining aqueous solution was acidified to pH=2-3 using 1M HCl (aq). The acidified aqueous phase was extracted with EtOAc (3×40 mL) and the combined organic extracts were dried (Na₂SO₄) and evaporated to afford the product (84 mg, 93% yield).

[M+H]⁺=543.1, 545.1

### tert-Butyl 2-[1-(3-chlorophenyl)-3-(cyclopropylmethylcarbamoyl)-7-oxo-4,5-dihydropyrazolo[3,4-c]pyridin-6-yl]-6,8-dihydro-5H-pyrido[3,4-d]pyrimidine-7-carboxylate

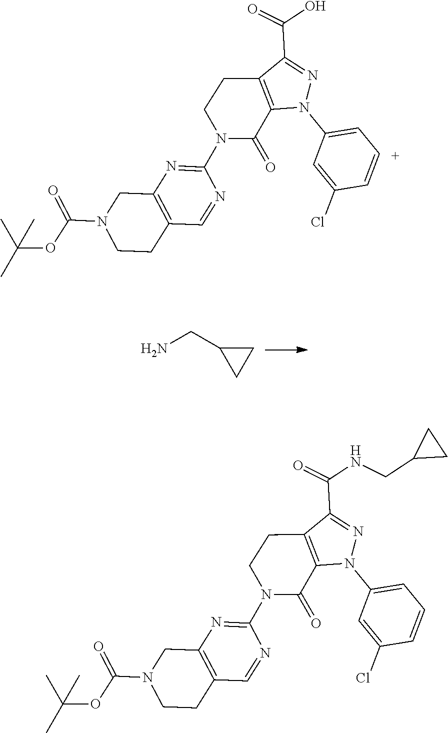 [SMILES: CC(C)(C)OC(=O)N1CCc2cnc(N3CCc4c(C(=O)O)nn(-c5cccc(Cl)c5)c4C3=O)nc2C1 ; NCC1CC1 ; CC(C)(C)OC(=O)N1CCc2cnc(N3CCc4c(C(=O)NCC5CC5)nn(-c5cccc(Cl)c5)c4C3=O)nc2C1]

To a suspension of 6-(7-tert-butoxycarbonyl-6,8-dihydro-5H-pyrido[3,4-d]pyrimidin-2-yl)-1-(3-chlorophenyl)-7-oxo-4,5-dihydropyrazolo[3,4-c]pyridine-3-carboxylic acid (80 mg, 0.15 mmol) in dry MeCN (2 mL), under N₂ (g), were added aminomethylcyclopropane (19 μL, 0.21 mmol), 1-methylimidazole (43 μL, 0.53 mmol) and TCFH (56 mg, 0.20 mmol). The mixture was stirred at rt for 20 h. This solution was diluted with EtOAc (75 mL), washed with sat. NaHCO₃ (20 mL) and brine (2×20 mL), dried (Na₂SO₄) and evaporated. The residue was purified by flash chromatography (silica, 5-100% EtOAc in iso-hexane) to afford the product (54 mg, 61% yield).

[M+H]⁺=578.4, 580.2

¹H NMR (DMSO-d6, 400 MHZ) δ 0.21-0.27 (2H, m), 0.38-0.45 (2H, m), 1.02-1.07 (1H, m), 1.42 (9H, s), 2.79 (2H, t, J=5.6 Hz), 3.11 (2H, t, J=6.4 Hz), 3.19 (2H, t, J=6.4 Hz), 3.61 (2H, s br), 4.22 (2H, t, J=6.4 Hz), 4.48 (2H, s), 7.50-7.60 (2H, m), 7.63 (1H, dt, J=7.3, 1.9 Hz), 7.80 (1H, t, J=2.0 Hz), 8.54 (1H, t, J=6.1 Hz), 8.60 (1H, s)

### 1-(3-Chlorophenyl)-N-(cyclopropylmethyl)-7-oxo-6-(5,6,7,8-tetrahydropyrido[3,4-d]pyrimidin-2-yl)-4,5-dihydropyrazolo[3,4-c]pyridine-3-carboxamide

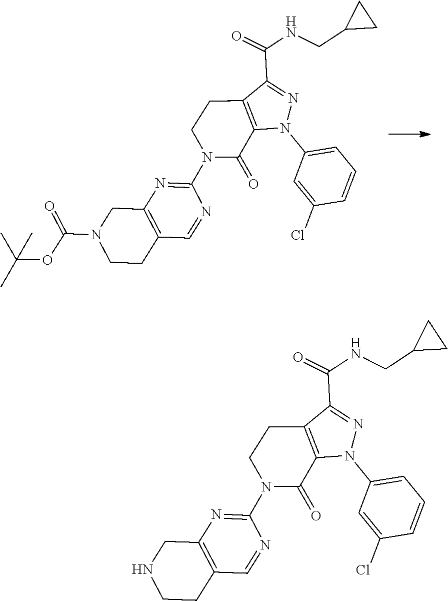 [SMILES: CC(C)(C)OC(=O)N1CCc2cnc(N3CCc4c(C(=O)NCC5CC5)nn(-c5cccc(Cl)c5)c4C3=O)nc2C1 ; O=C(NCC1CC1)c1nn(-c2cccc(Cl)c2)c2c1CCN(c1ncc3c(n1)CNCC3)C2=O]

To a solution of tert-butyl 2-[1-(3-chlorophenyl)-3-(cyclopropylmethylcarbamoyl)-7-oxo-4,5-dihydropyrazolo[3,4-c]pyridin-6-yl]-6,8-dihydro-5H-pyrido[3,4-d]pyrimidine-7-carboxylate (50 mg, 0.087 mmol) in 1,4-dioxane (0.5 mL), was added 4M HCl in 1,4-dioxane (0.50 mL) and the mixture was stirred at rt for 20 h. The solvent was evaporated and the residue was purified by ion-exchange SCX-2 column, washing with MeOH (10 mL) and eluting with 3M NH₃ in MeOH (10 mL) to afford the product (34 mg, 81%).

[M+H]⁺=478.1, 480.0

¹H NMR (DMSO-d6, 400 MHZ) δ 0.21-0.26 (2H, m), 0.38-0.45 (2H, m), 1.00-1.10 (1H, m), 2.66-2.71 (2H, m), 2.94 (2H, t, J=5.7 Hz), 3.11 (2H, t, J=6.5 Hz), 3.18 (2H, t, J=6.5 Hz), 3.78 (2H, s), 4.18 (2H, t, J=6.5 Hz), 7.49-7.58 (2H, m), 7.62 (1H, dt, J=7.3, 1.8 Hz), 7.79 (1H, td, J=2.0, 0.5 Hz), 8.49 (1H, s), 8.52 (1H, t, J=6.1 Hz)

### 1-(3-Chlorophenyl)-N-(cyclopropylmethyl)-6-(7-ethyl-5,6,7,8-tetrahydropyrido[3,4-d]pyrimidin-2-yl)-7

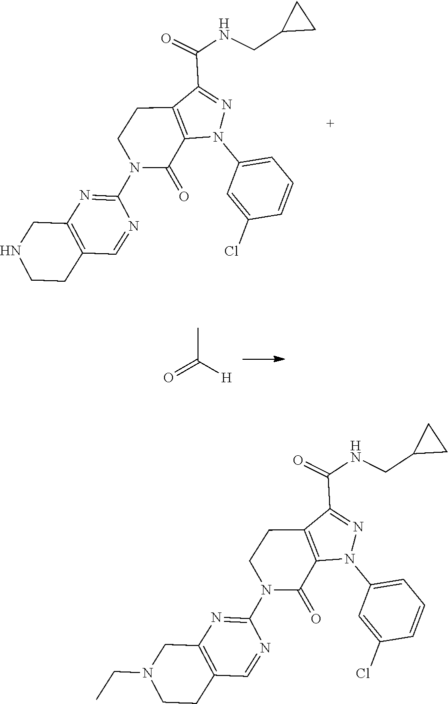

To a solution of 1-(3-chlorophenyl)-N-(cyclopropylmethyl)-7-oxo-6-(5,6,7,8-tetrahydropyrido[3,4-d] pyrimidin-2-yl)-4,5-dihydropyrazolo[3,4-c]pyridine-3-carboxamide (30 mg, 0.063 mmol) in a mixture of DCM (1 mL) and MeOH (0.2 mL) were added TEA (26 μL, 0.19 mmol) and acetaldehyde (50% in EtOH)(21 μL, 0.19 mmol) and the mixture was stirred at rt for 20 h. STAB (20 mg, 0.094 mmol) was added portion-wise and the mixture was stirred for a further 4 h. The mixture was diluted with DCM (50 mL), washed with sat. NaHCO₃(aq)(15 mL) and brine (15 mL), dried over (Na₂SO₄), purified by flash chromatography (silica, 0-10% MeOH in DCM) and further purified by ion-exchange SCX-2 column, washing with MeOH (10 mL) and eluting with 3M NH₃ in MeOH (10 mL) to afford the product (21 mg, 63% yield).

[M+H]⁺=506.1, 508.1

¹H NMR (DMSO-d6, 400 MHZ) δ 0.21-0.27 (2H, m), 0.38-0.45 (2H, m), 1.00-1.06 (1H, m), 1.08 (3H, t, J=7.1 Hz), 2.54 (2H, q, J=7.3 Hz), 2.70 (2H, t, J=5.7 Hz), 2.80 (2H, t, J=5.8 Hz), 3.11 (2H, t, J=6.5 Hz), 3.18 (2H, t, J=6.4 Hz), 3.53 (2H, s), 4.19 (2H, t, J=6.5 Hz), 7.49-7.58 (2H, m), 7.62 (1H, dt, J=7.3, 1.8 Hz), 7.79 (1H, t, J=2.0 Hz), 8.52 (1H, t, J=6.1 Hz), 8.54 (1H, s)

### Example Number 1066

### 1-(3-Chlorophenyl)-3-(methoxymethyl)-6-(2-methyl-1,2,3,4-tetrahydroisoquinolin-7-yl)-1,4,5,6-tetrahydro-7H-pyrazolo[3,4-c]pyridin-7-one

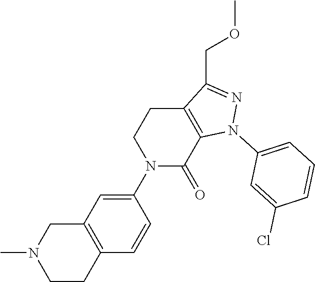 [SMILES: COCc1nn(-c2cccc(Cl)c2)c2c1CCN(c1ccc3c(c1)CN(C)CC3)C2=O]

### tert-Butyl 7-[1-(3-chlorophenyl)-3-(hydroxymethyl)-7-oxo-4,5-dihydropyrazolo[3,4-c]pyridin-6-yl]-3,4-dihydro-1H-isoquinoline-2-carboxylate

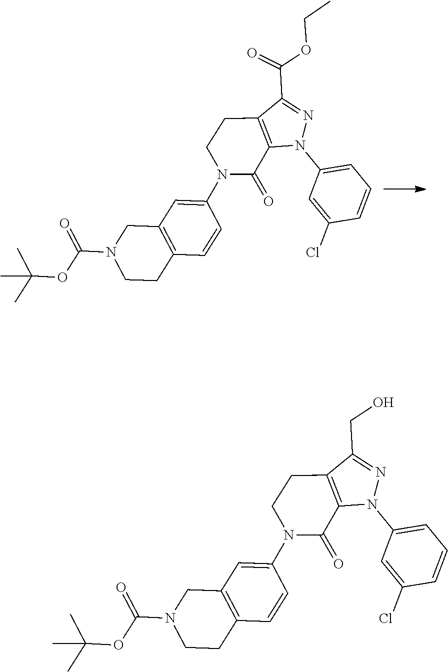 [SMILES: CCOC(=O)c1nn(-c2cccc(Cl)c2)c2c1CCN(c1ccc3c(c1)CN(C(=O)OC(C)(C)C)CC3)C2=O ; CC(C)(C)OC(=O)N1CCc2ccc(N3CCc4c(CO)nn(-c5cccc(Cl)c5)c4C3=O)cc2C1]

tert-Butyl 7-[1-(3-chlorophenyl)-3-ethoxycarbonyl-7-oxo-4,5-dihydropyrazolo[3,4-c]pyridin-6-yl]-3,4-dihydro-1H-isoquinoline-2-carboxylate (500 mg, 0.91 mmol) was dissolved in anhydrous toluene (50 mL). The solution was cooled to 0° C. and diisobutylaluminium hydride (1M solution in toluene)(1.9 mL, 1.9 mmol) was slowly added. The mixture was stirred at 0° C. to rt for 2 h. MeOH (2 mL) was slowly added.
After a further 5 min a saturated solution of sodium potassium tartrate (Rochelles salt)(20 mL) was added and stirring continued at rt for 30 min. The solid formed was filtered off through Celite® and the residue washed with EtOAc (100 mL). The combined filtrates were washed with water (1×30 mL), brine (1×30 mL), dried (Na₂SO₄) and evaporated. The residue was purified by flash chromatography (silica, 6% MeOH in CHCl₃) to afford the product (270 mg, 58% yield).

[M−Boc]⁺=409.2

### tert-Butyl 7-[1-(3-chlorophenyl)-3-(methoxymethyl)-7-oxo-4,5-dihydropyrazolo[3,4-c]pyridin-6-yl]-3,4-dihydro-1H-isoquinoline-2-carboxylate

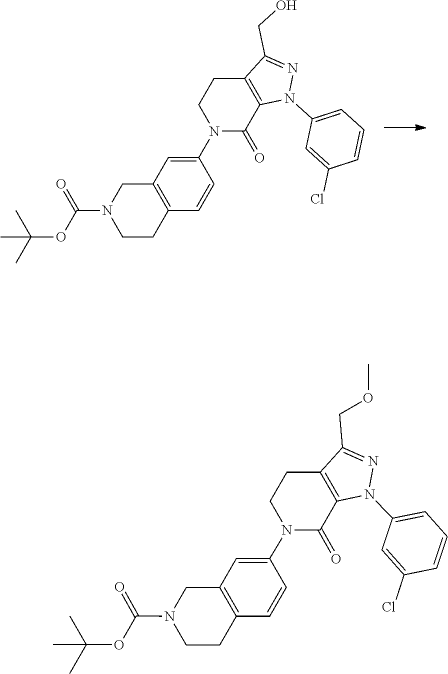 [SMILES: CC(C)(C)OC(=O)N1CCc2ccc(N3CCc4c(CO)nn(-c5cccc(Cl)c5)c4C3=O)cc2C1 ; COCc1nn(-c2cccc(Cl)c2)c2c1CCN(c1ccc3c(c1)CN(C(=O)OC(C)(C)C)CC3)C2=O]

tert-Butyl 7-[1-(3-chlorophenyl)-3-(hydroxymethyl)-7-oxo-4,5-dihydropyrazolo[3,4-c]pyridin-6-yl]-3,4-dihydro-1H-isoquinoline-2-carboxylate (260 mg, 0.51 mmol) was dissolved in THF (30 mL) at 0° C. NaH (60% oil dispersion)(25 mg, 0.61 mmol) was added and the suspension stirred at 0° C. for 15 min and at rt for a further 30 min. The reaction was cooled to 0° C. and Mel (35 μL, 0.56 mmol) was added and the mixture allowed to warm to rt over 3 h. The reaction was quenched with water (10 mL) and extracted with EtOAc (2×30 mL). The combined organic extracts were dried (Na₂SO₄), filtered and evaporated. The residue was purified by flash chromatography (Silica, 1% MeOH in CHCl₃) to afford the product (220 mg, 82% yield).

[M+Na]⁺=545.2

### 1-(3-Chlorophenyl)-3-(methoxymethyl)-6-(2-methyl-1,2,3,4-tetrahydroisoquinolin-7-yl)-1,4,5,6-tetrahydro-7H-pyrazolo[3,4-c]pyridin-7-one

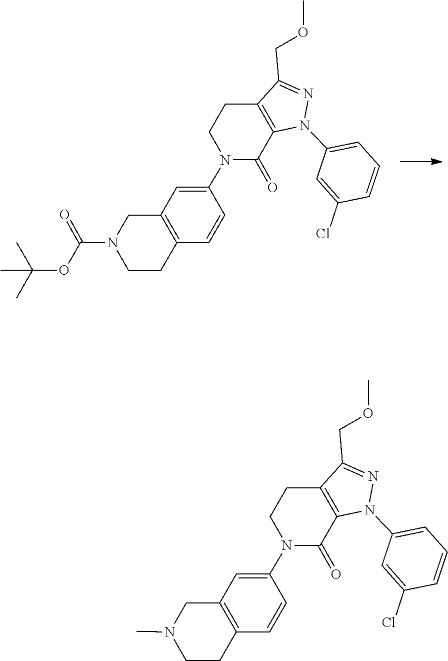 [SMILES: COCc1nn(-c2cccc(Cl)c2)c2c1CCN(c1ccc3c(c1)CN(C(=O)OC(C)(C)C)CC3)C2=O ; COCc1nn(-c2cccc(Cl)c2)c2c1CCN(c1ccc3c(c1)CN(C)CC3)C2=O]

A solution of tert-butyl 7-[1-(3-chlorophenyl)-3-(methoxymethyl)-7-oxo-4,5-dihydropyrazolo[3,4-c]pyridin-6-yl]-3,4-dihydro-1H-isoquinoline-2-carboxylate (72 mg, 0.14 mmol) in formic acid (52 μL, 1.38 mmol) was stirred at 50° C. for 30 min before formaldehyde (37% in water)(13 μL, 0.35 mmol) was added and the mixture heated to 90° C. for 2 h. The mixture was allowed to cool to rt and evaporated. The residue was purified by automated prep HPLC (mass directed 2-60% MeCN in water with an acidic mobile phase of 2.5% formic acid in water) to afford the product (22 mg, 36% yield).

[M+H]⁺=437.1

¹H NMR (DMSO-d6, 400 MHZ) δ 2.33 (3H, s), 2.58 (2H, t, J=5.9 Hz), 2.80 (2H, s), 2.96 (2H, s), 3.32 (3H, s), 3.45 (2H, s), 4.01 (2H, t, J=6.6 Hz), 4.51 (2H, s), 7.04 (1H, d, J=1.8 Hz), 7.11 (2H, d, J=1.8 Hz), 7.46-7.48 (2H, m), 7.51-7.56 (1H, m), 7.66 (1H, dt, J=2.5, 1.0 Hz)

### Example Number 1072

### 1-(3-Chlorophenyl)-N-(cyclopropylmethyl)-7-oxo-6-(3-(pyrrolidin-1-ylmethyl)phenyl)-4,5,6,7-tetrahydro-1H-pyrazolo[3,4-c]pyridine-3-carboxamide

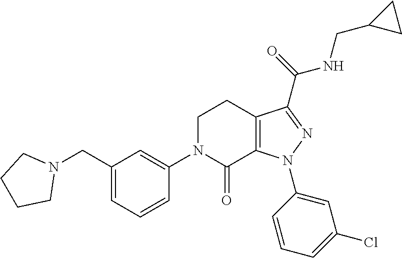 [SMILES: O=C(NCC1CC1)c1nn(-c2cccc(Cl)c2)c2c1CCN(c1cccc(CN3CCCC3)c1)C2=O]

### tert-Butyl-[(3-iodophenyl) methoxy]-dimethyl-silane

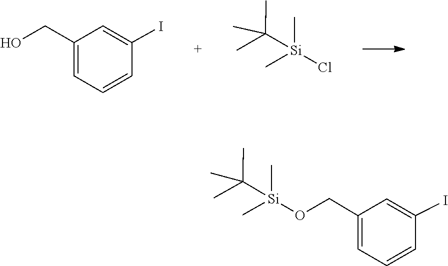

tert-Butyldimethylchlorosilane (773 mg, 5.13 mmol) was added to a solution of (3-iodophenyl) methanol (0.54 mL, 4.27 mmol) and imidazole (378 mg, 5.55 mmol) in DCM (40 mL) at rt. The mixture was stirred at rt for 20 h. Water (40 mL) was added, the organic phase was separated and the aqueous layer was extracted into DCM (2×10 mL). The combined organic extracts were washed with brine (40 mL), dried (Na₂SO₄) and evaporated. The residue was purified by chromatography (silica, 0-50% EtOAc in iso-hexane) to afford the product (868 mg, 58% yield).

¹H NMR (CDCl₃, 400 MHZ) δ 0.10 (6H, s), 0.94 (9H, s), 4.66-4.71 (2H, m), 7.06 (1H, t, J=7.7 Hz), 7.24-7.32 (1H, m), 7.53-7.61 (1H, m), 7.64-7.70 (1H, m)

### Ethyl 6-[3-[[tert-butyl(dimethyl) silyl]oxymethyl]phenyl]-1-(3-chlorophenyl)-7-oxo-4,5-dihydropyrazolo[3,4-c]pyridine-3-carboxylate

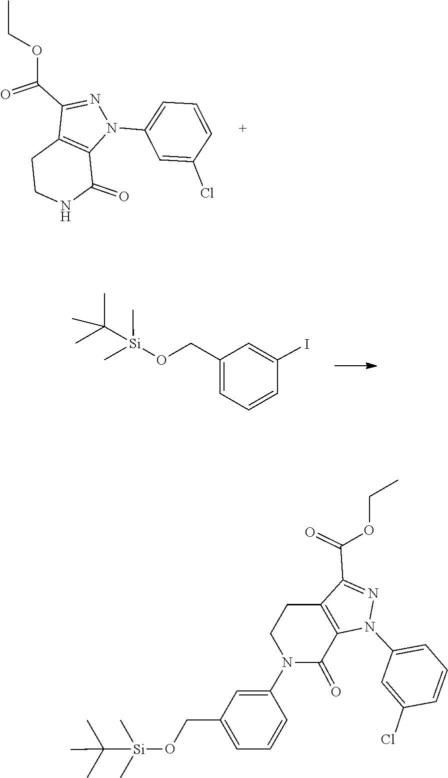 [SMILES: CCOC(=O)c1nn(-c2cccc(Cl)c2)c2c1CCNC2=O ; CC(C)(C)[Si](C)(C)OCc1cccc(I)c1 ; CCOC(=O)c1nn(-c2cccc(Cl)c2)c2c1CCN(c1cccc(CO[Si](C)(C)C(C)(C)C)c1)C2=O]

Cu(I)I (26 mg, 0.14 mmol) was added to a suspension of ethyl 1-(3-chlorophenyl)-7-oxo-5,6-dihydro-4H-pyrazolo[3,4-c]pyridine-3-carboxylate (220 mg, 0.69 mmol), tert-butyl-[(3-iodophenyl) methoxy]-dimethyl-silane (288 mg, 0.83 mmol), (1S,2S)—N,N′-dimethyl-1,2-cyclohexane-diamine (39 mg, 0.28 mmol) and K₃PO₄ (365 mg, 1.72 mmol) in anhydrous 1,4-dioxane (4.5 mL) at rt. The reaction vessel was sealed and heated with stirring at 100° C. for 20 h. The mixture was allowed to cool to rt, filtered through Celite® and the residue was washed with EtOAc (6×5 mL). The combined filtrates were washed with water (25 mL), brine (25 mL), dried (Na₂SO₄) and evaporated. The residue was purified by flash chromatography (silica, 0-60% EtOAc in iso-hexane) to afford the product (386 mg, 35% yield).

[M+H]⁺=540.2, 542.2

### 6-[3-[[tert-Butyl (dimethyl) silyl]oxymethyl]phenyl]-1-(3-chlorophenyl)-7-oxo-4,5-dihydropyrazolo[3,4-c]pyridine-3-carboxylic acid

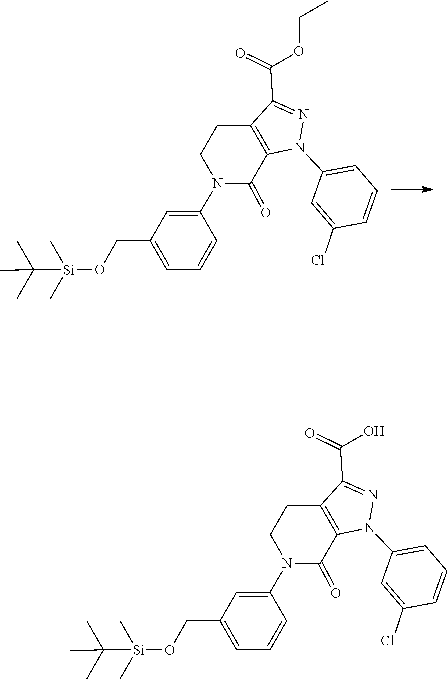 [SMILES: CCOC(=O)c1nn(-c2cccc(Cl)c2)c2c1CCN(c1cccc(CO[Si](C)(C)C(C)(C)C)c1)C2=O ; CC(C)(C)[Si](C)(C)OCc1cccc(N2CCc3c(C(=O)O)nn(-c4cccc(Cl)c4)c3C2=O)c1]

2M NaOH (aq)(0.54 mL, 1.1 mmol) was added to a solution of ethyl 6-[3-[[tert-butyl (dimethyl) silyl]oxymethyl]phenyl]-1-(3-chlorophenyl)-7-oxo-4,5-dihydropyrazolo[3,4-c]pyridine-3-carboxylate (386 mg, 0.715 mmol) in THF (3 mL) and MeOH (3 mL). The mixture was stirred at rt for 2 h. The solvent was evaporated, the residue was taken up in 5% citric acid (aq)(10 mL) and extracted into EtOAc (3×10 mL). The combined organic extracts were washed with brine (20 mL), dried (Na₂SO₄) and evaporated to afford the product (371 mg, 97% yield).

[M+H]⁺=512.1, 514.1

¹H NMR (DMSO-d6, 400 MHZ) δ 0.08 (6H, s), 0.90 (9H, s), 3.19 (2H, t, J=6.6 Hz), 4.07 (2H, t, J=6.6 Hz), 4.71 (2H, s), 7.20-7.34 (3H, m), 7.37 (1H, t, J=7.8 Hz), 7.46-7.62 (3H, m), 7.71-7.78 (1H, m), 13.34 (1H, s)

### 6-[3-[[tert-Butyl (dimethyl) silyl]oxymethyl]phenyl]-1-(3-chlorophenyl)-N-(cyclopropylmethyl)-7-oxo-4,5-dihydropyrazolo[3,4-c]pyridine-3-carboxamide

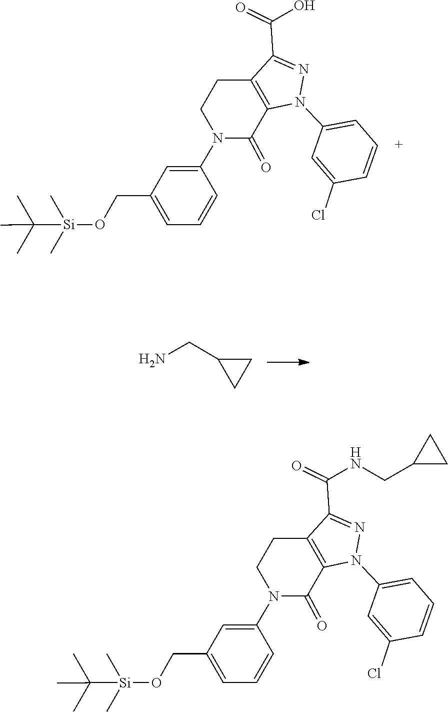 [SMILES: CC(C)(C)[Si](C)(C)OCc1cccc(N2CCc3c(C(=O)O)nn(-c4cccc(Cl)c4)c3C2=O)c1 ; NCC1CC1 ; CC(C)(C)[Si](C)(C)OCc1cccc(N2CCc3c(C(=O)NCC4CC4)nn(-c4cccc(Cl)c4)c3C2=O)c1]

TCFH (305 mg, 1.1 mmol) was added to a suspension of 6-[3-[[tert-butyl(dimethyl) silyl]oxymethyl]phenyl]-1-(3-chlorophenyl)-7-oxo-4,5-dihydropyrazolo[3,4-c]pyridine-3-carboxylic acid (371 mg, 0.73 mmol), aminomethylcyclopropane (75 μL, 0.87 mmol) and 1-methylimidazole (260 μL, 3.26 mmol) in anhydrous MeCN (4 mL). The mixture was stirred at rt for 20 h and the solvent evaporated. The residue was taken up in water (10 mL) and extracted with EtOAc (3×10 mL). The combined organic extracts were washed with sat. NaHCO₃(aq) and brine, dried (Na₂SO₄) and evaporated. The residue was purified by flash column chromatography (silica, 5-100% EtOAc in iso-hexane) to afford the product (360 mg, 81% yield).

[M+H]⁺=565.3, 567.3

¹H NMR (CDCl₃, 400 MHZ) δ 0.09 (6H, s), 0.25-0.33 (2H, m), 0.51-0.61 (2H, m), 0.93 (9H, s), 0.99-1.14 (1H, m), 3.28-3.35 (2H, m), 3.32-3.43 (2H, m), 4.07-4.13 (2H, m), 4.72 (2H, d, J=0.9 Hz), 7.07 (1H, t, J=5.7 Hz), 7.18-7.25 (3H, m), 7.29-7.42 (3H, m), 7.44-7.53 (1H, m), 7.59-7.65 (1H, m)

### N-Benzyl-1-(3-chlorophenyl)-6-[3-(hydroxymethyl)phenyl]-7-oxo-4,5-dihydropyrazolo[3,4-c]pyridine-3-carboxamide

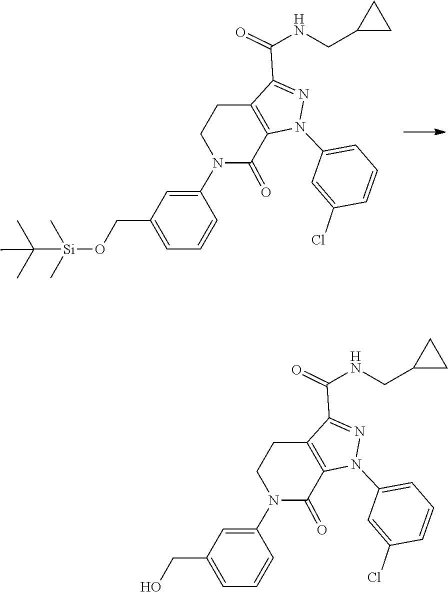 [SMILES: CC(C)(C)[Si](C)(C)OCc1cccc(N2CCc3c(C(=O)NCC4CC4)nn(-c4cccc(Cl)c4)c3C2=O)c1 ; O=C(NCC1CC1)c1nn(-c2cccc(Cl)c2)c2c1CCN(c1cccc(CO)c1)C2=O]

TBAF (1M in THF, 950 μL, 0.96 mmol) was added to a solution of N-benzyl-6-[3-[[tert-butyl (dimethyl) silyl]oxymethyl]phenyl]-1-(3-chlorophenyl)-7-oxo-4,5-dihydropyrazolo[3,4-c]pyridine-3-carboxamide (360 mg, 0.64 mmol) in THF (5 mL) at 0° C. The mixture was stirred at 0° C. for 2 h and the solvent evaporated. The residue was taken up in water (10 mL) and extracted with EtOAc (3×10 mL). The combined organic extracts were washed with brine, dried (Na₂SO₄) and evaporated. The residue was purified by flash chromatography (silica, 5-100% EtOAc in iso-hexane) to afford the product (216 mg, 73% yield).

[M+H]⁺=451.2, 453.2

¹H NMR (CDCl₃, 400 MHZ) δ 0.25-0.33 (2H, m), 0.51-0.62 (2H, m), 1.00-1.14 (1H, m), 1.73 (1H, t, J=6.0 Hz), 3.32 (2H, dd, J=7.1, 5.7 Hz), 3.40 (2H, dd, J=7.1, 6.4 Hz), 4.07-4.18 (2H, m), 4.70 (2H, d, J=5.8 Hz), 7.07 (1H, t, J=5.7 Hz), 7.20-7.28 (1H, m), 7.31-7.43 (4H, m), 7.46-7.53 (1H, m), 7.61 (1H, td, J=1.9, 0.5 Hz)

### 1-(3-Chlorophenyl)-N-(cyclopropylmethyl)-6-(3-formylphenyl)-7-oxo-4,5-dihydropyrazolo[3,4-c]pyridine-3-carboxamide

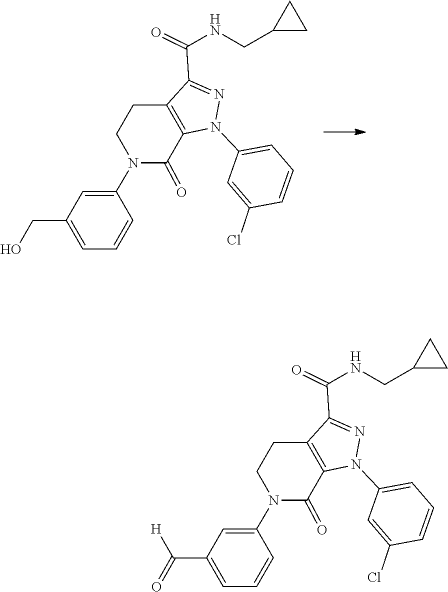 [SMILES: O=C(NCC1CC1)c1nn(-c2cccc(Cl)c2)c2c1CCN(c1cccc(CO)c1)C2=O ; O=Cc1cccc(N2CCc3c(C(=O)NCC4CC4)nn(-c4cccc(Cl)c4)c3C2=O)c1]

Activated MnO₂ (416 mg, 4.79 mmol) was added to a solution of 1-(3-chlorophenyl)-N-(cyclopropylmethyl)-6-[3-(hydroxymethyl)phenyl]-7-oxo-4,5-dihydropyrazolo[3,4-c]pyridine-3-carboxamide (216 mg, 0.48 mmol) in THF (5 mL). The mixture was heated to 40° C. with stirring for 20 h. The mixture was allowed to cool to rt, filtered through Celite® and the residue washed with THF (5×10 mL). The combined filtrates were evaporated to afford the product (229 mg, 100% yield).

[M+H]⁺=449.1, 451.1

¹H NMR (CDCl₃, 400 MHZ) δ 0.25-0.34 (2H, m), 0.53-0.62 (2H, m), 1.01-1.13 (1H, m), 3.28-3.36 (2H, m), 3.36-3.48 (2H, m), 4.18 (2H, t, J=6.7 Hz), 7.07 (1H, t, J=5.6 Hz), 7.37-7.64 (6H, m), 7.72-7.79 (1H, m), 7.84 (1H, t, J=1.9 Hz), 10.00 (1H, s)

### 1-(3-Chlorophenyl)-N-(cyclopropylmethyl)-7-oxo-6-(3-(pyrrolidin-1-ylmethyl)phenyl)-4,5,6,7-tetrahydro-1H-pyrazolo[3,4-c]pyridine-3-carboxamide

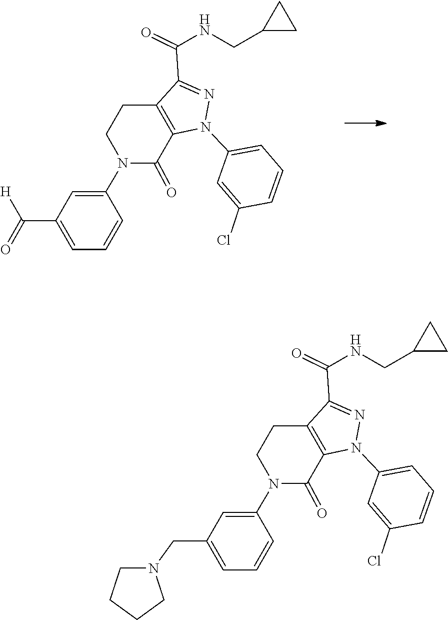 [SMILES: O=Cc1cccc(N2CCc3c(C(=O)NCC4CC4)nn(-c4cccc(Cl)c4)c3C2=O)c1 ; O=C(NCC1CC1)c1nn(-c2cccc(Cl)c2)c2c1CCN(c1cccc(CN3CCCC3)c1)C2=O]

AcOH (43 μL, 0.76 mmol) was added to a solution of 1-(3-chlorophenyl)-N-(cyclopropylmethyl)-6-(3-formylphenyl)-7-oxo-4,5-dihydropyrazolo[3,4-c]pyridine-3-carboxamide (110 mg, 0.25 mmol) and pyrrolidine (62 μL, 0.76 mmol) in a mixture of DCM (2 mL) and MeOH (0.4 mL) at rt. The mixture was stirred at rt for 2 h. STAB (80 mg, 0.38 mmol) was added and the resulting mixture was stirred at rt for 20 h. The mixture was quenched with sat. K₂CO₃ (aq)(10 mL) and extracted into DCM (3×5 mL). The combined organic extracts were washed with brine (10 mL), dried (Na₂SO₄) and evaporated. The residue was purified by flash chromatography (silica, 0-15% MeOH in DCM), further purified by SCX ion-exchange, loaded with MeOH (10 mL) and eluted with 2M NH₃ in MeOH (10 mL) and further purified with preparative HPLC (Mass-directed Prep LC, eluted with 5-95% MeCN in water, both containing 0.1% NH₃) to afford the product (9.8 mg, 8% yield).

[M+H]⁺=504.1, 506.1

¹H NMR (DMSO-d6, 400 MHz) δ 0.20-0.28 (2H, m), 0.37-0.46 (2H, m), 1.00-1.09 (1H, m), 1.65-1.73 (4H, m), 2.38-2.44 (4H, m), 3.12 (2H, t, J=6.5 Hz), 3.20 (2H, t, J=6.6 Hz), 3.56 (2H, s), 4.06 (2H, t, J=6.6 Hz), 7.15-7.26 (2H, m), 7.26-7.37 (2H, m), 7.46-7.57 (2H, m), 7.59-7.66 (1H, m), 7.76-7.81 (1H, m), 8.51 (1H, t, J=6.0 Hz)

### Example Number 1117

### 1-(3-Chlorophenyl)-N-(cyclopropylmethyl)-6-(6-ethyl-5,6,7,8-tetrahydro-2,6-naphthyridin-3-yl)-7-oxo-4,5,6,7-tetrahydro-1H-pyrazolo[3,4-c]pyridine-3-carboxamide

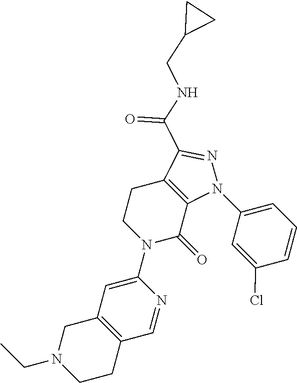 [SMILES: CCN1CCc2cnc(N3CCc4c(C(=O)NCC5CC5)nn(-c5cccc(Cl)c5)c4C3=O)cc2C1]

### tert-Butyl 7-chloro-3,4-dihydro-1H-2,6-naphthyridine-2-carboxylate

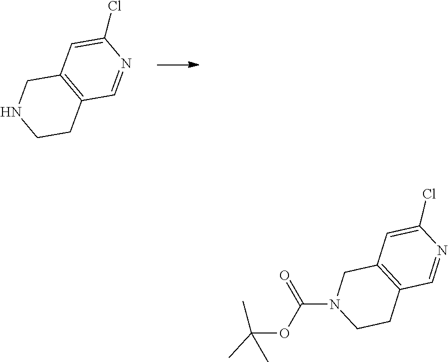 [SMILES: Clc1cc2c(cn1)CCNC2 ; CC(C)(C)OC(=O)N1CCc2cnc(Cl)cc2C1]

Boc₂O (346 mg, 1.58 mmol) was added to a solution of 7-chloro-1,2,3,4-tetrahydro-2,6-naphthyridine hydrochloride (1:1)(250 mg, 1.22 mmol) and TEA (0.42 mL, 3.05 mmol) in DCM (10 mL) at rt. The mixture was stirred at rt for 20 h. Water (20 mL) was added, the organic phase was separated, and the aqueous fraction was extracted with DCM (2×15 mL). The organic fractions were combined, washed with sat. brine solution (20 mL) and dried (Na₂SO₄). The resulting mixture was filtered and concentrated to give the product (315 mg, 96% yield) which was used in subsequent reactions without further purification.

[M+H]⁺=269.1/271.1

¹H NMR (DMSO-d6, 400 MHZ) δ 1.42 (10H, d, J=2.1 Hz), 2.76 (2H, t, J=5.9 Hz), 3.51-3.59 (2H, m), 4.54 (2H, s), 7.41 (1H, s), 8.22 (1H, d, J=2.0 Hz)

### tert-Butyl 7-[1-(3-chlorophenyl)-3-ethoxycarbonyl-7-oxo-4,5-dihydropyrazolo[3,4-c]pyridin-6-yl]-3,4-dihydro-1H-2,6-naphthyridine-2-carboxylate

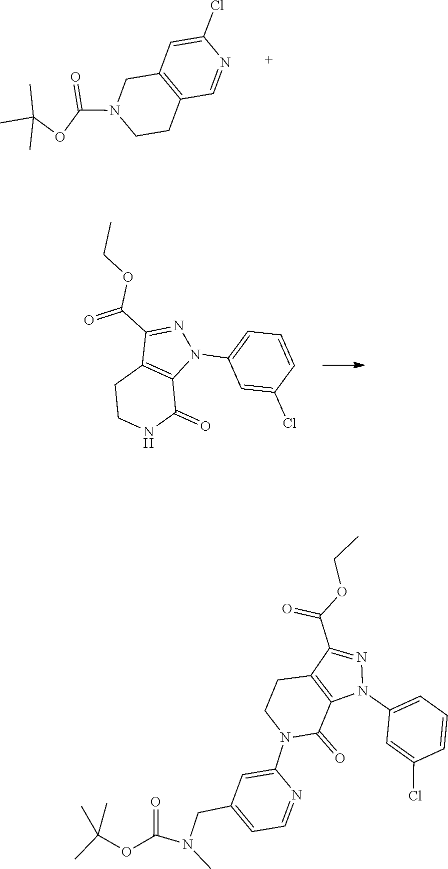

Pd₂(dba)₃ (83 mg, 0.0907 mmol) and XantPhos (131 mg, 0.227 mmol) were added to a N₂ (g) sparged suspension of ethyl 1-(3-chlorophenyl)-7-oxo-5,6-dihydro-4H-pyrazolo[3,4-c]pyridine-3-carboxylate (290 mg, 0.907 mmol), tert-butyl 7-chloro-3,4-dihydro-1H-2,6-naphthyridine-2-carboxylate (313 mg, 1.09 mmol) and Cs₂CO₃ (591 mg, 1.81 mmol) in anhydrous 1,4-dioxane (6.4 mL) at rt. The mixture was sparged with N₂ (g) for a further 5 min, then heated at 100° C. for 18 h. The mixture was allowed to cool to rt, and filtered through a pad of Celite®. The filtrate was washed with EtOAc (5×15 mL). The filtrates were combined and washed sequentially with water (30 ml) and brine (30 mL), dried (Na₂SO₄), filtered, concentrated, and purified by flash chromatography (silica, 0-100% EtOAc in iso-hexane) to yield the product (385 mg 75% yield).

[M+H]⁺=552.3/554.3

¹H NMR (DMSO-d6, 400 MHZ) δ 1.33 (3H, t, J=7.1 Hz), 1.41 (9H, s), 2.77 (2H, t, J=5.7 Hz), 3.16 (2H, t, J=6.5 Hz), 3.57 (2H, t, J=5.6 Hz), 4.25-4.44 (3H, m), 4.50 (2H, s), 7.48 (1H, s), 7.49-7.60 (2H, m), 7.60 (1H, tt, J=6.4, 1.9 Hz), 7.78 (1H, t, J=2.0 Hz), 8.28 (1H, s)

### 6-(6-tert-Butoxycarbonyl-7,8-dihydro-5H-2,6-naphthyridin-3-yl)-1-(3-chlorophenyl)-7-oxo-4,5-dihydropyrazolo[3,4-c]pyridine-3-carboxylic acid

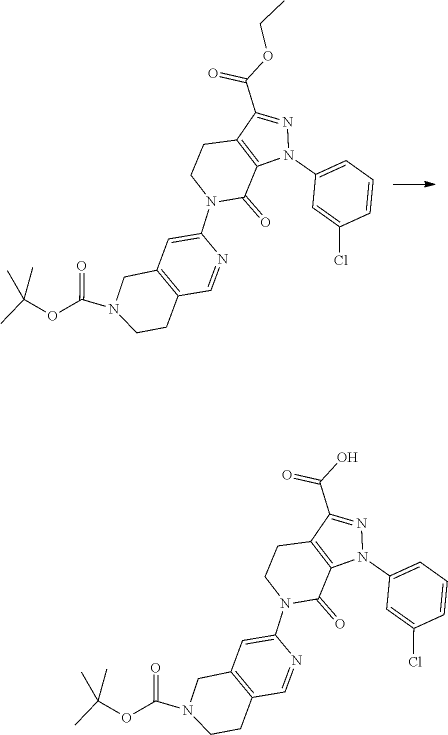 [SMILES: CCOC(=O)c1nn(-c2cccc(Cl)c2)c2c1CCN(c1cc3c(cn1)CCN(C(=O)OC(C)(C)C)C3)C2=O ; CC(C)(C)OC(=O)N1CCc2cnc(N3CCc4c(C(=O)O)nn(-c5cccc(Cl)c5)c4C3=O)cc2C1]

A solution of tert-butyl 7-[1-(3-chlorophenyl)-3-ethoxycarbonyl-7-oxo-4,5-dihydropyrazolo[3,4-c]pyridin-6-yl]-3,4-dihydro-1H-2,6-naphthyridine-2-carboxylate (385 mg, 0.697 mmol) in a mixture of THF (5 mL) and MeOH (5 mL) was treated with 2M NaOH (aq)(0.70 mL, 1.4 mmol) and stirred at rt for 18 h. The mixture was concentrated, acidified with 1M HCl to PH ˜ 3 and extracted with EtOAc (3×50 mL). The combined organics were washed with brine (30 mL), dried (Na₂SO₄), filtered and concentrated to afford the product (254 mg, 54% yield).

[M+H]⁺=524.1/526.1

### tert-Butyl 7-[1-(3-chlorophenyl)-3-(cyclopropylmethylcarbamoyl)-7-oxo-4,5-dihydropyrazolo[3,4-c]pyridin-6-yl]-3,4-dihydro-1H-2,6-naphthyridine-2-carboxylate

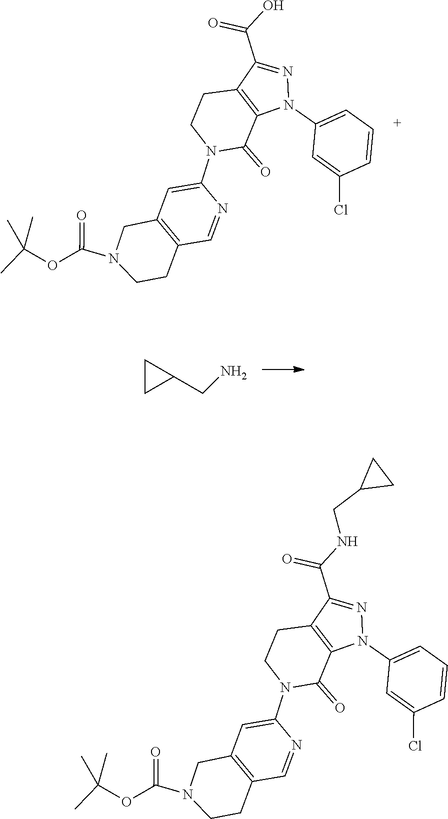 [SMILES: CC(C)(C)OC(=O)N1CCc2cnc(N3CCc4c(C(=O)O)nn(-c5cccc(Cl)c5)c4C3=O)cc2C1 ; NCC1CC1 ; CC(C)(C)OC(=O)N1CCc2cnc(N3CCc4c(C(=O)NCC5CC5)nn(-c5cccc(Cl)c5)c4C3=O)cc2C1]

TCFH (80 mg, 0.29 mmol) was added to a suspension of 6-(6-tert-butoxycarbonyl-7,8-dihydro-5H-2,6-naphthyridin-3-yl)-1-(3-chlorophenyl)-7-oxo-4,5-dihydropyrazolo[3,4-c]pyridine-3-carboxylic acid (100 mg, 0.191 mmol), aminomethylcyclopropane (0.020 mL, 0.23 mmol) and 1-methylimidazole (0.068 mL, 0.86 mmol) in anhydrous MeCN (2 mL) at rt. The mixture was stirred at rt for 20 h. The organics were evaporated, water (10 mL) was added and extracted with EtOAc (3×15 mL). The extracts were combined, washed with sat. NaHCO₃(aq)(10 mL) and sat. brine solution (10 mL), dried (Na₂SO₄), filtered, concentrated and purified by flash chromatography (silica, 0-100% EtOAc in iso-hexane) to yield the product (91 mg, 74% yield).

[M+H]⁺=577.3/579.4

¹H NMR (DMSO-d6, 400 MHZ) δ 0.18-0.32 (2H, m), 0.35-0.53 (2H, m), 0.99-1.11 (1H, m), 1.41 (9H, s), 2.77 (2H, t, J=5.8 Hz), 3.12 (2H, t, J=6.4 Hz), 3.16 (2H, t, J=6.5 Hz), 3.57 (2H, s), 4.29 (2H, t, J=6.4 Hz), 4.50 (2H, s), 7.45-7.60 (3H, m), 7.64 (1H, dt, J=6.9, 2.0 Hz), 7.81 (1H, t, J=1.6 Hz), 8.28 (1H, s), 8.53 (1H, t, J=6.0 Hz)

### 1-(3-Chlorophenyl)-N-(cyclopropylmethyl)-7-oxo-6-(5,6,7,8-tetrahydro-2,6-naphthyridin-3-yl)-4,5-dihydropyrazolo[3,4-c]pyridine-3-carboxamide hydrochloride

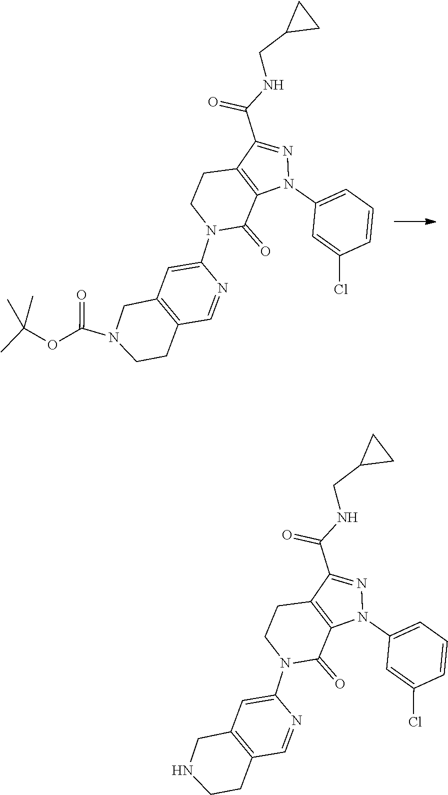 [SMILES: CC(C)(C)OC(=O)N1CCc2cnc(N3CCc4c(C(=O)NCC5CC5)nn(-c5cccc(Cl)c5)c4C3=O)cc2C1 ; O=C(NCC1CC1)c1nn(-c2cccc(Cl)c2)c2c1CCN(c1cc3c(cn1)CCNC3)C2=O]

HCl (4N in 1,4-dioxane)(0.83 mL, 3.3 mmol) was added to a stirring solution of tert-butyl 7-[1-(3-chlorophenyl)-3-(cyclopropylmethylcarbamoyl)-7-oxo-4,5-dihydropyrazolo[3,4-c]pyridin-6-yl]-3,4-dihydro-1H-2,6-naphthyridine-2-carboxylate (91 mg, 0.16 mmol) in 1,4-dioxane (0.8 mL) at rt. The mixture was stirred at rt for 18 h. Solvent was removed to afford the product (69 mg, 75% yield).

[M+H]⁺=477.3/479.3

¹H NMR (DMSO-d6, 400 MHZ) δ 0.15-0.26 (2H, m), 0.30-0.45 (2H, m), 0.94-1.05 (1H, m), 2.97 (2H, d, J=5.9 Hz), 3.08 (2H, t, J=6.5 Hz), 3.14 (2H, t, J=6.2 Hz), 3.35 (2H, d, J=6.0 Hz), 4.18-4.38 (4H, m), 7.45-7.54 (2H, m), 7.55 (1H, s), 7.61 (1H, dt, J=6.9, 2.0 Hz), 7.78 (1H, t, J=1.9 Hz), 8.32 (1H, s), 8.50 (1H, t, J=5.9 Hz), 9.25 (2H, br s)

### 1-(3-Chlorophenyl)-N-(cyclopropylmethyl)-6-(6-ethyl-5,6,7,8-tetrahydro-2,6-naphthyridin-3-yl)-7-oxo-4,5,6,7-tetrahydro-1H-pyrazolo[3,4-c]pyridine-3-carboxamide

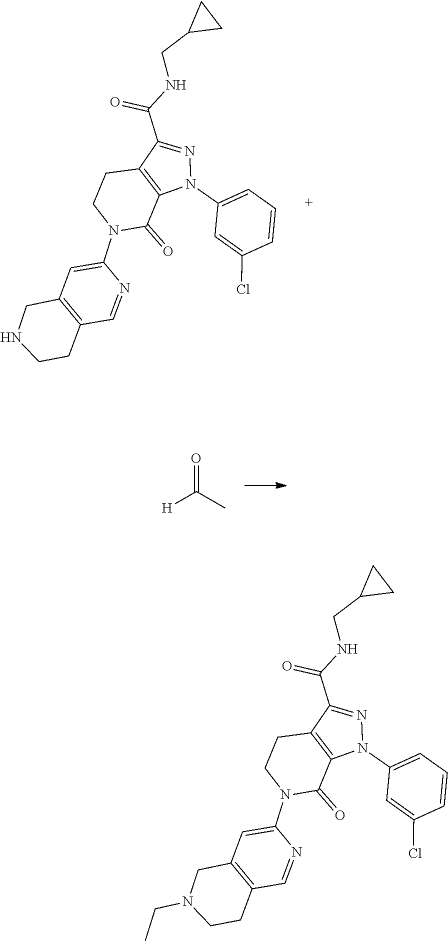

To a solution of 1-(3-chlorophenyl)-N-(cyclopropylmethyl)-7-oxo-6-(5,6,7,8-tetrahydro-2,6-naphthyridin-3-yl)-4,5-dihydropyrazolo[3,4-c]pyridine-3-carboxamide hydrochloride (33 mg, 0.064 mmol) in a mixture of DCM (0.8 mL) and MeOH (0.2 mL) were added TEA (0.027 mL, 0.19 mmol) and acetaldehyde (0.0072 mL, 0.13 mmol) and the mixture stirred at rt for 1 h. STAB (20 mg, 0.096 mmol) was added portion-wise and the mixture was stirred for 18 h. The mixture was diluted with DCM (20 mL) and washed with sat. NaHCO₃(aq)(10 mL) and brine (10 mL), dried (Na₂SO₄), filtered, evaporated, purified by flash chromatography (silica, 0-8% MeOH in DCM), further purified by loading onto an SCX-2 and washing with DCM (10 mL), eluting with 3M NH₃ in MeOH (15 mL), and lyophilised to provide the product (14 mg, 41% yield).

[M+H]⁺=505.3.1/507.3

¹H NMR (DMSO-d6, 400 MHZ) δ 0.15-0.28 (2H, m), 0.34-0.56 (2H, m), 0.98-1.15 (4H, m), 2.67 (2H, s), 2.80 (2H, s), 3.12 (2H, t, J=6.5 Hz), 3.16 (2H, t, J=6.5 Hz), 3.52 (2H, s), 4.29 (2H, t, J=6.5 Hz), 7.40 (1H, s), 7.47-7.59 (2H, m), 7.64 (1H, dt, J=7.0, 2.0 Hz), 7.81 (1H, d, J=2.2 Hz), 8.23 (1H, s), 8.51 (1H, t, J=6.1 Hz) 2H obscured by DMSO peak

### Example Number 1122

### 1-(3-Chlorophenyl)-N-(cyclopropylmethyl)-6-(5-fluoro-2-(2-hydroxyethyl)-1,2,3,4-tetrahydroisoquinolin-7-yl)-7-oxo-4,5,6,7-tetrahydro-1H-pyrazolo[3,4-c]pyridine-3-carboxamide

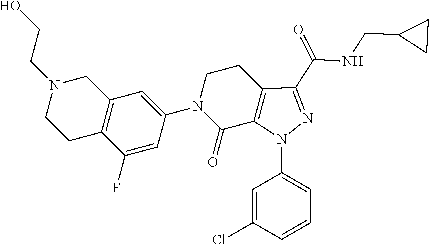 [SMILES: O=C(NCC1CC1)c1nn(-c2cccc(Cl)c2)c2c1CCN(c1cc(F)c3c(c1)CN(CCO)CC3)C2=O]

### tert-Butyl 7-[1-(3-chlorophenyl)-3-ethoxycarbonyl-7-oxo-4,5-dihydropyrazolo[3,4-c]pyridin-6-yl]-5-fluoro-3,4-dihydro-1H-isoquinoline-2-carboxylate

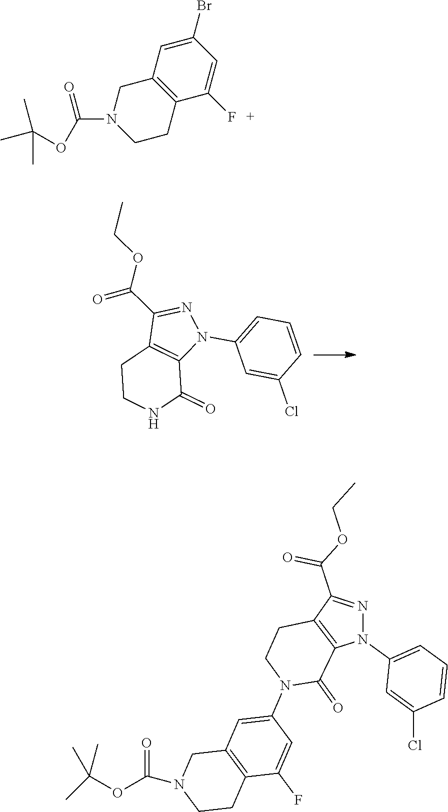

A solution of ethyl 1-(3-chlorophenyl)-7-oxo-5,6-dihydro-4H-pyrazolo[3,4-c]pyridine-3-carboxylate (220 mg, 0.688 mmol) and tert-butyl 7-bromo-5-fluoro-3,4-dihydro-1H-isoquinoline-2-carboxylate (250 mg, 0.757 mmol) in 1,4-dioxane (8 mL), was bubbled with N₂ (g) for 5 min. After this time, whilst maintaining the bubbling, were sequentially added K₃PO₄ (365 mg, 1.72 mmol), (1S,2S)—N,N′-dimethyl-1,2-cyclohexanediamine (39 mg, 0.275 mmol) and, finally, Cu(I)I (26 mg, 0.14 mmol). After 2 more min bubbling with N₂ (g), the system was closed and stirred at 100° C. for 18 h. The solution was filtered through Celite®, rinsing with EtOAc (50 mL), and the filtrate was washed with water and brine, dried (Na₂SO₄), filtered and evaporated. Flash chromatography (silica, 2-50% EtOAc in iso-hexane) afforded the product (185 mg, 47% yield).

[M−tBu+H]⁺=513.1/515.1

¹H NMR (DMSO-d6, 400 MHZ) δ 1.33 (3H, t, J=7.1 Hz), 1.42 (9H, s), 2.70 (2H, t, J=5.9 Hz), 3.19 (2H, t, J=6.6 Hz), 3.58 (2H, t, J=5.9 Hz), 4.07 (2H, t, J=6.6 Hz), 4.35 (2H, q, J=7.1 Hz), 4.52 (2H, s), 7.08 (1H, d, J=2.0 Hz), 7.12 (1H, d, J=10.9 Hz), 7.49-7.58 (2H, m), 7.58-7.64 (1H, m), 7.72-7.76 (1H, m)

### 6-(2-tert-Butoxycarbonyl-5-fluoro-3,4-dihydro-1H-isoquinolin-7-yl)-1-(3-chlorophenyl)-7-oxo-4,5-dihydropyrazolo[3,4-c]pyridine-3-carboxylic acid

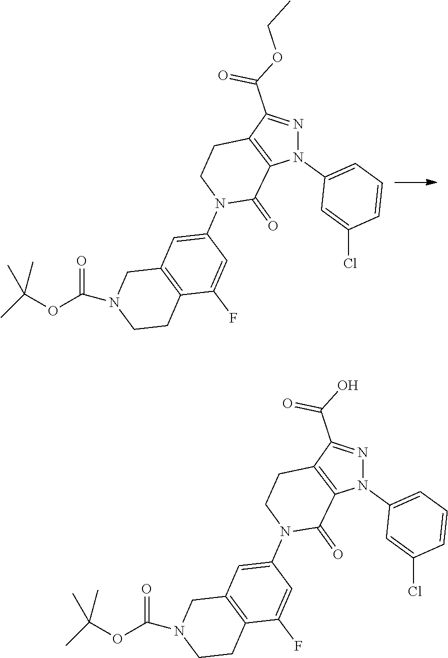 [SMILES: CCOC(=O)c1nn(-c2cccc(Cl)c2)c2c1CCN(c1cc(F)c3c(c1)CN(C(=O)OC(C)(C)C)CC3)C2=O ; CC(C)(C)OC(=O)N1CCc2c(F)cc(N3CCc4c(C(=O)O)nn(-c5cccc(Cl)c5)c4C3=O)cc2C1]

To a solution of tert-butyl 7-[1-(3-chlorophenyl)-3-ethoxycarbonyl-7-oxo-4,5-dihydropyrazolo[3,4-c]pyridin-6-yl]-5-fluoro-3,4-dihydro-1H-isoquinoline-2-carboxylate (180 mg, 0.316 mmol) in a mixture of THF (2 mL) and water (1 mL) was added LiOH·H₂O (33 mg, 0.79 mmol). The mixture was stirred at rt for 18 h. The THF was evaporated under reduced pressure and the remaining aqueous solution was acidified to pH=2-3 using 1M HCl (aq). The acidified aqueous phase was extracted with EtOAc and the combined organic phases were dried (Na₂SO₄), filtered and evaporated to provide the product (160 mg, 94% yield).

[M−tBu+H]⁺=502.1/504.2

¹H NMR (DMSO-d6, 400 MHZ) δ 1.42 (9H, s), 2.71 (2H, t, J=5.9 Hz), 3.18 (2H, t, J=6.6 Hz), 3.58 (2H, t, J=5.9 Hz), 4.06 (2H, t, J=6.6 Hz), 4.52 (2H, s), 7.08 (1H, d, J=2.0 Hz), 7.12 (1H, dd, J=10.8, 1.9 Hz), 7.48-7.57 (2H, m), 7.59 (1H, dt, J=7.0, 2.0 Hz), 7.72-7.75 (1H, m), 13.34 (1H, s br)

### tert-Butyl 7-[1-(3-chlorophenyl)-3-(cyclopropylmethylcarbamoyl)-7-oxo-4,5-dihydropyrazolo[3,4-c]pyridin-6-yl]-5-fluoro-3,4-dihydro-1H-isoquinoline-2-carboxylate

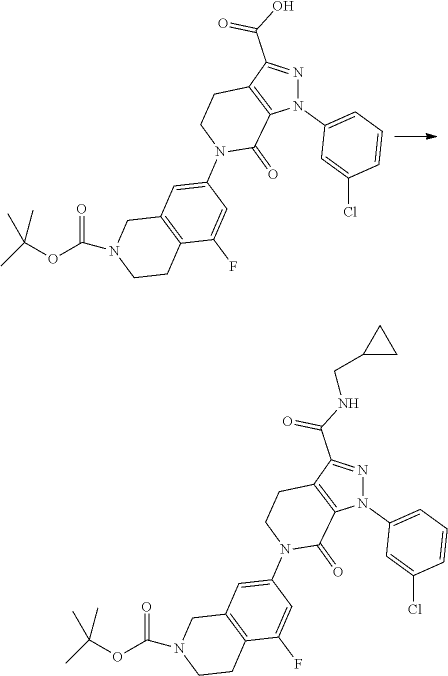 [SMILES: CC(C)(C)OC(=O)N1CCc2c(F)cc(N3CCc4c(C(=O)O)nn(-c5cccc(Cl)c5)c4C3=O)cc2C1 ; CC(C)(C)OC(=O)N1CCc2c(F)cc(N3CCc4c(C(=O)NCC5CC5)nn(-c5cccc(Cl)c5)c4C3=O)cc2C1]

To a suspension of 6-(2-tert-butoxycarbonyl-5-fluoro-3,4-dihydro-1H-isoquinolin-7-yl)-1-(3-chlorophenyl)-7-oxo-4,5-dihydropyrazolo[3,4-c]pyridine-3-carboxylic acid (160 mg, 0.296 mmol) in MeCN (3 mL) under N₂ (g) were added aminomethylcyclopropane (0.051 mL, 0.59 mmol), 1-methylimidazole (0.094 mL, 1.2 mmol) and TCFH (124 mg, 0.444 mmol), and the mixture was stirred for 72 h at rt. The mixture was diluted with EtOAc (50 mL) and washed with sat. NaHCO₃(aq) and brine, dried (Na₂SO₄), filtered and evaporated. Flash chromatography (silica, 2-45% EtOAc in iso-hexane) afforded the product (131 mg, 74% yield).

[M−tBu+H]⁺=538.1/540.1

¹H NMR (DMSO-d6, 400 MHZ) δ 0.21-0.27 (2H, m), 0.39-0.46 (2H, m), 1.00-1.09 (1H, m), 1.42 (9H, s), 2.71 (2H, t, J=5.9 Hz), 3.12 (2H, t, J=6.4 Hz), 3.20 (2H, t, J=6.6 Hz), 3.58 (2H, t, J=6.0 Hz), 4.05 (2H, t, J=6.7 Hz), 4.52 (2H, s), 7.08 (1H, d, J=2.0 Hz), 7.12 (1H, dd, J=10.8, 2.0 Hz), 7.45-7.60 (2H, m), 7.63 (1H, dt, J=6.4, 2.2 Hz), 7.76-7.80 (1H, m), 8.52 (1H, t, J=6.0 Hz)

### 1-(3-Chlorophenyl)-N-(cyclopropylmethyl)-6-(5-fluoro-1,2,3,4-tetrahydroisoquinolin-7-yl)-7-oxo-4,5-dihydropyrazolo[3,4-c]pyridine-3-carboxamide hydrochloride

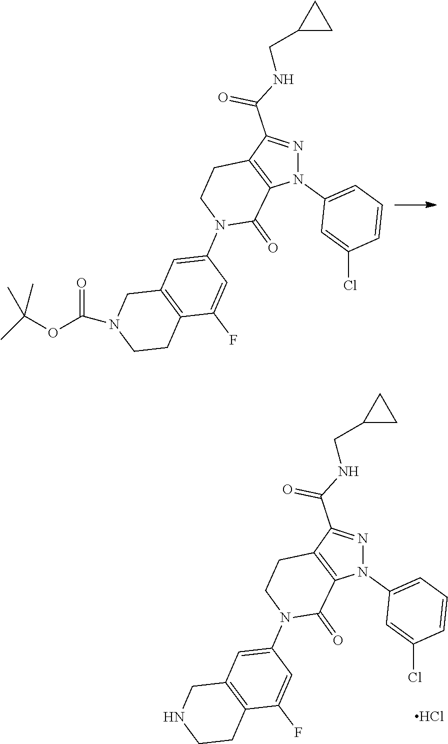 [SMILES: CC(C)(C)OC(=O)N1CCc2c(F)cc(N3CCc4c(C(=O)NCC5CC5)nn(-c5cccc(Cl)c5)c4C3=O)cc2C1 ; O=C(NCC1CC1)c1nn(-c2cccc(Cl)c2)c2c1CCN(c1cc(F)c3c(c1)CNCC3)C2=O ; Cl]

To a solution of tert-butyl 7-[1-(3-chlorophenyl)-3-(cyclopropylmethylcarbamoyl)-7-oxo-4,5-dihydropyrazolo[3,4-c]pyridin-6-yl]-5-fluoro-3,4-dihydro-1H-isoquinoline-2-carboxylate (130 mg, 0.219 mmol) in 1,4-dioxane (2 mL) was added HCl solution (4M in 1,4-dioxane)(1.5 mL, 6.0 mmol) and the mixture was stirred for 20 h at rt. The solvent was evaporated to provide the product (123 mg).

[M+H]⁺=494.2/496.1

¹H NMR (DMSO-d6, 400 MHZ) δ 0.21-0.27 (2H, m), 0.39-0.45 (2H, m), 0.99-1.11 (1H, m), 2.93 (2H, t, J=6.3 Hz), 3.12 (2H, t, J=6.4 Hz), 3.22 (2H, t, J=6.6 Hz), 3.40 (2H, t, J=7.2 Hz), 4.05 (2H, t, J=6.6 Hz), 4.28 (2H, s), 7.15 (1H, s), 7.25 (1H, d, J=10.7 Hz), 7.49-7.57 (2H, m), 7.63 (1H, dt, J=6.8, 2.1 Hz), 7.77-7.81 (1H, m), 8.53 (1H, t, J=6.0 Hz), 9.29 (2H, s br)

### 1-(3-Chlorophenyl)-N-(cyclopropylmethyl)-6-(5-fluoro-2-(2-hydroxyethyl)-1,2,3,4-tetrahydroisoquinolin-7-yl)-7-oxo-4,5,6,7-tetrahydro-1H-pyrazolo[3,4-c]pyridine-3-carboxamide

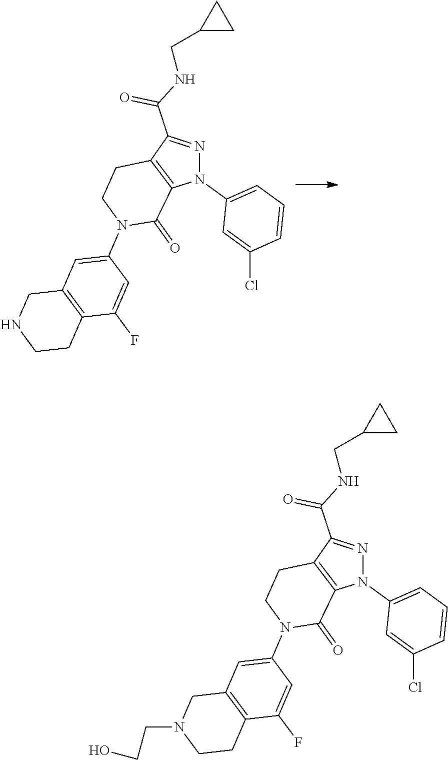 [SMILES: O=C(NCC1CC1)c1nn(-c2cccc(Cl)c2)c2c1CCN(c1cc(F)c3c(c1)CNCC3)C2=O ; O=C(NCC1CC1)c1nn(-c2cccc(Cl)c2)c2c1CCN(c1cc(F)c3c(c1)CN(CCO)CC3)C2=O]

To a solution of 1-(3-chlorophenyl)-N-(cyclopropylmethyl)-6-(5-fluoro-1,2,3,4-tetrahydroisoquinolin-7-yl)-7-oxo-4,5-dihydropyrazolo[3,4-c]pyridine-3-carboxamide hydrochloride (60 mg, 0.11 mmol) in DMF (1.5 mL), under N₂ (g), were added TEA (0.055 mL, 0.40 mmol) and 2-bromoethanol (0.024 mL, 0.34 mmol), and the mixture was stirred for 20 h at rt. The solution was diluted with EtOAc (50 mL) and washed with sat. NaHCO₃(aq) and brine, dried (Na₂SO₄), filtered and evaporated. The product was purified by flash chromatography (silica, 0-7% MeOH in DCM). The product was purified further by loading it onto an ion-exchange SCX-2 column, washing with MeOH and eluting with 3M NH₃ in MeOH (10 mL). The eluent was concentrated and lyophilised to provide the product (32 mg, 52% yield).

[M+H]⁺=538.3/540.3

¹H NMR (DMSO-d6, 400 MHZ) δ 0.24 (2H, dt, J=6.1, 4.3 Hz), 0.39-0.46 (2H, m), 0.99-1.11 (1H, m), 2.56 (2H, t, J=6.2 Hz), 2.69-2.76 (4H, m), 3.12 (2H, t, J=6.4 Hz), 3.20 (2H, t, J=6.6 Hz), 3.53-3.64 (4H, m), 4.03 (2H, t, J=6.6 Hz), 4.49 (1H, t, J=5.4 Hz), 6.95 (1H, d, J=2.0 Hz), 7.06 (1H, dd, J=10.8, 2.0 Hz), 7.47-7.57 (2H, m), 7.58-7.67 (1H, m), 7.76-7.81 (1H, m), 8.51 (1H, t, J=6.0 Hz)

### Example number 1128

### 1-(3-Chlorophenyl)-N-((4,6-dimethylpyridin-3-yl)methyl)-6-(2-(2-hydroxyethyl)-1,2,3,4-tetrahydroisoquinolin-7-yl)-7-oxo-4,5,6,7-tetrahydro-1H-pyrazolo[3,4-c]pyridine-3-carboxamide

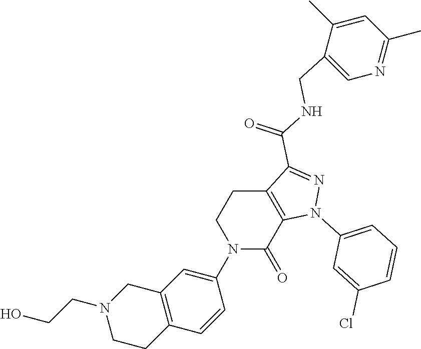 [SMILES: Cc1cc(C)c(CNC(=O)c2nn(-c3cccc(Cl)c3)c3c2CCN(c2ccc4c(c2)CN(CCO)CC4)C3=O)cn1]

### Ethyl 1-(3-chlorophenyl)-7-oxo-6-(1,2,3,4-tetrahydroisoquinolin-7-yl)-4,5-dihydropyrazolo[3,4-c]pyridine-3-carboxylate hydrochloride

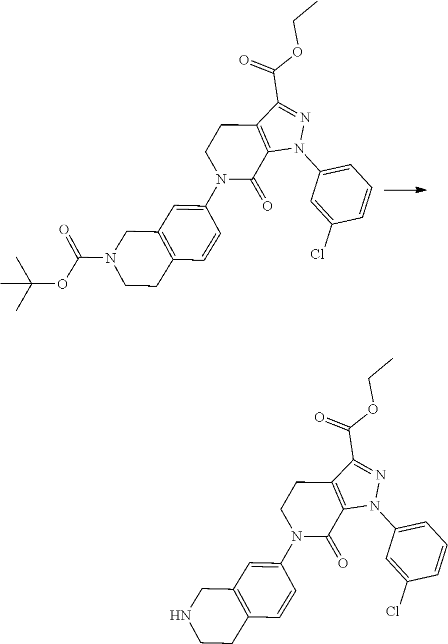 [SMILES: CCOC(=O)c1nn(-c2cccc(Cl)c2)c2c1CCN(c1ccc3c(c1)CN(C(=O)OC(C)(C)C)CC3)C2=O ; CCOC(=O)c1nn(-c2cccc(Cl)c2)c2c1CCN(c1ccc3c(c1)CNCC3)C2=O]

tert-Butyl 7-[1-(3-chlorophenyl)-3-ethoxycarbonyl-7-oxo-4,5-dihydropyrazolo[3,4-c]pyridin-6-yl]-3,4-dihydro-1H-isoquinoline-2-carboxylate (1200 mg, 2.2 mmol) was dissolved in 4M HCl in 1,4-dioxane (104 mL, 415 mmol). After 1 h at rt, Et₂O (100 mL) was added and the solid filtered off to afford the product (1040 mg, 98% yield).

[M+H]⁺=451.1

### Ethyl 1-(3-chlorophenyl)-6-[2-(2-hydroxyethyl)-3,4-dihydro-1H-isoquinolin-7-yl]-7-oxo-4,5-dihydropyrazolo[3,4-c]pyridine-3-carboxylate

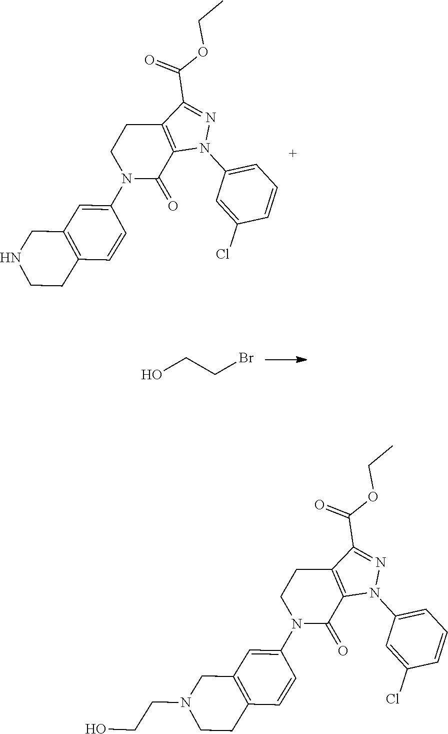

Ethyl 1-(3-chlorophenyl)-7-oxo-6-(1,2,3,4-tetrahydroisoquinolin-7-yl)-4,5-dihydropyrazolo[3,4-c] pyridine-3-carboxylate hydrochloride (1000 mg, 2.1 mmol) was dissolved in THF (30 mL). TEA (858 μL, 6.16 mmol) and 2-bromoethanol (308 mg, 2.46 mmol) were added. The reaction was stirred at rt for 18 h. The mixture was diluted with EtOAc (100 mL), washed with water (1×50 mL), brine (1×50 mL) dried (Na₂SO₄), filtered and evaporated. The residue was purified by flash chromatography (Silica, 6% MeOH in CHCl₃) to afford the product (488 mg, 48% yield).

[M+H]⁺=495.2

### 1-(3-Chlorophenyl)-6-[2-(2-hydroxyethyl)-3,4-dihydro-1H-isoquinolin-7-yl]-7-oxo-4,5-dihydropyrazolo[3,4-c]pyridine-3-carboxylic acid

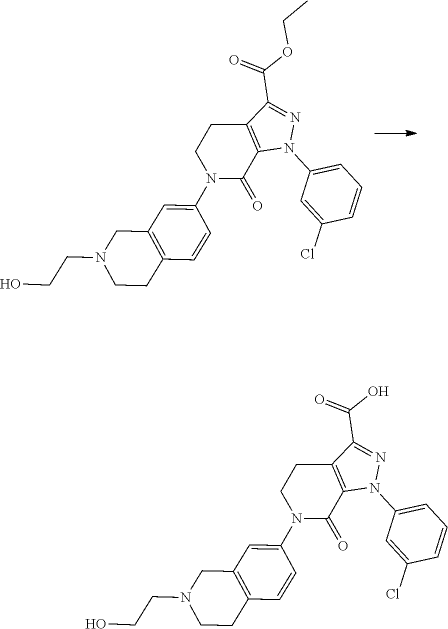 [SMILES: CCOC(=O)c1nn(-c2cccc(Cl)c2)c2c1CCN(c1ccc3c(c1)CN(CCO)CC3)C2=O ; O=C(O)c1nn(-c2cccc(Cl)c2)c2c1CCN(c1ccc3c(c1)CN(CCO)CC3)C2=O]

Ethyl 1-(3-chlorophenyl)-6-[2-(2-hydroxyethyl)-3,4-dihydro-1H-isoquinolin-7-yl]-7-oxo-4,5-dihydropyrazolo[3,4-c]pyridine-3-carboxylate (490 mg, 0.99 mmol) was dissolved in THF (100 mL) and water (10 mL). LiOH·H₂O (127 mg, 2.96 mmol) was added. The mixture was stirred at rt for 18 h. The THF was removed, the pH of the residue was adjusted to pH7 with 2M HCl and extracted with CHCl₃/IPA (4:1, 3×50 mL). The combined organic extracts were dried (Na₂SO₄) and evaporated to afford the product (430 mg, 93% yield).

[M+H]⁺=467.1

### 1-(3-Chlorophenyl)-N-((4,6-dimethylpyridin-3-yl)methyl)-6-(2-(2-hydroxyethyl)-1,2,3,4-tetrahydroisoquinolin-7-yl)-7-oxo-4,5,6,7-tetrahydro-1H-pyrazolo[3,4-c]pyridine-3-carboxamide

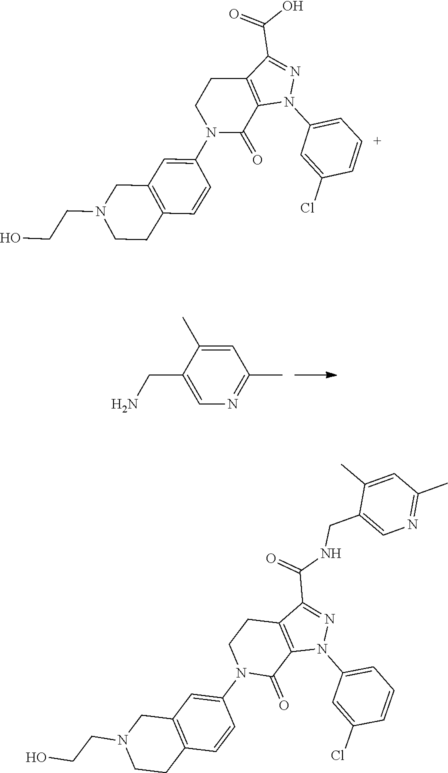

1-(3-Chlorophenyl)-6-[2-(2-hydroxyethyl)-3,4-dihydro-1H-isoquinolin-7-yl]-7-oxo-4,5-dihydropyrazolo[3,4-c]pyridine-3-carboxylic acid (68 mg, 0.14 mmol) and [benzotriazol-1-yloxy (dimethylamino)methylidene]-dimethylazanium (41 mg, 0.17 mmol) were dissolved in DCM (30 mL). TEA (60 μL, 0.43 mmol) was added. After 20 min (4,6-dimethylpyridin-3-yl) methanamine dihydrochloride (33 mg, 0.16 mmol) was added. The mixture was stirred at rt for 18 h after which time the mixture was diluted with CHCl₃ (100 mL). This solution was washed with sat. NaHCO₃(aq)(1×30 mL), water (1×30 mL), brine (1×30 mL), dried (Na₂SO₄) and evaporated. The residue was purified by flash chromatography (Silica, 10% MeOH in CHCl₃), further purified by automated prep HPLC (mass directed 2-60% MeCN in water with a basic mobile phase of 2.5% NH₄OH in water) and lyophilized from MeCN/water to afford the product (28 mg, 33% yield).

[M+H]⁺=585.3

¹H NMR (DMSO-d6, 400 MHZ) 2.31 (3H, s), 2.38 (3H, s), 2.54 (2H, t, J=6.2 Hz), 2.65-2.73 (2H, m), 2.75-2.82 (2H, m), 3.18 (2H, t, J=6.6 Hz), 3.51-3.61 (4H, m), 4.01 (2H, t, J=6.6 Hz), 4.43 (2H, d, J=5.9 Hz), 4.46 (1H, t, J=5.4 Hz), 7.03 (1H, s), 7.04 (1H, s), 7.07-7.13 (2H, m), 7.47-7.54 (2H, m), 7.62 (1H, dt, J=6.9, 2.0 Hz), 7.73-7.82 (1H, m), 8.30 (1H, s), 8.92 (1H, t, J=6.0 Hz)

### Example Number 1134

### 1-(3-Chlorophenyl)-6-(6-fluoro-2-(2-hydroxyethyl)-1,2,3,4-tetrahydroisoquinolin-7-yl)-N-((4-methylpyridin-3-yl)methyl)-7-oxo-4,5,6,7-tetrahydro-1H-pyrazolo[3,4-c]pyridine-3-carboxamide

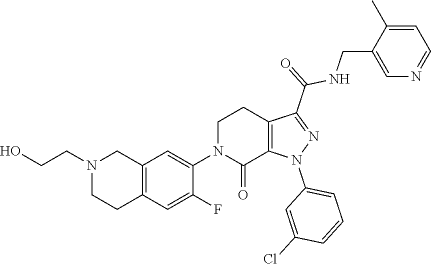 [SMILES: Cc1ccncc1CNC(=O)c1nn(-c2cccc(Cl)c2)c2c1CCN(c1cc3c(cc1F)CCN(CCO)C3)C2=O]

### 7-Bromo-6-fluoro-1,2,3,4-tetrahydroisoquinoline

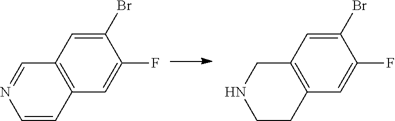 [SMILES: Fc1cc2ccncc2cc1Br ; Fc1cc2c(cc1Br)CNCC2]

Sodium triethylborohydride solution 1.0 M in THF (7.4 mL, 7.4 mmol) was added to a stirring solution of 7-bromo-6-fluoro-isoquinoline (420 mg, 1.86 mmol) in THF (15 mL) at 0° C. The mixture was stirred at 0° C. for 2 h. Water (10 mL) was added at 0° C. and the mixture was allowed to warm to rt with stirring. The solvent was removed, 1M HCl (aq)(20 mL) was added and the organics extracted into DCM. The aqueous layer was basified with sat. K₂CO₃ (aq)(20 mL), extracted with DCM and these DCM extracts were washed with brine, dried (Na₂SO₄) and evaporated to afford the product (470 mg, 100% yield).

[M+H]⁺=230.1, 232.0

¹H NMR (CDCl₃, 400 MHZ) δ 2.67-2.82 (2H, m), 3.10 (2H, t, J=6.0 Hz), 3.94 (2H, t, J=1.2 Hz), 6.77-6.88 (1H, m), 7.18 (1H, d, J=6.9 Hz)

### tert-Butyl 7-bromo-6-fluoro-3,4-dihydro-1H-isoquinoline-2-carboxylate

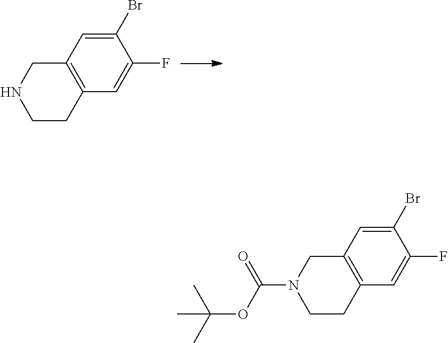 [SMILES: Fc1cc2c(cc1Br)CNCC2 ; CC(C)(C)OC(=O)N1CCc2cc(F)c(Br)cc2C1]

Boc₂O (580 mg, 2.66 mmol) was added to a stirring solution of 7-bromo-6-fluoro-1,2,3,4-tetrahydroisoquinoline (470 mg, 2.04 mmol) and TEA (710 μL, 5.11 mmol) in DCM (10 mL). The mixture was stirred at rt for 18 h. Water (10 mL) was added, the organic phase was separated, and the aqueous phase was extracted with DCM. The combined organic extracts were washed with brine (20 mL), dried (Na₂SO₄) and evaporated. Flash column chromatography (silica, 0-80% EtOAc in iso-hexane) afforded the product (663 mg, 98% yield).

[M−Boc+H]⁺=230.1, 232.0

¹H NMR (CDCl₃, 400 MHZ) δ 1.48 (9H, s), 2.73-2.81 (2H, m), 3.62 (2H, t, J=5.9 Hz), 4.51 (2H, s), 6.90 (1H, d, J=9.0 Hz), 7.24-7.32 (1H, m)

### tert-Butyl 7-[1-(3-chlorophenyl)-3-ethoxycarbonyl-7-oxo-4,5-dihydropyrazolo[3,4-c]pyridin-6-yl]-6-Fluoro-3,4-dihydro-1H-isoquinoline-2-carboxylate

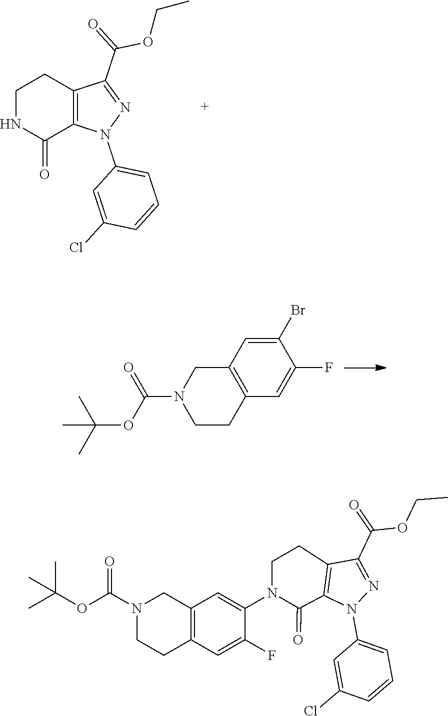 [SMILES: CCOC(=O)c1nn(-c2cccc(Cl)c2)c2c1CCNC2=O ; CC(C)(C)OC(=O)N1CCc2cc(F)c(Br)cc2C1 ; CCOC(=O)c1nn(-c2cccc(Cl)c2)c2c1CCN(c1cc3c(cc1F)CCN(C(=O)OC(C)(C)C)C3)C2=O]

Cu(I)I (63 mg, 0.33 mmol) was added to a N₂ (g) sparged stirring suspension of ethyl 1-(3-chlorophenyl)-7-oxo-5,6-dihydro-4H-pyrazolo[3,4-c]pyridine-3-carboxylate (530 mg, 1.66 mmol), tert-butyl 7-bromo-6-fluoro-3,4-dihydro-1H-isoquinoline-2-carboxylate (657 mg, 1.99 mmol), (1S,2S)—N,N′-dimethyl-1,2-cyclohexane-diamine (94 mg, 0.66 mmol) and K₃PO₄ (880 mg, 4.14 mmol) in anhydrous 1,4-dioxane (11 mL). The sealed reaction vessel was stirred at 100° C. for 20 h. The mixture was allowed to cool to rt and filtered through Celite®, rinsing with EtOAc. The filtrate was washed with water and brine, dried (Na₂SO₄) and evaporated. Flash chromatography (silica, 0-80% EtOAc in iso-hexane) afforded the product (387 mg, 38% yield).

[M−^{t}Bu+H]⁺=513.2, 515.2

¹H NMR (CDCl₃, 400 MHZ) δ 1.44 (3H, t, J=7.1 Hz), 1.47 (9H, s), 2.80 (2H, t, J=5.8 Hz), 3.34 (2H, t, J=6.7 Hz), 3.59-3.63 (2H, m), 4.02 (2H, t, J=6.7 Hz), 4.47 (2H, q, J=7.0 Hz), 4.50-4.53 (2H, m), 6.95 (1H, d, J=10.5 Hz), 7.06 (1H, d, J=7.4 Hz), 7.28-7.41 (2H, m), 7.46-7.53 (1H, m), 7.60-7.66 (1H, m)

### Ethyl 1-(3-chlorophenyl)-6-(6-fluoro-1,2,3,4-tetrahydroisoquinolin-7-yl)-7-oxo-4,5-dihydropyrazolo[3,4-c]pyridine-3-carboxylate hydrochloride

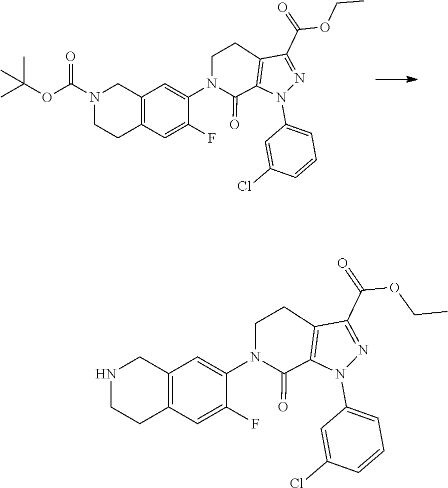 [SMILES: CCOC(=O)c1nn(-c2cccc(Cl)c2)c2c1CCN(c1cc3c(cc1F)CCN(C(=O)OC(C)(C)C)C3)C2=O ; CCOC(=O)c1nn(-c2cccc(Cl)c2)c2c1CCN(c1cc3c(cc1F)CCNC3)C2=O]

### HCl

4M HCl in 1,4-dioxane (3.0 mL, 12.0 mmol) was added to a solution of tert-butyl 7-[1-(3-chlorophenyl)-3-ethoxycarbonyl-7-oxo-4,5-dihydropyrazolo[3,4-c]pyridin-6-yl]-6-fluoro-3,4-dihydro-1H-isoquinoline-2-carboxylate (390 mg, 0.63 mmol) in 1,4-dioxane (3 mL). The mixture was then stirred at rt for 4 h. The solvent was evaporated to afford the product (334 mg, 100% yield).

[M+H]⁺=469.3, 471.3

¹H NMR (DMSO-d6, 400 MHZ) δ 1.33 (3H, t, J=7.1 Hz), 3.01 (2H, t, J=6.3 Hz), 3.18-3.31 (2H, m), 3.33-3.42 (2H, m), 4.00 (2H, t, J=6.6 Hz), 4.23 (2H, s), 4.36 (2H, q, J=7.1 Hz), 7.23 (1H, d, J=10.8 Hz), 7.36 (1H, d, J=7.5 Hz), 7.48-7.59 (2H, m), 7.58 (1H, dd, J=7.4, 1.8 Hz), 7.73 (1H, td, J=1.9, 0.5 Hz), 9.34 (2H, s)

### Ethyl 1-(3-chlorophenyl)-6-[6-fluoro-2-(2-hydroxyethyl)-3,4-dihydro-1H-isoquinolin-7-yl]-7-oxo-4,5-dihydropyrazolo[3,4-c]pyridine-3-carboxylate

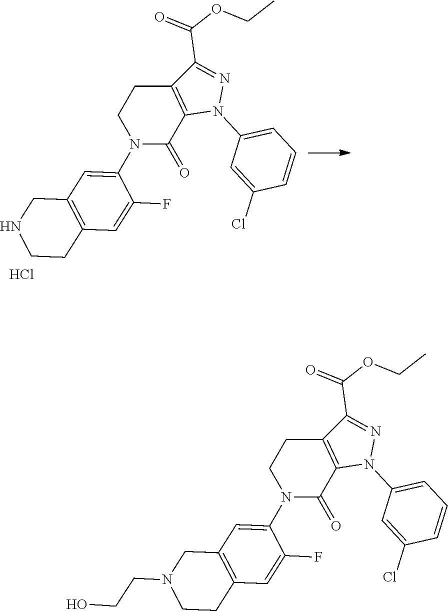 [SMILES: CCOC(=O)c1nn(-c2cccc(Cl)c2)c2c1CCN(c1cc3c(cc1F)CCNC3)C2=O ; Cl ; CCOC(=O)c1nn(-c2cccc(Cl)c2)c2c1CCN(c1cc3c(cc1F)CCN(CCO)C3)C2=O]

2-Bromoethanol (94 μL, 1.3 mmol) was added to a solution of ethyl 1-(3-chlorophenyl)-6-(6-fluoro-1,2,3,4-tetrahydroisoquinolin-7-yl)-7-oxo-4,5-dihydropyrazolo[3,4-c]pyridine-3-carboxylate hydrochloride (334 mg, 0.66 mmol) and TEA (370 μL, 2.64 mmol) in DMF (4 mL). The mixture was stirred at rt for 24 h. The mixture was quenched with sat. K₂CO₃ (aq)(20 mL) and extracted with EtOAc. The combined organic extracts were washed with brine (15 mL), dried over (Na₂SO₄) and evaporated. The residue was purified by SCX-2 ion-exchange, loaded with MeOH (20 mL) and eluted with 2M NH₃ in MeOH (20 mL), to afford the product (246 mg, 71% yield).

[M+H]⁺=513.2, 515.2

¹H NMR (DMSO-d6, 400 MHZ) δ 1.33 (3H, t, J=7.1 Hz), 2.54-2.60 (2H, m), 2.68 (2H, t, J=5.5 Hz), 2.80 (2H, t, J=5.9 Hz), 3.20 (2H, t, J=6.7 Hz), 3.55 (2H, s), 3.52-3.61 (2H, m), 3.99 (2H, t, J=6.6 Hz), 4.35 (2H, q, J=7.1 Hz), 4.47 (1H, t, J=5.4 Hz), 7.04 (1H, d, J=11.1 Hz), 7.14 (1H, d, J=7.7 Hz), 7.48-7.57 (1H, m), 7.53-7.61 (2H, m), 7.69-7.75 (1H, m)

### 1-(3-Chlorophenyl)-6-[6-fluoro-2-(2-hydroxyethyl)-3,4-dihydro-1H-isoquinolin-7-yl]-7-oxo-4,5-dihydropyrazolo[3,4-c]pyridine-3-carboxylic acid

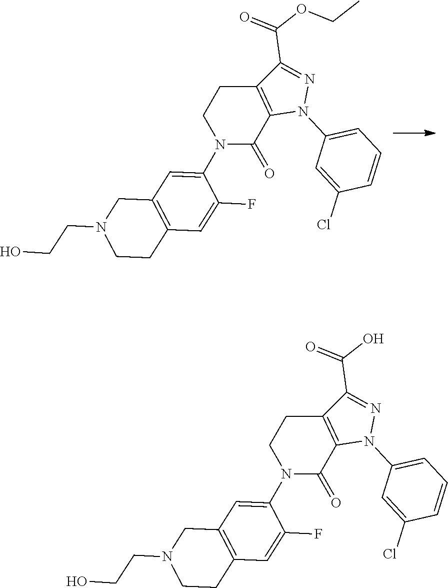 [SMILES: CCOC(=O)c1nn(-c2cccc(Cl)c2)c2c1CCN(c1cc3c(cc1F)CCN(CCO)C3)C2=O ; O=C(O)c1nn(-c2cccc(Cl)c2)c2c1CCN(c1cc3c(cc1F)CCN(CCO)C3)C2=O]

To a solution of ethyl 1-(3-chlorophenyl)-6-[6-fluoro-2-(2-hydroxyethyl)-3,4-dihydro-1H-isoquinolin-7-yl]-7-oxo-4,5-dihydropyrazolo[3,4-c]pyridine-3-carboxylate (246 mg, 0.470 mmol) in a mixture of THF (3 mL) and water (1.5 mL), was added LiOH·H₂O (30 mg, 0.71 mmol), and the mixture was stirred at rt for 18 h. The THF was removed and the remaining aqueous solution was acidified to pH=2-3 using 1M HCl (aq). The acidified aqueous phase was extracted with EtOAc and the combined organic extracts were dried (Na₂SO₄) and evaporated to afford the product (268 mg, 100% yield).

[M+H]⁺=485.1, 485.2

¹H NMR (DMSO-d6, 400 MHZ) δ 2.58 (2H, t, J=6.1 Hz), 2.72 (2H, t, J=5.8 Hz), 2.81 (2H, d, J=5.8 Hz), 3.18 (2H, t, J=6.7 Hz), 3.55-3.62 (4H, m), 3.95 (2H, t, J=6.5 Hz), 7.04 (1H, d, J=10.9 Hz), 7.15 (1H, d, J=7.7 Hz), 7.46-7.61 (3H, m), 7.69 (1H, dt, J=2.4, 1.0 Hz)

### 1-(3-Chlorophenyl)-6-(6-fluoro-2-(2-hydroxyethyl)-1,2,3,4-tetrahydroisoquinolin-7-yl)-N-((4-methylpyridin-3-yl)methyl)-7-oxo-4,5,6,7-tetrahydro-1H-pyrazolo[3,4-c]pyridine-3-carboxamide

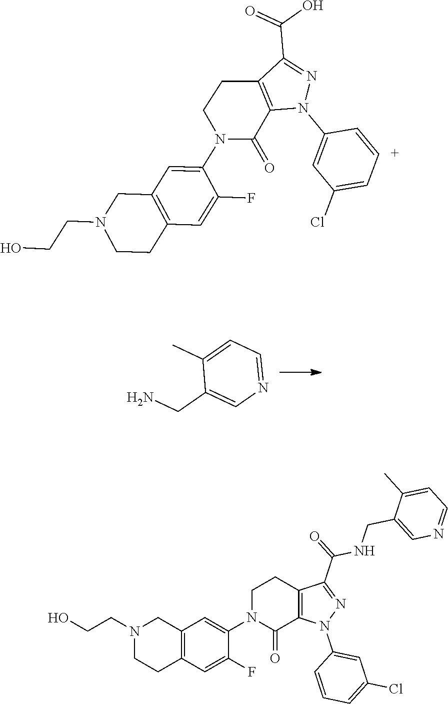

O-(7-Azabenzotriazol-1-yl)-N,N,N′,N′-tetramethyluronium hexafluorophosphate (120 mg, 0.32 mmol) was added to a solution of 1-(3-chlorophenyl)-6-[6-fluoro-2-(2-hydroxyethyl)-3,4-dihydro-1H-isoquinolin-7-yl]-7-oxo-4,5-dihydropyrazolo[3,4-c]pyridine-3-carboxylic acid (85 mg, 0.18 mmol), (4-methylpyridin-3-yl) methanamine (32 mg, 0.26 mmol) and DIPEA (140 μL, 0.79 mmol) in DMF (1.5 mL). The mixture was stirred at rt for 24 h. Sat. K₂CO₃ (aq)(10 mL) was added and extracted with EtOAc (3×5 mL). The combined organic extracts were washed with brine, dried (Na₂SO₄) and evaporated. Flash chromatography (silica, 0 -10% MeOH in DCM), followed by SCX-2 ion exchange-loaded with MeOH (10 mL) and eluting with 3M NH₃ in MeOH (10 mL) afforded the product (24 mg, 23% yield).

[M+H]⁺=589.2

¹H NMR (DMSO-d6, 400 MHZ) δ 2.36 (3H, s), 2.45-2.57 (2H, m), 2.68-2.73 (1H, m), 2.79-2.84 (2H, m), 3.21 (2H, t, J=6.6 Hz), 3.26-3.37 (2H, m), 3.56-3.60 (4H, m), 3.96 (2H, t, J=6.6 Hz), 4.48 (2H, d, J=6.0 Hz), 7.05 (1H, d, J=11.0 Hz), 7.12-7.22 (2H, m), 7.46-7.57 (2H, m), 7.57-7.65 (1H, m), 7.74-7.80 (1H, m), 8.32 (1H, d, J=4.9 Hz), 8.44 (1H, s), 9.01 (1H, t, J=6.0 Hz)

### Example Number 1144

### 1-(3-Chlorophenyl)-6-(2′-(2-hydroxyethyl)-2′,3′-dihydro-1′H-spiro[cyclopropane-1,4′-isoquinolin]-7′-yl)-N-((4-methylpyridin-3-yl)methyl)-7-oxo-4,5,6,7-tetrahydro-1H-pyrazolo[3,4-c]pyridine-3-carboxamide

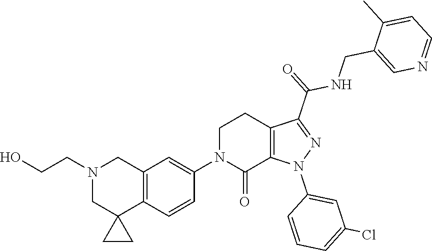 [SMILES: Cc1ccncc1CNC(=O)c1nn(-c2cccc(Cl)c2)c2c1CCN(c1ccc3c(c1)CN(CCO)CC31CC1)C2=O]

### N-[[1-(4-Bromophenyl)cyclopropyl]methyl]-2,2,2-trifluoro-acetamide

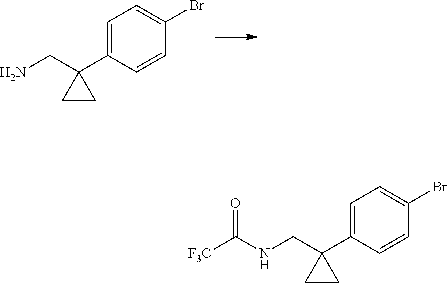 [SMILES: NCC1(c2ccc(Br)cc2)CC1 ; O=C(NCC1(c2ccc(Br)cc2)CC1)C(F)(F)F]

To a solution of [1-(4-bromophenyl)cyclopropyl]methanamine (650 mg, 2.87 mmol) in DCM (30 mL), was added TEA (1.2 mL, 8.62 mmol) and trifluoroacetic anhydride (0.60 mL, 4.31 mmol), and the mixture was stirred for rt for 20 h. The solution was diluted with DCM (50 mL) and washed with sat. NaHCO₃(aq) and brine, dried (Na₂SO₄) and evaporated. Flash chromatography (silica, 0-50% EtOAc in iso-hexane) afforded the product (881 mg, 95% yield).

[M+H]⁺=320.0, 322.0

¹H NMR (DMSO-d6, 400 MHZ) δ 0.73-0.82 (2H, m), 0.90-0.98 (2H, m), 3.40 (2H, d, J=5.9 Hz), 7.18-7.27 (2H, m), 7.42-7.51 (2H, m), 9.45 (1H, t, J=5.9 Hz)

### 1-(7-Bromospiro[1,3-dihydroisoquinoline-4,1′-cyclopropane]-2-yl)-2,2,2-trifluoro-ethanone

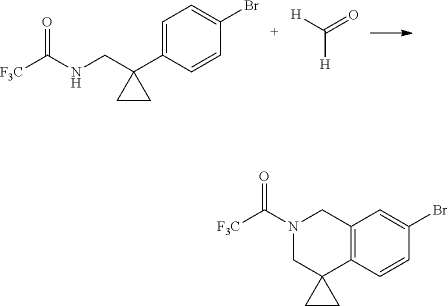

N-[[1-(4-bromophenyl)cyclopropyl]methyl]-2,2,2-trifluoro-acetamide (875 mg, 2.72 mmol) and paraformaldehyde (408 mg, 13.6 mmol) were added to a mixture of AcOH (5 mL) and sulfuric acid (3.5 mL) and the mixture was stirred at rt for 20 h. The mixture was poured into cold water (100 mL) and extracted with EtOAc (2×75 mL). The combined organic extracts were washed with sat. NaHCO₃(aq) and brine, dried (Na₂SO₄) and evaporated. Flash chromatography (silica, 0-30% EtOAc in iso-hexane) afforded the product (524 mg, 58% yield).

¹H NMR (DMSO-d6, 400 MHZ) δ 0.98-1.08 (4H, m), 3.66 (2H, d, J=9.9 Hz), 4.89 (2H, d, J=11.1 Hz), 6.83 (1H, dd, J=8.5, 1.9 Hz), 7.33-7.40 (1H, m), 7.54 (1H, dd, J=15.2, 1.7 Hz)

### 7-Bromospiro[2,3-dihydro-1H-isoquinoline-4,1′-cyclopropane]

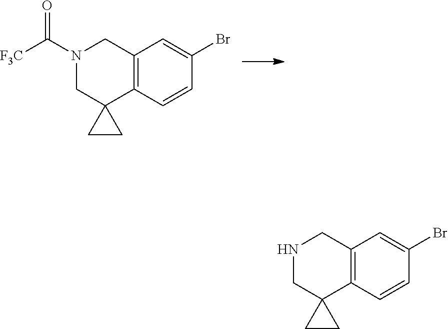 [SMILES: O=C(N1Cc2cc(Br)ccc2C2(CC2)C1)C(F)(F)F ; Brc1ccc2c(c1)CNCC21CC1]

To a solution of 1-(7-bromospiro[1,3-dihydroisoquinoline-4,1′-cyclopropane]-2-yl)-2,2,2-trifluoro-ethanone (520 mg, 1.56 mmol) in MeOH (15 mL) was added K₂CO₃ (430 mg, 3.11 mmol) and the mixture stirred at rt for 20 h and evaporated. The residue was partitioned between EtOAc (50 mL) and water (15 mL). The organic extract was washed with brine, dried (Na₂SO₄) and evaporated to afford the product (343 mg, 93% yield).

[M+H]⁺=238.1, 240.1

¹H NMR (DMSO-d6, 400 MHZ) δ 0.77-0.84 (2H, m), 0.87-0.95 (2H, m), 2.62-2.71 (1H, m), 2.72 (2H, s), 3.91 (2H, s), 6.67 (1H, d, J=8.4 Hz), 7.20 (1H, d, J=2.3 Hz), 7.21-7.24 (1H, m)

### tert-Butyl 7-bromospiro[1,3-dihydroisoquinoline-4,1′-cyclopropane]-2-carboxylate (163848)

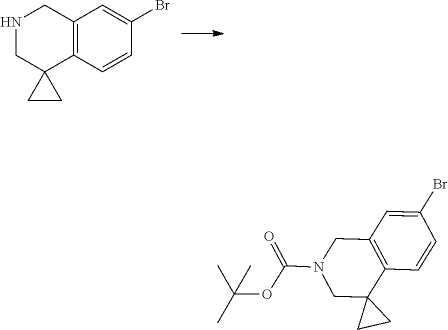 [SMILES: Brc1ccc2c(c1)CNCC21CC1 ; CC(C)(C)OC(=O)N1Cc2cc(Br)ccc2C2(CC2)C1]

To a solution of 7-bromospiro[2,3-dihydro-1H-isoquinoline-4,1′-cyclopropane] (340 mg, 1.43 mmol) in DCM (15 mL) were added TEA (0.40 mL, 2.9 mmol) and Boc₂O (436 mg, 2.00 mmol) and the mixture stirred at rt for 20 h. The mixture was diluted with DCM (50 mL) and washed with sat. NaHCO₃(aq)(15 mL) and brine (15 mL), dried (Na₂SO₄) and evaporated. Flash chromatography (silica, 0-35% EtOAc in iso-hexane) afforded the product (359 mg, 74% yield). [M-Boc+H]+=238.1, 240.1 1H NMR (DMSO-d6, 400 MHZ) δ 0.95 (4H, d, J=5.0 Hz), 1.41 (9H, s), 3.39 (2H, s), 4.62 (2H, s), 6.77 (1H, d, J=8.4 Hz), 7.30 (1H, dd, J=8.4, 2.2 Hz), 7.39 (1H, d, J=2.2 Hz)

### tert-Butyl 7-[1-(3-chlorophenyl)-3-ethoxycarbonyl-7-oxo-4,5-dihydropyrazolo[3,4-c]pyridin-6-yl] spiro[1,3-dihydroisoquinoline-4,1′-cyclopropane]-2-carboxylate

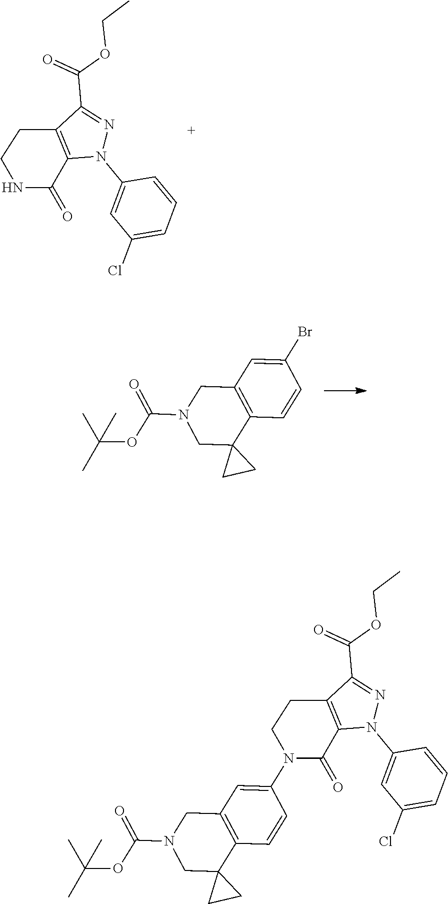 [SMILES: CCOC(=O)c1nn(-c2cccc(Cl)c2)c2c1CCNC2=O ; CC(C)(C)OC(=O)N1Cc2cc(Br)ccc2C2(CC2)C1 ; CCOC(=O)c1nn(-c2cccc(Cl)c2)c2c1CCN(c1ccc3c(c1)CN(C(=O)OC(C)(C)C)CC31CC1)C2=O]

A solution of ethyl 1-(3-chlorophenyl)-7-oxo-5,6-dihydro-4H-pyrazolo[3,4-c]pyridine-3-carboxylate (470 mg, 1.47 mmol) and tert-butyl 7-bromospiro[1,3-dihydroisoquinoline-4,1′-cyclopropane]-2-carboxylate (355 mg, 1.05 mmol) in dry 1,4-dioxane (10 mL) was purged with N₂ (g) for 5 min. Cs₂CO₃ (684 mg, 2.10 mmol), XantPhos (121 mg, 0.210 mmol) and Pd₂(dba)₃ (96 mg, 0.11 mmol) were added. The sealed vessel was stirred at 100° C. for 20 h. The mixture was filtered through Celite® and rinsed with EtOAc (100 ml). The filtrate was washed with water and brine, dried (Na₂SO₄) and evaporated. Flash chromatography (silica, 0-50% EtOAc in iso-hexane) afforded the product (279 mg, 46% yield).

[M−^{t}Bu+H]⁺=521.2, 523.2

¹H NMR (DMSO-d6, 400 MHZ) δ 0.93-0.98 (4H, m), 1.33 (3H, t, J=7.1 Hz), 1.41 (9H, s), 3.19 (2H, t, J=6.7 Hz), 3.41 (2H, s), 4.05 (2H, t, J=6.7 Hz), 4.35 (2H, q, J=7.1 Hz), 4.61 (2H, s), 6.84 (1H, d, J=8.3 Hz), 7.10-7.17 (2H, m), 7.49-7.56 (2H, m), 7.58 (1H, dt, J=7.3, 1.9 Hz), 7.72-7.75 (1H, m)

### Ethyl 1-(3-chlorophenyl)-7-oxo-6-spiro[2,3-dihydro-1H-isoquinoline-4,1′-cyclopropane]-7-yl-4,5-dihydropyrazolo[3,4-c]pyridine-3-carboxylate hydrochloride

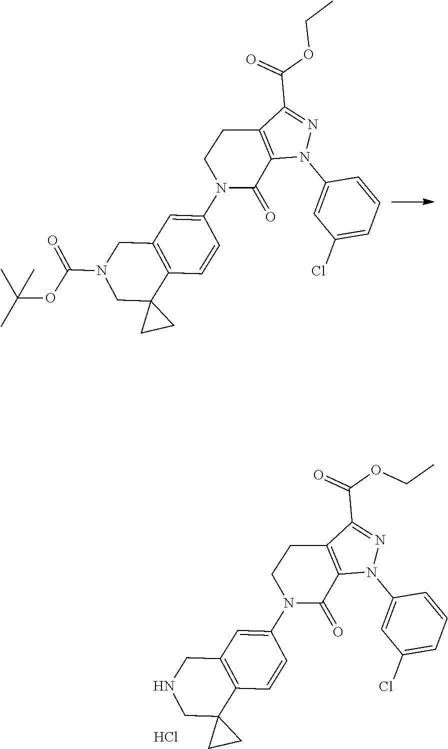 [SMILES: CCOC(=O)c1nn(-c2cccc(Cl)c2)c2c1CCN(c1ccc3c(c1)CN(C(=O)OC(C)(C)C)CC31CC1)C2=O ; Cl ; CCOC(=O)c1nn(-c2cccc(Cl)c2)c2c1CCN(c1ccc3c(c1)CNCC31CC1)C2=O]

To a solution of tert-butyl 7-[1-(3-chlorophenyl)-3-ethoxycarbonyl-7-oxo-4,5-dihydropyrazolo[3,4-c]pyridin-6-yl] spiro[1,3-dihydroisoquinoline-4,1′-cyclopropane]-2-carboxylate (28 mg, 0.48 mmol) in 1,4-dioxane (2.5 mL) was added 4M HCl in 1,4-dioxane (2.5 mL) and the mixture stirred at rt for 3 h. The solvent was evaporated to afford the product (250 mg, 100% yield).

[M+H]⁺=477.2, 479.2

¹H NMR (DMSO-d6, 400 MHz) δ 1.06-1.16 (4H, m), 1.33 (3H, t, J=7.1 Hz), 3.20 (2H, t, J=6.6 Hz), 3.25 (2H, s), 4.05 (2H, t, J=6.6 Hz), 4.28-4.41 (4H, m), 6.90 (1H, d, J=8.4 Hz), 7.21-7.27 (2H, m), 7.48-7.57 (2H, m), 7.59 (1H, dt, J=7.4, 1.8 Hz), 7.71-7.76 (1H, m), 9.43 (2H, s br)

### Ethyl 1-(3-chlorophenyl)-6-[2-(2-hydroxyethyl) spiro[1,3-dihydroisoquinoline-4,1′-cyclopropane]-7-yl]-7-oxo-4,5-dihydropyrazolo[3,4-c]pyridine-3-carboxylate

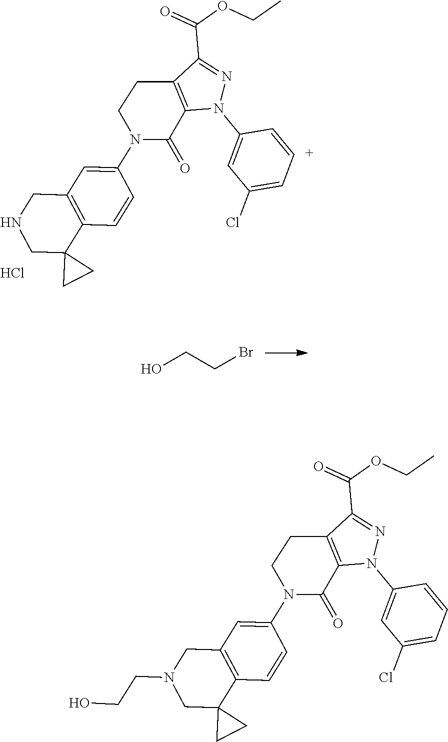

To a solution of ethyl 1-(3-chlorophenyl)-7-oxo-6-spiro[2,3-dihydro-1H-isoquinoline-4,1′-cyclopropane]-7-yl-4,5-dihydropyrazolo[3,4-c]pyridine-3-carboxylate hydrochloride (250 mg, 0.49 mmol) in DMF (5 mL) were added TEA (240 μL, 1.70 mmol) and 2-bromoethanol (69 μL, 0.97 mmol) and the mixture stirred at rt for 20 h. The solution was diluted with EtOAc (50 mL) and washed with sat. NaHCO₃(aq) and brine, dried over (Na₂SO₄) and evaporated. Ion-exchange with SCX-2 column, washing with MeOH (10 mL) and eluting with 3M NH₃ in MeOH (10 mL) afforded the product (190 mg, 75% yield).

[M+H]⁺=521.1, 523.1

¹H NMR (DMSO-d6, 400 MHZ) δ 0.82-0.89 (2H, m), 0.89-0.99 (2H, m), 1.33 (3H, t, J=7.1 Hz), 2.51-2.57 (4H, m), 3.15-3.24 (2H, m), 3.55 (2H, q, J=6.1 Hz), 3.69 (2H, s), 4.03 (2H, t, J=6.7 Hz), 4.35 (2H, q, J=7.1 Hz), 4.45 (1H, t, J=5.4 Hz), 6.72 (1H, d, J=8.4 Hz), 7.02 (1H, d, J=2.2 Hz), 7.08 (1H, dd, J=8.3, 2.3 Hz), 7.48-7.56 (2H, m), 7.58 (1H, dt, J=7.3, 1.8 Hz), 7.73 (1H, t, J=2.0 Hz)

### 1-(3-Chlorophenyl)-6-[2-(2-hydroxyethyl) spiro[1,3-dihydroisoquinoline-4,1′-cyclopropane]-7-yl]-7-oxo-4,5-dihydropyrazolo[3,4-c]pyridine-3-carboxylic acid

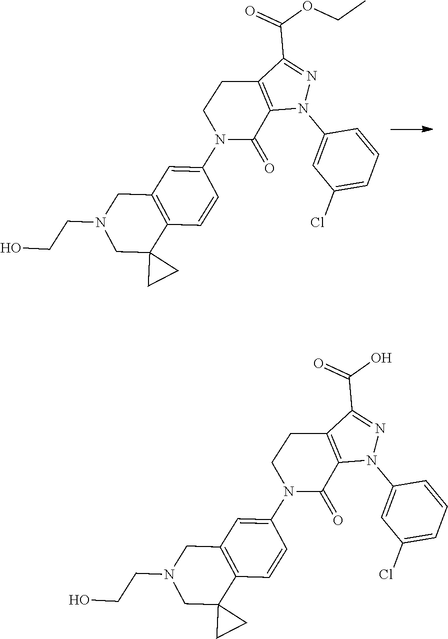 [SMILES: CCOC(=O)c1nn(-c2cccc(Cl)c2)c2c1CCN(c1ccc3c(c1)CN(CCO)CC31CC1)C2=O ; O=C(O)c1nn(-c2cccc(Cl)c2)c2c1CCN(c1ccc3c(c1)CN(CCO)CC31CC1)C2=O]

To a solution of ethyl 1-(3-chlorophenyl)-6-[2-(2-hydroxyethyl) spiro[1,3-dihydroisoquinoline-4,1′-cyclopropane]-7-yl]-7-oxo-4,5-dihydropyrazolo[3,4-c]pyridine-3-carboxylate (190 mg, 0.37 mmol) in a mixture of THF (3 mL) and water (1.5 mL) was added LiOH·H₂O (23 mg, 0.55 mmol) and the mixture stirred at rt for 18 h. The THF was removed and the aqueous solution was acidified to pH=2-3 using 1M HCl (aq).

The acidified aqueous phase was extracted with EtOAc (3×40 mL), the combined organic extracts were dried (Na₂SO₄) and evaporated to afford the product (213 mg, 100% yield).

[M+H]⁺=493.2, 495.2

¹H NMR (DMSO-d6, 400 MHz) δ 0.88-0.95 (2H, m), 0.95-1.00 (2H, m), 2.67-2.74 (2H, m), 2.75-2.79 (2H, m), 3.17 (2H, t, J=6.6 Hz), 3.57-3.67 (3H, m), 3.89 (2H, s), 4.02 (2H, t, J=6.5 Hz), 6.75 (1H, d, J=8.4 Hz), 7.06 (1H, d, J=2.2 Hz), 7.12 (1H, dd, J=8.4, 2.2 Hz), 7.48-7.55 (2H, m), 7.58 (1H, dt, J=6.8, 2.1 Hz), 7.72 (1H, t, J=1.9 Hz)

### 1-(3-Chlorophenyl)-6-(2′-(2-hydroxyethyl)-2′,3′-dihydro-1′H-spiro[cyclopropane-1,4′-isoquinolin]-7′-yl)-N-((4-methylpyridin-3-yl)methyl)-7-oxo-4,5,6,7-tetrahydro-1H-pyrazolo[3,4-c]pyridine-3-carboxamide

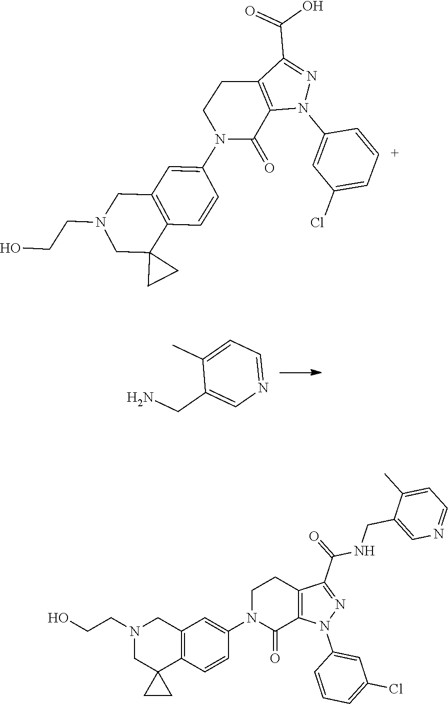

To a solution of 1-(3-chlorophenyl)-6-[2-(2-hydroxyethyl) spiro[1,3-dihydroisoquinoline-4,1′-cyclopropane]-7-yl]-7-oxo-4,5-dihydropyrazolo[3,4-c]pyridine-3-carboxylic acid (60 mg, 0.12 mmol) in DMF (1.5 mL) were added (4-methylpyridin-3-yl) methanamine (30 mg, 0.24 mmol), DIPEA (110 μL, 0.61 mmol) and HATU (69 mg, 0.18 mmol), and the mixture stirred at rt for 72 h. The mixture was diluted with EtOAc (75 mL) and washed with sat. NaHCO₃(aq) and brine, dried (Na₂SO₄) and evaporated. Flash chromatography (silica, 0-9% MeOH in DCM) followed by ion-exchange SCX-2 column, washing with MeOH (10 mL) and eluting with 3M NH₃ in MeOH (10 mL) afforded the product (42 mg, 55% yield).

[M+H]⁺=597.3, 599.3

¹H NMR ((DMSO-d6, 400 MHZ) δ 0.86 (2H, t, J=4.1 Hz), 0.91 (2H, t, J=4.3 Hz), 2.36 (3H, s), 2.52-2.58 (4H, m), 3.19 (2H, t, J=6.6 Hz), 3.56 (2H, q, J=6.0 Hz), 3.69 (2H, s), 4.01 (2H, t, J=6.6 Hz), 4.44 (1H, t, J=5.4 Hz), 4.47 (2H, d, J=6.0 Hz), 6.71 (1H, d, J=8.5 Hz), 7.02 (1H, d, J=2.2 Hz), 7.08 (1H, dd, J=8.3, 2.2 Hz), 7.18 (1H, d, J=4.9 Hz), 7.45-7.58 (2H, m), 7.62 (1H, dt, J=6.8, 2.1 Hz), 7.75-7.80 (1H, m), 8.32 (1H, d, J=4.9 Hz), 8.43 (1H, s), 8.98 (1H, t, J=6.0 Hz)

### Example Number 1148

### (R)-1-(3-Chlorophenyl)-6-(2-(2-hydroxyethyl)-4-methyl-1,2,3,4-tetrahydroisoquinolin-7-yl)-N-((4-methylpyridin-3-yl)methyl)-7-oxo-4,5,6,7-tetrahydro-1H-pyrazolo[3,4-c]pyridine-3-carboxamide

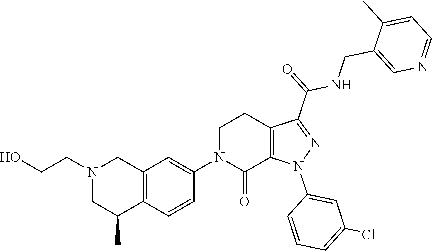 [SMILES: Cc1ccncc1CNC(=O)c1nn(-c2cccc(Cl)c2)c2c1CCN(c1ccc3c(c1)CN(CCO)C[C@@H]3C)C2=O]

### (2R)-2-(4-Bromophenyl)propan-1-amine hydrochloride

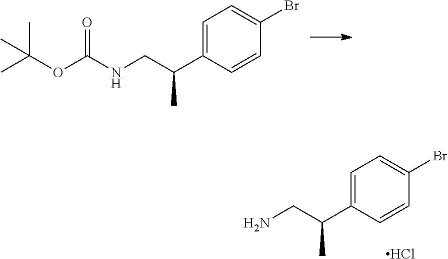 [SMILES: C[C@@H](CNC(=O)OC(C)(C)C)c1ccc(Br)cc1 ; C[C@@H](CN)c1ccc(Br)cc1 ; Cl]

To a solution of (R)-tert-butyl (2-(4-bromophenyl) propyl)carbamate (1.00 g, 3.18 mmol) in 1,4-dioxane (10 mL) was added 4M HCl in 1,4-dioxane (15 mL) and the mixture stirred at rt for 72 h. The solvent was evaporated to afford the product (836 mg, 100% yield). [M+H]⁺=214.1, 216.1

### N-[(2R)-2-(4-Bromophenyl) propyl]-2,2,2-trifluoro-acetamide

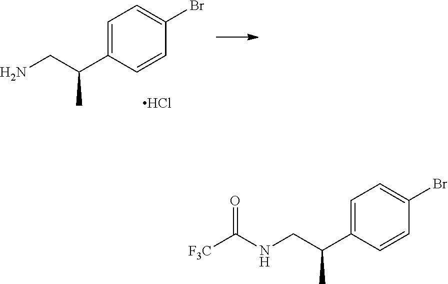 [SMILES: C[C@@H](CN)c1ccc(Br)cc1 ; Cl ; C[C@@H](CNC(=O)C(F)(F)F)c1ccc(Br)cc1]

To a solution of (2R)-2-(4-bromophenyl)propan-1-amine hydrochloride (800 mg, 3.19 mmol) in DCM (25 mL) was added TEA (1.2 mL, 8.62 mmol) and trifluoroacetic anhydride (0.60 mL, 4.31 mmol) and the mixture was stirred at rt for 20 h. The mixture was diluted with DCM (50 mL) and washed with sat. NaHCO₃(aq) and brine, dried (Na₂SO₄) and evaporated. Flash chromatography (silica, 0-50% EtOAc in iso-hexane) afforded the product (935 mg, 94% yield).

[M+H]⁺=308.0, 310.0

¹H NMR (DMSO-d6, 400 MHZ) δ 1.18 (3H, d, J=7.0 Hz), 2.99 (1H, h, J=7.2 Hz), 3.30-3.34 (2H, m), 7.19 (2H, d, J=8.4 Hz), 7.49 (2H, d, J=8.5 Hz), 9.43 (1H, t, J=5.7 Hz)

### 2,2,2-Trifluoro-1-[(4R)-7-bromo-4-methyl-3,4-dihydro-1H-isoquinolin-2-yl]ethanone

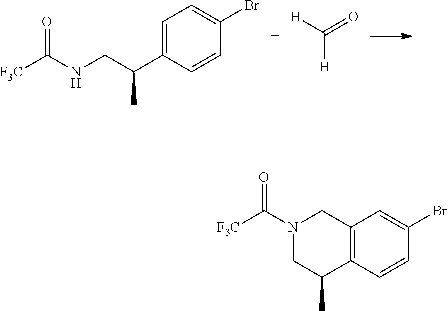

A cold mixture of AcOH (6 mL) and sulfuric acid (3.8 mL) was added to 2,2,2-trifluoro-N-[(2R)-2-(4-bromophenyl) propyl] acetamide (930 mg, 3.00 mmol) followed by paraformaldehyde (630 mg, 21.0 mmol). The mixture was stirred at rt for 48 h, poured into cold water (100 mL) and extracted with EtOAc (2×75 mL). The combined organic extracts were washed with sat. NaHCO₃(aq) and brine, dried (Na₂SO₄) and evaporated. Flash chromatography (silica, 0-30% EtOAc in iso-hexane) afforded the product (806 mg, 83% yield).

¹H NMR (DMSO-d6, 400 MHZ) δ 1.18 (3H, dd, J=8.6, 7.0 Hz), 3.06 (1H, h, J=6.5 Hz), 3.58-3.71 (1H, m), 3.72-3.83 (1H, m), 4.54-4.79 (1H, m), 4.85 (1H, d, J=17.5 Hz), 7.25 (1H, d, J=8.3 Hz), 7.35-7.48 (1H, m), 7.54 (1H, dd, J=14.6, 2.2 Hz)

### (4R)-7-Bromo-4-methyl-1,2,3,4-tetrahydroisoquinoline

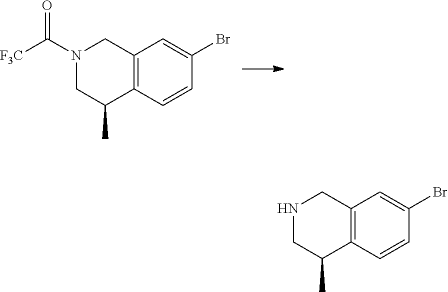 [SMILES: C[C@H]1CN(C(=O)C(F)(F)F)Cc2cc(Br)ccc21 ; C[C@H]1CNCc2cc(Br)ccc21]

To a solution of 2,2,2-trifluoro-1-[(4R)-7-bromo-4-methyl-3,4-dihydro-1H-isoquinolin-2-yl]ethanone (800 mg, 2.48 mmol) in MeOH (25 mL) was added K₂CO₃ (687 mg, 4.97 mmol) and the mixture was stirred at rt for 20 h and evaporated The residue was partitioned between EtOAc (50 mL) and water (15 mL). The organic extract was washed with brine, dried (Na₂SO₄) and evaporated to afford the product (504 mg, 90% yield).

[M+H]⁺=226.1, 228.1

¹H NMR (DMSO-d6, 400 MHZ) δ 1.18 (3H, d, J=7.0 Hz), 2.60 (1H, dd, J=12.4, 5.8 Hz), 2.72 (1H, h, J=6.6 Hz), 2.98 (1H, dd, J=12.4, 4.9 Hz), 3.78 (1H, d, J=16.6 Hz), 3.83 (1H, d, J=16.7 Hz), 7.15 (1H, d, J=8.3 Hz), 7.20 (1H, d, J=2.2 Hz), 7.28 (1H, dd, J=8.2, 2.2 Hz) tert-Butyl (4R)-7-bromo-4-methyl-3,4-dihydro-1H-isoquinoline-2-carboxylate

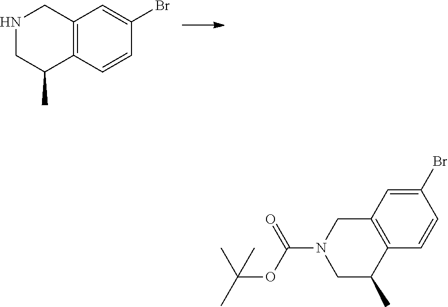 [SMILES: C[C@H]1CNCc2cc(Br)ccc21 ; C[C@H]1CN(C(=O)OC(C)(C)C)Cc2cc(Br)ccc21]

To a solution of (4R)-7-bromo-4-methyl-1,2,3,4-tetrahydroisoquinoline (340 mg, 1.43 mmol) in DCM (15 mL) were added TEA (0.40 mL, 2.86 mmol) and Boc₂O (436 mg, 2.00 mmol), and the mixture stirred at rt for 20 h. The mixture was diluted with DCM (50 mL) and washed with sat. NaHCO₃(aq) and brine, dried (Na₂SO₄) and evaporated. Flash chromatography (silica, 0-35% EtOAc in iso-hexane) afforded the product (359 mg, 74% yield).

[M−Boc+H]⁺=226.1, 228.1

¹H NMR (DMSO-d6, 400 MHz) δ 1.15 (3H, d, J=7.0 Hz), 1.42 (9H, s), 2.89 (1H, h, J=5.8 Hz), 3.41 (1H, dd, J=13.1, 4.3 Hz), 3.46-3.53 (1H, m), 4.27-4.49 (1H, m), 4.67 (1H, d, J=17.4 Hz), 7.19 (1H, d, J=8.3 Hz), 7.36 (1H, dd, J=8.3, 2.2 Hz), 7.41 (1H, d, J=2.1 Hz)

### tert-Butyl (4R)-7-[1-(3-chlorophenyl)-3-ethoxycarbonyl-7-oxo-4,5-dihydropyrazolo[3,4-c]pyridin-6-yl]-4-methyl-3,4-dihydro-1H-isoquinoline-2-carboxylate

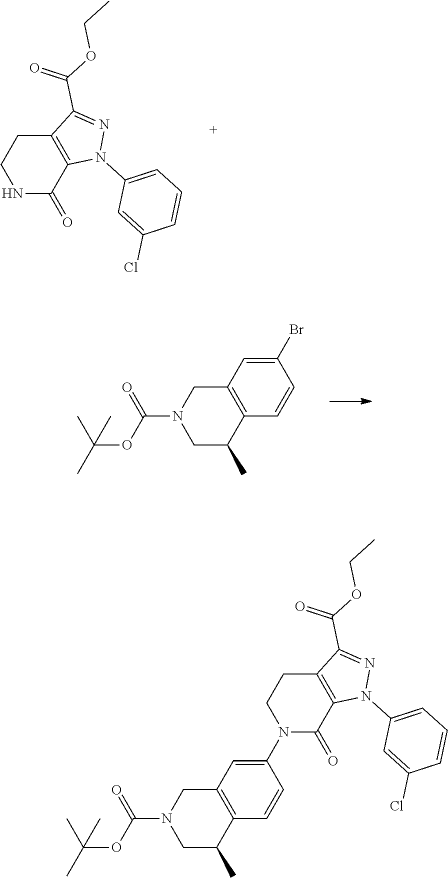 [SMILES: CCOC(=O)c1nn(-c2cccc(Cl)c2)c2c1CCNC2=O ; C[C@H]1CN(C(=O)OC(C)(C)C)Cc2cc(Br)ccc21 ; CCOC(=O)c1nn(-c2cccc(Cl)c2)c2c1CCN(c1ccc3c(c1)CN(C(=O)OC(C)(C)C)C[C@@H]3C)C2=O]

A solution of ethyl 1-(3-chlorophenyl)-7-oxo-5,6-dihydro-4H-pyrazolo[3,4-c]pyridine-3-carboxylate (676 mg, 2.12 mmol) and tert-butyl (4R)-7-bromo-4-methyl-3,4-dihydro-1H-isoquinoline-2-carboxylate (575 mg, 1.76 mmol) in 1,4-dioxane (15 mL) was purged with N₂ (g) for 5 min. Cs₂CO₃ (1150 mg, 3.53 mmol), XantPhos (102 mg, 0.18 mmol) and XantPhos Pd G4 (85 mg, 0.088 mmol) were added. The sealed reaction vessel was stirred at 100° C. for 20 h. The mixture was filtered through Celite®, rinsed with EtOAc (50 mL), the filtrate was washed with water and brine, dried (Na₂SO₄) and evaporated. Flash chromatography (silica, 0-50% EtOAc in iso-hexane) afforded the product (309 mg, 31% yield).

[M−^{t}Bu+H]⁺=509.1, 511.1

¹H NMR (DMSO-d6, 400 MHZ) δ 1.18 (3H, dd, J=7.1, 1.8 Hz), 1.33 (3H, t, J=7.1 Hz), 1.42 (9H, s), 2.93 (1H, p, J=5.7 Hz), 3.19 (2H, t, J=6.6 Hz), 3.40-3.54 (2H, m), 4.06 (2H, t, J=6.6 Hz), 4.35 (3H, q, J=7.1 Hz), 4.59-4.70 (1H, m), 7.15-7.21 (2H, m), 7.26 (1H, d, J=8.3 Hz), 7.49-7.57 (2H, m), 7.59 (1H, dt, J=7.4, 1.8 Hz), 7.73 (1H, t, J=2.0 Hz)

### 1-(3-Chlorophenyl)-7-oxo-6-[(4R)-2-tert-butoxycarbonyl-4-methyl-3,4-dihydro-1H-isoquinolin-7-yl]-4,5-dihydropyrazolo[3,4-c]pyridine-3-carboxylic acid

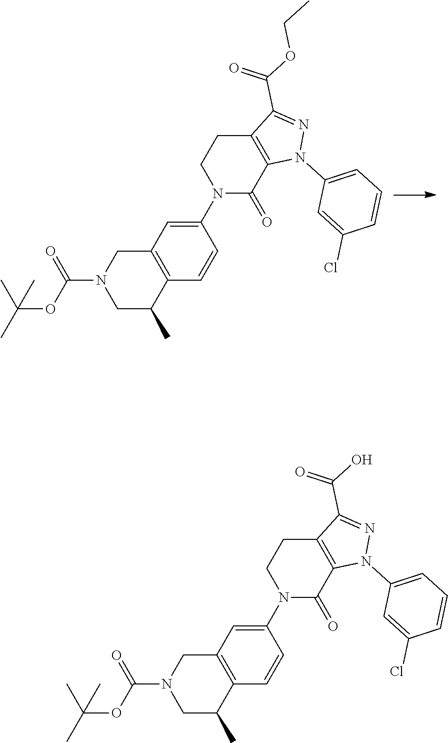 [SMILES: CCOC(=O)c1nn(-c2cccc(Cl)c2)c2c1CCN(c1ccc3c(c1)CN(C(=O)OC(C)(C)C)C[C@@H]3C)C2=O ; C[C@H]1CN(C(=O)OC(C)(C)C)Cc2cc(N3CCc4c(C(=O)O)nn(-c5cccc(Cl)c5)c4C3=O)ccc21]

To a solution of tert-butyl (4R)-7-[1-(3-chlorophenyl)-3-ethoxycarbonyl-7-oxo-4,5-dihydropyrazolo[3,4-c]pyridin-6-yl]-4-methyl-3,4-dihydro-1H-isoquinoline-2-carboxylate (305 mg, 0.54 mmol) in a mixture of THF (4 mL) and water (2 mL) was added LiOH. H₂O (45 mg, 1.08 mmol) and the mixture was stirred at rt for 18 h. The THF was removed and the remaining aqueous solution was acidified to pH=2-3 using 1M HCl (aq). The acidified aqueous phase was extracted with EtOAc and the combined organic extracts were dried (Na₂SO₄) and evaporated to afford the product (299 mg, 100% yield).

[M−^{t}Bu+H]⁺=481.2, 483.2

¹H NMR (DMSO-d6, 400 MHZ) δ 1.15-1.22 (3H, m), 1.42 (9H, s), 2.93 (1H, p, J=5.7 Hz), 3.17 (2H, t, J=6.6 Hz), 3.40-3.54 (2H, m), 4.03-4.10 (2H, m), 4.25-4.48 (1H, m), 4.60-4.71 (1H, m), 7.14-7.21 (2H, m), 7.26 (1H, d, J=8.3 Hz), 7.47-7.56 (2H, m), 7.58 (1H, dt, J=7.0, 2.0 Hz), 7.72-7.74 (1H, m), 13.26 (1H, s br)

### tert-Butyl (4R)-7-[1-(3-chlorophenyl)-3-[(4-methyl-3-pyridyl)methylcarbamoyl]-7-oxo-4,5-dihydropyrazolo[3,4-c]pyridin-6-yl]-4-methyl-3,4-dihydro-1H-isoquinoline-2-carboxylate

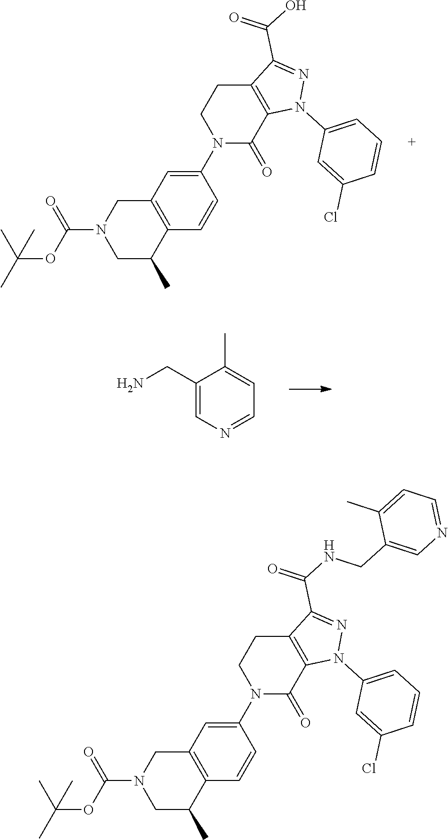

To a solution of 1-(3-chlorophenyl)-7-oxo-6-[(4R)-2-tert-butoxycarbonyl-4-methyl-3,4-dihydro-1H-isoquinolin-7-yl]-4,5-dihydropyrazolo[3,4-c]pyridine-3-carboxylic acid (145 mg, 0.27 mmol) in MeCN (3 mL) were added (4-methylpyridin-3-yl) methanamine (66 mg, 0.54 mmol), 1-methylimidazole (86 μL, 1.08 mmol) and TCFH (114 mg, 0.41 mmol) and the mixture stirred at rt for 20 h. The mixture was diluted with EtOAc (75 mL) and washed with sat. NaHCO₃(aq) and brine, dried (Na₂SO₄) and evaporated. Flash chromatography (silica, 0-100% EtOAc in iso-hexane) afforded the product (142 mg, 82% yield).

[MH]⁺=641.4, 643.4

¹H NMR (DMSO-d6, 400 MHZ) δ 1.18 (3H, d, J=6.8 Hz), 1.42 (9H, s), 2.36 (3H, s), 2.93 (1H, p, J=5.5 Hz), 3.20 (2H, t, J=6.6 Hz), 3.41-3.56 (2H, m), 4.04 (2H, t, J=6.7 Hz), 4.26-4.45 (1H, m), 4.48 (2H, d, J=6.0 Hz), 4.60-4.71 (1H, m), 7.13-7.22 (3H, m), 7.25 (1H, d, J=8.3 Hz), 7.46-7.57 (2H, m), 7.63 (1H, dt, J=6.9, 2.0 Hz), 7.75-7.80 (1H, m), 8.32 (1H, d, J=4.9 Hz), 8.44 (1H, s), 8.99 (1H, t, J=6.0 Hz)

### 1-(3-Chlorophenyl)-N-[(4-methyl-3-pyridyl)methyl]-7-oxo-6-[(4R)-4-methyl-1,2,3,4-tetrahydroisoquinolin-7-yl]-4,5-dihydropyrazolo[3,4-c]pyridine-3-carboxamide hydrochloride

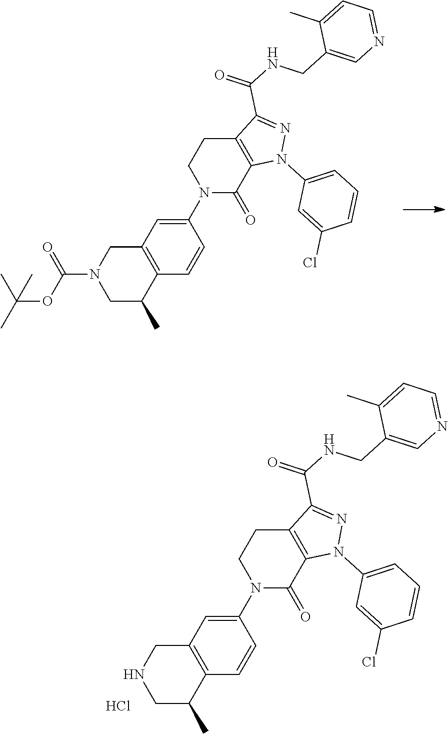 [SMILES: Cc1ccncc1CNC(=O)c1nn(-c2cccc(Cl)c2)c2c1CCN(c1ccc3c(c1)CN(C(=O)OC(C)(C)C)C[C@@H]3C)C2=O ; Cl ; Cc1ccncc1CNC(=O)c1nn(-c2cccc(Cl)c2)c2c1CCN(c1ccc3c(c1)CNC[C@@H]3C)C2=O]

To a solution of tert-butyl (4R)-7-[1-(3-chlorophenyl)-3-[(4-methyl-3-pyridyl)methylcarbamoyl]-7-oxo-4,5-dihydropyrazolo[3,4-c]pyridin-6-yl]-4-methyl-3,4-dihydro-1H-isoquinoline-2-carboxylate (140 mg, 0.22 mmol) in 1,4-dioxan (1.5 mL) was added 4M HCl in 1,4-dioxane (1.5 mL). After 72 h at rt the solvent was removed to afford the product (135 mg, 100% yield).

[M+H]⁺=541.2, 543.2

¹H NMR (DMSO-d6, 400 MHZ) δ 1.32 (3H, d, J=7.0 Hz), 2.61 (3H, s), 3.03 (1H, t, J=9.8 Hz), 3.20 (2H, t, J=6.6 Hz), 4.04 (2H, t, J=6.6 Hz), 4.21-4.26 (2H, m), 4.60 (2H, d, J=5.8 Hz), 7.23 (1H, d, J=2.2 Hz), 7.30 (1H, dd, J=8.4, 2.2 Hz), 7.41 (1H, d, J=8.5 Hz), 7.48-7.59 (2H, m), 7.63 (1H, dt, J=7.2, 1.9 Hz), 7.75-7.80 (1H, m), 7.82 (1H, d, J=5.7 Hz), 8.64-8.70 (2H, m), 9.17 (1H, t, J=5.9 Hz), 9.26 (1H, s br), 9.39 (1H, s br)

### (R)-1-(3-Chlorophenyl)-6-(2-(2-hydroxyethyl)-4-methyl-1,2,3,4-tetrahydroisoquinolin-7-yl)-N-((4-methylpyridin-3-yl)methyl)-7-oxo-4,5,6,7-tetrahydro-1H-pyrazolo[3,4-c]pyridine-3-carboxamide

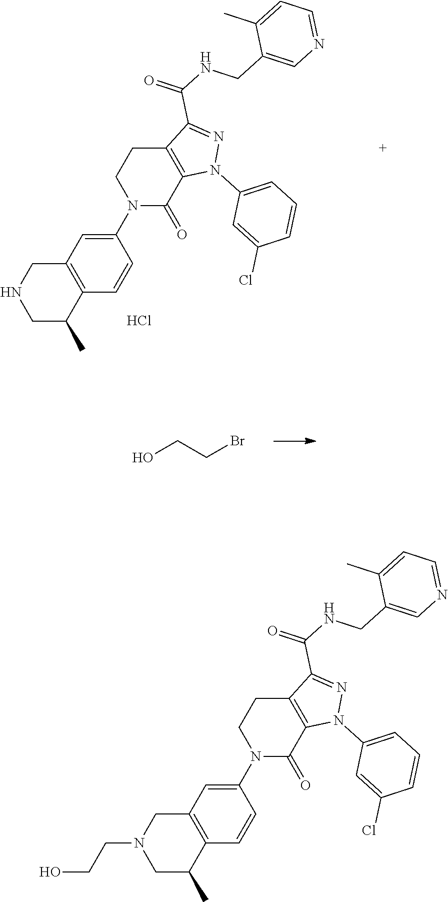

To a solution of 1-(3-chlorophenyl)-N-[(4-methyl-3-pyridyl)methyl]-7-oxo-6-[(4R)-4-methyl-1,2,3,4-tetrahydroisoquinolin-7-yl]-4,5-dihydropyrazolo[3,4-c]pyridine-3-carboxamide hydrochloride (70 mg, 0.12 mmol) in dry DMF (1.5 mL) were added TEA (51 μL, 0.36 mmol) and 2-bromoethanol (17 μL, 0.24 mmol) and the mixture stirred at rt for 20 h. The mixture was diluted with EtOAc (50 mL) and washed with a sat. NaHCO₃(aq) and brine, dried (Na₂SO₄) and evaporated. Flash chromatography (silica, 0-10% MeOH in DCM) followed by ion-exchange using SCX-2 column, washing with MeOH (10 mL) and eluting with 3M NH₃ in MeOH (10 mL) afforded the product (32 mg, 46% yield).

[M+H]⁺=585.3, 587.3

¹H NMR (DMSO-d6, 400 MHZ) δ 1.23 (3H, d, J=6.9 Hz), 2.36 (3H, s), 2.37-2.41 (1H, m), 2.52-2.57 (2H, m), 2.74-2.82 (1H, m), 2.89-2.97 (1H, m), 3.19 (2H, t, J=6.6 Hz), 3.54-3.61 (4H, m), 4.02 (2H, t, J=6.6 Hz), 4.44 (1H, s br), 4.47 (2H, d, J=6.0 Hz), 7.02 (1H, d, J=2.2 Hz), 7.13 (1H, dd, J=8.2, 2.2 Hz), 7.19 (1H, d, J=5.0 Hz), 7.24 (1H, d, J=8.3 Hz), 7.46-7.56 (2H, m), 7.62 (1H, dt, J=7.0, 2.0 Hz), 7.75-7.80 (1H, m), 8.32 (1H, d, J=4.9 Hz), 8.43 (1H, s), 8.99 (1H, t, J=6.0 Hz)

### Example number 1159

### 1-(3-Chlorophenyl)-6-(2-methyl-1-oxo-1,2,3,4-tetrahydroisoquinolin-7-yl)-N-((4-methylpyridin-3-yl)methyl)-7-oxo-4,5,6,7-tetrahydro-1H-pyrazolo[3,4-c]pyridine-3-carboxamide

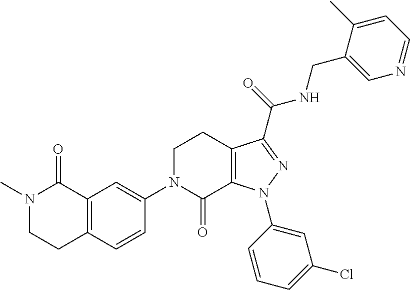 [SMILES: Cc1ccncc1CNC(=O)c1nn(-c2cccc(Cl)c2)c2c1CCN(c1ccc3c(c1)C(=O)N(C)CC3)C2=O]

### Ethyl 1-(3-chlorophenyl)-6-(2-methyl-1-oxo-3,4-dihydroisoquinolin-7-yl)-7-oxo-4,5-dihydropyrazolo[3,4-c]pyridine-3-carboxylate

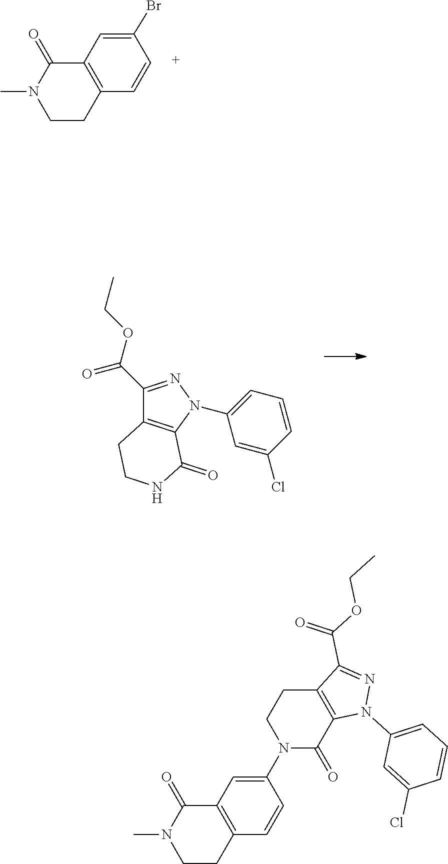 [SMILES: CN1CCc2ccc(Br)cc2C1=O ; CCOC(=O)c1nn(-c2cccc(Cl)c2)c2c1CCNC2=O ; CCOC(=O)c1nn(-c2cccc(Cl)c2)c2c1CCN(c1ccc3c(c1)C(=O)N(C)CC3)C2=O]

Cu(I)I (30 mg, 0.16 mmol) was added to ethyl 1-(3-chlorophenyl)-7-oxo-5,6-dihydro-4H-pyrazolo[3,4-c]pyridine-3-carboxylate (250 mg, 0.78 mmol), 7-bromo-2-methyl-3,4-dihydroisoquinolin-1-one (225 mg, 0.94 mmol), (1S,2S)—N,N′-dimethyl-1,2-cyclohexane-diamine (44 mg, 0.31 mmol), K₃PO₄ (415 mg, 1.95 mmol) and 4 Å molecular sieves (10% w/w) in anhydrous 1,4-dioxane (10 mL). The sealed vessel was stirred at 100° C. for 20 h. The mixture was allowed to cool to rt, filtered through Celite® and the residue was washed with EtOAc (5×10 mL). The combined filtrates were washed with water (20 mL), brine (20 mL), dried (Na₂SO₄), evaporated and purified by flash chromatography (silica, 0-100% EtOAc in iso-hexane), to afford the product (60 mg, 16% yield).

[M+H]⁺=479.2, 481.2

¹H NMR (DMSO-d6, 400 MHZ) δ 1.33 (3H, t, J=7.1 Hz), 2.98 (2H, t, J=6.6 Hz), 3.03 (3H, s), 3.21 (2H, t, J=6.6 Hz), 3.55 (2H, t, J=6.6 Hz), 4.09 (2H, t, J=6.6 Hz), 4.35 (2H, q, J=6.9 Hz), 7.32 (1H, d, J=8.1 Hz), 7.43 -7.47 (1H, m), 7.49-7.57 (2H, m), 7.58-7.62 (1H, m), 7.72-7.79 (1H, m), 7.79-7.85 (1H, m)

### 1-(3-Chlorophenyl)-6-(2-methyl-1-oxo-3,4-dihydroisoquinolin-7-yl)-7-oxo-4,5-dihydropyrazolo[3,4-c]pyridine-3-carboxylic acid

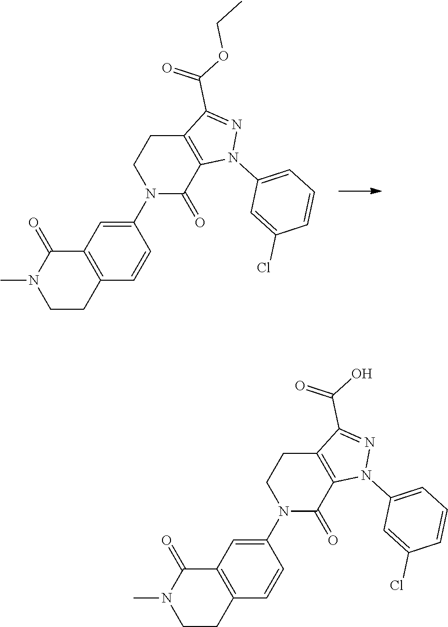 [SMILES: CCOC(=O)c1nn(-c2cccc(Cl)c2)c2c1CCN(c1ccc3c(c1)C(=O)N(C)CC3)C2=O ; CN1CCc2ccc(N3CCc4c(C(=O)O)nn(-c5cccc(Cl)c5)c4C3=O)cc2C1=O]

To a solution of ethyl 1-(3-chlorophenyl)-6-(2-methyl-1-oxo-3,4-dihydroisoquinolin-7-yl)-7-oxo-4,5-dihydropyrazolo[3,4-c]pyridine-3-carboxylate (58 mg, 0.12 mmol) in a mixture of THF (1 mL) and water (0.5 mL) was added LiOH·H₂O (10 mg, 0.24 mmol). The mixture was stirred at rt for 72 h. The THF was removed, the pH of the residue was adjusted to pH7 with 2M HCl (aq) and extracted with CHCl₃/IPA (4:1, 3×50 mL). The combined organic extracts were dried (Na₂SO₄) and evaporated to afford the product (58 mg, 100% yield).

[M+H]⁺=451.1

### 1-(3-Chlorophenyl)-6-(2-methyl-1-oxo-1,2,3,4-tetrahydroisoquinolin-7-yl)-N-((4-methylpyridin-3-yl)methyl)-7-oxo-4,5,6,7-tetrahydro-1H-pyrazolo[3,4-c]pyridine-3-carboxamide

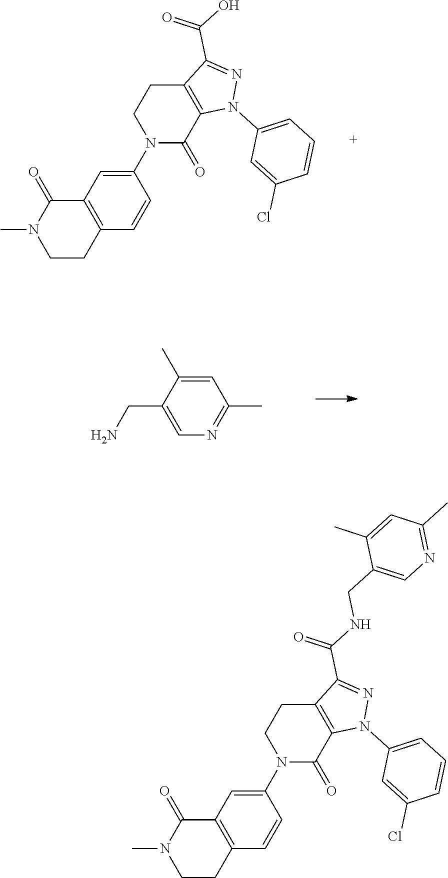

To a solution of 1-(3-chlorophenyl)-6-(2-methyl-1-oxo-3,4-dihydroisoquinolin-7-yl)-7-oxo-4,5-dihydropyrazolo[3,4-c]pyridine-3-carboxylic acid (57 mg, 0.13 mmol), in dry MeCN (3 mL) under N₂ (g), were added (4-methylpyridin-3-yl) methanamine (31 mg, 0.25 mmol), 1-methylimidazole (10 μL, 0.13 mmol) and TCFH (53 mg, 0.19 mmol). The mixture was stirred at rt for 18 h. This solution was diluted with EtOAc (75 mL), washed with sat. NaHCO₃(aq) and brine, dried (Na₂SO₄), evaporated and purified by flash chromatography (silica, 40-100% (EtOAc: MeOH (9:1) in DCM) and then 0-5% MeOH in DCM), to afford the product (56 mg, 92% yield).

[M+H]⁺=555.3, 557.2

¹H NMR (DMSO-d6, 400 MHZ) δ 2.36 (3H, s), 2.97 (2H, t, J=6.6 Hz), 3.03 (3H, s), 3.22 (2H, t, J=6.6 Hz), 3.55 (2H, t, J=6.7 Hz), 4.06 (2H, t, J=6.6 Hz), 4.48 (2H, d, J=6.0 Hz), 7.19 (1H, d, J=5.0 Hz), 7.31 (1H, d, J=8.2 Hz), 7.45 (1H, dd, J=8.1, 2.3 Hz), 7.47-7.54 (2H, m), 7.64 (1H, dt, J=6.6, 2.1 Hz), 7.76-7.85 (2H, m), 8.32 (1H, d, J=4.9 Hz), 8.44 (1H, s), 8.99 (1H, t, J=6.0 Hz)

### Example Number 1169

### 1-(3-Chlorophenyl)-N-(cyclopropylmethyl)-6-(2-ethyl-5-(methoxymethyl)-1,2,3,4-tetrahydroisoquinolin-7-yl)-7-oxo-4,5,6,7-tetrahydro-1H-pyrazolo[3,4-c]pyridine-3-carboxamide

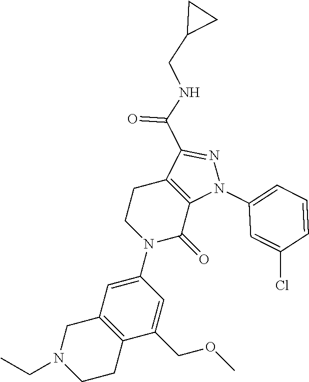 [SMILES: CCN1CCc2c(COC)cc(N3CCc4c(C(=O)NCC5CC5)nn(-c5cccc(Cl)c5)c4C3=O)cc2C1]

### tert-Butyl 7-bromo-5-formyl-3,4-dihydro-1H-isoquinoline-2-carboxylate

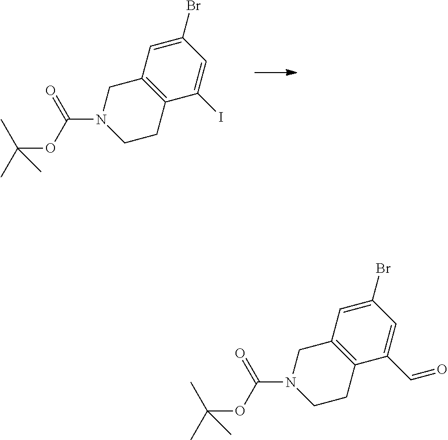 [SMILES: CC(C)(C)OC(=O)N1CCc2c(I)cc(Br)cc2C1 ; CC(C)(C)OC(=O)N1CCc2c(C=O)cc(Br)cc2C1]

To a solution of tert-butyl 7-bromo-5-iodo-3,4-dihydro-1H-isoquinoline-2-carboxylate (1.00 g, 2.28 mmol) in dry THF (20 mL) at −78° C. (acetone/dry ice bath), was added isopropylmagnesium chloride lithium chloride complex (Turbo-Grignard), 1.3 M solution in THF (1.4 mL, 1.8 mmol) and the mixture was stirred for 30 min. DMF (0.71 mL, 9.1 mmol) was added dropwise and the reaction was stirred for 20 h allowing it to warm to rt. The solution was quenched with sat. NH₄CI (aq)(10 ml) and diluted with EtOAc (100 mL). The organic layer was washed with sat. NH₄CI (aq)(25 ml) and brine (25 ml), dried (Na₂SO₄), filtered, evaporated and purified by flash chromatography (silica, 0-30% EtOAc in iso-hexane) to provide the product (159 mg, 20% yield).

[M−Boc+H]⁺=240.0/242.

¹H NMR (DMSO-d6, 400 MHz) 8 1.42 (9H, s), 3.16 (2H, t, J=6.1 Hz), 3.56 (2H, t, J=5.9 Hz), 4.58 (2H, s), 7.77 (1H, d, J=2.3 Hz), 7.88 (1H, d, J=2.2 Hz), 10.15 (1H, s)

### tert-Butyl 7-bromo-5-(methoxymethyl)-3,4-dihydro-1H-isoquinoline-2-carboxylate

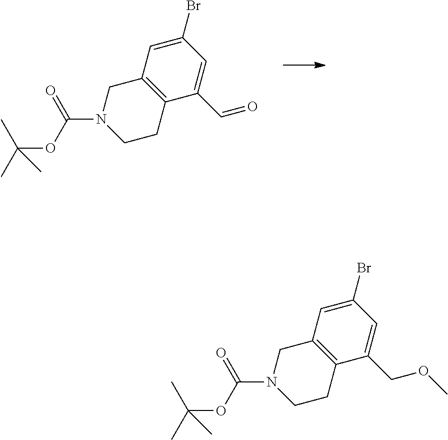 [SMILES: CC(C)(C)OC(=O)N1CCc2c(C=O)cc(Br)cc2C1 ; COCc1cc(Br)cc2c1CCN(C(=O)OC(C)(C)C)C2]

To a solution of tert-butyl 7-bromo-5-formyl-3,4-dihydro-1H-isoquinoline-2-carboxylate (155 mg, 0.456 mmol) in dry DCM (5 mL) at 0° C. were added MeOH (0.092 mL, 2.28 mmol), triethylsilane (0.22 mL, 1.4 mmol) and finally trimethylsilyl trifluoromethanesulphonate (0.16 mL, 0.91 mmol), and the mixture was stirred for 3h, allowing it to warm up to rt. The reaction was quenched with sat. NaHCO₃(aq)(1 mL) and the mixture was diluted with DCM (50 mL) and washed with sat. NaHCO₃(aq)(15 mL) and brine (15 mL), dried (Na₂SO₄), filtered and evaporated. The residue was dissolved in DCM (5 mL), followed by the addition of TEA (0.16 mL, 1.14 mmol) and Boc₂O (149 mg, 0.683 mmol), and the mixture was allowed to stir for 20 h at rt. The reaction was diluted with DCM (50 mL) and washed with sat. NaHCO₃(aq)(15 mL) and brine (15 mL), dried (Na₂SO₄), filtered, evaporated and purified by flash chromatography (silica, 0-30% EtOAc in iso-hexane), to provide the product (129 mg, 80% yield).

[M−Boc+H]⁺=256.1/258.1

¹H NMR (DMSO-d6, 400 MHZ) δ 1.42 (9H, s), 2.66 (2H, t, J=6.0 Hz), 3.31 (3H, s), 3.54 (2H, t, J=5.9 Hz), 4.39 (2H, s), 4.50 (2H, s), 7.36 (2H, s)

### tert-Butyl 7-[1-(3-chlorophenyl)-3-ethoxycarbonyl-7-oxo-4,5-dihydropyrazolo[3,4-c]pyridin-6-yl]-5-(methoxymethyl)-3,4-dihydro-1H-isoquinoline-2-carboxylate

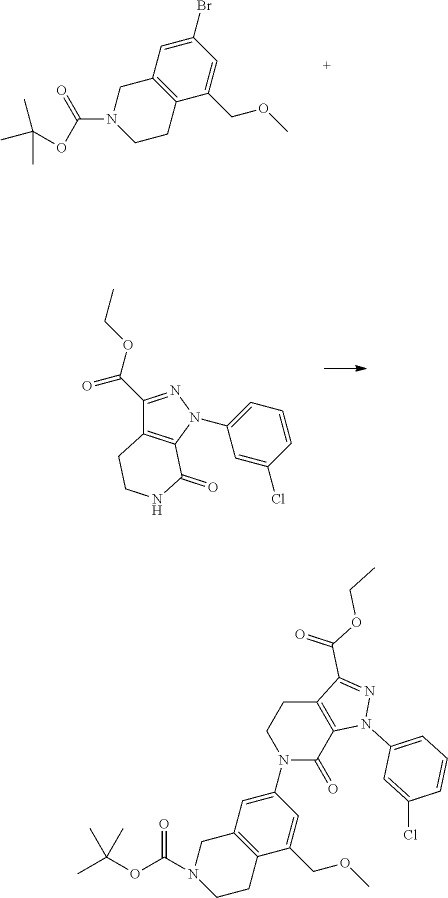

A solution of ethyl 1-(3-chlorophenyl)-7-oxo-5,6-dihydro-4H-pyrazolo[3,4-c]pyridine-3-carboxylate (161 mg, 0.503 mmol) and tert-butyl 7-bromo-5-(methoxymethyl)-3,4-dihydro-1H-isoquinoline-2-carboxylate (128 mg, 0.359 mmol) in dry 1,4-dioxane (3.5 mL) was bubbled with N₂ (g) for 5 min. After this time, and maintaining the bubbling, were sequentially added K₃PO₄ (191 mg, 0.898 mmol), (1S,2S)—N,N′-dimethyl-1,2-cyclohexanediamine (20 mg, 0.14 mmol) and, finally, Cu(I)I (14 mg, 0.072 mmol). The system was closed and stirred at 100° C. for 18 h. The solution was then filtered through Celite®, rinsed with EtOAc (50 mL), and the filtrate washed with water (20 mL) and brine (2×20 mL), dried (Na₂SO₄), filtered, evaporated and purified by flash chromatography (silica, 1-50% EtOAc in iso-hexane) to provide the product (29 mg, 13% yield).

[M−tBu+H]⁺=539.1/541.1

¹H NMR (DMSO-d6, 400 MHZ) δ 1.33 (3H, t, J=7.1 Hz), 1.42 (9H, s), 2.71 (2H, t, J=6.0 Hz), 3.19 (2H, t, J=6.6 Hz), 3.31 (3H, s), 3.56 (2H, t, J=5.9 Hz), 4.06 (2H, t, J=6.6 Hz), 4.35 (2H, q, J=7.1 Hz), 4.39 (2H, s), 4.50 (2H, s br), 7.12 (1H, d, J=2.2 Hz), 7.19 (1H, d, J=2.2 Hz), 7.49-7.57 (2H, m), 7.58 (1H, dt, J=7.4, 1.9 Hz), 7.73 (1H, t, J=2.0 Hz)

### 6-[2-tert-Butoxycarbonyl-5-(methoxymethyl)-3,4-dihydro-1H-isoquinolin-7-yl]-1-(3-chlorophenyl)-7-oxo-4,5-dihydropyrazolo[3,4-c]pyridine-3-carboxylic acid

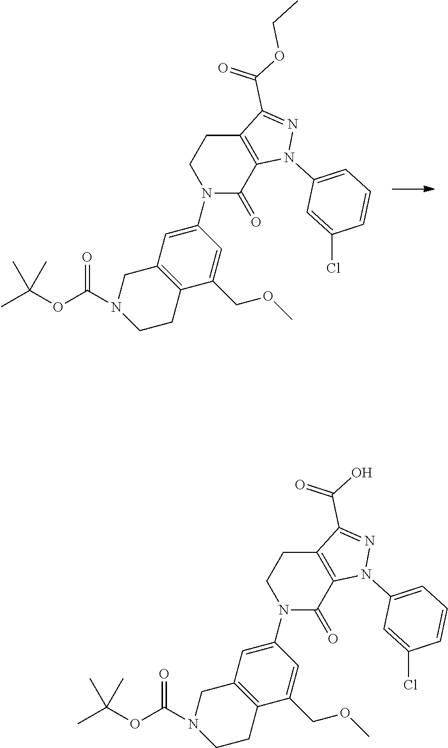 [SMILES: CCOC(=O)c1nn(-c2cccc(Cl)c2)c2c1CCN(c1cc(COC)c3c(c1)CN(C(=O)OC(C)(C)C)CC3)C2=O ; COCc1cc(N2CCc3c(C(=O)O)nn(-c4cccc(Cl)c4)c3C2=O)cc2c1CCN(C(=O)OC(C)(C)C)C2]

To a solution of tert-butyl 7-[1-(3-chlorophenyl)-3-ethoxycarbonyl-7-oxo-4,5-dihydropyrazolo[3,4-c]pyridin-6-yl]-5-(methoxymethyl)-3,4-dihydro-1H-isoquinoline-2-carboxylate (28 mg, 0.047 mmol) in a mixture of THF (1 mL) and water (0.5 mL) was added LiOH·H₂O (3.9 mg, 0.094 mmol) and the mixture stirred at rt for 2 h. The mixture was neutralised with the addition of 1M HCl (aq)(0.1 mL) and evaporated to provide the product (29 mg, 100% yield).

[M−tBu+H]⁺=511.1/513.1

### tert-Butyl 7-[1-(3-chlorophenyl)-3-(cyclopropylmethylcarbamoyl)-7-oxo-4,5-dihydropyrazolo[3,4-c]pyridin-6-yl]-5-(methoxymethyl)-3,4-dihydro-1H-isoquinoline-2-carboxylate

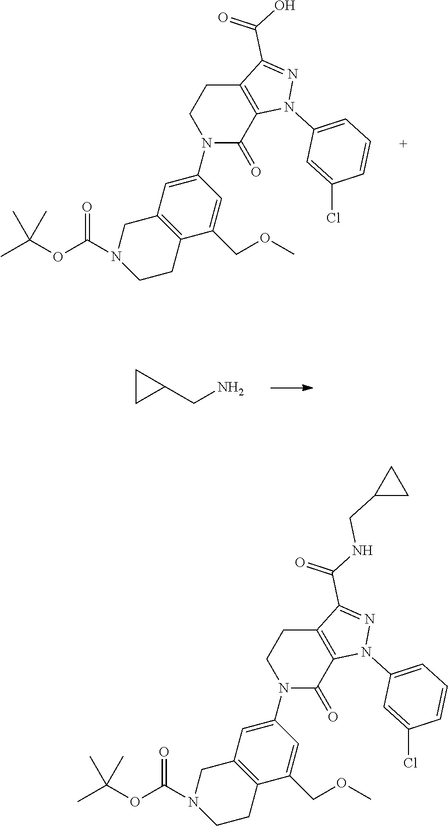 [SMILES: COCc1cc(N2CCc3c(C(=O)O)nn(-c4cccc(Cl)c4)c3C2=O)cc2c1CCN(C(=O)OC(C)(C)C)C2 ; NCC1CC1 ; COCc1cc(N2CCc3c(C(=O)NCC4CC4)nn(-c4cccc(Cl)c4)c3C2=O)cc2c1CCN(C(=O)OC(C)(C)C)C2]

To a solution of 6-[2-tert-butoxycarbonyl-5-(methoxymethyl)-3,4-dihydro-1H-isoquinolin-7-yl]-1-(3-chlorophenyl)-7-oxo-4,5-dihydropyrazolo[3,4-c]pyridine-3-carboxylic acid (27 mg, 0.048 mmol) in dry MeCN (1.5 mL) were added aminomethylcyclopropane (0.021 mL, 0.24 mmol), 1-methylimidazole (0.034 mL, 0.43 mmol) and TCFH (27 mg, 0.095 mmol) and the mixture stirred for 20 h at rt. The solution was diluted with EtOAc (50 mL), washed with sat. NaHCO₃(aq)(15 mL) and brine (15 mL), dried (Na₂SO₄), filtered, evaporated and purified by flash chromatography (silica, 2-80% EtOAc in iso-hexane), to provide the product (23 mg, 77% yield).

[M−tBu+H]⁺=564.2/566.2

¹H NMR (DMSO-d6, 400 MHZ) δ 0.21-0.27 (2H, m), 0.39-0.45 (2H, m), 1.00-1.08 (1H, m), 1.42 (9H, s), 2.70 (2H, t, J=6.2 Hz), 3.12 (2H, t, J=6.5 Hz), 3.20 (2H, t, J=6.6 Hz), 3.32 (3H, s), 3.53-3.59 (2H, m), 4.04 (2H, t, J=6.6 Hz), 4.39 (2H, s), 4.50 (2H, s br), 7.12 (1H, s), 7.19 (1H, s), 7.48-7.56 (2H, m), 7.62 (1H, dd, J=8.1, 1.8 Hz), 7.75-7.79 (1H, m), 8.50 (1H, t, J=6.0 Hz

### 1-(3-Chlorophenyl)-N-(cyclopropylmethyl)-6-[5-(methoxymethyl)-1,2,3,4-tetrahydroisoquinolin-7-yl]-7-oxo-4,5-dihydropyrazolo[3,4-c]pyridine-3-carboxamide hydrochloride

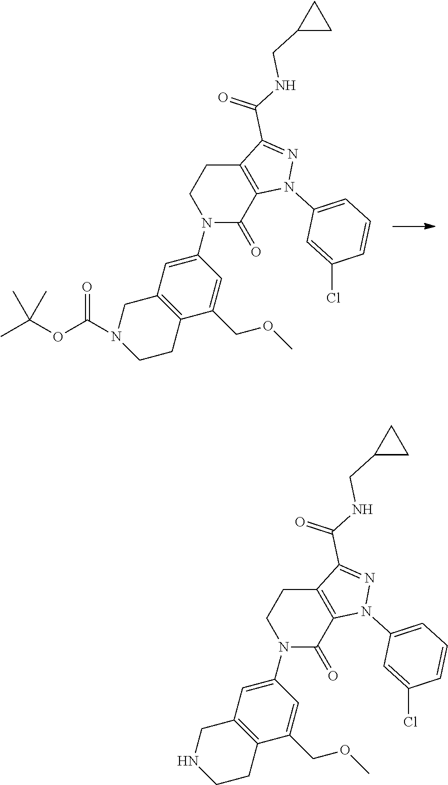 [SMILES: COCc1cc(N2CCc3c(C(=O)NCC4CC4)nn(-c4cccc(Cl)c4)c3C2=O)cc2c1CCN(C(=O)OC(C)(C)C)C2 ; COCc1cc(N2CCc3c(C(=O)NCC4CC4)nn(-c4cccc(Cl)c4)c3C2=O)cc2c1CCNC2]

To a solution of tert-butyl 7-[1-(3-chlorophenyl)-3-(cyclopropylmethylcarbamoyl)-7-oxo-4,5-dihydropyrazolo[3,4-c]pyridin-6-yl]-5-(methoxymethyl)-3,4-dihydro-1H-isoquinoline-2-carboxylate (22 mg, 0.036 mmol) in 1,4-dioxane (0.5 mL) was added HCl (4M in 1,4-dioxane)(0.52 mL, 2.1 mmol) and the mixture stirred for 6 h at rt. The solvent was evaporated to provide the product (21 mg, 100% yield).

[M−HCl+H]⁺=520.2/522.2

### 1-(3-Chlorophenyl)-N-(cyclopropylmethyl)-6-(2-ethyl-5-(methoxymethyl)-1,2,3,4-tetrahydroisoquinolin-7-yl)-7-oxo-4,5,6,7-tetrahydro-1H-pyrazolo[3,4-c]pyridine-3-carboxamide

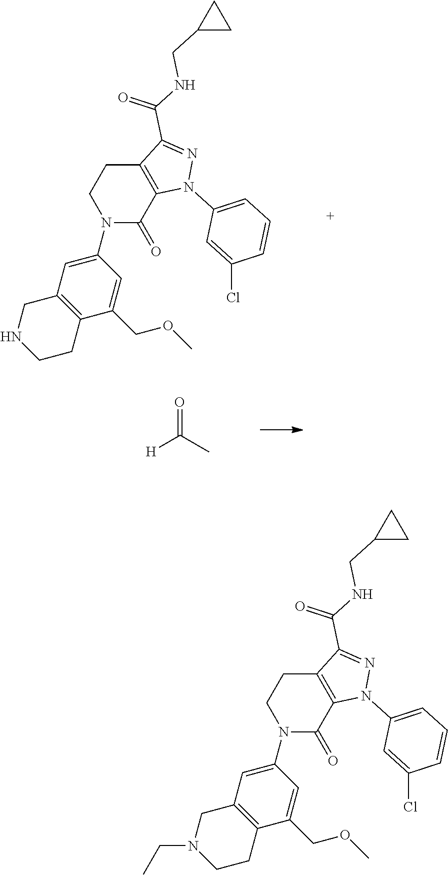

To a solution of 1-(3-chlorophenyl)-N-(cyclopropylmethyl)-6-[5-(methoxymethyl)-1,2,3,4-tetrahydroisoquinolin-7-yl]-7-oxo-4,5-dihydropyrazolo[3,4-c]pyridine-3-carboxamide hydrochloride (21 mg, 0.038 mmol) in a mixture of DCM (2 mL) and MeOH (0.4 mL) were added TEA (0.021 mL, 0.15 mmol) and acetaldehyde (50% in EtOH)(0.013 mL, 0.11 mmol) and the mixture stirred at rt for 1 h. STAB (16 mg, 0.076 mmol) was added portion-wise and the mixture stirred for 20 h. The solution was diluted with DCM (50 mL) and washed with sat. NaHCO₃(aq)(15 mL) and brine (15 mL), dried (Na₂SO₄), filtered, evaporated, purified by preparative HPLC (C18, 45-70% [10 mM NH₄CO₃ in MeCN+0.1% NH₃]) and lyophilised to provide the product (8.0 mg, 38% yield).

[M+H]⁺=548.3/550.3

¹H NMR (DMSO-d6, 400 MHZ) δ 0.21-0.27 (2H, m), 0.39-0.45 (2H, m), 1.00-1.05 (1H, m), 1.08 (3H, t, J=7.2 Hz), 2.46 (2H, q, J=7.2 Hz), 2.62-2.67 (2H, m), 2.71 (2H, t, J=5.8 Hz), 3.12 (2H, t, J=6.4 Hz), 3.19 (2H, t, J=6.6 Hz), 3.31 (3H, s), 3.51 (2H, s), 4.02 (2H, t, J=6.6 Hz), 4.36 (2H, s), 7.00 (1H, d, J=2.2 Hz), 7.13 (1H, d, J=2.2 Hz), 7.47-7.56 (2H, m), 7.62 (1H, dt, J=6.8, 2.1 Hz), 7.77 (1H, td, J=1.9, 0.7 Hz), 8.49 (1H, t, J=6.0 Hz)

### Example Number 2002

### 1-(3-Chloro-2-fluorophenyl)-6-(2-(2-hydroxyethyl)-1,2,3,4-tetrahydroisoquinolin-7-yl)-N-((4-methylpyridin-3-yl)methyl)-7-oxo-4,5,6,7-tetrahydro-1H-pyrazolo[3,4-c]pyridine-3-carboxamide

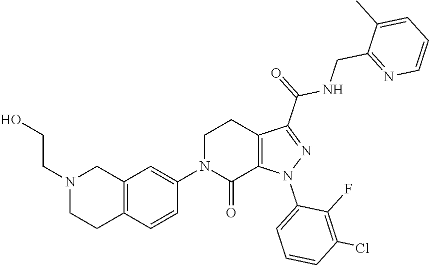 [SMILES: Cc1cccnc1CNC(=O)c1nn(-c2cccc(Cl)c2F)c2c1CCN(c1ccc3c(c1)CN(CCO)CC3)C2=O]

### Ethyl 2-[(3-chloro-2-fluorophenyl) hydrazinylidene] acetate

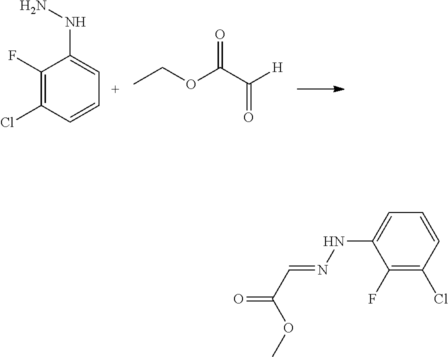

2-Fluoro-3-chlorophenylhydrazine hydrochloride (2000 mg, 10.15 mmol) was suspended in dry THF (50 mL) and TEA (1698 μL, 12.18 mmol) was added. Ethyl 2-oxoacetate (50% solution in toluene)(2280 mg, 11.17 mmol) was added dropwise at 0° C. The mixture was stirred at rt for 18 h. The solid was filtered off and washed with THF (50 mL). The combined filtrates were evaporated and purified by flash chromatography (Silica, 2% MeOH in CHCl₃) to afford the product (2380 mg, 96% yield).

[M+H]⁺=245.0

¹H NMR (CDCl₃, 400 MHZ) δ 1.36 (3H, t, J=7.1 Hz), 4.33 (2H, q, J=7.1 Hz), 6.97 (1H, ddd, J=8.2, 6.7, 1.7 Hz), 7.04 (1H, ddd, J=8.2, 1.5, 0.6 Hz), 7.20 (1H, d, J=1.4 Hz), 7.50-7.59 (1H, m), 8.38 (1H, s)

### Ethyl 2-chloro-2-[(3-chloro-2-fluorophenyl) hydrazinylidene] acetate

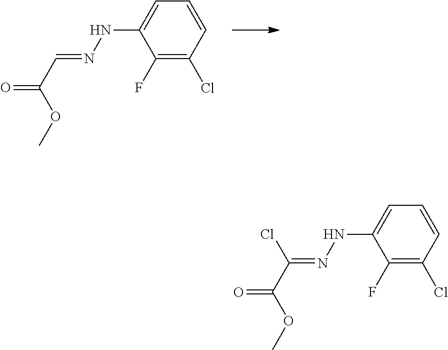 [SMILES: COC(=O)/C=N/Nc1cccc(Cl)c1F ; COC(=O)/C(Cl)=N/Nc1cccc(Cl)c1F]

To a solution of ethyl 2-[(3-chloro-2-fluorophenyl) hydrazinylidene] acetate (2300 mg, 9.4 mmol) in dry THF (20 mL) at 0° C. was added NCS (1506 mg, 11.28 mmol) and the reaction stirred at rt for 18 h. The solvent was removed and the residue was purified by flash chromatography (Silica, 2% MeOH in CHCl₃) to afford the product (1620 mg, 62% yield).

¹H NMR (CDCl₃, 400 MHZ) δ 1.41 (3H, t, J=7.1 Hz), 4.38-4.45 (2H, m), 6.95-7.13 (2H, m), 7.47-7.56 (1H, m), 8.46 (1H, s)

### Ethyl (3aS,7aR)-1-(3-chloro-2-fluorophenyl)-7a-morpholin-4-yl-7-oxo-3a,4,5,6-tetrahydropyrazolo[3,4-c]pyridine-3-carboxylate

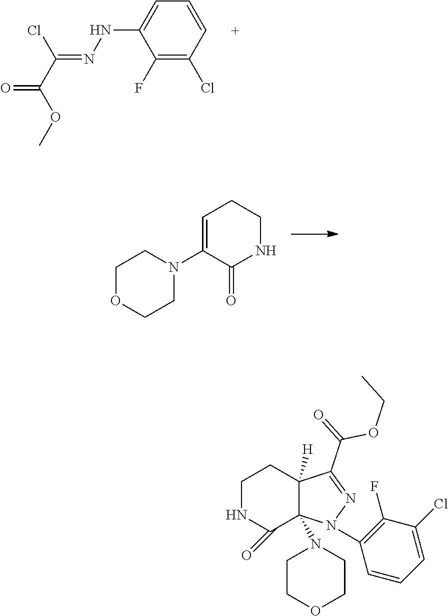 [SMILES: COC(=O)/C(Cl)=N/Nc1cccc(Cl)c1F ; O=C1NCCC=C1N1CCOCC1 ; CCOC(=O)C1=NN(c2cccc(Cl)c2F)[C@@]2(N3CCOCC3)C(=O)NCC[C@@H]12]

Ethyl 2-chloro-2-[(3-chloro-2-fluorophenyl) hydrazinylidene] acetate (1600 mg, 5.73 mmol) was dissolved in EtOAc (50 mL) and 3-(morpholin-4-yl)-1,2,5,6-tetrahydropyridin-2-one (2300 mg, 6.31 mmol) was added at 0° C. The mixture was warmed to rt over 30 min and then heated at reflux for 5 h. The mixture was diluted with EtOAc (50 mL), washed with water (30 mL) and brine (30 mL), dried (Na₂SO₄), evaporated and purified by flash chromatography (Silica, 2% MeOH in CHCl₃) to afford the racemic product (2430 mg, 100% yield).

[M+H]⁺=425.1

### 1-(3-Chloro-2-fluorophenyl)-7-oxo-5,6-dihydro-4H-pyrazolo[3,4-c]pyridine-3-carboxylate

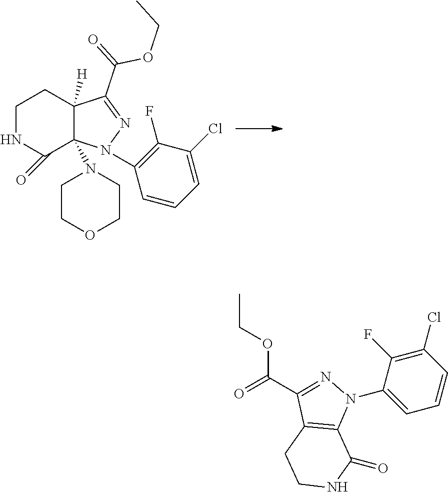 [SMILES: CCOC(=O)C1=NN(c2cccc(Cl)c2F)[C@@]2(N3CCOCC3)C(=O)NCC[C@@H]12 ; CCOC(=O)c1nn(-c2cccc(Cl)c2F)c2c1CCNC2=O]

Ethyl (3aS,7aR)-1-(3-chloro-2-fluorophenyl)-7a-morpholin-4-yl-7-oxo-3a,4,5,6-tetrahydropyrazolo[3,4-c]pyridine-3-carboxylate (760 mg, 1.8 mmol) was dissolved in TFA (10 mL) and DCM (10 mL). After 3 h at rt the solvent was removed and the residue was purified by flash chromatography (Silica, 4% MeOH in CHCl₃) to afford the product (480 mg, 79% yield).

[M+H]⁺=338.0

¹H NMR (CDCl₃, 400 MHZ) δ 1.42 (3H, t, J=7.1 Hz), 3.22 (2H, t, J=7.0 Hz), 3.70 (2H, td, J=7.0, 2.9 Hz), 4.39-4.50 (2H, m), 6.94 (1H, dt, J=5.7, 4.1 Hz), 7.20 (1H, td, J=8.2, 1.6 Hz), 7.45 (1H, ddd, J=8.0, 6.4, 1.6 Hz), 7.50-7.54 (1H, m)

### tert-Butyl 7-[1-(3-chloro-2-fluorophenyl)-3-ethoxycarbonyl-7-oxo-4,5-dihydropyrazolo[3,4-c]pyridin-6-yl]-3,4-dihydro-1H-isoquinoline-2-carboxylate

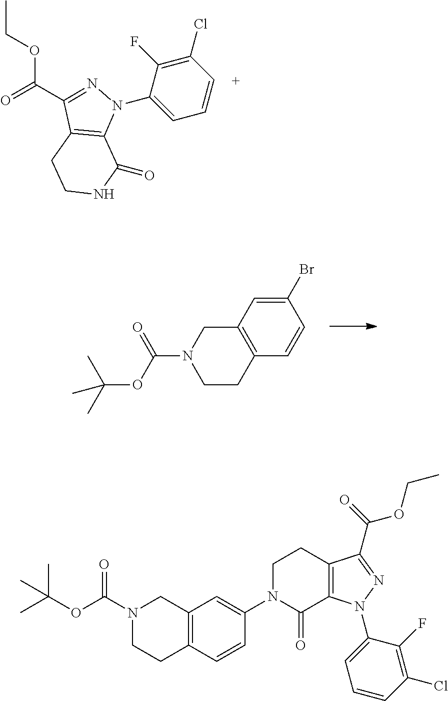 [SMILES: CCOC(=O)c1nn(-c2cccc(Cl)c2F)c2c1CCNC2=O ; CC(C)(C)OC(=O)N1CCc2ccc(Br)cc2C1 ; CCOC(=O)c1nn(-c2cccc(Cl)c2F)c2c1CCN(c1ccc3c(c1)CN(C(=O)OC(C)(C)C)CC3)C2=O]

Ethyl 1-(3-chloro-2-fluorophenyl)-7-oxo-5,6-dihydro-4H-pyrazolo[3,4-c]pyridine-3-carboxylate (400 mg, 1.18 mmol) was dissolved in DMSO (20 mL) and 1,10-phenanthroline (43 mg, 0.24 mmol), Cu(I)I (248 mg, 1.3 mmol), tert-butyl 7-bromo-3,4-dihydroisoquinoline-2 (1H)-carboxylate (407 mg, 1.3 mmol) and K₂CO₃ (327 mg, 2.37 mmol) were added. The mixture was stirred at 130° C. for 18 h after which time it was diluted with EtOAc (70 mL), washed with water (3×30 mL) and brine (30 mL), dried (Na₂SO₄), evaporated and purified by flash chromatography (Silica, 1% MeOH in CHCl₃) to afford the product (160 mg, 24% yield).

[M−tBu+H]⁺=513.2

### Ethyl 1-(3-chloro-2-fluorophenyl)-7-oxo-6-(1,2,3,4-tetrahydroisoquinolin-7-yl)-4,5-dihydropyrazolo[3,4-c]pyridine-3-carboxylate hydrochloride

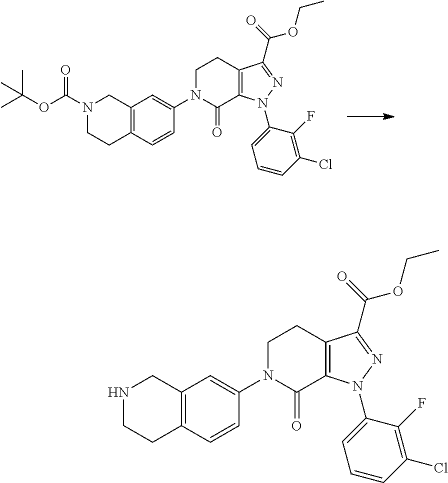 [SMILES: CCOC(=O)c1nn(-c2cccc(Cl)c2F)c2c1CCN(c1ccc3c(c1)CN(C(=O)OC(C)(C)C)CC3)C2=O ; CCOC(=O)c1nn(-c2cccc(Cl)c2F)c2c1CCN(c1ccc3c(c1)CNCC3)C2=O]

tert-Butyl 7-[1-(3-chloro-2-fluorophenyl)-3-ethoxycarbonyl-7-oxo-4,5-dihydropyrazolo[3,4-c]pyridin-6-yl]-3,4-dihydro-1H-isoquinoline-2-carboxylate (160 mg, 0.28 mmol) was dissolved in 4M HCl in 1,4-dioxane (13 mL). After 1 h at rt Et₂O (50 mL) was added and the solid filtered off to afford the product (132 mg, 93% yield).

[M+H]⁺=469.2

### Ethyl 1-(3-chloro-2-fluorophenyl)-6-[2-(2-hydroxyethyl)-3,4-dihydro-1H-isoquinolin-7-yl]-7-oxo-4,5-dihydropyrazolo[3,4-c]pyridine-3-carboxylate

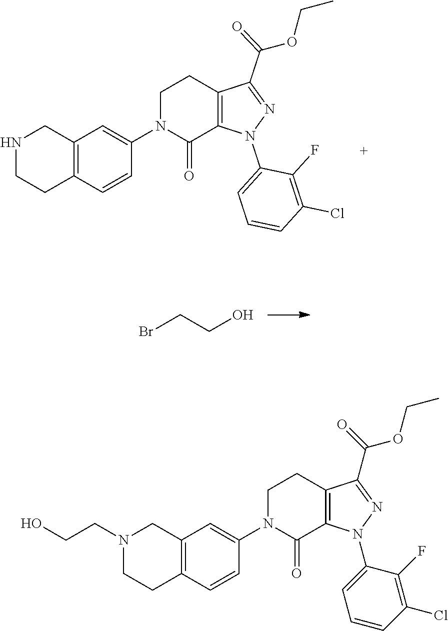

Ethyl 1-(3-chloro-2-fluorophenyl)-7-oxo-6-(1,2,3,4-tetrahydroisoquinolin-7-yl)-4,5-dihydropyrazolo[3,4-c] pyridine-3-carboxylate hydrochloride (142 mg, 0.28 mmol) was dissolved in DMF (15 mL). 2-Bromoethanol (42 mg, 0.34 mmol) and TEA (117 μL, 0.84 mmol) were added. The reaction was stirred at rt for 18 h. The mixture was diluted with EtOAc (100 mL), washed with water (50 mL) and brine (50 mL), dried (Na₂SO₄), filtered, evaporated and purified by flash chromatography (Silica, 6% MeOH in CHCl₃) to afford the product (118 mg, 82% yield).

[M+H]⁺=513.2

### 1-(3-Chloro-2-fluorophenyl)-6-[2-(2-hydroxyethyl)-3,4-dihydro-1H-isoquinolin-7-yl]-7-oxo-4,5-dihydropyrazolo[3,4-c]pyridine-3-carboxylic acid

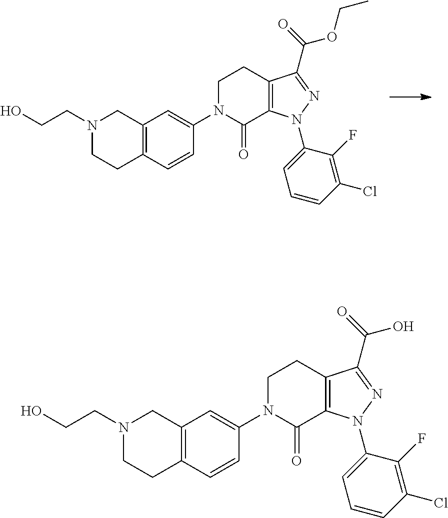 [SMILES: CCOC(=O)c1nn(-c2cccc(Cl)c2F)c2c1CCN(c1ccc3c(c1)CN(CCO)CC3)C2=O ; O=C(O)c1nn(-c2cccc(Cl)c2F)c2c1CCN(c1ccc3c(c1)CN(CCO)CC3)C2=O]

Ethyl 1-(3-chloro-2-fluorophenyl)-6-[2-(2-hydroxyethyl)-3,4-dihydro-1H-isoquinolin-7-yl]-7-oxo-4,5-dihydropyrazolo[3,4-c]pyridine-3-carboxylate (118 mg, 0.23 mmol) was dissolved THF (100 mL) and water (10 mL). LiOH·H₂O (30 mg, 0.69 mmol) was added and the mixture was stirred at rt for 3 days. The THF was removed, the pH of the residue was adjusted to pH7 with 2M HCl (aq) and extracted with CHCl₃/IPA (4:1, 3×50 mL). The combined organic extracts were dried (Na₂SO₄) and evaporated to afford the product (81 mg, 73% yield).

[M+H]⁺=485.1

### 1-(3-Chloro-2-fluorophenyl)-6-(2-(2-hydroxyethyl)-1,2,3,4-tetrahydroisoquinolin-7-yl)-N-((4-methylpyridin-3-yl)methyl)-7-oxo-4,5,6,7-tetrahydro-1H-pyrazolo[3,4-c]pyridine-3-carboxamide

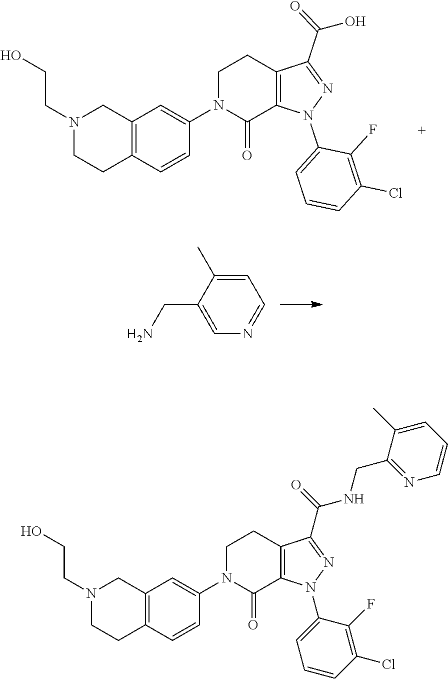

1-(3-Chloro-2-fluorophenyl)-6-[2-(2-hydroxyethyl)-3,4-dihydro-1H-isoquinolin-7-yl]-7-oxo-4,5-dihydropyrazolo[3,4-c]pyridine-3-carboxylic acid (50 mg, 0.10 mmol) and HBTU (29 mg, 0.12 mmol) were dissolved in DCM (30 mL). TEA (43 μL, 0.31 mmol) was added and after 20 min (4-methylpyridin-3-yl)methylamine dihydrochloride (14 mg, 0.11 mmol) was added. The mixture was stirred at rt for 18 h after which time the mixture was diluted with CHCl₃ (100 mL), washed with sat. NaHCO₃(aq)(30 mL), water (30 mL) and brine (30 mL), dried (Na₂SO₄), evaporated, purified by flash chromatography (Silica, 10% MeOH in CHCl₃) and lyophilized to afford the product (28 mg, 46% yield).

[M+H]⁺=589.3

¹H NMR (DMSO-d6, 400 MHZ) δ 2.34 (3H, s), 2.51-2.55 (2H, m), 2.65-2.70 (2H, m), 2.76 (2H, t, J=5.8 Hz), 3.21 (2H, t, J=6.7 Hz), 3.55 (4H, d, J=5.4 Hz), 4.03 (2H, t, J=6.7 Hz), 4.45 (3H, d, J=6.2 Hz), 7.00 (1H, d, J=2.0 Hz), 7.03-7.11 (2H, m), 7.16-7.19 (1H, m), 7.37 (1H, td, J=8.2, 1.4 Hz), 7.65 (1H, ddd, J=8.3, 6.7, 1.6 Hz), 7.76 (1H, ddd, J=8.4, 6.8, 1.6 Hz), 8.29-8.31 (1H, m), 8.41 (1H, s), 9.03 (1H, t, J=6.0 Hz)

### Example Number 3002

### 1-(3-Chlorophenyl)-5-methyl-6-(2-methyl-1,2,3,4-tetrahydroisoquinolin-7-yl)-7-oxo-6,7-dihydro-1H-pyrazolo[4,3-d]pyrimidine-3-carbonitrile

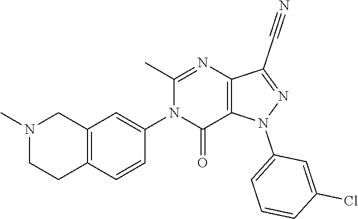 [SMILES: Cc1nc2c(C#N)nn(-c3cccc(Cl)c3)c2c(=O)n1-c1ccc2c(c1)CN(C)CC2]

### 2-[(3-Chlorophenyl) hydrazono]propanedinitrile

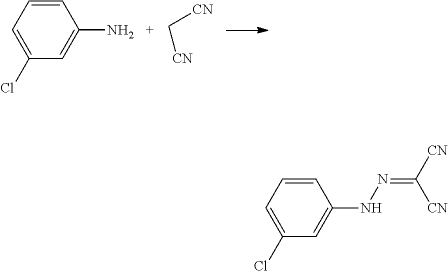 [SMILES: Nc1cccc(Cl)c1 ; N#CCC#N ; N#CC(C#N)=NNc1cccc(Cl)c1]

HCl (12M, 7.4 mL, 86.2 mmol) was added to a suspension of 3-chloroaniline (0.83 mL, 7.8 mmol) in water (40 mL) at 0° C. 1M NaNO₂ (aq)(7.8 mL, 7.8 mmol) was added and the mixture was stirred at 0° C. for 30 min. The mixture was added slowly to a stirring solution of malononitrile (777 mg, 11.8 mmol) and KOAc (23.1 g, 235 mmol) at 0° C. over a period of 30 min. The resulting mixture was stirred at 0° C. for a further 60 min. The solid was filtered off and washed with water (5×10 mL). This solid was taken up in EtOAc (50 mL) and washed with brine, dried (Na₂SO₄) and evaporated to afford the product (1600 mg, 100% yield).

[M+H]⁺=203.1, 205.1

1H (CDCl₃, 400 MHZ) δ 7.14-7.23 (1H, m), 7.20-7.28 (1H, m), 7.29-7.43 (2H, m), 9.61 (1H, s)

Methyl 4-amino-2-(3-chlorophenyl)-5-cyano-pyrazole-3-carboxylate

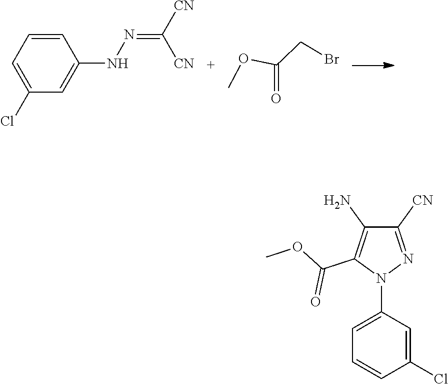

Methyl 2-bromoacetate (2.0 mL, 21.3 mmol) was added to a stirring mixture of 2-[(3-chlorophenyl) hydrazono]propanedinitrile (870 mg, 4.25 mmol) and K₂C₀3, 325 mesh (1590 mg, 11.5 mmol) in 1,4-dioxane (20 mL) at rt. The mixture was heated to 100° C. and stirred for 20 h. The solvent was removed, the residue treated with water (50 mL) and extracted with EtOAc. The organic fractions were combined, washed with saturated brine (50 mL), dried (Na₂SO₄), filtered and concentrated to afford the product (1525 mg). A portion of the product (195 mg) was purified by flash chromatography (silica, 5-100% EtOAc in iso-hexane).

¹H NMR (DMSO-d6, 400 MHZ) δ 3.71 (3H, s), 6.12 (2H, s), 7.44-7.61 (3H, m), 7.63-7.72 (1H, m) 4-Amino-2-(3-chlorophenyl)-5-cyano-pyrazole-3-carboxylic acid

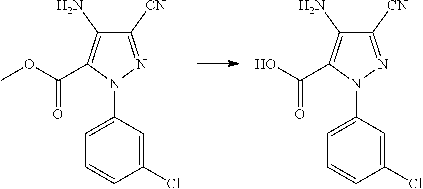 [SMILES: COC(=O)c1c(N)c(C#N)nn1-c1cccc(Cl)c1 ; N#Cc1nn(-c2cccc(Cl)c2)c(C(=O)O)c1N]

LiOH·H₂O (337 mg, 8.02 mmol) was added to a stirring solution of methyl 4-amino-2-(3-chlorophenyl)-5-cyano-pyrazole-3-carboxylate (740 mg, 2.67 mmol) in THF (2 mL) at 0° C. The mixture was stirred at rt for 3 h. The mixture was quenched carefully with 1M HCl (aq)(8 mL) until pH 3 was reached. The mixture was concentrated and azeotroped with toluene (10 mL) four times to afford the product (1060 mg).

[M−H]⁻=261.0/263.0

¹H NMR (DMSO-d6, 400 MHZ) δ 7.40-7.54 (3H, m), 7.58 (1H, t, J=2.0 Hz) tert-Butyl 7-[[4-amino-2-(3-chlorophenyl)-5-cyano-pyrazole-3-carbonyl]amino]-3,4-dihydro-1H-isoquinoline-2-carboxylate

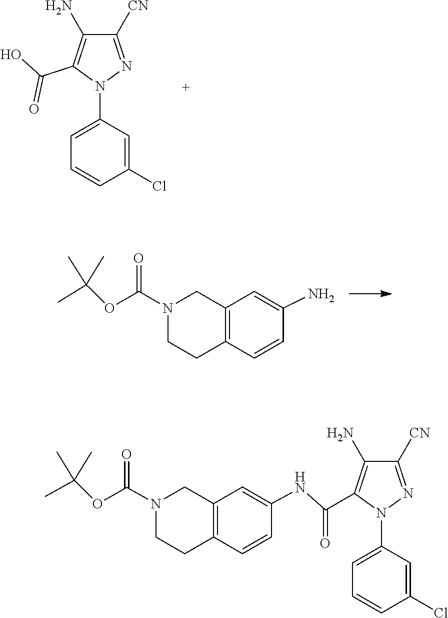 [SMILES: N#Cc1nn(-c2cccc(Cl)c2)c(C(=O)O)c1N ; CC(C)(C)OC(=O)N1CCc2ccc(N)cc2C1 ; CC(C)(C)OC(=O)N1CCc2ccc(NC(=O)c3c(N)c(C#N)nn3-c3cccc(Cl)c3)cc2C1]

HATU (1950 mg, 5.14 mmol) was added to a stirring mixture of 4-amino-2-(3-chlorophenyl)-5-cyano-pyrazole-3-carboxylic acid (750 mg, 2.86 mmol), tert-butyl 7-amino-3,4-dihydroisoquinoline-2 (1H)-carboxylate (1060 mg, 4.28 mmol) and DIPEA (2.5 mL) in DMF (15 mL) at rt. The mixture was stirred for 3 h. Brine (75 mL) was added and the mixture extracted with EtOAc (2×50 mL). The organic fractions were combined, washed with sat. NaHCO₃(aq) and saturated brine solution, dried (Na₂SO₄), filtered and concentrated. Flash chromatography (silica, 0-100% EtOAc in iso-hexane) afforded the product (615 mg, 44% yield).

¹H NMR (CDCl₃, 400 MHZ) δ 1.42 (9H, s), 2.72 (2H, t, J=5.9 Hz), 3.52 (2H, t, J=5.9 Hz), 4.43 (2H, s), 5.54 (2H, s), 7.10 (1H, d, J=8.3 Hz), 7.23 (1H, s), 7.35 (1H, s), 7.36-7.46 (1H, m), 7.46-7.55 (2H, m), 7.57 (1H, s), 10.17 (1H, s)

[M−H]⁻=491.3/493.3

### tert-Butyl 7-[1-(3-chlorophenyl)-3-cyano-5-methyl-7-oxo-pyrazolo[4,3-d]pyrimidin-6-yl]-3,4-dihydro-1H-isoquinoline-2-carboxylate

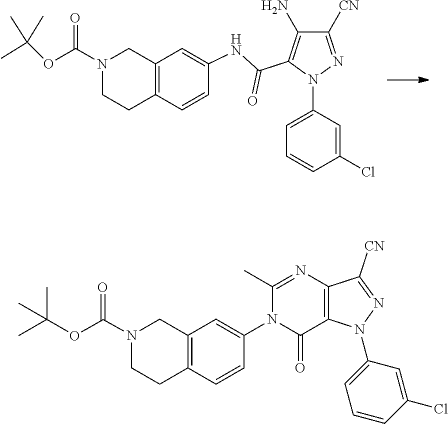 [SMILES: CC(C)(C)OC(=O)N1CCc2ccc(NC(=O)c3c(N)c(C#N)nn3-c3cccc(Cl)c3)cc2C1 ; Cc1nc2c(C#N)nn(-c3cccc(Cl)c3)c2c(=O)n1-c1ccc2c(c1)CN(C(=O)OC(C)(C)C)CC2]

Triethyl orthoacetate (0.19 mL, 1.01 mmol) was added to a stirring solution of tert-butyl 7-[[4-amino-2-(3-chlorophenyl)-5-cyano-pyrazole-3-carbonyl]amino]-3,4-dihydro-1H-isoquinoline-2-carboxylate (50 mg, 0.101 mmol) in AcOH (0.5 mL) at rt. The mixture was heated to 100° C., stirred for 2 h and allowed to cool. Sat. NaHCO₃(aq)(10 mL) was added and the mixture extracted with DCM (3×5 mL). The organic fractions were combined, washed with sat. brine solution, dried (Na₂SO₄), filtered and concentrated to afford the product (52 mg, 99% yield).

[M+H]⁺=517.3/519.3

¹H NMR (DMSO-d6, 400 MHZ) δ 1.43 (9H, s), 2.22 (3H, s), 2.86 (2H, t, J=5.9 Hz), 3.55-3.60 (2H, m), 4.41 -4.62 (2H, m), 7.17-7.26 (2H, m), 7.37 (1H, d, J=7.9 Hz), 7.54-7.62 (1H, m), 7.62 (1H, dt, J=8.0, 1.7 Hz), 7.72 (1H, dt, J=7.5, 1.8 Hz), 7.87 (1H, t, J=2.0 Hz)

### 1-(3-Chlorophenyl)-5-methyl-7-oxo-6-(1,2,3,4-tetrahydroisoquinolin-7-yl) pyrazolo[4,3-d]pyrimidine-3-carbonitrile hydrochloride

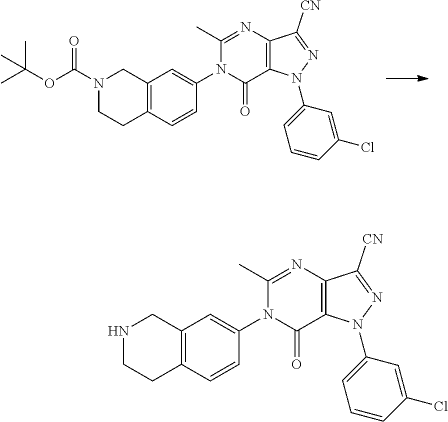 [SMILES: Cc1nc2c(C#N)nn(-c3cccc(Cl)c3)c2c(=O)n1-c1ccc2c(c1)CN(C(=O)OC(C)(C)C)CC2 ; Cc1nc2c(C#N)nn(-c3cccc(Cl)c3)c2c(=O)n1-c1ccc2c(c1)CNCC2]

HCl solution (4M in 1,4-dioxane, 0.50 mL) was added to a stirring solution of tert-butyl 7-[1-(3-chlorophenyl)-3-cyano-5-methyl-7-oxo-pyrazolo[4,3-d]pyrimidin-6-yl]-3,4-dihydro-1H-isoquinoline-2-carboxylate (52 mg, 0.10 mmol) in 1,4-dioxane (0.5 mL) at rt and stirred for 2 h. The reaction solvent was removed to afford the product (61 mg).

[M+H]⁺=417.3/419.3

### 1-(3-Chlorophenyl)-5-methyl-6-(2-methyl-1,2,3,4-tetrahydroisoquinolin-7-yl)-7-oxo-6,7-dihydro-1H-pyrazolo[4,3-d]pyrimidine-3-carbonitrile

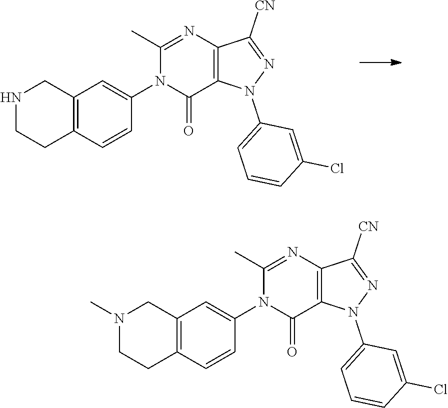 [SMILES: Cc1nc2c(C#N)nn(-c3cccc(Cl)c3)c2c(=O)n1-c1ccc2c(c1)CNCC2 ; Cc1nc2c(C#N)nn(-c3cccc(Cl)c3)c2c(=O)n1-c1ccc2c(c1)CN(C)CC2]

Formaldehyde, 37 wt. % in H₂O, (0.037 mL, 0.50 mmol) was added to a stirring solution of 1-(3-chlorophenyl)-5-methyl-7-oxo-6-(1,2,3,4-tetrahydroisoquinolin-7-yl) pyrazolo[4,3-d]pyrimidine-3-carbonitrile hydrochloride (61 mg, 0.13 mmol) and TEA (0.069 mL, 0.50 mmol) in a mixture of DCM (1 mL) and MeOH (0.2 mL) at rt and stirred for 15 min. STAB (53 mg, 0.25 mmol) was added and the mixture stirred at rt for 2 h. Sat. K₂CO₃ (aq)(5 mL) was added and the mixture was extracted with DCM (3×5 mL). The organic fractions were combined, dried (Na₂SO₄), filtered and concentrated. Flash chromatography (silica, 0-15% MeOH in DCM) and lyophilisation afforded the product (9.0 mg, 17% yield).

[M+H]⁺=431.1/433.1

¹H NMR (DMSO-d6, 400 MHZ) δ 2.21 (3H, s), 2.35 (3H, s), 2.55-2.72 (2H, m), 2.89 (2H, s), 3.34-3.61 (2H, m), 7.09 (1H, d, J=2.1 Hz), 7.15 (1H, dd, J=8.0, 2.2 Hz), 7.30 (1H, d, J=8.1 Hz), 7.54-7.66 (2H, m), 7.69-7.76 (1H, m), 7.87 (1H, t, J=1.9 Hz)

### Example Number 1217

### 1-(3-Chlorophenyl)-N-[(1,4-dimethylpyrazol-3-yl)methyl]-6-[2-(2-hydroxyethyl)-1-oxo-3,4-dihydroisoquinolin-7-yl]-7-oxo-4,5-dihydropyrazolo[3,4-c]pyridine-3-carboxamide

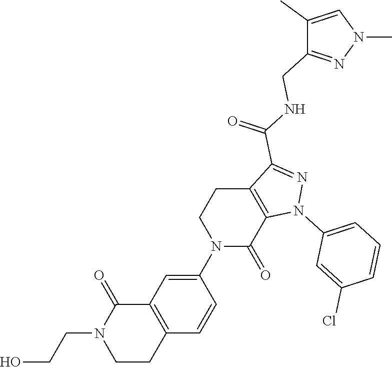 [SMILES: Cc1cn(C)nc1CNC(=O)c1nn(-c2cccc(Cl)c2)c2c1CCN(c1ccc3c(c1)C(=O)N(CCO)CC3)C2=O]

### 7-Bromo-2-[2-[tert-butyl(dimethyl) silyl]oxyethyl]-3,4-dihydroisoquinolin-1-one

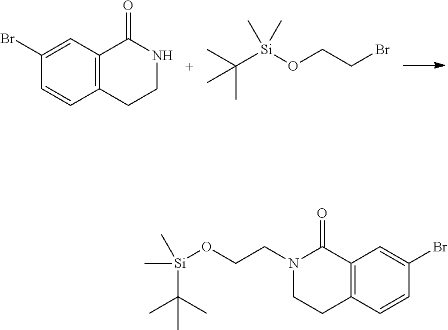 [SMILES: O=C1NCCc2ccc(Br)cc21 ; CC(C)(C)[Si](C)(C)OCCBr ; CC(C)(C)[Si](C)(C)OCCN1CCc2ccc(Br)cc2C1=O]

To a solution of 7-bromo-3,4-dihydro-2h-isoquinolin-1-one (1000 mg, 4.42 mmol) in DMF (40 mL) was added NaH (60% in mineral oil, 230 mg, 5.75 mmol) and the mixture was stirred at rt for 1 h. 2-Bromoethoxy-t-butyldimethylsilane (1.2 mL, 5.75 mmol) was added dropwise and the reaction stirred for 20 h. The solution was then quenched with sat. NH₄Cl (aq)(25 mL) and diluted with EtOAc (150 mL). The EtOAc layer was washed sat NH₄Cl (aq)(25 mL) and brine (3×50 mL), dried (Na₂SO₄), filtered and concentrated. Flash column chromatography (silica, 5-30% EtOAc in iso-hexane) afforded the product (1280 mg, 75% yield).

[M+H]⁺=384.1/386.1

¹H NMR (DMSO-d6, 400 MHZ) δ 0.02 (6H, s), 0.84 (9H, s), 2.92 (2H, t, J=6.6 Hz), 3.56 (2H, t, J=5.7 Hz), 3.62 (2H, t, J=6.3 Hz), 3.76 (2H, t, J=5.7 Hz), 7.28 (1H, dd, J=8.1, 0.5 Hz), 7.66 (1H, dd, J=8.1, 2.3 Hz), 7.92 (1H, d, J=2.1 Hz).

### Ethyl 6-[2-[2-[tert-butyl(dimethyl) silyl]oxyethyl]-1-oxo-3,4-dihydroisoquinolin-7-yl]-1-(3-chlorophenyl)-7-oxo-4,5-dihydropyrazolo[3,4-c]pyridine-3-carboxylate

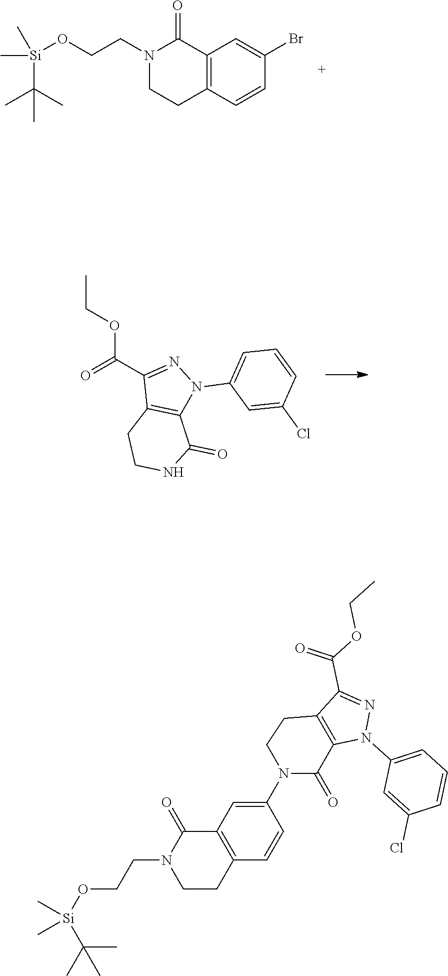 [SMILES: CC(C)(C)[Si](C)(C)OCCN1CCc2ccc(Br)cc2C1=O ; CCOC(=O)c1nn(-c2cccc(Cl)c2)c2c1CCNC2=O ; CCOC(=O)c1nn(-c2cccc(Cl)c2)c2c1CCN(c1ccc3c(c1)C(=O)N(CCO[Si](C)(C)C(C)(C)C)CC3)C2=O]

A solution of ethyl 1-(3-chlorophenyl)-7-oxo-5,6-dihydro-4H-pyrazolo[3,4-c]pyridine-3-carboxylate (1273 mg, 3.98 mmol) and 7-bromo-2-[2-[tert-butyl(dimethyl) silyl]oxyethyl]-3,4-dihydroisoquinolin-1-one (1275 mg, 3.32 mmol) in dry 1,4-dioxane (35 mL) was bubbled with N₂ (g) for 10 min. Tripotassium phosphate (2112 mg, 9.95 mmol), (1S,2S)—N,N′-dimethyl-1,2-cyclohexanediamine (377 mg, 2.65 mmol) and Cu(I)I (253 mg, 1.33 mmol) were added sequentially. The system was closed and stirred at 110° C. for 20 h. The solution was filtered through Celite®, rinsing with EtOAc (120 mL), and the filtrate was washed with a sat. NaHCO₃(aq)(2×25 mL) and brine (25 mL), dried (Na₂SO₄), filtered and concentrated. Flash chromatography (silica, 1-50% EtOAc in iso-hexane) afforded the product (690 mg, 33% yield).

[M+H]⁺=623.3/625.3

¹H NMR (DMSO-d6, 400 MHZ) δ 0.03 (6H, s), 0.85 (9H, s), 1.33 (3H, t, J=7.1 Hz), 2.95 (2H, t, J=6.5 Hz), 3.21 (2H, t, J=6.6 Hz), 3.57 (2H, t, J=5.8 Hz), 3.62 (2H, t, J=6.6 Hz), 3.76 (2H, t, J=5.8 Hz), 4.09 (2H, t, J=6.6 Hz), 4.35 (2H, q, J=7.1 Hz), 7.32 (1H, d, J=8.1 Hz), 7.45 (1H, dd, J=8.1, 2.3 Hz), 7.49-7.58 (2H, m), 7.60 (1H, dt, J=7.3, 1.9 Hz), 7.76 (1H, td, J=2.0, 0.6 Hz), 7.83 (1H, d, J=2.4 Hz).

### 1-(3-Chlorophenyl)-6-[2-(2-hydroxyethyl)-1-oxo-3,4-dihydroisoquinolin-7-yl]-7-oxo-4,5-dihydropyrazolo[3,4-c]pyridine-3-carboxylic

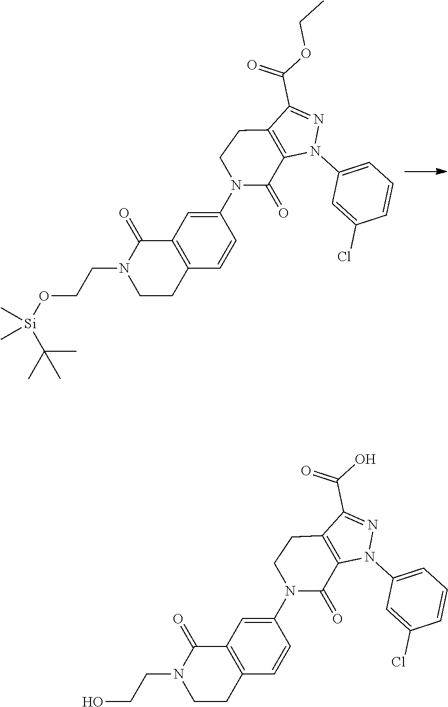 [SMILES: CCOC(=O)c1nn(-c2cccc(Cl)c2)c2c1CCN(c1ccc3c(c1)C(=O)N(CCO[Si](C)(C)C(C)(C)C)CC3)C2=O ; O=C(O)c1nn(-c2cccc(Cl)c2)c2c1CCN(c1ccc3c(c1)C(=O)N(CCO)CC3)C2=O]

To a solution of ethyl 6-[2-[2-[tert-butyl(dimethyl) silyl]oxyethyl]-1-oxo-3,4-dihydroisoquinolin-7-yl]-1-(3-chlorophenyl)-7-oxo-4,5-dihydropyrazolo[3,4-c]pyridine-3-carboxylate (685 mg, 1.10 mmol), in a mixture of THF (8 mL) and water (4 mL), was added LiOH·H₂O (115 mg, 2.75 mmol), and the mixture was stirred at rt for 20 h. The mixture was acidified with 4M HCl solution in dioxane (2 mL), stirred for 1 h and evaporated to provide the product (811 mg, 100% yield).

[M+H]⁺=481.2/483.

### 1-(3-Chlorophenyl)-N-[(1,4-dimethylpyrazol-3-yl)methyl]-6-[2-(2-hydroxyethyl)-1-oxo-3,4-dihydroisoquinolin-7-yl]-7-oxo-4,5-dihydropyrazolo[3,4-c]pyridine-3-carboxamide

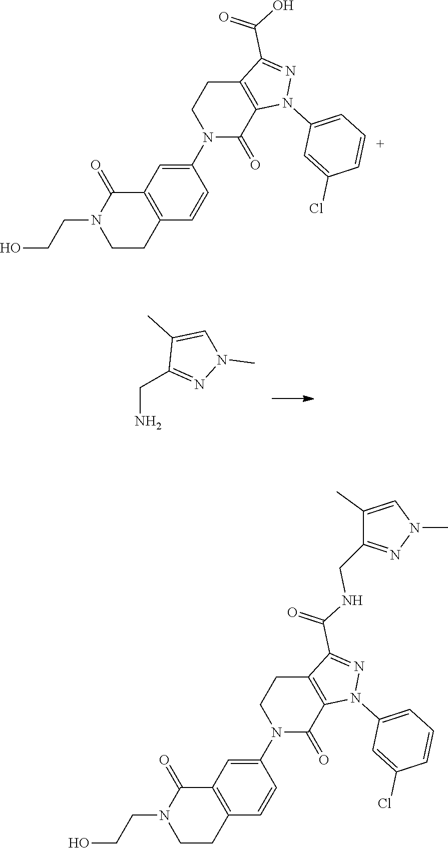

To a solution of 1-(3-chlorophenyl)-6-[2-(2-hydroxyethyl)-1-oxo-3,4-dihydroisoquinolin-7-yl]-7-oxo-4,5-dihydropyrazolo[3,4-c]pyridine-3-carboxylic acid (60 mg, 0.125 mmol) in DMF (2 mL) were added 1-(1,4-dimethyl-1H-pyrazol-3-yl) methanamine dihydrochloride (42 mg, 0.212 mmol), DIPEA (0.13 mL, 0.749 mmol) and HATU (71 mg, 0.187 mmol) and the mixture was stirred at rt for 18 h. The solution was diluted with EtOAc (30 mL) and washed with sat. NaHCO₃(aq)(15 mL), brine (15 mL), dried (Na₂SO₄), filtered and concentrated. The residue was purified by flash chromatography (silica, 0-10% MeOH in DCM) and loaded onto an SCX in DCM and washed with DCM (10 mL). The product was eluted with a solution of 3M NH₃ in MeOH and lyophilised to yield the product (42 mg, 55% yield).

[M+H]⁺=588.2/590.2

¹H NMR (400 MHZ, DMSO-d6) δ 1.98 (3H, s), 2.88-3.02 (2H, m), 3.17-3.26 (2H, m), 3.51-3.64 (6H, m), 3.72 (3H, m), 4.00-4.15 (2H, m), 4.30-4.43 (2H, m), 4.73-4.81 (1H, m), 7.30-7.34 (1H, m), 7.38 (1H, s), 7.41-7.56 (3H, m), 7.61-7.69 (1H, m), 7.78-7.85 (2H, m), 8.49-8.58 (1H, m)

### Example Number 1195

### 1-(3-Chlorophenyl)-N-(cyclopropylmethyl)-6-[3-(dimethylcarbamoyl)phenyl]-7-oxo-4,5-dihydropyrazolo[3,4-c]pyridine-3-carboxamide

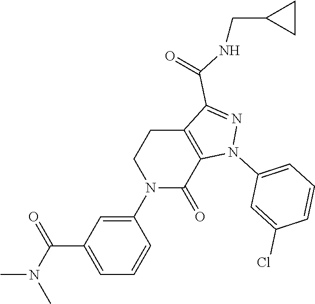 [SMILES: CN(C)C(=O)c1cccc(N2CCc3c(C(=O)NCC4CC4)nn(-c4cccc(Cl)c4)c3C2=O)c1]

### Ethyl 1-(3-chlorophenyl)-6-[3-(dimethylcarbamoyl)phenyl]-7-oxo-4,5-dihydropyrazolo[3,4-c]pyridine-3-carboxylate

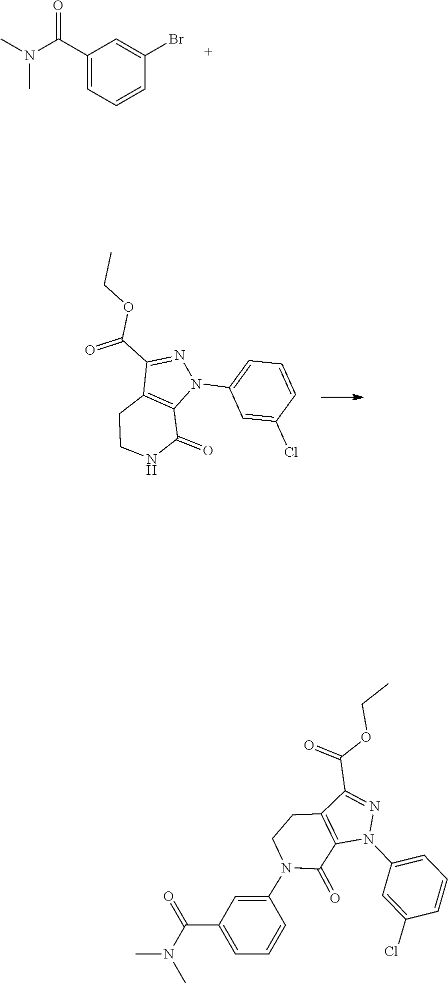 [SMILES: CN(C)C(=O)c1cccc(Br)c1 ; CCOC(=O)c1nn(-c2cccc(Cl)c2)c2c1CCNC2=O ; CCOC(=O)c1nn(-c2cccc(Cl)c2)c2c1CCN(c1cccc(C(=O)N(C)C)c1)C2=O]

A pressure vial was charged with Cu(I)I (48 mg, 0.25 mmol), tripotassium phosphate (269 mg, 1.25 mmol), ethyl 1-(3-chlorophenyl)-4,5,6,7-tetrahydro-7-oxo-1H-pyrazolo[3,4-c]pyridine-3-carboxylate (200 mg, 0.63 mmol) and 3-bromo-N,N-dimethylbenzamide (157 mg, 0.69 mmol). The flask was sealed, evacuated and backfilled with N₂ (g). N,N′ Dimethylethylenediamine (35 μL, 0.25 mmol) and toluene (5 mL) were added. The mixture was purged with N₂ (g) for 10 min and stirred at 100° C. for 22 h. The mixture was filtered through Celite®, washing with EtOAc (100 mL), DCM (100 mL) and MeOH (10 mL). The filtrate was concentrated and the residue was purified via flash chromatography (silica, 0-100% EtOAc in Pet. Ether) to afford the product (100 mg, 34% yield).

[M+H]⁺=467.1

¹H NMR (DMSO-d6, 400 MHZ) δ 1.33 (3H, t, J=7.1 Hz), 2.92 (3H, s), 2.97 (3H, s), 3.22 (2H, t, J=6.6 Hz), 4.12 (2H, t, J=6.6 Hz), 4.36 (2H, q, J=7.1 Hz), 7.25-7.30 (1H, m), 7.40-7.49 (3H, m), 7.50-7.55 (1H, m), 7.56 (1H, dt, J=8.0, 1.7 Hz), 7.61 (1H, dt, J=7.4, 1.8 Hz), 7.76-7.79 (1H, m)

### 1-(3-Chlorophenyl)-N-(cyclopropylmethyl)-6-[3-(dimethylcarbamoyl)phenyl]-7-oxo-4,5-dihydropyrazolo[3,4-c]pyridine-3-carboxamide

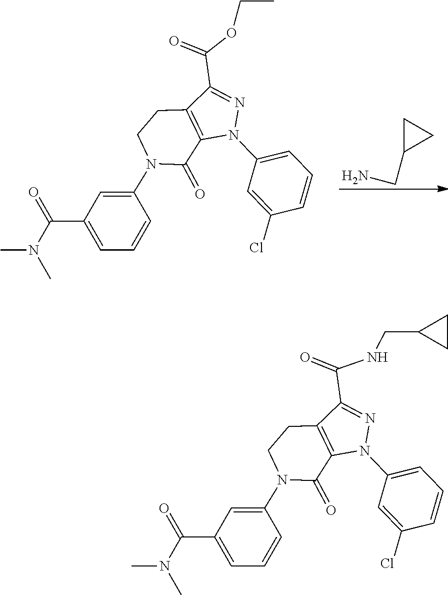 [SMILES: CCOC(=O)c1nn(-c2cccc(Cl)c2)c2c1CCN(c1cccc(C(=O)N(C)C)c1)C2=O ; NCC1CC1 ; CN(C)C(=O)c1cccc(N2CCc3c(C(=O)NCC4CC4)nn(-c4cccc(Cl)c4)c3C2=O)c1]

Trimethylaluminium, 2M in toluene, (80 μL, 0.16 mmol) was added to a solution of cyclopropylmethylamine (14 μL, 0.16 mmol) in 1,4-dioxane (1 mL). The mixture was stirred at rt for 30 min and ethyl 1-(3-chlorophenyl)-6-[3-(dimethylcarbamoyl)phenyl]-7-oxo-4,5-dihydropyrazolo[3,4-c]pyridine-3-carboxylate (50 mg, 0.11 mmol) in 1,4-dioxane (1 mL) was added slowly dropwise (exothermic). After stirring at rt for 5 min the reaction temperature increased to 90° C. After stirring for 22 h the reaction was partitioned between sat. NaHCO₃(aq)(30 mL) and DCM (30 mL). The organic layer was separated and the aqueous layer extracted with further DCM (20 mL). The combined DCM extracts were dried (MgSO₄), filtered and concentrated. Flash chromatography (silica, 0-100% of EtOAc in Pet. Ether) afforded the product (18 mg, 34% yield).

[M+H]⁺=492.1

¹H NMR (DMSO-d6, 400 MHZ) δ 0.19-0.26 (2H, m), 0.34-0.48 (2H, m), 0.98-1.10 (1H, m), 2.92 (3H, s), 2.97 (3H, s), 3.12 (2H, t, J=6.5 Hz), 3.22 (2H, t, J=6.6 Hz), 4.09 (2H, t, J=6.6 Hz), 7.26-7.31 (1H, m), 7.40 -7.50 (3H, m), 7.50-7.56 (2H, m), 7.64 (1H, dt, J=6.9, 2.1 Hz), 7.79-7.82 (1H, m), 8.56 (1H, t, J=6.0 Hz)

### Example Number 3009

### N-[[1-(3-Chlorophenyl)-6-[2-(2-hydroxyethyl)-3,4-dihydro-1H-isoquinolin-7-yl]-5-methyl-7-oxopyrazolo[4,3-d]pyrimidin-3-yl]methyl]-1,4-dimethylpyrazole-3-carboxamide

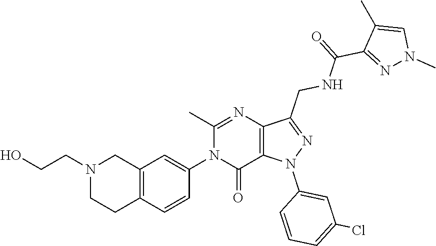 [SMILES: Cc1cn(C)nc1C(=O)NCc1nn(-c2cccc(Cl)c2)c2c(=O)n(-c3ccc4c(c3)CN(CCO)CC4)c(C)nc12]

### 6-[2-[2-[tert-butyl(dimethyl) silyl]oxyethyl]-3,4-dihydro-1H-isoquinolin-7-yl]-1-(3-chlorophenyl)-5-methyl-7-oxo-pyrazolo[4,3-d]pyrimidine-3-carbonitrile

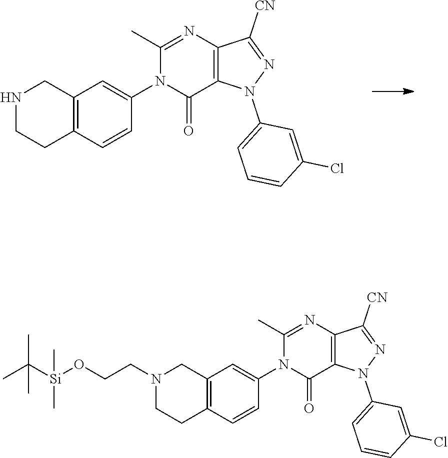 [SMILES: Cc1nc2c(C#N)nn(-c3cccc(Cl)c3)c2c(=O)n1-c1ccc2c(c1)CNCC2 ; Cc1nc2c(C#N)nn(-c3cccc(Cl)c3)c2c(=O)n1-c1ccc2c(c1)CN(CCO[Si](C)(C)C(C)(C)C)CC2]

2-Bromoethoxy-t-butyldimethylsilane (0.99 mL, 4.63 mmol) was added to a solution of 1-(3-chlorophenyl)-5-methyl-7-oxo-6-(1,2,3,4-tetrahydroisoquinolin-7-yl) pyrazolo[4,3-d]pyrimidine-3-carbonitrile dihydrochloride (1.13 g, 2.32 mmol) and TEA (1.6 mL, 11.6 mmol) in DMF (12 mL). After stirring at rt for 3 days, sat. K₂CO₃ (aq)(10 mL) was added and the mixture extracted with EtOAc (2×100 mL). The combined organic extracts were washed with brine (3×150 mL), dried (Na₂SO₄), filtered and evaporated. Flash chromatography (silica, 0-100% EtOAc in iso-hexane) afforded the product (726 mg, 51% yield).

[M+H]⁺=575.2/577.2

¹H NMR (CDCl₃, 400 MHZ) δ 0.07 (6H, s), 0.90 (9H, s), 2.30 (3H, s), 2.70-2.74 (4H, m), 2.99-3.04 (2H, m), 3.65-3.87 (4H, m), 6.86 (1H, s), 6.97 (1H, d, J=8.1 Hz), 7.30 (1H, d, J=8.1 Hz), 7.35-7.46 (2H, m), 7.61-7.70 (1H, m), 7.76-7.81 (1H, m)

### tert-Butyl N-[[6-[2-[2-[tert-butyl(dimethyl) silyl]oxyethyl]-3,4-dihydro-1H-isoquinolin-7-yl]-1-(3-chlorophenyl)-5-methyl-7-oxo-pyrazolo[4,3-d]pyrimidin-3-yl]methyl]carbamate

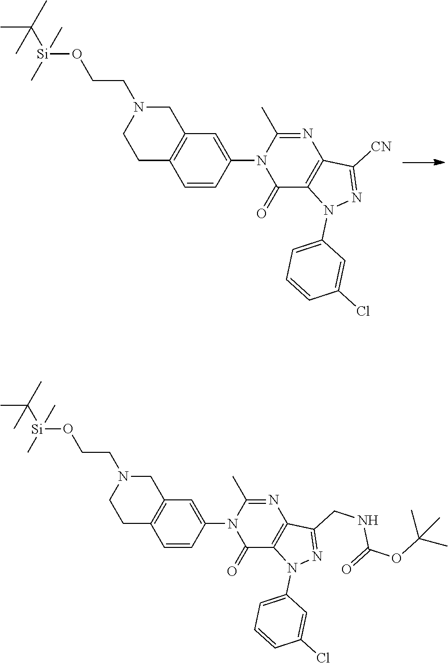 [SMILES: Cc1nc2c(C#N)nn(-c3cccc(Cl)c3)c2c(=O)n1-c1ccc2c(c1)CN(CCO[Si](C)(C)C(C)(C)C)CC2 ; Cc1nc2c(CNC(=O)OC(C)(C)C)nn(-c3cccc(Cl)c3)c2c(=O)n1-c1ccc2c(c1)CN(CCO[Si](C)(C)C(C)(C)C)CC2]

NaBH₄ (334 mg, 8.84 mmol) was added over a period of 25 min to a solution of 6-[2-[2-[tert-butyl (dimethyl) silyl]oxyethyl]-3,4-dihydro-1H-isoquinolin-7-yl]-1-(3-chlorophenyl)-5-methyl-7-oxo-pyrazolo[4,3-d]pyrimidine-3-carbonitrile (726 mg, 1.26 mmol), Boc₂O (413 mg, 1.89 mmol) and Ni (II) Cl₂·6H₂O (30 mg, 0.126 mmol) in MeOH (6 mL) at while cooling in an ice/water bath. The mixture was stirred while continuing to cool for 4 h. THF (6 mL) was added, followed by the addition of NaBH₄ (334 mg, 8.84 mmol) over a period of 25 min. The mixture was stirred while cooling in an ice/water bath for 2 h. The reaction was quenched with water (10 mL), the organic solvent was evaporated, and the residue was extracted with EtOAc (2×100 mL). The combined organic extracts were washed with brine (3×150 mL), dried (Na₂SO₄), filtered and evaporated. Flash chromatography (silica, 0-100% EtOAc in iso-hexane) afforded the product (299 mg, 31% yield).

[M+H]⁺=679.6/681.5

¹H NMR (CDCl₃, 400 MHZ) δ 0.07 (6H, s), 0.86-0.95 (9H, m), 1.49 (9H, s), 2.24 (3H, s), 2.70-2.74 (2H, m), 2.86-3.04 (4H, m), 3.65-3.87 (4H, m), 4.73 (2H, d, J=5.6 Hz), 5.48 (1H, s), 6.86 (1H, s), 6.98 (1H, s), 7.24 -7.37 (3H, m), 7.64-7.71 (1H, m), 7.79 (1H, t, J=2.0 Hz).

### 3-(Aminomethyl)-1-(3-chlorophenyl)-6-[2-(2-hydroxyethyl)-3,4-dihydro-1H-isoquinolin-7-yl]-5-methyl-pyrazolo[4,3-d]pyrimidin-7-one dihydrochloride

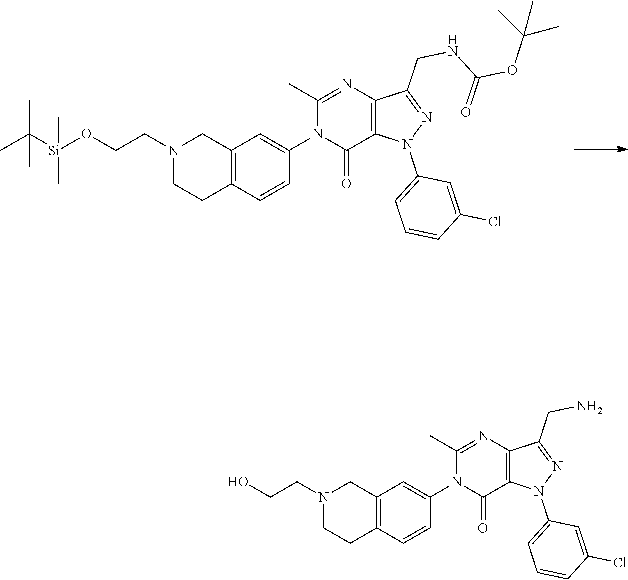 [SMILES: Cc1nc2c(CNC(=O)OC(C)(C)C)nn(-c3cccc(Cl)c3)c2c(=O)n1-c1ccc2c(c1)CN(CCO[Si](C)(C)C(C)(C)C)CC2 ; Cc1nc2c(CN)nn(-c3cccc(Cl)c3)c2c(=O)n1-c1ccc2c(c1)CN(CCO)CC2]

4M HCl in 1,4-dioxane (2.0 mL, 8.00 mmol) was added to a solution of tert-butyl N-[[6-[2-[2-[tert-butyl (dimethyl) silyl]oxyethyl]-3,4-dihydro-1H-isoquinolin-7-yl]-1-(3-chlorophenyl)-5-methyl-7-oxo-pyrazolo[4,3-d]pyrimidin-3-yl]methyl]carbamate (299 mg, 0.39 mmol) in 1,4-dioxane (2 mL). After 3 h at rt the mixture was evaporated to afford the product (360 mg).

[M+H]⁺=465.3/467.3

¹H NMR (DMSO-d6, 400 MHZ) δ 2.19 (3H, d, J=2.8 Hz), 3.14-3.19 (2H, m), 3.27-3.36 (2H, m), 3.86 (2H, t, J=5.1 Hz), 4.38-4.48 (4H, m), 4.61 (2H, t, J=15.3 Hz), 7.28-7.38 (2H, m), 7.43-7.61 (3H, m), 7.70-7.78 (1H, m), 7.86 (1H, d, J=2.0 Hz), 8.61-8.65 (3H, m), 10.55-10.59 (1H, m)

### N-[[1-(3-Chlorophenyl)-6-[2-(2-hydroxyethyl)-3,4-dihydro-1H-isoquinolin-7-yl]-5-methyl-7-oxopyrazolo[4,3-d]pyrimidin-3-yl]methyl]-1,4-dimethylpyrazole-3-carboxamide

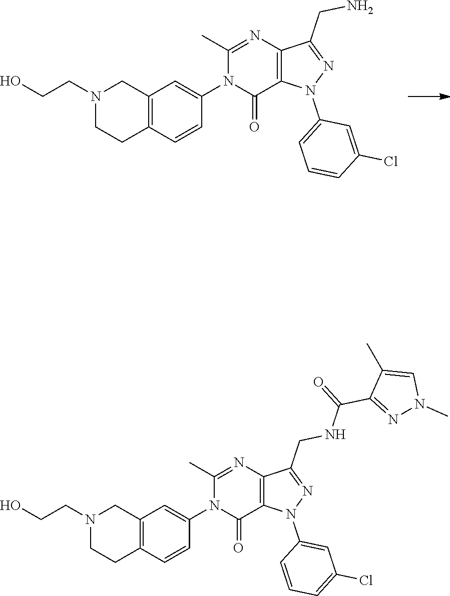 [SMILES: Cc1nc2c(CN)nn(-c3cccc(Cl)c3)c2c(=O)n1-c1ccc2c(c1)CN(CCO)CC2 ; Cc1cn(C)nc1C(=O)NCc1nn(-c2cccc(Cl)c2)c2c(=O)n(-c3ccc4c(c3)CN(CCO)CC4)c(C)nc12]

HATU (71 mg, 0.19 mmol) was added to a solution of 3-(aminomethyl)-1-(3-chlorophenyl)-6-[2-(2-hydroxyethyl)-3,4-dihydro-1H-isoquinolin-7-yl]-5-methyl-pyrazolo[4,3-d]pyrimidin-7-one dihydrochloride (90 mg, 0.12 mmol), 1,4-dimethyl-1H-pyrazole-3-carboxylic acid (17 mg, 0.12 mmol) and DIPEA (0.13 mL, 0.75 mmol) in DMF (1 mL). After 3 h at rt, sat. K₂CO₃ (aq)(10 mL) was added and the mixture extracted with EtOAc (2×50 mL). The combined organic extracts were washed with brine (3×20 mL), dried (Na₂SO₄), filtered and evaporated. Flash chromatography (silica, 0-15% MeOH in DCM) and further purification by preparative HPLC (AccQPrep, 30-60% MeCN containing 0.1% NH₃, in 0.01 M aqueous ammonium bicarbonate) afforded the product (16 mg, 22% yield).

[M+H]⁺=587.2/589.2

¹H NMR (DMSO-d6, 400 MHZ) δ 2.17 (3H, s), 2.18-2.21 (3H, m), 2.57 (2H, t, J=6.2 Hz), 2.65-2.84 (2H, m), 2.84-2.90 (2H, m), 3.54-3.72 (4H, m), 3.82 (3H, s), 4.50 (1H, t, J=5.4 Hz), 4.76 (2H, d, J=5.8 Hz), 7.07 -7.12 (1H, m), 7.12-7.18 (1H, m), 7.28 (1H, d, J=8.1 Hz), 7.42-7.54 (2H, m), 7.54-7.59 (1H, m), 7.62-7.69 (1H, m), 7.74-7.80 (1H, m), 8.31 (1H, t, J=5.7 Hz)

### Example Number 3007

### N-[[1-(3-Chlorophenyl)-6-[2-(2-hydroxyethyl)-3,4-dihydro-1H-isoquinolin-7-yl]-5-methyl-7-oxopyrazolo[4,3-d]pyrimidin-3-yl]methyl]cyclopropanesulfonamide

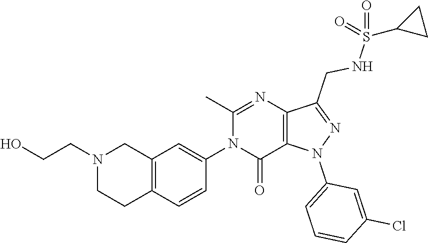 [SMILES: Cc1nc2c(CNS(=O)(=O)C3CC3)nn(-c3cccc(Cl)c3)c2c(=O)n1-c1ccc2c(c1)CN(CCO)CC2]

### N-[[1-(3-Chlorophenyl)-6-[2-(2-hydroxyethyl)-3,4-dihydro-1H-isoquinolin-7-yl]-5-methyl-7-oxopyrazolo[4,3-d]pyrimidin-3-yl]methyl]cyclopropanesulfonamide

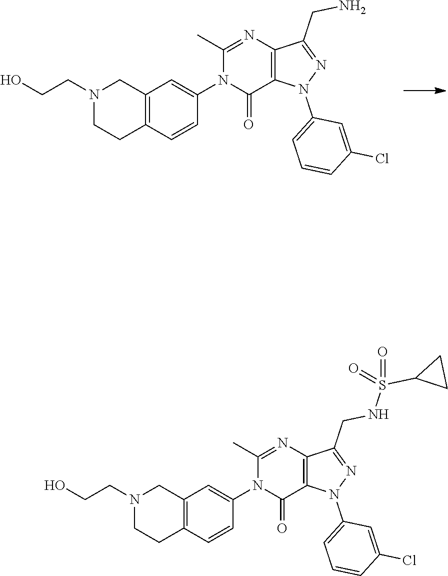 [SMILES: Cc1nc2c(CN)nn(-c3cccc(Cl)c3)c2c(=O)n1-c1ccc2c(c1)CN(CCO)CC2 ; Cc1nc2c(CNS(=O)(=O)C3CC3)nn(-c3cccc(Cl)c3)c2c(=O)n1-c1ccc2c(c1)CN(CCO)CC2]

Cyclopropanesulfonyl chloride (0.013 mL, 0.12 mmol) was added to a solution of 3-(aminomethyl)-1-(3-chlorophenyl)-6-[2-(2-hydroxyethyl)-3,4-dihydro-1H-isoquinolin-7-yl]-5-methyl-pyrazolo[4,3-d] pyrimidin-7-one dihydrochloride (90 mg, 0.12 mmol) and TEA (0.069 mL, 0.50 mmol) in DCM (1.5 mL). After 3 h at rt, sat. K₂CO₃ (aq)(10 mL) was added and the mixture extracted with EtOAc (2×50 mL). The combined organic extracts were washed with brine (3×20 mL), dried (Na₂SO₄), filtered and evaporated. Purification by flash chromatography (silica, 0-15% MeOH in DCM) followed by SCX-2 ion-exchange, loading with MeOH (10 mL) and eluting with 2M NH₃ in MeOH (10 mL), afforded the product (16 mg, 23% yield).

[M+H]⁺=569.3/571.3

¹H NMR (DMSO-d6, 400 MHZ) δ 0.85-0.99 (4H, m), 2.17 (3H, s), 2.54-2.83 (5H, m), 2.86-2.90 (2H, m), 3.54-3.73 (4H, m), 4.49-4.55 (3H, m), 7.08-7.12 (1H, m), 7.12-7.19 (1H, m), 7.28 (1H, d, J=8.1 Hz), 7.46 -7.57 (2H, m), 7.62-7.76 (2H, m), 7.79-7.85 (1H, m)

### Example Number 3005

### 1-(3-Chlorophenyl)-N-[(1,4-dimethylpyrazol-3-yl)methyl]-6-[2-(2-hydroxyethyl)-3,4-dihydro-1H-isoquinolin-7-yl]-5-methyl-7-oxo-pyrazolo[4,3-d]pyrimidine-3-carboxamide

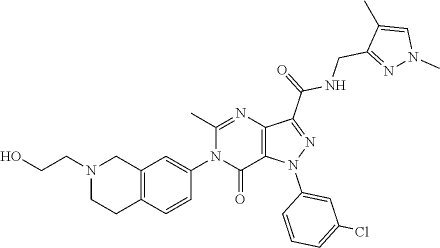 [SMILES: Cc1cn(C)nc1CNC(=O)c1nn(-c2cccc(Cl)c2)c2c(=O)n(-c3ccc4c(c3)CN(CCO)CC4)c(C)nc12]

### Ethyl 2-[(3-chlorophenyl) hydrazono]-2-cyano-acetate

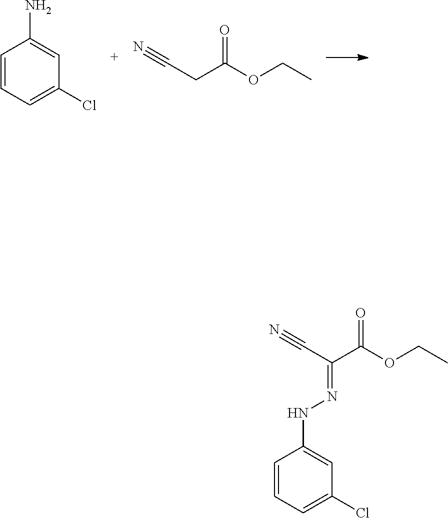

HCl (aq)(11.5M, 15 mL, 172 mmol) was added to a suspension of 3-chloroaniline (1.7 mL, 15.7 mmol) in water (40 mL) while cooling in an ice/water bath. 1M NaNO₂ (aq)(16 mL, 16 mmol) was added and the mixture stirred while continuing to cool for 90 min. The reaction mixture was then slowly added to a stirring solution of ethyl cyanoacetate (2.5 mL, 23.5 mmol) and sodium acetate trihydrate (64.00 g, 470 mmol) in water (50 mL) at 0° C. over a period of 30 minutes. The resulting mixture was then stirred at 0° C. fora further 60 min. The mixture was filtered and the precipitate washed with water (5×10 ml). The precipitate was taken up in EtOAc (100 mL), washed with brine (100 mL), dried (Na₂SO₄) and evaporated to afford the product (3.92 g, 99% yield).

[M+H]⁺=250.1

¹H NMR (CDCl₃, 400 MHz) δ 1.41 (3H, t, J=7.1 Hz), 4.41 (2H, q, J=7.1 Hz), 7.11-7.22 (2H, m), 7.27-7.39 (1H, m), 7.393-7.45 (1H, m), 9.28 (1H, s)

### O-5-tert-Butyl O-3-ethyl 4-amino-1-(3-chlorophenyl) pyrazole-3,5-dicarboxylate

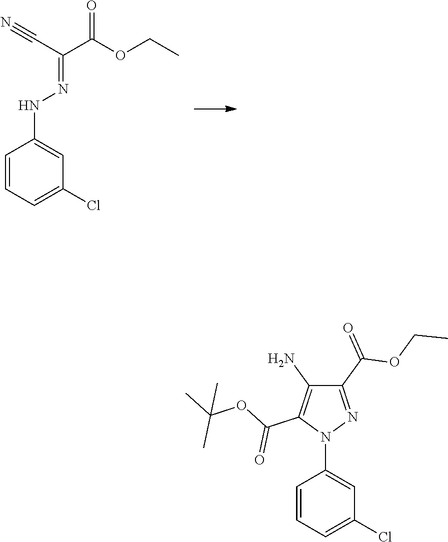 [SMILES: CCOC(=O)/C(C#N)=N/Nc1cccc(Cl)c1 ; CCOC(=O)c1nn(-c2cccc(Cl)c2)c(C(=O)OC(C)(C)C)c1N]

tert-Butyl bromoacetate (5.7 mL, 39 mmol) was added to a stirring suspension of ethyl 2-[(3-chlorophenyl) hydrazono]-2-cyano-acetate (3.92 g, 15.6 mmol) and K₂CO₃ (325 mesh, 5.80 g, 42.0 mmol) in 1,4-dioxane (80 mL). The mixture was heated to 100° C. for 20 h. The solvent was evaporated, water (250 mL) added and extracted into EtOAc (3×100 mL). The combined organic extracts were washed with brine (250 mL), dried (Na₂SO₄) and evaporated to afford the product (6.0 g, 94% yield).

[M+H]⁺=366.1/368.1

¹H NMR (CDCl₃, 400 MHZ) δ 1.35 (9H, s), 1.41 (3H, t, J=7.1 Hz), 4.44 (2H, q, J=7.1 Hz), 5.46 (2H, s), 7.26-7.43 (4H, m)

### 4-Amino-2-(3-chlorophenyl)-5-ethoxycarbonyl-pyrazole-3-carboxylic acid hydrochloride

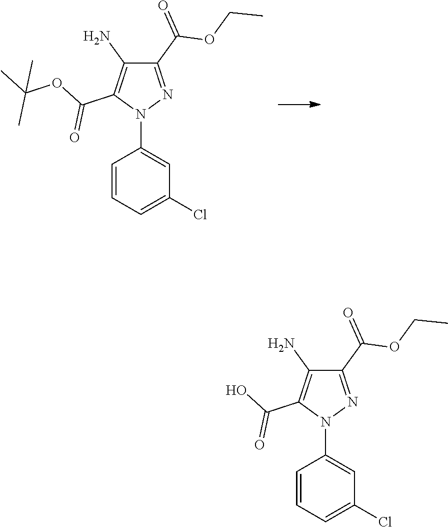 [SMILES: CCOC(=O)c1nn(-c2cccc(Cl)c2)c(C(=O)OC(C)(C)C)c1N ; CCOC(=O)c1nn(-c2cccc(Cl)c2)c(C(=O)O)c1N]

4M HCl in 1,4-dioxane (25 mL, 100 mmol) was added to a solution of 0-5-tert-butyl 0-3-ethyl 4-amino-1-(3-chlorophenyl) pyrazole-3,5-dicarboxylate (2.0 g, 4.9 mmol) in 1,4-dioxane (10 mL). The mixture was stirred at rt for 48 h. The solvent was evaporated to afford the product (1.55 g, 84% yield).

[M+H]⁺=310.0/312.0

¹H NMR (DMSO-d6, 400 MHZ) δ 1.27-1.32 (3H, m), 4.25-4.39 (2H, m), 7.40-7.54 (2H, m), 7.50-7.65 (2H, m)

### tert-Butyl 7-[[4-amino-2-(3-chlorophenyl)-5-ethoxycarbonyl-pyrazole-3-carbonyl]amino]-3,4-dihydro-1H-isoquinoline-2-carboxylate

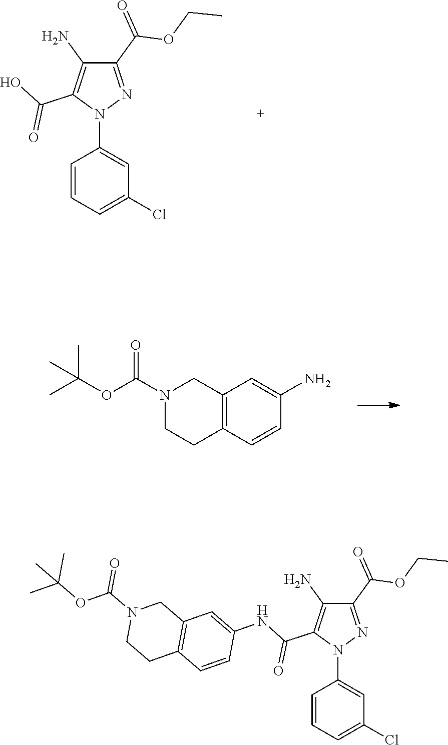 [SMILES: CCOC(=O)c1nn(-c2cccc(Cl)c2)c(C(=O)O)c1N ; CC(C)(C)OC(=O)N1CCc2ccc(N)cc2C1 ; CCOC(=O)c1nn(-c2cccc(Cl)c2)c(C(=O)Nc2ccc3c(c2)CN(C(=O)OC(C)(C)C)CC3)c1N]

O-(7-Azabenzotriazol-1-yl)-N,N,N′,N′-tetramethyluronium hexafluorophosphate (2.12 g, 5.29 mmol) was added to a solution of 4-amino-2-(3-chlorophenyl)-5-ethoxycarbonyl-pyrazole-3-carboxylic acid hydrochloride (1.55 g, 4.07 mmol), 2-boc-7-amino-1,2,3,4-tetrahydroisoquinoline (1.11 g, 4.48 mmol) and DIPEA (2.8 mL, 16.3 mmol) in DMF (25 mL). The mixture was stirred at rt for 20 h. Sat. K₂CO₃ (aq)(150 mL) was added and the mixture extracted with EtOAc (2×100 mL). The combined organic extracts were washed with brine (3×150 mL), dried (Na₂SO₄), filtered and evaporated. Flash chromatography (silica, 0 -100% EtOAc in iso-hexane) afforded the product (1.2 g, 52% yield).

[M+H]⁺=538.3/540.3

¹H NMR (CDCl₃, 400 MHZ) 8:1.32 (3H, t, J=7.0 Hz), 1.42 (9H, s), 2.71 (2H, t, J=5.8 Hz), 3.54 (2H, s), 4.35 (2H, q, J=7.1 Hz), 4.44 (2H, s), 5.33 (2H, s), 7.09 (1H, d, J=8.3 Hz), 7.21-7.25 (1H, m), 7.36-7.44 (2H, m), 7.46-7.56 (3H, m), 10.01 (1H, s)

### tert-Butyl 7-[1-(3-chlorophenyl)-3-ethoxycarbonyl-5-methyl-7-oxo-pyrazolo[4,3-d]pyrimidin-6-yl]-3,4-dihydro-1H-isoquinoline-2-carboxylate

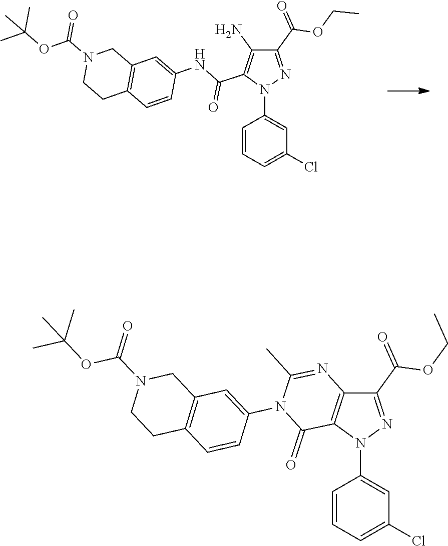

Triethyl orthoacetate (2.0 mL, 10.7 mmol) was added to a solution of tert-butyl7-[[4-amino-2-(3-chlorophenyl)-5-ethoxycarbonyl-pyrazole-3-carbonyl]amino]-3,4-dihydro-1H-isoquinoline-2-carboxylate (1.24 g, 2.14 mmol) in AcOH (10 mL). The mixture was heated to 100° C. for 1 h, cooled to rt and sat. K₂CO₃ (aq)(150 mL) was added. The mixture was extracted with DCM (3×25 mL) and the combined organic extracts were washed with brine (3×150 mL), dried (Na₂SO₄), filtered and evaporated. Flash chromatography (silica, 0-100% EtOAc in iso-hexane) afforded the product (969 mg, 78% yield).

[M+H]⁺=564.2/566.2

¹H NMR (CDCl₃) 1.45-1.51 (12H, m), 2.36 (3H, s), 2.88-2.92 (2H, m), 3.65-3.78 (2H, m), 4.52-4.78 (4H, m), 6.96-7.05 (2H, m), 7.30-7.42 (3H, m), 7.60-7.70 (1H, m), 7.76-7.81 (1H, m)

### 6-(2-tert-Butoxycarbonyl-3,4-dihydro-1H-isoquinolin-7-yl)-1-(3-chlorophenyl)-5-methyl-7-oxo-pyrazolo[4,3-d]pyrimidine-3-carboxylic acid

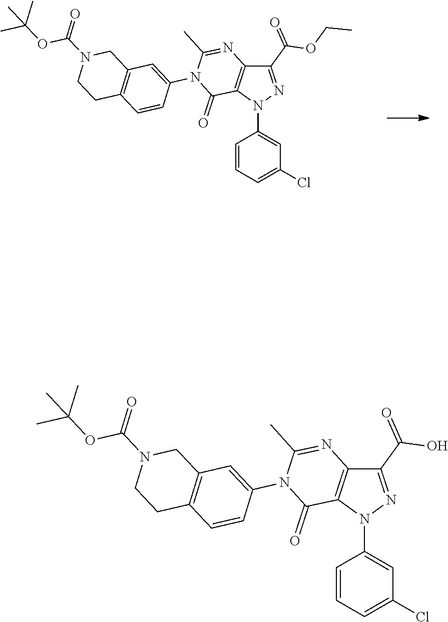

LiOH·H₂O (105 mg, 2.50 mmol) was added to a solution of tert-butyl-7-[1-(3-chlorophenyl)-3-ethoxycarbonyl-5-methyl-7-oxo-pyrazolo[4,3-d]pyrimidin-6-yl]-3,4-dihydro-1H-isoquinoline-2-carboxylate (969 mg, 1.66 mmol) in a mixture of THF (12 mL) and water (3 mL). After 2 h at rt the solvent was evaporated, 5% citric acid (aq)(25 mL) was added and extracted into EtOAc (3×10 mL). The combined organic extracts were washed with brine (3×150 mL), dried (Na₂SO₄), filtered and evaporated to afford the product (961 mg, 100% yield).

[M+H]⁺=536.1/538.1

¹H NMR (DMSO-d6, 400 MHZ) δ 1.43 (9H, s), 2.19 (3H, s), 2.86 (2H, t, J=6.0 Hz), 3.52-3.67 (2H, m), 4.51 -4.60 (2H, m), 7.18-7.26 (2H, m), 7.36 (1H, d, J=8.1 Hz), 7.51-7.60 (2H, m), 7.64-7.74 (1H, m), 7.79-7.84 (1H, m), 13.39 (1H, s)

### tert-Butyl 7-[1-(3-chlorophenyl)-3-[(1,4-dimethylpyrazol-3-yl)methylcarbamoyl]-5-methyl-7-oxo-pyrazolo[4,3-d]pyrimidin-6-yl]-3,4-dihydro-1H-isoquinoline-2-carboxylate

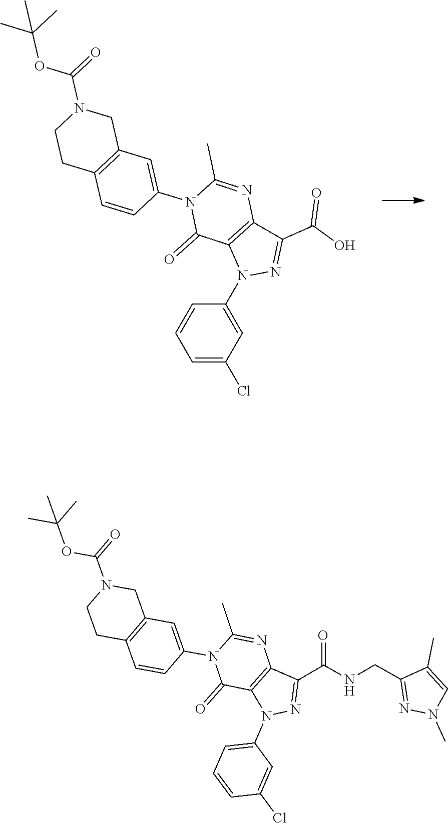 [SMILES: Cc1nc2c(C(=O)O)nn(-c3cccc(Cl)c3)c2c(=O)n1-c1ccc2c(c1)CN(C(=O)OC(C)(C)C)CC2 ; Cc1cn(C)nc1CNC(=O)c1nn(-c2cccc(Cl)c2)c2c(=O)n(-c3ccc4c(c3)CN(C(=O)OC(C)(C)C)CC4)c(C)nc12]

TCFH (117 mg, 0.42 mmol) was added to a solution of 6-(2-tert-butoxycarbonyl-3,4-dihydro-1H-isoquinolin-7-yl)-1-(3-chlorophenyl)-5-methyl-7-oxo-pyrazolo[4,3-d]pyrimidine-3-carboxylic acid (160 mg, 0.28 mmol), 1-(1,4-dimethyl-1H-pyrazol-3-yl) methanamine dihydrochloride (72 mg, 0.36 mmol) and 1-methylimidazole (0.11 mL, 1.40 mmol) in MeCN (2.5 mL) and stirred at rt for 2 h. The solvent was evaporated, the residue dissolved in EtOAc (100 ml), washed with sat. NaHCO₃(aq)(1×50 ml), brine (3× 50 ml), dried (Na₂SO₄), filtered and evaporated. Flash chromatography (silica, 0-100% EtOAc in iso-hexane) afforded the product (154 mg, 81% yield).

[M+H]⁺=643.3/645.3

¹H NMR (CDCl₃, 400 MHZ) δ 1.49 (9H, s), 2.15 (3H, s), 2.30-2.37 (3H, m), 2.88-2.93 (2H, m), 3.35-3.83 (2H, m), 3.89 (3H, s), 4.52-4.73 (2H, m), 4.77 (2H, d, J=5.1 Hz), 6.95-7.05 (2H, m), 7.17 (1H, s), 7.29-7.38 (3H, m), 7.66-7.76 (1H, m), 7.83-7.89 (1H, m), 8.76-8.83 (1H, m)

### 1-(3-Chlorophenyl)-N-[(1,4-dimethylpyrazol-3-yl)methyl]-5-methyl-7-oxo-6-(1,2,3,4-tetrahydroisoquinolin-7-yl) pyrazolo[4,3-d]pyrimidine-3-carboxamide dihydrochloride

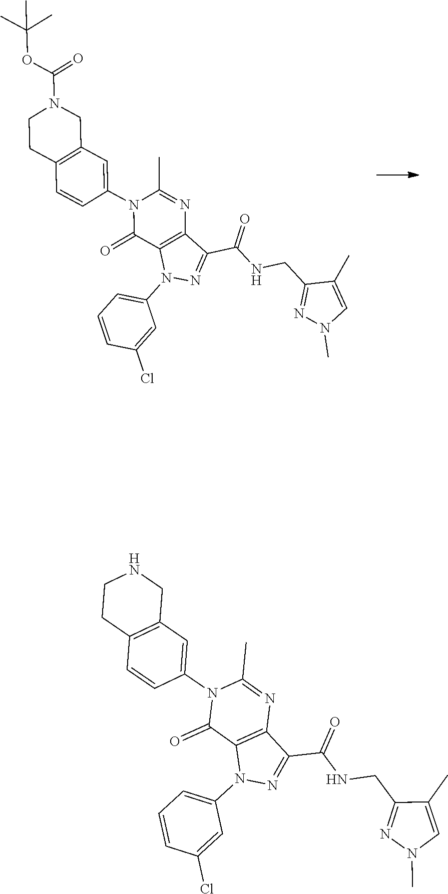 [SMILES: Cc1cn(C)nc1CNC(=O)c1nn(-c2cccc(Cl)c2)c2c(=O)n(-c3ccc4c(c3)CN(C(=O)OC(C)(C)C)CC4)c(C)nc12 ; Cc1cn(C)nc1CNC(=O)c1nn(-c2cccc(Cl)c2)c2c(=O)n(-c3ccc4c(c3)CNCC4)c(C)nc12]

4M HCl in 1,4-dioxane (1.0 mL, 4.0 mmol) was added to a solution of tert-butyl 7-[1-(3-chlorophenyl)-3-[(1,4-dimethylpyrazol-3-yl)methylcarbamoyl]-5-methyl-7-oxo-pyrazolo[4,3-d]pyrimidin-6-yl]-3,4-dihydro-1H-isoquinoline-2-carboxylate (154 mg, 0.23 mmol) in 1,4-dioxane (1 mL). The mixture was stirred at rt for 3 h. The solvent was evaporated to afford the product (199 mg, 100% yield).

[M+H]⁺=543.2/545.2

¹H NMR (DMSO-d6, 400 MHZ) δ 2.01-2.06 (3H, m), 2.20 (3H, s), 3.09 (2H, t, J=6.3 Hz), 3.41-3.45 (2H, m), 3.72-3.76 (3H, m), 4.30-4.35 (2H, m), 4.52 (2H, d, J=5.4 Hz), 7.28-7.36 (2H, m), 7.41-7.51 (2H, m), 7.51-7.61 (2H, m), 7.68-7.77 (1H, m), 7.84-7.89 (1H, m), 8.90 (1H, t, J=5.4 Hz), 9.21-9.29 (3H, m)

### 1-(3-Chlorophenyl)-N-[(1,4-dimethylpyrazol-3-yl)methyl]-6-[2-(2-hydroxyethyl)-3,4-dihydro-1H-isoquinolin-7-yl]-5-methyl-7-oxo-pyrazolo[4,3-d]pyrimidine-3-carboxamide

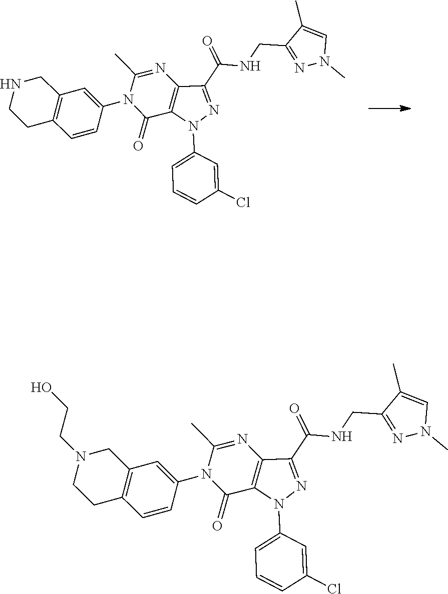 [SMILES: Cc1cn(C)nc1CNC(=O)c1nn(-c2cccc(Cl)c2)c2c(=O)n(-c3ccc4c(c3)CNCC4)c(C)nc12 ; Cc1cn(C)nc1CNC(=O)c1nn(-c2cccc(Cl)c2)c2c(=O)n(-c3ccc4c(c3)CN(CCO)CC4)c(C)nc12]

2-Bromoethanol (0.024 mL, 0.34 mmol) was added to a solution of 1-(3-chlorophenyl)-N-[(1,4-dimethylpyrazol-3-yl)methyl]-5-methyl-7-oxo-6-(1,2,3,4-tetrahydroisoquinolin-7-yl) pyrazolo[4,3-d] pyrimidine-3-carboxamide dihydrochloride (100 mg, 0.11 mmol) and TEA (0.079 mL, 0.57 mmol) in DMF (1 mL). The mixture was stirred at rt for 6 h. Further 2-bromoethanol (0.012 mL, 0.17 mmol) was added and the resulting mixture was stirred at rt for a further 16 h. Sat. K₂CO₃ (aq)(10 mL) was added and the mixture extracted with EtOAc (2×100 mL). The combined organic extracts were washed with brine (3×150 mL), dried (Na₂SO₄), filtered and evaporated. Preparative HPLC (30-60% MeCN containing 0.1% NH₃, in 0.01M NH₄HCO₃ (aq)) afforded the product (22 mg, 33% yield).

[M+H]⁺=587.3/589.2

¹H NMR (DMSO-d6, 400 MHZ) δ 2.00-2.05 (3H, m), 2.20 (3H, s), 2.57 (2H, t, J=6.2 Hz), 2.66-2.83 (2H, m), 2.84-2.90 (2H, m), 3.53-3.66 (4H, m), 3.74 (3H, s), 4.47-4.54 (3H, m), 7.10 (1H, d, J=2.1 Hz), 7.16 (1H, dd, J=8.1, 2.2 Hz), 7.28 (1H, d, J=8.2 Hz), 7.43 (1H, d, J=0.9 Hz), 7.51-7.60 (2H, m), 7.68-7.77 (1H, m), 7.83-7.89 (1H, m), 8.91 (1H, t, J=5.3 Hz)

### Example Number 1238

### 1-(3-Chlorophenyl)-N-[(1,5-dimethylpyrazol-3-yl)methyl]-6-(2-methyl-1-oxo-4,5-dihydro-3H-2-benzazepin-8-yl)-7-oxo-4,5-dihydropyrazolo[3,4-c]pyridine-3-carboxamide

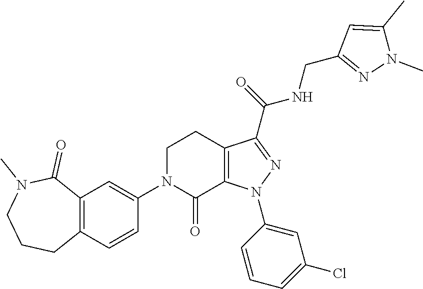

### 7-Bromotetralin-1-one oxime

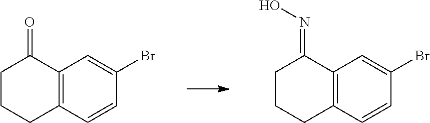 [SMILES: O=C1CCCc2ccc(Br)cc21 ; O/N=C1\CCCc2ccc(Br)cc21]

A solution of 7-bromo-1-tetralone (1.0 g, 4.44 mmol), KOAc (654 mg, 6.66 mmol) and NH₂OH·HCl (370 mg, 5.33 mmol) in EtOH (20 mL) was heated at 80° C. for 2 h and evaporated. The residue was taken up in EtOAc (100 mL), washed with water (100 mL) and brine (100 mL), dried (Na₂SO₄) and evaporated to afford the product (887 mg, 81% yield).

[M+H]⁺=240.0/242.0

¹H NMR (DMSO-d6, 400 MHZ) δ 1.68-1.78 (2H, m), 2.57-2.71 (4H, m), 7.16 (1H, d, J=8.2 Hz), 7.42 (1H, dd, J=8.2, 2.2 Hz), 7.91-7.95 (1H, m), 11.30 (1H, s)

### 8-Bromo-2,3,4,5-tetrahydro-2-benzazepin-1-one

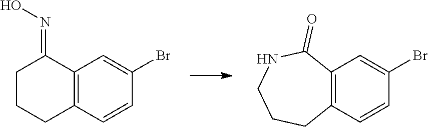 [SMILES: O/N=C1\CCCc2ccc(Br)cc21 ; O=C1NCCCc2ccc(Br)cc21]

A solution of 7-bromotetralin-1-one oxime (880 mg, 3.67 mmol) in SOCI2 (70 mL, 970 mmol) was stirred at 50° C. for 2 h. The mixture was evaporated, the residue was cooled in an ice/water bath and sat. NaHCO₃(aq)(50 mL) was added slowly. The suspension was extracted with EtOAc (100 mL), dried (Na₂SO₄) and evaporated. Flash chromatography (silica, 0-100% EtOAc in isohexane) afforded the product (447 mg, 51% yield).

[M+H]⁺=240.0/242.0

¹H NMR (DMSO-d6, 400 MHZ) δ 1.78-1.94 (2H, m), 2.71 (2H, t, J=7.1 Hz), 2.80-2.96 (2H, m), 7.22-7.27 (1H, m), 7.58-7.61 (1H, m), 7.62 (1H, d, J=2.2 Hz), 8.19 (1H, t, J=6.0 Hz)

### 8-Bromo-2-methyl-4,5-dihydro-3H-2-benzazepin-1-one

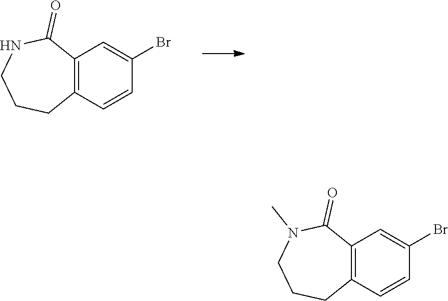

To a solution of 8-bromo-2,3,4,5-tetrahydro-2-benzazepin-1-one (432 mg, 1.80 mmol) in THF (10 mL), were added NaH (60% in mineral oil dispersion, 86 mg, 2.16 mmol) and Mel (0.11 mL, 1.80 mmol). After 20 h at rt, sat. NH₄Cl (aq)(20 mL) was added and extracted with EtOAc (2×40 mL). The combined organic extracts were washed with brine (40 mL), dried (Na₂SO₄) and evaporated. Flash chromatography (silica, 0-10% MeOH in DCM) afforded the product (447 mg, 91% yield).

[M+H]⁺=254.0/256.0

¹H NMR (DMSO-d6, 400 MHZ) δ 1.93-2.02 (2H, m), 2.66 (2H, t, J=7.1 Hz), 3.05 (3H, s), 3.15 (2H, t, J=6.4 Hz), 7.21 (1H, m), 7.56-7.62 (2H, m)

### Ethyl 1-(3-chlorophenyl)-6-(2-methyl-1-oxo-4,5-dihydro-3H-2-benzazepin-8-yl)-7-oxo-4,5-dihydropyrazolo[3,4-c]pyridine-3-carboxylate

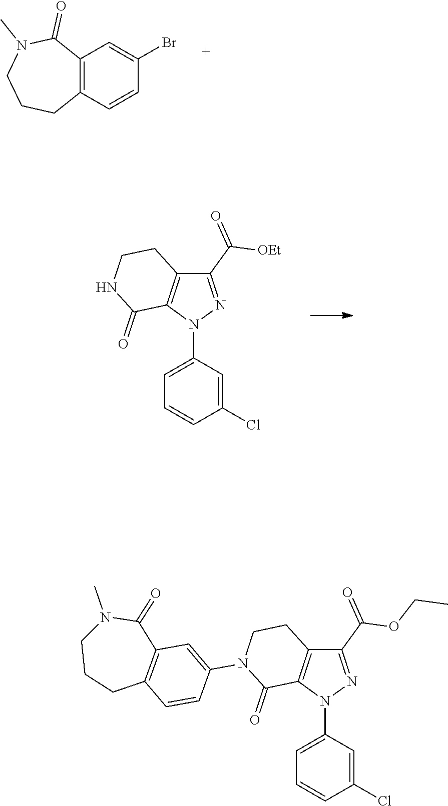 [SMILES: CN1CCCc2ccc(Br)cc2C1=O ; CCOC(=O)c1nn(-c2cccc(Cl)c2)c2c1CCNC2=O ; CCOC(=O)c1nn(-c2cccc(Cl)c2)c2c1CCN(c1ccc3c(c1)C(=O)N(C)CCC3)C2=O]

Cu(I)I (91 mg, 0.48 mmol) was added to a suspension of ethyl 1-(3-chlorophenyl)-7-oxo-5,6-dihydro-4H-pyrazolo[3,4-c]pyridine-3-carboxylate (365 mg, 1.14 mmol), 8-bromo-2-methyl-4,5-dihydro-3H-2-benzazepin-1-one (260 mg, 0.95 mmol), (1S,2S)—N,N′-dimethyl-1,2-cyclohexane-diamine (81 mg, 0.57 mmol) and tripotassium phosphate (505 mg, 2.38 mmol) in 1,4-dioxane (3 mL) at rt. The mixture was heated in a sealed tube at 110° C. for 18 h. The mixture was allowed to cool to rt, filtered through Celite® and washed EtOAc (5×15 mL). The filtrates were washed with water (20 mL) and brine (20 mL), dried (Na₂SO₄) and evaporated. Flash chromatography (silica, 0-100% MeOH: EtOAc (9:1) in iso-hexane) afforded the product (195 mg, 36% yield).

[M+H]⁺=493.2

¹H NMR (methanol-d4, 400 MHz) δ 1.42 (3H, t, J=7.1 Hz), 2.05-2.18 (2H, m), 2.79 (2H, t, J=7.1 Hz), 3.17 (3H, s), 3.27-3.29 (2H, m), 3.32-3.35 (2H, m), 4.17 (2H, t, J=6.8 Hz), 4.43 (2H, q, J=7.1 Hz), 7.29 (1H, d, J=8.1 Hz), 7.40-7.50 (3H, m), 7.53-7.60 (2H, m), 7.68-7.72 (1H, m)

### 1-(3-Chlorophenyl)-6-(2-methyl-1-oxo-4,5-dihydro-3H-2-benzazepin-8-yl)-7-oxo-4,5-dihydropyrazolo[3,4-c]pyridine-3-carboxylic acid

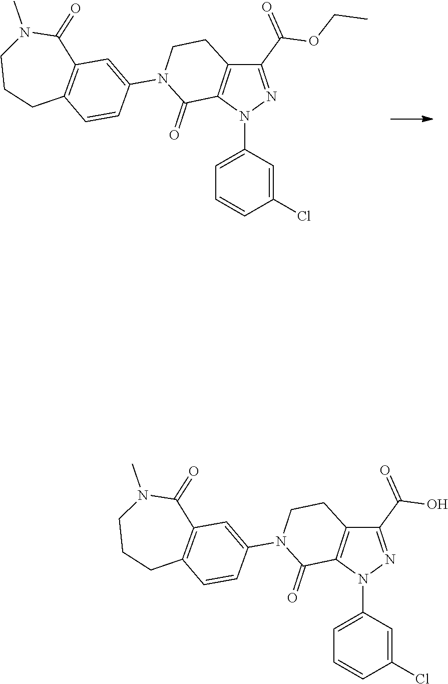 [SMILES: CCOC(=O)c1nn(-c2cccc(Cl)c2)c2c1CCN(c1ccc3c(c1)C(=O)N(C)CCC3)C2=O ; CN1CCCc2ccc(N3CCc4c(C(=O)O)nn(-c5cccc(Cl)c5)c4C3=O)cc2C1=O]

LiOH·H₂O (29 mg, 0.69 mmol) was added to a solution of 1-(3-chlorophenyl)-6-(2-methyl-1-oxo-4,5-dihydro-3H-2-benzazepin-8-yl)-7-oxo-4,5-dihydropyrazolo[3,4-c]pyridine-3-carboxylate (195 mg, 0.34 mmol) in a mixture of THF (2 mL) and water (1 mL). After 2 h at rt, 1M HCl (aq)(1 mL) was added and the solvent evaporated to afford the product (179 mg).

[M+H]⁺=465.2/467.2

¹H NMR (DMSO-d6 400 MHZ) δ 1.91-2.04 (2H, m), 2.63-2.72 (2H, m), 3.05 (3H, s), 3.12-3.23 (4H, m), 4.08 (2H, t, J=6.6 Hz), 7.19-7.30 (1H, m), 7.39 (1H, dd, J=8.1, 2.3 Hz), 7.45-7.49 (1H, m), 7.49-7.56 (2H, m), 7.57-7.62 (1H, m), 7.73-7.77 (1H, m), 13.33 (1H, br. s).

### 1-(3-Chlorophenyl)-N-[(1,5-dimethylpyrazol-3-yl)methyl]-6-(2-methyl-1-oxo-4,5-dihydro-3H-2-benzazepin-8-yl)-7-oxo-4,5-dihydropyrazolo[3,4-c]pyridine-3-carboxamide

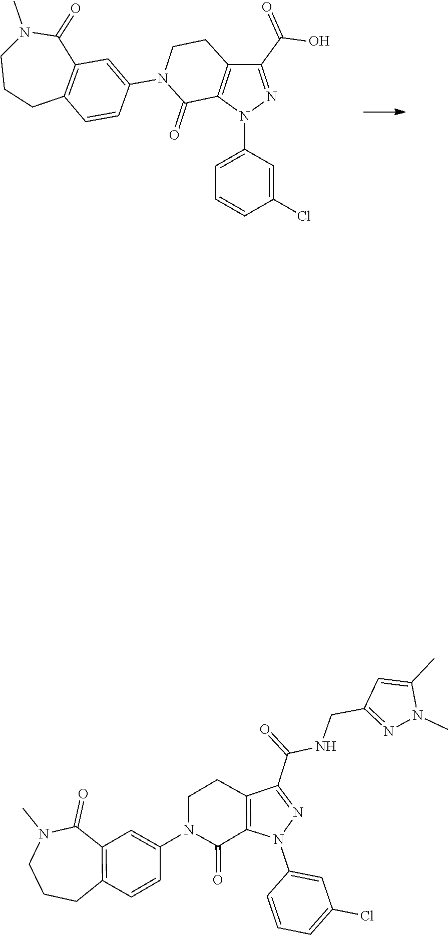

To a solution of 1-(3-chlorophenyl)-6-(2-methyl-1-oxo-4,5-dihydro-3H-2-benzazepin-8-yl)-7-oxo-4,5-dihydropyrazolo[3,4-c]pyridine-3-carboxylic acid (60 mg, 0.11 mmol) in DMF (1.3 mL) was added (1,5-dimethyl-1H-pyrazol-3-yl) methanamine (0.013 mL, 0.11 mmol), DIPEA (0.099 mL, 0.57 mmol) and COMU (73 mg, 0.17 mmol). After 20 h at rt, EtOAc (25 mL) was added and the solution was washed with sat. NaHCO₃(aq)(1×20 mL) and brine (3×50 mL), dried (Na₂SO₄), filtered and evaporated. Purification by preparative HPLC (C18, 42.0-46.4% [MeCN+0.1% NH₃] in NH₄CO₃ in H₂O+0.1% NH₃]) afforded the product (6.0 mg, 9% yield).

[M+H]⁺=572.3/574.2

¹H NMR (DMSO-d6, 400 MHZ) δ 1.93-2.02 (2H, m), 2.19 (3H, d, J=0.7 Hz), 2.69 (2H, t, J=7.1 Hz), 3.05 (3H, s), 3.16 (2H, t, J=6.3 Hz), 3.21 (2H, t, J=6.6 Hz), 3.64 (3H, s), 4.07 (2H, t, J=6.6 Hz), 4.31 (2H, d, J=6.0 Hz), 5.93 (1H, d, J=0.8 Hz), 7.26 (1H, d, J=8.2 Hz), 7.39 (1H, dd, J=8.1, 2.4 Hz), 7.48 (1H, d, J=2.2 Hz), 7.49-7.54 (2H, m), 7.61-7.67 (1H, m), 7.76-7.82 (1H, m), 8.68 (1H, t, J=6.1 Hz)

### Example Number 4002

### 2-[3-(3-Chlorophenyl)-5-[2-(2-hydroxyethyl)-3,4-dihydro-1H-isoquinolin-7-yl]-4-oxo-6,7-dihydropyrazolo[4,3-c]pyridin-1-yl]-N-(cyclopropylmethyl) acetamide

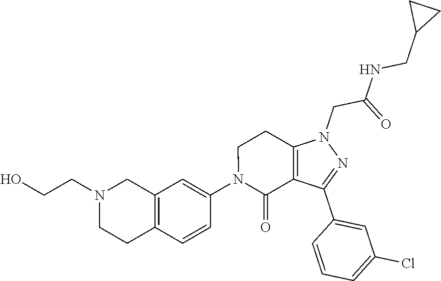 [SMILES: O=C(Cn1nc(-c2cccc(Cl)c2)c2c1CCN(c1ccc3c(c1)CN(CCO)CC3)C2=O)NCC1CC1]

### 3-(Dimethylaminomethylene)piperidine-2,4-dione

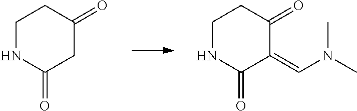

2,4-Piperidinedione (3.10 g, 27.4 mmol) in DMF (90 mL) was treated with N,N-dimethylformamide dimethyl acetal (3.59 g, 30.1 mmol) and heated at 90° C. for 18 h. The mixture was evaporated to afford the product (5.72 g, 100% yield).

[M+H]⁺=169.1

¹H NMR (DMSO-d6, 400 MHZ) δ 2.27-2.32 (2H, m), 3.05 (3H, d, J=0.6 Hz), 3.13-3.19 (2H, m), 3.29 (3H, d, J=0.5 Hz), 7.15 (1H, s), 7.83 (1H, s) 8 1.68-1.78 (2H, m), 2.57-2.71 (4H, m), 7.16 (1H, d, J=8.2 Hz), 7.42 (1H, dd, J=8.2, 2.2 Hz), 7.91-7.95 (1H, m), 11.30 (1H, s)

### 1,5,6,7-Tetrahydropyrazolo[4,3-c]pyridin-4-one

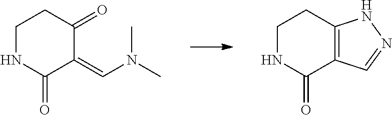

A solution of 3-(dimethylaminomethylene)piperidine-2,4-dione (5.72 g, 24.1 mmol) in EtOH (60 mL) was treated with hydrazine hydrate (1.2 mL, 24.1 mmol) and heated at 100° C. for 18 h. The mixture was evaporated. Chromatography (silica, 0-15% MeOH in DCM) afforded the product (2.57 g, 78% yield).

[M+H]⁺=138.1

¹H NMR (DMSO-d6, 400 MHZ) δ 2.73-2.83 (2H, m), 3.33-3.40 (2H, m), 7.29 (1H, s), 7.63-8.20 (1H, br s), 13.02 (1H, s) 3-Bromo-1,5,6,7-tetrahydropyrazolo[4,3-c]pyridin-4-one

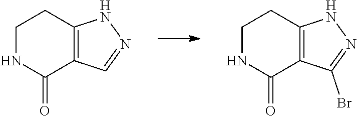 [SMILES: O=C1NCCc2[nH]ncc21 ; O=C1NCCc2[nH]nc(Br)c21]

A solution of 1,5,6,7-tetrahydropyrazolo[4,3-c]pyridin-4-one (2.57 g, 18.7 mmol) in a mixture of EtOH (90 mL) and water (60 mL) was treated with potassium acetate (7.36 g, 75.0 mmol) and Br₂ (I) (1.9 mL, 37.5 mmol) at 0° C. and left stirring for 22 h. Sodium thiosulfate (aq)(50 mL) was added and the mixture filtered through Celite®. The filtrate was washed with water (20 mL) and brine (20 mL), dried (Na₂SO₄) and evaporated. Flash chromatography (silica, 0-15% MeOH in CHCl₃) afforded the product (2.94 g, 73% yield).

[M+H]⁺=216.1/218.1

¹H NMR (DMSO-d6, 400 MHZ) δ 2.86 (2H, t, J=6.8 Hz), 3.36-3.40 (2H, m), 7.34 (1H, s), 13.39 (1H, s)

### 3-Bromo-1-[(4-methoxyphenyl)methyl]-6,7-dihydro-5H-pyrazolo[4,3-c]pyridin-4-one

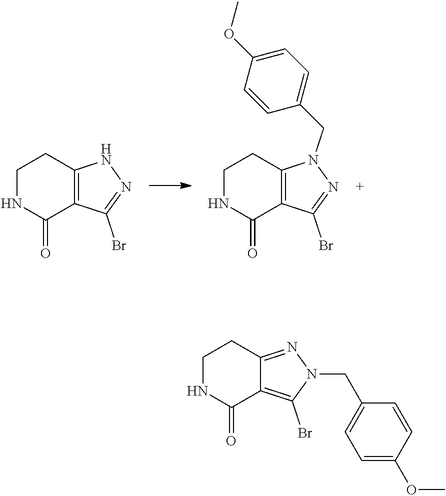 [SMILES: O=C1NCCc2[nH]nc(Br)c21 ; COc1ccc(Cn2nc(Br)c3c2CCNC3=O)cc1 ; COc1ccc(Cn2nc3c(c2Br)C(=O)NCC3)cc1]

A suspension of 3-bromo-1,5,6,7-tetrahydropyrazolo[4,3-c]pyridin-4-one (2.94 g, 13.6 mmol) in acetone (105 mL) was treated with K₂CO₃ (5.64 g, 40.8 mmol) and 4-methoxybenzyl chloride (2.56 g, 16.3 mmol). After 18 h at 60° C. the mixture was diluted with EtOAc (150 mL), washed with water (20 mL) and brine (20 mL), dried (Na₂SO₄) and evaporated to afford a mixture of 3-bromo-1-[(4-methoxyphenyl)methyl]-6,7-dihydro-5H-pyrazolo[4,3-c]pyridin-4-one and 3-bromo-2-[(4-methoxyphenyl)methyl]-6,7-dihydro-5H-pyrazolo[4,3-c]pyridin-4-one (total 2.73 g, 39% yield).

[M+H]⁺=336.0/337.9

### 3-(3-Chlorophenyl)-1-[(4-methoxyphenyl)methyl]-6,7-dihydro-5H-pyrazolo[4,3-c]pyridin-4-one

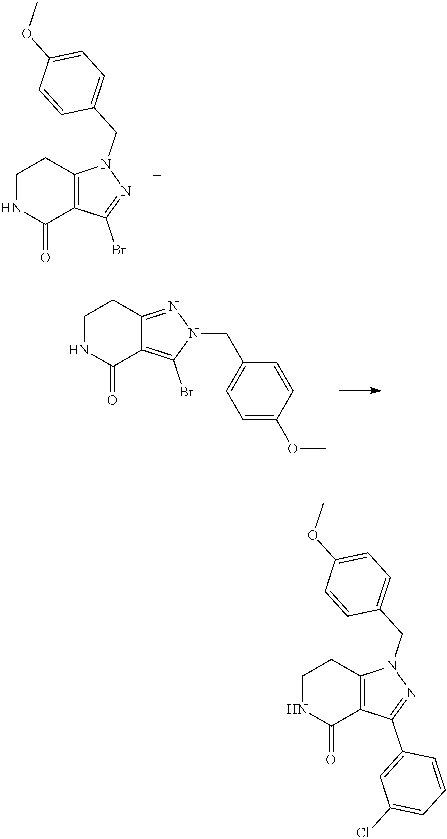 [SMILES: COc1ccc(Cn2nc(Br)c3c2CCNC3=O)cc1 ; COc1ccc(Cn2nc3c(c2Br)C(=O)NCC3)cc1 ; COc1ccc(Cn2nc(-c3cccc(Cl)c3)c3c2CCNC3=O)cc1]

A mixture of 3-bromo-2-[(4-methoxyphenyl)methyl]-6,7-dihydro-5H-pyrazolo[4,3-c]pyridin-4-one and 3-bromo-1-[(4-methoxyphenyl)methyl]-6,7-dihydro-5H-pyrazolo[4,3-c]pyridin-4-one (2.73 g, 7.47 mmol), 3-chlorobenzeneboronic acid (1.40 g, 8.97 mmol) and Na₂CO₃ (2.38 g, 22.4 mmol) were suspended in 1,4-dioxane (120 mL) and water (60 mL), purged with N₂ (g), and tetrakis(triphenylphosphine) palladium (0) (1.73 g, 1.49 mmol) was added. After 18 h at 100° C. the mixture was diluted with EtOAc (150 mL), washed with water (80 mL) and brine (80 mL), dried (Na₂SO₄) and evaporated. Flash chromatography (silica, 0-100% EtOAc in iso-hexane) afforded the product (1.77 g, 43% yield).

[M+H]⁺=368.1/370.1

¹H NMR (DMSO-d6, 400 MHZ) δ 2.92 (2H, t, J=6.8 Hz), 3.37-3.44 (2H, m), 3.72 (3H, s), 5.32 (2H, s), 6.86-6.98 (2H, m), 7.16-7.27 (2H, m), 7.37-7.44 (2H, m), 7.45-7.49 (1H, m), 8.09-8.22 (1H, m), 8.28-8.38 (1H, m)

### tert-Butyl 7-[3-(3-chlorophenyl)-1-[(4-methoxyphenyl)methyl]-4-oxo-6,7-dihydropyrazolo[4,3-c]pyridin-5-yl]-3,4-dihydro-1H-isoquinoline-2-carboxylate

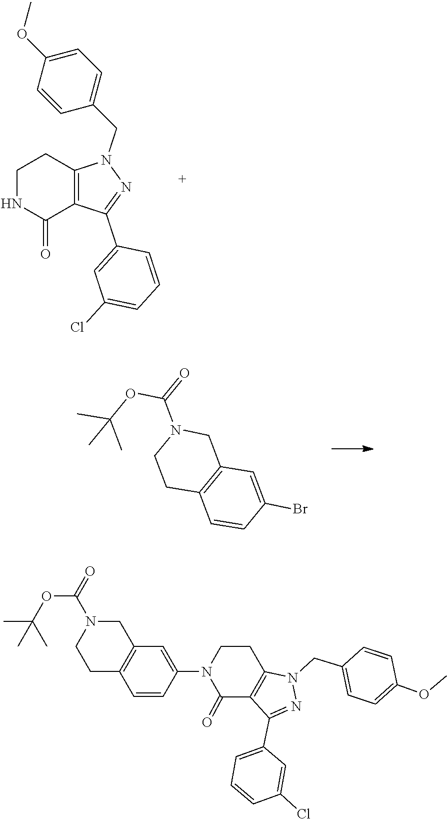 [SMILES: COc1ccc(Cn2nc(-c3cccc(Cl)c3)c3c2CCNC3=O)cc1 ; CC(C)(C)OC(=O)N1CCc2ccc(Br)cc2C1 ; COc1ccc(Cn2nc(-c3cccc(Cl)c3)c3c2CCN(c2ccc4c(c2)CN(C(=O)OC(C)(C)C)CC4)C3=O)cc1]

A solution of 3-(3-chlorophenyl)-1-[(4-methoxyphenyl)methyl]-6,7-dihydro-5H-pyrazolo[4,3-c]pyridin-4-one (1.50 g, 4.08 mmol) and 2-boc-7-bromo-1,2,3,4-tetrahydroisoquinoline (1.53 g, 4.89 mmol) in dry 1,4-dioxane (40 mL) was purged with N₂ (g). Tripotassium phosphate (260 g, 12.2 mmol), (1S,2S)—N,N′-dimethyl-1,2-cyclohexanediamine (464 mg, 3.26 mmol) and Cu(I)I (311 mg, 1.63 mmol) were added sequentially. After heating in a sealed tube at 110° C. for 20 h the mixture was filtered through Celite® and washed with EtOAc (120 mL). The combined filtrates were washed with sat. NaHCO₃(aq)(1×50 mL), brine (3×50 mL), dried (Na₂SO₄), filtered and evaporated. Flash chromatography (silica, 2-60% EtOAc in tert-butyliso-hexane) afforded the product (1.33 g, 54% yield).

[M+H]⁺=599.4/601.4

¹H NMR (DMSO-d6, 400 MHz) δ 1.43 (9H, s), 2.77 (2H, t, J=5.9 Hz), 3.14 (2H, t, J=6.6 Hz), 3.55 (2H, t, J=5.9 Hz), 3.74 (3H, s), 3.97 (2H, t, J=6.7 Hz), 4.46-4.52 (2H, m), 5.37 (2H, s), 6.91-6.97 (2H, m), 7.11-7.19 (3H, m), 7.24-7.31 (2H, m), 7.37-7.42 (2H, m), 8.01-8.09 (1H, m), 8.21-8.26 (1H, m)

### 3-(3-Chlorophenyl)-1-[(4-methoxyphenyl)methyl]-5-(1,2,3,4-tetrahydroisoquinolin-7-yl)-6,7-dihydropyrazolo[4,3-c]pyridin-4-one hydrochloride

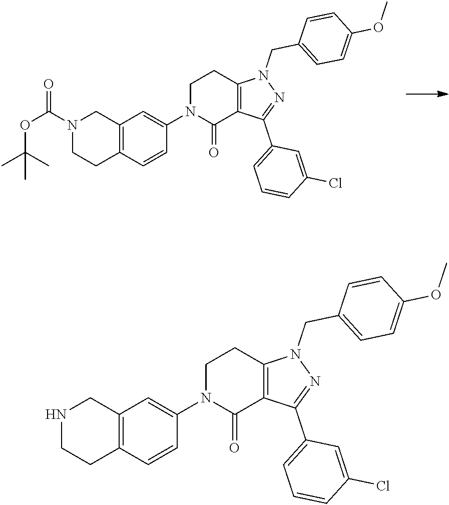 [SMILES: COc1ccc(Cn2nc(-c3cccc(Cl)c3)c3c2CCN(c2ccc4c(c2)CN(C(=O)OC(C)(C)C)CC4)C3=O)cc1 ; COc1ccc(Cn2nc(-c3cccc(Cl)c3)c3c2CCN(c2ccc4c(c2)CNCC4)C3=O)cc1]

To a solution of tert-butyl7-[3-(3-chlorophenyl)-1-[(4-methoxyphenyl)methyl]-4-oxo-6,7-dihydropyrazolo[4,3-c]pyridin-5-yl]-3,4-dihydro-1H-isoquinoline-2-carboxylate (1.33 g, 2.21 mmol) in 1,4-dioxane (5 mL) was added 4M HCl in 1,4-dioxane (15 mL, 60.0 mmol). After 60 h at rt the solvent was evaporated to afford the product (1.15 g, 97% yield).

[M+H]⁺=499.3/501.3

¹H NMR (DMSO-d6, 400 MHZ) δ 3.00 (2H, t, J=6.3 Hz), 3.16 (2H, t, J=6.6 Hz), 3.74 (3H, s), 3.98 (2H, t, J=6.6 Hz), 4.22-4.28 (2H, m), 5.38 (2H, s), 6.90-6.98 (2H, m), 7.19-7.31 (5H, m), 7.37-7.42 (2H, m), 8.00-8.07 (1H, m), 8.22-8.25 (1H, m), 9.29 (2H, br s)

### 3-(3-Chlorophenyl)-5-(1,2,3,4-tetrahydroisoquinolin-7-yl)-6,7-dihydro-1H-pyrazolo[4,3-c]pyridin-4-one

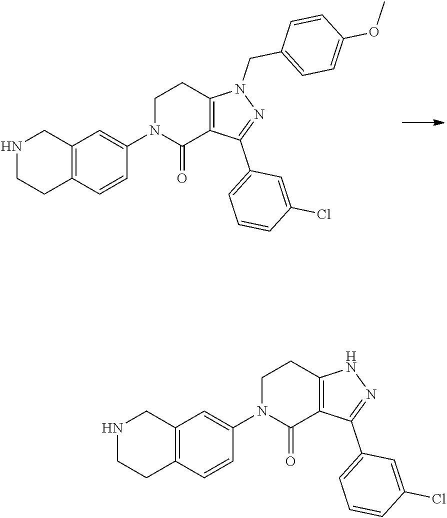 [SMILES: COc1ccc(Cn2nc(-c3cccc(Cl)c3)c3c2CCN(c2ccc4c(c2)CNCC4)C3=O)cc1 ; O=C1c2c(-c3cccc(Cl)c3)n[nH]c2CCN1c1ccc2c(c1)CNCC2]

To a suspension of 3-(3-chlorophenyl)-1-[(4-methoxyphenyl)methyl]-5-(1,2,3,4-tetrahydroisoquinolin-7-yl)-6,7-dihydropyrazolo[4,3-c]pyridin-4-one hydrochloride (1.0 g, 1.87 mmol) in DCE (12 mL) was added TFA (8.0 mL, 104 mmol). After 6 h at 70° C. the solvent was evaporated and the residue dissolved in EtOAc (100 mL). This solution was washed with sat. NaHCO₃(aq)(2×50 mL), brine (3×50 mL), dried (Na₂SO₄), filtered and evaporated to afford the product. (525 mg, 74% yield).

[M+H]⁺=379.2/381.2

¹H NMR (DMSO-d6, 400 MHZ) δ 2.70 (2H, t, J=5.8 Hz), 2.97 (2H, t, J=5.9 Hz), 3.07 (2H, t, J=6.6 Hz), 3.86 (2H, s), 3.95 (2H, t, J=6.6 Hz), 6.92-7.03 (1H, m), 7.03-7.13 (2H, m), 7.37-7.50 (2H, m), 7.98-8.06 (1H, m), 8.22-8.29 (1H, m)

### 5-[2-[2-[tert-Butyl (dimethyl) silyl]oxyethyl]-3,4-dihydro-1H-isoquinolin-7-yl]-3-(3-chlorophenyl)-6,7-dihydro-1H-pyrazolo[4,3-c]pyridin-4-one

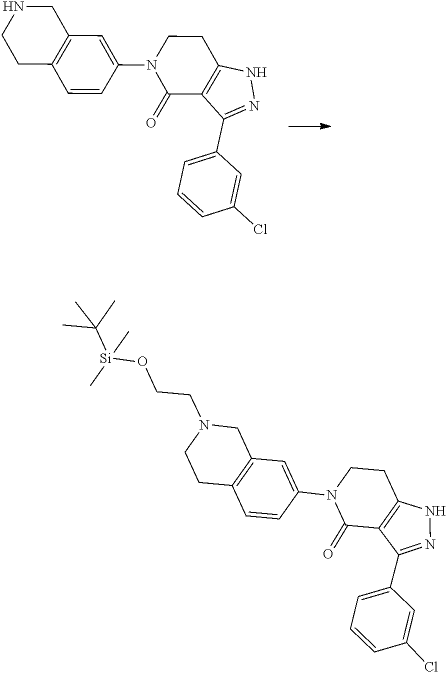 [SMILES: O=C1c2c(-c3cccc(Cl)c3)n[nH]c2CCN1c1ccc2c(c1)CNCC2 ; CC(C)(C)[Si](C)(C)OCCN1CCc2ccc(N3CCc4[nH]nc(-c5cccc(Cl)c5)c4C3=O)cc2C1]

To a solution of 3-(3-chlorophenyl)-5-(1,2,3,4-tetrahydroisoquinolin-7-yl)-6,7-dihydro-1H-pyrazolo[4,3-c]pyridin-4-one (500 mg,1.32 mmol) in DMF (12 mL) were added TEA (0.92 mL, 6.60 mmol) and 2-bromoethoxy-t-butyldimethylsilane (0.57 mL, 2.64 mmol). After 40 h at rt the mixture was diluted with EtOAc (75 mL), washed with sat. NaHCO₃(aq)(2×50 mL), brine (3×50 mL), dried (Na₂SO₄), filtered and evaporated. Flash chromatography (silica, 0-10% MeOH in DCM) afforded the product (263 mg, 37% yield).

[M+H]⁺=537.2/539.1

¹H NMR (DMSO-d6, 400 MHZ) δ 0.05 (6H, s), 0.87 (9H, s), 2.60 (2H, t, J=6.2 Hz), 2.70-2.77 (2H, m), 2.77 -2.83 (2H, m), 2.98-3.19 (H, m), 3.61 (2H, s), 3.77 (2H, t, J=6.1 Hz), 3.95 (2H, t, J=6.4 Hz), 6.99 (1H, s), 7.05-7.17 (2H, m), 7.33-7.46 (1H, m), 7.44-7.52 (1H, m), 7.84-8.11 (1H, m), 8.15-8.35 (1H, m), 13.36-13.62 (1H, m)

### Methyl 2-[5-[2-[2-[tert-butyl(dimethyl) silyl]oxyethyl]-3,4-dihydro-1H-isoquinolin-7-yl]-3-(3-chlorophenyl)-4-oxo-6,7-dihydropyrazolo[4,3-c]pyridin-1-yl]acetate

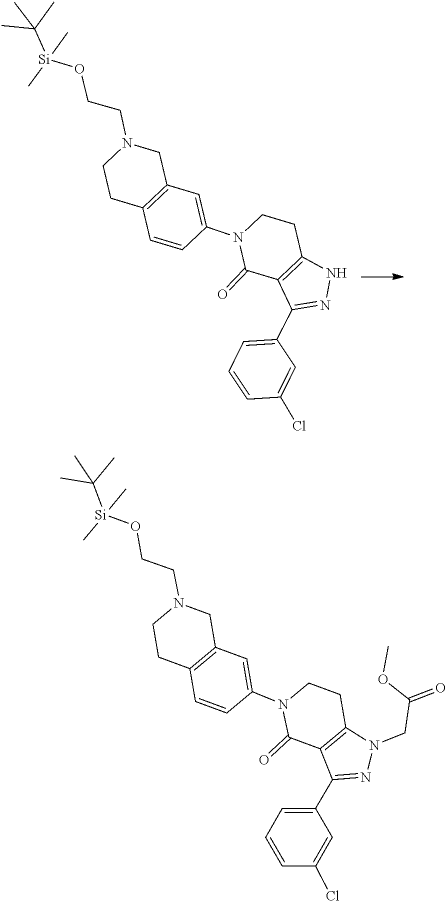 [SMILES: CC(C)(C)[Si](C)(C)OCCN1CCc2ccc(N3CCc4[nH]nc(-c5cccc(Cl)c5)c4C3=O)cc2C1 ; COC(=O)Cn1nc(-c2cccc(Cl)c2)c2c1CCN(c1ccc3c(c1)CN(CCO[Si](C)(C)C(C)(C)C)CC3)C2=O]

To a solution of 5-[2-[2-[tert-butyl(dimethyl) silyl]oxyethyl]-3,4-dihydro-1H-isoquinolin-7-yl]-3-(3-chlorophenyl)-6,7-dihydro-1H-pyrazolo[4,3-c]pyridin-4-one (150 mg, 0.28 mmol) in DMF (3 mL) were added Cs₂CO₃ (136 mg, 0.42 mmol) and 2-bromomethyl acetate (0.034 mL, 0.36 mmol). After 4 h at rt the mixture was diluted with EtOAc (50 mL) and washed with sat. NaHCO₃(aq)(2×20 mL), brine (3×20 mL), dried (Na₂SO₄), filtered and evaporated. Flash chromatography (silica, 0-5% MeOH in DCM) afforded the product (144 mg, 84% yield).

[M+H]⁺=609.2/611.3

¹H NMR (DMSO-d6, 400 MHZ) δ 0.05 (6H, s), 0.87 (9H, s), 2.57-2.64 (2H, m), 2.72-2.76 (2H, m), 2.78-2.81 (2H, m), 3.10 (2H, t, J=6.6 Hz), 3.58-3.64 (2H, m), 3.74 (3H, s), 3.78 (2H, t, J=6.3 Hz), 3.98 (2H, t, J=6.6 Hz), 5.26 (2H, s), 7.02 (1H, s), 7.11 (2H, s), 7.33-7.47 (2H, m), 8.00-8.08 (1H, m), 8.18-8.28 (1H, m)

### 2-[3-(3-Chlorophenyl)-5-[2-(2-hydroxyethyl)-3,4-dihydro-1H-isoquinolin-7-yl]-4-oxo-6,7-dihydropyrazolo[4,3-c]pyridin-1-yl]acetic acid hydrochloride

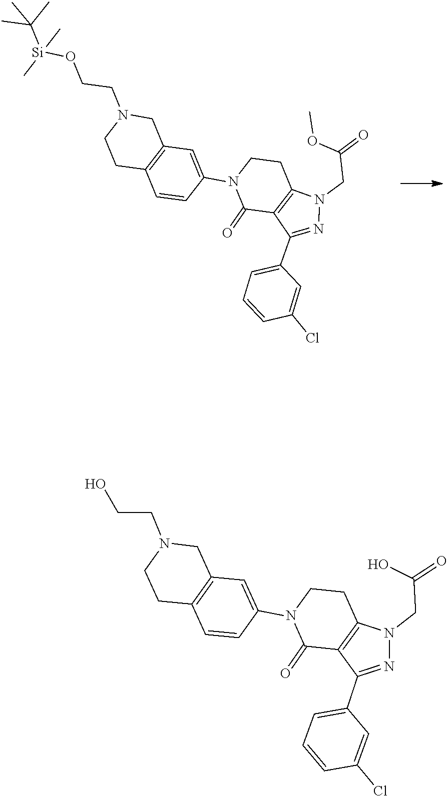 [SMILES: COC(=O)Cn1nc(-c2cccc(Cl)c2)c2c1CCN(c1ccc3c(c1)CN(CCO[Si](C)(C)C(C)(C)C)CC3)C2=O ; O=C(O)Cn1nc(-c2cccc(Cl)c2)c2c1CCN(c1ccc3c(c1)CN(CCO)CC3)C2=O]

To a solution of methyl 2-[5-[2-[2-[tert-butyl(dimethyl) silyl]oxyethyl]-3,4-dihydro-1H-isoquinolin-7-yl]-3-(3-chlorophenyl)-4-oxo-6,7-dihydropyrazolo[4,3-c]pyridin-1-yl]acetate (140 mg, 0.23 mmol) in a mixture of THF (3 mL) and water (1 mL) was added LiOH·H₂O (24 mg, 0.57 mmol). After 20 h at rt 4M HCl in 1,4-dioxane (0.50 mL, 2.00 mmol) was added and after a further 1 h at rt the mixture was evaporated to afford the product (163 mg).

[M+H]⁺=481.1/483.1

### 2-[3-(3-Chlorophenyl)-5-[2-(2-hydroxyethyl)-3,4-dihydro-1H-isoquinolin-7-yl]-4-oxo-6,7-dihydropyrazolo[4,3-c]pyridin-1-yl]-N-(cyclopropylmethyl) acetamide

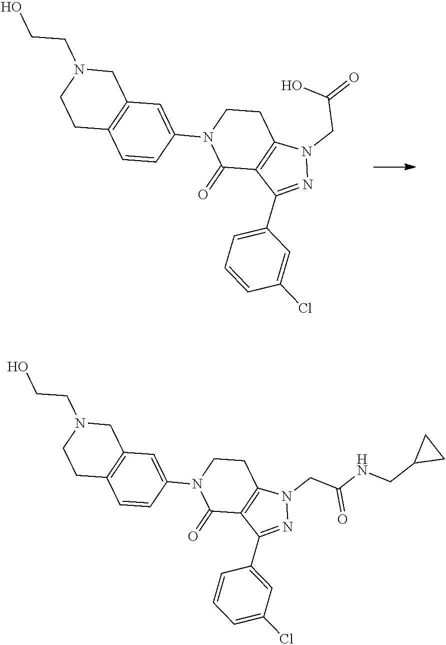 [SMILES: O=C(O)Cn1nc(-c2cccc(Cl)c2)c2c1CCN(c1ccc3c(c1)CN(CCO)CC3)C2=O ; O=C(Cn1nc(-c2cccc(Cl)c2)c2c1CCN(c1ccc3c(c1)CN(CCO)CC3)C2=O)NCC1CC1]

To a solution of 2-[3-(3-chlorophenyl)-5-[2-(2-hydroxyethyl)-3,4-dihydro-1H-isoquinolin-7-yl]-4-oxo-6,7-dihydropyrazolo[4,3-c]pyridin-1-yl]acetic acid hydrochloride (60 mg, 0.12 mmol) in DMF (2 mL) were added aminomethylcyclopropane (0.015 mL,0.17 mmol), DIPEA (0.10 mL, 0.58 mmol) and COMU (74 mg, 0.17 mmol). After 2 h at rt the mixture was diluted with EtOAc (50 mL) and washed with sat. NaHCO₃(aq) (2×15 mL), brine (3×15 mL), dried (Na₂SO₄), filtered and evaporated. Purification by preparative HPLC (C18, 40-57% [MeCN+0.1% NH₃] in [10 mM NH₄CO₃ in H₂O+0.1% NH₃]) afforded the product (24 mg, 37% yield).

[M+H]⁺=534.2/536.1

¹H NMR (DMSO-d6, 400 MHZ) δ 0.15-0.22 (2H, m), 0.40-0.48 (2H, m), 0.88-0.99 (1H, m), 2.54-2.62 (2H, m), 2.69-2.76 (2H, m), 2.77-2.85 (2H, m), 3.00 (2H, dd, J=6.9, 5.5 Hz), 3.11 (2H, t, J=6.7 Hz), 3.55 -3.63 (4H, m), 3.97 (2H, t, J=6.7 Hz), 4.46-4.53 (1H, m), 4.93 (2H, s), 7.04 (1H, s), 7.08-7.13 (2H, m), 7.38-7.43 (2H, m), 7.99-8.06 (1H, m), 8.21-8.25 (1H, m), 8.42 (1H, t, J=5.6 Hz)

### Example Number 1288

### 1-(3-Chlorophenyl)-N-[(1,5-dimethylpyrazol-3-yl)methyl]-6-[4-(2-hydroxyethyl)-5-oxo-2,3-dihydro-1,4-benzoxazepin-7-yl]-7-oxo-4,5-dihydropyrazolo[3,4-c]pyridine-3-carboxamide

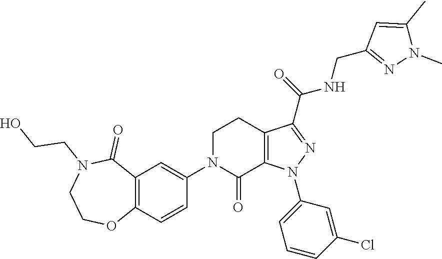 [SMILES: Cc1cc(CNC(=O)c2nn(-c3cccc(Cl)c3)c3c2CCN(c2ccc4c(c2)C(=O)N(CCO)CCO4)C3=O)nn1C]

### Ethyl 5-bromo-2-[2-(tert-butoxycarbonylamino) ethoxy]benzoate

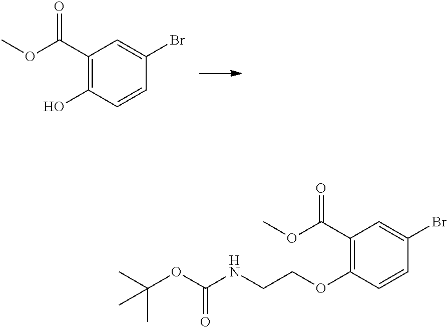 [SMILES: COC(=O)c1cc(Br)ccc1O ; COC(=O)c1cc(Br)ccc1OCCNC(=O)OC(C)(C)C]

To a solution of methyl 5-bromosalicylate (1.0 g, 4.3 mmol) and N-boc-ethanolamine (1.0 mL, 6.5 mmol) in THF (30 mL) at 0° C. were added PPh₃ (1.70 g, 6.49 mmol) and DIAD (1.3 mL, 6.49 mmol). After 3 h at rt the mixture was diluted with EtOAc (100 mL) and washed with sat. NaHCO₃(aq)(1×50 mL), brine (3×50 mL), dried (Na₂SO₄), filtered and evaporated. Flash chromatography (silica, 2-50% EtOAc in iso-hexane) afforded the product (2.13 g, 100% yield).

[M−Boc+H]⁺=273.9/275.9

¹H NMR (DMSO-d6, 400 MHZ) δ 1.37 (9H, s), 3.29 (2H, q, J=5.9 Hz), 3.80 (3H, s), 3.95-4.07 (2H, m), 6.85 (1H, t, J=5.5 Hz), 7.14 (1H, d, J=8.9 Hz), 7.68 (1H, dd, J=8.9, 2.6 Hz), 7.75 (1H, d, J=2.6 Hz)

### 5-Bromo-2-[2-(tert-butoxycarbonylamino) ethoxy]benzoic acid

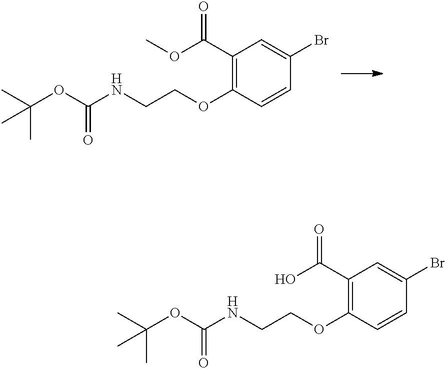 [SMILES: COC(=O)c1cc(Br)ccc1OCCNC(=O)OC(C)(C)C ; CC(C)(C)OC(=O)NCCOc1ccc(Br)cc1C(=O)O]

To a solution of methyl 5-bromo-2-[2-(tert-butoxycarbonylamino) ethoxy]benzoate (1.60 g, 4.28 mmol) in a mixture of THF (30 mL) and water (10 mL) was added LiOH·H₂O (448 mg, 10.7 mmol). After 3 h at rt water (30 mL) was added and the mixture washed with EtOAc (50 mL). The aqueous phase was acidified with 1M HCl (aq) until pH˜2-3 and extracted with EtOAc (2×50 mL). The combined organic extracts were washed with brine (3×50 mL), dried (Na₂SO₄), filtered and evaporated to afford the product (1.22 g, 79% yield).

[M−Boc+H]⁺=259.9/261.9

¹H NMR (DMSO-d6, 400 MHZ) δ 1.37 (9H, s), 3.28 (2H, q, J=6.0 Hz), 4.04 (2H, t, J=6.1 Hz), 6.83 (1H, t, J=5.7 Hz), 7.12 (1H, d, J=8.9 Hz), 7.65 (1H, dd, J=8.9, 2.7 Hz), 7.73 (1H, d, J=2.6 Hz), 12.88 (1H, s br).

### 2-(2-Aminoethoxy)-5-bromo-benzoic acid hydrochloride

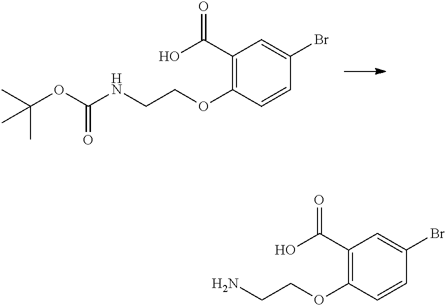 [SMILES: CC(C)(C)OC(=O)NCCOc1ccc(Br)cc1C(=O)O ; NCCOc1ccc(Br)cc1C(=O)O]

To a solution of 5-bromo-2-[2-(tert-butoxycarbonylamino) ethoxy]benzoic acid (1.22 g, 3.39 mmol) in 1,4-dioxane (10 mL) was added 4M HCl in 1,4-dioxane (20 mL, 80.0 mmol). After 20 h at rt, the solvent was evaporated to afford the product (1.01 g, 100% yield)

[M+H]⁺=260.0/262.0

¹H NMR (DMSO-d6, 400 MHz) δ 3.08-3.29 (2H, m), 4.27 (2H, t, J=5.3 Hz), 7.21 (1H, d, J=8.9 Hz), 7.72 (1H, dd, J=8.8, 2.6 Hz), 7.80 (1H, d, J=2.6 Hz), 8.10 (3H, s, br), 13.14 (1H, s, br)

### 7-Bromo-3,4-dihydro-2H-1,4-benzoxazepin-5-one

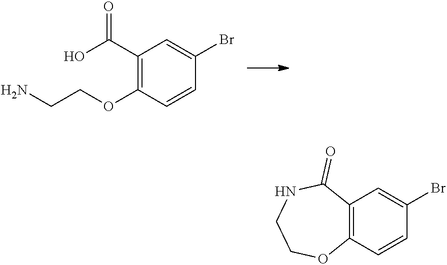 [SMILES: NCCOc1ccc(Br)cc1C(=O)O ; O=C1NCCOc2ccc(Br)cc21]

To a suspension of 2-(2-aminoethoxy)-5-bromo-benzoic acid hydrochloride (1.0 g, 3.37 mmol) in MeCN (150 mL) were added 1-methylimidazole (1.3 mL, 16.9 mmol) and TCFH (1.23 g, 4.38 mmol). After 2 h at rt, water (10 mL) and EtOAc (100 mL) were added. The organic extract was washed with sat. NaHCO₃(aq) (1×50 mL), brine (3×50 mL), dried (Na₂SO₄), filtered and evaporated. Flash chromatography (silica, 0-8% MeOH in DCM) afforded the product (782 mg, 96% yield).

[M+H]⁺=240.0/242.0

¹H NMR (DMSO-d6, 400 MHZ) δ 3.33-3.39 (2H, m), 4.27-4.33 (2H, m), 6.98 (1H, d, J=8.7 Hz), 7.61 (1H, dd, J=8.7, 2.7 Hz), 7.88 (1H, d, J=2.7 Hz), 8.43-8.51 (1H, m)

### 7-Bromo-4-[2-[tert-butyl(dimethyl) silyl]oxyethyl]-2,3-dihydro-1,4-benzoxazepin-5-one

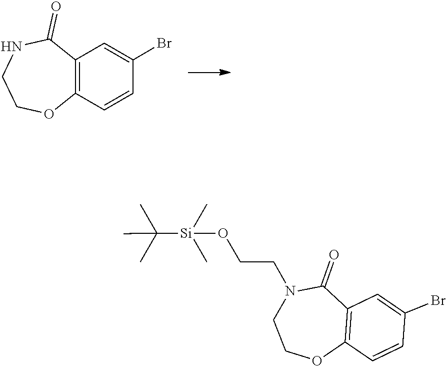 [SMILES: O=C1NCCOc2ccc(Br)cc21 ; CC(C)(C)[Si](C)(C)OCCN1CCOc2ccc(Br)cc2C1=O]

To a solution of 7-bromo-3,4-dihydro-2H-1,4-benzoxazepin-5-one (780 mg, 3.22 mmol) in DMF (20 mL) was added NaH (60% oil dispersion, 168 mg, 4.19 mmol). After 1 h at rt 2-bromoethoxy-t-butyldimethylsilane (0.90 mL, 4.19 mmol) was added dropwise. After a further 2 h sat. NH₄Cl (aq)(25 mL) was added and the mixture extracted with EtOAc (125 mL). The organic extract was washed with brine (3× 50 mL), dried (Na₂SO₄), filtered and evaporated. Flash chromatography (silica, 2-50% EtOAc in iso-hexane) afforded the product (980 mg, 76% yield).

[M+H]⁺=400.1/402.1

¹H NMR (DMSO-d6, 400 MHZ) δ 0.04 (6H, s), 0.85 (9H, s), 3.56-3.66 (4H, m), 3.76 (2H, t, J=5.6 Hz), 4.37 (2H, dd, J=5.4, 4.4 Hz), 6.99 (1H, d, J=8.6 Hz), 7.62 (1H, dd, J=8.6, 2.6 Hz), 7.72 (1H, d, J=2.6 Hz)

### Ethyl 6-[4-[2-[tert-butyl(dimethyl) silyl]oxyethyl]-5-oxo-2,3-dihydro-1,4-benzoxazepin-7-yl]-1-(3-chlorophenyl)-7-oxo-4,5-dihydropyrazolo[3,4-c]pyridine-3-carboxylate

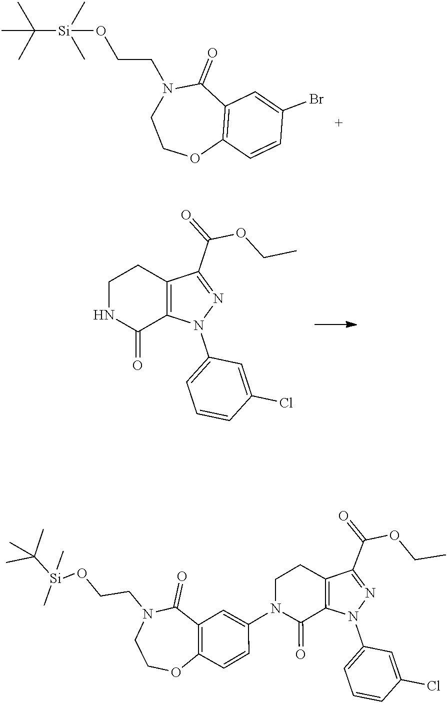 [SMILES: CC(C)(C)[Si](C)(C)OCCN1CCOc2ccc(Br)cc2C1=O ; CCOC(=O)c1nn(-c2cccc(Cl)c2)c2c1CCNC2=O ; CCOC(=O)c1nn(-c2cccc(Cl)c2)c2c1CCN(c1ccc3c(c1)C(=O)N(CCO[Si](C)(C)C(C)(C)C)CCO3)C2=O]

A suspension of ethyl 1-(3-chlorophenyl)-7-oxo-5,6-dihydro-4H-pyrazolo[3,4-c]pyridine-3-carboxylate (335 mg, 1.05 mmol) and 7-bromo-4-[2-[tert-butyl(dimethyl) silyl]oxyethyl]-2,3-dihydro-1,4-benzoxazepin-5-one (350 mg, 0.874 mmol) in 1,4-dioxane (8 mL) and DMF (0.8 mL) was purged with N₂ (g). Tripotassium phosphate (557 mg, 2.62 mmol), (1S,2S)—N,N′-dimethyl-1,2-cyclohexanediamine (50 mg, 0.350 mmol) and Cu(I)I (33 mg, 0.175 mmol) were added sequentially. After heating at 100° C. in a sealed tube for 20 h the mixture was filtered through Celite® and washed with EtOAc (75 mL). The combined filtrates were washed with sat. NaHCO₃(aq)(1×50 mL), brine (3×50 mL), dried (Na₂SO₄), filtered and evaporated. Flash chromatography (silica, 2-60% EtOAc in iso-hexane) afforded the product (416 mg, 74% yield).

[M+H]⁺=639.0/641.0

¹H NMR (DMSO-d6, 400 MHZ) δ 0.04 (6H, s), 0.86 (9H, s), 1.33 (3H, t, J=7.1 Hz), 3.21 (2H, t, J=6.6 Hz), 3.58 (2H, t, J=5.1 Hz), 3.62 (2H, t, J=5.7 Hz), 3.76 (2H, t, J=5.6 Hz), 4.06 (2H, t, J=6.6 Hz), 4.31-4.40 (4H, m), 7.04 (1H, d, J=8.6 Hz), 7.45 (1H, dd, J=8.6, 2.7 Hz), 7.49-7.58 (2H, m), 7.58-7.65 (2H, m), 7.73-7.77 (1H, m)

### 1-(3-Chlorophenyl)-6-[4-(2-hydroxyethyl)-5-oxo-2,3-dihydro-1,4-benzoxazepin-7-yl]-7-oxo-4,5-dihydropyrazolo[3,4-c]pyridine-3-carboxylic acid

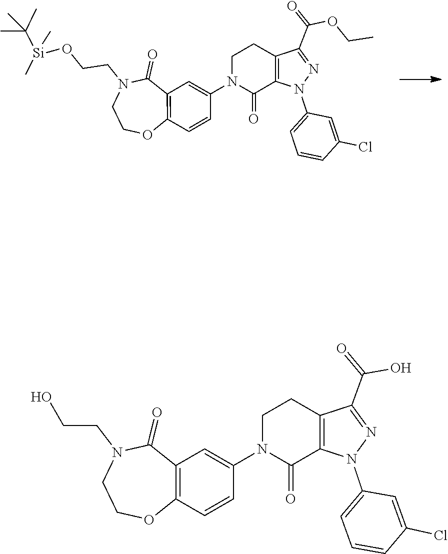 [SMILES: CCOC(=O)c1nn(-c2cccc(Cl)c2)c2c1CCN(c1ccc3c(c1)C(=O)N(CCO[Si](C)(C)C(C)(C)C)CCO3)C2=O ; O=C(O)c1nn(-c2cccc(Cl)c2)c2c1CCN(c1ccc3c(c1)C(=O)N(CCO)CCO3)C2=O]

To a solution of ethyl 6-[4-[2-[tert-butyl(dimethyl) silyl]oxyethyl]-5-oxo-2,3-dihydro-1,4-benzoxazepin-7-yl]-1-(3-chlorophenyl)-7-oxo-4,5-dihydropyrazolo[3,4-c]pyridine-3-carboxylate (416 mg, 0.65 mmol) in THF (6 mL) and water (2 mL) was added LiOH·H₂O (68 mg, 1.63 mmol). After 20 h at rt 4M HCl in 1,4-dioxane (3 mL) was added. After a further 1 h the solvent was evaporated to afford the product (328 mg, 100% yield).

[M+H]⁺496.9/498.8

¹H NMR (DMSO-d6, 400 MHZ) δ 0.04 (6H, s), 0.86 (9H, s), 1.33 (3H, t, J=7.1 Hz), 3.21 (2H, t, J=6.6 Hz), 3.58 (2H, t, J=5.1 Hz), 3.62 (2H, t, J=5.7 Hz), 3.76 (2H, t, J=5.6 Hz), 4.06 (2H, t, J=6.6 Hz), 4.31-4.40 (4H, m), 7.04 (1H, d, J=8.6 Hz), 7.45 (1H, dd, J=8.6, 2.7 Hz), 7.49-7.58 (2H, m), 7.58-7.65 (2H, m), 7.73-7.77 (1H, m)

### 1-(3-Chlorophenyl)-N-[(1,5-dimethylpyrazol-3-yl)methyl]-6-[4-(2-hydroxyethyl)-5-oxo-2,3-dihydro-1,4-benzoxazepin-7-yl]-7-oxo-4,5-dihydropyrazolo[3,4-c]pyridine-3-carboxamide

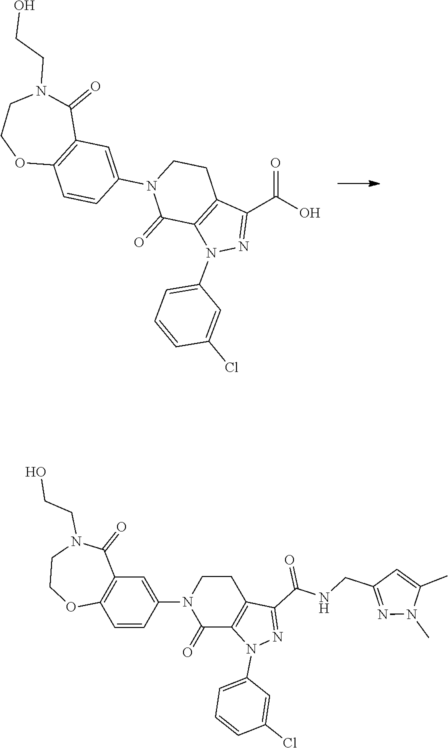 [SMILES: O=C(O)c1nn(-c2cccc(Cl)c2)c2c1CCN(c1ccc3c(c1)C(=O)N(CCO)CCO3)C2=O ; Cc1cc(CNC(=O)c2nn(-c3cccc(Cl)c3)c3c2CCN(c2ccc4c(c2)C(=O)N(CCO)CCO4)C3=O)nn1C]

To a solution of 1-(3-chlorophenyl)-6-[4-(2-hydroxyethyl)-5-oxo-2,3-dihydro-1,4-benzoxazepin-7-yl]-7-oxo-4,5-dihydropyrazolo[3,4-c]pyridine-3-carboxylic acid (60 mg, 0.12 mmol) in DMF (1.2 mL) were added (1,5-dimethyl-1H-pyrazol-3-yl) methanamine (20 μL, 0.18 mmol), DIPEA (0.084 mL, 0.48 mmol) and COMU (78 mg, 0.18 mmol). After 2 h at rt the mixture was diluted with EtOAc (50 mL) and washed with sat. NaHCO₃(aq)(2×15 mL), brine (3×15 mL), dried (Na₂SO₄), filtered and evaporated. Purification by preparative HPLC (C18, 40-57% [MeCN+0.1% NH₃] in [10 mM NH₄CO₃ in H₂O+0.1% NH₃]) afforded the product (50 mg, 68% yield).

[M+H] +603.8/605.8

¹H NMR (DMSO-d6, 400 MHZ) δ 2.19 (3H, s), 3.21 (2H, t, J=6.6 Hz), 3.55-3.62 (6H, m), 3.64 (3H, s), 4.04 (2H, t, J=6.6 Hz), 4.31 (2H, d, J=6.0 Hz), 4.37 (2H, t, J=5.0 Hz), 4.81-4.85 (1H, m), 5.93 (1H, s), 7.04 (1H, d, J=8.6 Hz), 7.44 (1H, dd, J=8.6, 2.7 Hz), 7.47-7.57 (2H, m), 7.57-7.69 (2H, m), 7.80 (1H, d, J=1.1 Hz), 8.68 (1H, t, J=6.1 Hz)

### Example Number 4001

### 1-(3-Chlorophenyl)-N-[(1,5-dimethylpyrazol-3-yl)methyl]-5-[2-(2-hydroxyethyl)-3,4-dihydro-1H-isoquinolin-7-yl]-6-oxo-4H-pyrrolo[3,4-c]pyrazole-3-carboxamide

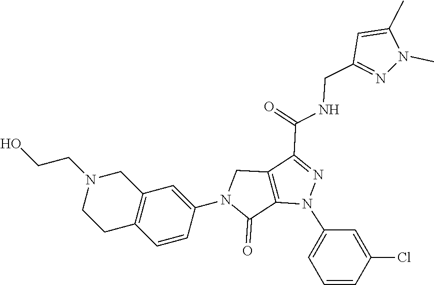 [SMILES: Cc1cc(CNC(=O)c2nn(-c3cccc(Cl)c3)c3c2CN(c2ccc4c(c2)CN(CCO)CC4)C3=O)nn1C]

### O-5-tert-Butyl O-3-ethyl 1H-pyrazole-3,5-dicarboxylate

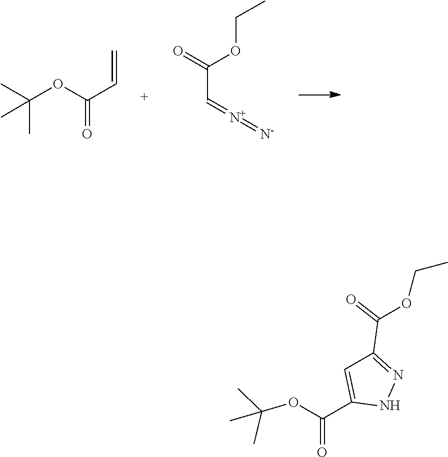

To a solution of tert-butyl acrylate (2.0 mL, 13.7 mmol) in MeCN (30 mL) were added ethyl diazoacetate (2.6 mL, 21.8 mmol), tert-butyl hydroperoxide solution (Luperox® TBH70X)(70 wt % in water)(3.8 mL, 27.3 mmol) and tetrabutylammonium iodide (504 mg, 1.37 mmol). The mixture was stirred at 40° C. for 20 h.

The solution was quenched with sat. Na₂S₂O₃ (aq)(50 mL) and diluted with EtOAc (150 ml). The organic layer was separated, washed with sat. NaHCO₃(aq)(50 ml) and brine (50 ml), dried (Na₂SO₄) and evaporated. Flash chromatography (silica, 2-50% EtOAc in iso-hexane) afforded the product (3.10 g, 94% yield).

[M−^{t}Bu+H]⁺=185.1

¹H NMR (DMSO-d6, 400 MHZ) δ 1.29 (3H, t, J=6.8 Hz), 1.53 (9H, s), 4.23-4.39 (2H, m), 7.10 (1H, s)

### O-5-tert-Butyl O-3-ethyl 4-iodo-1H-pyrazole-3,5-dicarboxylate

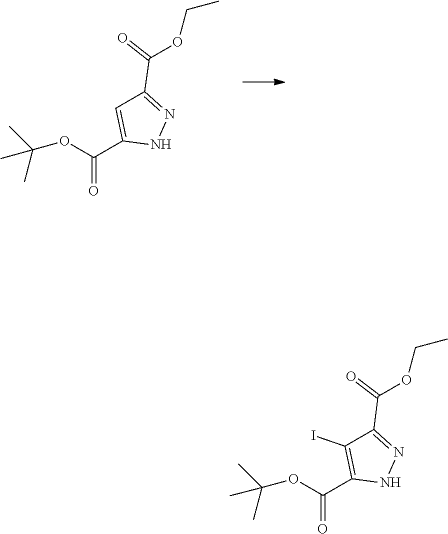 [SMILES: CCOC(=O)c1cc(C(=O)OC(C)(C)C)[nH]n1 ; CCOC(=O)c1n[nH]c(C(=O)OC(C)(C)C)c1I]

To a solution of 0-5-tert-butyl O-3-ethyl 1H-pyrazole-3,5-dicarboxylate (2.75 g, 11.4 mmol) in CHCl₃ (100 mL) was added N-iodosuccinimide (3.86 g, 17.2 mmol) and the mixture was stirred at 60° C. for 60 h. The solution was diluted with DCM (100 mL) and washed with sat. Na₂S₂O₃ (aq)(50 mL), sat. NaHCO₃(aq)(50 mL) and brine (50 mL), dried (Na₂SO₄) and evaporated. Flash chromatography (silica, 1-50% EtOAc in iso-hexane) afforded the product (3.21 g, 77% yield).

[M−^{t}Bu+H]⁺=310.9

¹H NMR (DMSO-d6, 400 MHZ) δ 1.24-1.41 (3H, m), 1.56 (9H, s), 4.21-4.38 (2H, m).

### O-5-tert-Butyl O-3-ethyl 1-(3-chlorophenyl)-4-iodo-pyrazole-3,5-dicarboxylate

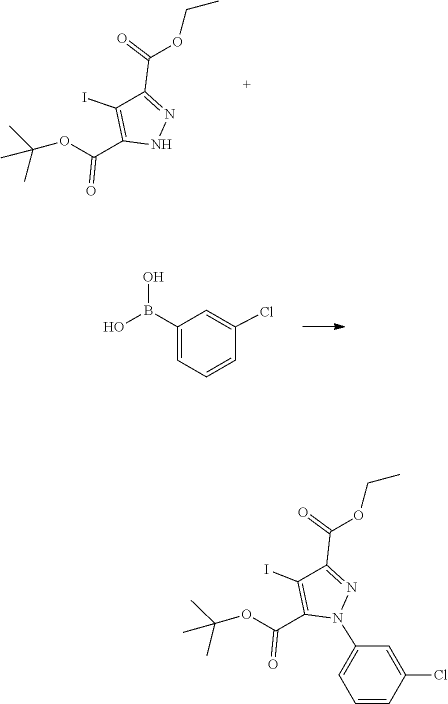 [SMILES: CCOC(=O)c1n[nH]c(C(=O)OC(C)(C)C)c1I ; OB(O)c1cccc(Cl)c1 ; CCOC(=O)c1nn(-c2cccc(Cl)c2)c(C(=O)OC(C)(C)C)c1I]

To a solution of O-5-tert-butyl O-3-ethyl 4-iodo-1H-pyrazole-3,5-dicarboxylate (3.20 g, 8.74 mmol) in DCM (60 mL) were added 3-chlorobenzeneboronic acid (2.73 g, 17.5 mmol), pyridine (4.2 mL, 52.4 mmol), copper (II) acetate (2.06 g, 11.4 mmol) and 4 Å molecular sieves (1.5 g, 50% w/w), and the mixture was stirred under air at rt for 80 h. After this time, further 3-chlorobenzeneboronic acid (500 mg, 3.15 mmol), pyridine (2 mL, 26 mmol), copper (II) acetate (500 m g, 2.85 mmol) and 4 Å molecular sieves (1 g, 33% w/w) were added, and the mixture was stirred at rt for a further 24 h. The suspension was filtered through Celite® and washed with DCM (150 mL). The combined filtrates were washed with a sat. NaHCO₃(aq)(2×50 mL), 1M HCl (aq)(50 mL) and brine (50 mL), dried (Na₂SO₄), filtered and evaporated. Flash chromatography (silica, 0-30% EtOAc in iso-hexane) afforded the product (4.12 g, 99% yield).

[M+H]⁺=477.0/479.0

¹H NMR (DMSO-d6, 400 MHZ) δ 1.07 & 1.30 (3H, t, J=7.1 Hz), 1.30 & 1.56 (9H, s), 4.19 & 4.33 (2H, q, J=7.1 Hz), 7.47-7.53 (1H, m), 7.55-7.60 (1H, m), 7.61-7.66 (1H, m), 7.68-7.76 (1H, m)

### O-5-tert-Butyl O-3-ethyl 4-[(tert-butoxycarbonylamino)methyl]-1-(3-chlorophenyl) pyrazole-3,5-dicarboxylate

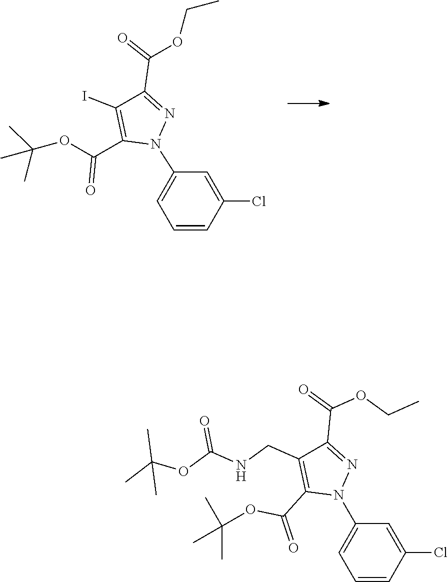 [SMILES: CCOC(=O)c1nn(-c2cccc(Cl)c2)c(C(=O)OC(C)(C)C)c1I ; CCOC(=O)c1nn(-c2cccc(Cl)c2)c(C(=O)OC(C)(C)C)c1CNC(=O)OC(C)(C)C]

A solution of O-5-tert-butyl O-3-ethyl 1-(3-chlorophenyl)-4-iodo-pyrazole-3,5-dicarboxylate (2.00 g, 4.20 mmol) and potassium (((tert-butoxycarbonyl) amino) methyl) trifluoroborate (1.49 g, 6.29 mmol) in a mixture of 1,4-dioxane (32 mL) and water (8 mL) was purged with N₂ (g) for 5 min. After this time Cs₂CO₃ (2.73 g, 8.39 mmol), di (1-adamantyl)-n-butylphosphine (cataCXium A)(301 mg, 0.84 mmol) and, finally, CataCXium A Pd G3 (306 mg, 0.42 mmol) were added sequentially. The mixture was stirred at 90° C. for 20 h. The suspension was filtered through Celite® and washed with EtOAc (150 mL). The filtrate was washed with sat. NaHCO₃(aq)(50 ml) and brine (50 ml), dried (Na₂SO₄) and evaporated. Flash chromatography (silica, 0-20% EtOAc in iso-hexane) afforded the product (775 mg, 38% yield).

[M−Boc−^{t}Bu+H]⁺=324.1/326.1

¹H NMR (DMSO-d6, 400 MHZ) δ 1.30 (3H, t, J=7.1 Hz), 1.34 & 1.54 (9H, s), 1.37 (9H, s), 4.18 & 4.32 (2H, q, J=7.1 Hz), 4.49-4.53 (2H, m), 6.74 (1H, s br), 7.42-7.48 (1H, m), 7.53-7.66 (3H, m)

### 4-(Aminomethyl)-2-(3-chlorophenyl)-5-ethoxycarbonyl-pyrazole-3-carboxylic acid hydrochloride

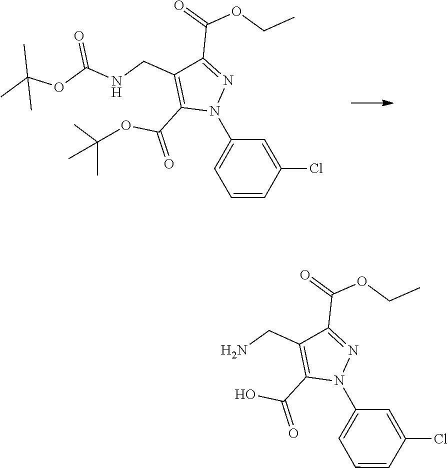 [SMILES: CCOC(=O)c1nn(-c2cccc(Cl)c2)c(C(=O)OC(C)(C)C)c1CNC(=O)OC(C)(C)C ; CCOC(=O)c1nn(-c2cccc(Cl)c2)c(C(=O)O)c1CN]

To a solution of O-5-tert-butyl O-3-ethyl 4-[(tert-butoxycarbonylamino)methyl]-1-(3-chlorophenyl) pyrazole-3,5-dicarboxylate (770 mg, 1.60 mmol) in 1,4-dioxane (4 mL) was added 4M HCl in 1,4-dioxane (12 mL, 48.0 mmol). After stirring at rt for 48 h the solvent was evaporated to afford the product (600 mg, 100% yield).

[M+H]⁺=324.2/326.

¹H NMR (DMSO-d6, 400 MHZ) δ 1.14 & 1.34 (3H, t, J=7.1 Hz), 4.22 & 4.36 (2H, q, J=7.1 Hz), 4.41-4.48 (2H, m), 7.43-7.50 (1H, m), 7.54-7.61 (1H, m), 7.60-7.63 (1H, m), 7.64-7.68 (1H, m), 8.20-8.25 (3H, m)

### Ethyl 1-(3-chlorophenyl)-6-oxo-4,5-dihydropyrrolo[3,4-c]pyrazole-3-carboxylate

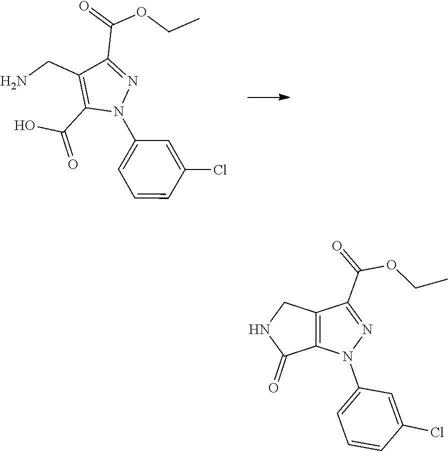 [SMILES: CCOC(=O)c1nn(-c2cccc(Cl)c2)c(C(=O)O)c1CN ; CCOC(=O)c1nn(-c2cccc(Cl)c2)c2c1CNC2=O]

To a suspension of 4-(aminomethyl)-2-(3-chlorophenyl)-5-ethoxycarbonyl-pyrazole-3-carboxylic acid hydrochloride (560 mg, 1.55 mmol) in dry MeCN (70 mL) were added 1-methylimidazole (0.62 mL, 7.77 mmol) and TCFH (567 mg, 2.02 mmol), and the mixture was stirred at rt for 3 h. The solvent was evaporated and the residue was dissolved in EtOAc (70 mL). This solution was washed with sat. NaHCO₃(aq)(50 ml) and brine (50 ml), dried (Na₂SO₄) and evaporated. Flash chromatography (silica, 5-100% EtOAc in iso-hexane) afforded ethyl 1-(3-chlorophenyl)-6-oxo-4,5-dihydropyrrolo[3,4-c]pyrazole-3-carboxylate (372 mg) and the undesired regioisomer that had been carried through, ethyl 2-(3-chlorophenyl)-6-oxo-4,5-dihydropyrrolo[3,4-c]pyrazole-3-carboxylate (176 mg). Ethyl 1-(3-chlorophenyl)-6-oxo-4,5-dihydropyrrolo[3,4-c]pyrazole-3-carboxylate

[M+H]⁺=306.2/308.2.

¹H NMR (DMSO-d6, 400 MHz) δ 1.33 (3H, t, J=7.1 Hz), 4.35 (2H, q, J=7.1 Hz), 4.41 (2H, d, J=1.2 Hz), 7.49 (1H, m), 7.61 (1H, t, J=8.1 Hz), 8.24 (1H, m), 8.45 (1H, t, J=2.0 Hz), 9.04 (1H, s)

### Ethyl 2-(3-chlorophenyl)-6-oxo-4,5-dihydropyrrolo[3,4-c]pyrazole-3-carboxylate

[M+H]⁺=306.1/308.0

¹H NMR (DMSO-d6, 400 MHZ) δ 1.20 (3H, t, J=7.1 Hz), 4.20 (2H, q, J=7.1 Hz), 4.43 (2H, d, J=1.1 Hz), 7.55 -7.59 (2H, m), 7.60-7.64 (1H, m), 7.76-7.78 (1H, m), 8.77 (1H, s)

### tert-Butyl 7-[2-(3-chlorophenyl)-3-ethoxycarbonyl-6-oxo-4H-pyrrolo[3,4-c]pyrazol-5-yl]-3,4-dihydro-1H-isoquinoline-2-carboxylate

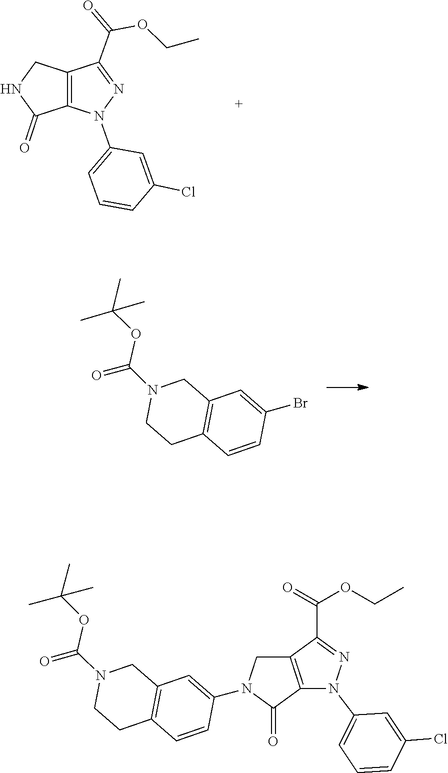 [SMILES: CCOC(=O)c1nn(-c2cccc(Cl)c2)c2c1CNC2=O ; CC(C)(C)OC(=O)N1CCc2ccc(Br)cc2C1 ; CCOC(=O)c1nn(-c2cccc(Cl)c2)c2c1CN(c1ccc3c(c1)CN(C(=O)OC(C)(C)C)CC3)C2=O]

A solution of ethyl 1-(3-chlorophenyl)-6-oxo-4,5-dihydropyrrolo[3,4-c]pyrazole-3-carboxylate (175 mg, 0.572 mmol) and tert-butyl 7-bromo-3,4-dihydro-1H-isoquinoline-2-carboxylate (250 mg, 0.801 mmol) in dry 1,4-dioxane (5.5 mL) was purged with N₂ (g) for 5 min. Tripotassium phosphate (365 mg, 1.72 mmol), (1S,2S)—N,N′-dimethyl-1,2-cyclohexanediamine (33 mg, 0.23 mmol) and, finally, Cu(I)I (22 mg, 0.11 mmol) were added. The system was closed and stirred at 100° C. for 20 h. The suspension was filtered through Celite® and washed with EtOAc (150 mL). The combined filtrates were washed with sat. NaHCO₃(aq)(50 ml) and brine (50 ml), dried (Na₂SO₄) and evaporated. Flash chromatography (silica, 10-60% EtOAc in tert-butyliso-hexane) afforded the product (185 mg, 60% yield).

[M−^{t}Bu+H]⁺=481.1/483

¹H NMR (DMSO-d6, 400 MHZ) δ 1.24 (3H, t, J=7.1 Hz), 1.44 (9H, s), 2.78 (2H, t, J=5.9 Hz), 3.57 (2H, t, J=5.8 Hz), 4.25 (2H,q, J=7.1 Hz), 4.55 (2H, s br), 5.07 (2H, s), 7.24 (1H, d, J=8.5 Hz), 7.55-7.66 (4H, m), 7.70 -7.75 (1H, m), 7.80-7.82 (1H, m)

### 5-(2-tert-Butoxycarbonyl-3,4-dihydro-1H-isoquinolin-7-yl)-2-(3-chlorophenyl)-6-oxo-4H-pyrrolo[3,4-c] pyrazole-3-carboxylic acid

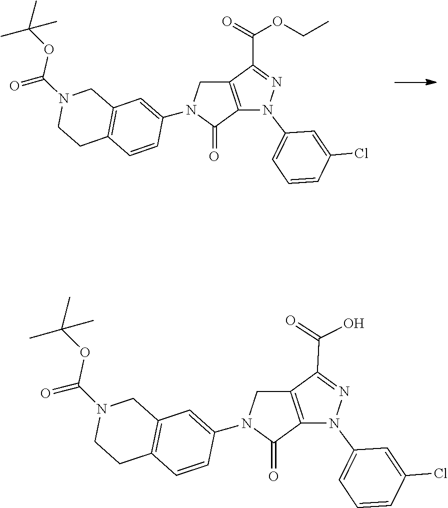 [SMILES: CCOC(=O)c1nn(-c2cccc(Cl)c2)c2c1CN(c1ccc3c(c1)CN(C(=O)OC(C)(C)C)CC3)C2=O ; CC(C)(C)OC(=O)N1CCc2ccc(N3Cc4c(C(=O)O)nn(-c5cccc(Cl)c5)c4C3=O)cc2C1]

7-[2-(3-Chlorophenyl)-3-ethoxycarbonyl-6-oxo-4H-pyrrolo[3,4-c]pyrazol-5-yl]-3,4-dihydro-1H-isoquinoline-2-carboxylate (180 mg, 0.33 mmol) was dissolved in a mixture of THF (3 mL) and water (1.5 mL). LiOH·H₂O (35 mg, 0.84 mmol) was added and the mixture was stirred at rt for 18 h. The mixture was neutralised with the addition of 1M HCl (aq)(0.9 mL) and evaporated to afford the product (228 mg, 100% yield).

[M−^{t}Bu+H]⁺=453.1/455.1

### tert-Butyl 7-[2-(3-chlorophenyl)-3-[(1,5-dimethylpyrazol-3-yl)methylcarbamoyl]-6-oxo-4H-pyrrolo[3,4-c] pyrazol-5-yl]-3,4-dihydro-1H-isoquinoline-2-carboxylate

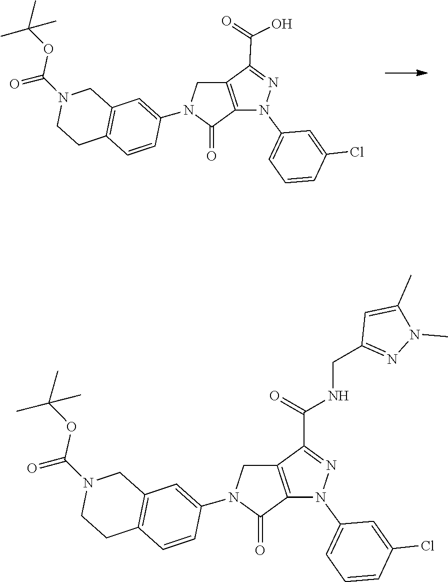 [SMILES: CC(C)(C)OC(=O)N1CCc2ccc(N3Cc4c(C(=O)O)nn(-c5cccc(Cl)c5)c4C3=O)cc2C1 ; Cc1cc(CNC(=O)c2nn(-c3cccc(Cl)c3)c3c2CN(c2ccc4c(c2)CN(C(=O)OC(C)(C)C)CC4)C3=O)nn1C]

To a suspension of 5-(2-tert-butoxycarbonyl-3,4-dihydro-1H-isoquinolin-7-yl)-2-(3-chlorophenyl)-6-oxo-4H-pyrrolo[3,4-c]pyrazole-3-carboxylic acid (170 mg, 0.33 mmol) in MeCN (5 mL) were added (1,5-dimethyl-1H-pyrazol-3-yl) methanamine (0.056 mL,0.50 mmol), 1-methylimidazole (0.19 mL, 2.34 mmol) and TCFH (141 mg, 0.50 mmol). After stirring at rt for 20 h the solution was diluted with EtOAc (50 mL), washed with sat. NaHCO₃(aq)(15 ml) and brine (15 ml), dried (Na₂SO₄) and evaporated. Flash chromatography (silica, 0-20% MeOH in DCM) afforded the product (142 mg, 69% yield).

[M−^{t}Bu+H]⁺=560.1/562.2

¹H NMR (DMSO-d6, 400 MHZ) δ 1.44 (9H, s), 2.22 (3H, d, J=0.7 Hz), 2.78 (2H, t, J=5.9 Hz), 3.57 (2H, t, J=5.9 Hz), 3.67 (3H, s), 4.30 (2H, d, J=5.9 Hz), 4.52-4.56 (2H, m), 5.09 (2H, s), 5.92 (1H, d, J=0.8 Hz), 7.24 (1H, d, J=8.4 Hz), 7.52-7.62 (5H, m), 7.69 (1H, dd, J=8.4, 2.3 Hz), 9.12 (1H, t, J=5.9 Hz)

### 2-(3-Chlorophenyl)-N-[(1,5-dimethylpyrazol-3-yl)methyl]-6-oxo-5-(1,2,3,4-tetrahydroisoquinolin-7-yl)-4H-pyrrolo[3,4-c]pyrazole-3-carboxamide hydrochloride

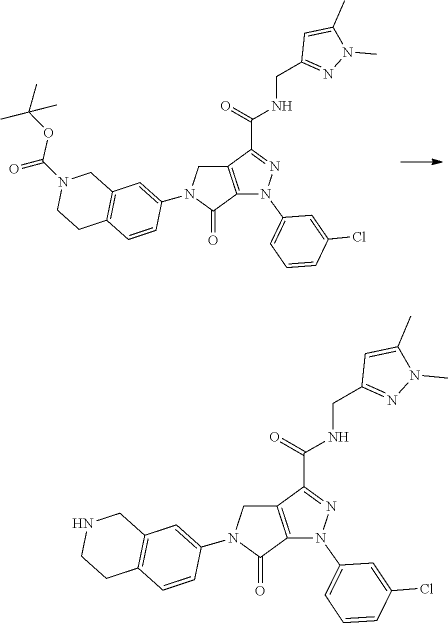 [SMILES: Cc1cc(CNC(=O)c2nn(-c3cccc(Cl)c3)c3c2CN(c2ccc4c(c2)CN(C(=O)OC(C)(C)C)CC4)C3=O)nn1C ; Cc1cc(CNC(=O)c2nn(-c3cccc(Cl)c3)c3c2CN(c2ccc4c(c2)CNCC4)C3=O)nn1C]

To a suspension of tert-butyl 7-[2-(3-chlorophenyl)-3-[(1,5-dimethylpyrazol-3-yl)methylcarbamoyl]-6-oxo-4H-pyrrolo[3,4-c]pyrazol-5-yl]-3,4-dihydro-1H-isoquinoline-2-carboxylate (140 mg, 0.23 mmol) in 1,4-dioxane (1.5 mL) was added 4M HCl in 1,4-dioxane (1.5 mL, 6.00 mmol). The mixture was stirred at rt for 20 h. The solvent was evaporated to afford the product (130 mg, 100% yield).

[M+H]⁺=516.2/518.2

### 5-[2-[2-[tert-Butyl (dimethyl) silyl]oxyethyl]-3,4-dihydro-1H-isoquinolin-7-yl]-1-(3-chlorophenyl)-N-[(1,5-dimethylpyrazol-3-yl)methyl]-6-oxo-4H-pyrrolo[3,4-c]pyrazole-3-carboxamide

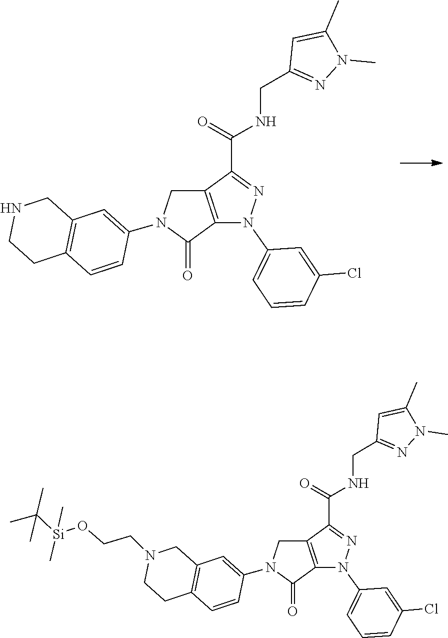

To a solution of 1-(3-chlorophenyl)-N-[(1,5-dimethylpyrazol-3-yl)methyl]-6-oxo-5-(1,2,3,4-tetrahydroisoquinolin-7-yl)-4H-pyrrolo[3,4-c]pyrazole-3-carboxamide hydrochloride (85 mg, 0.16 mmol) in a mixture of DCM (2 mL) and MeOH (0.50 mL) were added TEA (0.092 mL, 0.66 mmol) and (tert-butyldimethylsilyloxy) acetaldehyde (0.07 mL, 0.33 mmol) and the mixture was stirred at rt for 1 h. STAB (52 mg, 0.25 mmol) was added portionwise and the mixture was stirred for a further 20 h. The solution was diluted with DCM (50 mL), washed with sat. NaHCO₃(aq)(15 ml) and brine (15 ml), dried (Na₂SO₄) and evaporated. Flash chromatography (silica, 0-20% MeOH in DCM) afforded the product (61 mg, 55% yield).

[M+H]⁺=674.4/676.6

### 1-(3-Chlorophenyl)-N-[(1,5-dimethylpyrazol-3-yl)methyl]-5-[2-(2-hydroxyethyl)-3,4-dihydro-1H-isoquinolin-7-yl]-6-oxo-4H-pyrrolo[3,4-c]pyrazole-3-carboxamide

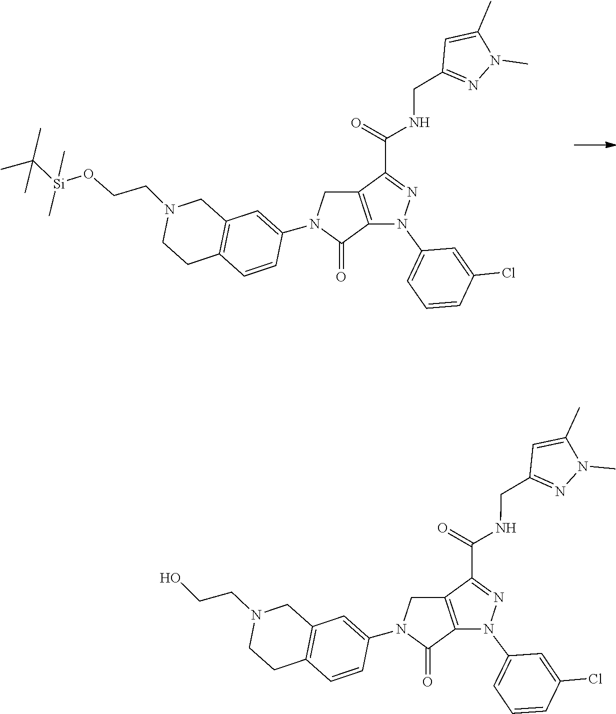 [SMILES: Cc1cc(CNC(=O)c2nn(-c3cccc(Cl)c3)c3c2CN(c2ccc4c(c2)CN(CCO[Si](C)(C)C(C)(C)C)CC4)C3=O)nn1C ; Cc1cc(CNC(=O)c2nn(-c3cccc(Cl)c3)c3c2CN(c2ccc4c(c2)CN(CCO)CC4)C3=O)nn1C]

To a suspension of 5-[2-[2-[tert-butyl(dimethyl) silyl]oxyethyl]-3,4-dihydro-1H-isoquinolin-7-yl]-1-(3-chlorophenyl)-N-[(1,5-dimethylpyrazol-3-yl)methyl]-6-oxo-4H-pyrrolo[3,4-c]pyrazole-3-carboxamide (60 mg, 0.0890 mmol) in 1,4-dioxane (1 mL) was added 4M HCl in 1,4-dioxane (0.56 mL, 2.22 mmol). After stirring at rt for 20 h the solvent was evaporated and the residue purified by loading it onto an ion-exchange SCX-2 column, washing with MeOH (10 mL) and eluting with 3M NH₃ in MeOH (10 mL). This was further purified by preparative HPLC purification (C18, 47-48.4% [MeCN+0.1% NH₃] in [10 mM NH₄CO₃ in H₂O+0.1% NH₃) and lyophilised to afford the product (11 mg, 21% yield).

[M+H]⁺=560.3/562.3

¹H NMR (DMSO-d6, 400 MHZ) δ 2.20 (3H, s), 2.57 (2H, t, J=6.2 Hz), 2.67-2.75 (2H, m), 2.77-2.83 (2H, m), 3.58-3.64 (4H, m), 3.66 (3H, s), 4.36 (2H, d, J=6.1 Hz), 4.46-4.54 (1H, m), 5.01 (2H, s), 5.96 (1H, s), 7.16 (1H, d, J=8.4 Hz), 7.46-7.55 (2H, m), 7.56-7.66 (2H, m), 8.40-8.43 (1H, m), 8.53 (1H, t, J=2.1 Hz), 9.06 (1H, t, J=6.1 Hz)

### Example Number 2020

### 1-(6-Chloropyridin-2-yl)-N-(cyclopropylmethyl)-6-(2-methyl-1-oxo-1,2,3,4-tetrahydroisoquinolin-7-yl)-7-oxo-4,5,6,7-tetrahydro-1H-pyrazolo[3,4-c]pyridine-3-carboxamide

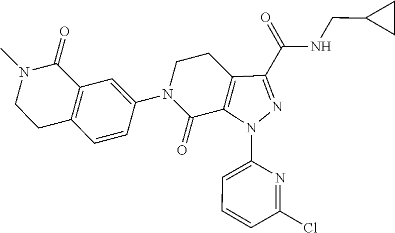 [SMILES: CN1CCc2ccc(N3CCc4c(C(=O)NCC5CC5)nn(-c5cccc(Cl)n5)c4C3=O)cc2C1=O]

### Ethyl 1-(6-chloropyridin-2-yl)-6-(2-methyl-1-oxo-1,2,3,4-tetrahydroisoquinolin-7-yl)-7-oxo-4,5,6,7-tetrahydro-1H-pyrazolo[3,4-c]pyridine-3-carboxylate

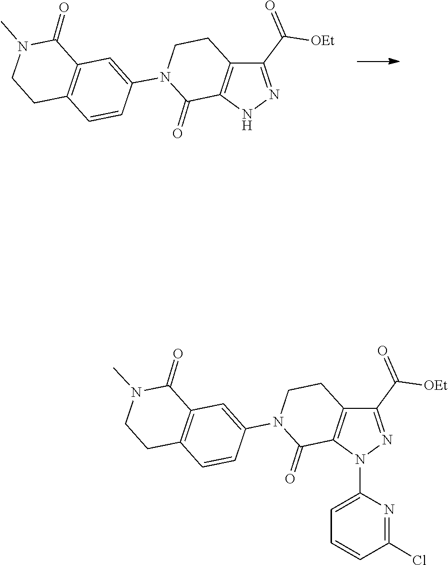 [SMILES: CCOC(=O)c1n[nH]c2c1CCN(c1ccc3c(c1)C(=O)N(C)CC3)C2=O ; CCOC(=O)c1nn(-c2cccc(Cl)n2)c2c1CCN(c1ccc3c(c1)C(=O)N(C)CC3)C2=O]

Ethyl 6-(2-methyl-1-oxo-3,4-dihydroisoquinolin-7-yl)-7-oxo-4,5-dihydro-1H-pyrazolo[3,4-c]pyridine-3-carboxylate (100 mg, 0.27 mmol), Cu(I)I (5 mg, 0.03 mmol), K₂CO₃ (79 mg, 0.57 mmol), (1S,2S)—N,N′-dimethyl-1,2-cyclohexane-diamine (15 mg, 0.11 mmol) and 3-bromo-5-chloropyridine (500 mg, 2.6 mmol) were added to a screw capped pressure vial. The reaction vessel was sealed and flushed with N₂ (g) and the mixture was stirred at 110° C. for 3 days. The reaction mixture was cooled to rt, diluted with EtOAc (50 mL) and filtered through Celite®, washing with EtOAc (3×10 mL). The filtrate was concentrated and purified via flash chromatography (Silica, 0-100% EtOAc in Pet. Ether, followed by 0-20% MeOH in EtOAc) to afford the product (77 mg, 59% yield).

[M+H]⁺=480.1

### 1-(6-Chloropyridin-2-yl)-6-(2-methyl-1-oxo-1,2,3,4-tetrahydroisoquinolin-7-yl)-7-oxo-4,5,6,7-tetrahydro-1H-pyrazolo[3,4-c]pyridine-3-carboxylic acid

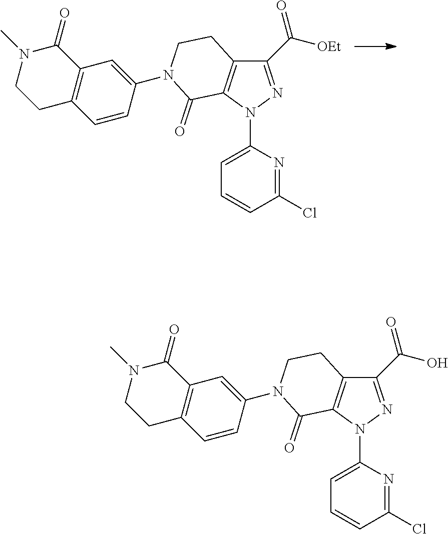 [SMILES: CCOC(=O)c1nn(-c2cccc(Cl)n2)c2c1CCN(c1ccc3c(c1)C(=O)N(C)CC3)C2=O ; CN1CCc2ccc(N3CCc4c(C(=O)O)nn(-c5cccc(Cl)n5)c4C3=O)cc2C1=O]

Ethyl 1-(6-chloropyridin-2-yl)-6-(2-methyl-1-oxo-1,2,3,4-tetrahydroisoquinolin-7-yl)-7-oxo-4,5,6,7-tetrahydro-1H-pyrazolo[3,4-c]pyridine-3-carboxylate (77 mg, 0.12 mmol) was taken up in THF (10 mL) and water (1 mL) and treated with LiOH·H₂O (21 mg, 0.36 mmol). The mixture was stirred at rt overnight and concentrated, and the residue was taken up in water (15 mL). The solution was adjusted to pH 4˜5 using 2M HCl (aq.) and the product was extracted with 4:1 CHCl₃/IPA (3×20 mL). The combined organic layers were washed with brine, dried (MgSO₄), and concentrated to afford the product (73 mg, 100% yield).

[M+H]⁺=452.1

### 1-(6-Chloropyridin-2-yl)-N-(cyclopropylmethyl)-6-(2-methyl-1-oxo-1,2,3,4-tetrahydroisoquinolin-7-yl)-7-oxo-4,5,6,7-tetrahydro-1H-pyrazolo[3,4-c]pyridine-3-carboxamide

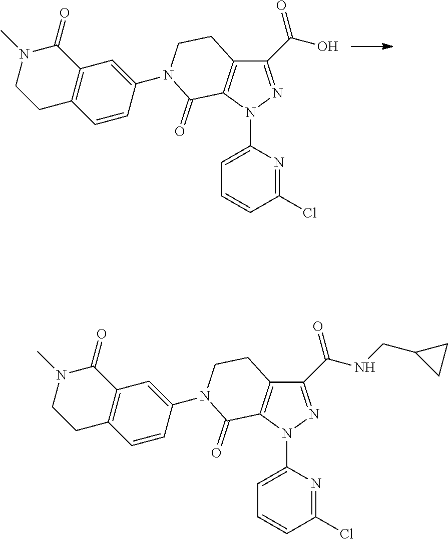 [SMILES: CN1CCc2ccc(N3CCc4c(C(=O)O)nn(-c5cccc(Cl)n5)c4C3=O)cc2C1=O ; CN1CCc2ccc(N3CCc4c(C(=O)NCC5CC5)nn(-c5cccc(Cl)n5)c4C3=O)cc2C1=O]

HBTU (74 mg, 0.19 mmol) and 1-(6-chloropyridin-2-yl)-6-(2-methyl-1-oxo-1,2,3,4-tetrahydroisoquinolin-7-yl)-7-oxo-4,5,6,7-tetrahydro-1H-pyrazolo[3,4-c]pyridine-3-carboxylic acid (73 mg, 0.16 mmol) were dissolved in a mixture of DCM (5 mL) and TEA (68 μL, 0.48 mmol). After 20 min cyclopropylmethylamine (15 μL, 0.18 mmol) was added. The mixture was stirred at rt overnight, diluted with DCM (50 mL) and washed with sat. NaHCO₃(aq)(30 mL) and water (30 mL). The combined aqueous layers were back-extracted with DCM (2×50 mL). The organic layers were combined, washed with brine (1×30 mL), dried (MgSO₄) and concentrated. The residue was purified via prep HPLC (mass directed 2-60% MeCN/Water in basic mobile phase) and further purified via prep HPLC (mass directed 2-60% MeCN/Water in acidic mobile phase) to afford the product (13 mg, 16% yield).

[M+H]⁺=505.1

¹H NMR (DMSO-d6, 400 MHZ) 5 0.19-0.28 (2H, m), 0.35-0.46 (2H, m), 0.98-1.11 (1H, m), 2.97 (2H, t, J=6.7 Hz), 3.02 (3H, s), 3.11 (2H, t, J=6.4 Hz), 3.23 (2H, t, J=6.6 Hz), 3.54 (2H, t, J=6.7 Hz), 4.10 (2H, t, J=6.6 Hz), 7.32 (1H, d, J=8.1 Hz), 7.45 (1H, dd, J=8.1, 2.3 Hz), 7.70 (1H, dd, J=7.9, 0.7 Hz), 7.76 (1H, dd, J=7.9, 0.7 Hz), 7.82 (1H, d, J=2.3 Hz), 8.10 (1H, t, J=7.9 Hz), 8.58 (1H, t, J=6.0 Hz)

### Example 2017

### 1-(3-(Aminomethyl)-5-chlorophenyl)-N-((1,5-dimethyl-1H-pyrazol-3-yl)methyl)-6-(2-methyl-1-oxo-1,2,3,4-tetrahydroisoquinolin-7-yl)-7-oxo-4,5,6,7-tetrahydro-1H-pyrazolo[3,4-c]pyridine-3-carboxamide

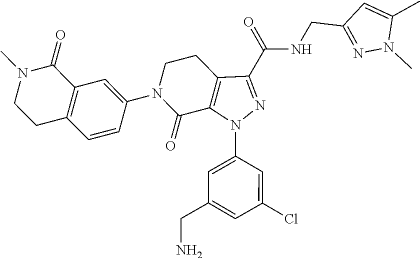

### Ethyl 1-(3-chloro-5-cyanophenyl)-6-(2-methyl-1-oxo-1,2,3,4-tetrahydroisoquinolin-7-yl)-7-oxo-4,5,6,7-tetrahydro-1H-pyrazolo[3,4-c]pyridine-3-carboxylate

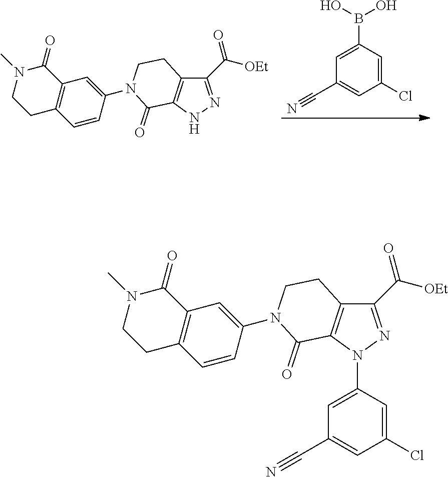 [SMILES: CCOC(=O)c1n[nH]c2c1CCN(c1ccc3c(c1)C(=O)N(C)CC3)C2=O ; N#Cc1cc(Cl)cc(B(O)O)c1 ; CCOC(=O)c1nn(-c2cc(Cl)cc(C#N)c2)c2c1CCN(c1ccc3c(c1)C(=O)N(C)CC3)C2=O]

Ethyl 6-(2-methyl-1-oxo-1,2,3,4-tetrahydroisoquinolin-7-yl)-7-oxo-4,5,6,7-tetrahydro-1H-pyrazolo[3,4-c]pyridine-3-carboxylate (300 mg, 0.81 mmol) and (3-chloro-5-cyanophenyl) boronic acid (162 mg, 0.9 mmol) were dissolved in DCM (10 mL). Pyridine (66 μL, 0.81 mmol) and Cu (II)(OAc) 2.H₂O (179 mg, 0.9 mmol) were added and the mixture was stirred at 40° C. for 4 days. The mixture was cooled to rt, diluted with EtOAc (50 mL) and filtered through Celite®, washing with EtOAc (3×10 mL). The filtrate was concentrated, and the residue was purified via flash chromatography (Silica, 0-100% EtOAc in Pet. Ether followed by 0-20% MeOH in EtOAc) to afford the product (159 mg, 39% yield).

[M+H]⁺=504.1

### 1-(3-Chloro-5-cyanophenyl)-6-(2-methyl-1-oxo-1,2,3,4-tetrahydroisoquinolin-7-yl)-7-oxo-4,5,6,7-tetrahydro-1H-pyrazolo[3,4-c]pyridine-3-carboxylic acid

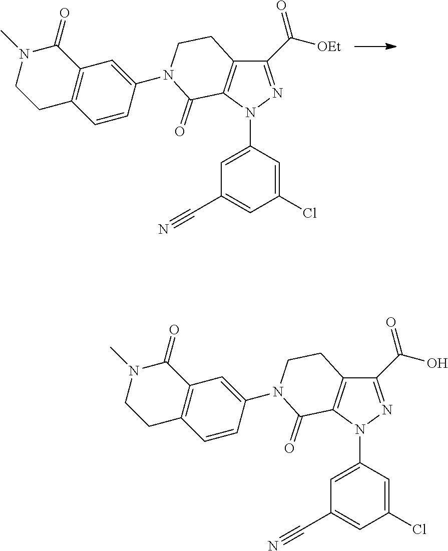 [SMILES: CCOC(=O)c1nn(-c2cc(Cl)cc(C#N)c2)c2c1CCN(c1ccc3c(c1)C(=O)N(C)CC3)C2=O ; CN1CCc2ccc(N3CCc4c(C(=O)O)nn(-c5cc(Cl)cc(C#N)c5)c4C3=O)cc2C1=O]

Ethyl 1-(3-chloro-5-cyanophenyl)-6-(2-methyl-1-oxo-1,2,3,4-tetrahydroisoquinolin-7-yl)-7-oxo-4,5,6,7-tetrahydro-1H-pyrazolo[3,4-c]pyridine-3-carboxylate (159 mg, 0.32 mmol) was taken up in THF (10 mL) and water (1 mL) and treated with LiOH·H₂O (54 mg, 0.95 mmol). The mixture was stirred at rt overnight, concentrated and taken up in water (15 mL). The mixture was adjusted to pH 4-5 using 2M HCl (aq) and the product was extracted with 4:1 CHCl₃/IPA (3×20 mL). The combined organic layers were washed with brine, dried (MgSO₄) and concentrated to afford the product (147 mg, 98% yield).

[M+H]⁺=476.0

### 1-(3-Chloro-5-cyanophenyl)-N-((1,5-dimethyl-1H-pyrazol-3-yl)methyl)-6-(2-methyl-1-oxo-1,2,3,4-tetrahydroisoquinolin-7-yl)-7-oxo-4,5,6,7-tetrahydro-1H-pyrazolo[3,4-c]pyridine-3-carboxamide

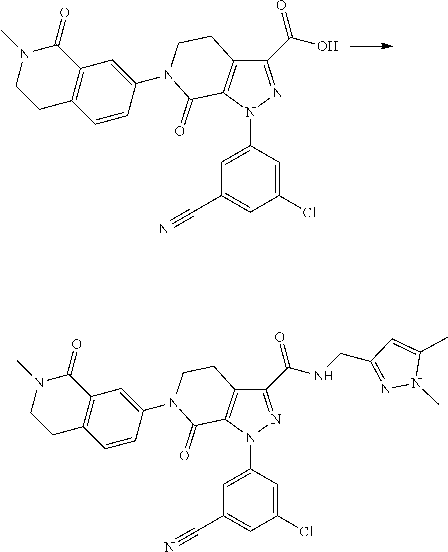

1-(3-Chloro-5-cyanophenyl)-6-(2-methyl-1-oxo-1,2,3,4-tetrahydroisoquinolin-7-yl)-7-oxo-4,5,6,7-tetrahydro-1H-pyrazolo[3,4-c]pyridine-3-carboxylic acid (74 mg, 0.16 mmol) and HBTU (71 mg, 0.19 mmol) were dissolved in a mixture of DCM (5 mL) and TEA (65 μL, 0.47 mmol). After 20 min (1,5-dimethyl-1H-pyrazol-3-yl)methylamine (21 mg, 0.17 mmol) was added, the mixture was stirred at rt overnight, diluted with DCM (50 mL) and washed with sat. NaHCO₃ (30 mL) and water (30 mL). The combined aqueous layers were back-extracted with DCM (2×50 mL). The organic layers were combined, washed with brine (30 mL), dried (MgSO₄), concentrated and purified via flash chromatography (Silica, 0-100% EtOAc in Pet. Ether followed by 0-20% MeOH in EtOAc) to afford the product (49 mg, 54% yield).

[M+H]⁺=583.2

### tert-Butyl (3-chloro-5-(3-(((1,5-dimethyl-1H-pyrazol-3-yl)methyl) carbamoyl)-6-(2-methyl-1-oxo-1,2,3,4-tetrahydroisoquinolin-7-yl)-7-oxo-4,5,6,7-tetrahydro-1H-pyrazolo[3,4-c]pyridin-1-yl)benzyl)carbamate

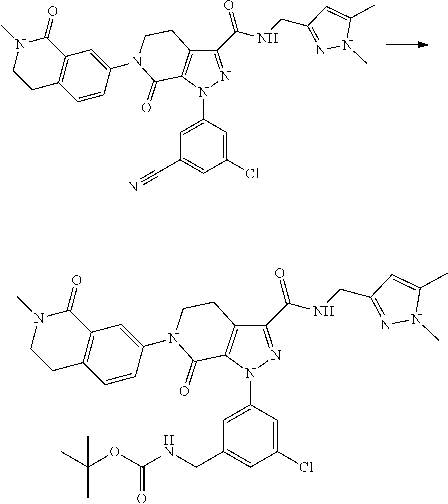

1-(3-Chloro-5-cyanophenyl)-N-((1,5-dimethyl-1H-pyrazol-3-yl)methyl)-6-(2-methyl-1-oxo-1,2,3,4-tetrahydroisoquinolin-7-yl)-7-oxo-4,5,6,7-tetrahydro-1H-pyrazolo[3,4-c]pyridine-3-carboxamide (49 mg, 0.08 mmol) was dissolved in MeOH (10 mL) and cooled in an ice/water bath. Boc₂O (37 mg, 0.17 mmol) and NiCl₂·6H₂O (2 mg) were added and the mixture was stirred for 10 min. NaBH₄ (16 mg, 0.42 mmol) was added portion-wise while continuing to cool in an ice/water bath. The mixture was warmed to rt and stirred overnight, after which time the mixture was cooled in an ice/water bath and retreated with NiCl₂·6H₂O (2 mg) and NaBH₄ (6 mg, 0.17 mmol). The mixture was stirred at rt for 4 h before the addition of diethylenetriamine (9 μL, 0.08 mmol). The mixture was stirred for 30 min, concentrated and diluted with EtOAc (20 mL), water (10 mL) and sat. NaHCO₃(aq)(10 mL). The layers were separated and the aqueous was back-extracted with EtOAc (2×20 mL). The combined organic layers were washed with brine (10 mL), dried (MgSO₄) and concentrated to afford the product (36 mg, 31% yield).

[M+H]⁺=688.1

### 1-(3-(Aminomethyl)-5-chlorophenyl)-N-((1,5-dimethyl-1H-pyrazol-3-yl)methyl)-6-(2-methyl-1-oxo-1,2,3,4-tetrahydroisoquinolin-7-yl)-7-oxo-4,5,6,7-tetrahydro-1H-pyrazolo[3,4-c]pyridine-3-carboxamide

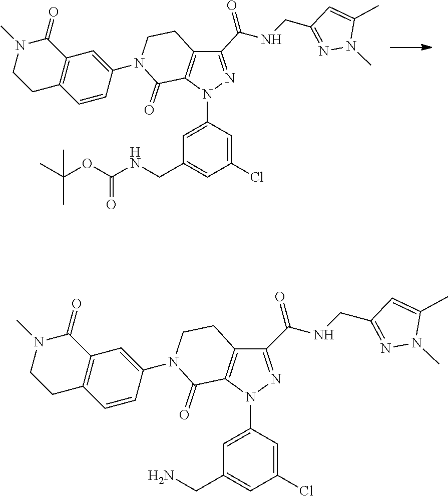

tert-Butyl (3-chloro-5-(3-(((1,5-dimethyl-1H-pyrazol-3-yl)methyl) carbamoyl)-6-(2-methyl-1-oxo-1,2,3,4-tetrahydroisoquinolin-7-yl)-7-oxo-4,5,6,7-tetrahydro-1H-pyrazolo[3,4-c]pyridin-1-yl)benzyl)carbamate (36 mg, 0.03 mmol) was taken up in DCM (2 mL). TFA (1 mL, 13.1 mmol) was added and mixture was stirred at rt for 30 min, concentrated, taken up in MeOH (2 mL) and loaded onto an SCX resin. The resin was washed with MeOH (30 mL) and the product was eluted with 2M NH₃ in MeOH (30 mL). The solution was concentrated, purified via prep HPLC (mass directed 2-60% MeCN/H2O in basic mobile phase) and followed by further purification via prep HPLC (mass directed 2-60% MeCN/H2O in acidic mobile phase) to afford the formate salt of the product (14 mg, 83% yield).

¹H NMR (DMSO-d6, 400 MHZ) δ 2.19 (3H, s), 2.97 (2H, t, J=6.7 Hz), 3.02 (3H, s), 3.21 (2H, t, J=6.6 Hz), 3.55 (2H, t, J=6.7 Hz), 3.64 (3H, s), 3.80 (2H, s), 4.07 (2H, t, J=6.6 Hz), 4.31 (2H, d, J=6.1 Hz), 5.94 (1H, s), 7.32 (1H, d, J=8.2 Hz), 7.45 (1H, dd, J=8.1, 2.4 Hz), 7.52 (1H, t, J=1.8 Hz), 7.59 (1H, t, J=1.7 Hz), 7.64 (1H, t, J=2.0 Hz), 7.82 (1H, d, J=2.3 Hz), 8.31 (1H, s), 8.64 (1H, t, J=6.1 Hz) 2 NH and 1 formic acid OH protons not observed

[M+H]⁺=587.2

### Example number 1353

### 7-(1-(3-Chlorophenyl)-3-(4,5-dihydrooxazol-2-yl)-7-oxo-1,4,5,7-tetrahydro-6H-pyrazolo[3,4-c]pyridin-6-yl)-2-methyl-3,4-dihydroisoquinolin-1 (2H)-one

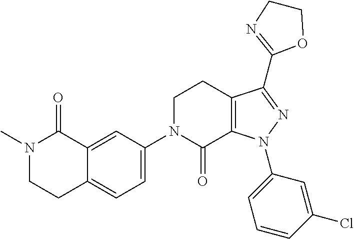 [SMILES: CN1CCc2ccc(N3CCc4c(C5=NCCO5)nn(-c5cccc(Cl)c5)c4C3=O)cc2C1=O]

### 1-(3-Chlorophenyl)-N-(2-hydroxyethyl)-6-(2-methyl-1-oxo-3,4-dihydroisoquinolin-7-yl)-7-oxo-3a,4,5,7a-tetrahydropyrazolo[3,4-c]pyridine-3-carboxamide

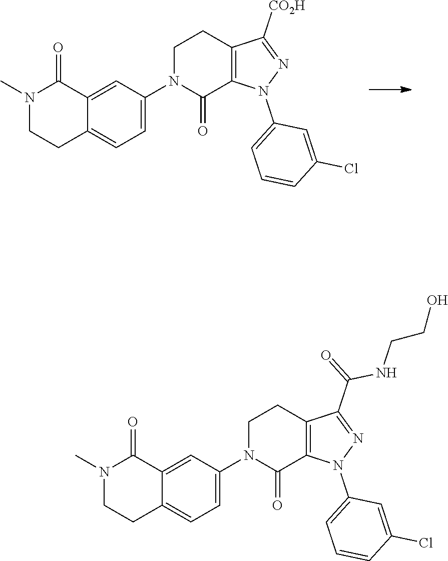 [SMILES: CN1CCc2ccc(N3CCc4c(C(=O)O)nn(-c5cccc(Cl)c5)c4C3=O)cc2C1=O ; CN1CCc2ccc(N3CCc4c(C(=O)NCCO)nn(-c5cccc(Cl)c5)c4C3=O)cc2C1=O]

Isobutyl chloroformate (20 μL, 0.152 mmol) was added to a mixture of 1-(3-chlorophenyl)-6-(2-methyl-1-oxo-3,4-dihydroisoquinolin-7-yl)-7-oxo-3a,4,5,7a-tetrahydropyrazolo[3,4-c]pyridine-3-carboxylic acid (60 mg, 0.132 mmol) and 4-methylmorpholine (22 μL, 0.200 mmol) in DCM (1.5 mL) at 0° C. 2-Aminoethanol (9 μL, 0.15 mmol) was added and the mixture brought to rt and stirred for 2 h and diluted with water (2 mL). The mixture was concentrated and purified by flash column chromatography (silica, 0-30% MeOH in EtOAc) to provide the product (60 mg, 91%).

[M+H]⁺=497.0

### 7-[1-(3-chlorophenyl)-3-(4,5-dihydrooxazol-2-yl)-7-oxo-3a,4,5,7atetrahydropyrazolo[3,4-c]pyridin-6-yl]-2-methyl-3,4-dihydroisoquinolin-1-one

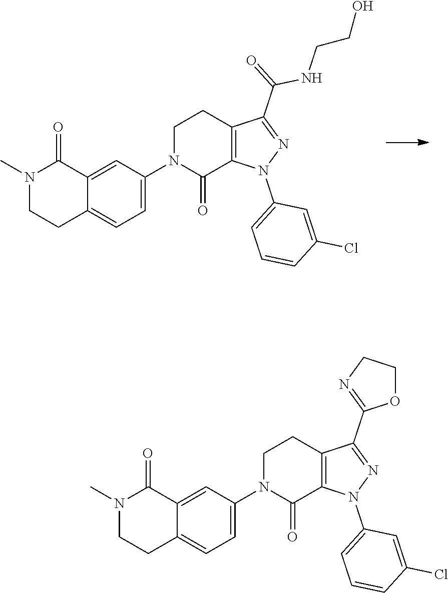 [SMILES: CN1CCc2ccc(N3CCc4c(C(=O)NCCO)nn(-c5cccc(Cl)c5)c4C3=O)cc2C1=O ; CN1CCc2ccc(N3CCc4c(C5=NCCO5)nn(-c5cccc(Cl)c5)c4C3=O)cc2C1=O]

TEA (72 μL, 0.52 mmol) was added to a mixture of 4-methylbenzenesulfonyl chloride (30 mg, 0.157 mmol) and DMAP (12 mg, 0.098 mmol) in DCE (1.5 mL) at 0° C. and stirred for 10 min. 1-(3-Chlorophenyl)-N-(2-hydroxyethyl)-6-(2-methyl-1-oxo-3,4-dihydroisoquinolin-7-yl)-7-oxo-3a,4,5,7a-tetrahydropyrazolo[3,4-c]pyridine-3-carboxamide (60 mg, 0.12 mmol) in DCE (1.5 mL) was added and the mixture brought to rt and stirred for 24 h. The mixture was stirred at 60° C. for 2 h and diluted with water (2 mL). The aqueous phase was extracted with 9:1 DCM/MeOH (3×15 mL), dried (MgSO₄), filtered, and concentrated. Purification by preparative HPLC (UV directed, 34-44% MeCN in 10 mM (NH₄) HCO₃ (aq) over 12 min) and lyophilisation provided the product (11.5 mg, 20%).

[M+H]⁺=476.5

¹H NMR (500 MHZ, DMSO-d6) δ 2.98 (t, J=6.7 Hz, 2H), 3.03 (s, 3H), 3.20 (t, J=6.6 Hz, 2H), 3.55 (t, J=6.7 Hz, 2H), 4.00 (t, J=9.6 Hz, 2H), 4.08 (t, J=6.6 Hz, 2H), 4.40 (t, J=9.5 Hz, 2H), 7.32 (d, J=8.1 Hz, 1H), 7.46 (dd, J=8.0, 2.4 Hz, 1H), 7.48-7.53 (m, 2H), 7.57-7.61 (m, 1H), 7.72-7.76 (m, 1H), 7.83 (d, J=2.4 Hz, 1H)

### Example number 1388

### 1-(3-chlorophenyl)-6-(2-methyl-1-oxo-1,2,3,4-tetrahydroisoquinolin-7-yl)-7-oxo-4,5,6,7-tetrahydro-1H-pyrazolo[3,4-c]pyridine-3-carbonitrile

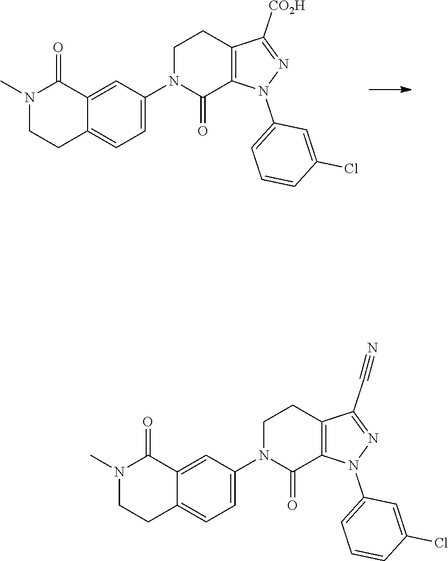 [SMILES: CN1CCc2ccc(N3CCc4c(C(=O)O)nn(-c5cccc(Cl)c5)c4C3=O)cc2C1=O ; CN1CCc2ccc(N3CCc4c(C#N)nn(-c5cccc(Cl)c5)c4C3=O)cc2C1=O]

T3P (267 μL, 0.0998 mmol) was added to 1-(3-chlorophenyl)-6-(2-methyl-1-oxo-3,4-dihydroisoquinolin-7-yl)-7-oxo-4,5-dihydropyrazolo[3,4-c]pyridine-3-carboxylic acid (40 mg, 0.089 mmol), 2-methylpropan-2-amine (28 μL,0.27 mmol) and TEA (37 μL, 0.27 mmol) in EtOAc (3 mL) at rt. The mixture was stirred at 65° C. for 30 min and concentrated. The residue was dissolved in POCl₃ (700 μL, 0.749 mmol), stirred at 70° C. for 1 h and concentrated. This was purified by reverse phase chromatography (C18, 10-15% MeCN in 10 mM (NH₄) HCO₃ (aq)) followed by automated prep HPLC (UV directed, 67-77% MeCN in 10 mM (NH₄) HCO₃ (aq) over 11 min) to afford the product (10.6 mg, 28%).

[M+H]⁺=432.5

¹H NMR (400 MHZ, DMSO-d6) δ 2.98 (2H, t, J=6.6 Hz), 3.03 (3H, s), 3.16 (2H, t, J=6.5 Hz), 3.55 (2H, t, J=6.6 Hz), 4.10 (2H, t, J=6.5 Hz), 7.33 (1H, d, J=8.1 Hz), 7.41-7.48 (1H, m), 7.51-7.61 (2H, m), 7.61-7.67 (1H, m), 7.77-7.86 (2H, m)

### Example Number 1354

### 7-(1-(3-chlorophenyl)-3-(((3-methyloxetan-3-yl)oxy) methyl)-7-oxo-1,4,5,7-tetrahydro-6H-pyrazolo[3,4-c]pyridin-6-yl)-2-methyl-3,4-dihydroisoquinolin-1 (2H)-one

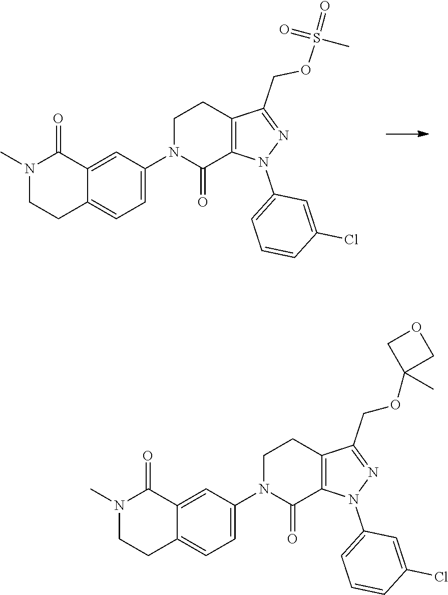 [SMILES: CN1CCc2ccc(N3CCc4c(COS(C)(=O)=O)nn(-c5cccc(Cl)c5)c4C3=O)cc2C1=O ; CN1CCc2ccc(N3CCc4c(COC5(C)COC5)nn(-c5cccc(Cl)c5)c4C3=O)cc2C1=O]

NaH (60% dispersion in oil, 23 mg, 0.58 mmol) was added to 3-methyloxetan-3-ol (52 mg, 0.58 mmol) in anhydrous DMF (2 mL) at rt and was stirred at 80° C. for 1 h. At the same time, a mixture of TBAI (43 mg, 0.12 mmol) and (1-(3-chlorophenyl)-6-(2-methyl-1-oxo-1,2,3,4-tetrahydroisoquinolin-7-yl)-7-oxo-4,5,6,7-tetrahydro-1H-pyrazolo[3,4-c]pyridin-3-yl)methyl methanesulfonate (30 mg, 0.058 mmol) in DMF (2 mL) was stirred at rt for 1 h. The solutions were combined and stirred at 80° C. for 10 min and then cooled to 0° C. Water (3 mL) was added and the mixture was concentrated. Purification by flash chromatography (Silica, 0-30% MeOH in EtOAc) followed by automated prep HPLC (UV directed, 39-49% MeCN in 10 mM (NH₄) HCO₃ (aq)) and lyophilisation afforded the product (3.62 mg, 12% yield).

[M+H]⁺=507.5

¹H NMR (DMSO-d6, 500 MHz) δ 1.57 (3H, s), 2.98 (2H, t, J=6.7 Hz), 3.02-3.06 (5H, m), 3.55 (2H, t, J=6.7 Hz), 4.07 (2H, t, J=6.6 Hz), 4.33 (2H, d, J=6.8 Hz), 4.56 (2H, s), 4.59 (2H, d, J=6.5 Hz), 7.32 (1H, d, J=8.2 Hz), 7.43-7.50 (3H, m), 7.53-7.57 (1H, m), 7.65-7.69 (1H, m), 7.83 (1H, d, J=2.3 Hz)

### Example number 1367

### 1-(3-Chlorophenyl)-N-ethyl-7-oxo-6-(3-(3-oxo-2-azabicyclo[3.1.0]hexan-2-yl)phenyl)-4,5,6,7-tetrahydro-1H-pyrazolo[3,4-c]pyridine-3-carboxamide

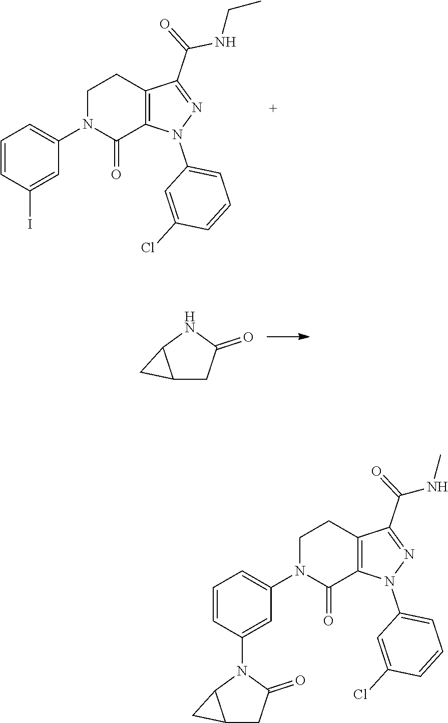 [SMILES: CCNC(=O)c1nn(-c2cccc(Cl)c2)c2c1CCN(c1cccc(I)c1)C2=O ; O=C1CC2CC2N1 ; CNC(=O)c1nn(-c2cccc(Cl)c2)c2c1CCN(c1cccc(N3C(=O)CC4CC43)c1)C2=O]

A pressure vial with Cu(I)I (11 mg, 0.06 mmol), K₃PO₄ (248 mg, 1.15 mmol), 1-(3-chlorophenyl)-N-ethyl-6-(3-iodophenyl)-7-oxo-4,5-dihydropyrazolo[3,4-c]pyridine-3-carboxamide (150 mg, 0.29 mmol), 2-azabicyclo[3.1.0]hexan-3-one (70 mg, 0.72 mmol) and 1,4-dioxane (5 mL) was purged with N₂ (g) for 5 min. N,N′-Dimethylethylenediamine (16 μL, 0.12 mmol) was added, the reaction purged with N₂ (g) for 5 min at rt, then stirred at 110° C. for 4 h. The mixture was cooled and filtered through Celite®, washed with EtOAc (3×15 mL) followed by DCM (3×15 mL). The filtrate was concentrated, and the residue was purified by automated prep HPLC (mass directed 5-95% MeCN in water with an acidic mobile phase of 2.5% formic acid in water) to afford the product (100 mg, 71% yield).

[M+H]⁺=490.1

¹H NMR (DMSO-d6, 400 MHZ) δ 0.47 (1H, td, J=5.3, 2.3 Hz), 1.11 (3H, t, J=7.2 Hz), 1.55-1.62 (1H, m), 2.97 (1H, dd, J=18.2, 7.3 Hz), 3.25 (4H, dt, J=26.8, 6.7 Hz), 3.55 (1H, t, J=6.0 Hz), 4.08 (2H, t, J=6.6 Hz), 7.15 (1H, ddd, J=8.0, 2.1, 1.0 Hz), 7.41 (1H, t, J=8.1 Hz), 7.46-7.58 (3H, m), 7.63 (1H, dt, J=6.8, 2.1 Hz), 7.69 (1H, t, J=2.0 Hz), 7.79 (1H, dt, J=2.4, 1.2 Hz), 8.46 (1H, t, J=5.9 Hz) 2H obscured by DMSO peak

### Examples numbers 1407 and 1408

### 1-(3-Chlorophenyl)-N-ethyl-7-oxo-6-(3-((1R*,5R*)-3-oxo-2-azabicyclo[3.1.0]hexan-2-yl)phenyl)-4,5,6,7-tetrahydro-1H-pyrazolo[3,4-c]pyridine-3-carboxamide (Example 1407) and

### 1-(3-Chlorophenyl)-N-ethyl-7-oxo-6-(3-((1S*,5S*)-3-oxo-2-azabicyclo[3.1.0]hexan-2-yl)phenyl)-4,5,6,7-tetrahydro-1H-pyrazolo[3,4-c]pyridine-3-carboxamide (Example 1408)

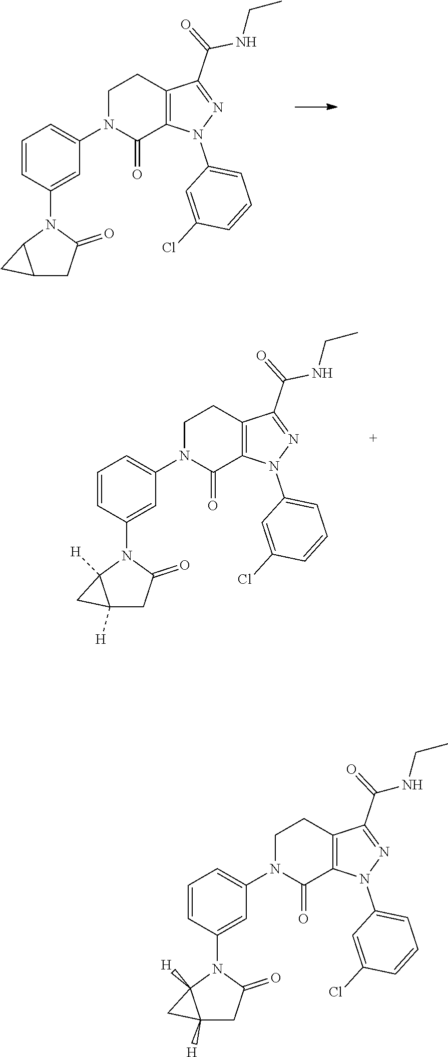 [SMILES: CCNC(=O)c1nn(-c2cccc(Cl)c2)c2c1CCN(c1cccc(N3C(=O)CC4CC43)c1)C2=O ; CCNC(=O)c1nn(-c2cccc(Cl)c2)c2c1CCN(c1cccc(N3C(=O)C[C@H]4C[C@H]43)c1)C2=O ; CCNC(=O)c1nn(-c2cccc(Cl)c2)c2c1CCN(c1cccc(N3C(=O)C[C@@H]4C[C@@H]43)c1)C2=O]

1-(3-Chlorophenyl)-N-ethyl-7-oxo-6-(3-(3-oxo-2-azabicyclo[3.1.0]hexan-2-yl)phenyl)-4,5,6,7-tetrahydro-1H-pyrazolo[3,4-c]pyridine-3-carboxamide (95 mg, 0.19 mmol) was dissolved to 12.5 mg/ml in EtOH and was purified by SFC (25:75 EtOH: CO₂ (0.2% v/v NH₃) on a Phenomenex Lux Cellulose-1 column. Combined fractions of each enantiomer were then evaporated and lyophilised to afford the arbitrarily assigned enantiomers:

### 1-(3-chlorophenyl)-N-ethyl-7-oxo-6-(3-((1R*,5R*)-3-oxo-2-azabicyclo[3.1.0]hexan-2-yl)phenyl)-4,5,6,7-tetrahydro-1H-pyrazolo[3,4-c]pyridine-3-carboxamide (22 mg, 23% yield, 98% ee)

[M+H]⁺=490.1

¹H NMR (DMSO-d6, 400 MHZ) δ 0.46 (1H, td, J=5.3, 2.3 Hz), 1.04-1.08 (1H, m), 1.11 (3H, t, J=7.2 Hz), 1.54-1.61 (1H, m), 2.46 (1H, d, J=18.2 Hz), 2.96 (1H, dd, J=18.2, 7.3 Hz), 3.21 (2H, t, J=6.6 Hz), 3.28 (2H, dd, J=7.3, 6.0 Hz), 3.54 (1H, t, J=6.0 Hz), 4.08 (2H, t, J=6.7 Hz), 7.13-7.17 (1H, m), 7.41 (1H, t, J=8.1 Hz), 7.46-7.57 (3H, m), 7.63 (1H, dt, J=6.8, 2.1 Hz), 7.69 (1H, t, J=2.1 Hz), 7.77-7.80 (1H, m), 8.46 (1H, t, J=5.9 Hz)

### 1-(3-chlorophenyl)-N-ethyl-7-oxo-6-(3-((1S*,5S*)-3-oxo-2-azabicyclo[3.1.0]hexan-2-yl)phenyl)-4,5,6,7-tetrahydro-1H-pyrazolo[3,4-c]pyridine-3-carboxamide (26 mg, 27% yield, 95.3% ee)

[M+H]⁺=490.1

¹H NMR (DMSO-d6, 400 MHZ) δ 0.46 (1H, td, J=5.2, 2.2 Hz), 1.03-1.08 (1H, m), 1.11 (3H, t, J=7.2 Hz), 1.53-1.63 (1H, m), 2.46 (1H, d, J=18.1 Hz), 2.96 (1H, dd, J=18.2, 7.3 Hz), 3.21 (2H, t, J=6.6 Hz), 3.28 (2H, dd, J=7.4, 6.1 Hz), 3.55 (1H, t, J=6.0 Hz), 4.08 (2H, t, J=6.6 Hz), 7.12-7.17 (1H, m), 7.41 (1H, t, J=8.1 Hz), 7.47-7.57 (3H, m), 7.63 (1H, dt, J=6.8, 2.1 Hz), 7.69 (1H, t, J=2.1 Hz), 7.79 (1H, t, J=2.0 Hz), 8.46 (1H, t, J=6.0 Hz)

### Example number 1423

### 1-(3-Chlorophenyl)-N-ethyl-6-(3-(4-methylpiperazin-1-yl)phenyl)-7-oxo-4,5,6,7-tetrahydro-1H-pyrazolo[3,4-c]pyridine-3-carboxamide

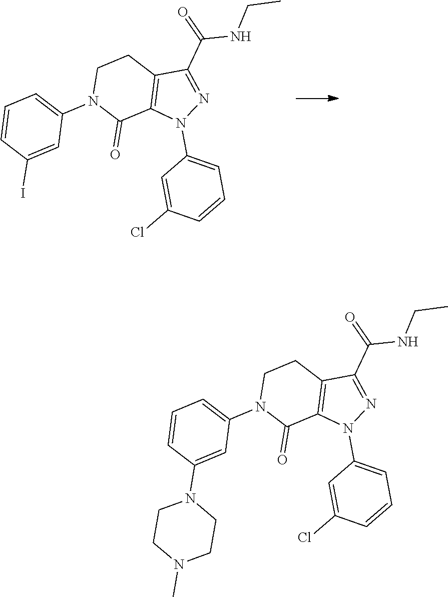 [SMILES: CCNC(=O)c1nn(-c2cccc(Cl)c2)c2c1CCN(c1cccc(I)c1)C2=O ; CCNC(=O)c1nn(-c2cccc(Cl)c2)c2c1CCN(c1cccc(N3CCN(C)CC3)c1)C2=O]

1-(3-Chlorophenyl)-N-ethyl-6-(3-iodophenyl)-7-oxo-4,5-dihydropyrazolo[5,4-c]pyridine-3-carboxamide (50 mg, 0.08 mmol), 1-methyl piperazine (12 mg, 0.12 mmol) and NaOtBu (22 mg, 0.23 mmol) were suspended in 1,4-dioxane (2 mL). The mixture was purged with N₂ (g) for 5 min before RuPhos (7 mg, 0.02 mmol) and RuPhos Pd G4 (7 mg, 0.01 mmol) were added. The mixture was purged with N₂ (g) for an additional 5 min and heated at 110° C. for 18 h. The mixture was cooled to rt, diluted with EtOAc (30 mL) and water (30 mL). The aqueous layer was re-extracted with EtOAc (3×20 mL) and the combined organics were washed with brine (10 mL), filtered and concentrated. Purification was performed by automated prep HPLC (mass directed 5-95% MeCN in water with an acidic mobile phase of 2.5% formic acid in water). The product was dissolved in MeOH (5 mL), passed through a solid supported HCO₃ resin cartridge, and flushed with MeOH (3×5 mL) to afford the product (19 mg, 50% yield).

[M+H]⁺=493.4

¹H NMR (DMSO-d6, 400 MHZ) δ 1.11 (3H, t, J=7.2 Hz), 2.21 (3H, s), 2.43 (4H, t, J=5.0 Hz), 3.12 (4H, dd, J=5.1, 5.1 Hz), 3.18 (2H, t, J=6.6 Hz), 3.23-3.30 (2H, m), 4.02 (2H, t, J=6.6 Hz), 6.73-6.76 (1H, m), 6.83 (1H, dd, J=8.2, 2.4 Hz), 6.90 (1H, dd, J=2.2, 2.2 Hz), 7.21 (1H, dd, J=8.1, 8.1 Hz), 7.46-7.56 (2H, m), 7.60 -7.63 (1H, m), 7.74-7.80 (1H, m), 8.45 (1H, t, J=5.9 Hz)

### Example number 2038

### 3-(3-(Methoxymethyl)-6-(2-methyl-1-oxo-1,2,3,4-tetrahydroisoquinolin-7-yl)-7-oxo-4,5,6,7-tetrahydro-1H-pyrazolo[3,4-c]pyridin-1-yl)-2-methylbenzonitrile

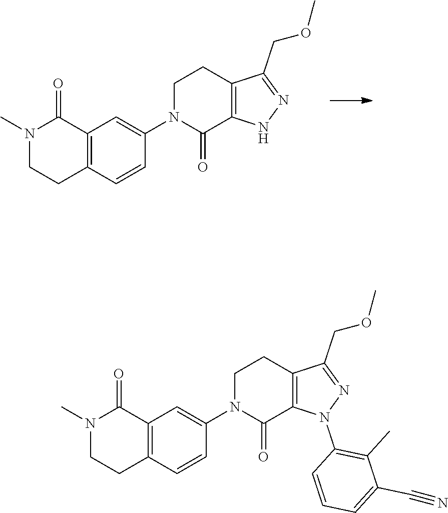

3-lodo-2-methyl-benzonitrile (97.1 mg, 0.280 mmol) was added to a mixture of 7-[3-(methoxymethyl)-7-oxo-4,5-dihydro-1H-pyrazolo[3,4-c]pyridin-6-yl]-2-methyl-3,4-dihydroisoquinolin-1-one (40 mg, 0.112 mmol), K₃PO₄ (54 mg, 0.249 mmol), BFMO (9.7 mg, 0.038 mmol) and Cu₂O (8.6 mg, 0.059 mmol) in DMSO (2 mL) at rt. The mixture was sealed and stirred at 130° C. for 16 h. The mixture was filtered and concentrated. Purification by automated prep HPLC (UV directed, 30-40% MeCN in 10 mM (NH₄) HCO₃ (aq) over 8 min) and lyophilisation afforded the product (25 mg, 49% yield).

[M+H]⁺=456.7

¹H NMR (DMSO-d6, 500 MHz) δ 2.19 (3H, s), 2.96 (2H, t, J=6.6 Hz), 3.02 (3H, s), 3.04 (2H, d, J=6.7 Hz), 3.32 (3H, s), 3.53 (2H, t, J=7.1, 6.2 Hz), 4.07 (2H, t, J=6.8 Hz), 4.52 (2H, s), 7.29 (1H, d, J=8.1 Hz), 7.41 (1H, dd, J=8.1, 2.4 Hz), 7.49 (1H, t, J=7.9), 7.67 (1H, dd, J=8.0, 1.3 Hz), 7.77 (1H, d, J=2.3 Hz), 7.91 (1H, dd, J=7.8, 1.3 Hz)

### Examples

TABLE 5
Compound Names

| Example | |
|---|---|
| Number | Name |
| 1001 | N-benzyl-1-(3-chlorophenyl)-7-oxo-6-(3-(piperidin-1-ylmethyl)phenyl)-4,5,6,7- |
| | tetrahydro-1H-pyrazolo[3,4-c]pyridine-3-carboxamide |
| 1002 | 1-(3-chlorophenyl)-N-(cyclopropylmethyl)-7-oxo-6-(3-(piperidin-1-ylmethyl)phenyl)- |
| | 4,5,6,7-tetrahydro-1H-pyrazolo[3,4-c]pyridine-3-carboxamide |
| 1003 | 1-(3-chlorophenyl)-7-oxo-6-(3-(piperidin-1-ylmethyl)phenyl)-N-(pyridin-3-ylmethyl)- |
| | 4,5,6,7-tetrahydro-1H-pyrazolo[3,4-c]pyridine-3-carboxamide |
| 1004 | N-benzyl-1-(3-chlorophenyl)-N-methyl-7-oxo-6-(3-(piperidin-1-ylmethyl)phenyl)- |
| | 4,5,6,7-tetrahydro-1H-pyrazolo[3,4-c]pyridine-3-carboxamide |
| 1005 | N-benzyl-1-(3-chlorophenyl)-6-(2-methyl-1,2,3,4-tetrahydroisoquinolin-7-yl)-7-oxo- |
| | 4,5,6,7-tetrahydro-1H-pyrazolo[3,4-c]pyridine-3-carboxamide |
| 1006 | benzyl 1-(3-chlorophenyl)-7-oxo-6-(3-(piperidin-1-ylmethyl)phenyl)-4,5,6,7- |
| | tetrahydro-1H-pyrazolo[3,4-c]pyridine-3-carboxylate |
| 1007 | ethyl 1-(3-chlorophenyl)-7-oxo-6-(3-(piperidin-1-ylmethyl)phenyl)-4,5,6,7-tetrahydro- |
| | 1H-pyrazolo[3,4-c]pyridine-3-carboxylate |
| 1008 | (S)-1-(3-chlorophenyl)-N-(2-hydroxy-1-phenylethyl)-7-oxo-6-(3-(piperidin-1- |
| | ylmethyl)phenyl)-4,5,6,7-tetrahydro-1H-pyrazolo[3,4-c]pyridine-3-carboxamide |
| 1009 | 1-(3-chlorophenyl)-6-(3-(piperidin-1-ylmethyl)phenyl)-3-(pyrrolidine-1-carbonyl)- |
| | 1,4,5,6-tetrahydro-7H-pyrazolo[3,4-c]pyridin-7-one |
| 1010 | 1-(3-chlorophenyl)-N-cyclohexyl-7-oxo-6-(3-(piperidin-1-ylmethyl)phenyl)-4,5,6,7- |
| | tetrahydro-1H-pyrazolo[3,4-c]pyridine-3-carboxamide |
| 1011 | 1-(3-chlorophenyl)-N-ethyl-7-oxo-6-(3-(piperidin-1-ylmethyl)phenyl)-4,5,6,7- |
| | tetrahydro-1H-pyrazolo[3,4-c]pyridine-3-carboxamide |
| 1012 | 1-(3-chlorophenyl)-N-(cyclopropylmethyl)-6-(2-methyl-1,2,3,4-tetrahydroisoquinolin- |
| | 7-yl)-7-oxo-4,5,6,7-tetrahydro-1H-pyrazolo[3,4-c]pyridine-3-carboxamide |
| 1013 | ethyl 1-(3-chlorophenyl)-6-(2-methyl-1,2,3,4-tetrahydroisoquinolin-7-yl)-7-oxo- |
| | 4,5,6,7-tetrahydro-1H-pyrazolo[3,4-c]pyridine-3-carboxylate |
| 1014 | 1-(3-chlorophenyl)-N-ethyl-6-(2-methyl-1,2,3,4-tetrahydroisoquinolin-7-yl)-7-oxo- |
| | 4,5,6,7-tetrahydro-1H-pyrazolo[3,4-c]pyridine-3-carboxamide |
| 1015 | 1-(3-chlorophenyl)-6-(2-methyl-1,2,3,4-tetrahydroisoquinolin-7-yl)-7-oxo-N-(pyridin- |
| | 3-ylmethyl)-4,5,6,7-tetrahydro-1H-pyrazolo[3,4-c]pyridine-3-carboxamide |
| 1016 | N-benzyl-1-(3-chlorophenyl)-6-((1-methylpiperidin-4-yl)methyl)-7-oxo-4,5,6,7- |
| | tetrahydro-1H-pyrazolo[3,4-c]pyridine-3-carboxamide |
| 1017 | N-benzyl-1-(3-chlorophenyl)-7-oxo-6-(3-(pyrrolidin-1-ylmethyl)phenyl)-4,5,6,7- |
| | tetrahydro-1H-pyrazolo[3,4-c]pyridine-3-carboxamide |
| 1018 | N-benzyl-1-(3-chlorophenyl)-6-(3-(morpholinomethyl)phenyl)-7-oxo-4,5,6,7- |
| | tetrahydro-1H-pyrazolo[3,4-c]pyridine-3-carboxamide |
| 1019 | N-benzyl-1-(3-chlorophenyl)-6-(2-(cyclopropylmethyl)-1,2,3,4-tetrahydroisoquinolin- |
| | 7-yl)-7-oxo-4,5,6,7-tetrahydro-1H-pyrazolo[3,4-c]pyridine-3-carboxamide |
| 1020 | N-benzyl-1-(3-chlorophenyl)-6-(2-(cyclopentylmethyl)-1,2,3,4-tetrahydroisoquinolin- |
| | 7-yl)-7-oxo-4,5,6,7-tetrahydro-1H-pyrazolo[3,4-c]pyridine-3-carboxamide |
| 1021 | N-benzyl-1-(3-chlorophenyl)-6-(2-ethyl-1,2,3,4-tetrahydroisoquinolin-7-yl)-7-oxo- |
| | 4,5,6,7-tetrahydro-1H-pyrazolo[3,4-c]pyridine-3-carboxamide |
| 1022 | N-benzyl-1-(3-chlorophenyl)-6-(2-isopropyl-1,2,3,4-tetrahydroisoquinolin-7-yl)-7-oxo- |
| | 4,5,6,7-tetrahydro-1H-pyrazolo[3,4-c]pyridine-3-carboxamide |
| 1023 | 1-(3-chlorophenyl)-N-(cyclohexylmethyl)-6-(2-methyl-1,2,3,4-tetrahydroisoquinolin-7- |
| | yl)-7-oxo-4,5,6,7-tetrahydro-1H-pyrazolo[3,4-c]pyridine-3-carboxamide |
| 1024 | (S)-1-(3-chlorophenyl)-N-(2-hydroxy-1-phenylethyl)-6-(2-methyl-1,2,3,4- |
| | tetrahydroisoquinolin-7-yl)-7-oxo-4,5,6,7-tetrahydro-1H-pyrazolo[3,4-c]pyridine-3- |
| | carboxamide |
| 1025 | 1-(3-chlorophenyl)-6-(2-methyl-1,2,3,4-tetrahydroisoquinolin-7-yl)-7-oxo-N- |
| | ((tetrahydrofuran-2-yl)methyl)-4,5,6,7-tetrahydro-1H-pyrazolo[3,4-c]pyridine-3- |
| | carboxamide |
| 1026 | 1-(3-chlorophenyl)-6-(2-methyl-1,2,3,4-tetrahydroisoquinolin-7-yl)-7-oxo-N- |
| | ((tetrahydrofuran-3-yl)methyl)-4,5,6,7-tetrahydro-1H-pyrazolo[3,4-c]pyridine-3- |
| | carboxamide |
| 1027 | 1-(3-chlorophenyl)-6-(2-methyl-1,2,3,4-tetrahydroisoquinolin-7-yl)-7-oxo-N- |
| | ((tetrahydro-2H-pyran-4-yl)methyl)-4,5,6,7-tetrahydro-1H-pyrazolo[3,4-c]pyridine-3- |
| | carboxamide |
| 1028 | N-benzyl-1-(3-chlorophenyl)-6-(2-methylisoindolin-5-yl)-7-oxo-4,5,6,7-tetrahydro-1H- |
| | pyrazolo[3,4-c]pyridine-3-carboxamide |
| 1029 | N-benzyl-1-(3-chlorophenyl)-6-(2-cyclopropyl-1,2,3,4-tetrahydroisoquinolin-7-yl)-7- |
| | oxo-4,5,6,7-tetrahydro-1H-pyrazolo[3,4-c]pyridine-3-carboxamide |
| 1030 | N-benzyl-1-(3-chlorophenyl)-6-(2-methyl-2,3,4,5-tetrahydro-1H-benzo[c]azepin-8-yl)- |
| | 7-oxo-4,5,6,7-tetrahydro-1H-pyrazolo[3,4-c]pyridine-3-carboxamide |
| 1031 | 1-(3-chlorophenyl)-N-(cyclopropylmethyl)-6-(2-ethyl-1,2,3,4-tetrahydroisoquinolin-7- |
| | yl)-7-oxo-4,5,6,7-tetrahydro-1H-pyrazolo[3,4-c]pyridine-3-carboxamide |
| 1032 | 1-(3-chlorophenyl)-N-(cyclopropylmethyl)-6-(2-(oxetan-3-yl)-1,2,3,4- |
| | tetrahydroisoquinolin-7-yl)-7-oxo-4,5,6,7-tetrahydro-1H-pyrazolo[3,4-c]pyridine-3- |
| | carboxamide |
| 1033 | 6-(2-(2-amino-2-oxoethyl)-1,2,3,4-tetrahydroisoquinolin-7-yl)-1-(3-chlorophenyl)-N- |
| | (cyclopropylmethyl)-7-oxo-4,5,6,7-tetrahydro-1H-pyrazolo[3,4-c]pyridine-3- |
| | carboxamide |
| 1034 | 1-(3-chlorophenyl)-N-(cyclopropylmethyl)-6-(7-methyl-5,6,7,8-tetrahydro-1,7- |
| | naphthyridin-2-yl)-7-oxo-4,5,6,7-tetrahydro-1H-pyrazolo[3,4-c]pyridine-3- |
| | carboxamide |
| 1035 | 1-(3-chlorophenyl)-N-(cyclopropylmethyl)-6-(2-isopropyl-1,2,3,4- |
| | tetrahydroisoquinolin-7-yl)-7-oxo-4,5,6,7-tetrahydro-1H-pyrazolo[3,4-c]pyridine-3- |
| | carboxamide |
| 1036 | 1-(3-chlorophenyl)-N-(cyclopropylmethyl)-6-(2-(cyclopropylmethyl)-1,2,3,4- |
| | tetrahydroisoquinolin-7-yl)-7-oxo-4,5,6,7-tetrahydro-1H-pyrazolo[3,4-c]pyridine-3- |
| | carboxamide |
| 1037 | 1-(3-chlorophenyl)-N-(cyclopropylmethyl)-6-(2-(2-methoxyethyl)-1,2,3,4- |
| | tetrahydroisoquinolin-7-yl)-7-oxo-4,5,6,7-tetrahydro-1H-pyrazolo[3,4-c]pyridine-3- |
| | carboxamide |
| 1038 | N-benzyl-1-(3-chlorophenyl)-6-(3-((3-methylpyrrolidin-1-yl)methyl)phenyl)-7-oxo- |
| | 4,5,6,7-tetrahydro-1H-pyrazolo[3,4-c]pyridine-3-carboxamide |
| 1039 | N-benzyl-1-(3-chlorophenyl)-6-(3-((2-methylpyrrolidin-1-yl)methyl)phenyl)-7-oxo- |
| | 4,5,6,7-tetrahydro-1H-pyrazolo[3,4-c]pyridine-3-carboxamide |
| 1040 | 1-(3-chlorophenyl)-N-(cyclopropylmethyl)-6-(6-methyl-5,6,7,8-tetrahydro-1,6- |
| | naphthyridin-3-yl)-7-oxo-4,5,6,7-tetrahydro-1H-pyrazolo[3,4-c]pyridine-3- |
| | carboxamide |
| 1041 | 1-(3-chlorophenyl)-6-(2-methyl-1,2,3,4-tetrahydroisoquinolin-7-yl)-N-(oxetan-3-yl)-7- |
| | oxo-4,5,6,7-tetrahydro-1H-pyrazolo[3,4-c]pyridine-3-carboxamide |
| 1042 | 1-(3-chlorophenyl)-6-(2-methyl-1,2,3,4-tetrahydroisoquinolin-7-yl)-7-oxo-N- |
| | (tetrahydrofuran-3-yl)-4,5,6,7-tetrahydro-1H-pyrazolo[3,4-c]pyridine-3-carboxamide |
| 1043 | 1-(3-chlorophenyl)-6-(2-methyl-1,2,3,4-tetrahydroisoquinolin-7-yl)-7-oxo-N- |
| | (tetrahydro-2H-pyran-4-yl)-4,5,6,7-tetrahydro-1H-pyrazolo[3,4-c]pyridine-3- |
| | carboxamide |
| 1044 | 1-(3-chlorophenyl)-N-cyclopentyl-6-(2-methyl-1,2,3,4-tetrahydroisoquinolin-7-yl)-7- |
| | oxo-4,5,6,7-tetrahydro-1H-pyrazolo[3,4-c]pyridine-3-carboxamide |
| 1045 | 1-(3-chlorophenyl)-6-(2-methyl-1,2,3,4-tetrahydroisoquinolin-7-yl)-N-(oxetan-2- |
| | ylmethyl)-7-oxo-4,5,6,7-tetrahydro-1H-pyrazolo[3,4-c]pyridine-3-carboxamide |
| 1046 | 1-(3-chlorophenyl)-N-((5-(hydroxymethyl)-2-oxabicyclo[3.1.1]heptan-1-yl)methyl)-6- |
| | (2-methyl-1,2,3,4-tetrahydroisoquinolin-7-yl)-7-oxo-4,5,6,7-tetrahydro-1H- |
| | pyrazolo[3,4-c]pyridine-3-carboxamide |
| 1047 | 1-(3-chlorophenyl)-6-(2-methyl-1,2,3,4-tetrahydroisoquinolin-7-yl)-N-(oxetan-3- |
| | ylmethyl)-7-oxo-4,5,6,7-tetrahydro-1H-pyrazolo[3,4-c]pyridine-3-carboxamide |
| 1048 | 1-(3-chlorophenyl)-N-(cyclopropylmethyl)-6-(2,5-dimethyl-1,2,3,4- |
| | tetrahydroisoquinolin-7-yl)-7-oxo-4,5,6,7-tetrahydro-1H-pyrazolo[3,4-c]pyridine-3- |
| | carboxamide |
| 1049 | 1-(3-chlorophenyl)-N-((1-(hydroxymethyl)cyclobutyl)methyl)-6-(2-methyl-1,2,3,4- |
| | tetrahydroisoquinolin-7-yl)-7-oxo-4,5,6,7-tetrahydro-1H-pyrazolo[3,4-c]pyridine-3- |
| | carboxamide |
| 1050 | 1-(3-chlorophenyl)-N-((1-(hydroxymethyl)-2-oxabicyclo[2.1.1]hexan-4-yl)methyl)-6-(2- |
| | methyl-1,2,3,4-tetrahydroisoquinolin-7-yl)-7-oxo-4,5,6,7-tetrahydro-1H-pyrazolo[3,4- |
| | c]pyridine-3-carboxamide |
| 1051 | (S)-1-(3-chlorophenyl)-6-(2-ethyl-1,2,3,4-tetrahydroisoquinolin-7-yl)-N-(2-hydroxy-1- |
| | phenylethyl)-7-oxo-4,5,6,7-tetrahydro-1H-pyrazolo[3,4-c]pyridine-3-carboxamide |
| 1052 | 1-(3-chlorophenyl)-N-(cyclopropylmethyl)-7-oxo-6-(2-propyl-1,2,3,4- |
| | tetrahydroisoquinolin-7-yl)-4,5,6,7-tetrahydro-1H-pyrazolo[3,4-c]pyridine-3- |
| | carboxamide |
| 1053 | 1-(3-chlorophenyl)-N-(cyclopropylmethyl)-6-(2-(3-methoxypropyl)-1,2,3,4- |
| | tetrahydroisoquinolin-7-yl)-7-oxo-4,5,6,7-tetrahydro-1H-pyrazolo[3,4-c]pyridine-3- |
| | carboxamide |
| 1054 | 1-(3-chlorophenyl)-N-(cyclopropylmethyl)-6-(2-ethyl-6-methyl-1,2,3,4- |
| | tetrahydroisoquinolin-7-yl)-7-oxo-4,5,6,7-tetrahydro-1H-pyrazolo[3,4-c]pyridine-3- |
| | carboxamide |
| 1055 | 1-(3-chlorophenyl)-N-(cyclopropylmethyl)-6-(2-(2-hydroxyethyl)-1,2,3,4- |
| | tetrahydroisoquinolin-7-yl)-7-oxo-4,5,6,7-tetrahydro-1H-pyrazolo[3,4-c]pyridine-3- |
| | carboxamide |
| 1056 | 1-(3-chlorophenyl)-N-(cyclopropylmethyl)-6-(7-ethyl-5,6,7,8-tetrahydropyrido[3,4- |
| | d]pyrimidin-2-yl)-7-oxo-4,5,6,7-tetrahydro-1H-pyrazolo[3,4-c]pyridine-3-carboxamide |
| 1057 | 1-(3-chlorophenyl)-6-(2-methyl-1,2,3,4-tetrahydroisoquinolin-7-yl)-7-oxo-N-(2- |
| | oxopiperidin-4-yl)-4,5,6,7-tetrahydro-1H-pyrazolo[3,4-c]pyridine-3-carboxamide |
| 1058 | 1-(3-chlorophenyl)-N-(2-cyclopropyl-2-hydroxyethyl)-6-(2-methyl-1,2,3,4- |
| | tetrahydroisoquinolin-7-yl)-7-oxo-4,5,6,7-tetrahydro-1H-pyrazolo[3,4-c]pyridine-3- |
| | carboxamide |
| 1059 | 6-(2-(sec-butyl)-1,2,3,4-tetrahydroisoquinolin-7-yl)-1-(3-chlorophenyl)-N- |
| | (cyclopropylmethyl)-7-oxo-4,5,6,7-tetrahydro-1H-pyrazolo[3,4-c]pyridine-3- |
| | carboxamide |
| 1060 | 1-(3-chlorophenyl)-N-(cyclopropylmethyl)-6-(2-(1-methoxypropan-2-yl)-1,2,3,4- |
| | tetrahydroisoquinolin-7-yl)-7-oxo-4,5,6,7-tetrahydro-1H-pyrazolo[3,4-c]pyridine-3- |
| | carboxamide |
| 1061 | 1-(3-chlorophenyl)-N-(cyclopropylmethyl)-6-(2-(2-ethoxyethyl)-1,2,3,4- |
| | tetrahydroisoquinolin-7-yl)-7-oxo-4,5,6,7-tetrahydro-1H-pyrazolo[3,4-c]pyridine-3- |
| | carboxamide |
| 1063 | (R)-1-(3-chlorophenyl)-N-(2-hydroxy-1-phenylethyl)-6-(2-methyl-1,2,3,4- |
| | tetrahydroisoquinolin-7-yl)-7-oxo-4,5,6,7-tetrahydro-1H-pyrazolo[3,4-c]pyridine-3- |
| | carboxamide |
| 1064 | 1-(3-chlorophenyl)-6-(2-methyl-1,2,3,4-tetrahydroisoquinolin-7-yl)-N-(4- |
| | methyltetrahydro-2H-pyran-4-yl)-7-oxo-4,5,6,7-tetrahydro-1H-pyrazolo[3,4- |
| | c]pyridine-3-carboxamide |
| 1065 | 1-(3-chlorophenyl)-6-(2-methyl-1,2,3,4-tetrahydroisoquinolin-7-yl)-N-((3-methyl-1H- |
| | pyrazol-5-yl)methyl)-7-oxo-4,5,6,7-tetrahydro-1H-pyrazolo[3,4-c]pyridine-3- |
| | carboxamide |
| 1066 | 1-(3-chlorophenyl)-3-(methoxymethyl)-6-(2-methyl-1,2,3,4-tetrahydroisoquinolin-7- |
| | yl)-1,4,5,6-tetrahydro-7H-pyrazolo[3,4-c]pyridin-7-one |
| 1067 | 1-(3-chlorophenyl)-6-(2-methyl-1,2,3,4-tetrahydroisoquinolin-7-yl)-3-(morpholine-4- |
| | carbonyl)-1,4,5,6-tetrahydro-7H-pyrazolo[3,4-c]pyridin-7-one |
| 1068 | 1-(3-chlorophenyl)-N-(furan-3-ylmethyl)-6-(2-methyl-1,2,3,4-tetrahydroisoquinolin-7- |
| | yl)-7-oxo-4,5,6,7-tetrahydro-1H-pyrazolo[3,4-c]pyridine-3-carboxamide |
| 1069 | 1-(3-chlorophenyl)-6-(2-(2-hydroxyethyl)-1,2,3,4-tetrahydroisoquinolin-7-yl)-3- |
| | (methoxymethyl)-1,4,5,6-tetrahydro-7H-pyrazolo[3,4-c]pyridin-7-one |
| 1070 | (S)-1-(3-chlorophenyl)-N-(cyclopropylmethyl)-6-(2-(2-hydroxypropyl)-1,2,3,4- |
| | tetrahydroisoquinolin-7-yl)-7-oxo-4,5,6,7-tetrahydro-1H-pyrazolo[3,4-c]pyridine-3- |
| | carboxamide |
| 1071 | 1-(3-chlorophenyl)-N-(cyclopropylmethyl)-7-oxo-6-(2-(2,2,2-trifluoroethyl)-1,2,3,4- |
| | tetrahydroisoquinolin-7-yl)-4,5,6,7-tetrahydro-1H-pyrazolo[3,4-c]pyridine-3- |
| | carboxamide |
| 1072 | 1-(3-chlorophenyl)-N-(cyclopropylmethyl)-7-oxo-6-(3-(pyrrolidin-1-ylmethyl)phenyl)- |
| | 4,5,6,7-tetrahydro-1H-pyrazolo[3,4-c]pyridine-3-carboxamide |
| 1073 | 6-(3-((3-azabicyclo[3.1.0]hexan-3-yl)methyl)phenyl)-1-(3-chlorophenyl)-N- |
| | (cyclopropylmethyl)-7-oxo-4,5,6,7-tetrahydro-1H-pyrazolo[3,4-c]pyridine-3- |
| | carboxamide |
| 1074 | 1-(3-chlorophenyl)-N-(cyclopropylmethyl)-7-oxo-6-(1,2,3,4-tetrahydroisoquinolin-7- |
| | yl)-4,5,6,7-tetrahydro-1H-pyrazolo[3,4-c]pyridine-3-carboxamide |
| 1075 | N-(7-oxabicyclo[2.2.1]heptan-2-yl)-1-(3-chlorophenyl)-6-(2-methyl-1,2,3,4- |
| | tetrahydroisoquinolin-7-yl)-7-oxo-4,5,6,7-tetrahydro-1H-pyrazolo[3,4-c]pyridine-3- |
| | carboxamide |
| 1076 | 1-(3-chlorophenyl)-6-(2-(2-hydroxyethyl)-1,2,3,4-tetrahydroisoquinolin-7-yl)-7-oxo-N- |
| | (tetrahydro-2H-pyran-4-yl)-4,5,6,7-tetrahydro-1H-pyrazolo[3,4-c]pyridine-3- |
| | carboxamide |
| 1077 | 1-(3-chlorophenyl)-6-(2-(2-hydroxyethyl)-1,2,3,4-tetrahydroisoquinolin-7-yl)-N-((3- |
| | methyl-1H-pyrazol-5-yl)methyl)-7-oxo-4,5,6,7-tetrahydro-1H-pyrazolo[3,4-c]pyridine- |
| | 3-carboxamide |
| 1078 | 1-(3-chlorophenyl)-6-(2-(2-hydroxyethyl)-1,2,3,4-tetrahydroisoquinolin-7-yl)-7-oxo-N- |
| | (pyridin-3-ylmethyl)-4,5,6,7-tetrahydro-1H-pyrazolo[3,4-c]pyridine-3-carboxamide |
| 1079 | 1-(3-chlorophenyl)-N-(cyclopropylmethyl)-6-(2-ethyl-6-fluoro-1,2,3,4- |
| | tetrahydroisoquinolin-7-yl)-7-oxo-4,5,6,7-tetrahydro-1H-pyrazolo[3,4-c]pyridine-3- |
| | carboxamide |
| 1080 | 1-(3-chlorophenyl)-N-(cyclopropylmethyl)-6-(2-(3-fluoropropyl)-1,2,3,4- |
| | tetrahydroisoquinolin-7-yl)-7-oxo-4,5,6,7-tetrahydro-1H-pyrazolo[3,4-c]pyridine-3- |
| | carboxamide |
| 1081 | 6-(2-(2-acetamidoethyl)-1,2,3,4-tetrahydroisoquinolin-7-yl)-1-(3-chlorophenyl)-N- |
| | (cyclopropylmethyl)-7-oxo-4,5,6,7-tetrahydro-1H-pyrazolo[3,4-c]pyridine-3- |
| | carboxamide |
| 1082 | 1-(3-chlorophenyl)-N-(cyclopropylmethyl)-6-(2-ethyl-4,4-dimethyl-1,2,3,4- |
| | tetrahydroisoquinolin-7-yl)-7-oxo-4,5,6,7-tetrahydro-1H-pyrazolo[3,4-c]pyridine-3- |
| | carboxamide |
| 1083 | 1-(3-chlorophenyl)-N-(cyclopropylmethyl)-6-(2-(2-(methylsulfonamido)ethyl)-1,2,3,4- |
| | tetrahydroisoquinolin-7-yl)-7-oxo-4,5,6,7-tetrahydro-1H-pyrazolo[3,4-c]pyridine-3- |
| | carboxamide |
| 1084 | 1-(3-chlorophenyl)-N-(cyclopropylmethyl)-6-(2-(2-(3-methylureido)ethyl)-1,2,3,4- |
| | tetrahydroisoquinolin-7-yl)-7-oxo-4,5,6,7-tetrahydro-1H-pyrazolo[3,4-c]pyridine-3- |
| | carboxamide |
| 1085 | 1-(3-chlorophenyl)-6-(2-ethyl-1,2,3,4-tetrahydroisoquinolin-7-yl)-7-oxo-N-(tetrahydro- |
| | 2H-pyran-4-yl)-4,5,6,7-tetrahydro-1H-pyrazolo[3,4-c]pyridine-3-carboxamide |
| 1086 | 1-(3-chlorophenyl)-6-(2-ethyl-1,2,3,4-tetrahydroisoquinolin-7-yl)-N-((3-methyl-1H- |
| | pyrazol-5-yl)methyl)-7-oxo-4,5,6,7-tetrahydro-1H-pyrazolo[3,4-c]pyridine-3- |
| | carboxamide |
| 1087 | 1-(3-chlorophenyl)-N-(cyclopropylmethyl)-6-(2-(3-hydroxypropyl)-1,2,3,4- |
| | tetrahydroisoquinolin-7-yl)-7-oxo-4,5,6,7-tetrahydro-1H-pyrazolo[3,4-c]pyridine-3- |
| | carboxamide |
| 1088 | 1-(3-chlorophenyl)-6-(2-(2-hydroxyethyl)-1,2,3,4-tetrahydroisoquinolin-7-yl)-N-((1- |
| | methyl-1H-pyrazol-5-yl)methyl)-7-oxo-4,5,6,7-tetrahydro-1H-pyrazolo[3,4-c]pyridine- |
| | 3-carboxamide |
| 1089 | N-((1H-pyrazol-5-yl)methyl)-1-(3-chlorophenyl)-6-(2-(2-hydroxyethyl)-1,2,3,4- |
| | tetrahydroisoquinolin-7-yl)-7-oxo-4,5,6,7-tetrahydro-1H-pyrazolo[3,4-c]pyridine-3- |
| | carboxamide |
| 1090 | (S)-1-(3-chlorophenyl)-6-(2-ethyl-1,2,3,4-tetrahydroisoquinolin-7-yl)-7-oxo-N- |
| | (tetrahydrofuran-3-yl)-4,5,6,7-tetrahydro-1H-pyrazolo[3,4-c]pyridine-3-carboxamide |
| 1091 | (R)-1-(3-chlorophenyl)-6-(2-ethyl-1,2,3,4-tetrahydroisoquinolin-7-yl)-7-oxo-N- |
| | (tetrahydrofuran-3-yl)-4,5,6,7-tetrahydro-1H-pyrazolo[3,4-c]pyridine-3-carboxamide |
| 1092 | 1-(3-chlorophenyl)-N-(4,4-dimethyltetrahydrofuran-3-yl)-6-(2-ethyl-1,2,3,4- |
| | tetrahydroisoquinolin-7-yl)-7-oxo-4,5,6,7-tetrahydro-1H-pyrazolo[3,4-c]pyridine-3- |
| | carboxamide |
| 1093 | N-(3-oxabicyclo[3.1.0]hexan-6-yl)-1-(3-chlorophenyl)-6-(2-ethyl-1,2,3,4- |
| | tetrahydroisoquinolin-7-yl)-7-oxo-4,5,6,7-tetrahydro-1H-pyrazolo[3,4-c]pyridine-3- |
| | carboxamide |
| 1094 | methyl 2-(7-(1-(3-chlorophenyl)-3-((cyclopropylmethyl)carbamoyl)-7-oxo-1,4,5,7- |
| | tetrahydro-6H-pyrazolo[3,4-c]pyridin-6-yl)-3,4-dihydroisoquinolin-2(1H)-yl)acetate |
| 1095 | 2-(7-(1-(3-chlorophenyl)-3-((cyclopropylmethyl)carbamoyl)-7-oxo-1,4,5,7-tetrahydro- |
| | 6H-pyrazolo[3,4-c]pyridin-6-yl)-3,4-dihydroisoquinolin-2(1H)-yl)acetic acid |
| 1096 | 1-(3-chlorophenyl)-N-(cyclopropylmethyl)-6-(2-(2-hydroxy-2-methylpropyl)-1,2,3,4- |
| | tetrahydroisoquinolin-7-yl)-7-oxo-4,5,6,7-tetrahydro-1H-pyrazolo[3,4-c]pyridine-3- |
| | carboxamide |
| 1097 | 1-(3-chlorophenyl)-N-(cyclopropylmethyl)-6-(2-((2,2-difluorocyclopropyl)methyl)- |
| | 1,2,3,4-tetrahydroisoquinolin-7-yl)-7-oxo-4,5,6,7-tetrahydro-1H-pyrazolo[3,4- |
| | c]pyridine-3-carboxamide |
| 1098 | 6-(2-(2-aminoethyl)-1,2,3,4-tetrahydroisoquinolin-7-yl)-1-(3-chlorophenyl)-N- |
| | (cyclopropylmethyl)-7-oxo-4,5,6,7-tetrahydro-1H-pyrazolo[3,4-c]pyridine-3- |
| | carboxamide |
| 1099 | 1-(3-chlorophenyl)-N-(cyclopropylmethyl)-6-(2′-ethyl-2′,3′-dihydro-1′H- |
| | spiro[cyclopropane-1,4′-isoquinolin]-7′-yl)-7-oxo-4,5,6,7-tetrahydro-1H-pyrazolo[3,4- |
| | c]pyridine-3-carboxamide |
| 1100 | 1-(3-chlorophenyl)-6-(2-(cyanomethyl)-1,2,3,4-tetrahydroisoquinolin-7-yl)-N- |
| | (cyclopropylmethyl)-7-oxo-4,5,6,7-tetrahydro-1H-pyrazolo[3,4-c]pyridine-3- |
| | carboxamide |
| 1101 | (R)-1-(3-chlorophenyl)-N-(cyclopropylmethyl)-6-(2-(2-hydroxypropyl)-1,2,3,4- |
| | tetrahydroisoquinolin-7-yl)-7-oxo-4,5,6,7-tetrahydro-1H-pyrazolo[3,4-c]pyridine-3- |
| | carboxamide |
| 1102 | 1-(3-chlorophenyl)-6-(2-(cyclopentylmethyl)-1,2,3,4-tetrahydroisoquinolin-7-yl)-N- |
| | (cyclopropylmethyl)-7-oxo-4,5,6,7-tetrahydro-1H-pyrazolo[3,4-c]pyridine-3- |
| | carboxamide |
| 1103 | 1-(3-chlorophenyl)-6-(2-cyclobutyl-1,2,3,4-tetrahydroisoquinolin-7-yl)-N- |
| | (cyclopropylmethyl)-7-oxo-4,5,6,7-tetrahydro-1H-pyrazolo[3,4-c]pyridine-3- |
| | carboxamide |
| 1104 | 6-(2-((1H-pyrazol-5-yl)methyl)-1,2,3,4-tetrahydroisoquinolin-7-yl)-1-(3-chlorophenyl)- |
| | N-(cyclopropylmethyl)-7-oxo-4,5,6,7-tetrahydro-1H-pyrazolo[3,4-c]pyridine-3- |
| | carboxamide |
| 1105 | 1-(3-chlorophenyl)-N-(furan-3-ylmethyl)-6-(2-(2-hydroxyethyl)-1,2,3,4- |
| | tetrahydroisoquinolin-7-yl)-7-oxo-4,5,6,7-tetrahydro-1H-pyrazolo[3,4-c]pyridine-3- |
| | carboxamide |
| 1106 | 1-(3-chlorophenyl)-6-(2-(2-hydroxyethyl)-1,2,3,4-tetrahydroisoquinolin-7-yl)-7-oxo-N- |
| | ((tetrahydrofuran-3-yl)methyl)-4,5,6,7-tetrahydro-1H-pyrazolo[3,4-c]pyridine-3- |
| | carboxamide |
| 1107 | N-benzyl-1-(3-chlorophenyl)-6-(2-(2-hydroxyethyl)-1,2,3,4-tetrahydroisoquinolin-7-yl)- |
| | 7-oxo-4,5,6,7-tetrahydro-1H-pyrazolo[3,4-c]pyridine-3-carboxamide |
| 1108 | (R)-1-(3-chlorophenyl)-6-(2-(2-hydroxyethyl)-1,2,3,4-tetrahydroisoquinolin-7-yl)-7- |
| | oxo-N-(tetrahydrofuran-3-yl)-4,5,6,7-tetrahydro-1H-pyrazolo[3,4-c]pyridine-3- |
| | carboxamide |
| 1109 | 1-(3-chlorophenyl)-N-(furan-2-ylmethyl)-6-(2-(2-hydroxyethyl)-1,2,3,4- |
| | tetrahydroisoquinolin-7-yl)-7-oxo-4,5,6,7-tetrahydro-1H-pyrazolo[3,4-c]pyridine-3- |
| | carboxamide |
| 1110 | 1-(3-chlorophenyl)-6-(2-(2-hydroxyethyl)-1,2,3,4-tetrahydroisoquinolin-7-yl)-N-((4- |
| | methylpyridin-3-yl)methyl)-7-oxo-4,5,6,7-tetrahydro-1H-pyrazolo[3,4-c]pyridine-3- |
| | carboxamide |
| 1111 | 1-(3-chlorophenyl)-6-(2-(2-hydroxyethyl)-1,2,3,4-tetrahydroisoquinolin-7-yl)-N-((2- |
| | methylpyridin-3-yl)methyl)-7-oxo-4,5,6,7-tetrahydro-1H-pyrazolo[3,4-c]pyridine-3- |
| | carboxamide |
| 1112 | 1-(3-chlorophenyl)-N-(2,2-dimethyltetrahydrofuran-3-yl)-6-(2-(2-hydroxyethyl)- |
| | 1,2,3,4-tetrahydroisoquinolin-7-yl)-7-oxo-4,5,6,7-tetrahydro-1H-pyrazolo[3,4- |
| | c]pyridine-3-carboxamide |
| 1113 | (S)-1-(3-chlorophenyl)-N-(cyclopropylmethyl)-6-(2-(1-hydroxypropan-2-yl)-1,2,3,4- |
| | tetrahydroisoquinolin-7-yl)-7-oxo-4,5,6,7-tetrahydro-1H-pyrazolo[3,4-c]pyridine-3- |
| | carboxamide |
| 1114 | 1-(3-chlorophenyl)-N-(cyclopropylmethyl)-6-(2-(2-hydroxyacetyl)-1,2,3,4- |
| | tetrahydroisoquinolin-7-yl)-7-oxo-4,5,6,7-tetrahydro-1H-pyrazolo[3,4-c]pyridine-3- |
| | carboxamide |
| 1115 | 1-(3-chlorophenyl)-N-(cyclopropylmethyl)-7-oxo-6-(2-(tetrahydrofuran-3-yl)-1,2,3,4- |
| | tetrahydroisoquinolin-7-yl)-4,5,6,7-tetrahydro-1H-pyrazolo[3,4-c]pyridine-3- |
| | carboxamide |
| 1116 | 1-(3-chlorophenyl)-N-(cyclopropylmethyl)-7-oxo-6-(2-((tetrahydrofuran-3-yl)methyl)- |
| | 1,2,3,4-tetrahydroisoquinolin-7-yl)-4,5,6,7-tetrahydro-1H-pyrazolo[3,4-c]pyridine-3- |
| | carboxamide |
| 1117 | 1-(3-chlorophenyl)-N-(cyclopropylmethyl)-6-(6-ethyl-5,6,7,8-tetrahydro-2,6- |
| | naphthyridin-3-yl)-7-oxo-4,5,6,7-tetrahydro-1H-pyrazolo[3,4-c]pyridine-3- |
| | carboxamide |
| 1118 | 1-(3-chlorophenyl)-N-(cyclopropylmethyl)-6-(6-(2-hydroxyethyl)-5,6,7,8-tetrahydro- |
| | 2,6-naphthyridin-3-yl)-7-oxo-4,5,6,7-tetrahydro-1H-pyrazolo[3,4-c]pyridine-3- |
| | carboxamide |
| 1119 | 1-(3-chlorophenyl)-N-(cyclopropylmethyl)-6-(2-(2-hydroxycyclopentyl)-1,2,3,4- |
| | tetrahydroisoquinolin-7-yl)-7-oxo-4,5,6,7-tetrahydro-1H-pyrazolo[3,4-c]pyridine-3- |
| | carboxamide |
| 1120 | (R)-1-(3-chlorophenyl)-N-(cyclopropylmethyl)-7-oxo-6-(2-((5-oxopyrrolidin-2- |
| | yl)methyl)-1,2,3,4-tetrahydroisoquinolin-7-yl)-4,5,6,7-tetrahydro-1H-pyrazolo[3,4- |
| | c]pyridine-3-carboxamide |
| 1121 | 1-(3-chlorophenyl)-N-(cyclopropylmethyl)-6-(2-ethyl-5-fluoro-1,2,3,4- |
| | tetrahydroisoquinolin-7-yl)-7-oxo-4,5,6,7-tetrahydro-1H-pyrazolo[3,4-c]pyridine-3- |
| | carboxamide |
| 1122 | 1-(3-chlorophenyl)-N-(cyclopropylmethyl)-6-(5-fluoro-2-(2-hydroxyethyl)-1,2,3,4- |
| | tetrahydroisoquinolin-7-yl)-7-oxo-4,5,6,7-tetrahydro-1H-pyrazolo[3,4-c]pyridine-3- |
| | carboxamide |
| 1123 | 1-(3-chlorophenyl)-N-(cyclopropylmethyl)-6-(6-fluoro-2-(2-hydroxyethyl)-1,2,3,4- |
| | tetrahydroisoquinolin-7-yl)-7-oxo-4,5,6,7-tetrahydro-1H-pyrazolo[3,4-c]pyridine-3- |
| | carboxamide |
| 1124 | (S)-1-(3-chlorophenyl)-N-(cyclopropylmethyl)-7-oxo-6-(2-((5-oxopyrrolidin-2- |
| | yl)methyl)-1,2,3,4-tetrahydroisoquinolin-7-yl)-4,5,6,7-tetrahydro-1H-pyrazolo[3,4- |
| | c]pyridine-3-carboxamide |
| 1125 | 1-(3-chlorophenyl)-6-(6-fluoro-2-(2-hydroxyethyl)-1,2,3,4-tetrahydroisoquinolin-7-yl)- |
| | N-((3-methyl-1H-pyrazol-5-yl)methyl)-7-oxo-4,5,6,7-tetrahydro-1H-pyrazolo[3,4- |
| | c]pyridine-3-carboxamide |
| 1126 | 1-(3-chlorophenyl)-6-(2-(2-hydroxyethyl)-1,2,3,4-tetrahydroisoquinolin-7-yl)-N-((5- |
| | methylpyridin-3-yl)methyl)-7-oxo-4,5,6,7-tetrahydro-1H-pyrazolo[3,4-c]pyridine-3- |
| | carboxamide |
| 1127 | 1-(3-chlorophenyl)-N-((3-cyclopropyl-1H-pyrazol-5-yl)methyl)-6-(2-(2-hydroxyethyl)- |
| | 1,2,3,4-tetrahydroisoquinolin-7-yl)-7-oxo-4,5,6,7-tetrahydro-1H-pyrazolo[3,4- |
| | c]pyridine-3-carboxamide |
| 1128 | 1-(3-chlorophenyl)-N-((4,6-dimethylpyridin-3-yl)methyl)-6-(2-(2-hydroxyethyl)-1,2,3,4- |
| | tetrahydroisoquinolin-7-yl)-7-oxo-4,5,6,7-tetrahydro-1H-pyrazolo[3,4-c]pyridine-3- |
| | carboxamide |
| 1129 | 1-(3-chlorophenyl)-6-(2-methyl-1,2,3,4-tetrahydroisoquinolin-7-yl)-N-((4- |
| | methylpyridin-3-yl)methyl)-7-oxo-4,5,6,7-tetrahydro-1H-pyrazolo[3,4-c]pyridine-3- |
| | carboxamide |
| 1130 | 1-(3-chlorophenyl)-N-((3-fluoropyridin-2-yl)methyl)-6-(2-(2-hydroxyethyl)-1,2,3,4- |
| | tetrahydroisoquinolin-7-yl)-7-oxo-4,5,6,7-tetrahydro-1H-pyrazolo[3,4-c]pyridine-3- |
| | carboxamide |
| 1131 | (S)-1-(3-chlorophenyl)-6-(2-(2-hydroxyethyl)-1,2,3,4-tetrahydroisoquinolin-7-yl)-7- |
| | oxo-N-(1-(pyridin-3-yl)ethyl)-4,5,6,7-tetrahydro-1H-pyrazolo[3,4-c]pyridine-3- |
| | carboxamide |
| 1132 | 1-(3-chlorophenyl)-6-(2-(2-hydroxyethyl)-1,2,3,4-tetrahydroisoquinolin-7-yl)-N-((1- |
| | methyl-1H-pyrazol-3-yl)methyl)-7-oxo-4,5,6,7-tetrahydro-1H-pyrazolo[3,4-c]pyridine- |
| | 3-carboxamide |
| 1133 | 1-(3-chlorophenyl)-6-(2-(2-hydroxyethyl)-1,2,3,4-tetrahydroisoquinolin-7-yl)-N-((1- |
| | methyl-1H-pyrazol-4-yl)methyl)-7-oxo-4,5,6,7-tetrahydro-1H-pyrazolo[3,4-c]pyridine- |
| | 3-carboxamide |
| 1134 | 1-(3-chlorophenyl)-6-(6-fluoro-2-(2-hydroxyethyl)-1,2,3,4-tetrahydroisoquinolin-7-yl)- |
| | N-((4-methylpyridin-3-yl)methyl)-7-oxo-4,5,6,7-tetrahydro-1H-pyrazolo[3,4- |
| | c]pyridine-3-carboxamide |
| 1135 | 1-(3-chlorophenyl)-N-((6-methoxypyridin-3-yl)methyl)-6-(2-methyl-1,2,3,4- |
| | tetrahydroisoquinolin-7-yl)-7-oxo-4,5,6,7-tetrahydro-1H-pyrazolo[3,4-c]pyridine-3- |
| | carboxamide |
| 1136 | 1-(3-chlorophenyl)-6-(2-(2-hydroxyethyl)-1,2,3,4-tetrahydroisoquinolin-7-yl)-N-((3- |
| | methylpyridin-2-yl)methyl)-7-oxo-4,5,6,7-tetrahydro-1H-pyrazolo[3,4-c]pyridine-3- |
| | carboxamide |
| 1137 | 1-(3-chlorophenyl)-N-((5-fluoropyridin-3-yl)methyl)-6-(2-(2-hydroxyethyl)-1,2,3,4- |
| | tetrahydroisoquinolin-7-yl)-7-oxo-4,5,6,7-tetrahydro-1H-pyrazolo[3,4-c]pyridine-3- |
| | carboxamide |
| 1138 | 1-(3-chlorophenyl)-6-(2-(2-hydroxyethyl)-1,2,3,4-tetrahydroisoquinolin-7-yl)-N-((6- |
| | methoxypyridin-3-yl)methyl)-7-oxo-4,5,6,7-tetrahydro-1H-pyrazolo[3,4-c]pyridine-3- |
| | carboxamide |
| 1139 | 1-(3-chlorophenyl)-6-(2-(2-hydroxyethyl)-1,2,3,4-tetrahydroisoquinolin-7-yl)-N-((6- |
| | methylpyridin-2-yl)methyl)-7-oxo-4,5,6,7-tetrahydro-1H-pyrazolo[3,4-c]pyridine-3- |
| | carboxamide |
| 1140 | 1-(3-chlorophenyl)-6-(2-ethyl-1,2,3,4-tetrahydroisoquinolin-7-yl)-N-((4-methylpyridin- |
| | 3-yl)methyl)-7-oxo-4,5,6,7-tetrahydro-1H-pyrazolo[3,4-c]pyridine-3-carboxamide |
| 1141 | 1-(3-chlorophenyl)-6-(2-(2-hydroxyethyl)-1,2,3,4-tetrahydroisoquinolin-7-yl)-N-((5- |
| | methoxypyridin-3-yl)methyl)-7-oxo-4,5,6,7-tetrahydro-1H-pyrazolo[3,4-c]pyridine-3- |
| | carboxamide |
| 1142 | 1-(3-chlorophenyl)-N-((2,2-difluorocyclopropyl)methyl)-6-(2-(2-hydroxyethyl)-1,2,3,4- |
| | tetrahydroisoquinolin-7-yl)-7-oxo-4,5,6,7-tetrahydro-1H-pyrazolo[3,4-c]pyridine-3- |
| | carboxamide |
| 1143 | 1-(3-chlorophenyl)-N-(cyclopropylmethyl)-6-(2′-(2-hydroxyethyl)-2′,3′-dihydro-1′H- |
| | spiro[cyclopropane-1,4′-isoquinolin]-7′-yl)-7-oxo-4,5,6,7-tetrahydro-1H-pyrazolo[3,4- |
| | c]pyridine-3-carboxamide |
| 1144 | 1-(3-chlorophenyl)-6-(2′-(2-hydroxyethyl)-2′,3′-dihydro-1′H-spiro[cyclopropane-1,4′- |
| | isoquinolin]-7′-yl)-N-((4-methylpyridin-3-yl)methyl)-7-oxo-4,5,6,7-tetrahydro-1H- |
| | pyrazolo[3,4-c]pyridine-3-carboxamide |
| 1145 | (R)-1-(3-chlorophenyl)-6-(2-(2-hydroxyethyl)-1,2,3,4-tetrahydroisoquinolin-7-yl)-7- |
| | oxo-N-(1-(pyridin-3-yl)ethyl)-4,5,6,7-tetrahydro-1H-pyrazolo[3,4-c]pyridine-3- |
| | carboxamide |
| 1146 | 1-(3-chlorophenyl)-N-((1-cyclopropyl-1H-pyrazol-3-yl)methyl)-6-(2-(2-hydroxyethyl)- |
| | 1,2,3,4-tetrahydroisoquinolin-7-yl)-7-oxo-4,5,6,7-tetrahydro-1H-pyrazolo[3,4- |
| | c]pyridine-3-carboxamide |
| 1147 | 1-(3-chlorophenyl)-6-(2-(2-hydroxyethyl)-1,2,3,4-tetrahydroisoquinolin-7-yl)-N-((6- |
| | methylpyridin-3-yl)methyl)-7-oxo-4,5,6,7-tetrahydro-1H-pyrazolo[3,4-c]pyridine-3- |
| | carboxamide |
| 1148 | (R)-1-(3-chlorophenyl)-6-(2-(2-hydroxyethyl)-4-methyl-1,2,3,4-tetrahydroisoquinolin- |
| | 7-yl)-N-((4-methylpyridin-3-yl)methyl)-7-oxo-4,5,6,7-tetrahydro-1H-pyrazolo[3,4- |
| | c]pyridine-3-carboxamide |
| 1149 | 1-(3-chlorophenyl)-N-(cyclopropylmethyl)-6-(2-methyl-1-oxo-1,2,3,4- |
| | tetrahydroisoquinolin-7-yl)-7-oxo-4,5,6,7-tetrahydro-1H-pyrazolo[3,4-c]pyridine-3- |
| | carboxamide |
| 1150 | 1-(3-chlorophenyl)-N-((3-cyclopropyl-1H-pyrazol-5-yl)methyl)-6-(2-ethyl-1,2,3,4- |
| | tetrahydroisoquinolin-7-yl)-7-oxo-4,5,6,7-tetrahydro-1H-pyrazolo[3,4-c]pyridine-3- |
| | carboxamide |
| 1151 | 1-(3-chlorophenyl)-6-(2-(2-hydroxyethyl)-1,2,3,4-tetrahydroisoquinolin-7-yl)-N- |
| | (oxazol-2-ylmethyl)-7-oxo-4,5,6,7-tetrahydro-1H-pyrazolo[3,4-c]pyridine-3- |
| | carboxamide |
| 1152 | (R)-1-(3-chlorophenyl)-N-(cyclopropylmethyl)-6-(2-(2-hydroxyethyl)-4-methyl-1,2,3,4- |
| | tetrahydroisoquinolin-7-yl)-7-oxo-4,5,6,7-tetrahydro-1H-pyrazolo[3,4-c]pyridine-3- |
| | carboxamide |
| 1153 | (R)-1-(3-chlorophenyl)-N-(cyclopropylmethyl)-6-(2-ethyl-4-methyl-1,2,3,4- |
| | tetrahydroisoquinolin-7-yl)-7-oxo-4,5,6,7-tetrahydro-1H-pyrazolo[3,4-c]pyridine-3- |
| | carboxamide |
| 1154 | 1-(3-chlorophenyl)-N-((4-chloropyridin-3-yl)methyl)-6-(2-(2-hydroxyethyl)-1,2,3,4- |
| | tetrahydroisoquinolin-7-yl)-7-oxo-4,5,6,7-tetrahydro-1H-pyrazolo[3,4-c]pyridine-3- |
| | carboxamide |
| 1155 | N-((4-chloro-1-methyl-1H-pyrazol-3-yl)methyl)-1-(3-chlorophenyl)-6-(2-(2- |
| | hydroxyethyl)-1,2,3,4-tetrahydroisoquinolin-7-yl)-7-oxo-4,5,6,7-tetrahydro-1H- |
| | pyrazolo[3,4-c]pyridine-3-carboxamide |
| 1156 | 1-(3-chlorophenyl)-6-(2-(2-hydroxyethyl)-1,2,3,4-tetrahydroisoquinolin-7-yl)-N-((3- |
| | (methoxymethyl)-1H-pyrazol-5-yl)methyl)-7-oxo-4,5,6,7-tetrahydro-1H-pyrazolo[3,4- |
| | c]pyridine-3-carboxamide |
| 1157 | 1-(3-chlorophenyl)-N-((1,4-dimethyl-1H-pyrazol-3-yl)methyl)-6-(2-(2-hydroxyethyl)- |
| | 1,2,3,4-tetrahydroisoquinolin-7-yl)-7-oxo-4,5,6,7-tetrahydro-1H-pyrazolo[3,4- |
| | c]pyridine-3-carboxamide |
| 1158 | (R)-1-(3-chlorophenyl)-6-(2-ethyl-4-methyl-1,2,3,4-tetrahydroisoquinolin-7-yl)-N-((4- |
| | methylpyridin-3-yl)methyl)-7-oxo-4,5,6,7-tetrahydro-1H-pyrazolo[3,4-c]pyridine-3- |
| | carboxamide |
| 1159 | 1-(3-chlorophenyl)-6-(2-methyl-1-oxo-1,2,3,4-tetrahydroisoquinolin-7-yl)-N-((4- |
| | methylpyridin-3-yl)methyl)-7-oxo-4,5,6,7-tetrahydro-1H-pyrazolo[3,4-c]pyridine-3- |
| | carboxamide |
| 1160 | 1-(3-chlorophenyl)-6-(2-(2-hydroxyethyl)-1,2,3,4-tetrahydroisoquinolin-7-yl)-N-((3- |
| | methoxypyridin-2-yl)methyl)-7-oxo-4,5,6,7-tetrahydro-1H-pyrazolo[3,4-c]pyridine-3- |
| | carboxamide |
| 1161 | 1-(3-chlorophenyl)-N-((3-fluoropyridin-4-yl)methyl)-6-(2-(2-hydroxyethyl)-1,2,3,4- |
| | tetrahydroisoquinolin-7-yl)-7-oxo-4,5,6,7-tetrahydro-1H-pyrazolo[3,4-c]pyridine-3- |
| | carboxamide |
| 1162 | 1-(3-chlorophenyl)-6-(2-(2-hydroxyethyl)-1,2,3,4-tetrahydroisoquinolin-7-yl)-7-oxo-N- |
| | ((4-(trifluoromethyl)pyridin-3-yl)methyl)-4,5,6,7-tetrahydro-1H-pyrazolo[3,4- |
| | c]pyridine-3-carboxamide |
| 1163 | 1-(3-chlorophenyl)-6-(2-(2-hydroxyethyl)-1,2,3,4-tetrahydroisoquinolin-7-yl)-N-((3- |
| | methylpyridin-4-yl)methyl)-7-oxo-4,5,6,7-tetrahydro-1H-pyrazolo[3,4-c]pyridine-3- |
| | carboxamide |
| 1164 | 1-(3-chlorophenyl)-N-(2,2-dimethyltetrahydrofuran-3-yl)-7-oxo-6-(3-(piperidin-1- |
| | ylmethyl)phenyl)-4,5,6,7-tetrahydro-1H-pyrazolo[3,4-c]pyridine-3-carboxamide |
| 1165 | 1-(3-chlorophenyl)-N-(4-hydroxybicyclo[1.1.1]pentan-2-yl)-6-(2-methyl-1,2,3,4- |
| | tetrahydroisoquinolin-7-yl)-7-oxo-4,5,6,7-tetrahydro-1H-pyrazolo[3,4-c]pyridine-3- |
| | carboxamide |
| 1166 | 1-(3-chlorophenyl)-N-((1,5-dimethyl-1H-pyrazol-3-yl)methyl)-6-(2-(2-hydroxyethyl)- |
| | 1,2,3,4-tetrahydroisoquinolin-7-yl)-7-oxo-4,5,6,7-tetrahydro-1H-pyrazolo[3,4- |
| | c]pyridine-3-carboxamide |
| 1167 | 1-(3-chlorophenyl)-6-(2-(2-hydroxyethyl)-1,2,3,4-tetrahydroisoquinolin-7-yl)-N- |
| | (isoxazol-4-ylmethyl)-7-oxo-4,5,6,7-tetrahydro-1H-pyrazolo[3,4-c]pyridine-3- |
| | carboxamide |
| 1168 | 1-(3-chlorophenyl)-N-((3-methyl-1H-pyrazol-5-yl)methyl)-7-oxo-6-(3-(piperidin-1- |
| | ylmethyl)phenyl)-4,5,6,7-tetrahydro-1H-pyrazolo[3,4-c]pyridine-3-carboxamide |
| 1169 | 1-(3-chlorophenyl)-N-(cyclopropylmethyl)-6-(2-ethyl-5-(methoxymethyl)-1,2,3,4- |
| | tetrahydroisoquinolin-7-yl)-7-oxo-4,5,6,7-tetrahydro-1H-pyrazolo[3,4-c]pyridine-3- |
| | carboxamide |
| 1170 | 1-(3-chlorophenyl)-N-((3-chloropyridin-2-yl)methyl)-6-(2-(2-hydroxyethyl)-1,2,3,4- |
| | tetrahydroisoquinolin-7-yl)-7-oxo-4,5,6,7-tetrahydro-1H-pyrazolo[3,4-c]pyridine-3- |
| | carboxamide |
| 1171 | 1-(3-chlorophenyl)-6-(2-(2-hydroxyethyl)-1,2,3,4-tetrahydroisoquinolin-7-yl)-N-((2- |
| | methylpyridin-4-yl)methyl)-7-oxo-4,5,6,7-tetrahydro-1H-pyrazolo[3,4-c]pyridine-3- |
| | carboxamide |
| 1172 | 1-(3-chlorophenyl)-6-(2-(2-hydroxyethyl)-1,2,3,4-tetrahydroisoquinolin-7-yl)-N-((3- |
| | methoxypyridin-4-yl)methyl)-7-oxo-4,5,6,7-tetrahydro-1H-pyrazolo[3,4-c]pyridine-3- |
| | carboxamide |
| 1173 | 1-(3-chlorophenyl)-N-((6-methoxypyridin-3-yl)methyl)-7-oxo-6-(3-(piperidin-1- |
| | ylmethyl)phenyl)-4,5,6,7-tetrahydro-1H-pyrazolo[3,4-c]pyridine-3-carboxamide |
| 1174 | 1-(3-chlorophenyl)-N-((4-chloropyridin-3-yl)methyl)-7-oxo-6-(3-(piperidin-1- |
| | ylmethyl)phenyl)-4,5,6,7-tetrahydro-1H-pyrazolo[3,4-c]pyridine-3-carboxamide |
| 1175 | 1-(3-chlorophenyl)-N-(cyclopropylmethyl)-6-(2-(2-fluoroethyl)-1,2,3,4- |
| | tetrahydroisoquinolin-7-yl)-7-oxo-4,5,6,7-tetrahydro-1H-pyrazolo[3,4-c]pyridine-3- |
| | carboxamide |
| 1176 | 1-(3-chlorophenyl)-N-(cyclopropylmethyl)-6-(2-(2-(methylsulfonyl)ethyl)-1,2,3,4- |
| | tetrahydroisoquinolin-7-yl)-7-oxo-4,5,6,7-tetrahydro-1H-pyrazolo[3,4-c]pyridine-3- |
| | carboxamide |
| 1177 | 1-(3-chlorophenyl)-N-((4-methylpyridin-3-yl)methyl)-7-oxo-6-(3-(piperidin-1- |
| | ylmethyl)phenyl)-4,5,6,7-tetrahydro-1H-pyrazolo[3,4-c]pyridine-3-carboxamide |
| 1178 | 1-(3-chlorophenyl)-N-((1,4-dimethyl-1H-pyrazol-3-yl)methyl)-7-oxo-6-(3-(piperidin-1- |
| | ylmethyl)phenyl)-4,5,6,7-tetrahydro-1H-pyrazolo[3,4-c]pyridine-3-carboxamide |
| 1179 | 1-(3-chlorophenyl)-N-(4-hydroxytetrahydrofuran-3-yl)-6-(2-methyl-1-oxo-1,2,3,4- |
| | tetrahydroisoquinolin-7-yl)-7-oxo-4,5,6,7-tetrahydro-1H-pyrazolo[3,4-c]pyridine-3- |
| | carboxamide |
| 1180 | 1-(3-chlorophenyl)-N-(2-hydroxy-1-(tetrahydrofuran-3-yl)ethyl)-6-(2-methyl-1-oxo- |
| | 1,2,3,4-tetrahydroisoquinolin-7-yl)-7-oxo-4,5,6,7-tetrahydro-1H-pyrazolo[3,4- |
| | c]pyridine-3-carboxamide |
| 1181 | 1-(3-chlorophenyl)-N-((5-methyl-1,3,4-thiadiazol-2-yl)methyl)-6-(2-methyl-1-oxo- |
| | 1,2,3,4-tetrahydroisoquinolin-7-yl)-7-oxo-4,5,6,7-tetrahydro-1H-pyrazolo[3,4- |
| | c]pyridine-3-carboxamide |
| 1182 | 1-(3-chlorophenyl)-6-(2-methyl-1-oxo-1,2,3,4-tetrahydroisoquinolin-7-yl)-7-oxo- |
| | 4,5,6,7-tetrahydro-1H-pyrazolo[3,4-c]pyridine-3-carboxylic acid |
| 1183 | 7-(1-(3-chlorophenyl)-3-(hydroxymethyl)-7-oxo-1,4,5,7-tetrahydro-6H-pyrazolo[3,4- |
| | c]pyridin-6-yl)-2-methyl-3,4-dihydroisoquinolin-1(2H)-one |
| 1184 | 7-(1-(3-chlorophenyl)-3-(methoxymethyl)-7-oxo-1,4,5,7-tetrahydro-6H-pyrazolo[3,4- |
| | c]pyridin-6-yl)-2-methyl-3,4-dihydroisoquinolin-1(2H)-one |
| 1185 | 1-(3-chlorophenyl)-N-((3,5-dimethylisoxazol-4-yl)methyl)-6-(2-methyl-1-oxo-1,2,3,4- |
| | tetrahydroisoquinolin-7-yl)-7-oxo-4,5,6,7-tetrahydro-1H-pyrazolo[3,4-c]pyridine-3- |
| | carboxamide |
| 1186 | 1-(3-chlorophenyl)-N-((5-methoxy-1-methyl-1H-pyrazol-3-yl)methyl)-6-(2-methyl-1- |
| | oxo-1,2,3,4-tetrahydroisoquinolin-7-yl)-7-oxo-4,5,6,7-tetrahydro-1H-pyrazolo[3,4- |
| | c]pyridine-3-carboxamide |
| 1187 | 1-(3-chlorophenyl)-N-(2-hydroxy-1-(pyridin-3-yl)ethyl)-6-(2-methyl-1-oxo-1,2,3,4- |
| | tetrahydroisoquinolin-7-yl)-7-oxo-4,5,6,7-tetrahydro-1H-pyrazolo[3,4-c]pyridine-3- |
| | carboxamide |
| 1188 | 6-(1-acetylindolin-6-yl)-1-(3-chlorophenyl)-N-((4-methylpyridin-3-yl)methyl)-7-oxo- |
| | 4,5,6,7-tetrahydro-1H-pyrazolo[3,4-c]pyridine-3-carboxamide |
| 1189 | 1-(3-chlorophenyl)-N-((4,6-dimethylpyridin-3-yl)methyl)-6-(2-methyl-1-oxo-1,2,3,4- |
| | tetrahydroisoquinolin-7-yl)-7-oxo-4,5,6,7-tetrahydro-1H-pyrazolo[3,4-c]pyridine-3- |
| | carboxamide |
| 1190 | 1-(3-chlorophenyl)-N-(cyclopropylmethyl)-6-(2-methyl-1-oxo-1,2,3,4- |
| | tetrahydroisoquinolin-6-yl)-7-oxo-4,5,6,7-tetrahydro-1H-pyrazolo[3,4-c]pyridine-3- |
| | carboxamide |
| 1191 | 1-(3-chlorophenyl)-N-(cyclopropylmethyl)-6-(2-(2-hydroxyethyl)-4-methoxy-1,2,3,4- |
| | tetrahydroisoquinolin-7-yl)-7-oxo-4,5,6,7-tetrahydro-1H-pyrazolo[3,4-c]pyridine-3- |
| | carboxamide |
| 1192 | 1-(3-chlorophenyl)-N-(cyclopropylmethyl)-6-(2-methyl-3-oxoisoindolin-5-yl)-7-oxo- |
| | 4,5,6,7-tetrahydro-1H-pyrazolo[3,4-c]pyridine-3-carboxamide |
| 1193 | 1-(3-chlorophenyl)-6-(2-methyl-1-oxo-1,2,3,4-tetrahydroisoquinolin-7-yl)-N-((2- |
| | methylthiazol-5-yl)methyl)-7-oxo-4,5,6,7-tetrahydro-1H-pyrazolo[3,4-c]pyridine-3- |
| | carboxamide |
| 1194 | 1-(3-chlorophenyl)-6-(2-methyl-1-oxo-1,2,3,4-tetrahydroisoquinolin-7-yl)-N-((2- |
| | methyloxazol-5-yl)methyl)-7-oxo-4,5,6,7-tetrahydro-1H-pyrazolo[3,4-c]pyridine-3- |
| | carboxamide |
| 1195 | 1-(3-chlorophenyl)-N-(cyclopropylmethyl)-6-(3-(dimethylcarbamoyl)phenyl)-7-oxo- |
| | 4,5,6,7-tetrahydro-1H-pyrazolo[3,4-c]pyridine-3-carboxamide |
| 1196 | ethyl 1-(3-chlorophenyl)-6-(3-(dimethylcarbamoyl)phenyl)-7-oxo-4,5,6,7-tetrahydro- |
| | 1H-pyrazolo[3,4-c]pyridine-3-carboxylate |
| 1197 | 1-(3-chlorophenyl)-N-(2,2-dimethyltetrahydrofuran-3-yl)-6-(2-methyl-1-oxo-1,2,3,4- |
| | tetrahydroisoquinolin-7-yl)-7-oxo-4,5,6,7-tetrahydro-1H-pyrazolo[3,4-c]pyridine-3- |
| | carboxamide |
| 1198 | 1-(3-chlorophenyl)-6-(2-methyl-1-oxo-1,2,3,4-tetrahydroisoquinolin-7-yl)-7-oxo-N- |
| | ((tetrahydrofuran-3-yl)methyl)-4,5,6,7-tetrahydro-1H-pyrazolo[3,4-c]pyridine-3- |
| | carboxamide |
| 1199 | 1-(3-chlorophenyl)-N-((1,5-dimethyl-1H-pyrazol-3-yl)methyl)-6-(2-methyl-1-oxo- |
| | 1,2,3,4-tetrahydroisoquinolin-7-yl)-7-oxo-4,5,6,7-tetrahydro-1H-pyrazolo[3,4- |
| | c]pyridine-3-carboxamide |
| 1200 | 1-(3-chlorophenyl)-6-(2-methyl-1-oxo-1,2,3,4-tetrahydroisoquinolin-7-yl)-N-((5- |
| | methyl-1H-pyrazol-3-yl)methyl)-7-oxo-4,5,6,7-tetrahydro-1H-pyrazolo[3,4-c]pyridine- |
| | 3-carboxamide |
| 1201 | 1-(3-chlorophenyl)-6-(2-methyl-1-oxo-1,2,3,4-tetrahydroisoquinolin-7-yl)-N-((5- |
| | methylpyridin-3-yl)methyl)-7-oxo-4,5,6,7-tetrahydro-1H-pyrazolo[3,4-c]pyridine-3- |
| | carboxamide |
| 1202 | 1-(3-chlorophenyl)-6-(2-methyl-1-oxo-1,2,3,4-tetrahydroisoquinolin-7-yl)-7-oxo-N- |
| | (pyridazin-3-ylmethyl)-4,5,6,7-tetrahydro-1H-pyrazolo[3,4-c]pyridine-3-carboxamide |
| 1203 | 1-(3-chlorophenyl)-6-(2-methyl-1-oxo-1,2,3,4-tetrahydroisoquinolin-7-yl)-7-oxo-N- |
| | (tetrahydro-2H-pyran-4-yl)-4,5,6,7-tetrahydro-1H-pyrazolo[3,4-c]pyridine-3- |
| | carboxamide |
| 1204 | 1-(3-chlorophenyl)-6-(2-(2-hydroxyethyl)-1,2,3,4-tetrahydroisoquinolin-7-yl)-7-oxo-N- |
| | (pyridazin-3-ylmethyl)-4,5,6,7-tetrahydro-1H-pyrazolo[3,4-c]pyridine-3-carboxamide |
| 1205 | 1-(3-chlorophenyl)-N-((1,5-dimethyl-1H-pyrazol-3-yl)methyl)-6-(2-methyl-1-oxo- |
| | 1,2,3,4-tetrahydroisoquinolin-6-yl)-7-oxo-4,5,6,7-tetrahydro-1H-pyrazolo[3,4- |
| | c]pyridine-3-carboxamide |
| 1206 | 1-(3-chlorophenyl)-N-((1,5-dimethyl-1H-pyrazol-3-yl)methyl)-6-(2-methyl-3- |
| | oxoisoindolin-5-yl)-7-oxo-4,5,6,7-tetrahydro-1H-pyrazolo[3,4-c]pyridine-3- |
| | carboxamide |
| 1207 | 1-(3-chlorophenyl)-N-(cyclopropylmethyl)-6-(1-methyl-2-oxo-1,2,3,4- |
| | tetrahydroquinolin-7-yl)-7-oxo-4,5,6,7-tetrahydro-1H-pyrazolo[3,4-c]pyridine-3- |
| | carboxamide |
| 1208 | 1-(3-chlorophenyl)-N-((1,5-dimethyl-1H-pyrazol-3-yl)methyl)-6-(1-methyl-2-oxo- |
| | 1,2,3,4-tetrahydroquinolin-7-yl)-7-oxo-4,5,6,7-tetrahydro-1H-pyrazolo[3,4-c]pyridine- |
| | 3-carboxamide |
| 1209 | 1-(3-chlorophenyl)-N-((4-methylpyridin-3-yl)methyl)-7-oxo-6-(1-oxo-2-propyl-1,2,3,4- |
| | tetrahydroisoquinolin-7-yl)-4,5,6,7-tetrahydro-1H-pyrazolo[3,4-c]pyridine-3- |
| | carboxamide |
| 1210 | 1-(3-chlorophenyl)-N-((1,5-dimethyl-1H-pyrazol-3-yl)methyl)-7-oxo-6-(1-oxo-2-propyl- |
| | 1,2,3,4-tetrahydroisoquinolin-7-yl)-4,5,6,7-tetrahydro-1H-pyrazolo[3,4-c]pyridine-3- |
| | carboxamide |
| 1211 | 1-(3-chlorophenyl)-N-(cyclopropylmethyl)-7-oxo-6-(1-oxo-2-propyl-1,2,3,4- |
| | tetrahydroisoquinolin-7-yl)-4,5,6,7-tetrahydro-1H-pyrazolo[3,4-c]pyridine-3- |
| | carboxamide |
| 1212 | 1-(3-chlorophenyl)-6-(2-methyl-1-oxo-1,2-dihydroisoquinolin-7-yl)-N-((4- |
| | methylpyridin-3-yl)methyl)-7-oxo-4,5,6,7-tetrahydro-1H-pyrazolo[3,4-c]pyridine-3- |
| | carboxamide |
| 1213 | 1-(3-chlorophenyl)-N-((5-methoxypyridin-3-yl)methyl)-6-(2-methyl-1-oxo-1,2,3,4- |
| | tetrahydroisoquinolin-7-yl)-7-oxo-4,5,6,7-tetrahydro-1H-pyrazolo[3,4-c]pyridine-3- |
| | carboxamide |
| 1214 | (R)-1-(3-chlorophenyl)-6-(2-methyl-1-oxo-1,2,3,4-tetrahydroisoquinolin-7-yl)-7-oxo-N- |
| | (1-(pyridin-3-yl)ethyl)-4,5,6,7-tetrahydro-1H-pyrazolo[3,4-c]pyridine-3-carboxamide |
| 1215 | (S)-1-(3-chlorophenyl)-6-(2-methyl-1-oxo-1,2,3,4-tetrahydroisoquinolin-7-yl)-7-oxo-N- |
| | (1-(pyridin-3-yl)ethyl)-4,5,6,7-tetrahydro-1H-pyrazolo[3,4-c]pyridine-3-carboxamide |
| 1216 | 1-(3-chlorophenyl)-N-(cyclopropylmethyl)-6-(2-(2-hydroxyethyl)-1-oxo-1,2,3,4- |
| | tetrahydroisoquinolin-7-yl)-7-oxo-4,5,6,7-tetrahydro-1H-pyrazolo[3,4-c]pyridine-3- |
| | carboxamide |
| 1217 | 1-(3-chlorophenyl)-N-((1,4-dimethyl-1H-pyrazol-3-yl)methyl)-6-(2-(2-hydroxyethyl)-1- |
| | oxo-1,2,3,4-tetrahydroisoquinolin-7-yl)-7-oxo-4,5,6,7-tetrahydro-1H-pyrazolo[3,4- |
| | c]pyridine-3-carboxamide |
| 1218 | 1-(3-chlorophenyl)-6-(2-(2-hydroxyethyl)-1,2,3,4-tetrahydroisoquinolin-7-yl)-7-oxo-N- |
| | (pyridazin-4-ylmethyl)-4,5,6,7-tetrahydro-1H-pyrazolo[3,4-c]pyridine-3-carboxamide |
| 1219 | 1-(3-chlorophenyl)-N-((1,5-dimethyl-1H-pyrazol-3-yl)methyl)-6-(2-ethyl-1-oxo-1,2,3,4- |
| | tetrahydroisoquinolin-7-yl)-7-oxo-4,5,6,7-tetrahydro-1H-pyrazolo[3,4-c]pyridine-3- |
| | carboxamide |
| 1220 | 1-(3-chlorophenyl)-N-((1,5-dimethyl-1H-pyrazol-3-yl)methyl)-6-(6-(2-hydroxyethyl)- |
| | 5,6,7,8-tetrahydro-2,6-naphthyridin-3-yl)-7-oxo-4,5,6,7-tetrahydro-1H-pyrazolo[3,4- |
| | c]pyridine-3-carboxamide |
| 1221 | 1-(3-chlorophenyl)-6-(6-(2-hydroxyethyl)-5,6,7,8-tetrahydro-2,6-naphthyridin-3-yl)-N- |
| | ((3-methoxypyridin-2-yl)methyl)-7-oxo-4,5,6,7-tetrahydro-1H-pyrazolo[3,4-c]pyridine- |
| | 3-carboxamide |
| 1222 | 1-(3-chlorophenyl)-N-((1,5-dimethyl-1H-pyrazol-3-yl)methyl)-6-(2-(2-hydroxyethyl)-1- |
| | oxo-1,2,3,4-tetrahydroisoquinolin-7-yl)-7-oxo-4,5,6,7-tetrahydro-1H-pyrazolo[3,4- |
| | c]pyridine-3-carboxamide |
| 1223 | 1-(3-chlorophenyl)-6-(2-(2-hydroxyethyl)-1,2,3,4-tetrahydroisoquinolin-7-yl)-N- |
| | (oxazol-4-ylmethyl)-7-oxo-4,5,6,7-tetrahydro-1H-pyrazolo[3,4-c]pyridine-3- |
| | carboxamide |
| 1224 | 1-(3-chlorophenyl)-N-((1,4-dimethyl-1H-pyrazol-3-yl)methyl)-6-(2-ethyl-1-oxo-1,2,3,4- |
| | tetrahydroisoquinolin-7-yl)-7-oxo-4,5,6,7-tetrahydro-1H-pyrazolo[3,4-c]pyridine-3- |
| | carboxamide |
| 1225 | 1-(3-chlorophenyl)-N-((1,4-dimethyl-1H-pyrazol-3-yl)methyl)-6-(2-methyl-1-oxo- |
| | 1,2,3,4-tetrahydroisoquinolin-7-yl)-7-oxo-4,5,6,7-tetrahydro-1H-pyrazolo[3,4- |
| | c]pyridine-3-carboxamide |
| 1226 | 1-(3-chlorophenyl)-N-(cyclopropylmethyl)-6-(2-(2-hydroxyethyl)-5-methoxy-1,2,3,4- |
| | tetrahydroisoquinolin-7-yl)-7-oxo-4,5,6,7-tetrahydro-1H-pyrazolo[3,4-c]pyridine-3- |
| | carboxamide |
| 1227 | 1-(3-chlorophenyl)-N-((1,5-dimethyl-1H-pyrazol-3-yl)methyl)-6-(6-(2-hydroxyethyl)- |
| | 5,6,7,8-tetrahydropyrido[4,3-c]pyridazin-3-yl)-7-oxo-4,5,6,7-tetrahydro-1H- |
| | pyrazolo[3,4-c]pyridine-3-carboxamide |
| 1229 | 1-(3-chlorophenyl)-6-(6-(2-hydroxyethyl)-5,6,7,8-tetrahydropyrido[4,3-c]pyridazin-3- |
| | yl)-N-((3-methoxypyridin-2-yl)methyl)-7-oxo-4,5,6,7-tetrahydro-1H-pyrazolo[3,4- |
| | c]pyridine-3-carboxamide |
| 1230 | 1-(3-chlorophenyl)-6-(2-(2-hydroxyethyl)-1,2,3,4-tetrahydroisoquinolin-7-yl)-N-((4- |
| | methoxypyridin-2-yl)methyl)-7-oxo-4,5,6,7-tetrahydro-1H-pyrazolo[3,4-c]pyridine-3- |
| | carboxamide |
| 1231 | 1-(3-chlorophenyl)-N-((3-cyclopropyl-1H-pyrazol-5-yl)methyl)-6-(2-methyl-1-oxo- |
| | 1,2,3,4-tetrahydroisoquinolin-7-yl)-7-oxo-4,5,6,7-tetrahydro-1H-pyrazolo[3,4- |
| | c]pyridine-3-carboxamide |
| 1232 | 1-(3-chlorophenyl)-7-oxo-6-(3-(piperidin-1-ylmethyl)phenyl)-4,5,6,7-tetrahydro-1H- |
| | pyrazolo[3,4-c]pyridine-3-carboxylic acid |
| 1233 | 1-(3-chlorophenyl)-N-((3-methoxypyridin-2-yl)methyl)-6-(2-methyl-1-oxo-1,2,3,4- |
| | tetrahydroisoquinolin-7-yl)-7-oxo-4,5,6,7-tetrahydro-1H-pyrazolo[3,4-c]pyridine-3- |
| | carboxamide |
| 1234 | 1-(3-chlorophenyl)-N-((3-cyclopropyl-1H-pyrazol-5-yl)methyl)-6-(2-ethyl-6-fluoro- |
| | 1,2,3,4-tetrahydroisoquinolin-7-yl)-7-oxo-4,5,6,7-tetrahydro-1H-pyrazolo[3,4- |
| | c]pyridine-3-carboxamide |
| 1235 | 1-(3-chlorophenyl)-N-((6-methoxypyridin-3-yl)methyl)-6-(2-methyl-1-oxo-1,2,3,4- |
| | tetrahydroisoquinolin-7-yl)-7-oxo-4,5,6,7-tetrahydro-1H-pyrazolo[3,4-c]pyridine-3- |
| | carboxamide |
| 1236 | 1-(3-chlorophenyl)-6-(2-(2-hydroxyethyl)-1-oxo-1,2,3,4-tetrahydroisoquinolin-7-yl)-N- |
| | ((4-methylpyridin-3-yl)methyl)-7-oxo-4,5,6,7-tetrahydro-1H-pyrazolo[3,4-c]pyridine-3- |
| | carboxamide |
| 1237 | 1-(3-chlorophenyl)-N-((3-cyclopropyl-1H-pyrazol-5-yl)methyl)-7-oxo-6-(3-(piperidin-1- |
| | ylmethyl)phenyl)-4,5,6,7-tetrahydro-1H-pyrazolo[3,4-c]pyridine-3-carboxamide |
| 1238 | 1-(3-chlorophenyl)-N-((1,5-dimethyl-1H-pyrazol-3-yl)methyl)-6-(2-methyl-1-oxo- |
| | 2,3,4,5-tetrahydro-1H-benzo[c]azepin-8-yl)-7-oxo-4,5,6,7-tetrahydro-1H-pyrazolo[3,4- |
| | c]pyridine-3-carboxamide |
| 1239 | 1-(3-chlorophenyl)-N-(cyclopropylmethyl)-6-(2-methyl-1-oxo-2,3,4,5-tetrahydro-1H- |
| | benzo[c]azepin-8-yl)-7-oxo-4,5,6,7-tetrahydro-1H-pyrazolo[3,4-c]pyridine-3- |
| | carboxamide |
| 1240 | 1-(3-chlorophenyl)-N-((4-methylpyridin-3-yl)methyl)-7-oxo-6-(1-oxo-1,2,3,4- |
| | tetrahydroisoquinolin-7-yl)-4,5,6,7-tetrahydro-1H-pyrazolo[3,4-c]pyridine-3- |
| | carboxamide |
| 1241 | 1-(3-chlorophenyl)-N-((1,5-dimethyl-1H-pyrazol-3-yl)methyl)-6-(1-methyl-2-oxo- |
| | 2,3,4,5-tetrahydro-1H-benzo[b]azepin-8-yl)-7-oxo-4,5,6,7-tetrahydro-1H-pyrazolo[3,4- |
| | c]pyridine-3-carboxamide |
| 1242 | 1-(3-chlorophenyl)-N-(cyclopropylmethyl)-6-(1-methyl-2-oxo-2,3,4,5-tetrahydro-1H- |
| | benzo[b]azepin-8-yl)-7-oxo-4,5,6,7-tetrahydro-1H-pyrazolo[3,4-c]pyridine-3- |
| | carboxamide |
| 1243 | 1-(3-chlorophenyl)-N-(cyclopropylmethyl)-6-(3-((3-fluoropyrrolidin-1- |
| | yl)methyl)phenyl)-7-oxo-4,5,6,7-tetrahydro-1H-pyrazolo[3,4-c]pyridine-3-carboxamide |
| 1244 | 1-(3-chlorophenyl)-N-(cyclopropylmethyl)-6-(3-(dimethylcarbamoyl)-4-methylphenyl)- |
| | 7-oxo-4,5,6,7-tetrahydro-1H-pyrazolo[3,4-c]pyridine-3-carboxamide |
| 1245 | N-((1H-pyrazol-3-yl)methyl)-1-(3-chlorophenyl)-6-(2-methyl-1-oxo-1,2,3,4- |
| | tetrahydroisoquinolin-7-yl)-7-oxo-4,5,6,7-tetrahydro-1H-pyrazolo[3,4-c]pyridine-3- |
| | carboxamide |
| 1246 | (S)-N-((1,4-dioxan-2-yl)methyl)-1-(3-chlorophenyl)-6-(2-methyl-1-oxo-1,2,3,4- |
| | tetrahydroisoquinolin-7-yl)-7-oxo-4,5,6,7-tetrahydro-1H-pyrazolo[3,4-c]pyridine-3- |
| | carboxamide |
| 1247 | 1-(3-chlorophenyl)-6-(2-methyl-1-oxo-1,2,3,4-tetrahydroisoquinolin-7-yl)-N-((5- |
| | methyl-1H-1,2,4-triazol-3-yl)methyl)-7-oxo-4,5,6,7-tetrahydro-1H-pyrazolo[3,4- |
| | c]pyridine-3-carboxamide |
| 1248 | 1-(3-chlorophenyl)-6-(2-methyl-1-oxo-1,2,3,4-tetrahydroisoquinolin-7-yl)-N-((4- |
| | methyl-1H-pyrazol-3-yl)methyl)-7-oxo-4,5,6,7-tetrahydro-1H-pyrazolo[3,4-c]pyridine- |
| | 3-carboxamide |
| 1249 | (R)-N-((1,4-dioxan-2-yl)methyl)-1-(3-chlorophenyl)-6-(2-methyl-1-oxo-1,2,3,4- |
| | tetrahydroisoquinolin-7-yl)-7-oxo-4,5,6,7-tetrahydro-1H-pyrazolo[3,4-c]pyridine-3- |
| | carboxamide |
| 1250 | 1-(3-chlorophenyl)-N-((1-(hydroxymethyl)cyclopropyl)methyl)-6-(2-methyl-1-oxo- |
| | 1,2,3,4-tetrahydroisoquinolin-7-yl)-7-oxo-4,5,6,7-tetrahydro-1H-pyrazolo[3,4- |
| | c]pyridine-3-carboxamide |
| 1251 | 1-(3-chlorophenyl)-N-(4-(hydroxymethyl)tetrahydrofuran-3-yl)-6-(2-methyl-1-oxo- |
| | 1,2,3,4-tetrahydroisoquinolin-7-yl)-7-oxo-4,5,6,7-tetrahydro-1H-pyrazolo[3,4- |
| | c]pyridine-3-carboxamide |
| 1252 | 1-(3-chlorophenyl)-N-((3-(hydroxymethyl)oxetan-3-yl)methyl)-6-(2-methyl-1-oxo- |
| | 1,2,3,4-tetrahydroisoquinolin-7-yl)-7-oxo-4,5,6,7-tetrahydro-1H-pyrazolo[3,4- |
| | c]pyridine-3-carboxamide |
| 1253 | 1-(3-chlorophenyl)-N-(cyclopropylmethyl)-6-(2-(2-fluoroethyl)-1-oxo-1,2,3,4- |
| | tetrahydroisoquinolin-7-yl)-7-oxo-4,5,6,7-tetrahydro-1H-pyrazolo[3,4-c]pyridine-3- |
| | carboxamide |
| 1254 | 1-(3-chlorophenyl)-N-((1,5-dimethyl-1H-pyrazol-3-yl)methyl)-6-(2-(2-fluoroethyl)-1- |
| | oxo-1,2,3,4-tetrahydroisoquinolin-7-yl)-7-oxo-4,5,6,7-tetrahydro-1H-pyrazolo[3,4- |
| | c]pyridine-3-carboxamide |
| 1255 | 1-(3-chlorophenyl)-6-(2-(2-hydroxyethyl)-1-oxo-1,2,3,4-tetrahydroisoquinolin-7-yl)-N- |
| | ((5-methyl-1H-pyrazol-3-yl)methyl)-7-oxo-4,5,6,7-tetrahydro-1H-pyrazolo[3,4- |
| | c]pyridine-3-carboxamide |
| 1256 | 1-(3-chlorophenyl)-N-((4,6-dimethylpyridin-3-yl)methyl)-6-(2-(2-hydroxyethyl)-1-oxo- |
| | 1,2,3,4-tetrahydroisoquinolin-7-yl)-7-oxo-4,5,6,7-tetrahydro-1H-pyrazolo[3,4- |
| | c]pyridine-3-carboxamide |
| 1257 | 1-(3-chlorophenyl)-N-(cyclopropylmethyl)-7-oxo-6-(1-oxo-2-((tetrahydrofuran-3- |
| | yl)methyl)-1,2,3,4-tetrahydroisoquinolin-7-yl)-4,5,6,7-tetrahydro-1H-pyrazolo[3,4- |
| | c]pyridine-3-carboxamide |
| 1258 | 1-(3-chlorophenyl)-N-((1,5-dimethyl-1H-pyrazol-3-yl)methyl)-7-oxo-6-(1-oxo-2- |
| | ((tetrahydrofuran-3-yl)methyl)-1,2,3,4-tetrahydroisoquinolin-7-yl)-4,5,6,7-tetrahydro- |
| | 1H-pyrazolo[3,4-c]pyridine-3-carboxamide |
| 1259 | 1-(3-chlorophenyl)-N-((5-cyclopropyl-1H-pyrazol-3-yl)methyl)-6-(2-(2-hydroxyethyl)-1- |
| | oxo-1,2,3,4-tetrahydroisoquinolin-7-yl)-7-oxo-4,5,6,7-tetrahydro-1H-pyrazolo[3,4- |
| | c]pyridine-3-carboxamide |
| 1260 | 1-(3-chlorophenyl)-6-(2-(2-hydroxyethyl)-1-oxo-1,2,3,4-tetrahydroisoquinolin-7-yl)-7- |
| | oxo-N-((tetrahydrofuran-3-yl)methyl)-4,5,6,7-tetrahydro-1H-pyrazolo[3,4-c]pyridine- |
| | 3-carboxamide |
| 1261 | 1-(3-chlorophenyl)-N-(cyclopropylmethyl)-6-(2-(2-methoxyethyl)-1-oxo-1,2,3,4- |
| | tetrahydroisoquinolin-7-yl)-7-oxo-4,5,6,7-tetrahydro-1H-pyrazolo[3,4-c]pyridine-3- |
| | carboxamide |
| 1262 | 1-(3-chlorophenyl)-N-((1,5-dimethyl-1H-pyrazol-3-yl)methyl)-6-(2-(2-methoxyethyl)-1- |
| | oxo-1,2,3,4-tetrahydroisoquinolin-7-yl)-7-oxo-4,5,6,7-tetrahydro-1H-pyrazolo[3,4- |
| | c]pyridine-3-carboxamide |
| 1263 | 1-(3-chlorophenyl)-N-(cyclopropylmethyl)-7-oxo-6-(3-(pyrrolidine-1-carbonyl)phenyl)- |
| | 4,5,6,7-tetrahydro-1H-pyrazolo[3,4-c]pyridine-3-carboxamide |
| 1264 | 1-(3-chlorophenyl)-N-((1,3-dimethyl-1H-pyrazol-5-yl)methyl)-6-(2-methyl-1-oxo- |
| | 1,2,3,4-tetrahydroisoquinolin-7-yl)-7-oxo-4,5,6,7-tetrahydro-1H-pyrazolo[3,4- |
| | c]pyridine-3-carboxamide |
| 1265 | 1-(3-chlorophenyl)-N-((3R,4S)-4-methoxytetrahydro-2H-pyran-3-yl)-6-(2-methyl-1- |
| | oxo-1,2,3,4-tetrahydroisoquinolin-7-yl)-7-oxo-4,5,6,7-tetrahydro-1H-pyrazolo[3,4- |
| | c]pyridine-3-carboxamide |
| 1266 | 1-(3-chlorophenyl)-6-(2-methyl-1-oxo-1,2,3,4-tetrahydroisoquinolin-7-yl)-N-(4- |
| | methyltetrahydro-2H-pyran-4-yl)-7-oxo-4,5,6,7-tetrahydro-1H-pyrazolo[3,4- |
| | c]pyridine-3-carboxamide |
| 1267 | 1-(3-chlorophenyl)-6-(2-methyl-1-oxo-1,2,3,4-tetrahydroisoquinolin-7-yl)-N-((6- |
| | methylpyridazin-3-yl)methyl)-7-oxo-4,5,6,7-tetrahydro-1H-pyrazolo[3,4-c]pyridine-3- |
| | carboxamide |
| 1268 | 1-(3-chlorophenyl)-N-((3,5-dimethyl-1H-pyrazol-4-yl)methyl)-6-(2-methyl-1-oxo- |
| | 1,2,3,4-tetrahydroisoquinolin-7-yl)-7-oxo-4,5,6,7-tetrahydro-1H-pyrazolo[3,4- |
| | c]pyridine-3-carboxamide |
| 1269 | 1-(3-chlorophenyl)-N-(cyclopropylmethyl)-6-(3-(N-methylacetamido)phenyl)-7-oxo- |
| | 4,5,6,7-tetrahydro-1H-pyrazolo[3,4-c]pyridine-3-carboxamide |
| 1270 | 1-(3-chlorophenyl)-6-(3-(cyclopropyl(methyl)carbamoyl)phenyl)-N- |
| | (cyclopropylmethyl)-7-oxo-4,5,6,7-tetrahydro-1H-pyrazolo[3,4-c]pyridine-3- |
| | carboxamide |
| 1271 | 1-(3-chlorophenyl)-N-((4-methoxypyridin-3-yl)methyl)-6-(2-methyl-1-oxo-1,2,3,4- |
| | tetrahydroisoquinolin-7-yl)-7-oxo-4,5,6,7-tetrahydro-1H-pyrazolo[3,4-c]pyridine-3- |
| | carboxamide |
| 1272 | 7-(1-(3-chlorophenyl)-3-(morpholine-4-carbonyl)-7-oxo-1,4,5,7-tetrahydro-6H- |
| | pyrazolo[3,4-c]pyridin-6-yl)-2-methyl-3,4-dihydroisoquinolin-1(2H)-one |
| 1273 | 1-(3-chlorophenyl)-N-(cyclopropylmethyl)-6-(4-(dimethylcarbamoyl)pyridin-2-yl)-7- |
| | oxo-4,5,6,7-tetrahydro-1H-pyrazolo[3,4-c]pyridine-3-carboxamide |
| 1274 | 1-(3-chlorophenyl)-N-(((1r,3r)-3-hydroxycyclobutyl)methyl)-6-(2-methyl-1-oxo-1,2,3,4- |
| | tetrahydroisoquinolin-7-yl)-7-oxo-4,5,6,7-tetrahydro-1H-pyrazolo[3,4-c]pyridine-3- |
| | carboxamide |
| 1275 | 1-(3-chlorophenyl)-N-((1,5-dimethyl-1H-pyrazol-3-yl)methyl)-6-(2-(3-hydroxypropyl)- |
| | 1-oxo-1,2,3,4-tetrahydroisoquinolin-7-yl)-7-oxo-4,5,6,7-tetrahydro-1H-pyrazolo[3,4- |
| | c]pyridine-3-carboxamide |
| 1276 | 1-(3-chlorophenyl)-N-((1,5-dimethyl-1H-pyrazol-3-yl)methyl)-7-oxo-6-(1,3,3-trimethyl- |
| | 2-oxoindolin-6-yl)-4,5,6,7-tetrahydro-1H-pyrazolo[3,4-c]pyridine-3-carboxamide |
| 1277 | 1-(3-chlorophenyl)-N-(cyclopropylmethyl)-7-oxo-6-(1,3,3-trimethyl-2-oxoindolin-6-yl)- |
| | 4,5,6,7-tetrahydro-1H-pyrazolo[3,4-c]pyridine-3-carboxamide |
| 1278 | 1-(3-chlorophenyl)-N-(cyclopropylmethyl)-6-(2-(dimethylcarbamoyl)pyridin-4-yl)-7- |
| | oxo-4,5,6,7-tetrahydro-1H-pyrazolo[3,4-c]pyridine-3-carboxamide |
| 1279 | 1-(3-chlorophenyl)-N-(cyclopropylmethyl)-6-(5-(dimethylcarbamoyl)pyridin-3-yl)-7- |
| | oxo-4,5,6,7-tetrahydro-1H-pyrazolo[3,4-c]pyridine-3-carboxamide |
| 1280 | 1-(3-chlorophenyl)-N-((4-(hydroxymethyl)-5-oxabicyclo[2.1.1]hexan-1-yl)methyl)-6-(2- |
| | methyl-1-oxo-1,2,3,4-tetrahydroisoquinolin-7-yl)-7-oxo-4,5,6,7-tetrahydro-1H- |
| | pyrazolo[3,4-c]pyridine-3-carboxamide |
| 1281 | 1-(3-chlorophenyl)-N-(cyclopropylmethyl)-6-(3-(dimethylcarbamoyl)-4-fluorophenyl)- |
| | 7-oxo-4,5,6,7-tetrahydro-1H-pyrazolo[3,4-c]pyridine-3-carboxamide |
| 1282 | 1-(3-chlorophenyl)-6-(2-methyl-1-oxo-1,2,3,4-tetrahydroisoquinolin-7-yl)-N-((5- |
| | methylpyridazin-3-yl)methyl)-7-oxo-4,5,6,7-tetrahydro-1H-pyrazolo[3,4-c]pyridine-3- |
| | carboxamide |
| 1283 | 1-(3-chlorophenyl)-N-((5-(hydroxymethyl)-2-oxabicyclo[3.1.1]heptan-1-yl)methyl)-6- |
| | (2-methyl-1-oxo-1,2,3,4-tetrahydroisoquinolin-7-yl)-7-oxo-4,5,6,7-tetrahydro-1H- |
| | pyrazolo[3,4-c]pyridine-3-carboxamide |
| 1284 | ethyl 1-(3-chlorophenyl)-6-(2-methyl-1-oxo-1,2,3,4-tetrahydroisoquinolin-7-yl)-7-oxo- |
| | 4,5,6,7-tetrahydro-1H-pyrazolo[3,4-c]pyridine-3-carboxylate |
| 1285 | 1-(3-chlorophenyl)-N-(cyclopropylmethyl)-7-oxo-6-(3-(2-oxopiperidin-1-yl)phenyl)- |
| | 4,5,6,7-tetrahydro-1H-pyrazolo[3,4-c]pyridine-3-carboxamide |
| 1286 | 1-(3-chlorophenyl)-N-(cyclopropylmethyl)-6-(3-(diethylcarbamoyl)phenyl)-7-oxo- |
| | 4,5,6,7-tetrahydro-1H-pyrazolo[3,4-c]pyridine-3-carboxamide |
| 1287 | 1-(3-chlorophenyl)-N-((4-methylpyridin-3-yl)methyl)-7-oxo-6-(3-(2-oxopiperidin-1- |
| | yl)phenyl)-4,5,6,7-tetrahydro-1H-pyrazolo[3,4-c]pyridine-3-carboxamide |
| 1288 | 1-(3-chlorophenyl)-N-((1,5-dimethyl-1H-pyrazol-3-yl)methyl)-6-(4-(2-hydroxyethyl)-5- |
| | oxo-2,3,4,5-tetrahydrobenzo[f][1,4]oxazepin-7-yl)-7-oxo-4,5,6,7-tetrahydro-1H- |
| | pyrazolo[3,4-c]pyridine-3-carboxamide |
| 1289 | 1-(3-chlorophenyl)-N-(cyclopropylmethyl)-6-(4-(2-hydroxyethyl)-5-oxo-2,3,4,5- |
| | tetrahydrobenzo[f][1,4]oxazepin-7-yl)-7-oxo-4,5,6,7-tetrahydro-1H-pyrazolo[3,4- |
| | c]pyridine-3-carboxamide |
| 1290 | 1-(3-chlorophenyl)-N-((1,5-dimethyl-1H-pyrazol-3-yl)methyl)-6-(2-(oxetan-3-yl)-1-oxo- |
| | 1,2,3,4-tetrahydroisoquinolin-7-yl)-7-oxo-4,5,6,7-tetrahydro-1H-pyrazolo[3,4- |
| | c]pyridine-3-carboxamide |
| 1292 | 1-(3-chlorophenyl)-N-(cyclopropylmethyl)-7-oxo-6-(1-oxo-2-((tetrahydrofuran-2- |
| | yl)methyl)-1,2,3,4-tetrahydroisoquinolin-7-yl)-4,5,6,7-tetrahydro-1H-pyrazolo[3,4- |
| | c]pyridine-3-carboxamide |
| 1293 | 1-(3-chlorophenyl)-N-((1,5-dimethyl-1H-pyrazol-3-yl)methyl)-7-oxo-6-(1-oxo-2- |
| | ((tetrahydrofuran-2-yl)methyl)-1,2,3,4-tetrahydroisoquinolin-7-yl)-4,5,6,7-tetrahydro- |
| | 1H-pyrazolo[3,4-c]pyridine-3-carboxamide |
| 1294 | 1-(3-chlorophenyl)-N-(cyclopropylmethyl)-6-(2-(2-hydroxyethyl)-1-oxo-2,3,4,5- |
| | tetrahydro-1H-benzo[c]azepin-8-yl)-7-oxo-4,5,6,7-tetrahydro-1H-pyrazolo[3,4- |
| | c]pyridine-3-carboxamide |
| 1295 | 1-(3-chlorophenyl)-N-(cyclopropylmethyl)-6-(4-(dimethylcarbamoyl)thiophen-2-yl)-7- |
| | oxo-4,5,6,7-tetrahydro-1H-pyrazolo[3,4-c]pyridine-3-carboxamide |
| 1296 | 1-(3-chlorophenyl)-N-((1-isopropyl-1H-pyrazol-3-yl)methyl)-6-(2-methyl-1-oxo-1,2,3,4- |
| | tetrahydroisoquinolin-7-yl)-7-oxo-4,5,6,7-tetrahydro-1H-pyrazolo[3,4-c]pyridine-3- |
| | carboxamide |
| 1297 | 1-(3-chlorophenyl)-N-ethyl-6-(2-methyl-1-oxo-1,2,3,4-tetrahydroisoquinolin-7-yl)-7- |
| | oxo-4,5,6,7-tetrahydro-1H-pyrazolo[3,4-c]pyridine-3-carboxamide |
| 1298 | 1-(3-chlorophenyl)-N-(cyclopropylmethyl)-6-(5-(dimethylcarbamoyl)pyridazin-3-yl)-7- |
| | oxo-4,5,6,7-tetrahydro-1H-pyrazolo[3,4-c]pyridine-3-carboxamide |
| 1299 | 1-(3-chlorophenyl)-6-(4-(2-hydroxyethyl)-5-oxo-2,3,4,5- |
| | tetrahydrobenzo[f][1,4]oxazepin-7-yl)-N-((4-methylpyridin-3-yl)methyl)-7-oxo-4,5,6,7- |
| | tetrahydro-1H-pyrazolo[3,4-c]pyridine-3-carboxamide |
| 1300 | 1-(3-chlorophenyl)-N-(cyclopropylmethyl)-6-(4,4-difluoro-2-(2-hydroxyethyl)-1,2,3,4- |
| | tetrahydroisoquinolin-7-yl)-7-oxo-4,5,6,7-tetrahydro-1H-pyrazolo[3,4-c]pyridine-3- |
| | carboxamide |
| 1301 | 1-(3-chlorophenyl)-N-(cyclopropylmethyl)-6-(6-methyl-5-oxo-5,6,7,8-tetrahydro-1,6- |
| | naphthyridin-3-yl)-7-oxo-4,5,6,7-tetrahydro-1H-pyrazolo[3,4-c]pyridine-3- |
| | carboxamide |
| 1302 | 1-(3-chlorophenyl)-6-(6-methyl-5-oxo-5,6,7,8-tetrahydro-1,6-naphthyridin-3-yl)-N-((4- |
| | methylpyridin-3-yl)methyl)-7-oxo-4,5,6,7-tetrahydro-1H-pyrazolo[3,4-c]pyridine-3- |
| | carboxamide |
| 1303 | 1-(3-chlorophenyl)-N-((1,5-dimethyl-1H-pyrazol-3-yl)methyl)-6-(6-methyl-5-oxo- |
| | 5,6,7,8-tetrahydro-1,6-naphthyridin-3-yl)-7-oxo-4,5,6,7-tetrahydro-1H-pyrazolo[3,4- |
| | c]pyridine-3-carboxamide |
| 1304 | 6-(1-acetylindolin-6-yl)-1-(3-chlorophenyl)-N-((1,5-dimethyl-1H-pyrazol-3-yl)methyl)- |
| | 7-oxo-4,5,6,7-tetrahydro-1H-pyrazolo[3,4-c]pyridine-3-carboxamide |
| 1305 | 1-(3-chlorophenyl)-N-((1,5-dimethyl-1H-pyrazol-3-yl)methyl)-6-(2-(2-hydroxyethyl)-1- |
| | oxo-2,3,4,5-tetrahydro-1H-benzo[c]azepin-8-yl)-7-oxo-4,5,6,7-tetrahydro-1H- |
| | pyrazolo[3,4-c]pyridine-3-carboxamide |
| 1306 | 1-(3-chlorophenyl)-6-(2-(2-hydroxyethyl)-1-oxo-2,3,4,5-tetrahydro-1H-benzo[c]azepin- |
| | 8-yl)-N-((4-methylpyridin-3-yl)methyl)-7-oxo-4,5,6,7-tetrahydro-1H-pyrazolo[3,4- |
| | c]pyridine-3-carboxamide |
| 1307 | (S)-1-(3-chlorophenyl)-N-(2-hydroxy-1-phenylethyl)-6-(2-methyl-1-oxo-1,2,3,4- |
| | tetrahydroisoquinolin-7-yl)-7-oxo-4,5,6,7-tetrahydro-1H-pyrazolo[3,4-c]pyridine-3- |
| | carboxamide |
| 1308 | 1-(3-chlorophenyl)-6-(2-methyl-1-oxo-1,2,3,4-tetrahydroisoquinolin-7-yl)-N-((3- |
| | methylpyridin-2-yl)methyl)-7-oxo-4,5,6,7-tetrahydro-1H-pyrazolo[3,4-c]pyridine-3- |
| | carboxamide |
| 1309 | 1-(3-chlorophenyl)-6-(2-methyl-1-oxo-1,2,3,4-tetrahydroisoquinolin-7-yl)-N-((2- |
| | methylpyridin-3-yl)methyl)-7-oxo-4,5,6,7-tetrahydro-1H-pyrazolo[3,4-c]pyridine-3- |
| | carboxamide |
| 1310 | 1-(3-chlorophenyl)-6-(2-methyl-1-oxo-1,2,3,4-tetrahydroisoquinolin-7-yl)-N-((4- |
| | methylpyridazin-3-yl)methyl)-7-oxo-4,5,6,7-tetrahydro-1H-pyrazolo[3,4-c]pyridine-3- |
| | carboxamide |
| 1311 | 1-(3-chlorophenyl)-N-(cyclopropylmethyl)-6-(3-((2- |
| | methoxyethyl)(methyl)carbamoyl)phenyl)-7-oxo-4,5,6,7-tetrahydro-1H-pyrazolo[3,4- |
| | c]pyridine-3-carboxamide |
| 1312 | 1-(3-chlorophenyl)-N-(cyclopropylmethyl)-6-(3-((2- |
| | hydroxyethyl)(methyl)carbamoyl)phenyl)-7-oxo-4,5,6,7-tetrahydro-1H-pyrazolo[3,4- |
| | c]pyridine-3-carboxamide |
| 1313 | 1-(3-chlorophenyl)-N-(cyclopropylmethyl)-7-oxo-6-(3-(3-oxomorpholino)phenyl)- |
| | 4,5,6,7-tetrahydro-1H-pyrazolo[3,4-c]pyridine-3-carboxamide |
| 1314 | 6-(1-acetyl-1,2,3,4-tetrahydroquinolin-7-yl)-1-(3-chlorophenyl)-N-((4-methylpyridin-3- |
| | yl)methyl)-7-oxo-4,5,6,7-tetrahydro-1H-pyrazolo[3,4-c]pyridine-3-carboxamide |
| 1316 | 1-(3-chlorophenyl)-N-(cyclopropylmethyl)-7-oxo-6-(3-((2-oxooxazolidin-3- |
| | yl)methyl)phenyl)-4,5,6,7-tetrahydro-1H-pyrazolo[3,4-c]pyridine-3-carboxamide |
| 1317 | 1-(3-chlorophenyl)-N-((1,5-dimethyl-1H-pyrazol-3-yl)methyl)-6-(4-ethyl-5-oxo-2,3,4,5- |
| | tetrahydrobenzo[f][1,4]oxazepin-7-yl)-7-oxo-4,5,6,7-tetrahydro-1H-pyrazolo[3,4- |
| | c]pyridine-3-carboxamide |
| 1318 | 1-(3-chlorophenyl)-6-(4-ethyl-5-oxo-2,3,4,5-tetrahydrobenzo[f][1,4]oxazepin-7-yl)-N- |
| | ((4-methylpyridin-3-yl)methyl)-7-oxo-4,5,6,7-tetrahydro-1H-pyrazolo[3,4-c]pyridine-3- |
| | carboxamide |
| 1319 | 1-(3-chlorophenyl)-N-((1,5-dimethyl-1H-pyrazol-3-yl)methyl)-6-(4-methyl-5-oxo- |
| | 2,3,4,5-tetrahydrobenzo[f][1,4]oxazepin-7-yl)-7-oxo-4,5,6,7-tetrahydro-1H- |
| | pyrazolo[3,4-c]pyridine-3-carboxamide |
| 1320 | 1-(3-chlorophenyl)-N-((1,5-dimethyl-1H-pyrazol-3-yl)methyl)-6-(4-isopropyl-5-oxo- |
| | 2,3,4,5-tetrahydrobenzo[f][1,4]oxazepin-7-yl)-7-oxo-4,5,6,7-tetrahydro-1H- |
| | pyrazolo[3,4-c]pyridine-3-carboxamide |
| 1321 | 6-(2-(2-amino-2-oxoethyl)-1-oxo-1,2,3,4-tetrahydroisoquinolin-7-yl)-1-(3- |
| | chlorophenyl)-N-((5-methyl-1H-pyrazol-3-yl)methyl)-7-oxo-4,5,6,7-tetrahydro-1H- |
| | pyrazolo[3,4-c]pyridine-3-carboxamide |
| 1322 | ethyl 1-(3-chlorophenyl)-6-(3-((2-hydroxy-N-methylacetamido)methyl)phenyl)-7-oxo- |
| | 4,5,6,7-tetrahydro-1H-pyrazolo[3,4-c]pyridine-3-carboxylate |
| 1323 | ethyl 1-(3-chlorophenyl)-7-oxo-6-(3-(2-oxopyrrolidin-1-yl)phenyl)-4,5,6,7-tetrahydro- |
| | 1H-pyrazolo[3,4-c]pyridine-3-carboxylate |
| 1324 | ethyl 1-(3-chlorophenyl)-6-(3-(dimethylcarbamoyl)-4-methoxyphenyl)-7-oxo-4,5,6,7- |
| | tetrahydro-1H-pyrazolo[3,4-c]pyridine-3-carboxylate |
| 1325 | methyl 2-(1-(3-chlorophenyl)-6-(2-methyl-1-oxo-1,2,3,4-tetrahydroisoquinolin-7-yl)-7- |
| | oxo-4,5,6,7-tetrahydro-1H-pyrazolo[3,4-c]pyridine-3-carboxamido)-2-(pyridin-3- |
| | yl)acetate |
| 1326 | 1-(3-chlorophenyl)-N-((2,4-dimethylpyridin-3-yl)methyl)-6-(2-(2-hydroxyethyl)-1-oxo- |
| | 1,2,3,4-tetrahydroisoquinolin-7-yl)-7-oxo-4,5,6,7-tetrahydro-1H-pyrazolo[3,4- |
| | c]pyridine-3-carboxamide |
| 1327 | 1-(3-chlorophenyl)-6-(2-(2-hydroxyethyl)-1-oxo-1,2,3,4-tetrahydroisoquinolin-7-yl)-N- |
| | (1-(4-methylpyridin-3-yl)ethyl)-7-oxo-4,5,6,7-tetrahydro-1H-pyrazolo[3,4-c]pyridine-3- |
| | carboxamide |
| 1328 | 2-(1-(3-chlorophenyl)-6-(2-methyl-1-oxo-1,2,3,4-tetrahydroisoquinolin-7-yl)-7-oxo- |
| | 4,5,6,7-tetrahydro-1H-pyrazolo[3,4-c]pyridine-3-carboxamido)-2-(pyridin-3-yl)acetic |
| | acid |
| 1329 | (R)-1-(3-chlorophenyl)-N-((1,5-dimethyl-1H-pyrazol-3-yl)methyl)-6-(4-(2- |
| | hydroxyethyl)-3-methyl-5-oxo-2,3,4,5-tetrahydrobenzo[f][1,4]oxazepin-7-yl)-7-oxo- |
| | 4,5,6,7-tetrahydro-1H-pyrazolo[3,4-c]pyridine-3-carboxamide |
| 1330 | 2-(7-(1-(3-chlorophenyl)-3-((cyclopropylmethyl)carbamoyl)-7-oxo-1,4,5,7-tetrahydro- |
| | 6H-pyrazolo[3,4-c]pyridin-6-yl)-1-oxo-3,4-dihydroisoquinolin-2(1H)-yl)acetic acid |
| 1331 | 2-(7-(1-(3-chlorophenyl)-3-(((1,5-dimethyl-1H-pyrazol-3-yl)methyl)carbamoyl)-7-oxo- |
| | 1,4,5,7-tetrahydro-6H-pyrazolo[3,4-c]pyridin-6-yl)-1-oxo-3,4-dihydroisoquinolin- |
| | 2(1H)-yl)acetic acid |
| 1332 | 2-(7-(1-(3-chlorophenyl)-3-(((5-methyl-1H-pyrazol-3-yl)methyl)carbamoyl)-7-oxo- |
| | 1,4,5,7-tetrahydro-6H-pyrazolo[3,4-c]pyridin-6-yl)-1-oxo-3,4-dihydroisoquinolin- |
| | 2(1H)-yl)acetic acid |
| 1333 | 6-(2-(2-amino-2-oxoethyl)-1-oxo-1,2,3,4-tetrahydroisoquinolin-7-yl)-1-(3- |
| | chlorophenyl)-N-(cyclopropylmethyl)-7-oxo-4,5,6,7-tetrahydro-1H-pyrazolo[3,4- |
| | c]pyridine-3-carboxamide |
| 1334 | 1-(3-chlorophenyl)-N-methyl-6-(2-methyl-1-oxo-1,2,3,4-tetrahydroisoquinolin-7-yl)-7- |
| | oxo-4,5,6,7-tetrahydro-1H-pyrazolo[3,4-c]pyridine-3-carboxamide |
| 1335 | 7-(3-(azetidine-1-carbonyl)-1-(3-chlorophenyl)-7-oxo-1,4,5,7-tetrahydro-6H- |
| | pyrazolo[3,4-c]pyridin-6-yl)-2-methyl-3,4-dihydroisoquinolin-1(2H)-one |
| 1336 | (S)-1-(3-chlorophenyl)-6-(2-methyl-1-oxo-1,2,3,4-tetrahydroisoquinolin-7-yl)-7-oxo-N- |
| | (tetrahydrofuran-3-yl)-4,5,6,7-tetrahydro-1H-pyrazolo[3,4-c]pyridine-3-carboxamide |
| 1337 | (R)-1-(3-chlorophenyl)-N-(1-hydroxypropan-2-yl)-6-(2-methyl-1-oxo-1,2,3,4- |
| | tetrahydroisoquinolin-7-yl)-7-oxo-4,5,6,7-tetrahydro-1H-pyrazolo[3,4-c]pyridine-3- |
| | carboxamide |
| 1338 | (R)-1-(3-chlorophenyl)-6-(2-methyl-1-oxo-1,2,3,4-tetrahydroisoquinolin-7-yl)-7-oxo-N- |
| | (tetrahydrofuran-3-yl)-4,5,6,7-tetrahydro-1H-pyrazolo[3,4-c]pyridine-3-carboxamide |
| 1339 | 1-(3-chlorophenyl)-6-(2-methyl-1-oxo-1,2,3,4-tetrahydroisoquinolin-7-yl)-N-(oxetan-3- |
| | ylmethyl)-7-oxo-4,5,6,7-tetrahydro-1H-pyrazolo[3,4-c]pyridine-3-carboxamide |
| 1340 | 1-(3-chlorophenyl)-N-(2-hydroxyethyl)-6-(2-methyl-1-oxo-1,2,3,4- |
| | tetrahydroisoquinolin-7-yl)-7-oxo-4,5,6,7-tetrahydro-1H-pyrazolo[3,4-c]pyridine-3- |
| | carboxamide |
| 1341 | (1-(3-chlorophenyl)-6-(2-methyl-1-oxo-1,2,3,4-tetrahydroisoquinolin-7-yl)-7-oxo- |
| | 4,5,6,7-tetrahydro-1H-pyrazolo[3,4-c]pyridine-3-carbonyl)glycine |
| 1342 | 1-(3-chlorophenyl)-6-(2-methyl-1-oxo-1,2,3,4-tetrahydroisoquinolin-7-yl)-N-(oxetan-3- |
| | yl)-7-oxo-4,5,6,7-tetrahydro-1H-pyrazolo[3,4-c]pyridine-3-carboxamide |
| 1343 | 7-(1-(3-chlorophenyl)-3-(isoxazolidine-2-carbonyl)-7-oxo-1,4,5,7-tetrahydro-6H- |
| | pyrazolo[3,4-c]pyridin-6-yl)-2-methyl-3,4-dihydroisoquinolin-1(2H)-one |
| 1344 | (S)-1-(3-chlorophenyl)-N-(1-hydroxypropan-2-yl)-6-(2-methyl-1-oxo-1,2,3,4- |
| | tetrahydroisoquinolin-7-yl)-7-oxo-4,5,6,7-tetrahydro-1H-pyrazolo[3,4-c]pyridine-3- |
| | carboxamide |
| 1345 | 1-(3-chlorophenyl)-6-(2-methyl-1-oxo-1,2,3,4-tetrahydroisoquinolin-7-yl)-N- |
| | (methylsulfonyl)-7-oxo-4,5,6,7-tetrahydro-1H-pyrazolo[3,4-c]pyridine-3-carboxamide |
| 1346 | 1-(3-chlorophenyl)-N-cyclobutyl-6-(2-methyl-1-oxo-1,2,3,4-tetrahydroisoquinolin-7- |
| | yl)-7-oxo-4,5,6,7-tetrahydro-1H-pyrazolo[3,4-c]pyridine-3-carboxamide |
| 1347 | 1-(3-chlorophenyl)-N-hydroxy-6-(2-methyl-1-oxo-1,2,3,4-tetrahydroisoquinolin-7-yl)- |
| | 7-oxo-4,5,6,7-tetrahydro-1H-pyrazolo[3,4-c]pyridine-3-carboxamide |
| 1350 | ethyl 1-(3-chlorophenyl)-7-oxo-6-(3-(2-oxopyridin-1(2H)-yl)phenyl)-4,5,6,7-tetrahydro- |
| | 1H-pyrazolo[3,4-c]pyridine-3-carboxylate |
| 1351 | (R)-1-(3-chlorophenyl)-N-((1,5-dimethyl-1H-pyrazol-3-yl)methyl)-6-(3- |
| | (hydroxymethyl)-4-methyl-5-oxo-2,3,4,5-tetrahydrobenzo[f][1,4]oxazepin-7-yl)-7-oxo- |
| | 4,5,6,7-tetrahydro-1H-pyrazolo[3,4-c]pyridine-3-carboxamide |
| 1352 | 1-(3-chlorophenyl)-6-(4-ethyl-5-oxo-2,3,4,5-tetrahydrobenzo[f][1,4]oxazepin-7-yl)-N- |
| | ((5-methyl-1H-pyrazol-3-yl)methyl)-7-oxo-4,5,6,7-tetrahydro-1H-pyrazolo[3,4- |
| | c]pyridine-3-carboxamide |
| 1353 | 7-(1-(3-chlorophenyl)-3-(4,5-dihydrooxazol-2-yl)-7-oxo-1,4,5,7-tetrahydro-6H- |
| | pyrazolo[3,4-c]pyridin-6-yl)-2-methyl-3,4-dihydroisoquinolin-1(2H)-one |
| 1354 | 7-(1-(3-chlorophenyl)-3-(((3-methyloxetan-3-yl)oxy)methyl)-7-oxo-1,4,5,7-tetrahydro- |
| | 6H-pyrazolo[3,4-c]pyridin-6-yl)-2-methyl-3,4-dihydroisoquinolin-1(2H)-one |
| 1355 | 1-(3-chlorophenyl)-N-((1-cyclopropyl-1H-pyrazol-3-yl)methyl)-6-(2-(2-hydroxyethyl)-1- |
| | oxo-1,2,3,4-tetrahydroisoquinolin-7-yl)-7-oxo-4,5,6,7-tetrahydro-1H-pyrazolo[3,4- |
| | c]pyridine-3-carboxamide |
| 1356 | 7-(1-(3-chlorophenyl)-3-(((4-methylpyridin-3-yl)methoxy)methyl)-7-oxo-1,4,5,7- |
| | tetrahydro-6H-pyrazolo[3,4-c]pyridin-6-yl)-2-methyl-3,4-dihydroisoquinolin-1(2H)-one |
| 1357 | 1-(3-chlorophenyl)-6-(2-(2-hydroxyethyl)-1-oxo-1,2,3,4-tetrahydroisoquinolin-7-yl)-N- |
| | ((3-methylpyridin-4-yl)methyl)-7-oxo-4,5,6,7-tetrahydro-1H-pyrazolo[3,4-c]pyridine-3- |
| | carboxamide |
| 1358 | 2-((1-(3-chlorophenyl)-6-(2-methyl-1-oxo-1,2,3,4-tetrahydroisoquinolin-7-yl)-7-oxo- |
| | 4,5,6,7-tetrahydro-1H-pyrazolo[3,4-c]pyridin-3-yl)methoxy)acetic acid |
| 1359 | 1-(3-chlorophenyl)-6-(2-methyl-1-oxo-1,2,3,4-tetrahydroisoquinolin-7-yl)-N-(1- |
| | methylcyclopropyl)-7-oxo-4,5,6,7-tetrahydro-1H-pyrazolo[3,4-c]pyridine-3- |
| | carboxamide |
| 1360 | 1-(3-chlorophenyl)-N-(1-(hydroxymethyl)cyclopropyl)-6-(2-methyl-1-oxo-1,2,3,4- |
| | tetrahydroisoquinolin-7-yl)-7-oxo-4,5,6,7-tetrahydro-1H-pyrazolo[3,4-c]pyridine-3- |
| | carboxamide |
| 1361 | 1-(1-(3-chlorophenyl)-6-(2-methyl-1-oxo-1,2,3,4-tetrahydroisoquinolin-7-yl)-7-oxo- |
| | 4,5,6,7-tetrahydro-1H-pyrazolo[3,4-c]pyridine-3-carboxamido)cyclopropane-1- |
| | carboxylic acid |
| 1362 | 1-(3-chlorophenyl)-N-ethyl-7-oxo-6-(3-(2-oxooxazolidin-3-yl)phenyl)-4,5,6,7- |
| | tetrahydro-1H-pyrazolo[3,4-c]pyridine-3-carboxamide |
| 1363 | 1-(3-chlorophenyl)-N-ethyl-7-oxo-6-(3-(2-oxo-1,3-oxazinan-3-yl)phenyl)-4,5,6,7- |
| | tetrahydro-1H-pyrazolo[3,4-c]pyridine-3-carboxamide |
| 1364 | 1-(3-chlorophenyl)-N-ethyl-6-(3-(3-methyl-2-oxoimidazolidin-1-yl)phenyl)-7-oxo- |
| | 4,5,6,7-tetrahydro-1H-pyrazolo[3,4-c]pyridine-3-carboxamide |
| 1365 | 1-(3-chlorophenyl)-N-ethyl-7-oxo-6-(3-(2-oxopyrrolidin-1-yl)phenyl)-4,5,6,7- |
| | tetrahydro-1H-pyrazolo[3,4-c]pyridine-3-carboxamide |
| 1366 | 1-(3-chlorophenyl)-6-(3-(1,1-dioxidoisothiazolidin-2-yl)phenyl)-N-ethyl-7-oxo-4,5,6,7- |
| | tetrahydro-1H-pyrazolo[3,4-c]pyridine-3-carboxamide |
| 1367 | 1-(3-chlorophenyl)-N-ethyl-7-oxo-6-(3-(3-oxo-2-azabicyclo[3.1.0]hexan-2-yl)phenyl)- |
| | 4,5,6,7-tetrahydro-1H-pyrazolo[3,4-c]pyridine-3-carboxamide |
| 1368 | 1-(3-chlorophenyl)-N-ethyl-7-oxo-6-(3-(2-oxo-3-azabicyclo[3.1.0]hexan-3-yl)phenyl)- |
| | 4,5,6,7-tetrahydro-1H-pyrazolo[3,4-c]pyridine-3-carboxamide |
| 1369 | 1-(3-chlorophenyl)-N-ethyl-6-(3-(4-methyl-5-oxo-4,6-diazaspiro[2.4]heptan-6- |
| | yl)phenyl)-7-oxo-4,5,6,7-tetrahydro-1H-pyrazolo[3,4-c]pyridine-3-carboxamide |
| 1370 | 1-(3-chlorophenyl)-6-(1,4-dimethyl-2,5-dioxo-2,3,4,5-tetrahydro-1H- |
| | benzo[e][1,4]diazepin-7-yl)-N-((5-methyl-1H-pyrazol-3-yl)methyl)-7-oxo-4,5,6,7- |
| | tetrahydro-1H-pyrazolo[3,4-c]pyridine-3-carboxamide |
| 1371 | 1-(3-chlorophenyl)-N-ethyl-6-(4-ethyl-5-oxo-2,3,4,5-tetrahydrobenzo[f][1,4]oxazepin- |
| | 7-yl)-7-oxo-4,5,6,7-tetrahydro-1H-pyrazolo[3,4-c]pyridine-3-carboxamide |
| 1372 | 1-(3-chlorophenyl)-N-((1,5-dimethyl-1H-pyrazol-3-yl)methyl)-6-(1,4-dimethyl-2,5- |
| | dioxo-2,3,4,5-tetrahydro-1H-benzo[e][1,4]diazepin-7-yl)-7-oxo-4,5,6,7-tetrahydro-1H- |
| | pyrazolo[3,4-c]pyridine-3-carboxamide |
| 1373 | (R)-1-(3-chlorophenyl)-6-(4-(2-hydroxyethyl)-3-methyl-5-oxo-2,3,4,5- |
| | tetrahydrobenzo[f][1,4]oxazepin-7-yl)-N-((5-methyl-1H-pyrazol-3-yl)methyl)-7-oxo- |
| | 4,5,6,7-tetrahydro-1H-pyrazolo[3,4-c]pyridine-3-carboxamide |
| 1374 | 1-(3-chlorophenyl)-N-(cyclopropylmethyl)-6-(4-ethyl-5-oxo-2,3,4,5- |
| | tetrahydrobenzo[f][1,4]oxazepin-7-yl)-7-oxo-4,5,6,7-tetrahydro-1H-pyrazolo[3,4- |
| | c]pyridine-3-carboxamide |
| 1375 | 1-(3-chlorophenyl)-6-(1,4-dimethyl-2,5-dioxo-2,3,4,5-tetrahydro-1H- |
| | benzo[e][1,4]diazepin-7-yl)-N-ethyl-7-oxo-4,5,6,7-tetrahydro-1H-pyrazolo[3,4- |
| | c]pyridine-3-carboxamide |
| 1376 | 1-(3-chlorophenyl)-6-(4-(3-hydroxypropyl)-5-oxo-2,3,4,5- |
| | tetrahydrobenzo[f][1,4]oxazepin-7-yl)-N-((4-methylpyridin-3-yl)methyl)-7-oxo-4,5,6,7- |
| | tetrahydro-1H-pyrazolo[3,4-c]pyridine-3-carboxamide |
| 1377 | 1-(3-chlorophenyl)-N-((1,5-dimethyl-1H-pyrazol-3-yl)methyl)-6-(4-(3-hydroxypropyl)- |
| | 5-oxo-2,3,4,5-tetrahydrobenzo[f][1,4]oxazepin-7-yl)-7-oxo-4,5,6,7-tetrahydro-1H- |
| | pyrazolo[3,4-c]pyridine-3-carboxamide |
| 1378 | 1-(3-chlorophenyl)-N-((5-methyl-1H-pyrazol-3-yl)methyl)-7-oxo-6-(5-oxo-4-(2,2,2- |
| | trifluoroethyl)-2,3,4,5-tetrahydrobenzo[f][1,4]oxazepin-7-yl)-4,5,6,7-tetrahydro-1H- |
| | pyrazolo[3,4-c]pyridine-3-carboxamide |
| 1379 | (S)-1-(3-chlorophenyl)-N-((1,5-dimethyl-1H-pyrazol-3-yl)methyl)-6-(4-(2- |
| | hydroxyethyl)-3-methyl-5-oxo-2,3,4,5-tetrahydrobenzo[f][1,4]oxazepin-7-yl)-7-oxo- |
| | 4,5,6,7-tetrahydro-1H-pyrazolo[3,4-c]pyridine-3-carboxamide |
| 1380 | (S)-1-(3-chlorophenyl)-6-(4-(2-hydroxyethyl)-3-methyl-5-oxo-2,3,4,5- |
| | tetrahydrobenzo[f][1,4]oxazepin-7-yl)-N-((5-methyl-1H-pyrazol-3-yl)methyl)-7-oxo- |
| | 4,5,6,7-tetrahydro-1H-pyrazolo[3,4-c]pyridine-3-carboxamide |
| 1381 | (S)-1-(3-chlorophenyl)-N-ethyl-6-(4-(2-hydroxyethyl)-3-methyl-5-oxo-2,3,4,5- |
| | tetrahydrobenzo[f][1,4]oxazepin-7-yl)-7-oxo-4,5,6,7-tetrahydro-1H-pyrazolo[3,4- |
| | c]pyridine-3-carboxamide |
| 1382 | 1-(3-chlorophenyl)-6-(4-(2,2-difluoroethyl)-5-oxo-2,3,4,5- |
| | tetrahydrobenzo[f][1,4]oxazepin-7-yl)-N-((5-methyl-1H-pyrazol-3-yl)methyl)-7-oxo- |
| | 4,5,6,7-tetrahydro-1H-pyrazolo[3,4-c]pyridine-3-carboxamide |
| 1383 | 1-(3-chlorophenyl)-N-((1,5-dimethyl-1H-pyrazol-3-yl)methyl)-6-(4-(2-(((1,5-dimethyl- |
| | 1H-pyrazol-3-yl)methyl)amino)-2-oxoethyl)-5-oxo-2,3,4,5- |
| | tetrahydrobenzo[f][1,4]oxazepin-7-yl)-7-oxo-4,5,6,7-tetrahydro-1H-pyrazolo[3,4- |
| | c]pyridine-3-carboxamide |
| 1384 | 1-(3-chlorophenyl)-N-(2-fluoroethyl)-6-(2-methyl-1-oxo-1,2,3,4-tetrahydroisoquinolin- |
| | 7-yl)-7-oxo-4,5,6,7-tetrahydro-1H-pyrazolo[3,4-c]pyridine-3-carboxamide |
| 1385 | N-((1-(3-chlorophenyl)-6-(2-methyl-1-oxo-1,2,3,4-tetrahydroisoquinolin-7-yl)-7-oxo- |
| | 4,5,6,7-tetrahydro-1H-pyrazolo[3,4-c]pyridin-3-yl)methyl)acetamide |
| 1386 | 3-(1-(3-chlorophenyl)-6-(2-methyl-1-oxo-1,2,3,4-tetrahydroisoquinolin-7-yl)-7-oxo- |
| | 4,5,6,7-tetrahydro-1H-pyrazolo[3,4-c]pyridine-3-carboxamido)propanoic acid |
| 1387 | (1-(3-chlorophenyl)-6-(2-methyl-1-oxo-1,2,3,4-tetrahydroisoquinolin-7-yl)-7-oxo- |
| | 4,5,6,7-tetrahydro-1H-pyrazolo[3,4-c]pyridine-3-carbonyl)-D-alanine |
| 1388 | 1-(3-chlorophenyl)-6-(2-methyl-1-oxo-1,2,3,4-tetrahydroisoquinolin-7-yl)-7-oxo- |
| | 4,5,6,7-tetrahydro-1H-pyrazolo[3,4-c]pyridine-3-carbonitrile |
| 1389 | 1-(3-chlorophenyl)-N-ethyl-7-oxo-6-(3-(6-oxo-5-azaspiro[2.4]heptan-5-yl)phenyl)- |
| | 4,5,6,7-tetrahydro-1H-pyrazolo[3,4-c]pyridine-3-carboxamide |
| 1390 | 1-(3-chlorophenyl)-N-ethyl-6-(3-(3-methyl-2-oxotetrahydropyrimidin-1(2H)-yl)phenyl)- |
| | 7-oxo-4,5,6,7-tetrahydro-1H-pyrazolo[3,4-c]pyridine-3-carboxamide |
| 1391 | (1-(3-chlorophenyl)-6-(2-methyl-1-oxo-1,2,3,4-tetrahydroisoquinolin-7-yl)-7-oxo- |
| | 4,5,6,7-tetrahydro-1H-pyrazolo[3,4-c]pyridine-3-carbonyl)-L-alanine |
| 1392 | (R)-1-(3-chlorophenyl)-N-ethyl-6-(4-(2-hydroxyethyl)-3-methyl-5-oxo-2,3,4,5- |
| | tetrahydrobenzo[f][1,4]oxazepin-7-yl)-7-oxo-4,5,6,7-tetrahydro-1H-pyrazolo[3,4- |
| | c]pyridine-3-carboxamide |
| 1393 | 6-(4-(2-amino-2-oxoethyl)-5-oxo-2,3,4,5-tetrahydrobenzo[f][1,4]oxazepin-7-yl)-1-(3- |
| | chlorophenyl)-N-((1,5-dimethyl-1H-pyrazol-3-yl)methyl)-7-oxo-4,5,6,7-tetrahydro-1H- |
| | pyrazolo[3,4-c]pyridine-3-carboxamide |
| 1394 | 6-(4-(2-amino-2-oxoethyl)-5-oxo-2,3,4,5-tetrahydrobenzo[f][1,4]oxazepin-7-yl)-N-((4- |
| | chloro-1,5-dimethyl-1H-pyrazol-3-yl)methyl)-1-(3-chlorophenyl)-7-oxo-4,5,6,7- |
| | tetrahydro-1H-pyrazolo[3,4-c]pyridine-3-carboxamide |
| 1395 | (S)-1-(3-chlorophenyl)-N-((1,5-dimethyl-1H-pyrazol-3-yl)methyl)-6-(3- |
| | (hydroxymethyl)-4-methyl-5-oxo-2,3,4,5-tetrahydrobenzo[f][1,4]oxazepin-7-yl)-7-oxo- |
| | 4,5,6,7-tetrahydro-1H-pyrazolo[3,4-c]pyridine-3-carboxamide |
| 1396 | (S)-1-(3-chlorophenyl)-6-(3-(hydroxymethyl)-4-methyl-5-oxo-2,3,4,5- |
| | tetrahydrobenzo[f][1,4]oxazepin-7-yl)-N-((4-methylpyridin-3-yl)methyl)-7-oxo-4,5,6,7- |
| | tetrahydro-1H-pyrazolo[3,4-c]pyridine-3-carboxamide |
| 1397 | (R)-1-(3-chlorophenyl)-N-ethyl-6-(3-(2-methyl-5-oxopyrrolidin-1-yl)phenyl)-7-oxo- |
| | 4,5,6,7-tetrahydro-1H-pyrazolo[3,4-c]pyridine-3-carboxamide |
| 1398 | (S)-1-(3-chlorophenyl)-N-ethyl-6-(3-(2-methyl-5-oxopyrrolidin-1-yl)phenyl)-7-oxo- |
| | 4,5,6,7-tetrahydro-1H-pyrazolo[3,4-c]pyridine-3-carboxamide |
| 1399 | 1-(3-chlorophenyl)-N-ethyl-6-(3-(3-methyl-2-oxo-2,3-dihydro-1H-imidazol-1- |
| | yl)phenyl)-7-oxo-4,5,6,7-tetrahydro-1H-pyrazolo[3,4-c]pyridine-3-carboxamide |
| 1400 | 1-(3-chlorophenyl)-N-ethyl-7-oxo-6-(3-(4-oxo-5-azaspiro[2.4]heptan-5-yl)phenyl)- |
| | 4,5,6,7-tetrahydro-1H-pyrazolo[3,4-c]pyridine-3-carboxamide |
| 1401 | 1-(3-chlorophenyl)-N-ethyl-6-(4-methoxy-3-(2-oxopyrrolidin-1-yl)phenyl)-7-oxo- |
| | 4,5,6,7-tetrahydro-1H-pyrazolo[3,4-c]pyridine-3-carboxamide |
| 1402 | 1-(3-chlorophenyl)-N-ethyl-6-(4-methoxy-3-(3-oxo-2-azabicyclo[3.1.0]hexan-2- |
| | yl)phenyl)-7-oxo-4,5,6,7-tetrahydro-1H-pyrazolo[3,4-c]pyridine-3-carboxamide |
| 1403 | N-((1-(3-chlorophenyl)-6-(2-methyl-1-oxo-1,2,3,4-tetrahydroisoquinolin-7-yl)-7-oxo- |
| | 4,5,6,7-tetrahydro-1H-pyrazolo[3,4-c]pyridin-3-yl)methyl)methanesulfonamide |
| 1404 | (R)-1-(3-chlorophenyl)-N-(1-cyclopropyl-2-hydroxyethyl)-6-(2-methyl-1-oxo-1,2,3,4- |
| | tetrahydroisoquinolin-7-yl)-7-oxo-4,5,6,7-tetrahydro-1H-pyrazolo[3,4-c]pyridine-3- |
| | carboxamide |
| 1405 | ethyl 1-(3-chlorophenyl)-7-oxo-6-(3-(2-oxo-4-(trifluoromethyl)pyrrolidin-1-yl)phenyl)- |
| | 4,5,6,7-tetrahydro-1H-pyrazolo[3,4-c]pyridine-3-carboxylate |
| 1406 | (S)-1-(3-chlorophenyl)-N-(1-cyclopropyl-2-hydroxyethyl)-6-(2-methyl-1-oxo-1,2,3,4- |
| | tetrahydroisoquinolin-7-yl)-7-oxo-4,5,6,7-tetrahydro-1H-pyrazolo[3,4-c]pyridine-3- |
| | carboxamide |
| 1407 | 1-(3-chlorophenyl)-N-ethyl-7-oxo-6-(3-((1R*,5R*)-3-oxo-2-azabicyclo[3.1.0]hexan-2- |
| | yl)phenyl)-4,5,6,7-tetrahydro-1H-pyrazolo[3,4-c]pyridine-3-carboxamide |
| 1408 | 1-(3-chlorophenyl)-N-ethyl-7-oxo-6-(3-((1S*,5S*)-3-oxo-2-azabicyclo[3.1.0]hexan-2- |
| | yl)phenyl)-4,5,6,7-tetrahydro-1H-pyrazolo[3,4-c]pyridine-3-carboxamide |
| 1409 | 1-(3-chlorophenyl)-N-ethyl-6-(3-(3-isopropyl-2-oxoimidazolidin-1-yl)phenyl)-7-oxo- |
| | 4,5,6,7-tetrahydro-1H-pyrazolo[3,4-c]pyridine-3-carboxamide |
| 1410 | 1-(3-chlorophenyl)-N-(2-methoxyethyl)-6-(2-methyl-1-oxo-1,2,3,4- |
| | tetrahydroisoquinolin-7-yl)-7-oxo-4,5,6,7-tetrahydro-1H-pyrazolo[3,4-c]pyridine-3- |
| | carboxamide |
| 1411 | 1-(3-chlorophenyl)-N-((3R,4S)-4-hydroxytetrahydrofuran-3-yl)-6-(2-methyl-1-oxo- |
| | 1,2,3,4-tetrahydroisoquinolin-7-yl)-7-oxo-4,5,6,7-tetrahydro-1H-pyrazolo[3,4- |
| | c]pyridine-3-carboxamide |
| 1412 | 1-(3-chlorophenyl)-N-((3R,4R)-4-hydroxytetrahydrofuran-3-yl)-6-(2-methyl-1-oxo- |
| | 1,2,3,4-tetrahydroisoquinolin-7-yl)-7-oxo-4,5,6,7-tetrahydro-1H-pyrazolo[3,4- |
| | c]pyridine-3-carboxamide |
| 1413 | 1-(3-chlorophenyl)-N-ethyl-7-oxo-6-(3-((3aR,6aR)-2-oxohexahydrocyclopenta[b]pyrrol- |
| | 1(2H)-yl)phenyl)-4,5,6,7-tetrahydro-1H-pyrazolo[3,4-c]pyridine-3-carboxamide |
| 1414 | 1-(3-chlorophenyl)-6-(2-(2-hydroxyethyl)-1-oxo-1,2,3,4-tetrahydroisoquinolin-7-yl)-N- |
| | ((6-methylpyridin-3-yl)methyl)-7-oxo-4,5,6,7-tetrahydro-1H-pyrazolo[3,4-c]pyridine-3- |
| | carboxamide |
| 1415 | 1-(3-chlorophenyl)-N-((3S,4R)-4-hydroxytetrahydrofuran-3-yl)-6-(2-methyl-1-oxo- |
| | 1,2,3,4-tetrahydroisoquinolin-7-yl)-7-oxo-4,5,6,7-tetrahydro-1H-pyrazolo[3,4- |
| | c]pyridine-3-carboxamide |
| 1416 | 1-(3-chlorophenyl)-N-((3S,4S)-4-hydroxytetrahydrofuran-3-yl)-6-(2-methyl-1-oxo- |
| | 1,2,3,4-tetrahydroisoquinolin-7-yl)-7-oxo-4,5,6,7-tetrahydro-1H-pyrazolo[3,4- |
| | c]pyridine-3-carboxamide |
| 1417 | 1-(3-chlorophenyl)-N-(3-hydroxypropyl)-6-(2-methyl-1-oxo-1,2,3,4- |
| | tetrahydroisoquinolin-7-yl)-7-oxo-4,5,6,7-tetrahydro-1H-pyrazolo[3,4-c]pyridine-3- |
| | carboxamide |
| 1418 | 1-(3-chlorophenyl)-N-ethyl-6-(4-methyl-5-oxo-2,3,4,5-tetrahydropyrido[4,3- |
| | f][1,4]oxazepin-7-yl)-7-oxo-4,5,6,7-tetrahydro-1H-pyrazolo[3,4-c]pyridine-3- |
| | carboxamide |
| 1419 | 1-(3-chlorophenyl)-N-ethyl-6-(4-methoxy-3-(piperidin-1-ylmethyl)phenyl)-7-oxo- |
| | 4,5,6,7-tetrahydro-1H-pyrazolo[3,4-c]pyridine-3-carboxamide |
| 1420 | 1-(3-chlorophenyl)-N-ethyl-6-(3-(4-methyl-2-oxopiperazin-1-yl)phenyl)-7-oxo-4,5,6,7- |
| | tetrahydro-1H-pyrazolo[3,4-c]pyridine-3-carboxamide |
| 1421 | 1-(3-chlorophenyl)-N-((1-hydroxycyclopropyl)methyl)-6-(2-methyl-1-oxo-1,2,3,4- |
| | tetrahydroisoquinolin-7-yl)-7-oxo-4,5,6,7-tetrahydro-1H-pyrazolo[3,4-c]pyridine-3- |
| | carboxamide |
| 1422 | 1-(3-chlorophenyl)-6-(3,4-dihydro-2H-benzo[b][1,4]oxazin-6-yl)-N-ethyl-7-oxo-4,5,6,7- |
| | tetrahydro-1H-pyrazolo[3,4-c]pyridine-3-carboxamide |
| 1423 | 1-(3-chlorophenyl)-N-ethyl-6-(3-(4-methylpiperazin-1-yl)phenyl)-7-oxo-4,5,6,7- |
| | tetrahydro-1H-pyrazolo[3,4-c]pyridine-3-carboxamide |
| 1424 | 1-(3-chlorophenyl)-N-(3,3-difluoropiperidin-4-yl)-6-(2-methyl-1-oxo-1,2,3,4- |
| | tetrahydroisoquinolin-7-yl)-7-oxo-4,5,6,7-tetrahydro-1H-pyrazolo[3,4-c]pyridine-3- |
| | carboxamide |
| 1425 | 1-(3-chlorophenyl)-N-(4,4-difluoropyrrolidin-3-yl)-6-(2-methyl-1-oxo-1,2,3,4- |
| | tetrahydroisoquinolin-7-yl)-7-oxo-4,5,6,7-tetrahydro-1H-pyrazolo[3,4-c]pyridine-3- |
| | carboxamide |
| 1426 | 1-(3-chlorophenyl)-N-ethyl-7-oxo-6-(3-(3-oxomorpholino)phenyl)-4,5,6,7-tetrahydro- |
| | 1H-pyrazolo[3,4-c]pyridine-3-carboxamide |
| 1427 | N-((1-aminocyclopropyl)methyl)-1-(3-chlorophenyl)-6-(2-methyl-1-oxo-1,2,3,4- |
| | tetrahydroisoquinolin-7-yl)-7-oxo-4,5,6,7-tetrahydro-1H-pyrazolo[3,4-c]pyridine-3- |
| | carboxamide |
| 1428 | 1-(3-chlorophenyl)-N-ethyl-7-oxo-6-(3-(7-oxo-2-oxa-6-azaspiro[3.4]octan-6-yl)phenyl)- |
| | 4,5,6,7-tetrahydro-1H-pyrazolo[3,4-c]pyridine-3-carboxamide |
| 1429 | 6-(1′-acetylspiro[cyclopropane-1,3′-indolin]-6′-yl)-1-(3-chlorophenyl)-N-ethyl-7-oxo- |
| | 4,5,6,7-tetrahydro-1H-pyrazolo[3,4-c]pyridine-3-carboxamide |
| 1430 | 6-(3-(5-azaspiro[2.4]heptan-5-yl)phenyl)-1-(3-chlorophenyl)-N-ethyl-7-oxo-4,5,6,7- |
| | tetrahydro-1H-pyrazolo[3,4-c]pyridine-3-carboxamide |
| 1431 | 6-(3-(3-azabicyclo[3.1.0]hexan-3-yl)phenyl)-1-(3-chlorophenyl)-N-ethyl-7-oxo-4,5,6,7- |
| | tetrahydro-1H-pyrazolo[3,4-c]pyridine-3-carboxamide |
| 1432 | 1-(3-chlorophenyl)-N-ethyl-6-(3-(4-methyl-3-oxopiperazin-1-yl)phenyl)-7-oxo-4,5,6,7- |
| | tetrahydro-1H-pyrazolo[3,4-c]pyridine-3-carboxamide |
| 1433 | 1-(3-chlorophenyl)-N-ethyl-6-(2-fluoro-5-(3-oxo-2-azabicyclo[3.1.0]hexan-2- |
| | yl)phenyl)-7-oxo-4,5,6,7-tetrahydro-1H-pyrazolo[3,4-c]pyridine-3-carboxamide |
| 1434 | 1-(3-chlorophenyl)-N-ethyl-6-(2-methyl-1-oxo-2,3,4,5-tetrahydro-1H-3,5- |
| | methanobenzo[c]azepin-8-yl)-7-oxo-4,5,6,7-tetrahydro-1H-pyrazolo[3,4-c]pyridine-3- |
| | carboxamide |
| 1435 | 1-(3-chlorophenyl)-N-ethyl-6-(8-fluoro-4-methyl-5-oxo-2,3,4,5- |
| | tetrahydrobenzo[f][1,4]oxazepin-7-yl)-7-oxo-4,5,6,7-tetrahydro-1H-pyrazolo[3,4- |
| | c]pyridine-3-carboxamide |
| 1436 | 1-(3-chlorophenyl)-N-ethyl-6-(3-(3-methyl-3,8-diazabicyclo[3.2.1]octan-8-yl)phenyl)- |
| | 7-oxo-4,5,6,7-tetrahydro-1H-pyrazolo[3,4-c]pyridine-3-carboxamide |
| 1437 | 1-(3-chlorophenyl)-N-(3-hydroxybicyclo[1.1.1]pentan-1-yl)-6-(2-methyl-1,2,3,4- |
| | tetrahydroisoquinolin-7-yl)-7-oxo-4,5,6,7-tetrahydro-1H-pyrazolo[3,4-c]pyridine-3- |
| | carboxamide |
| 1438 | 1-(3-chlorophenyl)-6-(2-methyl-1-oxo-1,2,3,4-tetrahydroisoquinolin-7-yl)-7-oxo-N-(5- |
| | oxaspiro[2.4]heptan-7-yl)-4,5,6,7-tetrahydro-1H-pyrazolo[3,4-c]pyridine-3- |
| | carboxamide |
| 2001 | (S)-1-(3-chloro-2-fluorophenyl)-N-(2-hydroxy-1-phenylethyl)-6-(2-methyl-1,2,3,4- |
| | tetrahydroisoquinolin-7-yl)-7-oxo-4,5,6,7-tetrahydro-1H-pyrazolo[3,4-c]pyridine-3- |
| | carboxamide |
| 2002 | 1-(3-chloro-2-fluorophenyl)-6-(2-(2-hydroxyethyl)-1,2,3,4-tetrahydroisoquinolin-7-yl)- |
| | N-((4-methylpyridin-3-yl)methyl)-7-oxo-4,5,6,7-tetrahydro-1H-pyrazolo[3,4- |
| | c]pyridine-3-carboxamide |
| 2003 | 6-(2-(2-hydroxyethyl)-1,2,3,4-tetrahydroisoquinolin-7-yl)-1-(4-methoxyphenyl)-N-((4- |
| | methylpyridin-3-yl)methyl)-7-oxo-4,5,6,7-tetrahydro-1H-pyrazolo[3,4-c]pyridine-3- |
| | carboxamide |
| 2004 | 1-(3-chloro-2-fluorophenyl)-N-(cyclopropylmethyl)-6-(2-methyl-1-oxo-1,2,3,4- |
| | tetrahydroisoquinolin-7-yl)-7-oxo-4,5,6,7-tetrahydro-1H-pyrazolo[3,4-c]pyridine-3- |
| | carboxamide |
| 2005 | 1-(3-cyanophenyl)-N-(cyclopropylmethyl)-6-(2-methyl-1-oxo-1,2,3,4- |
| | tetrahydroisoquinolin-7-yl)-7-oxo-4,5,6,7-tetrahydro-1H-pyrazolo[3,4-c]pyridine-3- |
| | carboxamide |
| 2006 | 1-(3-carbamoylphenyl)-N-(cyclopropylmethyl)-6-(2-methyl-1-oxo-1,2,3,4- |
| | tetrahydroisoquinolin-7-yl)-7-oxo-4,5,6,7-tetrahydro-1H-pyrazolo[3,4-c]pyridine-3- |
| | carboxamide |
| 2007 | 1-(3-chloro-4-methylphenyl)-6-(2-(2-hydroxyethyl)-1,2,3,4-tetrahydroisoquinolin-7- |
| | yl)-N-((4-methylpyridin-3-yl)methyl)-7-oxo-4,5,6,7-tetrahydro-1H-pyrazolo[3,4- |
| | c]pyridine-3-carboxamide |
| 2008 | 6-(2-(2-hydroxyethyl)-1,2,3,4-tetrahydroisoquinolin-7-yl)-N-((4-methylpyridin-3- |
| | yl)methyl)-7-oxo-1-(m-tolyl)-4,5,6,7-tetrahydro-1H-pyrazolo[3,4-c]pyridine-3- |
| | carboxamide |
| 2009 | 6-(2-(2-hydroxyethyl)-1,2,3,4-tetrahydroisoquinolin-7-yl)-N-((4-methylpyridin-3- |
| | yl)methyl)-7-oxo-1-(3-(trifluoromethyl)phenyl)-4,5,6,7-tetrahydro-1H-pyrazolo[3,4- |
| | c]pyridine-3-carboxamide |
| 2010 | 1-(3-cyanophenyl)-6-(2-(2-hydroxyethyl)-1,2,3,4-tetrahydroisoquinolin-7-yl)-N-((4- |
| | methylpyridin-3-yl)methyl)-7-oxo-4,5,6,7-tetrahydro-1H-pyrazolo[3,4-c]pyridine-3- |
| | carboxamide |
| 2011 | N-(cyclopropylmethyl)-6-(2-methyl-1-oxo-1,2,3,4-tetrahydroisoquinolin-7-yl)-7-oxo-1- |
| | (m-tolyl)-4,5,6,7-tetrahydro-1H-pyrazolo[3,4-c]pyridine-3-carboxamide |
| 2012 | 1-(3-cyanophenyl)-N-((1,5-dimethyl-1H-pyrazol-3-yl)methyl)-6-(2-methyl-1-oxo- |
| | 1,2,3,4-tetrahydroisoquinolin-7-yl)-7-oxo-4,5,6,7-tetrahydro-1H-pyrazolo[3,4- |
| | c]pyridine-3-carboxamide |
| 2013 | 1-(3-chloro-5-cyanophenyl)-N-(cyclopropylmethyl)-6-(2-methyl-1-oxo-1,2,3,4- |
| | tetrahydroisoquinolin-7-yl)-7-oxo-4,5,6,7-tetrahydro-1H-pyrazolo[3,4-c]pyridine-3- |
| | carboxamide |
| 2014 | 1-(3-carbamoyl-5-chlorophenyl)-N-(cyclopropylmethyl)-6-(2-methyl-1-oxo-1,2,3,4- |
| | tetrahydroisoquinolin-7-yl)-7-oxo-4,5,6,7-tetrahydro-1H-pyrazolo[3,4-c]pyridine-3- |
| | carboxamide |
| 2015 | 1-(5-chlorothiophen-3-yl)-N-(cyclopropylmethyl)-6-(2-methyl-1-oxo-1,2,3,4- |
| | tetrahydroisoquinolin-7-yl)-7-oxo-4,5,6,7-tetrahydro-1H-pyrazolo[3,4-c]pyridine-3- |
| | carboxamide |
| 2016 | 1-(4-chloropyridin-2-yl)-N-(cyclopropylmethyl)-6-(2-methyl-1-oxo-1,2,3,4- |
| | tetrahydroisoquinolin-7-yl)-7-oxo-4,5,6,7-tetrahydro-1H-pyrazolo[3,4-c]pyridine-3- |
| | carboxamide |
| 2017 | 1-(3-(aminomethyl)-5-chlorophenyl)-N-((1,5-dimethyl-1H-pyrazol-3-yl)methyl)-6-(2- |
| | methyl-1-oxo-1,2,3,4-tetrahydroisoquinolin-7-yl)-7-oxo-4,5,6,7-tetrahydro-1H- |
| | pyrazolo[3,4-c]pyridine-3-carboxamide |
| 2018 | 1-(3-(aminomethyl)-5-chlorophenyl)-N-(cyclopropylmethyl)-6-(2-methyl-1-oxo- |
| | 1,2,3,4-tetrahydroisoquinolin-7-yl)-7-oxo-4,5,6,7-tetrahydro-1H-pyrazolo[3,4- |
| | c]pyridine-3-carboxamide |
| 2019 | 1-(3-(aminomethyl)phenyl)-N-(cyclopropylmethyl)-6-(2-methyl-1-oxo-1,2,3,4- |
| | tetrahydroisoquinolin-7-yl)-7-oxo-4,5,6,7-tetrahydro-1H-pyrazolo[3,4-c]pyridine-3- |
| | carboxamide |
| 2020 | 1-(6-chloropyridin-2-yl)-N-(cyclopropylmethyl)-6-(2-methyl-1-oxo-1,2,3,4- |
| | tetrahydroisoquinolin-7-yl)-7-oxo-4,5,6,7-tetrahydro-1H-pyrazolo[3,4-c]pyridine-3- |
| | carboxamide |
| 2021 | 1-(5-chloropyridin-3-yl)-N-(cyclopropylmethyl)-6-(2-methyl-1-oxo-1,2,3,4- |
| | tetrahydroisoquinolin-7-yl)-7-oxo-4,5,6,7-tetrahydro-1H-pyrazolo[3,4-c]pyridine-3- |
| | carboxamide |
| 2022 | ethyl 1-(3-(aminomethyl)-5-methylphenyl)-6-(2-methyl-1-oxo-1,2,3,4- |
| | tetrahydroisoquinolin-7-yl)-7-oxo-4,5,6,7-tetrahydro-1H-pyrazolo[3,4-c]pyridine-3- |
| | carboxylate |
| 2023 | ethyl 6-(2-methyl-1-oxo-1,2,3,4-tetrahydroisoquinolin-7-yl)-1-(6-methylpyridin-2-yl)- |
| | 7-oxo-4,5,6,7-tetrahydro-1H-pyrazolo[3,4-c]pyridine-3-carboxylate |
| 2024 | 1-(3-(aminomethyl)-5-methylphenyl)-N-ethyl-6-(2-methyl-1-oxo-1,2,3,4- |
| | tetrahydroisoquinolin-7-yl)-7-oxo-4,5,6,7-tetrahydro-1H-pyrazolo[3,4-c]pyridine-3- |
| | carboxamide |
| 2025 | 1-(3-(aminomethyl)-5-methylphenyl)-6-(2-methyl-1-oxo-1,2,3,4- |
| | tetrahydroisoquinolin-7-yl)-7-oxo-4,5,6,7-tetrahydro-1H-pyrazolo[3,4-c]pyridine-3- |
| | carboxylic acid |
| 2027 | N-ethyl-6-(2-methyl-1-oxo-1,2,3,4-tetrahydroisoquinolin-7-yl)-1-(5-methylpyridin-3- |
| | yl)-7-oxo-4,5,6,7-tetrahydro-1H-pyrazolo[3,4-c]pyridine-3-carboxamide |
| 2028 | N-ethyl-6-(2-methyl-1-oxo-1,2,3,4-tetrahydroisoquinolin-7-yl)-1-(5-methylpyridin-2- |
| | yl)-7-oxo-4,5,6,7-tetrahydro-1H-pyrazolo[3,4-c]pyridine-3-carboxamide |
| 2029 | 1-(5-chloro-2-methylphenyl)-N-ethyl-6-(2-methyl-1-oxo-1,2,3,4-tetrahydroisoquinolin- |
| | 7-yl)-7-oxo-4,5,6,7-tetrahydro-1H-pyrazolo[3,4-c]pyridine-3-carboxamide |
| 2030 | 1-(5-chloro-2-fluorophenyl)-N-ethyl-6-(2-methyl-1-oxo-1,2,3,4-tetrahydroisoquinolin- |
| | 7-yl)-7-oxo-4,5,6,7-tetrahydro-1H-pyrazolo[3,4-c]pyridine-3-carboxamide |
| 2031 | 1-(6-amino-5-chloropyridin-3-yl)-N-ethyl-6-(2-methyl-1-oxo-1,2,3,4- |
| | tetrahydroisoquinolin-7-yl)-7-oxo-4,5,6,7-tetrahydro-1H-pyrazolo[3,4-c]pyridine-3- |
| | carboxamide |
| 2032 | N-ethyl-1-(2-fluoro-3-methylphenyl)-6-(2-methyl-1-oxo-1,2,3,4-tetrahydroisoquinolin- |
| | 7-yl)-7-oxo-4,5,6,7-tetrahydro-1H-pyrazolo[3,4-c]pyridine-3-carboxamide |
| 2033 | 1-(3-chloro-5-(hydroxymethyl)phenyl)-N-ethyl-6-(2-methyl-1-oxo-1,2,3,4- |
| | tetrahydroisoquinolin-7-yl)-7-oxo-4,5,6,7-tetrahydro-1H-pyrazolo[3,4-c]pyridine-3- |
| | carboxamide |
| 2034 | N-ethyl-1-(2-fluoro-4-methylphenyl)-6-(2-methyl-1-oxo-1,2,3,4-tetrahydroisoquinolin- |
| | 7-yl)-7-oxo-4,5,6,7-tetrahydro-1H-pyrazolo[3,4-c]pyridine-3-carboxamide |
| 2035 | N-ethyl-6-(2-methyl-1-oxo-1,2,3,4-tetrahydroisoquinolin-7-yl)-1-(4-methylpyridin-2- |
| | yl)-7-oxo-4,5,6,7-tetrahydro-1H-pyrazolo[3,4-c]pyridine-3-carboxamide |
| 2036 | 1-(3-chloro-2-methylphenyl)-N-ethyl-6-(2-methyl-1-oxo-1,2,3,4-tetrahydroisoquinolin- |
| | 7-yl)-7-oxo-4,5,6,7-tetrahydro-1H-pyrazolo[3,4-c]pyridine-3-carboxamide |
| 2037 | N-ethyl-1-(6-methoxypyridin-2-yl)-6-(2-methyl-1-oxo-1,2,3,4-tetrahydroisoquinolin-7- |
| | yl)-7-oxo-4,5,6,7-tetrahydro-1H-pyrazolo[3,4-c]pyridine-3-carboxamide |
| 2038 | 3-(3-(methoxymethyl)-6-(2-methyl-1-oxo-1,2,3,4-tetrahydroisoquinolin-7-yl)-7-oxo- |
| | 4,5,6,7-tetrahydro-1H-pyrazolo[3,4-c]pyridin-1-yl)-2-methylbenzonitrile |
| 2039 | (R)-1-(3-chloro-2-fluorophenyl)-6-(2-methyl-1-oxo-1,2,3,4-tetrahydroisoquinolin-7-yl)- |
| | 7-oxo-N-(tetrahydrofuran-3-yl)-4,5,6,7-tetrahydro-1H-pyrazolo[3,4-c]pyridine-3- |
| | carboxamide |
| 2040 | 1-(3-chloro-2-fluorophenyl)-6-(2-methyl-1-oxo-1,2,3,4-tetrahydroisoquinolin-7-yl)-N- |
| | ((5-methyl-1H-pyrazol-3-yl)methyl)-7-oxo-4,5,6,7-tetrahydro-1H-pyrazolo[3,4- |
| | c]pyridine-3-carboxamide |
| 2041 | 7-(1-(3-(aminomethyl)-5-chlorophenyl)-3-(methoxymethyl)-7-oxo-1,4,5,7-tetrahydro- |
| | 6H-pyrazolo[3,4-c]pyridin-6-yl)-2-methyl-3,4-dihydroisoquinolin-1(2H)-one |
| 2042 | 1-(3-cyano-2-methylphenyl)-N-ethyl-6-(2-methyl-1-oxo-1,2,3,4-tetrahydroisoquinolin- |
| | 7-yl)-7-oxo-4,5,6,7-tetrahydro-1H-pyrazolo[3,4-c]pyridine-3-carboxamide |
| 2043 | (R)-1-(3-chloro-2-fluorophenyl)-N-(1-cyclopropyl-2-hydroxyethyl)-6-(2-methyl-1-oxo- |
| | 1,2,3,4-tetrahydroisoquinolin-7-yl)-7-oxo-4,5,6,7-tetrahydro-1H-pyrazolo[3,4- |
| | c]pyridine-3-carboxamide |
| 2044 | 1-(3-chloro-2-fluorophenyl)-6-(2-methyl-1-oxo-1,2,3,4-tetrahydroisoquinolin-7-yl)-N- |
| | ((6-methylpyridin-3-yl)methyl)-7-oxo-4,5,6,7-tetrahydro-1H-pyrazolo[3,4-c]pyridine-3- |
| | carboxamide |
| 2045 | N-ethyl-6-(2-methyl-1-oxo-1,2,3,4-tetrahydroisoquinolin-7-yl)-1-(4-methylthiophen-2- |
| | yl)-7-oxo-4,5,6,7-tetrahydro-1H-pyrazolo[3,4-c]pyridine-3-carboxamide |
| 2046 | 1-(3-chloro-2-fluorophenyl)-N-(cyclopropylmethyl)-6-(4-methyl-5-oxo-2,3,4,5- |
| | tetrahydrobenzo[f][1,4]oxazepin-7-yl)-7-oxo-4,5,6,7-tetrahydro-1H-pyrazolo[3,4- |
| | c]pyridine-3-carboxamide |
| 2047 | 7-(1-(3-(aminomethyl)-5-chlorophenyl)-7-oxo-1,4,5,7-tetrahydro-6H-pyrazolo[3,4- |
| | c]pyridin-6-yl)-2-methyl-3,4-dihydroisoquinolin-1(2H)-one |
| 2048 | (R)-1-(3-chloro-2-fluorophenyl)-6-(4-methyl-5-oxo-2,3,4,5- |
| | tetrahydrobenzo[f][1,4]oxazepin-7-yl)-7-oxo-N-(tetrahydrofuran-3-yl)-4,5,6,7- |
| | tetrahydro-1H-pyrazolo[3,4-c]pyridine-3-carboxamide |
| 2049 | (R)-1-(3-chloro-2-fluorophenyl)-N-(1-cyclopropyl-2-hydroxyethyl)-6-(4-methyl-5-oxo- |
| | 2,3,4,5-tetrahydrobenzo[f][1,4]oxazepin-7-yl)-7-oxo-4,5,6,7-tetrahydro-1H- |
| | pyrazolo[3,4-c]pyridine-3-carboxamide |
| 2050 | 1-(3-chloro-2-fluorophenyl)-N-(3-hydroxybicyclo[1.1.1]pentan-1-yl)-6-(4-methyl-5- |
| | oxo-2,3,4,5-tetrahydrobenzo[f][1,4]oxazepin-7-yl)-7-oxo-4,5,6,7-tetrahydro-1H- |
| | pyrazolo[3,4-c]pyridine-3-carboxamide |
| 2051 | 1-(3-chloro-5-methylphenyl)-N-ethyl-6-(2-methyl-1-oxo-1,2,3,4-tetrahydroisoquinolin- |
| | 7-yl)-7-oxo-4,5,6,7-tetrahydro-1H-pyrazolo[3,4-c]pyridine-3-carboxamide |
| 2052 | 1-(3-chloro-2-(hydroxymethyl)phenyl)-N-ethyl-6-(2-methyl-1-oxo-1,2,3,4- |
| | tetrahydroisoquinolin-7-yl)-7-oxo-4,5,6,7-tetrahydro-1H-pyrazolo[3,4-c]pyridine-3- |
| | carboxamide |
| 2053 | (S)-1-(3-chloro-2-fluorophenyl)-N-(1-cyclopropyl-2-hydroxyethyl)-6-(4-methyl-5-oxo- |
| | 2,3,4,5-tetrahydrobenzo[f][1,4]oxazepin-7-yl)-7-oxo-4,5,6,7-tetrahydro-1H- |
| | pyrazolo[3,4-c]pyridine-3-carboxamide |
| 2054 | 1-(3-chloro-4-hydroxyphenyl)-N-ethyl-6-(2-methyl-1-oxo-1,2,3,4- |
| | tetrahydroisoquinolin-7-yl)-7-oxo-4,5,6,7-tetrahydro-1H-pyrazolo[3,4-c]pyridine-3- |
| | carboxamide |
| 2056 | (1-(3-(aminomethyl)-5-chlorophenyl)-6-(2-methyl-1-oxo-1,2,3,4- |
| | tetrahydroisoquinolin-7-yl)-7-oxo-4,5,6,7-tetrahydro-1H-pyrazolo[3,4-c]pyridine-3- |
| | carbonyl)glycine |
| 2057 | 1-(4-chloro-3-methylphenyl)-N-ethyl-6-(2-methyl-1-oxo-1,2,3,4-tetrahydroisoquinolin- |
| | 7-yl)-7-oxo-4,5,6,7-tetrahydro-1H-pyrazolo[3,4-c]pyridine-3-carboxamide |
| 2058 | 1-(3-chloro-5-(methoxymethyl)phenyl)-N-ethyl-6-(2-methyl-1-oxo-1,2,3,4- |
| | tetrahydroisoquinolin-7-yl)-7-oxo-4,5,6,7-tetrahydro-1H-pyrazolo[3,4-c]pyridine-3- |
| | carboxamide |
| 3001 | 1-(3-chlorophenyl)-6-(2-methyl-1,2,3,4-tetrahydroisoquinolin-7-yl)-7-oxo-6,7-dihydro- |
| | 1H-pyrazolo[4,3-d]pyrimidine-3-carbonitrile |
| 3002 | 1-(3-chlorophenyl)-5-methyl-6-(2-methyl-1,2,3,4-tetrahydroisoquinolin-7-yl)-7-oxo- |
| | 6,7-dihydro-1H-pyrazolo[4,3-d]pyrimidine-3-carbonitrile |
| 3003 | 1-(3-chlorophenyl)-N-(cyclopropylmethyl)-6-(2-(2-hydroxyethyl)-1,2,3,4- |
| | tetrahydroisoquinolin-7-yl)-5-methyl-7-oxo-6,7-dihydro-1H-pyrazolo[4,3-d]pyrimidine- |
| | 3-carboxamide |
| 3004 | 1-(3-chlorophenyl)-N-(cyclopropylmethyl)-5-methyl-6-(2-methyl-1,2,3,4- |
| | tetrahydroisoquinolin-7-yl)-7-oxo-6,7-dihydro-1H-pyrazolo[4,3-d]pyrimidine-3- |
| | carboxamide |
| 3005 | 1-(3-chlorophenyl)-N-((1,4-dimethyl-1H-pyrazol-3-yl)methyl)-6-(2-(2-hydroxyethyl)- |
| | 1,2,3,4-tetrahydroisoquinolin-7-yl)-5-methyl-7-oxo-6,7-dihydro-1H-pyrazolo[4,3- |
| | d]pyrimidine-3-carboxamide |
| 3006 | 1-(3-chlorophenyl)-N-((1,4-dimethyl-1H-pyrazol-3-yl)methyl)-5-methyl-6-(2-methyl- |
| | 1,2,3,4-tetrahydroisoquinolin-7-yl)-7-oxo-6,7-dihydro-1H-pyrazolo[4,3-d]pyrimidine-3- |
| | carboxamide |
| 3007 | N-((1-(3-chlorophenyl)-6-(2-(2-hydroxyethyl)-1,2,3,4-tetrahydroisoquinolin-7-yl)-5- |
| | methyl-7-oxo-6,7-dihydro-1H-pyrazolo[4,3-d]pyrimidin-3- |
| | yl)methyl)cyclopropanesulfonamide |
| 3008 | N-((1-(3-chlorophenyl)-6-(2-(2-hydroxyethyl)-1,2,3,4-tetrahydroisoquinolin-7-yl)-5- |
| | methyl-7-oxo-6,7-dihydro-1H-pyrazolo[4,3-d]pyrimidin-3- |
| | yl)methyl)cyclopropanecarboxamide |
| 3009 | N-((1-(3-chlorophenyl)-6-(2-(2-hydroxyethyl)-1,2,3,4-tetrahydroisoquinolin-7-yl)-5- |
| | methyl-7-oxo-6,7-dihydro-1H-pyrazolo[4,3-d]pyrimidin-3-yl)methyl)-1,4-dimethyl-1H- |
| | pyrazole-3-carboxamide |
| 3010 | N-((1-(3-chlorophenyl)-6-(2-(2-hydroxyethyl)-1,2,3,4-tetrahydroisoquinolin-7-yl)-5- |
| | methyl-7-oxo-6,7-dihydro-1H-pyrazolo[4,3-d]pyrimidin-3-yl)methyl)-4- |
| | methylnicotinamide |
| 3011 | 1-(3-chlorophenyl)-6-(2-(2-hydroxyethyl)-1,2,3,4-tetrahydroisoquinolin-7-yl)-5- |
| | methyl-N-((4-methylpyridin-3-yl)methyl)-7-oxo-6,7-dihydro-1H-pyrazolo[4,3- |
| | d]pyrimidine-3-carboxamide |
| 3012 | 1-(3-chlorophenyl)-6-(2-(2-hydroxyethyl)-1,2,3,4-tetrahydroisoquinolin-7-yl)-5- |
| | methyl-7-oxo-6,7-dihydro-1H-pyrazolo[4,3-d]pyrimidine-3-carbonitrile |
| 3013 | 1-(3-chlorophenyl)-6-(2-(2-hydroxyethyl)-1,2,3,4-tetrahydroisoquinolin-7-yl)-7-oxo- |
| | 6,7-dihydro-1H-pyrazolo[4,3-d]pyrimidine-3-carbonitrile |
| 3014 | 1-(3-chlorophenyl)-N-(cyclopropylmethyl)-5-methyl-6-(2-methyl-1-oxo-1,2,3,4- |
| | tetrahydroisoquinolin-7-yl)-7-oxo-6,7-dihydro-1H-pyrazolo[4,3-d]pyrimidine-3- |
| | carboxamide |
| 3015 | 1-(3-chlorophenyl)-N-((1,5-dimethyl-1H-pyrazol-3-yl)methyl)-6-(2-(2-hydroxyethyl)-1- |
| | oxo-1,2,3,4-tetrahydroisoquinolin-7-yl)-5-methyl-7-oxo-6,7-dihydro-1H-pyrazolo[4,3- |
| | d]pyrimidine-3-carboxamide |
| 3016 | 1-(3-chlorophenyl)-N-(cyclopropylmethyl)-6-(2-(2-hydroxyethyl)-1-oxo-1,2,3,4- |
| | tetrahydroisoquinolin-7-yl)-5-methyl-7-oxo-6,7-dihydro-1H-pyrazolo[4,3-d]pyrimidine- |
| | 3-carboxamide |
| 3017 | 1-(3-chlorophenyl)-6-(2-(2-hydroxyethyl)-1-oxo-1,2,3,4-tetrahydroisoquinolin-7-yl)-5- |
| | methyl-N-((4-methylpyridin-3-yl)methyl)-7-oxo-6,7-dihydro-1H-pyrazolo[4,3- |
| | d]pyrimidine-3-carboxamide |
| 3018 | 1-(3-chlorophenyl)-6-(2-(2-hydroxyethyl)-1-oxo-1,2,3,4-tetrahydroisoquinolin-7-yl)-5- |
| | methyl-7-oxo-6,7-dihydro-1H-pyrazolo[4,3-d]pyrimidine-3-carboxylic acid |
| 3019 | ethyl 1-(3-chlorophenyl)-6-(2-(2-hydroxyethyl)-1-oxo-1,2,3,4-tetrahydroisoquinolin-7- |
| | yl)-5-methyl-7-oxo-6,7-dihydro-1H-pyrazolo[4,3-d]pyrimidine-3-carboxylate |
| 3020 | 1-(3-chlorophenyl)-5-methyl-6-(2-methyl-1-oxo-1,2,3,4-tetrahydroisoquinolin-7-yl)-N- |
| | ((5-methyl-1H-pyrazol-3-yl)methyl)-7-oxo-6,7-dihydro-1H-pyrazolo[4,3-d]pyrimidine- |
| | 3-carboxamide |
| 3021 | 1-(3-chlorophenyl)-N-((1,5-dimethyl-1H-pyrazol-3-yl)methyl)-5-methyl-6-(2-methyl-1- |
| | oxo-1,2,3,4-tetrahydroisoquinolin-7-yl)-7-oxo-6,7-dihydro-1H-pyrazolo[4,3- |
| | d]pyrimidine-3-carboxamide |
| 3022 | 1-(3-chlorophenyl)-N-ethyl-5-methyl-6-(2-methyl-1-oxo-1,2,3,4-tetrahydroisoquinolin- |
| | 7-yl)-7-oxo-6,7-dihydro-1H-pyrazolo[4,3-d]pyrimidine-3-carboxamide |
| 3023 | 1-(3-chloro-2-fluorophenyl)-N-(cyclopropylmethyl)-5-methyl-6-(4-methyl-5-oxo- |
| | 2,3,4,5-tetrahydrobenzo[f][1,4]oxazepin-7-yl)-7-oxo-6,7-dihydro-1H-pyrazolo[4,3- |
| | d]pyrimidine-3-carboxamide |
| 4001 | 1-(3-chlorophenyl)-N-((1,5-dimethyl-1H-pyrazol-3-yl)methyl)-5-(2-(2-hydroxyethyl)- |
| | 1,2,3,4-tetrahydroisoquinolin-7-yl)-6-oxo-1,4,5,6-tetrahydropyrrolo[3,4-c]pyrazole-3- |
| | carboxamide |
| 4002 | 2-(3-(3-chlorophenyl)-5-(2-(2-hydroxyethyl)-1,2,3,4-tetrahydroisoquinolin-7-yl)-4-oxo- |
| | 4,5,6,7-tetrahydro-1H-pyrazolo[4,3-c]pyridin-1-yl)-N-(cyclopropylmethyl)acetamide |
| 4003 | 3-(3-chlorophenyl)-5-(2-(2-hydroxyethyl)-1,2,3,4-tetrahydroisoquinolin-7-yl)-N-((4- |
| | methylpyridin-3-yl)methyl)-4-oxo-4,5,6,7-tetrahydro-1H-pyrazolo[4,3-c]pyridine-1- |
| | carboxamide |
| 4004 | 2-(3-(3-chlorophenyl)-5-(2-(2-hydroxyethyl)-1,2,3,4-tetrahydroisoquinolin-7-yl)-4-oxo- |
| | 4,5,6,7-tetrahydro-1H-pyrazolo[4,3-c]pyridin-1-yl)-N-cyclopropylacetamide |

TABLE 6
¹H NMR data of examples (solvent DMSO-d6 unless otherwise indicated)

| Example | |
|---|---|
| Number | NMR write-up |
| 1001 | 1.32-1.42 (2H, m), 1.44-1.54 (4H, m), 2.21-2.38 (4H, m), 3.21 (4H, t, J = 6.6 Hz), 4.06 |
| | (2H, t, J = 6.6 Hz), 4.45 (2H, d, J = 6.3 Hz), 7.13-7.21 (1H, m), 7.21-7.28 (3H, m), 7.29- |
| | 7.37 (5H, m), 7.47-7.55 (2H, m), 7.64 (1H, dt, J = 6.8, 2.1 Hz), 7.75-7.83 (1H, m), 8.25 |
| | (1H, s), 9.02 (1H, t, J = 6.3 Hz) |
| 1002 | 0.23-0.27 (2H, m), 0.40-0.46 (2H, m), 1.02-1.09 (1H, m), 1.15-1.24 (1H, m), 1.39 (2H, |
| | d, J = 6.9 Hz), 1.48 (4H, q, J = 5.5 Hz), 2.28-2.35 (4H, m), 3.13 (2H, t, J = 6.5 Hz), 3.22 (3H, |
| | d, J = 6.6 Hz), 4.07 (2H, td, J = 6.7, 2.2 Hz), 6.77 (1H, s), 7.18 (1H, dt, J = 7.5, 1.4 Hz), 7.22- |
| | 7.28 (2H, m), 7.34 (1H, t, J = 7.7 Hz), 7.49-7.56 (2H, m), 7.64 (1H, dt, J = 6.7, 2.1 Hz), 7.80 |
| | (1H, t, J = 2.1 Hz), 8.43 (1H, s), 8.51 (1H, t, J = 6.0 Hz) |
| 1003 | 1.32-1.42 (2H, m), 1.41-1.52 (4H, m), 2.23-2.36 (4H, m), 3.20 (4H, t, J = 6.6 Hz), 4.06 |
| | (2H, t, J = 6.6 Hz), 4.46 (2H, d, J = 6.2 Hz), 6.84 (1H, s), 7.17 (1H, d, J = 7.5 Hz), 7.20-7.29 |
| | (2H, m), 7.28-7.40 (2H, m), 7.47-7.56 (2H, m), 7.63 (1H, dt, J = 7.1, 2.0 Hz), 7.74 (1H, dt, |
| | J = 7.9, 2.1 Hz), 7.77-7.81 (1H, m), 8.38 (1H, s), 8.46 (1H, dd, J = 4.8, 1.7 Hz), 8.56 (1H, d, |
| | J = 2.2 Hz), 9.14 (1H, t, J = 6.3 Hz) |
| 1004 | 1.31-1.41 (2H, m), 1.43-1.55 (4H, m), 2.22-2.38 (4H, m), 2.93 (2H, s), 3.06-3.15 (2H, |
| | m), 3.30 (3H, s), 4.07 (2H, q, J = 6.6 Hz), 4.72 (1H, s), 4.99 (1H, s), 7.17 (1H, d, J = 7.6 Hz), |
| | 7.21-7.39 (7H, m), 7.39-7.63 (4H, m), 7.61-7.78 (1H, m) |
| 1005 | 2.33 (3H, s), 2.59 (2H, t, J = 5.9 Hz), 2.81 (2H, t, J = 5.9 Hz), 3.20 (4H, t, J = 6.6 Hz), 4.03 |
| | (2H, t, J = 6.6 Hz), 4.45 (2H, d, J = 6.2 Hz), 6.74 (1H, s), 7.04 (1H, s), 7.12 (2H, d, J = 2.1 Hz), |
| | 7.25 (1H, td, J = 5.5, 2.8 Hz), 7.30-7.37 (4H, m), 7.48-7.56 (2H, m), 7.63 (1H, dt, J = 6.8, |
| | 2.1 Hz), 7.79 (1H, d, J = 2.1 Hz), 8.38 (1H, s), 9.03 (1H, t, J = 6.3 Hz) |
| 1006 | 1.31-1.42 (2H, m), 1.44-1.52 (4H, m), 2.21-2.38 (4H, m), 3.17-3.23 (2H, m), 3.40 (2H, |
| | s), 4.08 (2H, t, J = 6.6 Hz), 5.39 (2H, s), 7.13-7.20 (1H, m), 7.21-7.28 (2H, m), 7.30-7.35 |
| | (1H, m), 7.35-7.39 (1H, m), 7.39-7.44 (2H, m), 7.46-7.50 (2H, m), 7.50-7.61 (3H, m), |
| | 7.75 (1H, t, J = 2.0 Hz) |
| 1007 | (CDCl3) 1.44 (5H, t, J = 7.1 Hz), 1.57 (4H, s), 2.37 (4H, s), 3.32 (2H, t, J = 6.7 Hz), 3.46 (2H, |
| | s), 4.14 (2H, t, J = 6.7 Hz), 4.47 (2H, q, J = 7.1 Hz), 7.21 (2H, d, J = 7.6 Hz), 7.29-7.39 (4H, |
| | m), 7.48 (1H, dt, J = 7.6, 1.7 Hz), 7.62 (1H, t, J = 2.0 Hz) |
| 1008 | 1.31-1.41 (2H, m), 1.43-1.53 (4H, m), 2.21-2.38 (4H, m), 3.13-3.21 (2H, m), 3.40 (2H, |
| | s), 3.62-3.81 (2H, m), 4.04 (2H, t, J = 6.6 Hz), 4.99 (1H, t, J = 5.7 Hz), 5.02-5.10 (1H, m), |
| | 7.13-7.18 (1H, m), 7.21-7.27 (3H, m), 7.28-7.35 (3H, m), 7.37-7.42 (2H, m), 7.50- |
| | 7.57 (2H, m), 7.65 (1H, dt, J = 6.9, 2.1 Hz), 7.81 (1H, td, J = 1.9, 0.6 Hz), 8.60 (1H, d, J = 8.3 |
| | Hz) |
| 1009 | 1.29-1.43 (2H, m), 1.43-1.52 (4H, m), 1.74-1.98 (4H, m), 2.21-2.39 (4H, m), 3.17 (2H, |
| | t, J = 6.6 Hz), 3.41 (2H, s), 3.51 (2H, t, J = 6.8 Hz), 3.87 (2H, t, J = 6.6 Hz), 4.06 (2H, t, J = 6.6 |
| | Hz), 7.12-7.19 (1H, m), 7.21-7.29 (2H, m), 7.33 (1H, t, J = 7.7 Hz), 7.43-7.55 (2H, m), |
| | 7.55-7.65 (1H, m), 7.70-7.81 (1H, m) |
| 1010 | 1.12 (1H, s), 1.24-1.41 (6H, m), 1.48 (4H, t, J = 5.6 Hz), 1.60 (1H, d, J = 12.9 Hz), 1.75 (4H, |
| | dd, J = 22.9, 12.2 Hz), 2.32 (4H, d, J = 6.1 Hz), 3.19 (2H, t, J = 6.7 Hz), 3.40 (2H, s), 3.72- |
| | 3.82 (1H, m), 4.05 (2H, t, J = 6.6 Hz), 7.17 (1H, dt, J = 7.5, 1.4 Hz), 7.21-7.27 (2H, m), 7.33 |
| | (1H, t, J = 7.7 Hz), 7.47-7.55 (2H, m), 7.62 (1H, dt, J = 6.9, 2.1 Hz), 7.78 (1H, dt, J = 2.4, 1.2 |
| | Hz), 8.13 (1H, d, J = 8.4 Hz) |
| 1011 | 1.11 (3H, t, J = 7.1 Hz), 1.32-1.43 (2H, m), 1.41-1.54 (4H, m), 2.25-2.39 (4H, m), 3.20 |
| | (4H, t, J = 6.6 Hz), 4.06 (2H, t, J = 6.6 Hz), 7.17 (1H, s), 7.21-7.28 (2H, m), 7.33 (1H, t, J = |
| | 7.7 Hz), 7.47-7.55 (2H, m), 7.62 (1H, dt, J = 6.7, 2.2 Hz), 7.76-7.81 (1H, m), 8.45 (1H, t, |
| | J = 5.9 Hz) 2H overlapping with water peak |
| 1012 | 0.23 (2H, dd, J = 4.7, 1.7 Hz), 0.39-0.44 (2H, m), 1.00-1.09 (1H, m), 2.32 (3H, s), 2.57 |
| | (2H, t, J = 5.9 Hz), 2.79 (2H, t, J = 5.8 Hz), 3.11 (2H, t, J = 6.5 Hz), 3.18 (2H, d, J = 6.6 Hz), |
| | 3.45 (2H, s), 4.01 (2H, t, J = 6.6 Hz), 6.91 (1H, s), 7.03 (1H, d, J = 1.9 Hz), 7.10 (2H, d, J = 1.9 |
| | Hz), 7.46-7.55 (2H, m), 7.62 (1H, dt, J = 6.8, 2.1 Hz), 7.77 (1H, t, J = 1.7 Hz), 8.42 (1H, s), |
| | 8.49 (1H, t, J = 6.0 Hz) |
| 1013 | (CDCl3) 1.44 (3H, t, J = 7.1 Hz), 2.45 (3H, s), 2.69 (2H, s), 2.90 (2H, d, J = 6.1 Hz), 3.31 (2H, |
| | t, J = 6.7 Hz), 3.58 (2H, s), 4.09 (2H, t, J = 6.7 Hz), 4.47 (2H, q, J = 7.1 Hz), 6.98 (1H, d, J = |
| | 2.2 Hz), 7.07 (1H, dd, J = 8.1, 2.2 Hz), 7.13 (1H, d, J = 8.2 Hz), 7.30-7.39 (2H, m), 7.48 (1H, |
| | ddd, J = 7.6, 2.1, 1.4 Hz), 7.62 (1H, t, J = 2.0 Hz) |
| 1014 | 1.12 (3H, t, J = 7.1 Hz), 2.33 (3H, s), 2.59 (2H, t, J = 5.9 Hz), 2.81 (2H, t, J = 5.9 Hz), 3.20 |
| | (2H, t, J = 6.6 Hz), 3.25-3.29 (2H, m), 3.46 (2H, s), 4.03 (2H, t, J = 6.6 Hz), 7.04 (1H, d, J = |
| | 1.9 Hz), 7.12 (2H, t, J = 1.4 Hz), 7.48-7.56 (2H, m), 7.62 (1H, dt, J = 6.7, 2.1 Hz), 7.78 (1H, |
| | dt, J = 2.4, 1.2 Hz), 8.45 (1H, t, J = 5.9 Hz) |
| 1015 | 2.33 (3H, s), 2.59 (2H, t, J = 5.9 Hz), 2.81 (2H, t, J = 5.9 Hz), 3.20 (2H, t, J = 6.6 Hz), 3.46 |
| | (2H, s), 4.03 (2H, t, J = 6.6 Hz), 4.47 (2H, d, J = 6.2 Hz), 7.04 (1H, d, J = 1.9 Hz), 7.08-7.15 |
| | (2H, m), 7.37 (1H, ddd, J = 7.8, 4.8, 0.9 Hz), 7.47-7.57 (2H, m), 7.63 (1H, dt, J = 7.0, 2.0 |
| | Hz), 7.75 (1H, dt, J = 7.9, 1.9 Hz), 7.79 (1H, dt, J = 2.4, 1.2 Hz), 8.47 (1H, dd, J = 4.8, 1.6 |
| | Hz), 8.57 (1H, dd, J = 2.3, 0.9 Hz), 9.11 (1H, t, J = 6.3 Hz) |
| 1016 | 0.93 (1H, t, J = 7.1 Hz), 1.09-1.21 (2H, m), 1.54-1.60 (2H, m), 1.79 (2H, td, J = 11.7, 2.3 |
| | Hz), 2.11 (3H, s), 2.71 (2H, dt, J = 11.6, 3.3 Hz), 3.04 (2H, t, J = 6.7 Hz), 3.27 (2H, d, J = 6.6 |
| | Hz), 3.65 (2H, d, J = 6.7 Hz), 4.42 (2H, d, J = 6.3 Hz), 7.22 (1H, ddd, J = 8.8, 5.0, 3.8 Hz), |
| | 7.31 (3H, d, J = 4.3 Hz), 7.49-7.54 (2H, m), 7.58 (1H, dt, J = 6.3, 2.3 Hz), 7.71-7.74 (1H, |
| | m), 8.42 (3H, s), 8.96 (1H, t, J = 6.3 Hz) |
| 1017 | 1.65-1.72 (4H, m), 2.40-2.44 (4H, m), 3.21 (2H, t, J = 6.6 Hz), 3.56 (2H, s), 4.06 (2H, t, J = |
| | 6.6 Hz), 4.45 (2H, d, J = 6.3 Hz), 7.15-7.24 (1H, m), 7.20-7.32 (3H, m), 7.28-7.37 (5H, |
| | m), 7.46-7.57 (2H, m), 7.58-7.67 (1H, m), 7.77-7.82 (1H, m), 9.05 (1H, t, J = 6.4 Hz) |
| 1018 | 2.33-2.37 (4H, m), 3.21 (2H, t, J = 6.6 Hz), 3.45 (2H, s), 3.53-3.59 (4H, m), 4.07 (2H, t, J = |
| | 6.6 Hz), 4.45 (2H, d, J = 6.3 Hz), 7.19 (1H, d, J = 7.5 Hz), 7.19-7.39 (8H, m), 7.46-7.57 |
| | (2H, m), 7.60-7.67 (1H, m), 7.77-7.83 (1H, m), 9.05 (1H, t, J = 6.3 Hz) |
| 1019 | 0.06-0.21 (2H, m), 0.40-0.58 (2H, m), 0.86-0.95 (1H, m), 2.33 (2H, d, J = 1.9 Hz), 2.70 |
| | (2H, s br), 2.80 (2H, s br), 3.20 (2H, t, J = 6.6 Hz), 3.60 (2H, s br), 4.02 (2H, t, J = 6.6 Hz), |
| | 4.44 (2H, d, J = 6.3 Hz), 7.06 (1H, s), 7.11 (2H, s), 7.21-7.28 (1H, m), 7.28-7.39 (4H, m), |
| | 7.46-7.57 (2H, m), 7.63 (1H, dt, J = 6.9, 2.1 Hz), 7.76-7.83 (1H, m), 9.05 (1H, t, J = 6.4 |
| | Hz) |
| 1020 | 1.13-1.31 (2H, m), 1.39-1.60 (4H, m), 1.65-1.79 (2H, m), 2.16 (1H, quintet, J = 7.5 Hz), |
| | 2.23-2.39 (2H, m), 2.59-2.73 (2H, m), 2.78 (2H, t, J = 5.9 Hz), 3.19 (2H, t, J = 6.6 Hz), |
| | 3.51 (2H, s), 4.02 (2H, t, J = 6.6 Hz), 4.44 (2H, d, J = 6.3 Hz), 7.04 (1H, s), 7.10 (2H, d, J = 1.6 |
| | Hz), 7.20-7.28 (1H, m), 7.30-7.35 (4H, m), 7.46-7.56 (2H, m), 7.63 (1H, dt, J = 6.9, 2.0 |
| | Hz), 7.75-7.81 (1H, m), 9.05 (1H, t, J = 6.4 Hz) |
| 1021 | 1.08 (3H, t, J = 7.1 Hz), 2.58-2.65 (2H, m), 2.76-2.83 (2H, m), 3.19 (2H, t, J = 6.6 Hz), |
| | 3.51 (2H, s br), 4.02 (2H, t, J = 6.6 Hz), 4.44 (2H, d, J = 6.3 Hz), 7.06 (1H, s), 7.11 (2H, s), |
| | 7.20-7.27 (1H, m), 7.29-7.38 (4H, m), 7.44-7.58 (2H, m), 7.63 (1H, dt, J = 7.0, 2.1 Hz), |
| | 7.74-7.81 (1H, m), 9.05 (1H, t, J = 6.3 Hz) |
| 1022 | 1.04 (6H, d, J = 6.5 Hz), 2.62-2.71 (2H, m), 2.73-2.79 (2H, m), 2.84 (1H, heptet, J = 6.5 |
| | Hz), 3.20 (2H, t, J = 6.6 Hz), 3.60 (2H, s), 4.02 (2H, t, J = 6.6 Hz), 4.45 (2H, d, J = 6.3 Hz), |
| | 7.05 (1H, s), 7.09 (2H, s), 7.21-7.28 (1H, m), 7.28-7.38 (4H, m), 7.46-7.55 (2H, m), |
| | 7.63 (1H, dt, J = 6.9, 2.0 Hz), 7.76-7.83 (1H, m), 9.03 (1H, t, J = 6.3 Hz) |
| 1023 | 0.93 (2H, dd, J = 13.8, 9.2 Hz), 1.11-1.24 (3H, m), 1.51-1.63 (2H, m), 1.68 (4H, q, J = 5.9, |
| | 4.7 Hz), 2.33 (3H, s), 2.58 (2H, t, J = 5.9 Hz), 2.80 (2H, t, J = 5.8 Hz), 3.09 (2H, d, J = 6.5 Hz), |
| | 3.19 (2H, t, J = 6.6 Hz), 3.45 (2H, s), 4.02 (2H, t, J = 6.6 Hz), 6.75 (1H, s), 7.04 (1H, d, J = 1.9 |
| | Hz), 7.07-7.15 (2H, m), 7.47-7.55 (2H, m), 7.62 (1H, dt, J = 6.7, 2.1 Hz), 7.78 (1H, t, J = |
| | 2.1 Hz), 8.39 (1H, t, J = 6.2 Hz), 8.44 (1H, s) |
| 1024 | 2.32 (3H, s), 2.57 (2H, t, J = 5.9 Hz), 2.79 (2H, t, J = 5.9 Hz), 3.11-3.17 (2H, m), 3.44 (2H, |
| | s), 3.64-3.81 (2H, m), 4.00 (2H, t, J = 6.6 Hz), 5.00 (1H, t, J = 5.6 Hz), 5.06 (1H, td, J = 7.7, |
| | 5.2 Hz), 6.98-7.04 (1H, m), 7.07-7.13 (2H, m), 7.19-7.27 (1H, m), 7.29-7.35 (2H, m), |
| | 7.37-7.42 (2H, m), 7.49-7.56 (2H, m), 7.64 (1H, dt, J = 6.9, 2.0 Hz), 7.78-7.83 (1H, m), |
| | 8.61 (1H, d, J = 8.3 Hz) |
| 1025 | 0.73-0.83 (1H, m), 0.97-1.10 (3H, m), 1.50 (3H, s), 1.76 (2H, t, J = 5.9 Hz), 1.98 (2H, t, J = |
| | 5.9 Hz), 2.37 (2H, t, J = 6.6 Hz), 2.47 (2H, d, J = 6.2 Hz), 2.63 (2H, s), 2.77-2.84 (1H, m), |
| | 2.95 (1H, ddd, J = 8.2, 7.0, 5.9 Hz), 3.18 (3H, dt, J = 12.6, 6.5 Hz), 6.21 (1H, d, J = 1.9 Hz), |
| | 6.29 (2H, t, J = 1.5 Hz), 6.66-6.73 (2H, m), 6.80 (1H, dt, J = 6.8, 2.1 Hz), 6.96 (1H, dt, J = |
| | 2.4, 1.3 Hz), 7.51 (1H, t, J = 6.0 Hz) |
| 1026 | 1.61 (1H, ddd, J = 11.4, 6.2, 1.4 Hz), 1.86-1.97 (1H, m), 2.33 (3H, s), 2.58 (2H, t, J = 5.9 |
| | Hz), 2.80 (2H, t, J = 5.9 Hz), 3.19 (2H, t, J = 6.6 Hz), 3.24 (2H, t, J = 6.8 Hz), 3.45 (2H, d, J = |
| | 2.1 Hz), 3.47-3.50 (1H, m), 3.56-3.77 (4H, m), 4.02 (2H, t, J = 6.6 Hz), 7.03 (1H, d, J = 1.9 |
| | Hz), 7.08-7.14 (2H, m), 7.48-7.55 (2H, m), 7.62 (1H, dt, J = 6.9, 2.1 Hz), 7.77 (1H, t, J = |
| | 2.0 Hz), 8.61 (1H, t, J = 6.1 Hz) |
| 1027 | 1.15-1.24 (2H, m), 1.53-1.61 (2H, m), 1.75-1.88 (1H, m), 2.33 (3H, s), 2.59 (2H, t, J = |
| | 5.9 Hz), 2.81 (2H, t, J = 5.8 Hz), 3.13-3.27 (6H, m), 3.46 (2H, s), 3.84 (2H, ddd, J = 11.3, |
| | 4.3, 1.9 Hz), 4.02 (2H, t, J = 6.6 Hz), 7.04 (1H, d, J = 1.9 Hz), 7.09-7.15 (2H, m), 7.49-7.55 |
| | (2H, m), 7.62 (1H, dt, J = 6.9, 2.1 Hz), 7.78 (1H, dt, J = 2.3, 1.2 Hz), 8.49 (1H, t, J = 6.2 Hz) |
| 1028 | 2.48 (3H, s), 3.21 (2H, t, J = 6.6 Hz), 3.81 (4H, s), 4.03 (2H, t, J = 6.6 Hz), 4.45 (2H, d, J = 6.3 |
| | Hz), 7.13-7.20 (1H, m), 7.20-7.28 (3H, m), 7.31-7.34 (4H, m), 7.46-7.56 (2H, m), 7.58- |
| | 7.68 (1H, m), 7.79 (1H, s), 9.03 (1H, t, J = 6.3 Hz) |
| 1029 | 0.34-0.42 (2H, m), 0.48 (2H, dt, J = 6.1, 2.9 Hz), 1.78 (1H, tt, J = 6.7, 3.5 Hz), 2.76 (2H, t, |
| | J = 5.7 Hz), 2.83 (2H, t, J = 5.9 Hz), 3.20 (2H, t, J = 6.6 Hz), 3.68 (2H, s), 4.02 (2H, t, |
| | J = 6.6 Hz), 4.45 (2H, d, J = 6.3 Hz), 7.06 (1H, s), 7.10 (2H, d, J = 1.3 Hz), 7.21-7.28 (1H, m), |
| | 7.30-7.36 (4H, m), 7.46-7.55 (2H, m), 7.63 (1H, dt, J = 6.8, 2.1 Hz), 7.76-7.80 (1H, m), 9.03 |
| | (1H, t, J = 6.3 Hz) |
| 1030 | 1.60-1.67 (2H, m), 2.17 (3H, s), 2.78-2.85 (2H, m), 2.88 (2H, t, J = 5.2 Hz), 3.20 (2H, t, J = |
| | 6.6 Hz), 3.68 (2H, s), 4.03 (2H, t, J = 6.6 Hz), 4.45 (2H, d, J = 6.3 Hz), 7.07-7.18 (3H, m), |
| | 7.20-7.27 (1H, m), 7.29-7.36 (4H, m), 7.46-7.57 (2H, m), 7.63 (1H, dt, J = 6.9, 2.0 Hz), |
| | 7.75-7.81 (1H, m), 9.03 (1H, t, J = 6.3 Hz) |
| 1031 | 0.21-0.26 (2H, m), 0.38-0.46 (2H, m), 1.00-1.06 (1H, m), 1.08 (3H, t, J = 7.1 Hz), 2.61- |
| | 2.66 (2H, m), 2.79 (2H, t, J = 5.9 Hz), 3.12 (2H, t, J = 6.4 Hz), 3.19 (2H, t, J = 6.6 Hz), 3.51 |
| | (2H, s), 4.02 (2H, t, J = 6.6 Hz), 7.05 (1H, s), 7.11 (2H, s), 7.47-7.57 (2H, m), 7.62 (1H, dt, |
| | J = 6.9, 2.1 Hz), 7.76-7.79 (1H, m), 8.51 (1H, t, J = 6.0 Hz) |
| 1032 | 0.21-0.29 (2H, m), 0.38-0.46 (2H, m), 1.04 (1H, dddd, J = 12.6, 8.1, 5.1, 2.1 Hz), 2.52- |
| | 2.55 (2H, m), 2.81 (2H, t, J = 5.9 Hz), 3.12 (2H, t, J = 6.4 Hz), 3.19 (2H, t, J = 6.6 Hz), 3.42 |
| | (2H, s), 3.58 (1H, quintet, J = 6.3 Hz), 4.02 (2H, t, J = 6.6 Hz), 4.51 (2H, t, J = 6.1 Hz), 4.61 |
| | (2H, t, J = 6.5 Hz), 7.05 (1H, s), 7.13 (2H, s), 7.47-7.57 (2H, m), 7.62 (1H, dt, J = 6.9, 2.0 |
| | Hz), 7.75-7.80 (1H, m), 8.52 (1H, t, J = 6.0 Hz) |
| 1033 | 0.20-0.27 (2H, m), 0.38-0.45 (2H, m), 1.04 (1H, dddd, J = 11.6, 8.1, 5.1, 2.1 Hz), 2.72 |
| | (2H, t, J = 5.9 Hz), 2.84 (2H, t, J = 5.8 Hz), 3.04 (2H, s), 3.12 (2H, t, J = 6.5 Hz), 3.19 (2H, t, |
| | J = 6.7 Hz), 3.62 (2H, s), 4.02 (2H, t, J = 6.6 Hz), 7.05 (1H, s), 7.13 (2H, s), 7.15 (1H, s br), |
| | 7.24 (1H, s br), 7.48-7.55 (2H, m), 7.61-7.64 (1H, m), 7.76-7.80 (1H, m), 8.52 (1H, t, J = |
| | 6.0 Hz) |
| 1034 | 0.21-0.27 (2H, m), 0.38-0.46 (2H, m), 1.04 (1H, dddd, J = 12.7, 8.1, 5.1, 2.1 Hz), 2.37 |
| | (3H, s), 2.61 (2H, t, J = 5.8 Hz), 2.80 (2H, t, J = 5.8 Hz), 3.12 (2H, t, J = 6.5 Hz), 3.16 (2H, t, |
| | J = 6.9 Hz), 3.50 (2H, s), 4.27 (2H, t, J = 6.5 Hz), 7.46 (1H, d, J = 8.3 Hz), 7.49-7.57 (3H, m), |
| | 7.65 (1H, dt, J = 6.8, 2.1 Hz), 7.81-7.84 (1H, m), 8.52 (1H, t, J = 6.0 Hz) |
| 1035 | 0.19-0.28 (2H, m), 0.35-0.46 (2H, m), 0.97-1.12 (7H, m), 2.66-2.71 (2H, m), 2.76 (2H, |
| | t, J = 5.6 Hz), 2.79-2.90 (1H, m), 3.12 (2H, t, J = 6.4 Hz), 3.19 (2H, t, J = 6.6 Hz), 3.60 (2H, |
| | s), 4.02 (2H, t, J = 6.6 Hz), 7.02-7.14 (3H, m), 7.46-7.57 (2H, m), 7.57-7.66 (1H, m), |
| | 7.75-7.80 (1H, m), 8.52 (1H, t, J = 6.0 Hz) |
| 1036 | 0.05-0.13 (2H, m), 0.16-0.24 (2H, m), 0.33-0.42 (2H, m), 0.42-0.50 (2H, m), 0.85- |
| | 0.88 (1H, m), 0.95-1.06 (1H, m), 2.27-2.33 (2H, m), 2.59-2.70 (2H, m), 2.76 (2H, t, J = |
| | 5.8 Hz), 3.08 (2H, t, J = 6.4 Hz), 3.15 (2H, t, J = 6.6 Hz), 3.56 (2H, s), 3.98 (2H, t, J = 6.6 Hz), |
| | 7.00-7.11 (3H, m), 7.42-7.53 (2H, m), 7.53-7.62 (1H, m), 7.74 (1H, t, J = 2.0 Hz), 8.47 |
| | (1H, t, J = 6.0 Hz) |
| 1037 | 0.19-0.27 (2H, m), 0.35-0.46 (2H, m), 1.04 (1H, s), 2.60-2.73 (4H, m), 2.78 (2H, t, J = |
| | 5.8 Hz), 3.08-3.23 (4H, m), 3.25 (3H, s), 3.51 (2H, t, J = 5.7 Hz), 3.57 (2H, s), 4.02 (2H, t, |
| | J = 6.6 Hz), 7.03 (1H, s), 7.10 (2H, s), 7.46-7.57 (2H, m), 7.57-7.66 (1H, m), 7.77 (1H, d, J = |
| | 2.1 Hz), 8.52 (1H, t, J = 6.0 Hz) |
| 1038 | 0.93-0.99 (3H, m), 1.19-1.28 (1H, m), 1.89-2.01 (2H, m), 2.09-2.22 (1H, m), 2.36- |
| | 2.46 (1H, m), 2.50-2.60 (1H, m), 2.65-2.73 (1H, m), 3.21 (2H, t, J = 6.6 Hz), 3.56 (1H, d, |
| | J = 13.2 Hz), 4.06 (2H, t, J = 6.6 Hz), 4.45 (2H, d, J = 6.3 Hz), 7.16-7.29 (5H, m), 7.30-7.37 |
| | (5H, m), 7.46-7.57 (2H, m), 7.58-7.67 (1H, m), 7.79 (1H, t, J = 1.9 Hz), 9.03 (1H, t, J = 6.4 |
| | Hz) |
| 1039 | 1.05-1.11 (3H, m), 1.21-1.39 (1H, m), 1.53-1.65 (2H, m), 1.83-1.96 (1H, m), 2.02 (1H, |
| | q, J = 8.8 Hz), 2.33-2.45 (1H, m), 2.73-2.83 (1H, m), 3.09 (1H, d, J = 13.2 Hz), 3.21 (2H, t, |
| | J = 6.6 Hz), 3.96 (1H, d, J = 13.3 Hz), 4.06 (2H, t, J = 6.7 Hz), 4.45 (2H, d, J = 6.3 Hz), 7.18 |
| | (1H, d, J = 7.6 Hz), 7.18-7.28 (3H, m), 7.28-7.37 (5H, m), 7.46-7.57 (2H, m), 7.58-7.67 |
| | (1H, m), 7.77-7.83 (1H, m), 9.05 (1H, t, J = 6.3 Hz) |
| 1040 | 0.20-0.28 (2H, m), 0.36-0.46 (2H, m), 0.97-1.12 (1H, m), 2.36 (3H, s), 2.69 (2H, t, J = |
| | 6.0 Hz), 2.88 (2H, t, J = 6.0 Hz), 3.12 (2H, t, J = 6.4 Hz), 3.22 (2H, t, J = 6.6 Hz), 3.50 (2H, s), |
| | 4.07 (2H, t, J = 6.6 Hz), 7.47-7.58 (3H, m), 7.58-7.67 (1H, m), 7.79 (1H, t, J = 1.7 Hz), 8.37 |
| | (1H, d, J = 2.5 Hz), 8.53 (1H, t, J = 6.1 Hz) |
| 1041 | 2.32 (3H, s), 2.58 (2H, d, J = 5.9 Hz), 2.79 (2H, t, J = 5.9 Hz), 3.16 (2H, t, J = 6.6 Hz), 3.45 |
| | (2H, d, J = 2.2 Hz), 4.01 (2H, t, J = 6.6 Hz), 4.61-4.74 (4H, m), 4.99-5.07 (1H, m), 7.01- |
| | 7.04 (1H, m), 7.07-7.14 (2H, m), 7.49-7.56 (2H, m), 7.64 (1H, dt, J = 7.0, 2.0 Hz), 7.79 |
| | (1H, td, J = 1.9, 0.7 Hz), 9.20 (1H, d, J = 6.8 Hz) |
| 1042 | 1.92-2.02 (1H, m), 2.09-2.21 (1H, m), 2.33 (3H, s), 2.59 (2H, t, J = 5.9 Hz), 2.81 (2H, t, J = |
| | 5.9 Hz), 3.19 (2H, t, J = 6.6 Hz), 3.46 (2H, s), 3.59 (1H, dd, J = 8.8, 4.9 Hz), 3.66-3.76 (1H, |
| | m), 3.81-3.89 (2H, m), 4.03 (2H, t, J = 6.6 Hz), 4.42-4.53 (1H, m), 7.04 (1H, d, J = 1.9 Hz), |
| | 7.12 (2H, d, J = 2.3 Hz), 7.48-7.55 (2H, m), 7.63 (1H, dt, J = 7.0, 2.0 Hz), 7.79 (1H, t, J = 2.0 |
| | Hz), 8.52 (1H, d, J = 7.0 Hz) |
| 1043 | 1.66 (4H, dq, J = 11.3, 4.6 Hz), 2.31 (3H, s), 2.57 (2H, t, J = 5.8 Hz), 2.79 (2H, t, J = 5.9 Hz), |
| | 3.17 (2H, t, J = 6.6 Hz), 3.37 (2H, dd, J = 11.3, 3.4 Hz), 3.44 (2H, s), 3.86 (2H, dt, J = 11.7, |
| | 3.5 Hz), 4.01 (3H, t, J = 6.5 Hz), 7.00-7.04 (1H, m), 7.07-7.14 (2H, m), 7.46-7.55 (2H, |
| | m), 7.61 (1H, dt, J = 7.0, 2.0 Hz), 7.76 (1H, dt, J = 2.4, 1.2 Hz), 8.33 (1H, d, J = 8.3 Hz) |
| 1044 | 1.50-1.61 (4H, m), 1.64-1.73 (2H, m), 1.82-1.93 (2H, m), 2.33 (3H, s), 2.58 (2H, t, J = |
| | 5.9 Hz), 2.80 (2H, t, J = 5.9 Hz), 3.18 (2H, t, J = 6.6 Hz), 3.45 (2H, s), 4.02 (2H, t, J = 6.6 Hz), |
| | 4.18-4.28 (1H, m, 7.02-7.06 (1H, m), 7.11 (2H, t, J = 1.5 Hz), 7.47-7.55 (2H, m), 7.62 |
| | (1H, dt, J = 6.9, 2.1 Hz), 7.78 (1H, dt, J = 2.4, 1.2 Hz), 8.24 (1H, d, J = 7.8 Hz) |
| 1045 | 2.32 (3H, s), 2.38-2.46 (1H, m), 2.58 (3H, d, J = 5.7 Hz), 2.79 (2H, t, J = 5.9 Hz), 3.18 (2H, t, |
| | J = 6.6 Hz), 3.44 (2H, s), 3.51 (2H, d, J = 19.6 Hz), 4.02 (2H, t, J = 6.6 Hz), 4.37-4.54 (2H, |
| | m), 4.81 (1H, dq, J = 7.4, 5.7 Hz), 7.01-7.05 (1H, m), 7.11 (2H, t, J = 1.5 Hz), 7.46-7.54 |
| | (2H, m), 7.62 (1H, dt, J = 6.8, 2.1 Hz), 7.77 (1H, td, J = 1.9, 0.6 Hz), 8.47 (1H, t, J = 6.1 Hz) |
| 1046 | 1.56 (2H, dd, J = 7.1, 2.6 Hz), 1.72-1.83 (4H, m), 2.31 (3H, s), 2.58 (2H, d, J = 5.8 Hz), 2.79 |
| | (2H, t, J = 5.9 Hz), 3.15-3.20 (4H, m), 3.28 (2H, d, J = 6.3 Hz), 3.44 (2H, s), 3.99 (4H, dt, J = |
| | 21.5, 6.7 Hz), 4.52 (1H, t, J = 5.5 Hz), 7.01-7.03 (1H, m), 7.07-7.14 (2H, m), 7.47-7.54 |
| | (2H, m), 7.61 (1H, dt, J = 6.6, 2.1 Hz), 7.77 (1H, td, J = 1.9, 0.7 Hz), 8.11 (1H, t, J = 6.3 Hz) |
| 1047 | 2.34 (3H, s), 2.60 (2H, d, J = 5.8 Hz), 2.80 (2H, t, J = 5.9 Hz), 3.19 (3H, t, J = 6.7 Hz), 3.45- |
| | 3.56 (4H, m), 4.02 (2H, t, J = 6.6 Hz), 4.35 (2H, t, J = 6.0 Hz), 4.62 (2H, dd, J = 7.8, 6.0 Hz), |
| | 7.04 (1H, d, J = 1.9 Hz), 7.08-7.15 (2H, m), 7.47-7.56 (2H, m), 7.61 (1H, dt, J = 7.1, 1.9 |
| | Hz), 7.77 (1H, t, J = 1.7 Hz), 8.68 (1H, t, J = 6.0 Hz) |
| 1048 | 0.19-0.28 (2H, m), 0.35-0.46 (2H, m), 0.97-1.12 (1H, m), 2.16 (3H, s), 2.32 (3H, s), 2.59-2.68 |
| | (4H, m), 3.11 (2H, t, J = 6.5 Hz), 3.18 (2H, t, J = 6.7 Hz), 3.43 (2H, s), 4.00 (2H, t, J = |
| | 6.6 Hz), 6.84-6.89 (1H, m), 6.96-7.01 (1H, m), 7.45-7.56 (2H, m), 7.56-7.65 (1H, m), |
| | 7.74-7.79 (1H, m), 8.52 (1H, t, J = 6.0 Hz) |
| 1049 | 1.67-1.83 (6H, m), 2.32 (3H, s), 2.57 (2H, t, J = 5.9 Hz), 2.79 (2H, t, J = 5.9 Hz), 3.18 (2H, t, |
| | J = 6.6 Hz), 3.38 (4H, dd, J = 6.0, 3.5 Hz), 3.44 (2H, s), 4.02 (2H, t, J = 6.6 Hz), 4.74 (1H, t, |
| | J = 5.7 Hz), 7.01-7.04 (1H, m), 7.06-7.15 (2H, m), 7.47-7.54 (2H, m), 7.60 (1H, dt, J = 6.9, |
| | 2.1 Hz), 7.76 (1H, td, J = 1.9, 0.6 Hz), 8.37 (1H, t, J = 6.2 Hz) |
| 1050 | 1.35 (2H, dd, J = 4.5, 1.8 Hz), 1.60 (2H, dd, J = 4.5, 1.8 Hz), 2.31 (3H, s), 2.57 (2H, t, J = 5.9 |
| | Hz), 2.79 (2H, t, J = 5.9 Hz), 3.17 (2H, t, J = 6.6 Hz), 3.44 (2H, s), 3.49-3.59 (6H, m), 4.01 |
| | (2H, t, J = 6.6 Hz), 4.64 (1H, t, J = 5.9 Hz), 7.02 (1H, d, J = 1.9 Hz), 7.06-7.17 (2H, m), 7.46- |
| | 7.55 (2H, m), 7.61 (1H, dt, J = 6.8, 2.1 Hz), 7.77 (1H, dt, J = 2.3, 1.2 Hz), 8.61 (1H, t, J = 6.3 |
| | Hz) |
| 1051 | 1.07 (3H, t, J = 7.1 Hz), 2.45 (2H, d, J = 7.1 Hz), 2.62 (2H, t, J = 5.9 Hz), 2.78 (2H, t, J = 5.8 |
| | Hz), 3.13-3.17 (2H, m), 3.50 (2H, s), 3.65-3.78 (2H, m), 4.00 (2H, t, J = 6.6 Hz), 5.05 (2H, |
| | td, J = 7.8, 5.2 Hz), 6.86 (1H, s), 7.04 (1H, d, J = 1.8 Hz), 7.09 (2H, d, J = 1.9 Hz), 7.21-7.25 |
| | (1H, m), 7.32 (2H, t, J = 7.5 Hz), 7.37-7.41 (2H, m), 7.48-7.57 (2H, m), 7.64 (1H, dt, J = |
| | 6.9, 2.0 Hz), 7.79 (1H, t, J = 2.0 Hz), 8.43 (1H, s), 8.61 (1H, d, J = 8.3 Hz) |
| 1052 | 0.19-0.28 (2H, m), 0.37-0.46 (2H, m), 0.88 (3H, t, J = 7.4 Hz), 1.00-1.10 (1H, m), 1.45- |
| | 1.59 (2H, m), 2.35-2.42 (2H, m), 2.63 (2H, t, J = 5.9 Hz), 2.78 (2H, t, J = 5.8 Hz), 3.12 (2H, |
| | t, J = 6.5 Hz), 3.19 (2H, t, J = 6.6 Hz), 3.28-3.32 (1H, m), 3.50 (2H, s), 4.02 (2H, t, J = 6.6 |
| | Hz), 7.05 (1H, s), 7.10 (2H, d, J = 1.5 Hz), 7.46-7.57 (2H, m), 7.57-7.66 (1H, m), 7.75- |
| | 7.80 (1H, m), 8.52 (1H, t, J = 6.0 Hz) |
| 1053 | 0.19-0.28 (2H, m), 0.35-0.46 (2H, m), 0.97-1.12 (1H, m), 1.68-1.79 (2H, m), 2.43- |
| | 2.50 (2H, m), 2.63 (2H, t, J = 5.8 Hz), 2.78 (2H, t, J = 5.8 Hz), 3.12 (2H, t, J = 6.5 Hz), 3.15- |
| | 3.24 (5H, m), 3.37 (2H, t, J = 6.4 Hz), 3.51 (2H, s), 4.02 (2H, t, J = 6.6 Hz), 7.04 (1H, d, J = |
| | 1.7 Hz), 7.10 (2H, d, J = 1.4 Hz), 7.46-7.57 (2H, m), 7.62 (1H, dt, J = 6.9, 2.1 Hz), 7.77 (1H, |
| | t, J = 1.7 Hz), 8.51 (1H, t, J = 6.0 Hz) |
| 1054 | 0.20-0.28 (2H, m), 0.36-0.46 (2H, m), 0.99-1.14 (4H, m), 2.10 (3H, s), 2.47 (2H, t, J = |
| | 7.1 Hz), 2.55-2.68 (2H, m), 2.76 (2H, t, J = 5.9 Hz), 3.12 (2H, t, J = 6.5 Hz), 3.13-3.32 (2H, |
| | m), 3.42-3.55 (2H, m), 3.68-3.78 (1H, m), 3.97-4.08 (1H, m), 6.99 (2H, d, J = 7.4 Hz), |
| | 7.46-7.56 (2H, m), 7.57-7.67 (1H, m), 7.74-7.80 (1H, m), 8.52 (1H, t, J = 6.0 Hz) |
| 1055 | 0.21-0.28 (2H, m), 0.38-0.45 (2H, m), 1.00-1.10 (1H, m), 2.55 (2H, t, J = 6.2 Hz), 2.69 |
| | (2H, t, J = 5.8 Hz), 2.78 (2H, t, J = 5.7 Hz), 3.12 (2H, t, J = 6.5 Hz), 3.19 (2H, t, J = 6.6 Hz), |
| | 3.54-3.61 (4H, m), 4.02 (2H, t, J = 6.6 Hz), 4.47 (1H, t, J = 5.4 Hz), 7.04 (1H, s), 7.11 (2H, |
| | s), 7.48-7.55 (2H, m), 7.62 (1H, dt, J = 6.8, 2.1 Hz), 7.76-7.80 (1H, m), 8.51 (1H, t, J = 6.0 |
| | Hz) |
| 1056 | 0.21-0.27 (2H, m), 0.38-0.45 (2H, m), 1.00-1.06 (1H, m), 1.08 (3H, t, J = 7.1 Hz), 2.54 |
| | (2H, q, J = 7.3 Hz), 2.70 (2H, t, J = 5.7 Hz), 2.80 (2H, t, J = 5.8 Hz), 3.11 (2H, t, J = 6.5 Hz), |
| | 3.18 (2H, t, J = 6.4 Hz), 3.53 (2H, s), 4.19 (2H, t, J 6.5 Hz), 7.49-7.58 (2H, m), 7.62 (1H, dt, |
| | J = 7.3, 1.8 Hz), 7.79 (1H, t, J = 2.0 Hz), 8.52 (1H, t, J = 6.1 Hz), 8.54 (1H, s) |
| 1057 | 1.68-1.83 (1H, m), 1.90 (1H, dq, J = 10.6, 3.8 Hz), 2.38 (2H, d, J = 8.0 Hz), 2.85 (3H, s), |
| | 3.14-3.25 (5H, m), 4.03 (2H, t, J = 6.6 Hz), 4.16-4.31 (3H, m), 7.19 (1H, d, J = 5.5 Hz), |
| | 7.27 (2H, s), 7.41-7.57 (4H, m), 7.62 (1H, dt, J = 7.1, 2.0 Hz), 7.78 (1H, t, J = 2.0 Hz), 8.47 |
| | (1H, d, J = 8.0 Hz), 9.22 (2H, s) |
| 1058 | 0.13-0.30 (2H, m), 0.30-0.43 (2H, m), 0.83 (1H, ddd, J = 12.8, 7.9, 3.1 Hz), 2.33 (3H, s), |
| | 2.58 (2H, t, J = 5.9 Hz), 2.80 (2H, t, J = 5.9 Hz), 3.16-3.31 (4H, m), 3.41-3.52 (3H, m), 4.02 |
| | (2H, t, J = 6.6 Hz), 4.83 (1H, s), 7.02-7.05 (1H, m), 7.11 (2H, t, J = 1.4 Hz), 7.48-7.55 (2H, |
| | m), 7.62 (1H, dt, J = 6.7, 2.1 Hz), 7.77 (1H, td, J = 1.9, 0.7 Hz), 8.13 (1H, t, J = 5.9 Hz) |
| 1059 | 0.21-0.26 (2H, m), 0.39-0.45 (2H, m), 0.87 (3H, t, J = 7.4 Hz), 0.98 (3H, d, J = 6.5 Hz), |
| | 1.01-1.11 (1H, m), 1.34 (1H, dt, J = 14.0, 7.2 Hz), 1.56 (1H, dt, J = 13.9, 6.9 Hz), 2.61 (2H, |
| | q, J = 6.8 Hz), 2.71-2.78 (3H, m), 3.12 (2H, t, J = 6.4 Hz), 3.19 (2H, t, J = 6.6 Hz), 3.56 (1H, |
| | d, J = 15.1 Hz), 3.65 (1H, d, J = 15.1 Hz), 4.02 (2H, t, J = 6.6 Hz), 7.04 (1H, s), 7.09 (2H, d, J = |
| | 1.9 Hz), 7.48-7.57 (2H, m), 7.62 (1H, dt, J = 6.8, 2.1 Hz), 7.76-7.79 (1H, m), 8.51 (1H, t, |
| | J = 6.0 Hz) |
| 1060 | 0.21-0.27 (2H, m), 0.39-0.45 (2H, m), 1.02 (3H, d, J = 6.7 Hz), 1.04-1.10 (1H, m), 2.72- |
| | 2.79 (4H, m), 2.92 (1H, q, J = 6.3 Hz), 3.12 (2H, t, J = 6.5 Hz), 3.19 (2H, t, J = 6.7 Hz), 3.25 |
| | (3H, s), 3.30 (1H, dd, J = 9.7, 6.1 Hz), 3.48 (1H, dd, J = 9.7, 5.8 Hz), 3.69 (2H, s), 4.01 (2H, t, |
| | J = 6.6 Hz), 7.03 (1H, s), 7.09 (2H, s), 7.48-7.54 (2H, m), 7.60-7.64 (1H, m), 7.76-7.80 |
| | (1H, m), 8.51 (1H, t, J = 6.0 Hz) |
| 1061 | 0.21-0.26 (2H, m), 0.39-0.45 (2H, m), 1.00-1.07 (1H, m), 1.10 (3H, t, J = 7.0 Hz), 2.63 |
| | (2H, t, J = 5.9 Hz), 2.70 (2H, t, J = 5.8 Hz), 2.77 (2H, t, J = 5.8 Hz), 3.12 (2H, t, J = 6.4 Hz), |
| | 3.19 (2H, t, J = 6.6 Hz), 3.43 (2H, q, J = 7.0 Hz), 3.54 (2H, t, J = 5.9 Hz), 3.58 (2H, s), 4.02 |
| | (2H, t, J = 6.6 Hz), 7.03 (1H, s), 7.10 (2H, d, J = 1.3 Hz), 7.48-7.56 (2H, m), 7.62 (1H, dt, J = |
| | 6.9, 2.0 Hz), 7.77-7.78 (1H, m), 8.51 (1H, t, J = 6.0 Hz) |
| 1063 | 2.31 (3H, s), 2.56 (2H, t, J = 5.9 Hz), 2.78 (2H, t, J = 5.9 Hz), 3.11-3.18 (2H, m), 3.40-3.45 |
| | (2H, m), 3.64-3.78 (2H, m), 3.99 (2H, t, J = 6.6 Hz), 4.97-5.09 (2H, m), 7.00-7.04 (1H, |
| | m), 7.06-7.13 (2H, m), 7.21-7.26 (1H, m), 7.31 (2H, ddd, J = 7.7, 6.8, 1.2 Hz), 7.36-7.41 |
| | (2H, m), 7.50-7.56 (2H, m), 7.63 (1H, dt, J = 6.9, 2.1 Hz), 7.79 (1H, td, J = 2.0, 0.6 Hz), 8.61 |
| | (1H, d, J = 8.3 Hz) |
| 1064 | 1.42 (3H, s), 1.59 (2H, ddd, J = 13.6, 9.3, 4.3 Hz), 2.22 (2H, dd, J = 10.3, 6.8 Hz), 2.32 (3H, |
| | s), 2.57 (2H, t, J = 5.9 Hz), 2.79 (2H, t, J = 5.9 Hz), 3.17 (2H, t, J = 6.6 Hz), 3.44 (2H, s), |
| | 3.50-3.66 (4H, m), 4.01 (2H, t, J = 6.6 Hz), 7.02 (1H, d, J = 2.0 Hz), 7.07-7.13 (2H, m), 7.49- |
| | 7.54 (3H, m), 7.62 (1H, dt, J = 6.8, 2.1 Hz), 7.78 (1H, dt, J = 2.4, 1.3 Hz) |
| 1065 | 2.15-2.20 (3H, m), 2.33 (3H, s), 2.58 (2H, t, J = 5.9 Hz), 2.80 (2H, t, J = 5.9 Hz), 3.20 (2H, t, |
| | J = 6.6 Hz), 3.46 (2H, s), 4.03 (2H, t, J = 6.6 Hz), 4.36 (2H, s), 5.83-5.97 (1H, m), 7.04 (1H, |
| | dd, J = 1.8, 0.9 Hz), 7.12 (2H, d, J = 1.8 Hz), 7.48-7.55 (2H, m), 7.63 (1H, dt, J = 6.7, 2.1 |
| | Hz), 7.78 (1H, td, J = 1.9, 0.7 Hz), 8.65 (1H, s), 12.21 (1H, s) |
| 1066 | 2.33 (3H, s), 2.58 (2H, t, J = 5.9 Hz), 2.80 (2H, s), 2.96 (2H, s), 3.32 (3H, s), 3.45 (2H, s), |
| | 4.01 (2H, t, J = 6.6 Hz), 4.51 (2H, s), 7.04 (1H, d, J = 1.8 Hz), 7.11 (2H, d, J = 1.8 Hz), 7.46- |
| | 7.48 (2H, m), 7.51-7.56 (1H, m), 7.66 (1H, dt, J = 2.5, 1.0 Hz) |
| 1067 | 2.32 (3H, s), 2.57 (2H, t, J = 5.9 Hz), 2.79 (2H, t, J = 5.9 Hz), 3.08 (2H, t, J = 6.6 Hz), 3.44 |
| | (2H, s), 3.64 (6H, d, J = 17.9 Hz), 3.92 (2H, s), 4.02 (2H, t, J = 6.6 Hz), 7.02 (1H, d, J = 2.0 |
| | Hz), 7.08-7.14 (2H, m), 7.48-7.53 (2H, m), 7.55-7.60 (1H, m), 7.73 (1H, dt, J = 2.5, 1.3 |
| | Hz) |
| 1068 | 2.31 (3H, s), 2.57 (2H, t, J = 5.9 Hz), 2.79 (2H, t, J = 5.9 Hz), 3.19 (2H, t, J = 6.6 Hz), 3.44 |
| | (2H, s), 4.01 (2H, t, J = 6.7 Hz), 4.27 (2H, d, J = 6.1 Hz), 6.47 (1H, dd, J = 1.8, 0.9 Hz), 7.02 |
| | (1H, d, J = 1.9 Hz), 7.10 (2H, t, J = 1.4 Hz), 7.48-7.52 (2H, m), 7.55-7.58 (2H, m), 7.61 |
| | (1H, dt, J = 6.9, 2.1 Hz), 7.74-7.82 (1H, m), 8.81 (1H, t, J = 6.1 Hz) |
| 1070 | 0.21-0.28 (2H, m), 0.39-0.45 (2H, m), 1.01-1.06 (1H, m), 1.07 (3H, d, J = 6.1 Hz), 2.30- |
| | 2.37 (1H, m), 2.39-2.46 (1H, m), 2.69-2.73 (2H, m), 2.77-2.83 (2H, m), 3.12 (2H, t, J = |
| | 6.4 Hz), 3.19 (2H, t, J = 6.6 Hz), 3.59 (2H, s br), 3.82-3.88 (1H, m), 4.02 (2H, t, J = 6.6 Hz), |
| | 4.38 (1H, s br), 7.04 (1H, s), 7.11 (2H, d, J = 1.3 Hz), 7.46-7.55 (2H, m), 7.62 (1H, dt, J = |
| | 6.8, 2.3 Hz), 7.76-7.80 (1H, m), 8.50 (1H, t, J = 6.0 Hz) |
| 1071 | 0.21-0.27 (2H, m), 0.38-0.45 (2H, m), 0.99-1.10 (1H, m), 2.81 (2H, t, J = 5.8 Hz), 2.92 |
| | (2H, t, J = 5.9 Hz), 3.12 (2H, t, J = 6.4 Hz), 3.19 (2H, t, J = 6.7 Hz), 3.37 (2H, q, J = 10.1 Hz), |
| | 3.79 (2H, s), 4.02 (2H, t, J = 6.6 Hz), 7.06 (1H, s), 7.13 (2H, d, J = 1.3 Hz), 7.47-7.56 (2H, |
| | m), 7.62 (1H, dt, J = 6.7, 2.1 Hz), 7.76-7.79 (1H, m), 8.51 (1H, t, J = 6.0 Hz) |
| 1072 | 0.20-0.28 (2H, m), 0.37-0.46 (2H, m), 1.00-1.09 (1H, m), 1.65-1.73 (4H, m), 2.38- |
| | 2.44 (4H, m), 3.12 (2H, t, J = 6.5 Hz), 3.20 (2H, t, J = 6.6 Hz), 3.56 (2H, s), 4.06 (2H, t, J = |
| | 6.6 Hz), 7.15-7.26 (2H, m), 7.26-7.37 (2H, m), 7.46-7.57 (2H, m), 7.59-7.66 (1H, m), |
| | 7.76-7.81 (1H, m), 8.51 (1H, t, J = 6.0 Hz) |
| 1073 | 0.20-0.28 (2H, m), 0.25-0.35 (1H, m), 0.37-0.46 (2H, m), 0.63-0.70 (1H, m), 0.98- |
| | 1.12 (1H, m), 1.30-1.38 (2H, m), 2.26-2.32 (2H, m), 2.83-2.89 (2H, m), 3.12 (2H, t, J = |
| | 6.5 Hz), 3.20 (2H, t, J = 6.6 Hz), 3.56 (2H, s), 4.05 (2H, t, J = 6.6 Hz), 7.10-7.17 (1H, m), |
| | 7.19-7.25 (2H, m), 7.28-7.36 (1H, m), 7.46-7.57 (2H, m), 7.58-7.67 (1H, m), 7.76- |
| | 7.82 (1H, m), 8.50 (1H, t, J = 6.0 Hz) |
| 1074 | 0.20-0.28 (2H, m), 0.37-0.46 (2H, m), 0.99-1.10 (1H, m), 2.98 (2H, t, J = 6.2 Hz), 3.12 |
| | (2H, t, J = 6.4 Hz), 3.22 (2H, t, J = 6.6 Hz), 3.33-3.42 (2H, m), 4.04 (2H, t, J = 6.6 Hz), 4.24 |
| | (2H, s), 7.22-7.31 (3H, m), 7.47-7.57 (2H, m), 7.59-7.66 (1H, m), 7.75-7.81 (1H, m), |
| | 8.53 (1H, t, J = 6.0 Hz), 9.04 (1H, s) |
| 1075 | 1.41-1.58 (4H, m), 1.76 (1H, d, J = 12.4 Hz), 1.89 (1H, dd, J = 12.5, 8.1 Hz), 2.33 (3H, s), |
| | 2.58 (2H, t, J = 5.9 Hz), 2.80 (2H, t, J = 5.9 Hz), 3.14-3.22 (2H, m), 3.45 (2H, s), 3.94-4.06 |
| | (3H, m), 4.39 (1H, d, J = 5.0 Hz), 4.56 (1H, t, J = 4.7 Hz), 7.04 (1H, d, J = 1.8 Hz), 7.11 (2H, |
| | d, J = 2.1 Hz), 7.48-7.54 (2H, m), 7.64 (1H, dt, J = 6.8, 2.1 Hz), 7.80 (1H, t, J = 2.0 Hz), 8.01 |
| | (1H, d, J = 6.8 Hz) |
| 1076 | 1.59-1.75 (4H, m), 2.54 (2H, t, J = 6.2 Hz), 2.64-2.71 (2H, m), 2.77 (2H, t, J = 5.7 Hz), 3.17 |
| | (2H, t, J = 6.6 Hz), 3.37 (1H, dd, J = 11.2, 3.3 Hz), 3.51-3.60 (4H, m), 3.86 (2H, dt, J = 11.6, |
| | 3.4 Hz), 4.00 (4H, t, J = 6.7 Hz), 4.45 (1H, t, J = 5.4 Hz), 7.02 (1H, d, J = 1.8 Hz), 7.09 (2H, d, |
| | J = 1.8 Hz), 7.46-7.55 (2H, m), 7.61 (1H, dt, J = 7.0, 2.0 Hz), 7.76 (1H, dt, J = 2.3, 1.2 Hz), |
| | 8.32 (1H, d, J = 8.3 Hz) |
| 1077 | 2.17 (3H, s), 2.54 (2H, t, J = 6.2 Hz), 2.68 (2H, dd, J = 7.6, 4.1 Hz), 2.77 (2H, d, J = 5.8 Hz), |
| | 3.19 (2H, t, J = 6.6 Hz), 3.54-3.60 (4H, m), 4.01 (2H, t, J = 6.6 Hz), 4.35 (2H, s), 4.45 (1H, t, |
| | J = 5.4 Hz), 5.90 (1H, s), 7.03 (1H, t, J = 1.3 Hz), 7.10 (2H, d, J = 1.3 Hz), 7.46-7.52 (2H, m), |
| | 7.61 (1H, dt, J = 6.6, 2.2 Hz), 7.77 (1H, td, J = 1.9, 0.7 Hz), 8.65 (1H, s), 12.20 (1H, s) |
| 1078 | 2.55 (2H, t, J = 6.2 Hz), 2.67-2.73 (2H, m), 2.79 (2H, t, J = 6.7 Hz), 3.17-3.23 (2H, m), |
| | 3.54-3.62 (4H, m), 4.02 (2H, t, J = 6.6 Hz), 4.44-4.50 (3H, m), 7.02-7.06 (1H, m), 7.11 (2H, d, |
| | J = 1.7 Hz), 7.37 (1H, ddd, J = 7.9, 4.8, 0.9 Hz), 7.47-7.56 (2H, m), 7.63 (1H, dt, J = 7.0, 2.0 |
| | Hz), 7.72-7.81 (2H, m), 8.47 (1H, dd, J = 4.8, 1.7 Hz), 8.56 (1H, dd, J = 2.3, 0.8 Hz), 9.12 |
| | (1H, t, J = 6.3Hz) |
| 1079 | 0.20-0.28 (2H, m), 0.37-0.46 (2H, m), 1.02-1.12 (4H, m), 2.61-2.66 (2H, m), 2.78- |
| | 2.83 (2H, m), 3.12 (2H, t, J = 6.5 Hz), 3.21 (2H, t, J = 6.6 Hz), 3.26-3.31 (2H, m), 3.45-3.52 |
| | (2H, m), 3.96 (2H, t, J = 6.6 Hz), 7.04 (1H, d, J = 10.9 Hz), 7.16 (1H, d, J = 7.7 Hz), 7.47- |
| | 7.57 (2H, m), 7.58-7.65 (1H, m), 7.77 (1H, d, J = 2.3 Hz), 8.52 (1H, t, J = 6.0 Hz) |
| 1080 | 0.20-0.28 (2H, m), 0.37-0.46 (2H, m), 1.02-1.07 (1H, m), 1.80-1.95 (2H, m), 2.52- |
| | 2.56 (2H, m), 2.64-2.69 (2H, m), 2.77-2.82 (2H, m), 3.12 (2H, t, J = 6.5 Hz), 3.19 (2H, t, |
| | J = 6.6 Hz), 3.51-3.56 (2H, m), 4.02 (2H, t, J = 6.6 Hz), 4.51 (2H, dt, J = 47.5, 6.0 Hz), 7.05 |
| | (1H, s), 7.09-7.14 (2H, m), 7.46-7.56 (2H, m), 7.58-7.65 (1H, m), 7.77 (1H, d, J = 2.6 |
| | Hz), 8.50 (1H, t, J = 6.0 Hz) |
| 1081 | 0.19-0.28 (2H, m), 0.37-0.46 (2H, m), 0.98-1.12 (1H, m), 1.79 (3H, s), 2.65-2.70 (2H, |
| | m), 2.77-2.82 (2H, m), 3.12 (2H, t, J = 6.4 Hz), 3.16-3.26 (4H, m), 3.28-3.31 (2H, m), |
| | 3.54-3.58 (2H, m), 4.02 (2H, t, J = 6.6 Hz), 7.06 (1H, s), 7.09-7.14 (2H, m), 7.46-7.56 |
| | (2H, m), 7.58-7.66 (1H, m), 7.75-7.82 (2H, m), 8.50 (1H, t, J = 6.0 Hz) |
| 1082 | 0.19-0.28 (2H, m), 0.37-0.46 (2H, m), 1.02-1.12 (4H, m), 1.24 (6H, s), 2.34-2.39 (2H, |
| | m), 2.42-2.49 (2H, m), 3.12 (2H, t, J = 6.4 Hz), 3.18 (2H, t, J = 6.7 Hz), 3.47-3.51 (2H, m), |
| | 4.02 (2H, t, J = 6.7 Hz), 7.01 (1H, s), 7.14 (1H, d, J = 8.3 Hz), 7.35 (1H, d, J = 8.3 Hz), 7.46- |
| | 7.56 (2H, m), 7.58-7.65 (1H, m), 7.77 (1H, d, J = 2.3 Hz), 8.50 (1H, t, J = 6.1 Hz) |
| 1083 | 0.21-0.27 (2H, m), 0.39-0.46 (2H, m), 1.00-1.11 (1H, m), 2.58 (2H, t, J = 6.6 Hz), 2.68- |
| | 2.72 (2H, m), 2.79 (2H, t, J = 5.9 Hz), 2.92 (3H, s), 3.10-3.16 (4H, m), 3.20 (2H, t, J = 6.6 |
| | Hz), 3.58 (2H, s), 4.02 (2H, t, J = 6.6 Hz), 6.93 (1H, t, J = 5.9 Hz), 7.05 (1H, s), 7.12 (2H, d, |
| | J = 1.7 Hz), 7.47-7.56 (2H, m), 7.62 (1H, dt, J = 6.8, 2.1 Hz), 7.75-7.79 (1H, m), 8.50 (1H, t, |
| | J = 6.0 Hz) |
| 1084 | 0.21-0.27 (2H, m), 0.38-0.44 (2H, m), 0.99-1.09 (1H, m), 2.44-2.49 (2H, m), 2.51 (3H, |
| | s), 2.63-2.71 (2H, m), 2.76-2.82 (2H, m), 3.11 (2H, t, J = 6.4 Hz), 3.15-3.22 (4H, m), 3.52 |
| | -3.58 (2H, m), 4.01 (2H, t, J = 6.6 Hz), 5.75-5.80 (1H, m), 5.84 (1H, q, J = 4.7 Hz), 7.05 |
| | (1H, s), 7.11 (2H, s), 7.47-7.55 (2H, m), 7.60-7.65 (1H, m), 7.76-7.78 (1H, m), 8.50 (1H, |
| | t, J = 6.0 Hz) |
| 1085 | 1.08 (3H, t, J = 7.1 Hz), 1.67 (4H, tq, J = 12.9, 8.2, 6.6 Hz), 2.46 (2H, d, J = 7.1 Hz), 2.63 (2H, |
| | t, J = 5.8 Hz), 2.79 (2H, t, J = 5.8 Hz), 3.18 (2H, t, J = 6.6 Hz), 3.35-3.41 (2H, m), 3.51 (2H, |
| | s), 3.87 (2H, dt, J = 11.5, 3.4 Hz), 4.02 (3H, t, J = 6.5 Hz), 7.05 (1H, d, J = 1.9 Hz), 7.10 (2H, |
| | d, J = 1.9 Hz), 7.46-7.55 (2H, m), 7.62 (1H, dt, J = 7.0, 2.0 Hz), 7.75-7.79 (1H, m), 8.33 |
| | (1H, d, J = 8.3 Hz) |
| 1086 | 1.07 (3H, t, J = 7.1 Hz), 2.15 (3H, s), 2.46 (2H, d, J = 7.1 Hz), 2.63 (2H, t, J = 5.8 Hz), 2.78 |
| | (2H, t, J = 5.8 Hz), 3.19 (2H, t, J = 6.6 Hz), 3.50 (2H, s), 4.01 (2H, t, J = 6.6 Hz), 4.35 (2H, d, |
| | J = 6.0 Hz), 5.90 (1H, d, J = 0.8 Hz), 7.04 (1H, t, J = 1.3 Hz), 7.10 (2H, d, J = 1.4 Hz), 7.46- |
| | 7.53 (2H, m), 7.61 (1H, dt, J = 6.7, 2.1 Hz), 7.77 (1H, dt, J = 2.4, 1.3 Hz), 8.69 (1H, t, J = 6.0 |
| | Hz), 12.18 (1H, s) |
| 1087 | 0.19-0.28 (2H, m), 0.35-0.46 (2H, m), 0.97-1.11 (1H, m), 1.60-1.71 (2H, m), 2.44- |
| | 2.49 (2H, m), 2.62-2.68 (2H, m), 2.78 (2H, t, J = 5.8 Hz), 3.11 (2H, t, J = 6.4 Hz), 3.19 (2H, |
| | t, J = 6.6 Hz), 3.46 (2H, t, J = 6.2 Hz), 3.47-3.54 (2H, m), 4.02 (2H, t, J = 6.6 Hz), 4.48 (1H, |
| | s), 7.03-7.09 (1H, m), 7.06-7.17 (2H, m), 7.46-7.57 (2H, m), 7.57-7.67 (1H, m), 7.75- |
| | 7.80 (1H, m), 8.52 (1H, t, J = 6.0 Hz) |
| 1088 | (CDCl3) 2.71 (2H, t, J = 5.3 Hz), 2.78-2.92 (5H, m), 3.38 (2H, t, J = 6.7 Hz), 3.66-3.71 (4H, |
| | m), 3.90 (3H, s), 4.08 (2H, t, J = 6.7 Hz), 4.68 (2H, d, J = 5.9 Hz), 6.25 (1H, d, J = 1.9 Hz), |
| | 6.98 (1H, d, J = 2.2 Hz), 7.06-7.15 (2H, m), 7.18 (1H, t, J = 5.9 Hz), 7.31-7.39 (2H, m), |
| | 7.41 (1H, d, J = 1.9 Hz), 7.45 (1H, dt, J = 7.6, 1.7 Hz), 7.56 (1H, t, J = 2.0 Hz) |
| 1089 | 2.56 (2H, t, J = 6.2 Hz), 2.67-2.73 (2H, m), 2.79 (2H, t, J = 5.8 Hz), 3.21 (2H, t, J = 6.6 Hz), |
| | 3.54-3.61 (4H, m), 4.03 (2H, t, J = 6.6 Hz), 4.46 (3H, q, J = 4.2, 3.0 Hz), 6.18 (1H, s), 7.05 |
| | (1H, d, J = 1.7 Hz), 7.11 (2H, d, J = 1.4 Hz), 7.50-7.54 (2H, m), 7.63 (2H, dt, J = 6.6, 2.2 Hz), |
| | 7.79 (1H, t, J = 1.6 Hz), 8.73 (1H, s), 12.58 (1H, s) |
| 1090 | 1.07 (3H, t, J = 7.2 Hz), 1.86-2.01 (2H, m), 2.13 (2H, dtd, J = 12.4, 8.0, 6.5 Hz), 2.60-2.67 |
| | (2H, m), 2.78 (2H, t, J = 5.8 Hz), 3.15-3.20 (2H, m), 3.50 (2H, qqd, J = 1.8, 0.8 Hz), 3.56- |
| | 3.60 (1H, m), 3.70 (1H, td, J = 8.1, 6.0 Hz), 3.84 (2H, ddt, J = 7.8, 6.3, 3.7 Hz), 4.01 (2H, t, |
| | J = 6.6 Hz), 4.40-4.53 (1H, m), 7.04 (1H, s), 7.09 (2H, d, J = 1.7 Hz), 7.46-7.54 (2H, m), 7.61 |
| | (1H, dt, J = 6.9, 2.1 Hz), 7.77 (1H, td, J = 1.9, 0.6 Hz), 8.49 (1H, d, J = 7.0 Hz) |
| 1091 | 1.09 (3H, t, J = 7.1 Hz), 1.97 (1H, dddd, J = 12.5, 7.7, 6.1, 4.9 Hz), 2.15 (1H, dtd, J = 12.5, |
| | 8.0, 6.5 Hz), 2.47 (2H, d, J = 7.1 Hz), 2.65 (2H, q, J = 7.0, 6.0 Hz), 2.80 (2H, t, J = 5.9 Hz), |
| | 3.19 (2H, t, J = 6.6 Hz), 3.52 (2H, s), 3.59 (1H, dd, J = 8.8, 4.9 Hz), 3.72 (1H, td, J = 8.1, 6.0 |
| | Hz), 3.86 (2H, ddt, J = 7.9, 6.4, 3.6 Hz), 4.03 (2H, t, J = 6.6 Hz), 4.42-4.55 (1H, m), 7.04- |
| | 7.07 (1H, m), 7.11 (2H, d, J = 1.6 Hz), 7.48-7.56 (2H, m), 7.63 (1H, dt, J = 6.9, 2.0 Hz), 7.79 |
| | (1H, td, J = 2.0, 0.6 Hz), 8.51 (1H, d, J = 7.0 Hz) |
| 1092 | 0.96 (3H, s), 1.05-1.12 (6H, m), 2.47 (1H, d, J = 7.2 Hz), 2.65 (2H, q, J = 6.0 Hz), 2.80 (2H, |
| | t, J = 5.9 Hz), 3.14-3.23 (2H, m), 3.45-3.56 (4H, m), 3.72 (1H, dd, J = 8.9, 6.5 Hz), 3.97- |
| | 4.09 (4H, m), 4.34 (1H, ddd, J = 9.0, 7.8, 6.5 Hz), 7.04-7.07 (1H, m), 7.11 (2H, d, J = 1.9 |
| | Hz), 7.49-7.56 (2H, m), 7.64 (1H, dt, J = 6.8, 2.1 Hz), 7.79 (1H, td, J = 1.9, 0.7 Hz), 8.24 |
| | (1H, d, J = 9.0 Hz) |
| 1093 | 1.07 (3H, t, J = 7.1 Hz), 1.92-1.98 (2H, m), 2.46 (2H, d, J = 7.2 Hz), 2.57-2.66 (3H, m), |
| | 2.78 (2H, t, J = 5.9 Hz), 3.18 (2H, t, J = 6.6 Hz), 3.50 (2H, s), 3.60-3.64 (2H, m), 3.84 (2H, d, |
| | J = 8.4 Hz), 4.01 (2H, t, J = 6.6 Hz), 7.03-7.06 (1H, m), 7.10 (2H, d, J = 1.8 Hz), 7.49-7.54 |
| | (2H, m), 7.60 (1H, dt, J = 6.9, 2.1 Hz), 7.75 (1H, td, J = 1.9, 0.6 Hz), 8.53 (1H, d, J = 4.5 Hz) |
| 1094 | 0.19-0.28 (2H, m), 0.35-0.46 (2H, m), 0.97-1.12 (1H, m), 2.77-2.82 (4H, m), 3.12 (2H, |
| | t, J = 6.4 Hz), 3.19 (2H, t, J = 6.6 Hz), 3.43 (2H, s), 3.64 (3H, s), 3.67-3.69 (2H, m), 4.02 |
| | (2H, t, J = 6.6 Hz), 7.03 (1H, d, J = 1.7 Hz), 7.12 (2H, d, J = 2.0 Hz), 7.46-7.58 (2H, m), |
| | 7.58-7.66 (1H, m), 7.75-7.80 (1H, m), 8.51 (1H, t, J = 6.0 Hz) |
| 1095 | 0.19-0.27 (2H, m), 0.35-0.46 (2H, m), 0.96-1.11 (1H, m), 2.99 (2H, t, J = 6.0 Hz), 3.11 |
| | (2H, d, J = 6.9 Hz), 3.19 (2H, t, J = 6.6 Hz), 3.25-3.29 (2H, m), 3.77-3.82 (2H, m), 4.02 |
| | (2H, t, J = 6.6 Hz), 4.16 (2H, s), 7.15 (1H, d, J = 1.6 Hz), 7.22 (2H, d, J = 1.4 Hz), 7.46-7.56 |
| | (2H, m), 7.56-7.67 (1H, m), 7.73-7.79 (1H, m) |
| 1096 | 0.19-0.28 (2H, m), 0.36-0.46 (2H, m), 0.97-1.10 (1H, m), 1.11 (6H, s), 2.34-2.38 (2H, |
| | m), 2.77-2.81 (4H, m), 3.12 (2H, t, J = 6.4 Hz), 3.19 (2H, t, J = 6.6 Hz), 3.68 (2H, s), 4.01 |
| | (2H, t, J = 6.6 Hz), 4.16 (1H, s), 7.01 (1H, s), 7.10 (2H, s), 7.46-7.57 (2H, m), 7.57-7.66 |
| | (1H, m), 7.74-7.80 (1H, m), 8.50 (1H, t, J = 6.0 Hz) |
| 1097 | 0.20-0.28 (2H, m), 0.36-0.46 (2H, m), 0.97-1.12 (1H, m), 1.12-1.28 (1H, m), 1.55- |
| | 1.68 (1H, m), 1.83-1.99 (1H, m), 2.65-2.78 (4H, m), 2.81 (2H, t, J = 5.7 Hz), 3.12 (2H, t, J = |
| | 6.5 Hz), 3.19 (2H, t, J = 6.6 Hz), 3.59 (2H, s), 4.02 (2H, t, J = 6.6 Hz), 7.03-7.07 (1H, m), |
| | 7.12 (2H, d, J = 1.8 Hz), 7.46-7.58 (2H, m), 7.58-7.66 (1H, m), 7.75-7.80 (1H, m), 8.50 |
| | (1H, t, J = 6.0 Hz) |
| 1098 | 0.20-0.27 (2H, m), 0.39-0.45 (2H, m), 1.00-1.10 (1H, m), 2.46 (2H, t, J = 6.5 Hz), 2.66 |
| | (2H, t, J = 5.8 Hz), 2.70 (2H, t, J = 6.4 Hz), 2.79 (2H, t, J = 5.8 Hz), 3.12 (2H, t, J = 6.5 Hz), |
| | 3.19 (2H, t, J = 6.8 Hz), 3.53 (2H, s), 4.02 (2H, t, J = 6.6 Hz), 7.05 (1H, s), 7.11 (2H, s), 7.47- |
| | 7.56 (2H, m), 7.62 (1H, dt, J = 6.9, 2.1 Hz), 7.76-7.79 (1H, m), 8.50 (1H, t, J = 6.0 Hz) |
| 1099 | 0.21-0.26 (2H, m), 0.39-0.45 (2H, m), 0.83-0.88 (2H, m), 0.89-0.94 (2H, m), 0.98- |
| | 1.04 (1H, m), 1.06 (3H, t, J = 7.1 Hz), 2.42-2.49 (4H, m), 3.12 (2H, t, J = 6.5 Hz), 3.19 (2H, |
| | t, J = 6.6 Hz), 3.62 (2H, s), 4.01 (2H, t, J = 6.6 Hz), 6.71 (1H, d, J = 8.5 Hz), 7.04 (1H, d, J = |
| | 2.2 Hz), 7.08 (1H, dd, J = 8.3, 2.3 Hz), 7.47-7.57 (2H, m), 7.62 (1H, dt, J = 6.8, 2.1 Hz), |
| | 7.75-7.80 (1H, m), 8.50 (1H, t, J = 6.0 Hz) |
| 1100 | 0.21-0.28 (2H, m), 0.39-0.46 (2H, m), 0.98-1.12 (1H, m), 2.75 (2H, t, J = 5.8 Hz), 2.85 |
| | (2H, t, J = 5.8 Hz), 3.12 (2H, t, J = 6.4 Hz), 3.20 (2H, t, J = 6.6 Hz), 3.65 (2H, s), 3.91 (2H, s), |
| | 4.03 (2H, t, J = 6.6 Hz), 7.10 (1H, s), 7.15 (2H, d, J = 1.3 Hz), 7.48-7.55 (2H, m), 7.62 (1H, |
| | dt, J = 6.7, 2.2 Hz), 7.77-7.80 (1H, m), 8.50 (1H, t, J = 6.0 Hz) |
| 1101 | 0.21-0.26 (2H, m), 0.39-0.45 (2H, m), 0.99-1.06 (1H, m), 1.07 (3H, d, J = 6.2 Hz), 2.29- |
| | 2.37 (1H, m), 2.41 (1H, dd, J = 12.3, 6.8 Hz), 2.71 (2H, t, J = 5.6 Hz), 2.78 (2H, t, J = 5.7 Hz), |
| | 3.12 (2H, t, J = 6.4 Hz), 3.19 (2H, t, J = 6.6 Hz), 3.58 (2H, s), 3.79-3.90 (1H, m), 4.02 (2H, t, |
| | J = 6.6 Hz), 4.37 (1H, d, J = 4.0 Hz), 7.03 (1H, d, J = 1.8 Hz), 7.10 (2H, d, J = 1.4 Hz), 7.47- |
| | 7.57 (2H, m), 7.62 (1H, dt, J = 6.7, 2.2 Hz), 7.76-7.79 (1H, m), 8.50 (1H, t, J = 6.0 Hz) |
| 1102 | 0.18-0.28 (2H, m), 0.34-0.48 (2H, m), 0.96-1.13 (1H, m), 1.13-1.30 (2H, m), 1.41- |
| | 1.63 (4H, m), 1.70 (2H, dq, J = 11.3, 6.3 Hz), 2.17 (1H, quintet, J = 7.5 Hz), 2.34 (2H, d, J = |
| | 7.6 Hz), 2.64 (2H, t, J = 5.7 Hz), 2.78 (2H, t, J = 5.7 Hz), 3.12 (2H, t, J = 6.5 Hz), 3.19 (2H, t, |
| | J = 6.7 Hz), 3.52 (2H, s), 4.02 (2H, t, J = 6.7 Hz), 7.04 (1H, s), 7.10 (2H, s), 7.45-7.57 (2H, m), |
| | 7.62 (1H, dt, J = 6.8, 2.1 Hz), 7.77 (1H, dt, J = 2.3, 1.3 Hz), 8.49 (1H, t, J = 6.0 Hz) |
| 1103 | 0.16-0.30 (2H, m), 0.36-0.51 (2H, m), 0.97-1.16 (1H, m), 1.67 (2H, m), 1.77-1.94 (2H, |
| | m), 1.96-2.15 (2H, m), 2.78 (3H, m), 3.12 (2H, t, J = 6.4 Hz), 3.19 (2H, t, J = 6.6 Hz), 3.38 |
| | (2H, m), 4.02 (2H, t, J = 6.6 Hz), 7.07 (1H, s), 7.12 (2H, s), 7.45-7.58 (2H, m), 7.62 (1H, dt, |
| | J = 6.8, 2.1 Hz), 7.77 (1H, t, J = 1.9 Hz), 8.50 (1H, t, J = 6.0 Hz) |
| 1104 | 0.12-0.30 (2H, m), 0.36-0.52 (2H, m), 1.04 (1H, ddd, J = 12.8, 7.9, 5.4 Hz), 2.64-2.72 |
| | (2H, m), 2.79 (2H, d, J = 5.3 Hz), 3.12 (2H, t, J = 6.4 Hz), 3.18 (2H, t, J = 6.6 Hz), 3.52 (2H, s), |
| | 3.67 (2H, s), 4.00 (2H, t, J = 6.7 Hz), 6.19 (1H, d, J = 1.5 Hz), 7.02 (1H, s), 7.11 (2H, s), 7.45- |
| | 7.57 (2H, m), 7.62 (1H, dt, J = 6.7, 2.2 Hz), 7.70-7.93 (1H, m), 8.50 (1H, t, J = 6.0 Hz), |
| | 12.37-12.78 (1H, m) |
| 1105 | 2.52-2.58 (2H, m), 2.66-2.72 (2H, m), 2.74-2.81 (3H, m), 3.15-3.22 (2H, m), 3.54- |
| | 3.61 (4H, m), 4.02 (2H, t, J = 6.7 Hz), 4.28 (1H, d, J = 6.1 Hz), 4.45 (1H, t, J = 5.3 Hz), 6.48 |
| | (1H, dd, J = 1.8, 0.9 Hz), 7.00-7.05 (1H, m), 7.11 (2H, dd, J = 7.8, 2.1 Hz), 7.49-7.55 (2H, |
| | m), 7.55-7.59 (2H, m), 7.62 (1H, dt, J = 6.8, 2.1 Hz), 7.75-7.79 (1H, m), 8.81 (1H, s) |
| 1106 | 1.57-1.66 (1H, m), 1.87-1.98 (2H, m), 2.71 (2H, d, J = 5.5 Hz), 2.79 (3H, d, J = 5.9 Hz), |
| | 3.16-3.27 (5H, m), 3.48 (1H, dd, J = 8.6, 5.2 Hz), 3.57-3.61 (4H, m), 3.64-3.70 (2H, m), |
| | 3.74 (1H, td, J = 8.0, 5.7 Hz), 4.03 (2H, t, J = 6.6 Hz), 4.46 (1H, t, J = 5.4 Hz), 7.04 (1H, d, J = |
| | 1.7 Hz), 7.11 (2H, d, J = 1.6 Hz), 7.50-7.54 (2H, m), 7.62 (1H, dt, J = 6.7, 2.1 Hz), 7.78 (1H, |
| | td, J = 1.9, 0.7 Hz), 8.59 (1H, t, J = 6.1 Hz) |
| 1107 | 2.53 (3H, ddd, J = 6.3, 4.3, 2.0 Hz), 2.66-2.69 (3H, m), 2.72-2.79 (4H, m), 3.19 (2H, t, J = |
| | 6.7 Hz), 4.01 (2H, t, J = 6.7 Hz), 4.44 (3H, d, J = 6.1 Hz), 7.01-7.03 (1H, m), 7.08-7.13 (3H, |
| | m), 7.21-7.27 (1H, m), 7.31-7.33 (3H, m), 7.49-7.52 (2H, m), 7.62 (1H, dt, J = 6.7, 2.1 |
| | Hz), 7.76-7.78 (1H, m), 9.01 (1H, t, J = 6.3 Hz) |
| 1108 | 1.97 (1H, dddd, J = 12.5, 7.8, 6.0, 4.9 Hz), 2.15 (1H, dtd, J = 12.5, 8.0, 6.5 Hz), 2.56 (2H, t, |
| | J = 6.2 Hz), 2.67-2.73 (2H, m), 2.79 (2H, d, J = 7.2 Hz), 3.17-3.21 (2H, m), 3.55-3.61 (5H, |
| | m), 3.68-3.75 (1H, m), 3.83-3.89 (2H, m), 4.02 (2H, t, J = 6.6 Hz), 4.48 (2H, dt, J = 10.9, |
| | 6.6 Hz), 7.04 (1H, d, J = 1.7 Hz), 7.11 (2H, d, J = 1.7 Hz), 7.50-7.53 (2H, m), 7.63 (1H, dt, |
| | J = 6.9, 2.1 Hz), 7.79 (1H, td, J = 1.9, 0.7 Hz), 8.51 (1H, d, J = 7.0 Hz) |
| 1109 | 2.52-2.57 (2H, m), 2.68 (2H, dd, J = 7.6, 4.2 Hz), 2.77 (2H, d, J = 5.7 Hz), 3.19 (2H, t, J = |
| | 6.7 Hz), 3.53-3.59 (4H, m), 4.01 (2H, t, J = 6.7 Hz), 4.41-4.46 (3H, m), 6.23-6.27 (1H, |
| | m), 6.38 (1H, dd, J = 3.2, 1.8 Hz), 7.03 (1H, t, J = 1.3 Hz), 7.09 (2H, d, J = 1.4 Hz), 7.48-7.52 |
| | (2H, m), 7.55-7.58 (1H, m), 7.60-7.64 (1H, m), 7.77 (1H, td, J = 1.9, 0.7 Hz), 8.88 (1H, t, |
| | J = 6.1 Hz) |
| 1110 | 2.33-2.36 (3H, m), 2.51-2.56 (2H, m), 2.66-2.71 (2H, m), 2.75-2.80 (2H, m), 3.16- |
| | 3.20 (2H, m), 3.53-3.60 (4H, m), 4.01 (2H, t, J = 6.6 Hz), 4.42-4.49 (3H, m), 7.01-7.03 |
| | (1H, m), 7.09 (2H, d, J = 1.8 Hz), 7.18 (1H, dt, J = 5.1, 0.8 Hz), 7.48-7.52 (2H, m), 7.61 (1H, |
| | dt, J = 6.9, 2.1 Hz), 7.77 (1H, td, J = 1.9, 0.6 Hz), 8.31 (1H, d, J = 4.9 Hz), 8.42 (1H, s), 8.97 |
| | (1H, t, J = 6.0 Hz) |
| 1111 | 2.53 (3H, s), 2.56 (2H, d, J = 6.3 Hz), 2.66-2.72 (2H, m), 2.78 (2H, d, J = 5.7 Hz), 3.19 (2H, |
| | t, J = 6.7 Hz), 3.54-3.60 (4H, m), 4.02 (2H, t, J = 6.6 Hz), 4.46 (3H, dt, J = 5.4, 2.3Hz), 7.02- |
| | 7.06 (1H, m), 7.11 (2H, d, J = 1.6 Hz), 7.20 (1H, dd, J = 7.7, 4.8 Hz), 7.49-7.55 (2H, m), |
| | 7.59-7.66 (2H, m), 7.79 (1H, td, J = 1.8, 0.7 Hz), 8.32 (1H, dd, J = 4.8, 1.7 Hz), 9.03 (1H, t, |
| | J = 6.1 Hz) |
| 1112 | 1.07 (3H, s), 1.23 (3H, s), 2.07-2.14 (1H, m), 2.22 (1H, ddt, J = 11.2, 5.8, 3.3 Hz), 2.55 (2H, |
| | t, J = 6.2 Hz), 2.67-2.72 (2H, m), 2.75-2.82 (2H, m), 3.15-3.22 (2H, m), 3.55-3.61 (4H, |
| | m), 3.66-3.73 (1H, m), 3.84 (1H, td, J = 8.5, 4.7 Hz), 4.03 (2H, dp, J = 9.8, 6.1 Hz), 4.31 |
| | (1H, td, J = 8.5, 7.3 Hz), 4.45 (1H, t, J = 5.4 Hz), 7.02-7.05 (1H, m), 7.10 (2H, d, J = 1.8 Hz), |
| | 7.50-7.53 (2H, m), 7.63 (1H, dt, J = 6.8, 2.1 Hz), 7.79 (1H, td, J = 1.9, 0.7 Hz), 8.24 (1H, d, |
| | J = 8.8 Hz) |
| 1113 | 0.19-0.33 (2H, m), 0.35-0.47 (2H, m), 0.99 (3H, d, J = 6.7 Hz), 1.02-1.10 (1H, m), 2.50- |
| | 2.58 (2H, m), 2.69-2.83 (4H, m), 3.12 (2H, t, J = 6.4 Hz), 3.19 (2H, t, J = 6.6 Hz), 3.48-3.62 |
| | (1H, m), 3.68 (2H, d, J = 3.0 Hz), 4.01 (2H, t, J = 6.7 Hz), 4.34 (1H, t, J = 5.2 Hz), 7.04 (1H, s), |
| | 7.09 (2H, s), 7.47-7.56 (2H, m), 7.62 (1H, dt, J = 6.8, 2.1 Hz), 7.77 (1H, t, J = 2.0 Hz), 8.51 |
| | (1H, t, J = 6.0 Hz) |
| 1114 | 0.18-0.28 (2H, m), 0.38-0.47 (2H, m), 1.05 (1H, ddd, J = 12.8, 8.0, 4.9 Hz), 2.78 (1H, d, |
| | J = 5.7 Hz), 2.85 (1H, t, J = 5.8 Hz), 3.12 (2H, t, J = 6.4 Hz), 3.20 (2H, t, J = 6.5 Hz), 3.57 (1H, t, |
| | J = 5.8 Hz), 3.69 (1H, t, J = 6.0 Hz), 4.00-4.07 (2H, m), 4.16 (2H, t, J = 6.0 Hz), 4.50-4.77 |
| | (3H, m), 7.11-7.31 (3H, m), 7.41-7.58 (2H, m), 7.63 (1H, dt, J = 6.6, 2.2 Hz), 7.78 (1H, d, |
| | J = 1.9 Hz), 8.51 (1H, t, J = 6.0 Hz) |
| 1115 | 0.15-0.32 (2H, m), 0.36-0.50 (2H, m), 0.97-1.13 (1H, m), 1.71-1.89 (1H, m), 2.06 (1H, |
| | dd, J = 11.1, 3.8 Hz), 2.54-2.77 (2H, m), 2.78 (2H, t, J = 5.5 Hz), 3.00-3.09 (1H, m), 3.12 |
| | (2H, t, J = 6.5 Hz), 3.19 (2H, t, J = 6.7 Hz), 3.48-3.64 (3H, m), 3.67 (1H, q, J = 8.0 Hz), 3.78- |
| | 3.91 (2H, m), 4.02 (2H, t, J = 6.6 Hz), 7.05 (1H, s), 7.11 (2H, d, J = 1.2 Hz), 7.45-7.57 (2H, |
| | m), 7.62 (1H, dt, J = 6.8, 2.1 Hz), 7.77 (1H, dt, J = 2.0, 1.2 Hz), 8.50 (1H, t, J = 6.0 Hz) |
| 1116 | 0.17-0.31 (2H, m), 0.36-0.50 (2H, m), 0.97-1.10 (1H, m), 1.45-1.62 (1H, m), 1.95 (1H, |
| | s), 2.42 (2H, m), 2.52-2.57 (1H, m), 2.67 (2H, br s), 2.79 (2H, s), 3.12 (2H, t, J = 6.5 Hz), |
| | 3.19 (2H, t, J = 6.6 Hz), 3.40 (1H, dd, J = 8.3, 5.9 Hz), 3.54 (2H, d, J = 6.5 Hz), 3.62 (1H, q, |
| | J = 7.6 Hz), 3.66-3.81 (2H, m), 4.02 (2H, t, J = 6.6 Hz), 7.05 (1H, s), 7.11 (2H, s), 7.45-7.57 |
| | (2H, m), 7.62 (1H, dt, J = 6.8, 2.1 Hz), 7.72-7.86 (1H, m), 8.50 (1H, t, J = 6.0 Hz) |
| 1117 | 0.15-0.28 (2H, m), 0.34-0.56 (2H, m), 0.98-1.15 (4H, m), 2.67 (2H, s), 2.80 (2H, s), 3.12 |
| | (2H, t, J = 6.5 Hz), 3.16 (2H, t, J = 6.5 Hz), 3.52 (2H, s), 4.29 (2H, t, J = 6.5 Hz), 7.40 (1H, s), |
| | 7.47-7.59 (2H, m), 7.64 (1H, dt, J = 7.0, 2.0 Hz), 7.81 (1H, d, J = 2.2 Hz), 8.23 (1H, s), 8.51 |
| | (1H, t, J = 6.1 Hz) 2H obscured by DMSO peak |
| 1118 | 0.21-0.25 (2H, m), 0.34-0.49 (2H, m), 1.03 (1H, m), 2.54 (2H, m), 2.74 (2H, br s), 2.80 |
| | (2H, br s), 3.12 (2H, t, J = 6.4 Hz), 3.16 (2H, t, J = 6.4 Hz), 3.58 (4H, br s), 4.29 (2H, t, J = 6.5 |
| | Hz), 4.48 (1H, br s), 7.39 (1H, s), 7.47-7.59 (2H, m), 7.64 (1H, dt, J = 6.9, 2.0 Hz), 7.81 (1H, |
| | d, J = 2.1 Hz), 8.23 (1H, s), 8.51 (1H, t, J = 6.2 Hz) |
| 1119 | 0.20-0.30 (2H, m), 0.38-0.48 (2H, m), 1.00-1.10 (1H, m), 1.39-1.68 (4H, m), 1.78 (1H, |
| | dt, J = 12.1, 7.3 Hz), 1.87 (1H, dd, J = 11.7, 6.9 Hz), 2.52-2.58 (1H, m), 2.62-2.69 (1H, m), |
| | 2.77 (2H, t, J = 5.8 Hz), 2.83-2.92 (1H, m), 3.12 (2H, t, J = 6.5 Hz), 3.19 (2H, t, J = 6.6 Hz), |
| | 3.64 (2H, s), 3.96-4.06 (3H, m), 4.63 (1H, d, J = 5.0 Hz), 7.04 (1H, s), 7.09 (2H, d, J = 1.3 |
| | Hz), 7.46-7.58 (2H, m), 7.62 (1H, dt, J = 6.7, 2.1 Hz), 7.75-7.80 (1H, m), 8.50 (1H, t, J = |
| | 6.0 Hz) |
| 1120 | 0.19-0.29 (2H, m), 0.37-0.46 (2H, m), 0.99-1.11 (1H, m), 1.68-1.79 (1H, m), 2.03- |
| | 2.23 (3H, m), 2.41 (1H, dd, J = 12.3, 6.6 Hz), 2.46-2.49 (1H, m), 2.65-2.77 (2H, m), 2.79 |
| | (2H, t, J = 6.2 Hz), 3.12 (2H, t, J = 6.4 Hz), 3.19 (2H, t, J = 6.6 Hz), 3.59 (2H, s), 3.78 (1H, |
| | quintet, J = 6.3 Hz), 4.02 (2H, t, J = 6.6 Hz), 7.04 (1H, s), 7.11 (2H, d, J = 1.7 Hz), 7.48-7.55 |
| | (2H, m), 7.60-7.64 (2H, m), 7.75-7.81 (1H, m), 8.50 (1H, t, J = 6.0 Hz) |
| 1121 | 0.24 (2H, dt, J = 6.1, 4.3 Hz), 0.39-0.46 (2H, m), 1.00-1.06 (1H, m), 1.08 (3H, t, J = 7.1 |
| | Hz), 2.45-2.48 (2H, m), 2.67 (2H, dt, J = 5.9, 1.8 Hz), 2.71 (2H, d, J = 5.4 Hz), 3.12 (2H, t, J = |
| | 6.4 Hz), 3.20 (2H, t, J = 6.6 Hz), 3.53 (2H, s), 4.04 (2H, t, J = 6.6 Hz), 6.96 (1H, d, J = 2.0 |
| | Hz), 7.06 (1H, dd, J = 10.7, 2.0 Hz), 7.48-7.57 (2H, m), 7.63 (1H, dt, J = 6.7, 2.2 Hz), 7.75- |
| | 7.81 (1H, m), 8.51 (1H, t, J = 6.0 Hz) |
| 1122 | 0.24 (2H, dt, J = 6.1, 4.3 Hz), 0.39-0.46 (2H, m), 0.99-1.11 (1H, m), 2.56 (2H, t, J = 6.2 |
| | Hz), 2.69-2.76 (4H, m), 3.12 (2H, t, J = 6.4 Hz), 3.20 (2H, t, J = 6.6 Hz), 3.53-3.64 (4H, m), |
| | 4.03 (2H, t, J = 6.6 Hz), 4.49 (1H, t, J = 5.4 Hz), 6.95 (1H, d, J = 2.0 Hz), 7.06 (1H, dd, J = |
| | 10.8, 2.0 Hz), 7.47-7.57 (2H, m), 7.58-7.67 (1H, m), 7.76-7.81 (1H, m), 8.51 (1H, t, J = |
| | 6.0 Hz) |
| 1123 | 0.20-0.28 (2H, m), 0.36-0.46 (2H, m), 0.99-1.09 (1H, m), 2.52-2.59 (2H, m), 2.68 (2H, |
| | t, J = 5.8 Hz), 2.80 (2H, t, J = 5.7 Hz), 3.12 (2H, t, J = 6.5 Hz), 3.21 (2H, t, J = 6.6 Hz), 3.53- |
| | 3.61 (4H, m), 3.96 (2H, t, J = 6.6 Hz), 4.47 (1H, t, J = 5.4 Hz), 7.03 (1H, d, J = 11.0 Hz), 7.14 |
| | (1H, d, J = 7.7 Hz), 7.49-7.56 (2H, m), 7.56-7.65 (1H, m), 7.74-7.80 (1H, m), 8.52 (1H, t, |
| | J = 6.0 Hz) |
| 1124 | 0.19-0.28 (2H, m), 0.37-0.46 (2H, m), 0.99-1.10 (1H, m), 1.67-1.79 (1H, m), 2.03- |
| | 2.16 (2H, m), 2.36-2.45 (1H, m), 2.45-2.49 (1H, m), 2.50-2.57 (1H, m), 2.63-2.81 (2H, |
| | m), 2.78-2.82 (2H, m), 3.12 (2H, t, J = 6.5 Hz), 3.19 (2H, t, J = 6.6 Hz), 3.59 (2H, s), 3.73- |
| | 3.81 (1H, m), 4.02 (2H, t, J = 6.6 Hz), 7.04 (1H, s), 7.11 (2H, s), 7.46-7.57 (2H, m), 7.57- |
| | 7.66 (2H, m), 7.75-7.80 (1H, m), 8.50 (1H, t, J = 6.0 Hz) |
| 1125 | 2.17 (3H, s), 2.69 (2H, s), 2.80 (2H, s), 3.21 (2H, t, J = 6.4 Hz), 3.35-3.42 (2H, m), 3.54- |
| | 3.59 (4H, m), 3.96 (2H, t, J = 6.2 Hz), 4.36 (2H, s), 4.47 (1H, s), 5.91 (1H, s), 7.03 (1H, d, J = |
| | 10.8 Hz), 7.15 (1H, d, J = 7.7 Hz), 7.46-7.56 (2H, m), 7.57-7.65 (1H, m), 7.77 (1H, s), 8.68 |
| | (1H, s), 12.21 (1H, s) |
| 1126 | 2.28 (3H, s), 2.55 (2H, t, J = 6.2 Hz), 2.70 (2H, d, J = 5.4 Hz), 2.78 (2H, t, J = 6.0 Hz), 3.20 |
| | (2H, q, J = 6.7, 6.2 Hz), 3.57 (3H, t, J = 2.7 Hz), 3.66 (1H, s), 4.02 (2H, s), 4.44 (3H, dd, J = |
| | 7.5, 5.8 Hz), 7.03 (1H, d, J = 1.7 Hz), 7.10 (1H, d, J = 1.6 Hz), 7.49-7.57 (3H, m), 7.60-7.65 |
| | (1H, m), 7.76-7.79 (1H, m), 8.26-8.33 (2H, m), 8.36 (1H, d, J = 2.1 Hz), 9.06 (1H, t, J = 6.3 |
| | Hz) |
| 1127 | 0.61 (2H, s), 0.84 (2H, d, J = 6.6 Hz), 1.82 (1H, d, J = 8.5 Hz), 2.54 (2H, t, J = 6.2 Hz), 2.66- |
| | 2.72 (3H, m), 2.76-2.81 (2H, m), 3.17-3.20 (2H, m), 3.55-3.59 (3H, m), 4.02 (2H, t, J = |
| | 6.6 Hz), 4.35 (2H, s), 4.44 (1H, t, J = 5.4 Hz), 5.82 (1H, s), 7.03 (1H, s), 7.10 (2H, d, J = 1.5 |
| | Hz), 7.46-7.53 (2H, m), 7.59-7.63 (1H, m), 7.77 (1H, td, J = 1.9, 0.7 Hz), 8.64 (1H, s), |
| | 12.26 (1H, s) |
| 1128 | 2.31 (3H, s), 2.38 (3H, s), 2.54 (2H, t, J = 6.2 Hz), 2.65-2.73 (2H, m), 2.75-2.82 (2H, m), |
| | 3.18 (2H, t, J = 6.6 Hz), 3.51-3.61 (4H, m), 4.01 (2H, t, J = 6.6 Hz), 4.43 (2H, d, J = 5.9 Hz), |
| | 4.46 (1H, t, J = 5.4 Hz), 7.03 (1H, s), 7.04 (1H, s), 7.07-7.13 (2H, m), 7.47-7.54 (2H, m), |
| | 7.62 (1H, dt, J = 6.9, 2.0 Hz), 7.73-7.82 (1H, m), 8.30 (1H, s), 8.92 (1H, t, J = 6.0 Hz) |
| 1129 | 2.31 (3H, s), 2.35 (3H, s), 2.57 (2H, t, J = 5.9 Hz), 2.79 (2H, t, J = 4.6 Hz), 3.18 (2H, t, J = 6.6 |
| | Hz), 3.44 (2H, s), 4.01 (2H, t, J = 6.6 Hz), 4.47 (2H, d, J = 6.0 Hz), 7.02 (1H, d, J = 1.9 Hz), |
| | 7.10 (2H, t, J = 1.6 Hz), 7.18 (1H, d, J = 4.9 Hz), 7.47-7.53 (2H, m), 7.61 (1H, dt, J = 6.9, 2.1 |
| | Hz), 7.77 (1H, t, J = 1.7 Hz), 8.31 (1H, d, J = 4.9 Hz), 8.42 (1H, s), 8.97 (1H, t, J = 6.0 Hz) |
| 1130 | 2.53-2.57 (2H, m), 2.67-2.72 (2H, m), 2.79 (2H, d, J = 5.9 Hz), 3.19 (2H, t, J = 6.6 Hz), |
| | 3.55-3.61 (4H, m), 4.03 (2H, t, J = 6.6 Hz), 4.48 (1H, t, J = 5.4 Hz), 4.65 (2H, dd, J = 5.7, 1.8 |
| | Hz), 7.05 (1H, d, J = 1.5 Hz), 7.11 (2H, d, J = 1.3 Hz), 7.41 (1H, dt, J = 8.6, 4.4 Hz), 7.51- |
| | 7.55 (2H, m), 7.64 (1H, dt, J = 6.7, 2.2 Hz), 7.71 (1H, ddd, J = 10.3, 8.4, 1.3 Hz), 7.80 (1H, |
| | td, J = 1.8, 0.7 Hz), 8.40 (1H, dt, J = 4.7, 1.5 Hz), 8.84 (1H, t, J = 5.7 Hz) |
| 1131 | 1.54 (3H, d, J = 7.0 Hz), 2.52-2.58 (2H, m), 2.69 (2H, dd, J = 7.6, 4.6 Hz), 2.79 (2H, t, J = |
| | 5.5 Hz), 3.11-3.22 (3H, m), 3.58 (3H, d, J = 5.0 Hz), 4.00 (2H, t, J = 6.7 Hz), 4.48 (1H, t, J = |
| | 5.4 Hz), 5.18-5.26 (1H, m), 7.03 (1H, d, J = 1.8 Hz), 7.10 (1H, d, J = 1.8 Hz), 7.37 (1H, ddd, |
| | J = 7.9, 4.8, 0.9 Hz), 7.49-7.56 (2H, m), 7.64 (1H, dt, J = 7.1, 2.0 Hz), 7.77-7.88 (2H, m), |
| | 8.46 (1H, dd, J = 4.8, 1.7 Hz), 8.64 (1H, d, J = 2.1 Hz), 8.95 (1H, dd, J = 8.5, 3.3 Hz) (one |
| | proton not seen) |
| 1132 | 2.54 (2H, t, J = 6.2 Hz), 2.65-2.71 (2H, m), 2.77 (2H, d, J = 5.7 Hz), 3.19 (2H, t, J = 6.6 Hz), |
| | 3.53-3.59 (4H, m), 3.77 (3H, s), 4.01 (2H, t, J = 6.6 Hz), 4.37 (2H, d, J = 6.0 Hz), 4.44 (1H, t, |
| | J = 5.4 Hz), 6.12 (1H, d, J = 2.2 Hz), 7.03 (1H, d, J = 1.7 Hz), 7.09 (2H, d, J = 1.4 Hz), 7.48- |
| | 7.51 (2H, m), 7.56 (1H, d, J = 2.2 Hz), 7.61 (1H, dt, J = 6.6, 2.2 Hz), 7.75-7.80 (1H, m), 8.71 |
| | (1H, t, J = 6.1 Hz) |
| 1133 | 2.55 (2H, t, J = 6.2 Hz), 2.69 (2H, t, J = 5.8 Hz), 2.76-2.82 (2H, m), 3.19 (2H, t, J = 6.6 Hz), |
| | 3.54-3.61 (4H, m), 3.84 (3H, s), 4.02 (2H, t, J = 6.7 Hz), 4.48 (3H, dd, J = 7.6, 5.7 Hz), 6.16 |
| | (1H, d, J = 1.8 Hz), 7.02-7.05 (1H, m), 7.10 (2H, d, J = 1.7 Hz), 7.30 (1H, d, J = 1.8 Hz), |
| | 7.49-7.55 (2H, m), 7.62 (1H, dt, J = 7.0, 2.0 Hz), 7.78 (1H, td, J = 1.9, 0.6 Hz), 9.01 (1H, t, |
| | J = 6.1 Hz) |
| 1134 | 2.36 (3H, s), 2.45-2.57 (2H, m), 2.68-2.73 (1H, m), 2.79-2.84 (2H, m), 3.21 (2H, t, J = |
| | 6.6 Hz), 3.26-3.37 (2H, m), 3.56-3.60 (4H, m), 3.96 (2H, t, J = 6.6 Hz), 4.48 (2H, d, J = 6.0 |
| | Hz), 7.05 (1H, d, J = 11.0 Hz), 7.12-7.22 (2H, m), 7.46-7.57 (2H, m), 7.57-7.65 (1H, m), |
| | 7.74-7.80 (1H, m), 8.32 (1H, d, J = 4.9 Hz), 8.44 (1H, s), 9.01 (1H, t, J = 6.0 Hz) |
| 1135 | 2.32 (3H, d, J = 3.1 Hz), 2.58 (2H, t, J = 5.8 Hz), 2.77-2.83 (2H, m), 3.03 (1H, d, J = 1.6 Hz), |
| | 3.20 (2H, q, J = 6.7, 6.0 Hz), 3.45 (2H, d, J = 2.6 Hz), 3.83 (3H, s), 4.02 (2H, t, J = 6.7 Hz), |
| | 4.38 (2H, d, J = 6.2 Hz), 6.77-6.81 (1H, m), 7.03 (1H, d, J = 1.9 Hz), 7.11 (1H, q, J = 2.1 Hz), |
| | 7.50-7.54 (2H, m), 7.62 (1H, dt, J = 7.0, 2.0 Hz), 7.69 (1H, dt, J = 8.5, 2.0 Hz), 7.77 (1H, t, |
| | J = 2.1 Hz), 8.13 (1H, dd, J = 2.5, 0.9 Hz), 8.99-9.04 (1H, m) |
| 1136 | 2.33 (3H, s), 2.55 (2H, t, J = 6.2 Hz), 2.65-2.72 (2H, m), 2.78 (2H, d, J = 5.9 Hz), 3.22 (3H, |
| | q, J = 6.5 Hz), 3.57 (4H, q, J = 5.7, 4.1 Hz), 4.03 (2H, t, J = 6.6 Hz), 4.59 (2H, d, J = 5.2 Hz), |
| | 7.04 (1H, t, J = 1.3 Hz), 7.11 (1H, d, J = 1.3 Hz), 7.23 (1H, dd, J = 7.5, 5.0 Hz), 7.49-7.57 |
| | (3H, m), 7.58-7.66 (2H, m), 7.79 (1H, dt, J = 2.3, 1.2 Hz), 8.36-8.40 (1H, m), 8.70-8.76 |
| | (1H, m) |
| 1137 | 2.54 (2H, t, J = 6.2 Hz), 2.69 (2H, t, J = 5.7 Hz), 2.78 (2H, t, J = 5.8 Hz), 3.19 (2H, t, J = 6.6 |
| | Hz), 3.52-3.61 (4H, m), 4.01 (2H, t, J = 6.6 Hz), 4.44 (1H, t, J = 5.4 Hz), 4.50 (2H, d, J = 6.2 |
| | Hz), 7.03 (1H, d, J = 1.8 Hz), 7.09 (2H, d, J = 1.8 Hz), 7.47-7.55 (2H, m), 7.63 (2H, ddt, J = |
| | 9.2, 7.0, 2.0 Hz), 7.78 (1H, dt, J = 2.3, 1.2 Hz), 8.43-8.49 (2H, m), 9.13 (1H, t, J = 6.2 Hz) |
| 1138 | 2.52-2.58 (2H, m), 2.66-2.72 (2H, m), 2.78 (2H, d, J = 5.9 Hz), 3.19 (2H, t, J = 6.6 Hz), |
| | 3.55-3.61 (4H, m), 3.83 (3H, s), 4.02 (2H, t, J = 6.6 Hz), 4.38 (2H, d, J = 6.2 Hz), 4.45 (1H, t, |
| | J = 5.4 Hz), 6.79 (1H, dd, J = 8.5, 0.8 Hz), 7.02-7.05 (1H, m), 7.10 (2H, d, J = 1.7 Hz), 7.48- |
| | 7.55 (2H, m), 7.62 (1H, dt, J = 7.0, 2.0 Hz), 7.69 (1H, dd, J = 8.5, 2.5 Hz), 7.77 (1H, td, J = |
| | 1.9, 0.6 Hz), 8.13 (1H, dd, J = 2.4, 0.8 Hz), 9.01 (1H, t, J = 6.2 Hz) |
| 1139 | 2.45 (3H, d, J = 1.8 Hz), 2.54 (2H, t, J = 6.2 Hz), 2.65-2.71 (2H, m), 2.78 (2H, t, J = 5.8 Hz), |
| | 3.13-3.24 (3H, m), 3.52-3.61 (4H, m), 4.02 (2H, t, J = 6.6 Hz), 4.44 (1H, q, J = 4.7, 3.9 Hz), |
| | 4.50 (2H, d, J = 6.2 Hz), 7.03 (1H, d, J = 1.5 Hz), 7.10 (2H, t, J = 1.2 Hz), 7.12 (1H, s), 7.49- |
| | 7.53 (2H, m), 7.61-7.66 (2H, m), 7.79 (1H, td, J = 2.2, 0.8 Hz), 9.01 (1H, t, J = 6.2 Hz) |
| 1140 | 1.09 (3H, t, J = 7.2 Hz), 2.36-2.38 (3H, m), 2.47 (2H, d, J = 7.2 Hz), 2.61-2.69 (2H, m), |
| | 2.80 (2H, t, J = 5.8 Hz), 3.20 (3H, t, J = 6.6 Hz), 3.52 (2H, s), 4.03 (2H, t, J = 6.7 Hz), 4.48 |
| | (2H, d, J = 6.0 Hz), 7.06 (1H, d, J = 1.8 Hz), 7.11 (1H, d, J = 1.8 Hz), 7.20 (1H, dt, J = 5.0, 0.8 |
| | Hz), 7.49-7.56 (2H, m), 7.63 (1H, dt, J = 7.0, 2.0 Hz), 7.79 (1H, td, J = 2.0, 0.6 Hz), 8.33 |
| | (1H, d, J = 4.9 Hz), 8.44 (1H, s), 8.99 (1H, s) |
| 1141 | 2.55 (2H, t, J = 6.2 Hz), 2.70 (2H, t, J = 5.7 Hz), 2.79 (2H, t, J = 5.7 Hz), 3.20 (2H, t, J = 6.6 |
| | Hz), 3.53-3.62 (4H, m), 3.82 (3H, s), 4.02 (2H, t, J = 6.6 Hz), 4.47 (3H, d, J = 6.2 Hz), 7.04 |
| | (1H, d, J = 1.7 Hz), 7.11 (2H, d, J = 1.8 Hz), 7.34 (1H, dd, J = 2.9, 1.7 Hz), 7.47-7.58 (2H, |
| | m), 7.63 (1H, dt, J = 6.9, 2.1 Hz), 7.79 (1H, td, J = 1.9, 0.6 Hz), 8.18 (2H, dd, J = 9.3, 2.3 Hz), |
| | 9.09 (1H, t, J = 6.2 Hz) |
| 1142 | 1.32 (1H, ddt, J = 15.6, 7.6, 3.9 Hz), 1.57 (1H, tdd, J = 12.1, 7.7, 4.5 Hz), 1.96-2.07 (1H, |
| | m), 2.54 (2H, t, J = 6.2 Hz), 2.68 (2H, t, J = 5.8 Hz), 2.77 (2H, d, J = 5.8 Hz), 3.19 (3H, t, J = |
| | 6.7 Hz), 3.41-3.50 (1H, m), 3.54-3.59 (4H, m), 4.01 (2H, t, J = 6.6 Hz), 4.44 (1H, t, J = 5.4 |
| | Hz), 7.03 (1H, t, J = 1.3 Hz), 7.09 (2H, d, J = 1.5 Hz), 7.49-7.53 (2H, m), 7.61 (1H, dt, J = |
| | 6.9, 2.0 Hz), 7.76 (1H, td, J = 1.9, 0.6 Hz), 8.69 (1H, t, J = 5.9 Hz) |
| 1143 | 0.21-0.28 (2H, m), 0.38-0.46 (2H, m), 0.83-0.90 (2H, m), 0.90-0.95 (2H, m), 0.99- |
| | 1.11 (1H, m), 2.53-2.62 (4H, m), 3.12 (2H, t, J = 6.5 Hz), 3.19 (2H, t, J = 6.6 Hz), 3.56 (2H, |
| | q, J = 6.0 Hz), 3.70 (2H, s br), 4.01 (2H, t, J = 6.6 Hz), 4.46 (1H, s br), 6.72 (1H, d, J = 8.5 |
| | Hz), 7.02 (1H, d, J = 2.2 Hz), 7.08 (1H, dd, J = 8.4, 2.2 Hz), 7.47-7.56 (2H, m), 7.57-7.66 |
| | (1H, m), 7.75-7.79 (1H, m), 8.49 (1H, t, J = 6.0 Hz) |
| 1144 | 0.86 (2H, t, J = 4.1 Hz), 0.91 (2H, t, J = 4.3 Hz), 2.36 (3H, s), 2.52-2.58 (4H, m), 3.19 (2H, t, |
| | J = 6.6 Hz), 3.56 (2H, q, J = 6.0 Hz), 3.69 (2H, s), 4.01 (2H, t, J = 6.6 Hz), 4.44 (1H, t, J = 5.4 |
| | Hz), 4.47 (2H, d, J = 6.0 Hz), 6.71 (1H, d, J = 8.5 Hz), 7.02 (1H, d, J = 2.2 Hz), 7.08 (1H, dd, |
| | J = 8.3, 2.2 Hz), 7.18 (1H, d, J = 4.9 Hz), 7.45-7.58 (2H, m), 7.62 (1H, dt, J = 6.8, 2.1 Hz), |
| | 7.75-7.80 (1H, m), 8.32 (1H, d, J = 4.9 Hz), 8.43 (1H, s), 8.98 (1H, t, J = 6.0 Hz) |
| 1145 | 1.53 (3H, d, J = 7.1 Hz), 2.54 (2H, t, J = 6.3 Hz), 2.66-2.72 (2H, m), 2.78 (2H, t, J = 5.9 Hz), |
| | 3.10-3.21 (3H, m), 3.57 (4H, d, J = 4.6 Hz), 4.00 (2H, t, J = 6.7 Hz), 4.45 (1H, t, J = 5.4 Hz), |
| | 5.22 (1H, quintet, J = 7.2 Hz), 7.02 (1H, d, J = 1.8 Hz), 7.09 (1H, d, J = 1.8 Hz), 7.36 (1H, |
| | ddd, J = 7.9, 4.8, 0.9 Hz), 7.50-7.55 (2H, m), 7.63 (1H, dt, J = 7.0, 2.0 Hz), 7.77-7.85 (2H, |
| | m), 8.45 (1H, dd, J = 4.8, 1.6 Hz), 8.63 (1H, d, J = 2.3 Hz), 8.91 (1H, d, J = 8.3 Hz) |
| 1146 | (CD3OD) 1.01-1.07 (4H, m), 1.92 (1H, s), 2.33-2.54 (2H, m), 2.77 (2H, t, J = 6.0 Hz), 2.89- |
| | 2.98 (4H, m), 3.56-3.62 (1H, m), 3.76-3.83 (3H, m), 4.10 (2H, t, J = 6.7 Hz), 4.54 (2H, s), |
| | 6.24 (1H, d, J = 2.4 Hz), 7.10 (1H, d, J = 2.0 Hz), 7.14-7.20 (2H, m), 7.23-7.38 (2H, m), |
| | 7.43-7.47 (2H, m), 7.57-7.61 (2H, m), 7.73 (1H, qd, J = 2.3, 1.0 Hz) |
| 1147 | 2.42 (3H, s), 2.54 (2H, t, J = 6.3 Hz), 2.68 (2H, t, J = 5.8 Hz), 2.78 (2H, t, J = 5.7 Hz), 3.18 |
| | (2H, t, J = 6.6 Hz), 3.57 (4H, d, J = 4.4 Hz), 4.01 (2H, t, J = 6.6 Hz), 4.40 (2H, d, J = 6.2 Hz), |
| | 4.47 (1H, t, J = 5.4 Hz), 7.01-7.04 (1H, m), 7.09 (2H, d, J = 1.8 Hz), 7.20 (1H, d, J = 8.0 Hz), |
| | 7.48-7.54 (2H, m), 7.59-7.64 (2H, m), 7.76-7.78 (1H, m), 8.41 (1H, dd, J = 2.3, 0.8 Hz), |
| | 9.06 (1H, t, J = 6.2 Hz) |
| 1148 | 1.23 (3H, d, J = 6.9 Hz), 2.36 (3H, s), 2.37-2.41 (1H, m), 2.52-2.57 (2H, m), 2.74-2.82 |
| | (1H, m), 2.89-2.97 (1H, m), 3.19 (2H, t, J = 6.6 Hz), 3.54-3.61 (4H, m), 4.02 (2H, t, J = 6.6 |
| | Hz), 4.44 (1H, s br), 4.47 (2H, d, J = 6.0 Hz), 7.02 (1H, d, J = 2.2 Hz), 7.13 (1H, dd, J = 8.2, |
| | 2.2 Hz), 7.19 (1H, d, J = 5.0 Hz), 7.24 (1H, d, J = 8.3 Hz), 7.46-7.56 (2H, m), 7.62 (1H, dt, |
| | J = 7.0, 2.0 Hz), 7.75-7.80 (1H, m), 8.32 (1H, d, J = 4.9 Hz), 8.43 (1H, s), 8.99 |
| | (1H, t, J = 6.0 Hz) |
| 1149 | 0.21-0.27 (2H, m), 0.38-0.45 (2H, m), 1.00-1.10 (1H, m), 2.97 (2H, t, J = 6.6 Hz), 3.03 |
| | (3H, s), 3.12 (2H, t, J = 6.4 Hz), 3.22 (2H, t, J = 6.6 Hz), 3.55 (2H, dd, J = 7.1, 6.2 Hz), 4.06 |
| | (2H, t, J = 6.6 Hz), 7.32 (1H, d, J = 8.1 Hz), 7.45 (1H, dd, J = 8.1, 2.3 Hz), 7.48-7.55 (2H, m), |
| | 7.61-7.68 (1H, m), 7.79-7.81 (1H, m), 7.83 (1H, d, J = 2.3 Hz), 8.51 (1H, t, J = 6.0 Hz) |
| 1150 | 0.62 (2H, s), 0.86 (2H, s), 1.09 (3H, t, J = 7.2 Hz), 1.84 (1H, s), 2.47 (2H, d, J = 7.1 Hz), 2.62- |
| | 2.68 (2H, m), 2.80 (2H, t, J = 5.8 Hz), 3.20 (2H, t, J = 6.6 Hz), 3.52 (2H, s), 4.03 (2H, t, J = |
| | 6.6 Hz), 4.36 (2H, s), 5.83 (1H, s), 7.06 (1H, s), 7.12 (2H, d, J = 1.6 Hz), 7.50-7.55 (2H, m), |
| | 7.63 (1H, dt, J = 6.8, 2.1 Hz), 7.79 (1H, dt, J = 2.3, 1.2 Hz), 8.68 (1H, s), 12.23 (1H, d, J = |
| | 47.5 Hz) |
| 1151 | 2.54 (1H, t, J = 6.2 Hz), 2.66-2.72 (2H, m), 2.75-2.81 (2H, m), 3.17-3.21 (2H, m), 3.54- |
| | 3.60 (4H, m), 4.02 (2H, t, J = 6.5 Hz), 4.47 (1H, t, J = 5.4 Hz), 4.55 (2H, d, J = 6.1 Hz), 7.04 |
| | (1H, s), 7.11 (2H, d, J = 1.4 Hz), 7.16 (1H, d, J = 0.9 Hz), 7.49-7.56 (3H, m), 7.64 (1H, dt, J = |
| | 6.8, 2.1 Hz), 7.79 (1H, td, J = 1.9, 0.7 Hz), 8.05 (1H, d, J = 0.8 Hz), 9.10 (1H, t, J = 6.0 Hz) |
| 1152 | 0.21-0.27 (2H, m), 0.39-0.45 (2H, m), 1.01-1.10 (1H, m), 1.23 (3H, d, J = 6.9 Hz), 2.39 |
| | (1H, dd, J = 11.4, 6.5 Hz), 2.52-2.59 (2H, m), 2.79 (1H, dd, J = 11.3, 5.0 Hz), 2.94 (1H, q, J = |
| | 6.3 Hz), 3.12 (2H, t, J = 6.5 Hz), 3.19 (2H, t, J = 6.6 Hz), 3.55-3.61 (4H, m), 4.02 (2H, t, J = |
| | 6.6 Hz), 4.45 (1H, t, J = 5.4 Hz), 7.02 (1H, d, J = 2.2 Hz), 7.13 (1H, dd, J = 8.2, 2.2 Hz), 7.24 |
| | (1H, d, J = 8.4 Hz), 7.47-7.57 (2H, m), 7.62 (1H, dt, J = 6.8, 2.1 Hz), 7.75-7.79 (1H, m), |
| | 8.49 (1H, t, J = 6.0 Hz) |
| 1153 | 0.21-0.27 (2H, m), 0.39-0.45 (2H, m), 1.00-1.05 (1H, m), 1.08 (3H, t, J = 7.1 Hz), 1.23 |
| | (3H, d, J = 6.9 Hz), 2.33 (1H, dd, J = 11.4, 6.2 Hz), 2.40-2.47 (2H, m), 2.70 (1H, dd, J = |
| | 11.4, 5.1 Hz), 2.93 (1H, heptet, J = 6.3 Hz), 3.12 (2H, t, J = 6.4 Hz), 3.19 (2H, t, J = 6.6 Hz), |
| | 3.49 (2H, s), 4.02 (2H, t, J = 6.6 Hz), 7.04 (1H, d, J = 2.2 Hz), 7.13 (1H, dd, J = 8.3, 2.2 Hz), |
| | 7.24 (1H, d, J = 8.3 Hz), 7.47-7.56 (2H, m), 7.62 (1H, dt, J = 6.9, 2.0 Hz), 7.75-7.80 (1H, |
| | m), 8.50 (1H, t, J = 6.0 Hz) |
| 1154 | 2.57 (2H, d, J = 6.1 Hz), 2.70 (2H, d, J = 11.9 Hz), 2.79 (2H, d, J = 5.8 Hz), 3.20 (2H, t, J = 6.6 |
| | Hz), 3.58 (4H, q, J = 6.1 Hz), 4.03 (2H, t, J = 6.6 Hz), 4.49 (1H, s), 4.58 (2H, d, J = 5.9 Hz), |
| | 7.05 (1H, s), 7.11 (2H, d, J = 1.4 Hz), 7.50-7.58 (3H, m), 7.65 (1H, dt, J = 6.9, 2.1 Hz), 7.80 |
| | (1H, td, J = 2.0, 0.6 Hz), 8.46 (1H, d, J = 5.3 Hz), 8.56 (1H, s), 9.11 (1H, t, J = 6.0 Hz) |
| 1155 | 2.54 (2H, d, J = 6.2 Hz), 2.68 (2H, t, J = 5.9 Hz), 2.77 (2H, d, J = 5.8 Hz), 3.18 (2H, t, J = 6.6 |
| | Hz), 3.56 (4H, t, J = 2.8 Hz), 3.76 (3H, d, J = 2.2 Hz), 4.01 (2H, t, J = 6.6 Hz), 4.40 (2H, d, J = |
| | 5.8 Hz), 4.46 (1H, t, J = 5.4 Hz), 7.03 (1H, s), 7.10 (2H, d, J = 1.3 Hz), 7.48-7.52 (2H, m), |
| | 7.62 (1H, dt, J = 6.6, 2.2 Hz), 7.78 (1H, dt, J = 2.4, 1.3 Hz), 7.87 (1H, d, J = 5.4 Hz), 8.64 (1H, |
| | t, J = 5.8 Hz) |
| 1156 | 2.54 (2H, t, J = 6.2 Hz), 2.67-2.70 (2H, m), 2.78 (2H, t, J = 5.8 Hz), 3.18-3.20 (1H, m), |
| | 3.21-3.23 (3H, m), 3.54-3.58 (4H, m), 4.02 (2H, t, J = 6.6 Hz), 4.24-4.42 (4H, m), 4.47 (2H, t, |
| | J = 5.4 Hz), 6.12 (1H, s), 7.03 (1H, d, J = 1.7 Hz), 7.10 (2H, d, J = 1.5 Hz), 7.46-7.54 (3H, m), |
| | 7.62 (1H, dt, J = 6.8, 2.1 Hz), 7.78 (1H, td, J = 1.9, 0.7 Hz), 8.83 (1H, d, J = 60.1 Hz) |
| 1157 | 1.97 (3H, d, J = 0.9 Hz), 2.53 (2H, t, J = 6.2 Hz), 2.65-2.71 (2H, m), 2.77 (2H, t, J = 5.6 Hz), |
| | 3.16-3.20 (2H, m), 3.53-3.59 (4H, m), 3.70 (3H, s), 4.01 (2H, t, J = 6.7 Hz), 4.36 (2H, d, J = |
| | 5.9 Hz), 4.46 (1H, t, J = 5.4 Hz), 7.03 (1H, t, J = 1.3 Hz), 7.09 (2H, d, J = 1.5 Hz), 7.36 (1H, d, |
| | J = 0.9 Hz), 7.47-7.52 (2H, m), 7.61 (1H, dt, J = 6.7, 2.2 Hz), 7.77 (1H, td, J = 1.8, 0.7 Hz), |
| | 8.52 (1H, t, J = 5.9 Hz) |
| 1158 | 1.07 (3H, t, J = 7.1 Hz), 1.23 (3H, d, J = 6.9 Hz), 2.30-2.35 (1H, m), 2.36 (3H, s), 2.40-2.48 |
| | (2H, m), 2.70 (1H, dd, J = 11.5, 5.1 Hz), 2.93 (1H, q, J = 6.3 Hz), 3.19 (2H, t, J = 6.6 Hz), 3.49 |
| | (2H, s), 4.03 (2H, t, J = 6.6 Hz), 4.48 (2H, d, J = 6.0 Hz), 7.03 (1H, d, J = 2.2 Hz), 7.13 (1H, |
| | dd, J = 8.2, 2.3 Hz), 7.19 (1H, d, J = 4.9 Hz), 7.23 (1H, d, J = 8.4 Hz), 7.46-7.56 (2H, m), |
| | 7.62 (1H, dt, J = 7.0, 2.0 Hz), 7.78 (1H, dt, J = 2.4, 1.2 Hz), 8.32 (1H, d, J = 4.9 Hz), 8.43 (1H, |
| | s), 8.99 (1H, t, J = 6.0 Hz) |
| 1159 | 2.36 (3H, s), 2.97 (2H, t, J = 6.6 Hz), 3.03 (3H, s), 3.22 (2H, t, J = 6.6 Hz), 3.55 (2H, t, J = 6.7 |
| | Hz), 4.06 (2H, t, J = 6.6 Hz), 4.48 (2H, d, J = 6.0 Hz), 7.19 (1H, d, J = 5.0 Hz), 7.31 (1H, d, J = |
| | 8.2 Hz), 7.45 (1H, dd, J = 8.1, 2.3 Hz), 7.47-7.54 (2H, m), 7.64 (1H, dt, J = 6.6, 2.1 Hz), |
| | 7.76-7.85 (2H, m), 8.32 (1H, d, J = 4.9 Hz), 8.44 (1H, s), 8.99 (1H, t, J = 6.0 Hz) |
| 1160 | 2.52-2.60 (2H, m), 2.69 (2H, t, J = 5.7 Hz), 2.78 (2H, d, J = 5.7 Hz), 3.21 (2H, t, J = 6.7 Hz), |
| | 3.52-3.68 (4H, m), 3.88 (3H, s), 4.03 (2H, t, J = 6.6 Hz), 4.46 (1H, t, J = 5.5 Hz), 4.57 (2H, d, |
| | J = 5.4 Hz), 7.04 (1H, s), 7.11 (2H, s), 7.31 (1H, dd, J = 8.3, 4.7 Hz), 7.44 (1H, d, J = 7.4 Hz), |
| | 7.47-7.59 (2H, m), 7.63 (1H, dt, J = 6.6, 2.1 Hz), 7.78 (1H, d, J = 2.2 Hz), 8.07-8.15 (1H, |
| | m), 8.56 (1H, t, J = 5.3 Hz) |
| 1161 | 2.52 (2H, d, J = 1.8 Hz), 2.68 (2H, s), 2.79 (2H, s), 3.19 (2H, t, J = 6.5 Hz), 3.58 (4H, s), 4.02 |
| | (2H, t, J = 6.6 Hz), 4.47 (1H, s), 4.54 (2H, d, J = 6.1 Hz), 7.05 (1H, s), 7.11 (2H, s), 7.31-7.42 |
| | (1H, m), 7.45-7.59 (2H, m), 7.64 (1H, dt, J = 6.8, 2.1 Hz), 7.80 (1H, t, J = 2.0 Hz), 8.39 (1H, |
| | dd, J = 4.8, 1.1 Hz), 8.52 (1H, d, J = 1.7 Hz), 9.15 (1H, t, J = 6.2 Hz) |
| 1162 | 2.27-2.30 (1H, m), 2.59-2.71 (3H, m), 2.74 (2H, d, J = 5.8 Hz), 3.15 (2H, t, J = 6.6 Hz), |
| | 3.54 (4H, q, J = 6.2 Hz), 3.99 (2H, t, J = 6.6 Hz), 4.42 (1H, s), 4.64 (2H, d, J = 6.0 Hz), 7.00 |
| | (1H, s), 7.07 (2H, d, J = 1.3 Hz), 7.44-7.52 (2H, m), 7.60-7.63 (1H, m), 7.70 (1H, d, J = 5.1 |
| | Hz), 7.76 (1H, d, J = 2.0 Hz), 8.72 (1H, d, J = 5.0 Hz), 8.76 (1H, s), 9.15 (1H, t, J = 6.0 Hz) |
| 1163 | 2.27 (3H, s), 2.48-2.54 (2H, m), 2.66 (2H, t, J = 5.8 Hz), 2.75 (2H t, J = 5.7 Hz), 3.15 (2H, t, |
| | J = 6.6 Hz), 3.52-3.55 (4H, m), 3.98 (2H, t, J = 6.6 Hz), 4.40 (2H, d, J = 6.1 Hz), 7.00 (1H, s), |
| | 7.07 (2H, d, J = 1.3 Hz), 7.16 (1H, d, J = 5.0 Hz), 7.45-7.52 (2H m), 7.61 (1H, dt, J = 6.6, 2.2 |
| | Hz), 7.76 (1H, t, J = 2.1 Hz), 8.31 (2H, d, J = 5.0 Hz), 9.03 (1H, t, J = 6.1 Hz). |
| 1164 | 1.07 (3H, s), 1.22 (3H, s), 1.38 (2H, s), 1.48 (4H, s), 2.06-2.24 (2H, m), 2.31 (4H, s), 3.19 |
| | (2H, q, J = 6.5 Hz), 3.41 (2H, s), 3.70 (1H, dt, J = 8.5, 7.6 Hz), 3.84 (1H, td, J = 8.5, 4.7 Hz), |
| | 4.00-4.13 (2H, m), 4.27-4.36 (1H, m), 7.17 (1H, d, J = 7.5 Hz), 7.21-7.28 (2H, m), 7.33 |
| | (1H, t, J = 7.7 Hz), 7.48-7.56 (2H, m), 7.64 (1H, dt, J = 6.9, 2.1 Hz), 7.80 (1H, td, J = 1.9, 0.6 |
| | Hz), 8.27 (1H, d, J = 8.8 Hz) |
| 1166 | 2.19 (3H, d, J = 0.8 Hz), 2.55 (2H, t, J = 6.2 Hz), 2.70 (2H, t, J = 5.7 Hz), 2.78 (2H, d, J = 5.8 |
| | Hz), 3.19 (2H, t, J = 6.6 Hz), 3.55-3.60 (4H, m), 3.65 (3H, s), 4.00-4.05 (2H, m), 4.31 (2H, |
| | d, J = 6.1 Hz), 4.48 (1H, t, J = 5.4 Hz), 5.94 (1H, d, J = 0.8 Hz), 7.04 (1H, d, J = 1.7 Hz), 7.11 |
| | (2H, d, J = 1.3 Hz), 7.49-7.54 (2H, m), 7.63 (1H, dt, J = 6.7, 2.2 Hz), 7.79 (1H, td, J = 1.8, |
| | 0.7 Hz), 8.69 (1H, t, J = 6.1 Hz) |
| 1167 | 2.55 (2H, t, J = 6.2 Hz), 2.68-2.72 (2H, m), 2.78 (2H, d, J = 5.9 Hz), 3.21 (2H, d, J = 6.5 Hz), |
| | 3.56-3.59 (4H, m), 4.03 (2H, t, J = 6.6 Hz), 4.33 (2H, d, J = 6.0 Hz), 4.48 (1H, t, J = 5.4 Hz), |
| | 7.04 (1H, s), 7.11 (2H, d, J = 1.7 Hz), 7.50-7.54 (2H, m), 7.62 (1H, dt, J = 7.0, 2.0 Hz), 7.78 |
| | (1H, dt, J = 2.4, 1.2 Hz), 8.57 (1H, s), 8.86 (1H, d, J = 0.8 Hz), 8.97 (1H, t, J = 6.1 Hz) |
| 1168 | 1.38 (2H, s), 1.48 (4H, s), 2.18 (3H, s), 2.32 (4H, s), 3.21 (2H, t, J = 6.6 Hz), 3.40 (2H, d, J = |
| | 8.3 Hz), 4.07 (2H, t, J = 6.6 Hz), 4.36 (2H, s), 5.91 (1H, s), 7.18 (1H, d, J = 7.5 Hz), 7.22- |
| | 7.30 (2H, m), 7.34 (1H, t, J = 7.7 Hz), 7.47-7.55 (2H, m), 7.63 (1H, dt, J = 6.8, 2.1 Hz), 7.80 |
| | (1H, t, J = 2.0 Hz), 8.68 (1H, s), 12.22 (1H, s) |
| 1169 | 0.21-0.27 (2H, m), 0.39-0.45 (2H, m), 1.00-1.05 (1H, m), 1.08 (3H, t, J = 7.2 Hz), 2.46 |
| | (2H, q, J = 7.2 Hz), 2.62-2.67 (2H, m), 2.71 (2H, t, J = 5.8 Hz), 3.12 (2H, t, J = 6.4 Hz), 3.19 |
| | (2H, t, J = 6.6 Hz), 3.31 (3H, s), 3.51 (2H, s), 4.02 (2H, t, J = 6.6 Hz), 4.36 (2H, s), 7.00 (1H, |
| | d, J = 2.2 Hz), 7.13 (1H, d, J = 2.2 Hz), 7.47-7.56 (2H, m), 7.62 (1H, dt, J = 6.8, 2.1 Hz), 7.77 |
| | (1H, td, J = 1.9, 0.7 Hz), 8.49 (1H, t, J = 6.0 Hz) |
| 1170 | 2.49-2.56 (2H, m), 2.63-2.73 (2H, m), 2.74-2.84 (2H, m), 3.20 (3H, t, J = 6.5 Hz), 3.58 |
| | (3H, s), 4.03 (2H, t, J = 6.6 Hz), 4.41-4.49 (1H, m), 4.68 (2H, d, J = 5.6 Hz), 7.12 (2H, s), |
| | 7.38 (1H, dd, J = 8.1, 4.7 Hz), 7.48-7.58 (2H, m), 7.63 (1H, dt, J = 6.5, 2.1 Hz), 7.79 (1H, d, |
| | J = 2.0 Hz), 7.94 (1H, dd, J = 8.1, 1.5 Hz), 8.51 (1H, dd, J = 4.7, 1.4 Hz), 8.76 (1H, d, J = 5.7 |
| | Hz). |
| 1171 | 2.44 (3H, s), 2.53-2.57 (2H, m), 2.70 (2H, t, J = 5.7 Hz), 2.79 (2H, t, J = 5.6 Hz), 3.19 (2H, t, |
| | J = 6.6 Hz), 3.53-3.61 (5H, m), 4.02 (2H, t, J = 6.6 Hz), 4.42 (2H, d, J = 6.2 Hz), 7.00-7.20 |
| | (4H, m), 7.48-7.56 (2H, m), 7.59-7.68 (1H, m), 7.77-7.81 (1H, m), 8.28 (1H, s), 8.36 (1H, |
| | dd, J = 5.1, 0.8 Hz), 9.09 (1H, t, J = 6.3 Hz) |
| 1172 | 2.53-2.57 (2H, m), 2.70 (2H, d, J = 5.5 Hz), 2.79 (2H, t, J = 5.8 Hz), 3.19 (2H, t, J = 6.6 Hz), |
| | 3.54-3.60 (5H, m), 3.95 (3H, s), 4.02 (2H, t, J = 6.6 Hz), 4.43 (2H, d, J = 6.2 Hz), 7.04 (1H, |
| | s), 7.08-7.13 (2H, m), 7.16 (1H, d, J = 4.7 Hz), 7.49-7.56 (2H, m), 7.63-7.68 (1H, m), |
| | 7.79-7.83 (1H, m), 8.17 (1H, d, J = 4.8 Hz), 8.32 (1H, s), 8.98 (1H, t, J = 6.2 Hz) |
| 1173 | 1.37 (2H, s), 1.48 (4H, t, J = 5.6 Hz), 2.31 (4H, s), 3.20 (2H, t, J = 6.6 Hz), 3.40 (2H, s), 3.82 |
| | (3H, s), 4.06 (2H, t, J = 6.6 Hz), 4.37 (2H, d, J = 6.2 Hz), 6.79 (1H, d, J = 8.5 Hz), 7.17 (1H, d, |
| | J = 7.5 Hz), 7.20-7.28 (2H, m), 7.33 (1H, t, J = 7.7 Hz), 7.45-7.56 (2H, m), 7.62 (1H, dt, J = |
| | 7.0, 2.0 Hz), 7.69 (1H, dd, J = 8.6, 2.5 Hz), 7.78 (1H, t, J = 2.0 Hz), 8.13 (1H, d, J = 2.4 Hz), |
| | 9.03 (1H, t, J = 6.3 Hz) |
| 1174 | 1.37 (2H, t, J = 7.0 Hz), 1.45-1.53 (4H, m), 2.31 (4H, s), 3.20 (2H, t, J = 6.6 Hz), 3.40 (2H, |
| | s), 4.07 (2H, t, J = 6.6 Hz), 4.57 (2H, d, J = 5.9 Hz), 7.17 (1H, dt, J = 7.7, 1.4 Hz), 7.20-7.28 |
| | (2H, m), 7.33 (1H, t, J = 7.7 Hz), 7.48-7.58 (3H, m), 7.65 (1H, dt, J = 7.0, 2.0 Hz), 7.81 (1H, |
| | dt, J = 2.4, 1.2 Hz), 8.45 (1H, d, J = 5.3 Hz), 8.55 (1H, d, J = 0.6 Hz), 9.11 (1H, t, J = 6.0 Hz) |
| 1175 | 0.20-0.29 (2H, m), 0.38-0.46 (2H, m), 1.00-1.10 (1H, m), 2.72-2.78 (3H, m), 2.77- |
| | 2.84 (3H, m), 3.12 (2H, t, J = 6.5 Hz), 3.19 (2H, t, J = 6.6 Hz), 3.62 (2H, s), 4.02 (2H, t, J = |
| | 6.6 Hz), 4.62 (2H, dt, J = 48 (H-F), 4.9 Hz), 7.05 (1H, s), 7.12 (2H, d, J = 1.3 Hz), 7.48-7.56 |
| | (2H, m), 7.60-7.64 (1H, m), 7.76-7.80 (1H, m), 8.50 (1H, t, J = 6.0 Hz) |
| 1176 | 0.24 (2H, q, J = 4.4 Hz), 0.32-0.52 (2H, m), 1.05 (1H, s), 2.73 (2H, d, J = 4.8 Hz), 2.79 (2H, |
| | d, J = 5.4 Hz), 2.88 (2H, t, J = 6.8 Hz), 3.01 (3H, s), 3.12 (2H, t, J = 6.5 Hz), 3.20 (2H, t, J = |
| | 6.6 Hz), 3.38 (2H, t, J = 6.7 Hz), 3.61 (2H, s), 4.02 (2H, t, J = 6.6 Hz), 7.05 (1H, s), 7.12 (2H, |
| | s), 7.41-7.57 (2H, m), 7.62 (1H, dt, J = 6.7, 2.1 Hz), 7.77 (1H, d, J = 2.2 Hz), 8.50 (1H, t, J = |
| | 5.9 Hz) |
| 1177 | 1.38 (2H, s), 1.48 (4H, t, J = 5.3 Hz), 2.31 (4H, d, J = 8.0 Hz), 2.36 (3H, s), 3.21 (2H, t, J = 6.6 |
| | Hz), 3.41 (2H, s), 4.06 (2H, t, J = 6.6 Hz), 4.48 (2H, d, J = 6.0 Hz), 7.18 (2H, t, J = 6.7 Hz), |
| | 7.21-7.28 (2H, m), 7.33 (1H, t, J = 7.7 Hz), 7.47-7.55 (2H, m), 7.63 (1H, dt, J = 7.0, 2.0 |
| | Hz), 7.80 (1H, dt, J = 2.4, 1.2 Hz), 8.32 (1H, d, J = 4.9 Hz), 8.44 (1H, s), 9.02 (1H, t, J = 6.0 |
| | Hz) |
| 1178 | 1.38 (2H, s), 1.48 (4H, t, J = 5.7 Hz), 1.98 (3H, d, J = 0.9 Hz), 2.31 (4H, s), 3.21 (2H, t, J = 6.6 |
| | Hz), 3.41 (2H, s), 3.72 (3H, s), 4.06 (2H, t, J = 6.6 Hz), 4.37 (2H, d, J = 5.8 Hz), 7.17 (1H, d, |
| | J = 7.5 Hz), 7.20-7.28 (2H, m), 7.33 (1H, t, J = 7.7 Hz), 7.38 (1H, s), 7.46-7.55 (2H, m), 7.63 |
| | (1H, dt, J = 6.7, 2.2 Hz), 7.80 (1H, d, J = 2.0 Hz), 8.53 (1H, t, J = 5.8 Hz) |
| 1179 | 2.96 (2H, t, J = 6.6 Hz), 3.02 (3H, s), 3.16-3.24 (2H, m), 3.48-3.56 (3H, m), 3.62 (1H, dd, |
| | J = 8.9, 4.1 Hz), 3.90 (1H, dd, J = 9.3, 4.9 Hz), 3.97 (1H, dd, J = 9.0, 6.1 Hz), 4.05 (2H, t, J = |
| | 6.6 Hz), 4.16-4.30 (2H, m), 5.29 (1H, d, J = 4.3 Hz), 7.31 (1H, d, J = 8.2 Hz), 7.44 (1H, dd, |
| | J = 8.1, 2.3 Hz), 7.48-7.53 (2H, m), 7.64 (1H, dt, J = 6.7, 2.2 Hz), 7.80 (1H, td, J = 1.8, 0.7 |
| | Hz), 7.82 (1H, d, J = 2.3 Hz), 8.47 (1H, d, J = 7.1 Hz) |
| 1180 | 1.64 (1H, m), 1.80-2.01 (1H, m), 2.97 (2H, t, J = 6.6 Hz), 3.02 (3H, s), 3.18-3.25 (2H, m), |
| | 3.38-3.49 (2H, m), 3.51-3.61 (4H, m), 3.62-3.85 (2H, m), 3.96 (1H, m), 4.06 (2H, t, J = |
| | 6.7 Hz), 4.80 (1H, t, J = 5.6 Hz), 7.31 (1H, d, J = 8.2 Hz), 7.44 (1H, dd, J = 8.1, 2.3 Hz), 7.48- |
| | 7.54 (2H, m), 7.64 (1H, m), 7.80 (1H, d, J = 2.1 Hz), 7.82 (1H, d, J = 2.3 Hz), 8.09 (1H, dd, |
| | J = 27.9, 9.3 Hz) 1 X H not seen |
| 1181 | 2.49 (3H, s), 2.79 (2H, t, J = 6.6 Hz), 2.84 (3H, s), 3.04 (2H, t, J = 6.6 Hz), 3.36 (2H, t, J = 6.6 |
| | Hz), 3.88 (2H, t, J = 6.5 Hz), 4.58 (2H, d, J = 6.1 Hz), 7.13 (1H, d, J = 8.2 Hz), 7.27 (1H, dd, |
| | J = 8.1, 2.3 Hz), 7.30-7.37 (2H, m), 7.46 (1H, dt, J = 6.7, 2.1 Hz), 7.61-7.65 (2H, m), 9.23 |
| | (1H, t, J = 6.1 Hz) |
| 1182 | 2.97 (t, J = 6.6 Hz, 2H), 3.03 (s, 3H), 3.20 (t, J = 6.6 Hz, 2H), 3.55 (t, J = 6.6 Hz, 2H), 4.08 (t, |
| | J = 6.6 Hz, 2H), 7.32 (d, J = 8.2 Hz, 1H), 7.45 (dd, J = 8.1, 2.3 Hz, 1H), 7.48 (m, 2H), 7.48-7.56 |
| | (m, 2H), 7.60 (dt, J = 6.8, 2.1 Hz, 1H), 7.75 (t, J = 2.0 Hz, 1H), 7.83 (d, J = 2.3 Hz, 1H) |
| 1183 | 2.90-3.08 (7H, m), 3.55 (2H, t, J = 6.6 Hz), 4.06 (2H, t, J = 6.6 Hz, ), 4.57 (2H, s), 5.26 (1H, |
| | s), 7.31 (1H, d, J = 8.1 Hz), 7.40-7.49 (3H, m), 7.54 (1H, dt, J = 7.3, 2.0 Hz), 7.66 (1H, t, J = |
| | 1.9 Hz), 7.83 (1H, d, J = 2.3 Hz) |
| 1184 | 2.99 (t, J = 6.7 Hz, 2H), 3.07 (t, J = 6.7 Hz, 2H), 3.15 (s, 3H), 3.44 (s, 3H), 3.55 (t, J = 6.7 Hz, |
| | 2H), 4.13 (t, J = 6.6 Hz, 2H), 4.59 (s, 2H), 7.17 (d, J = 8.1 Hz, 1H), 7.29-7.32 (m, 2H), 7.42- |
| | 7.49 (m, 2H), 7.60 (dt, J = 2.0, 1.0 Hz, 1H), 7.96 (d, J = 2.4 Hz, 1H) |
| 1185 | 2.21 (3H, s), 2.39 (3H, s), 2.94-2.98 (2H, m), 3.02 (3H, s), 3.18-3.22 (2H, m), 3.51-3.56 |
| | (2H, m), 4.05 (2H, q, J = 6.5 Hz), 4.19 (2H, d, J = 5.9 Hz), 7.28-7.32 (1H, m), 7.43 (1H, dd, |
| | J = 8.1, 2.4 Hz), 7.48-7.54 (2H, m), 7.61 (1H, dt, J = 6.8, 2.1 Hz), 7.75-7.79 (1H, m), 7.82 |
| | (1H, dd, J = 4.5, 2.3 Hz), 8.89 (1H, t, J = 5.8 Hz) |
| 1186 | 2.98 (2H, t, J = 6.6 Hz), 3.04 (3H, s), 3.23 (2H, t, J = 6.5 Hz), 3.49 (3H, s), 3.56 (2H, t, J = 6.6 |
| | Hz), 3.82 (3H, s), 4.08 (2H, t, J = 6.6 Hz), 4.29 (2H, d, J = 6.1 Hz), 5.58 (1H, s), 7.33 (1H, d, |
| | J = 8.1 Hz), 7.46 (1H, dd, J = 8.1, 2.4 Hz), 7.50-7.55 (2H, m), 7.64-7.68 (1H, m), 7.81-7.85 |
| | (2H, m), 8.74 (1H, t, J = 6.1 Hz) |
| 1187 | 2.96 (2H, t, J = 6.6 Hz), 3.01 (3H, s), 3.15-3.19 (2H, m), 3.51-3.56 (2H, m), 3.70-3.81 |
| | (2H, m), 4.03 (2H, t, J = 6.6 Hz), 5.04-5.17 (2H, m), 7.30 (1H, d, J = 8.2 Hz), 7.35 (1H, ddd, |
| | J = 7.8, 4.8, 0.8 Hz), 7.43 (1H, dd, J = 8.0, 2.4 Hz), 7.50-7.55 (2H, m), 7.65 (1H, dt, J = 6.8, |
| | 2.1 Hz), 7.79-7.83 (3H, m), 8.45 (1H, dd, J = 4.8, 1.6 Hz), 8.60 (1H, q, J = 0.8 Hz), 8.74 (1H, |
| | d, J = 8.2 Hz) |
| 1188 | 2.15 (3H, s), 2.36 (3H, d, J = 0.7 Hz), 3.12 (2H, t, J = 8.5 Hz), 3.20 (2H, t, J = 6.6 Hz), 4.00 |
| | (2H, t, J = 6.6 Hz), 4.12 (2H, t, J = 8.5 Hz), 4.47 (2H, d, J = 5.9 Hz), 6.95 (1H, dd, J = 7.9, 2.0 |
| | Hz), 7.19 (1H, d, J = 4.9 Hz), 7.23 (1H, d, J = 8.0 Hz), 7.46-7.55 (2H, m), 7.59-7.66 (1H, |
| | m), 7.78-7.80 (1H, m), 7.99 (1H, d, J = 2.1 Hz), 8.32 (1H, d, J = 4.9 Hz), 8.43 (1H, s), 8.99 |
| | (1H, t, J = 6.0 Hz) |
| 1189 | 2.31 (3H, s), 2.38 (3H, s), 2.97 (2H, t, J = 6.6 Hz), 3.02 (3H, s), 3.21 (2H, t, J = 6.6 Hz), 3.50- |
| | 3.59 (2H, m), 4.06 (2H, t, J = 6.6 Hz), 4.39-4.46 (2H, m), 7.02-7.07 (1H, m), 7.32 (1H, d, |
| | J = 8.2 Hz), 7.45 (1H, dd, J = 8.1, 2.3 Hz), 7.47-7.54 (2H, m), 7.60-7.68 (1H, m), 7.78-7.81 |
| | (1H, m), 7.82 (1H, d, J = 2.3 Hz), 8.30 (1H, s), 8.94 (1H, t, J = 6.0 Hz). |
| 1190 | 0.21-0.26 (2H, m), 0.39-0.44 (2H, m), 0.99-1.10 (1H, m), 2.96 (2H, t, J = 6.6 Hz), 3.02 |
| | (3H, s), 3.10-3.14 (2H, m), 3.21 (2H, t, J = 6.5 Hz), 3.54 (2H, t, J = 6.6 Hz), 4.11 (2H, t, J = 6.6 |
| | Hz), 7.30-7.35 (2H, m), 7.49-7.56 (2H, m), 7.61-7.66 (1H, m), 7.77-7.81 (1H, m), |
| | 7.86 (1H, d, J = 8.2 Hz), 8.54 (1H, t, J = 6.0 Hz) |
| 1191 | 0.19-0.28 (2H, m), 0.36-0.46 (2H, m), 0.97-1.12 (1H, m), 2.53-2.64 (2H, m), 2.61- |
| | 2.72 (1H, m), 2.92-3.01 (1H, m), 3.12 (2H, t, J = 6.5 Hz), 3.20 (2H, t, J = 6.6 Hz), 3.39 (3H, |
| | s), 3.54-3.64 (4H, m), 4.04 (2H, t, J = 6.6 Hz), 4.32-4.40 (1H, m), 4.48 (1H, t, J = 5.4 Hz), |
| | 7.09 (1H, d, J = 2.2 Hz), 7.19 (1H, dd, J = 8.3, 2.2 Hz), 7.37 (1H, d, J = 8.4 Hz), 7.46-7.57 |
| | (2H, m), 7.57-7.67 (1H, m), 7.75-7.81 (1H, m), 8.51 (1H, t, J = 6.0 Hz). |
| 1192 | 0.17-0.27 (2H, m), 0.35-0.44 (2H, m), 0.96-1.06 (1H, m), 3.04 (3H, s), 3.08 (2H, t, |
| | J = 6.4 Hz), 3.20 (2H, t, J = 6.6 Hz), 4.06 (2H, t, J = 6.6 Hz), 4.43 (2H, s), 7.45-7.51 (2H, m), |
| | 7.52-7.58 (2H, m), 7.60-7.65 (2H, m), 7.76-7.79 (1H, m), 8.50 (1H, t, J = 6.0 Hz) |
| 1193 | 2.59 (3H, s), 2.98 (2H, t, J = 6.7 Hz), 3.04 (3H, s), 3.23 (2H, t, J = 6.6 Hz), 3.55 (2H, t, J = 6.7 |
| | Hz), 4.07 (2H, t, J = 6.6 Hz), 4.56 (2H, d, J = 6.1 Hz), 7.33 (1H, d, J = 8.1 Hz), 7.44-7.51 (3H, |
| | m), 7.52-7.53 (1H, m), 7.64 (1H, dt, J = 6.8, 2.1 Hz), 7.80 (1H, td, J = 1.9, 0.7 Hz), 7.84 (1H, |
| | d, J = 2.3 Hz), 9.12 (1H, t, J = 6.1 Hz) |
| 1194 | 2.38 (3H, s), 2.98 (2H, t, J = 6.7 Hz), 3.04 (3H, s), 3.23 (2H, t, J = 6.6 Hz), 3.55 (2H, q, J = 7.0 |
| | Hz), 4.07 (2H, t, J = 6.6 Hz), 4.46 (2H, d, J = 5.9 Hz), 6.86 (1H, d, J = 1.0 Hz), 7.33 (1H, d, J = |
| | 8.1 Hz), 7.46 (1H, dd, J = 8.1, 2.3 Hz), 7.50-7.55 (2H, m), 7.65 (1H, dt, J = 6.6, 2.2 Hz), 7.80 |
| | -7.85 (2H, m), 8.97 (1H, t, J = 5.9 Hz) |
| 1195 | 0.19-0.26 (2H, m), 0.34-0.48 (2H, m), 0.98-1.10 (1H, m), 2.92 (3H, s), 2.97 (3H, s), 3.12 |
| | (2H, t, J = 6.5 Hz), 3.22 (2H, t, J = 6.6 Hz), 4.09 (2H, t, J = 6.6 Hz), 7.26-7.31 (1H, m), 7.40- |
| | 7.50 (3H, m), 7.50-7.56 (2H, m), 7.64 (1H, dt, J = 6.9, 2.1 Hz), 7.79-7.82 (1H, m), 8.56 |
| | (1H, t, J = 6.0 Hz) |
| 1196 | 1.33 (3H, t, J = 7.1 Hz), 2.92 (3H, s), 2.97 (3H, s), 3.22 (2H, t, J = 6.6 Hz), 4.12 (2H, t, J = 6.6 |
| | Hz), 4.36 (2H, q, J = 7.1 Hz), 7.25-7.30 (1H, m), 7.40-7.49 (3H, m), 7.50-7.55 (1H, m), |
| | 7.56 (1H, dt, J = 8.0, 1.7 Hz), 7.61 (1H, dt, J = 7.4, 1.8 Hz), 7.76-7.79 (1H, m) |
| 1197 | 1.07 (3H, s), 1.22 (3H, s), 2.05-2.15 (1H, m), 2.17-2.27 (1H, m), 2.97 (2H, t, J = 6.6 Hz), |
| | 3.03 (3H, s), 3.17-3.25 (2H, m), 3.54 (2H, t, J = 6.6 Hz), 3.66-3.74 (1H, m), 3.80-3.87 |
| | (1H, m), 4.01-4.12 (2H, m), 4.27-4.35 (1H, m), 7.32 (1H, d, J = 8.2 Hz), 7.45 (1H, dd, J = |
| | 8.1, 2.3 Hz), 7.48-7.56 (2H, m), 7.63-7.68 (1H, m), 7.80-7.82 (1H, m), 7.83 (1H, d, J = |
| | 2.3 Hz), 8.27 (1H, d, J = 8.8 Hz) |
| 1198 | 1.56-1.66 (1H, m), 1.85-1.97 (1H, m), 2.97 (2H, t, J = 6.7 Hz), 3.03 (3H, s), 3.19-3.26 |
| | (4H, m), 3.44-3.50 (1H, m), 3.55 (2H, t, J = 6.7 Hz), 3.57-3.69 (2H, m), 3.70-3.78 (1H, |
| | m), 4.06 (2H, t, J = 6.6 Hz), 7.32 (1H, d, J = 8.1 Hz), 7.45 (1H, dd, J = 8.1, 2.3 Hz), 7.48-7.55 |
| | (2H, m), 7.62-7.66 (1H, m), 7.79-7.80 (1H, m), 7.83 (1H, d, J = 2.3 Hz), 8.62 (1H, t, J = 6.1 |
| | Hz) |
| 1199 | 2.19 (3H, d, J = 0.7 Hz), 2.97 (2H, t, J = 6.6 Hz), 3.03 (3H, s), 3.22 (2H, t, J = 6.6 Hz), 3.50- |
| | 3.59 (2H, m), 3.64 (3H, s), 4.06 (2H, t, J = 6.6 Hz), 4.31 (2H, d, J = 6.1 Hz), 5.94 (1H, d, J = |
| | 0.8 Hz), 7.32 (1H, d, J = 8.1 Hz), 7.45 (1H, dd, J = 8.1, 2.3 Hz), 7.48-7.55 (2H, m), 7.62- |
| | 7.68 (1H, m), 7.79-7.82 (1H, m), 7.83 (1H, d, J = 2.3 Hz), 8.70 (1H, t, J = 6.1 Hz) |
| 1200 | 2.16 (3H, s), 2.97 (2H, t, J = 6.6 Hz), 3.03 (3H, s), 3.22 (2H, t, J = 6.6 Hz), 3.55 (2H, t, J = 6.7 |
| | Hz), 4.07 (2H, t, J = 6.6 Hz), 4.36 (2H, d, J = 5.9 Hz), 5.91 (1H, s), 7.32 (1H, d, J = 8.2 Hz), |
| | 7.45 (1H, dd, J = 8.1, 2.3 Hz), 7.48-7.54 (2H, m), 7.62-7.67 (1H, m), 7.80-7.82 (1H, m), |
| | 7.83 (1H, d, J = 2.3 Hz), 8.59-8.83 (1H, m), 12.22 (1H, s) |
| 1201 | 2.26-2.31 (3H, m), 2.97 (2H, t, J = 6.6 Hz), 3.02 (3H, s), 3.22 (2H, t, J = 6.6 Hz), 3.54 (2H, t, |
| | J = 6.7 Hz), 4.06 (2H, t, J = 6.6 Hz), 4.43 (2H, d, J = 6.2 Hz), 7.32 (1H, d, J = 7.9 Hz), 7.45 |
| | (1H, dd, J = 8.1, 2.4 Hz), 7.48-7.55 (3H, m), 7.63-7.67 (1H, m), 7.79-7.82 (1H, m), 7.83 |
| | (1H, d, J = 2.4 Hz), 8.27-8.32 (1H, m), 8.34-8.38 (1H, m), 9.10 (1H, t, J = 6.3 Hz) |
| 1202 | 2.97 (2H, t, J = 6.6 Hz), 3.03 (3H, s), 3.22 (2H, t, J = 6.6 Hz), 3.55 (2H, t, J = 6.7 Hz), 4.07 |
| | (2H, t, J = 6.6 Hz), 4.76 (2H, d, J = 6.1 Hz), 7.32 (1H, d, J = 7.9 Hz), 7.45 (1H, dd, J = 8.1, 2.4 |
| | Hz), 7.49-7.56 (2H, m), 7.61-7.71 (3H, m), 7.81-7.85 (2H, m), 9.14 (1H, dd, J = 4.7, 1.8 |
| | Hz), 9.22 (1H, t, J = 6.1 Hz) |
| 1203 | 1.60-1.74 (4H, m), 2.97 (2H, t, J = 6.6 Hz), 3.03 (3H, s), 3.21 (2H, t, J = 6.6 Hz), 3.35-3.41 |
| | (2H, m), 3.54 (2H, t, J = 6.7 Hz), 3.83-3.92 (2H, m), 4.06 (3H, t, J = 6.6 Hz), 7.32 (1H, d, J = |
| | 8.1 Hz), 7.45 (1H, dd, J = 8.0, 2.3 Hz), 7.48-7.55 (2H, m), 7.62-7.66 (1H, m), 7.78-7.81 |
| | (1H, m), 7.83 (1H, d, J = 2.3 Hz), 8.35 (1H, d, J = 8.4 Hz) |
| 1204 | 2.52-2.57 (2H, m), 2.66-2.72 (2H, m), 2.75-2.82 (2H, m), 3.19 (2H, t, J = 6.6 Hz), 3.53- |
| | 3.61 (4H, m), 4.02 (2H, t, J = 6.6 Hz), 4.44-4.50 (1H, m), 4.76 (2H, d, J = 5.8 Hz), 7.04 (1H, |
| | s), 7.08-7.14 (2H, m), 7.47-7.57 (2H, m), 7.61-7.70 (3H, m), 7.78-7.83 (1H, m), 9.14 |
| | (1H, m), 9.21 (1H, t, J = 6.2 Hz) |
| 1205 | 2.19 (3H, d, J = 0.7 Hz), 2.96 (2H, t, J = 6.7 Hz), 3.02 (3H, s), 3.21 (2H, t, J = 6.6 Hz), 3.54 (2H, t, |
| | J = 6.7 Hz), 3.64 (3H, s), 4.11 (2H, t, J = 6.5 Hz), 4.29-4.33 (2H, m), 5.93 (1H, d, J = 0.9 Hz), |
| | 7.30-7.35 (2H, m), 7.48-7.55 (2H, m), 7.63 (1H, dt, J = 6.8, 2.1 Hz), 7.80 (1H, dt, J = 2.3, |
| | 1.2 Hz), 7.86 (1H, d, J = 8.3 Hz), 8.71 (1H, t, J = 6.1 Hz). |
| 1206 | 2.19 (3H, s), 3.08 (3H, s), 3.24 (2H, t, J = 6.3 Hz), 3.65 (4H, s), 4.10 (3H, t, J = 6.6 Hz), 4.31 |
| | (2H, d, J = 5.9 Hz), 4.47 (3H, s), 5.94 (1H, s), 7.47-7.55 (2H, m), 7.55-7.63 (2H, m), 7.63- |
| | 7.70 (2H, m), 7.79-7.84 (1H, m), 8.71 (1H, t, J = 6.0 Hz) |
| 1207 | 0.14-0.28 (2H, m), 0.31-0.48 (2H, m), 0.94-1.09 (1H, m), 2.48-2.53 (2H, m), 2.79- |
| | 2.85 (2H, m), 3.05-3.11 (2H, m), 3.15-3.20 (5H, m), 4.03 (2H, t, J = 6.6 Hz), 6.94-6.98 |
| | (1H, m), 7.05 (1H, d, J = 2.0 Hz), 7.17-7.22 (1H, m), 7.42-7.55 (2H, m), 7.57-7.62 (1H, |
| | m), 7.73-7.77 (1H, m), 8.50 (1H, t, J = 6.0 Hz) |
| 1208 | 2.17-2.20 (3H, m), 2.52-2.56 (2H, m), 2.82-2.90 (2H, m), 3.18-3.24 (5H, m), 3.64 |
| | (3H, s), 4.07 (2H, t, J = 6.6 Hz), 4.29-4.33 (2H, m), 5.92-5.95 (1H, m), 6.97-7.02 (1H, m), |
| | 7.09 (1H, d, J = 2.0 Hz), 7.22-7.27 (1H, m), 7.47-7.55 (2H, m), 7.61-7.66 (1H, m), 7.78- |
| | 7.82 (1H, m), 8.71 (1H, t, J = 6.1 Hz) |
| 1209 | 0.86 (3H, t, J = 7.4 Hz), 1.51-1.61 (2H, m), 2.36 (3H, d, J = 0.6 Hz), 2.95 (2H, t, J = 6.6 Hz), |
| | 3.22 (2H, t, J = 6.6 Hz), 3.41-3.47 (2H, m), 3.50-3.56 (2H, m), 4.06 (2H, t, J = 6.6 Hz), 4.48 |
| | (2H, d, J = 6.0 Hz), 7.16-7.22 (1H, m), 7.32 (1H, d, J = 8.2 Hz), 7.45 (1H, dd, J = 8.1, 2.3 Hz), |
| | 7.47-7.54 (2H, m), 7.59-7.68 (1H, m), 7.79-7.81 (1H, m), 7.82 (1H, d, J = 2.4 Hz), 8.32 |
| | (1H, d, J = 5.0 Hz), 8.43 (1H, s), 9.01 (1H, t, J = 6.0 Hz). |
| 1210 | 0.87 (3H, t, J = 7.4 Hz), 1.51-1.61 (2H, m), 2.19 (3H, d, J = 0.7 Hz), 2.96 (2H, t, J = 6.5 Hz), |
| | 3.22 (2H, t, J = 6.6 Hz), 3.40-3.47 (2H, m), 3.50-3.57 (2H, m), 3.64 (3H, s), 4.06 (2H, t, |
| | J = 6.6 Hz), 4.31 (2H, d, J = 6.1 Hz), 5.94 (1H, d, J = 0.8 Hz), 7.32 (1H, d, J = 8.1 Hz), 7.45 (1H, dd, |
| | J = 8.1, 2.3 Hz), 7.47-7.54 (2H, m), 7.61-7.68 (1H, m), 7.79-7.82 (1H, m), 7.82-7.85 |
| | (1H, m), 8.70 (1H, t, J = 6.1 Hz) |
| 1211 | 0.21-0.26 (2H, m), 0.39-0.45 (2H, m), 0.87 (3H, t, J = 7.4 Hz), 0.99-1.10 (1H, m), 1.51- |
| | 1.62 (2H, m), 2.96 (2H, t, J = 6.5 Hz), 3.09-3.15 (2H, m), 3.22 (2H, t, J = 6.6 Hz), 3.41-3.47 |
| | (2H, m), 3.51-3.57 (2H, m), 4.06 (2H, t, J = 6.6 Hz), 7.32 (1H, d, J = 8.2 Hz), 7.45 (1H, dd, |
| | J = 8.1, 2.3 Hz), 7.48-7.55 (2H, m), 7.61-7.68 (1H, m), 7.79-7.82 (1H, m), 7.82-7.84 |
| | (1H, m), 8.52 (1H, t, J = 6.0 Hz). |
| 1212 | 2.36 (3H, s), 3.19-3.28 (2H, m), 3.51 (3H, s), 4.15 (2H, t, J = 6.6 Hz), 4.48 (2H, d, J = 6.0 |
| | Hz), 6.63 (1H, d, J = 7.2 Hz), 7.19 (1H, d, J = 4.9 Hz), 7.43-7.56 (3H, m), 7.62-7.74 (3H, |
| | m), 7.80-7.84 (1H, m), 8.16 (1H, d, J = 2.1 Hz), 8.32 (1H, d, J = 4.9 Hz), 8.44 (1H, s), 9.02 |
| | (1H, t, J = 6.0 Hz) |
| 1213 | 2.96 (2H, t, J = 6.6 Hz), 3.02 (3H, s), 3.21 (2H, t, J = 6.6 Hz), 3.54 (2H, t, J = 6.6 Hz), 3.80 |
| | (3H, s), 4.05 (2H, t, J = 6.6 Hz), 4.45 (2H, d, J = 6.2 Hz), 7.28-7.34 (2H, m), 7.44 (1H, dd, J = |
| | 8.1, 2.3 Hz), 7.48-7.54 (2H, m), 7.64 (1H, dt, J = 6.8, 2.1 Hz), 7.81 (2H, dd, J = 6.0, 2.2 Hz), |
| | 8.15 (1H, d, J = 1.7 Hz), 8.18 (1H, d, J = 2.8 Hz), 9.12 (1H, t, J = 6.3 Hz) |
| 1214 | 1.53 (3H, d, J = 7.1 Hz), 2.96 (2H, t, J = 6.6 Hz), 3.02 (3H, s), 3.14-3.21 (2H, m), 3.54 (2H, t, |
| | J = 6.6 Hz), 4.00-4.07 (2H, m), 5.22 (1H, p, J = 7.2 Hz), 7.31 (1H, d, J = 8.2 Hz), 7.36 (1H, |
| | m), 7.44 (1H, dd, J = 8.1, 2.4 Hz), 7.49-7.55 (2H, m), 7.65 (1H, dt, J = 6.8, 2.1 Hz), 7.79- |
| | 7.85 (3H, m), 8.44 (1H, dd, J = 4.8, 1.7 Hz), 8.62 (1H, d, J = 2.3 Hz), 8.95 (1H, d, J = 8.3 Hz) |
| 1215 | 1.54 (3H, d, J = 7.2 Hz), 2.97 (2H, t, J = 6.6 Hz), 3.03 (3H, s), 3.19 (2H, dt, J = 8.1, 3.4 Hz), |
| | 3.55 (2H, t, J = 6.7 Hz), 4.01-4.07 (2H, m), 5.22 (1H, p, J = 7.2 Hz), 7.32 (1H, d, J = 8.2 Hz), |
| | 7.37 (1H, m), 7.44 (1H, dd, J = 8.0, 2.4 Hz), 7.48-7.57 (2H, m), 7.66 (1H, dt, J = 6.8, 2.0 |
| | Hz), 7.81-7.83 (2H, m), 7.85 (1H, t, J = 2.1 Hz), 8.45 (1H, dd, J = 4.8, 1.6 Hz), 8.63 (1H, d, |
| | J = 2.2 Hz), 8.96 (1H, d, J = 8.3 Hz) |
| 1216 | 0.22-0.26 (2H, m), 0.39-0.44 (2H, m), 0.99-1.10 (1H, m), 2.95 (2H, t, J = 6.5 Hz), 3.08- |
| | 3.16 (2H, m), 3.22 (2H, t, J = 6.6 Hz), 3.52-3.64 (6H, m), 4.06 (2H, t, J = 6.6 Hz), 4.78 |
| | (1H, s), 7.32 (1H, d, J = 8.2 Hz), 7.45 (1H, dd, J = 8.1, 2.3 Hz), 7.48-7.55 (2H, m), 7.61- |
| | 7.68 (1H, m), 7.79-7.82 (1H, m), 7.82-7.84 (1H, m), 8.52 (1H, t, J = 6.0 Hz). |
| 1217 | 1.98 (3H, s), 2.88-3.02 (2H, m), 3.17-3.26 (2H, m), 3.51-3.64 (6H, m), 3.72 (3H, m), |
| | 4.00-4.15 (2H, m), 4.30-4.43 (2H, m), 4.73-4.81 (1H, m), 7.30-7.34 (1H, m), 7.38 |
| | (1H, s), 7.41-7.56 (3H, m), 7.61-7.69 (1H, m), 7.78-7.85 (2H, m), 8.49-8.58 (1H, m). |
| 1218 | 2.54 (2H, t, J = 6.3 Hz), 2.66-2.72 (2H, m), 2.76-2.82 (2H, m), 3.18 (2H, t, J = 6.6 Hz), |
| | 3.54-3.60 (4H, m), 4.02 (2H, t, J = 6.6 Hz), 4.45-4.53 (3H, m), 7.02-7.04 (1H, m), 7.09-7.12 |
| | (2H, m), 7.49-7.55 (2H, m), 7.56-7.59 (1H, m), 7.62-7.68 (1H, m), 7.77-7.82 (1H, m), |
| | 9.16 (1H, dd, J = 5.3, 1.3 Hz), 9.18-9.25 (2H, m) |
| 1219 | 1.10 (3H, t, J = 7.1 Hz), 2.18-2.21 (3H, m), 2.96 (2H, t, J = 6.5 Hz), 3.22 (2H, t, J = 6.6 Hz), |
| | 3.47-3.57 (4H, m), 3.64 (3H, s), 4.06 (2H, t, J = 6.6 Hz), 4.31 (2H, d, J = 6.1 Hz), 5.80-6.02 |
| | (1H, m), 7.29-7.35 (1H, m), 7.42-7.48 (1H, m), 7.49-7.54 (2H, m), 7.61-7.68 (1H, m), |
| | 7.79-7.81 (1H, m), 7.83 (1H, d, J = 2.3 Hz), 8.70 (1H, t, J = 6.1 Hz). |
| 1220 | 2.25 (3H, d, J = 0.7 Hz), 2.71 (2H, t, J = 5.9 Hz), 2.86 (2H, t, J = 5.5 Hz), 2.92 (2H, d, J = 5.5 |
| | Hz), 3.12 (1H, m), 3.25 (2H, dd, J = 3.4, 1.8 Hz), 3.47 (1H, m), 3.72 (3H, s), 3.75 (4H, dd, J = |
| | 10.3, 4.5 Hz), 4.29 (2H, t, J = 6.6 Hz), 4.47 (2H, s), 6.03 (1H, d, J = 0.8 Hz), 7.36 (1H, s), |
| | 7.44-7.48 (2H, m), 7.58 (1H, m), 7.72 (1H, dt, J = 2.2, 0.9 Hz), 8.23 (1H, d, J = 0.8 Hz) |
| 1221 | 2.52-2.59 (2H, m), 2.70-2.75 (2H, m), 2.77-2.83 (2H, m), 3.17 (2H, t, J = 6.5 Hz), 3.53- |
| | 3.60 (4H, m), 3.88 (3H, s), 4.29 (2H, t, J = 6.5 Hz), 4.46-4.52 (1H, m), 4.57 (2H, d, J = 5.4 |
| | Hz), 7.31 (1H, dd, J = 8.3, 4.8 Hz), 7.37-7.39 (1H, m), 7.43 (1H, dd, J = 8.3, 1.3 Hz), 7.52- |
| | 7.60 (2H, m), 7.63-7.68 (1H, m), 7.81-7.84 (1H, m), 8.10 (1H, dd, J = 4.8, 1.3 Hz), 8.21- |
| | 8.23 (1H, m), 8.57 (1H, t, J = 5.4 Hz) |
| 1222 | 2.19 (3H, d, J = 0.7 Hz), 2.95 (2H, t, J = 6.5 Hz), 3.22 (2H, t, J = 6.6 Hz), 3.52-3.59 (4H, m), |
| | 3.59-3.64 (2H, m), 3.64 (3H, s), 4.06 (2H, t, J = 6.6 Hz), 4.31 (2H, d, J = 6.0 Hz), 4.75-4.80 |
| | (1H, m), 5.94 (1H, d, J = 0.8 Hz), 7.30-7.34 (1H, m), 7.45 (1H, dd, J = 8.1, 2.3 Hz), 7.48- |
| | 7.53 (2H, m), 7.63-7.67 (1H, m), 7.80-7.82 (1H, m), 7.83 (1H, d, J = 2.3 Hz), 8.69 (1H, t, |
| | J = 6.1 Hz) |
| 1223 | 2.55 (2H, t, J = 6.2 Hz), 2.69 (2H, dd, J = 8.6, 3.6 Hz), 2.78 (2H, d, J = 5.9 Hz), 3.19 (2H, t, J = |
| | 6.7 Hz), 3.53-3.61 (4H, m), 4.02 (2H, t, J = 6.7 Hz), 4.36 (2H, dd, J = 6.0, 1.2 Hz), 4.48 (1H, |
| | t, J = 5.4 Hz), 7.04 (1H, t, J = 1.3 Hz), 7.11 (2H, d, J = 1.3 Hz), 7.47-7.55 (2H, m), 7.63 (1H, |
| | dt, J = 6.7, 2.1 Hz), 7.79 (1H, td, J = 1.9, 0.7 Hz), 7.94 (1H, q, J = 1.1 Hz), 8.32 (1H, d, J = 1.0 |
| | Hz), 8.85 (1H, t, J = 6.0 Hz) |
| 1224 | 1.10 (3H, t, J = 7.1 Hz), 1.98 (3H, d, J = 0.8 Hz), 2.96 (2H, t, J = 6.6 Hz), 3.22 (2H, t, J = 6.6 |
| | Hz), 3.46-3.59 (4H, m), 3.72 (3H, s), 4.06 (2H, t, J = 6.6 Hz), 4.37 (2H, d, J = 5.9 Hz), 7.32 |
| | (1H, d, J = 8.2 Hz), 7.38 (1H, d, J = 0.9 Hz), 7.43-7.48 (1H, m), 7.48-7.53 (2H, m), 7.61- |
| | 7.68 (1H, m), 7.77-7.84 (1H, m), 7.83 (1H, d, J = 2.4 Hz), 8.54 (1H, t, J = 5.9 Hz) |
| 1225 | 1.98 (3H, d, J = 0.8 Hz), 2.97 (2H, t, J = 6.6 Hz), 3.03 (3H, s), 3.22 (2H, t, J = 6.6 Hz), 3.55 |
| | (2H, t, J = 6.7 Hz), 3.72 (3H, s), 4.06 (2H, t, J = 6.6 Hz), 4.37 (2H, d, J = 5.9 Hz), 7.32 (1H, d, |
| | J = 8.1 Hz), 7.38 (1H, d, J = 0.9 Hz), 7.43-7.48 (1H, m), 7.49-7.53 (2H, m), 7.61-7.67 (1H, |
| | m), 7.80-7.82 (1H, m), 7.83 (1H, d, J = 2.4 Hz), 8.53 (1H, t, J = 5.8 Hz) |
| 1226 | 0.18-0.30 (m, 2H), 0.35-0.52 (m, 2H), 0.97-1.13 (m, 1H), 2.49-2.57 (m, 2H), 2.57- |
| | 2.64 (m, 2H), 2.64-2.74 (m, 3H), 3.12 (t, J = 6.4 Hz, 2H), 3.19 (t, J = 6.6 Hz, 2H), 3.48- |
| | 3.62 (m, 4H), 3.74 (s, 3H), 4.03 (t, J = 6.6 Hz, 2H), 4.47 (t, J = 5.4 Hz, 1H), 6.64 (d, J = 1.9 |
| | Hz, 1H), 6.76 (d, J = 1.9 Hz, 1H), 7.43-7.57 (m, 2H), 7.59-7.68 (m, 1H), 7.77 (d, J = 1.8 |
| | Hz, 1H), 8.52 (t, J = 6.0 Hz, 1H) |
| 1227 | 2.19 (s, 3H), 2.57 (t, J = 6.1 Hz, 2H), 2.86 (t, J = 5.9 Hz, 2H), 3.07 (t, J = 5.9 Hz, 2H), 3.22 (t, |
| | J = 6.5 Hz, 2H), 3.56 (q, J = 5.9 Hz, 2H), 3.64 (s, 3H), 3.66 (s, 2H), 4.31 (d, J = 5.8 Hz, 2H), |
| | 4.42 (t, J = 6.5 Hz, 2H), 4.51 (t, J = 5.4 Hz, 1H), 5.94 (s, 1H), 7.46-7.62 (m, 2H), 7.61-7.71 |
| | (m, 2H), 7.84 (t, J = 2.0 Hz, 1H), 8.72 (t, J = 6.1 Hz, 1H). |
| 1229 | 2.58 (t, J = 6.1 Hz, 2H), 2.86 (t, J = 6.0 Hz, 2H), 3.07 (t, J = 5.8 Hz, 2H), 3.23 (t, J = 6.5 Hz, |
| | 2H), 3.56 (q, J = 5.9 Hz, 2H), 3.66 (s, 2H), 3.88 (s, 3H), 4.43 (t, J = 6.5 Hz, 2H), 4.52 (t, J = |
| | 5.4 Hz, 1H), 4.57 (d, J = 5.5 Hz, 2H), 7.28-7.35 (m, 1H), 7.44 (dd, J = 8.4, 1.3 Hz, 1H), |
| | 7.52-7.59 (m, 2H), 7.65 (d, J = 0.9 Hz, 1H), 7.65-7.69 (m, 1H), 7.85 (td, J = 1.9, 0.7 Hz, 1H), |
| | 8.11 (dd, J = 4.8, 1.3 Hz, 1H), 8.60 (t, J = 5.4 Hz, 1H). |
| 1230 | 2.53-2.59 (m, 2H), 2.67-2.73 (m, 2H), 2.78 (d, J = 5.7 Hz, 2H), 3.20 (t, J = 6.6 Hz, 2H), |
| | 3.54-3.61 (m, 4H), 3.80 (s, 3H), 4.03 (t, J = 6.6 Hz, 2H), 4.51 (d, J = 5.8 Hz, 2H), 6.88 (d, J = |
| | 5.4 Hz, 2H), 7.04 (s, 1H), 7.11 (d, J = 1.4 Hz, 2H), 7.46-7.60 (m, 2H), 7.64 (dt, J = 6.6, 2.2 |
| | Hz, 1H), 7.80 (dt, J = 2.4, 1.3 Hz, 1H), 8.33 (dd, J = 5.3, 1.0 Hz, 1H), 8.96 (t, J = 6.0 Hz, 1H). |
| 1231 | 0.58-0.64 (2H, m), 0.82-0.88 (2H, m), 1.84 (1H, tt, J = 8.4, 5.1 Hz), 2.98 (2H, t, J = 6.6 |
| | Hz), 3.03 (3H, s), 3.22 (2H, t, J = 6.6 Hz), 3.54 (2H, t, J = 7.0 Hz), 4.07 (2H, t, J = 6.6 Hz), |
| | 4.36 (2H, d, J = 6.0 Hz), 5.82 (1H, s), 7.32 (1H, d, J = 8.2 Hz), 7.45 (1H, dd, J = 8.1, 2.4 Hz), |
| | 7.47-7.55 (2H, m), 7.60-7.71 (1H, m), 7.78-7.86 (2H, m), 8.71 (1H, s br), 12.21 (1H, s |
| | br) |
| 1232 | 1.41 (2H, d, J = 7.7 Hz), 1.54 (4H, q, J = 5.7 Hz), 3.14 (2H, t, J = 6.6 Hz), 3.59-3.63 (2H, m), |
| | 4.00 (2H, t, J = 6.6 Hz), 7.20 (1H, d, J = 7.5 Hz), 7.25-7.30 (1H, m), 7.33-7.39 (2H, m), |
| | 7.44-7.55 (3H, m), 7.66 (1H, d, J = 2.1 Hz), 8.22 (OH, s) |
| 1233 | 2.98 (2H, t, J = 6.6 Hz), 3.03 (3H, s), 3.23 (2H, t, J = 6.6 Hz), 3.55 (2H, t, J = 6.7 Hz), 3.88 |
| | (3H, s), 4.07 (2H, t, J = 6.6 Hz), 4.57 (2H, d, J = 5.3 Hz), 7.27-7.35 (2H, m), 7.40-7.48 (2H, |
| | m), 7.49-7.56 (2H, m), 7.61-7.68 (1H, m), 7.79-7.85 (2H, m), 8.11 (1H, dd, J = 4.8, 1.3 |
| | Hz), 8.58 (1H, t, J = 5.4 Hz) |
| 1234 | 0.55-0.66 (2H, m), 0.78-0.90 (2H, m), 1.08 (3H, t, J = 7.1 Hz), 1.83 (1H, td, J = 8.6, 4.4 |
| | Hz), 2.46-2.48 (2H, m), 2.62 (2H, t, J = 5.9 Hz), 2.80 (2H, t, J = 5.7 Hz), 3.21 (2H, t, J = 6.6 |
| | Hz), 3.44-3.55 (2H, m), 3.97 (2H, t, J = 6.5 Hz), 4.35 (2H, d, J = 6.0 Hz), 5.82 (1H, s), 7.04 |
| | (1H, d, J = 11.0 Hz), 7.16 (1H, d, J = 7.7 Hz), 7.48-7.56 (2H, m), 7.61 (1H, dt, J = 6.9, 2.1 |
| | Hz), 7.76-7.78 (1H, m), 8.73 (1H, br.s), 12.24 (1H, br. s) |
| 1235 | 2.97 (2H, t, J = 6.6 Hz), 3.02 (3H, s), 3.21 (2H, t, J = 6.6 Hz), 3.49-3.58 (2H, m), 3.82 (3H, |
| | s), 4.06 (2H, t, J = 6.6 Hz), 4.37 (2H, d, J = 6.1 Hz), 6.75-6.82 (1H, m), 7.31 (1H, d, J = 8.1 |
| | Hz), 7.43-7.56 (3H, m), 7.58-7.73 (2H, m), 7.77-7.85 (2H, m), 8.10-8.16 (1H, m), 9.04 |
| | (1H, t, J = 6.2 Hz) |
| 1236 | 2.36 (3H, s), 2.95 (2H, t, J = 6.5 Hz), 3.22 (2H, t, J = 6.6 Hz), 3.53-3.59 (4H, m), 3.61 (2H, t, |
| | J = 6.6 Hz), 4.06 (2H, t, J = 6.6 Hz), 4.48 (2H, d, J = 6.0 Hz), 4.78 (1H, t, J = 5.4 Hz), 7.19 (1H, |
| | d, J = 4.9 Hz), 7.32 (1H, d, J = 8.2 Hz), 7.45 (1H, dd, J = 8.1, 2.3 Hz), 7.48-7.55 (2H, m), |
| | 7.62-7.69 (1H, m), 7.79-7.85 (2H, m), 8.32 (1H, d, J = 4.9 Hz), 8.43 (1H, s), 9.01 (1H, t, J = |
| | 6.0 Hz) |
| 1237 | 0.54-0.66 (2H, m), 0.85 (2H, d, J = 8.0 Hz), 1.38 (2H, s), 1.48 (4H, p, J = 5.5 Hz), 1.84 (1H, |
| | td, J = 8.4, 4.2 Hz), 2.22-2.41 (4H, m), 3.20 (2H, t, J = 6.6 Hz), 3.42 (3H, s), 4.06 (2H, t, J = |
| | 6.6 Hz), 4.35 (2H, d, J = 6.0 Hz), 7.17 (1H, d, J = 7.5 Hz), 7.21-7.29 (2H, m), 7.33 (1H, t, J = |
| | 7.7 Hz), 7.48-7.55 (2H, m), 7.63 (1H, dt, J = 6.9, 2.1 Hz), 7.79 (1H, d, J = 2.1 Hz), 8.74 (1H, |
| | s), 12.26 (1H, s) |
| 1238 | 1.93-2.02 (2H, m), 2.19 (3H, d, J = 0.7 Hz), 2.69 (2H, t, J = 7.1 Hz), 3.05 (3H, s), 3.16 (2H, t, |
| | J = 6.3 Hz), 3.21 (2H, t, J = 6.6 Hz), 3.64 (3H, s), 4.07 (2H, t, J = 6.6 Hz), 4.31(2H, d, J = 6.0 |
| | Hz), 5.93 (1H, d, J = 0.8 Hz), 7.26 (1H, d, J = 8.2 Hz), 7.39 (1H, dd, J = 8.1, 2.4 Hz), 7.48 (1H, |
| | d, J = 2.2 Hz), 7.49-7.54 (2H, m), 7.61-7.67 (1H, m), 7.76-7.82 (1H, m), 8.68 (1H, t, J = |
| | 6.1 Hz). |
| 1239 | 0.15-0.31 (2H, m), 0.36-0.49 (2H, m), 0.95-1.12 (1H, m), 1.90-2.07 (2H, m), 2.69 (2H, |
| | t, J = 7.1 Hz), 3.03-3.09 (3H, m), 3.09-3.19 (4H, m), 3.19-3.25 (2H, m), 4.07 (2H, t, J = |
| | 6.5 Hz), 7.26 (1H, d, J = 8.1 Hz), 7.34-7.44 (1H, m), 7.46-7.58 (3H, m), 7.59-7.68 (1H, |
| | m), 7.77-7.83 (1H, m), 8.52 (1H, t, J = 6.0 Hz). |
| 1240 | 2.36 (3H, s), 2.90 (2H, t, J = 6.6 Hz), 3.22 (2H, t, J = 6.6 Hz), 3.36-3.41 (2H, m), 4.07 (2H, t, |
| | J = 6.6 Hz), 4.48 (2H, d, J = 6.0 Hz), 7.19 (1H, d, J = 4.9 Hz), 7.34 (1H, d, J = 8.2 Hz), 7.44- |
| | 7.48 (1H, m), 7.49-7.55 (2H, m), 7.60-7.68 (1H, m), 7.78-7.84 (2H, m), 7.96-8.03 (1H, |
| | m), 8.32 (1H, d, J = 4.9 Hz), 8.44 (1H, s), 9.01 (1H, t, J = 6.0 Hz) |
| 1241 | 2.01-2.10 (2H, m), 2.15 (2H, t, J = 7.1 Hz), 2.19 (3H, d, J = 0.7 Hz), 2.64 (2H, t, J = 7.0 Hz), |
| | 3.19-3.24 (5H, m), 3.64 (3H, s), 4.09 (2H, t, J = 6.6 Hz), 4.31 (2H, d, J = 6.1 Hz), 5.94 (1H, d, |
| | J = 0.9 Hz), 7.18 (1H, dd, J = 8.1, 2.1 Hz), 7.28 (1H, d, J = 8.2 Hz), 7.34 (1H, d, J = 2.1 Hz), |
| | 7.46-7.57 (2H, m), 7.60-7.67 (1H, m), 7.77-7.82 (1H, m), 8.70 (1H, t, J = 6.1 Hz) |
| 1242 | 0.19-0.28 (2H, m), 0.38-0.46 (2H, m), 0.99-1.12 (1H, m), 2.01-2.09 (2H, m), 2.15 (2H, |
| | t, J = 7.0 Hz), 2.60-2.69 (2H, m), 3.12 (2H, t, J = 6.4 Hz), 3.19-3.25 (5H, m), 4.09 (2H, t, |
| | J = 6.6 Hz), 7.18 (1H, dd, J = 8.1, 2.1 Hz), 7.28 (1H, d, J = 8.1 Hz), 7.34 (1H, d, J = 2.1 Hz), |
| | 7.48-7.58 (2H, m), 7.60-7.66 (1H, m), 7.76-7.81 (1H, m), 8.53 (1H, t, J = 6.0 Hz) |
| 1243 | 0.17-0.28 (2H, m), 0.37-0.45 (2H, m), 1.01-1.08 (1H, m), 1.77-1.97 (1H, m), 2.04- |
| | 2.23 (1H, m), 2.25-2.32 (1H, m), 2.54-2.65 (1H, m), 2.71-2.83 (2H, m), 3.11 (2H, t, J = |
| | 6.5 Hz), 3.20 (2H, t, J = 6.6 Hz), 3.54-3.64 (2H, m), 4.07 (2H, t, J = 6.6 Hz), 5.09-5.29 (1H, |
| | m), 7.19 (1H, d, J = 7.6 Hz), 7.23-7.27 (1H, m), 7.29 (1H, d, J = 1.9 Hz), 7.34 (1H, t, J = 7.8 |
| | Hz), 7.47-7.56 (2H, m), 7.63 (1H, dt, J = 6.9, 2.0 Hz), 7.77-7.81 (1H, m), 8.54 (1H, t, J = |
| | 6.0 Hz) |
| 1244 | 0.19-0.27 (2H, m), 0.35-0.48 (2H, m), 0.96-1.11 (1H, m), 2.17 (3H, s), 2.76 (3H, s), 2.99 |
| | (3H, s), 3.11 (2H, t, J = 6.4 Hz), 3.20 (2H, t, J = 6.6 Hz), 4.05 (2H, t, J = 6.5 Hz), 7.17 |
| | (1H, d, J = 2.0 Hz), 7.24-7.34 (2H, m), 7.44-7.57 (2H, m), 7.63 (1H, dt, J = 6.8, 2.1 Hz), 7.76-7.82 |
| | (1H, m), 8.54 (1H, t, J = 6.1 Hz) |
| 1245 | 2.97 (2H, t, J = 6.6 Hz), 3.02 (3H, s), 3.22 (2H, t, J = 6.6 Hz), 3.54 (2H, t, J = 6.6 Hz), 4.06 |
| | (2H, t, J = 6.5 Hz), 4.42 (2H, d, J = 6.0 Hz), 6.17 (1H, s), 7.31 (1H, d, J = 8.2 Hz), 7.45 (1H, |
| | dd, J = 8.1, 2.3 Hz), 7.50 (2H, d, J = 6.5 Hz), 7.61-7.66 (2H, m), 7.80 (1H, d, J = 2.1 Hz), |
| | 7.82 (1H, d, J = 2.4 Hz), 8.75 (1H, t, J = 6.1 Hz), 12.61 (1H, d, J = 33.3 Hz) |
| 1246 | 2.96 (2H, t, J = 6.6 Hz), 3.02 (3H, s), 3.16-3.26 (4H, m), 3.26-3.30 (1H, m), 3.40-3.48 |
| | (1H, m), 3.51-3.58 (3H, m), 3.59-3.75 (4H, m), 4.05 (2H, t, J = 6.6 Hz), 7.31 (1H, d, J = 8.2 |
| | Hz), 7.44 (1H, dd, J = 8.1, 2.4 Hz), 7.49-7.53 (2H, m), 7.60-7.66 (1H, m), 7.79 (1H, m), |
| | 7.82 (1H, d, J = 2.4 Hz), 8.43 (1H, t, J = 6.0 Hz) |
| 1247 | 2.27 (3H, s), 2.97 (2H, t, J = 6.6 Hz), 3.02 (3H, s), 3.18-3.25 (2H, m), 3.51-3.57 (2H, m), |
| | 4.06 (2H, t, J = 6.6 Hz), 4.43 (2H, d, J = 5.9 Hz), 7.31 (1H, d, J = 8.2 Hz), 7.45 (1H, dd, J = |
| | 8.1, 2.4 Hz), 7.49-7.52 (2H, m), 7.65 (1H, ddd, J = 6.1, 2.8, 2.0 Hz), 7.81 (1H, dt, J = 2.1, |
| | 1.0 Hz), 7.83 (1H, d, J = 2.3 Hz), 8.74 (1H, br, s), 13.36 (1H, br, s) |
| 1248 | 2.01 (3H, d, J = 0.7 Hz), 2.98 (2H, t, J = 6.6 Hz), 3.04 (3H, s), 3.23 (2H, t, J = 6.5 Hz), 3.56 |
| | (2H, t, J = 6.7 Hz), 4.07 (2H, t, J = 6.6 Hz), 4.43 (2H, d, J = 5.8 Hz), 7.33 (1H, d, J = 8.1 Hz), |
| | 7.46 (1H, dd, J = 8.1, 2.3 Hz), 7.49-7.59 (2H, m), 7.63-7.68 (1H, m), 7.82 (2H, dd, J = |
| | 11.7, 2.2 Hz), 8.14 (1H, ddt, J = 29.5, 8.4, 1.0 Hz), 8.52 (1H, br, s), 12.35 (1H, s) |
| 1249 | 2.98 (2H, t, J = 6.6 Hz), 3.03 (3H, s), 3.18-3.32 (5H, m), 3.42-3.50 (1H, m), 3.51-3.59 |
| | (3H, m), 3.60-3.78 (4H, m), 4.07 (2H, t, J = 6.6 Hz), 7.32 (1H, d, J = 8.2 Hz), 7.45 (1H, dd, J = |
| | 8.0, 2.4 Hz), 7.50-7.55 (2H, m), 7.65 (1H, dt, J = 6.5, 2.2 Hz), 7.81 (1H, td, J = 1.9, 0.7 |
| | Hz), 7.83 (1H, d, J = 2.3 Hz), 8.44 (1H, t, J = 6.1 Hz) |
| 1250 | 0.29-0.39 (2H, m), 0.43-0.53 (2H, m), 2.98 (2H, t, J = 6.6 Hz), 3.04 (3H, s), 3.22 (2H, t, J = |
| | 6.6 Hz), 3.31 (4H, t, J = 5.5 Hz), 3.55 (2H, t, J = 6.7 Hz), 4.07 (2H, t, J = 6.6 Hz), 4.67 (1H, t, |
| | J = 5.8 Hz), 7.33 (1H, d, J = 8.2 Hz), 7.46 (1H, dd, J = 8.1, 2.4 Hz), 7.49-7.56 (2H, m), 7.64 |
| | (1H, dt, J = 6.5, 2.2 Hz), 7.80 (1H, td, J = 1.9, 0.7 Hz), 7.84 (1H, d, J = 2.3 Hz), 8.41 (1H, t, J = |
| | 6.0 Hz) |
| 1251 | 2.98 (2H, t, J = 6.6 Hz), 3.03 (3H, s), 3.19-3.25 (2H, m), 3.45-3.61 (4H, m), 3.65 (1H, dd, J = |
| | 8.8, 5.3 Hz), 3.71 (1H, dd, J = 8.6, 7.1 Hz), 3.87 (1H, dd, J = 8.5, 7.7 Hz), 3.94 (1H, dd, J = |
| | 8.8, 6.5 Hz), 4.03-4.10 (2H, m), 4.59 (1H, qd, J = 7.6, 5.4 Hz), 4.87 (1H, dd, J = 5.1, 4.4 Hz), |
| | 7.32 (1H, d, J = 8.1 Hz), 7.45 (1H, dd, J = 8.1, 2.4 Hz), 7.50-7.55 (2H, m), 7.64 (1H, dt, J = |
| | 6.7, 2.2 Hz), 7.80 (1H, td, J = 1.9, 0.7 Hz), 7.83 (1H, d, J = 2.3 Hz), 8.55 (1H, d, J = 7.6 Hz) |
| | 1 x H not seen |
| 1252 | 2.98 (2H, t, J = 6.6 Hz), 3.03 (3H, s), 3.23 (2H, t, J = 6.6 Hz), 3.50-3.57 (4H, m), 3.60 (2H, d, |
| | J = 5.6 Hz), 4.07 (2H, t, J = 6.6 Hz), 4.28 (2H, d, J = 5.9 Hz), 4.41 (2H, d, J = 5.9 Hz), 4.95 |
| | (1H, t, J = 5.6 Hz), 7.32 (1H, d, J = 8.1 Hz), 7.45 (1H, dd, J = 8.0, 2.4 Hz), 7.49-7.56 (2H, m), |
| | 7.64 (1H, dt, J = 6.7, 2.1 Hz), 7.80 (1H, td, J = 1.9, 0.7 Hz), 7.83 (1H, d, J = 2.3 Hz), 8.65 (1H, |
| | t, J = 6.3 Hz) |
| 1253 | 0.21-0.26 (2H, m), 0.39-0.44 (2H, m), 1.00-1.10 (1H, m), 2.98 (2H, t, J = 6.5 Hz), 3.12 |
| | (2H, t, J = 6.5 Hz), 3.22 (2H, t, J = 6.6 Hz), 3.62 (2H, t, J = 6.5 Hz), 3.80 (2H, dt, J = 26.8, 5.0 Hz), |
| | 4.07 (2H, t, J = 6.6 Hz), 4.62 (2H, dt, J = 47.5, 5.0 Hz), 7.34 (1H, d, J = 8.2 Hz), 7.48 (1H, dd, |
| | J = 8.0, 2.3 Hz), 7.50-7.56 (2H, m), 7.62-7.67 (1H, m), 7.79-7.82 (1H, m), 7.84 (1H, d, |
| | J = 2.3 Hz), 8.53 (1H, t, J = 6.0 Hz). |
| 1254 | 2.19 (3H, d, J = 0.7 Hz), 2.98 (2H, t, J = 6.5 Hz), 3.22 (2H, t, J = 6.6 Hz), 3.59-3.67 (5H, m), |
| | 3.80 (2H, dt, J = 26.7, 5.0 Hz), 4.07 (2H, t, J = 6.6 Hz), 4.31 (2H, d, J = 6.1 Hz), 4.62 (2H, dt, |
| | J = 47.4, 5.0 Hz), 5.94 (1H, d, J = 0.8 Hz), 7.34 (1H, d, J = 8.1 Hz), 7.45-7.54 (3H, m), 7.61- |
| | 7.68 (1H, m), 7.79-7.82 (1H, m), 7.84 (1H, d, J = 2.3 Hz), 8.70 (1H, t, J = 6.1 Hz). |
| 1255 | 2.16 (3H, s), 2.95 (2H, t, J = 6.5 Hz), 3.22 (2H, t, J = 6.6 Hz), 3.53-3.65 (6H, m), 4.07 (2H, t, |
| | J = 6.6 Hz), 4.36 (2H, d, J = 6.0 Hz), 4.74-4.81 (1H, m), 5.91 (1H, d, J = 0.7 Hz), 7.32 (1H, d, |
| | J = 8.2 Hz), 7.45 (1H, dd, J = 8.1, 2.3 Hz), 7.48-7.53 (2H, m), 7.62-7.68 (1H, m), 7.79-7.82 |
| | (1H, m), 7.83 (1H, d, J = 2.4 Hz), 8.71 (1H, s), 12.21 (1H, s). |
| 1256 | 2.31 (3H, s), 2.38 (3H, s), 2.95 (2H, t, J = 6.5 Hz), 3.21 (2H, t, J = 6.6 Hz), 3.51-3.63 (6H, m), |
| | 4.06 (2H, t, J = 6.6 Hz), 4.43 (2H, d, J = 5.9 Hz), 4.73-4.82 (1H, m), 7.05 (1H, d, J = 0.8 Hz), |
| | 7.32 (1H, d, J = 8.1 Hz), 7.45 (1H, dd, J = 8.1, 2.3 Hz), 7.47-7.54 (2H, m), 7.61-7.66 (1H, m), |
| | 7.78-7.81 (1H, m), 7.82 (1H, d, J = 2.3 Hz), 8.30 (1H, s), 8.94 (1H, t, J = 6.0 Hz). |
| 1257 | 0.20-0.28 (2H, m), 0.36-0.48 (2H, m), 0.98-1.12 (1H, m), 1.51-1.64 (1H, m), 1.86- |
| | 1.99 (1H, m), 2.51-2.66 (1H, m), 2.96 (2H, t, J = 6.5 Hz), 3.12 (2H, t, J = 6.4 Hz), 3.18-3.26 |
| | (2H, m), 3.38-3.51 (2H, m), 3.49-3.67 (4H, m), 3.66-3.81 (2H, m), 4.06 (2H, t, J = 6.6 |
| | Hz), 7.33 (1H, d, J = 8.0 Hz), 7.46 (1H, dd, J = 8.1, 2.3 Hz), 7.46-7.57 (2H, m), 7.59-7.70 |
| | (1H, m), 7.77-7.86 (2H, m), 8.52 (1H, t, J = 6.0 Hz) |
| 1258 | 1.51-1.64 (1H, m), 1.86-1.99 (1H, m), 2.15-2.21 (3H, m), 2.53-2.67 (1H, m), 2.96 (2H, |
| | t, J = 6.5 Hz), 3.22 (2H, t, J = 6.6 Hz), 3.38-3.62 (6H, m), 3.65 (3H, s), 3.67-3.82 (2H, m), |
| | 4.06 (2H, t, J = 6.6 Hz), 4.31 (2H, d, J = 6.1 Hz), 5.94 (1H, d, J = 0.8 Hz), 7.33 (1H, d, J = 8.2 |
| | Hz), 7.42-7.50 (1H, m), 7.45-7.56 (2H, m), 7.60-7.70 (1H, m), 7.78-7.86 (2H, m), 8.69 |
| | (1H, t, J = 6.1 Hz) |
| 1259 | 0.53-0.68 (2H, m), 0.78-0.89 (2H, m), 1.75-1.89 (1H, m), 2.95 (2H, t, J = 6.5 Hz), 3.22 |
| | (2H, t, J = 6.6 Hz), 3.49-3.65 (6H, m), 4.07 (2H, t, J = 6.6 Hz), 4.35 (2H, d, J = 6.0 Hz), 4.77 |
| | (1H, s), 5.82 (1H, br.s), 7.32 (1H, d, J = 8.1 Hz), 7.42-7.49 (1H, m), 7.49-7.55 (2H, m), |
| | 7.59-7.69 (1H, m), 7.79-7.82 (1H, m), 7.83 (1H, d, J = 2.3 Hz), 8.72 (1H, t, J = 6.0 Hz), |
| | 12.14 (1H, br. s) |
| 1260 | 1.51-1.67 (1H, m), 1.79-1.98 (1H, m), 2.95 (2H, t, J = 6.5 Hz), 3.19-3.27 (4H, m), 3.43- |
| | 3.78 (11H, m), 4.06 (2H, t, J = 6.6 Hz), 4.71-4.81 (1H, m), 7.32 (1H, d, J = 8.2 Hz), 7.45 |
| | (1H, dd, J = 8.1, 2.3 Hz), 7.48-7.55 (2H, m), 7.59-7.67 (1H, m), 7.78-7.80 (1H, m), 7.82 |
| | (1H, d, J = 2.3 Hz), 8.62 (1H, t, J = 6.1 Hz) |
| 1261 | 0.21-0.27 (2H, m), 0.37-0.46 (2H, m), 0.98-1.10 (1H, m), 2.94 (2H, t, J = 6.5 Hz), 3.12 |
| | (2H, t, J = 6.4 Hz), 3.22 (2H, t, J = 6.6 Hz), 3.25 (3H, s), 3.51 (2H, t, J = 5.6 Hz), 3.55-3.62 |
| | (2H, m), 3.65 (2H, t, J = 5.6 Hz), 4.07 (2H, t, J = 6.6 Hz), 7.32 (1.4.4H, d, J = 8.1 Hz), 7.46 |
| | (1H, dd, J = 8.1, 2.3 Hz), 7.48-7.56 (2H, m), 7.60-7.68 (1H, m), 7.79-7.81 (1H, m), 7.83 |
| | (1H, d, J = 2.3 Hz), 8.52 (1H, t, J = 6.1 Hz). |
| 1262 | 2.16-2.21 (3H, m), 2.94 (2H, t, J = 6.5 Hz), 3.22 (2H, t, J = 6.5 Hz), 3.25 (3H, s), 3.51 (2H, t, |
| | J = 5.6 Hz), 3.59 (2H, t, J = 6.6 Hz), 3.62-3.67 (5H, m), 4.07 (2H, t, J = 6.6 Hz), 4.31 (2H, d, |
| | J = 6.1 Hz), 5.90-5.95 (1H, m), 7.32 (1H, d, J = 8.2 Hz), 7.42-7.49 (1H, m), 7.49-7.54 (2H, |
| | m), 7.61-7.68 (1H, m), 7.79-7.82 (1H, m), 7.83 (1H, d, J = 2.3 Hz), 8.70 (1H, t, J = 6.1 Hz). |
| 1263 | 0.20-0.28 (2H, m), 0.36-0.47 (2H, m), 0.98-1.12 (1H, m), 1.77-1.92 (4H, m), 3.12 (2H, |
| | t, J = 6.5 Hz), 3.22 (2H, t, J = 6.6 Hz), 3.38-3.49 (4H, m), 4.09 (2H, t, J = 6.6 Hz), 7.34-7.42 |
| | (1H, m), 7.42-7.48 (2H, m), 7.48-7.59 (3H, m), 7.65 (1H, dt, J = 6.7, 2.1 Hz), 7.78-7.84 |
| | (1H, m), 8.54 (1H, t, J = 6.0 Hz) |
| 1264 | 2.08 (3H, s), 2.98 (2H, t, J = 6.6 Hz), 3.03 (3H, s), 3.22 (2H, t, J = 6.6 Hz), 3.55 (2H, t, J = 6.7 |
| | Hz), 3.74 (3H, s), 4.07 (2H, t, J = 6.6 Hz), 4.42 (2H, d, J = 6.0 Hz), 5.93 (1H, s), 7.32 (1H, d, |
| | J = 8.2 Hz), 7.45 (1H, dd, J = 8.1, 2.3 Hz), 7.48-7.55 (2H, m), 7.64 (1H, dt, J = 6.7, 2.1 Hz), |
| | 7.80 (1H, td, J = 1.9, 0.6 Hz), 7.83 (1H, d, J = 2.3 Hz), 8.97 (1H, t, J = 6.0 Hz) |
| 1265 | 1.64-1.81 (2H, m), 2.95-3.02 (3H, m), 3.03 (4H, s), 3.22 (2H, td, J = 6.4, 1.9 Hz), 3.31 (3H, |
| | s), 3.41 (1H, td, J = 9.8, 4.7 Hz), 3.55 (2H, t, J = 6.6 Hz), 3.75-3.84 (1H, m), 3.97 (1H, ddd, |
| | J = 13.3, 5.5, 3.1 Hz), 4.02-4.11 (3H, m), 7.32 (1H, d, J = 8.2 Hz), 7.45 (1H, dd, J = 8.1, 2.3 |
| | Hz), 7.49-7.55 (2H, m), 7.64 (1H, dt, J = 6.7, 2.2 Hz), 7.80 (1H, td, J = 1.9, 0.7 Hz), 7.83 |
| | (1H, d, J = 2.3 Hz), 8.46 (1H, d, J = 8.8 Hz) |
| 1266 | 1.43 (3H, s), 1.61 (2H, m), 2.21-2.28 (2H, m), 2.98 (2H, t, J = 6.6 Hz), 3.04 (3H, s), 3.17- |
| | 3.25 (2H, m), 3.52-3.60 (4H, m), 3.60-3.69 (2H, m), 4.07 (2H, t, J = 6.6 Hz), 7.33 (1H, d, |
| | J = 8.1 Hz), 7.46 (1H, dd, J = 8.1, 2.4 Hz), 7.50-7.56 (3H, m), 7.64-7.68 (1H, m), 7.82 (1H, |
| | td, J = 1.8, 0.8 Hz), 7.84 (1H, d, J = 2.3 Hz) |
| 1267 | 2.59 (3H, s), 2.97 (2H, t, J = 6.7 Hz), 3.02 (3H, s), 3.21 (2H, t, J = 6.6 Hz), 3.54 (2H, t, J = 6.6 |
| | Hz), 4.06 (2H, t, J = 6.5 Hz), 4.70 (2H, d, J = 6.1 Hz), 7.31 (1H, d, J = 8.1 Hz), 7.45 (1H, |
| | dd, J = 8.1, 2.4 Hz), 7.48-7.58 (4H, m), 7.63-7.68 (1H, m), 7.80-7.85 (2H, m), 9.17 (1H, t, J = |
| | 6.1 Hz) |
| 1268 | 2.08-2.23 (6H, m), 2.96 (2H, t, J = 6.6 Hz), 3.02 (3H, s), 3.20 (2H, t, J = 6.6 Hz), 3.54 (2H, t, |
| | J = 6.7 Hz), 4.05 (2H, t, J = 6.6 Hz), 4.19 (2H, d, J = 5.8 Hz), 7.30 (1H, d, J = 8.1 Hz), 7.40- |
| | 7.46 (1H, m), 7.46-7.52 (2H, m), 7.61 (1H, dt, J = 6.8, 2.1 Hz), 7.77 (1H, td, J = 1.9, 0.7 Hz), |
| | 7.81 (1H, d, J = 2.4 Hz), 8.54 (1H, t, J = 5.8 Hz), 11.98 (1H, s) |
| 1269 | 0.18-0.29 (2H, m), 0.36-0.46 (2H, m), 0.97-1.12 (1H, m), 1.80 (3H, s), 3.05-3.19 (5H, |
| | m), 3.22 (2H, t, J = 6.6 Hz), 4.10 (2H, t, J = 6.6 Hz), 7.22 (1H, d, J = 7.8 Hz), 7.30-7.48 (3H, |
| | m), 7.48-7.59 (2H, m), 7.64 (1H, dt, J = 6.8, 2.1 Hz), 7.77-7.83 (1H, m), 8.54 (1H, t, J = |
| | 6.0 Hz) |
| 1270 | 0.18-0.30 (2H, m), 0.34-0.47 (4H, m), 0.54 (2H, s), 1.01-1.10 (1H, m), 2.93 (4H, d, J = |
| | 23.2 Hz), 3.12 (2H, t, J = 6.5 Hz), 3.22 (2H, t, J = 6.6 Hz), 4.08 (2H, t, J = 6.6 Hz), 7.37 (1H, |
| | s), 7.41-7.45 (2H, m), 7.48-7.55 (3H, m), 7.64 (1H, dt, J = 6.6, 2.2 Hz), 7.80 (1H, t, J = 1.6 |
| | Hz), 8.53 (1H, t, J = 6.0 Hz) |
| 1271 | 2.97 (2H, t, J = 6.7 Hz), 3.02 (3H, s), 3.21 (2H, t, J = 6.6 Hz), 3.54 (2H, t, J = 6.7 Hz), 3.88 |
| | (3H, s), 4.06 (2H, t, J = 6.6 Hz), 4.43 (2H, d, J = 6.0 Hz), 7.04 (1H, d, J = 5.7 Hz), 7.31 (1H, d, |
| | J = 8.2 Hz), 7.44 (1H, dd, J = 8.0, 2.4 Hz), 7.48-7.54 (2H, m), 7.62-7.68 (1H, m), 7.79- |
| | 7.83 (2H, m), 8.26 (1H, s), 8.36 (1H, d, J = 5.7 Hz), 8.85 (1H, t, J = 6.1 Hz) |
| 1272 | 2.97 (2H, t, J = 6.6 Hz), 3.02 (3H, s), 3.11 (2H, t, J = 6.6 Hz), 3.54 (2H, t, J = 6.5 Hz), 3.62 |
| | (2H, d, J = 4.9 Hz), 3.66 (4H, s), 3.93 (2H, t, J = 4.9 Hz), 4.06 (2H, t, J = 6.6 Hz), 7.31 (1H, d, |
| | J = 8.2 Hz), 7.44 (1H, dd, J = 8.1, 2.4 Hz), 7.50 (2H, dd, J = 5.0, 1.9 Hz), 7.57-7.62 (1H, m), |
| | 7.76 (1H, q, J = 1.5 Hz), 7.82 (1H, d, J = 2.4 Hz) |
| 1273 | 0.20-0.28 (2H, m), 0.37-0.46 (2H, m), 0.97-1.12 (1H, m), 2.86 (3H, s), 2.96 (3H, s), 3.12 |
| | (2H, t, J = 6.5 Hz), 3.19 (2H, t, J = 6.5 Hz), 4.38 (2H, t, J = 6.4 Hz), 7.22 (1H, dd, J = 5.0, 1.4 |
| | Hz), 7.48-7.60 (2H, m), 7.61-7.72 (2H, m), 7.81-7.86 (1H, m), 8.47-8.59 (2H, m) |
| 1274 | 1.86 (2H, m), 1.98-2.08 (2H, m), 2.26-2.35 (1H, m), 2.96 (2H, t, J = 6.6 Hz), 3.02 (3H, s), |
| | 3.17-3.23 (2H, m), 3.25-3.31 (2H, m), 3.51-3.57 (2H, m), 4.05 (2H, t, J = 6.6 Hz), 4.15- |
| | 4.25 (1H, m), 4.93 (1H, dd, J = 6.4, 4.2 Hz), 7.31 (1H, d, J = 8.2 Hz), 7.44 (1H, dd, J = 8.1, |
| | 2.4 Hz), 7.48-7.53 (2H, m), 7.60-7.64 (1H, m), 7.78 (1H, td, J = 1.8, 0.7 Hz), 7.82 (1H, d, |
| | J = 2.4 Hz), 8.47 (1H, t, J = 6.1 Hz) |
| 1275 | 1.70 (2H, p, J = 6.6 Hz), 2.19 (3H, s), 2.96 (2H, t, J = 6.6 Hz), 3.22 (2H, t, J = 6.6 Hz), 3.40- |
| | 3.48 (2H, m), 3.48-3.58 (4H, m), 3.65 (3H, s), 4.06 (2H, t, J = 6.6 Hz), 4.31 (2H, d, J = 6.0 |
| | Hz), 4.48 (1H, t, J = 5.2 Hz), 5.83-5.99 (1H, m), 7.32 (1H, d, J = 8.2 Hz), 7.43-7.48 (1H, m), |
| | 7.49-7.54 (2H, m), 7.60-7.67 (1H, m), 7.79-7.82 (1H, m), 7.83 (1H, d, J = 2.3 Hz), 8.67 |
| | (1H, t, J = 6.1 Hz) |
| 1276 | 1.27 (6H, s), 2.19 (3H, s), 3.11 (3H, s), 3.22 (2H, t, J = 6.6 Hz), 3.65 (3H, s), 4.08 (2H, t, J = |
| | 6.6 Hz), 4.31 (2H, d, J = 6.2 Hz), 5.94 (1H, s), 7.00-7.06 (2H, m), 7.37 (1H, d, J = 7.8 Hz), |
| | 7.47-7.54 (2H, m), 7.59-7.66 (1H, m), 7.77-7.82 (1H, m), 8.69 (1H, t, J = 6.1 Hz) |
| 1277 | 0.19-0.26 (2H, m), 0.39-0.45 (2H, m), 0.99-1.11 (1H, m), 1.27 (6H, s), 3.09-3.15 (5H, |
| | m), 3.22 (2H, t, J = 6.6 Hz), 4.08 (2H, t, J = 6.6 Hz), 7.00-7.06 (2H, m), 7.37 (1H, d, J = 7.8 |
| | Hz), 7.48-7.56 (2H, m), 7.60-7.66 (1H, m), 7.79 (1H, t, J = 2.1 Hz), 8.52 (1H, t, J = 6.0 Hz) |
| 1278 | 0.20-0.28 (2H, m), 0.36-0.46 (2H, m), 0.97-1.12 (1H, m), 2.95 (3H, s), 3.00 (3H, s), 3.12 |
| | (2H, t, J = 6.4 Hz), 3.23 (2H, t, J = 6.5 Hz), 4.18 (2H, t, J = 6.5 Hz), 7.47-7.60 (4H, m), 7.62- |
| | 7.72 (1H, m), 7.79-7.84 (1H, m), 8.51-8.59 (2H, m) |
| 1279 | 0.18-0.32 (2H, m), 0.37-0.50 (2H, m), 0.97-1.11 (1H, m), 2.94 (3H, s), 3.00 (3H, s), 3.12 |
| | (2H, t, J = 6.4 Hz), 3.24 (2H, t, J = 6.6 Hz), 4.14 (2H, t, J = 6.6 Hz), 7.48-7.58 (2H, m), 7.65 |
| | (1H, dt, J = 6.6, 2.2 Hz), 7.79-7.84 (1H, m), 7.88 (1H, t, J = 2.2 Hz), 8.47 (1H, d, J = 1.9 Hz), |
| | 8.55 (1H, t, J = 6.0 Hz), 8.69 (1H, d, J = 2.4 Hz) |
| 1280 | 1.37 (2H, dd, J = 4.5, 1.7 Hz), 1.61 (2H, dd, J = 4.6, 1.7 Hz), 2.98 (2H, t, J = 6.6 Hz), 3.03 |
| | (3H, s), 3.21 (2H, t, J = 6.6 Hz), 3.50-3.60 (8H, m), 4.07 (2H, t, J = 6.6 Hz), 4.65 (1H, t, J = |
| | 5.9 Hz), 7.32 (1H, d, J = 8.1 Hz), 7.45 (1H, dd, J = 8.1, 2.4 Hz), 7.49-7.55 (2H, m), 7.61- |
| | 7.67 (1H, m), 7.77-7.85 (2H, m), 8.63 (1H, t, J = 6.3 Hz) |
| 1281 | 0.18-0.30 (2H, m), 0.35-0.50 (2H, m), 0.97-1.13 (1H, m), 2.86 (3H, d, J = 1.2 Hz), 3.00 |
| | (3H, s), 3.12 (2H, t, J = 6.5 Hz), 3.22 (2H, t, J = 6.6 Hz), 4.06 (2H, t, J = 6.6 Hz), 7.34 (1H, |
| | t, J = 9.0 Hz), 7.41 (1H, dd, J = 6.0, 2.7 Hz), 7.45-7.57 (3H, m), 7.64 (1H, dt, J = 6.6, 2.2 Hz), |
| | 7.80 (1H, td, J = 1.9, 0.7 Hz), 8.53 (1H, t, J = 6.0 Hz) |
| 1282 | 2.32 (3H, t, J = 0.7 Hz), 2.98 (2H, t, J = 6.9 Hz), 3.03 (3H, s), 3.20-3.27 (2H, m), 3.55 (2H, |
| | dd, J = 7.2, 6.2 Hz), 4.07 (2H, t, J = 6.6 Hz), 4.72 (2H, d, J = 6.1 Hz), 7.32 (1H, d, J = 8.1 Hz), |
| | 7.43-7.48 (2H, m), 7.49-7.56 (2H, m), 7.64-7.70 (1H, m), 7.81-7.85 (2H, m), 9.01 (1H, |
| | dd, J = 2.1, 0.7 Hz), 9.17 (1H, t, J = 6.1 Hz) |
| 1283 | 1.57 (2H, dd, J = 7.1, 2.6 Hz), 1.73-1.85 (4H, m), 2.98 (2H, t, J = 6.6 Hz), 3.03 (3H, s), 3.21 |
| | (4H, dd, J = 7.9, 5.9 Hz), 3.29 (2H, d, J = 6.3 Hz), 3.55 (2H, t, J = 6.7 Hz), 3.97 (2H, t, J = 6.8 |
| | Hz), 4.07 (2H, t, J = 6.6 Hz), 4.55 (1H, t, J = 5.5 Hz), 7.32 (1H, d, J = 8.1 Hz), 7.45 (1H, dd, J = |
| | 8.1, 2.4 Hz), 7.50-7.55 (2H, m), 7.63-7.67 (1H, m), 7.80-7.82 (1H, m), 7.83 (1H, d, J = |
| | 2.4 Hz), 8.16 (1H, t, J = 6.2 Hz) |
| 1284 | (CDCl3) 1.44 (3H, t, J = 7.1 Hz), 2.99 (2H, t, J = 6.6 Hz), 3.15 (3H, s), 3.33 (2H, t, J = 6.7 Hz), |
| | 3.55 (2H, t, J = 6.7 Hz), 4.15 (2H, t, J = 6.7 Hz), 4.47 (2H, q, J = 7.1 Hz), 7.17 (1H, d, J = 8.1 |
| | Hz), 7.29-7.38 (2H, m), 7.41-7.52 (2H, m), 7.62 (1H, t, J = 1.9 Hz), 7.97 (1H, d, J = 2.3 Hz) |
| 1285 | 0.17-0.30 (2H, m), 0.30-0.52 (2H, m), 1.05 (1H, td, J = 8.7, 4.6 Hz), 1.84 (4H, q, J = 9.9 |
| | Hz), 2.38 (2H, t, J = 6.2 Hz), 3.12 (2H, t, J = 6.5 Hz), 3.21 (2H, t, J = 6.6 Hz), 3.59 (2H, t, J = |
| | 5.6 Hz), 4.08 (2H, t, J = 6.6 Hz), 7.10-7.19 (1H, m), 7.20-7.28 (1H, m), 7.30 (1H, t, J = 2.1 |
| | Hz), 7.39 (1H, t, J = 8.0 Hz), 7.47-7.58 (2H, m), 7.63 (1H, dt, J = 7.0, 2.0 Hz), 7.79 (1H, t, |
| | J = 2.0 Hz), 8.54 (1H, t, J = 6.0 Hz) |
| 1286 | 0.20-0.28 (2H, m), 0.38-0.46 (2H, m), 1.02-1.19 (7H, m), 3.12 (2H, t, J = 6.5 Hz), 3.22 |
| | (4H, t, J = 6.6 Hz), 3.38-3.46 (2H, m), 4.08 (2H, t, J = 6.6 Hz), 7.22 (1H, dt, J = 6.9, 1.7 Hz), |
| | 7.36 (1H, td, J = 1.7, 0.7 Hz), 7.41-7.46 (2H, m), 7.47-7.55 (2H, m), 7.65 (1H, dt, J = 6.9, |
| | 2.1 Hz), 7.81 (1H, td, J = 1.9, 0.6 Hz), 8.54 (1H, t, J = 6.0 Hz) |
| 1287 | 1.84 (4H, dt, J = 10.0, 6.6 Hz), 2.34-2.41 (5H, m), 3.21 (2H, t, J = 6.6 Hz), 3.59 (2H, t, J = |
| | 5.4 Hz), 4.08 (2H, t, J = 6.6 Hz), 4.47 (2H, d, J = 6.0 Hz), 7.15 (1H, ddd, J = 7.9, 2.0, 1.0 Hz), |
| | 7.19 (1H, dt, J = 4.9, 0.8 Hz), 7.25 (1H, ddd, J = 8.0, 2.1, 1.0 Hz), 7.29 (1H, t, J = 2.0 Hz), |
| | 7.39 (1H, t, J = 8.0 Hz), 7.47-7.55 (2H, m), 7.63 (1H, dt, J = 7.2, 1.9 Hz), 7.80 (1H, dt, J = |
| | 2.4, 1.2 Hz), 8.32 (1H, d, J = 4.9 Hz), 8.44 (1H, s), 9.03 (1H, t, J = 6.0 Hz) |
| 1288 | 2.19 (3H, s), 3.21 (2H, t, J = 6.6 Hz), 3.55-3.62 (6H, m), 3.64 (3H, s), 4.04 (2H, t, J = 6.6 |
| | Hz), 4.31 (2H, d, J = 6.0 Hz), 4.37 (2H, t, J = 5.0 Hz), 4.81-4.85 (1H, m), 5.93 (1H, s), 7.04 |
| | (1H, d, J = 8.6 Hz), 7.44 (1H, dd, J = 8.6, 2.7 Hz), 7.47-7.57 (2H, m), 7.57-7.69 (2H, m), |
| | 7.80 (1H, d, J = 1.1 Hz), 8.68 (1H, t, J = 6.1 Hz) |
| 1289 | 0.21-0.28 (2H, m), 0.38-0.45 (2H, m), 0.99-1.11 (1H, m), 3.12 (2H, t, J = 6.4 Hz), 3.21 |
| | (2H, t, J = 6.6 Hz), 3.54-3.63 (6H, m), 4.04 (2H, t, J = 6.6 Hz), 4.33-4.39 (2H, m), 4.81- |
| | 4.86 (1H, m), 7.04 (1H, d, J = 8.6 Hz), 7.44 (1H, dd, J = 8.7, 2.8 Hz), 7.48-7.57 (2H, m), |
| | 7.60-7.68 (2H, m), 7.77-7.81 (1H, m), 8.51 (1H, t, J = 6.0 Hz) |
| 1290 | 2.19 (3H, s), 3.04 (2H, t, J = 6.5 Hz), 3.22 (2H, t, J = 6.6 Hz), 3.64 (3H, s), 3.71 (2H, t, J = 6.4 |
| | Hz), 4.06 (2H, t, J = 6.6 Hz), 4.31 (2H, d, J = 6.0 Hz), 4.67-4.79 (4H, m), 5.43-5.53 (1H, m), |
| | 5.94 (1H, s), 7.36 (1H, d, J = 8.2 Hz), 7.47-7.54 (3H, m), 7.61-7.68 (1H, m), 7.78-7.82 |
| | (1H, m), 7.83 (1H, d, J = 2.3 Hz), 8.68 (1H, t, J = 6.1 Hz). |
| 1292 | 0.20-0.27 (m, 2H), 0.39-0.45 (m, 2H), 0.99-1.11 (m, 1H), 1.49-1.61 (m, 1H), 1.73- |
| | 1.97 (m, 3H), 2.94 (t, J = 6.6 Hz, 2H), 3.12 (t, J = 6.4 Hz, 2H), 3.22 (t, J = 6.6 Hz, 2H), 3.42- |
| | 3.50 (m, 1H), 3.56-3.69 (m, 4H), 3.74-3.81 (m, 1H), 4.01-4.10 (m, 3H), 7.32 (d, J = 8.2 |
| | Hz, 1H), 7.46 (dd, J = 8.1, 2.4 Hz, 1H), 7.48-7.55 (m, 2H), 7.60-7.68 (m, 1H), 7.77-7.81 |
| | (m, 1H), 7.83 (d, J = 2.4 Hz, 1H), 8.51 (t, J = 6.0 Hz, 1H). |
| 1293 | 1.49-1.60 (1H, m), 1.73-1.96 (3H, m), 2.19 (3H, s), 2.94 (2H, t, J = 6.6 Hz), 3.22 (2H, t, |
| | J = 6.6 Hz), 3.43-3.50 (1H, m), 3.56-3.70 (7H, m), 3.72-3.82 (1H, m), 4.01-4.10 (3H, |
| | m), 4.31 (2H, d, J = 6.0 Hz), 5.94 (1H, s), 7.32 (1H, d, J = 8.2 Hz), 7.46 (1H, dd, J = 8.1, 2.4 Hz), |
| | 7.47-7.54 (2H, m), 7.59-7.70 (1H, m), 7.78-7.82 (1H, m), 7.83 (1H, d, J = 2.3 Hz), 8.68 |
| | (1H, t, J = 6.1 Hz). |
| 1294 | 0.16-0.28 (2H, m), 0.34-0.46 (2H, m), 0.97-1.10 (1H, m), 1.91-2.02 (2H, m), 2.71 (2H, |
| | t, J = 7.0 Hz), 3.12 (2H, t, J = 6.4 Hz), 3.16-3.26 (4H, m), 3.50-3.62 (4H, m), 4.07 (2H, t, |
| | J = 6.6 Hz), 4.74-4.83 (1H, m), 7.26 (1H, d, J = 8.2 Hz), 7.39 (1H, dd, J = 8.1, 2.3 Hz), 7.46- |
| | 7.55 (3H, m), 7.61-7.66 (1H, m), 7.77-7.81 (1H, m), 8.51 (1H, t, J = 6.0 Hz) |
| 1295 | 0.18-0.29 (2H, m), 0.37-0.49 (2H, m), 1.04 (1H, ddd, J = 12.6, 7.8, 5.0 Hz), 2.98-3.20 |
| | (8H, m), 3.27 (2H, t, J = 6.7 Hz), 4.34 (2H, t, J = 6.8 Hz), 6.97 (1H, d, J = 4.4 Hz), 7.37 (1H, d, |
| | J = 4.3 Hz), 7.53-7.62 (2H, m), 7.67 (1H, dt, J = 6.4, 2.3 Hz), 7.83 (1H, d, J = 2.2 Hz), 8.58 |
| | (1H, t, J = 6.0 Hz) |
| 1296 | 1.38 (6H, d, J = 6.7 Hz), 2.97 (2H, t, J = 6.6 Hz), 3.03 (3H, s), 3.22 (2H, t, J = 6.6 Hz), 3.55 |
| | (2H, t, J = 6.7 Hz), 4.07 (2H, t, J = 6.6 Hz), 4.40 (2H, dd, J = 6.4, 2.9 Hz), 4.42-4.47 (1H, m), |
| | 6.14 (1H, d, J = 2.2 Hz), 7.32 (1H, d, J = 8.1 Hz), 7.43-7.48 (1H, m), 7.49-7.52 (2H, m), |
| | 7.63-7.67 (2H, m), 7.81 (1H, dt, J = 2.1, 1.0 Hz), 7.83 (1H, d, J = 2.3 Hz), 8.80 (1H, t, J = 6.1 |
| | Hz) |
| 1297 | 1.12 (3H, t, J = 7.2 Hz), 2.98 (2H, t, J = 6.6 Hz), 3.04 (3H, s), 3.22 (2H, t, J = 6.6 Hz), 3.26- |
| | 3.31 (2H, m), 3.55 (2H, t, J = 6.6 Hz), 4.07 (2H, t, J = 6.6 Hz), 7.33 (1H, d, J = 8.2 Hz), 7.46 |
| | (1H, dd, J = 8.0, 2.4 Hz), 7.50-7.54 (2H, m), 7.62-7.66 (1H, m), 7.80 (1H, td, J = 1.8, 0.7 |
| | Hz), 7.83 (1H, d, J = 2.3 Hz), 8.48 (1H, t, J = 5.9 Hz) |
| 1298 | 0.19-0.28 (2H, m), 0.36-0.47 (2H, m), 0.98-1.11 (1H, m), 2.89 (3H, s), 2.98 (3H, s), 3.13 |
| | (2H, t, J = 6.5 Hz), 3.25 (2H, t, J = 6.5 Hz), 4.51 (2H, t, J = 6.4 Hz), 7.50-7.59 (2H, m), 7.68 |
| | (1H, dt, J = 6.8, 2.1 Hz), 7.86 (1H, dt, J = 2.3, 1.2 Hz), 7.96 (1H, d, J = 1.8 Hz), 8.57 (1H, t, J = |
| | 6.1 Hz), 9.10 (1H, d, J = 1.8 Hz) |
| 1299 | 2.36 (3H, s), 3.21 (2H, t, J = 6.5 Hz), 3.56-3.61 (6H, m), 4.04 (2H, t, J = 6.5 Hz), 4.37 (2H, t, |
| | J = 5.0 Hz), 4.48 (2H, d, J = 5.9 Hz), 4.81-4.85 (1H, m), 7.03 (1H, d, J = 8.6 Hz), 7.15-7.22 |
| | (1H, m), 7.44 (1H, dd, J = 8.7, 2.9 Hz), 7.48-7.55 (2H, m), 7.60-7.68 (2H, m), 7.77-7.82 |
| | (1H, m), 8.29-8.37 (1H, m), 8.44 (1H, s), 9.00 (1H, t, J = 6.0 Hz) |
| 1300 | 0.21-0.26 (2H, m), 0.39-0.44 (2H, m), 1.01-1.09 (1H, m), 2.67 (2H, t, J = 5.9 Hz), 3.12 |
| | (2H, t, J = 6.5 Hz), 3.14-3.25 (4H, m), 3.54-3.65 (2H, m), 3.73 (2H, s), 4.09 (2H, t, J = 6.6 |
| | Hz), 4.54-4.65 (1H, m), 7.28 (1H, s), 7.39 (1H, dd, J = 8.4, 2.1 Hz), 7.49-7.55 (2H, m), |
| | 7.61-7.65 (2H, m), 7.77-7.80 (1H, m), 8.52 (1H, t, J = 6.0 Hz). |
| 1301 | 0.20-0.28 (2H, m), 0.36-0.47 (2H, m), 0.99-1.10 (1H, m), 3.05 (3H, s), 3.09-3.16 (4H, |
| | m), 3.24 (2H, t, J = 6.6 Hz), 3.65 (2H, t, J = 6.8 Hz), 4.11 (2H, t, J = 6.5 Hz), 7.47-7.57 (2H, |
| | m), 7.61-7.71 (1H, m), 7.79-7.84 (1H, m), 8.18 (1H, d, J = 2.5 Hz), 8.53 (1H, t, J = 6.0 Hz), |
| | 8.64 (1H, d, J = 2.6 Hz) |
| 1302 | 2.36 (3H, s), 3.05 (3H, s), 3.12 (2H, t, J = 6.8 Hz), 3.24 (2H, t, J = 6.5 Hz), 3.64 (2H, t, J = 6.8 |
| | Hz), 4.11 (2H, t, J = 6.5 Hz), 4.48 (2H, d, J = 6.0 Hz), 7.19 (1H, d, J = 5.0 Hz), 7.46-7.56 (2H, |
| | m), 7.61-7.70 (1H, m), 7.79-7.85 (1H, m), 8.17 (1H, d, J = 2.5 Hz), 8.32 (1H, d, J = 4.9 Hz), |
| | 8.44 (1H, s), 8.63 (1H, d, J = 2.5 Hz), 9.02 (1H, t, J = 6.0 Hz) |
| 1303 | 2.17-2.21 (3H, m), 3.05 (3H, s), 3.12 (2H, t, J = 6.8 Hz), 3.24 (2H, t, J = 6.6 Hz), 3.62-3.67 |
| | (5H, m), 4.11 (2H, t, J = 6.6 Hz), 4.31 (2H, d, J = 6.1 Hz), 5.92-5.96 (1H, m), 7.46-7.56 |
| | (2H, m), 7.61-7.71 (1H, m), 7.79-7.85 (1H, m), 8.18 (1H, d, J = 2.5 Hz), 8.64 (1H, d, J = |
| | 2.6 Hz), 8.70 (1H, t, J = 6.1 Hz) |
| 1304 | 2.15 (3H, s), 2.19 (3H, s), 3.13 (2H, t, J = 8.5 Hz), 3.20 (2H, t, J = 6.6 Hz), 3.64 (3H, s), 4.00 |
| | (2H, t, J = 6.6 Hz), 4.12 (2H, t, J = 8.5 Hz), 4.31 (2H, d, J = 6.0 Hz), 5.94 (1H, s), 6.96 (1H, dd, |
| | J = 7.9, 2.1 Hz), 7.23 (1H, d, J = 8.0 Hz), 7.45-7.54 (2H, m), 7.59-7.65 (1H, m), 7.77-7.81 |
| | (1H, m), 8.00 (1H, d, J = 2.0 Hz), 8.66 (1H, t, J = 6.1 Hz) |
| 1305 | 1.82-2.04 (2H, m), 2.19 (3H, d, J = 0.7 Hz), 2.71 (2H, t, J = 7.0 Hz), 3.14-3.26 (4H, m), |
| | 3.48-3.61 (4H, m), 3.64 (3H, s), 4.07 (2H, t, J = 6.6 Hz), 4.31 (2H, d, J = 6.0 Hz), 4.80 (1H, s), |
| | 5.93 (1H, d, J = 0.8 Hz), 7.26 (1H, d, J = 8.2 Hz), 7.39 (1H, dd, J = 8.1, 2.3 Hz), 7.47-7.54 |
| | (3H, m), 7.62-7.66 (1H, m), 7.77-7.82 (1H, m), 8.69 (1H, t, J = 6.1 Hz) |
| 1306 | 1.91-2.02 (2H, m), 2.36 (3H, s), 2.70 (2H, t, J = 7.0 Hz), 3.11-3.25 (4H, m), 3.48-3.62 |
| | (4H, m), 4.07 (2H, t, J = 6.6 Hz), 4.48 (2H, d, J = 6.0 Hz), 4.80 (1H, s), 7.16-7.21 (1H, m), |
| | 7.26 (1H, d, J = 8.2 Hz), 7.39 (1H, dd, J = 8.1, 2.3 Hz), 7.46-7.55 (3H, m), 7.62-7.66 (1H, |
| | m), 7.75-7.82 (1H, m), 8.32 (1H, d, J = 4.9 Hz), 8.43 (1H, s), 9.01 (1H, t, J = 6.0 Hz). |
| 1307 | 2.94-2.99 (2H, m), 3.01 (3H, s), 3.14-3.21 (2H, m), 3.50-3.56 (2H, m), 3.65-3.79 (2H, |
| | m), 4.04 (2H, t, J = 6.6 Hz), 4.99 (1H, t, J = 5.6 Hz), 5.02-5.09 (1H, m), 7.20-7.26 (1H, m), |
| | 7.31 (3H, td, J = 8.0, 1.1 Hz), 7.37-7.41 (2H, m), 7.43 (1H, dd, J = 8.1, 2.4 Hz), 7.49-7.55 |
| | (2H, m), 7.63-7.68 (1H, m), 7.79-7.84 (2H, m), 8.61 (1H, d, J = 8.3 Hz) |
| 1308 | 2.34 (3H, s), 2.98 (2H, t, J = 6.6 Hz), 3.03 (3H, s), 3.25 (2H, t, J = 6.6 Hz), 3.55 (2H, t, J = 6.6 |
| | Hz), 4.08 (2H, t, J = 6.6 Hz), 4.60 (2H, d, J = 5.1 Hz), 7.24 (1H, dd, J = 7.5, 4.8 Hz), 7.32 (1H, |
| | d, J = 8.2 Hz), 7.46 (1H, dd, J = 8.1, 2.4 Hz), 7.50-7.57 (2H, m), 7.60-7.64 (1H, m), 7.64- |
| | 7.70 (1H, m), 7.81-7.86 (2H, m), 8.38 (1H, ddd, J = 4.9, 1.8, 0.8 Hz), 8.75 (1H, t, J = 5.1 Hz) |
| 1309 | 2.50 (3H, s), 2.93-2.98 (2H, m), 3.00 (3H, s), 3.14-3.24 (2H, m), 3.50-3.55 (2H, m), 4.04 |
| | (2H, t, J = 6.6 Hz), 4.43 (2H, d, J = 6.0 Hz), 7.17 (1H, ddd, J = 7.7, 4.8, 0.6 Hz), 7.29 (1H, |
| | d, J = 8.2 Hz), 7.43 (1H, dd, J = 8.1, 2.3 Hz), 7.47-7.52 (2H, m), 7.58 (1H, dd, J = 7.8, 1.7 Hz), |
| | 7.61-7.65 (1H, m), 7.78-7.82 (2H, m), 8.30 (1H, dd, J = 4.9, 1.7 Hz), 9.03 (1H, t, J = 6.1 |
| | Hz) |
| 1310 | 2.36 (3H, d, J = 0.9 Hz), 2.97 (2H, t, J = 6.6 Hz), 3.02 (3H, s), 3.22 (2H, t, J = 6.6 Hz), 3.54 |
| | (2H, t, J = 6.7 Hz), 4.07 (2H, t, J = 6.6 Hz), 4.77 (2H, d, J = 5.4 Hz), 7.32 (1H, s), 7.45 (1H, dd, |
| | J = 8.1, 2.3 Hz), 7.47-7.54 (3H, m), 7.63-7.67 (1H, m), 7.80-7.84 (2H, m), 8.95 (1H, t, J = |
| | 5.5 Hz), 9.00 (1H, d, J = 5.0 Hz) |
| 1311 | 0.20-0.28 (2H, m), 0.38-0.46 (2H, m), 0.98-1.10 (1H, m), 2.95 (3H, d, J = 8.4 Hz), 3.12 |
| | (2H, t, J = 6.4 Hz), 3.16 (1H, s), 3.22 (2H, t, J = 6.6 Hz), 3.29 (1H, s), 3.40 (2H, dd, J = 16.2, |
| | 8.1 Hz), 3.57 (2H, d, J = 16.0 Hz), 4.09 (2H, t, J = 6.6 Hz), 7.21-7.30 (1H, m), 7.40 (1H, dt, |
| | J = 2.6, 1.1 Hz), 7.42-7.48 (2H, m), 7.49-7.55 (2H, m), 7.64 (1H, dt, J = 6.7, 2.1 Hz), 7.80 |
| | (1H, td, J = 1.9, 0.7 Hz), 8.53 (1H, t, J = 6.1 Hz) one H hidden under water peak |
| 1312 | 0.21-0.26 (2H, m), 0.38-0.44 (2H, m), 0.98-1.10 (1H, m), 2.96 (3H, d, J = 8.5 Hz), 3.12 |
| | (2H, t, J = 6.5 Hz), 3.17-3.24 (2H, m), 3.44-3.53 (2H, m), 3.61 (1H, d, J = 6.6 Hz), 4.09 |
| | (2H, t, J = 6.5 Hz), 4.80 (1H, d, J = 5.5 Hz), 7.25-7.31 (1H, m), 7.38-7.46 (3H, m), 7.49- |
| | 7.55 (2H, m), 7.64 (1H, dt, J = 6.7, 2.1 Hz), 7.80 (1H, dt, J = 2.1, 1.1 Hz), 8.53 (1H, t, J = 6.0 |
| | Hz) |
| 1313 | 0.19-0.30 (2H, m), 0.35-0.48 (2H, m), 0.98-1.11 (1H, m), 3.12 (2H, t, J = 6.4 Hz), 3.22 |
| | (2H, t, J = 6.5 Hz), 3.73 (2H, dd, J = 6.0, 4.1 Hz), 3.96 (2H, dd, J = 6.0, 4.0 Hz), 4.09 (2H, |
| | t, J = 6.6 Hz), 4.20 (2H, s), 7.25-7.32 (2H, m), 7.40-7.46 (2H, m), 7.48-7.56 (2H, m), 7.63 |
| | (1H, dt, J = 7.0, 2.0 Hz), 7.79 (1H, t, J = 2.0 Hz), 8.53 (1H, t, J = 6.0 Hz) |
| 1314 | 1.81-1.91 (2H, m), 2.16 (3H, s), 2.36 (3H, s), 2.70 (2H, t, J = 6.6 Hz), 3.21 (2H, t, J = 6.6 |
| | Hz), 3.67 (2H, t, J = 6.3 Hz), 4.04 (2H, t, J = 6.7 Hz), 4.48 (2H, d, J = 5.9 Hz), 7.08 (1H, d, J = |
| | 8.2 Hz), 7.19 (2H, d, J = 5.2 Hz), 7.44-7.55 (3H, m), 7.60-7.67 (1H, m), 7.77-7.83 (1H, |
| | m), 8.32 (1H, d, J = 4.9 Hz), 8.43 (1H, s), 8.99 (1H, t, J = 6.0 Hz) |
| 1316 | 0.21-0.27 (2H, m), 0.35-0.49 (2H, m), 0.98-1.14 (1H, m), 3.12 (2H, t, J = 6.5 Hz), 3.21 |
| | (2H, t, J = 6.6 Hz), 3.38-3.48 (2H, m), 4.08 (2H, t, J = 6.6 Hz), 4.22-4.31 (2H, m), 4.35 (2H, |
| | s), 7.16 (1H, dt, J = 7.6, 1.4 Hz), 7.26-7.34 (2H, m), 7.40 (1H, t, J = 7.7 Hz), 7.48-7.57 (2H, |
| | m), 7.63 (1H, dt, J = 6.8, 2.1 Hz), 7.79 (1H, dt, J = 2.3, 1.2 Hz), 8.52 (1H, t, J = 6.0 Hz) |
| 1317 | 1.13 (3H, t, J = 7.1 Hz), 2.17-2.25 (3H, m), 3.21 (2H, t, J = 6.6 Hz), 3.45-3.61 (4H, m), 3.64 |
| | (3H, s), 4.04 (2H, t, J = 6.6 Hz), 4.26-4.37 (4H, m), 5.91-5.96 (1H, m), 7.04 (1H, d, J = 8.6 |
| | Hz), 7.41-7.48 (1H, m), 7.46-7.56 (2H, m), 7.59-7.68 (2H, m), 7.77-7.83 (1H, m), 8.68 |
| | (1H, t, J = 6.1 Hz) |
| 1318 | 1.13 (3H, t, J = 7.1 Hz), 2.36 (3H, s), 3.21 (2H, t, J = 6.6 Hz), 3.49-3.59 (4H, m), 4.04 (2H, t, |
| | J = 6.6 Hz), 4.30-4.37 (2H, m), 4.48 (2H, d, J = 6.0 Hz), 7.04 (1H, d, J = 8.6 Hz), 7.19 (1H, d, |
| | J = 4.9 Hz), 7.42-7.46 (1H, m), 7.47-7.56 (2H, m), 7.59-7.69 (2H, m), 7.77-7.82 (1H, |
| | m), 8.32 (1H, d, J = 4.9 Hz), 8.43 (1H, s), 9.00 (1H, t, J = 6.0 Hz) |
| 1319 | 2.15-2.21 (3H, m), 3.09 (3H, s), 3.21 (2H, t, J = 6.6 Hz), 3.55 (2H, t, J = 5.1 Hz), 3.64 (3H, |
| | s), 4.04 (2H, t, J = 6.6 Hz), 4.31 (2H, d, J = 6.0 Hz), 4.34-4.39 (2H, m), 5.85-6.01 (1H, m), |
| | 7.04 (1H, d, J = 8.6 Hz), 7.41-7.48 (1H, m), 7.49-7.55 (2H, m), 7.56-7.69 (2H, m), 7.78- |
| | 7.83 (1H, m), 8.68 (1H, t, J = 6.1 Hz) |
| 1320 | 1.15 (6H, d, J = 6.8 Hz), 2.19 (3H, d, J = 0.7 Hz), 3.21 (2H, t, J = 6.6 Hz), 3.44 (2H, t, J = 5.3 |
| | Hz), 3.64 (3H, s), 4.05 (2H, t, J = 6.6 Hz), 4.27 (2H, t, J = 5.2 Hz), 4.31 (2H, d, J = 6.1 Hz), |
| | 4.79 (1H, hept, J = 6.8 Hz), 5.93 (1H, d, J = 0.8 Hz), 7.05 (1H, d, J = 8.6 Hz), 7.45 (1H, dd, J = |
| | 8.6, 2.7 Hz), 7.48-7.55 (2H, m), 7.61 (1H, d, J = 2.8 Hz), 7.62-7.66 (1H, m), 7.79-7.81 |
| | (1H, m), 8.68 (1H, t, J = 6.1 Hz) |
| 1321 | 2.13-2.20 (3H, m), 2.99 (2H, t, J = 6.5 Hz), 3.23 (2H, t, J = 6.6 Hz), 3.59 (2H, t, J = 6.6 Hz), |
| | 4.03-4.10 (4H, m), 4.32-4.39 (2H, m), 5.91 (1H, d, J = 0.8 Hz), 7.07 (1H, s br), 7.33 (1H, d, |
| | J = 8.2 Hz), 7.44 (1H, s br), 7.46-7.55 (3H, m), 7.61-7.68 (1H, m), 7.78-7.86 (2H, m), |
| | 8.68 (1H, s br), 12.21 (1H, s br) |
| 1322 | 1.33 (3H, t, J = 7.1 Hz), 2.23 (3H, s), 3.01 (2H, s), 3.20 (2H, t, J = 6.6 Hz), 3.65 (2H, s), 4.09 |
| | (2H, t, J = 6.6 Hz), 4.35 (2H, q, J = 7.1 Hz), 7.16-7.28 (2H, m), 7.29-7.38 (2H, m), 7.47- |
| | 7.65 (3H, m), 7.73-7.78 (1H, m) |
| 1323 | 1.33 (3H, t, J = 7.1 Hz), 2.00-2.11 (2H, m), 3.21 (2H, t, J = 6.6 Hz), 3.82 (2H, t, J = 7.0 Hz), |
| | 4.09 (2H, t, J = 6.6 Hz), 4.36 (2H, q, J = 7.1 Hz), 7.09-7.16 (1H, m), 7.39 (1H, t, J = 8.1 Hz), |
| | 7.47-7.63 (4H, m), 7.68-7.78 (2H, m) |
| 1324 | 1.33 (3H, t, J = 7.1 Hz), 2.77 (3H, s), 2.95 (3H, s), 3.20 (2H, t, J = 6.6 Hz), 3.80 (3H, s), 4.00- |
| | 4.09 (2H, m), 4.35 (2H, q, J = 7.1 Hz), 7.10 (1H, d, J = 9.0 Hz), 7.19 (1H, d, J = 2.6 Hz), 7.38 |
| | (1H, dd, J = 8.9, 2.7 Hz), 7.48-7.63 (3H, m), 7.72-7.78 (1H, m) |
| 1325 | 2.98 (2H, t, J = 6.7 Hz), 3.03 (3H, s), 3.21 (2H, dd, J = 6.4, 3.2 Hz), 3.55 (2H, t, J = 6.6 Hz), |
| | 3.70 (3H, s), 4.05-4.08 (2H, m), 5.83 (1H, d, J = 7.6 Hz), 7.32 (1H, d, J = 8.2 Hz), 7.40-7.43 |
| | (1H, m), 7.45 (1H, dt, J = 8.0, 2.0 Hz), 7.50-7.55 (2H, m), 7.66 (1H, dt, J = 6.9, 2.1 Hz), 7.83 |
| | (2H, ddt, J = 2.8, 2.0, 1.2 Hz), 7.89-7.93 (1H, m), 8.54 (1H, dd, J = 4.8, 1.6 Hz), 8.68 (1H, |
| | dd, J = 2.4, 0.9 Hz), 9.32 (1H, d, J = 7.6 Hz) |
| 1326 | 2.38 (3H, s), 2.55 (3H, s), 2.94 (2H, t, J = 6.6 Hz), 3.21 (2H, t, J = 6.6 Hz), 3.49-3.63 (4H, |
| | m), 3.66 (1H, t, J = 5.7 Hz), 4.05 (2H, t, J = 6.6 Hz), 4.50 (2H, d, J = 5.4 Hz), 4.74-4.79 (1H, |
| | m), 7.03 (1H, d, J = 5.0 Hz), 7.31 (1H, d, J = 8.2 Hz), 7.43 (1H, dd, J = 8.1, 2.4 Hz), 7.46- |
| | 7.52 (3H, m), 7.61 (1H, dt, J = 6.8, 2.1 Hz), 7.76 (1H, q, J = 1.4 Hz), 7.81 (1H, dd, J = 4.0, 2.2 |
| | Hz), 8.19 (1H, d, J = 5.0 Hz), 8.67 (1H, t, J = 5.4 Hz) |
| 1327 | 1.52 (3H, d, J = 7.0 Hz), 2.40 (3H, s), 2.95 (2H, t, J = 6.5 Hz), 3.17 (2H, t, J = 6.7 Hz), 3.51- |
| | 3.64 (5H, m), 4.04 (2H, t, J = 6.7 Hz), 4.54 (1H, s), 4.77 (1H, t, J = 5.2 Hz), 5.36 (1H, q, J = |
| | 7.3 Hz), 7.16 (1H, d, J = 5.0 Hz), 7.31 (1H, d, J = 8.3 Hz), 7.44 (1H, dd, J = 8.1, 2.3 Hz), 7.48- |
| | 7.56 (2H, m), 7.66 (1H, dt, J = 6.7, 2.1 Hz), 7.82 (2H, td, J = 3.2, 2.1 Hz), 8.30 (1H, d, J = 4.9 |
| | Hz), 8.64 (1H, s), 8.98 (1H, d, J = 8.1 Hz) |
| 1328 | 4.12 (3H, td, J = 6.7, 2.7 Hz), 4.17 (4H, d, J = 2.1 Hz), 4.28-4.37 (2H, m), 4.69 (2H, td, J = |
| | 6.6, 2.4 Hz), 5.19 (2H, q, J = 7.0 Hz), , 8.45 (2H, dq, J = 8.6, 4.7 Hz), 8.56-8.60 (1H, m), |
| | 8.63-8.71 (2H, m), 8.80 (1H, dt, J = 6.4, 2.2 Hz), 8.84 (1H, dd, J = 6.0, 4.0 Hz), 8.93-9.00 (2H, |
| | m), 9.53 (1H, d, J = 4.3 Hz), 9.71 (1H, s), 9.87 (1H, d, J = 5.7 Hz) |
| 1329 | 1.15 (3H, d, J = 6.9 Hz), 2.16-2.21 (3H, m), 3.21 (2H, t, J = 6.6 Hz), 3.46-3.63 (4H, m), |
| | 3.64 (3H, s), 3.90-4.00 (1H, m), 4.03 (2H, t, J = 6.6 Hz), 4.14-4.22 (1H, m), 4.31 (2H, d, J = |
| | 6.0 Hz), 4.45 (1H, dd, J = 12.2, 6.2 Hz), 4.81 (1H, t, J = 4.7 Hz), 5.91-5.96 (1H, m), 7.03 |
| | (1H, d, J = 8.7 Hz), 7.42 (1H, dd, J = 8.7, 2.7 Hz), 7.46-7.56 (2H, m), 7.58-7.68 (1H, m), |
| | 7.77-7.83 (1H, m), 7.96 (1H, d, J = 2.7 Hz), 8.69 (1H, t, J = 6.1 Hz) |
| 1330 | 0.20-0.27 (2H, m), 0.39-0.45 (2H, m), 1.00-1.10 (1H, m), 2.99 (2H, t, J = 6.6 Hz), 3.12 |
| | (2H, t, J = 6.5 Hz), 3.22 (2H, t, J = 6.6 Hz), 3.62 (2H, t, J = 6.5 Hz), 4.07 (2H, t, J = 6.6 Hz), 4.19 |
| | (2H, s), 7.34 (1H, d, J = 8.2 Hz), 7.45-7.57 (3H, m), 7.61-7.68 (1H, m), 7.80 (1H, t, J = 1.5 |
| | Hz), 7.83 (1H, d, J = 2.3 Hz), 8.52 (1H, t, J = 6.0 Hz), 12.88 (1H, s, br) |
| 1331 | 2.19 (3H, d, J = 0.6 Hz), 2.99 (2H, t, J = 6.5 Hz), 3.22 (2H, t, J = 6.6 Hz), 3.61 (2H, t, J = 6.6 |
| | Hz), 3.64 (3H, s), 4.07 (2H, t, J = 6.6 Hz), 4.19 (2H, s), 4.31 (2H, d, J = 6.0 Hz), 5.94 (1H, d, |
| | J = 0.8 Hz), 7.34 (1H, d, J = 8.2 Hz), 7.46-7.58 (3H, m), 7.61-7.69 (1H, m), 7.76-7.82 (1H, |
| | m), 7.82-7.87 (1H, m), 8.69 (1H, t, J = 6.1 Hz), 12.83 (1H, s br) |
| 1332 | 2.16 (3H, s), 2.99 (2H, t, J = 6.6 Hz), 3.22 (2H, t, J = 6.6 Hz), 3.62 (2H, t, J = 6.6 Hz), 4.08 (2H, t, |
| | J = 6.6 Hz), 4.18 (2H, s), 4.36 (2H, d, J = 6.1 Hz), 5.91 (1H, s), 7.34 (1H, d, J = 8.2 Hz), 7.47- |
| | 7.53 (3H, m), 7.61-7.69 (1H, m), 7.78-7.82 (1H, m), 7.83 (1H, d, J = 2.3 Hz), 8.73 (1H, t, |
| | J = 6.0 Hz), 12.51 (1H, s) 1 exchangeable proton not seen |
| 1333 | 0.21-0.26 (2H, m), 0.39-0.45 (2H, m), 1.01-1.09 (1H, m), 2.99 (2H, t, J = 6.7 Hz), 3.12 |
| | (2H, t, J = 6.4 Hz), 3.22 (2H, t, J = 6.6 Hz), 3.59 (2H, t, J = 6.6 Hz), 4.04-4.12 (4H, m), 7.04- |
| | 7.11 (1H, m), 7.33 (1H, d, J = 8.2 Hz), 7.42-7.46 (1H, m), 7.47 (1H, dd, J = 8.1, 2.4 Hz), |
| | 7.49-7.53 (2H, m), 7.63-7.67 (1H, m), 7.78-7.82 (1H, m), 7.83 (1H, d, J = 2.3 Hz), 8.52 (1H, t, |
| | J = 6.0 Hz) |
| 1334 | 2.77 (3H, d, J = 4.8 Hz), 2.98 (2H, t, J = 6.6 Hz), 3.03 (3H, s), 3.21 (2H, t, J = 6.6 Hz), 3.55 |
| | (2H, t, J = 6.7 Hz), 4.06 (2H, t, J = 6.6 Hz), 7.32 (1H, d, J = 8.1 Hz), 7.45 (1H, dd, J = 8.0, 2.4 |
| | Hz), 7.46-7.56 (2H, m), 7.59-7.67 (1H, m), 7.76-7.85 (2H, m), 8.42 (1H, t, J = 4.7 Hz |
| 1335 | 2.29 (2H, p, J = 7.7 Hz), 2.98 (2H, t, J = 6.6 Hz), 3.03 (3H, s), 3.20 (2H, t, J = 6.6 Hz), 3.55 |
| | (2H, t, J = 6.7 Hz), 4.06 (4H, q, J = 6.8 Hz), 4.54 (2H, t, J = 7.7 Hz), 7.32 (1H, d, J = 8.1 Hz), |
| | 7.45 (1H, dd, J = 8.1, 2.3 Hz), 7.48-7.56 (2H, m), 7.61 (1H, ddd, J = 5.3, 3.5, 2.0 Hz), 7.76 |
| | (1H, dd, J = 2.4, 1.0 Hz), 7.83 (1H, d, J = 2.3 Hz) |
| 1336 | 1.92-2.04 (1H, m), 2.14 (1H, dtd, J = 12.5, 8.0, 6.5 Hz), 2.98 (2H, t, J = 6.6 Hz), 3.03 (3H, |
| | s), 3.21 (2H, t, J = 6.6 Hz), 3.55 (2H, t, J = 6.7 Hz), 3.59 (1H, dd, J = 8.8, 4.9 Hz), 3.71 (1H, |
| | td, J = 8.1, 6.0 Hz), 3.79-3.89 (2H, m), 4.06 (2H, t, J = 6.6 Hz), 4.48 (1H, ddt, J = 11.7, 6.8, |
| | 3.5 Hz), 7.32 (1H, d, J = 8.1 Hz), 7.45 (1H, dd, J = 8.1, 2.3 Hz), 7.48-7.55 (2H, m), 7.56- |
| | 7.71 (1H, m), 7.77-7.92 (2H, m), 8.49 (1H, d, J = 7.0 Hz) |
| 1337 | 1.14 (3H, d, J = 6.7 Hz), 2.98 (2H, t, J = 6.6 Hz), 3.03 (3H, s), 3.19-3.25 (2H, m), 3.39 (1H, |
| | dt, J = 10.6, 5.9 Hz), 3.46 (1H, dt, J = 11.0, 5.6 Hz), 3.55 (2H, t, J = 6.7 Hz), 3.99-4.09 (3H, |
| | m), 4.77 (1H, t, J = 5.7 Hz), 7.32 (1H, d, J = 8.1 Hz), 7.45 (1H, dd, J = 8.1, 2.3 Hz), 7.47-7.56 |
| | (2H, m), 7.63 (1H, dt, J = 6.9, 2.0 Hz), 7.79 (1H, td, J = 1.9, 0.6 Hz), 7.83 (1H, d, J = 2.3 Hz), |
| | 7.96 (1H, d, J = 8.4 Hz) |
| 1338 | 1.92-2.02 (1H, m), 2.07-2.19 (1H, m), 2.98 (2H, t, J = 6.7 Hz), 3.03 (3H, s), 3.21 (2H, t, J = |
| | 6.6 Hz), 3.55 (2H, t, J = 6.7 Hz), 3.59 (1H, dd, J = 8.8, 4.9 Hz), 3.71 (1H, td, J = 8.1, 6.0 Hz), |
| | 3.81-3.89 (2H, m), 4.06 (2H, t, J = 6.6 Hz), 4.43-4.53 (1H, m), 7.32 (1H, d, J = 8.2 Hz), |
| | 7.45 (1H, dd, J = 8.1, 2.4 Hz), 7.47-7.55 (2H, m), 7.65 (1H, dt, J = 6.8, 2.0 Hz), 7.80 (1H, td, |
| | J = 1.9, 0.7 Hz), 7.83 (1H, d, J = 2.3 Hz), 8.50 (1H, d, J = 7.1 Hz) |
| 1339 | 2.98 (2H, t, J = 6.6 Hz), 3.03 (3H, s), 3.14-3.19 (1H, m), 3.22 (2H, t, J = 6.7 Hz), 3.50-3.58 |
| | (4H, m), 4.06 (2H, t, J = 6.6 Hz), 4.35 (2H, t, J = 6.0 Hz), 4.62 (2H, dd, J = 7.8, 6.0 Hz), 7.31 |
| | (1H, d, J = 8.1 Hz), 7.45 (1H, dd, J = 8.1, 2.4 Hz), 7.48-7.55 (2H, m), 7.63 (1H, dt, J = 6.9, |
| | 2.0 Hz), 7.78 (1H, td, J = 2.0, 0.6 Hz), 7.83 (1H, d, J = 2.3 Hz), 8.64 (1H, t, J = 6.0 Hz) |
| 1340 | 2.98 (2H, t, J = 6.7 Hz), 3.03 (3H, s), 3.22 (2H, t, J = 6.7 Hz), 3.34 (2H, q, J = 6.2 Hz), 3.48- |
| | 3.53 (2H, m), 3.55 (2H, t, J = 6.7 Hz), 4.06 (2H, t, J = 6.6 Hz), 4.74 (1H, s), 7.32 (1H, d, J = |
| | 8.1 Hz), 7.45 (1H, dd, J = 8.1, 2.4 Hz), 7.47-7.55 (2H, m), 7.60-7.67 (1H, m), 7.79 (1H, td, |
| | J = 1.9, 0.7 Hz), 7.83 (1H, d, J = 2.3 Hz), 8.27 (1H, t, J = 5.9 Hz). |
| 1341 | 2.98 (2H, t, J = 6.7 Hz), 3.03 (3H, s), 3.22 (2H, t, J = 6.6 Hz), 3.53 (2H, s), 3.60 (2H, d, J = 4.8 |
| | Hz), 4.06 (2H, t, J = 6.6 Hz), 7.31 (1H, d, J = 8.1 Hz), 7.45 (1H, dd, J = 8.1, 2.3 Hz), 7.48- |
| | 7.56 (2H, m), 7.59-7.66 (1H, m), 7.75-7.80 (1H, m), 7.83 (1H, d, J = 2.3 Hz), 8.13 (1H, s). |
| | One exchangeable proton is not observed. |
| 1342 | 2.97 (2H, t, J = 6.6 Hz), 3.03 (3H, s), 3.20 (2H, t, J = 6.6 Hz), 3.55 (2H, t, J = 6.7 Hz), 4.06 |
| | (2H, t, J = 6.6 Hz), 4.66 (2H, t, J = 6.6 Hz), 4.72 (2H, dd, J = 7.6, 6.5 Hz), 5.04 (1H, td, J = 7.4, |
| | 6.5 Hz), 7.32 (1H, d, J = 8.1 Hz), 7.45 (1H, dd, J = 8.1, 2.4 Hz), 7.49-7.56 (2H, m), 7.63- |
| | 7.72 (1H, m), 7.78-7.85 (2H, m), 9.16 (1H, d, J = 6.8 Hz). |
| 1343 | 2.29-2.38 (2H, m), 2.98 (2H, t, J = 6.7 Hz), 3.03 (3H, s), 3.15 (2H, t, J = 6.5 Hz), 3.55 (2H, |
| | dd, J = 7.2, 6.2 Hz), 4.00 (4H, t, J = 6.7 Hz), 4.06 (2H, t, J = 6.6 Hz), 7.32 (1H, d, J = 8.1 Hz), |
| | 7.45 (1H, dd, J = 8.1, 2.3 Hz), 7.48-7.53 (2H, m), 7.58-7.65 (1H, m), 7.75-7.78 (1H, m), |
| | 7.83 (1H, d, J = 2.3 Hz). |
| 1344 | 1.15 (d, J = 6.7 Hz, 3H), 2.98 (t, J = 6.6 Hz, 2H), 3.04 (s, 3H), 3.23 (dd, J = 7.2, 6.1 Hz, 2H), |
| | 3.39 (dt, J = 10.7, 5.9 Hz, 1H), 3.47 (dt, J = 11.2, 5.6 Hz, 1H), 3.55 (t, J = 6.7 Hz, 2H), 4.06 |
| | (dt, J = 14.1, 7.3 Hz, 3H), 4.78 (t, J = 5.7 Hz, 1H), 7.32 (d, J = 8.1 Hz, 1H), 7.46 (dd, J = 8.1, |
| | 2.4 Hz, 1H), 7.48-7.56 (m, 2H), 7.64 (dt, J = 6.8, 2.0 Hz, 1H), 7.80 (td, J = 2.0, 0.6 Hz, 1H), |
| | 7.84 (d, J = 2.3 Hz, 1H), 7.96 (d, J = 8.4 Hz, 1H). |
| 1345 | 1.18 (d, J = 7.4 Hz, 3H), 2.91 (t, J = 6.6 Hz, 2H), 2.96 (s, 3H), 3.07 (d, J = 7.3 Hz, 1H), 3.15 (t, |
| | J = 6.6 Hz, 2H), 3.48 (t, J = 6.7 Hz, 2H), 3.99 (t, J = 6.6 Hz, 2H), 7.25 (d, J = 8.1 Hz, 1H), 7.39 |
| | (dd, J = 8.0, 2.4 Hz, 1H), 7.41-7.44 (m, 2H), 7.57 (d, J = 5.3 Hz, 1H), 7.72 (s, 1H), 7.77 |
| | (d, J = 2.3 Hz, 1H). |
| 1346 | 1.56-1.69 (m, 2H), 2.14 (t, J = 9.3 Hz, 4H), 2.94 (t, J = 6.6 Hz, 2H), 2.99 (s, 3H), 3.17 (t, J = |
| | 6.6 Hz, 2H), 3.51 (t, J = 6.7 Hz, 2H), 4.02 (t, J = 6.6 Hz, 2H), 4.41 (q, J = 8.3 Hz, 1H), 7.28 (d, |
| | J = 8.1 Hz, 1H), 7.42 (dd, J = 8.1, 2.3 Hz, 1H), 7.46-7.52 (m, 2H), 7.59-7.65 (m, 1H), |
| | 7.75-7.81 (m, 2H), 8.57 (d, J = 8.2 Hz, 1H). |
| 1347 | 2.98 (t, J = 6.6 Hz, 2H), 3.03 (s, 3H), 3.19 (t, J = 6.6 Hz, 2H), 3.55 (t, J = 6.7 Hz, 2H), 4.06 |
| | (t, J = 6.6 Hz, 2H), 7.32 (d, J = 8.2 Hz, 1H), 7.45 (dd, J = 8.0, 2.4 Hz, 1H), 7.49-7.53 (m, 2H), |
| | 7.63 (ddd, J = 5.3, 3.5, 2.0 Hz, 1H), 7.79 (dt, J = 2.1, 0.9 Hz, 1H), 7.83 (d, J = 2.3 Hz, 1H), |
| | 9.09 (s, 1H), 11.17 (s, 1H). |
| 1350 | 1.33 (3H, t, J = 7.1 Hz), 3.22 (2H, t, J = 6.6 Hz), 4.15 (2H, t, J = 6.6 Hz), 4.36 (2H, q, J = 7.1 |
| | Hz), 6.32 (1H, td, J = 6.7, 1.4 Hz), 6.45-6.52 (1H, m), 7.27-7.34 (1H, m), 7.44-7.47 (1H, |
| | m), 7.47-7.58 (5H, m), 7.59-7.67 (2H, m), 7.73-7.79 (1H, m) |
| 1351 | 2.19 (3H, d, J = 0.6 Hz), 3.12 (3H, s), 3.21 (2H, t, J = 6.6 Hz), 3.45-3.57 (2H, m), 3.64 (3H, |
| | s), 3.70-3.78 (1H, m), 4.03 (2H, t, J = 6.6 Hz), 4.16 (1H, dd, J = 12.4, 1.5 Hz), 4.31 (2H, d, |
| | J = 6.1 Hz), 4.59 (1H, dd, J = 12.4, 6.2 Hz), 5.01-5.07 (1H, m), 5.93 (1H, s), 7.02 (1H, d, J = |
| | 8.7 Hz), 7.42 (1H, dd, J = 8.7, 2.7 Hz), 7.46-7.56 (2H, m), 7.61-7.67 (1H, m), 7.77-7.82 |
| | (1H, m), 7.98 (1H, d, J = 2.8 Hz), 8.68 (1H, t, J = 6.1 Hz) |
| 1352 | 1.13 (3H, t, J = 7.1 Hz), 2.06-2.23 (3H, m), 3.21 (2H, t, J = 6.6 Hz), 3.50-3.58 (4H, m), 4.05 |
| | (2H, t, J = 6.6 Hz), 4.31-4.44 (4H, m), 5.91 (1H, s), 7.04 (1H, d, J = 8.6 Hz), 7.45 (1H, dd, |
| | J = 8.6, 2.7 Hz), 7.47-7.56 (2H, m), 7.59-7.69 (2H, m), 7.78-7.82 (1H, m), 8.67 (1H, s), |
| | 12.21 (1H, s). |
| 1353 | 2.98 (t, J = 6.7 Hz, 2H), 3.03 (s, 3H), 3.20 (t, J = 6.6 Hz, 2H), 3.55 (t, J = 6.7 Hz, 2H), 4.00 (t, |
| | J = 9.6 Hz, 2H), 4.08 (t, J = 6.6 Hz, 2H), 4.40 (t, J = 9.5 Hz, 2H), 7.32 (d, J = 8.1 Hz, 1H), 7.46 |
| | (dd, J = 8.0, 2.4 Hz, 1H), 7.48-7.53 (m, 2H), 7.57-7.61 (m, 1H), 7.72-7.76 (m, 1H), 7.83 |
| | (d, J = 2.4 Hz, 1H). |
| 1354 | 1.57 (s, 3H), 2.98 (t, J = 6.7 Hz, 2H), 3.02-3.06 (m, 5H), 3.55 (t, J = 6.7 Hz, 2H), 4.07 (t, J = |
| | 6.6 Hz, 2H), 4.33 (d, J = 6.8 Hz, 2H), 4.56 (s, 2H), 4.59 (d, J = 6.5 Hz, 2H), 7.32 (d, J = 8.2 Hz, |
| | 1H), 7.43-7.50 (m, 3H), 7.53-7.57 (m, 1H), 7.65-7.69 (m, 1H), 7.83 (d, J = 2.3 Hz, 1H). |
| 1355 | 0.89-1.03 (4H, m), 2.96 (2H, t, J = 6.5 Hz), 3.23 (2H, t, J = 6.6 Hz), 3.53-3.69 (7H, m), 4.07 |
| | (2H, t, J = 6.6 Hz), 4.38 (2H, d, J = 6.1 Hz), 4.78 (1H, s), 6.14 (1H, d, J = 2.3 Hz), 7.33 (1H, d, |
| | J = 8.2 Hz), 7.46 (1H, dd, J = 8.1, 2.4 Hz), 7.50-7.53 (2H, m), 7.65 (2H, dd, J = 6.3, 2.2 Hz), |
| | 7.80-7.83 (1H, m), 7.84 (1H, d, J = 2.3 Hz), 8.79 (1H, t, J = 6.1 Hz) |
| 1356 | 2.32 (3H, d, J = 0.7 Hz), 2.98 (4H, dt, J = 6.8, 3.3 Hz), 3.02 (3H, s), 3.52-3.57 (2H, m), 4.02- |
| | 4.08 (2H, m), 4.62 (2H, s), 4.65 (2H, s), 7.22 (1H, dt, J = 4.9, 0.8 Hz), 7.31 (1H, d, J = 8.1 |
| | Hz), 7.42-7.48 (3H, m), 7.56 (1H, ddd, J = 6.7, 2.7, 2.0 Hz), 7.67-7.70 (1H, m), 7.82 (1H, |
| | d, J = 2.3 Hz), 8.38 (1H, d, J = 4.9 Hz), 8.45 (1H, s) |
| 1357 | 2.32 (3H, s), 2.96 (2H, t, J = 6.5 Hz), 3.23 (2H, t, J = 6.6 Hz), 3.50-3.66 (6H, m), 4.08 (2H, t, |
| | J = 6.6 Hz), 4.45 (2H, d, J = 6.1 Hz), 4.75-4.80 (1H, m), 7.21 (1H, d, J = 5.0 Hz), 7.33 (1H, d, |
| | J = 8.2 Hz), 7.46 (1H, dd, J = 8.0, 2.3 Hz), 7.50-7.56 (2H, m), 7.66-7.70 (1H, m), 7.82- |
| | 7.85 (2H, m), 8.33-8.37 (2H, m), 9.10 (1H, t, J = 6.2 Hz) |
| 1358 | 2.97 (2H, t, J = 6.7 Hz), 3.03 (3H, s), 3.04 (2H, d, J = 6.6 Hz), 3.55 (2H, t, J = 6.7 Hz), 3.91 |
| | (2H, s), 4.05 (2H, t, J = 6.6 Hz), 4.63 (2H, s), 7.31 (1H, d, J = 8.1 Hz), 7.41-7.50 (3H, m), |
| | 7.55 (1H, dt, J = 7.2, 2.1 Hz), 7.65-7.70 (1H, m), 7.83 (1H, d, J = 2.3 Hz). |
| 1359 | 0.57-0.63 (2H, m), 0.74-0.80 (2H, m), 1.37 (3H, s), 2.97 (2H, t, J = 6.7 Hz), 3.03 (3H, s), |
| | 3.21 (2H, t, J = 6.6 Hz), 3.55 (2H, t, J = 6.7 Hz), 4.05 (2H, t, J = 6.6 Hz), 7.31 (1H, d, J = 8.1 |
| | Hz), 7.42-7.47 (1H, m), 7.48-7.53 (2H, m), 7.61-7.65 (1H, m), 7.78-7.81 (1H, m), 7.83 |
| | (1H, d, J = 2.3 Hz), 8.56 (1H, s) |
| 1360 | 0.71-0.81 (4H, m), 2.98 (2H, t, J = 6.6 Hz), 3.03 (3H, s), 3.21 (2H, t, J = 6.6 Hz), 3.48-3.59 |
| | (4H, m), 4.06 (2H, t, J = 6.6 Hz), 4.70 (1H, t, J = 5.8 Hz), 7.32 (1H, d, J = 8.1 Hz), 7.42-7.54 |
| | (3H, m), 7.60-7.68 (1H, m), 7.80 (1H, dt, J = 2.0, 1.0 Hz), 7.83 (1H, d, J = 2.3 Hz), 8.45 (1H, |
| | s) |
| 1361 | 1.20 (q, J = 4.2 Hz, 2H), 1.31 (s, 2H), 2.98 (t, J = 6.6 Hz, 2H), 3.03 (s, 3H), 3.21 (t, J = 6.6 Hz, |
| | 2H), 3.55 (t, J = 6.6 Hz, 2H), 4.07 (t, J = 6.6 Hz, 2H), 7.32 (d, J = 8.2 Hz, 1H), 7.46 (dd, J = |
| | 8.0, 2.4 Hz, 1H), 7.49-7.56 (m, 2H), 7.65 (dt, J = 6.6, 2.2 Hz, 1H), 7.80 (d, J = 2.2 Hz, 1H), |
| | 7.84 (d, J = 2.3 Hz, 1H), 8.88 (s, 1H) |
| 1362 | 1.11 (3H, t, J = 7.2 Hz), 3.21 (2H, t, J = 6.6 Hz), 3.28 (2H, dd, J = 7.3, 6.0 Hz), 4.03-4.10 |
| | (4H, m), 4.40-4.46 (2H, m), 7.10-7.14 (1H, m), 7.40-7.43 (2H, m), 7.50-7.53 (2H, m), |
| | 7.58-7.65 (2H, m), 7.77-7.80 (1H, m), 8.46 (1H, t, J = 5.9 Hz) |
| 1363 | 1.11 (3H, t, J = 7.2 Hz), 2.05-2.13 (2H, m), 3.21 (2H, t, J = 6.6 Hz), 3.25-3.30 (2H, m), 3.65 |
| | (2H, t, J = 6.1 Hz), 4.08 (2H, t, J = 6.6 Hz), 4.33 (2H, d, J = 5.3 Hz), 7.20-7.28 (2H, m), 7.36- |
| | 7.41 (2H, m), 7.49-7.55 (2H, m), 7.60-7.65 (1H, m), 7.78 (1H, t, J = 1.8 Hz), 8.46 (1H, t, |
| | J = 5.9 Hz) |
| 1364 | 1.11 (3H, t, J = 7.2 Hz), 2.75 (3H, s), 3.20 (2H, t, J = 6.6 Hz), 3.25-3.29 (2H, m), 3.40-3.46 |
| | (2H, m), 3.73-3.81 (2H, m), 4.05 (2H, t, J = 6.6 Hz), 6.98 (1H, ddd, J = 7.9, 2.0, 1.0 Hz), |
| | 7.32 (1H, t, J = 8.1 Hz), 7.38-7.43 (1H, m), 7.49-7.53 (2H, m), 7.59-7.65 (2H, m), 7.76- |
| | 7.80 (1H, m), 8.45 (1H, t, J = 5.9 Hz) |
| 1365 | 1.11 (3H, t, J = 7.2 Hz), 1.99-2.11 (2H, m), 3.17-3.32 (4H, m), 3.82 (2H, t, J = 7.0 Hz), 4.06 |
| | (2H, t, J = 6.6 Hz), 7.13 (1H, ddd, J = 8.0, 2.0, 1.0 Hz), 7.39 (1H, t, J = 8.1 Hz), 7.46-7.57 |
| | (3H, m), 7.63 (1H, dt, J = 6.8, 2.1 Hz), 7.69 (1H, t, J = 2.1 Hz), 7.79 (1H, dt, J = 2.2, 1.1 Hz), |
| | 8.46 (1H, t, J = 5.9 Hz) 2H obscured by DMSO peak |
| 1366 | 1.11 (3H, t, J = 7.2 Hz), 2.39 (2H, p, J = 6.9 Hz), 3.17-3.32 (4H, m), 3.51 (2H, t, J = 7.4 Hz), |
| | 3.73 (2H, t, J = 6.5 Hz), 4.06 (2H, t, J = 6.6 Hz), 7.07-7.15 (2H, m), 7.17 (1H, t, J = 2.1 Hz), |
| | 7.39 (1H, t, J = 8.1 Hz), 7.46-7.57 (2H, m), 7.63 (1H, dt, J = 6.8, 2.0 Hz), 7.79 (1H, dt, J = |
| | 2.4, 1.3 Hz), 8.46 (1H, t, J = 5.9 Hz) |
| 1367 | 0.47 (1H, td, J = 5.3, 2.3 Hz), 1.11 (3H, t, J = 7.2 Hz), 1.55-1.62 (1H, m), 2.97 (1H, dd, J = |
| | 18.2, 7.3 Hz), 3.25 (4H, dt, J = 26.8, 6.7 Hz), 3.55 (1H, t, J = 6.0 Hz), 4.08 (2H, t, J = 6.6 Hz), |
| | 7.15 (1H, ddd, J = 8.0, 2.1, 1.0 Hz), 7.41 (1H, t, J = 8.1 Hz), 7.46-7.58 (3H, m), 7.63 (1H, dt, |
| | J = 6.8, 2.1 Hz), 7.69 (1H, t, J = 2.0 Hz), 7.79 (1H, dt, J = 2.4, 1.2 Hz), 8.46 (1H, t, J = 5.9 Hz) |
| | 2H obscured by DMSO peak |
| 1368 | 0.71 (1H, q, J = 4.0 Hz), 1.07-1.20 (4H, m), 1.99-2.09 (2H, m), 3.16-3.32 (4H, m), 3.72 |
| | (1H, d, J = 10.3 Hz), 3.97-4.08 (3H, m), 7.08 (1H, ddd, J = 7.8, 2.1, 1.1 Hz), 7.35 (1H, t, J = |
| | 8.1 Hz), 7.41 (1H, ddd, J = 8.3, 2.2, 1.1 Hz), 7.46-7.57 (2H, m), 7.58-7.66 (2H, m), 7.78 |
| | (1H, td, J = 1.9, 0.6 Hz), 8.46 (1H, t, J = 5.9 Hz) |
| 1369 | 0.63-0.68 (2H, m), 1.12 (5H, td, J = 7.0, 3.0 Hz), 2.49 (3H, s), 3.16-3.32 (4H, m), 3.85 (2H, |
| | S), 4.06 (2H, t, J = 6.6 Hz), 6.99 (1H, ddd, J = 7.9, 2.0, 1.0 Hz), 7.33 (1H, t, J = 8.1 Hz), 7.43 |
| | (1H, ddd, J = 8.4, 2.2, 1.0 Hz), 7.46-7.59 (3H, m), 7.62 (1H, dt, J = 6.9, 2.0 Hz), 7.78 (1H, |
| | dt, J = 2.3, 1.2 Hz), 8.46 (1H, t, J = 5.9 Hz) |
| 1370 | 2.17 (3H, s), 3.11 (3H, s), 3.23 (2H, t, J = 6.6 Hz), 3.30 (3H, s), 3.72 (1H, d, J = 15.0 Hz), 4.04- |
| | 4.15 (3H, m), 4.30-4.44 (2H, m), 5.91 (1H, s), 7.44 (1H, d, J = 8.9 Hz), 7.47-7.55 (2H, m), |
| | 7.60 (1H, dd, J = 8.8, 2.7 Hz), 7.63-7.68 (1H, m), 7.69 (1H, d, J = 2.6 Hz), 7.79-7.84 (1H, m), |
| | 8.61-8.97 (1H, m), 12.22 (1H, br s). |
| 1371 | 1.09-1.15 (6H, m), 3.21 (2H, t, J = 6.6 Hz), 3.24-3.31 (2H, m), 3.51-3.57 (4H, m), 4.04 |
| | (2H, t, J = 6.6 Hz), 4.33 (2H, t, J = 4.6 Hz), 7.04 (1H, d, J = 8.6 Hz), 7.44 (1H, dd, J = 8.6, 2.8 Hz), |
| | 7.46-7.57 (2H, m), 7.60-7.66 (2H, m), 7.77-7.80 (1H, m), 8.46 (1H, t, J = 5.9 Hz). |
| 1372 | 2.18-2.20 (3H, m), 3.11 (3H, s), 3.23 (2H, t, J = 6.6 Hz), 3.30 (3H, s), 3.65 (3H, s), 3.72 (1H, |
| | d, J = 15.0 Hz), 4.02-4.17 (3H, m), 4.31 (2H, d, J = 6.0 Hz), 5.92-5.95 (1H, m), 7.44 (1H, d, |
| | J = 8.8 Hz), 7.48-7.54 (2H, m), 7.60 (1H, dd, J = 8.8, 2.6 Hz), 7.63-7.68 (1H, m), 7.69 (1H, d, |
| | J = 2.6 Hz), 7.80-7.83 (1H, m), 8.70 (1H, t, J = 6.1 Hz). |
| 1373 | 1.15 (3H, d, J = 6.9 Hz), 2.16 (3H, s), 3.21 (2H, t, J = 6.6 Hz), 3.44-3.68 (4H, m), 3.90-4.00 |
| | (1H, m), 4.03 (2H, t, J = 6.6 Hz), 4.15-4.22 (1H, m), 4.36 (2H, d, J = 5.9 Hz), 4.45 (1H, dd, |
| | J = 12.2, 6.2 Hz), 4.80 (1H, t, J = 4.7 Hz), 5.91 (1H, d, J = 0.8 Hz), 7.03 (1H, d, J = 8.7 Hz), 7.42 |
| | (1H, dd, J = 8.7, 2.8 Hz), 7.46-7.56 (2H, m), 7.58-7.68 (1H, m), 7.77-7.83 (1H, m), 7.96 |
| | (1H, d, J = 2.8 Hz), 8.70 (1H, s), 12.21 (1H, s) |
| 1374 | 0.22-0.26 (2H, m), 0.39-0.44 (2H, m), 0.99-1.10 (1H, m), 1.13 (3H, t, J = 7.1 Hz), 3.12 |
| | (2H, t, J = 6.5 Hz), 3.21 (2H, t, J = 6.6 Hz), 3.51-3.57 (4H, m), 4.04 (2H, t, J = 6.6 Hz), 4.33 (2H, |
| | t, J = 5.1 Hz), 7.04 (1H, d, J = 8.6 Hz), 7.44 (1H, dd, J = 8.6, 2.8 Hz), 7.49-7.55 (2H, m), 7.61- |
| | 7.65 (2H, m), 7.78-7.81 (1H, m), 8.51 (1H, t, J = 6.0 Hz). |
| 1375 | 1.11 (3H, t, J = 7.2 Hz), 3.11 (3H, s), 3.22 (2H, t, J = 6.7 Hz), 3.26-3.30 (5H, m), 3.72 (1H, d, |
| | J = 15.0 Hz), 4.05-4.13 (3H, m), 7.44 (1H, d, J = 8.8 Hz), 7.49-7.54 (2H, m), 7.60 (1H, dd, |
| | J = 8.8, 2.6 Hz), 7.62-7.66 (1H, m), 7.68 (1H, d, J = 2.6 Hz), 7.78-7.81 (1H, m), 8.47 (1H, t, |
| | J = 5.9 Hz). |
| 1376 | 1.67-1.75 (2H, m), 2.36 (3H, s), 3.21 (2H, t, J = 6.6 Hz), 3.44 (2H, q, J = 6.1 Hz), 3.51-3.59 |
| | (4H, m), 4.04 (2H, t, J = 6.6 Hz), 4.34 (2H, t, J = 5.1 Hz), 4.48 (2H, d, J = 6.0 Hz), 4.50 (1H, t, |
| | J = 5.2 Hz), 7.04 (1H, d, J = 8.7 Hz), 7.19 (1H, d, J = 5.0 Hz), 7.44 (1H, dd, J = 8.6, 2.7 Hz), 7.48- |
| | 7.54 (2H, m), 7.61 (1H, d, J = 2.8 Hz), 7.62-7.66 (1H, m), 7.78-7.81 (1H, m), 8.32 (1H, d, |
| | J = 4.9 Hz), 8.43 (1H, s), 9.00 (1H, t, J = 6.0 Hz). |
| 1377 | 1.67-1.75 (2H, m), 2.19 (3H, d, J = 0.7 Hz), 3.21 (2H, t, J = 6.6 Hz), 3.44 (2H, q, J = 5.1 Hz), |
| | 3.52-3.59 (4H, m), 3.64 (3H, s), 4.04 (2H, t, J = 6.6 Hz), 4.31 (2H, d, J = 6.1 Hz), 4.34 (2H, t, |
| | J = 4.6 Hz), 4.50 (1H, t, J = 5.2 Hz), 5.92-5.95 (1H, m), 7.04 (1H, d, J = 8.6 Hz), 7.45 (1H, dd, |
| | J = 8.6, 2.7 Hz), 7.47-7.54 (2H, m), 7.61 (1H, d, J = 2.7 Hz), 7.62-7.66 (1H, m), 7.79-7.81 |
| | (1H, m), 8.68 (1H, t, J = 6.1 Hz). |
| 1378 | 2.16 (3H, s), 3.22 (2H, t, J = 6.6 Hz), 3.70 (2H, t, J = 5.1 Hz), 4.06 (2H, t, J = 6.6 Hz), 4.30- |
| | 4.39 (4H, m), 4.44 (2H, q, J = 9.5 Hz), 5.91 (1H, s), 7.09 (1H, d, J = 8.7 Hz), 7.47-7.55 (3H, |
| | m), 7.61-7.67 (2H, m), 7.78-7.81 (1H, m), 8.72 (1H, t, J = 6.0 Hz) 12.06 (1H, br.s). |
| 1379 | 1.15 (3H, d, J = 7.0 Hz), 2.19 (3H, d, J = 0.7 Hz), 3.21 (2H, t, J = 6.6 Hz), 3.46-3.63 (4H, m), |
| | 3.64 (3H, s), 3.92-4.00 (1H, m), 4.03 (2H, t, J = 6.7 Hz), 4.18 (1H, d, J = 10.8 Hz), 4.31 (2H, |
| | d, J = 6.0 Hz), 4.45 (1H, dd, J = 12.2, 6.2 Hz), 4.80 (1H, t, J = 4.9 Hz), 5.93 (1H, d, J = 0.9 Hz), |
| | 7.03 (1H, d, J = 8.7 Hz), 7.42 (1H, dd, J = 8.7, 2.8 Hz), 7.47-7.55 (2H, m), 7.60-7.67 (1H, |
| | m), 7.76-7.81 (1H, m), 7.96 (1H, d, J = 2.8 Hz), 8.68 (1H, t, J = 6.1 Hz) |
| 1380 | 1.15 (3H, d, J = 7.0 Hz), 2.18 (3H, d, J = 10.2 Hz), 3.21 (2H, t, J = 6.5 Hz), 3.45-3.66 (4H, |
| | m), 3.96 (1H, q, J = 6.8 Hz), 4.03 (2H, t, J = 6.6 Hz), 4.11-4.23 (1H, m), 4.29-4.40 (2H, m), |
| | 4.45 (1H, dd, J = 12.2, 6.2 Hz), 4.73-4.84 (1H, m), 5.91 (1H, s), 7.03 (1H, d, J = 8.7 Hz), |
| | 7.38-7.46 (1H, m), 7.48-7.55 (2H, m), 7.58-7.66 (1H, m), 7.77-7.82 (1H, m), 7.96 (1H, |
| | d, J = 2.8 Hz), 8.69 (1H, br.s), 12.20 (1H, br.s) |
| 1381 | 1.11 (3H, t, J = 7.2 Hz), 1.14 (3H, d, J = 7.0 Hz), 3.20 (2H, t, J = 6.6 Hz), 3.24-3.30 (2H, m), |
| | 3.47-3.55 (1H, m), 3.55-3.64 (3H, m), 3.91-3.99 (1H, m), 4.02 (2H, t, J = 6.7 Hz), |
| | 4.18(1H, d, J = 11.5 Hz), 4.45 (1H, dd, J = 12.2, 6.2 Hz), 4.76-4.83 (1H, m), 7.03 (1H, d, J = |
| | 8.7 Hz), 7.42 (1H, dd, J = 8.7, 2.8 Hz), 7.47-7.55 (2H, m), 7.60-7.65 (1H, m), 7.76-7.80 |
| | (1H, m), 7.96 (1H, d, J = 2.8 Hz), 8.45(1H, t, J = 5.9 Hz) |
| 1382 | 2.16 (3H, s), 3.22 (2H, t, J = 6.6 Hz), 3.65 (2H, t, J = 5.1 Hz), 3.93-4.02 (2H, m), 4.05 (2H, t, |
| | J = 6.7 Hz), 4.31-4.40 (4H, m), 5.91 (1H, s), 6.23 (1H, tt, J = 55.6 (H-F), 4.0 Hz), 7.07 (1H, |
| | d, J = 8.7 Hz), 7.46-7.55 (3H, m), 7.61-7.68 (2H, m), 7.77-7.83 (1H, m), 8.72 (1H, t, J = |
| | 5.9 Hz), 12.19 (1H, s br) |
| 1383 | 2.09-2.26 (6H, m), 3.21 (2H, t, J = 6.6 Hz), 3.60 (2H, t, J = 5.1 Hz), 3.62-3.66 (6H, m), 4.04 |
| | (2H, t, J = 6.6 Hz), 4.13 (2H, d, J = 5.7 Hz), 4.19 (2H, s), 4.31 (2H, d, J = 6.0 Hz), 4.42 (2H, |
| | t, J = 4.9 Hz), 5.90 (1H, s), 5.93 (1H, s), 7.05 (1H, d, J = 8.6 Hz), 7.46 (1H, dd, J = 8.7, 2.8 Hz), |
| | 7.49-7.56 (2H, m), 7.61-7.68 (2H, m), 7.78-7.82 (1H, m), 8.34 (1H, t, J = 5.7 Hz), 8.69 |
| | (1H, t, J = 6.1 Hz) |
| 1384 | 2.98 (2H, t, J = 6.6 Hz), 3.03 (3H, s), 3.22 (2H, t, J = 6.6 Hz), 3.49-3.64 (4H, m), 4.07 (2H, t, |
| | J = 6.6 Hz), 4.48 (1H, t, J = 5.2 Hz), 4.60 (1H, t, J = 5.2 Hz), 7.32 (1H, d, J = 8.1 Hz), 7.45 (1H, |
| | dd, J = 8.1, 2.3 Hz), 7.48-7.55 (2H, m), 7.61-7.68 (1H, m), 7.77-7.82 (1H, m), 7.83 (1H, |
| | d, J = 2.3 Hz), 8.60 (1H, t, J = 5.8 Hz) |
| 1385 | 1.86 (s, 3H), 2.96 (dt, J = 13.3, 6.6 Hz, 4H), 3.03 (s, 3H), 3.55 (t, J = 6.6 Hz, 2H), 4.04 (t, J = |
| | 6.6 Hz, 2H), 4.34 (d, J = 5.8 Hz, 2H), 7.31 (d, J = 8.1 Hz, 1H), 7.41-7.50 (m, 3H), 7.54 (dt, J = |
| | 7.5, 1.9 Hz, 1H), 7.66 (t, J = 1.8 Hz, 1H), 7.82 (d, J = 2.3 Hz, 1H), 8.37 (t, J = 5.8 Hz, 1H) |
| 1386 | 2.44 (2H, d, J = 7.1 Hz), 2.94 (2H, t, J = 6.6 Hz), 3.00 (3H, s), 3.18 (2H, t, J = 6.6 Hz), 3.42 |
| | (2H, q, J = 6.8 Hz), 3.51 (2H, t, J = 6.7 Hz), 4.03 (2H, t, J = 6.6 Hz), 7.28 (1H, d, J = 8.2 Hz), |
| | 7.42 (1H, dd, J = 8.1, 2.3 Hz), 7.47-7.51 (2H, m), 7.59 (1H, dt, J = 6.4, 2.2 Hz), 7.73-7.76 |
| | (1H, m), 7.80 (1H, d, J = 2.3 Hz), 8.43 (1H, t, J = 5.9 Hz) exchangeable proton not seen |
| 1387 | 1.33 (3H, d, J = 7.0 Hz), 2.98 (2H, t, J = 6.6 Hz), 3.03 (3H, s), 3.22 (2H, t, J = 6.6 Hz), 3.55 |
| | (2H, t, J = 6.6 Hz), 3.96-4.15 (3H, m), 7.31 (1H, d, J = 8.2 Hz), 7.45 (1H, dd, J = 8.1, 2.3 Hz), |
| | 7.49-7.56 (2H, m), 7.63 (1H, dt, J = 6.3, 2.3 Hz), 7.78 (1H, d, J = 2.3 Hz), 7.83 (1H, d, J = |
| | 2.3 Hz), 8.37 (1H, d, J = 6.5 Hz) exchangeable proton not seen |
| 1388 | 2.98 (2H, t, J = 6.6 Hz), 3.03 (3H, s), 3.16 (2H, t, J = 6.5 Hz), 3.55 (2H, t, J = 6.6 Hz), 4.10 |
| | (2H, t, J = 6.5 Hz), 7.33 (1H, d, J = 8.1 Hz), 7.41-7.48 (1H, m), 7.51-7.61 (2H, m), 7.61- |
| | 7.67 (1H, m), 7.77-7.86 (2H, m) |
| 1389 | 0.69 (4H, q, J = 2.1 Hz), 1.11 (3H, t, J = 7.2 Hz), 2.56 (2H, s), 3.17-3.34 (4H, m), 3.74 (2H, |
| | s), 4.07 (2H, t, J = 6.6 Hz), 7.14 (1H, ddd, J = 8.0, 2.1, 1.0 Hz), 7.39 (1H, t, J = 8.1 Hz), 7.46- |
| | 7.57 (3H, m), 7.57-7.68 (2H, m), 7.78 (1H, t, J = 2.0 Hz), 8.46 (1H, t, J = 5.9 Hz) |
| 1390 | 1.11 (3H, t, J = 7.1 Hz), 2.02 (2H, p, J = 5.9 Hz), 2.84 (3H, s), 3.20 (2H, t, J = 6.6 Hz), 3.28 |
| | (2H, dd, J = 7.3, 6.0 Hz), 3.60-3.67 (2H, m), 4.05 (2H, t, J = 6.6 Hz), 7.07-7.16 (2H, m), |
| | 7.24-7.34 (2H, m), 7.46-7.57 (2H, m), 7.62 (1H, dt, J = 6.9, 2.0 Hz), 7.78 (1H, t, J = 1.6 |
| | Hz), 8.46 (1H, t, J = 5.9 Hz) 2H overlaps with water peak |
| 1391 | 1.36 (3H, d, J = 7.1 Hz), 2.98 (2H, t, J = 6.6 Hz), 3.03 (3H, s), 3.22 (2H, t, J = 6.7 Hz), 3.55 |
| | (2H, t, J = 6.7 Hz), 4.06 (2H, td, J = 7.1, 2.6 Hz), 4.21 (1H, s), 7.32 (1H, d, J = 8.2 Hz), 7.45 |
| | (1H, dd, J = 8.0, 2.4 Hz), 7.48-7.55 (2H, m), 7.64 (1H, dt, J = 6.6, 2.2 Hz), 7.76-7.85 (2H, |
| | m), 8.42 (1H, s) |
| 1392 | 1.11 (3H, t, J = 7.1 Hz), 1.15 (3H, d, J = 6.9 Hz), 3.16-3.31 (4H, m), 3.44-3.64 (4H, m), |
| | 3.95 (1H, h, J = 6.5 Hz), 4.02 (2H, t, J = 6.6 Hz), 4.15-4.22 (1H, m), 4.40-4.49 (1H, m), |
| | 4.80 (1H, t, J = 4.9 Hz), 7.03 (1H, d, J = 8.7 Hz), 7.38-7.46 (1H, m), 7.46-7.56 (2H, m), |
| | 7.58-7.66 (1H, m), 7.76-7.81 (1H, m), 7.96 (1H, d, J = 2.7 Hz), 8.45 (1H, t, J = 5.9 Hz) |
| 1393 | 2.19 (3H, d, J = 0.7 Hz), 3.21 (2H, t, J = 6.6 Hz), 3.56-3.61 (2H, m), 3.64 (3H, s), 4.05 (2H, t, |
| | J = 6.6 Hz), 4.14 (2H, s), 4.31 (2H, d, J = 6.0 Hz), 4.41 (2H, dd, J = 5.5, 4.3 Hz), 5.93 (1H, |
| | d, J = 0.9 Hz), 7.04 (1H, d, J = 8.7 Hz), 7.09 (1H, s br), 7.43-7.50 (2H, m), 7.49-7.55 (2H, m), |
| | 7.62-7.68 (2H, m), 7.76-7.83 (1H, m), 8.68 (1H, t, J = 6.0 Hz) |
| 1394 | 2.20 (3H, s), 3.21 (2H, t, J = 6.6 Hz), 3.59 (2H, t, J = 4.8 Hz), 3.70 (3H, s), 4.05 (2H, t, J = 6.6 |
| | Hz), 4.14 (2H, s), 4.37-4.45 (4H, m), 7.04 (1H, d, J = 8.7 Hz), 7.09 (1H, s br), 7.43-7.48 |
| | (2H, m), 7.49-7.54 (2H, m), 7.61-7.68 (2H, m), 7.78-7.83 (1H, m), 8.58 (1H, t, J = 5.8 Hz) |
| 1395 | 2.19 (3H, s), 3.12 (3H, s), 3.21 (2H, t, J = 6.6 Hz), 3.45-3.56 (2H, m), 3.64 (3H, s), 3.70- |
| | 3.77 (1H, m), 4.03 (2H, t, J = 6.6 Hz), 4.16 (1H, dd, J = 12.3, 1.6 Hz), 4.31 (2H, d, J = 6.0 Hz), |
| | 4.59 (1H, dd, J = 12.4, 6.2 Hz), 5.02-5.06 (1H, m), 5.93 (1H, s), 7.02 (1H, d, J = 8.7 Hz), |
| | 7.42 (1H, dd, J = 8.7, 2.8 Hz), 7.47-7.55 (2H, m), 7.61-7.67 (1H, m), 7.78-7.82 (1H, m), |
| | 7.98 (1H, d, J = 2.8 Hz), 8.68 (1H, t, J = 6.1 Hz) |
| 1396 | 2.36 (3H, s), 3.12 (3H, s), 3.21 (2H, t, J = 6.6 Hz), 3.45-3.57 (2H, m), 3.69-3.78 (1H, m), |
| | 4.03 (2H, t, J = 6.6 Hz), 4.16 (1H, dd, J = 12.4, 1.5 Hz), 4.48 (2H, d, J = 6.0 Hz), 4.59 (1H, dd, |
| | J = 12.4, 6.2 Hz), 4.99-5.07 (1H, m), 7.02 (1H, d, J = 8.7 Hz), 7.19 (1H, d, J = 5.0 Hz), 7.41 |
| | (1H, dd, J = 8.7, 2.8 Hz), 7.46-7.57 (2H, m), 7.61-7.67 (1H, m), 7.77-7.82 (1H, m), 7.97 |
| | (1H, d, J = 2.7 Hz), 8.32 (1H, d, J = 4.9 Hz), 8.43 (1H, s), 9.00 (1H, t, J = 6.0 Hz) |
| 1397 | 1.08-1.16 (6H, m), 1.61-1.71 (1H, m), 2.23-2.33 (1H, m), 2.35-2.44 (1H, m), 2.51- |
| | 2.59 (1H, m), 3.18-3.30 (4H, m), 4.07 (2H, t, J = 6.6 Hz), 4.31-4.43 (1H, m), 7.16-7.21 |
| | (1H, m), 7.30-7.35 (1H, m), 7.40 (1H, t, J = 8.0 Hz), 7.47-7.54 (3H, m), 7.60-7.65 (1H, |
| | m), 7.77-7.80 (1H, m), 8.46 (1H, t, J = 5.9 Hz) |
| 1398 | 1.06-1.18 (6H, m), 1.59-1.71 (1H, m), 2.23-2.33 (1H, m), 2.35-2.45 (1H, m), 2.51- |
| | 2.59 (1H, m), 3.17-3.31 (4H, m), 4.07 (2H, t, J = 6.6 Hz), 4.32-4.43 (1H, m), 7.16-7.21 |
| | (1H, m), 7.30-7.35 (1H, m), 7.40 (1H, t, J = 8.0 Hz), 7.47-7.55 (3H, m), 7.61-7.65 (1H, |
| | m), 7.76-7.80 (1H, m), 8.46 (1H, t, J = 5.9 Hz) |
| 1399 | 1.12 (3H, t, J = 7.2 Hz), 3.18 (3H, s), 3.20-3.32 (4H, m), 4.10 (2H, t, J = 6.6 Hz), 6.74 (1H, d, |
| | J = 3.1 Hz), 7.03 (1H, d, J = 3.2 Hz), 7.23 (1H, ddd, J = 8.0, 2.0, 1.0 Hz), 7.45 (1H, t, J = 8.1 |
| | Hz), 7.48-7.57 (2H, m), 7.57-7.67 (2H, m), 7.73-7.81 (2H, m), 8.46 (1H, t, J = 5.9 Hz) |
| 1400 | 0.84-0.90 (2H, m), 0.93-0.99 (2H, m), 1.11 (3H, t, J = 7.2 Hz), 2.19 (2H, d, J = 7.4 Hz), |
| | 3.21 (2H, t, J = 6.6 Hz), 3.26-3.30 (2H, m), 3.87-3.94 (2H, m), 4.06 (2H, t, J = 6.6 Hz), 7.12 |
| | (1H, ddd, J = 7.9, 2.0, 1.0 Hz), 7.39 (1H, t, J = 8.1 Hz), 7.47-7.55 (3H, m), 7.62 (1H, dt, J = |
| | 6.8, 2.1 Hz), 7.72-7.80 (2H, m), 8.45 (1H, t, J = 5.9 Hz) |
| 1401 | 1.11 (3H, t, J = 7.2 Hz), 2.01-2.13 (2H, m), 2.37 (2H, t, J = 8.0 Hz), 3.20 (2H, t, J = 6.6 Hz), |
| | 3.23-3.30 (2H, m), 3.65 (2H, t, J = 7.0 Hz), 3.80 (3H, s), 4.02 (2H, t, J = 6.6 Hz), 7.11 (1H, d, |
| | J = 8.9 Hz), 7.24 (1H, d, J = 2.7 Hz), 7.29 (1H, dd, J = 8.8, 2.7 Hz), 7.46-7.56 (2H, m), 7.62 |
| | (1H, dt, J = 6.8, 2.1 Hz), 7.75-7.80 (1H, m), 8.45 (1H, t, J = 5.9 Hz) |
| 1402 | 0.57 (1H, td, J = 5.3, 2.2 Hz), 0.87 (1H, dt, J = 8.4, 5.4 Hz), 1.11 (3H, t, J = 7.2 Hz), 1.54- |
| | 1.65 (1H, m), 2.36 (1H, d, J = 18.1 Hz), 2.80 (1H, dd, J = 18.0, 7.3 Hz), 3.20 (2H, t, J = 6.6 |
| | Hz), 3.24-3.30 (2H, m), 3.38-3.46 (1H, m), 3.81 (3H, s), 4.02 (2H, t, J = 6.6 Hz), 7.13 (1H, |
| | d, J = 8.9 Hz), 7.24 (1H, d, J = 2.6 Hz), 7.31 (1H, dd, J = 8.9, 2.6 Hz), 7.46-7.56 (2H, m), |
| | 7.63 (1H, dt, J = 6.6, 2.1 Hz), 7.75-7.81 (1H, m), 8.46 (1H, t, J = 5.9 Hz) |
| 1403 | 2.94 (3H, s), 2.98 (2H, t, J = 6.6 Hz), 3.02 (2H, t, J = 6.7 Hz), 3.03 (3H, s), 3.52-3.58 (2H, |
| | m), 4.06 (2H, t, J = 6.6 Hz), 4.27 (2H, s), 7.32 (1H, d, J = 8.1 Hz), 7.43-7.50 (3H, m), 7.55 |
| | (1H, dt, J = 7.3, 2.0 Hz), 7.58 (1H, s), 7.65-7.70 (1H, m), 7.83 (1H, d, J = 2.3 Hz) |
| 1404 | 0.19-0.54 (4H, m), 1.04 (1H, dt, J = 8.8, 4.6 Hz), 2.98 (2H, t, J = 6.6 Hz), 3.03 (3H, s), 3.18- |
| | 3.25 (2H, m), 3.34-3.44 (1H, m), 3.46-3.66 (4H, m), 4.06 (2H, t, J = 6.6 Hz), 4.73(1H, t, J = |
| | 5.6 Hz), 7.32 (1H, d, J = 8.2 Hz), 7.45 (1H, dd, J = 8.1, 2.4 Hz), 7.50-7.56 (2H, m), 7.64 (1H, |
| | dt, J = 6.5, 2.2 Hz), 7.78-7.86 (2H, m), 8.03 (1H, d, J = 9.0 Hz) |
| 1405 | 1.33 (3H, t, J = 7.1 Hz), 2.63 (1H, dd, J = 17.4, 6.2 Hz), 2.92 (1H, dd, J = 17.4, 9.9 Hz), 3.21 |
| | (2H, t, J = 6.6 Hz), 3.49-3.62 (1H, m), 3.87-3.94 (1H, m), 4.07-4.16 (3H, m), 4.36 (2H, q, |
| | J = 7.1 Hz), 7.16-7.20 (1H, m), 7.41 (1H, t, J = 8.1 Hz), 7.49-7.61 (4H, m), 7.70 (1H, t, J = |
| | 2.1 Hz), 7.75 (1H, t, J = 1.9 Hz) |
| 1406 | 0.22-0.40 (3H, m), 0.42-0.51 (1H, m), 0.99-1.10 (1H, m), 2.98 (2H, t, J = 6.6 Hz), 3.03 |
| | (3H, s), 3.15-3.27 (2H, m), 3.34-3.43 (1H, m), 3.50-3.68 (4H, m), 4.06 (2H, t, J = 6.7 Hz), |
| | 4.73 (1H, t, J = 5.6 Hz), 7.32 (1H, d, J = 8.1 Hz), 7.45 (1H, dd, J = 8.1, 2.4 Hz), 7.48-7.55 |
| | (2H, m), 7.64 (1H, dt, J = 6.8, 2.1 Hz), 7.80 (1H, dt, J = 2.4, 1.2 Hz), 7.83 (1H, d, J = 2.4 Hz), |
| | 8.02 (1H, d, J = 9.0 Hz) |
| 1407 | 0.46 (1H, td, J = 5.3, 2.3 Hz), 1.04-1.08 (1H, m), 1.11 (3H, t, J = 7.2 Hz), 1.54-1.61 (1H, |
| | m), 2.46 (1H, d, J = 18.2 Hz), 2.96 (1H, dd, J = 18.2, 7.3 Hz), 3.21 (2H, t, J = 6.6 Hz), 3.28 |
| | (2H, dd, J = 7.3, 6.0 Hz), 3.54 (1H, t, J = 6.0 Hz), 4.08 (2H, t, J = 6.7 Hz), 7.13-7.17 (1H, m), |
| | 7.41 (1H, t, J = 8.1 Hz), 7.46-7.57 (3H, m), 7.63 (1H, dt, J = 6.8, 2.1 Hz), 7.69 (1H, t, J = 2.1 |
| | Hz), 7.77-7.80 (1H, m), 8.46 (1H, t, J = 5.9 Hz) |
| 1408 | 0.46 (1H, td, J = 5.2, 2.2 Hz), 1.03-1.08 (1H, m), 1.11 (3H, t, J = 7.2 Hz), 1.53-1.63 (1H, |
| | m), 2.46 (1H, d, J = 18.1 Hz), 2.96 (1H, dd, J = 18.2, 7.3 Hz), 3.21 (2H, t, J = 6.6 Hz), 3.28 |
| | (2H, dd, J = 7.4, 6.1 Hz), 3.55 (1H, t, J = 6.0 Hz), 4.08 (2H, t, J = 6.6 Hz), 7.12-7.17 (1H, m), |
| | 7.41 (1H, t, J = 8.1 Hz), 7.47-7.57 (3H, m), 7.63 (1H, dt, J = 6.8, 2.1 Hz), 7.69 (1H, t, J = 2.1 |
| | Hz), 7.79 (1H, t, J = 2.0 Hz), 8.46 (1H, t, J = 6.0 Hz) |
| 1409 | 1.07-1.15 (9H, m), 3.16-3.34 (4H, m) , 3.35-3.44 (2H, m) , 3.72-3.81 (2H, m) , 3.96- |
| | 4.09 (3H, m) , 6.97 (1H, d, J = 7.7 Hz), 7.32 (1H, t, J = 8.0 Hz), 7.37 (1H, d, J = 8.5 Hz), 7.46- |
| | 7.56 (2H, m), 7.57-7.67 (2H, m), 7.78 (1H, t, J = 2.1 Hz), 8.46 (1H, t, J = 5.9 Hz) |
| 1410 | 2.98 (2H, t, J = 6.6 Hz), 3.03 (3H, s), 3.22 (2H, t, J = 6.6 Hz), 3.26 (3H, s), 3.39-3.49 (4H, |
| | m), 3.55 (2H, t, J = 6.7 Hz), 4.06 (2H, t, J = 6.6 Hz), 7.32 (1H, d, J = 8.1 Hz), 7.45 (1H, dd, J = |
| | 8.1, 2.3 Hz), 7.48-7.55 (2H, m), 7.61-7.67 (1H, m), 7.79 (1H, dt, J = 2.0, 1.0 Hz), 7.83 (1H, |
| | d, J = 2.3 Hz), 8.36 (1H, t, J = 5.5 Hz) |
| 1411 | 2.98 (2H, t, J = 6.7 Hz), 3.03 (3H, s), 3.21 (2H, t, J = 6.6 Hz), 3.49-3.58 (3H, m), 3.63 (1H, |
| | dd, J = 8.9, 4.1 Hz), 3.91 (1H, dd, J = 9.3, 5.0 Hz), 3.99 (1H, dd, J = 9.0, 6.1 Hz), 4.06 (2H, |
| | t, J = 6.6 Hz), 4.19-4.26 (1H, m), 4.28 (1H, s), 5.28 (1H, s), 7.32 (1H, d, J = 8.2 Hz), 7.45 (1H, |
| | dd, J = 8.1, 2.4 Hz), 7.48-7.54 (2H, m), 7.65 (1H, dt, J = 6.5, 2.2 Hz), 7.80 (1H, t, J = 1.5 |
| | Hz), 7.83 (1H, d, J = 2.3 Hz), 8.44 (1H, d, J = 7.1 Hz) |
| 1412 | 2.98 (2H, t, J = 6.6 Hz), 3.03 (3H, s), 3.23 (2H, t, J = 6.6 Hz), 3.50-3.58 (3H, m), 3.62 (1H, |
| | dd, J = 9.6, 2.3 Hz), 3.90-4.00 (2H, m), 4.07 (2H, t, J = 6.6 Hz), 4.27 (1H, td, J = 5.2, 2.3 |
| | Hz), 4.28-4.37 (1H, m), 5.58-5.62 (1H, m), 7.32 (1H, d, J = 8.1 Hz), 7.45 (1H, dd, J = 8.0, |
| | 2.4 Hz), 7.48-7.57 (2H, m), 7.62 (1H, dt, J = 7.0, 2.0 Hz), 7.76-7.82 (2H, m), 7.83 (1H, |
| | d, J = 2.3 Hz) |
| 1413 | 1.11 (3H, t, J = 7.2 Hz), 1.44-1.60 (4H, m), 1.71-1.88 (2H, m), 2.19-2.31 (1H, m), 2.71- |
| | 2.83 (2H, m), 3.17-3.34 (4H, m), 4.07 (2H, t, J = 6.6 Hz), 4.68 (1H, t, J = 6.2 Hz), 7.12-7.22 |
| | (1H, m), 7.35-7.45 (2H, m), 7.47-7.55 (2H, m), 7.57-7.67 (2H, m), 7.78-7.80 (1H, m), |
| | 8.46 (1H, t, J = 5.9 Hz) |
| 1414 | 2.43 (3H, s), 2.95 (2H, t, J = 6.5 Hz), 3.21 (2H, t, J = 6.6 Hz), 3.50-3.64 (6H, m), 4.06 (2H, t, |
| | J = 6.6 Hz), 4.41 (2H, d, J = 6.2 Hz), 4.77 (1H, d, J = 5.0 Hz), 7.21 (1H, d, J = 7.9 Hz), 7.32 |
| | (1H, d, J = 8.1 Hz), 7.41-7.55 (3H, m), 7.59-7.67 (2H, m), 7.77-7.85 (2H, m), 8.41 (1H, d, |
| | J = 2.3 Hz), 9.07 (1H, t, J = 6.2 Hz) |
| 1415 | 2.98 (2H, t, J = 6.6 Hz), 3.03 (3H, s), 3.21 (2H, t, J = 6.6 Hz), 3.49-3.58 (3H, m), 3.63 (1H, |
| | dd, J = 8.9, 4.1 Hz), 3.91 (1H, dd, J = 9.3, 5.0 Hz), 3.99 (1H, dd, J = 9.0, 6.1 Hz), 4.06 (2H, |
| | t, J = 6.6 Hz), 4.21-4.30 (2H, m), 5.27 (1H, d, J = 4.3 Hz), 7.32 (1H, d, J = 8.2 Hz), 7.45 (1H, dd, |
| | J = 8.1, 2.4 Hz), 7.48-7.55 (2H, m), 7.65 (1H, dt, J = 6.9, 2.1 Hz), 7.77-7.85 (2H, m), 8.43 |
| | (1H, d, J = 7.1 Hz) |
| 1416 | 2.98 (2H, t, J = 6.6 Hz), 3.03 (3H, s), 3.23 (2H, t, J = 6.6 Hz), 3.50-3.58 (3H, m), 3.62 (1H, |
| | dd, J = 9.6, 2.3 Hz), 3.93 (1H, dd, J = 8.8, 3.7 Hz), 3.97 (1H, dd, J = 7.3, 6.3 Hz), 4.07 (2H, t, |
| | J = 6.6 Hz), 4.24-4.37 (2H, m), 5.60 (1H, d, J = 3.6 Hz), 7.32 (1H, d, J = 8.2 Hz), 7.45 (1H, dd, |
| | J = 8.1, 2.4 Hz), 7.48-7.56 (2H, m), 7.62 (1H, dt, J = 6.7, 2.1 Hz), 7.76-7.86 (3H, m) |
| 1417 | 1.67 (2H, p, J = 6.6 Hz), 2.97 (2H, t, J = 6.6 Hz), 3.03 (3H, s), 3.22 (2H, t, J = 6.6 Hz), 3.28- |
| | 3.36 (2H, m), 3.46 (2H, td, J = 6.2, 5.1 Hz), 3.55 (2H, t, J = 6.7 Hz), 4.06 (2H, t, J = 6.6 Hz), |
| | 4.49 (1H, t, J = 5.2 Hz), 7.32 (1H, d, J = 8.2 Hz), 7.45 (1H, dd, J = 8.1, 2.4 Hz), 7.47-7.55 |
| | (2H, m), 7.61-7.66 (1H, m), 7.79 (1H, dt, J = 2.1, 1.0 Hz), 7.83 (1H, d, J = 2.3 Hz), 8.41 (1H, |
| | t, J = 5.9 Hz) |
| 1418 | 1.11 (3H, t, J = 7.1 Hz), 3.09 (3H, s), 3.18 (2H, t, J = 6.5 Hz), 3.25-3.29 (2H, m), 3.61 (2H, t, |
| | J = 4.8 Hz), 4.28 (2H, t, J = 6.5 Hz), 4.41-4.47 (2H, m), 6.68 (1H, s), 7.53-7.55 (1H, m), |
| | 7.63-7.66 (1H, m), 7.79-7.84 (1H, m), 7.91 (1H, s), 8.25 (1H, s), 8.47 (1H, t, J = 5.9 Hz) |
| 1419 | 1.11 (3H, t, J = 7.2 Hz), 1.32-1.40 (2H, m), 1.44-1.52 (4H, m), 2.27-2.40 (4H, m), 3.19 |
| | (2H, t, J = 6.7 Hz), 3.26-3.28 (2H, m), 3.40 (2H, s), 3.77 (3H, s), 4.00 (2H, t, J = 6.6 Hz), |
| | 6.57 (1H, s), 6.98 (1H, d, J = 8.9 Hz), 7.20 (1H, dd, J = 8.7, 2.7 Hz), 7.27 (1H, d, J = 2.7 Hz), |
| | 7.50-7.52 (1H, m), 7.60-7.65 (1H, m), 7.75-7.81 (1H, m), 8.44 (1H, t, J = 5.9 Hz) |
| 1420 | 1.11 (3H, t, J = 7.1 Hz), 2.27 (3H, s), 2.68-2.74 (2H, m), 3.10 (2H, s), 3.21 (2H, t, J = 6.6 |
| | Hz), 3.26-3.28 (2H, m), 3.65 (2H, dd, J = 6.3, 4.5 Hz), 4.07 (2H, t, J = 6.6 Hz), 7.19-7.23 |
| | (1H, m), 7.24-7.31 (1H, m), 7.36 (1H, t, J = 2.1 Hz), 7.41 (1H, t, J = 8.0 Hz), 7.47-7.55 (2H, |
| | m), 7.60-7.65 (1H, m), 7.78 (1H, t, J = 2.0 Hz), 8.45 (1H, t, J = 5.9 Hz) |
| 1421 | 0.56 (4H, s), 2.98 (2H, t, J = 6.6 Hz), 3.03 (3H, s), 3.22 (2H, t, J = 6.6 Hz), 3.42 (2H, d, J = 5.9 |
| | Hz), 3.55 (2H, t, J = 6.7 Hz), 4.07 (2H, t, J = 6.6 Hz), 5.47 (1H, s), 7.32 (1H, d, J = 8.2 Hz), |
| | 7.45 (1H, dd, J = 8.0, 2.3 Hz), 7.48-7.55 (2H, m), 7.61-7.68 (1H, m), 7.79-7.82 (1H, m), |
| | 7.83 (1H, d, J = 2.3 Hz), 8.21 (1H, t, J = 5.9 Hz) |
| 1422 | 1.11 (3H, t, J = 7.2 Hz), 3.16 (2H, t, J = 6.6 Hz), 3.22-3.33 (4H, m), 3.93 (2H, t, J = 6.7 Hz), |
| | 4.10 (2H, t, J = 4.4 Hz), 5.85 (1H, s), 6.42 (1H, dd, J = 8.4, 2.4 Hz), 6.53 (1H, d, J = 2.5 Hz), |
| | 6.63 (1H, d, J = 8.4 Hz), 7.45-7.56 (2H, m), 7.61 (1H, m), 7.74-7.79 (1H, m), 8.43 (1H, |
| | t, J = 5.9 Hz) |
| 1423 | 1.11 (3H, t, J = 7.2 Hz), 2.21 (3H, s), 2.43 (4H, t, J = 5.0 Hz), 3.12 (4H, dd, J = 5.1, 5.1 Hz), |
| | 3.18 (2H, t, J = 6.6 Hz), 3.23-3.30 (2H, m), 4.02 (2H, t, J = 6.6 Hz), 6.73-6.76 (1H, m ), |
| | 6.83 (1H, dd, J = 8.2, 2.4 Hz), 6.90 (1H, dd, J = 2.2, 2.2 Hz), 7.21 (1H, dd, J = 8.1, 8.1 Hz), |
| | 7.46-7.56 (2H, m), 7.60-7.63 (1H, m), 7.74-7.80 (1H, m), 8.45 (1H, t, J = 5.9 Hz) |
| 1424 | 1.69 (1H, s), 1.70-1.78 (2H, m), 2.33 (1H, s), 2.53 (1H, d, J = 11.1 Hz), 2.68-2.82 (2H, m), |
| | 2.91 (2H, t, J = 6.6 Hz), 2.96 (3H, s), 3.04 (1H, s), 3.09-3.23 (2H, m), 3.48 (2H, t, J = 6.7 |
| | Hz), 4.00 (2H, dd, J = 10.3, 6.4 Hz), 4.38 (1H, s), 7.25 (1H, d, J = 8.2 Hz), 7.38 (1H, dd, J = |
| | 8.0, 2.4 Hz), 7.41-7.49 (2H, m), 7.60 (1H, dt, J = 6.9, 2.0 Hz), 7.74-7.79 (2H, m), 8.18 (1H, |
| | d, J = 9.3 Hz) |
| 1425 | 2.98 (3H, t, J = 6.7 Hz), 3.03 (3H, s), 3.15-3.29 (4H, m), 3.55 (2H, t, J = 6.7 Hz), 4.01-4.14 |
| | (2H, m), 4.59 (1H, dt, J = 16.1, 7.9 Hz), 7.32 (1H, d, J = 8.1 Hz), 7.45 (1H, dd, J = 8.1, 2.3 |
| | Hz), 7.48-7.57 (2H, m), 7.64-7.69 (1H, m), 7.78-7.85 (2H, m), 8.52 (1H, d, J = 9.0 Hz) |
| 1426 | 1.11 (3H, t, J = 7.2 Hz), 3.18-3.30 (4H, m), 3.70-3.76 (2H, m), 3.92-4.00 (2H, m), 4.08 |
| | (2H, t, J = 6.6 Hz), 4.19 (2H, s), 7.24-7.32 (2H, m), 7.39-7.45 (2H, m), 7.47-7.55 (2H, m), |
| | 7.60-7.65 (1H, m), 7.76-7.80 (1H, m), 8.46 (1H, t, J = 5.9 Hz) |
| 1427 | 0.39-0.44 (2H, m), 0.49-0.55 (2H, m), 2.08 (2H, broad s), 2.98 (2H, t, J = 6.6 Hz), 3.03 |
| | (3H, s), 3.22 (2H, t, J = 6.6 Hz), 3.29 (2H, d, J = 6.0 Hz), 3.55 (2H, t, J = 6.7 Hz), 4.06 (2H, |
| | t, J = 6.6 Hz), 7.32 (1H, d, J = 8.1 Hz), 7.45 (1H, dd, J = 8.1, 2.3 Hz), 7.48-7.56 (2H, m), 7.63- |
| | 7.68 (1H, m), 7.80 (1H, d, J = 2.0 Hz), 7.83 (1H, d, J = 2.3 Hz), 8.36 (1H, t, J = 6.0 Hz) |
| 1428 | 1.11 (3H, t, J = 7.2 Hz), 2.91 (2H, s), 3.21 (2H, t, J = 6.6 Hz), 3.27-3.30 (2H, m), 4.06 (2H, t, |
| | J = 6.6 Hz), 4.12 (2H, s), 4.55-4.64 (4H, m), 7.11-7.16 (1H, m), 7.39 (1H, dd, J = 8.1, 8.1 |
| | Hz), 7.47-7.54 (3H, m), 7.60-7.64 (1H, m), 7.66 (1H, dd, J = 2.1, 2.1 Hz), 7.76-7.80 (1H, |
| | m), 8.45 (1H, t, J = 5.9 Hz) |
| 1429 | 1.01-1.07 (2H, m), 1.09-1.13 (5H, m), 2.12 (3H, s), 3.19 (2H, t, J = 6.5 Hz), 3.26-3.28 |
| | (2H, m), 3.99 (2H, t, J = 6.6 Hz), 4.13 (2H, s), 6.80 (1H, d, J = 8.0 Hz), 6.93 (1H, dd, J = 8.0, |
| | 2.0 Hz), 7.46-7.54 (2H, m), 7.59-7.64 (1H, m), 7.77 (1H, d, J = 2.2 Hz), 7.98 (1H, d, J = 2.0 |
| | Hz), 8.43 (1H, t, J = 5.9 Hz) |
| 1430 | 0.57-0.65 (4H, m), 1.11 (3H, t, J = 7.1 Hz), 1.89 (2H, t, J = 6.8 Hz), 3.13 (2H, s), 3.18 (2H, t, |
| | J = 6.6 Hz), 3.25-3.40 (4H, m), 4.02 (2H, t, J = 6.6 Hz), 6.37-6.43 (1H, m), 6.45 (1H, t, J = |
| | 2.1 Hz), 6.53-6.59 (1H, m), 7.16 (1H, t, J = 8.0 Hz), 7.48-7.54 (2H, m), 7.58-7.64 (1H, |
| | m), 7.74-7.79 (1H, m), 8.44 (1H, t, J = 5.9 Hz) |
| 1431 | 0.24 (1H, q, J = 4.1 Hz), 0.69 (1H, dt, J = 7.7, 4.3 Hz), 1.11 (3H, t, J = 7.1 Hz), 1.64-1.70 |
| | (2H, m), 3.11-3.22 (4H, m), 3.45 (2H, d, J = 9.2 Hz), 4.00 (2H, t, J = 6.6 Hz), 6.40-6.47 |
| | (1H, m), 6.47-6.52 (1H, m), 6.54-6.61 (1H, m), 7.14 (1H, t, J = 8.0 Hz), 7.46-7.56 (2H, |
| | m), 7.56-7.65 (1H, m), 7.74-7.79 (1H, m), 8.43 (1H, t, J = 5.9 Hz). 2H overlapping with |
| | water peak not resolved |
| 1432 | 1.12 (3H, t, J = 7.1 Hz), 2.89 (3H, s), 3.20 (2H, t, J = 6.6 Hz), 3.37-3.51 (4H, m), 3.76 (2H, |
| | s), 4.03 (2H, t, J = 6.6 Hz), 6.75-6.81 (1H, m), 6.81-6.88 (1H, m), 6.91-6.96 (1H, m), 7.24 |
| | (1H, t, J = 8.1 Hz), 7.46-7.57 (2H, m), 7.57-7.66 (1H, m), 7.75-7.80 (1H, m), 8.45 (1H, t, |
| | J = 5.9 Hz). 2H overlapping with water peak not resolved |
| 1433 | 0.49 (1H, td, J = 5.2, 2.2 Hz), 1.03-1.09 (1H, m), 1.11 (3H, t, J = 7.2 Hz), 1.54-1.64 (1H, |
| | m), 2.96 (1H, dd, J = 18.3, 7.3 Hz), 3.22 (2H, t, J = 6.6 Hz), 3.26-3.30 (3H, m), 3.54-3.58 |
| | (1H, m), 4.02 (2H, t, J = 6.7 Hz), 7.34 (1H, t, J = 9.0 Hz), 7.50-7.56 (2H, m), 7.59-7.65 (2H, |
| | m), 7.75-7.81 (2H, m), 8.47 (1H, t, J = 5.9 Hz) |
| 1434 | 1.12 (3H, t, J = 7.2 Hz), 1.57-1.64 (2H, m), 2.84-2.95 (2H, m), 3.14 (3H, s), 3.20-3.28 |
| | (4H, m), 3.47-3.55 (1H, m), 4.02-4.15 (3H, m), 7.25-7.31 (1H, m), 7.37 (1H, dd, J = 8.0, |
| | 2.4 Hz), 7.49-7.52 (2H, m), 7.63 (1H, dt, J = 6.5, 2.3 Hz), 7.78-7.80 (1H, m), 8.39 (1H, |
| | d, J = 2.4 Hz), 8.44 (1H, t, J = 5.9 Hz) |
| 1435 | 1.11 (3H, t, J = 7.2 Hz), 3.08 (3H, s), 3.20 (2H, d, J = 6.7 Hz), 3.27 (2H, s), 3.60 (2H, t, J = 5.0 |
| | Hz), 3.98 (2H, s), 4.42 (2H, t, J = 4.8 Hz), 7.00 (1H, d, J = 11.0 Hz), 7.52 (2H, d, J = 6.5 Hz), |
| | 7.59-7.66 (1H, m), 7.80 (2H, d, J = 9.0 Hz), 8.42-8.50 (1H, m) |
| 1436 | 1.11 (3H, t, J = 7.2 Hz), 1.77-1.93 (4H, m), 2.06 (3H, s), 2.26 (2H, d, J = 10.6 Hz), 2.44 (2H, |
| | dd, J = 10.9, 2.4 Hz), 3.18 (2H, t, J = 6.6 Hz), 3.22-3.32 (2H, m), 4.01 (2H, t, J = 6.6 Hz), |
| | 4.19 (2H, s), 6.59 (1H, dd, J = 7.5, 1.8 Hz), 6.70 (1H, dd, J = 8.2, 2.3 Hz), 6.74-6.80 (1H, m), |
| | 7.15 (1H, t, J = 8.1 Hz), 7.46-7.56 (2H, m), 7.61 (1H, dt, J = 6.6, 2.1 Hz), 7.74-7.80 (1H, |
| | m), 8.44 (1H, t, J = 5.9) |
| 1437 | 2.12 (6H, s), 2.33 (3H, s), 2.59 (2H, t, J = 5.9 Hz), 2.80 (2H, t, J = 4.8 Hz), 3.17 (2H, t, J = 6.6 |
| | Hz), 3.46 (2H, s), 4.02 (2H, t, J = 6.6 Hz), 6.22 (1H, s), 7.02-7.06 (1H, m), 7.12 (2H, d, J = |
| | 1.8 Hz), 7.49-7.54 (2H, m), 7.63 (1H, dt, J = 6.9, 2.1 Hz), 7.78 (1H, td, J = 1.9, 0.6 Hz), 8.89 |
| | (1H, s) |
| 1438 | 0.55 (1H, dd, J = 8.2, 3.0 Hz), 0.64-0.72 (1H, m), 0.76 (2H, t, J = 2.3 Hz), 2.98 (2H, t, J = |
| | 6.6Hz), 3.03 (3H, s), 3.19 (2H, tt, J = 7.0, 3.2 Hz), 3.50-3.58 (3H, m), 3.83 (1H, dd, J = 9.0, |
| | 4.7 Hz), 3.89 (1H, d, J = 8.1 Hz), 4.03-4.09 (2H, m), 4.11 (1H, dd, J = 8.9, 6.6 Hz), 4.32 (1H, |
| | ddd, J = 8.4, 6.5, 4.7 Hz), 7.32 (1H, d, J = 8.1 Hz), 7.45 (1H, dd, J = 8.1, 2.3Hz), 7.48-7.57 |
| | (2H, m), 7.67 (1H, dt, J = 6.8, 2.1 Hz), 7.80-7.85 (2H, m), 8.36 (1H, d, J = 8.3 Hz) 1H |
| | overlapping with water peak not observed |
| 2001 | (CD3OD) 2.47 (3H, s), 2.79 (2H, t, J = 6.1 Hz), 2.95 (2H, t, J = 6.0 Hz), 3.65 (2H, s), 3.86 (2H, |
| | d, J = 6.2 Hz), 4.09 (2H, t, J = 6.8 Hz), 5.18 (1H, t, J = 6.1 Hz), 7.05-7.10 (1H, m), 7.13 (1H, |
| | dd, J = 8.2, 2.2 Hz), 7.18 (1H, s), 7.23-7.29 (1H, m), 7.30-7.38 (3H, m), 7.38-7.45 (2H, |
| | m), 7.59 (1H, ddd, J = 8.1, 6.5, 1.6 Hz), 7.64 (1H, ddd, J = 8.3, 6.8, 1.6 Hz) 2 protons |
| | masked by CD3OD peak |
| 2002 | 2.34 (3H, s), 2.51-2.55 (2H, m), 2.65-2.70 (2H, m), 2.76 (2H, t, J = 5.8 Hz), 3.21 (2H, t, J = |
| | 6.7 Hz), 3.55 (4H, d, J = 5.4 Hz), 4.03 (2H, t, J = 6.7 Hz), 4.45 (3H, d, J = 6.2 Hz), 7.00 (1H, d, |
| | J = 2.0 Hz), 7.03-7.11 (2H, m), 7.16-7.19 (1H, m), 7.37 (1H, td, J = 8.2, 1.4 Hz), 7.65 (1H, |
| | ddd, J = 8.3, 6.7, 1.6 Hz), 7.76 (1H, ddd, J = 8.4, 6.8, 1.6 Hz), 8.29-8.31 (1H, m), 8.41 (1H, |
| | s), 9.03 (1H, t, J = 6.0 Hz) |
| 2004 | 0.17 (s, 2H), 0.35-0.45 (m, 2H), 1.00-1.10 (m, 1H), 2.96 (t, J = 6.7 Hz, 2H), 3.02 (s, 3H), |
| | 3.10 (t, J = 6.5 Hz, 2H), 3.25 (t, J = 6.7 Hz, 2H), 3.53 (t, J = 6.6 Hz, 2H), 4.09 (t, J = 6.6 Hz, |
| | 2H), 7.31 (d, J = 8.2 Hz, 1H), 7.39 (td, J = 8.1, 1.4 Hz, 1H), 7.43 (dd, J = 8.1, 2.4 Hz, 1H), |
| | 7.67 (ddd, J = 8.2, 6.7, 1.7 Hz, 1H), 7.74-7.83 (m, 2H), 8.57 (t, J = 6.0 Hz, 1H). |
| 2005 | 0.15-0.30 (2H, m), 0.35-0.50 (2H, m), 0.98-1.15 (1H, m), 2.97 (2H, t, J = 6.7 Hz), 3.02 |
| | (3H, s), 3.12 (2H, t, J = 6.5 Hz), 3.22 (2H, t, J = 6.6 Hz), 3.54 (2H, t, J = 6.7 Hz), 4.06 (2H, t, |
| | J = 6.6 Hz), 7.32 (1H, d, J = 8.2 Hz), 7.46 (1H, dd, J = 8.1, 2.3 Hz), 7.70 (1H, t, J = 8.0 Hz), 7.83 |
| | (1H, d, J = 2.3 Hz), 7.93 (1H, dt, J = 7.8, 1.2 Hz), 8.04 (1H, ddd, J = 8.2, 2.2, 1.1 Hz), 8.24 |
| | (1H, t, J = 2.3 Hz), 8.58 (1H, t, J = 6.0 Hz) |
| 2006 | 0.17-0.30 (2H, m), 0.33-0.49 (2H, m), 0.98-1.13 (1H, m), 2.97 (2H, t, J = 6.6 Hz), 3.02 |
| | (3H, s), 3.12 (2H, t, J = 6.3 Hz), 3.24 (2H, t, J = 6.3 Hz), 3.54 (2H, t, J = 6.7 Hz), 4.08 (2H, |
| | t, J = 6.6 Hz), 7.31 (1H, d, J = 8.2 Hz), 7.45 (1H, dd, J = 8.1, 2.4 Hz), 7.50-7.61 (2H, m), 7.75- |
| | 7.80 (1H, m), 7.81 (1H, d, J = 2.3 Hz), 7.95 (1H, d, J = 8.0 Hz), 8.05-8.14 (2H, m), 8.52 (1H, |
| | t, J = 5.4 Hz) |
| 2007 | 2.36 (3H, s), 2.37 (3H, s), 2.54 (2H, t, J = 6.2 Hz), 2.69 (2H, t, J = 5.7 Hz), 2.78 (2H, t, J = 5.7 |
| | Hz), 3.18 (2H, t, J = 6.6 Hz), 3.53-3.62 (4H, m), 4.01 (2H, t, J = 6.6 Hz), 4.42-4.50 (3H, m), |
| | 7.02 (1H, s), 7.09 (2H, s), 7.18 (1H, d, J = 4.9 Hz), 7.44 (1H, d, J = 8.3 Hz), 7.52 (1H, dd, J = |
| | 8.2, 2.2 Hz), 7.74 (1H, d, J = 2.2 Hz), 8.32 (1H, d, J = 4.9 Hz), 8.43 (1H, s), 8.96 (1H, t, J = 6.0 |
| | Hz) |
| 2008 | 2.34 (3H, s), 2.36 (3H, s), 2.54 (2H, t, J = 6.9 Hz), 2.69 (2H, t, J = 5.7 Hz), 2.78 (2H, t, J = 5.7 |
| | Hz), 3.19 (2H, t, J = 6.6 Hz), 3.49-3.66 (4H, m), 4.01 (2H, t, J = 6.6 Hz), 4.40-4.53 (3H, m), |
| | 7.02 (1H, s), 7.09 (2H, s), 7.18 (1H, d, J = 4.9 Hz), 7.23-7.29 (1H, m), 7.31-7.42 (3H, m), |
| | 8.31 (1H, d, J = 4.9 Hz), 8.43 (1H, s), 8.93 (1H, t, J = 6.0 Hz) |
| 2009 | 2.36 (3H, s), 2.54 (2H, t, J = 6.2 Hz), 2.69 (2H, t, J = 5.8 Hz), 2.77 (2H, t, J = 5.7 Hz), 3.20 |
| | (2H, t, J = 6.6 Hz), 3.52-3.61 (4H, m), 4.03 (2H, t, J = 6.6 Hz), 4.41-4.51 (3H, m), 7.03 (1H, |
| | s), 7.10 (2H, s), 7.19 (1H, d, J = 4.9 Hz), 7.72 (1H, t, J = 7.9 Hz), 7.82 (1H, d, J = 7.6 Hz), 7.96 |
| | (1H, d, J = 8.3 Hz), 8.04 (1H, s), 8.32 (1H, d, J = 4.9 Hz), 8.43 (1H, s), 9.01 (1H, t, J = 6.0 Hz) |
| 2010 | 2.36 (3H, s), 2.52-2.59 (2H, m), 2.69 (2H, t, J = 5.5 Hz), 2.78 (2H, t, J = 5.7 Hz), 3.20 (2H, t, |
| | J = 6.6 Hz), 3.55-3.59 (4H, m), 4.02 (2H, t, J = 6.6 Hz), 4.43-4.51 (3H, m), 7.03 (1H, s), |
| | 7.11 (2H, s), 7.19 (1H, d, J = 5.0 Hz), 7.69 (1H, t, J = 8.0 Hz), 7.92 (1H, dt, J = 7.8, 1.4 Hz), |
| | 8.01 (1H, ddd, J = 8.2, 2.2, 1.1 Hz), 8.21 (1H, t, J = 1.9 Hz), 8.32 (1H, d, J = 4.9 Hz), 8.44 |
| | (1H, s), 9.00 (1H, t, J = 6.0 Hz) |
| 2011 | 0.19-0.27 (2H, m), 0.37-0.46 (2H, m), 0.99-1.10 (1H, m), 2.35 (3H, s), 2.97 (2H, t, J = |
| | 6.6 Hz), 3.02 (3H, s), 3.11 (2H, t, J = 6.5 Hz), 3.22 (2H, t, J = 6.6 Hz), 3.54 (2H, t, J = 6.5 Hz), |
| | 4.06 (2H, t, J = 6.6 Hz), 7.22-7.48 (6H, m), 7.81 (1H, d, J = 2.3 Hz), 8.47 (1H, t, J = 6.0 Hz) |
| 2012 | 2.19 (3H, s), 2.98 (2H, t, J = 6.6 Hz), 3.03 (3H, s), 3.23 (2H, t, J = 6.6 Hz), 3.55 (2H, t, J = 6.7 |
| | Hz), 3.65 (3H, s), 4.07 (2H, t, J = 6.6 Hz), 4.31 (2H, d, J = 6.0 Hz), 5.94 (1H, s), 7.32 (1H, d, J = |
| | 8.1 Hz), 7.46 (1H, dd, J = 8.1, 2.3 Hz), 7.69 (1H, t, J = 8.0 Hz), 7.84 (1H, d, J = 2.4 Hz), 7.92 |
| | (1H, dt, J = 7.8, 1.2 Hz), 8.04 (1H, ddd, J = 8.2, 2.2, 1.1 Hz), 8.24 (1H, t, J = 1.8 Hz), 8.72 |
| | (1H, t, J = 6.1 Hz) |
| 2013 | 0.16-0.32 (2H, m), 0.38-0.47 (2H, m), 0.98-1.10 (1H, m), 2.98 (2H, t, J = 6.6 Hz), 3.03 |
| | (3H, s), 3.13 (2H, t, J = 6.5 Hz), 3.22 (2H, t, J = 6.6 Hz), 3.55 (2H, t, J = 6.6 Hz), 4.06 (2H, t, |
| | J = 6.6 Hz), 7.33 (1H, d, J = 8.2 Hz), 7.46 (1H, dd, J = 8.0, 2.4 Hz), 7.85 (1H, d, J = 2.3 Hz), |
| | 8.16 (1H, t, J = 1.7 Hz), 8.27 (2H, dt, J = 13.3, 1.9 Hz), 8.60 (1H, t, J = 6.0 Hz) |
| 2014 | 0.20-0.28 (2H, m), 0.37-0.46 (2H, m), 0.98-1.14 (1H, m), 2.97 (2H, t, J = 6.6 Hz), 3.03 |
| | (3H, s), 3.12 (2H, t, J = 6.5 Hz), 3.22 (2H, t, J = 6.5 Hz), 3.55 (2H, t, J = 6.6 Hz), 4.08 (2H, t, |
| | J = 6.6 Hz), 7.32 (1H, d, J = 8.2 Hz), 7.45 (1H, dd, J = 8.1, 2.4 Hz), 7.69 (1H, s), 7.83 (1H, d, J = |
| | 2.3 Hz), 8.00 (2H, d, J = 1.7 Hz), 8.12 (1H, t, J = 1.7 Hz), 8.19 (1H, s), 8.57 (1H, t, J = 6.0 Hz) |
| 2015 | 0.20-0.28 (2H, m), 0.36-0.47 (2H, m), 0.97-1.11 (1H, m), 2.98 (2H, t, J = 6.6 Hz), 3.03 |
| | (3H, s), 3.12 (2H, t, J = 6.4 Hz), 3.21 (2H, t, J = 6.6 Hz), 3.55 (2H, t, J = 6.7 Hz), 4.03 (2H, t, |
| | J = 6.6 Hz), 7.33 (1H, d, J = 8.1 Hz), 7.47 (1H, dd, J = 8.1, 2.4 Hz), 7.54 (1H, d, J = 1.9 Hz), |
| | 7.80-7.86 (2H, m), 8.51 (1H, t, J = 6.1 Hz) |
| 2016 | 0.16-0.30 (2H, m), 0.35-0.46 (2H, m), 0.97-1.11 (1H, m), 2.97 (2H, t, J = 6.6 Hz), 3.02 |
| | (3H, s), 3.12 (2H, t, J = 6.4 Hz), 3.22 (2H, t, J = 6.5 Hz), 3.54 (2H, t, J = 6.6 Hz), 4.09 (2H, t, |
| | J = 6.5 Hz), 7.32 (1H, d, J = 8.2 Hz), 7.45 (1H, dd, J = 8.1, 2.4 Hz), 7.71 (1H, dd, J = 5.4, 1.9 |
| | Hz), 7.82 (1H, d, J = 2.4 Hz), 7.98 (1H, d, J = 1.8 Hz), 8.55 (2H, dd, J = 10.5, 5.7 Hz) |
| 2017 | 2.19 (3H, s), 2.97 (2H, t, J = 6.7 Hz), 3.02 (3H, s), 3.21 (2H, t, J = 6.6 Hz), 3.55 (2H, t, J = 6.7 |
| | Hz), 3.64 (3H, s), 3.80 (2H, s), 4.07 (2H, t, J = 6.6 Hz), 4.31 (2H, d, J = 6.1 Hz), 5.94 (1H, s), |
| | 7.32 (1H, d, J = 8.2 Hz), 7.45 (1H, dd, J = 8.1, 2.4 Hz), 7.52 (1H, t, J = 1.8 Hz), 7.59 (1H, t, J = |
| | 1.7 Hz), 7.64 (1H, t, J = 2.0 Hz), 7.82 (1H, d, J = 2.3 Hz), 8.31 (1H, s), 8.64 (1H, t, J = 6.1 Hz) |
| | 2 NH and 1 formic acid OH protons not observed |
| 2018 | 0.20-0.28 (2H, m), 0.34-0.46 (2H, m), 0.99-1.14 (1H, m), 2.97 (2H, t, J = 6.6 Hz), 3.03 |
| | (3H, s), 3.12 (2H, t, J = 6.5 Hz), 3.21 (2H, t, J = 6.6 Hz), 3.55 (2H, t, , J = 6.7 Hz), 3.79 (2H, s), |
| | 4.06 (2H, t, J = 6.6 Hz), 7.32 (1H, d, J = 8.2 Hz), 7.45 (1H, dd, J = 8.1, 2.3 Hz), 7.52 (1H, s), |
| | 7.58 (1H, s), 7.62 (1H, t, J = 2.0 Hz), 7.82 (1H, d, J = 2.3 Hz), 8.35 (1H, s), 8.50 (1H, t, J = 6.0 |
| | Hz) |
| | NH₂ obscured by water peak |
| 2019 | 0.19-0.28 (2H, m), 0.33-0.46 (2H, m), 0.98-1.12 (1H, m), 2.97 (2H, t, J = 6.6 Hz), 3.02 |
| | (3H, s), 3.12 (2H, t, J = 6.5 Hz), 3.23 (2H, t, J = 6.6 Hz), 3.54 (2H, t, J = 6.7 Hz), 3.82 (2H, s), |
| | 4.07 (2H, t, J = 6.6 Hz), 7.31 (1H, d, J = 8.2 Hz), 7.36-7.49 (4H, m), 7.59 (1H, s), 7.81 (1H, |
| | d, J = 2.3 Hz), 8.36 (1H, s), 8.45 (1H, t, J = 6.0 Hz) NH₂ obscured by water peak |
| 2020 | 0.19-0.28 (2H, m), 0.35-0.46 (2H, m), 0.98-1.11 (1H, m), 2.97 (2H, t, J = 6.7 Hz), 3.02 |
| | (3H, s), 3.11 (2H, t, J = 6.4 Hz), 3.23 (2H, t, J = 6.6 Hz), 3.54 (2H, t, J = 6.7 Hz), 4.10 (2H, t, |
| | J = 6.6 Hz), 7.32 (1H, d, J = 8.1 Hz), 7.45 (1H, dd, J = 8.1, 2.3 Hz), 7.70 (1H, dd, J = 7.9, 0.7 |
| | Hz), 7.76 (1H, dd, J = 7.9, 0.7 Hz), 7.82 (1H, d, J = 2.3 Hz), 8.10 (1H, t, J = 7.9 Hz), 8.58 (1H, |
| | t, J = 6.0 Hz) |
| 2021 | 0.18-0.30 (2H, m), 0.35-0.49 (2H, m), 0.94-1.14 (1H, m), 2.98 (2H, t, J = 6.7 Hz), 3.03 |
| | (3H, s), 3.13 (2H, t, J = 6.4 Hz), 3.23 (2H, t, J = 6.6 Hz), 3.55 (2H, t, J = 6.7 Hz), 4.07 (2H, t, |
| | J = 6.6 Hz), 7.33 (1H, d, J = 8.1 Hz), 7.46 (1H, dd, J = 8.0, 2.4 Hz), 7.84 (1H, d, J = 2.3 Hz), |
| | 8.40 (1H, t, J = 2.2 Hz), 8.59 (1H, t, J = 6.0 Hz), 8.72 (1H, d, J = 2.2 Hz), 8.89 (1H, d, J = 2.1 |
| | Hz) |
| 2022 | 1.33 (3H, t, J = 7.9 Hz), 2.32 (3H, s), 2.97 (2H, t, J = 6.6 Hz), 3.03 (3H, s), 3.21 (2H, t, J = 6.6 |
| | Hz), 3.55 (2H, t, J = 6.7 Hz), 3.74 (2H, s), 4.10 (2H, t, J = 6.6 Hz), 4.35 (2H, q, J = 7.1 Hz), |
| | 7.23 (2H, d, J = 9.6 Hz), 7.28-7.37 (2H, m), 7.44 (1H, dd, J = 8.0, 2.3 Hz), 7.81 (1H, d, J = |
| | 2.3 Hz) NH2 obscured by water peak |
| 2023 | 1.33 (3H, t, J = 7.1 Hz), 2.48 (3H, s), 2.96 (2H, t, J = 6.6 Hz), 3.02 (3H, s), 3.21 (2H, t, J = 6.6 |
| | Hz), 3.53 (2H, t, J = 6.5 Hz), 4.11 (2H, t, J = 6.7 Hz), 4.35 (2H, q, J = 7.1 Hz), 7.31 (1H, d, J = |
| | 8.2 Hz), 7.38-7.49 (3H, m), 7.80 (1H, d, J = 2.3 Hz), 7.89 (1H, t, J = 7.8 Hz) |
| 2024 | 1.11 (3H, t, J = 7.2 Hz), 2.33 (3H, s), 2.97 (2H, t, J = 6.6 Hz), 3.02 (3H, s), 3.21 (2H, t, J = 6.6 |
| | Hz), 3.24-3.31 (2H, m), 3.54 (2H, t, J = 6.7 Hz), 3.82 (2H, s), 4.07 (2H, t, J = 6.6 Hz), 7.21- |
| | 7.34 (3H, m), 7.39 (1H, s), 7.44 (1H, dd, J = 8.1, 2.4 Hz), 7.80 (1H, d, J = 2.3 Hz), 8.32 (1H, |
| | s), 8.38 (1H, t, J = 5.9 Hz) NH2 not observed |
| 2025 | 2.26 (3H, s), 2.94 (2H, t, J = 6.7 Hz), 3.01 (3H, s), 3.15 (2H, s), 3.52 (2H, t, J = 6.7 Hz), 3.96- |
| | 4.13 (4H, m), 6.82 (1H, s), 7.09 (1H, s), 7.21-7.31 (2H, m), 7.39 (1H, dd, J = 8.1, 2.3 Hz), |
| | 7.76 (1H, d, J = 2.3 Hz), 7.92 (1H, s) NH2 not observed |
| 2027 | 1.12 (3H, t, J = 7.2 Hz), 2.36 (3H, d, J = 0.8 Hz), 2.97 (2H, t, J = 6.7 Hz), 3.03 (3H, s), 3.23 |
| | (2H, t, J = 6.6 Hz), 3.25-3.30 (2H, m), 3.54 (2H, t, J = 6.7 Hz), 4.07 (2H, t, J = 6.7 Hz), 7.31 |
| | (1H, d, J = 8.1 Hz), 7.45 (1H, dd, J = 8.1, 2.4 Hz), 7.83 (1H, d, J = 2.3 Hz), 7.91 (1H, s), 8.44 |
| | (1H, t, J = 6.0 Hz), 8.47 (1H, dd, J = 1.9, 0.9 Hz), 8.63-8.67 (1H, m) |
| 2028 | 1.11 (3H, t, J = 7.1 Hz), 2.38 (3H, s), 2.98 (2H, t, J = 6.6 Hz), 3.03 (3H, s), 3.15-3.29 (4H, |
| | m), 3.55 (2H, t, J = 6.7 Hz), 4.09 (2H, t, J = 6.6 Hz), 7.31 (1H, d, J = 8.2 Hz), 7.44 (1H, dd, J = |
| | 8.1, 2.4 Hz), 7.56 (1H, d, J = 8.1 Hz), 7.75-7.94 (2H, m), 8.36 (1H, d, J = 2.3 Hz), 8.41 (1H, |
| | t, J = 5.9 Hz) 2H overlapping with water, peak not resolved. |
| 2029 | 1.10 (3H, t, J = 7.2 Hz), 2.03 (3H, s), 2.96 (2H, t, J = 6.7 Hz), 3.02 (3H, s), 3.22-3.29 (4H, |
| | m), 3.53 (2H, t, J = 6.7 Hz), 4.07 (2H, t, J = 6.7 Hz), 7.30 (1H, d, J = 8.2 Hz), 7.37-7.40 (1H, |
| | m), 7.42 (1H, dd, J = 8.1, 2.4 Hz), 7.48 (1H, dd, J = 8.2, 2.2 Hz), 7.52 (1H, d, J = 2.3 Hz), 7.78 |
| | (1H, d, J = 2.3 Hz), 8.41 (1H, t, J = 5.9 Hz) |
| 2030 | 1.11 (3H, t, J = 7.2 Hz), 2.98 (2H, t, J = 6.6 Hz), 3.03 (3H, s), 3.26 (4H, m), 3.54 (2H, t, J = |
| | 6.6 Hz), 4.08 (2H, t, J = 6.7 Hz), 7.32 (1H, d, J = 8.2 Hz), 7.44 (1H, dd, J = 8.1, 2.4 Hz), 7.49 |
| | (1H, t, J = 9.3 Hz), 7.65 (1H, m), 7.79 (1H, d, J = 2.4 Hz), 7.84 (1H, dd, J = 6.4, 2.8 Hz), 8.48 |
| | (1H, t, J = 5.9 Hz). 2H overlapping with water, peak not resolved |
| 2031 | 1.12 (3H, t, J = 7.1 Hz), 2.99 (2H, t, J = 6.7 Hz), 3.04 (3H s), 3.20 (2H, q, J = 7.4 Hz), 3.28 |
| | (2H, t, J = 6.7 Hz), 3.56 (2H, t, J = 6.6 Hz), 4.04 (2H, t, J = 6.6 Hz), 6.67 (2H, s), 7.32 (1H, d, |
| | J = 8.1 Hz), 7.45 (1H, d, J = 7.1 Hz), 7.82 (1H, d, J = 2.4 Hz), 7.92 (1H, s), 8.15 (1H, s), 8.40 |
| | (1H, t, J = 6.0 Hz). 2H overlapping with water, peak not resolved |
| 2032 | 1.11 (2H, t, J = 7.2 Hz), 1.29 (1H, t, J = 7.0 Hz), 2.24-2.31 (2H, m), 2.90-3.09 (6H, m), 3.22 |
| | -3.29 (2H, m), 3.37 (1H, t, J = 6.5 Hz), 3.49-3.63 (2H, m), 4.04-4.12 (2H, m), 4.27-4.37 |
| | (1H, m), 7.19-7.39 (2H, m), 7.40-7.45 (2H, m), 7.46-7.62 (1H, m), 7.77-7.91 (1H, m), |
| | 8.42 (1H, t, J = 5.9 Hz). Split peaks present |
| 2033 | 1.12 (3H, t, J = 7.2 Hz), 2.98 (2H, t, J = 6.6 Hz), 3.04 (3H, s), 3.22 (2H, t, J = 6.6 Hz), 3.26- |
| | 3.31 (2H, m), 3.56 (2H, t, J = 6.6 Hz), 4.07 (2H, t, J = 6.6 Hz), 4.56 (2H, br s), 5.47 (1H, s), |
| | 7.32 (1H, d, J = 8.1 H, ), 7.44-7.47 (2H, m), 7.55 (1H, t, J = 1.6 Hz), 7.65 (1H, t, J = 2.0 Hz), |
| | 7.83 (1H, d, J = 2.3 Hz), 8.44 (1H, t, J = 5.9 Hz) |
| 2034 | 1.10 (3H, t, J = 7.2 Hz), 2.38 (3H, s), 2.96 (2H, t, J = 6.7 Hz), 3.02 (3H, s), 3.20-3.30 (4H, |
| | m), 3.53 (2H, t, J = 6.7 Hz), 4.06 (2H, t, J = 6.7 Hz), 7.13 (1H, d, J = 7.3 Hz ), 7.22 (1H, d, J = |
| | 12.4 Hz), 7.30 (1H, d, J = 8.1 Hz), 7.42 (1H, dd, J = 8.1, 2.4 Hz), 7.48 (1H, t, J = 8.0 Hz), 7.78 |
| | (1H, d, J = 2.3 Hz), 8.41 (1H, t, J = 6.0 Hz) |
| 2035 | 1.11 (3H, t, J = 7.2 Hz), 2.40 (3H, s), 2.97 (2H, t, J = 6.7 Hz), 3.02 (3H, s), 3.22 (2H), 3.27 |
| | (2H, d, J = 6.7 Hz), 3.54 (2H, t, J = 6.6 Hz), 4.08 (2H, t, J = 6.5 Hz), 7.31 (1H, d, J = 8.1 Hz), |
| | 7.37 (1H, d, J = 5.1 Hz), 7.44 (1H, dd, J = 8.1, 2.4 Hz), 7.52 (1H, s), 7.80 (1H, d, J = 2.3 Hz), |
| | 8.37 (1H, d, J = 5.0 Hz), 8.41 (1H, t, J = 5.9 Hz). 2H overlapping with water, peak not |
| | resolved. |
| 2036 | 1.10 (3H, t, J = 7.2 Hz), 2.02 (3H, s), 2.96 (2H, t, J = 6.6 Hz), 3.02 (3H, s), 3.26 (4H, m), 3.53 |
| | (2H, t, J = 6.7 Hz), 4.08 (2H, t, J = 6.7 Hz), 7.29 (1H, d, J = 8.2 Hz), 7.34 (1H, td, J = 7.9, 0.7 |
| | Hz), 7.39 (1H, dd, J = 8.0, 1.5 Hz), 7.42 (1H, dd, J = 8.1, 2.4 Hz), 7.60 (1H, dd, J = 7.9, 1.4 |
| | Hz), 7.77 (1H, d, J = 2.3 Hz), 8.41 (1H, t, J = 5.9 Hz) |
| 2037 | 1.11 (3H, t, J = 7.1 Hz), 2.97 (2H, t, J = 6.7 Hz), 3.02 (3H, s), 3.23 (2H, t, J = 6.6 Hz), 3.28 |
| | (2H, t, J = 7.3, 6.1 Hz), 3.54 (2H, t, J = 6.6 Hz), 3.82 (3H, s), 4.08 (2H, t, J = 6.6 Hz), 6.96 (1H, |
| | d, J = 8.2 Hz), 7.25 (1H, d, J = 7.4 Hz), 7.31 (1H, d, J = 8.1 Hz), 7.44 (1H, dd, J = 8.1, 2.4 Hz), |
| | 7.81 (1H, d, J = 2.3 Hz), 7.88-7.93 (1H, m), 8.42 (1H, t, J = 5.9 Hz) |
| 2038 | 2.19 (3H, s), 2.96 (2H, t, J = 6.6 Hz), 3.02 (3H, s), 3.04 (2H, d, J = 6.7 Hz), 3.32 (3H, s), 3.53 |
| | (2H, t, J = 7.1, 6.2 Hz), 4.07 (2H, t, J = 6.8 Hz), 4.52 (2H, s), 7.29 (1H, d, J = 8.1 Hz), 7.41 |
| | (1H, dd, J = 8.1, 2.4 Hz), 7.49 (1H, t, J = 7.9), 7.67 (1H, dd, J = 8.0, 1.3 Hz), 7.77 (1H, d, J = |
| | 2.3 Hz), 7.91 (1H, dd, J = 7.8, 1.3 Hz) |
| 2039 | 1.91-2.02 (1H, m), 2.08-2.18 (1H, m), 2.97 (2H, t, J = 6.6 Hz), 3.02 (3H, s), 3.24 (2H, t, J = |
| | 6.7 Hz), 3.54 (2H, t, J = 6.7 Hz), 3.58 (1H, dd, J = 8.7, 4.9 Hz), 3.66-3.74 (1H, m), 3.78- |
| | 3.88 (2H, m), 4.09 (2H, t, J = 6.7 Hz), 4.41-4.52 (1H, m), 7.31 (1H, d, J = 8.2 Hz), 7.38 (1H, |
| | td, J = 8.2, 1.4 Hz), 7.43 (1H, dd, J = 8.1, 2.4 Hz), 7.67 (1H, ddd, J = 8.2, 6.7, 1.6 Hz), 7.74- |
| | 7.78 (1H, m), 7.79 (1H, d, J = 2.2 Hz), 8.57 (1H, d, J = 7.0 Hz) |
| 2040 | 2.16 (3H, s), 2.97 (2H, t, J = 6.6 Hz), 3.02 (3H, s), 3.25 (2H, t, J = 6.7 Hz), 3.54 (2H, t, J = 6.6 |
| | Hz), 4.09 (2H, t, J = 6.7 Hz), 4.35 (2H, d, J = 6.0 Hz), 5.91 (1H, s), 7.29-7.33 (1H, m), 7.38 |
| | (1H, td, J = 8.2, 1.5 Hz), 7.42-7.45 (1H, m), 7.66 (1H, ddd, J = 8.2, 6.7, 1.7 Hz), 7.73-7.80 |
| | (2H, m), 8.71 (1H, s), 12.21 (1H, s) |
| 2041 | 2.98 (4H, m ), 3.03 (3H, s), 3.24 (2H, d, J = 6.1 Hz), 3.32 (3H, s), 3.55 (2H, t, J = 6.6 Hz), |
| | 4.06 (2H, t, J = 6.5 Hz), 4.51 (2H, s), 7.31 (1H, d, J = 8.1 Hz), 7.45 (4H, m ), 7.82 (1H, s ) NH2 |
| | not observed |
| 2042 | 1.10 (3H, t, J = 7.1 Hz), 2.21 (3H, s), 2.96 (2H, t, J = 6.7 Hz), 3.02 (3H, s), 3.26 (4H, d, J = 7.0 |
| | Hz), 3.53 (2H, t, J = 6.7 Hz), 4.08 (2H, t, J = 6.7 Hz), 7.29 (1H, d, J = 8.2 Hz), 7.42 (1H, dd, J = |
| | 8.0, 2.4 Hz), 7.53 (1H, t, J = 7.9 Hz), 7.73-7.80 (2H, m), 7.96 (1H, d, J = 7.8 Hz), 8.42 (1H, |
| | s) |
| 2043 | 0.22-0.51 (4H, m), 0.96-1.09 (1H, m ), 2.97 (2H, t, J = 6.6 Hz), 3.02 (3H, s), 3.20-3.32 |
| | (2H, m), 3.35-3.46 (1H, m), 3.49 (4H, m ), 4.09 (2H, t, J = 6.7 Hz), 4.73 (1H, t, J = 5.5 Hz), |
| | 7.31 (1H, d, J = 8.2 Hz), 7.39 (1H, dd, J = 8.2, 1.5 Hz), 7.44 (1H, dd, J = 8.0, 2.4 Hz) , 7.68 |
| | (1H, ddd, J = 8.2, 6.7, 1.6 Hz), 7.73-7.82 (2H, m), 8.07 (1H, d, J = 9.0 Hz) |
| 2044 | 2.43 (3H, s), 2.97 (2H, t, J = 6.7 Hz), 3.02 (3H, s), 3.24 (2H, t, J = 6.7 Hz), 3.54 (2H, t, J = 6.7 |
| | Hz), 4.09 (2H, t, J = 6.7 Hz), 4.40 (2H, d, J = 6.2 Hz), 7.20 (1H, d, J = 8.0 Hz), 7.31 (1H, d, J = |
| | 8.2 Hz), 7.38 (1H, td, J = 8.2, 1.4 Hz), 7.43 (1H, dd, J = 8.1, 2.4 Hz), 7.62 (1H, dd, J = 7.9, J = |
| | 2.3 Hz), 7.64-7.69 (1H, m) , 7.73-7.83 (2H, m), 8.41 (1H, d, J = 2.5 Hz), 9.10 (1H, t, J = |
| | 6.3 Hz) |
| 2045 | 1.11 (3H, t, J = 7.2 Hz), 2.20 (3H, s), 2.98 (2H, t, J = 6.6 Hz), 3.03 (3H, s), 3.21 (2H, t, J = 6.6 |
| | Hz), 3.27 (2H, dd, J = 7.2, 5.9 Hz), 3.55 (2H, t, J = 6.7 Hz), 4.03 (2H, t, J = 6.7 Hz), 7.10 (1H, |
| | s), 7.26-7.37 (2H, m), 7.45 (1H, dd, J = 8.1, 2.4 Hz), 7.82 (1H, s), 8.39 (1H, t, J = 5.9 Hz) |
| 2046 | 0.18-0.27 (2H, m), 0.33-0.47 (2H, m), 0.96-1.12 (1H, m), 3.08 (3H, s), 3.11 (2H, t, J = |
| | 6.5 Hz), 3.24 (2H, t, J = 6.7 Hz), 3.54 (2H, t, J = 5.0 Hz), 4.07 (2H, t, J = 6.7 Hz), 4.36 (2H, t , |
| | J = 5.4 Hz), 7.02 (1H, d, J = 8.6 Hz), 7.35-7.41 (1H, m), 7.42 (1H, dd, J = 8.6, 2.8 Hz), 7.58 |
| | (1H, d, J = 2.8 Hz), 7.66 (1H, ddd, J = 8.2, 6.7, 1.6 Hz), 7.77 (1H, ddd, J = 8.3, 6.8, 1.6 Hz), |
| | 8.53 (1H, t, J = 6.0 Hz) |
| 2047 | 2.90-3.19 (7H, m), 3.57 (2H, t, J = 6.7 Hz), 3.86 (2H, s), 4.01 (2H, t, J = 6.4 Hz), 7.16-7.62 |
| | (3H, m), 7.62-7.99 (2H, m), 8.26 (1H, s), 8.56 (1H, s) NH₂ not observed |
| 2048 | 1.90-2.02 (1H, m), 2.05-2.19 (1H, m), 3.08 (3H, s), 3.23 (2H, t, J = 6.7 Hz), 3.54 (2H, t, J = |
| | 5.1 Hz), 3.57 (1H, dd, J = 8.8, 4.9 Hz), 3.65-3.76 (1H, m), 3.77-3.90 (2H, m), 4.07 (2H, t, |
| | J = 6.7 Hz), 4.36 (2H, dd, J = 5.5, 4.5 Hz), 4.41-4.54 (1H, m), 7.03 (1H, d, J = 8.7 Hz), 7.34- |
| | 7.45 (2H, m), 7.58 (1H, d, J = 2.7 Hz), 7.62-7.70 (1H, m), 7.73-7.82 (1H, m), 8.56 (1H, d, |
| | J = 7.0 Hz) |
| 2049 | 0.22-0.41 (3H, m), 0.41-0.50 (1H, m), 0.97-1.08 (1H, m), 3.08 (3H, s), 3.24 (2H, td, J = |
| | 6.7, 3.3 Hz), 3.35-3.43 (1H, m), 3.49-3.64 (4H, m), 4.07 (2H, t, J = 6.8 Hz), 4.36 (2H, dd, J = |
| | 5.5, 4.4 Hz), 4.73 (1H, t, J = 5.5 Hz), 7.03 (1H, d, J = 8.7 Hz), 7.36-7.41 (1H, m), 7.42 (1H, |
| | dd, J = 8.7, 2.8 Hz), 7.59 (1H, d, J = 2.7 Hz), 7.64-7.71 (1H, m), 7.73-7.82 (1H, m), 8.06 |
| | (1H, d, J = 8.9 Hz) |
| 2050 | 2.10 (6H, s), 3.08 (3H, s), 3.22 (2H, t, J = 6.7 Hz), 3.54 (2H, t, J = 5.1 Hz), 4.06 (2H, t, J = 6.7 |
| | Hz), 4.36 (2H, t, J = 4.5 Hz), 6.21 (1H, s), 7.02 (1H, d, J = 8.7 Hz), 7.38 (1H, td, J = 8.2, 1.5 |
| | Hz), 7.42 (1H, dd, J = 8.7, 2.8 Hz), 7.58 (1H, d, J = 2.8 Hz), 7.60-7.68 (1H, m), 7.71-7.82 |
| | (1H, m), 8.92 (1H, s) |
| 2051 | 1.08 (3H, t, J = 7.0 Hz), 2.33 (3H, s), 2.95 (2H, t, J = 6.7 Hz), 3.00 (3H, s), 3.18 (2H, t, J = 6.5 |
| | Hz), 3.24 (2H, q, J = 7.1 Hz), 3.52 (2H, t, J = 6.7 Hz), 4.03 (2H, t, J = 6.6 Hz), 7.29 (1H, d, J = |
| | 8.2 Hz), 7.34 (1H, d, J = 1.6 Hz), 7.39-7.46 (2H, m), 7.55 (1H, d, J = 2.0 Hz), 7.80 (1H, d, J = |
| | 2.4 Hz), 8.43 (1H, t, J = 6.0 Hz) |
| 2052 | 1.10 (3H, t, J = 7.2 Hz), 2.96 (2H, t, J = 6.6 Hz), 3.02 (3H, s), 3.22-3.30 (4H, m), 3.53 (2H, t, |
| | J = 6.7 Hz), 4.06 (2H, t, J = 6.8 Hz), 4.30 (2H, d, J = 5.7 Hz), 4.77 (1H, t, J = 5.7 Hz), 7.29 (1H, |
| | d, J = 8.2 Hz), 7.38-7.46 (3H, m), 7.58-7.65 (1H, m), 7.77 (1H, d, J = 2.4 Hz), 8.43 (1H, t, |
| | J = 5.9 Hz) |
| 2053 | 0.18-0.55 (3H, m), 0.96-1.09 (1H, m), 3.08 (3H, s), 3.20-3.28 (2H, m), 3.50-3.61 (4H, |
| | m), 4.07 (2H, t, J = 6.8 Hz), 4.36 (2H, dd, J = 5.5, 4.5 Hz), 4.73 (1H, t, J = 5.6 Hz), 7.03 (1H, |
| | d, J = 8.6 Hz), 7.34-7.46 (2H, m), 7.58 (1H, d, J = 2.7 Hz), 7.63-7.72 (1H, m), 7.73-7.82 |
| | (1H, m), 8.05 (1H, d, J = 9.0 Hz) One exchangeable proton not observed and 1H |
| | overlapping with water peak not resolved |
| 2054 | 1.11 (3H, t, J = 7.2 Hz), 2.97 (2H, t, J = 6.6 Hz), 3.03 (3H, s), 3.11-3.30 (4H, m), 3.54 (2H, t, |
| | J = 6.7 Hz), 4.04 (2H, t, J = 6.6 Hz), 6.99 (1H, d, J = 8.7 Hz), 7.31 (1H, d, J = 8.1 Hz), 7.37 |
| | (1H, dd, J = 8.7, 2.6 Hz), 7.43 (1H, dd, J = 8.1, 2.3 Hz), 7.62 (1H, d, J = 2.6 Hz), 7.81 (1H, d, |
| | J = 2.3 Hz), 8.38 (1H, t, J = 5.9 Hz), 10.70 (1H, s) |
| 2056 | 2.99 (4H, t, J = 6.7 Hz), 3.05 (4H, s), 3.26 (3H, t, J = 6.6 Hz), 3.56 (3H, t, J = 6.7 Hz), 4.07 |
| | (2H, t, J = 6.6 Hz), 7.29 (1H, d, J = 8.2 Hz), 7.44 (1H, dd, J = 8.1, 2.4 Hz), 7.49 (1H, s), 7.55 |
| | (2H, d, J = 6.8 Hz), 7.86 (1H, d, J = 2.3 Hz) Three exchangeable protons not observed |
| 2057 | 1.12 (3H, t, J = 7.1 Hz), 2.37 (3H, s), 2.98 (2H, t, J = 6.7 Hz), 3.03 (3H, s), 3.22 (2H, t, J = 6.7 |
| | Hz), 3.28 (2H, d, J = 6.9 Hz), 3.55 (2H, t, J = 6.6 Hz), 4.07 (2H, t, J = 6.6 Hz), 7.32 (1H, d, J = |
| | 8.2 Hz), 7.42-7.55 (3H, m), 7.64 (1H, d, J = 2.3 Hz), 7.82 (1H, d, J = 2.4 Hz), 8.41 (1H, s) |
| 2058 | 1.12 (3H, t, J = 7.3 Hz), 2.98 (2H, s), 3.03 (3H, s), 3.22 (7H, s), 3.55 (2H, s), 4.07 (2H, s), |
| | 4.47 (2H, s), 7.32 (1H, d, J = 8.1 Hz), 7.46 (2H, s), 7.57 (1H, s), 7.72 (1H, s), 7.83 (1H, s), |
| | 8.46 (1H, s). |
| 3001 | 2.35 (3H, s), 2.62 (2H, t, J = 5.9 Hz), 2.88 (2H, t, J = 5.9 Hz), 3.52 (2H, s), 7.18 (1H, d, J = 2.1 |
| | Hz), 7.21-7.33 (2H, m), 7.55-7.68 (2H, m), 7.72-7.79 (1H, m), 7.87-7.93 (1H, m), 8.49 |
| | (1H, s) |
| 3002 | 2.21 (3H, s), 2.35 (3H, s), 2.55-2.72 (2H, m), 2.89 (2H, s), 3.34-3.61 (2H, m), 7.09 (1H, d, |
| | J = 2.1 Hz), 7.15 (1H, dd, J = 8.0, 2.2 Hz), 7.30 (1H, d, J = 8.1 Hz), 7.54-7.66 (2H, m), 7.69- |
| | 7.76 (1H, m), 7.87 (1H, t, J = 1.9 Hz) |
| 3003 | 0.27-0.32 (2H, m), 0.46-0.52 (2H, m), 1.04-1.15 (1H, m), 2.23 (3H, s), 2.58 (2H, t, |
| | J = 6.2 Hz), 2.68-2.75 (1H, m), 2.76-2.83 (1H, m), 2.85-2.94 (2H, m), 3.28-3.31 (2H, |
| | m), 3.55-3.63 (3H, m), 3.68 (1H, d, J = 15.4 Hz), 4.50 (1H, t, J = 5.4 Hz), 7.09-7.13 (1H, m), |
| | 7.17 (1H, dd, J = 8.0, 2.2 Hz), 7.29 (1H, d, J = 8.1 Hz), 7.53-7.59 (2H, m), 7.69-7.76 (1H, |
| | m), 7.84-7.88 (1H, m), 8.68 (1H, t, J = 5.8 Hz) |
| 3004 | 0.27-0.32 (2H, m), 0.46-0.51 (2H, m), 1.04-1.15 (1H, m), 2.22 (3H, s), 2.35 (3H, s), |
| | 2.56-2.63 (1H, m), 2.64-2.71 (1H, m), 2.85-2.94 (2H, m), 3.27-3.31 (2H, m), 3.45 |
| | (1H, d, J = 15.3 Hz), 3.56 (1H, d, J = 15.3 Hz), 7.11 (1H, d, J = 2.1 Hz), 7.17 (1H, dd, J = 8.0, 2.2 |
| | Hz), 7.30 (1H, d, J = 8.1 Hz), 7.53-7.59 (2H, m), 7.68-7.77 (1H, m), 7.84-7.87 (1H, m), |
| | 8.68 (1H, t, J = 5.8 Hz). |
| 3005 | 2.00-2.05 (3H, m), 2.20 (3H, s), 2.57 (2H, t, J = 6.2 Hz), 2.66-2.83 (2H, m), 2.84-2.90 |
| | (2H, m), 3.53-3.66 (4H, m), 3.74 (3H, s), 4.47-4.54 (3H, m), 7.10 (1H, d, J = 2.1 Hz), 7.16 |
| | (1H, dd, J = 8.1, 2.2 Hz), 7.28 (1H, d, J = 8.2 Hz), 7.43 (1H, d, J = 0.9 Hz), 7.51-7.60 (2H, |
| | m), 7.68-7.77 (1H, m), 7.83-7.89 (1H, m), 8.91 (1H, t, J = 5.3 Hz) |
| 3006 | 2.00-2.06 (3H, m), 2.19 (3H, s), 2.35 (3H, s), 2.54-2.72 (2H, m), 2.85-2.91 (2H, m), 3.40- |
| | 3.59 (2H, m), 3.74 (3H, s), 4.51 (2H, d, J = 5.3 Hz), 7.11 (1H, d, J = 2.1 Hz), 7.17 (1H, dd, |
| | J = 8.0, 2.2 Hz), 7.30 (1H, d, J = 8.1 Hz), 7.41-7.46 (1H, m), 7.51-7.60 (2H, m), 7.68-7.78 |
| | (1H, m), 7.83-7.89 (1H, m), 8.90 (1H, t, J = 5.3 Hz) |
| 3007 | 0.85-0.99 (4H, m), 2.17 (3H, s), 2.54-2.83 (5H, m), 2.86-2.90 (2H, m), 3.54-3.73 (4H, |
| | m), 4.49-4.55 (3H, m), 7.08-7.12 (1H, m), 7.12-7.19 (1H, m), 7.28 (1H, d, J = 8.1 Hz), |
| | 7.46-7.57 (2H, m), 7.62-7.76 (2H, m), 7.79-7.85 (1H, m) |
| 3008 | 0.61-0.74 (4H, m), 1.60-1.71 (1H, m), 2.16 (3H, s), 2.57 (2H, t, J = 6.2 Hz), 2.66-2.89 |
| | (2H, m), 2.86-2.90 (2H, m), 3.54-3.70 (4H, m), 4.50 (1H, t, J = 5.4 Hz), 4.61 (2H, d, J = 5.4 |
| | Hz), 7.06-7.11 (1H, m), 7.11-7.18 (1H, m), 7.28 (1H, d, J = 8.3 Hz), 7.43-7.56 (2H, m), |
| | 7.63-7.72 (1H, m), 7.77-7.83 (1H, m), 8.63 (1H, t, J = 5.4 Hz) |
| 3009 | 2.17 (3H, s), 2.18-2.21 (3H, m), 2.57 (2H, t, J = 6.2 Hz), 2.65-2.84 (2H, m), 2.84-2.90 |
| | (2H, m), 3.54-3.72 (4H, m), 3.82 (3H, s), 4.50 (1H, t, J = 5.4 Hz), 4.76 (2H, d, J = 5.8 Hz), |
| | 7.07-7.12 (1H, m), 7.12-7.18 (1H, m), 7.28 (1H, d, J = 8.1 Hz), 7.42-7.54 (2H, m), 7.54- |
| | 7.59 (1H, m), 7.62-7.69 (1H, m), 7.74-7.80 (1H, m), 8.31 (1H, t, J = 5.7 Hz) |
| 3010 | 2.17 (3H, s), 2.42 (3H, s), 2.55-2.64 (2H, m), 2.69-2.83 (2H, m), 2.86-2.91 (2H, m), 3.55- |
| | 3.63 (2H, m), 3.61-3.72 (2H, m), 4.50 (1H, s), 4.81 (2H, d, J = 5.6 Hz), 7.09-7.19 (2H, |
| | m), 7.25-7.34 (2H, m), 7.43-7.56 (2H, m), 7.63-7.73 (1H, m), 7.77-7.83 (1H, m), 8.47 |
| | (1H, d, J = 5.1 Hz), 8.57-8.62 (1H, m), 9.12 (1H, t, J = 5.6 Hz) |
| 3011 | 2.19 (3H, s), 2.41 (3H, s), 2.52-2.61 (2H, m), 2.65-2.83 (2H, m), 2.84-2.90 (2H, m), 3.47- |
| | 3.71 (4H, m), 4.50 (1H, t, J = 5.4 Hz), 4.64 (2H, d, J = 5.9 Hz), 7.07-7.19 (2H, m), 7.21- |
| | 7.31 (2H, m), 7.51-7.60 (2H, m), 7.68-7.77 (1H, m), 7.83-7.89 (1H, m), 8.36 (1H, d, J = |
| | 4.9 Hz), 8.49 (1H, s), 9.03 (1H, t, J = 6.0 Hz) |
| 3012 | 2.21 (3H, s), 2.57 (2H, t, J = 6.1 Hz), 2.67-2.84 (2H, m), 2.85-2.90 (2H, m), 3.55-3.72 |
| | (4H, m), 4.50 (1H, t, J = 5.4 Hz), 7.08 (1H, d, J = 2.1 Hz), 7.11-7.17 (1H, m), 7.29 (1H, d, J = |
| | 8.1 Hz), 7.53-7.62 (1H, m), 7.59-7.66 (1H, m), 7.68-7.76 (1H, m), 7.84-7.90 (1H, m) |
| 3013 | 2.52-2.61 (2H, m), 2.74 (2H, t, J = 5.8 Hz), 2.87 (2H, t, J = 5.8 Hz), 3.59 (2H, q, J = 6.0 Hz), |
| | 3.64 (2H, s), 4.50 (1H, t, J = 5.4 Hz), 7.18 (1H, d, J = 2.2 Hz), 7.21-7.32 (2H, m), 7.55-7.65 |
| | (1H, m), 7.61-7.68 (1H, m), 7.71-7.79 (1H, m), 7.88-7.93 (1H, m), 8.49 (1H, s) |
| 3014 | 0.26-0.34 (2H, m), 0.43-0.55 (2H, m), 1.03-1.18 (1H, m), 2.21 (3H, s), 3.05 (3H, s), 3.08 |
| | (2H, t, J = 6.7 Hz), 3.22-3.37 (2H, m), 3.56-3.67 (2H, m), 7.47-7.62 (4H, m), 7.69-7.79 |
| | (1H, m), 7.84-7.94 (2H, m), 8.68 (1H, t, J = 5.8 Hz) |
| 3015 | 2.19 (3H, s), 2.21 (3H, d, J = 0.7 Hz), 3.05 (2H, t, J = 6.6 Hz), 3.52-3.61 (4H, m), 3.64-3.72 |
| | (5H, m), 4.48 (2H, d, J = 5.7 Hz), 4.79 (1H, br s), 6.00 (1H, d, J = 0.8 Hz), 7.47-7.60 (4H, |
| | m), 7.69-7.79 (1H, m), 7.85-7.87 (1H, m), 7.90-7.92 (1H, m), 8.94 (1H, t, J = 5.6 Hz) |
| 3016 | 0.26-0.34 (2H, m), 0.43-0.55 (2H, m), 1.04-1.17 (1H, m), 2.21 (3H, s), 3.06 (2H, t, J = |
| | 6.6 Hz), 3.23-3.31 (2H, m), 3.47-3.64 (4H, m), 3.68 (2H, t, J = 6.6 Hz), 4.80 (1H, br s), |
| | 7.47-7.60 (4H, m), 7.67-7.79 (1H, m), 7.85-7.89 (1H, m), 7.92 (1H, d, J = 2.0 Hz), 8.68 |
| | (1H, t, J = 5.8 Hz) |
| 3017 | 2.17 (3H, s), 2.41 (3H, d, J = 0.7 Hz), 3.05 (2H, t, J = 6.6 Hz), 3.52-3.61 (4H, m), 3.67 (2H, t, |
| | J = 6.6 Hz), 4.62-4.72 (2H, m), 4.77-4.81 (1H, m), 7.23 (1H, dt, J = 5.0, 0.7 Hz), 7.46- |
| | 7.55 (2H, m), 7.51-7.60 (2H, m), 7.69-7.79 (1H, m), 7.87-7.89 (1H, m), 7.89-7.93 (1H, |
| | m), 8.36 (1H, d, J = 4.9 Hz), 8.50 (1H, s), 9.02 (1H, t, J = 5.9 Hz) |
| 3018 | 2.16 (3H, s), 3.01-3.09 (2H, m), 3.49-3.63 (4H, m), 3.67 (2H, t, J = 6.6 Hz), 4.76-4.84 |
| | (1H, m), 7.46-7.60 (4H, m), 7.65-7.74 (1H, m), 7.80-7.86 (1H, m), 7.86-7.91 (1H, m), |
| | 13.41 (1H, s) |
| 3019 | 1.36 (3H, t, J = 7.1 Hz), 2.17 (3H, s), 3.01-3.09 (2H, m), 3.49-3.61 (4H, m), 3.67 (2H, t, J = |
| | 6.6 Hz), 4.36-4.48 (2H, m), 4.80 (1H, t, J = 5.1 Hz), 7.46-7.61 (4H, m), 7.65-7.75 (1H, |
| | m), 7.81-7.86 (1H, m), 7.88-7.91 (1H, m) |
| 3020 | 2.13-2.21 (6H, m), 3.05 (3H, s), 3.07-3.11 (2H, m), 3.61 (2H, t, J = 6.6 Hz), 4.52 (2H, d, J = |
| | 5.6 Hz), 5.97 (1H, s), 7.49-7.58 (4H, m), 7.71-7.78 (1H, m), 7.87-7.89 (1H, m), 7.90- |
| | 7.92 (1H, m), 8.86-8.99 (1H, m), 12.29 (1H, s) |
| 3021 | 2.15-2.19 (3H, m), 2.20-2.22 (3H, m), 3.05 (3H, s), 3.07-3.10 (2H, m), 3.61 (2H, t, J = |
| | 6.5 Hz), 3.67 (3H, s), 4.48 (2H, d, J = 5.6 Hz), 6.00 (1H, s), 7.47-7.60 (4H, m), 7.70-7.77 |
| | (1H, m), 7.87-7.89 (1H, m), 7.91 (1H, d, J = 2.1 Hz), 8.94 (1H, t, J = 5.7 Hz) |
| 3022 | 1.19 (3H, t, J = 7.2 Hz), 2.20 (3H, s), 3.05 (3H, s), 3.08 (2H, t, J = 6.7 Hz), 3.36-3.48 (2H, |
| | m), 3.57-3.65 (2H, m), 7.47-7.61 (4H, m), 7.69-7.79 (1H, m), 7.86-7.88 (1H, m), 7.91 |
| | (1H, d, J = 2.0 Hz), 8.58 (1H, t, J = 5.8 Hz) |
| 3023 | 0.24-0.34 (2H, m), 0.44-0.54 (2H, m), 1.04-1.15 (1H, m), 2.25 (3H, s), 2.89 (3H, s), 3.27- |
| | 3.30 (2H, m), 3.43-3.60 (2H, m), 4.38-4.49 (2H, m), 7.05 (1H, d, J = 8.9 Hz), 7.43 (1H, |
| | td, J = 8.2, 1.5 Hz), 7.51 (1H, dd, J = 8.8, 2.6 Hz), 7.58 (1H, d, J = 2.6 Hz), 7.69 (1H, ddd, J = |
| | 8.3, 6.7, 1.6 Hz), 7.81 (1H, ddd, J = 8.3, 6.8, 1.6 Hz), 8.61 (1H, t, J = 5.8 Hz) |
| 4001 | 2.20 (3H, s), 2.57 (2H, t, J = 6.2 Hz), 2.67-2.75 (2H, m), 2.77-2.83 (2H, m), 3.58-3.64 |
| | (4H, m), 3.66 (3H, s), 4.36 (2H, d, J = 6.1 Hz), 4.46-4.54 (1H, m), 5.01 (2H, s), 5.96 (1H, s), |
| | 7.16 (1H, d, J = 8.4 Hz), 7.46-7.55 (2H, m), 7.56-7.66 (2H, m), 8.40-8.43 (1H, m), 8.53 |
| | (1H, t, J = 2.1 Hz), 9.06 (1H, t, J = 6.1 Hz) |
| 4002 | 0.15-0.22 (2H, m), 0.40-0.48 (2H, m), 0.88-0.99 (1H, m), 2.54-2.62 (2H, m), 2.69- |
| | 2.76 (2H, m), 2.77-2.85 (2H, m), 3.00 (2H, dd, J = 6.9, 5.5 Hz), 3.11 (2H, t, J = 6.7 Hz), |
| | 3.55-3.63 (4H, m), 3.97 (2H, t, J = 6.7 Hz), 4.46-4.53 (1H, m), 4.93 (2H, s), 7.04 (1H, s), 7.08- |
| | 7.13 (2H, m), 7.38-7.43 (2H, m), 7.99-8.06 (1H, m), 8.21-8.25 (1H, m), 8.42 (1H, t, J = |
| | 5.6 Hz) |
| 4003 | 2.39 (3H, s), 2.56 (2H, t, J = 6.3 Hz), 2.68-2.74 (2H, m), 2.76-2.82 (2H, m), 3.51 (2H, t, J = |
| | 6.7 Hz), 3.56-3.62 (4H, m), 3.99 (2H, t, J = 6.7 Hz), 4.47 (1H, s br), 4.53 (2H, d, J = 6.0 Hz), |
| | 6.95-7.06 (1H, m), 7.07-7.16 (2H, m), 7.22 (1H, d, J = 4.9 Hz), 7.38-7.61 (2H, m), 8.18 |
| | (1H, d, J = 7.2 Hz), 8.26-8.40 (2H, m), 8.43-8.56 (1H, m), 9.30 (1H, t, J = 6.0 Hz) |
| 4004 | 0.42-0.49 (2H, m), 0.62-0.69 (2H, m), 2.56 (2H, t, J = 6.2 Hz), 2.65-2.74 (3H, m), 2.77- |
| | 2.82 (2H, m), 3.10 (2H, t, J = 6.7 Hz), 3.54-3.64 (4H, m), 3.97 (2H, t, J = 6.7 Hz), 4.47 (1H, |
| | t, J = 5.4 Hz), 4.86 (2H, s), 7.00-7.05 (1H, m), 7.07-7.14 (2H, m), 7.38-7.43 (2H, m), |
| | 8.00-8.06 (1H, m), 8.20-8.24 (1H, m), 8.44 (1H, d, J = 4.2 Hz) |

### Biological Methods

Determination of Human Factor XIIa (hFXIIa) inhibition

In vitro inhibition of hFXIIa was determined using an IC₅₀ assay based on standard literature methods (see e.g Duckworth et al., Clinical and Experimental Allergy 52 (2022); Baeriswyl et al., ACS Chem. Biol., 2015, 10 (8) 1861; Bouckaert et al., European Journal of Medicinal Chemistry 110 (2016) 181). hFXIIa (Enzyme Research Laboratories) was incubated at 25° C. with the fluorogenic substrate H-DPro-Phe-Arg-AFC (Peptide Protein Science) and various concentrations of the test compound. Protease activity was measured by monitoring the fluorescence emitted from cleaved substrate over 5 min at 25° C. The linear rate of fluorescence increase per minute was expressed as percentage (%) activity. The Km for the cleavage of the substrate by hFXIIa was determined by standard transformation of the Michaelis-Menten equation, with subsequent inhibition assays performed at substrate Km concentration. IC₅₀ values were calculated as the concentration of inhibitor giving 50% inhibition of the uninhibited enzyme activity. Data acquired from this assay are shown in Table 7 below using the following scale:

| Category | IC₅₀ (nM) |
|---|---|
| AA | 1-10 |
| A | 10-100 |
| B | 100-300 |
| C | 300-1,000 |
| D | 1,000-3,000 |
| E | 3,000-10,000 |
| F | 10,000-40,000 |
| G0 | >4,000 |

TABLE 7
Human FXIIa data, molecular weight and LCMS data

| Example | Human FXIIa | Average | LCMS |
|---|---|---|---|
| No | IC50 (nM) | Molecular weight | Molecular Ion |
| 1001 | B | 554.1 | 554.3 |
| 1002 | C | 518.1 | 518.2 |
| 1003 | C | 555.1 | 555.2 |
| 1004 | D | 568.1 | 568.2 |
| 1005 | A | 526.0 | 526.2 |
| 1006 | C | 555.1 | 555.2 |
| 1007 | B | 493.0 | 493.2 |
| 1008 | C | 584.1 | 584.3 |
| 1009 | E | 518.1 | 518.2 |
| 1010 | C | 546.1 | 546.3 |
| 1011 | E | 492.0 | 492.2 |
| 1012 | C | 490.0 | 490.2 |
| 1013 | B | 464.9 | 465.1 |
| 1014 | C | 464.0 | 464.2 |
| 1015 | B | 527.0 | 527.3 |
| 1016 | F | 492.0 | 492.2 |
| 1017 | C | 540.1 | 540.1 |
| 1018 | D | 556.1 | 556.1 |
| 1019 | B | 566.1 | 566.2 |
| 1020 | C | 594.2 | 594.3 |
| 1021 | A | 540.1 | 540.2 |
| 1022 | A | 554.1 | 554.2 |
| 1023 | B | 532.1 | 532.3 |
| 1024 | B | 556.1 | 556.2 |
| 1025 | C | 520.0 | 520.2 |
| 1026 | C | 520.0 | 520.2 |
| 1027 | C | 534.1 | 534.2 |
| 1028 | C | 512.0 | 512.1 |
| 1029 | C | 552.1 | 552.1 |
| 1030 | B | 540.1 | 540.1 |
| 1031 | B | 504.0 | 504.1 |
| 1032 | C | 532.0 | 532.1 |
| 1033 | D | 533.0 | 533.1 |
| 1034 | C | 491.0 | 491.1 |
| 1035 | B | 518.1 | 518.2 |
| 1036 | C | 530.1 | 530.2 |
| 1037 | A | 534.1 | 534.2 |
| 1038 | C | 554.1 | 554.2 |
| 1039 | C | 554.1 | 554.1 |
| 1040 | D | 491.0 | 491.2 |
| 1041 | C | 492.0 | 492.2 |
| 1042 | C | 506.0 | 506.2 |
| 1043 | C | 520.0 | 520.2 |
| 1044 | B | 504.0 | 504.1 |
| 1045 | C | 506.0 | 506.2 |
| 1046 | C | 576.1 | 576.2 |
| 1047 | C | 506.0 | 506.2 |
| 1048 | C | 504.0 | 504.1 |
| 1049 | B | 534.1 | 534.3 |
| 1050 | C | 562.1 | 562.3 |
| 1051 | A | 570.1 | 570.3 |
| 1052 | B | 518.1 | 518.2 |
| 1053 | B | 548.1 | 548.2 |
| 1054 | B | 518.1 | 518.2 |
| 1055 | A | 520.0 | 520.2 |
| 1056 | C | 506.0 | 506.1 |
| 1057 | C | 533.0 | 533.2 |
| 1058 | C | 520.0 | 520.2 |
| 1059 | B | 532.1 | 532.3 |
| 1060 | B | 548.1 | 548.3 |
| 1061 | B | 548.1 | 548.4 |
| 1063 | B | 556.1 | 556.2 |
| 1064 | C | 534.1 | 534.3 |
| 1065 | A | 530.0 | 530.3 |
| 1066 | D | 436.9 | 437.1 |
| 1067 | D | 506.0 | 506.2 |
| 1068 | B | 516.0 | 516.2 |
| 1069 | C | 467.0 | 467.1 |
| 1070 | B | 534.1 | 534.2 |
| 1071 | D | 558.0 | 558.2 |
| 1072 | D | 504.0 | 504.1 |
| 1073 | D | 516.0 | 516.2 |
| 1074 | D | 476.0 | 476.2 |
| 1075 | B | 532.0 | 532.2 |
| 1076 | B | 550.1 | 550.2 |
| 1077 | A | 560.1 | 560.2 |
| 1078 | A | 557.0 | 557.2 |
| 1079 | A | 522.0 | 522.3 |
| 1080 | B | 536.0 | 536.4 |
| 1081 | C | 561.1 | 561.4 |
| 1082 | C | 532.1 | 532.4 |
| 1083 | B | 597.1 | 597.4 |
| 1084 | B | 576.1 | 576.5 |
| 1085 | B | 534.1 | 534.2 |
| 1086 | B | 544.1 | 544.2 |
| 1087 | B | 534.1 | 534.4 |
| 1088 | B | 560.1 | 560.2 |
| 1089 | A | 546.0 | 546.1 |
| 1090 | B | 520.0 | 520.2 |
| 1091 | B | 520.0 | 520.2 |
| 1092 | B | 548.1 | 548.2 |
| 1093 | B | 532.0 | 532.2 |
| 1094 | D | 548.0 | 548.3 |
| 1095 | D | 534.0 | 534.3 |
| 1096 | C | 548.1 | 548.2 |
| 1097 | D | 566.0 | 566.4 |
| 1098 | B | 519.0 | 519.3 |
| 1099 | A | 530.1 | 530.3 |
| 1100 | D | 515.0 | 515.2 |
| 1101 | C | 534.1 | 534.3 |
| 1102 | C | 558.1 | 558.4 |
| 1103 | B | 530.1 | 530.2 |
| 1104 | C | 556.1 | 556.3 |
| 1105 | B | 546.0 | 546.2 |
| 1106 | B | 550.1 | 550.2 |
| 1107 | A | 556.1 | 556.2 |
| 1108 | B | 536.0 | 536.2 |
| 1109 | A | 546.0 | 546.2 |
| 1110 | A | 571.1 | 571.3 |
| 1111 | A | 571.1 | 571.2 |
| 1112 | B | 564.1 | 564.2 |
| 1113 | C | 534.1 | 534.3 |
| 1114 | E | 534.0 | 534.3 |
| 1115 | C | 546.1 | 546.3 |
| 1116 | C | 560.1 | 560.3 |
| 1117 | B | 505.0 | 505.3 |
| 1118 | B | 521.0 | 521.3 |
| 1119 | C | 560.1 | 560.2 |
| 1120 | C | 573.1 | 573.3 |
| 1121 | B | 522.0 | 522.3 |
| 1122 | B | 538.0 | 538.3 |
| 1123 | B | 538.0 | 538.4 |
| 1124 | C | 573.1 | 573.5 |
| 1125 | A | 578.0 | 578.4 |
| 1126 | A | 571.1 | 571.2 |
| 1127 | A | 586.1 | 586.3 |
| 1128 | A | 585.1 | 585.3 |
| 1129 | A | 541.0 | 541.2 |
| 1130 | A | 575.0 | 575.2 |
| 1131 | B | 571.1 | 571.2 |
| 1132 | A | 560.1 | 560.2 |
| 1133 | B | 560.1 | 560.3 |
| 1134 | A | 589.1 | 589.3 |
| 1135 | A | 557.0 | 557.2 |
| 1136 | A | 571.1 | 571.2 |
| 1137 | A | 575.0 | 575.2 |
| 1138 | A | 587.1 | 587.3 |
| 1139 | A | 571.1 | 571.2 |
| 1140 | A | 555.1 | 555.3 |
| 1141 | A | 587.1 | 587.3 |
| 1142 | B | 556.0 | 556.2 |
| 1143 | B | 546.1 | 546.4 |
| 1144 | A | 597.1 | 597.3 |
| 1145 | B | 571.1 | 571.2 |
| 1146 | A | 586.1 | 586.2 |
| 1147 | A | 571.1 | 571.2 |
| 1148 | B | 585.1 | 585.3 |
| 1149 | C | 504.0 | 504.1 |
| 1150 | A | 570.1 | 570.3 |
| 1151 | B | 547.0 | 547.2 |
| 1152 | C | 534.1 | 534.2 |
| 1153 | C | 518.1 | 518.2 |
| 1154 | A | 591.5 | 591.1 |
| 1155 | A | 594.5 | 594.2 |
| 1156 | B | 590.1 | 590.3 |
| 1157 | A | 574.1 | 570.3 |
| 1158 | A | 569.1 | 569.4 |
| 1159 | A | 555.0 | 555.3 |
| 1160 | A | 587.1 | 585.4 |
| 1161 | A | 575.0 | 575.3 |
| 1162 | A | 625.0 | 625.2 |
| 1163 | A | 571.1 | 571.4 |
| 1164 | C | 562.1 | 562.2 |
| 1166 | A | 574.1 | 574.3 |
| 1167 | B | 547.0 | 547.2 |
| 1168 | A | 558.1 | 558.3 |
| 1169 | B | 548.1 | 548.3 |
| 1170 | A | 591.5 | 591.2 |
| 1171 | A | 571.1 | 571.2 |
| 1172 | A | 587.1 | 587.2 |
| 1173 | B | 585.1 | 585.2 |
| 1174 | B | 589.5 | 589.2 |
| 1175 | B | 522.0 | 522.3 |
| 1176 | C | 582.1 | 582.3 |
| 1177 | A | 569.1 | 569.2 |
| 1178 | B | 572.1 | 572.3 |
| 1179 | C | 536.0 | 536.2 |
| 1180 | C | 564.0 | 564.2 |
| 1181 | C | 562.0 | 562.4 |
| 1182 | E | 450.9 | 450.0 |
| 1183 | D | 436.9 | 436.0 |
| 1184 | D | 450.9 | 450.0 |
| 1185 | C | 559.0 | 559.2 |
| 1186 | B | 574.0 | 574.2 |
| 1187 | C | 571.0 | 571.5 |
| 1188 | B | 555.0 | 555.3 |
| 1189 | A | 569.1 | 569.3 |
| 1190 | F | 504.0 | 504.2 |
| 1191 | C | 550.1 | 550.2 |
| 1192 | E | 490.0 | 490.2 |
| 1193 | B | 561.1 | 561.2 |
| 1194 | C | 545.0 | 545.5 |
| 1195 | E | 492.0 | 492.1 |
| 1196 | E | 466.9 | 467.1 |
| 1197 | C | 548.0 | 548.3 |
| 1198 | C | 534.0 | 534.2 |
| 1199 | B | 558.0 | 558.2 |
| 1200 | B | 544.0 | 544.3 |
| 1201 | B | 555.0 | 555.3 |
| 1202 | C | 542.0 | 542.2 |
| 1203 | B | 534.0 | 534.2 |
| 1204 | B | 558.0 | 558.2 |
| 1205 | F | 558.0 | 558.3 |
| 1206 | D | 544.0 | 544.2 |
| 1207 | E | 504.0 | 504.2 |
| 1208 | D | 558.0 | 558.3 |
| 1209 | B | 583.1 | 583.4 |
| 1210 | B | 586.1 | 586.3 |
| 1211 | C | 532.0 | 532.3 |
| 1212 | D | 553.0 | 553.2 |
| 1213 | B | 571.0 | 571.4 |
| 1214 | C | 555.0 | 555.4 |
| 1215 | B | 555.0 | 555.4 |
| 1216 | B | 534.0 | 534.2 |
| 1217 | A | 588.1 | 588.2 |
| 1218 | B | 558.0 | 558.3 |
| 1219 | A | 572.1 | 572.4 |
| 1220 | A | 575.1 | 575.2 |
| 1221 | A | 588.1 | 588.3 |
| 1222 | A | 588.1 | 588.3 |
| 1223 | B | 547.0 | 547.1 |
| 1224 | B | 572.1 | 572.2 |
| 1225 | B | 558.0 | 558.2 |
| 1226 | B | 550.1 | 550.2 |
| 1227 | B | 576.1 | 576.2 |
| 1229 | B | 589.0 | 589.3 |
| 1230 | A | 587.1 | 587.2 |
| 1231 | B | 570.0 | 570.3 |
| 1232 | D | 464.9 | 465.2 |
| 1233 | B | 571.0 | 571.3 |
| 1234 | A | 588.1 | 588.5 |
| 1235 | B | 571.0 | 571.2 |
| 1236 | A | 585.1 | 585.3 |
| 1237 | B | 584.1 | 584.3 |
| 1238 | B | 572.1 | 572.3 |
| 1239 | B | 518.0 | 518.2 |
| 1240 | C | 541.0 | 541.1 |
| 1241 | D | 572.1 | 572.3 |
| 1242 | E | 518.0 | 518.3 |
| 1243 | D | 522.0 | 522.2 |
| 1244 | E | 506.0 | 506.1 |
| 1245 | B | 530.0 | 530.4 |
| 1246 | C | 550.0 | 550.2 |
| 1247 | B | 545.0 | 545.5 |
| 1248 | B | 544.0 | 544.6 |
| 1249 | C | 550.0 | 550.2 |
| 1250 | C | 534.0 | 534.2 |
| 1251 | C | 550.0 | 550.2 |
| 1252 | C | 550.0 | 550.1 |
| 1253 | B | 536.0 | 536.1 |
| 1254 | A | 590.1 | 590.3 |
| 1255 | B | 574.0 | 574.3 |
| 1256 | A | 599.1 | 599.5 |
| 1257 | C | 574.1 | 574.2 |
| 1258 | B | 628.1 | 628.3 |
| 1259 | B | 600.1 | 600.3 |
| 1260 | C | 564.0 | 564.2 |
| 1261 | C | 548.0 | 548.2 |
| 1262 | B | 602.1 | 602.2 |
| 1263 | F | 518.0 | 518.1 |
| 1264 | C | 558.0 | 558.6 |
| 1265 | C | 564.0 | 564.2 |
| 1266 | C | 548.0 | 548.2 |
| 1267 | C | 556.0 | 556.2 |
| 1268 | C | 558.0 | 558.3 |
| 1269 | F | 493.0 | 493.1 |
| 1270 | F | 518.0 | 518.1 |
| 1271 | B | 571.0 | 571.2 |
| 1272 | E | 520.0 | 520.1 |
| 1273 | E | 493.0 | 493.1 |
| 1274 | C | 534.0 | 534.2 |
| 1275 | A | 602.1 | 602.2 |
| 1276 | E | 572.1 | 572.4 |
| 1277 | F | 518.0 | 518.2 |
| 1278 | E | 493.0 | 493.1 |
| 1279 | F | 493.0 | 493.1 |
| 1280 | C | 576.0 | 576.2 |
| 1281 | E | 510.0 | 510.1 |
| 1282 | C | 556.0 | 556.2 |
| 1283 | C | 590.1 | 590.2 |
| 1284 | C | 478.9 | 478.0 |
| 1285 | D | 518.0 | 518.2 |
| 1286 | F | 520.0 | 520.2 |
| 1287 | C | 569.1 | 569.6 |
| 1288 | A | 604.1 | 603.8 |
| 1289 | B | 550.0 | 549.9 |
| 1290 | B | 600.1 | 600.2 |
| 1292 | C | 574.1 | 574.3 |
| 1293 | B | 628.1 | 628.5 |
| 1294 | B | 548.0 | 547.9 |
| 1295 | F | 498.0 | 498.0 |
| 1296 | B | 572.1 | 572.2 |
| 1297 | C | 477.9 | 478.1 |
| 1298 | E | 494.0 | 494.1 |
| 1299 | A | 601.1 | 601.2 |
| 1300 | E | 556.0 | 556.3 |
| 1301 | D | 505.0 | 505.3 |
| 1302 | C | 556.0 | 556.3 |
| 1303 | C | 559.0 | 559.4 |
| 1304 | C | 558.0 | 558.4 |
| 1305 | A | 602.1 | 602.4 |
| 1306 | A | 599.1 | 599.4 |
| 1307 | B | 570.1 | 570.2 |
| 1308 | C | 555.0 | 555.2 |
| 1309 | B | 555.0 | 555.3 |
| 1310 | C | 556.0 | 556.4 |
| 1311 | E | 536.0 | 536.2 |
| 1312 | E | 522.0 | 522.1 |
| 1313 | E | 520.0 | 520.1 |
| 1314 | B | 569.1 | 569.4 |
| 1316 | F | 520.0 | 520.1 |
| 1317 | A | 588.1 | 588.2 |
| 1318 | A | 585.1 | 585.4 |
| 1319 | A | 574.0 | 574.2 |
| 1320 | B | 602.1 | 602.2 |
| 1321 | A | 587.0 | 587.4 |
| 1322 | F | 497.0 | 497.1 |
| 1323 | D | 478.9 | 479.1 |
| 1324 | D | 497.0 | 497.1 |
| 1325 | B | 599.0 | 599.3 |
| 1326 | B | 599.1 | 599.3 |
| 1327 | B | 599.1 | 599.4 |
| 1328 | C | 585.0 | 585.4 |
| 1329 | C | 618.1 | 618.4 |
| 1330 | D | 548.0 | 548.2 |
| 1331 | C | 602.1 | 602.3 |
| 1332 | C | 588.0 | 588.4 |
| 1333 | C | 547.0 | 547.2 |
| 1334 | C | 463.9 | 464.1 |
| 1335 | D | 490.0 | 490.1 |
| 1336 | C | 520.0 | 519.98 |
| 1337 | C | 508.0 | 508.5 |
| 1338 | C | 520.0 | 520.5 |
| 1339 | C | 520.0 | 520.5 |
| 1340 | C | 494.0 | 493.99 |
| 1341 | C | 507.9 | 507.94 |
| 1342 | C | 506.0 | 506.4 |
| 1343 | D | 506.0 | 506.0 |
| 1344 | C | 508.0 | 507.8 |
| 1345 | E | 528.0 | 527.8 |
| 1346 | C | 504.0 | 504.5 |
| 1347 | C | 465.9 | 466.5 |
| 1350 | E | 488.9 | 489.1 |
| 1351 | E | 604.1 | 604.3 |
| 1352 | A | 574.0 | 574.3 |
| 1353 | C | 475.9 | 478.5 |
| 1354 | D | 507.0 | 507.2 |
| 1355 | B | 600.1 | 600.2 |
| 1356 | D | 542.0 | 542.2 |
| 1357 | C | 585.1 | 585.3 |
| 1358 | D | 494.9 | 495.2 |
| 1359 | C | 504.0 | 504.5 |
| 1360 | C | 520.0 | 520.5 |
| 1361 | D | 534.0 | 534.2 |
| 1362 | E | 479.9 | 480.1 |
| 1363 | E | 494.0 | 494.1 |
| 1364 | D | 493.0 | 493.2 |
| 1365 | D | 478.0 | 478.1 |
| 1366 | E | 514.0 | 514.1 |
| 1367 | C | 490.0 | 490.1 |
| 1368 | D | 490.0 | 490.1 |
| 1369 | E | 519.0 | 519.2 |
| 1370 | C | 587.0 | 587.4 |
| 1371 | B | 508.0 | 508.2 |
| 1372 | C | 601.1 | 601.3 |
| 1373 | C | 604.1 | 604.33 |
| 1374 | B | 534.0 | 534.3 |
| 1375 | E | 521.0 | 521.3 |
| 1376 | A | 615.1 | 615.3 |
| 1377 | A | 618.1 | 618.3 |
| 1378 | A | 628.0 | 628.2 |
| 1379 | C | 618.1 | 618.3 |
| 1380 | C | 604.1 | 604.3 |
| 1381 | D | 538.0 | 538.3 |
| 1382 | A | 610.0 | 610.3 |
| 1383 | A | 725.2 | 725.4 |
| 1384 | C | 495.9 | 496.5 |
| 1385 | D | 478.0 | 477.9 |
| 1386 | D | 522.0 | 522.5 |
| 1387 | D | 522.0 | 522.9 |
| 1388 | C | 431.9 | 432.9 |
| 1389 | F | 504.0 | 504.1 |
| 1390 | D | 507.0 | 507.2 |
| 1391 | C | 522.0 | 521.9 |
| 1392 | E | 538.0 | 538.1 |
| 1393 | A | 617.1 | 617.2 |
| 1394 | A | 651.5 | 651.2 |
| 1395 | D | 604.1 | 604.2 |
| 1396 | C | 601.1 | 601.2 |
| 1397 | F | 492.0 | 492.1 |
| 1398 | F | 492.0 | 492.1 |
| 1399 | D | 491.0 | 491.1 |
| 1400 | D | 504.0 | 504.2 |
| 1401 | D | 508.0 | 508.1 |
| 1402 | E | 520.0 | 520.1 |
| 1403 | D | 514.0 | 514.0 |
| 1404 | B | 534.0 | 534.0 |
| 1405 | F | 546.9 | 547.1 |
| 1406 | B | 534.0 | 534.0 |
| 1407 | F | 490.0 | 490.1 |
| 1408 | C | 490.0 | 490.1 |
| 1409 | E | 521.0 | 521.2 |
| 1410 | C | 508.0 | 508.6 |
| 1411 | C | 536.0 | 536.6 |
| 1412 | C | 536.0 | 536.6 |
| 1413 | F | 518.0 | 518.2 |
| 1414 | B | 585.1 | 585.2 |
| 1415 | B | 536.0 | 536.6 |
| 1416 | B | 536.0 | 536.6 |
| 1417 | C | 508.0 | 508.6 |
| 1418 | C | 494.9 | 495.1 |
| 1419 | D | 522.1 | 522.2 |
| 1420 | E | 507.0 | 507.2 |
| 1421 | C | 520.0 | 520.6 |
| 1422 | F | 451.9 | 452.3 |
| 1423 | D | 493.0 | 493.4 |
| 1424 | B | 569.0 | 569.6 |
| 1425 | C | 555.0 | 555.5 |
| 1426 | F | 494.0 | 494.3 |
| 1427 | C | 519.0 | 519.7 |
| 1428 | F | 520.0 | 520.1 |
| 1429 | C | 504.0 | 504.1 |
| 1430 | E | 490.0 | 490.1 |
| 1431 | F | 476.0 | 476.1 |
| 1432 | D | 507.0 | 507.1 |
| 1433 | C | 508.0 | 508.2 |
| 1434 | D | 504.0 | 504.2 |
| 1435 | B | 512.0 | 512.2 |
| 1436 | E | 519.0 | 519.2 |
| 1437 | B | 518.0 | 518.2 |
| 1438 | B | 546.0 | 546.1 |
| 2001 | A | 574.1 | 574.2 |
| 2002 | A | 589.1 | 589.3 |
| 2003 | C | 566.7 | 567.3 |
| 2004 | B | 522.0 | 522.1 |
| 2005 | C | 494.5 | 495.1 |
| 2006 | F | 512.6 | 513.1 |
| 2007 | B | 585.1 | 585.3 |
| 2008 | A | 550.7 | 551.3 |
| 2009 | B | 604.6 | 605.3 |
| 2010 | B | 561.6 | 562.3 |
| 2011 | C | 483.6 | 484.3 |
| 2012 | C | 548.6 | 549.2 |
| 2013 | E | 529.0 | 529.1 |
| 2014 | C | 547.0 | 547.1 |
| 2015 | D | 510.0 | 510.1 |
| 2016 | E | 505.0 | 505.2 |
| 2017 | AA | 587.1 | 587.2 |
| 2018 | A | 533.0 | 533.2 |
| 2019 | B | 498.6 | 499.2 |
| 2020 | B | 505.0 | 505.1 |
| 2021 | E | 505.0 | 505.1 |
| 2022 | A | 487.6 | 488.2 |
| 2023 | C | 459.5 | 460.1 |
| 2024 | A | 486.6 | 487.2 |
| 2025 | A | 459.5 | 460.1 |
| 2027 | E | 458.5 | 459.0 |
| 2028 | F | 458.5 | 459.3 |
| 2029 | F | 492.0 | 492.1 |
| 2030 | E | 495.9 | 496.0 |
| 2031 | G0 | 494.0 | 494.0 |
| 2032 | C | 475.5 | 476.0 |
| 2033 | C | 508.0 | 508.0 |
| 2034 | F | 475.5 | 475.9 |
| 2035 | E | 458.5 | 459.4 |
| 2036 | D | 492.0 | 492.6 |
| 2037 | F | 474.5 | 475.1 |
| 2038 | F | 455.5 | 456.7 |
| 2039 | B | 538.0 | 538.2 |
| 2040 | A | 562.0 | 562.5 |
| 2041 | A | 480.0 | 480.3 |
| 2042 | F | 482.5 | 483.6 |
| 2043 | A | 552.0 | 552.2 |
| 2044 | A | 573.0 | 573.2 |
| 2045 | E | 463.6 | 464.6 |
| 2046 | A | 538.0 | 538.3 |
| 2047 | F | 435.9 | 436.4 |
| 2048 | A | 554.0 | 554.3 |
| 2049 | A | 568.0 | 568.3 |
| 2050 | A | 566.0 | 566.1 |
| 2051 | C | 492.0 | 492.1 |
| 2052 | F | 508.0 | 508.0 |
| 2053 | A | 568.0 | 568.1 |
| 2054 | E | 494.0 | 494.6 |
| 2056 | A | 537.0 | 537.0 |
| 2057 | D | 492.0 | 492.0 |
| 2058 | E | 522.0 | 522.0 |
| 3001 | C | 416.9 | 417.2 |
| 3002 | B | 430.9 | 431.1 |
| 3003 | B | 533.0 | 533.32 |
| 3004 | B | 503.0 | 503.3 |
| 3005 | A | 587.1 | 587.3 |
| 3006 | A | 557.1 | 557.3 |
| 3007 | C | 569.1 | 569.3 |
| 3008 | C | 533.0 | 533.2 |
| 3009 | C | 587.1 | 587.2 |
| 3010 | C | 584.1 | 584.2 |
| 3011 | A | 584.1 | 584.3 |
| 3012 | B | 460.9 | 461.2 |
| 3013 | B | 446.9 | 447.1 |
| 3014 | B | 517.0 | 517.2 |
| 3015 | A | 601.1 | 601.2 |
| 3016 | B | 547.0 | 547.2 |
| 3017 | A | 598.1 | 598.2 |
| 3018 | D | 493.9 | 494.1 |
| 3019 | B | 522.0 | 522.1 |
| 3020 | B | 557.0 | 557.3 |
| 3021 | B | 571.0 | 571.3 |
| 3022 | C | 491.0 | 491.1 |
| 3023 | C | 551.0 | 551.2 |
| 4001 | E | 560.1 | 560.1 |
| 4002 | D | 534.1 | 534.2 |
| 4003 | B | 571.1 | 571.2 |
| 4004 | D | 520.0 | 520.2 |

### Determination of Related Protease Inhibition

In vitro inhibition of related proteases was determined using a single-concentration % inhibition screen and/or IC50 determination at relevant enzymes. Assays were based on standard literature methods (see e.g. Shori et al., Biochem. Pharmacol., 1992,43, 1209; Bouckaert et al., European Journal of Medicinal Chemistry 110 (2016) 181). Human serine protease enzyme Plasma Kallikrein, KLK1, FXa, Plasmin, Thrombin and Trypsin were assayed for enzymatic activity using an appropriate fluorogenic substrate at Km concentration, FXIa at fixed substrate concentration of 100 μM and various concentrations of the test compound. Protease activity was measured by monitoring fluorescence emitted from cleaved substrate over 5 min at 25° C. The linear rate of fluorescence increase per minute was expressed as 10 percentage (%) activity. IC₅₀ values were calculated as the concentration of inhibitor giving 50% inhibition of the uninhibited enzyme activity (100%).

Data acquired from this assay are shown in Table 8 using the scale shown in Table 9.

TABLE 8
Enzyme selectivity data

| | IC₅₀ | IC₅₀ | IC₅₀ | IC₅₀ | IC₅₀ | IC₅₀ | IC₅₀ |
|---|---|---|---|---|---|---|---|
| | (human | (human | (human | (human | (human | (human | (human |
| Example | PKal) | KLK1) | plasmin) | FXa) | FXIa) | thrombin) | trypsin) |
| Number | nM | nM | nM | nM | nM | nM | nM |
| 1001 | | | | G3 | | | |
| 1002 | | | | G1 | | | |
| 1003 | | | | G4 | | | |
| 1004 | | | | G3 | | | |
| 1005 | | | | E | | | |
| 1006 | | | | F | | | |
| 1007 | | | | F | | | |
| 1008 | | | | G4 | | | |
| 1009 | | | | G4 | | | |
| 1010 | | | | G4 | | | |
| 1011 | | | | G4 | | | |
| 1012 | | | | F | | | |
| 1013 | | | | C | | | |
| 1014 | | | | F | | | |
| 1015 | | | | F | | | |
| 1019 | | | | G3 | | | |
| 1021 | | | | E | | | |
| 1022 | | | | E | | | |
| 1023 | | | | E | | | |
| 1024 | | | | D | | | |
| 1025 | | | | E | | | |
| 1026 | | | | F | | | |
| 1027 | G4 | G4 | G4 | E | | G4 | |
| 1029 | | | | G4 | | | |
| 1030 | | | | E | | | |
| 1031 | | | | F | | | |
| 1032 | | | | G3 | | | |
| 1035 | G4 | G3 | G4 | F | | G4 | |
| 1036 | | | | G3 | | | |
| 1037 | G4 | G4 | G4 | G4 | | G4 | |
| 1043 | | | | E | | | |
| 1044 | G4 | G3 | G4 | E | | G4 | |
| 1051 | | | | E | | | |
| 1055 | G3 | G3 | G3 | G3 | G3 | | |
| 1074 | | | | F | | | |
| 1076 | | | | F | | | |
| 1077 | | | | E | | | |
| 1078 | | | | E | | | |
| 1079 | | | | F | | | |
| 1085 | | | | G3 | | | |
| 1089 | | | | F | | | |
| 1092 | G3 | G3 | G3 | G3 | G3 | | |
| 1099 | | | | E | | | |
| 1106 | | | | G3 | | | |
| 1109 | | | | G3 | | | |
| 1110 | G3 | F | G3 | F | G3 | | |
| 1111 | | | | F | | | |
| 1112 | G3 | G3 | G3 | G3 | G3 | | |
| 1114 | | | | G3 | | | |
| 1117 | | | | F | | | |
| 1118 | | | | G3 | | | |
| 1121 | | | | G3 | | | |
| 1122 | | | | G3 | | | |
| 1123 | | | | G3 | | | |
| 1125 | G3 | G3 | G3 | F | G3 | | |
| 1126 | | | | F | | | |
| 1127 | | | | F | | | |
| 1128 | | | | F | | | |
| 1129 | G3 | E | G3 | E | G3 | | |
| 1130 | | | | F | | | |
| 1132 | | | | F | | | |
| 1134 | | | | F | | | |
| 1135 | | | | D | | | |
| 1137 | | | | F | | | |
| 1138 | | | | E | | | |
| 1139 | | | | F | | | |
| 1140 | | | | E | | | |
| 1141 | | | | E | | | |
| 1142 | | | | G3 | | | |
| 1143 | | | | E | | | |
| 1144 | | | | D | | | |
| 1145 | | | | F | | | |
| 1146 | | | | F | | | |
| 1147 | | | | F | | | |
| 1148 | | | | F | | | |
| 1149 | | | | G3 | | | |
| 1150 | | | | E | | | |
| 1151 | | | | F | | | |
| 1152 | | | | G3 | | | |
| 1153 | | | | F | | | |
| 1154 | | | | F | | | |
| 1155 | | | | F | | | |
| 1156 | | | | F | | | |
| 1157 | | | | F | | | |
| 1158 | | | | F | | | |
| 1159 | | | | G3 | | | |
| 1160 | | | | G3 | | | |
| 1161 | | | | G3 | | | |
| 1176 | | | | G3 | | | |
| 1177 | | | | G3 | | | |
| 1179 | | G3 | | G3 | | | |
| 1180 | | G3 | | G3 | | | |
| 1181 | | G3 | | G3 | | | |
| 1182 | | G3 | | | | | |
| 1183 | | G3 | | | | | |
| 1184 | | G3 | | | | | |
| 1185 | | | | G3 | | | |
| 1186 | | | | F | | | |
| 1187 | | | | G3 | | | |
| 1188 | | G0 | | G3 | | | |
| 1189 | | D | | G3 | | | |
| 1190 | | G3 | | | | | |
| 1191 | | G0 | | | | | |
| 1192 | | G3 | | | | | |
| 1193 | | G0 | | G3 | | | |
| 1194 | | G0 | | G3 | | | |
| 1195 | | F | | G3 | | | |
| 1196 | | G3 | | | | | |
| 1197 | | G0 | | G3 | | | |
| 1198 | | G0 | | G3 | | | |
| 1199 | | G0 | | F | | | |
| 1200 | | G0 | | F | | | |
| 1201 | | G0 | | F | | | |
| 1202 | | G0 | | G3 | | | |
| 1203 | | G0 | | G3 | | | |
| 1204 | | G0 | | G3 | | | |
| 1205 | | F | | G3 | | | |
| 1206 | | G3 | | G3 | | | |
| 1207 | | G3 | | G3 | | | |
| 1208 | | G3 | | F | | | |
| 1209 | | G0 | | G3 | | | |
| 1210 | | G3 | | G3 | | | |
| 1211 | | G3 | | G3 | | | |
| 1212 | | G3 | | G3 | | | |
| 1213 | | F | | E | | | |
| 1216 | | | | G3 | | | |
| 1217 | | | | F | | | |
| 1218 | | | | G3 | | | |
| 1220 | | | | F | | | |
| 1223 | | | | F | | | |
| 1224 | | | | G3 | | | |
| 1225 | | | | F | | | |
| 1226 | | | | G3 | | | |
| 1227 | | | | F | | | |
| 1229 | | | | F | | | |
| 1230 | | | | F | | | |
| 1231 | | | | G3 | | | |
| 1232 | | | | G3 | | | |
| 1233 | | | | G3 | | | |
| 1234 | | | | E | | | |
| 1235 | G3 | E | G3 | G3 | G3 | | G3 |
| 1236 | | | | G3 | | | |
| 1237 | | | | G3 | | | |
| 1238 | | G3 | | E | | | |
| 1239 | | G3 | | F | | | |
| 1240 | | F | | | | | |
| 1241 | | F | | | | | |
| 1242 | | G3 | | | | | |
| 1243 | | G3 | | | | | |
| 1244 | | F | | | | | |
| 1245 | | F | | G3 | | | |
| 1246 | | G3 | | | | | |
| 1247 | | F | | G3 | | | |
| 1248 | | F | | F | | | |
| 1249 | | G3 | | | | | |
| 1250 | | G3 | | G3 | | | |
| 1251 | | G3 | | | | | |
| 1252 | | G3 | | G3 | | | |
| 1253 | | G3 | | G3 | | | |
| 1254 | | F | | F | | | |
| 1255 | | F | | | | | |
| 1256 | | D | | G3 | | | |
| 1257 | | G3 | | G3 | | | |
| 1258 | | G3 | | G3 | | | |
| 1259 | | F | | | | | |
| 1260 | | G3 | | G3 | | | |
| 1261 | | G3 | | G3 | | | |
| 1262 | | G3 | | F | | | |
| 1263 | | F | | | | | |
| 1264 | | E | | F | | | |
| 1265 | | G3 | | G3 | | | |
| 1266 | | G3 | | G3 | | | |
| 1267 | | F | | G3 | | | |
| 1268 | | F | | | | | |
| 1269 | | F | | | | | |
| 1270 | | F | | | | | |
| 1271 | | G3 | | G3 | | | |
| 1272 | | G3 | | | | | |
| 1273 | | G3 | | | | | |
| 1274 | | G3 | | | | | |
| 1275 | | | | F | | | |
| 1282 | | | | G3 | | | |
| 1285 | | F | | G3 | | | |
| 1286 | | E | | | | | |
| 1287 | | | | F | | | |
| 1299 | | | | F | G3 | | |
| 1304 | | | | G3 | | | |
| 1305 | | | | E | G3 | | |
| 1306 | | | | F | G3 | | |
| 1307 | | | | G3 | | | |
| 1308 | | | | G3 | | | |
| 1309 | | | | G3 | | | |
| 1310 | | | | F | | | |
| 1314 | | | | F | G3 | | |
| 1317 | | G3 | | E | G3 | | |
| 1318 | | F | | E | | | |
| 1319 | | G3 | | D | G3 | | |
| 1320 | | G3 | | G3 | | | |
| 1321 | | G3 | | D | | | |
| 1322 | | G3 | | | | | |
| 1323 | | G3 | | | | | |
| 1324 | | G3 | | | | | |
| 1325 | | G3 | | D | | | |
| 1326 | | G3 | | G3 | | | |
| 1327 | | G3 | | G3 | | | |
| 1328 | | G3 | | E | | | |
| 1329 | | G3 | | G0 | | | |
| 1330 | | G3 | | G3 | | | |
| 1331 | | G3 | | G0 | | | |
| 1332 | | G3 | | G0 | | | |
| 1333 | | G3 | | G0 | | | |
| 1334 | | G3 | | G3 | | | |
| 1335 | | | | G3 | | | |
| 1336 | | | | G3 | | | |
| 1337 | | | | G3 | | | |
| 1338 | | | | G3 | | | |
| 1339 | | | | G3 | | | |
| 1340 | | | | G3 | | | |
| 1341 | | | | F | | | |
| 1342 | | | | G3 | | | |
| 1343 | | | | F | | | |
| 1344 | | | | F | | | |
| 1345 | | | | G3 | | | |
| 1346 | | | | G3 | | | |
| 1347 | | | | E | | | |
| 1350 | | | | E | | | |
| 1351 | | | | G3 | | | |
| 1352 | | | | E | | | |
| 1353 | | | | E | | | |
| 1354 | | | | F | | | |
| 1355 | | | | G3 | | | |
| 1356 | | | | E | | | |
| 1357 | | | | G3 | | | |
| 1358 | | | | E | | | |
| 1359 | | | | G3 | | | |
| 1360 | | | | F | | | |
| 1361 | | | | F | | | |
| 1362 | | | | G3 | | | |
| 1363 | | | | G3 | | | |
| 1364 | | | | G3 | | | |
| 1365 | | | | G3 | | | |
| 1366 | | | | F | | | |
| 1367 | | | | F | | | |
| 1368 | | | | G3 | | | |
| 1369 | | | | G3 | | | |
| 1370 | | | | F | | | |
| 1371 | | | | F | | | |
| 1372 | | | | F | | | |
| 1373 | | | | G3 | | | |
| 1374 | | | | F | | | |
| 1375 | | | | G3 | | | |
| 1376 | | | | E | | | |
| 1377 | | | | E | | | |
| 1378 | | | | F | | F | |
| 1379 | | | | E | | | |
| 1380 | | | | F | | | |
| 1381 | | | | G3 | | | |
| 1382 | | | | E | | | |
| 1383 | | | | E | | | |
| 1384 | | | | G3 | | | |
| 1385 | | | | E | | | |
| 1386 | | | | G3 | | | |
| 1387 | | | | F | | | |
| 1388 | | | | D | | | |
| 1389 | | | | G3 | | | |
| 1390 | | | | G3 | | | |
| 1391 | | | | G3 | | | |
| 1392 | | | | G3 | | | |
| 1393 | | | | D | | | |
| 1394 | | | | C | | | |
| 1395 | | | | G3 | | | |
| 1396 | | | | G3 | | | |
| 1397 | | | | G3 | | | |
| 1398 | | | | G3 | | | |
| 1399 | | | | G3 | | | |
| 1400 | | | | G3 | | | |
| 1401 | | | | G3 | | | |
| 1402 | | | | G3 | | | |
| 1403 | | | | E | | | |
| 1404 | | | | G3 | | | |
| 1406 | | H3 | | H3 | | H2 | H2 |
| 1408 | | H1 | H3 | F | | H2 | H2 |
| 1409 | | H2 | | H3 | | H3 | H3 |
| 1410 | | H3 | H3 | H2 | | H1 | H2 |
| 1411 | | H3 | H3 | H3 | | H3 | H2 |
| 1412 | | H3 | H3 | H2 | | H2 | H2 |
| 1413 | | D | | H3 | | H2 | H3 |
| 1414 | | H1 | | H2 | | H2 | H2 |
| 1415 | | H3 | H3 | H3 | | H3 | H2 |
| 1416 | | H3 | H3 | H2 | | H2 | H1 |
| 1417 | | H3 | H3 | H2 | | H2 | H2 |
| 1418 | | H3 | H3 | H2 | | H3 | H2 |
| 1419 | | H2 | H3 | H2 | | H2 | H2 |
| 1420 | | H2 | H3 | H3 | | H3 | H3 |
| 1421 | | H3 | H3 | H3 | | H3 | H2 |
| 1422 | | H1 | H2 | H2 | | H2 | H1 |
| 1423 | | H1 | H2 | H2 | | H2 | H1 |
| 1424 | | H3 | H3 | H2 | | H3 | H2 |
| 1425 | | H3 | H3 | H3 | | H3 | H2 |
| 1426 | | H1 | H3 | H2 | | H2 | H2 |
| 1427 | | H3 | H3 | H3 | | H3 | H3 |
| 1428 | | H3 | H3 | H3 | | H3 | H3 |
| 1429 | | H1 | H3 | H2 | | H2 | H2 |
| 1430 | | H2 | H3 | H3 | | H3 | H3 |
| 1431 | | H1 | H2 | H1 | | H2 | H2 |
| 1432 | | H1 | H2 | H2 | | H1 | H1 |
| 1433 | | H2 | H3 | H2 | | H3 | H2 |
| 1434 | | H2 | H3 | H2 | | H2 | H2 |
| 1435 | | H3 | H3 | H3 | | H3 | H2 |
| 1436 | | H3 | H3 | H3 | | H3 | H3 |
| 1438 | | H3 | H3 | H3 | | H3 | H2 |
| 2002 | | | | G3 | | | |
| 2004 | | | | G3 | | | |
| 2005 | | G0 | | G3 | | | |
| 2006 | | G3 | | G3 | | | |
| 2007 | | | | G3 | | | |
| 2008 | | | | E | | | |
| 2009 | | | | G3 | | | |
| 2010 | | | | G3 | | | |
| 2011 | | G3 | | | | | |
| 2012 | | G3 | | G3 | | | |
| 2013 | | G3 | | G3 | | | |
| 2017 | G0 | G3 | D | F | F | D | AA |
| 2018 | G3 | G3 | C | G3 | G3 | F | AA |
| 2019 | F | F | B | F | G3 | F | AA |
| 2020 | | | | G3 | G3 | | |
| 2021 | | G3 | | | | | |
| 2022 | | G3 | | | | | |
| 2023 | | G3 | | | | | |
| 2024 | | | | G3 | | | |
| 2025 | | | | F | | | |
| 2027 | | | | G3 | | | |
| 2028 | | | | G3 | | | |
| 2029 | | | | G3 | | | |
| 2030 | | | | F | | | |
| 2032 | | | | G3 | | | |
| 2033 | | | | G3 | | | |
| 2035 | | | | F | | | |
| 2036 | | | | G3 | | | |
| 2037 | | H3 | | H3 | | H3 | H3 |
| 2038 | | H3 | | H3 | | H3 | H3 |
| 2039 | | | | G3 | | | |
| 2040 | | | | F | | | |
| 2041 | | H3 | | H2 | | H2 | H1 |
| 2042 | | H2 | H3 | H3 | | H2 | H3 |
| 2043 | | H2 | | H2 | | H2 | H1 |
| 2044 | | D | | H3 | | H2 | H1 |
| 2045 | | H2 | H3 | H2 | | H2 | H2 |
| 2046 | | H2 | H3 | H3 | | H3 | H3 |
| 2047 | | H3 | H3 | H3 | | H3 | H2 |
| 2048 | | H3 | H2 | H2 | | H3 | H1 |
| 2049 | | H2 | H2 | H1 | | H3 | H1 |
| 2050 | | H3 | H3 | H2 | | H2 | H1 |
| 2051 | | H2 | H3 | H2 | | H3 | H1 |
| 2052 | | H3 | H3 | H3 | | H3 | H3 |
| 2053 | | H2 | H2 | H2 | | H3 | H1 |
| 2054 | | H1 | H3 | H3 | | H3 | H2 |
| 2056 | | H3 | H1 | H2 | | H3 | H1 |
| 2057 | | E | H3 | H2 | | H2 | H3 |
| 2058 | | | | G3 | | G3 | |
| 3003 | | G0 | | G3 | | | |
| 3004 | | G0 | | E | | | |
| 3005 | | G0 | | F | | | |
| 3006 | | G0 | | D | | | |
| 3007 | | | | E | | | |
| 3008 | | | | E | | | |
| 3009 | | | | E | | | |
| 3010 | | | | E | | | |
| 3011 | | | | F | | | |
| 3012 | | | | D | | | |
| 3013 | | | | C | | | |
| 3014 | | | | G3 | | | |
| 3015 | | | | G3 | G3 | | |
| 3016 | | | | G3 | G3 | | |
| 3017 | | | | F | | | |
| 3018 | | G3 | | F | | | |
| 3019 | | G3 | | E | | | |
| 3020 | | | | F | | | |
| 3021 | | | | F | | | |
| 3022 | | | | F | | | |
| 4001 | | | | G3 | | | |
| 4002 | | G3 | | G3 | | | |

TABLE 9
Scale used to present enzyme selectivity data

| Category | IC50 (nM) |
|---|---|
| AA | 1-10 |
| A | 10-100 |
| B | 100-300 |
| C | 300-1,000 |
| D | 1,000-3,000 |
| E | 3,000-10,000 |
| F | 10,000-40,000 |
| G0 | >4,000 |
| G1 | >10,000 |
| G2 | >20,000 |
| G3 | >40,000 |
| G4 | >100,000 |
| H1 | <20% activity at 40 μM |
| H2 | 20%-80% activity at 40 μM |
| H3 | >80% activity at 40 μM |

### Pharmacokinetics

Pharmacokinetic studies of the compounds in Table 10 were performed to assess the pharmacokinetics following a single intravenous dose and a single oral dose in male Sprague-Dawley rats. Two rats were given a single intravenous dose of 1 ml/kg of a nominal 1 mg/ml (1 mg/kg) composition of test compound in 10% DMSO/10% Cremophor EL/80% SWFI vehicle. Two rats were given a single oral dose of 5 mL/kg of a nominal 1 mg/ml (5 mg/kg) composition of test compound in 10% DMSO/10% Cremophor EL/80% SWFI vehicle.

Following intravenous dosing, blood samples were collected over a period of 12 h. Sample times were 2, 5, 15 and 30 min then 1, 2, 4, 6, and 12 h. Following oral dosing, blood samples were collected over a period of 24 h. Sample times were 5, 15 and 30 min then 1, 2, 4, 6, 8, 12 and 24 h.

Following collection, blood samples were centrifuged and the plasma fraction analysed for concentration of test compound by LCMS. Oral bioavailability and half-life calculations from these studies were obtained using Phoenix WinNonlin (v8.0) and are shown below:

TABLE 10
Pharmacokinetic Data

| Example | Dose iv | Dose po | | T½ |
|---|---|---|---|---|
| Number | (mg/kg) | (mg/kg) | F % | (min) |
| 1024 | 1.1 | 5.3 | 36% | 328 |
| 1055 | 1.1 | 5.6 | 55% | 277 |
| 1128 | 0.9 | 4.5 | 27% | 273 |
| 1149 | 0.9 | 4.6 | 56% | 133 |
| 1220 | 1.0 | 2.9 | 22% | 70 |
| 1222 | 1.0 | 5.3 | 5% | 35 |
| 2039 | 1.2 | 6.0 | 27% | 45 |
| 2048 | 1.0 | 5.0 | 57% | 49 |
| 2049 | 1.0 | 4.6 | 28% | 85 |

1-26. (canceled)

27. A compound of Formula (I), 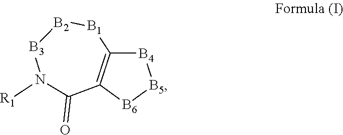
wherein:
B₁ is a carbon centre or nitrogen centre; and either:
(i) B₂ and B₃ together form a single bond between B₁ and the nitrogen atom to which R₁ is attached, or
(ii) B₂ is a carbon centre or a nitrogen centre, and B₃ is a single bond between B₂ and the nitrogen atom to which Ri is attached,
wherein, when B₂ is a carbon centre or nitrogen centre, B₁ is connected to B₂ via either a single or double bond;
wherein B₁, where possible, is optionally substituted with 1 or 2 substituents that are alkyl, cycloalkyl, alkoxy, OH, CF₃, CN, or halo, or 1 oxo substituent, or 2 substituents which, together with B₁ to which they are both attached, form a 3-, 4-, or 5-membered non-aromatic monocyclic carbon-containing ring, optionally wherein one or two ring members are N, S, or O;
wherein, when B₂ is a carbon centre, B₂ is optionally substituted with 1 or 2 substituents that are alkyl, cycloalkyl, alkoxy, OH, CF₃, CN, or halo, or 1 oxo substituent;
and:
(i) B₄ is N(R₂), B₅ is N, and B₆ is C(R₃), and B₄ is connected to B₅ via a single bond and B₅ is connected to B₆ via a double bond;
(ii) B₄ is C(R₂), B₅ is N, and B₆ is N(R₃), and B₄ is connected to B₅ via a double bond and B₅ is connected to B₆ via a single bond; or
(iii) B₄ is C(R₂), B₅ is C(H), and B₆ is N(R₃), and B₄ is connected to B₅ via a double bond and B₅ is connected to B₆ via a single bond;
R₂ is H, —(CH₂)₀₋₃—C(═O)—N(R₄)(R₅), —C(═O)—O—R₆, —(CH₂)₀₋₃—NR₁₄—C(═O)R₁₅, —(CH₂)₀₋₃—NR₃₄—S(═O)₂R₃₅, alkyl, cycloalkyl, heterocycloalkyl, alkoxy, —OH, —CF₃, halo, aryl, heteroaryl, —CH₂—O—R₁₆ or —CN;
wherein R₄ is H, alkyl, cycloalkyl, and heterocycloalkyl, and
R₅ is alkyl, —OH, —S(═O)₂-(small alkyl), —(CH₂)₁₋₃—(C═O)—OR₃₃, —(CH₂)₁₋₃—(C═O)—N(R₃₆)(R₃₇), —(CH₂)₀₋₃-cycloalkyl^{b}, —(CH₂)₀₋₃-heterocycloalkyl, (CH(alkyl))-heterocycloalkyl, —(CH₂)₀₋₃-polycycloalkyl, —(CH₂)₀₋₃-aryl, —(C(H)(phenyl))-alkyl, (CH₂)₀₋₃heteroaryl^{a}, —(C(H)(heteroaryl))-alkyl, —(C(H)(alkyl))-(C—O)—OR₃₈, —(C(H)(alkyl))-(C═O)—N(R₃₉)(R₄₀), —(C(H)(heteroaryl))-(C═O)—OR₂₈, or (C(H)(heteroaryl))-(C═O)—N(R₄₁)(R₄₂);
or wherein R₄ and R₅ together with the nitrogen atom to which they are attached form a carbon-containing 4-, 5-, 6-, or 7-membered heterocyclic ring which is saturated or unsaturated with 1 or 2 double bonds, optionally containing one or two additional heteroatoms that are N, O, or S, wherein the ring is optionally substituted with 1, 2, or 3 substituents that are oxo, alkyl, alkoxy, OH, halo or CF_{3;}
R₂₈, R₃₃ R₃₆, R₃₇, R₃₈, R₃₉, R₄₀, R₄₁, and R₄₂ are each independently H or small alkyl;
R₆ is H, alkyl, —(CH₂)₀₋₃-cycloalkyl, —(CH₂)₀₋₃-heterocycloalkyl, (CH(alkyl))-heterocycloalkyl, —(CH₂)₀₋₃-polycycloalkyl, —(CH₂)₀₋₃-aryl, —(C(H)(phenyl))-alkyl, (CH₂)₀₋₃heteroaryl, or —(C(H)(heteroaryl))-alkyl;
R₁₄ is H, alkyl, cycloalkyl, or heterocycloalkyl;
R₁₅ is H, alkyl, —(CH₂)₀₋₃-cycloalkyl, —(CH₂)₀₋₃-heterocycloalkyl, (CH(alkyl))-heterocycloalkyl, —(CH₂)₀₋₃-aryl, —(C(H)(phenyl))-alkyl, —(CH₂)₀₋₃heteroaryl^{a}, or —(C(H)(heteroaryl))-alkyl;
R₁₆ is H, alkyl, cycloalkyl, —(CH₂)₁₋₃—(C═O)—OH, heterocycloalkyl, or —(CH₂)₀₋₃heteroaryl;
R₃₄ is H, alkyl, cycloalkyl, or heterocycloalkyl;
R₃₅ is H, alkyl, —(CH₂)₀₋₃-cycloalkyl, —(CH₂)₀₋₃-heterocycloalkyl, (CH(alkyl))-heterocycloalkyl, —(CH₂)₀₋₃-aryl, —(C(H)(phenyl))-alkyl, —(CH₂)₀₋₃heteroaryl^{a}, or —(C(H)(heteroaryl))-alkyl;
R₁ is:
(i) Formula (II), wherein Formula (II) is represented by Formula (IIb), 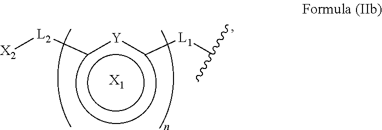
wherein:
n is 1;
L₁ is a single bond;
Y is C, N, O or S;
X₁ is phenyl or a 5- or 6-membered carbon-containing aromatic ring containing one, two or three ring members that are N, S, or O;
wherein X₁ is optionally substituted with 1 or 2 substituents that are independently alkyl, cycloalkyl, alkoxy, OH, O(cyclopropyl), CF₃, or halo;
L₂ is a linker that is a single bond, —(CH₂)₁₋₃—, —(CH)(alkyl)-, —C(alkyl)(alkyl)-, —C(═O)—, or —SO₂—; and
X₂ is:
(a) —NR₂₀R_{21,}
wherein R₂₀ is H, alkyl, or —(CH₂)₀₋₃—R_{22;}
wherein R₂₁ is H, alkyl, or —(CH₂)₀₋₃—R₂₃;
wherein R₂₂ is cycloalkyl, heterocycloalkyl, aryl, heteroaryl, 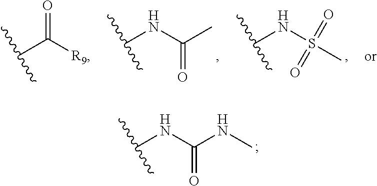
wherein R₂₃ is cycloalkyl, heterocycloalkyl, aryl, heteroaryl, 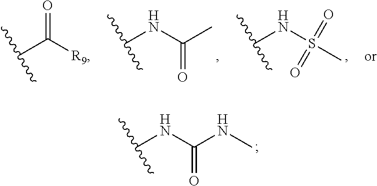
or
(b) 2-pyridone, 4-pyridone, or a non-aromatic, carbon-containing mono- or bi-cyclic ring containing 5, 6, or 7 ring members, wherein at least one ring member is independently N, S, or O, which is saturated or unsaturated with 1 or 2 double bonds which is optionally substituted with 1 or 2 substituents R₁₈ and R₁₉, wherein R₁₈ and R₁₉ are independently alkyl, cycloalkyl, alkoxy, OH, oxo, CF₃, or halo,
or wherein R₁₈ and R₁₉, where possible, together with a carbon atom to which they are both attached form a 3-, 4-, or 5-membered non-aromatic monocyclic carbon-containing ring, optionally wherein one or two ring members are N, S, or O, and X₂ is further optionally substituted with 1 or 2 substituents R₂₉ and R₃₀, wherein R₂₉ and R₃₀ are independently alkyl, cycloalkyl, alkoxy, OH, oxo, CF₃, or halo; or,
(ii) Formula (III) comprising an aromatic ring A fused to a non-aromatic ring A′, 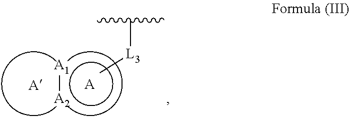
wherein:
L₃ is a single bond;
A₁ and A₂ are both carbon;
A is phenyl or a 5- or 6-membered carbon-containing aromatic ring containing one, two or three ring members that are N, S, or O;
wherein A is optionally substituted with 1 or 2 substituents that are independently alkyl, cycloalkyl, alkoxy, OH, O(cyclopropyl), CF₃, or halo;
A′ is a non-aromatic carbon-containing monocyclic ring containing 5, 6, or 7, ring members, wherein at least one ring member is independently N, S, or O;
wherein A′ is optionally substituted with one R₇, wherein R₇ is alkyl, alkoxy, or —(CH₂)₀₋₃—R₈,
wherein R₈ is cycloalkyl, heterocycloalkyl, aryl, heteroaryl, 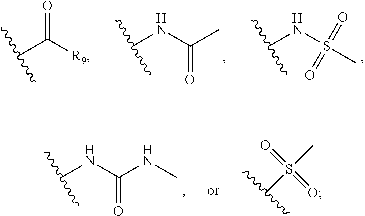
wherein R₉ is H, alkyl, OH, N (R₃₁)(R₃₂), or alkoxy;
wherein R₃₁ is H or small alkyl;
wherein R₃₂ is H, small alkyl, or —(CH₂)₀₋₃heteroaryl;
wherein A′ is optionally substituted with 1, 2, or 3 further substituents R₁₀, R₁₁, and R₁₇, wherein R₁₀, R₁₁, and R₁₇, are independently alkyl, cycloalkyl, alkoxy, OH, oxo, CF₃, or halo,
or wherein A′ is optionally substituted with 1 or 2 further substituents R₂₄ and R₂₇ that are independently alkyl, cycloalkyl, alkoxy, OH, oxo, CF₃, or halo and 2 further substituents R₂₅ and R₂₆, wherein R₂₅ and R₂₆ together with a carbon atom to which they are both attached form a 3-, 4-, or 5-membered non-aromatic monocyclic carbon-containing ring, optionally wherein one or two ring members are N, S, or O,
or wherein A′ is optionally substituted with 1 or 2 further substituents R₄₃ and R₄₄ that are independently alkyl, cycloalkyl, alkoxy, OH, oxo, CF₃, or halo, and two different carbon ring members within A′ are connected by a bridging —CH₂-group; and
R₃ is phenyl, or a 5- or 6-membered carbon-containing aromatic ring containing one, two or three ring members that are N, S, or O, wherein R₃ is substituted with one, two, or three substituents that are independently small alkyl^{b}, small alkoxy, cyclopropyl, OH, NH₂, CF₃, halo, —C(—O)—NH₂, —C(═O)—N(H)(small alkyl), —C(═O)—N(small alkyl)(small alkyl), or CN;
wherein R₃ is not 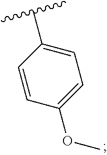 [SMILES: *c1ccc(OC)cc1]
and
alkyl is a linear saturated hydrocarbon having up to 10 carbon atoms (C₁-C₁₀) or a branched saturated hydrocarbon of between 3 and 10 carbon atoms (C₃-C₁₀); alkyl is optionally substituted with 1, 2 or 3 substituents that are independently cyclopropyl, (C₁-C₆) alkoxy, OH, —NR₁₂R₁₃, —C(═O) NR₁₂R₁₃, CN, CF₃, or halo;
small alkyl is a linear saturated hydrocarbon having up to 4 carbon atoms (C₁-C₄) or a branched saturated hydrocarbon of between 3 and 4 carbon atoms (C₃-C₄); small alkyl is optionally substituted with 1 or 2 substituents that are independently cyclopropyl, OH, CN, CF₃, NH₂, —C(═O)NH₂, or halo;
small alkyl^{b} is a linear saturated hydrocarbon having up to 4 carbon atoms (C₁-C₄) or a branched saturated hydrocarbon of between 3 and 4 carbon atoms (C₃-C₄); small alkyl is optionally substituted with 1 or 2 substituents that are independently cyclopropyl, OH, OMe, OEt, OⁱPr, OⁿPr, O(cyclopropyl), OCF₃, OCHF₂, CN, CF₃, NH₂, —C(—O)NH₂, or halo;
alkoxy is OCF₃, O(cyclopropyl), a linear O-linked hydrocarbon of between 1 and 6 carbon atoms (C₁-C₆), or a branched O-linked hydrocarbon of between 3 and 6 carbon atoms (C₃-C₆); alkoxy is optionally substituted with 1 or 2 substituents that are independently cyclopropyl, OH, CN, CF₃, or halo;
small alkoxy is OCF₃, O(cyclopropyl), a linear O-linked hydrocarbon of between 1 and 4 carbon atoms (C₁-C₄), or a branched O-linked hydrocarbon of between 3 and 4 carbon atoms (C₃-C₄); small alkoxy is optionally substituted with 1 or 2 substituents that are independently cyclopropyl, OH, CN, CF₃, or halo;
aryl is phenyl, biphenyl or naphthyl; aryl is optionally substituted with 1, 2 or 3 substituents that are independently alkyl, alkoxy, methylenedioxy, ethylenedioxy, OH, halo, CN, (CH₂)₀₋₃—NR₁₂R₁₃, OCF₃ or CF_{3;}
polycycloalkyl is a bicyclic saturated hydrocarbon ring system of between 5 and 8 carbon atoms (C₅-C₈), wherein the bicyclic ring is bridged, or is adamantyl; polycycloalkyl is optionally substituted with 1 or 2 substituents that are independently alkyl, (C₁-C₆) alkoxy, OH, CN, CF₃, or halo;
cycloalkyl is a monocyclic saturated hydrocarbon ring of between 3 and 6 carbon atoms (C₃-C₆); cycloalkyl is optionally substituted with 1 or 2 substituents that are independently alkyl, (C₁-C₆) alkoxy, OH, CN, CF₃, or halo
cycloalkyl^{a} is a monocyclic saturated hydrocarbon ring of between 3 and 6 carbon atoms (C₃-C₆); cycloalkyl is optionally substituted with 1 or 2 substituents that are independently alkyl, (C₁-C₆) alkoxy, OH, CN, CF₃, halo, or —(C—O)—OH;
cycloalkyl^{b} is a monocyclic saturated hydrocarbon ring of between 3 and 6 carbon atoms (C₃-C₆); cycloalkyl is optionally substituted with 1 or 2 substituents that are independently alkyl, (C₁-C₆) alkoxy, OH, NH₂, NH(small alkyl), CN, CF₃, halo, or —(C═O)—OH;
halo is F, Cl, Br, or I;
heteroaryl is a 5- or 6-membered carbon-containing aromatic ring containing one, two or three ring members that are N, S, or O; wherein heteroaryl is optionally substituted with 1, 2 or 3 substituents that are independently alkyl, alkoxy, cyclopropyl, OH, halo, CN, or CF_{3;}
heteroaryl^{a} is a 5-, 6-, 9- or 10-membered mono- or bi-cyclic aromatic ring containing, where possible, 1, 2, 3 or 4 ring members that are independently N, S, or O; wherein heteroaryl^{a} is optionally substituted with 1, 2 or 3 substituents that are independently alkyl, alkoxy, cyclopropyl, OH, halo, CN, or CF_{3;}
heterocycloalkyl is a non-aromatic carbon-containing mono- or bi-cyclic ring containing 3, 4, 5, 6, or 7 ring members, wherein at least one ring member is independently N, S, or O; heterocycloalkyl is optionally substituted with 1 or 2 substituents that are independently alkyl, cyclopropyl, (C₁-C₆) alkoxy, OH, CN, CF₃, oxo, or halo; wherein when heterocycloalkyl is a bi-cyclic ring system, the bicyclic ring system is fused, bridged or spiro;
each R₁₂ and R₁₃ is independently:
(i) H,
(ii) a linear saturated hydrocarbon having up to 10 carbon atoms (C₁-C₁₀) or a branched saturated hydrocarbon of between 3 and 10 carbon atoms (C₃-C₁₀); which is optionally substituted with 1, 2 or 3 substituents that are independently cyclopropyl, (C₁-C₆) alkoxy, OH, CN, CF₃, or halo,
(iii) a monocyclic saturated hydrocarbon ring of between 3 and 6 carbon atoms (C₃-C₆); which is optionally substituted with 1 or 2 substituents that are independently small alkyl, (C₁-C₆) alkoxy, OH, CN, CF₃, or halo, or
(iv) a non-aromatic carbon-containing mono- or bi-cyclic ring containing 3, 4, 5, 6, or 7 ring members, wherein at least one ring member is independently N, S, or O; which is optionally substituted with 1 or 2 substituents that are independently small alkyl, cyclopropyl, (C₁-C₆) alkoxy, OH, CN, CF₃, oxo, or halo; wherein when the non-aromatic carbon-containing ring is a bi-cyclic ring system, the bicyclic ring system is fused, bridged or spiro;
or a tautomer, isomer, stereoisomer, deuterated analogue, or pharmaceutically acceptable salt and/or solvate thereof.

28. The compound according to claim 27, or a tautomer, isomer, stereoisomer, a deuterated analogue, or a pharmaceutically acceptable salt and/or solvate thereof, wherein:
(i) B₁ is a carbon centre, and B₂ and B₃ together form a single bond between B₁ and the nitrogen atom to which Ri is attached,
(ii) B₁ is a carbon centre and B₂ is a carbon centre, and B₁ is connected to B₂ via a single bond, or
(iii) B₁ is a nitrogen centre and B₂ is a carbon centre and B₁ is connected to B₂ via a double bond.

29. The compound according to claim 28, or a tautomer, isomer, stereoisomer, a deuterated analogue, or a pharmaceutically acceptable salt and/or solvate thereof, wherein B₁ is a carbon centre and B₂ is a carbon centre, and B₁ is connected to B₂ via a single bond, or wherein B₁ is a nitrogen centre and B₂ is a carbon centre and B₁ is connected to B₂ via a double bond.

30. The compound according to claim 27, or a tautomer, isomer, stereoisomer, a deuterated analogue, or a pharmaceutically acceptable salt and/or solvate thereof, wherein:
(i) B₄ is N(R₂), B₅ is N, and B₆ is C(R₃), and B₄ is connected to B₅ via a single bond and B₅ is connected to B₆ via a double bond; or
(ii) B₄ is C(R₂), B₅ is N, and B₆ is N(R₃), and wherein B₄ is connected to B₅ via a double bond and B₅ is connected to B₆ via a single bond.

31. The compound according to claim 27, or a tautomer, isomer, stereoisomer, a deuterated analogue, or a pharmaceutically acceptable salt and/or solvate thereof, wherein R₂ is —C(═O)—N(R₄)(R₅), —C(═O)—O—R₆, —CH₂—NH—C(═O)CH₃, —CH₂—NH—S(═O)₂R₃₅, —CH₂—O—R₁₆, —CH₂—OH, —CN, or

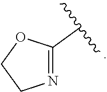 [SMILES: *C1=NCCO1]

32. The compound according to claim 27, or a tautomer, isomer, stereoisomer, a deuterated analogue, or a pharmaceutically acceptable salt and/or solvate thereof, wherein R₂ is —C(═O)—N(R₄)(R₅), —CH₂—NH—C(═O)CH₃, —CH₂—NH—S(═O)₂R₃₅, —CH₂—O—R₁₆, —CH₂—OH, or

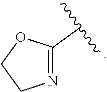 [SMILES: *C1=NCCO1]

33. The compound according to claim 27, or a tautomer, isomer, stereoisomer, a deuterated analogue, or a pharmaceutically acceptable salt and/or solvate thereof, wherein R₂ is —C(═O)—N(R₄)(R₅).

34. The compound according to claim 27, or a tautomer, isomer, stereoisomer, a deuterated analogue, or a pharmaceutically acceptable salt and/or solvate thereof, wherein:
R₄ is H or small alkyl, and
R₅ is alkyl, —OH, —S(═O)₂-(small alkyl), —(CH₂)₁₋₃—(C═O)—OH, (CH₂)₀₋₃-cycloalkyl^{b}, —(CH₂)₀₋₃-heterocycloalkyl, —(CH(alkyl))-heterocycloalkyl, (CH₂)₀₋₃-polycycloalkyl, —(CH₂)₀₋₃-aryl, —(C(H)(phenyl))-alkyl, (CH₂)₀₋₃heteroaryl^{a}, —(C(H)(heteroaryl))-alkyl, —(C(H)(alkyl))-(C═O)—OH, or —(C(H)(heteroaryl))-(C═O)—OR_{28;}
or wherein R₄ and R₅ together with the nitrogen atom to which they are attached form a carbon-containing 4-, 5-, or 6-membered saturated heterocyclic ring, optionally containing an additional heteroatom that is N or O, which is optionally mono- or di-substituted with substituents that are oxo, alkyl, alkoxy, OH, halo or CF_{3;}
R₆, where present, is H, alkyl, —(CH₂)₀₋₃-cycloalkyl, (CH₂)₀₋₃-heterocycloalkyl, —(CH(alkyl))-heterocycloalkyl, —(CH₂)₀₋₃-polycycloalkyl, —(CH₂)₀₋₃-aryl, —(C(H)(phenyl))-alkyl, —(CH₂)₀₋₃heteroaryl, or (C(H)(heteroaryl))-alkyl;
R₁₆ is H, small alkyl, —(CH₂)—(C═O)—OH, heterocycloalkyl, or —(CH₂)-heteroaryl; and
R₃₅ is H, small alkyl, or cyclopropyl.

35. The compound according to claim 27, or a tautomer, isomer, stereoisomer, a deuterated analogue, or a pharmaceutically acceptable salt and/or solvate thereof, wherein R₅ is alkyl, —OH, —S(═O)₂-(small alkyl), —(CH₂)₁₋₃—(C═O)—OR₃₃, —(CH₂)₁₋₃—(C—O)—N(R₃₆)(R₃₇), —(CH₂)₀₋₃-cycloalkyl^{b}, —(CH₂)₀₋₃-heterocycloalkyl, —(CH(alkyl))-heterocycloalkyl, —(CH₂)₀₋₃-polycycloalkyl, —(C(H)(alkyl))-(C═O)—OR₃₈, or —(C(H)(alkyl))-(C═O)—N(R₃₉)(R₄₀).

36. The compound according to claim 27, or a tautomer, isomer, stereoisomer, a deuterated analogue, or a pharmaceutically acceptable salt and/or solvate thereof, wherein X₁ is phenyl or pyridyl.

37. The compound according to claim 27, or a tautomer, isomer, stereoisomer, a deuterated analogue, or a pharmaceutically acceptable salt and/or solvate thereof, wherein X₂ is 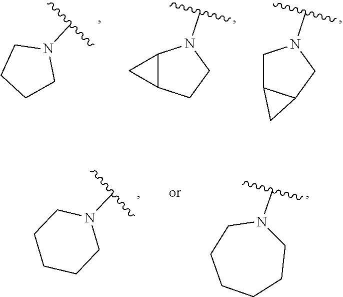
wherein X₂ is optionally substituted with 1 or 2 substituents R₁₈ and R₁₉, wherein R₁₈ and R₁₉ are independently alkyl, cycloalkyl, alkoxy, OH, oxo, CF₃, or halo,
or wherein R₁₈ and R₁₉, where possible, together with a carbon atom to which they are both attached form a 3-, 4-, or 5-membered non-aromatic monocyclic carbon-containing ring, optionally wherein one or two ring members are N, S, or O, and X₂ is further optionally substituted with 1 or 2 substituents R₂₉ and R₃₀, wherein R₂₉ and R₃₀ are independently alkyl, cycloalkyl, alkoxy, OH, oxo, CF₃, or halo.

38. The compound according to claim 27, or a tautomer, isomer, stereoisomer, a deuterated analogue, or a pharmaceutically acceptable salt and/or solvate thereof, wherein Formula (II) is represented by Formula (IIa), 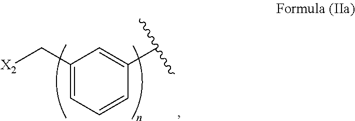
wherein:
n is 1;
X₂ is a non-aromatic, carbon-containing mono- or bi-cyclic ring containing 5, 6, or 7 ring members, wherein at least one ring member is independently N, S, or 0;
wherein X₂ is optionally substituted with 1 or 2 substituents R₁₈ and R₁₉, wherein R₁₈ and R₁₉ are independently alkyl, cycloalkyl, alkoxy, OH, oxo, CF₃, or halo,
or wherein R₁₈ and R₁₉, where possible, together with a carbon atom to which they are both attached form a 3-, 4-, or 5-membered non-aromatic monocyclic carbon-containing ring, optionally wherein one or two ring members are N, S, or O.

39. The compound according to claim 27, or a tautomer, isomer, stereoisomer, a deuterated analogue, or a pharmaceutically acceptable salt and/or solvate thereof, wherein A is phenyl or a 6-membered carbon-containing aromatic ring containing one, two or three nitrogen ring members.

40. The compound according to claim 27, or a tautomer, isomer, stereoisomer, a deuterated analogue, or a pharmaceutically acceptable salt and/or solvate thereof, wherein A′ is a non-aromatic carbon-containing monocyclic ring containing 5, 6, or 7 ring members, wherein at least one ring member is N.

41. The compound according to claim 27, or a tautomer, isomer, stereoisomer, a deuterated analogue, or a pharmaceutically acceptable salt and/or solvate thereof, wherein R₁ is Formula (III).

42. The compound according to claim 27, or a tautomer, isomer, stereoisomer, a deuterated analogue, or a pharmaceutically acceptable salt and/or solvate thereof, wherein R₇ is (C₁-C₃)alkyl and is bonded to A′ via a N ring member.

43. The compound according to claim 27, or a tautomer, isomer, stereoisomer, a deuterated analogue, or a pharmaceutically acceptable salt and/or solvate thereof, wherein, when R₃ is a substituted six-membered ring, R₃ is not substituted with an methoxy group at the para-position to the attachment to B_{6.}

44. The compound according to claim 27, or a tautomer, isomer, stereoisomer, a deuterated analogue, or a pharmaceutically acceptable salt and/or solvate thereof, wherein R₃ is substituted phenyl, substituted pyridyl, or substituted thiophene.

45. The compound according to claim 27, or a tautomer, isomer, stereoisomer, a deuterated analogue, or a pharmaceutically acceptable salt and/or solvate thereof, wherein R₃ is substituted with one or two substituents that are independently small alkyl^{b}, small alkoxy, CF₃, halo, —C(═O)—NH₂, or CN.

46. The compound according to claim 27, or a tautomer, isomer, stereoisomer, a deuterated analogue, or a pharmaceutically acceptable salt and/or solvate thereof, wherein R₃ is:

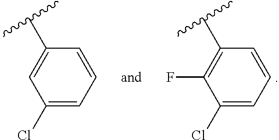 [SMILES: *c1cccc(Cl)c1 ; *c1cccc(Cl)c1F]

47. A compound selected from Table 1a, Table 1b, Table 1c, Table 1d, Table 2a, Table 2b, Table 2c, Table 2d, Table 3a, Table 3b, Table 3c, Table 3d, or Table 4, or a pharmaceutically acceptable salt, solvate, or solvate of a salt thereof.

48. A pharmaceutical composition comprising: a compound, or a pharmaceutically acceptable salt and/or solvate thereof, according to claim 27, and at least one pharmaceutically acceptable excipient.

49. A method for treating a disease or condition in which Factor XIIa activity is implicated; wherein the disease or condition in which Factor XIIa activity is implicated is a bradykinin-mediated angioedema; wherein the bradykinin-mediated angioedema is hereditary angioedema, comprising administering a compound, or a pharmaceutically acceptable salt and/or solvate thereof, according to claim 27 to a patient in need thereof.

50. A method for treating a disease or condition in which Factor XIIa activity is implicated; wherein the disease or condition in which Factor XIIa activity is implicated is a bradykinin-mediated angioedema; wherein the bradykinin-mediated angioedema is non hereditary, comprising administering a compound, or a pharmaceutically acceptable salt and/or solvate thereof, according to claim 27 to a patient in need thereof.

51. A method for treating a disease or condition in which Factor XIIa activity is implicated; wherein the disease or condition in which Factor XIIa activity is implicated is a thrombotic disorder, comprising administering a compound, or a pharmaceutically acceptable salt and/or solvate thereof, according to claim 27 to a patient in need thereof.

52. The compound according to claim 27, wherein the stereoisomer is an enantiomer, diastereoisomer, or a racemic or scalemic mixture thereof.
